(19) United States
(12) Patent Application Publication
(10) Pub. No.: US 2025/0114374 A1
(43) Pub. Date: Apr. 10, 2025

(54) BCL-2 INHIBITORS

(71) Applicant: Newave Pharmaceutical Inc., Pleasanton, CA (US)

(72) Inventor: Yi Chen, Pleasanton, CA (US)

(21) Appl. No.: 18/721,999

(22) PCT Filed: Dec. 19, 2022
(86) PCT No.: PCT/US2022/053328

§ 371 Date: Jun. 20, 2024

Related U.S. Application Data
(60) Provisional application No. 63/298,726, filed on Jan. 12, 2022.

(30) Foreign Application Priority Data
Dec. 20, 2021 (WO) PCT/US2021/064278
Jun. 22, 2022 (WO) PCT/US2022/034518

(51) Int. Cl. A61K 31/553; A61P 35/02; C07D 487/04; C07D 498/02

(52) U.S. Cl. CPC A61K 31/553; A61P 35/02; C07D 487/04; C07D 498/02

(57) ABSTRACT

The disclosure includes compounds of Formulae (1) wherein Q₁, Q₂, Q₃, Q₄, Q₅, Q₆, Q₇, Q₈, Qₓ, R₁, R₂, R₃, R₄, R₅, R₆, R₇, R₉, R₁₀, R₁₁, Rₓ, a, b, c, g, j, k, m, n, u, v, L, Z₁, Z₂, Z₄, and Z₅ are defined herein. Also disclosed is a method for treating a neoplastic disease and autoimmune disease with these compounds.

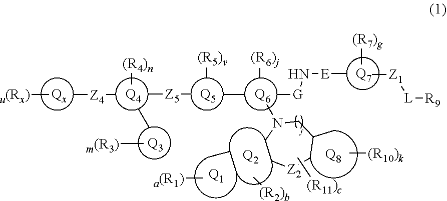

### REFERENCE TO RELATED APPLICATIONS

This application claims priority to, and the benefit of the filing date of International Patent Application No. PCT/US2021/064278, filed on Dec. 20, 2021, U.S. Provisional Patent Application No. 63/298,726, filed on Jan. 12, 2022, International Patent Application No. PCT/US2022/034518, filed on Jun. 22, 2022, and U.S. Provisional Patent Application No. 63/413,569, filed on Oct. 5, 2022, the entire contents of each of the above-referenced applications are incorporated herein by reference.

### BACKGROUND OF THE INVENTION

Apoptosis, or programmed cell death, is a conserved and regulated process that is the primary mechanism for the removal of aged, damaged and unnecessary cells. The ability to block apoptotic signaling is a key hallmark of cancer and is thus important for oncogenesis, tumor maintenance and chemoresistance [Hanahan, D. & Weinberg, R. A. The hallmarks of cancer. Cell 100, 57-70 (2000).]. Dynamic binding interactions between prodeath (for example, BCL-2-associated X protein (BAX), BCL-2 antagonist/killer 1 (BAK), BCL-2-associated agonist of cell death (BAD), BCL-2-like 11 (BIM), NOXA and BCL-2 binding component 3 (PUMA)) and prosurvival (BCL-2, BCL-XL, BCL-2-like 2 (BCL-W), myeloid cell leukemia sequence 1 (MCL-1) and BCL-2-related protein A1 (BFL-1)) proteins in the BCL-2 family control commitment to programmed cell death. Altering the balance among these opposing factions provides one means by which cancer cells undermine normal apoptosis and gain a survival advantage [Youle, R. J. & Strasser, A. The BCL-2 protein family: opposing activities that mediate cell death. Nat. Rev. Mol. Cell Biol. 9, 47-59 (2008)].

BCL-2, the first identified apoptotic regulator, was originally cloned from the breakpoint of a t(14; 18) translocation present in human B cell lymphomas [Tsujimoto, Y., et al. Science 228, 1440-1443 (1985); Cleary, M. L., et al Cell 47, 19-28 (1986); Boise, L. H. et al. Cell 74, 597-608 (1993)]. This protein has since been shown to have a dominant role in the survival of multiple lymphoid malignancies [Vaux, D. L., et al pre-B cells. Nature 335, 440-442 (1988)]. Overexpression of Bcl-2 proteins correlates with resistance to chemotherapy, clinical outcome, disease progression, overall prognosis or a combination thereof in various cancers and disorders of the immune system. Involvement of Bcl-2 proteins in bladder cancer, brain cancer, breast cancer, bone marrow cancer, cervical cancer, chronic lymphocytic leukemia, colorectal cancer, esophageal cancer, hepatocellular cancer, lymphoblastic leukemia, follicular lymphoma, lymphoid malignancies of T-cell or B-cell origin, melanoma, myelogenous leukemia, myeloma, oral cancer, ovarian cancer, non-small cell lung cancer, prostate cancer, small cell lung cancer, spleen cancer, and the like is described in PCT/US2004/36770, published as WO 2005/049593, and PCT/US2004/37911, published as WO/2005/049594. Involvement of Bcl-2 proteins in immune and autoimmune diseases is described in Current Allergy and Asthma Reports 2003, 3, 378-384; British Journal of Hematology 2000, 110(3), 584-90; Blood 2000, 95(4), 1283-92; and New England Journal of Medicine 2004, 351(14), 1409-1418. Involvement of Bcl-2 proteins in arthritis is disclosed in WO 2009/064938. Involvement of Bcl-2 proteins in bone marrow transplant rejection is disclosed in US 2008-0182845 A1. All incorporated herein by reference.

In the last decade, several Bcl-2 inhibitors such as ABT-737, ABT-263, and ABT-199 as shown below have been identified and entered human clinical trials for cancers treatment.

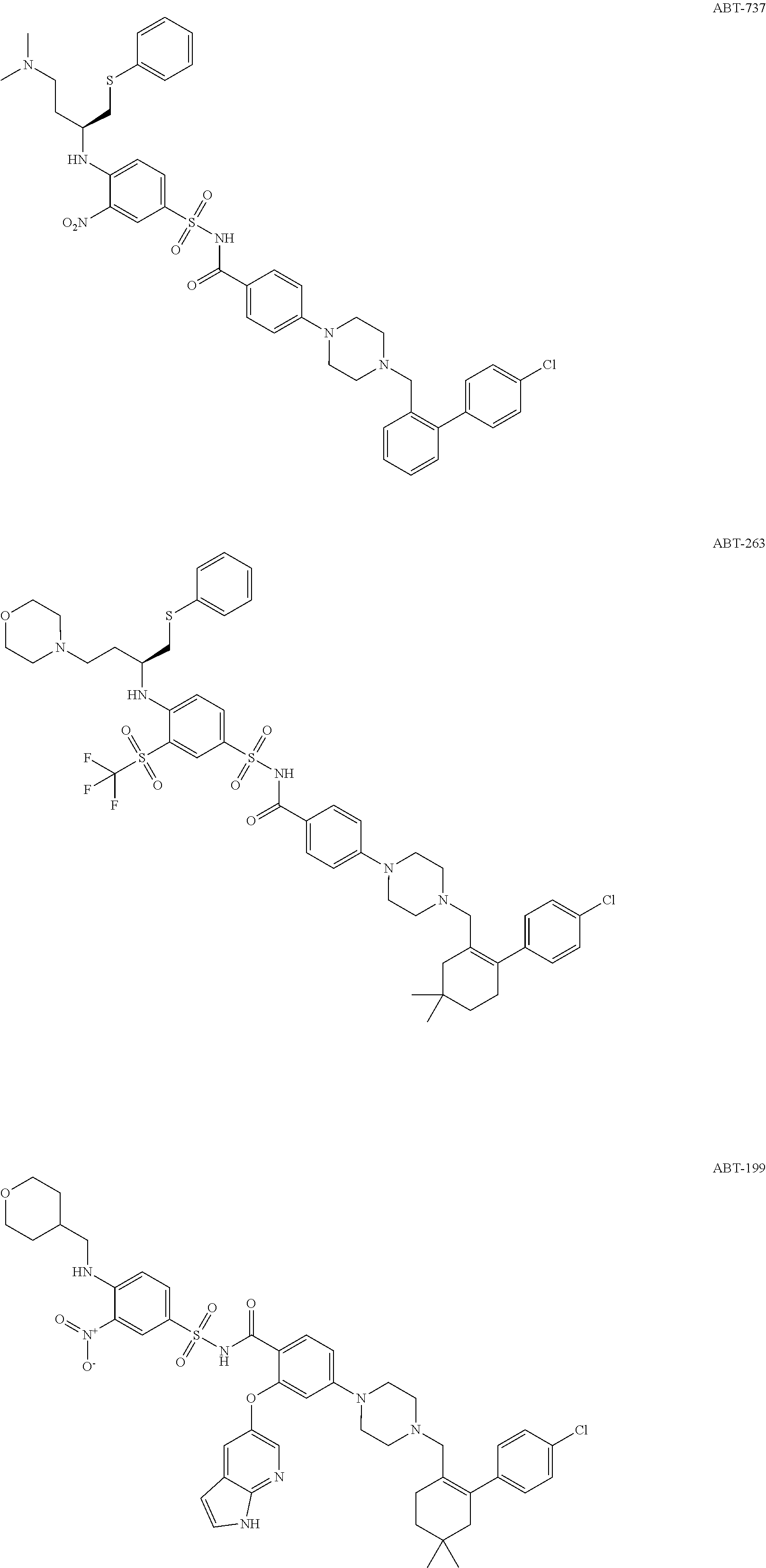

ABT-737 is discovered by nuclear magnetic resonance (NMR)-based screening, parallel synthesis and structure based fragment drug design [Tillman Oltersdorf, et al, Nature, Vol 435, 2005, p 677]. ABT-737 a small-molecule inhibitor of the anti-apoptotic proteins Bcl-2, Bcl-XL and Bcl-w, with an affinity two to three orders of magnitude more potent than previously reported compounds. Mechanistic studies reveal that ABT-737 does not directly initiate the apoptotic process, but enhances the effects of death signals, displaying synergistic cytotoxicity with chemotherapeutics and radiation. ABT-737 exhibits single-agent-mechanism-based killing of cells from lymphoma and small-cell lung carcinoma lines, as well as primary patient-derived cells, and in animal models, ABT-737 improves survival, causes regression of established tumors, and produces cures in a high percentage of the mice. Unfortunately, ABT-737 is not orally bioavailable, and its formulation for intravenous delivery is hampered by its low aqueous solubility.

After extensive MedChem effort, an orally bioavailable Bcl-2 inhibitor ABT-263 (Navitoclax) has been developed [Cheol-Min Park, et al J. Med. Chem. 2008, 51, 6902-6915]. ABT-263 is a potent inhibitor of Bcl-xL, Bcl-2 and Bcl-w with Ki of ≤0.5 nM, ≤1 nM and ≤1 nM. ABT-263 has an IC₅₀ of 110 nM against SCLC H146 cell line. When ABT-263 is administered at 100 mg/kg/day in the H345 xenograft model, significant antitumor efficacy is observed with 80% TGI and 20% of treated tumors indicating at least a 50% reduction in tumor volume. Oral administration of ABT-263 alone causes complete tumor regressions in xenograft models of small-cell lung cancer and acute lymphoblastic leukemia [Tse C, et al. Cancer Res. 2008, 68(9), 3421-3428]. In the clinical trial, however, the inhibition of BCL-XL by ABT-263 (navitoclax) induces a rapid, concentration-dependent decrease in the number of circulating platelets. This mechanism-based thrombocytopenia is the dose-limiting toxicity of single-agent navitoclax treatment in patients and limits the ability to drive drug concentrations into a highly efficacious range.

Thus, a BCL-2 selective (BCL-XL sparing) inhibitor would culminate in substantially reduced thrombocytopenia while maintaining efficacy in lymphoid malignancies. The resulting increase in the therapeutic window should allow for greater BCL-2 suppression and clinical efficacy in BCL-2-dependent tumor types. After extensive MedChem, ABT-199 (GDC-0199) has been successfully developed [Andrew J Souers, et al, Nature Medicine, Volume 19, 22, p 202, 2013]. ABT-199 is a Bcl-2-selective inhibitor with Ki of <0.01 nM, >4800-fold more selective versus Bcl-xL and Bcl-w, and no activity to Mcl-1. ABT-199 potently inhibits RS4; 11 cells with EC₅₀ of 8 nM. In addition, ABT-199 induces a rapid apoptosis in RS4; 11 cells with cytochrome c release, caspase activation, and the accumulation of sub-G0/G1 DNA. Quantitative immunoblotting reveals that sensitivity to ABT-199 correlated strongly with the expression of Bcl-2, including NHL, DLBCL, MCL, AML and ALL cell lines. ABT-199 also induces apoptosis in CLL with an average EC₅₀ of 3.0 nM. A single dose of 100 mg/kg of ABT-199 causes a maximal tumor growth inhibition of 95% and tumor growth delay of 152% in RS4; 11 xenografts. ABT-199 also inhibits xenograft growth (DoHH2, Granta-519) as a single agent or in combination with Bendamustine and other agents. Human Phase I and II data showed that ABT-199 is highly efficacious for CLL who have 17p deletion, and was approved by FDA in 2016.

WO/2017/132474, WO/2019/040550, and WO/2019/040573 disclosed a novel class of BCL-2 inhibitors. However, there is still a strong need for continuing search in this field of art for more potent BCL-2 inhibitor.

### SUMMARY OF THE INVENTION

In a first embodiment, this invention provides compounds of the Formula (1) or an N-oxide thereof, or a pharmaceutically acceptable salt, solvate, polymorph, tautomer, stereoisomer, an isotopic form, or a prodrug of said compound of Formula (1) or N-oxide thereof:

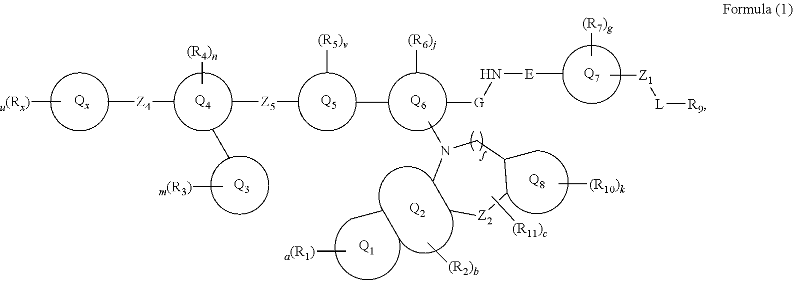

wherein
G is C(O), S(O₂), P(O)(Rₐ), or S(O)(NRₐ);
E is S(O₂), P(O)(Rₐ), S(O)(NRₐ) or C(O);
Q₁ is a cycloalkyl, cycloalkenyl, spirocycloalkyl, fused-carbocyclic, bridged-carbocyclic, heterocycloalkyl, heterocycloalkenyl, spiro-heterocyclic, fused-heterocyclic, bridged-heterocyclic, aryl, or heteroaryl, each of the aforementioned is optionally substituted with one or more independently selected R_{d} substituents;
Q₂ is a cycloalkyl, cycloalkenyl, spirocycloalkyl, fused-carbocyclic, bridged-carbocyclic, heterocycloalkyl, heterocycloalkenyl, spiro-heterocyclic, fused-heterocyclic, bridged-heterocyclic, aryl, or heteroaryl, each of the aforementioned is optionally substituted with one or more independently selected R_{d} substituents;
Q₃ is a cycloalkyl, cycloalkenyl, spirocycloalkyl, fused-carbocyclic, bridged-carbocyclic, heterocycloalkyl, heterocycloalkenyl, spiro-heterocyclic, fused-heterocyclic, bridged-heterocyclic, aryl, or heteroaryl, each of the aforementioned is optionally substituted with one or more independently selected R_{d} substituents;
Q₄ is a cycloalkyl, cycloalkenyl, spirocycloalkyl, fused-carbocyclic, bridged-carbocyclic, heterocycloalkyl, heterocycloalkenyl, spiro-heterocyclic, fused-heterocyclic, bridged-heterocyclic, aryl, or heteroaryl, each of the aforementioned is optionally substituted with one or more independently selected R_{d} substituents;
Q₅ is a cycloalkyl, cycloalkenyl, spirocycloalkyl, fused-carbocyclic, bridged-carbocyclic, heterocycloalkyl, heterocycloalkenyl, spiro-heterocyclic, fused-heterocyclic, bridged-heterocyclic, aryl, or heteroaryl, each of the aforementioned is optionally substituted with one or more independently selected R_{d} substituents;
Q₆ is a cycloalkyl, cycloalkenyl, spirocycloalkyl, fused-carbocyclic, bridged-carbocyclic, heterocycloalkyl, heterocycloalkenyl, spiro-heterocyclic, fused-heterocyclic, bridged-heterocyclic, aryl, or heteroaryl, each of the aforementioned is optionally substituted with one or more independently selected R_{d} substituents;
Q₇ is a cycloalkyl, cycloalkenyl, spirocycloalkyl, fused-carbocyclic, bridged-carbocyclic, heterocycloalkyl, heterocycloalkenyl, spiro-heterocyclic, fused-heterocyclic, bridged-heterocyclic, aryl, or heteroaryl, each of the aforementioned is optionally substituted with one or more independently selected R_{d} substituents;
Q₈ is a cycloalkyl, cycloalkenyl, spirocycloalkyl, fused-carbocyclic, bridged-carbocyclic, heterocycloalkyl, heterocycloalkenyl, spiro-heterocyclic, fused-heterocyclic, bridged-heterocyclic, aryl, or heteroaryl, each of the aforementioned is optionally substituted with one or more independently selected R_{d} substituents;
one or more (e.g., both) border ring atom(s) between ring Q₈ and the ring with Z₂ can be carbon or heteroatom(s); Qₓ is a cycloalkyl, cycloalkenyl, spirocycloalkyl, fused-carbocyclic, bridged-carbocyclic,
heterocycloalkyl, heterocycloalkenyl, spiro-heterocyclic, fused-heterocyclic, bridged-heterocyclic, aryl, or heteroaryl, each of the aforementioned is optionally substituted with one or more independently selected R_{d} substituents;
each R₁ is independently H, D, halo, cyano, nitro, alkyl, alkylene-Rₐ, alkylene-P(O)R_{b}R_{c}, alkenyl, alkynyl, —C(O)Rₐ, —C(O)NR_{b}R_{c}, —C(O)ORₐ, —NH(CH₂)ₚRₐ, —NR_{b}R_{c}, —NR_{b}C(O)R_{c}, =NR_{b}, —NR_{b}S(O)₂R_{c}, —N═S(O)R_{b}R_{c}, —ORₐ, —OC(O)Rₐ, ═O, —P(O)R_{b}R_{c}, —SRₐ, —S(O)Rₐ, —S(O)(NR_{b})R_{c}, —S(O)₂Rₐ, —S(O)₂NR_{b}R_{c}, cycloalkyl, cycloalkenyl, spirocycloalkyl, fused-carbocyclic, bridged-carbocyclic, heterocycloalkyl, heterocycloalkenyl, spiro-heterocyclic, fused-heterocyclic, bridged-heterocyclic, aryl, or heteroaryl, each of the aforementioned is optionally substituted with one or more independently selected R_{d} substituents;
each R₂ is independently H, D, halo, cyano, nitro, -alkyl, -alkylene-Rₐ, alkylene-P(O)R_{b}R_{c}, alkenyl, alkynyl, —C(O)Rₐ, —C(O)NR_{b}R_{c}, —C(O)ORₐ, —NH(CH₂)ₚRₐ, —NR_{b}R_{c}, —NR_{b}C(O)R_{c}, =NR_{b}, —NR_{b}S(O)₂R_{c}, —N═S(O)R_{b}R_{c}, —ORₐ, —OC(O)Rₐ, ═O, —P(O)R_{b}R_{c}, —SRₐ, —S(O)Rₐ, —S(O)(NR_{b})R_{c}, —S(O)₂Rₐ, —S(O)₂NR_{b}R_{c}, cycloalkyl, cycloalkenyl, spirocycloalkyl, fused-carbocyclic, bridged-carbocyclic, heterocycloalkyl, heterocycloalkenyl, spiro-heterocyclic, fused-heterocyclic, bridged-heterocyclic, aryl, or heteroaryl, each of the aforementioned is optionally substituted with one or more independently selected R_{d} substituents;
each R₃ is independently H, D, halo, cyano, nitro, alkyl, alkylene-Rₐ, alkylene-P(O)R_{b}R_{c}, alkenyl, alkynyl, —C(O)Rₐ, —C(O)NR_{b}R_{c}, —C(O)ORₐ, —NH(CH₂)ₚRₐ, —NR_{b}R_{c}, —NR_{b}C(O)R_{c}, =NR_{b}, —NR_{b}S(O)₂R_{c}, —N═S(O)R_{b}R_{c}, —ORₐ, —OC(O)Rₐ, ═O, —P(O)R_{b}R_{c}, —SRₐ, —S(O)Rₐ, —S(O)(NR_{b})R_{c}, —S(O)₂Rₐ, —S(O)₂NR_{b}R_{c}, cycloalkyl, cycloalkenyl, spirocycloalkyl, fused-carbocyclic, bridged-carbocyclic, heterocycloalkyl, heterocycloalkenyl, spiro-heterocyclic, fused-heterocyclic, bridged-heterocyclic, aryl, or heteroaryl, each of the aforementioned is optionally substituted with one or more independently selected R_{d} substituents; or
two R₃, taken together with the atom(s) to which they are attached, independently form a cycloalkyl or heterocycloalkyl, wherein each cycloalkyl or heterocycloalkyl is optionally and independently substituted with one or more independently selected R_{d} substituents;
each R₄ is independently H, D, halo, cyano, nitro, alkyl, alkylene-Rₐ, alkylene-P(O)R_{b}R_{c}, alkenyl, alkynyl, —C(O)Rₐ, —C(O)NR_{b}R_{c}, —C(O)ORₐ, —NH(CH₂)ₚRₐ, —NR_{b}R_{c}, —NR_{b}C(O)R_{c}, =NR_{b}, —NR_{b}S(O)₂R_{c}, —N═S(O)R_{b}R_{c}, —ORₐ, —OC(O)Rₐ, ═O, —P(O)R_{b}R_{c}, —SRₐ, —S(O)Rₐ, —S(O)(NR_{b})R_{c}, —S(O)₂Rₐ, —S(O)₂NR_{b}R_{c}, cycloalkyl, cycloalkenyl, spirocycloalkyl, fused-carbocyclic, bridged-carbocyclic, heterocycloalkyl, heterocycloalkenyl, spiro-heterocyclic, fused-heterocyclic, bridged-heterocyclic, aryl, or heteroaryl, each of the aforementioned is optionally substituted with one or more independently selected R_{d} substituents; or
two R₄, taken together with the atom(s) to which they are attached, independently form a cycloalkyl or heterocycloalkyl, wherein each cycloalkyl or heterocycloalkyl is optionally and independently substituted with one or more independently selected R_{d} substituents;
R₃ and R₄, taken together with the atom(s) to which they are attached, independently form a cycloalkyl or heterocycloalkyl, wherein each cycloalkyl or heterocycloalkyl is optionally and independently substituted with one or more independently selected R_{d} substituents;
each R₅ is independently H, D, halo, cyano, nitro, alkyl, alkylene-Rₐ, alkylene-P(O)R_{b}R_{c}, alkenyl, alkynyl, —C(O)Rₐ, —C(O)NR_{b}R_{c}, —C(O)ORₐ, —NH(CH₂)ₚRₐ, —NR_{b}R_{c}, —NR_{b}C(O)R_{c}, =NR_{b}, —NR_{b}S(O)₂R_{c}, —N═S(O)R_{b}R_{c}, —ORₐ, —OC(O)Rₐ, ═O, —P(O)R_{b}R_{c}, —SRₐ, —S(O)Rₐ, —S(O)(NR_{b})R_{c}, —S(O)₂Rₐ, —S(O)₂NR_{b}R_{c}, cycloalkyl, cycloalkenyl, spirocycloalkyl, fused-carbocyclic, bridged-carbocyclic, heterocycloalkyl, heterocycloalkenyl, spiro-heterocyclic, fused-heterocyclic, bridged-heterocyclic, aryl, or heteroaryl, each of the aforementioned is optionally substituted with one or more independently selected R_{d} substituents; or
R₄ and R₅, taken together with the atom(s) to which they are attached, independently form a cycloalkyl or heterocycloalkyl, wherein each cycloalkyl or heterocycloalkyl is optionally and independently substituted with one or more independently selected R_{d} substituents;
each R₆ is independently H, D, halo, cyano, nitro, alkyl, alkylene-Rₐ, alkylene-P(O)R_{b}R_{c}, alkenyl, alkynyl, —C(O)Rₐ, —C(O)NR_{b}R_{c}, —C(O)ORₐ, —NH(CH₂)ₚRₐ, —NR_{b}R_{c}, —NR_{b}C(O)R_{c}, =NR_{b}, —NR_{b}S(O)₂R_{c}, —N═S(O)R_{b}R_{c}, —ORₐ, —OC(O)Rₐ, ═O, —P(O)R_{b}R_{c}, —SRₐ, —S(O)Rₐ, —S(O)(NR_{b})R_{c}, —S(O)₂Rₐ, —S(O)₂NR_{b}R_{c}, cycloalkyl, cycloalkenyl, spirocycloalkyl, fused-carbocyclic, bridged-carbocyclic, heterocycloalkyl, heterocycloalkenyl, spiro-heterocyclic, fused-heterocyclic, bridged-heterocyclic, aryl, or heteroaryl, each of the aforementioned is optionally substituted with one or more independently selected R_{d} substituents; or
R₅ and R₆, taken together with the atom(s) to which they are attached, independently form a cycloalkyl or heterocycloalkyl, wherein each cycloalkyl or heterocycloalkyl is optionally and independently substituted with one or more independently selected R_{d} substituents;
each R₇ is independently H, D, halo, cyano, nitro, alkyl, alkylene-Rₐ, alkylene-P(O)R_{b}R_{c}, alkenyl, alkynyl, —C(O)Rₐ, —C(O)NR_{b}R_{c}, —C(O)ORₐ, —NH(CH₂)ₚRₐ, —NR_{b}R_{c}, —NR_{b}C(O)R_{c}, =NR_{b}, —NR_{b}S(O)₂R_{c}, —N═S(O)R_{b}R_{c}, —ORₐ, —OC(O)Rₐ, ═O, —P(O)R_{b}R_{c}, —SRₐ, —S(O)Rₐ, —S(O)(NR_{b})R_{c}, —S(O)₂Rₐ, —S(O)₂NR_{b}R_{c}, cycloalkyl, cycloalkenyl, spirocycloalkyl, fused-carbocyclic, bridged-carbocyclic, heterocycloalkyl, heterocycloalkenyl, spiro-heterocyclic, fused-heterocyclic, bridged-heterocyclic, aryl, or heteroaryl, each of the aforementioned is optionally substituted with one or more independently selected R_{d} substituents; or
R₇ and —Z₁-L-R₉, taken together with the atom(s) to which they are attached, independently form a cycloalkyl or heterocycloalkyl, wherein each cycloalkyl or heterocycloalkyl is optionally and independently substituted with one or more independently selected R_{d} substituents;
R₉ is H, D, halo, cyano, nitro, alkyl, alkylene-Rₐ, alkylene-P(O)R_{b}R_{c}, alkenyl, alkynyl, —C(O)Rₐ, —C(O)NR_{b}R_{c}, —C(O)ORₐ, —NH(CH₂)ₚRₐ, —NR_{b}R_{c}, —NR_{b}C(O)R_{c}, =NR_{b}, —NR_{b}S(O)₂Rₐ, —N═S(O)R_{b}R_{c}, —ORₐ, —OC(O)Rₐ, ═O, —P(O)R_{b}R_{c}, —SRₐ, —S(O)Rₐ, —S(O)(NR_{b})R_{c}, —S(O)₂Rₐ, —S(O)₂NR_{b}R_{c}, cycloalkyl, cycloalkenyl, spirocycloalkyl, fused-carbocyclic, bridged-carbocyclic, heterocycloalkyl, heterocycloalkenyl, spiro-heterocyclic, fused-heterocyclic, bridged-heterocyclic, aryl, or heteroaryl, each of the aforementioned is optionally substituted with one or more independently selected R_{d} substituents;
each R₁₀ is independently H, D, halo, cyano, nitro, alkyl, alkylene-Rₐ, alkylene-P(O)R_{b}R_{c}, alkenyl, alkynyl, —C(O)Rₐ, —C(O)NR_{b}R_{c}, —C(O)ORₐ, —NH(CH₂)ₚRₐ, —NR_{b}R_{c}, —NR_{b}C(O)R_{c}, =NR_{b}, —NR_{b}S(O)₂R_{c}, —N═S(O)R_{b}R_{c}, —ORₐ, —OC(O)Rₐ, ═O, —P(O)R_{b}R_{c}, —SRₐ, —S(O)Rₐ, —S(O)(NR_{b})R_{c}, —S(O)₂Rₐ, —S(O)₂NR_{b}R_{c}, cycloalkyl, cycloalkenyl, spirocycloalkyl, fused-carbocyclic, bridged-carbocyclic, heterocycloalkyl, heterocycloalkenyl, spiro-heterocyclic, fused-heterocyclic, bridged-heterocyclic, aryl, or heteroaryl, each of the aforementioned is optionally substituted with one or more independently selected R_{d} substituents; or
two R₁₀, taken together with the atom(s) to which they are attached, independently form a cycloalkyl or heterocycloalkyl, wherein each cycloalkyl or heterocycloalkyl is optionally and independently substituted with one or more independently selected R_{d} substituents;
R₁₁ is H, D, halo, cyano, nitro, alkyl, alkylene-Rₐ, alkylene-P(O)R_{b}R_{c}, alkenyl, alkynyl, —C(O)Rₐ, —C(O)NR_{b}R_{c}, —C(O)ORₐ, —NH(CH₂)ₚRₐ, —NR_{b}R_{c}, —NR_{b}C(O)R_{c}, =NR_{b}, —NR_{b}S(O)₂Rₐ, —N═S(O)R_{b}R_{c}, —ORₐ, —OC(O)Rₐ, ═O, —P(O)R_{b}R_{c}, —SRₐ, —S(O)Rₐ, —S(O)(NR_{b})R_{c}, —S(O)₂Rₐ, —S(O)₂NR_{b}R_{c}, cycloalkyl, cycloalkenyl, spirocycloalkyl, fused-carbocyclic, bridged-carbocyclic, heterocycloalkyl, heterocycloalkenyl, spiro-heterocyclic, fused-heterocyclic, bridged-heterocyclic, aryl, or heteroaryl, each of the aforementioned is optionally substituted with one or more independently selected R_{d} substituents; or
R₁₀ and R₁₁, taken together with the atom(s) to which they are attached, independently form a cycloalkyl or heterocycloalkyl, wherein each cycloalkyl or heterocycloalkyl is optionally and independently substituted with one or more independently selected R_{d} substituents;
each Rₓ is independently H, D, halo, cyano, nitro, alkyl, alkylene-Rₐ, alkylene-P(O)R_{b}R_{c}, alkenyl, alkynyl, —C(O)Rₐ, —C(O)NR_{b}R_{c}, —C(O)ORₐ, —NH(CH₂)ₚRₐ, —NR_{b}R_{c}, —NR_{b}C(O)R_{c}, =NR_{b}, —NR_{b}S(O)₂R_{c}, —N═S(O)R_{b}R_{c}, —ORₐ, —OC(O)Rₐ, ═O, —P(O)R_{b}R_{c}, —SRₐ, —S(O)Rₐ, —S(O)(NR_{b})R_{c}, —S(O)₂Rₐ, —S(O)₂NR_{b}R_{c}, cycloalkyl, cycloalkenyl, spirocycloalkyl, fused-carbocyclic, bridged-carbocyclic, heterocycloalkyl, heterocycloalkenyl, spiro-heterocyclic, fused-heterocyclic, bridged-heterocyclic, aryl, or heteroaryl, each of the aforementioned is optionally substituted with one or more independently selected R_{d} substituents; or
two Rₓ, taken together with the atom(s) to which they are attached, independently form a cycloalkyl or heterocycloalkyl, wherein each cycloalkyl or heterocycloalkyl is optionally and independently substituted with one or more independently selected R_{d} substituents;
R₄ and Rₓ, taken together with the atom(s) to which they are attached, independently form a cycloalkyl or heterocycloalkyl, wherein each cycloalkyl or heterocycloalkyl is optionally and independently substituted with one or more independently selected R_{d} substituents;
Z₁ is a bond, (CRₐR_{b})ₚ, a bivalent alkenyl group, or a bivalent alkynyl group, N(Rₐ), O, S, C(O), S(O₂), —O(CRₐR_{b})ₚ—, —N(Rₐ)(CRₐR_{b})ₚ—, OC(O), C(O)O, OSO₂, S(O₂)O, C(O)S, SC(O), C(O)C(O), C(O)N(Rₐ), N(Rₐ)C(O), S(O₂)N(Rₐ), N(Rₐ)S(O₂), OC(O)O, OC(O)S, OC(O)N(Rₐ), N(Rₐ)C(O)O, N(Rₐ)C(O)S, N(Rₐ)C(O)N(Rₐ), (CRₐR_{b})ₚN(Rₐ)(CRₐR_{b})_{q}, (CRₐR_{b})ₚN(Rₐ)C(O)(CRₐR_{b})_{q}, OC(O)N(R_{b})(CRₐR_{b})ₚ₊₁N(R_{b})(CRₐR_{b})_{q}, or (CRₐR_{b})ₚC(O)N(Rₐ)(CRₐR_{b})_{q};
Z₂ is a bond, (CRₐR_{b})ₚ, a bivalent alkenyl group, or a bivalent alkynyl group, N(Rₐ), O, S, C(O), S(O₂), —O(CRₐR_{b})ₚ—, —N(Rₐ)(CRₐR_{b})ₚ—, OC(O), C(O)O, OSO₂, S(O₂)O, C(O)S, SC(O), C(O)C(O), C(O)N(Rₐ), N(Rₐ)C(O), S(O₂)N(Rₐ), N(Rₐ)S(O₂), OC(O)O, OC(O)S, OC(O)N(Rₐ), N(Rₐ)C(O)O, N(Rₐ)C(O)S, N(Rₐ)C(O)N(Rₐ), (CRₐR_{b})ₚN(Rₐ)(CRₐR_{b})_{q}, (CRₐR_{b})ₚN(Rₐ)C(O)(CRₐR_{b})_{q}, OC(O)N(R_{b})(CRₐR_{b})ₚ₊₁N(R_{b})(CRₐR_{b})_{q}, or (CRₐR_{b})ₚC(O)N(Rₐ)(CRₐR_{b})_{q};
Z₄ is a bond, (CRₐR_{b})ₚ, a bivalent alkenyl group, or a bivalent alkynyl group, N(Rₐ), O, S, C(O), S(O₂), —O(CRₐR_{b})ₚ—, —N(Rₐ)(CRₐR_{b})ₚ—, OC(O), C(O)O, OSO₂, S(O₂)O, C(O)S, SC(O), C(O)C(O), C(O)N(Rₐ), N(Rₐ)C(O), S(O₂)N(Rₐ), N(Rₐ)S(O₂), OC(O)O, OC(O)S, OC(O)N(Rₐ), N(Rₐ)C(O)O, N(Rₐ)C(O)S, N(Rₐ)C(O)N(Rₐ), (CRₐR_{b})ₚN(Rₐ)(CRₐR_{b})_{q}, (CRₐR_{b})ₚN(Rₐ)C(O)(CRₐR_{b})_{q}, OC(O)N(R_{b})(CRₐR_{b})ₚ₊₁N(R_{b})(CRₐR_{b})_{q}, or (CRₐR_{b})ₚC(O)N(Rₐ)(CRₐR_{b})_{q};
Z₅ is a bond, (CRₐR_{b})ₚ, a bivalent alkenyl group, or a bivalent alkynyl group, N(Rₐ), O, S, C(O), S(O₂), —O(CRₐR_{b})ₚ—, —N(Rₐ)(CRₐR_{b})ₚ—, OC(O), C(O)O, OSO₂, S(O₂)O, C(O)S, SC(O), C(O)C(O), C(O)N(Rₐ), N(Rₐ)C(O), S(O₂)N(Rₐ), N(Rₐ)S(O₂), OC(O)O, OC(O)S, OC(O)N(Rₐ), N(Rₐ)C(O)O, N(Rₐ)C(O)S, N(Rₐ)C(O)N(Rₐ), (CRₐR_{b})ₚN(Rₐ)(CRₐR_{b})_{q}, (CRₐR_{b})ₚN(Rₐ)C(O)(CRₐR_{b})_{q}, OC(O)N(R_{b})(CRₐR_{b})ₚ₊₁N(R_{b})(CRₐR_{b})_{q}, or (CRₐR_{b})ₚC(O)N(Rₐ)(CRₐR_{b})_{q};
L is absent, a bond, (CRₐR_{b})ₚ, N(R_{c}), O, S, C(O), S(O₂), —O(CRₐR_{b})ₚ—, —N(R_{c})(CRₐR_{b})ₚ—, OC(O), C(O)O, OSO₂, S(O₂)O, C(O)S, SC(O), C(O)C(O), C(O)N(R_{c}), N(R_{c})C(O), S(O₂)N(R_{c}), N(R_{c})S(O₂), OC(O)O, OC(O)S, OC(O)N(R_{c}), N(R_{c})C(O)O, N(R_{c})C(O)S, N(R_{c})C(O)N(R_{c}), (CRₐR_{b})ₚN(R_{c})(CRₐR_{b})_{q}, (CRₐR_{b})ₚN(R_{c})C(O)(CRₐR_{b})_{q}, OC(O)N(R_{c})(CRₐR_{b})ₚ₊₁N(R_{c})(CRₐR_{b})_{q}, (CRₐR_{b})ₚC(O)N(R_{c})(CRₐR_{b})_{q}, bivalent alkenyl, bivalent alkynyl, bivalent cycloalkyl, bivalent cycloalkenyl, bivalent spirocycloalkyl, bivalent fused-carbocyclic, bivalent bridged-carbocyclic, bivalent heterocycloalkyl, bivalent heterocycloalkenyl, bivalent spiro-heterocyclic, bivalent fused-heterocyclic, bivalent bridged-heterocyclic, bivalent aryl, or bivalent heteroaryl, each of the aforementioned is independently optionally substituted with one or more R_{d};
each Rₐ is independently H, D, halo, cyano, nitro, alkyl, alkylene-P(O)RₑR_{f}, alkenyl, alkynyl, —C(O)alkyl, —C(O)NHOH, —C(O)NH₂, —C(O)OH, —C(O)O(alkyl), —NH₂, —NH(alkyl), —NH(haloalkyl), —NRₑR_{f}, —NHC(O)alkyl, =NRₑ, —N═S(O)RₑR_{f}, —OH, —O(alkyl), ═O, —P(O)RₑR_{f}, —S(O)(NRₑ)R_{f}, cycloalkyl, cycloalkenyl, spirocycloalkyl, fused-carbocyclic, bridged-carbocyclic, heterocycloalkyl, heterocycloalkenyl, spiro-heterocyclic, fused-heterocyclic, bridged-heterocyclic, aryl, or heteroaryl, each of the aforementioned is optionally and independently substituted with one or more independently selected Rₑ substituents;
each R_{b} is independently H, D, halo, cyano, nitro, alkyl, alkylene-P(O)RₑR_{f}, alkenyl, alkynyl, —C(O)alkyl, —C(O)NHOH, —C(O)NH₂, —C(O)OH, —C(O)O(alkyl), —NH₂, —NH(alkyl), —NH(haloalkyl), —NRₑR_{f}, —NHC(O)alkyl, =NRₑ, —N═S(O)RₑR_{f}, —OH, —O(alkyl), ═O, —P(O)RₑR_{f}, —S(O)(NRₑ)R_{f}, cycloalkyl, cycloalkenyl, spirocycloalkyl, fused-carbocyclic, bridged-carbocyclic, heterocycloalkyl, heterocycloalkenyl, spiro-heterocyclic, fused-heterocyclic, bridged-heterocyclic, aryl, or heteroaryl, each of the aforementioned is optionally and independently substituted with one or more independently selected Rₑ substituents;
each R_{c} is independently H, D, halo, cyano, nitro, alkyl, alkylene-P(O)RₑR_{f}, alkenyl, alkynyl, —C(O)alkyl, —C(O)NHOH, —C(O)NH₂, —C(O)OH, —C(O)O(alkyl), —NH₂, —NH(alkyl), —NH(haloalkyl), —NRₑR_{f}, —NHC(O)alkyl, =NRₑ, —N═S(O)RₑR_{f}, —OH, —O(alkyl), ═O, —P(O)RₑR_{f}, —S(O)(NRₑ)R_{f}, cycloalkyl, cycloalkenyl, spirocycloalkyl, fused-carbocyclic, bridged-carbocyclic, heterocycloalkyl, heterocycloalkenyl, spiro-heterocyclic, fused-heterocyclic, bridged-heterocyclic, aryl, or heteroaryl, each of the aforementioned is optionally and independently substituted with one or more independently selected Rₑ substituents;
R_{b} and R_{c}, taken together with the atom(s) to which they are attached, independently form a cycloalkyl or heterocycloalkyl, wherein each cycloalkyl or heterocycloalkyl is optionally and independently substituted with one or more independently selected Rₑ substituents;
each R_{d} is independently H, D, halo, cyano, nitro, alkyl, alkylene-P(O)RₑR_{f}, alkenyl, alkynyl, —C(O)alkyl, —C(O)NHOH, —C(O)NH₂, —C(O)OH, —C(O)O(alkyl), —NH₂, —NH(alkyl), —NH(haloalkyl), —NRₑR_{f}, —NHC(O)alkyl, =NRₑ, —N═S(O)RₑR_{f}, —OH, —O(alkyl), ═O, —P(O)RₑR_{f}, —S(O)(NRₑ)R_{f}, cycloalkyl, cycloalkenyl, spirocycloalkyl, fused-carbocyclic, bridged-carbocyclic, heterocycloalkyl, heterocycloalkenyl, spiro-heterocyclic, fused-heterocyclic, bridged-heterocyclic, aryl, or heteroaryl, each of the aforementioned is optionally and independently substituted with one or more independently selected Rₑ substituents; or
two R_{d}, taken together with the atom(s) to which they are attached, independently form a cycloalkyl or heterocycloalkyl, wherein each cycloalkyl or heterocycloalkyl is optionally and independently substituted with one or more independently selected Rₑ substituents;
each Rₑ is independently H, D, halo, cyano, nitro, alkyl, alkylene, alkenyl, alkynyl, —C(O)alkyl, —C(O)NHOH, —C(O)NH₂, —C(O)OH, —C(O)O(alkyl), —NH₂, —NH(alkyl), —N(alkyl)(alkyl), —NH(haloalkyl), —NHC(O)alkyl, =N(alkyl), —N═S(O) (alkyl)(alkyl), —OH, —O(alkyl), =O, cycloalkyl, cycloalkenyl, spirocycloalkyl, fused-carbocyclic, bridged-carbocyclic, heterocycloalkyl, heterocycloalkenyl, spiro-heterocyclic, fused-heterocyclic, bridged-heterocyclic, aryl, or heteroaryl, each of the aforementioned is optionally and independently substituted with one or more independently selected R_{f} substituents; or
two Rₑ, taken together with the atom(s) to which they are attached, independently form a cycloalkyl or heterocycloalkyl, wherein each cycloalkyl or heterocycloalkyl is optionally and independently substituted with one or more independently selected R_{f} substituents;
each R_{f} is independently H, D, halo, cyano, nitro, alkyl, alkylene, alkenyl, alkynyl, —C(O)alkyl, —C(O)NHOH, —C(O)NH₂, —C(O)OH, —C(O)O(alkyl), —NH₂, —NH(alkyl), —N(alkyl)(alkyl), —NH(haloalkyl), —NHC(O)alkyl, =N(alkyl), —N═S(O) (alkyl)(alkyl), —OH, —O(alkyl), =O, cycloalkyl, cycloalkenyl, spirocycloalkyl, fused-carbocyclic, bridged-carbocyclic, heterocycloalkyl, heterocycloalkenyl, spiro-heterocyclic, fused-heterocyclic, bridged-heterocyclic, aryl, or heteroaryl, each of the aforementioned is optionally and independently substituted with one or more independently selected R_{g} substituents;
Rₑ and R_{f}, taken together with the atom(s) to which they are attached, independently form a cycloalkyl or heterocycloalkyl, wherein each cycloalkyl or heterocycloalkyl is optionally and independently substituted with one or more independently selected R_{g} substituents;
each R_{g} is independently H, D, halo, cyano, nitro, alkyl, alkylene, alkenyl, alkynyl, —C(O)alkyl, —C(O)NHOH, —C(O)NH₂, —C(O)OH, —C(O)O(alkyl), —NH₂, —NH(alkyl), —N(alkyl)(alkyl), —NH(haloalkyl), NHC(O)alkyl, =N(alkyl), —N═S(O) (alkyl)(alkyl), —OH, —O(alkyl), =O, cycloalkyl, cycloalkenyl, spirocycloalkyl, fused-carbocyclic, bridged-carbocyclic, heterocycloalkyl, heterocycloalkenyl, spiro-heterocyclic, fused-heterocyclic, bridged-heterocyclic, aryl, or heteroaryl;
a is 0, 1, 2, 3, 4, 5, 6, 7, or 8;
b is 0, 1, 2, 3, 4, 5, 6, 7, or 8;
c is 0, 1, 2, 3, 4, 5, 6, 7, or 8;
g is 0, 1, 2, 3, 4, 5, 6, 7, or 8;
j is 0, 1, 2, 3, 4, 5, 6, 7, or 8;
k is 0, 1, 2, 3, 4, 5, 6, 7, or 8;
m is 0, 1, 2, 3, 4, 5, 6, 7, or 8;
n is 0, 1, 2, 3, 4, 5, 6, 7, or 8;
v is 0, 1, 2, 3, 4, 5, 6, 7, or 8;
u is 0, 1, 2, 3, 4, or 5;
f is 0, 1, or 2;
p is 0, 1, 2, 3, 4, or 5; and
q is 0, 1, 2, 3, 4, or 5.

In certain embodiments, the invention provides a compound represented by Formula (2):

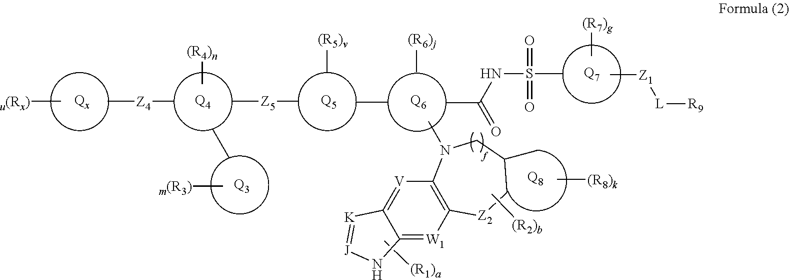

wherein
each of W₁, V, K, J, independently, is C(Rₐ) or N.

In certain embodiments, the invention provides a compound represented by Formula (3):

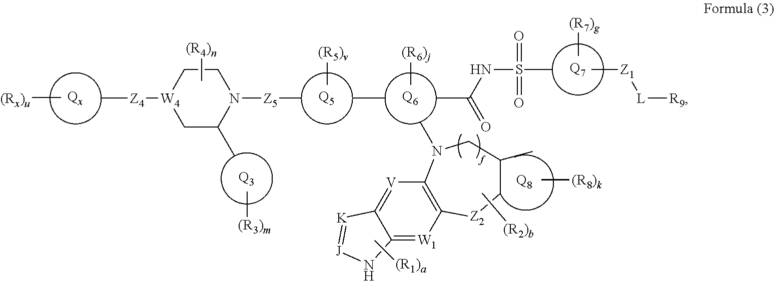

wherein
W₄ is C(Rₐ) or N.

In certain embodiments, the invention provides a compound represented by Formula (4):

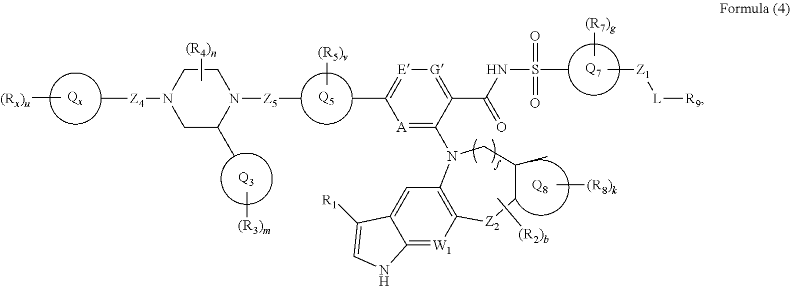

wherein
each of A, E′, and G′, independently is C(Rₐ) or N.

In certain embodiments, the invention provides a compound represented by Formula (5):

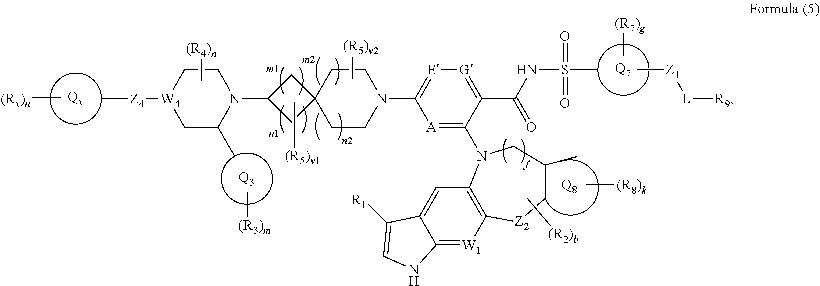

wherein
each of m1, m2, n1, and n2, independently, is 0, 1, or 2; and
v1 and v2 are both integers including 0, and v1+v2=v.

In certain embodiments, the invention provides a compound represented by Formula (6):

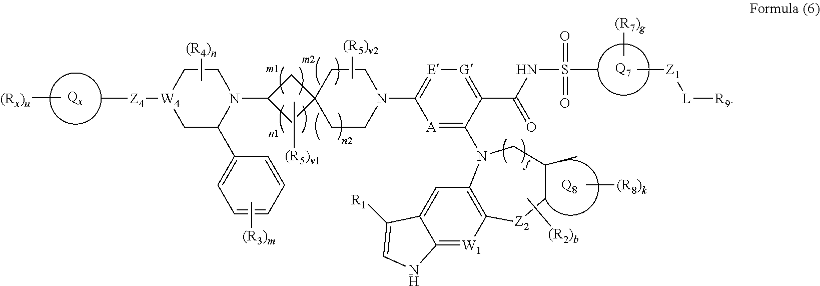

In certain embodiments, the invention provides a compound represented by Formula (7):

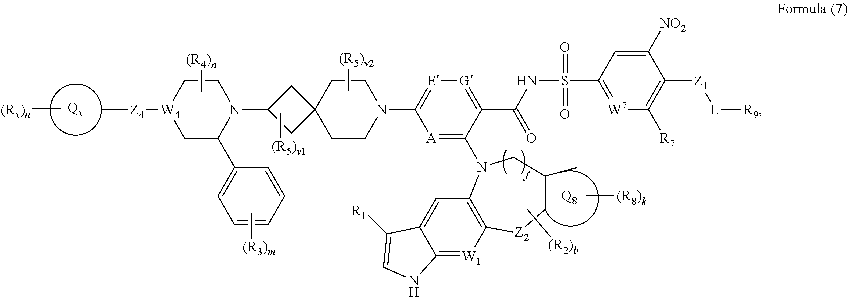

wherein
W⁷ is N, or C(Rₐ).

In certain embodiments, the invention provides a compound represented by Formula (8):

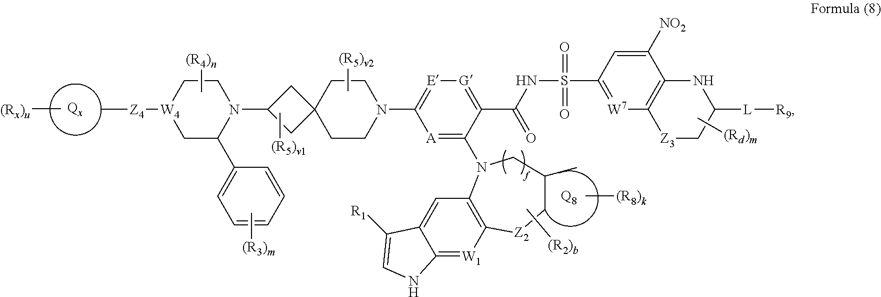

wherein
W⁷ is N, or C(Rₐ); and
Z₃ is a bond, (CH₂)ₚ, N(H), O, S, C(O), S(O₂), OC(O), C(O)O, OSO₂, S(O₂)O, C(O)S, SC(O), C(O)C(O), C(O)N(H), N(H)C(O), S(O₂)N(H), N(H)S(O₂), OC(O)O, OC(O)S, OC(O)N(H), N(H)C(O)O, N(H)C(O)S, N(H)C(O)N(H), (CH₂)ₚN(H)(CH₂)_{q}, (CH₂)ₚN(H)C(O)(CH₂)_{q}, (CH₂)ₚC(O)N(H)(CH₂)_{q}, or OC(O)N(H)(CH₂)ₚ₊₁N(H)(CH₂)_{q}.

A modified compound of any one of such compounds including a modification having an improved (e.g., enhanced, greater) pharmaceutical solubility, stability, bioavailability, and/or therapeutic index as compared to the unmodified compound is also contemplated. Exemplary modifications include (but are not limited to) applicable prodrug derivatives, and deuterium-enriched compounds.

Also within the scope of this invention is a pharmaceutical composition containing one or more of the compounds (such as any one of those in Formulae (1-7), (A-E), (I-VIII), or a pharmaceutically acceptable salt, solvate, polymorph, tautomer, stereoisomer, an isotopic form, or a prodrug thereof or an N-oxide thereof), modifications, and/or salts thereof described herein, and a pharmaceutically acceptable diluent or carrier, for use in treating a neoplastic disease, therapeutic uses thereof, and use of the compounds for the manufacture of a medicament for treating the disease/disorder.

This invention also relates to a method of treating a neoplastic disease, an autoimmune disease, or a neurodegenerative disease, comprising administering to a subject in need thereof an effective amount of one or more compounds of the invention (such as any one of those in Formulae (1-7), (A-E), (I-VIII), or a pharmaceutically acceptable salt, solvate, polymorph, tautomer, stereoisomer, an isotopic form, or a prodrug thereof or an N-oxide thereof), modifications, and/or salts thereof described herein, or a pharmaceutical composition comprising the compound(s) of the invention.

In certain embodiments, the neoplastic disease, autoimmune disease, or neurodegenerative disease is characterized by abnormal (e.g., enhanced or increased) Bcl-2 activity. For example, the neoplastic disease can be a hematological malignancy or cancer including solid tumor; the autoimmune disease can be type I diabetes; and the neurodegenerative disease can be schizophrenia.

In certain embodiments, the neoplastic disease is myeloma, multiple myeloma, lymphoma, follicular lymphoma (FL), non-Hodgkin's lymphoma, leukemia, acute leukemia, acute lymphoblastic leukemia (ALL) (such as BCL-2-dependent ALL and pediatric ALL), chronic lymphoblastic leukemia (CLL) (such as relapsed/refractory CLL, del(17p) CLL), chronic myeloid leukemia (CML) (such as blast-crisis CML), mantle cell lymphoma (MCL), diffuse large B-cell lymphoma, lung cancer such as small cell lung cancer (SCLC), melanoma, breast cancer, or prostate cancer, including drug-resistant cancer thereof.

In certain embodiments, the method further comprises administering one or more further treatment(s) effective to treat the neoplastic disease, such as surgery, radiation therapy, a chemotherapeutic agent (such as bendamustine, NL-101 (7-(5-(bis(2-chloroethyl)amino)-1-methyl-1H-benzo[d]imidazol-2-yl)-N-hydroxyheptanamide), cisplatin, carboplatin, etoposide, topotecan), a target therapy (e.g., an anti-CD20 antibody such as rituximab, a Bruton's tyrosine kinase inhibitor such as ibrutinib and acalabrutinib (ACP-196), a PI3Kδ inhibitor such as idelalisib); an antibody-drug conjugate or ADC (such as anti-CD30 ADC brentuximab vedotin), an immunotherapy (such as an anti-PD-1 antibody including pembrolizumab and nivolumab, or an anti-PD-L1 antibody including atezolizumab, durvalumab, and avelumab), or a CAR-T therapy (such as tisagenlecleucel, axicabtagene ciloleucel).

Also provided herein is the use of one or more compounds of the invention, or a pharmaceutically acceptable salt thereof, or a pharmaceutical composition comprising one or more compounds of the invention, for the preparation of a medicament for the treatment of the above-referenced diseases or conditions.

In another embodiment, provided herein the compounds of the invention, or a pharmaceutically acceptable salt thereof, or a pharmaceutical composition comprising one or more of the disclosed compounds are for use in treating the above-referenced diseases or conditions.

The details of one or more embodiments of the invention are set forth in the description below. Other features, objects, and advantages of the invention will be apparent from the description and from the claims. It should be understood that all embodiments/features of the invention (compounds, pharmaceutical compositions, methods of make/use, etc) described herein, including any specific features described in the examples and original claims, can combine with one another unless not applicable or explicitly disclaimed.

### DETAILED DESCRIPTION OF THE INVENTION

Exemplary compounds described herein include, but are not limited to, the following:
4-(2-((R)-4-(3,4-difluorobenzyl)-2-(2-isopropoxyphenyl)piperazin-1-yl)-7-azaspiro[3.5]nonan-7-yl)-N-((3-nitro-4-(((tetrahydro-2H-pyran-4-yl)methyl)amino)phenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-(3,4-difluorobenzyl)-2-(2-isopropoxyphenyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-(3,4-difluorobenzyl)-2-(2-isopropoxyphenyl)piperazin-1-yl)-7-azaspiro[3.5]nonan-7-yl)-5-fluoro-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-(3,4-difluorobenzyl)-2-(2-isopropoxyphenyl)piperazin-1-yl)-7-azaspiro[3.5]nonan-7-yl)-3,5-difluoro-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
6-(2-((R)-4-(3,4-difluorobenzyl)-2-(2-isopropoxyphenyl)piperazin-1-yl)-7-azaspiro[3.5]nonan-7-yl)-5-fluoro-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)nicotinamide,
6-(2-((R)-4-(3,4-difluorobenzyl)-2-(2-isopropoxyphenyl)piperazin-1-yl)-7-azaspiro[3.5]nonan-7-yl)-5-fluoro-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)nicotinamide,
6-(2-((R)-4-(3,4-difluorobenzyl)-2-(2-isopropoxyphenyl)piperazin-1-yl)-7-azaspiro[3.5]nonan-7-yl)-5-fluoro-N-(((R)-5-nitro-3-(tetrahydro-2H-pyran-4-yl)-3,4-dihydro-2H-benzo[b][1,4]oxazin-7-yl)sulfonyl)-4-((5aS,9aR)-6,7,9,9a-tetrahydro-1H-pyrano[3,4-b]pyrrolo[3′,2′:5,6]pyrido[3,2-e][1,4]oxazin-5(5aH)-yl)nicotinamide,
6-(2-((R)-4-(3,4-difluorobenzyl)-2-(2-isopropoxyphenyl)piperazin-1-yl)-7-azaspiro[3.5]nonan-7-yl)-5-fluoro-N-(((R)-5-nitro-3-(tetrahydro-2H-pyran-4-yl)-3,4-dihydro-2H-benzo[b][1,4]oxazin-7-yl)sulfonyl)-4-((5aS,9aR)-6,7,9,9a-tetrahydro-1H-pyrano[3,4-b]pyrrolo[3′,2′:5,6]pyrido[3,2-e][1,4]oxazin-5(5aH)-yl)nicotinamide,
6-(2-((R)-4-(3,4-difluorobenzyl)-2-(2-isopropoxyphenyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-4-((5aS,9aR)-6,7,9,9a-tetrahydro-1H-pyrano[3,4-b]pyrrolo[3′,2′:5,6]pyrido[3,2-e][1,4]oxazin-5(5aH)-yl)nicotinamide,
6-(2-((R)-4-(3,4-difluorobenzyl)-2-(2-isopropoxyphenyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)nicotinamide,
5-(2-((R)-4-(3,4-difluorobenzyl)-2-(2-isopropoxyphenyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-3-((5aS,9aR)-6,7,9,9a-tetrahydro-1H-pyrano[3,4-b]pyrrolo[3′,2′:5,6]pyrido[3,2-e][1,4]oxazin-5(5aH)-yl)picolinamide,
2-(2-((R)-4-(3,4-difluorobenzyl)-2-(2-isopropoxyphenyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-4-((5aS,9aR)-6,7,9,9a-tetrahydro-1H-pyrano[3,4-b]pyrrolo[3′,2′:5,6]pyrido[3,2-e][1,4]oxazin-5(5aH)-yl)pyrimidine-5-carboxamide,
5-(2-((R)-4-(3,4-difluorobenzyl)-2-(2-isopropoxyphenyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-3-((5aS,9aR)-6,7,9,9a-tetrahydro-1H-pyrano[3,4-b]pyrrolo[3′,2′:5,6]pyrido[3,2-e][1,4]oxazin-5(5aH)-yl)pyrazine-2-carboxamide,
4-(2-((R)-4-(3,4-difluorobenzyl)-2-(2-isopropoxyphenyl)piperazin-1-yl)-7-azaspiro[3.5]nonan-7-yl)-2-((5aS,9aR)-3-fluoro-6,7,9,9a-tetrahydro-1H-pyrano[3,4-b]pyrrolo[3′,2′:5,6]pyrido[3,2-e][1,4]oxazin-5(5aH)-yl)-N-(((R)-5-nitro-3-(tetrahydro-2H-pyran-4-yl)-3,4-dihydro-2H-benzo[b][1,4]oxazin-7-yl)sulfonyl)benzamide,
4-(2-((R)-4-((R)-1-(3,4-difluorophenyl)ethyl)-2-(2-isopropoxyphenyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-((S)-1-(3,4-difluorophenyl)ethyl)-2-(2-isopropoxyphenyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-(3,4-difluorobenzoyl)-2-(2-isopropoxyphenyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-((3,4-difluorophenyl)sulfonyl)-2-(2-isopropoxyphenyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-((R)-1-(3,4-difluorophenyl)propyl)-2-(2-isopropoxyphenyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-((S)-1-(3,4-difluorophenyl)propyl)-2-(2-isopropoxyphenyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-((R)-1-(3,4-difluorophenyl)-2-methylpropyl)-2-(2-isopropoxyphenyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-((S)-1-(3,4-difluorophenyl)-2-methylpropyl)-2-(2-isopropoxyphenyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-(3,4-difluorobenzyl)-2-(2-isopropoxyphenyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aR,9aS)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-(3,4-difluorobenzyl)-2-(2-isopropoxyphenyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aS)-5a,8,9,9a-tetrahydro-6H-pyrano[4,3-b]pyrrolo[3′,2′:5,6]pyrido[3,2-e][1,4]oxazin-5(1H)-yl)benzamide,
4-(2-((R)-4-(3,4-difluorobenzyl)-2-(2-isopropoxyphenyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aR,9aR)-5a,8,9,9a-tetrahydro-6H-pyrano[4,3-b]pyrrolo[3′,2′:5,6]pyrido[3,2-e][1,4]oxazin-5(1H)-yl)benzamide,
4-(2-((R)-4-(3,4-difluorobenzyl)-2-(2-isopropoxyphenyl)piperazin-1-yl)-7-azaspiro[3.5]nonan-7-yl)-2-((5aR,9aS)-9,9-dimethyl-6,7,9,9a-tetrahydro-1H-pyrano[3,4-b]pyrrolo[3′,2′:5,6]pyrido[3,2-e][1,4]oxazin-5(5aH)-yl)-N-(((R)-5-nitro-3-(tetrahydro-2H-pyran-4-yl)-3,4-dihydro-2H-benzo[b][1,4]oxazin-7-yl)sulfonyl)benzamide,
4-(2-((R)-4-(3,4-difluorobenzyl)-2-(2-isopropoxyphenyl)piperazin-1-yl)-7-azaspiro[3.5]nonan-7-yl)-2-((5aR,9aS)-7,7-dimethyl-6,7,9,9a-tetrahydro-1H-pyrano[3,4-b]pyrrolo[3′,2′:5,6]pyrido[3,2-e][1,4]oxazin-5(5aH)-yl)-N-(((R)-5-nitro-3-(tetrahydro-2H-pyran-4-yl)-3,4-dihydro-2H-benzo[b][1,4]oxazin-7-yl)sulfonyl)benzamide,
4-(2-((R)-4-(3,4-difluorobenzyl)-2-(2-isopropoxyphenyl)piperazin-1-yl)-7-azaspiro[3.5]nonan-7-yl)-N-(((R)-2,2-dimethyl-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-(3,4-difluorobenzyl)-2-(5-fluoro-2-isopropoxyphenyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-(3,4-difluorobenzyl)-2-(3-fluoro-2-isopropoxyphenyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-2-(3,5-difluoro-2-isopropoxyphenyl)-4-(3,4-difluorobenzyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-benzyl-2-(2-isopropoxyphenyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-(4-fluorobenzyl)-2-(2-isopropoxyphenyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-(4-chlorobenzyl)-2-(2-isopropoxyphenyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-2-(2-isopropoxyphenyl)-4-(4-methylbenzyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-2-(2-isopropoxyphenyl)-4-(4-methoxybenzyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-(3-chloro-4-fluorobenzyl)-2-(2-isopropoxyphenyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-(2-chloro-4-fluorobenzyl)-2-(2-isopropoxyphenyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-(4-chloro-3-fluorobenzyl)-2-(2-isopropoxyphenyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-(4-chloro-2-fluorobenzyl)-2-(2-isopropoxyphenyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-(3-fluoro-4-methylbenzyl)-2-(2-isopropoxyphenyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-(2-fluoro-4-methylbenzyl)-2-(2-isopropoxyphenyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-(3,4-difluoro-5-methylbenzyl)-2-(2-isopropoxyphenyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-(3-chloro-4-fluoro-5-methylbenzyl)-2-(2-isopropoxyphenyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-(4-chloro-3-fluoro-5-methylbenzyl)-2-(2-isopropoxyphenyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-(3,5-difluoro-4-methylbenzyl)-2-(2-isopropoxyphenyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-(3-chloro-5-fluoro-4-methylbenzyl)-2-(2-isopropoxyphenyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-(3,5-difluorobenzyl)-2-(2-isopropoxyphenyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-(3-chloro-5-fluorobenzyl)-2-(2-isopropoxyphenyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-(3-fluoro-5-methylbenzyl)-2-(2-isopropoxyphenyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-(3-chloro-5-methylbenzyl)-2-(2-isopropoxyphenyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-((1S,2S)-2-((R)-4-(3,4-difluorobenzyl)-2-(2-isopropoxyphenyl)piperazin-1-yl)-1-methyl-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-((1R,2S)-2-((R)-4-(3,4-difluorobenzyl)-2-(2-isopropoxyphenyl)piperazin-1-yl)-1-methyl-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-((2S,4R,6S)-2-((R)-4-(3,4-difluorobenzyl)-2-(2-isopropoxyphenyl)piperazin-1-yl)-6-methyl-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-((2R,4S,6S)-2-((R)-4-(3,4-difluorobenzyl)-2-(2-isopropoxyphenyl)piperazin-1-yl)-6-methyl-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-((2R,4R,6R)-2-((R)-4-(3,4-difluorobenzyl)-2-(2-isopropoxyphenyl)piperazin-1-yl)-6-methyl-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-((2S,4S,6R)-2-((R)-4-(3,4-difluorobenzyl)-2-(2-isopropoxyphenyl)piperazin-1-yl)-6-methyl-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-2-(2-isopropoxyphenyl)-4-((5-methoxy-6-((S)-3-methylmorpholino)pyridin-3-yl)methyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-((5,6-dimethoxypyridin-3-yl)methyl)-2-(2-isopropoxyphenyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-2-(2-isopropoxyphenyl)-4-((tetrahydro-2H-pyran-4-yl)methyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-2-(2-isopropoxyphenyl)-4-(tetrahydro-2H-pyran-4-yl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-2-(2-isopropoxyphenyl)-4-((R)-tetrahydro-2H-pyran-3-yl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-2-(2-isopropoxyphenyl)-4-((S)-tetrahydro-2H-pyran-3-yl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-((1r,4R)-4-hydroxy-4-methylcyclohexyl)-2-(2-isopropoxyphenyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-((1s,4S)-4-hydroxy-4-methylcyclohexyl)-2-(2-isopropoxyphenyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-2-(2-isopropoxyphenyl)-4-(3-oxaspiro[5.5]undecan-9-yl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-(4,4-dimethylcyclohexyl)-2-(2-isopropoxyphenyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-(bicyclo[1.1.1]pentan-1-ylmethyl)-2-(2-isopropoxyphenyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-((3-aminobicyclo[1.1.1]pentan-1-yl)methyl)-2-(2-isopropoxyphenyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-2-(2-isopropoxyphenyl)-4-((3-(trifluoromethyl)bicyclo[1.1.1]pentan-1-yl)methyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-(bicyclo[2.2.2]octan-1-ylmethyl)-2-(2-isopropoxyphenyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(((R)-3-(2-isopropoxyphenyl)-4-(7-(4-((((R)-5-nitro-3-(tetrahydro-2H-pyran-4-yl)-3,4-dihydro-2H-benzo[b][1,4]oxazin-7-yl)sulfonyl)carbamoyl)-3-((5aS,9aR)-6,7,9,9a-tetrahydro-1H-pyrano[3,4-b]pyrrolo[3′,2′:5,6]pyrido[3,2-e][1,4]oxazin-5(5aH)-yl)phenyl)-7-azaspiro[3.5]nonan-2-yl)piperazin-1-yl)methyl)bicyclo[2.2.2]octane-1-carboxamide,
4-(2-((R)-2-(2-isopropoxyphenyl)-4-((4-(trifluoromethyl)bicyclo[2.2.2]octan-1-yl)methyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-2-(2-isopropoxyphenyl)-4-((S)-8-methoxy-7-((S)-3-methylmorpholino)-3,4-dihydro-1H-pyrano[4,3-c]pyridin-4-yl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-2-(2-isopropoxyphenyl)-4-((R)-8-methoxy-7-((S)-3-methylmorpholino)-3,4-dihydro-1H-pyrano[4,3-c]pyridin-4-yl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-2-(2-isopropoxyphenyl)-4-((S)-3-methoxy-2-((S)-3-methylmorpholino)-5,8-dihydro-6H-pyrano[3,4-b]pyridin-5-yl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-2-(2-isopropoxyphenyl)-4-((R)-3-methoxy-2-((S)-3-methylmorpholino)-5,8-dihydro-6H-pyrano[3,4-b]pyridin-5-yl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-((S)-6-fluoro-7-((S)-3-methyl morpholino)-3,4-dihydro-2H-pyrano[2,3-b]pyridin-4-yl)-2-(2-isopropoxyphenyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-((R)-6-fluoro-7-((S)-3-methyl morpholino)-3,4-dihydro-2H-pyrano[2,3-b]pyridin-4-yl)-2-(2-isopropoxyphenyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-((S)-7,8-dimethoxy-3,4-dihydro-1H-pyrano[4,3-c]pyridin-4-yl)-2-(2-isopropoxyphenyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-((R)-7,8-dimethoxy-3,4-dihydro-1H-pyrano[4,3-c]pyridin-4-yl)-2-(2-isopropoxyphenyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-((S)-2,3-dimethoxy-5,8-dihydro-6H-pyrano[3,4-b]pyridin-5-yl)-2-(2-isopropoxyphenyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-((R)-2,3-dimethoxy-5,8-dihydro-6H-pyrano[3,4-b]pyridin-5-yl)-2-(2-isopropoxyphenyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-((S)-10,10-dimethyl-6-((S)-3-methylmorpholino)-1,8,9,10-tetrahydro-2H,4H-dipyrano[3,4-b:3′,2′-d]pyridin-1-yl)-2-(2-isopropoxyphenyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-((R)-10,10-dimethyl-6-((S)-3-methylmorpholino)-1,8,9,10-tetrahydro-2H,4H-dipyrano[3,4-b:3′,2′-d]pyridin-1-yl)-2-(2-isopropoxyphenyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-2-(2-isopropoxyphenyl)-4-((S)-3-methoxy-8,8-dimethyl-2-((S)-3-methylmorpholino)-5,8-dihydro-6H-pyrano[3,4-b]pyridin-5-yl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-2-(2-isopropoxyphenyl)-4-((R)-3-methoxy-8,8-dimethyl-2-((S)-3-methylmorpholino)-5,8-dihydro-6H-pyrano[3,4-b]pyridin-5-yl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-2-(2-isopropoxyphenyl)-4-((S)-4,4,10,10-tetramethyl-6-((S)-3-methylmorpholino)-1,8,9,10-tetrahydro-2H,4H-dipyrano[3,4-b:3′,2′-d]pyridin-1-yl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-2-(2-isopropoxyphenyl)-4-((S)-4,4,10,10-tetramethyl-1,8,9,10-tetrahydro-2H,4H-dipyrano[3,4-b:3′,2′-d]pyridin-1-yl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-2-(2-isopropoxyphenyl)-4-((S)-4,4,6,10,10-pentamethyl-1,8,9,10-tetrahydro-2H,4H-dipyrano[3,4-b:3′,2′-d]pyridin-1-yl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-((S)-6-cyclopropyl-4,4,10,10-tetramethyl-1,8,9,10-tetrahydro-2H,4H-dipyrano[3,4-b:3′,2′-d]pyridin-1-yl)-2-(2-isopropoxyphenyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-2-(2-isopropoxyphenyl)-4-((S)-6-isopropyl-4,4,10,10-tetramethyl-1,8,9,10-tetrahydro-2H,4H-dipyrano[3,4-b:3′,2′-d]pyridin-1-yl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-2-(2-isopropoxyphenyl)-4-((S)-4,4,10,10-tetramethyl-6-(methylamino)-1,8,9,10-tetrahydro-2H,4H-dipyrano[3,4-b:3′,2′-d]pyridin-1-yl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-2-(2-isopropoxyphenyl)-4-((S)-6-methoxy-4,4,10,10-tetramethyl-1,8,9,10-tetrahydro-2H,4H-dipyrano[3,4-b:3′,2′-d]pyridin-1-yl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-((S)-6-cyano-4,4,10,10-tetramethyl-1,8,9,10-tetrahydro-2H,4H-dipyrano[3,4-b:3′,2′-d]pyridin-1-yl)-2-(2-isopropoxyphenyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-2-(2-isopropoxyphenyl)-4-((S)-4,4,10,10-tetramethyl-6-(trifluoromethyl)-1,8,9,10-tetrahydro-2H,4H-dipyrano[3,4-b:3′,2′-d]pyridin-1-yl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-2-(2-isopropoxyphenyl)-4-((S)-4,4,10,10-tetramethyl-6-(methylsulfonyl)-1,8,9,10-tetrahydro-2H,4H-dipyrano[3,4-b:3′,2′-d]pyridin-1-yl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-2-(2-isopropoxyphenyl)-4-((S)-3,4,8,9-tetrahydro-2H,6H-pyrano[4′,3′:5,6]pyrido[3,2-b][1,4]oxazin-9-yl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-2-(2-isopropoxyphenyl)-4-((S)-2,3,8,9-tetrahydro-6H-[1,4]dioxino[2,3-b]pyrano[4,3-e]pyridin-9-yl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-2-(2-isopropoxyphenyl)-4-((S)-3,4,8,9-tetrahydro-2H,6H-dipyrano[3,2-b:4′,3′-e]pyridin-9-yl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-((4aS,8S)-11,11-dimethyl-1,2,4a,5,8,11-hexahydro-4H,9H-[1,4]oxazino[4,3-d]pyrano[4′,3′:5,6]pyrido[3,2-b][1,4]oxazin-8-yl)-2-(2-isopropoxyphenyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-2-(2-isopropoxyphenyl)-4-((S)-3,4,7,10-tetrahydro-2H,8H-pyrano[3′,4′:4,5]pyrido[3,2-b][1,4]oxazin-7-yl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-2-(2-isopropoxyphenyl)-4-((3S,7S)-3-methyl-3,4,7,10-tetrahydro-2H,8H-pyrano[3′,4′:4,5]pyrido[3,2-b][1,4]oxazin-7-yl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-2-(2-isopropoxyphenyl)-4-((3R,7S)-3-methyl-3,4,7,10-tetrahydro-2H,8H-pyrano[3′,4′:4,5]pyrido[3,2-b][1,4]oxazin-7-yl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-((3R,7S)-4-((R)-1-hydroxypropan-2-yl)-3-methyl-3,4,7,10-tetrahydro-2H,8H-pyrano[3′,4′:4,5]pyrido[3,2-b][1,4]oxazin-7-yl)-2-(2-isopropoxyphenyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-2-(2-isopropoxyphenyl)-4-((3S,7S)-3-methyl-4-(tetrahydro-2H-pyran-4-yl)-3,4,7,10-tetrahydro-2H,8H-pyrano[3′,4′:4,5]pyrido[3,2-b][1,4]oxazin-7-yl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-((S)-6,8-difluorochroman-3-yl)-2-(2-isopropoxyphenyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-((R)-6,8-difluorochroman-3-yl)-2-(2-isopropoxyphenyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-((S)-6,7-difluorochroman-4-yl)-2-(2-isopropoxyphenyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-((R)-6,7-difluorochroman-4-yl)-2-(2-isopropoxyphenyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-((R)-7,8-difluorochroman-4-yl)-2-(2-isopropoxyphenyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-((S)-7,8-difluorochroman-4-yl)-2-(2-isopropoxyphenyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-((S)-6,8-difluorochroman-4-yl)-2-(2-isopropoxyphenyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-((R)-6,8-difluorochroman-4-yl)-2-(2-isopropoxyphenyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-((R)-6,7-difluoroisochroman-4-yl)-2-(2-isopropoxyphenyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-((S)-6,7-difluoroisochroman-4-yl)-2-(2-isopropoxyphenyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-((S)-7,8-difluoroisochroman-4-yl)-2-(2-isopropoxyphenyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-((R)-7,8-difluoroisochroman-4-yl)-2-(2-isopropoxyphenyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-((R)-6,8-difluoroisochroman-4-yl)-2-(2-isopropoxyphenyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-((S)-6,8-difluoroisochroman-4-yl)-2-(2-isopropoxyphenyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-((S)-3,4-dihydro-2H-pyrano[2,3-b]pyridin-3-yl)-2-(2-isopropoxyphenyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-((R)-3,4-dihydro-2H-pyrano[2,3-b]pyridin-3-yl)-2-(2-isopropoxyphenyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-((S)-3,4-dihydro-2H-pyrano[2,3-c]pyridin-3-yl)-2-(2-isopropoxyphenyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-((R)-3,4-dihydro-2H-pyrano[2,3-c]pyridin-3-yl)-2-(2-isopropoxyphenyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-((S)-3,4-dihydro-2H-pyrano[3,2-c]pyridin-3-yl)-2-(2-isopropoxyphenyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-((R)-3,4-dihydro-2H-pyrano[3,2-c]pyridin-3-yl)-2-(2-isopropoxyphenyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-((S)-3,4-dihydro-2H-pyrano[3,2-b]pyridin-3-yl)-2-(2-isopropoxyphenyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-((R)-3,4-dihydro-2H-pyrano[3,2-b]pyridin-3-yl)-2-(2-isopropoxyphenyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-((S)-6,7-dihydro-5H-pyrano[2,3-d]pyrimidin-6-yl)-2-(2-isopropoxyphenyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-((R)-6,7-dihydro-5H-pyrano[2,3-d]pyrimidin-6-yl)-2-(2-isopropoxyphenyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-((S)-7,8-dihydro-6H-pyrano[3,2-d]pyrimidin-7-yl)-2-(2-isopropoxyphenyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-((R)-7,8-dihydro-6H-pyrano[3,2-d]pyrimidin-7-yl)-2-(2-isopropoxyphenyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-((S)-7,8-dihydro-6H-pyrano[2,3-b]pyrazin-7-yl)-2-(2-isopropoxyphenyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-((R)-7,8-dihydro-6H-pyrano[2,3-b]pyrazin-7-yl)-2-(2-isopropoxyphenyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-((S)-5,8-dihydro-6H-pyrano[3,4-b]pyridin-5-yl)-2-(2-isopropoxyphenyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-((R)-5,8-dihydro-6H-pyrano[3,4-b]pyridin-5-yl)-2-(2-isopropoxyphenyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-((S)-3,4-dihydro-1H-pyrano[3,4-c]pyridin-4-yl)-2-(2-isopropoxyphenyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-((R)-3,4-dihydro-1H-pyrano[3,4-c]pyridin-4-yl)-2-(2-isopropoxyphenyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-((S)-3,4-dihydro-1H-pyrano[4,3-c]pyridin-4-yl)-2-(2-isopropoxyphenyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-((R)-3,4-dihydro-1H-pyrano[4,3-c]pyridin-4-yl)-2-(2-isopropoxyphenyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-((S)-7,8-dihydro-5H-pyrano[4,3-b]pyridin-8-yl)-2-(2-isopropoxyphenyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-((R)-7,8-dihydro-5H-pyrano[4,3-b]pyridin-8-yl)-2-(2-isopropoxyphenyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-((S)-5,8-dihydro-6H-pyrano[3,4-d]pyrimidin-5-yl)-2-(2-isopropoxyphenyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-((R)-5,8-dihydro-6H-pyrano[3,4-d]pyrimidin-5-yl)-2-(2-isopropoxyphenyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-((S)-7,8-dihydro-5H-pyrano[4,3-d]pyrimidin-8-yl)-2-(2-isopropoxyphenyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-((R)-7,8-dihydro-5H-pyrano[4,3-d]pyrimidin-8-yl)-2-(2-isopropoxyphenyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-((S)-7,8-dihydro-5H-pyrano[3,4-b]pyrazin-8-yl)-2-(2-isopropoxyphenyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-((R)-7,8-dihydro-5H-pyrano[3,4-b]pyrazin-8-yl)-2-(2-isopropoxyphenyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-((R)-3,4-dihydro-2H-pyrano[3,2-c]pyridin-4-yl)-2-(2-isopropoxyphenyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-((S)-3,4-dihydro-2H-pyrano[3,2-c]pyridin-4-yl)-2-(2-isopropoxyphenyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-((R)-3,4-dihydro-2H-pyrano[2,3-c]pyridin-4-yl)-2-(2-isopropoxyphenyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-((S)-3,4-dihydro-2H-pyrano[2,3-c]pyridin-4-yl)-2-(2-isopropoxyphenyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-((R)-3,4-dihydro-2H-pyrano[2,3-b]pyridin-4-yl)-2-(2-isopropoxyphenyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-((S)-3,4-dihydro-2H-pyrano[2,3-b]pyridin-4-yl)-2-(2-isopropoxyphenyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-((R)-3,4-dihydro-2H-pyrano[3,2-b]pyridin-4-yl)-2-(2-isopropoxyphenyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-((S)-3,4-dihydro-2H-pyrano[3,2-b]pyridin-4-yl)-2-(2-isopropoxyphenyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-((R)-6,7-dihydro-5H-pyrano[2,3-d]pyrimidin-5-yl)-2-(2-isopropoxyphenyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-((S)-6,7-dihydro-5H-pyrano[2,3-d]pyrimidin-5-yl)-2-(2-isopropoxyphenyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-((R)-7,8-dihydro-6H-pyrano[3,2-d]pyrimidin-8-yl)-2-(2-isopropoxyphenyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-((S)-7,8-dihydro-6H-pyrano[3,2-d]pyrimidin-8-yl)-2-(2-isopropoxyphenyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-((R)-7,8-dihydro-6H-pyrano[2,3-b]pyrazin-8-yl)-2-(2-isopropoxyphenyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-((S)-7,8-dihydro-6H-pyrano[2,3-b]pyrazin-8-yl)-2-(2-isopropoxyphenyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-((R)-isochroman-4-yl)-2-(2-isopropoxyphenyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-((S)-isochroman-4-yl)-2-(2-isopropoxyphenyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-((R)-6-chloro-8-fluoroisochroman-4-yl)-2-(2-isopropoxyphenyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-((S)-6-chloro-8-fluoroisochroman-4-yl)-2-(2-isopropoxyphenyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-((R)-8-fluoro-6-methylisochroman-4-yl)-2-(2-isopropoxyphenyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-((S)-8-fluoro-6-methylisochroman-4-yl)-2-(2-isopropoxyphenyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-((R)-6-chloro-8-methylisochroman-4-yl)-2-(2-isopropoxyphenyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-((S)-6-chloro-8-methylisochroman-4-yl)-2-(2-isopropoxyphenyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-((R)-8-chloro-6-methylisochroman-4-yl)-2-(2-isopropoxyphenyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-((S)-8-chloro-6-methylisochroman-4-yl)-2-(2-isopropoxyphenyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-((S)-chroman-4-yl)-2-(2-isopropoxyphenyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-((R)-chroman-4-yl)-2-(2-isopropoxyphenyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-((S)-6-chloro-8-fluorochroman-4-yl)-2-(2-isopropoxyphenyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-((R)-6-chloro-8-fluorochroman-4-yl)-2-(2-isopropoxyphenyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-((S)-8-fluoro-6-methylchroman-4-yl)-2-(2-isopropoxyphenyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-((R)-8-fluoro-6-methylchroman-4-yl)-2-(2-isopropoxyphenyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-((S)-6-chloro-8-methylchroman-4-yl)-2-(2-isopropoxyphenyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-((R)-6-chloro-8-methylchroman-4-yl)-2-(2-isopropoxyphenyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-((S)-8-chloro-6-methylchroman-4-yl)-2-(2-isopropoxyphenyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-((R)-8-chloro-6-methylchroman-4-yl)-2-(2-isopropoxyphenyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-2-(2-isopropoxyphenyl)-4-phenylpiperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-(2,4-difluoro-6-methylphenyl)-2-(2-isopropoxyphenyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-(2-chloro-4-fluoro-6-methylphenyl)-2-(2-isopropoxyphenyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-(4-chloro-2,6-difluorophenyl)-2-(2-isopropoxyphenyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-(4-chloro-2-fluoro-6-methylphenyl)-2-(2-isopropoxyphenyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-(2,6-difluoro-4-methylphenyl)-2-(2-isopropoxyphenyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-(2-chloro-6-fluoro-4-methylphenyl)-2-(2-isopropoxyphenyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-(1,3-dioxo-2-(tetrahydro-2H-pyran-4-yl)isoindolin-4-yl)-2-(2-isopropoxyphenyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((2R)-2-(2-isopropoxyphenyl)-4-(2-(1-methyl-2,6-dioxopiperidin-3-yl)-1,3-dioxoisoindolin-4-yl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-(5,7-difluorochroman-6-yl)-2-(2-isopropoxyphenyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-(5,7-difluoroisochroman-6-yl)-2-(2-isopropoxyphenyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-(6,8-difluoroisochroman-7-yl)-2-(2-isopropoxyphenyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-(6,8-difluorochroman-7-yl)-2-(2-isopropoxyphenyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-(6,8-difluorochroman-5-yl)-2-(2-isopropoxyphenyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-(6,8-difluoroisochroman-5-yl)-2-(2-isopropoxyphenyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-(5,7-difluoroisochroman-8-yl)-2-(2-isopropoxyphenyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-(6,7-difluorochroman-8-yl)-2-(2-isopropoxyphenyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-2-(2-isopropoxyphenyl)-4-(pyridin-4-yl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-2-(2-isopropoxyphenyl)-4-(pyridin-3-yl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-2-(2-isopropoxyphenyl)-4-(pyridin-2-yl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-2-(2-isopropoxyphenyl)-4-(pyrazin-2-yl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-2-(2-isopropoxyphenyl)-4-(pyrimidin-2-yl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-2-(2-isopropoxyphenyl)-4-(pyrimidin-4-yl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-2-(2-isopropoxyphenyl)-4-(pyrimidin-5-yl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-(6,7-dihydro-5H-pyrano[2,3-d]pyrimidin-2-yl)-2-(2-isopropoxyphenyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-(5,8-dihydro-6H-pyrano[3,4-d]pyrimidin-2-yl)-2-(2-isopropoxyphenyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-(7,8-dihydro-5H-pyrano[4,3-d]pyrimidin-2-yl)-2-(2-isopropoxyphenyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-(7,8-dihydro-6H-pyrano[3,2-d]pyrimidin-2-yl)-2-(2-isopropoxyphenyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-(7,8-dihydro-6H-pyrano[2,3-b]pyrazin-2-yl)-2-(2-isopropoxyphenyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-(7,8-dihydro-5H-pyrano[3,4-b]pyrazin-2-yl)-2-(2-isopropoxyphenyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-(7,8-dihydro-5H-pyrano[3,4-b]pyrazin-3-yl)-2-(2-isopropoxyphenyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-(7,8-dihydro-6H-pyrano[2,3-b]pyrazin-3-yl)-2-(2-isopropoxyphenyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-(6,7-dihydro-5H-pyrano[2,3-d]pyrimidin-4-yl)-2-(2-isopropoxyphenyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-(5,8-dihydro-6H-pyrano[3,4-d]pyrimidin-4-yl)-2-(2-isopropoxyphenyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-(7,8-dihydro-5H-pyrano[4,3-d]pyrimidin-4-yl)-2-(2-isopropoxyphenyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-(7,8-dihydro-6H-pyrano[3,2-d]pyrimidin-4-yl)-2-(2-isopropoxyphenyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-(3,4-dihydro-2H-pyrano[3,2-c]pyridin-5-yl)-2-(2-isopropoxyphenyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-(3,4-dihydro-1H-pyrano[4,3-c]pyridin-5-yl)-2-(2-isopropoxyphenyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-(3,4-dihydro-1H-pyrano[3,4-c]pyridin-8-yl)-2-(2-isopropoxyphenyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-(3,4-dihydro-2H-pyrano[2,3-c]pyridin-8-yl)-2-(2-isopropoxyphenyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-(3,4-dihydro-2H-pyrano[2,3-c]pyridin-6-yl)-2-(2-isopropoxyphenyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-(3,4-dihydro-1H-pyrano[3,4-c]pyridin-6-yl)-2-(2-isopropoxyphenyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-(3,4-dihydro-1H-pyrano[4,3-c]pyridin-7-yl)-2-(2-isopropoxyphenyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-(3,4-dihydro-2H-pyrano[3,2-c]pyridin-7-yl)-2-(2-isopropoxyphenyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-(3,4-dihydro-2H-pyrano[2,3-b]pyridin-7-yl)-2-(2-isopropoxyphenyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-(5,8-dihydro-6H-pyrano[3,4-b]pyridin-2-yl)-2-(2-isopropoxyphenyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-(7,8-dihydro-5H-pyrano[4,3-b]pyridin-2-yl)-2-(2-isopropoxyphenyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-(3,4-dihydro-2H-pyrano[3,2-b]pyridin-6-yl)-2-(2-isopropoxyphenyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-(3,4-dihydro-2H-pyrano[2,3-b]pyridin-6-yl)-2-(2-isopropoxyphenyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-(5,8-dihydro-6H-pyrano[3,4-b]pyridin-3-yl)-2-(2-isopropoxyphenyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-(7,8-dihydro-5H-pyrano[4,3-b]pyridin-3-yl)-2-(2-isopropoxyphenyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-(3,4-dihydro-2H-pyrano[3,2-b]pyridin-7-yl)-2-(2-isopropoxyphenyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-(3,4-dihydro-2H-pyrano[2,3-c]pyridin-5-yl)-2-(2-isopropoxyphenyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-(3,4-dihydro-1H-pyrano[3,4-c]pyridin-5-yl)-2-(2-isopropoxyphenyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-(3,4-dihydro-1H-pyrano[4,3-c]pyridin-8-yl)-2-(2-isopropoxyphenyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-(3,4-dihydro-2H-pyrano[3,2-c]pyridin-8-yl)-2-(2-isopropoxyphenyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-(3,4-dihydro-2H-pyrano[2,3-b]pyridin-5-yl)-2-(2-isopropoxyphenyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-(5,8-dihydro-6H-pyrano[3,4-b]pyridin-4-yl)-2-(2-isopropoxyphenyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-(7,8-dihydro-5H-pyrano[4,3-b]pyridin-4-yl)-2-(2-isopropoxyphenyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-(3,4-dihydro-2H-pyrano[3,2-b]pyridin-8-yl)-2-(2-isopropoxyphenyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-((1s,3S)-3-(3,4-difluorophenyl)cyclobutyl)-2-(2-isopropoxyphenyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-((1r,3R)-3-(3,4-difluorophenyl)cyclobutyl)-2-(2-isopropoxyphenyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-((1R,2S)-2-(3,4-difluorophenyl)cyclobutyl)-2-(2-isopropoxyphenyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-((1S,2R)-2-(3,4-difluorophenyl)cyclobutyl)-2-(2-isopropoxyphenyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-((1S,2S)-2-(3,4-difluorophenyl)cyclobutyl)-2-(2-isopropoxyphenyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-((1R,2R)-2-(3,4-difluorophenyl)cyclobutyl)-2-(2-isopropoxyphenyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-2-(2-isopropoxyphenyl)-4-((1R,3S)-3-(3-methoxy-4-((S)-3-methylmorpholino)phenyl)cyclobutyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-2-(2-isopropoxyphenyl)-4-((1S,3R)-3-(3-methoxy-4-((S)-3-methylmorpholino)phenyl)cyclobutyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-2-(2-isopropoxyphenyl)-4-((1R,2S)-2-(5-methoxy-6-((S)-3-methylmorpholino)pyridin-3-yl)cyclobutyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-2-(2-isopropoxyphenyl)-4-((1S,2R)-2-(5-methoxy-6-((S)-3-methylmorpholino)pyridin-3-yl)cyclobutyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-2-(2-isopropoxyphenyl)-4-((1S,2S)-2-(5-methoxy-6-((S)-3-methylmorpholino)pyridin-3-yl)cyclobutyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-2-(2-isopropoxyphenyl)-4-((1R,2R)-2-(5-methoxy-6-((S)-3-methylmorpholino)pyridin-3-yl)cyclobutyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-(3,4-difluorobenzyl)-2-(o-tolyl)piperazin-1-yl)-7-azaspiro[3.5]nonan-7-yl)-N-((4-((((1r,4r)-4-hydroxy-4-methylcyclohexyl)methyl)amino)-3-nitrophenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-(3,4-difluorobenzyl)-2-(o-tolyl)piperazin-1-yl)-7-azaspiro[3.5]nonan-7-yl)-N-((3-nitro-4-(((tetrahydro-2H-pyran-4-yl)methyl)amino)phenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-(3,4-difluorobenzyl)-2-(o-tolyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-(3,4-difluorobenzyl)-2-(o-tolyl)piperazin-1-yl)-7-azaspiro[3.5]nonan-7-yl)-N—(((S)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-(3,4-difluorobenzoyl)-2-(o-tolyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-((3,4-difluorophenyl)sulfonyl)-2-(o-tolyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-((R)-1-(3,4-difluorophenyl)propyl)-2-(o-tolyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-((S)-1-(3,4-difluorophenyl)propyl)-2-(o-tolyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-((R)-1-(3,4-difluorophenyl)-2-methylpropyl)-2-(o-tolyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-((S)-1-(3,4-difluorophenyl)-2-methylpropyl)-2-(o-tolyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-((((1r,4r)-4-hydroxy-4-methylcyclohexyl)methyl)amino)-3-nitrophenyl)sulfonyl)-4-(2-((R)-4-((5-methoxy-6-morpholinopyridin-3-yl)methyl)-2-(o-tolyl)piperazin-1-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-((5,6-dimethoxypyridin-3-yl)methyl)-2-(o-tolyl)piperazin-1-yl)-7-azaspiro[3.5]nonan-7-yl)-N-((4-((((1r,4r)-4-hydroxy-4-methylcyclohexyl)methyl)amino)-3-nitrophenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-((5-methoxy-6-morpholinopyridin-3-yl)methyl)-2-(o-tolyl)piperazin-1-yl)-7-azaspiro[3.5]nonan-7-yl)-N-((3-nitro-4-(((tetrahydro-2H-pyran-4-yl)methyl)amino)phenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-((5-methoxy-6-morpholinopyridin-3-yl)methyl)-2-(o-tolyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-((R)-1-(5-methoxy-6-((S)-3-methylmorpholino)pyridin-3-yl)ethyl)-2-(o-tolyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-((5-methoxy-6-morpholinopyridin-3-yl)methyl)-2-(o-tolyl)piperazin-1-yl)-7-azaspiro[3.5]nonan-7-yl)-N-((3-nitro-4-(((tetrahydro-2H-pyran-4-yl)methyl)amino)phenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N-((4-((((1r,4r)-4-hydroxy-4-methylcyclohexyl)methyl)amino)-3-nitrophenyl)sulfonyl)-4-(2-((R)-4-((5-methoxy-6-morpholinopyridin-3-yl)methyl)-2-(o-tolyl)piperazin-1-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-((5-methoxy-6-morpholinopyridin-3-yl)methyl)-2-(o-tolyl)piperazin-1-yl)-7-azaspiro[3.5]nonan-7-yl)-N—(((S)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-((5-methoxy-6-morpholinopyridin-3-yl)methyl)-2-(o-tolyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-((5-methoxy-6-((S)-3-methylmorpholino)pyridin-3-yl)methyl)-2-(o-tolyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-((5-methoxy-6-((R)-3-methylmorpholino)pyridin-3-yl)methyl)-2-(o-tolyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-((5-fluoro-6-((S)-3-methylmorpholino)pyridin-3-yl)methyl)-2-(o-tolyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-((5-methyl-6-((S)-3-methylmorpholino)pyridin-3-yl)methyl)-2-(o-tolyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-((6-((S)-3-methylmorpholino)-5-(trifluoromethyl)pyridin-3-yl)methyl)-2-(o-tolyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-((S)-1-(5-methoxy-6-((S)-3-methylmorpholino)pyridin-3-yl)ethyl)-2-(o-tolyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-((R)-1-(5-methoxy-6-((S)-3-methylmorpholino)pyridin-3-yl)ethyl)-2-(o-tolyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-(5-methoxy-6-((S)-3-methylmorpholino)nicotinoyl)-2-(o-tolyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-((5-methoxy-6-((S)-3-methylmorpholino)pyridin-3-yl)sulfonyl)-2-(o-tolyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-((R)-1-(5-methoxy-6-((S)-3-methylmorpholino)pyridin-3-yl)propyl)-2-(o-tolyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-((S)-1-(5-methoxy-6-((S)-3-methylmorpholino)pyridin-3-yl)propyl)-2-(o-tolyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-((R)-1-(5-methoxy-6-((S)-3-methylmorpholino)pyridin-3-yl)-2-methylpropyl)-2-(o-tolyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-((S)-1-(5-methoxy-6-((S)-3-methylmorpholino)pyridin-3-yl)-2-methylpropyl)-2-(o-tolyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-((5-isopropoxy-6-((S)-3-methylmorpholino)pyridin-3-yl)methyl)-2-(o-tolyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-((2-methyl-7-((S)-3-methylmorpholino)furo[2,3-c]pyridin-4-yl)methyl)-2-(o-tolyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-((4,4-dimethyl-8-morpholino-3,4-dihydro-2H-pyrano[2,3-c]pyridin-5-yl)methyl)-2-(o-tolyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-((4,4-dimethyl-8-((S)-3-methyl morpholino)-3,4-dihydro-2H-pyrano[2,3-c]pyridin-5-yl)methyl)-2-(o-tolyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-((S)-1-(4,4-dimethyl-8-((S)-3-methylmorpholino)-3,4-dihydro-2H-pyrano[2,3-c]pyridin-5-yl)ethyl)-2-(o-tolyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-((R)-1-(4,4-dimethyl-8-((S)-3-methylmorpholino)-3,4-dihydro-2H-pyrano[2,3-c]pyridin-5-yl)ethyl)-2-(o-tolyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-((S)-1-(4,4-dimethyl-3,4-dihydro-2H-pyrano[2,3-c]pyridin-5-yl)ethyl)-2-(o-tolyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N—(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)-4-(2-((R)-2-(o-tolyl)-4-((S)-1-(4,4,8-trimethyl-3,4-dihydro-2H-pyrano[2,3-c]pyridin-5-yl)ethyl)piperazin-1-yl)-7-azaspiro[3.5]nonan-7-yl)benzamide,
4-(2-((R)-4-((S)-1-(8-isopropyl-4,4-dimethyl-3,4-dihydro-2H-pyrano[2,3-c]pyridin-5-yl)ethyl)-2-(o-tolyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-((S)-1-(8-cyclopropyl-4,4-dimethyl-3,4-dihydro-2H-pyrano[2,3-c]pyridin-5-yl)ethyl)-2-(o-tolyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-((S)-1-(8-cyclobutyl-4,4-dimethyl-3,4-dihydro-2H-pyrano[2,3-c]pyridin-5-yl)ethyl)-2-(o-tolyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-((S)-1-(8-(tert-butyl)-4,4-dimethyl-3,4-dihydro-2H-pyrano[2,3-c]pyridin-5-yl)ethyl)-2-(o-tolyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-((S)-1-(4,4-dimethyl-8-(trifluoromethyl)-3,4-dihydro-2H-pyrano[2,3-c]pyridin-5-yl)ethyl)-2-(o-tolyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-((S)-1-(4,4-dimethyl-8-(1,1,1-trifluoro-2-methylpropan-2-yl)-3,4-dihydro-2H-pyrano[2,3-c]pyridin-5-yl)ethyl)-2-(o-tolyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-((S)-1-(4,4-dimethyl-8-(methylsulfonyl)-3,4-dihydro-2H-pyrano[2,3-c]pyridin-5-yl)ethyl)-2-(o-tolyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-((S)-1-(4,4-dimethyl-8-((trifluoromethyl)sulfonyl)-3,4-dihydro-2H-pyrano[2,3-c]pyridin-5-yl)ethyl)-2-(o-tolyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-((S)-1-(8-methoxy-4,4-dimethyl-3,4-dihydro-2H-pyrano[2,3-c]pyridin-5-yl)ethyl)-2-(o-tolyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N—(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)-4-(2-((R)-2-(o-tolyl)-4-((S)-1-(4,4,7-trimethyl-8-oxo-3,4,7,8-tetrahydro-2H-pyrano[2,3-c]pyridin-5-yl)ethyl)piperazin-1-yl)-7-azaspiro[3.5]nonan-7-yl)benzamide,
4-(2-((R)-4-((5,6-dimethoxypyridin-3-yl)methyl)-2-(o-tolyl)piperazin-1-yl)-7-azaspiro[3.5]nonan-7-yl)-N-((3-nitro-4-(((tetrahydro-2H-pyran-4-yl)methyl)amino)phenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-((5,6-dimethoxypyridin-3-yl)methyl)-2-(o-tolyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-((5,6-dimethoxypyridin-3-yl)methyl)-2-(o-tolyl)piperazin-1-yl)-7-azaspiro[3.5]nonan-7-yl)-N—(((S)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-((5,6-dimethoxypyridin-3-yl)methyl)-2-(o-tolyl)piperazin-1-yl)-7-azaspiro[3.5]nonan-7-yl)-N-((4-((((1r,4r)-4-hydroxy-4-methylcyclohexyl)methyl)amino)-3-nitrophenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
5-fluoro-4-(2-((R)-4-((5-methoxy-6-((S)-3-methyl morpholino)pyridin-3-yl)methyl)-2-(o-tolyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
3,5-difluoro-4-(2-((R)-4-((5-methoxy-6-morpholinopyridin-3-yl)methyl)-2-(o-tolyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
5-fluoro-6-(2-((R)-4-((5-methoxy-6-morpholinopyridin-3-yl)methyl)-2-(o-tolyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-4-((5aS,9aR)-6,7,9,9a-tetrahydro-1H-pyrano[3,4-b]pyrrolo[3′,2′:5,6]pyrido[3,2-e][1,4]oxazin-5(5aH)-yl)nicotinamide,
5-fluoro-6-(2-((R)-4-((5-methoxy-6-morpholinopyridin-3-yl)methyl)-2-(o-tolyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)nicotinamide,
4-((1R,2R)-2-((R)-4-((5-methoxy-6-((S)-3-methylmorpholino)pyridin-3-yl)methyl)-2-(o-tolyl)piperazin-1-yl)-1-methyl-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-((1S,2R)-2-((R)-4-((5-methoxy-6-((S)-3-methylmorpholino)pyridin-3-yl)methyl)-2-(o-tolyl)piperazin-1-yl)-1-methyl-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
2-((5aS,9aR)-3-fluoro-6,7,9,9a-tetrahydro-1H-pyrano[3,4-b]pyrrolo[3′,2′:5,6]pyrido[3,2-e][1,4]oxazin-5(5aH)-yl)-4-(2-((R)-4-((5-methoxy-6-((S)-3-methyl morpholino)pyridin-3-yl)methyl)-2-(o-tolyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)benzamide,
4-(2-((R)-4-((5-methoxy-6-((S)-3-methylmorpholino)pyridin-3-yl)methyl)-2-(o-tolyl)piperazin-1-yl)-7-azaspiro[3.5]nonan-7-yl)-N-(((2S,3R)-2-methyl-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-((5-methoxy-6-((S)-3-methylmorpholino)pyridin-3-yl)methyl)-2-(o-tolyl)piperazin-1-yl)-7-azaspiro[3.5]nonan-7-yl)-N-(((2R,3R)-2-methyl-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N—(((R)-2,2-dimethyl-5-nitro-3-(tetrahydro-2H-pyran-4-yl)-3,4-dihydro-2H-benzo[b][1,4]oxazin-7-yl)sulfonyl)-4-(2-((R)-4-((5-methoxy-6-((S)-3-methylmorpholino)pyridin-3-yl)methyl)-2-(o-tolyl)piperazin-1-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-((8-methoxy-4,4-dimethyl-3,4-dihydro-2H-pyrano[2,3-c]pyridin-6-yl)methyl)-2-(o-tolyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N—(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)-4-(2-((R)-4-((tetrahydro-2H-pyran-4-yl)methyl)-2-(o-tolyl)piperazin-1-yl)-7-azaspiro[3.5]nonan-7-yl)benzamide,
N—(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)-4-(2-((R)-4-(tetrahydro-2H-pyran-4-yl)-2-(o-tolyl)piperazin-1-yl)-7-azaspiro[3.5]nonan-7-yl)benzamide,
N—(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)-4-(2-((R)-4-((R)-tetrahydro-2H-pyran-3-yl)-2-(o-tolyl)piperazin-1-yl)-7-azaspiro[3.5]nonan-7-yl)benzamide,
N—(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)-4-(2-((R)-4-((S)-tetrahydro-2H-pyran-3-yl)-2-(o-tolyl)piperazin-1-yl)-7-azaspiro[3.5]nonan-7-yl)benzamide,
4-(2-((R)-4-((1r,4R)-4-hydroxy-4-methylcyclohexyl)-2-(o-tolyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-((1s,4S)-4-hydroxy-4-methylcyclohexyl)-2-(o-tolyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N—(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)-4-(2-((R)-4-(1-(tetrahydro-2H-pyran-4-yl)piperidin-4-yl)-2-(o-tolyl)piperazin-1-yl)-7-azaspiro[3.5]nonan-7-yl)benzamide,
4-(2-((R)-4-(3-oxaspiro[5.5]undecan-9-yl)-2-(o-tolyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-((1S,4R)-4-((S)-3-methylmorpholino)cyclohexyl)-2-(o-tolyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-((1R,4S)-4-((S)-3-methylmorpholino)cyclohexyl)-2-(o-tolyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-(4,4-dimethylcyclohexyl)-2-(o-tolyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-(7-oxaspiro[3.5]nonan-2-yl)-2-(o-tolyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-((S)-7-oxaspiro[3.5]nonan-1-yl)-2-(o-tolyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-((R)-7-oxaspiro[3.5]nonan-1-yl)-2-(o-tolyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-(9-hydroxy-9-methylspiro[5.5]undecan-3-yl)-2-(o-tolyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-(7-oxaspiro[3.5]nonan-2-yl)-2-(o-tolyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-((S)-6,7-difluorochroman-4-yl)-2-(o-tolyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-((R)-6,7-difluorochroman-4-yl)-2-(o-tolyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-((R)-7,8-difluorochroman-4-yl)-2-(o-tolyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-((S)-7,8-difluorochroman-4-yl)-2-(o-tolyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-((S)-6,8-difluorochroman-4-yl)-2-(o-tolyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-((R)-6,8-difluorochroman-4-yl)-2-(o-tolyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-((S)-8-methoxy-7-((S)-3-methylmorpholino)-3,4-dihydro-1H-pyrano[4,3-c]pyridin-4-yl)-2-(o-tolyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-((R)-8-methoxy-7-((S)-3-methylmorpholino)-3,4-dihydro-1H-pyrano[4,3-c]pyridin-4-yl)-2-(o-tolyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-((S)-3-methoxy-2-((S)-3-methyl morpholino)-5,8-dihydro-6H-pyrano[3,4-b]pyridin-5-yl)-2-(o-tolyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-((R)-3-methoxy-2-((S)-3-methyl morpholino)-5,8-dihydro-6H-pyrano[3,4-b]pyridin-5-yl)-2-(o-tolyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-((S)-6-fluoro-7-((S)-3-methyl morpholino)-3,4-dihydro-2H-pyrano[2,3-b]pyridin-4-yl)-2-(o-tolyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-((R)-6-fluoro-7-((S)-3-methyl morpholino)-3,4-dihydro-2H-pyrano[2,3-b]pyridin-4-yl)-2-(o-tolyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-((S)-5-fluoro-8-((S)-3-methyl morpholino)-3,4-dihydro-2H-pyrano[2,3-c]pyridin-4-yl)-2-(o-tolyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-((R)-5-fluoro-8-((S)-3-methyl morpholino)-3,4-dihydro-2H-pyrano[2,3-c]pyridin-4-yl)-2-(o-tolyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-((S)-8-fluoro-7-((S)-3-methyl morpholino)-3,4-dihydro-2H-pyrano[3,2-c]pyridin-4-yl)-2-(o-tolyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-((R)-8-fluoro-7-((S)-3-methyl morpholino)-3,4-dihydro-2H-pyrano[3,2-c]pyridin-4-yl)-2-(o-tolyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-((R)-4-methoxy-3-((S)-3-methylmorpholino)-5,6,7,8-tetrahydroisoquinolin-8-yl)-2-(o-tolyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-((S)-4-methoxy-3-((S)-3-methylmorpholino)-5,6,7,8-tetrahydroisoquinolin-8-yl)-2-(o-tolyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-((R)-3-methoxy-2-((S)-3-methylmorpholino)-5,6,7,8-tetrahydroquinolin-5-yl)-2-(o-tolyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-((S)-3-methoxy-2-((S)-3-methylmorpholino)-5,6,7,8-tetrahydroquinolin-5-yl)-2-(o-tolyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N—(((R)-5-nitro-3-(tetrahydro-2H-pyran-4-yl)-3,4-dihydro-2H-benzo[b][1,4]oxazin-7-yl)sulfonyl)-4-(2-((R)-4-phenyl-2-(o-tolyl)piperazin-1-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((2R)-4-(2-(1-methyl-2,6-dioxopiperidin-3-yl)-1,3-dioxoisoindolin-5-yl)-2-(o-tolyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-(1,3-dioxo-2-(tetrahydro-2H-pyran-4-yl)isoindolin-5-yl)-2-(o-tolyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((R)-4-(1,3-dioxo-2-(tetrahydro-2H-pyran-4-yl)isoindolin-4-yl)-2-(o-tolyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
4-(2-((2R)-4-(2-(1-methyl-2,6-dioxopiperidin-3-yl)-1,3-dioxoisoindolin-4-yl)-2-(o-tolyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N—(((R)-5-nitro-3-(tetrahydro-2H-pyran-4-yl)-3,4-dihydro-2H-benzo[b][1,4]oxazin-7-yl)sulfonyl)-4-(2-((R)-4-(pyridin-4-yl)-2-(o-tolyl)piperazin-1-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N—(((R)-5-nitro-3-(tetrahydro-2H-pyran-4-yl)-3,4-dihydro-2H-benzo[b][1,4]oxazin-7-yl)sulfonyl)-4-(2-((R)-4-(pyridin-2-yl)-2-(o-tolyl)piperazin-1-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N—(((R)-5-nitro-3-(tetrahydro-2H-pyran-4-yl)-3,4-dihydro-2H-benzo[b][1,4]oxazin-7-yl)sulfonyl)-4-(2-((R)-4-(pyridin-3-yl)-2-(o-tolyl)piperazin-1-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N—(((R)-5-nitro-3-(tetrahydro-2H-pyran-4-yl)-3,4-dihydro-2H-benzo[b][1,4]oxazin-7-yl)sulfonyl)-4-(2-((R)-4-(pyrimidin-4-yl)-2-(o-tolyl)piperazin-1-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N—(((R)-5-nitro-3-(tetrahydro-2H-pyran-4-yl)-3,4-dihydro-2H-benzo[b][1,4]oxazin-7-yl)sulfonyl)-4-(2-((R)-4-(pyrimidin-5-yl)-2-(o-tolyl)piperazin-1-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N—(((R)-5-nitro-3-(tetrahydro-2H-pyran-4-yl)-3,4-dihydro-2H-benzo[b][1,4]oxazin-7-yl)sulfonyl)-4-(2-((R)-4-(pyrazin-2-yl)-2-(o-tolyl)piperazin-1-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide,
N—(((R)-5-nitro-3-(tetrahydro-2H-pyran-4-yl)-3,4-dihydro-2H-benzo[b][1,4]oxazin-7-yl)sulfonyl)-4-(2-((R)-4-(pyrimidin-2-yl)-2-(o-tolyl)piperazin-1-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide

Compounds of the invention may contain one or more asymmetric carbon atoms. Accordingly, the compounds may exist as diastereomers, enantiomers or mixtures thereof. The syntheses of the compounds may employ racemates, diastereomers or enantiomers as starting materials or as intermediates. Diastereomeric compounds may be separated by chromatographic or crystallization methods. Similarly, enantiomeric mixtures may be separated using the same techniques or others known in the art. Each of the asymmetric carbon atoms may be in the R or S configuration, and both of these configurations are within the scope of the invention.

Compounds having one or more chiral centers can exist in various stereoisomeric forms. Stereoisomers are compounds that differ only in their spatial arrangement. Stereoisomers include all diastereomeric, enantiomeric, and epimeric forms as well as racemates and mixtures thereof.

The term “geometric isomer” refers to cyclic compounds having at least two substituents, wherein the two substituents are both on the same side of the ring (cis) or wherein the substituents are each on opposite sides of the ring (trans). When a disclosed compound is named or depicted by structure without indicating stereochemistry, it is understood that the name or the structure encompasses one or more of the possible stereoisomers, or geometric isomers, or a mixture of the encompassed stereoisomers or geometric isomers.

When a geometric isomer is depicted by name or structure, it is to be understood that the named or depicted isomer exists to a greater degree than another isomer, that is that the geometric isomeric purity of the named or depicted geometric isomer is greater than 50%, such as at least 60%, 70%, 80%, 90%, 99%, or 99.9% pure by weight. Geometric isomeric purity is determined by dividing the weight of the named or depicted geometric isomer in the mixture by the total weight of all of the geomeric isomers in the mixture.

Racemic mixture means 50% of one enantiomer and 50% of is corresponding enantiomer. When a compound with one chiral center is named or depicted without indicating the stereochemistry of the chiral center, it is understood that the name or structure encompasses both possible enantiomeric forms (e.g., both enantiomerically-pure, enantiomerically-enriched or racemic) of the compound. When a compound with two or more chiral centers is named or depicted without indicating the stereochemistry of the chiral centers, it is understood that the name or structure encompasses all possible diasteriomeric forms (e.g., diastereomerically pure, diastereomerically enriched and equimolar mixtures of one or more diastereomers (e.g., racemic mixtures) of the compound.

Enantiomeric and diastereomeric mixtures can be resolved into their component enantiomers or stereoisomers by well-known methods, such as chiral-phase gas chromatography, chiral-phase high performance liquid chromatography, crystallizing the compound as a chiral salt complex, or crystallizing the compound in a chiral solvent. Enantiomers and diastereomers also can be obtained from diastereomerically- or enantiomerically-pure intermediates, reagents, and catalysts by well-known asymmetric synthetic methods.

When a compound is designated by a name or structure that indicates a single enantiomer, unless indicated otherwise, the compound is at least 60%, 70%, 80%, 90%, 99% or 99.9% optically pure (also referred to as “enantiomerically pure”). Optical purity is the weight in the mixture of the named or depicted enantiomer divided by the total weight in the mixture of both enantiomers.

When the stereochemistry of a disclosed compound is named or depicted by structure, and the named or depicted structure encompasses more than one stereoisomer (e.g., as in a diastereomeric pair), it is to be understood that one of the encompassed stereoisomers or any mixture of the encompassed stereoisomers is included. It is to be further understood that the stereoisomeric purity of the named or depicted stereoisomers at least 60%, 70%, 80%, 90%, 99% or 99.9% by weight. The stereoisomeric purity in this case is determined by dividing the total weight in the mixture of the stereoisomers encompassed by the name or structure by the total weight in the mixture of all of the stereoisomers.

A modified compound of any one of such compounds including a modification having an improved (e.g., enhanced, greater) pharmaceutical solubility, stability, bioavailability and/or therapeutic index as compared to the unmodified compound is also contemplated. The examples of modifications include but not limited to the prodrug derivatives, and the deuterium-enriched compounds. For example:
Prodrug derivatives: prodrugs, upon administration to a subject, will converted in vivo into active compounds of the present invention [Nature Reviews of Drug Discovery, 2008, Volume 7, p 255]. It is noted that in many instances, the prodrugs themselves also fall within the scope of the range of compounds according to the present invention. The prodrugs of the compounds of the present invention can be prepared by standard organic reaction, for example, by reacting with a carbamylating agent (e.g., 1,1-acyloxyalkylcarbonochloridate, para-nitrophenyl carbonate, or the like) or an acylating agent. Further examples of methods and strategies of making prodrugs are described in Bioorganic and Medicinal Chemistry Letters, 1994, Vol. 4, p. 1985.
Deuterium-enriched compounds: deuterium (D or ²H) is a stable, non-radioactive isotope of hydrogen and has an atomic weight of 2.0144. Hydrogen naturally occurs as a mixture of the isotopes ^{x}H (hydrogen or protium), D (²H or deuterium), and T (³H or tritium). The natural abundance of deuterium is 0.015%. One of ordinary skill in the art recognizes that in all chemical compounds with a H atom, the H atom actually represents a mixture of H and D, with about 0.015% being D. Thus, compounds with a level of deuterium that has been enriched to be greater than its natural abundance of 0.015%, should be considered unnatural and, as a result, novel over their nonenriched counterparts.

It should be recognized that the compounds of the present invention may be present and optionally administered in the form of salts, and solvates. The invention encompasses any pharmaceutically acceptable salts and solvates of any one of the above-described compounds and modifications thereof. For example, it is within the scope of the present invention to convert the compounds of the present invention into and use them in the form of their pharmaceutically acceptable salts derived from various organic and inorganic acids and bases in accordance with procedures well known in the art.

When the compounds of the present invention possess a free base form, the compounds can be prepared as a pharmaceutically acceptable acid addition salt by reacting the free base form of the compound with a pharmaceutically acceptable inorganic or organic acid, e.g., hydrohalides such as hydrochloride, hydrobromide, hydroiodide; other mineral acids such as sulfate, nitrate, phosphate, etc.; and alkyl and monoarylsulfonates such as ethanesulfonate, toluenesulfonate and benzenesulfonate; and other organic acids and their corresponding salts such as acetate, tartrate, maleate, succinate, citrate, benzoate, salicylate and ascorbate. Further acid addition salts of the present invention include, but are not limited to: adipate, alginate, arginate, aspartate, bisulfate, bisulfite, bromide, butyrate, camphorate, camphorsulfonate, caprylate, chloride, chlorobenzoate, cyclopentanepropionate, digluconate, dihydrogenphosphate, dinitrobenzoate, dodecylsulfate, fumarate, galacterate (from mucic acid), galacturonate, glucoheptaoate, gluconate, glutamate, glycerophosphate, hemisuccinate, hemisulfate, heptanoate, hexanoate, hippurate, 2-hydroxyethanesulfonate, iodide, isethionate, iso-butyrate, lactate, lactobionate, malonate, mandelate, metaphosphate, methanesulfonate, methylbenzoate, monohydrogenphosphate, 2-naphthalenesulfonate, nicotinate, oxalate, oleate, pamoate, pectinate, persulfate, phenylacetate, 3-phenylpropionate, phosphonate and phthalate. It should be recognized that the free base forms will typically differ from their respective salt forms somewhat in physical properties such as solubility in polar solvents, but otherwise the salts are equivalent to their respective free base forms for the purposes of the present invention.

When the compounds of the present invention possess a free acid form, a pharmaceutically acceptable base addition salt can be prepared by reacting the free acid form of the compound with a pharmaceutically acceptable inorganic or organic base. Examples of such bases are alkali metal hydroxides including potassium, sodium and lithium hydroxides; alkaline earth metal hydroxides such as barium and calcium hydroxides; alkali metal alkoxides, e.g., potassium ethanolate and sodium propanolate; and various organic bases such as ammonium hydroxide, piperidine, diethanolamine and N-methylglutamine. Also included are the aluminum salts of the compounds of the present invention. Further base salts of the present invention include, but are not limited to: copper, ferric, ferrous, lithium, magnesium, manganic, manganous, potassium, sodium and zinc salts. Organic base salts include, but are not limited to, salts of primary, secondary and tertiary amines, substituted amines including naturally occurring substituted amines, cyclic amines and basic ion exchange resins, e.g., arginine, betaine, caffeine, chloroprocaine, choline, N,N′-dibenzylethylenediamine (benzathine), dicyclohexylamine, diethanolamine, 2-diethylaminoethanol, 2-dimethylaminoethanol, ethanolamine, ethylenediamine, N-ethylmorpholine, N-ethylpiperidine, glucamine, glucosamine, histidine, hydrabamine, iso-propylamine, lidocaine, lysine, meglumine, N-methyl-D-glucamine, morpholine, piperazine, piperidine, polyamine resins, procaine, purines, theobromine, triethanolamine, triethylamine, trimethylamine, tripropylamine and tris-(hydroxymethyl)-methylamine (tromethamine). It should be recognized that the free acid forms will typically differ from their respective salt forms somewhat in physical properties such as solubility in polar solvents, but otherwise the salts are equivalent to their respective free acid forms for the purposes of the present invention.

In one aspect, a pharmaceutically acceptable salt is a hydrochloride salt, hydrobromide salt, methanesulfonate, toluenesulfonate, acetate, fumarate, sulfate, bisulfate, succinate, citrate, phosphate, maleate, nitrate, tartrate, benzoate, biocarbonate, carbonate, sodium hydroxide salt, calcium hydroxide salt, potassium hydroxide salt, tromethamine salt, or mixtures thereof.

Compounds of the present invention that comprise tertiary nitrogen-containing groups may be quaternized with such agents as (C₁₋₄) alkyl halides, e.g., methyl, ethyl, iso-propyl and tert-butyl chlorides, bromides and iodides; di-(C₁₋₄) alkyl sulfates, e.g., dimethyl, diethyl and diamyl sulfates; alkyl halides, e.g., decyl, dodecyl, lauryl, myristyl and stearyl chlorides, bromides and iodides; and aryl (C₁₋₄) alkyl halides, e.g., benzyl chloride and phenethyl bromide. Such salts permit the preparation of both water- and oil-soluble compounds of the invention.

Amine oxides, also known as amine-N-oxide and N-oxide, of anti-cancer agents with tertiary nitrogen atoms have been developed as prodrugs [Mol Cancer Therapy. 2004 March; 3(3):233-44]. Compounds of the present invention that comprise tertiary nitrogen atoms may be oxidized by such agents as hydrogen peroxide (H₂O₂), Caro's acid or peracids like meta-Chloroperoxybenzoic acid (mCPBA) to from amine oxide.

The compounds disclosed therein are bcl-2 inhibitors. The pharmaceutical composition of the present invention comprises one or more bcl-2 inhibitors, or a pharmaceutically acceptable salt thereof, and a pharmaceutically acceptable carrier or diluent.

“Pharmaceutically acceptable carrier” and “pharmaceutically acceptable diluent” refer to a substance that aids the formulation and/or administration of an active agent to and/or absorption by a subject and can be included in the compositions of the present disclosure without causing a significant adverse toxicological effect on the subject. Non-limiting examples of pharmaceutically acceptable carriers and/or diluents include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethycellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with or interfere with the activity of the compounds provided herein. One of ordinary skill in the art will recognize that other pharmaceutical excipients are suitable for use with disclosed compounds.

The pharmaceutical compositions of the present invention optionally include one or more pharmaceutically acceptable carriers and/or diluents therefor, such as lactose, starch, cellulose and dextrose. Other excipients, such as flavoring agents; sweeteners; and preservatives, such as methyl, ethyl, propyl and butyl parabens, can also be included. More complete listings of suitable excipients can be found in the Handbook of Pharmaceutical Excipients (5^{th} Ed., Pharmaceutical Press (2005)). A person skilled in the art would know how to prepare formulations suitable for various types of administration routes. Conventional procedures and ingredients for the selection and preparation of suitable formulations are described, for example, in Remington's Pharmaceutical Sciences (2003-20th edition) and in The United States Pharmacopeia: The National Formulary (USP 24 NF19) published in 1999. The carriers, diluents and/or excipients are “acceptable” in the sense of being compatible with the other ingredients of the pharmaceutical composition and not deleterious to the recipient thereof.

The pharmaceutical compositions of the present invention may further comprise other conventional pharmaceutically inactive agents. Any inert excipient that is commonly used as a carrier or diluent may be used in compositions of the present invention, such as sugars, polyalcohols, soluble polymers, salts and lipids. Sugars and polyalcohols which may be employed include, without limitation, lactose, sucrose, mannitol, and sorbitol. Illustrative of the soluble polymers which may be employed are polyoxyethylene, poloxamers, polyvinylpyrrolidone, and dextran. Useful salts include, without limitation, sodium chloride, magnesium chloride, and calcium chloride. Lipids which may be employed include, without limitation, fatty acids, glycerol fatty acid esters, glycolipids, and phospholipids.

In addition, the pharmaceutical compositions of the present invention may further comprise binders (e.g., acacia, cornstarch, gelatin, carbomer, ethyl cellulose, guar gum, hydroxypropyl cellulose, hydroxypropyl methyl cellulose, povidone), disintegrating agents (e.g., cornstarch, potato starch, alginic acid, silicon dioxide, croscarmellose sodium, crospovidone, guar gum, sodium starch glycolate, Primogel), buffers (e.g., tris-HCL, acetate, phosphate) of various pH and ionic strength, additives such as albumin or gelatin to prevent absorption to surfaces, detergents (e.g., Tween 20, Tween 80, Pluronic F68, bile acid salts), protease inhibitors, surfactants (e.g., sodium lauryl sulfate), permeation enhancers, solubilizing agents (e.g., glycerol, polyethylene glycerol, cyclodextrins), a glidant (e.g., colloidal silicon dioxide), anti-oxidants (e.g., ascorbic acid, sodium metabisulfite, butylated hydroxyanisole), stabilizers (e.g., hydroxypropyl cellulose, hydroxypropylmethyl cellulose), viscosity increasing agents (e.g., carbomer, colloidal silicon dioxide, ethyl cellulose, guar gum), sweeteners (e.g., sucrose, aspartame, citric acid), flavoring agents (e.g., peppermint, methyl salicylate, or orange flavoring), preservatives (e.g., Thimerosal, benzyl alcohol, parabens), lubricants (e.g., stearic acid, magnesium stearate, polyethylene glycol, sodium lauryl sulfate), flow-aids (e.g., colloidal silicon dioxide), plasticizers (e.g., diethyl phthalate, triethyl citrate), emulsifiers (e.g., carbomer, hydroxypropyl cellulose, sodium lauryl sulfate, methyl cellulose, hydroxyethyl cellulose, carboxymethylcellulose sodium), polymer coatings (e.g., poloxamers or poloxamines), coating and film forming agents (e.g., ethyl cellulose, acrylates, polymethacrylates) and/or adjuvants.

In one embodiment, the pharmaceutical compositions are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

Additionally, the invention encompasses pharmaceutical compositions comprising any solid or liquid physical form of the compound of the invention. For example, the compounds can be in a crystalline form, in amorphous form, and have any particle size. The particles may be micronized, or may be agglomerated, particulate granules, powders, oils, oily suspensions or any other form of solid or liquid physical form.

When compounds according to the present invention exhibit insufficient solubility, methods for solubilizing the compounds may be used. Such methods are known to those of skill in this art, and include, but are not limited to, pH adjustment and salt formation, using co-solvents, such as ethanol, propylene glycol, polyethylene glycol (PEG) 300, PEG 400, DMA (10-30%), DMSO (10-20%), NMP (10-20%), using surfactants, such as polysorbate 80, polysorbate 20 (1-10%), cremophor EL, Cremophor RH40, Cremophor RH60 (5-10%), Pluronic F68/Poloxamer 188 (20-50%), Solutol HS15 (20-50%), Vitamin E TPGS, and d-α-tocopheryl PEG 1000 succinate (20-50%), using complexation such as HPβCD and SBEβCD (10-40%), and using advanced approaches such as micelle, addition of a polymer, nanoparticle suspensions, and liposome formation.

A wide variety of administration methods may be used in conjunction with the compounds of the present invention. Compounds of the present invention may be administered or coadministered orally, parenterally, intraperitoneally, intravenously, intraarterially, transdermally, sublingually, intramuscularly, rectally, transbuccally, intranasally, liposomally, via inhalation, vaginally, intraoccularly, via local delivery (for example by catheter or stent), subcutaneously, intraadiposally, intraarticularly, or intrathecally. The compounds according to the invention may also be administered or coadministered in slow release dosage forms. Compounds may be in gaseous, liquid, semi-liquid or solid form, formulated in a manner suitable for the route of administration to be used. For oral administration, suitable solid oral formulations include tablets, capsules, pills, granules, pellets, sachets and effervescent, powders, and the like. Suitable liquid oral formulations include solutions, suspensions, dispersions, emulsions, oils and the like. For parenteral administration, reconstitution of a lyophilized powder is typically used.

As used herein, “acyl” means a carbonyl containing substituent represented by the formula —C(O)—R in which R is H, alkyl, a carbocycle, a heterocycle, carbocycle-substituted alkyl or heterocycle-substituted alkyl wherein the alkyl, alkoxy, carbocycle and heterocycle are as defined herein. Acyl groups include alkanoyl (e.g. acetyl), aroyl (e.g. benzoyl), and heteroaroyl.

“Aliphatic” means a moiety characterized by a straight or branched chain arrangement of constituent carbon atoms and may be saturated or partially unsaturated with one or more double or triple bonds.

The term “alkyl” refers to a straight or branched hydrocarbon containing 1-20 carbon atoms (e.g., C₁-C₁₀, C₁-C₆). Examples of alkyl include, but are not limited to, methyl, methylene, ethyl, ethylene, n-propyl, i-propyl, n-butyl, i-butyl, and t-butyl. Preferably, the alkyl group has one to ten carbon atoms. More preferably, the alkyl group has one to four carbon atoms.

The term “alkenyl” refers to a straight or branched hydrocarbon containing 2-20 carbon atoms (e.g., C₂-C₁₀, C₂-C₆) and one or more double bonds. Examples of alkenyl include, but are not limited to, ethenyl, propenyl, and allyl. Preferably, the alkylene group has two to ten carbon atoms. More preferably, the alkylene group has two to four carbon atoms.

The term “alkynyl” refers to a straight or branched hydrocarbon containing 2-20 carbon atoms (e.g., C₂—C₁₀, C₂—C) and one or more triple bonds. Examples of alkynyl include, but are not limited to, ethynyl, 1-propynyl, 1- and 2-butynyl, and 1-methyl-2-butynyl. Preferably, the alkynyl group has two to ten carbon atoms. More preferably, the alkynyl group has two to four carbon atoms.

The term “alkylamino” refers to an —N(R)-alkyl in which R can be H, alkyl, alkenyl, alkynyl, cycloalkyl, cycloalkenyl, heterocycloalkyl, heterocycloalkenyl, aryl, or heteroaryl.

“Alkoxy” means an oxygen moiety having a further alkyl substituent.

“Alkoxycarbonyl” means an alkoxy group attached to a carbonyl group.

“Oxoalkyl” means an alkyl, further substituted with a carbonyl group. The carbonyl group may be an aldehyde, ketone, ester, amide, acid or acid chloride.

The term “cycloalkyl” refers to a saturated hydrocarbon ring system having 3 to 30 carbon atoms (e.g., C₃-C₁₂, C₃-C₈, C₃-C₆). Examples of cycloalkyl include, but are not limited to, cyclopropyl, cyclobutyl, cyclopentyl, cyclohexyl, cycloheptyl, and cyclooctyl.

The term “cycloalkenyl” refers to a non-aromatic hydrocarbon ring system having 3 to 30 carbons (e.g., C₃-C₁₂) and one or more double bonds. Examples include cyclopentenyl, cyclohexenyl, and cycloheptenyl.

Spirocycloalkyl refers to a compound comprising two saturated cyclic alkyl rings sharing only one common atom (also known as a spiro atom), with no heteroatom and no unsaturated bonds on any of the rings. In one embodiment, the spiroalkyl is bicyclic. In another embodiment, the spiroalikyl has more than two cycles. In certain embodiments, the spiroalkyl compound is a polyspiro compound connected by two or more spiroatoms making up three or more rings. In certain embodiments, one of the rings of the bicyclic spiroalkyl has 3, 4, 5, 6, 7, or 8 atoms, including the common spito atom. In one embodiment, the spiroalkyl is a 5 to 20 membered, 5 to 14 membered, or 5 to 10 membered polycyclic spiroalkyl group. Representative examples of spiroalkyl include, but are not limited to the following groups:

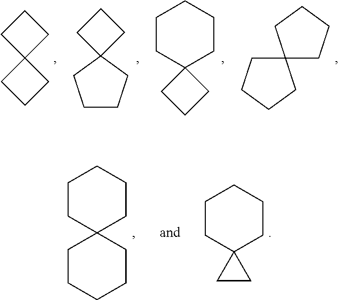

The term “fused-carbocyclic” refers to a polycyclic cyclyl group, wherein each ring in the group shares an adjacent pair of carbon atoms with another ring in the group, wherein one or more rings can contain one or more double bonds. In certain embodiments, the fused heterocyclyl is bicyclic. In certain embodiments, the fused-carbocyclic contains more than two rings, at least two of which share an adjacent pair of atoms. In one embodiment, the fused-carbocyclic is a 5 to 20 membered, 5 to 16 membered, or 5 to 10 membered polycyclic cyclyl group. Representative examples of fused-carbocyclic include, but are not limited to the following groups:

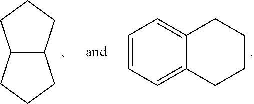 [SMILES: C1CC2CCCC2C1 ; c1ccc2c(c1)CCCC2]

The term “bridged-carbocyclic” refers to a group having at least two rings sharing three or more common ring atoms, separating the two bridgehead atoms by a bridge containing at least one atom. The bridgehead atoms are the atoms from which three bonds radiate and where the rings meet. The rings of the bridged carbocyclyl can have one or more double bonds. In one embodiment, the bridged carbocyclyl is bicyclic. In one embodiment, the bridged carbocyclyl is a 5 to 20 membered, 5 to 16 membered, or 5 to 10 membered polycyclic carbocyclyl group. Representative examples of bridged carbocyclyl include, but are not limited to the following groups:

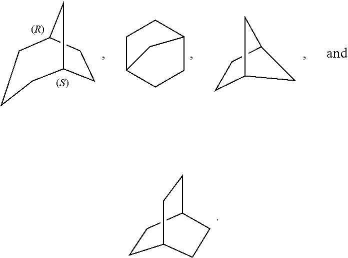

The term “heterocycloalkyl” refers to a nonaromatic 5-8 membered monocyclic, 8-12 membered bicyclic, 11-14 membered tricyclic, or 14-20 membered tetracyclic ring system having one or more heteroatoms (such as O, N, S, P, or Se). Examples of heterocycloalkyl groups include, but are not limited to, piperazinyl, pyrrolidinyl, dioxanyl, morpholinyl, and tetrahydrofuranyl.

The term “heterocycloalkenyl” refers to a nonaromatic 5-8 membered monocyclic, 8-12 membered bicyclic, 11-14 membered tricyclic, or 14-20 membered tetracyclic ring system having one or more heteroatoms (such as O, N, S, P, or Se) and one or more double bonds.

Spiroheterocyclyl refers to a compound comprising two non-saturated rings sharing only one common atom (also known as a spiro atom), with at least one heteroatom on one of the two rings, such as a polycyclic heterocyclyl group with rings connected through one common carbon atom. The common atom can be carbon (C), silicon, or nitrogen (such as a positively charged quaternary nitrogen atom). The heteroatoms can comprise nitrogen, quaternary nitrogen, oxidized nitrogen (e.g., NO), oxygen, silicon, and sulfur, including sulfoxide and sulfone, and the remaining ring atoms are C. In addition, one or more of the rings may contain one or more double bonds. In one embodiment, the spiro heterocyclyl is bicyclic, with heteroatom(s) on either one or both cycles. In certain embodiments, one of the rings of the bicyclic spiro heterocyclyl has 3, 4, 5, 6, 7, or 8 atoms, including the common spito atom. In certain embodiments, the spiro heterocyclic compound is a polyspiro compound connected by two or more spiroatoms making up three or more rings. In one embodiment, the spiro heterocyclyl is a 5 to 20 membered, 5 to 14 membered, or 5 to 10 membered polycyclic heterocyclyl group. Representative examples of spiro heterocyclyl include, but are not limited to the following groups:

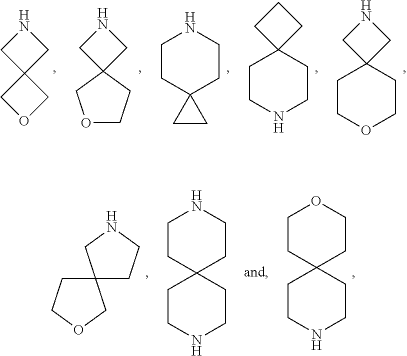

Fused heterocyclyl refers to a polycyclic heterocyclyl group, wherein each ring in the group shares an adjacent pair of atoms (such as carbon atoms) with another ring in the group, wherein one or more rings can contain one or more double bonds, and wherein said rings have one or more heteroatoms, which can be nitrogen, quaternary nitrogen, oxidized nitrogen (e.g., NO), oxygen, and sulfur, including sulfoxide and sulfone, and the remaining ring atoms are C. In certain embodiments, the fused heterocyclyl is bicyclic. In certain embodiments, the fused heterocyclyl contains more than two rings, at least two of which share an adjacent pair of atoms. In one embodiment, the fused heterocyclyl is a 5 to 20 membered, 5 to 16 membered, or 5 to 10 membered polycyclic heterocyclyl group. Representative examples of fused heterocyclyl include, but are not limited to the following groups:

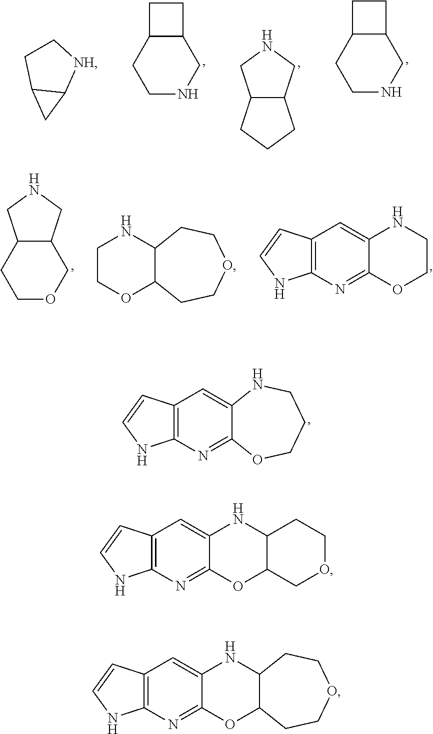

Bridged heterocyclyl refers to a compound having at least two rings sharing three or more common ring atoms, separating the two bridgehead atoms by a bridge containing at least one atom, wherein at least one ring atom is a heteroatom. The bridgehead atoms are the atoms from which three bonds radiate and where the rings meet. The rings of the bridged heterocyclyl can have one or more double bonds, and the ring heteroatom(s) can be nitrogen, quaternary nitrogen, oxidized nitrogen (e.g., NO), oxygen, and sulfur, including sulfoxide and sulfone as ring atoms, while the remaining ring atoms are C. In one embodiment, the bridged heterocyclyl is bicyclic. In one embodiment, the bridged heterocyclyl is a 5 to 20 membered, 5 to 16 membered, or 5 to 10 membered polycyclic heterocyclyl group. Representative examples of bridged heterocyclyl include, but are not limited to the following groups:

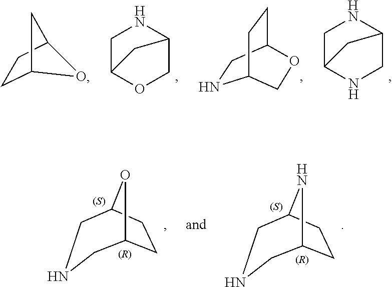

The term “aryl” refers to a 6-carbon monocyclic, 10-carbon bicyclic, 14-carbon tricyclic aromatic ring system. Examples of aryl groups include, but are not limited to, phenyl, naphthyl, and anthracenyl.

The term “heteroaryl” refers to an aromatic 5-8 membered monocyclic, 8-12 membered bicyclic, or 11-14 membered tricyclic ring system having one or more heteroatoms (such as O, N, S, P, or Se). Examples of heteroaryl groups include pyridyl, furyl, imidazolyl, benzimidazolyl, pyrimidinyl, thienyl, quinolinyl, indolyl, and thiazolyl.

Alkyl, alkenyl, alkynyl, cycloalkyl, heterocycloalkyl, cycloalkenyl, heterocycloalkenyl, alkylamino, spiroheterocyclyl, fused heterocyclyl, bridged heterocyclyl, aryl, and heteroaryl mentioned above include both substituted and unsubstituted moieties. Possible substituents on alkylamino, cycloalkyl, heterocycloalkyl, cycloalkenyl, heterocycloalkenyl, aryl, and heteroaryl include, but are not limited to, C₁-C₁₀ alkyl, C₂-C₁₀ alkenyl, C₂-C₁₀ alkynyl, C₃-C₂₀ cycloalkyl, C₃-C₂₀ cycloalkenyl, C₁-C₂₀ heterocycloalkyl, C₁-C₂₀ heterocycloalkenyl, C₁-C₁₀ alkoxy, aryl, aryloxy, heteroaryl, heteroaryloxy, amino, C₁-C₁₀ alkylamino, arylamino, hydroxy, halo, oxo (O=), thioxo (S=), thio, silyl, C₁-C₁₀ alkylthio, arylthio, C₁-C₁₀ alkylsulfonyl, arylsulfonyl, acylamino, aminoacyl, aminothioacyl, amidino, mercapto, amido, thioureido, thiocyanato, sulfonamido, guanidine, ureido, cyano, nitro, acyl, thioacyl, acyloxy, carbamido, carbamyl, carboxyl, and carboxylic ester. On the other hand, possible substituents on alkyl, alkenyl, or alkynyl include all of the above-recited substituents except C₁-C₁₀ alkyl. Cycloalkyl, cycloalkenyl, heterocycloalkyl, heterocycloalkenyl, aryl, and heteroaryl can also be fused with each other.

“Amino” means a nitrogen moiety having two further substituents where each substituent has a hydrogen or carbon atom alpha bonded to the nitrogen. Unless indicated otherwise, the compounds of the invention containing amino moieties may include protected derivatives thereof. Suitable protecting groups for amino moieties include acetyl, tert-butoxycarbonyl, benzyloxycarbonyl, and the like.

“Aromatic” means a moiety wherein the constituent atoms make up an unsaturated ring system, all atoms in the ring system are sp2 hybridized and the total number of pi electrons is equal to 4n+2. An aromatic ring may be such that the ring atoms are only carbon atoms or may include carbon and non-carbon atoms (see Heteroaryl).

“Carbamoyl” means the radical —OC(O)NRₐR_{b} where Rₐ and R_{b} are each independently two further substituents where a hydrogen or carbon atom is alpha to the nitrogen. It is noted that carbamoyl moieties may include protected derivatives thereof. Examples of suitable protecting groups for carbamoyl moieties include acetyl, tert-butoxycarbonyl, benzyloxycarbonyl, and the like. It is noted that both the unprotected and protected derivatives fall within the scope of the invention.

“Carbonyl” means the radical —C(O)—. It is noted that the carbonyl radical may be further substituted with a variety of substituents to form different carbonyl groups including acids, acid halides, amides, esters, and ketones.

“Carboxy” means the radical —C(O)O—. It is noted that compounds of the invention containing carboxy moieties may include protected derivatives thereof, i.e., where the oxygen is substituted with a protecting group. Suitable protecting groups for carboxy moieties include benzyl, tert-butyl, and the like.

“Cyano” means the radical —CN.

“Formyl” means the radical —CH═O.

“Formimino” means the radical —HC≡NH.

“Halo” means fluoro, chloro, bromo or iodo.

“Halo-substituted alkyl”, as an isolated group or part of a larger group, means “alkyl” substituted by one or more “halo” atoms, as such terms are defined in this Application. Halo-substituted alkyl includes haloalkyl, dihaloalkyl, trihaloalkyl, perhaloalkyl and the like.

“Hydroxy” means the radical —OH.

“Imine derivative” means a derivative comprising the moiety —C(═NR)—, wherein R comprises a hydrogen or carbon atom alpha to the nitrogen.

“Isomers” mean any compound having identical molecular formulae but differing in the nature or sequence of bonding of their atoms or in the arrangement of their atoms in space. Isomers that differ in the arrangement of their atoms in space are termed “stereoisomers.” Stereoisomers that are not mirror images of one another are termed “diastereomers” and stereoisomers that are nonsuperimposable mirror images are termed “enantiomers” or sometimes “optical isomers.” A carbon atom bonded to four nonidentical substituents is termed a “chiral center.” A compound with one chiral center has two enantiomeric forms of opposite chirality. A mixture of the two enantiomeric forms is termed a “racemic mixture.”

“Nitro” means the radical —NO₂.

“Protected derivatives” means derivatives of compounds in which a reactive site are blocked with protecting groups. Protected derivatives are useful in the preparation of pharmaceuticals or in themselves may be active as inhibitors. A comprehensive list of suitable protecting groups can be found in T. W. Greene, Protecting Groups in Organic Synthesis, 3rd edition, Wiley & Sons, 1999.

The term “substituted” means that an atom or group of atoms has replaced hydrogen as the substituent attached to another group. For aryl and heteroaryl groups, the term “substituted” refers to any level of substitution, namely mono-, di-, tri-, tetra-, or penta-substitution, where such substitution is permitted. The substituents are independently selected, and substitution may be at any chemically accessible position. The term “unsubstituted” means that a given moiety may consist of only hydrogen substituents through available valencies (unsubstituted).

If a functional group is described as being “optionally substituted,” the function group may be either (1) not substituted, or (2) substituted. If a carbon of a functional group is described as being optionally substituted with one or more of a list of substituents, one or more of the hydrogen atoms on the carbon (to the extent there are any) may separately and/or together be replaced with an independently selected optional substituent.

“Sulfide” means —S—R wherein R is H, alkyl, carbocycle, heterocycle, carbocycloalkyl or heterocycloalkyl. Particular sulfide groups are mercapto, alkylsulfide, for example methylsulfide (—S-Me); arylsulfide, e.g., phenylsulfide; aralkylsulfide, e.g., benzylsulfide.

“Sulfinyl” means the radical —S(O)—. It is noted that the sulfinyl radical may be further substituted with a variety of substituents to form different sulfinyl groups including sulfinic acids, sulfinamides, sulfinyl esters, and sulfoxides.

“Sulfonyl” means the radical —S(O)(O)—. It is noted that the sulfonyl radical may be further substituted with a variety of substituents to form different sulfonyl groups including sulfonic acids, sulfonamides, sulfonate esters, and sulfones.

“Thiocarbonyl” means the radical —C(S)—. It is noted that the thiocarbonyl radical may be further substituted with a variety of substituents to form different thiocarbonyl groups including thioacids, thioamides, thioesters, and thioketones.

“Animal” includes humans, non-human mammals (e.g., non-human primates, rodents, mice, rats, hamsters, dogs, cats, rabbits, cattle, horses, sheep, goats, swine, deer, and the like) and non-mammals (e.g., birds, and the like).

“Bioavailability” as used herein is the fraction or percentage of an administered dose of a drug or pharmaceutical composition that reaches the systemic circulation intact. In general, when a medication is administered intravenously, its bioavailability is 100%. However, when a medication is administered via other routes (e.g., orally), its bioavailability decreases (e.g., due to incomplete absorption and first-pass metabolism). Methods to improve the bioavailability include prodrug approach, salt synthesis, particle size reduction, complexation, change in physical form, solid dispersions, spray drying, and hot-melt extrusion.

“Disease” specifically includes any unhealthy condition of an animal or part thereof and includes an unhealthy condition that may be caused by, or incident to, medical or veterinary therapy applied to that animal, i.e., the “side effects” of such therapy.

“Pharmaceutically acceptable” means that which is useful in preparing a pharmaceutical composition that is generally safe, non-toxic and neither biologically nor otherwise undesirable and includes that which is acceptable for veterinary use as well as human pharmaceutical use.

“Pharmaceutically acceptable salts” means organic or inorganic salts of compounds of the present invention which are pharmaceutically acceptable, as defined above, and which possess the desired pharmacological activity. Such salts include acid addition salts formed with inorganic acids, or with organic acids. Pharmaceutically acceptable salts also include base addition salts which may be formed when acidic protons present are capable of reacting with inorganic or organic bases. Exemplary salts include, but are not limited, to sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucuronate, saccharate, formate, benzoate, glutamate, methanesulfonate “mesylate,” ethanesulfonate, benzenesulfonate, p-toluenesulfonate, pamoate (i.e., 1,1′-methylene-bis-(2-hydroxy-3-naphthoate)) salts, alkali metal (e.g., sodium and potassium) salts, alkaline earth metal (e.g., magnesium) salts, and ammonium salts. A pharmaceutically acceptable salt may involve the inclusion of another molecule such as an acetate ion, a succinate ion or other counter ion. The counter ion may be any organic or inorganic moiety that stabilizes the charge on the parent compound. Furthermore, a pharmaceutically acceptable salt may have more than one charged atom in its structure. Instances where multiple charged atoms are part of the pharmaceutically acceptable salt can have multiple counter ions. Hence, a pharmaceutically acceptable salt can have one or more charged atoms and/or one or more counter ion.

“Pharmacophore,” as defined by The International Union of Pure and Applied Chemistry, is an ensemble of steric and electronic features that is necessary to ensure the optimal supramolecular interactions with a specific biological target and to trigger (or block) its biological response. For example, Camptothecin is the pharmacophore of the well known drug topotecan and irinotecan. Mechlorethamine is the pharmacophore of a list of widely used nitrogen mustard drugs like Melphalan, Cyclophosphamide, Bendamustine, and so on.

“Prodrug” means a compound that is convertible in vivo metabolically into an active pharmaceutical according to the present invention. For example, an inhibitor comprising a hydroxyl group may be administered as an ester that is converted by hydrolysis in vivo to the hydroxyl compound.

“Stability” in general refers to the length of time a drug retains its properties without loss of potency. Sometimes this is referred to as shelf life. Factors affecting drug stability include, among other things, the chemical structure of the drug, impurity in the formulation, pH, moisture content, as well as environmental factors such as temperature, oxidization, light, and relative humidity. Stability can be improved by providing suitable chemical and/or crystal modifications (e.g., surface modifications that can change hydration kinetics; different crystals that can have different properties), excipients (e.g., anything other than the active substance in the dosage form), packaging conditions, storage conditions, etc.

“Therapeutically effective amount” of a composition described herein is meant an amount of the composition which confers a therapeutic effect on the treated subject, at a reasonable benefit/risk ratio applicable to any medical treatment. The therapeutic effect may be objective (i.e., measurable by some test or marker) or subjective (i.e., subject gives an indication of or feels an effect). An effective amount of the composition described above may range from about 0.1 mg/kg to about 500 mg/kg, preferably from about 0.2 to about 50 mg/kg. Effective doses will also vary depending on route of administration, as well as the possibility of co-usage with other agents. It will be understood, however, that the total daily usage of the compositions of the present invention will be decided by the attending physician within the scope of sound medical judgment. The specific therapeutically effective dose level for any particular patient will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or contemporaneously with the specific compound employed; and like factors well known in the medical arts.

As used herein, the term “treating” refers to administering a compound to a subject that has a neoplastic or immune disorder, or has a symptom of or a predisposition toward it, with the purpose to cure, heal, alleviate, relieve, alter, remedy, ameliorate, improve, or affect the disorder, the symptoms of or the predisposition toward the disorder. The term “an effective amount” refers to the amount of the active agent that is required to confer the intended therapeutic effect in the subject. Effective amounts may vary, as recognized by those skilled in the art, depending on route of administration, excipient usage, and the possibility of co-usage with other agents.

A “subject” refers to a human and a non-human animal. Examples of a non-human animal include all vertebrates, e.g., mammals, such as non-human primates (particularly higher primates), dog, rodent (e.g., mouse or rat), guinea pig, cat, and non-mammals, such as birds, amphibians, reptiles, etc. In a preferred embodiment, the subject is a human. In another embodiment, the subject is an experimental animal or animal suitable as a disease model.

“Combination therapy” includes the administration of the subject compounds of the present invention in further combination with other biologically active ingredients (such as, but not limited to, a second and different antineoplastic agent) and non-drug therapies (such as, but not limited to, surgery or radiation treatment). For instance, the compounds of the invention can be used in combination with other pharmaceutically active compounds, or non-drug therapies, preferably compounds that are able to enhance the effect of the compounds of the invention. The compounds of the invention can be administered simultaneously (as a single preparation or separate preparation) or sequentially to the other therapies. In general, a combination therapy envisions administration of two or more drugs/treatments during a single cycle or course of therapy.

In one embodiment, the compounds of the invention are administered in combination with one or more of traditional chemotherapeutic agents. The traditional chemotherapeutic agents encompass a wide range of therapeutic treatments in the field of oncology. These agents are administered at various stages of the disease for the purposes of shrinking tumors, destroying remaining cancer cells left over after surgery, inducing remission, maintaining remission and/or alleviating symptoms relating to the cancer or its treatment. Examples of such agents include, but are not limited to, alkylating agents such as Nitrogen Mustards (e.g., Bendamustine, Cyclophosphamide, Melphalan, Chlorambucil, Isofosfamide), Nitrosureas (e.g., Carmustine, Lomustine and Streptozocin), ethylenimines (e.g., thiotepa, hexamethylmelanine), Alkylsulfonates (e.g., Busulfan), Hydrazines and Triazines (e.g., Altretamine, Procarbazine, Dacarbazine and Temozolomide), and platinum based agents (e.g., Carboplatin, Cisplatin, and Oxaliplatin); plant alkaloids such as Podophyllotoxins (e.g., Etoposide and Tenisopide), Taxanes (e.g., Paclitaxel and Docetaxel), Vinca alkaloids (e.g., Vincristine, Vinblastine and Vinorelbine); anti-tumor antibiotics such as Chromomycins (e.g., Dactinomycin and Plicamycin), Anthracyclines (e.g., Doxorubicin, Daunorubicin, Epirubicin, Mitoxantrone, and Idarubicin), and miscellaneous antibiotics such as Mitomycin and Bleomycin; anti-metabolites such as folic acid antagonists (e.g., Methotrexate), pyrimidine antagonists (e.g., 5-Fluorouracil, Foxuridine, Cytarabine, Capecitabine, and Gemcitabine), purine antagonists (e.g., 6-Mercaptopurine and 6-Thioguanine) and adenosine deaminase inhibitors (e.g., Cladribine, Fludarabine, Nelarabine and Pentostatin); topoisomerase inhibitors such as topoisomerase I inhibitors(Topotecan, Irinotecan), topoisomerase II inhibitors (e.g., Amsacrine, Etoposide, Etoposide phosphate, Teniposide), and miscellaneous anti-neoplastics such as ribonucleotide reductase inhibitors (Hydroxyurea), adrenocortical steroid inhibitor (Mitotane), anti-microtubule agents (Estramustine), and retinoids (Bexarotene, Isotretinoin, Tretinoin (ATRA).

In one aspect of the invention, the compounds may be administered in combination with one or more targeted anti-cancer agents that modulate protein kinases involved in various disease states. Examples of such kinases may include, but are not limited ABL1, ABL2/ARG, ACK1, AKT1, AKT2, AKT3, ALK, ALK1/ACVRL1, ALK2/ACVR1, ALK4/ACVR1B, ALK5/TGFBR1, ALK6/BMPR1B, AMPK(A1/B1/G1), AMPK(A1/B1/G2), AMPK(A1/B1/G3), AMPK(A1/B2/G1), AMPK(A2/B1/G1), AMPK(A2/B2/G1), AMPK(A2/B2/G2), ARAF, ARK5/NUAK1, ASK1/MAP3K5, ATM, Aurora A, Aurora B, Aurora C, AXL, BLK, BMPR2, BMX/ETK, BRAF, BRK, BRSK1, BRSK2, BTK, CAMK1a, CAMK1b, CAMK1d, CAMK1g, CAMKlla, CAMKllb, CAMKlld, CAMKllg, CAMK4, CAMKK1, CAMKK2, CDC7-DBF4, CDK1-cyclin A, CDK1-cyclin B, CDK1-cyclin E, CDK2-cyclin A, CDK2-cyclin A1, CDK2-cyclin E, CDK3-cyclin E, CDK4-cyclin D1, CDK4-cyclin D3, CDK5-p25, CDK5-p35, CDK6-cyclin D1, CDK6-cyclin D3, CDK7-cyclin H, CDK9-cyclin K, CDK9-cyclin T1, CHK1, CHK2, CK1a1, CK1d, CK1epsilon, CK1g1, CK1g2, CK1g3, CK2a, CK2a2, c-KIT, CLK1, CLK2, CLK3, CLK4, c-MER, c-MET, COT1/MAP3K8, CSK, c-SRC, CSF1R, CTK/MATK, DAPK1, DAPK2, DCAMKL1, DCAMKL2, DDR1, DDR2, DLK/MAP3K12, DMPK, DMPK2/CDC42BPG, DNA-PK, DRAK1/STK17A, DYRK1/DYRK1A, DYRK1B, DYRK2, DYRK3, DYRK4, EEF2K, EGFR, EIF2AK1, EIF2AK2, EIF2AK3, EIF2AK4/GCN2, EPHA1, EPHA2, EPHA3, EPHA4, EPHA5, EPHA6, EPHA7, EPHA8, EPHB1, EPHB2, EPHB3, EPHB4, ERBB2/HER2, ERBB4/HER4, ERK1/MAPK3, ERK2/MAPK1, ERK5/MAPK7, FAK/PTK2, FER, FES/FPS, FGFR1, FGFR2, FGFR3, FGFR4, FGR, FLT1NEGFR1, FLT3, FLT4NEGFR3, FMS, FRK/PTK5, FYN, GCK/MAP4K2, GRK1, GRK2, GRK3, GRK4, GRK5, GRK6, GRK7, GSK3a, GSK3b, Haspin, HCK, HGK/MAP4K4, HIPK1, HIPK2, HIPK3, HIPK4, HPK1/MAP4K1, IGF1R, IKKa/CHUK, IKKb/IKBKB, IKKe/IKBKE, IR, IRAK1, IRAK4, IRR/INSRR, ITK, JAK1, JAK2, JAK3, JNK1, JNK2, JNK3, KDRNEGFR2, KHS/MAP4K5, LATS1, LATS2, LCK, LCK2/ICK, LKB1, LIMK1, LOK/STK10, LRRK2, LYN, LYNB, MAPKAPK2, MAPKAPK3, MAPKAPK5/PRAK, MARK1, MARK2/PAR-1Ba, MARK3, MARK4, MEK1, MEK2, MEKK1, MEKK2, MEKK3, MELK, MINK/MINK1, MKK4, MKK6, MLCK/MYLK, MLCK2/MYLK2, MLK1/MAP3K9, MLK2/MAP3K10, MLK3/MAP3K11, MNK1, MNK2, MRCKa/, CDC42BPA, MRCKb/, CDC42BPB, MSK1/RPS6KA5, MSK2/RPS6KA4, MSSK1/STK23, MST1/STK4, MST2/STK3, MST3/STK24, MST4, mTOR/FRAP1, MUSK, MYLK3, MYO3b, NEK1, NEK2, NEK3, NEK4, NEK6, NEK7, NEK9, NEK11, NIK/MAP3K14, NLK, OSR1/OXSR1, P38a/MAPK14, P38b/MAPK11, P38d/MAPK13, P38g/MAPK12, P70S6K/RPS6KB1, p70S6Kb/, RPS6KB2, PAK1, PAK2, PAK3, PAK4, PAK5, PAK6, PASK, PBK/TOPK, PDGFRa, PDGFRb, PDK1/PDPK1, PDK1/PDHK1, PDK2/PDHK2, PDK3/PDHK3, PDK4/PDHK4, PHKg1, PHKg2, PI3Ka, (p110a/p85a), PI3Kb, (p110b/p85a), PI3Kd, (p110d/p85a), PI3Kg(p120g), PIM1, PIM2, PIM3, PKA, PKAcb, PKAcg, PKCa, PKCb1, PKCb2, PKCd, PKCepsilon, PKCeta, PKCg, PKCiota, PKCmu/PRKD1, PKCnu/PRKD3, PKCtheta, PKCzeta, PKD2/PRKD2, PKG1a, PKG1b, PKG2/PRKG2, PKN1/PRK1, PKN2/PRK2, PKN3/PRK3, PLK1, PLK2, PLK3, PLK4/SAK, PRKX, PYK2, RAF1, RET, RIPK2, RIPK3, RIPK5, ROCK1, ROCK2, RON/MST1R, ROS/ROS1, RSK1, RSK2, RSK3, RSK4, SGK1, SGK2, SGK3/SGKL, SIK1, SIK2, SLK/STK2, SNARK/NUAK2, SRMS, SSTK/TSSK6, STK16, STK22D/TSSK1, STK25/YSK1, STK32b/YANK2, STK32c/YANK3, STK33, STK38/NDR1, STK38L/NDR2, STK39/STLK3, SRPK1, SRPK2, SYK, TAK1, TAOK1, TAOK2/TAO1, TAOK3/JIK, TBK1, TEC, TESK1, TGFBR2, TIE2/TEK, TLK1, TLK2, TNIK, TNK1, TRKA, TRKB, TRKC, TRPM7/CHAK1, TSSK2, TSSK3/STK22C, TTBK1, TTBK2, TTK, TXK, TYK1/LTK, TYK2, TYRO3/SKY, ULK1, ULK2, ULK3, VRK1, VRK2, WEE1, WNK1, WNK2, WNK3, YES/YES1, ZAK/MLTK, ZAP70, ZIPK/DAPK3, KINASE, MUTANTS, ABL1(E255K), ABL1(F3171), ABL1(G250E), ABL1(H396P), ABL1(M351T), ABL1(Q252H), ABL1(T3151), ABL1(Y253F), ALK (C1156Y), ALK(L1196M), ALK (F1174L), ALK (R1275Q), BRAF(V599E), BTK(E41K), CHK2(1157T), c-Kit(A829P), c-KIT(D816H), c-KIT(D816V), c-Kit(D820E), c-Kit(N822K), C-Kit (T6701), c-Kit(V559D), c-Kit(V559DN654A), c-Kit(V559D/T6701), C-Kit (V560G), c-KIT(V654A), C-MET(D1228H), C-MET(D1228N), C-MET(F12001), c-MET(M1250T), C-MET(Y1230A), C-MET(Y1230C), C-MET(Y1230D), C-MET(Y1230H), c-Src(T341M), EGFR(G719C), EGFR(G719S), EGFR(L858R), EGFR(L861Q), EGFR(T790M), EGFR, (L858R,T790M), EGFR(d746-750/T790M), EGFR(d746-750), EGFR(d747-749/A750P), EGFR(d747-752/P753S), EGFR(d752-759), FGFR1(V561M), FGFR2(N549H), FGFR3(G697C), FGFR3(K650E), FGFR3(K650M), FGFR4(N535K), FGFR4(V550E), FGFR4(V550L), FLT3(D835Y), FLT3(ITD), JAK2 (V617F), LRRK2 (G2019S), LRRK2 (12020T), LRRK2 (R1441C), p38a(T106M), PDGFRa(D842V), PDGFRa(T6741), PDGFRa(V561D), RET(E762Q), RET(G691S), RET(M918T), RET(R749T), RET(R813Q), RET(V804L), RET(V804M), RET(Y791F), TIF2(R849W), TIF2(Y897S), and TIF2(Y1108F).

In another aspect of the invention, the subject compounds may be administered in combination with one or more targeted anti-cancer agents that modulate non-kinase biological targets, pathway, or processes. Such targets pathways, or processes include but not limited to heat shock proteins (e.g. HSP90), poly-ADP (adenosine diphosphate)-ribose polymerase (PARP), hypoxia-inducible factors (HIF), proteasome, Wnt/Hedgehog/Notch signaling proteins, TNF-alpha, matrix metalloproteinase, farnesyl transferase, apoptosis pathway (e.g Bcl-xL, Bcl-2, Bcl-w), histone deacetylases (HDAC), histone acetyltransferases (HAT), and methyltransferase (e.g histone lysine methyltransferases, histone arginine methyltransferase, DNA methyltransferase, etc), and other immunotherapies (e.g anti-PD1, anti-PDL1, anti-CTLA4, CAR-T, IDO, A2A antagonist etc).

In another aspect of the invention, the compounds of the invention are administered in combination with one or more of other anti-cancer agents that include, but are not limited to, gene therapy, RNAi cancer therapy, chemoprotective agents (e.g., amfostine, mesna, and dexrazoxane), antibody conjugate (e.g brentuximab vedotin, ibritumomab tioxetan), cancer immunotherapy such as Interleukin-2, cancer vaccines (e.g., sipuleucel-T) or monoclonal antibodies (e.g., Bevacizumab, Alemtuzumab, Rituximab, Trastuzumab, etc).

In another aspect of the invention, the subject compounds are administered in combination with radiation therapy or surgeries. Radiation is commonly delivered internally (implantation of radioactive material near cancer site) or externally from a machine that employs photon (x-ray or gamma-ray) or particle radiation. Where the combination therapy further comprises radiation treatment, the radiation treatment may be conducted at any suitable time so long as a beneficial effect from the co-action of the combination of the therapeutic agents and radiation treatment is achieved. For example, in appropriate cases, the beneficial effect is still achieved when the radiation treatment is temporally removed from the administration of the therapeutic agents, perhaps by days or even weeks.

In certain embodiments, the compounds of the invention are administered in combination with one or more of radiation therapy, surgery, or anti-cancer agents that include, but are not limited to, DNA damaging agents, anti-metabolites, topoisomerase inhibitors, anti-microtubule agents, kinase inhibitors, epigenetic agents, HSP90 inhibitors, PARP inhibitors, and antibodies targeting VEGF, HER2, EGFR, CD50, CD20, CD30, CD33, etc.

In certain embodiments, the compounds of the invention are administered in combination with one or more of abarelix, abiraterone acetate, aldesleukin, alemtuzumab, altretamine, anastrozole, asparaginase, bendamustine, bevacizumab, bexarotene, bicalutamide, bleomycin, bortezombi, brentuximab vedotin, busulfan, capecitabine, carboplatin, carmustine, cetuximab, chlorambucil, cisplatin, cladribine, clofarabine, clomifene, crizotinib, cyclophosphamide, dasatinib, daunorubicin liposomal, decitabine, degarelix, denileukin diftitox, denileukin diftitox, denosumab, docetaxel, doxorubicin, doxorubicin liposomal, epirubicin, eribulin mesylate, erlotinib, estramustine, etoposide phosphate, everolimus, exemestane, fludarabine, fluorouracil, fotemustine, fulvestrant, gefitinib, gemcitabine, gemtuzumab ozogamicin, goserelin acetate, histrelin acetate, hydroxyurea, ibritumomab tiuxetan, idarubicin, ifosfamide, imatinib mesylate, interferon alfa 2a, ipilimumab, ixabepilone, lapatinib ditosylate, lenalidomide, letrozole, leucovorin, leuprolide acetate, levamisole, lomustine, mechlorethamine, melphalan, methotrexate, mitomycin C, mitoxantrone, nelarabine, nilotinib, oxaliplatin, paclitaxel, paclitaxel protein-bound particle, pamidronate, panitumumab, pegaspargase, peginterferon alfa-2b, pemetrexed disodium, pentostatin, raloxifene, rituximab, sorafenib, streptozocin, sunitinib maleate, tamoxifen, temsirolimus, teniposide, thalidomide, toremifene, tositumomab, trastuzumab, tretinoin, uramustine, vandetanib, vemurafenib, vinorelbine, zoledronate, pembrolizumab, nivolumab, atezolizumab, durvalumab, avelumab, as tisagenlecleucel, axicabtagene ciloleucel, radiation therapy, or surgery.

The invention further provides methods for the prevention or treatment of a neoplastic disease or autoimmune disease. In one embodiment, the invention relates to a method of treating a neoplastic disease or autoimmune disease, in a subject in need of treatment comprising administering to said subject a therapeutically effective amount of a compound of the invention. In one embodiment, the invention further provides for the use of a compound of the invention in the manufacture of a medicament for halting or decreasing a neoplastic disease or autoimmune disease.

In certain embodiments, the neoplastic disease is a lung cancer, head and neck cancer, central nervous system cancer, prostate cancer, testicular cancer, colorectal cancer, pancreatic cancer, liver cancer, stomach cancer, biliary tract cancer, esophageal cancer, gastrointestinal stromal tumor, breast cancer, cervical cancer, ovarian cancer, uterine cancer, leukemia, lymphomas, multiple myeloma, melanoma, basal cell carcinoma, squamous cell carcinoma, bladder cancer, renal cancer, sarcoma, mesothelioma, thymoma, myelodysplastic syndrome, or myeloproliferative disease.

The autoimmune diseases that can be affected using compounds and compositions according to the invention include, but are not limited to allergy, Alzheimer's disease, acute disseminated encephalomyelitis, Addison's disease, ankylosing spondylitis, antiphospholipid antibody syndrome, asthma, atherosclerosis, autoimmune hemolytic anemia, autoimmune hemolytic and thrombocytopenic states, autoimmune hepatitis, autoimmune inner ear disease, bullous pemphigoid, coeliac disease, chagas disease, chronic obstructive pulmonary disease, chronic Idiopathic thrombocytopenic purpura (ITP), churg-strauss syndrome, Crohn's disease, dermatomyositis, diabetes mellitus type 1, endometriosis, Goodpasture's syndrome (and associated glomerulonephritis and pulmonary hemorrhage), graves' disease, guillain-barré syndrome, hashimoto's disease, hidradenitis suppurativa, idiopathic thrombocytopenic purpura, interstitial cystitis, irritable bowel syndrome, lupus erythematosus, morphea, multiple sclerosis, myasthenia gravis, narcolepsy, neuromyotonia, Parkinson's disease, pemphigus vulgaris, pernicious anaemia, polymyositis, primary biliary cirrhosis, psoriasis, psoriatic arthritis, rheumatoid arthritis, schizophrenia, septic shock, scleroderma, Sjogren's disease, systemic lupus erythematosus (and associated glomerulonephritis), temporal arteritis, tissue graft rejection and hyperacute rejection of transplanted organs, vasculitis (ANCA-associated and other vasculitides), vitiligo, and wegener's granulomatosis.

It should be understood that the invention is not limited to the particular embodiments shown and described herein, but that various changes and modifications may be made without departing from the spirit and scope of the invention as defined by the claims.

The compounds according to the present invention may be synthesized according to a variety of schemes. Necessary starting materials may be obtained by standard procedures of organic chemistry. The compounds and processes of the present invention will be better understood in connection using the following representative synthetic schemes and examples, which are intended as an illustration only and not limiting of the scope of the invention. Various changes and modifications to the disclosed embodiments will be apparent to those skilled in the art and such changes and modifications including, without limitation, those relating to the chemical structures, substituents, derivatives, and/or methods of the invention may be made without departing from the spirit of the invention and the scope of the appended claims.

A typical approach to synthesize the intermediate

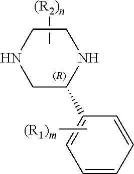

is described in Scheme 1-1 below.

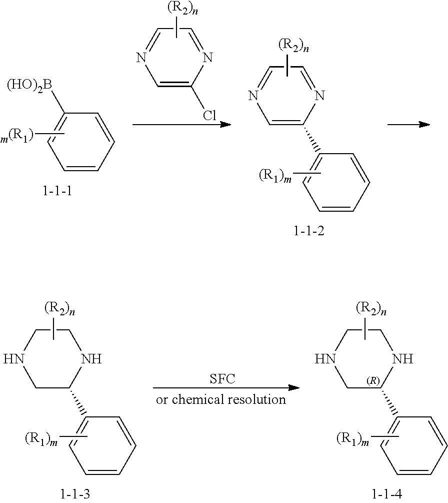

In Scheme 1-1, the starting material 1-1-1 can be prepared by conventional procedures using appropriate compounds and reagent. The starting material 1-1-1 is converted to intermediate 1-1-2 through a Suzuki coupling reaction, which can be hydrogenated to afford 1-1-3 readily. Finally, the enantiomer 1-1-4 can be separated from 1-1-3 (racemate) via chiral resolution.

Also, the target compounds can be synthesized by alternative methods but not limited to the above procedures.

A typical approach to synthesize the intermediate

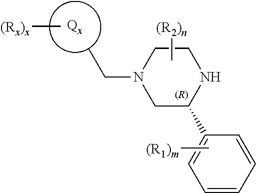

is described in Scheme 1-2 below.

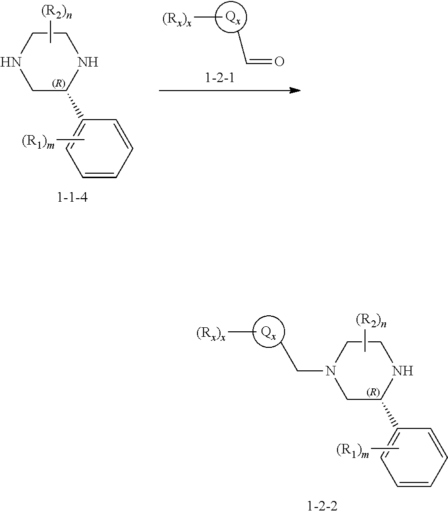

In Scheme 1-2, the starting material 1-1-4 can be prepared by conventional procedures using appropriate compounds and reagent. The starting material 1-1-4 can react with 1-2-1 to afford 1-2-2 via reductive amination.

Also, the target compounds can be synthesized by alternative methods but not limited to the above procedures.

A typical approach to synthesize the intermediate

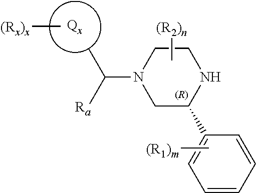

is described in Scheme 1-3 below.

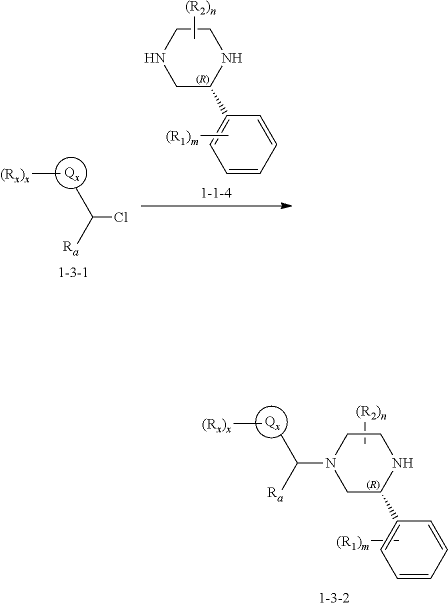

In Scheme 1-3, the starting material 1-3-1 can be prepared by conventional procedures using appropriate compounds and reagent. The starting material 1-3-1 can react with 1-1-4 to afford 1-3-2 readily.

Also, the target compounds can be synthesized by alternative methods but not limited to the above procedures.

A typical approach to synthesize the intermediate

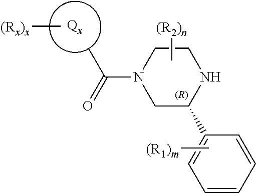

is described in Scheme 1-4 below.

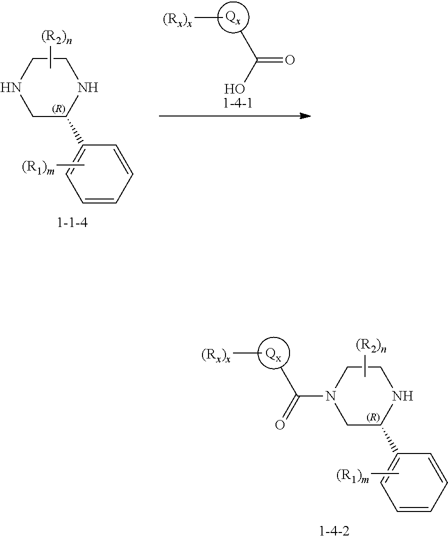

In Scheme 1-4, the starting material 1-1-4 can be prepared by conventional procedures using appropriate compounds and reagent. The starting material 1-1-4 can react with 1-4-1 to give 1-4-2 under a suitable condition.

Also, the target compounds can be synthesized by alternative methods but not limited to the above procedures.

A typical approach to synthesize the intermediate

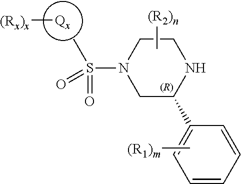

is described in Scheme

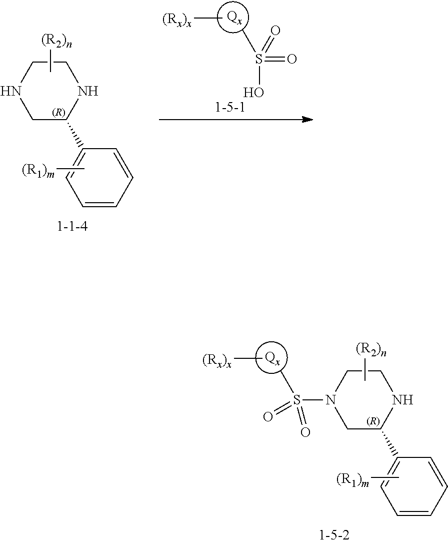

In Scheme 1-5, the starting material 1-1-4 can be prepared by conventional procedures using appropriate compounds and reagent. The starting material 1-1-4 can react with sulfonyl chloride 1-5-1 to give the target compound 1-5-2 under a suitable condition.

Also, the target compounds can be synthesized by alternative methods but not limited to the above procedures.

A typical approach to synthesize the intermediate

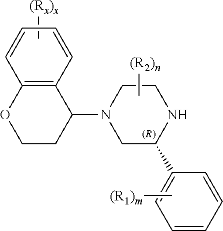

is described in Scheme 1-6 below.

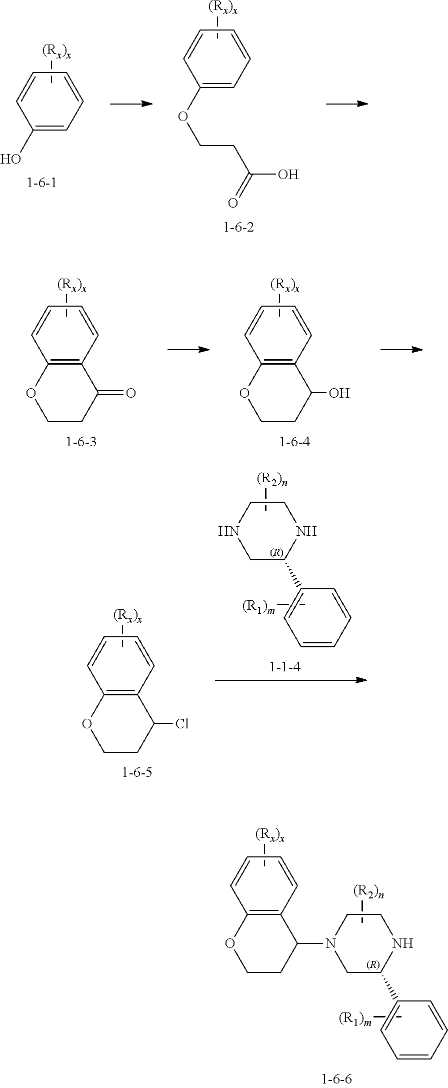

In Scheme 1-6, the starting material 1-6-1 can be prepared by conventional procedures using appropriate compounds and reagent. The starting material 1-6-1 can be alkylated to give 1-6-2 readily, which is converted to intermediate 1-6-3 through a Friedel-Crafts acylation reaction. Next, the intermediate 1-6-3 is reduced to 1-6-4 by a suitable reagent, which can be transform to chlorides 1-6-5. Finally, 1-6-5 can react with 1-1-4 to yield the target compound 1-6-6.

Also, the target compounds can be synthesized by alternative methods but not limited to the above procedures.

A typical approach to synthesize the intermediate

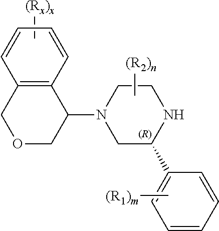

is described in Scheme 1-7 below.

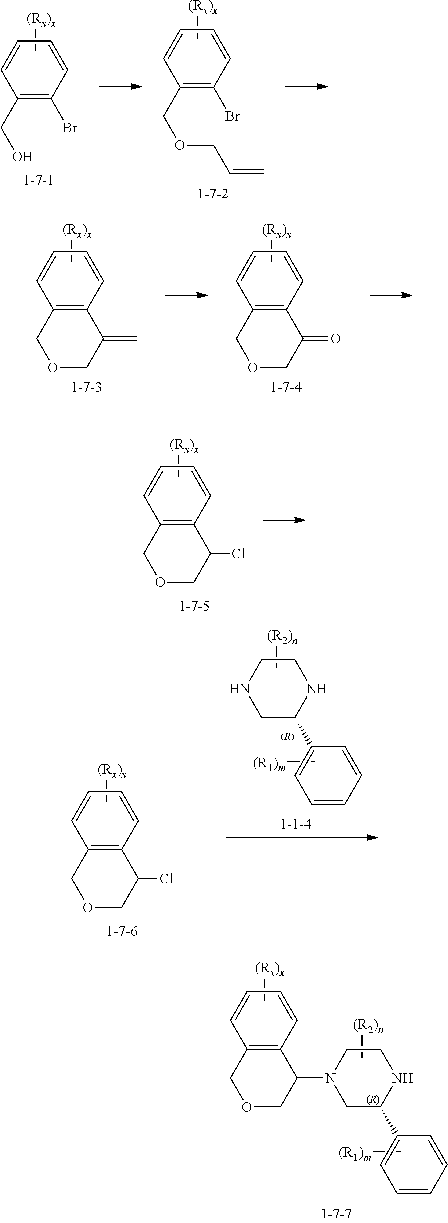

In Scheme 1-7, the starting material 1-7-1 can be prepared by conventional procedures using appropriate compounds and reagent. The starting material 1-7-1 can be alkylated to give 1-7-2 readily, which is converted to intermediate 1-7-3 through Heck coupling reaction. Next, the intermediate 1-7-3 is converted to 1-7-4 by a suitable reagent, which can be reduced to 1-7-5. Finally, 1-7-5 can be transform to chlorides 1-7-6, which can react with 1-1-4 to yield the target compound 1-7-7.

Also, the target compounds can be synthesized by alternative methods but not limited to the above procedures.

A typical approach to synthesize the intermediate

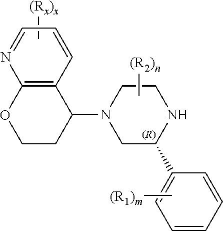

is described in Scheme 1-8 below.

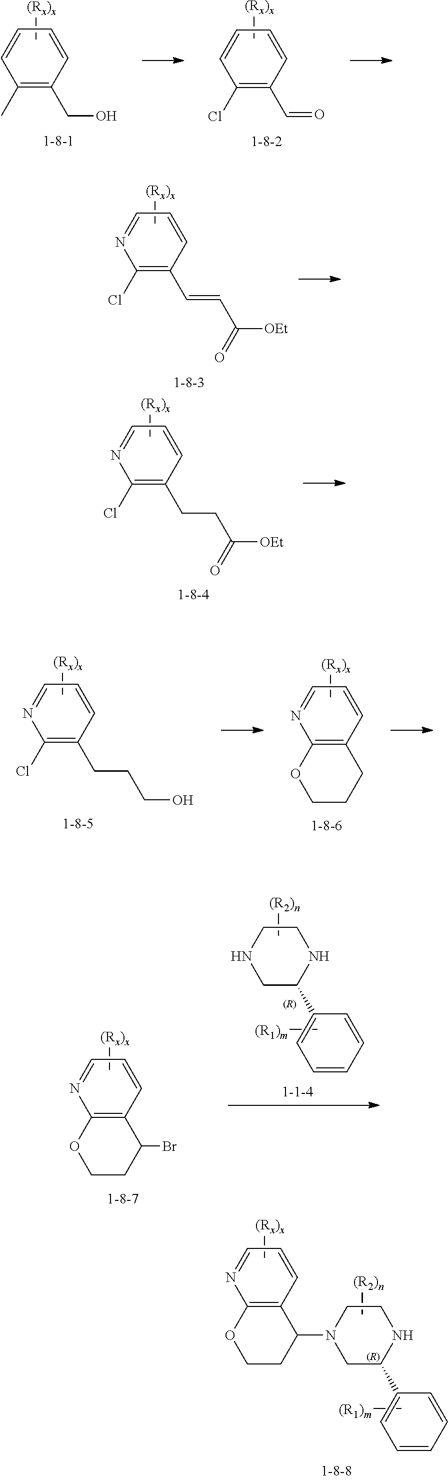

In Scheme 1-8, the starting material 1-8-1 can be prepared by conventional procedures using appropriate compounds and reagent. The starting material 1-8-1 can be oxidized to give 1-8-2 readily, which is converted to intermediate 1-8-3 through Wittig reaction. After that, the intermediate 1-8-3 is hydrogenated to 1-8-4 under a suitable condition, which can be reduced to 1-8-5. Next, the intermediate 1-8-5 can be converted to 1-8-6 via a intramolecular cyclization. Finally, 1-8-6 can be transform to bromides 1-8-7, which is react with 1-1-4 to yield the target compound 1-8-8.

Also, the target compounds can be synthesized by alternative methods but not limited to the above procedures.

A typical approach to synthesize the intermediate

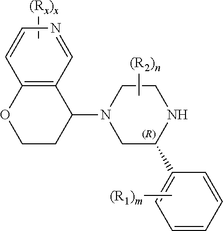

is described in Scheme 1-9 below.

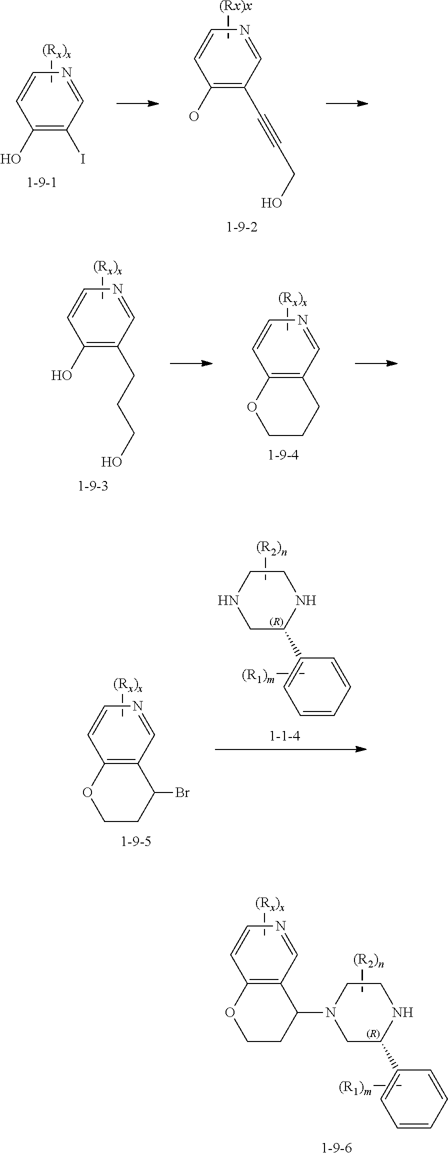

In Scheme 1-9, the starting material 1-9-1 can be prepared by conventional procedures using appropriate compounds and reagent. The starting material 1-9-1 can be converted to intermediate 1-9-2 through Sonogashira coupling reaction. After that, the intermediate 1-9-2 is hydrogenated to 1-9-3 under a suitable condition, which can be converted to 1-9-4 via a intramolecular cyclization. Finally, 1-9-4 can be transform to bromides 1-9-5, which is react with 1-1-4 to yield the target compound 1-9-6.

Also, the target compounds can be synthesized by alternative methods but not limited to the above procedures.

A typical approach to synthesize the intermediate

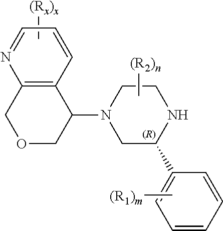

is described in Scheme 1-10 below.

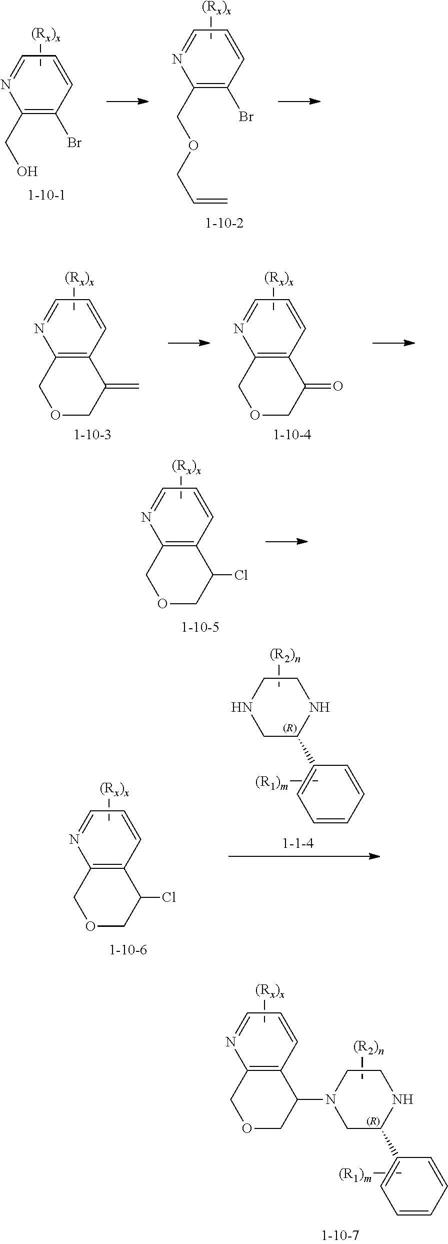

In Scheme 1-10, the starting material 1-10-1 can be prepared by conventional procedures using appropriate compounds and reagent. The starting material 1-10-1 can be alkylated to give 1-10-2 readily, which is converted to intermediate 1-10-3 through Heck reaction. After that, the intermediate 1-10-3 is oxidized to 1-10-4 under a suitable condition, which can be reduced to 1-10-5. Finally, 1-10-5 can be transform to chlorides 1-10-6, which is react with 1-1-4 to yield the target compound 1-10-7.

Also, the target compounds can be synthesized by alternative methods but not limited to the above procedures.

A typical approach to synthesize the intermediate

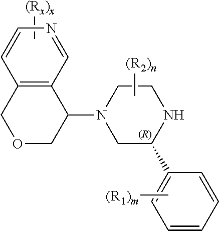

is described in Scheme 1-11 below.

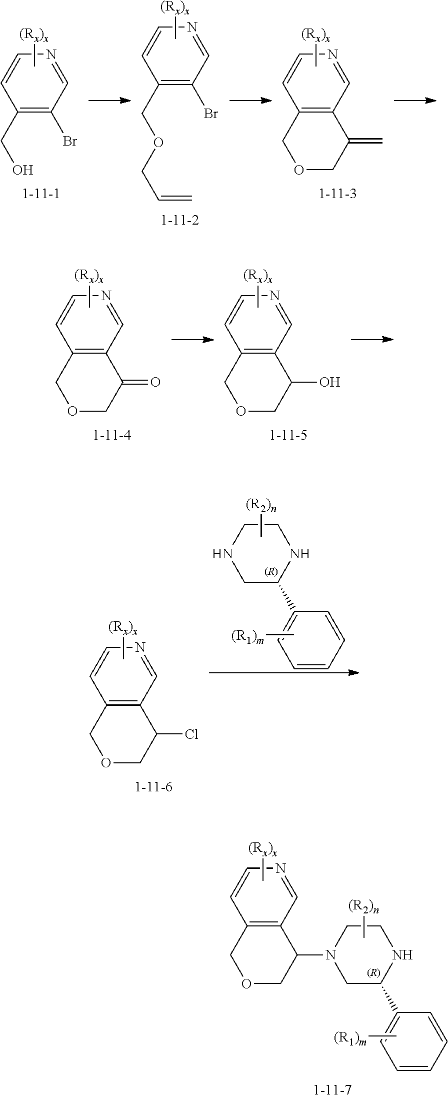

In Scheme 1-11, the starting material 1-11-1 can be prepared by conventional procedures using appropriate compounds and reagent. The starting material 1-11-1 can be alkylated to give 1-11-2 readily, which is converted to intermediate 1-11-3 through Heck reaction. After that, the intermediate 1-11-3 is oxidized to 1-11-4 under a suitable condition, which can be reduced to 1-11-5. Finally, 1-11-5 can be transform to chlorides 1-11-6, which is react with 1-1-4 to yield the target compound 1-11-7.

Also, the target compounds can be synthesized by alternative methods but not limited to the above procedures.

A typical approach to synthesize the intermediate

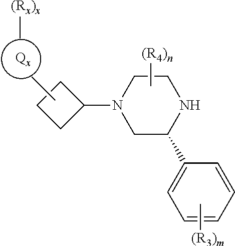

is described in Scheme 1-11-A below.

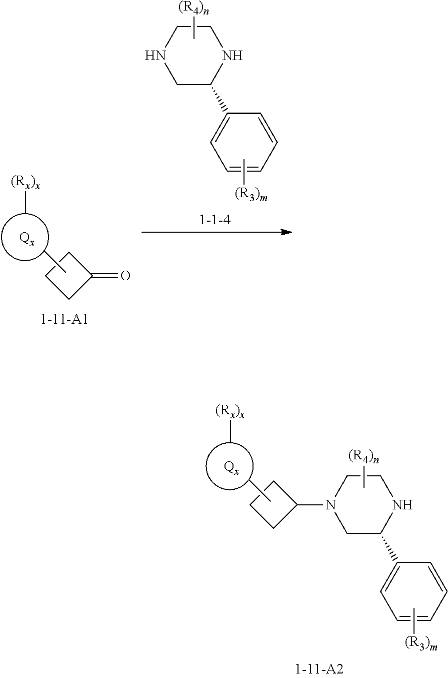

In Scheme 1-11-A, the starting material 1-11-A1 can be prepared by conventional procedures using appropriate compounds and reagent. The starting material 1-11-A1 can react with 1-1-4 to yield the target compound 1-11-A2 via reductive amination reaction.

Also, the target compounds can be synthesized by alternative methods but not limited to the above procedures.

A typical approach to synthesize the intermediate

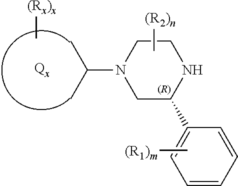

wherein Q is cycloalkyl, spirocycloalkyl, fused-carbocyclic, bridged-carbocyclic, heterocycloalkyl, spiro-heterocyclic, fused-heterocyclic, or bridged-heterocyclic is described in Scheme 1-12 below.

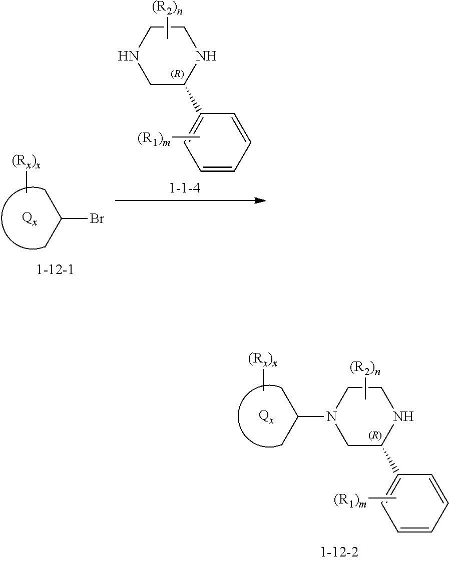

In Scheme 1-12, the starting material 1-12-1 can be prepared by conventional procedures using appropriate compounds and reagent. The starting material 1-12-1 can react with 1-1-4 to yield the target compound 1-12-2 via SN₂ substitution reaction.

Also, the target compounds can be synthesized by alternative methods but not limited to the above procedures.

A typical approach to synthesize the intermediate

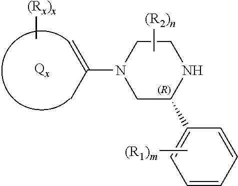

wherein Q is aryl, or heteroaryl is described in Scheme 1-13 below.

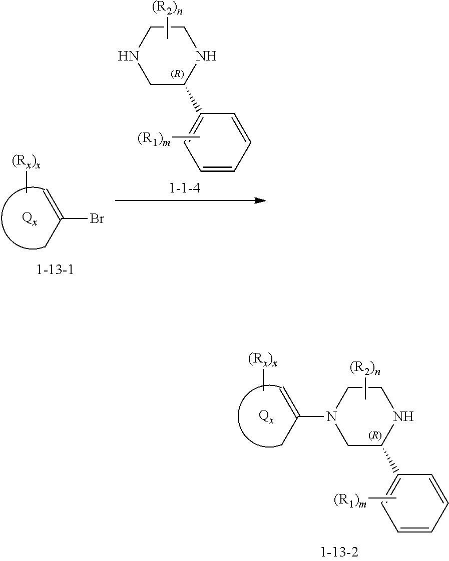

In Scheme 1-13, the starting material 1-13-1 can be prepared by conventional procedures using appropriate compounds and reagent. The starting material 1-13-1 can react with 1-1-4 to yield the target compound 1-13-2 via Buchwald coupling reaction.

Also, the target compounds can be synthesized by alternative methods but not limited to the above procedures.

A typical approach to synthesize the intermediate

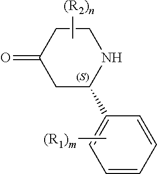

is described in Scheme 1-14 below.

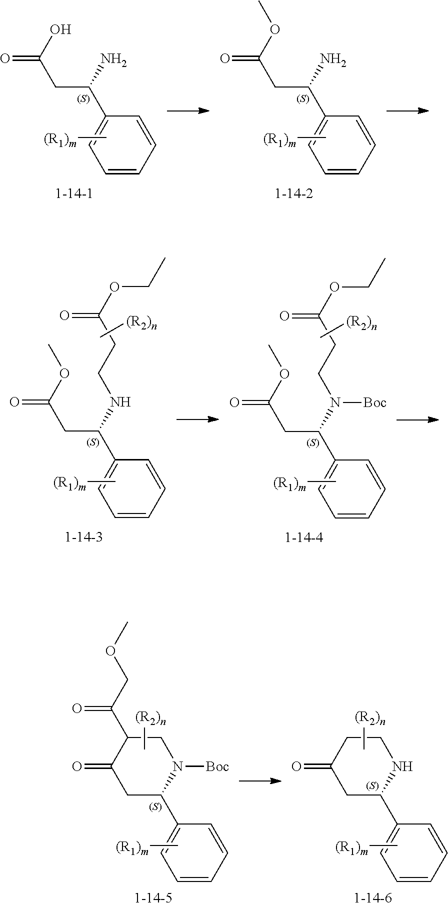

In Scheme 1-14, the starting material 1-14-1 can be prepared by conventional procedures using appropriate compounds and reagent. The starting material 1-14-1 can be converted to 1-14-2 readily, which is alkylated to give intermediate 1-14-3. After that, the intermediate 1-14-3 can be protected to afford 1-14-4, which is transform to 1-14-5 through an intramolecular reaction. Finally, the intermediate 1-14-5 is decarboxylated to yield the target compound 1-14-6.

Also, the target compounds can be synthesized by alternative methods but not limited to the above procedures.

A typical approach to synthesize the intermediate

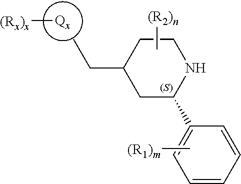

is described in Scheme 1-15 below.

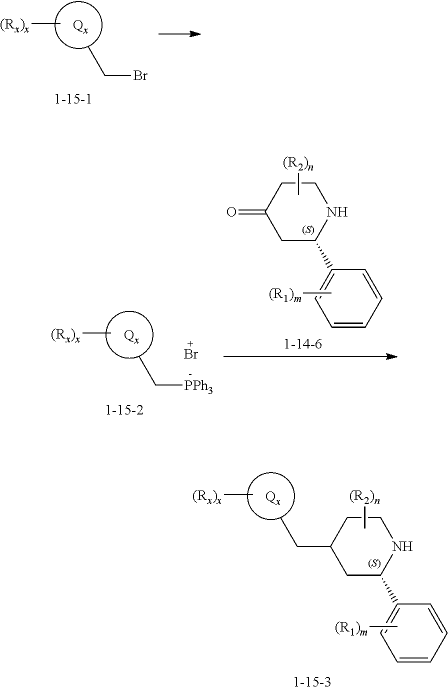

In Scheme 1-15, the starting material 1-15-1 can be prepared by conventional procedures using appropriate compounds and reagent. The starting material 1-15-1 can be converted to phosphonium ylide salts 1-15-2 readily, which can react with 1-14-6 to give the target compound via Wittig reaction.

Also, the target compounds can be synthesized by alternative methods but not limited to the above procedures.

A typical approach to synthesize the intermediate

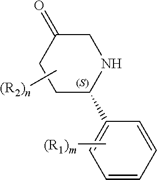

is described in Scheme 1-16 below.

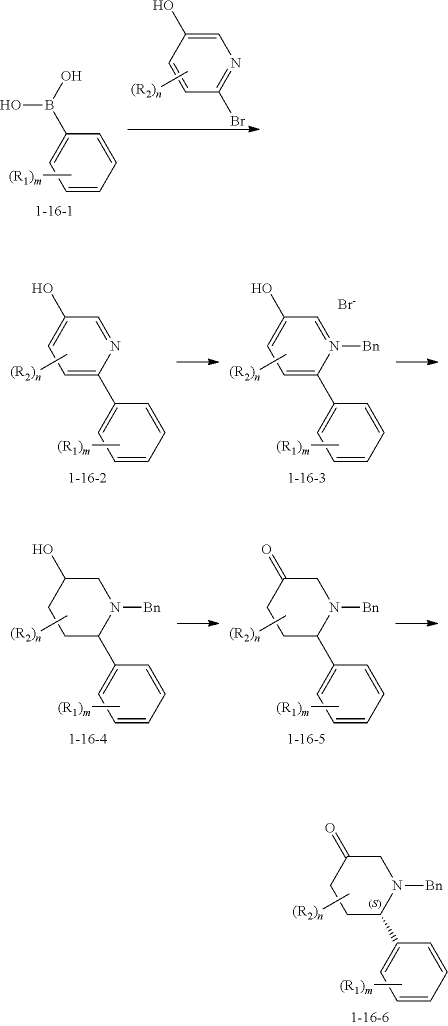

In Scheme 1-16, the starting material 1-16-1 can be prepared by conventional procedures using appropriate compounds and reagent. The starting material 1-16-1 can be converted to intermediate 1-16-2 through Suzuki coupling reaction. After that, the intermediate 1-16-2 is alkylated to give quaternary ammonium salt 1-16-3, which can be reduced to 1-16-4 readily. Finally, 1-16-4 can be oxidated to 1-16-5, which is deprotected to yield the target compound 1-16-6.

Also, the target compounds can be synthesized by alternative methods but not limited to the above procedures.

A typical approach to synthesize the intermediate

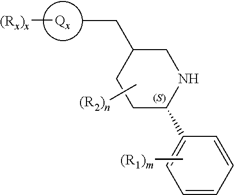

is described in Scheme 1-17 below.

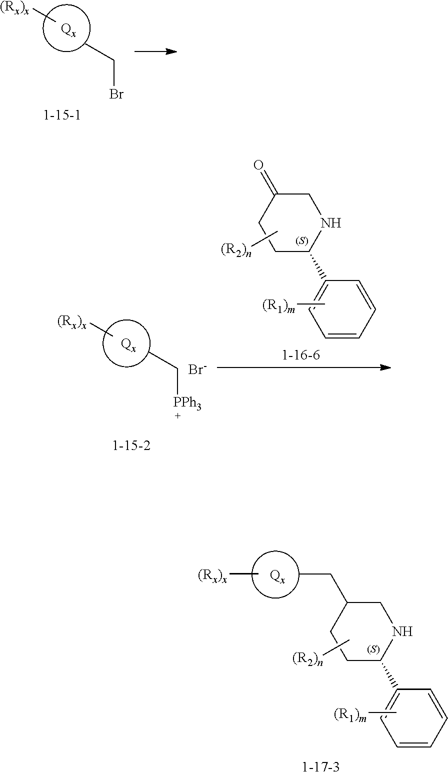

In Scheme 1-17, the starting material 1-17-1 can be prepared by conventional procedures using appropriate compounds and reagent. The starting material 1-17-1 can be converted to phosphonium ylide salts 1-17-2 readily, which can react with 1-16-6 to give the target compound 1-17-3 via Wittig reaction.

Also, the target compounds can be synthesized by alternative methods but not limited to the above procedures.

Similarly, the intermediate of

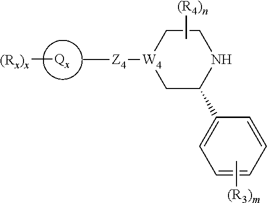

can be prepared by the method similar to the Scheme 1-1 to Scheme 1-17 by using appropriate staring materials, intermediates, and intramolecular cyclization.

Similarly, the intermediate of

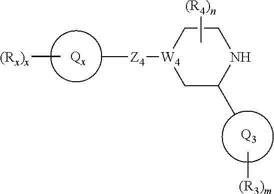

can be prepared by the method similar to the Scheme 1-1 to Scheme 1-17 by using appropriate staring materials, intermediates, and intramolecular cyclization.

A typical approach to synthesize the intermediate

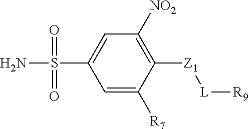

is described in Scheme 2-1 below.

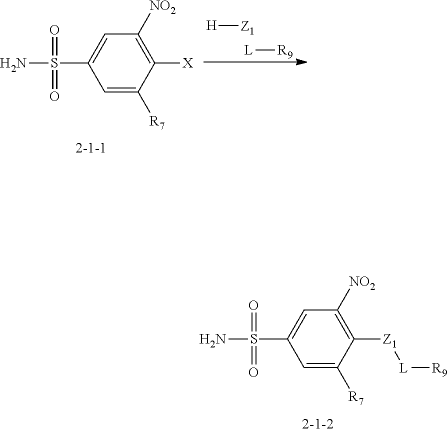

In Scheme 2-1, the starting material 2-1-1 reacts with appropriate alcohol or amine will yield 2-1-2.

Also, the target compounds can be synthesized by alternative methods but not limited to the above procedures.

Similarly, the intermediate of

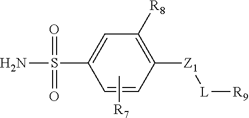

can be prepared by the method similar to Scheme 2-1 by using appropriate staring materials, and intermediates.

A typical approach to synthesize the intermediate

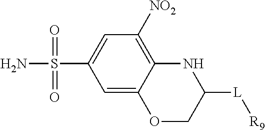

is described in Scheme 2-2A below.

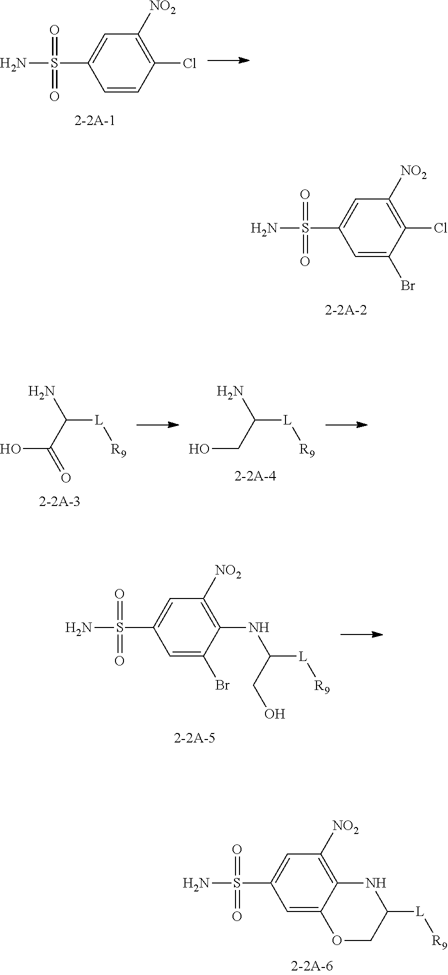

In Scheme 2-2A, the starting material 2-2A-1 can be prepared by conventional procedures using appropriate compounds and reagent. The starting material 2-2A-1 can be brominated to give intermediated 2-2A-2. Next, 2-2A-3 is reduced to give Amino alcohol 2-2A-4, which can be converted to 2-2A-5. Finally, 2-2A-6 can be obtained via an intermolecular cyclization reaction.

Also, the target compounds can be synthesized by alternative methods but not limited to the above procedures.

A typical approach to synthesize the intermediate

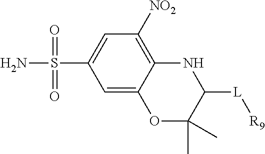

is described in Scheme 2-2B below.

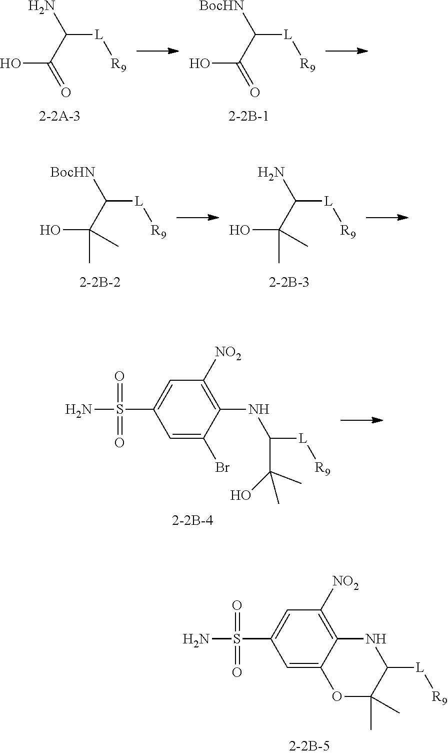

In Scheme 2-2B, the starting material 2-2A-3 can be prepared by conventional procedures using appropriate compounds and reagent. The starting material 2-2A-3 is converted to intermediated 2-2B-1, which can be further converted to 2-2B-2. Next, 2-2B-2 is deprotected to give Amino alcohol 2-2B-3, which can be transform to 2-2B-4. Finally, 2-2B-5 can be obtained via an intermolecular cyclization.

Also, the target compounds can be synthesized by alternative methods but not limited to the above procedures.

A typical approach to synthesize the intermediate

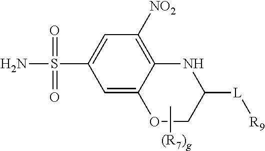

is described in Scheme 2-2C below.

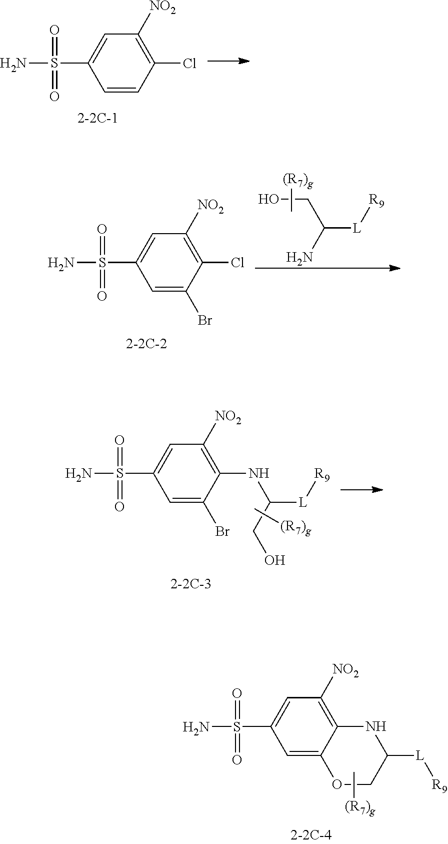

In Scheme 2-2C, the bromination of the commercially available 2-2C-1 results in 2-2C-2, and then the reaction of 2-2C-2 with appropriate amine provides 2-2C-3. Intramolecular cyclization of 2-2C-3 using metal-catalyzed coupling condition such as Buchwald reaction or other coupling reaction known in the literatures give 2-2C-4. Alternatively, 2-2C-4 can be obtained via a three-step sequence of mesylation of the hydroxyl group of 2-2C-3, SN₂ reaction and intramolecular cyclization.

Also, the target compounds can be synthesized by alternative methods but not limited to the above procedures.

A typical approach to synthesize the intermediate

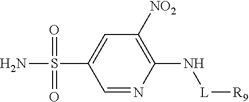

is described in Scheme 2-3 below.

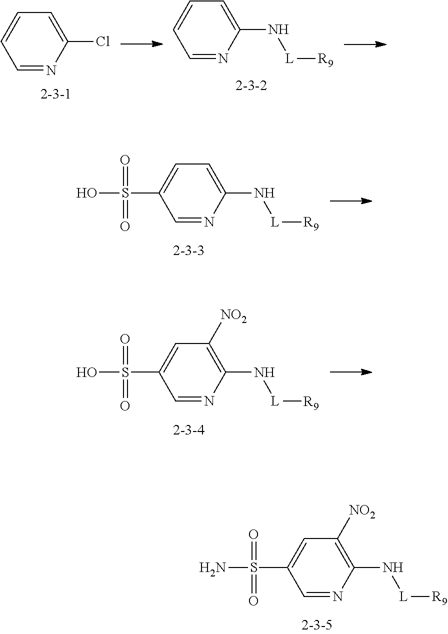

In Scheme 2-3, the starting material 2-3-1 can be prepared by conventional procedures using appropriate compounds and reagents. The starting material 2-3-1 can be converted to 2-3-2 via a SNAr reaction, and then the sulfonylation of 2-3-2 can afford the intermediate 2-3-3. Finally, the intermediate 2-3-3 goes through a two-step sequence of conventional organic reaction to yield the target compounds 2-3-5.

Also, the target compounds can be synthesized by alternative methods but not limited to the above procedures.

A typical approach to synthesize the intermediate

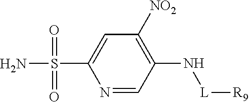

is described in Scheme 2-4 below.

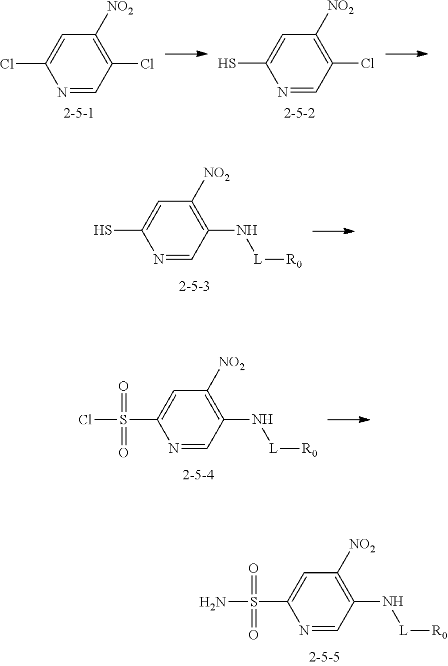

In Scheme 2-4, the starting material 2-4-1 can be prepared by conventional procedures using appropriate compounds and reagents. The starting material 2-4-1 can be converted to 2-4-3 via a two-step sequence of conventional organic reaction. Finally, the intermediate 2-4-3 can be converted to the corresponding sulfonyl chlorides, which can react with ammonia to yield the target compounds 2-4-5.

Also, the target compounds can be synthesized by alternative methods but not limited to the above procedures.

A typical approach to synthesize the intermediate

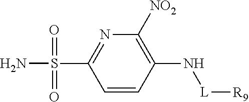

is described in Scheme 2-5 below.

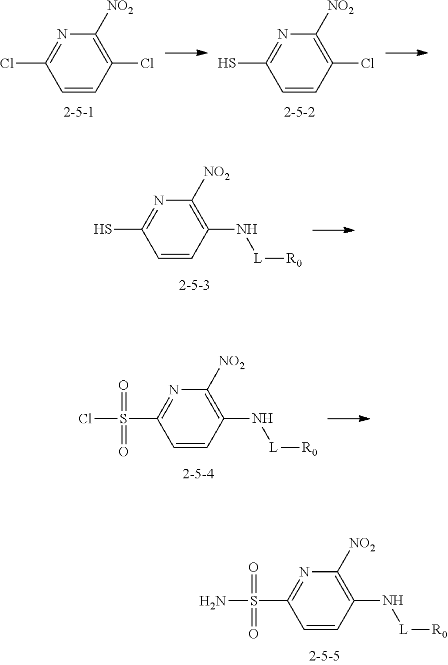

In Scheme 2-5, the starting material 2-5-1 can be prepared by conventional procedures using appropriate compounds and reagents. The starting material 2-5-1 can be converted to 2-5-3 via a two-step sequence conventional organic reaction. Finally, the intermediate 2-5-3 can be converted to the corresponding sulfonyl chlorides 2-5-4, which can react with ammonia to yield the target compounds 2-5-5.

Also, the target compounds can be synthesized by alternative methods but not limited to the above procedures.

A typical approach to synthesize the intermediate

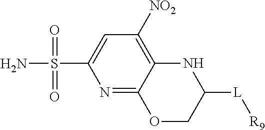

is described in Scheme 2-6 below.

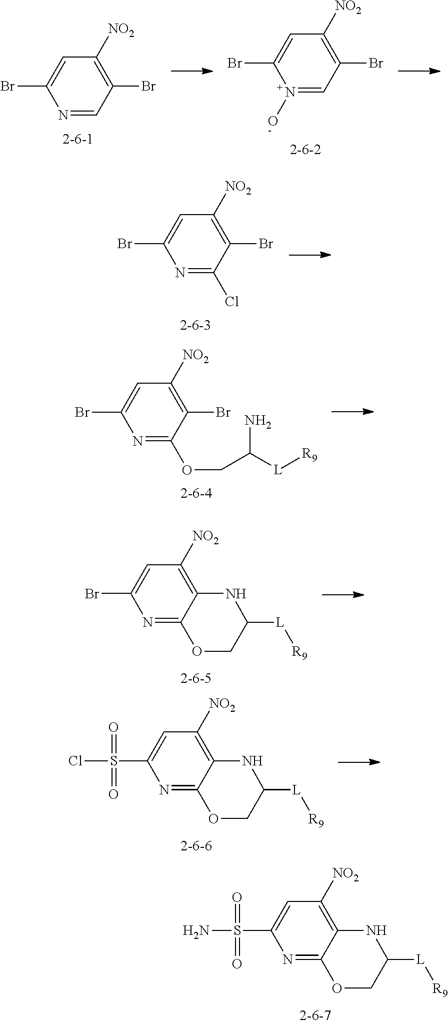

In Scheme 2-6, the starting material 2-6-1 can be prepared by conventional procedures using appropriate compounds and reagents. The starting material 2-6-1 can be oxidized to yield 2-6-2, which can be converted to the intermediate 2-6-3 readily. After that, the intermediate 2-6-3 undergoes a SNAr reaction to give 2-6-4, which is converted to 2-6-5 through an intramolecular cyclization reaction. Finally, the intermediate 2-6-5 can be converted to the corresponding sulfonyl chlorides 2-6-6, which can react with ammonia to yield the target compounds 2-6-7.

Also, the target compounds can be synthesized by alternative methods but not limited to the above procedures.

A typical approach to synthesize the intermediate

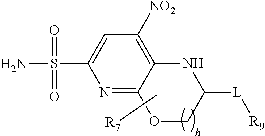

in which h is 0, 1, or 2 is described in Scheme 2-7 below.

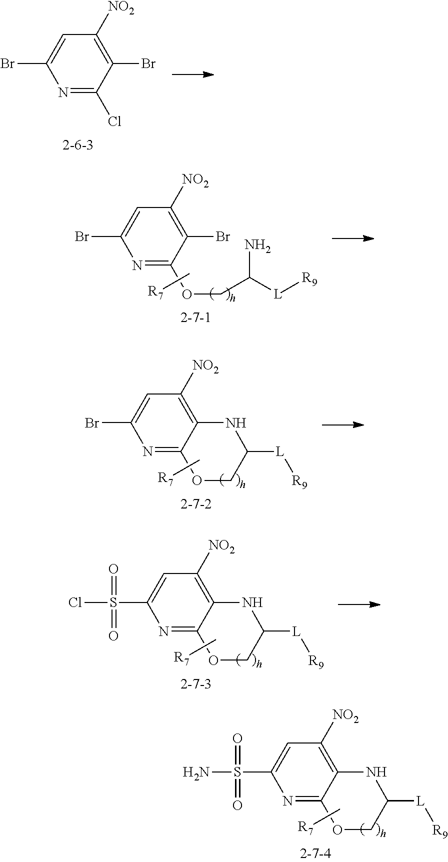

In Scheme 2-7, the starting material 2-6-3 can be prepared by conventional procedures using appropriate compounds and reagents. The starting material 2-6-3 undergoes a SNAr reaction to give 2-7-1, which is converted to 2-7-2 through an intramolecular cyclization reaction. Finally, the intermediate 2-7-2 can be converted to the corresponding sulfonyl chlorides 2-7-3, which can further react with ammonia to yield the target compounds 2-7-4.

Also, the target compounds can be synthesized by alternative methods but not limited to the above procedures.

A typical approach to synthesize the intermediate

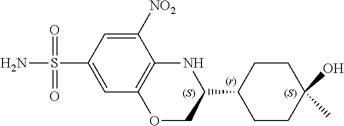

is described in Scheme 2-8 below.

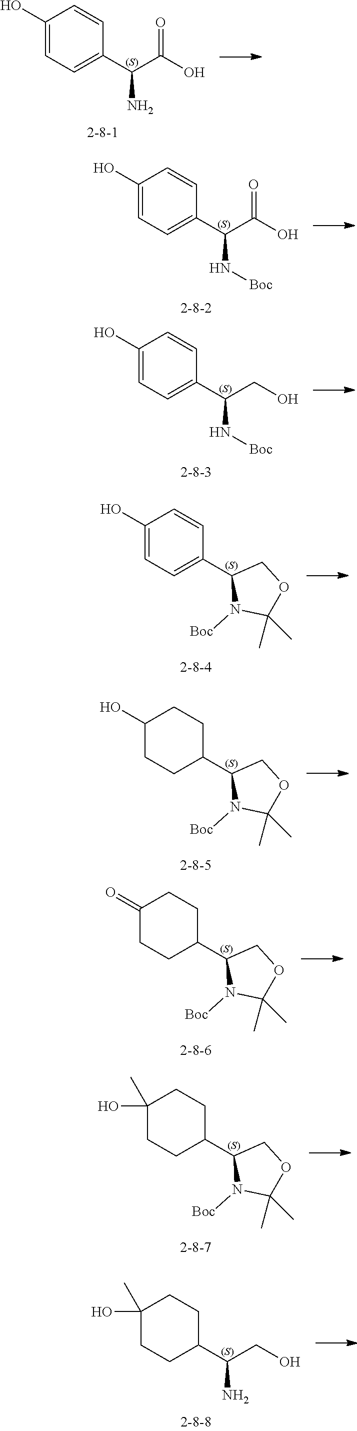

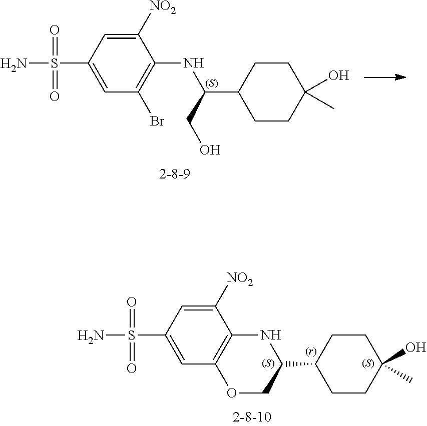

In Scheme 2-8, the starting material 2-8-1 can be prepared by conventional procedures using appropriate compounds and reagents. The starting material 2-8-1 can be converted to 2-8-3 through a two-step sequence of conventional organic reactions. After that, the intermediate 2-8-3 is protected to give 2-8-4, which can be further reduced to yield the intermediate 2-8-5. The intermediate 2-8-5 is oxidized to generate the ketone 2-8-6, which can be converted to 2-8-7 readily. The intermediate 2-8-7 is deprotected to afford 2-8-8, which can be further converted to the intermediate 2-8-9. Finally, 2-8-9 undergoes an intramolecular cyclization to afford the target compounds 2-8-10.

Also, the target compounds can be synthesized by alternative methods but not limited to the above procedures.

A typical approach to synthesize the intermediate

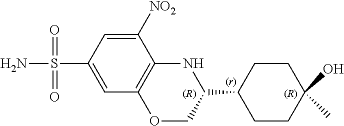

is described in Scheme 2-9 below.

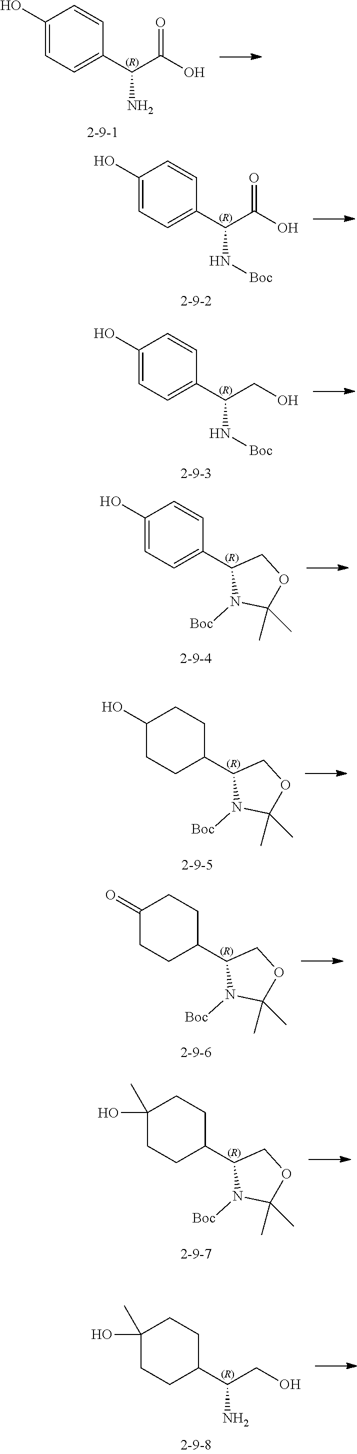

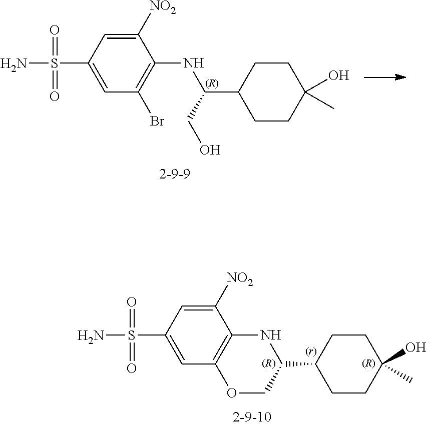

In Scheme 2-9, the starting material 2-9-1 can be prepared by conventional procedures using appropriate compounds and reagents. The starting material 2-9-1 can be converted to 2-9-3 through a two-step sequence of conventional organic reactions. After that, the intermediate 2-9-3 is protected to give 2-9-4, which can be further reduced to yield the intermediate 2-9-5. The intermediate 2-9-5 is oxidized to generate the ketone 2-9-6, which can be converted to 2-9-7 readily. The intermediate 2-9-7 is deprotected to afford 2-9-8, which can be further converted to the intermediate 2-9-9. Finally, 2-9-9 undergoes an intramolecular cyclization to afford the target compounds 2-9-10.

Also, the target compounds can be synthesized by alternative methods but not limited to the above procedures.

A typical approach to synthesize the intermediate

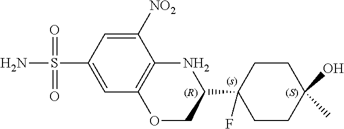

is described in Scheme 2-10 below.

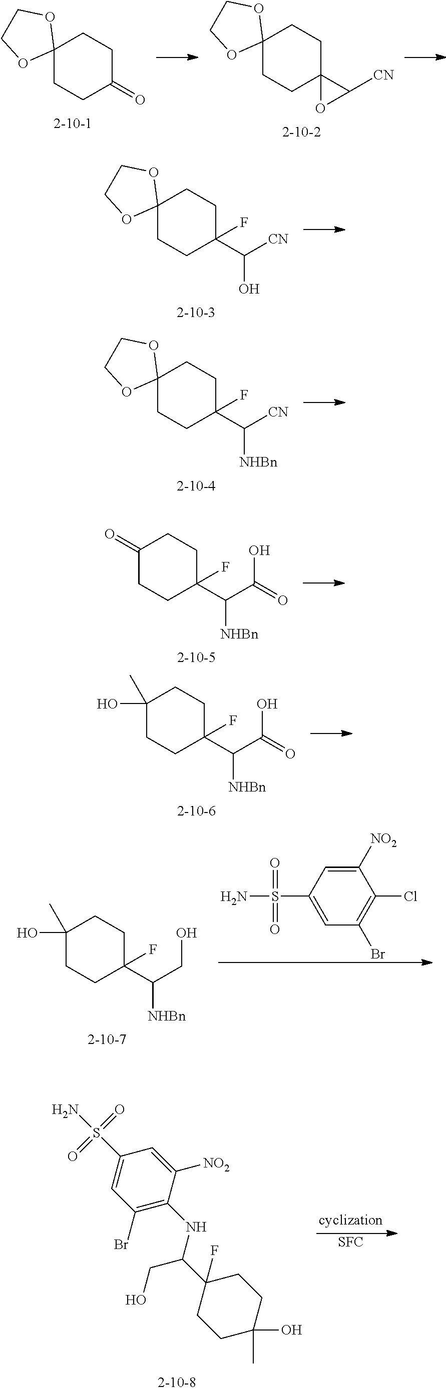

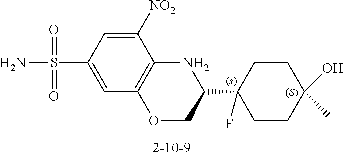

In Scheme 2-10, the starting material 2-10-1 can be prepared by conventional procedures using appropriate compounds and reagents. The starting material 2-10-1 can be converted to 2-10-3 through a two-step sequence of literate reported reactions. After that, the intermediate 2-10-3 can be converted to 2-10-4, which can be further hydrolyzed to yield the intermediate 2-10-5. The intermediate 2-10-5 can be converted to 2-10-6 readily, which is reduced to give 2-10-7. Finally, the intermediate 2-10-7 can be converted to the intermediate 2-10-8, which undergoes an intramolecular cyclization and through a further chiral separation to afford the target compounds 2-10-9.

Also, the target compounds can be synthesized by alternative methods but not limited to the above procedures.

A typical approach to synthesize the intermediate

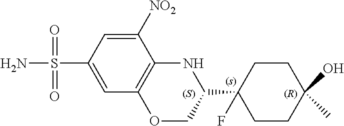

is described in Scheme 2-11 below.

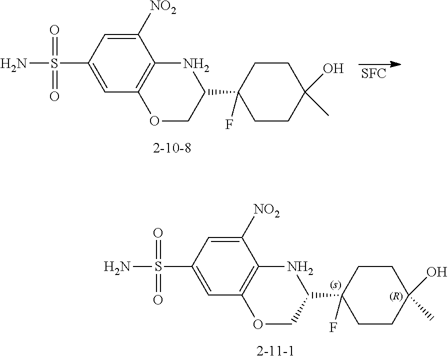

In Scheme 2-11, the starting material 2-10-8 can be prepared by conventional procedures using appropriate compounds and reagents. The starting material 2-10-8 can goes through a chiral separation to afford the target compound 2-11-1.

Also, the target compounds can be synthesized by alternative methods but not limited to the above procedures.

Similarly, the intermediate of

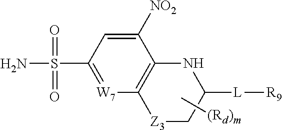

can be prepared by the method similar to the Scheme 2-1 to Scheme 2-11 by using appropriate staring materials, intermediates, and intramolecular cyclization.

Similarly, the intermediate of

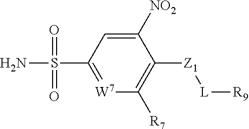

can be prepared by the method similar to the Scheme 2-1 to Scheme 2-11 by using appropriate staring materials, intermediates, and intramolecular cyclization.

Similarly, the intermediate of

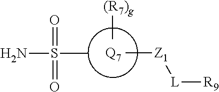

can be prepared by the method similar to the Scheme 2-1 to Scheme 2-11 by using appropriate staring materials, intermediates, and intramolecular cyclization.

A typical approach to synthesize the intermediate

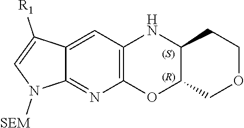

is described in Scheme 3-0-A below.

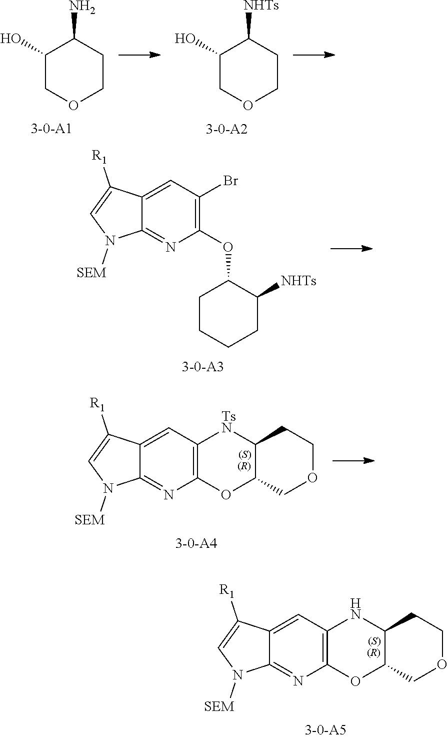

In Scheme 3-0-A, the starting material 3-0-A1 can be prepared by conventional procedures using appropriate compounds and reagents. The starting material 3-0-A1 can be converted to 3-0-A2 readily, which further undergoes a SNAr reaction to give 3-0-A3. Finally, 3-0-A3 can be converted to 3-0-A4 through an intramolecular cyclization reaction, which is deprotected to afford the target compounds 3-0-A5.

Also, the target compounds can be synthesized by alternative methods but not limited to the above procedures.

A typical approach to synthesize the intermediate

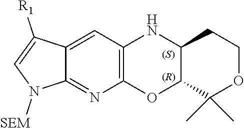

is described in Scheme 3-0-B below.

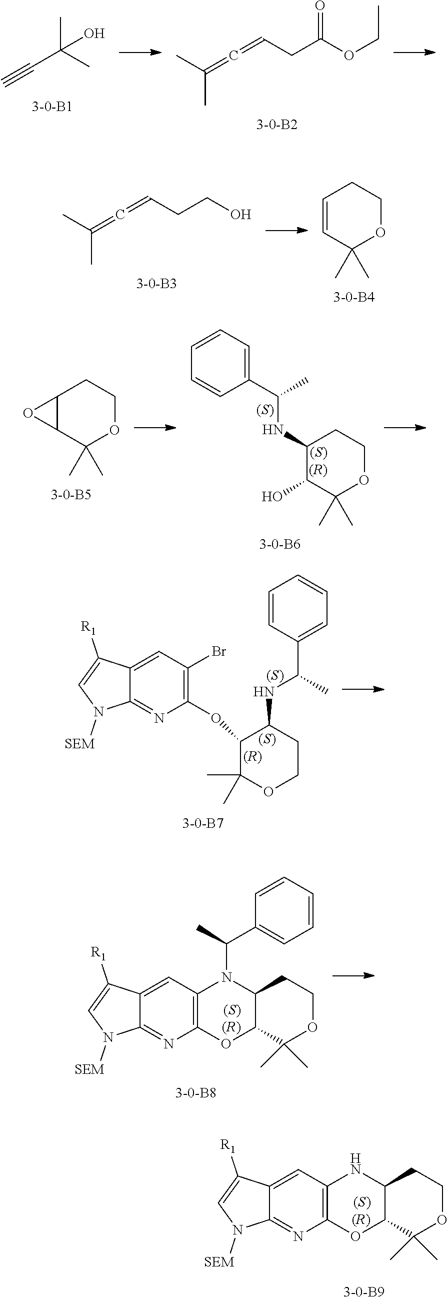

In Scheme 3-0-B, the starting material 3-0-B1 can be prepared by conventional procedures using appropriate compounds and reagents. The starting material 3-0-B1 can be converted to 3-0-B3 via a literature known reactions, which further undergoes a ring closing reaction to give 3-0-B4. After that, 3-0-B4 can be oxidized to 3-0-B5 readily, which is further converted to the intermediate 3-0-B6. Next, the intermediate 3-0-B6 undergoes a SNAr reaction to yield 3-0-B7, which can further go through an intramolecular coupling reaction to give 3-0-B8. Finally, the intermediate 3-0-B8 can be deprotected to afford the target compounds 3-0-B9.

Also, the target compounds can be synthesized by alternative methods but not limited to the above procedures.

A typical approach to synthesize the intermediate

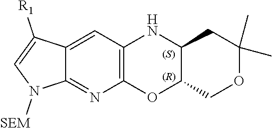

is described in Scheme 3-0—C below.

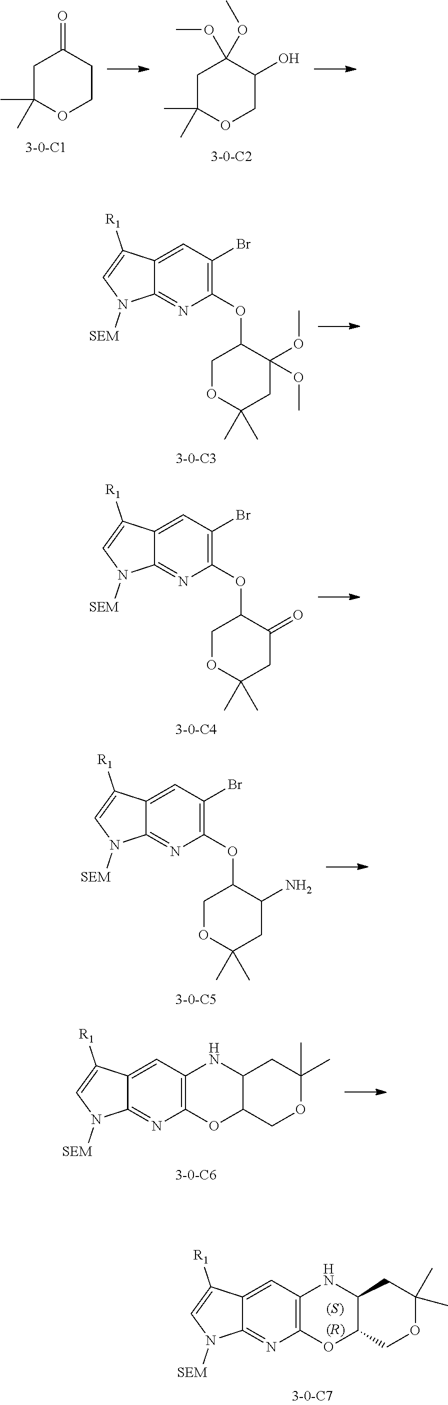

In Scheme 3-0-C, the starting material 3-0-C1 can be prepared by conventional procedures using appropriate compounds and reagents. The starting material 3-0-C1 can be converted to 3-0-C2 via a literature known reactions, which undergoes a SNAr reaction to yield 3-0-C3. After that, the intermediate 3-0-C3 is deprotected to give 3-0-C4, which can be converted to the intermediate 3-0-C5 via a reductive amination reaction. Finally, the intermediate 3-0-C5 goes through an intramolecular coupling reaction to afford the trans-racemate compounds 3-0-C6, which further go through a chiral separation to yield the target compounds 3-0-C7.

Also, the target compounds can be synthesized by alternative methods but not limited to the above procedures.

A typical approach to synthesize the intermediate

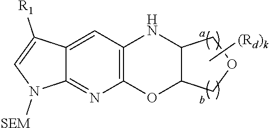

is described in Scheme 3-1 below.

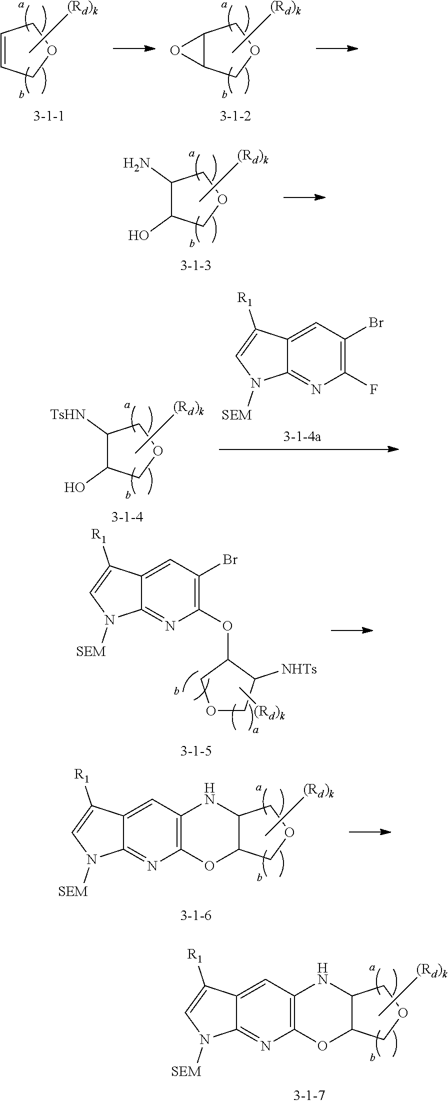

In Scheme 3-1, the starting material 3-1-1 can be prepared by conventional procedures using appropriate compounds and reagents. The starting material 3-1-1 can be converted to 3-1-2 readily, which further undergoes a ring opening reaction to give 3-1-3. After that, 3-1-3 can be converted to 3-1-4 via a general condition, and the reaction of 3-1-3 with 3-1-4a can generate intermediate 3-1-5. The intermediate 3-1-5 undergoes an intramolecular coupling reaction to give 3-1-6, which is further deprotected to afford 3-1-7.

Also, the target compounds can be synthesized by alternative methods but not limited to the above procedures.

A typical approach to synthesize the intermediate

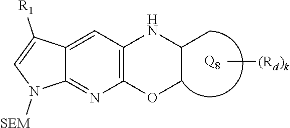

is described in Scheme 3-2 below.

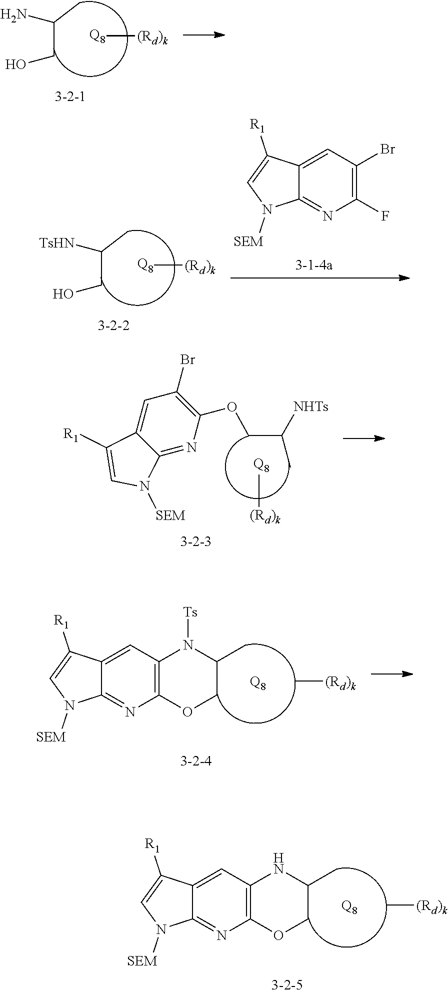

In Scheme 3-2, the starting material 3-2-1 can be prepared by conventional procedures using appropriate compounds and reagents. The starting material 3-2-1 can be converted to 3-2-2 readily, which further undergoes a SNAr reaction with 3-1-4a to give 3-2-3. After that, 3-2-3 undergoes an intramolecular coupling reaction to generate 3-2-4, which can be deprotected to afford 3-2-5.

Also, the target compounds can be synthesized by alternative methods but not limited to the above procedures.

A typical approach to synthesize the intermediate

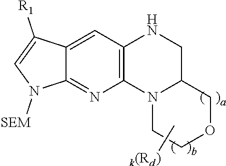

is described in Scheme 3-3 below.

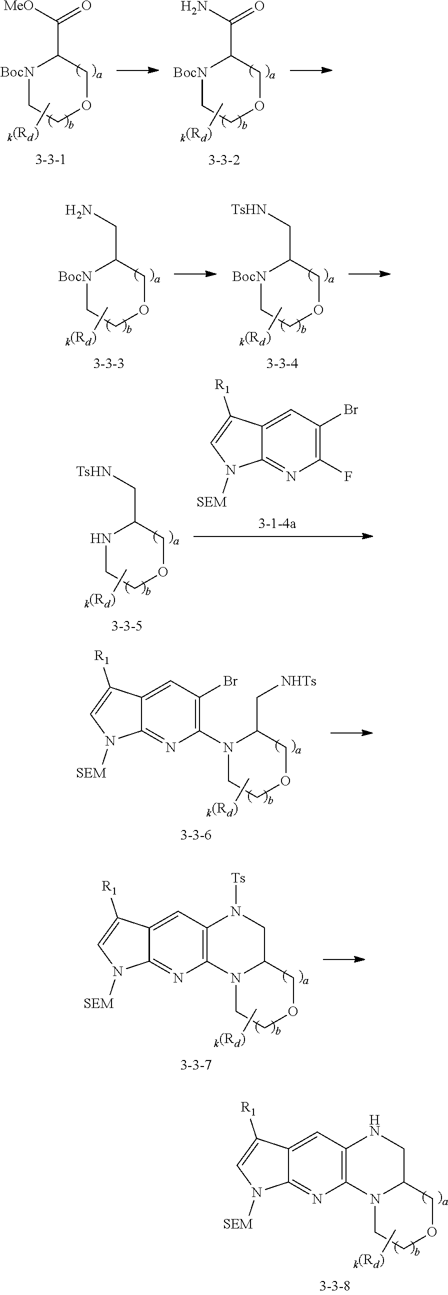

In Scheme 3-3, the starting material 3-3-1 can be converted to 3-3-3 through a two-step sequence of conventional reactions. After that, 3-3-3 can be converted to 3-3-4 readily, which can be deprotected to give 3-3-5. The reaction of 3-3-5 with 3-1-4a can generate 3-3-6, which can be converted to 3-3-7 via an intramolecular cyclization reaction. Finally, the deprotection of 3-3-7 can generate the target compounds 3-3-8.

Also, the target compounds can be synthesized by alternative methods but not limited to the above procedures.

A typical approach to synthesize the intermediate

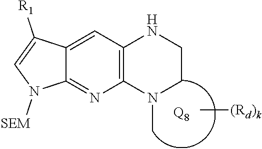

is described in Scheme 3-4 below.

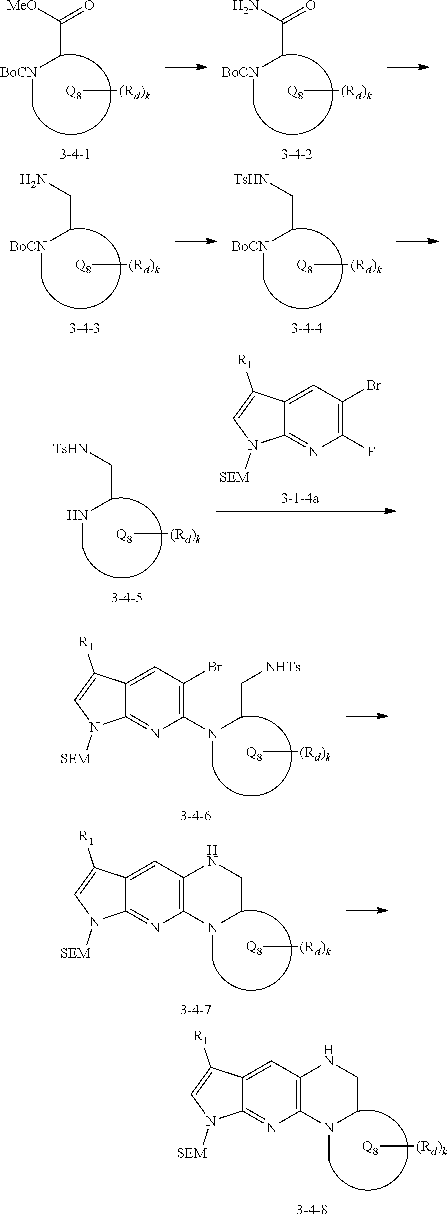

In Scheme 3-4, the starting material 3-4-1 can be converted to 3-4-3 through a two-step sequence of conventional reactions. After that, 3-4-3 can be converted to 3-4-4 readily, which can be deprotected to give 3-4-5. The reaction of 3-4-5 with 3-1-4a can generate 3-4-6, which can be converted to 3-4-7 via an intramolecular cyclization reaction. Finally, the deprotection of 3-4-7 can generated the target compounds 3-4-8.

Also, the target compounds can be synthesized by alternative methods but not limited to the above procedures.

A typical approach to synthesize the intermediate

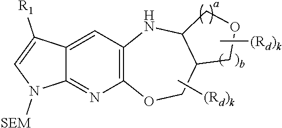

is described in Scheme 3-5 below.

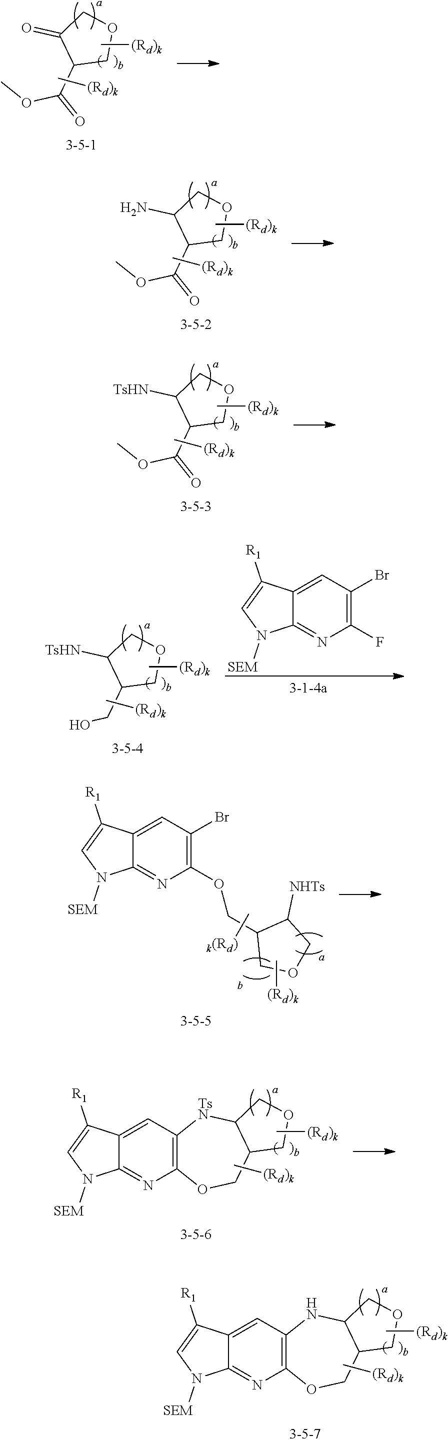

In Scheme 3-5, the starting material 3-5-1 can be converted to 3-5-2 via reductive amination reaction, and then 3-5-2 can be converted to 3-5-3 readily. After that, 3-5-3 can be reduced to 3-5-4. The reaction of 3-5-4 with 3-1-4a can generate 3-5-5, which can be converted to 3-5-6 via an intramolecular cyclization reaction. Finally, the deprotection of 3-5-6 can generate the target compounds 3-5-7.

Also, the target compounds can be synthesized by alternative methods but not limited to the above procedures.

A typical approach to synthesize the intermediate

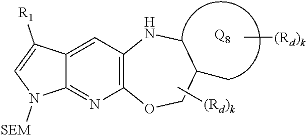

is described in Scheme 3-6 below.

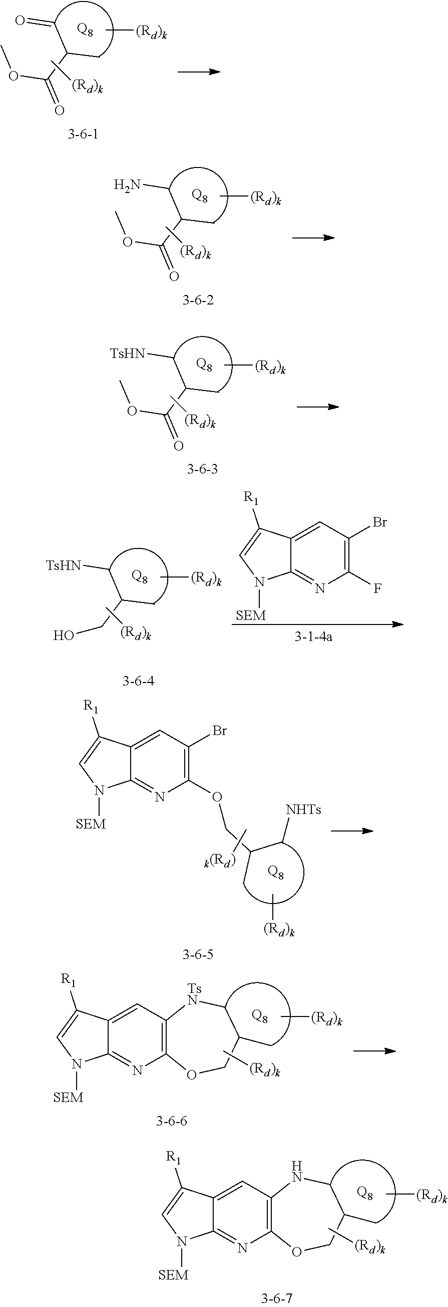

In Scheme 3-6, the starting material 3-6-1 can be converted to 3-6-2 via reductive amination reaction, and then 3-6-2 can be converted to 3-6-3 readily. After that, 3-6-3 can be reduced to 3-6-4. The reaction of 3-6-4 with 3-1-4a can generate 3-6-5, which can be converted to 3-6-6 via an intramolecular cyclization reaction. Finally, the deprotection of 3-6-6 can generate the target compounds 3-6-7.

Also, the target compounds can be synthesized by alternative methods but not limited to the above procedures.

A typical approach to synthesize the intermediate

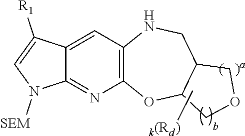

is described in Scheme 3-7 below.

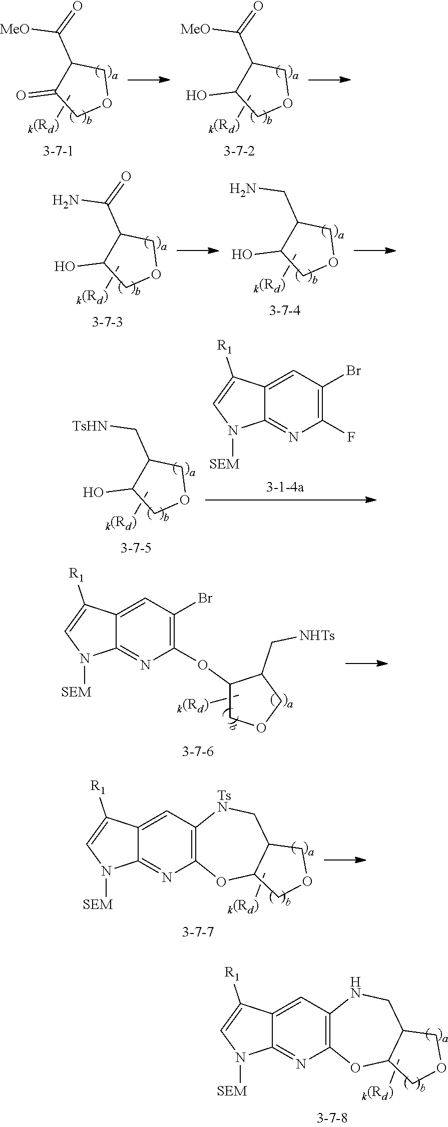

In Scheme 3-7, the starting material 3-7-1 can be reduced to give 3-7-2, and then 3-7-2 can be converted to 3-7-3 readily. After that, 3-7-3 can be converted to 3-7-5 through a two-step sequence of conventional reactions. The reaction of 3-7-5 with 3-1-4a can generate 3-7-6, which can be converted to 3-7-7 via an intramolecular cyclization reaction. Finally, the deprotection of 3-7-7 can generate the target compounds 3-7-8.

Also, the target compounds can be synthesized by alternative methods but not limited to the above procedures.

A typical approach to synthesize the intermediate

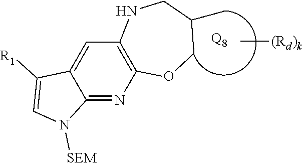

is described in Scheme 3-8 below.

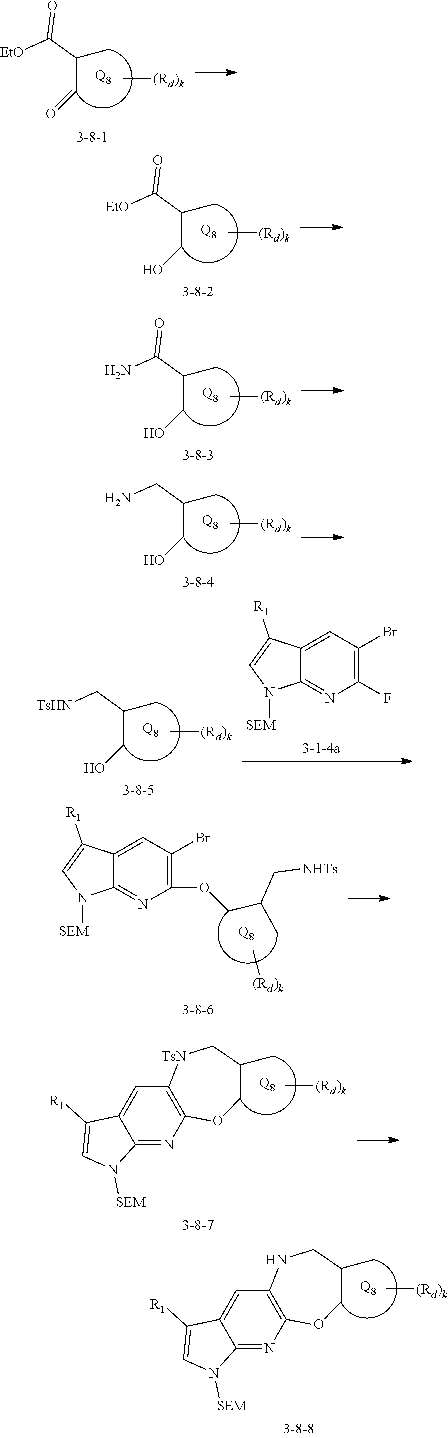

In Scheme 3-8, the starting material 3-8-1 can be reduced to give 3-7-2, and then 3-8-2 can be converted to 3-8-3 readily. After that, 3-8-3 is further converted to 3-8-5 through a two-step sequence of conventional reactions. The reaction of 3-8-5 with 3-1-4a can generate 3-8-6, which can be converted to 3-8-7 via an intramolecular cyclization reaction. Finally, the deprotection of 3-7-7 can generate the target compounds 3-8-8.

Also, the target compounds can be synthesized by alternative methods but not limited to the above procedures.

A typical approach to synthesize the intermediate

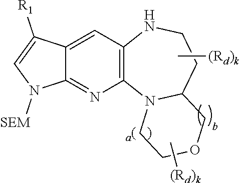

is described in Scheme 3-9 below.

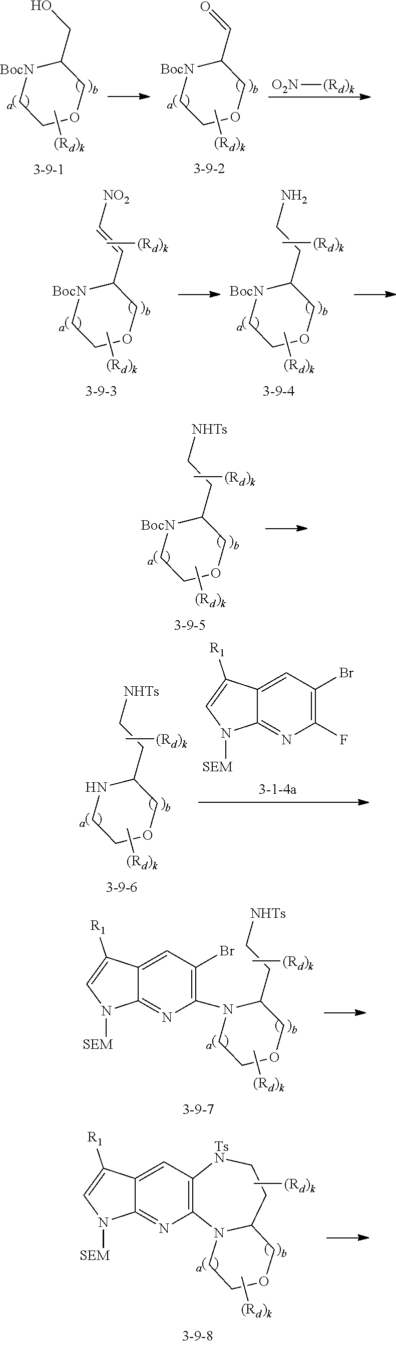

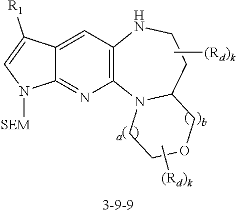

In Scheme 3-9, the starting material 3-9-1 can be oxidized to give 3-9-2, and then 3-9-2 can be converted to 3-9-3 readily. After that, the hydrogenation of 3-9-3 can give 3-9-4, which is further converted to 3-9-6 through a two-step sequence of conventional reactions. The reaction of 3-9-6 with 3-1-4a can generate 3-9-7, which can be converted to 3-9-8 via an intramolecular cyclization reaction. Finally, the deprotection of 3-9-8 can generate the target compounds 3-9-9.

Also, the target compounds can be synthesized by alternative methods but not limited to the above procedures.

A typical approach to synthesize the intermediate

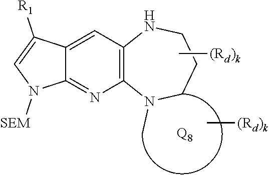

is described in Scheme 3-10 below.

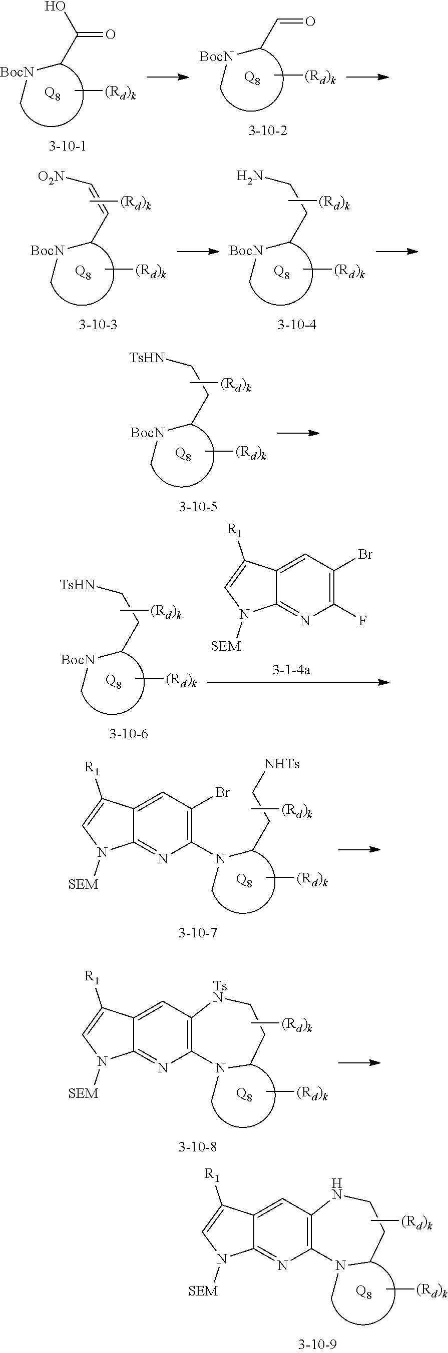

In Scheme 3-10, the starting material 3-10-1 can be reduced to give 3-10-2, and then 3-10-2 can be converted to 3-10-3 readily. After that, the hydrogenation of 3-10-3 can give 3-10-4, which can be converted to 3-10-6 through a two-step sequence of conventional reactions. The reaction of 3-10-6 with 3-1-4a can generate 3-10-7, which can be converted to 3-10-8 via an intramolecular cyclization reaction. Finally, the deprotection of 3-10-8 can generate the target compounds 3-10-9.

Also, the target compounds can be synthesized by alternative methods but not limited to the above procedures.

Similarly, the intermediate of

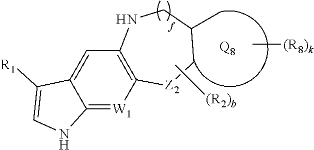

with different Z₂, f, k, R_{d} and Q₈ can be prepared by the method similar to the Scheme 3-1 to 3-10 by using appropriate staring materials, and intermediates.

Similarly, the compounds of

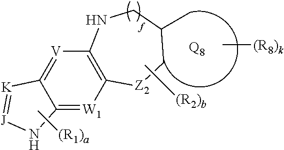

can be prepared by schemes similar to the Scheme 3-1 to 3-10 by using appropriate staring materials and intermediates.

Similarly, the compounds of

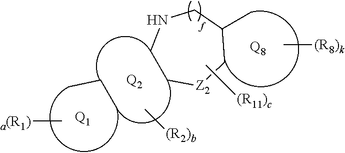

can be prepared by schemes similar to the Scheme 3-1 to 3-10 by using appropriate staring materials and intermediates.

A typical approach to synthesize the compounds of

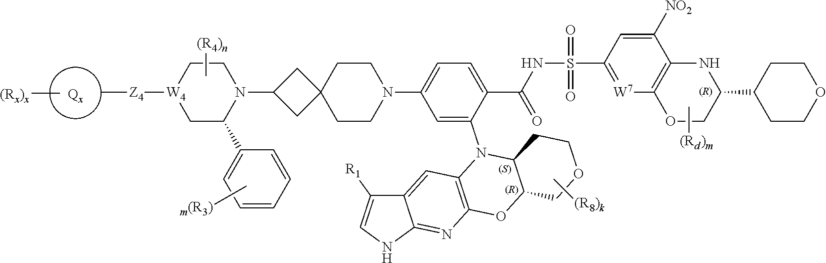

is described in Scheme A below.

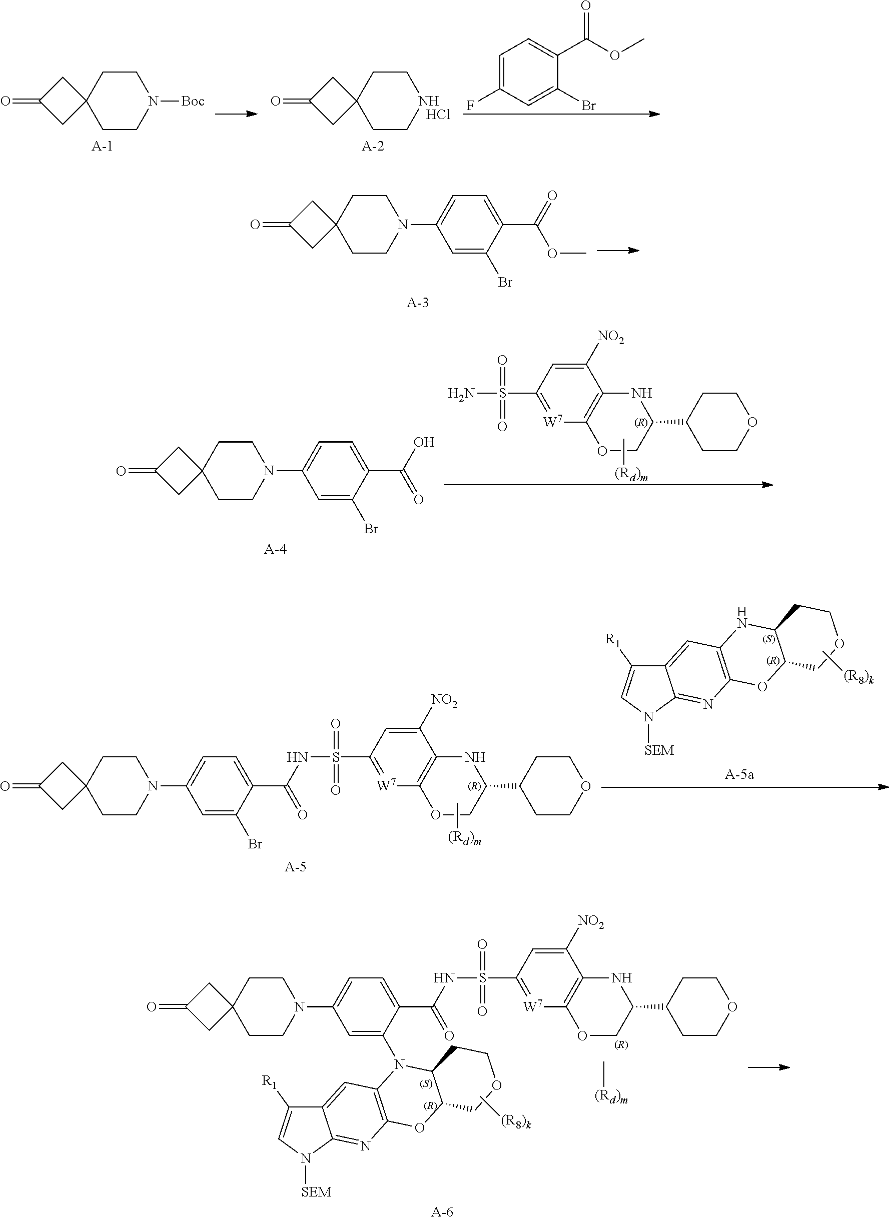

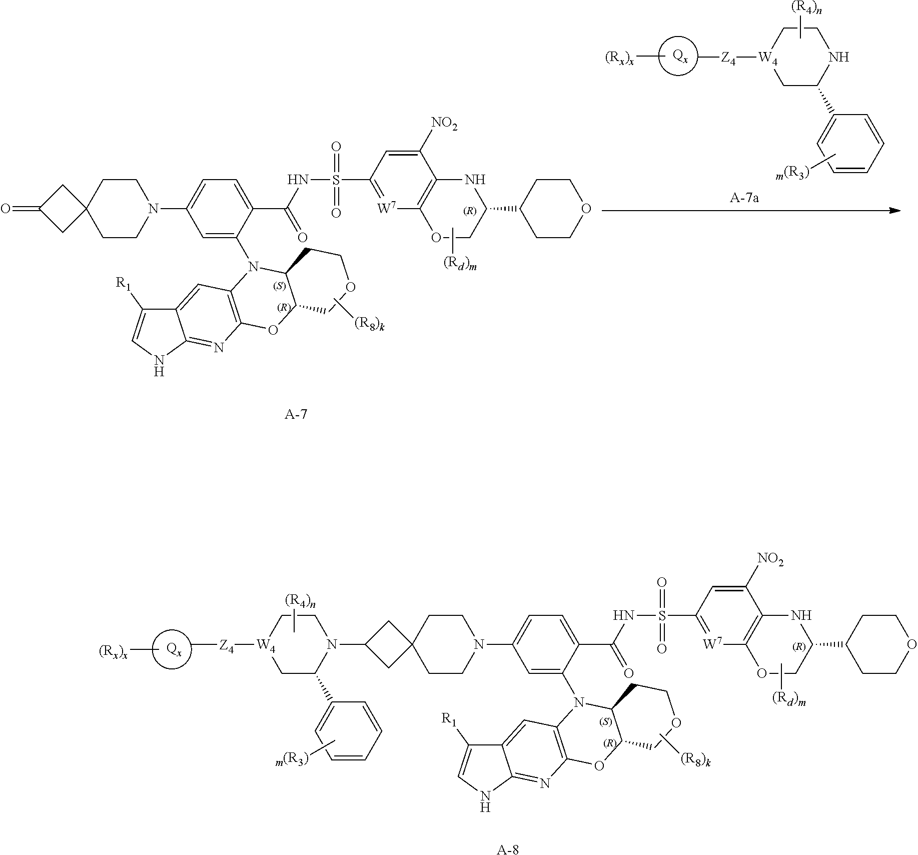

In Scheme A, the starting material A-1 can be deprotected to give A-2, and then A-2 can react with A-2a to afford A-3 readily. After that, the hydrolysis of A-3 can yield A-4, which is further converted to A-5. The coupling reaction of A-5 with A-5a can generate A-6, which can be converted to A-7. Finally, A-7 can react with A-7a to provide the target compounds A-8.

Also, the target compounds can be synthesized by alternative methods but not limited to the above procedures.

A typical approach to synthesize the compounds of

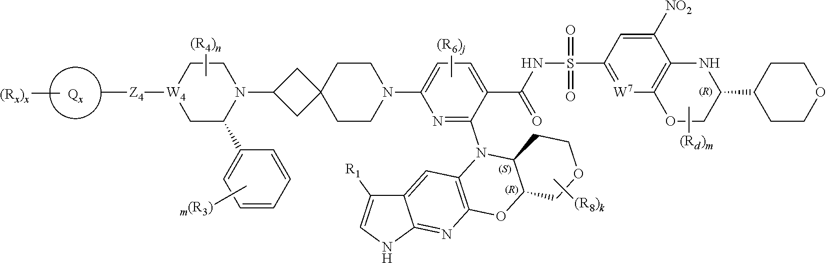

is described in Scheme B below.

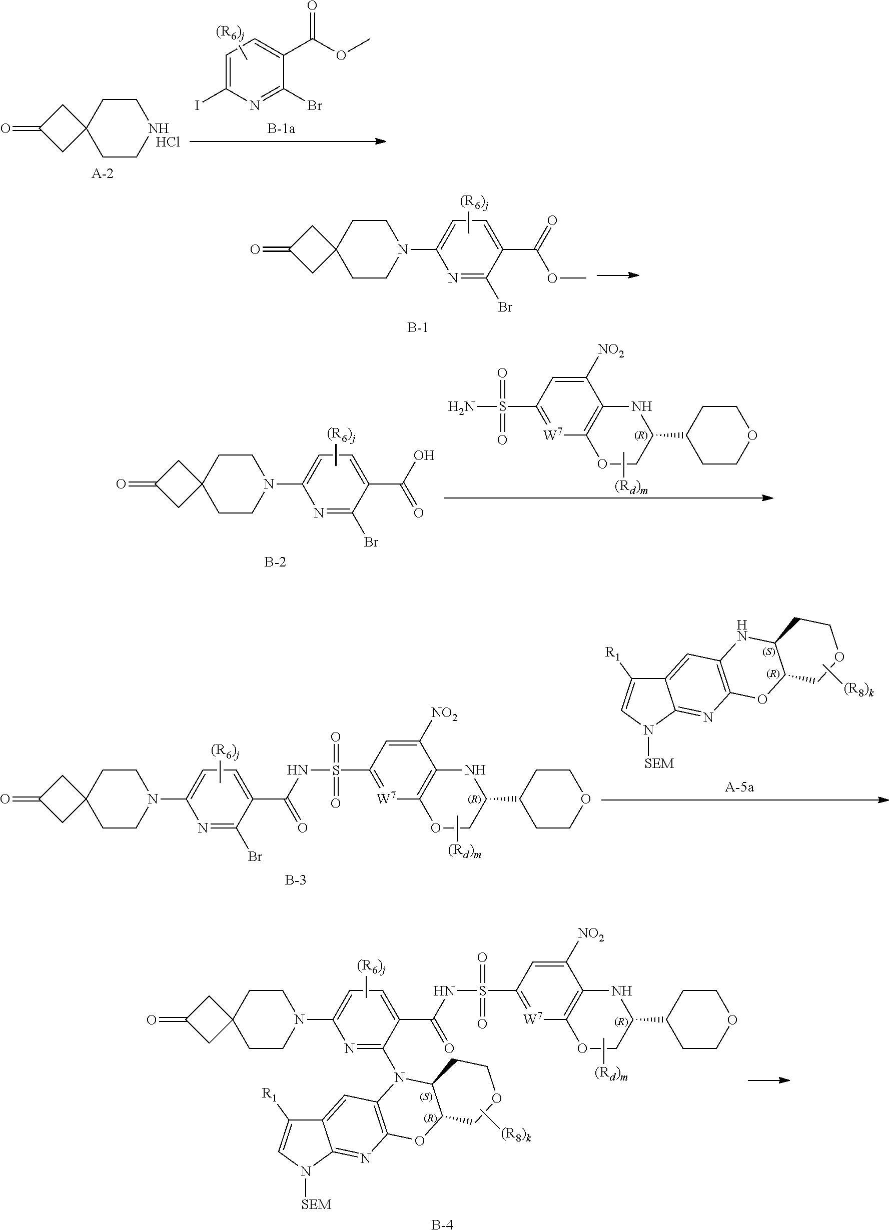

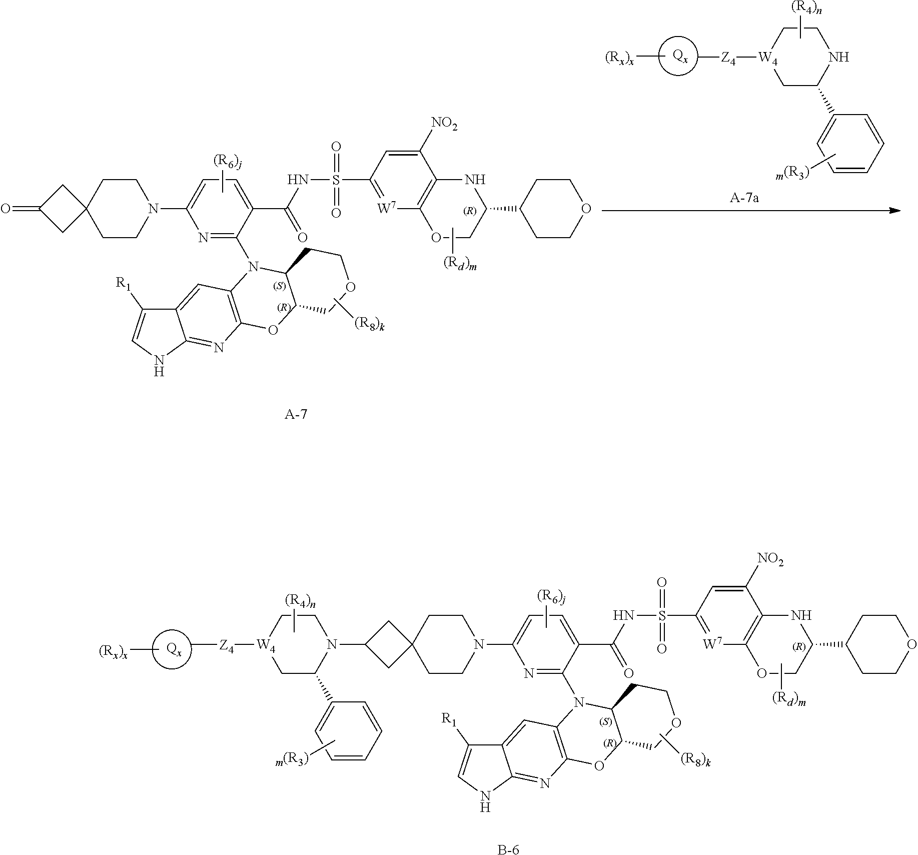

In Scheme B, the starting material A-2 can react with B-1a to afford B-1 readily. After that, the hydrolysis of B-1 to afford B-2, which can be further converted to B-3. The coupling reaction of B-3 with A-5a can generate B-4, which is deprotected to give B-5. Finally, the intermediate B-5 can go through a reductive amination reaction with A-7a to provide the target compounds B-6.

Also, the target compounds can be synthesized by alternative methods but not limited to the above procedures.

A typical approach to synthesize the compounds of

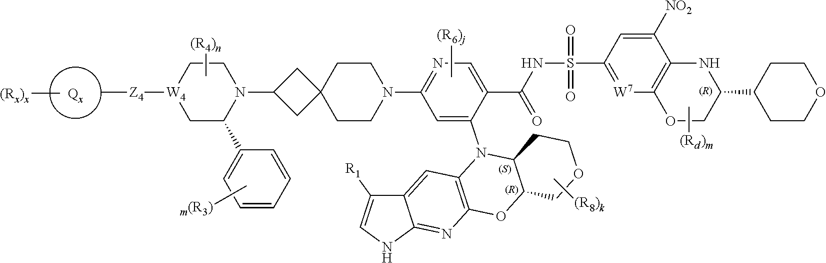

is described in Scheme C below.

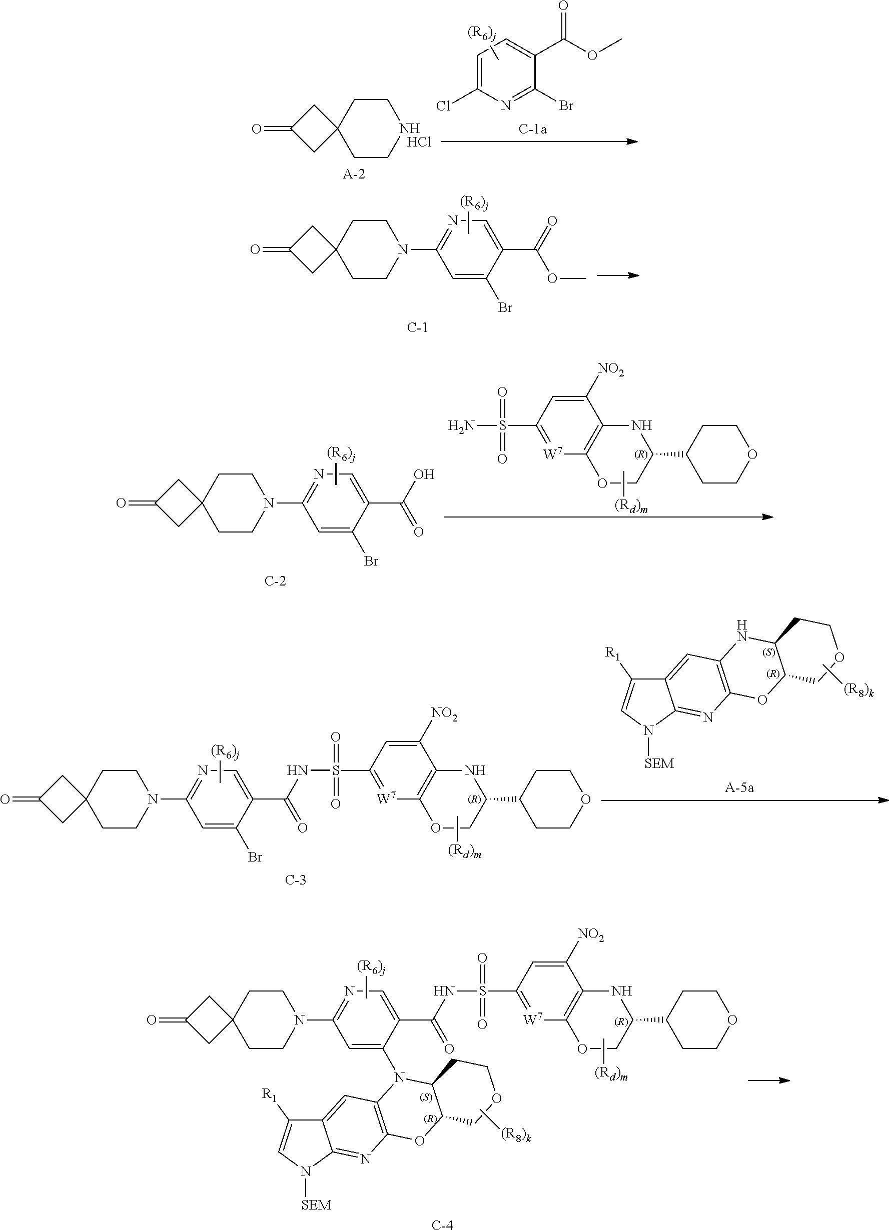

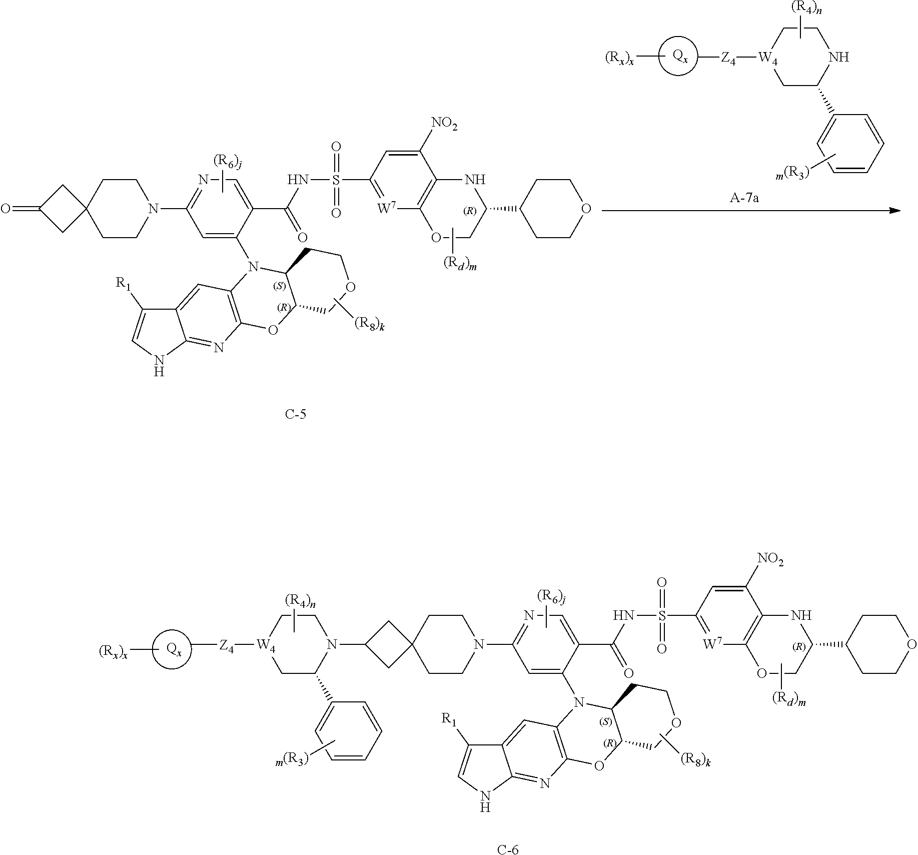

In Scheme C, the starting material A-2 can react with C-1a to afford C-1 readily. After that, the hydrolysis of C-1 to afford C-2, which can be further converted to C-3. The coupling reaction of C-3 with A-5a can generate C-4, which is deprotected to give C-5. Finally, the intermediate C-5 can go through a reductive amination reaction with A-7a to provide the target compounds C-6.

Also, the target compounds can be synthesized by alternative methods but not limited to the above procedures.

A typical approach to synthesize the compounds of

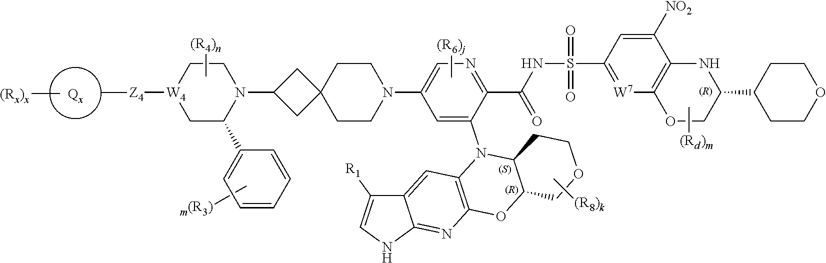

is described in Scheme D below.

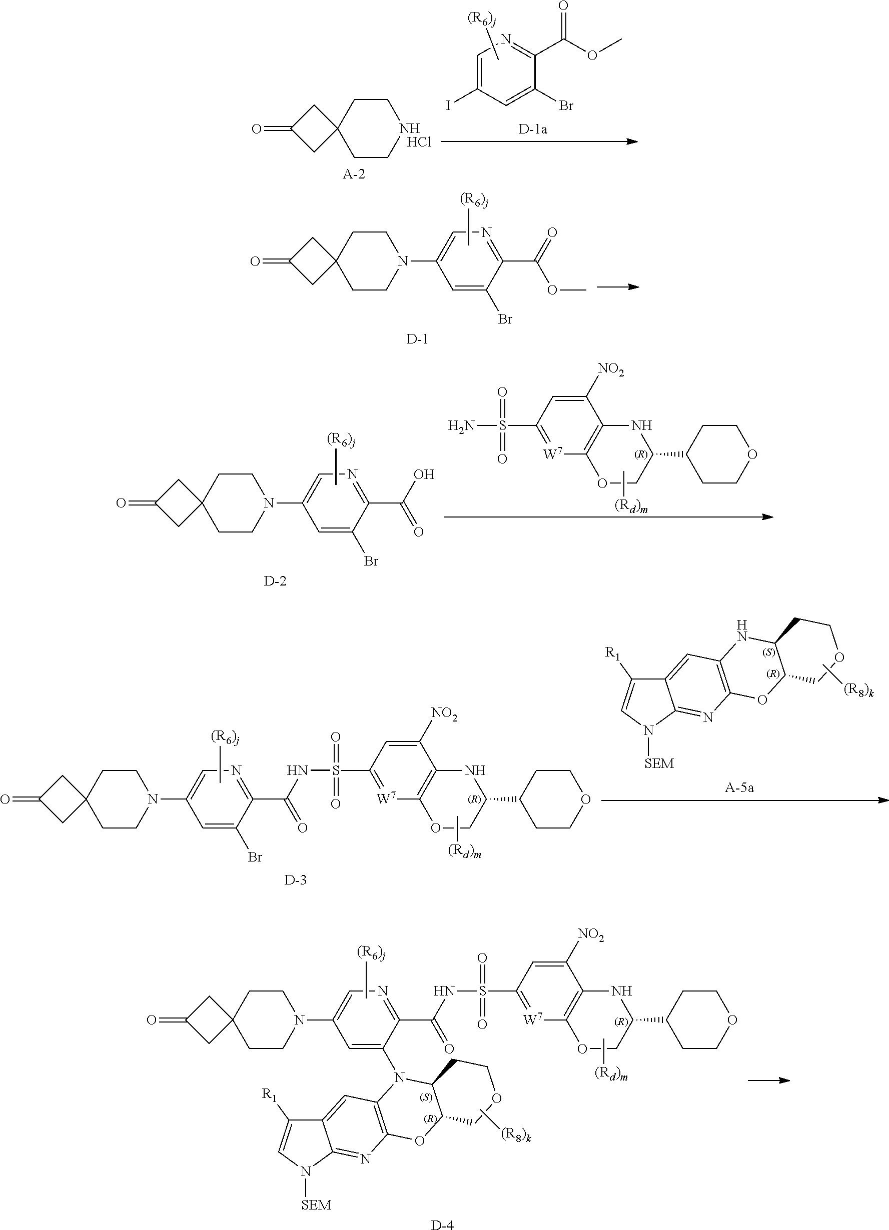

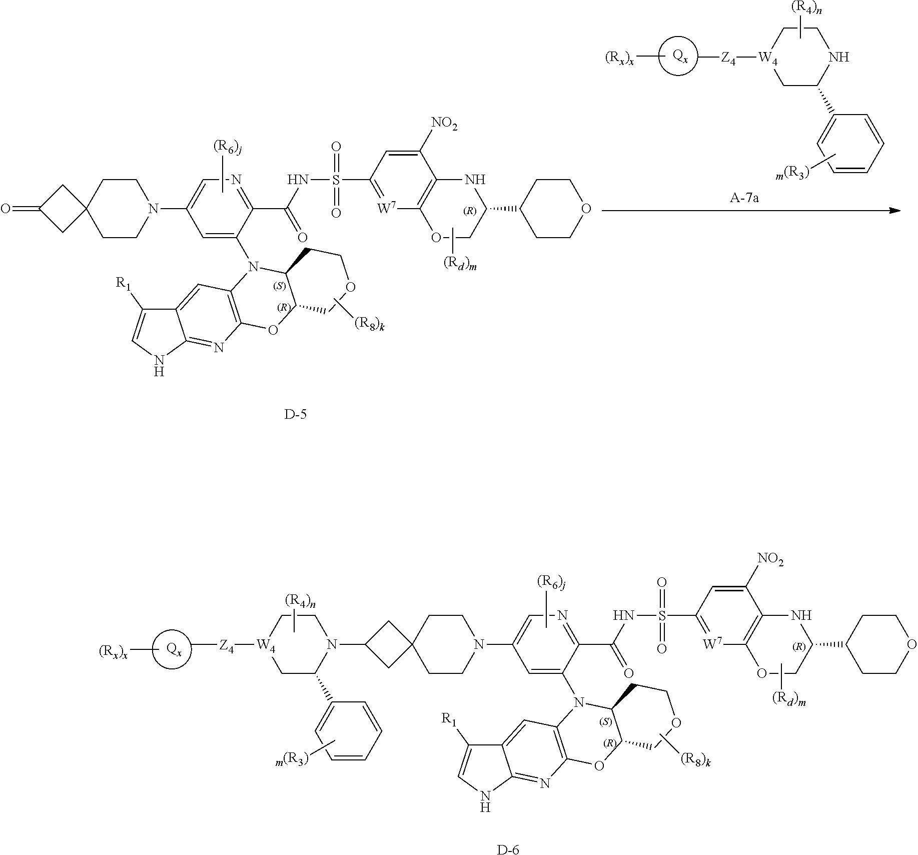

In Scheme D, the starting material A-2 can react with D-1a to afford D-1 readily. After that, the hydrolysis of D-1 to afford D-2, which can be further converted to D-3. The coupling reaction of D-3 with A-5a can generate D-4, which is deprotected to give D-5. Finally, the intermediate D-5 can go through a reductive amination reaction with A-7a to provide the target compounds D-6.

Also, the target compounds can be synthesized by alternative methods but not limited to the above procedures.

A typical approach to synthesize the compounds of

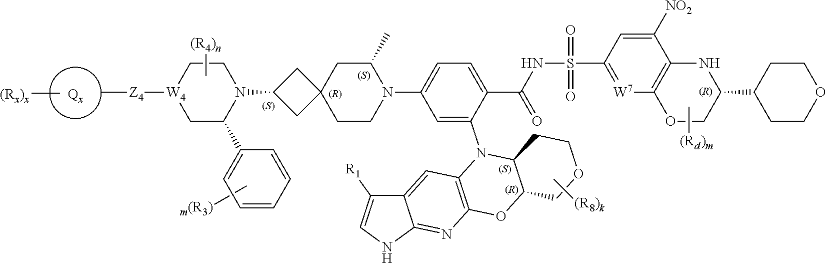

is described in Scheme E below.

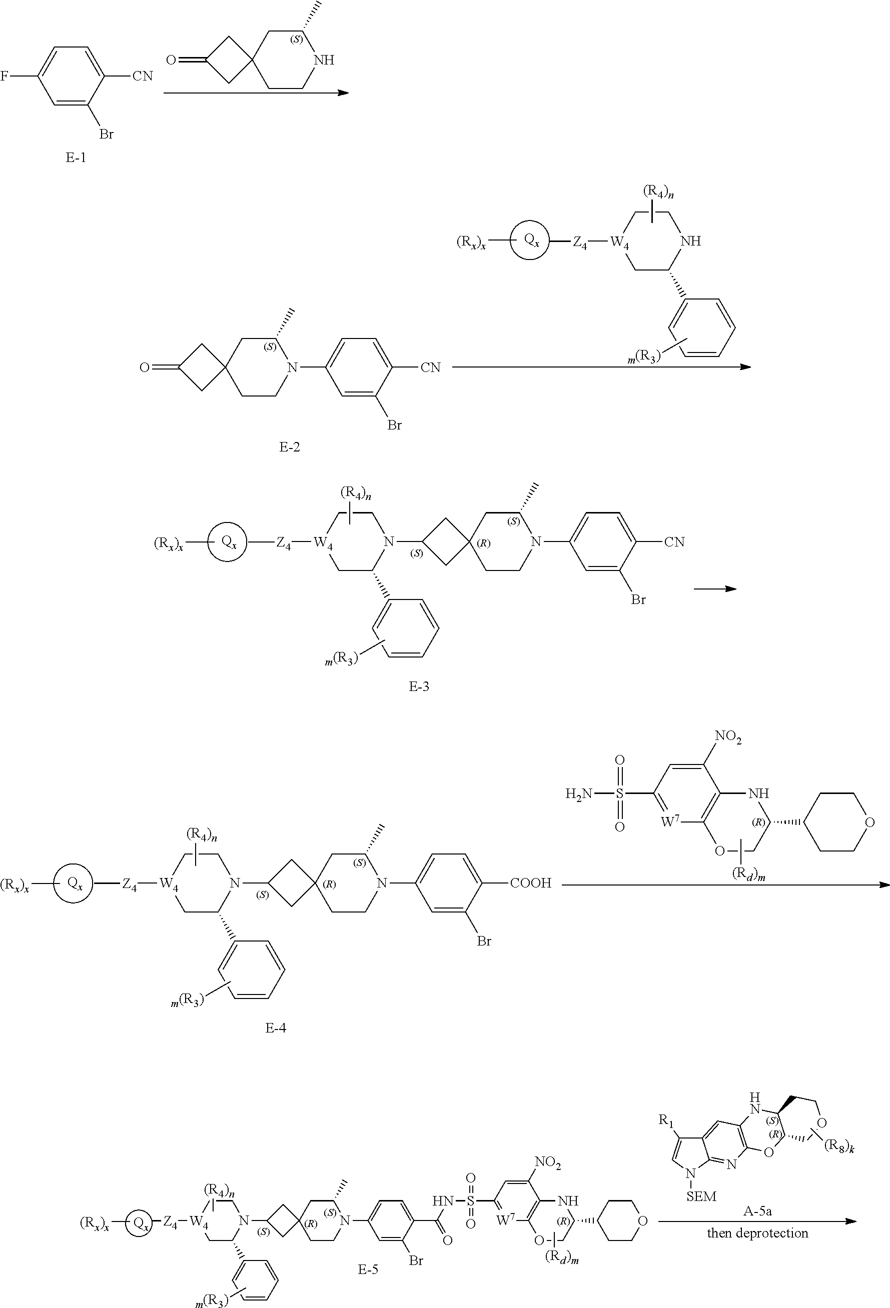

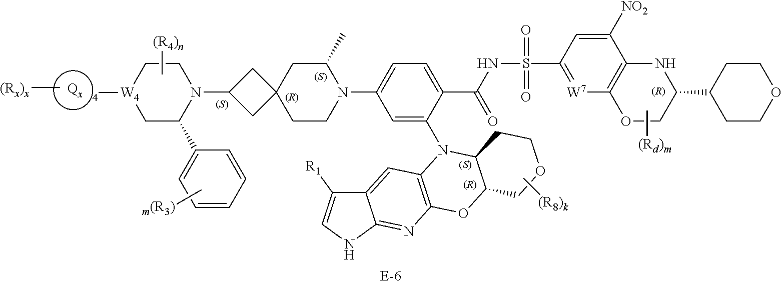

In Scheme E, the starting material E-1 can be prepared by conventional procedures using appropriate compounds and reagent. The starting material E-1 can be converted to intermediate E-2 through SNAr substitution reaction. After that, the intermediate E-2 can go through a reductive amination reaction with A-7a to provide the intermediate E-3, which can be hydrolyzed to E-4 readily. Finally, E-4 can be converted to E-5, which can couple with A-5a to yield the target compound E-6.

Also, the target compounds can be synthesized by alternative methods but not limited to the above procedures.

A typical approach to synthesize the compounds of

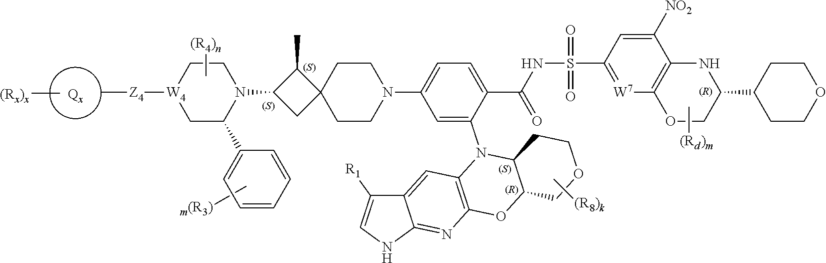

is described in Scheme F below.

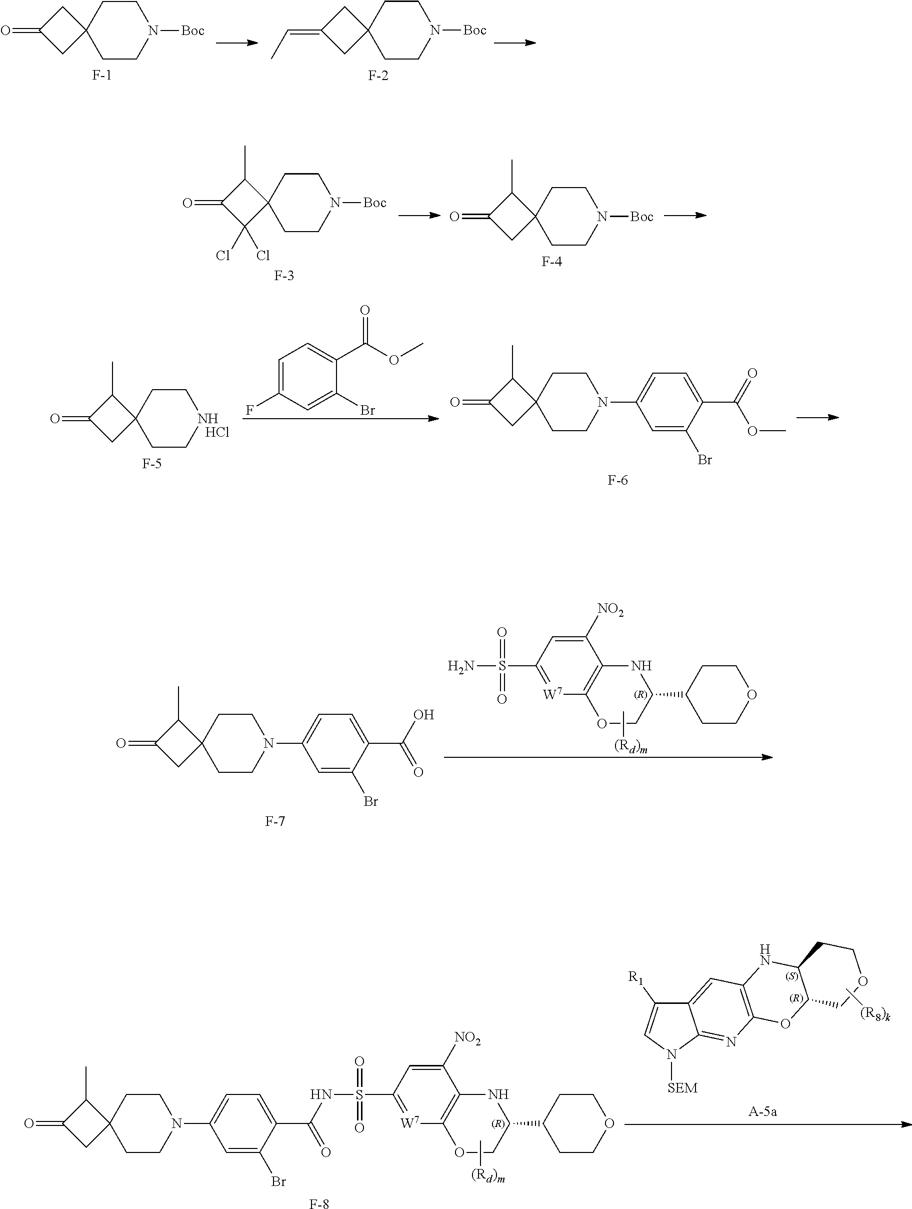

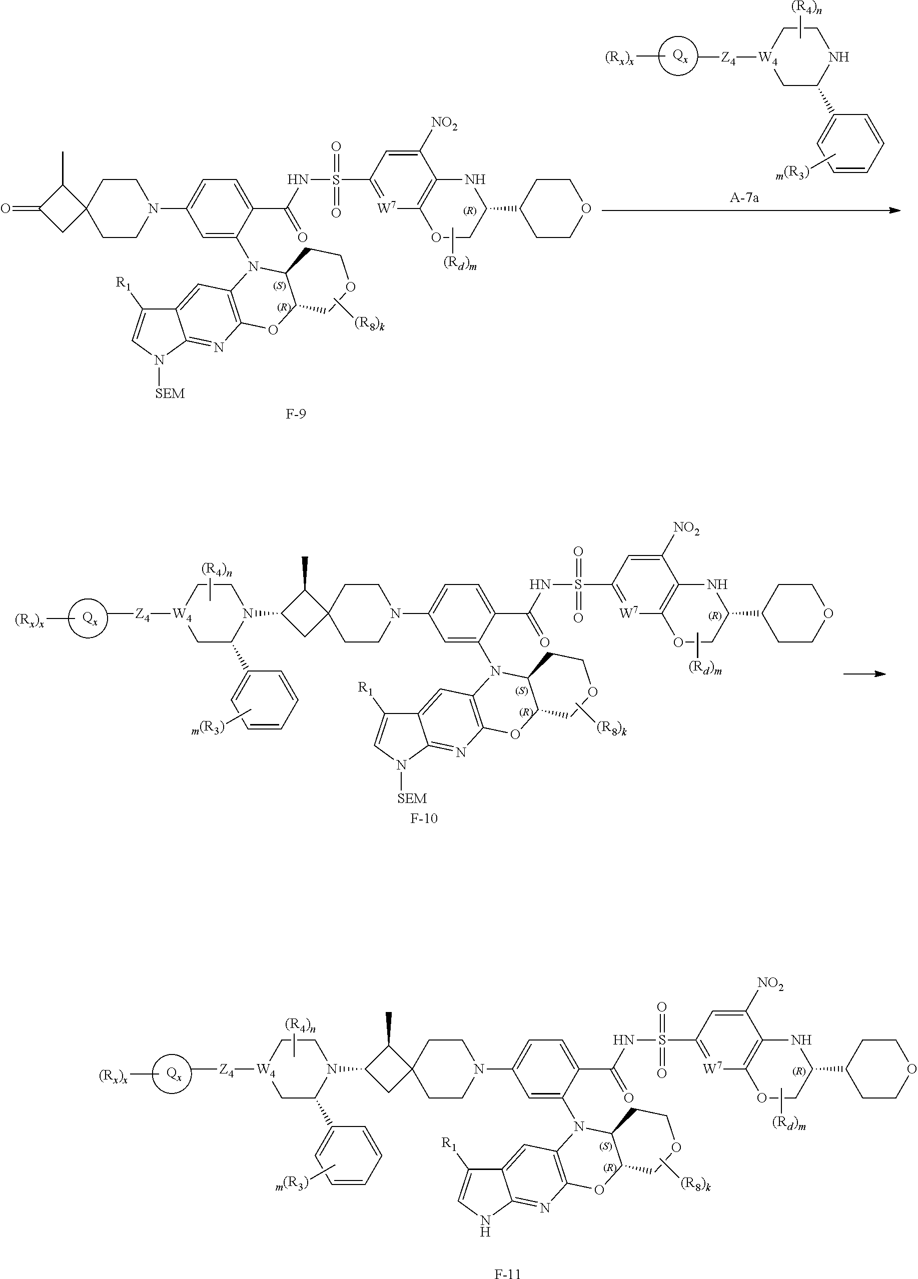

In Scheme F, the starting material F-1 can be prepared by conventional procedures using appropriate compounds and reagent. The starting material F-1 can be converted to intermediate F-2 through Wittig reaction.

After that, the intermediate E-2 can go through a [2+2]cyclization to afford F-3, which is de-chlorination to give F-4. The intermediate F-4 is deprotected to provide F-5, which can converted to F-6 readily. Next, the hydrolysis of F-6 can afford F-7, which can be further converted to F-8. The coupling reaction of F-8 with A-5a can generate F-9. Finally, the intermediate F-9 can go through a reductive amination reaction with A-7a to provide F-10, which is deprotected to yield the target compounds F-11.

Also, the target compounds can be synthesized by alternative methods but not limited to the above procedures.

A typical approach to synthesize the compounds of

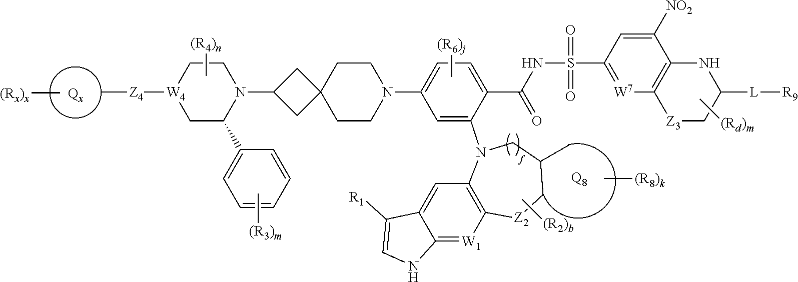

is described in Scheme G below.

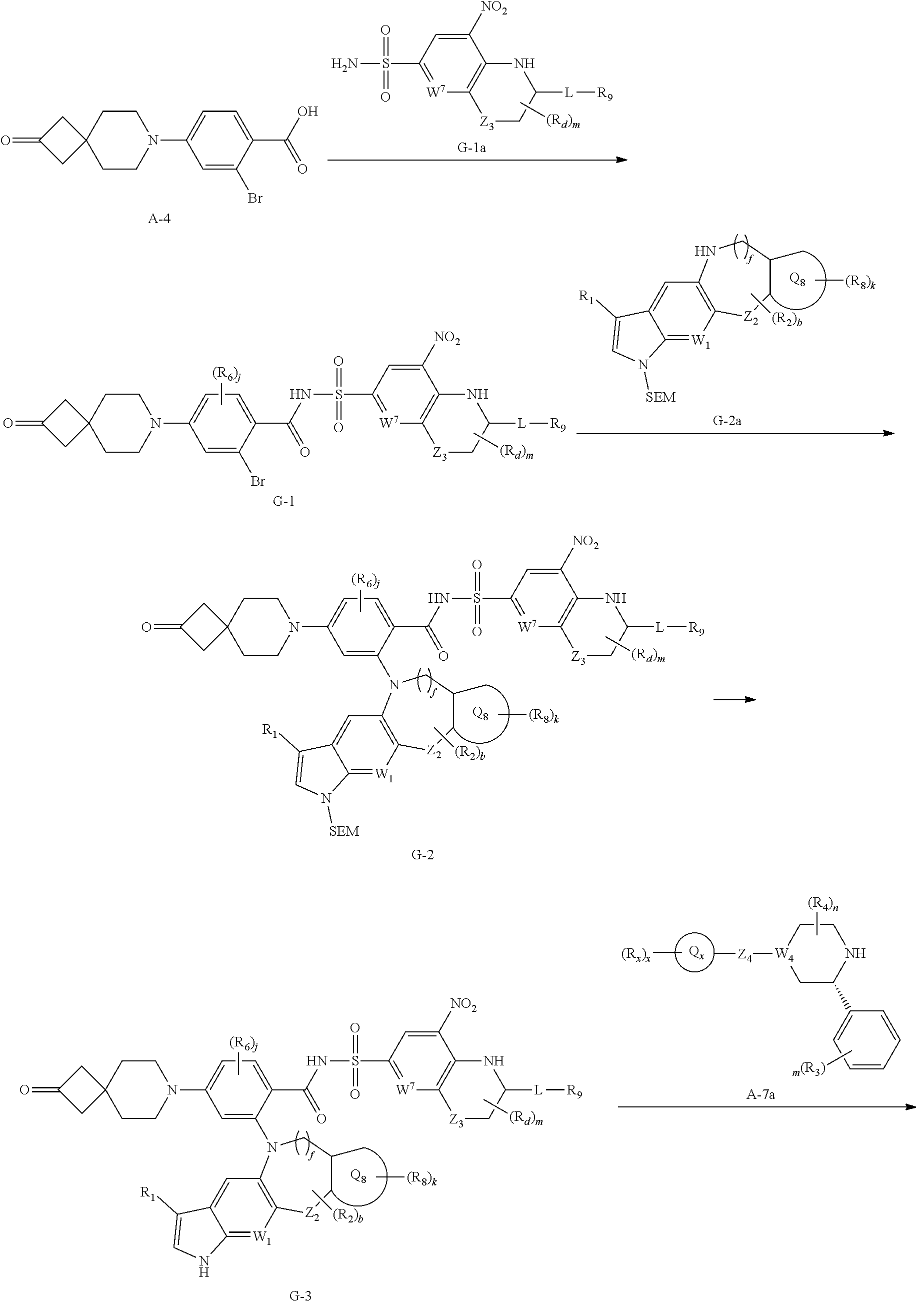

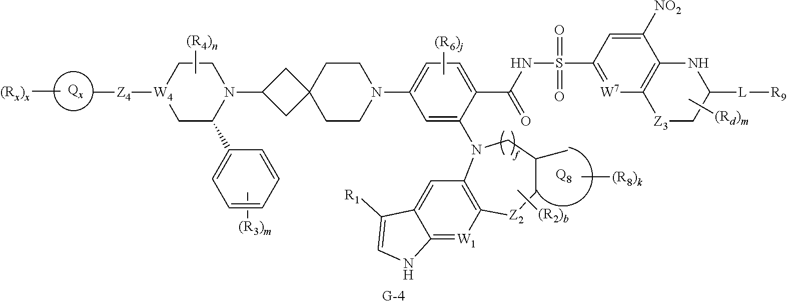

In Scheme G, the starting material A-4 can react with G-1a to afford G-1 readily. After that, the coupling reaction of G-1 with G-2a can generate G-2, which is deprotected to give G-3. Finally, the intermediate G-3 can go through a reductive amination reaction with A-7a to provide the target compounds G-4.

Also, the target compounds can be synthesized by alternative methods but not limited to the above procedures.

A typical approach to synthesize the compounds of

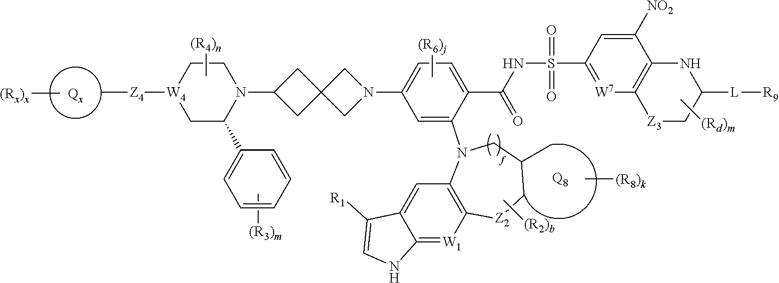

is described in Scheme H below.

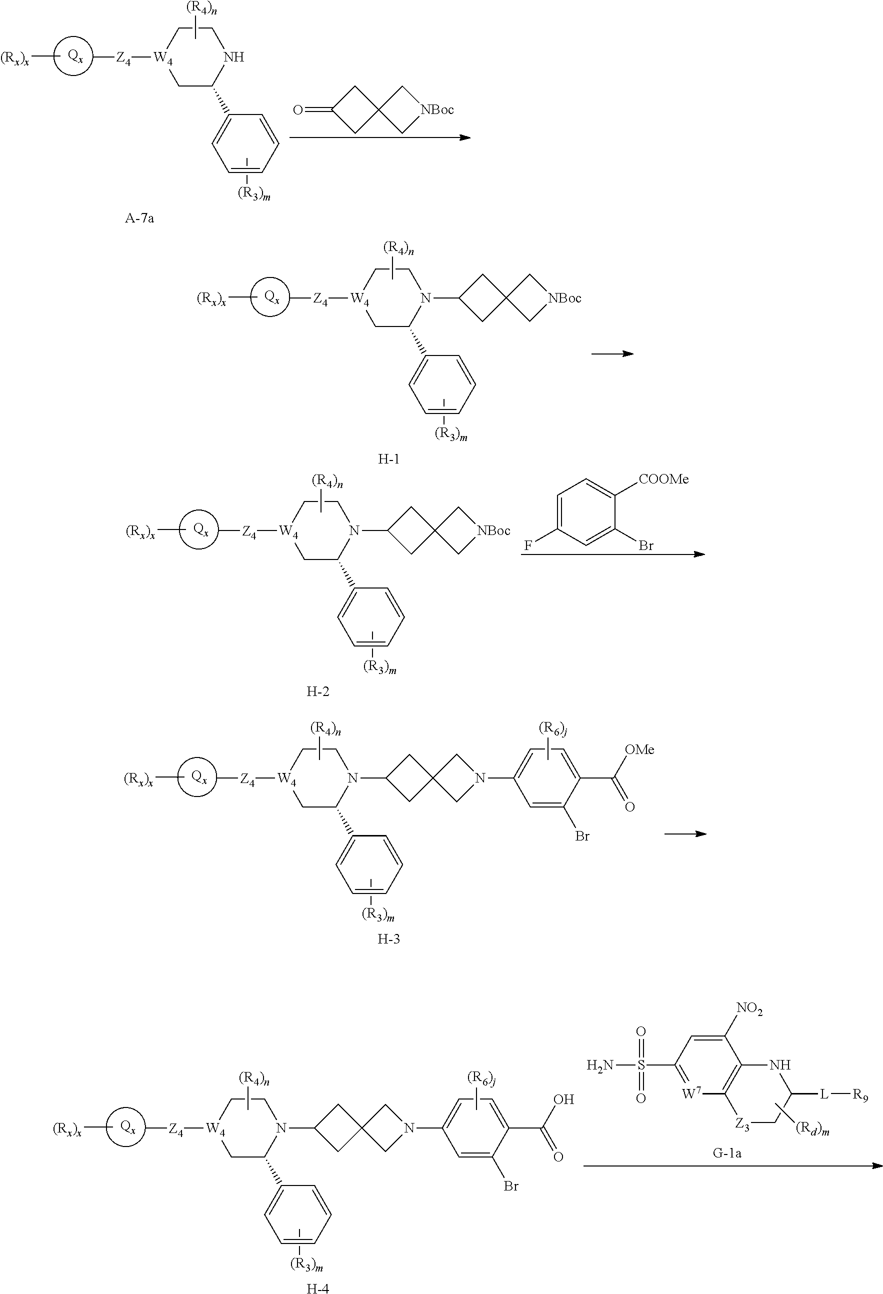

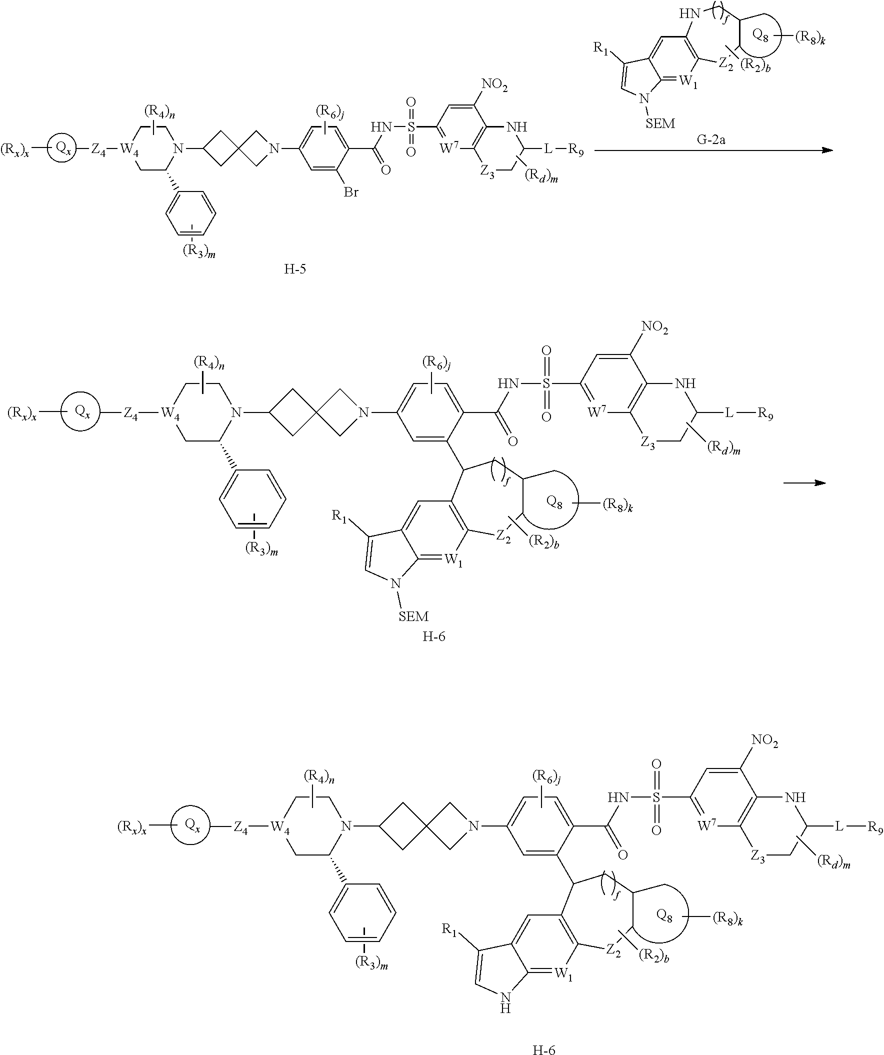

In Scheme H, the starting material A-1a can be converted to H-1 via reductive amination reaction, which is deprotected to give H-2. The intermediate H-2 can go through a SNAr substitution reaction to afford H-3. After that, the hydrolysis of H-3 can yield H-4, which is further converted to H-5. Finally, the coupling reaction of H-5 with G-2a can generate H-6, which is deprotected to provide the target compounds H-7.

Also, the target compounds can be synthesized by alternative methods but not limited to the above procedures.

A typical approach to synthesize the compounds of

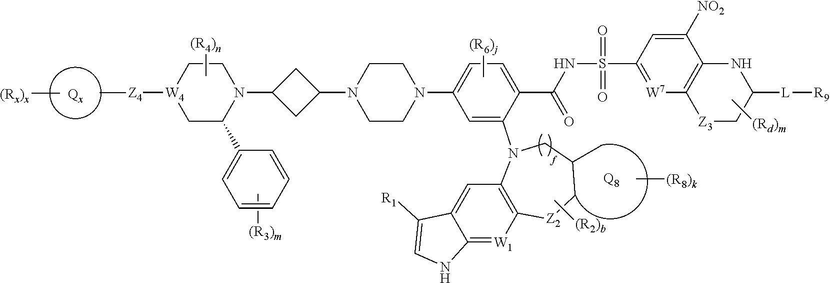

is described in Scheme I below.

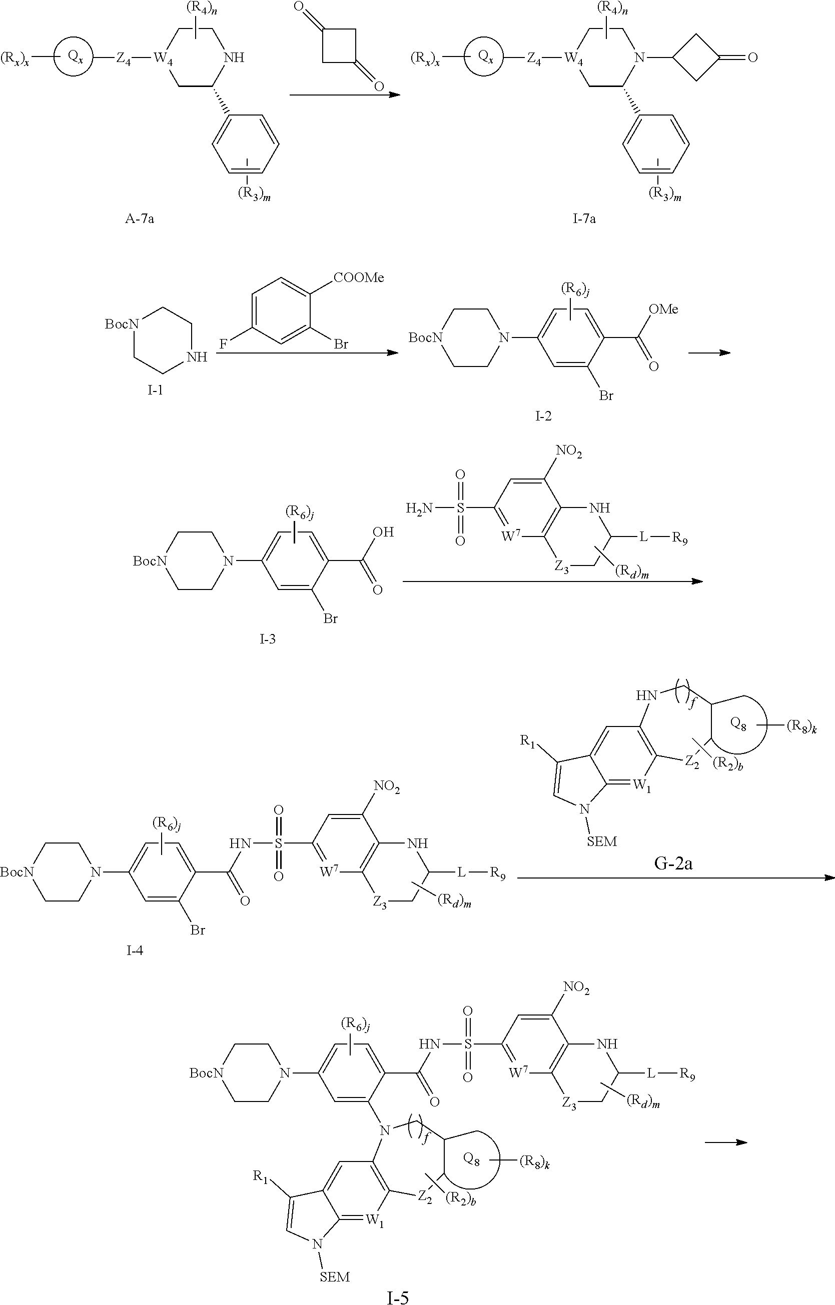

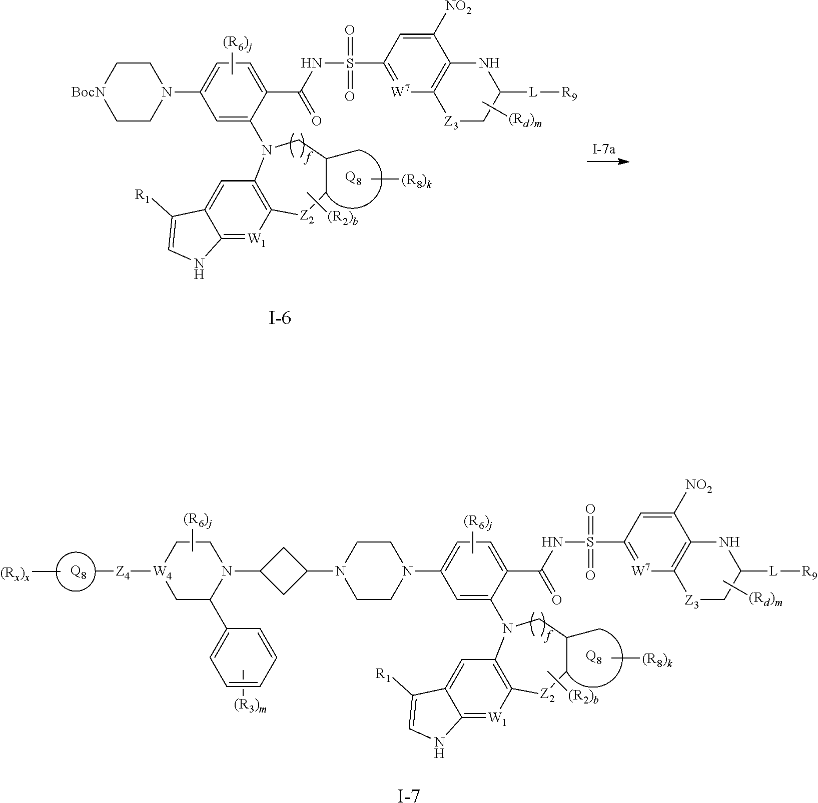

In Scheme I, the starting material A-7a can be converted to I-1a via reductive amination reaction. The intermediate I-1 can go through a SNAr substitution reaction to afford I-2. After that, the hydrolysis of I-2 can yield I-3, which is further converted to I-4. Next, the coupling reaction of I-4 with G-2a can generate I-5, which is deprotected to provide the target compounds I-6. Finally, the intermediate I-6 can go through a reductive amination reaction with I-7a to provide the target compounds I-7.

Also, the target compounds can be synthesized by alternative methods but not limited to the above procedures.

A typical approach to synthesize the compounds of

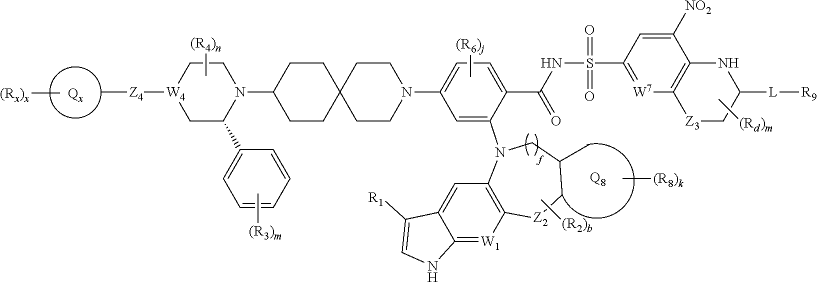

is described in Scheme J below.

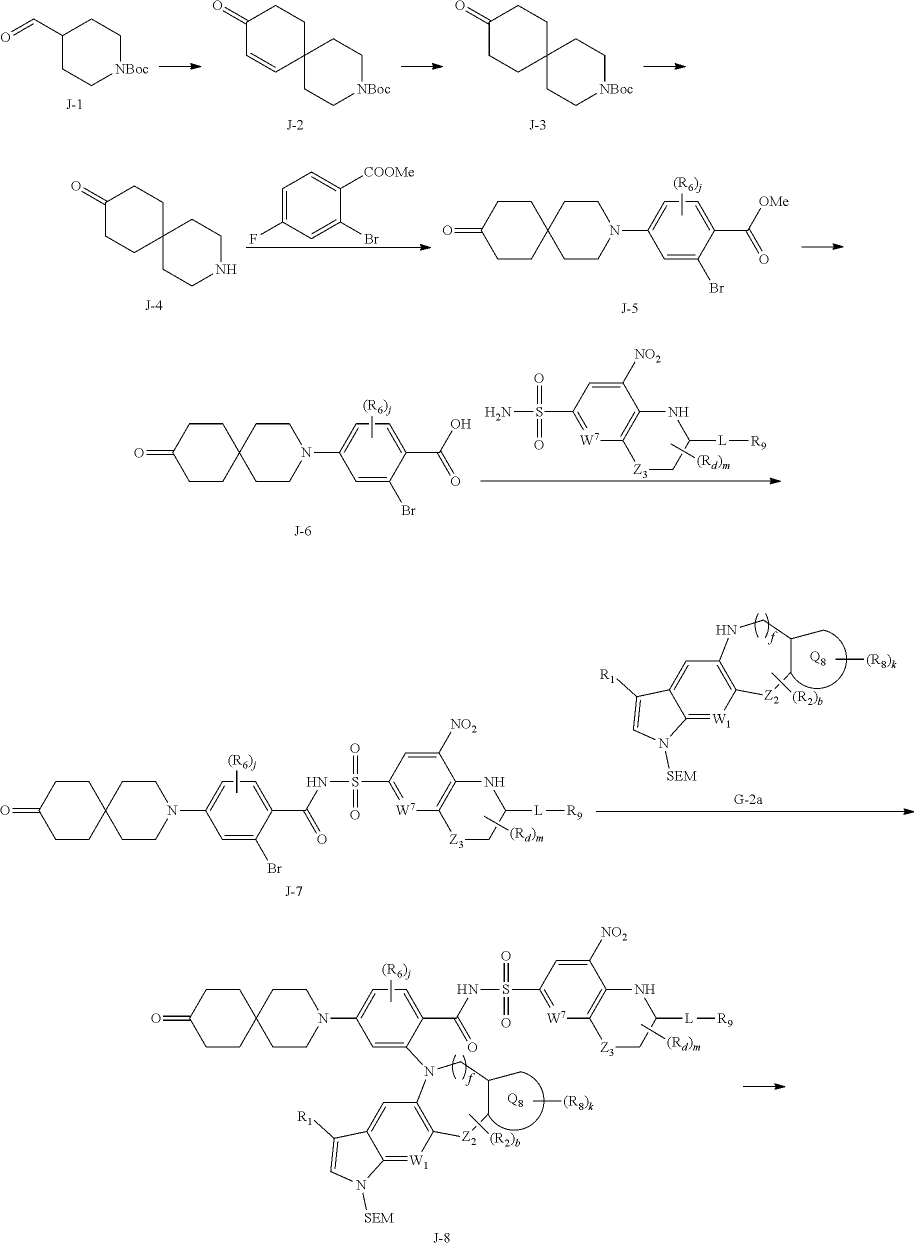

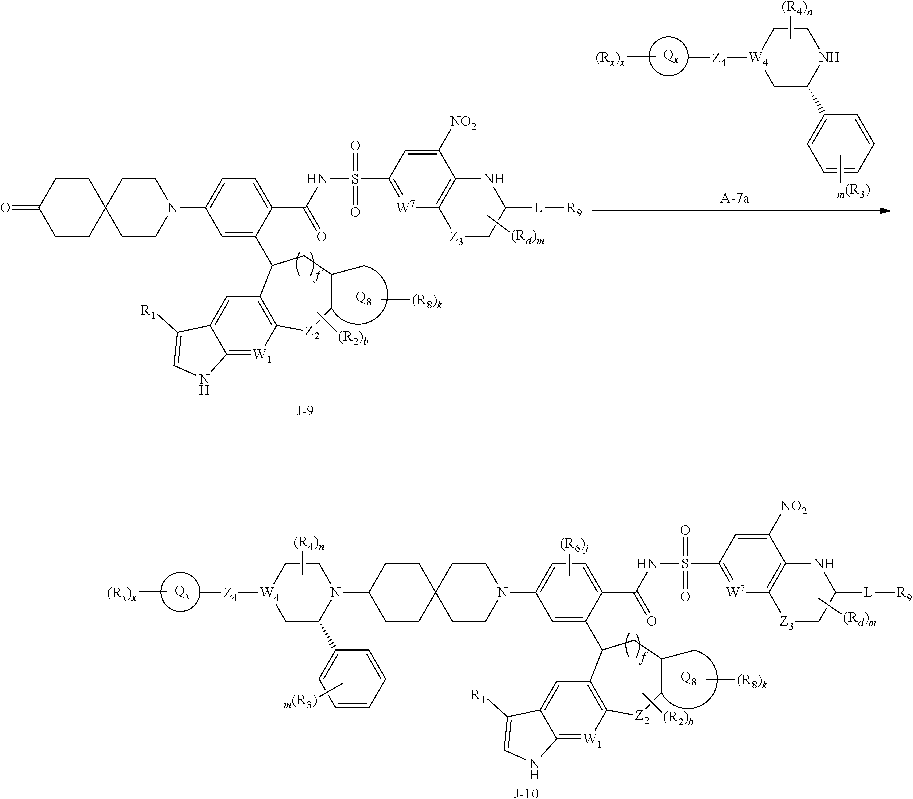

In Scheme J, the starting material J-1 can be converted to J-4 via literature known conditions. The intermediate J-4 can go through a SNAr substitution reaction to afford J-5. After that, the hydrolysis of J-5 can yield J-6, which is further converted to J-7. Next, the coupling reaction of J-7 with G-2a can generate J-8, which is deprotected to provide the target compounds J-9. Finally, the intermediate J-9 can go through a reductive amination reaction with A-7a to provide the target compounds J-10.

Also, the target compounds can be synthesized by alternative methods but not limited to the above procedures.

Similarly, the compounds of

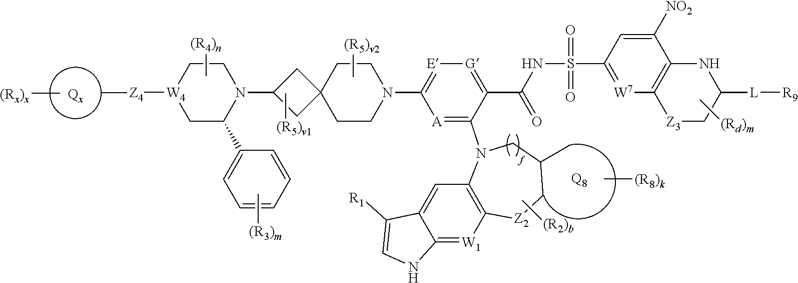

can be prepared by schemes similar to the Scheme A-Scheme J by using appropriate staring materials and intermediates.

Similarly, the compounds of

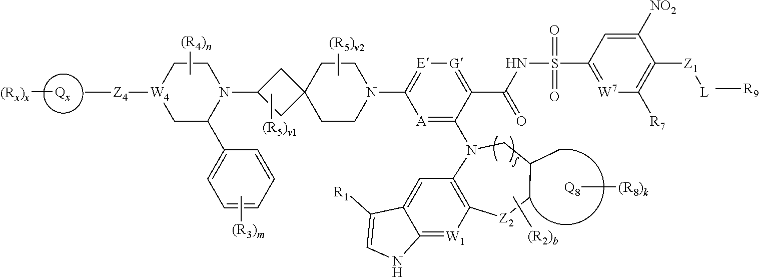

can be prepared by schemes similar to the Scheme A-Scheme J by using appropriate staring materials and intermediates.

Similarly, the compounds of

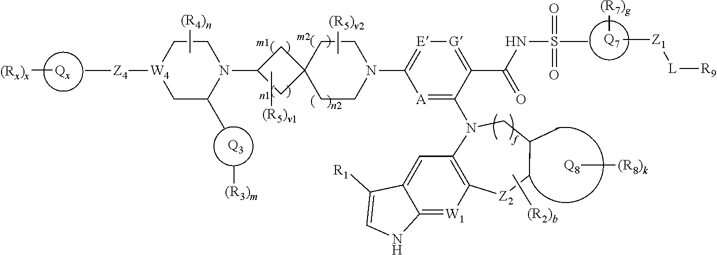

can be prepared by schemes similar to the Scheme A-Scheme J by using appropriate staring materials and intermediates.

Similarly, the compounds of

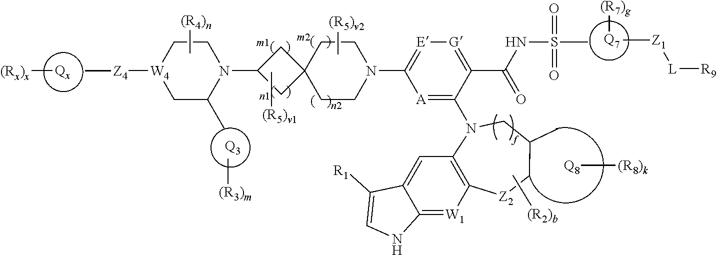

can be prepared by schemes similar to the Scheme A-Scheme J by using appropriate staring materials and intermediates.

Similarly, the compounds of

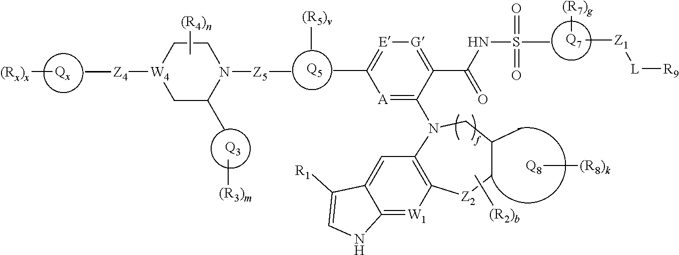

can be prepared by schemes similar to the Scheme A-Scheme J by using appropriate staring materials and intermediates.

Similarly, the compounds of

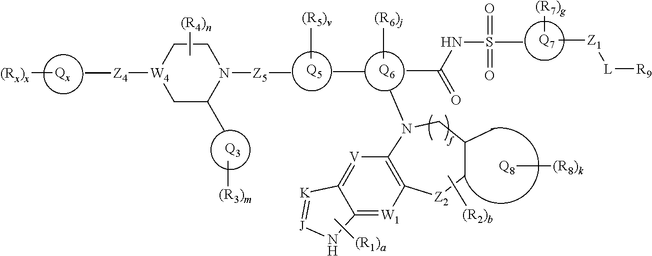

can be prepared by schemes similar to the Scheme A-Scheme J by using appropriate staring materials and intermediates.

Similarly, the compounds of

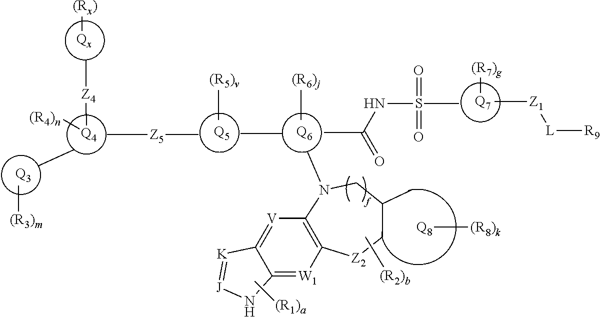

can be prepared by schemes similar to the Scheme A-Scheme J by using appropriate staring materials and intermediates.

Similarly, the compounds of

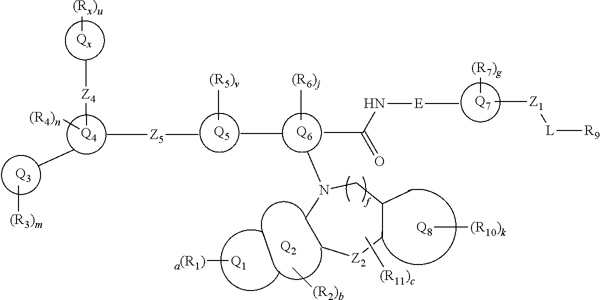

can be prepared by schemes similar to the Scheme A-Scheme J by using appropriate staring materials and intermediates.

The compounds and processes of the present invention will be better understood in connection using the following examples, which are intended as an illustration only and not limiting of the scope of the invention. Various changes and modifications to the disclosed embodiments will be apparent to those skilled in the art and such changes and modifications including, without limitation, those relating to the chemical structures, substituents, derivatives, formulations and/or methods of the invention may be made without departing from the spirit of the invention and the scope of the appended claims.

Where NMR data are presented, ¹H spectra were obtained on XL400 (400 MHz) and are reported as ppm down field from Me₄Si with number of protons, multiplicities, and coupling constants in Hertz indicated parenthetically. Where HPLC data are presented, analyses were performed using an Agilent 1100 system.

Where LC/MS data are presented, analyses were performed using an Applied Biosystems API-100 mass spectrometer and Shimadzu SCL-10A LC column:

### Example INT_2: Preparation of 3-nitro-4-(((tetrahydro-2H-pyran-4-yl)methyl)amino)benzenesulfonamide

To a 500 mL three-neck flask equipped with a mechanical stirrer were charged the 4-chloro-3-nitrobenzenesulfonamide (23.7 g, 100 mmol), DIPEA (12.9 g, 100 mmol), (tetrahydro-2H-pyran-4-yl)methanamine (11.5 g, 100 mmol) and acetonitrile (200 mL). The reaction mixture was adjusted to an internal temperature of 80° C. and agitated for no less than 12 hours. The product solution was cooled down to 40° C. and agitated for no less than 1 hour until precipitation observed. The product slurry was further cooled to 20° C. water (80 mL) was slowly charged over no less than 1 hour, and the mixture cooled to 10° C. and agitated for no less than 2 hours before collected by filtration. The wet cake was washed with 1:1 mix of acetonitrile: water (40 mL). The wet cake was rinsed with water (80 mL) at 40° C. for no less than 1 hour before collected by filtration. The wet cake was rinsed with water (20 mL), and dried at 75° C. under vacuum to give the 3-nitro-4-(((tetrahydro-2H-pyran-4-yl)methyl)amino)benzenesulfonamide (24.5 g, 78%) as an orange solid. ¹H NMR (400 MHz, DMSO-d₆) δ 8.60 (t, J=5.9 Hz, 1H), 8.48 (d, J=2.2 Hz, 1H), 7.84 (dd, J=9.2, 2.0 Hz, 1H), 7.54-7.18 (m, 3H), 3.86 (dd, J=11.3, 3.2 Hz, 2H), 3.35 (s, 2H), 3.27 (t, J=10.9 Hz, 2H), 1.92 (ddd, J=11.2, 7.4, 3.9 Hz, 1H), 1.62 (d, J=11.4 Hz, 2H), 1.27 (qd, J=12.3, 4.4 Hz, 2H).

### Example INT_3: Preparation of 4-[[(4-fluorooxan-4-yl)methyl]amino]-3-nitrobenzene-1-sulfonamide

Into a 50-mL round-bottom flask, were placed (4-fluorooxan-4-yl)methanamine hydrochloride (500 mg, 2.95 mmol, 1.0 eq), 4-fluoro-3-nitrobenzene-1-sulfonamide (650 mg, 2.95 mmol, 1.0 eq), tetrahydrofuran (15 mL), Cs₂CO₃ (2.8 g, 8.59 mmol, 3.0 eq). The resulting solution was stirred for 14 h at 50° C. The reaction mixture was cooled to room temperature. The resulting mixture was filtered and concentrated under vacuum. The residue was applied onto a silica gel column with ethyl acetate/petroleum ether (4:1). Finally, 4-[[(4-fluorooxan-4-yl)methyl]amino]-3-nitrobenzene-1-sulfonamide was obtained as a yellow solid (650 mg, 66%). LC-MS (ESI, m/z) M+1: 334. ¹H NMR (300 MHz, DMSO-d₆): δ 8.58 (t, J=6.3 Hz, 1H), 8.49 (d, J=2.1 Hz, 1H), 7.90-7.80 (m, 1H), 7.44 (d, J=9.3 Hz, 1H), 7.34 (s, 2H), 3.87-3.70 (m, 4H), 3.61-3.50 (m, 2H), 1.95-1.70 (m, 4H).

### Example INT_4: Preparation of (S)-4-((1,4-dioxan-2-yl)methylamino)-3-nitrobenzenesulfonamide

Synthesis of (R)-1-chloro-3-(2-chloroethoxy)propan-2-ol: (R)-2-(chloromethyl)oxirane (500.0 g, 5.4 mol, 1.0 eq) was slowly added to a stirred solution of 2-chloroethanol (870.0, 10.8 mol, 2.0 eq) and BF₃·Et₂O (38.0 g, 27 mmol, 0.05 eq) at 45° C. The reaction mixture was heated on an oil bath for 3 h at 45° C. The reaction mixture was cooled to 25° C. and Diethyl ether (100 mL) was added to this solution. The organic layer was washed with water (2×300 mL), dried over magnesium sulfate, and concentrated to yield a light brown liquid (R)-1-chloro-3-(2-chloroethoxy)propan-2-ol (800.0 g). ¹H NMR (300 MHz, DMSO-d₆) δ 3.85-3.47 (m, 9H).

Synthesis of (R)-2-((2-chloroethoxy)methyl)oxirane: (R)-1-chloro-3-(2-chloroethoxy)propan-2-ol (800.0 g, crude, 4.7 mol, 1.0 eq) was added dropwise to a stirred solution of NaOH (465.0 g, 11.6 mol, 2.5 eq) in water (500 mL) on an ice-bath. The ice-bath was immediately removed after addition of (R)-1-chloro-3-(2-chloroethoxy)propan-2-ol. After stirring 2 h at 25° C., diethyl ether (1.5 L) and water (500 mL) were added. The organic layer was washed with water (50 mL), dried over sodium sulfate, and concentrated to give a light brown liquid (R)-2-((2-chloroethoxy)methyl)oxirane (400.0 g). ¹H NMR (300 MHz, chloroform-d) δ: 3.82-3.52 (m, 5H), 3.40-3.35 (m, 1H), 3.11-3.09 (m, 1H), 2.75-2.73 (m, 2H).

Synthesis of (S)-(1,4-dioxan-2-yl)methanol: (R)-2-((2-chloroethoxy)methyl)oxirane (400.0 g, 2.94 mol, 1.0 eq) was added to a solution of NaOH (294.0 g, 7.35 mol, 2.5 eq) in water (2900 mL) at 25° C. The reaction mixture was heated on an oil bath for 2 h at 90° C. The resulting solution was cooled to 25° C. and adjusted PH value to 5 by HCl (6.0 M). The mixture was concentrated and the residue was distilled (90-95° C., 0.1 kPa) under vacuum pump to give a colorless oil (S)-(1,4-dioxan-2-yl)methanol (110 g, 31.7%). ¹H NMR: (300 MHz, chloroform-d) δ: 3.85-3.42 (m, 9H), 2.15 (bs, 1H).

Synthesis of (R)-(1,4-dioxan-2-yl)methyl methanesulfonate: A mixture of (S)-(1,4-dioxan-2-yl)methanol (50.0 g, 0.42 mol, 1.0 eq), TEA (63.6 g, 0.63 mol, 1.5 eq) and DCM (500 mL) at ice-bath, MsCl (48.1 g, 0.42 mol, 1.0 eq) was added dropwise. And then, the ice-bath removed and the mixture was stirred at 25° C. for 2 hours. The reaction mixture was washed by water (2×50 mL) and the organic phase was dried over sodium sulfate, and concentrated to give a light brown oil (R)-(1,4-dioxan-2-yl)methyl methanesulfonate (71.0 g, 83%). ¹H NMR (300 MHz, chloroform-d₆) δ4.23-4.20 (m, 2H), 3.82-3.56 (m, 6H), 3.50-3.40 (m, 1H), 3.02 (m, 3H).

Synthesis of (S)-(1,4-dioxan-2-yl)methanamine: In 1000 mL autoclave, a solution of (R)-(1,4-dioxan-2-yl)methyl methanesulfonate (70.0 g, 0.36 mol, 1.0 eq) in NH₃·MeOH (7 M, 500 mL) was stirred at 80° C. for 12 hours, the reaction mixture was cooled to 25° C. and concentrated to give a light brown oil (S)-(1,4-dioxan-2-yl)methanamine (30.0 g, 73%). ¹H NMR (300 MHz, DMSO-d₆) δ 8.27 (bs, 2H), 3.82-3.42 (m, 6H), 3.24-3.20 (m, 1H), 2.98-2.62 (m, 2H).

Synthesis of (S)-4-((1,4-dioxan-2-yl)methylamino)-3-nitrobenzenesulfonamide: A mixture of (S)-(1,4-dioxan-2-yl)methanamine (25.0 g, 0.21 mol, 1.0 eq), 4-fluoro-3-nitrobenzenesulfonamide (46.0 g, 0.21 mol, 1.0 eq) and Cs₂CO₃ (137.3 g, 0.42 mol, 2.0 eq) in THE (700 mL) was stirred at 50° C. for 6 hours. LCMS showed material was consumed completely, the reaction mixture was cooled to 25° C. and poured into water (3500 mL). The mixture was filtrated and collected filtrate cake and dried by oven to give a yellow solid (S)-4-((1,4-dioxan-2-yl)methylamino)-3-nitrobenzenesulfonamide (60.0 g, 89.5%). ¹H NMR (300 MHz, DMSO-d₆) δ 8.52-8.47 (m, 2H), 7.86-7.83 (m, 1H), 7.28-7.00 (m, 3H), 3.82-3.29 (m, 9H).

### Example INT_12: Preparation of (11R,15S)-4-[[2-(trimethylsilyl)ethoxy]methyl]-13,16-dioxa-2,4,10-triazatetracyclo[7.7.0.0\hat{ }[3,7].0\hat{ }[11,15]]hexadeca-1(9),2,5,7-tetraene and (11S,15R)-4-[[2-(trimethylsilyl)ethoxy]methyl]-13,16-dioxa-2,4,10-triazatetracyclo[7.7.0.0\hat{ }[3,7].0\hat{ }[11,15]]hexadeca-1(9),2,5,7-tetraene

Synthesis of N-[(trans)(3S,4R)-4-hydroxyoxolan-3-yl]-4-methylbenzenesulfonamide: Into a 250 mL round-bottom flask purged and maintained with an inert atmosphere of nitrogen, were placed 3,6-dioxabicyclo[3.1.0]hexane (5.0 g, 58.1 mmol, 1.0 eq), dioxane (100 mL), p-toluenesulfonamide (11.9 g, 69.7 mmol, 1.2 eq), N-benzyl-N,N-diethylethanaminium chloride (TEBAC) (1.3 g, 5.8 mmol, 0.1 eq), K₂CO₃ (0.8 g, 5.8 mmol, 0.1 eq). The resulting solution was stirred for 3 days at 90° C. The reaction mixture was cooled to 25° C. The solids were filtered out. The resulting mixture was concentrated under vacuum. The crude residue was purified by a flash column (silica gel, ethyl acetate/petroleum ether=2: 1). This resulted in N-[(trans)(3S,4R)-4-hydroxyoxolan-3-yl]-4-methylbenzenesulfonamide (11 g, 25.7%) as white solid. LC-MS (ESI, m/z) M+1: 258.

Synthesis of N-[(trans)(3S,4R)-4-[(5-bromo-1-[[2-(trimethylsilyl)ethoxy]methyl]pyrrolo[2,3-b]pyridin-6-yl)oxy]oxolan-3-yl]-4-methylbenzenesulfonamide: Into a 250 mL round-bottom flask purged and maintained with an inert atmosphere of nitrogen, were placed N-[(trans)(3S,4R)-4-hydroxyoxolan-3-yl]-4-methylbenzenesulfonamide (10.5 g, 14.7 mmol, 1.3 eq, 36%), tetrahydrofuran (100 mL). This was followed by the addition of NaH (2.7 g, 67.7 mmol, 6.0 eq, 60%), in portions at 0° C. The resulting solution was stirred for 30 mins at 0° C. To this was added 5-bromo-6-fluoro-1-[[2-(trimethylsilyl)ethoxy]methyl]pyrrolo[2,3-b]pyridine (3.9 g, 11.2 mmol, 1.0 eq) at 0° C. The resulting solution was stirred for 4 hours at 70° C. The reaction mixture was cooled to 25° C. The reaction was then quenched by the addition of aqueous NH₄Cl (500 mL). The resulting solution was extracted with ethyl acetate (3×300 mL) and the organic layers combined. The resulting mixture was washed with brine (1500 mL). The mixture was dried over anhydrous Na₂SO₄. After filtration, the filtrate was concentrated under reduced pressure. The crude residue was purified by a flash column (silica gel, ethyl acetate/petroleum ether=1: 2) to afford N-[(trans)(3S,4R)-4-[(5-bromo-1-[[2-(trimethylsilyl)ethoxy]methyl]pyrrolo[2,3-b]pyridin-6-yl)oxy]oxolan-3-yl]-4-methylbenzenesulfonamide (3 g, 45.7%) as light yellow oil. ¹H NMR (300 MHz, Chloroform-d) δ 8.04 (s, 1H), 7.78-7.61 (m, 2H), 7.25-7.10 (m, 3H), 6.43 (d, J=3.6 Hz, 1H), 5.67 (d, J=10.8 Hz, 1H), 5.60 (d, J=5.7 Hz, 1H), 5.49 (d, J=10.8 Hz, 1H), 5.40 (dt, J=6.1, 3.1 Hz, 1H), 4.27 (dd, J=10.5, 5.9 Hz, 1H), 4.21-4.03 (m, 1H), 4.03-3.86 (m, 2H), 3.70-3.52 (m, 3H), 2.32 (s, 3H), 0.93 (ddt, J=10.6, 5.5, 2.6 Hz, 2H), −0.02 (s, 9H).

Synthesis of (trans)(11R,15S)-10-(4-methylbenzenesulfonyl)-4-[[2-(trimethylsilyl)ethoxy]methyl]-13,16-dioxa-2,4,10-triazatetracyclo[7.7.0.0\hat{ }[3,7].0\hat{ }[11,15]]hexadeca-1(9),2,5,7-tetraene: Into a 250 mL round-bottom flask purged and maintained with an inert atmosphere of nitrogen, were placed N-[(trans)(3S,4R)-4-[(5-bromo-1-[[2-(trimethylsilyl)ethoxy]methyl]pyrrolo[2,3-b]pyridin-6-yl)oxy]oxolan-3-yl]-4-methylbenzenesulfonamide (3.0 g, 5.1 mmol, 1.0 eq), N,N-dimethylformamide (50 mL), 1,10-phenanthroline (743 mg, 4.1 mmol, 0.8 eq), Cul (785 mg, 4.1 mmol, 0.8 eq), K₂CO₃ (2.1 g, 15.5 mmol, 3.0 eq). The resulting solution was stirred for 2 days at 120° C. The reaction mixture was cooled to 25° C. The resulting solution was diluted with water (500 mL). The resulting solution was extracted with ethyl acetate (3×200 mL) and the organic layers combined. The resulting mixture was washed with brine (1000 mL). The mixture was dried over anhydrous Na₂SO₄. After filtration, the filtrate was concentrated under reduced pressure. The crude residue was purified by a flash column (silica gel, ethyl acetate/petroleum ether=1: 2) to afford (trans)(11R,15S)-10-(4-methylbenzenesulfonyl)-4-[[2-(trimethylsilyl)ethoxy]methyl]-13,16-dioxa-2,4,10-triazatetracyclo[7.7.0.0\hat{ }[3,7].0 \hat{ }[11,15]]hexadeca-1(9),2,5,7-tetraene (2 g, 82.2%) as yellow oil. LC-MS (ESI, m/z) M+1: 502.

Synthesis of (11R,15S)-4-[[2-(trimethylsilyl)ethoxy]methyl]-13,16-dioxa-2,4,10-triazatetracyclo[7.7.0.0\hat{ }[3,7].0 \hat{ }[11,15]]hexadeca-1(9),2,5,7-tetraene and (11S,15R)-4-[[2-(trimethylsilyl)ethoxy]methyl]-13,16-dioxa-2,4,10-triazatetracyclo[7.7.0.0\hat{ }[3,7].0\hat{ }[11,15]]hexadeca-1(9),2,5,7-tetraene: Into a 100 mL round-bottom flask, were placed Mg (2.0 g, 83.9 mmol, 19.8 eq), MeOH (30 mL), (trans)(11R,15S)-10-(4-methylbenzenesulfonyl)-4-[[2-(trimethylsilyl)ethoxy]methyl]-13,16-dioxa-2,4,10-triazatetracyclo[7.7.0.0\hat{ }[3,7].0 \hat{ }[11,15]]hexadeca-1(9),2,5,7-tetraene (2.5 g, 4.2 mmol, 1.0 eq, 85%). The resulting solution was stirred for 2 hours at 60° C. The reaction mixture was cooled to 25° C. The resulting solution was diluted with 300/300 mL of NaHCO₃ and CH₂Cl₂. The solids were filtered out and the organic was separated. The mixture was dried over anhydrous Na₂SO₄. After filtration, the filtrate was concentrated under reduced pressure. The crude residue was purified by a flash column (silica gel, ethyl acetate/petroleum ether=1: 3). The crude product was purified by Chiral-Prep-HPLC using the following conditions: Mobile phase: A: n-Hexane (0.1% DEA) B: EtOH; Flow rate: 20 mL/min; Column: DAICEL CHIRALPAK IA, 250*20 mm, 5 um; Gradient: 12% B in 20 min; 220 nm. This resulted in (11R,15S)-4-[[2-(trimethylsilyl)ethoxy]methyl]-13,16-dioxa-2,4,10-triazatetracyclo[7.7.0.0\hat{ }[3,7].0\hat{ }[11,15]]hexadeca-1(9),2,5,7-tetraene (350 mg, 23.7%) as yellow oil. and (11S,15R)-4-[[2-(trimethylsilyl)ethoxy]methyl]-13,16-dioxa-2,4,10-triazatetracyclo[7.7.0.0\hat{ }[3,7].0\hat{ }[11,15]]hexadeca-1(9),2,5,7-tetraene (400 mg, 27.1%) as yellow oil. Peak 1: LC-MS (ESI, m/z) M+1: 348. Peak 1, T_{R}=0.99 min in CHIRAL-HPLC, Column: CHIRALPAK IA-3, 4.6*50 mm, 3 μm. mobile phase A: n-Hexane (0.1% DEA); mobile phase B: Ethanol, Conc. of Phase B: 50.0%, Column Temp: 25° C. ¹H NMR (300 MHz, Chloroform-d) δ 7.39 (s, 1H), 7.21 (d, J=3.6 Hz, 1H), 6.37 (d, J=3.6 Hz, 1H), 5.57 (s, 2H), 4.60 (dt, J=10.1, 7.6 Hz, 1H), 4.39-4.22 (m, 2H), 3.94 (dd, J=9.9, 7.9 Hz, 1H), 3.86-3.69 (m, 2H), 3.64-3.45 (m, 2H), 0.91 (dd, J=8.8, 7.5 Hz, 2H), −0.04 (s, 9H). Peak 2: LC-MS (ESI, m/z) M+1: 348. Peak 2, T_{R}=1.34 min in CHIRAL-HPLC, Column: CHIRALPAK IA-3, 4.6*50 mm, 3 μm. mobile phase A: n-Hexane (0.1% DEA); mobile phase B: Ethanol, Conc. of Phase B: 50.0%, Column Temp: 25° C. ¹H NMR (300 MHz, Chloroform-d) δ 7.39 (s, 1H), 7.21 (d, J=3.6 Hz, 1H), 6.37 (d, J=3.6 Hz, 1H), 5.57 (s, 2H), 4.60 (dt, J=10.1, 7.6 Hz, 1H), 4.39-4.22 (m, 2H), 3.94 (dd, J=9.9, 7.9 Hz, 1H), 3.86-3.69 (m, 2H), 3.64-3.45 (m, 2H), 0.91 (dd, J=8.8, 7.5 Hz, 2H), 0.04 (s, 9H).

### Example INT_18 Preparation of methyl 2-bromo-4-{2-oxo-7-azaspiro[3.5]nonan-7-yl}benzoate

Synthesis of 7-azaspiro[3.5]nonan-2-one hydrochloride: Into a 500 mL 3-necked round-bottom flask were added tert-butyl 2-oxo-7-azaspiro[3.5]nonane-7-carboxylate (10.0 g, 41.8 mmol, 1.0 eq), Ethyl acetate (20 mL) and 2 HCl in Ethyl acetate (80 mL) at 25° C. The resulting mixture was stirred for 4 hours at 25° C. The resulting mixture was concentrated under reduced pressure. This resulted in 7-azaspiro[3.5]nonan-2-one hydrochloride (7.0 g, 99.4%) as white solid. ¹H NMR (300 MHz, DMSO-d₆) δ 9.25 (s, 2H), 3.00 (dq, J=8.6, 4.7 Hz, 4H), 2.90 (s, 4H), 1.94-1.85 (m, 4H).

Synthesis of methyl 2-bromo-4-{2-oxo-7-azaspiro[3.5]nonan-7-yl}benzoate: Into a 500 mL 3-necked round-bottom flask were added 7-azaspiro[3.5]nonan-2-one hydrochloride (7.3 g, 41.6 mmol, 1.0 eq), DMF (100 mL), Cs₂CO₃ (33.8 g, 103.9 mmol, 2.5 eq) and methyl 2-bromo-4-fluorobenzoate (10.5 g, 45.1 mmol, 1.1 eq) at 25° C. The resulting mixture was stirred for 8 hours at 80° C. The mixture was allowed to cool down to 25° C. The resulting mixture was diluted with water (200 mL). The resulting mixture was extracted with Ethyl acetate (3×200 mL). The combined organic layers were washed with brine (300 mL), dried over anhydrous Na₂SO₄. After filtration, the filtrate was concentrated under reduced pressure. The crude residue was purified by a flash column (silica gel, ethyl acetate/petroleum ether=1:2) to afford methyl 2-bromo-4-{2-oxo-7-azaspiro[3.5]nonan-7-yl}benzoate (6.8 g, 46.5%) as light yellow solid. LC-MS (ESI, m/z) M+1: 352/354. ¹H NMR (300 MHz, DMSO-d₆) δ 7.73 (d, J=8.9 Hz, 1H), 7.19 (d, J=2.5 Hz, 1H), 6.99 (ddt, J=9.0, 2.6, 1.3 Hz, 1H), 3.77 (s, 3H), 3.40-3.33 (m, 4H), 2.85 (s, 4H), 1.78-1.71 (m, 4H).

### Example INT_19 Preparation of 2-bromo-N-{3-nitro-4-[(oxan-4-ylmethyl)amino]benzenesulfonyl}-4-{2-oxo-7-azaspiro[3.5]nonan-7-yl}benzamide

Synthesis of methyl 2-bromo-4-{2-oxo-7-azaspiro[3.5]nonan-7-yl}benzoate: Into a 40 mL vial were added methyl 2-bromo-4-{2-oxo-7-azaspiro[3.5]nonan-7-yl}benzoate (1.0 g, 2.8 mmol, 1.0 eq) and MeOH (5 mL), water (5 mL), NaOH (0.5 g, 11.4 mmol, 4.0 eq) at 25° C. The resulting mixture was stirred for 2 hours at 40° C. The mixture was allowed to cool down to 25° C. The resulting mixture was diluted with water (20 mL). The mixture was acidified to pH=6 with HCl (1.0 M). The resulting mixture was extracted with CH₂Cl₂ (3×20 mL). The combined organic layers were washed with brine (20 mL), dried over anhydrous Na₂SO₄. After filtration, the filtrate was concentrated under reduced pressure. The resulting mixture was concentrated under reduced pressure. This resulted in 2-bromo-4-{2-oxo-7-azaspiro[3.5]nonan-7-yl}benzoic acid (1.0 g, 72.9%) as a light yellow solid. LC-MS (ESI, m/z) M+1: 338/340. ¹H NMR (300 MHz, DMSO-d₆) δ 12.55 (s, 1H), 7.74 (d, J=8.9 Hz, 1H), 7.17 (d, J=2.5 Hz, 1H), 6.98 (dd, J=9.0, 2.5 Hz, 1H), 3.35 (dt, J=9.0, 3.3 Hz, 4H), 2.85 (s, 4H), 1.97-1.49 (m, 4H).

Synthesis of 2-bromo-N-{3-nitro-4-[(oxan-4-ylmethyl)amino]benzenesulfonyl}-4-{2-oxo-7-azaspiro[3.5]nonan-7-yl}benzamide: Into a 40 mL vial were added 2-bromo-4-{2-oxo-7-azaspiro[3.5]nonan-7-yl}benzoic acid (1.0 g, 3.0 mmol, 1.0 eq) and 3-nitro-4-[(oxan-4-ylmethyl)amino]benzenesulfonamide (1.0 g, 3.2 mmol, 1.1 eq), DMAP (0.7 g, 5.9 mmol, 2.0 eq), CH₂Cl₂ (10 mL), EDCl (0.7 g, 3.5 mmol, 1.2 eq) at 25° C. The resulting mixture was stirred for 3 hours at 30° C. The resulting mixture was diluted with water (20 mL). The resulting mixture was extracted with CH₂Cl₂ (3×20 mL). The combined organic layers were washed with brine (20 mL), dried over anhydrous Na₂SO₄. After filtration, the filtrate was concentrated under reduced pressure. The crude residue was purified by a flash column (silica gel, ethyl acetate/petroleum ether=1:1) to afford 2-bromo-N-{3-nitro-4-[(oxan-4-ylmethyl)amino]benzenesulfonyl}-4-{2-oxo-7-azaspiro[3.5]nonan-7-yl}benzamide (600 mg, 31.9%) as yellow solid. LC-MS (ESI, m/z) M+1: 635/637. ¹H NMR (300 MHz, DMSO-d₆) δ 12.25 (s, 1H), 8.68 (t, J=6.1 Hz, 1H), 8.62 (d, J=2.3 Hz, 1H), 7.94 (dd, J=9.3, 2.3 Hz, 1H), 7.33 (dd, J=9.1, 7.5 Hz, 2H), 7.13 (d, J=2.4 Hz, 1H), 6.96 (dd, J=8.8, 2.5 Hz, 1H), 3.90-3.82 (m, 2H), 3.38 (t, J=6.5 Hz, 2H), 3.35-3.22 (m, 5H), 2.84 (s, 4H), 1.92 (dtd, J=10.4, 7.7, 4.0 Hz, 1H), 1.77-1.69 (m, 4H), 1.63 (dd, J=13.1, 3.7 Hz, 2H), 1.28 (qd, J=12.1, 4.4 Hz, 2H).

### Example INT_20 Preparation of N-((3-nitro-4-(((tetrahydro-2H-pyran-4-yl)methyl)amino)phenyl)sulfonyl)-4-(2-oxo-7-azaspiro[3.5]nonan-7-yl)-2-((5aR,8aS)-1-((2-(trimethylsilyl)ethoxy)methyl)-5a,6,8,8a-tetrahydrofuro[3,4-b]pyrrolo[3′,2′:5,6]pyrido[3,2-e][1,4]oxazin-5(1H)-yl)benzamide

Synthesis of N-((3-nitro-4-(((tetrahydro-2H-pyran-4-yl)methyl)amino)phenyl)sulfonyl)-4-(2-oxo-7-azaspiro[3.5]nonan-7-yl)-2-((5aR,8aS)-1-((2-(trimethylsilyl)ethoxy)methyl)-5a,6,8,8a-tetrahydrofuro[3,4-b]pyrrolo[3′,2′:5,6]pyrido[3,2-e][1,4]oxazin-5(1H)-yl)benzamide: Into a 40 mL vial were added 2-bromo-N-{3-nitro-4-[(oxan-4-ylmethyl)amino]benzenesulfonyl}-4-{2-oxo-7-azaspiro[3.5]nonan-7-yl}benzamide (600 mg, 0.9 mmol, 1.0 eq) and (11R,15S)-4-{[2-(trimethylsilyl)ethoxy]methyl}-13,16-dioxa-2,4,10-triazatetracyclo[7.7.0.0\hat{ }{3,7}.0\hat{ }{11,15}]hexadeca-1(9),2,5,7-tetraene (328 mg, 0.9 mmol, 1.0 eq), Cs₂CO₃ (617 mg, 1.9 mmol, 2 eq), copper(I) iodide (36 mg, 0.19 mmol, 0.2 eq), N¹,N²-diphenyloxalamide (45 mg, 0.19 mmol, 0.2 eq), dimethylformamide (6 mL) at 25° C. The resulting mixture was stirred for 2 hours at 100° C. under nitrogen atmosphere. The mixture was allowed to cool down to 25° C. The resulting mixture was diluted with water (20 mL). The resulting mixture was extracted with Ethyl acetate (4×20 mL). The combined organic layers were washed with brine (20 mL), dried over anhydrous Na₂SO₄. After filtration, the filtrate was concentrated under reduced pressure. The crude residue was purified by a flash column (silica gel, ethyl acetate/dichloromethane=10:1) to afford N-((3-nitro-4-(((tetrahydro-2H-pyran-4-yl)methyl)amino)phenyl)sulfonyl)-4-(2-oxo-7-azaspiro[3.5]nonan-7-yl)-2-((5aR,8aS)-1-((2-(trimethylsilyl)ethoxy)methyl)-5a,6,8,8a-tetrahydrofuro[3,4-b]pyrrolo[3′,2′:5,6]pyrido[3,2-e][1,4]oxazin-5(1H)-yl)benzamide (400 mg, 47.0%) as yellow solid. LC-MS (ESI, m/z) M+1: 902. ¹H NMR (300 MHz, DMSO-d₆) δ 12.22 (s, 1H), 8.58 (d, J=6.2 Hz, 1H), 8.29 (d, J=2.3 Hz, 1H), 7.64 (d, J=8.8 Hz, 1H), 7.28 (d, J=3.5 Hz, 1H), 6.98 (dd, J=9.1, 2.4 Hz, 1H), 6.94-6.89 (m, 1H), 6.83 (s, 1H), 6.66 (s, 1H), 6.17-6.08 (m, 1H), 5.47 (dd, J=20.9, 10.2 Hz, 1H), 5.36 (dd, J=10.8, 7.4 Hz, 1H), 4.56 (dq, J=16.9, 8.2 Hz, 1H), 4.34 (dt, J=14.8, 7.5 Hz, 2H), 3.95 (t, J=7.4 Hz, 1H), 3.91-3.79 (m, 4H), 3.61-3.47 (m, 2H), 3.42 (s, 2H), 3.34 (t, J=5.5 Hz, 4H), 2.83 (d, J=5.0 Hz, 4H), 1.93-1.89 (m, 2H), 1.72 (d, J=5.7 Hz, 4H), 1.66 (dd, J=23.9, 11.2 Hz, 2H), 1.28 (dp, J=16.8, 5.4, 4.9 Hz, 3H), 0.91-0.76 (m, 3H), −0.06 (s, 9H).

### Example INT_24 Preparation of 3-nitro-4-([[(1r,4r)-4-hydroxy-4-methylcyclohexyl]methyl]amino)benzenesulfonamide and 3-nitro-4-([[(1s,4s)-4-hydroxy-4-methylcyclohexyl]methyl]amino)benzenesulfonamide

Synthesis of 8-methyl-1,4-dioxaspiro[4.5]decan-8-ol: Into a 2-L 3-necked round-bottom flask purged and maintained with an inert atmosphere of nitrogen, was placed 1,4-dioxaspiro[4.5]decan-8-one (100.0 g, 640.3 mmol, 1.0 eq), THE (500 mL). This was followed by the addition of CH₃MgBr (256 mL, 3.0 M, 1.2 eq) in portions at 0° C. The resulting solution was stirred for 4 hours at 0° C. The reaction was then quenched by the addition of aqueous NH₄Cl. The resulting mixture was concentrated. The residue was dissolved in dichloromethane (500 mL). The resulting mixture was washed with water (3×50 mL). The mixture was dried over anhydrous sodium sulfate. After filtration, the filtrate was concentrated under reduced pressure. This resulted in 8-methyl-1,4-dioxaspiro[4.5]decan-8-ol (110 g, 99.7%) as white oil. ¹H NMR (300 MHz, Chloroform-d) 5: 3.98-3.93 (m, 4H), 2.07-1.52 (m, 8H), 1.27 (s, 3H).

Synthesis of 4-hydroxy-4-methylcyclohexan-1-one: Into a 1-L round-bottom flask, was placed 8-methyl-1,4-dioxaspiro[4.5]decan-8-ol (110.0 g, 639.5 mmol, 1.0 eq), HCl (0.05 M, 500 mL). The resulting solution was stirred for 4 hours at 70° C. The mixture was allowed to cool down to 25° C. The resulting mixture was extracted with Ethyl acetate (3×300 mL). The combined organic layers were washed with brine (300 mL), dried over anhydrous Na₂SO₄. After filtration, the filtrate was concentrated under reduced pressure. The crude residue was purified by a flash column (silica gel, ethyl acetate/petroleum ether=1:2) to afford 4-hydroxy-4-methylcyclohexan-1-one (40 g, 48.9%) as yellow oil. ¹H NMR (300 MHz, Chloroform-d) δ 2.73 (ddd, J=15.3, 12.9, 6.3 Hz, 2H), 2.33-2.18 (m, 2H), 2.06-1.93 (m, 2H), 1.93-1.78 (m, 2H), 1.38 (s, 3H).

Synthesis of 1-methyl-4-(nitromethyl)cyclohex-3-en-1-ol: Into a 500-mL round-bottom flask, was placed 4-hydroxy-4-methylcyclohexan-1-one (20.0 g, 156.0 mmol, 1.0 eq), CH₃NO₂ (100 mL), methyl[2-(methylamino)ethyl]amine (1.4 g, 15.6 mmol, 0.1 eq). The resulting solution was stirred for 6 hours at 100° C. The mixture was allowed to cool down to 25° C. The resulting mixture was concentrated. The crude residue was purified by a flash column (silica gel, ethyl acetate/petroleum ether=1:3) to afford 1-methyl-4-(nitromethyl)cyclohex-3-en-1-ol (21 g, 78.6%) as brown oil. ¹H NMR (300 MHz, Chloroform-d) δ 5.87 (t, J=2.7 Hz, 1H), 4.87 (s, 2H), 2.41-2.11 (m, 4H), 1.85-1.62 (m, 2H), 1.54 (d, J=16.0 Hz, 1H), 1.31 (s, 3H).

Synthesis of 4-(aminomethyl)-1-methylcyclohexan-1-ol: Into a 100-mL pressure tank reactor (20 atm), was placed 1-methyl-4-(nitromethyl)cyclohex-3-en-1-ol (5.0 g, 29.2 mmol, 1.0 eq), i-PrOH (50 mL), Pt/C (1.1 g, 5.8 mmol). To the above, H₂ (gas) was introduced in. The resulting solution was stirred for 20 hours at 30° C. The solids were filtered out. The resulting mixture was concentrated. This resulted in 4-(aminomethyl)-1-methylcyclohexan-1-ol (2.5 g, crude) as black oil.

Synthesis of 3-nitro-4-([[(1r,4r)-4-hydroxy-4-methylcyclohexyl]methyl]amino)benzenesulfonamide and 3-nitro-4-([[(1s,4s)-4-hydroxy-4-methylcyclohexyl]methyl]amino)benzenesulfonamide: Into a 100-mL round-bottom flask, was placed 4-(aminomethyl)-1-methylcyclohexan-1-ol (2.5 g, crude), 4-fluoro-3-nitrobenzenesulfonamide (3.8 g, 17.4 mmol, 1.0 eq), THE (30 mL), TEA (5.3 g, 52.4 mmol, 3.0 eq). The resulting solution was stirred for 14 hours at 25° C. The resulting mixture was concentrated. The residue was applied onto a C18 gel column with (water: CH₃CN=100:0 to 100:30). This resulted in 1 g of 3-nitro-4-([[(1r,4r)-4-hydroxy-4-methylcyclohexyl]methyl]amino)benzenesulfonamide as a yellow solid. ¹H NMR (400 MHz, DMSO-d₆) δ 8.51 (d, J=6.4 Hz, 1H), 8.47 (s, 1H), 7.85 (d, J=9.2 Hz, 1H), 7.36 (s, 2H), 7.27 (dd, J=9.2, 2.4 Hz, 1H), 4.26 (d, J=2.8 Hz, 1H), 3.34 (s, 2H), 1.69 (d, J=13.6 Hz, 3H), 1.55 (d, J=12.8 Hz, 2H), 1.34 (t, J=12.8 Hz, 2H), 1.16 (d, J=12.8 Hz, 1H), 1.10-1.06 (m, 4H). This resulted in 500 mg of 3-nitro-4-([[(1s,4s)-4-hydroxy-4-methylcyclohexyl]methyl]amino)benzenesulfonamide as a yellow solid. ¹H NMR (400 MHz, DMSO-d₆) δ 8.54 (d, J=6.4 Hz, 1H), 8.47 (s, 1H), 7.84 (d, J=9.2 Hz, 1H), 7.34 (s, 2H), 7.26 (d, J=9.2 Hz, 1H), 3.96 (s, 1H), 3.32 (d, J=6.4 Hz, 2H), 3.20-3.11 (m, 1H), 1.75-1.50 (m, 2H), 1.48-1.30 (m, 4H), 1.23 (td, J=13.2, 3.6 Hz, 3H), 1.08 (s, 3H).

### Example INT_25 Preparation of 4-((((1s,4s)-4-methoxy-4-methylcyclohexyl)methyl)amino)-3-nitrobenzenesulfonamide and 4-((((1r,4r)-4-methoxy-4-methylcyclohexyl)methyl)amino)-3-nitrobenzenesulfonamide

Synthesis of methyl 8-methoxy-1,4-dioxaspiro[4.5]decane-8-carboxylate: Into a 250 mL round-bottom, were placed 1,4-dioxaspiro[4.5]decan-8-one (179.1 g, 1.15 mol, 1.0 eq), CHBr₃ (1300 mL). This was followed by the addition of a solution of KOH (516.2 g, 9.2 mol, 8 eq) in MeOH (1800 mL) at −5° C. The resulting solution was stirred for 3 hours at 0° C. and gradually warmed to 25° C. The reaction mixture was concentrated, and then diluted with water (1000 mL) and extracted with Ethyl acetate (600 mL×3). The combined organics were dried over Na₂SO₄, filtered and concentrated in vacuo. The crude residue was purified by a flash column (silica gel, ethyl acetate/petroleum ether=20:1). This resulted in methyl 8-methoxy-1,4-dioxaspiro[4.5]decane-8-carboxylate (83.8 g, 31.7%) as a colorless oil.

Synthesis of (8-methoxy-1,4-dioxaspiro[4.5]decan-8-yl)methanol: Into a 250 mL round-bottom flask purged and maintained with an inert atmosphere of nitrogen, were placed methyl 8-methoxy-1,4-dioxaspiro[4.5]decane-8-carboxylate (83.77 g, 0.36 mol, 1.0 eq), tetrahydrofuran (400 mL). This was followed by the addition of LiAIH₄ (27.6 g, 0.72 mol, 2.0 eq) in portions at 0° C. The resulting solution was stirred for 30 mins at 0° C. The reaction was then quenched by water (84 mL), aqueous NaOH (15%, 84 mL) and water (250 mL) in this order. The resulting solution was filtered, and the filtrate was concentrated in vacuo. This resulted in (8-methoxy-1,4-dioxaspiro[4.5]decan-8-yl)methanol (82.7 g, crude) as a colorless oil, the crude product was used directly to the next step without further purification.

Synthesis of 8-(iodomethyl)-8-methoxy-1,4-dioxaspiro[4.5]decane: Into a 250 mL round-bottom flask purged and maintained with an inert atmosphere of nitrogen were placed (8-methoxy-1,4-dioxaspiro[4.5]decan-8-yl)methanol (82.7 g, 0.41 mol, 1.0 eq), toluene (2500 mL), imidazole (100.2 g, 1.48 mol, 3.6 eq), PPh₃ (376.4 g, 1.435 mol, 3.5 eq). This was followed by the addition of 12 (27.6 g, 0.41 mol, 1.8 eq) in portions at 0° C. The resulting solution was stirred for 2 hours at 90° C. The reaction mixture was cooled to 25° C. and quenched with aqueous Na₂S₂O₃. The organic phase was collected and the aqueous phase was extracted with ethyl acetate (3×500 mL) and the organic layers were combined. The mixture was dried over anhydrous Na₂SO₄. After filtration, the filtrate was concentrated under reduced pressure. The crude residue was purified by a flash column (silica gel, ethyl acetate/petroleum ether=1:20) to afford 8-(iodomethyl)-8-methoxy-1,4-dioxaspiro[4.5]decane (65.6 g, 51.4%) as colorless oil.

Synthesis of 8-methoxy-8-methyl-1,4-dioxaspiro[4.5]decane: Into a 100 mL round-bottom flask, were placed AIBN (6.9 g, 42 mmol, 0.2 eq), toluene (650 mL), 8-(iodomethyl)-8-methoxy-1,4-dioxaspiro[4.5]decane (65.62 g, 0.21 mol, 1.0 eq) and Bu₃SnH (73 g, 0.25 mol, 1.2 eq). The resulting solution was stirred for 2 hours at 90° C. The reaction mixture was quenched with aqueous CsF. The resulting solution was diluted with water (500 mL) and extracted with Ethyl acetate (3×300 mL). The combined organics were dried over anhydrous Na₂SO₄. After filtration, the filtrate was concentrated under reduced pressure. The crude residue was purified by a flash column (silica gel, ethyl acetate/petroleum ether=1:20) to afford 8-methoxy-8-methyl-1,4-dioxaspiro[4.5]decane (20.3 g, 51.9%) as colorless oil. ¹H NMR (500 MHz, chloroform-d) δ 3.93-3.91 (m, 4H), 3.16 (s, 3H), 1.81-1.77 (m, 4H), 1.55-1.49 (m, 4H), 1.12 (s, 3H).

Synthesis of 4-methoxy-4-methylcyclohexan-1-one: Into a 100 mL round-bottom flask, were placed 8-methoxy-8-methyl-1,4-dioxaspiro[4.5]decane (20.3 g, 0.21 mol, 1.0 eq) and acetone (200 mL). This was followed by the addition of HCl (2.0 M, 100 mL) at 25° C. The resulting solution was stirred for 48 hours at 25° C. The reaction mixture was quenched with saturated aqueous NaHCO₃ (200 mL). The resulting solution was diluted with water (100 mL) and extracted with Ethyl acetate (3×100 mL). The combined organics were dried over anhydrous Na₂SO₄. After filtration, the filtrate was concentrated under reduced pressure. The resulted in 4-methoxy-4-methylcyclohexan-1-one (18.99 g, crude) as colorless oil.

Synthesis of 4-methoxy-4-methylcyclohexane-1-carbonitrile: Into a 250 mL round-bottom flask purged and maintained with an inert atmosphere of nitrogen, were placed 4-methoxy-4-methylcyclohexan-1-one (4.5 g, 31.64 mmol, 1.0 eq), DME (60 mL), EtOH (1.5 mL), TosMic (6.17 g, 31.64 mmol, 1.0 eq). This was followed by the addition of t-BuOK (7.11 g, 63.28 mmol, 2.0 eq) in portions at 0° C. The resulting solution was stirred for 2 hours at 50° C. The reaction mixture was cooled to 25° C., and then diluted with MTBE (200 mL). The suspension was filtered and the filtrate was concentrated in vacuo. The crude residue was purified by a flash column (silica gel, ethyl acetate/petroleum ether=1:20) to afford 4-methoxy-4-methylcyclohexane-1-carbonitrile (2.3 g, 47.5%) as colorless oil.

Synthesis of (4-methoxy-4-methylcyclohexyl)methanamine: Into a 250 mL round-bottom flask purged and maintained with an inert atmosphere of nitrogen, was placed 4-methoxy-4-methylcyclohexane-1-carbonitrile (1 g, 6.53 mmol, 1.0 eq), tetrahydrofuran (20 mL). This was followed by the addition of LiAIH₄ (0.97 g, 26.12 mmol, 4.0 eq) in portions at 0° C. The resulting solution was stirred for 2 hours at 25° C. The reaction was then quenched by water (1 mL), aqueous NaOH (15%, 1 mL) and water (3 mL) in this order. The resulting solution was filtered, the filter cake was washed with THE (3×10 mL) and the filtrate was concentrated in vacuo. This resulted in (4-methoxy-4-methylcyclohexyl)methanamine (1 g, a mixture of two diastereoisomers) as a colorless oil, the crude product was used directly to the next step without further purification.

Synthesis of 4-((((1r,4r)-4-methoxy-4-methylcyclohexyl)methyl)amino)-3-nitrobenzenesulfonamide and 4-((((1s,4s)-4-methoxy-4-methylcyclohexyl)methyl)amino)-3-nitrobenzenesulfonamide: Into a 250 mL round-bottom flask purged and maintained with an inert atmosphere of nitrogen, was placed (4-methoxy-4-methylcyclohexyl)methanamine (1 g, 6.36 mmol, a mixture of two diastereoisomers), 4-chloro-3-nitrobenzenesulfonamide (1.81 g, 0.21 mol, 1.2 eq) and DIPEA (3.33 mL, 3 eq). The resulting solution was stirred for 2 hours at 80° C. The resulting solution was concentrated in vacuo. The crude residue was purified by a flash column (silica gel, DCM/MeOH=100:1) to afford 4-((((1r,4r)-4-methoxy-4-methylcyclohexyl)methyl)amino)-3-nitrobenzenesulfonamide (0.36 g, 15.8%). ¹H NMR (500 MHz, DMSO-d₆) δ 8.53 (t, J=5.5 Hz, 1H), 8.46 (d, J=2.0 Hz, 1H), 8.11 (dd, J=9.5 Hz and 2.0 Hz, 1H), 7.30 (s, 2H), 7.25 (d, J=9.5 Hz, 1H), 3.31-3.29 (m, 2H), 3.05 (s, 3H), 1.78-1.76 (m, 2H), 1.62-1.57 (m, 1H), 1.50-1.48 (m, 2H), 1.25-1.19 (m, 4H), 1.04 (s, 3H). 4-((((1s,4s)-4-methoxy-4-methylcyclohexyl)methyl)amino)-3-nitrobenzenesulfonamide(0.12 g, 5.3%) as yellow solid. ¹H NMR (500 MHz, DMSO-d₆) δ 8.51 (t, J=6.0 Hz, 1H), 8.47 (d, J=2.0 Hz, 1H), 8.12 (dd, J=9.0 Hz and 2.0 Hz, 1H), 7.30 (s, 2H), 7.26 (d, J=9.5 Hz, 1H), 3.36 (t, J=6.0 Hz, 2H), 3.09 (s, 3H), 1.71-1.69 (m, 3H), 1.63-1.61 (m, 2H), 1.40-1.34 (m, 2H), 1.21-1.16 (m, 2H), 1.11 (s, 3H).

### Example INT_26 Preparation of (3R,8R)-16-{[2-(trimethylsilyl)ethoxy]methyl}-2,5-dioxa-10,16,18-triazatetracyclo[9.7.0.0\hat{ }{3,8}.0\hat{ }{13,17}]octadeca-1(11),12,14,17-tetraene and (3S,88)-16-{[2-(trimethylsilyl)ethoxy]methyl}-2,5-dioxa-10,16,18-triazatetracyclo[9.7.0.0\hat{ }{3,8}.0\hat{ }{13,17}]octadeca-1(11),12,14,17-tetraene

Synthesis of 4,4-dimethoxyoxan-3-ol: Into a 500 mL round-bottom flask, were placed KOH (10.7 g, 189.8 mmol, 1.9 eq), methanol (200 mL). After that, to the above mixture was added tetrahydro-pyran-4-one (10.0 g, 99.9 mmol, 1.0 eq) in portions over 30 min at 0° C., followed by the addition a solution of I₂ (20.3 g, 79.9 mmol, 0.8 eq) in methanol (400 mL) dropwise over 2 hours at 0° C. The reaction mixture was stirred for 3 hours at 25° C. under nitrogen atmosphere. The resulting mixture was concentrated under vacuum. The residue was dissolved in toluene (100 mL). The solids were filtered off and the filtrate was concentrated under vacuum to give 4,4-dimethoxyoxan-3-ol as a yellow oil (10.0 g, 61.7%).

Synthesis of 3-(benzyloxy)-4,4-dimethoxyoxane: To a stirred solution of 4,4-dimethoxyoxan-3-ol (10.0 g, 61.7 mmol, 1.0 eq) in tetrahydrofuran (200 mL) was added NaH (1.5 g, 61.7 mmol, 1.0 eq) in portions at 0° C. under nitrogen atmosphere. The reaction mixture was stirred for 30 min at 25° C. under nitrogen atmosphere. To the above mixture were added BnBr (10.6 g, 61.7 mmol, 1.0 eq) and tetrabutylammonium iodide (1.1 g, 3.1 mmol, 0.05 eq). The reaction mixture was stirred for 20 hours at 25° C. The resulting mixture was quenched by the addition of aqueous NH₄Cl (100 mL) and extracted with Et₂O (2×100 mL). The combined organic layer was washed with brine (200 mL), dried over anhydrous NaSO₄. After filtration, the filtrate was concentrated under vacuum. The crude residue was applied onto a silica gel column and eluted with ethyl acetate/petroleum ether=1:1 to give 3-(benzyloxy)-4,4-dimethoxyoxane as a colorless oil (8.0 g, 51.4%). ¹H NMR (300 MHz, DMSO-d₆) δ 7.47-7.30 (m, 5H), 4.65 (d, J=11.7 Hz, 1H), 4.50 (d, J=11.7 Hz, 1H), 4.02-3.88 (m, 1H), 3.72-3.64 (m, 1H), 3.50-3.40 (m, 2H), 3.41-3.32 (m, 1H), 3.13 (s, 3H), 3.10 (s, 3H), 1.93-1.56 (m, 2H).

Synthesis of 3-(benzyloxy)oxan-4-one: Into a 500 mL round-bottom flask, were placed 3-(benzyloxy)-4,4-dimethoxyoxane (30.0 g, 118.9 mmol, 1.0 eq), triethylamine (30 mL), water (30 mL) and chloroform (120 mL). The reaction mixture was stirred for 3 hours at 50° C. The mixture was neutralized to pH=7 with saturated aqueous NaHCO₃ and extracted with Et₂O (3×200 mL). The combined organic layers were washed with brine (200 mL), dried over anhydrous Na₂SO₄. After filtration, the filtrate was concentrated under vacuum to give 3-(benzyloxy)oxan-4-one as a colorless oil (21.0 g, 85.6%). ¹H NMR (400 MHz, DMSO-d₆) δ 7.50-7.25 (m, 5H), 4.73 (d, J=11.8 Hz, 1H), 4.51 (d, J=11.8 Hz, 1H), 4.21-4.10 (m, 2H), 4.06 (dddd, J=11.0, 6.8, 2.8, 1.2 Hz, 1H), 3.63 (td, J=11.1, 3.2 Hz, 1H), 3.50-3.38 (m, 1H), 2.71-2.62 (m, 1H), 2.38 (dt, J=14.0, 3.0 Hz, 1H).

Synthesis of 3-(benzyloxy)oxane-4-carbonitrile: Into a 1000 mL 3-necked round-bottom flask, were placed 3-(benzyloxy)oxan-4-one (21.0 g, 101.8 mmol, 1 eq), ethylene glycol dimethyl ether (400 mL), 1-((isocyanomethyl)sulfonyl)-4-methylbenzene (45.7 g, 234.2 mmol, 2.3 eq), tert-Butanol (17.4 g, 234.2 mmol, 2.3 eq). After that, to the above mixture was added t-BuOK (40.0 g, 356.4 mmol, 3.5 eq) in portions over 30 min at 0° C. The reaction mixture was stirred for additional 16 hours at 25° C. The reaction was quenched by the addition of water (100 mL) and extracted with EtOAc (2×200 mL). The combined organic layers were washed with brine (200 mL), dried over anhydrous Na₂SO₄. After filtration, the filtrate was concentrated under vacuum. The crude residue was applied onto a silica gel column and eluted with ethyl acetate/petroleum ether=1:3 to give 3-(benzyloxy)oxane-4-carbonitrile as a colorless oil (5.0 g, 22.6%). GC-MS (ESI, m/z) M+1: 217.

Synthesis of 1-[3-(benzyloxy)oxan-4-yl]methanamine: Into a 50 mL round-bottom flask, were placed 3-(benzyloxy)oxane-4-carbonitrile (1.2 g, 5.5 mmol, 1.0 eq), methanol (20 mL), Raney Ni (50 mg, 0.6 mmol, 0.1 eq). The reaction mixture was stirred for 5 hours at 25° C. under hydrogen atmosphere. The solids were filtered out and the filter cake was washed with methanol (2×10 mL). The filtrate was concentrated under vacuum to give 1-[3-(benzyloxy)oxan-4-yl]methanamine as a colorless oil (1.0 g, 81.8%).

Synthesis of N-{[3-(benzyloxy)oxan-4-yl]methyl}-4-methylbenzenesulfonamide (trans): Into a 50 mL 3-necked round-bottom flask, were placed 1-[3-(benzyloxy)oxan-4-yl]methanamine (1.1 g, 4.9 mmol, 1.0 eq), dichloromethane (20 mL), triethylamine (1.0 g, 9.9 mmol, 2.0 eq). After that, to the above mixture was added 4-methylbenzenesulfonyl chloride (1.1 g, 6.0 mmol, 1.2 eq) in portions at 0° C. The reaction mixture was stirred for 4 hours at 25° C. The resulting mixture was quenched by the addition of water (10 mL) and extracted with CH₂Cl₂ (2×20 mL). The combined organic layer was washed with brine (20 mL), dried over anhydrous Na₂SO₄. After filtration, the filtrate was concentrated under vacuum. The crude residue was applied onto a silica gel column and eluted with ethyl acetate/petroleum ether=1:1 to give N-{[3-(benzyloxy)oxan-4-yl]methyl}-4-methylbenzenesulfonamide (trans) as a white solid (1.1 g, 58.9%). LC-MS (ESI, m/z) M+1: 376.

Synthesis of N-[(3-hydroxyoxan-4-yl)methyl]-4-methylbenzenesulfonamide (trans): Into a 50 mL pressure tank reactor, were placed N-{[3-(benzyloxy)oxan-4-yl]methyl}-4-methylbenzenesulfonamide (1.0 g, 2.7 mmol, 1.0 eq), methanol (20 mL), Pd/C (10%, 300 mg). The mixture was hydrogenated at 60° C. under 30 atm of hydrogen pressure for overnight, filtered through a Celite pad and concentrated under vacuum. The crude residue was applied onto a silica gel column and eluted with ethyl acetate/petroleum ether=1:2 to give N-[(3-hydroxyoxan-4-yl)methyl]-4-methylbenzenesulfonamide (trans) as a colorless oil (570 mg, 75.0%). ¹H NMR (300 MHz, Chloroform-d) δ 7.82-7.73 (m, 2H), 7.39-7.30 (m, 2H), 5.32 (s, 1H), 3.92 (td, J=10.6, 4.7 Hz, 2H), 3.54 (td, J=9.8, 4.9 Hz, 1H), 3.32 (td, J=11.8, 2.2 Hz, 1H), 3.17 (dd, J=13.0, 4.2 Hz, 1H), 3.07 (dd, J=10.9, 10.0 Hz, 1H), 2.95 (dd, J=13.0, 6.4 Hz, 1H), 2.46 (s, 3H), 1.64 (td, J=14.4, 12.7, 4.6 Hz, 2H), 1.49-1.21 (m, 2H).

Synthesis of N-({3-[(5-bromo-1-{[2-(trimethylsilyl)ethoxy]methyl}pyrrolo[2,3-b]pyridin-6-yl)oxy]oxan-4-yl}methyl)-4-methylbenzenesulfonamide (trans): Into a 250 mL round-bottom flask, were placed N-[(3-hydroxyoxan-4-yl)methyl]-4-methylbenzenesulfonamide (trans) (570 mg, 2.0 mmol, 1.0 eq), tetrahydrofuran (20 mL). After that, to the above mixture was added NaH (240 mg, 6.0 mmol, 3.0 eq, 60%) in portions at 0° C. The reaction mixture was stirred for additional 30 min at 0° C., followed by the addition of 5-bromo-6-fluoro-1-{[2-(trimethylsilyl)ethoxy]methyl}pyrrolo[2,3-b]pyridine (690 mg, 2.0 mmol, 1.0 eq) dropwise at 0° C. The resulting mixture was stirred overnight at 60° C. The resulting mixture was quenched by the addition of aqueous NH₄Cl and extracted with EtOAc (2×50 mL). The combined organic layer was washed with brine (200 mL), dried over anhydrous Na₂SO₄. After filtration, the filtrate was concentrated under vacuum. The crude residue was applied onto a silica gel column and eluted with ethyl acetate/petroleum ether=1:1 to give N-({3-[(5-bromo-1-{[2-(trimethylsilyl)ethoxy]methyl}pyrrolo[2,3-b]pyridin-6-yl)oxy]oxan-4-yl}methyl)-4-methylbenzenesulfonamide (trans) as a light yellow oil (460 mg, 37.7%). ¹H NMR (400 MHz, Chloroform-d) δ 8.05 (s, 1H), 7.66-7.58 (m, 2H), 7.20 (d, J=3.6 Hz, 1H), 7.17-7.06 (m, 2H), 6.46-6.37 (m, 1H), 5.55 (q, J=10.8 Hz, 2H), 5.44 (s, 1H), 4.86 (td, J=9.9, 4.7 Hz, 1H), 4.39 (dd, J=10.7, 4.7 Hz, 1H), 4.03-3.94 (m, 1H), 3.53 (td, J=8.5, 7.2 Hz, 2H), 3.46-3.38 (m, 1H), 3.23 (dd, J=10.7, 9.6 Hz, 1H), 3.05 (s, 2H), 2.34 (s, 3H), 2.15-2.03 (m, 1H), 1.86-1.77 (m, 1H), 1.74-1.59 (m, 1H), 0.90 (ddd, J=9.2, 7.1, 1.1 Hz, 2H), −0.05 (s, 9H).

Synthesis of 10-(4-methylbenzenesulfonyl)-16-{[2-(trimethylsilyl)ethoxy]methyl}-2,5-dioxa-10,16,18-triazatetracyclo[9.7.0.0\hat{ }{3,8}.0\hat{ }{13,17}]octadeca-1(11),12,14,17-tetraene (trans): Into a 100 mL round-bottom flask, were placed N-({3-[(5-bromo-1-{[2-(trimethylsilyl)ethoxy]methyl}pyrrolo[2,3-b]pyridin-6-yl)oxy]oxan-4-yl}methyl)-4-methylbenzenesulfonamide (trans) (350 mg, 0.6 mmol, 1.0 eq), Dimethyl sulfoxide (15 mL), K₂CO₃ (238 mg, 1.7 mmol, 3.0 eq), Cul (55 mg, 0.3 mmol, 0.5 eq), picolinic acid (35 mg, 0.3 mmol, 0.5 eq). The reaction mixture was stirred overnight at 135° C. The resulting mixture was quenched by the addition of water (200 mL) and extracted with EtOAc (2×50 mL). The combined organic layer was washed with brine (200 mL), dried over anhydrous Na₂SO₄. After filtration, the filtrate was concentrated under vacuum. The crude residue was applied onto a silica gel column and eluted with ethyl acetate/petroleum ether=3:2 to give 10-(4-methylbenzenesulfonyl)-16-{[2-(trimethylsilyl)ethoxy]methyl}-2,5-dioxa-10,16,18-triazatetracyclo[9.7.0.0\hat{ }{3,8}.0\hat{ }{13,17}]octadeca-1(11),12,14,17-tetraene (trans) as a light yellow oil (230 mg, 75.7%). ¹H NMR (300 MHz, Chloroform-d) δ 8.15 (s, 1H), 7.49-7.40 (m, 2H), 7.35 (d, J=3.6 Hz, 1H), 7.20 (d, J=8.0 Hz, 2H), 6.56 (d, J=3.6 Hz, 1H), 5.69 (d, J=10.5 Hz, 1H), 5.51 (d, J=10.6 Hz, 1H), 4.49 (d, J=15.0 Hz, 1H), 4.19 (d, J=10.0 Hz, 1H), 3.90 (dd, J=11.3, 4.5 Hz, 1H), 3.56 (t, J=8.2 Hz, 2H), 3.38-3.25 (m, 2H), 3.04 (t, J=10.4 Hz, 1H), 2.92 (t, J=13.3 Hz, 1H), 2.40 (s, 3H), 2.01 (s, 2H), 1.67 (d, J=13.4 Hz, 1H), 0.92 (td, J=7.8, 4.4 Hz, 2H), −0.03 (s, 9H).

Synthesis of (3R,8R)-16-{[2-(trimethylsilyl)ethoxy]methyl}-2,5-dioxa-10,16,18-triazatetracyclo[9.7.0.0\hat{ }{3,8}.0\hat{ }{13,17}]octadeca-1(11),12,14,17-tetraene and (3S,88)-16-{[2-(trimethylsilyl)ethoxy]methyl}-2,5-dioxa-10,16,18-triazatetracyclo[9.7.0.0\hat{ }{3,8}.0\hat{ }{13,17}]octadeca-1(11),12,14,17-tetraene: Into a 40 mL vial purged and maintained with an inert atmosphere of nitrogen, was placed Na (60 mg, 2.6 mmol, 6.0 eq), Naphthalene (334 mg, 2.6 mmol, 6.0 eq), ethylene glycol dimethyl ether (10 mL). The mixture was stirred for 40 min at 25° C. until the formation of Na/naphthalene was complete. At the same time, another 100 mL 3-necked round-bottom flask purged and maintained with an inert atmosphere of nitrogen, were placed 10-(4-methylbenzenesulfonyl)-16-{[2-(trimethylsilyl)ethoxy]methyl}-2,5-dioxa-10,16,18-triazatetracyclo[9.7.0.0\hat{ }{3,8}.0\hat{ }{13,17}]octadeca-1(11),12,14,17-tetraene (230 mg, 0.4 mmol, 1.0 eq), tetrahydrofuran (10 mL). This was followed by the addition of the above solution at −78° C. The reaction mixture was stirred for 3 hours at 25° C. The resulting mixture was then quenched by the addition of aqueous NH₄Cl (300 mL) and extracted with EtOAc (3×100 mL). The combined organic layer was washed with brine (300 mL), dried over anhydrous Na₂SO₄. After filtration, the filtrate was concentrated under vacuum. The crude residue was applied onto a silica gel column and eluted with ethyl acetate/petroleum ether=3:2. The crude product was purified by Prep-SFC using the following conditions. Finally, (3R,8R)-16-{[2-(trimethylsilyl)ethoxy]methyl}-2,5-dioxa-10,16,18-triazatetracyclo[9.7.0.0\hat{ }{3,8}.0\hat{ }{13,17}]octadeca-1(11),12,14,17-tetraene was obtained as a brown solid (53 mg, 32.5%) and (3S,8S)-16-{[2-(trimethylsilyl)ethoxy]methyl}-2,5-dioxa-10,16,18-triazatetracyclo[9.7.0.0\hat{ }{3,8}.0\hat{ }{13,17}]octadeca-1(11),12,14,17-tetraene was obtained as a brown solid (72 mg, 44.2%). 5A, T_{R}=3.8 min in CHIRAL-SFC, Column: CHIRAL ART Cellulose-SC, 3*25 cm, 5 um; Mobile Phase A: CO2, Mobile Phase B: MeOH; Flow rate: 80 mL/min; Gradient: 30% B, Wave Length: 220 nm. 5B, T_{R}=5.8 min in CHIRAL-SFC, Column: CHIRAL ART Cellulose-SC, 3*25 cm, 5 um; Mobile Phase A: CO2, Mobile Phase B: MeOH; Flow rate: 80 mL/min; Gradient: 30% B, Wave Length: 220 nm. LC-MS (ESI, m/z) M+1: 376 (peak 1). ¹H NMR (300 MHz, Chloroform-d) δ 7.41 (s, 1H), 7.23 (d, J=3.5 Hz, 1H), 6.36 (d, J=3.5 Hz, 1H), 5.67 (d, J=10.7 Hz, 1H), 5.48 (d, J=10.7 Hz, 1H), 4.41 (dd, J=10.0, 4.1 Hz, 1H), 3.95 (dd, J=11.4, 4.6 Hz, 1H), 3.60-3.56 (m, 1H), 3.56-3.53 (m, 1H), 3.53-3.49 (m, 2H), 3.47 (d, J=1.9 Hz, 1H), 3.31 (dd, J=12.5, 3.5 Hz, 1H), 2.67 (dd, J=12.4, 10.0 Hz, 1H), 2.14 (s, 1H), 1.73-1.61 (m, 1H), 1.42 (qd, J=12.6, 4.7 Hz, 1H), 0.90 (ddd, J=9.2, 7.0, 6.0 Hz, 2H), −0.05 (s, 9H).

### Example INT_27 Preparation of (3S,8S)-16-{[2-(trimethylsilyl)ethoxy]methyl}-2,6-dioxa-10,16,18-triazatetracyclo[9.7.0.0\hat{ }{3,8}.0\hat{ }{13,17}]octadeca-1(11),12,14,17-tetraene and (3R,8R)-16-{[2-(trimethylsilyl)ethoxy]methyl}-2,6-dioxa-10,16,18-triazatetracyclo[9.7.0.0\hat{ }{3,8}.0\hat{ }{13,17}]octadeca-1(11),12,14,17-tetraene

Synthesis of methyl 4-oxooxane-3-carboxylate: Into a 3L 4-necked round-bottom flask, were placed tetrahydro-pyran-4-one (100.0 g, 998.8 mmol, 1.0 eq), dimethyl carbonate (225.0 g, 2497.8 mmol, 2.5 eq), tetrahydrofuran (1000 mL). After that, to the above stirring solution was added NaH (100.0 g, 2500.2 mmol, 2.5 eq, 60%) in portions at 0° C. under nitrogen atmosphere. The reaction mixture was stirred overnight at 45° C. under nitrogen atmosphere. The resulting mixture was quenched by the addition of HCl (1 M, 300 mL) and extracted with EtOAc (2×500 mL). The combined organic layer was washed with brine (1000 mL), dried over anhydrous Na₂SO₄. After filtration, the filtrate was concentrated under vacuum. The crude residue was applied onto a silica gel column and eluted with ethyl acetate/petroleum ether=1:2 to give methyl 4-oxooxane-3-carboxylate as a yellow oil (20.0 g, 12.7%).

Synthesis of methyl 4-hydroxyoxane-3-carboxylate: Into a 1L 3-necked round-bottom flask, were placed methyl 4-oxooxane-3-carboxylate (20.0 g, 126.5 mmol, 1.0 eq), methanol (300 mL). After that, to the above mixture was added NaBH₄ (5.8 g, 153.3 mmol, 1.2 eq) in portions at −78° C. The reaction mixture was stirred for additional 0.5 h at −78° C. The resulting mixture was quenched by the addition of aqueous NH₄Cl and extracted with CH₂Cl₂ (2×200 mL). The combined organic layer was washed with brine (500 mL), dried over anhydrous Na₂SO₄. After filtration, the filtrate was concentrated under vacuum to give methyl 4-hydroxyoxane-3-carboxylate as a colorless oil (9.7 g, 47.9%). ¹H NMR (300 MHz, Chloroform-d) δ 4.31-4.10 (m, 1H), 4.07-3.77 (m, 3H), 3.73 (s, 3H), 3.63 (dt, J=11.5, 4.5 Hz, 1H), 3.04 (s, 1H), 2.89-2.46 (m, 1H), 2.11-1.53 (m, 2H).

Synthesis of methyl 4-[(tert-butyldiphenylsilyl)oxy]oxane-3-carboxylate: Into a 250 mL round-bottom flask, were placed methyl 4-hydroxyoxane-3-carboxylate (6.5 g, 40.6 mmol, 1.0 eq), tetrahydrofuran (100 mL), pyridine (16.0 g, 202.3 mmol, 5.0 eq), AgNO₃ (8.3 g, 48.8 mmol, 1.2 eq), tert-butyl(chloro)diphenylsilane (14.5 g, 52.7 mmol, 1.3 eq). The reaction mixture was stirred for 3 hours at 25° C. under nitrogen atmosphere. The resulting mixture was quenched by the addition of water (300 mL) and extracted with EtOAc (2×100 mL). The combined organic layer was washed with brine (300 mL), dried over anhydrous Na₂SO₄. After filtration, the filtrate was concentrated under vacuum. The crude residue was applied onto a silica gel column and eluted with ethyl acetate/petroleum ether=1:10 to give methyl 4-[(tert-butyldiphenylsilyl)oxy]oxane-3-carboxylate as a colorless oil (9.2 g, 56.9%). ¹H NMR (400 MHz, Chloroform-d) δ 7.80-7.70 (m, 4H), 7.51-7.35 (m, 6H), 4.57 (q, J=3.2 Hz, 1H), 4.12 (dd, J=11.6, 10.5 Hz, 1H), 3.95 (dd, J=11.6, 4.4 Hz, 1H), 3.87 (td, J=11.0, 3.5 Hz, 1H), 3.66-3.55 (m, 1H), 3.51 (s, 3H), 2.65 (ddd, J=10.5, 4.4, 2.8 Hz, 1H), 1.66-1.50 (m, 2H), 1.08 (s, 9H).

Synthesis of 4-[(tert-butyldiphenylsilyl)oxy]oxane-3-carboxylic acid: Into a 250 mL round-bottom flask, were placed methyl 4-[(tert-butyldiphenylsilyl)oxy]oxane-3-carboxylate (9.0 g, 22.6 mmol, 1.0 eq), methanol (30 mL), tetrahydrofuran (20 mL), water (10 mL), LiOH·H₂O (1.9 g, 45.3 mmol, 2.0 eq). The reaction mixture was stirred for 5 hours at 40° C. The resulting mixture was concentrated under vacuum. The residue was acidified to pH=5 with HCl (1 M) and extracted with EtOAc (2×100 mL). The combined organic layers were washed with brine (200 mL), dried over anhydrous Na₂SO₄. After filtration, the filtrate was concentrated under vacuum to give 4-[(tert-butyldiphenylsilyl)oxy]oxane-3-carboxylic acid as a grey solid (8.6 g, 99.0%). LC-MS (ESI, m/z) M+Na⁺: 407.

Synthesis of 4-[(tert-butyldiphenylsilyl)oxy]oxane-3-carboxamide (trans): Into a 250 mL round-bottom flask, were placed 4-[(tert-butyldiphenylsilyl)oxy]oxane-3-carboxylic acid (8.6 g, 22.4 mmol, 1.0 eq), tetrahydrofuran (100 mL), triethylamine (11.3 g, 111.7 mmol, 5.0 eq), NH₄Cl (3.1 g, 58.0 mmol, 2.6 eq), HATU (10.2 g, 26.8 mmol, 1.2 eq). The reaction mixture was stirred overnight at 25° C. The resulting mixture was quenched by the addition of water (300 mL) and extracted with ethyl acetate (2×100 mL). The combined organic layer was washed with brine (200 mL), dried over anhydrous Na₂SO₄. After filtration, the filtrate was concentrated under vacuum. The crude residue was applied onto a silica gel column and eluted with ethyl acetate/petroleum ether=1:1 to give 4-[(tert-butyldiphenylsilyl)oxy]oxane-3-carboxamide (trans) as a white solid (4.5 g, 52.5%). ¹H NMR (300 MHz, Chloroform-d) δ 7.77-7.65 (m, 4H), 7.54-7.35 (m, 6H), 6.20 (s, 1H), 5.74 (s, 1H), 4.24 (td, J=8.0, 4.3 Hz, 1H), 4.08 (dd, J=11.9, 3.9 Hz, 1H), 3.83 (dt, J=11.7, 4.7 Hz, 1H), 3.59 (dd, J=11.9, 8.1 Hz, 1H), 3.32 (ddd, J=11.9, 8.9, 3.4 Hz, 1H), 2.51 (td, J=7.9, 3.9 Hz, 1H), 1.70-1.43 (m, 2H), 1.08 (s, 9H).

Synthesis of 3-(aminomethyl)oxan-4-ol (trans): Into a 100 mL round-bottom flask, were placed 4-[(tert-butyldiphenylsilyl)oxy]oxane-3-carboxamide (trans) (3.0 g, 7.8 mmol, 1.0 eq), tetrahydrofuran (30 mL). After that, to the above mixture was added LAIH₄ (893 mg, 23.5 mmol, 3.0 eq) in portions at 0° C. The reaction mixture was stirred overnight at 25° C. The resulting mixture was quenched by the addition of Na₂SO₄·10H₂O at 0° C. The solids were filtered out and the filtrate was concentrated under vacuum to give 3-(aminomethyl)oxan-4-ol (trans) as a white solid (1.0 g, crude). LC-MS (ESI, m/z) M+1: 132.

Synthesis of N-[(4-hydroxyoxan-3-yl)methyl]-4-methylbenzenesulfonamide (trans): Into a 100 mL 3-necked round-bottom flask, were placed 3-(aminomethyl)oxan-4-ol (trans) (1.0 g, 7.6 mmol, 1.0 eq), dichloromethane (20 mL), triethylamine (1.5 g, 14.8 mmol, 1.9 eq). After that, to the above mixture was added 4-methylbenzenesulfonyl chloride (1.6 g, 8.4 mmol, 1.1 eq) in portions at 0° C. The reaction mixture was stirred for additional 2 hours at 25° C. The resulting mixture was quenched by the addition of water (200 mL) and extracted with CH₂Cl₂ (2×50 mL). The combined organic layer was washed with brine (100 mL), dried over anhydrous Na₂SO₄. After filtration, the filtrate was concentrated under vacuum. The crude residue was applied onto a silica gel column and eluted with ethyl acetate/petroleum ether=2:1 to give N-[(4-hydroxyoxan-3-yl)methyl]-4-methylbenzenesulfonamide (trans) as a colorless oil (2.0 g, 91.9%). ¹H NMR (400 MHz, Chloroform-d) δ 7.79-7.73 (m, 2H), 7.34 (d, J=7.9 Hz, 2H), 4.01-3.92 (m, 1H), 3.90-3.82 (m, 1H), 3.66 (td, J=10.0, 4.6 Hz, 1H), 3.37 (td, J=11.8, 2.3 Hz, 1H), 3.14-3.02 (m, 2H), 2.89 (dd, J=13.1, 6.4 Hz, 1H), 2.46 (s, 3H), 1.90 (ddt, J=12.8, 4.6, 2.3 Hz, 1H), 1.74 (dt, J=10.0, 5.6 Hz, 1H), 1.72-1.56 (m, 1H).

Synthesis of N-({4-[(5-bromo-1-{[2-(trimethylsilyl)ethoxy]methyl}pyrrolo[2,3-b]pyridin-6-yl)oxy]oxan-3-yl}methyl)-4-methylbenzenesulfonamide (trans): Into a 250 mL round-bottom flask, were placed N-[(4-hydroxyoxan-3-yl)methyl]-4-methylbenzenesulfonamide (trans) (2.0 g, 7.0 mmol, 1.0 eq), tetrahydrofuran (30 mL). After that, to the above mixture was added NaH (842 mg, 21.1 mmol, 3.0 eq, 60%) in portions at 0° C. The reaction mixture was stirred for additional 30 min at 0° C., followed by the addition of 5-bromo-6-fluoro-1-{[2-(trimethylsilyl)ethoxy]methyl}pyrrolo[2,3-b]pyridine (2.4 g, 7.0 mmol, 1.0 eq) dropwise at 0° C. The reaction mixture was stirred overnight at 60° C., and then quenched by the addition of aqueous NH₄Cl and extracted with EtOAc (2×50 mL). The combined organic layer was washed with brine (200 mL), dried over anhydrous Na₂SO₄. After filtration, the filtrate was concentrated under vacuum. The crude residue was applied onto a silica gel column and eluted with ethyl acetate/petroleum ether=1:1 to give N-({4-[(5-bromo-1-{[2-(trimethyl silyl)ethoxy]methyl}pyrrolo[2,3-b]pyridin-6-yl)oxy]oxan-3-yl}methyl)-4-methylbenzenesulfonamide (trans) as a light yellow oil (1.6 g, 37.4%). ¹H NMR (300 MHz, Chloroform-d) δ 8.06 (s, 1H), 7.64 (d, J=8.3 Hz, 2H), 7.18 (dd, J=5.9, 2.2 Hz, 3H), 6.42 (d, J=3.6 Hz, 1H), 5.57 (d, J=1.6 Hz, 2H), 5.14 (td, J=8.6, 4.2 Hz, 1H), 4.01 (dd, J=11.7, 4.3 Hz, 2H), 3.65-3.46 (m, 2H), 3.40 (dd, J=11.8, 8.8 Hz, 1H), 3.03 (dt, J=14.8, 8.5 Hz, 2H), 2.37 (s, 3H), 2.33-2.24 (m, 1H), 2.20-2.09 (m, 1H), 1.83-1.75 (m, 2H), 0.96-0.82 (m, 2H), −0.05 (s, 9H).

Synthesis of 10-(4-methylbenzenesulfonyl)-16-{[2-(trimethylsilyl)ethoxy]methyl}-2,6-dioxa-10,16,18-triazatetracyclo[9.7.0.0\hat{ }{3,8}.0\hat{ }{13,17}]octadeca-1(11),12,14,17-tetraene (trans): Into a 100 mL round-bottom flask, were placed N-({4-[(5-bromo-1-{[2-(trimethylsilyl)ethoxy]methyl}pyrrolo[2,3-b]pyridin-6-yl)oxy]oxan-3-yl}methyl)-4-methylbenzenesulfonamide (trans) (900 mg, 1.5 mmol, 1.0 eq), Dimethyl sulfoxide (30 mL), K₂CO₃ (612 mg, 4.4 mmol, 3.0 eq), Cul (140 mg, 0.7 mmol, 0.5 eq), picolinic acid (91 mg, 0.7 mmol, 0.5 eq). The reaction mixture was stirred overnight at 135° C. The resulting mixture was quenched by the addition of water (200 mL) and extracted with EtOAc (2×50 mL). The combined organic layer was washed with brine (200 mL), dried over anhydrous Na₂SO₄. After filtration, the filtrate was concentrated under vacuum. The crude residue was applied onto a silica gel column and eluted with ethyl acetate/petroleum ether=3:2 to give 10-(4-methylbenzenesulfonyl)-16-{[2-(trimethylsilyl)ethoxy]methyl}-2,6-dioxa-10,16,18-triazatetracyclo[9.7.0.0\hat{ }{3,8}.0\hat{ }{13,17}]octadeca-1(11),12,14,17-tetraene (trans) as a yellow oil (548 mg, 70.2%). LC-MS (ESI, m/z) M+1: 530.

Synthesis of (3S,8S)-16-{[2-(trimethylsilyl)ethoxy]methyl}-2,6-dioxa-10,16,18-triazatetracyclo[9.7.0.0\hat{ }{3,8}.0\hat{ }{13,17}]octadeca-1(11),12,14,17-tetraene and (3R,8R)-16-{[2-(trimethylsilyl)ethoxy]methyl}-2,6-dioxa-10,16,18-triazatetracyclo[9.7.0.0\hat{ }{3,8}.0\hat{ }{13,17}]octadeca-1(11),12,14,17-tetraene: Into a 40 mL vial purged and maintained with an inert atmosphere of nitrogen, were placed Na (143 mg, 6.2 mmol, 6.0 eq), Naphthalene (796 mg, 6.2 mmol, 6.0 eq), ethylene glycol dimethyl ether (10 mL). The mixture was stirred for 40 min at 25° C. until the formation of Na/naphthalene was complete. At the same time, another 100 mL 3-necked round-bottom flask purged and maintained with an inert atmosphere of nitrogen, were placed 10-(4-methylbenzenesulfonyl)-16-{[2-(trimethylsilyl)ethoxy]methyl}-2,6-dioxa-10,16,18-triazatetracyclo[9.7.0.0\hat{ }{3,8}.0\hat{ }{13,17}]octadeca-1(11),12,14,17-tetraene (trans) (548 mg, 1.0 mmol, 1.0 eq), tetrahydrofuran (10 mL). This was followed by the addition of the above solution at −78° C. The resulting solution was stirred for 3 hours at 25° C. The reaction was then quenched by the addition of aqueous NH₄Cl (300 mL) and extracted with EtOAc (3×100 mL). The combined organic layer was washed with brine (300 mL), dried over anhydrous Na₂SO₄. After filtration, the filtrate was concentrated under vacuum. The crude residue was applied onto a silica gel column and eluted with ethyl acetate/petroleum ether=3:2. The crude product was purified by Prep-SFC using the following conditions. Finally, (3S,8S)-16-{[2-(trimethylsilyl)ethoxy]methyl}-2,6-dioxa-10,16,18-triazatetracyclo[9.7.0.0\hat{ }{3,8}.0\hat{ }{13,17}]octadeca-1(11),12,14,17-tetraene was obtained as a brown oil (100 mg, 25.7%) and (3R,8R)-16-{[2-(trimethylsilyl)ethoxy]methyl}-2,6-dioxa-10,16,18-triazatetracyclo[9.7.0.0\hat{ }{3,8}.0\hat{ }{13,17}]octadeca-1(11),12,14,17-tetraene was obtained as a brown oil (80 mg, 20.6%). 11A, T_{R}=4.4 min in CHIRAL-SFC, Column: CHIRAL ART Cellulose-SC, 3*25 cm, 5 um; Mobile Phase A: CO2, Mobile Phase B: MeOH; Flow rate: 80 mL/min; Gradient: 30% B, Wave Length: 220 nm. 11B, T_{R}=6.8 min in CHIRAL-SFC, Column: CHIRAL ART Cellulose-SC, 3*25 cm, 5 um; Mobile Phase A: CO2, Mobile Phase B: MeOH; Flow rate: 80 mL/min; Gradient: 30% B, Wave Length: 220 nm. LC-MS (ESI, m/z) M+1: 376. ¹H NMR (300 MHz, Chloroform-d) δ 7.40 (s, 1H), 7.22 (d, J=3.5 Hz, 1H), 6.35 (d, J=3.5 Hz, 1H), 5.66 (d, J=10.7 Hz, 1H), 5.49 (d, J=10.7 Hz, 1H), 4.11 (dd, J=11.7, 4.8 Hz, 1H), 3.92 (dd, J=11.5, 4.4 Hz, 1H), 3.80-3.73 (m, 1H), 3.63-3.48 (m, 2H), 3.41 (td, J=12.3, 2.2 Hz, 1H), 3.27 (dd, J=12.5, 4.0 Hz, 1H), 3.07 (t, J=11.4 Hz, 1H), 2.58-2.49 (m, 1H), 2.36-2.16 (m, 2H), 2.06 (tdd, J=12.8, 11.2, 5.0 Hz, 1H), 0.90 (dp, J=9.8, 7.1 Hz, 2H), −0.06 (s, 9H).

### Example INT_28 Preparation of (6aR,9aS)-1-((2-(trimethylsilyl)ethoxy)methyl)-5,6,6a,7,9,9a-hexahydro-1H-furo[3,4-f]pyrrolo[3′,2′:5,6]pyrido[2,3-b][1,4]oxazepine and (6aS,9aR)-1-((2-(trimethylsilyl)ethoxy)methyl)-5,6,6a,7,9,9a-hexahydro-1H-furo[3,4-f]pyrrolo[3′,2′:5,6]pyrido[2,3-b][1,4]oxazepine

Synthesis of (3S,4R)-4-hydroxyoxolane-3-carbonitrile (racemate): Into a 500 mL 3-necked round-bottom flask were added 3,6-dioxabicyclo[3.1.0]hexane (10.0 g, 116.2 mmol, 1.0 eq) and toluene (100 mL) at 25° C. After that, to the above mixture was added cyanodiethylaluminum (197.47 mL, 197.469 mmol, 1.7 eq) dropwise over 30 min at 25° C. The reaction mixture was stirred for 16 hours at 50° C. The resulting mixture was concentrated under vacuum to give (3S,4R)-4-hydroxyoxolane-3-carbonitrile (racemate) as a colorless oil (6.0 g, crude). GC-MS (ESI, m/z) M+1: 114.

Synthesis of (3R,4S)-4-(aminomethyl)oxolan-3-ol (racemate): Into a 250 mL round-bottom flask were added (3S,4R)-4-hydroxyoxolane-3-carbonitrile (6.0 g, crude, 53.0 mmol, 1.0 eq), MeOH (100 mL) and Raney Ni (0.2 g, 2.7 mmol, 0.05 eq) at 25° C. The resulting mixture was stirred for 3 hours at 25° C. under hydrogen atmosphere. The resulting mixture was filtered, the filter cake was washed with MeOH (2×50 mL). After filtration, the filtrate was concentrated under vacuum to give (3R,4S)-4-(aminomethyl)oxolan-3-ol (racemate) as a colorless oil (4.0 g, crude).

Synthesis of N-{[(3S,4R)-4-hydroxyoxolan-3-yl]methyl}-4-methylbenzenesulfonamide (racemate): Into a 250 mL 3-necked round-bottom flask were added (3R,4S)-4-(aminomethyl)oxolan-3-ol (4.5 g, 38.4 mmol, 1.0 eq), DCM (100 mL) and TEA (9.7 g, 96.0 mmol, 2.5 eq) at 25° C. After that, to the above mixture was added p-toluenesulfonyl chloride (7.3 g, 38.4 mmol, 1.0 eq) in portions over 10 min at 0° C. The reaction mixture was stirred for 4 hours at 25° C. The resulting mixture was quenched by the addition of water (10 mL) at 0° C. The resulting mixture was extracted with CH₂Cl₂ (2×20 mL). The combined organic layers were washed with brine (20 mL), dried over anhydrous Na₂SO₄. After filtration, the filtrate was concentrated under vacuum. The crude residue was applied onto a silica gel column and eluted with ethyl acetate/petroleum ether=1:1 to give N-{[(3S,4R)-4-hydroxyoxolan-3-yl]methyl}-4-methylbenzenesulfonamide (racemate) as a white solid (4.0 g, 38.4%). LC-MS (ESI, m/z) M+1: 272.

Synthesis of N-(((3S,4R)-4-((5-bromo-1-((2-(trimethylsilyl)ethoxy)methyl)-1H-pyrrolo[2,3-b]pyridin-6-yl)oxy)tetrahydrofuran-3-yl)methyl)-4-methylbenzenesulfonamide (trans): Into a 250 mL round-bottom flask, were placed N-(((3S,4R)-4-hydroxytetrahydrofuran-3-yl)methyl)-4-methylbenzenesulfonamide (2.0 g, 7.0 mmol, 1.0 eq), tetrahydrofuran (30 mL). After that, to the above mixture was added NaH (842 mg, 21.1 mmol, 3.0 eq, 60%) in portions at 0° C. The reaction mixture was stirred for additional 30 min at 0° C. To the above mixture was added 5-bromo-6-fluoro-1-{[2-(trimethylsilyl)ethoxy]methyl}pyrrolo[2,3-b]pyridine (2.4 g, 7.0 mmol, 1.0 eq) dropwise at 0° C. The reaction mixture was stirred overnight at 60° C. The resulting mixture was quenched by the addition of aqueous NH₄Cl and extracted with EtOAc (2×50 mL). The combined organic layer was washed with brine (200 mL), dried over anhydrous Na₂SO₄. After filtration, the filtrate was concentrated under vacuum. The crude residue was applied onto a silica gel column and eluted with ethyl acetate/petroleum ether=1:1 to give N-(((3S,4R)-4-((5-bromo-1-((2-(trimethylsilyl)ethoxy)methyl)-1H-pyrrolo[2,3-b]pyridin-6-yl)oxy)tetrahydrofuran-3-yl)methyl)-4-methylbenzenesulfonamide (trans) as a light yellow oil (1.6 g, 37.4%). LC-MS (ESI, m/z) M+1: 596/598.

Synthesis of (6aS,9aR)-5-tosyl-1-((2-(trimethylsilyl)ethoxy)methyl)-5,6,6a,7,9,9a-hexahydro-1H-furo[3,4-f]pyrrolo[3′,2′:5,6]pyrido[2,3-b][1,4]oxazepine (trans): Into a 100 mL round-bottom flask, were placed N-(((3S,4R)-4-((5-bromo-1-((2-(trimethylsilyl)ethoxy)methyl)-1H-pyrrolo[2,3-b]pyridin-6-yl)oxy)tetrahydrofuran-3-yl)methyl)-4-methylbenzenesulfonamide (trans) (900 mg, 1.5 mmol, 1.0 eq), Dimethyl sulfoxide (30 mL), K₂CO₃ (612 mg, 4.4 mmol, 3.0 eq), Cul (140 mg, 0.7 mmol, 0.5 eq), picolinic acid (91 mg, 0.7 mmol, 0.5 eq). The reaction mixture was stirred overnight at 135° C. The resulting mixture was quenched by the addition of water (200 mL) and extracted with EtOAc (2×50 mL). The combined organic layer was washed with brine (200 mL), dried over anhydrous Na₂SO₄. After filtration, the filtrate was concentrated under vacuum. The crude residue was applied onto a silica gel column and eluted with ethyl acetate/petroleum ether=3:2 to give (6aS,9aR)-5-tosyl-1-((2-(trimethylsilyl)ethoxy)methyl)-5,6,6a,7,9,9a-hexahydro-1H-furo[3,4-f]pyrrolo[3′,2′:5,6]pyrido[2,3-b][1,4]oxazepine (trans) as a yellow oil (548 mg, 70.2%). LC-MS (ESI, m/z) M+1: 516.

Synthesis of (6aS,9aR)-1-((2-(trimethylsilyl)ethoxy)methyl)-5,6,6a,7,9,9a-hexahydro-1H-furo[3,4-f]pyrrolo[3′,2′:5,6]pyrido[2,3-b][1,4]oxazepine (trans): Into a 40 mL vial purged and maintained with an inert atmosphere of nitrogen, were placed Na (143 mg, 6.2 mmol, 6.0 eq), Naphthalene (796 mg, 6.2 mmol, 6.0 eq), ethylene glycol dimethyl ether (10 mL). The reaction mixture was stirred for 40 min at 25° C. until the formation of Na/naphthalene was complete. At the same time, another 100 mL 3-necked round-bottom flask purged and maintained with an inert atmosphere of nitrogen, were placed (6aS,9aR)-5-tosyl-1-((2-(trimethylsilyl)ethoxy)methyl)-5,6,6a,7,9,9a-hexahydro-1H-furo[3,4-f]pyrrolo[3′,2′:5,6]pyrido[2,3-b][1,4]oxazepine (trans) (548 mg, 1.0 mmol, 1.0 eq) and tetrahydrofuran (10 mL). This was followed by the addition of the above solution at −78° C. The resulting solution was stirred for 3 hours at 25° C. The reaction was then quenched by the addition of aqueous NH₄Cl (300 mL) and extracted with EtOAc (3×100 mL). The combined organic layer was washed with brine (300 mL), dried over anhydrous Na₂SO₄. After filtration, the filtrate was concentrated under vacuum. The crude residue was applied onto a silica gel column and eluted with ethyl acetate/petroleum ether=3:2. The crude product was purified by Prep-SFC to afford (6aS,9aR)-1-((2-(trimethylsilyl)ethoxy)methyl)-5,6,6a,7,9,9a-hexahydro-1H-furo[3,4-f]pyrrolo[3′,2′:5,6]pyrido[2,3-b][1,4]oxazepine (trans) as a brown oil (200 mg, 50.7%). LC-MS (ESI, m/z) M+1: 362.

Synthesis of (6aR,9aS)-1-((2-(trimethylsilyl)ethoxy)methyl)-5,6,6a,7,9,9a-hexahydro-1H-furo[3,4-f]pyrrolo[3′,2′:5,6]pyrido[2,3-b][1,4]oxazepine and (6aS,9aR)-1-((2-(trimethylsilyl)ethoxy)methyl)-5,6,6a,7,9,9a-hexahydro-1H-furo[3,4-f]pyrrolo[3′,2′:5,6]pyrido[2,3-b][1,4]oxazepane: 200 mg of (6aS,9aR)-1-((2-(trimethylsilyl)ethoxy)methyl)-5,6,6a,7,9,9a-hexahydro-1H-furo[3,4-f]pyrrolo[3′,2′:5,6]pyrido[2,3-b][1,4]oxazepine (trans) was purified by Chiral-Prep-HPLC using the following conditions: Column: CHIRALPAK 1H, 3*25 cm, 5 μm; mobile phase A: CO₂; mobile phase B: MeOH—Preparative; Flow rate: 80 mL/min; Gradient: isocratic 30% B; Wave Length: 220 nm. Finally, 90 mg of (6aR,9aS)-1-((2-(trimethylsilyl)ethoxy)methyl)-5,6,6a,7,9,9a-hexahydro-1H-furo[3,4-f]pyrrolo[3′,2′:5,6]pyrido[2,3-b][1,4]oxazepine was obtained as a yellow oil and 96 mg of (6aS,9aR)-1-((2-(trimethylsilyl)ethoxy)methyl)-5,6,6a,7,9,9a-hexahydro-1H-furo[3,4-f]pyrrolo[3′,2′:5,6]pyrido[2,3-b][1,4]oxazepine was obtained as a yellow oil. LC-MS (ESI, m/z) M+1: 362. 6A, T_{R}=1.342 min in CHIRAL-HPLC, Column: CHIRALPAK IH, 3*25 cm, 5 μm. mobile phase A: CO₂; mobile phase B: MeOH—Preparative, Pump Mode: Low pressure gradient, Conc. of Pump B: 30.0%, Oven Temperature: 35° C. LC-MS (ESI, m/z) M+1: 362. 6B, T_{R}=1.559 min in CHIRAL-HPLC, Column: CHIRALPAK IH, 3*25 cm, 5 μm. mobile phase A: CO₂; mobile phase B: MeOH—Preparative, Pump Mode: Low pressure gradient, Conc. of Pump B: 30.0%, Oven Temperature: 35° C.

### Example INT_29 Preparation of (3R,8S)-15-{[2-(trimethylsilyl)ethoxy]methyl}-2,5-dioxa-9,15,17-triazatetracyclo[8.7.0.0\hat{ }{3,8}.0\hat{ }{12,16}]heptadeca-1(10),11,13,16-tetraene

Synthesis of N-((3R,4S)-4-hydroxytetrahydro-2H-pyran-3-yl)-4-methylbenzenesulfonamide: Into a 100 mL 3-necked round-bottom flask, were placed (3R,4S)-3-aminotetrahydro-2H-pyran-4-ol (1.0 g, 8.5 mmol, 1.0 eq), dichloromethane (20 mL), triethylamine (2.1 g, 21.3 mmol, 2.5 eq). After that, to the above mixture was added 4-methylbenzenesulfonyl chloride (1.6 g, 9.4 mmol, 1.1 eq) in portions at 0° C. The reaction mixture was stirred for 4 hours at 25° C. under nitrogen atmosphere. The resulting mixture was quenched by the addition of water (100 mL) and extracted with CH₂Cl₂ (2×20 mL). The combined organic layer was washed with brine (20 mL), dried over anhydrous Na₂SO₄. After filtration, the filtrate was concentrated under vacuum. The crude residue was applied onto a silica gel column and eluted with ethyl acetate/petroleum ether=1:1 to give N-((3R,4S)-4-hydroxytetrahydro-2H-pyran-3-yl)-4-methylbenzenesulfonamide as a white solid (1.5 g, 64.7%). LC-MS (ESI, m/z) M+1: 272.

Synthesis of N-[(3R,4S)-3-[(5-bromo-1-{[2-(trimethylsilyl)ethoxy]methyl}pyrrolo[2,3-b]pyridin-6-yl)oxy]oxan-4-yl]-4-methylbenzenesulfonamide: Into a 100 mL round-bottom flask, were placed N-[(3R,4S)-3-hydroxyoxan-4-yl]-4-methylbenzenesulfonamide (800 mg, 2.9 mmol, 1.0 eq), tetrahydrofuran (10 mL). After that, to the above mixture was added NaH (354 mg, 8.8 mmol, 3.0 eq, 60%) in portions at 0° C. The reaction mixture was stirred for additional 30 min at 0° C. To the above mixture was added 5-bromo-6-fluoro-1-{[2-(trimethylsilyl)ethoxy]methyl}pyrrolo[2,3-b]pyridine (1.0 g, 2.9 mmol, 1.0 eq) dropwise at 0° C. The reaction mixture was stirred overnight at 50° C. The resulting mixture was quenched by the addition of aqueous NH₄Cl and extracted with EtOAc (2×50 mL). The combined organic layer was washed with brine (200 mL), dried over anhydrous Na₂SO₄. After filtration, the filtrate was concentrated under vacuum. The crude residue was applied onto a silica gel column and eluted with ethyl acetate/petroleum ether=1:2 to give N-[(3R,4S)-3-[(5-bromo-1-{[2-(trimethylsilyl)ethoxy]methyl}pyrrolo[2,3-b]pyridin-6-yl)oxy]oxan-4-yl]-4-methylbenzenesulfonamide as a white solid (400 mg, 22.7%). LC-MS (ESI, m/z) M+1: 596/598. ¹H NMR (300 MHz, Chloroform-d) δ 7.99 (s, 1H), 7.39-7.32 (m, 2H), 7.22 (d, J=3.6 Hz, 1H), 7.04 (d, J=8.0 Hz, 2H), 6.44 (d, J=3.6 Hz, 1H), 5.67-5.50 (m, 2H), 4.91 (td, J=9.8, 5.1 Hz, 1H), 4.32-4.07 (m, 1H), 3.95 (dd, J=12.0, 4.5 Hz, 1H), 3.73-3.51 (m, 2H), 3.40 (ddd, J=21.3, 11.7, 10.0 Hz, 3H), 2.37 (s, 3H), 2.05-1.85 (m, 1H), 1.33-1.22 (m, 1H), 1.08-0.93 (m, 2H), −0.00 (s, 9H).

Synthesis of (3R,8S)-9-(4-methylbenzenesulfonyl)-15-{[2-(trimethylsilyl)ethoxy]methyl}-2,5-dioxa-9,15,17-triazatetracyclo[8.7.0.0\hat{ }{3,8}.0\hat{ }{12,16}]heptadeca-1(10),11,13,16-tetraene: Into a 250 mL round-bottom flask, were placed N-[(3R,4S)-3-[(5-bromo-1-{[2-(trimethylsilyl)ethoxy]methyl}pyrrolo[2,3-b]pyridin-6-yl)oxy]oxan-4-yl]-4-methylbenzenesulfonamide (400 mg, 0.7 mmol, 1.0 eq), N,N-dimethylformamide (5 mL), K₂CO₃ (278 mg, 2.0 mmol, 3.0 eq), Cul (77 mg, 0.4 mmol, 0.6 eq), 1,10-phenanthroline (72 mg, 0.4 mmol, 0.6 eq). The reaction mixture was stirred overnight at 120° C. under nitrogen atmosphere. The resulting mixture was quenched by the addition of water (400 mL) and extracted with EtOAc (2×200 mL). The combined organic layer was washed with brine (500 mL), dried over anhydrous Na₂SO₄. After filtration, the filtrate was concentrated under vacuum. The crude residue was applied onto a silica gel column and eluted with ethyl acetate/petroleum ether=2:3 to give (3R,8S)-9-(4-methylbenzenesulfonyl)-15-{[2-(trimethylsilyl)ethoxy]methyl}-2,5-dioxa-9,15,17-triazatetracyclo[8.7.0.0\hat{ }{3,8}.0\hat{ }{12,16}]heptadeca-1(10),11,13,16-tetraene as a colorless oil (300 mg, 86.7%). LC-MS (ESI, m/z) M+1: 516. ¹H NMR (400 MHz, Chloroform-d) δ 8.44 (s, 1H), 7.29-7.23 (m, 3H), 7.13 (d, J=8.2 Hz, 2H), 6.57 (d, J=3.6 Hz, 1H), 5.55 (s, 2H), 4.22 (dd, J=10.9, 4.6 Hz, 1H), 4.06 (dd, J=11.9, 4.4 Hz, 1H), 3.63 (dd, J=10.0, 4.5 Hz, 1H), 3.58-3.46 (m, 4H), 3.31 (t, J=10.3 Hz, 1H), 2.72-2.66 (m, 1H), 2.36 (s, 3H), 2.12 (td, J=12.6, 4.6 Hz, 1H), 1.31-1.23 (m, 2H), −0.04 (s, 9H).

Synthesis of (3R,8S)-15-{[2-(trimethylsilyl)ethoxy]methyl}-2,5-dioxa-9,15,17-triazatetracyclo[8.7.0.0\hat{ }{3,8}.0\hat{ }{12,16}]heptadeca-1(10),11,13,16-tetraene: Into a 40 mL vial purged and maintained with an inert atmosphere of nitrogen, were placed Na (93 mg, 4.0 mmol, 7.0 eq), naphthalene (521 mg, 4.0 mmol, 7.0 eq), ethylene glycol dimethyl ether (3 mL). The mixture was stirred for 40 minutes at 25° C. until the formation of Na/naphthalene was complete. At the same time, another 250 mL 3-necked round-bottom flask purged and maintained with an inert atmosphere of nitrogen, were placed (3R,8S)-9-(4-methylbenzenesulfonyl)-15-{[2-(trimethylsilyl)ethoxy]methyl}-2,5-dioxa-9,15,17-triazatetracyclo[8.7.0.0\hat{ }{3,8}.0\hat{ }{12,16}]heptadeca-1(10),11,13,16-tetraene (300 mg, 0.6 mmol, 1.0 eq), tetrahydrofuran (10 mL). This was followed by the addition of the above solution at −78° C. The resulting solution was stirred for 3 hours at 25° C. The reaction was then quenched by the addition of aqueous NH₄Cl (300 mL) and extracted with EtOAc (3×200 mL). The combined organic layer was washed with brine (300 mL), dried over anhydrous Na₂SO₄. After filtration, the filtrate was concentrated under vacuum. The crude residue was applied onto a silica gel column and eluted with ethyl acetate/petroleum ether=1:1 to give (3R,8S)-15-{[2-(trimethylsilyl)ethoxy]methyl}-2,5-dioxa-9,15,17-triazatetracyclo[8.7.0.0\hat{ }{3,8}.0\hat{ }{12,16}]heptadeca-1(10),11,13,16-tetraene as a yellow oil (120 mg, 57.06%). LC-MS (ESI, m/z) M+1: 362. ¹NHMR (300 MHz, Chloroform-d) δ 7.50 (s, 1H), 7.20 (d, J=3.5 Hz, 1H), 6.36 (d, J=3.6 Hz, 1H), 5.57 (s, 2H), 4.37 (dd, J=10.9, 5.0 Hz, 1H), 4.24-3.98 (m, 2H), 3.66-3.26 (m, 5H), 2.39-2.11 (m, 1H), 2.10-1.84 (m, 1H), 0.96-0.86 (m, 2H), −0.04 (s, 9H).

### Example INT_32 Preparation of N-[3-nitro-4-({[(1r,4r)-4-hydroxy-4-methylcyclohexyl]methyl}amino)benzenesulfonyl]-4-{2-oxo-7-azaspiro[3.5]nonan-7-yl}-2-[(3R,8S)-15-{[2-(trimethylsilyl)ethoxy]methyl}-2,5-dioxa-9,15,17-triazatetracyclo[8.7.0.0\hat{ }{3,8}.0\hat{ }{12,16}]heptadeca-1(10),11,13,16-tetraen-9-yl]benzamide

Synthesis of 2-bromo-N-[3-nitro-4-({[(1r,4r)-4-hydroxy-4-methylcyclohexyl]methyl}amino)benzenesulfonyl]-4-{2-oxo-7-azaspiro[3.5]nonan-7-yl}benzamide: Into a 40 mL vial, were placed 2-bromo-4-{2-oxo-7-azaspiro[3.5]nonan-7-yl}benzoic acid (470 mg, 1.4 mmol, 1.0 eq), 3-nitro-4-({[(1r,4r)-4-hydroxy-4-methylcyclohexyl]methyl}amino)benzenesulfonamide (477 mg, 1.4 mmol, 1.0 eq), dichloromethane (10 mL), EDCl (533 mg, 2.8 mmol, 2.0 eq), N,N-dimethylpyridin-4-amine (679 mg, 5.6 mmol, 4.0 eq). The reaction mixture was stirred overnight at 30° C. The resulting mixture was concentrated under vacuum. The crude residue was applied onto a silica gel column and eluted with dichloromethane/ethyl acetate (included 20% of methanol)=1:1 to give 2-bromo-N-[3-nitro-4-({[(1r,4r)-4-hydroxy-4-methylcyclohexyl]methyl}amino)benzenesulfonyl]-4-{2-oxo-7-azaspiro[3.5]nonan-7-yl}benzamide as a yellow solid (510 mg, 55.3%). ¹H NMR (300 MHz, DMSO-d₆) δ 12.27 (s, 1H), 8.70-8.53 (m, 2H), 7.92 (dd, J=9.3, 2.3 Hz, 1H), 7.36 (d, J=8.7 Hz, 1H), 7.27 (d, J=9.4 Hz, 1H), 7.11 (d, J=2.3 Hz, 1H), 6.95 (dd, J=8.9, 2.4 Hz, 1H), 4.24 (s, 1H), 3.33 (s, 7H), 2.84 (s, 4H), 1.81-1.61 (m, 7H), 1.55 (d, J=12.5 Hz, 1H), 1.43-0.98 (m, 7H).

Synthesis of N-[3-nitro-4-({[(1r,4r)-4-hydroxy-4-methylcyclohexyl]methyl}amino)benzenesulfonyl]-4-{2-oxo-7-azaspiro[3.5]nonan-7-yl}-2-[(3R,8S)-15-{[2-(trimethylsilyl)ethoxy]methyl}-2,5-dioxa-9,15,17-triazatetracyclo[8.7.0.0 \hat{ }{3,8}.0\hat{ }{12,16}]heptadeca-1(10),11,13,16-tetraen-9-yl]benzamide: Into a 40 mL vial, were placed (3R,8S)-15-{[2-(trimethylsilyl)ethoxy]methyl}-2,5-dioxa-9,15,17-triazatetracyclo[8.7.0.0\hat{ }{3,8}.0\hat{ }{12,16}]heptadeca-1(10),11,13,16-tetraene (210 mg, 0.6 mmol, 1.0 eq), 2-bromo-N-[3-nitro-4-({[(1r,4r)-4-hydroxy-4-methylcyclohexyl]methyl}amino)benzenesulfonyl]-4-{2-oxo-7-azaspiro[3.5]nonan-7-yl}benzamide (500 mg, 0.8 mmol, 1.3 eq), Dimethyl sulfoxide (10 mL), K₂CO₃ (241 mg, 1.7 mmol, 3.0 eq), Cul (55 mg, 0.3 mmol, 0.5 eq), N,N′-diphenyl-ethanediamide (70 mg, 0.3 mmol, 0.5 eq). The reaction mixture was stirred for 3 hours at 100° C. under nitrogen atmosphere. The resulting mixture was quenched by the addition of water (200 mL) and extracted with EtOAc (3×50 mL). The combined organic layer was washed with brine (200 mL), dried over anhydrous Na₂SO₄. After filtration, the filtrate was concentrated under vacuum. The crude residue was applied onto a silica gel column and eluted with dichloromethane/methanol=10:1 to give N-[3-nitro-4-({[(1r,4r)-4-hydroxy-4-methylcyclohexyl]methyl}amino)benzenesulfonyl]-4-{2-oxo-7-azaspiro[3.5]nonan-7-yl}-2-[(3R,8S)-15-{[2-(trimethylsilyl)ethoxy]methyl}-2,5-dioxa-9,15,17-triazatetracyclo[8.7.0.0\hat{ }{3,8}.0\hat{ }{12,16}]heptadeca-1(10),11,13,16-tetraen-9-yl]benzamide as a yellow solid (220 mg, 40.1%). LC-MS (ESI, m/z) M+1: 944.

### Example INT_36 Preparation of (3R)-3-(2-isopropoxyphenyl)piperazine-1-carboxylate (assumed) and (3S)-3-(2-isopropoxyphenyl)piperazine-1-carboxylate (assumed)

Synthesis of 2-(2-isopropoxyphenyl)pyrazine: Into a 500 mL round-bottom flask were added 2-isopropoxyphenylboronic acid (20.1 g, 115.2 mmol, 1.2 eq), Pd(dppf)Cl₂·CH₂Cl₂ (3.9 g, 4.8 mmol, 0.05 eq), K₂CO₃ (39.8 g, 288.1 mmol, 3.0 eq) and Dioxane/H₂O (200 mL/20 mL) at 25° C. The resulting mixture was stirred for 2 hours at 80° C. under nitrogen atmosphere. The resulting mixture was filtered, the filter cake was washed with EtOAc (3×100 mL). The filtrate was concentrated under vacuum. The resulting mixture was diluted with water (50 mL), and extracted with EtOAc (3×50 mL). The combined organic layers was washed with brine (50 mL), dried over anhydrous Na₂SO₄. After filtration, the filtrate was concentrated under vacuum. The crude residue was applied onto a silica gel column and eluted with ethyl acetate/petroleum ether=1:2 to give 2-(2-isopropoxyphenyl)pyrazine as a yellow solid (15.0 g, 72.9%). ¹H NMR (400 MHz, Chloroform-d) δ 9.25 (d, J=1.6 Hz, 1H), 8.67 (dd, J=2.6, 1.5 Hz, 1H), 8.46 (d, J=2.5 Hz, 1H), 7.87 (dd, J=7.7, 1.8 Hz, 1H), 7.41 (ddd, J=8.3, 7.3, 1.8 Hz, 1H), 7.21-6.95 (m, 2H), 4.66 (p, J=6.1 Hz, 1H), 1.36 (d, J=6.0 Hz, 6H).

Synthesis of 2-(2-isopropoxyphenyl)piperazine hydrochloride: Into a 1000 mL pressure tank reactor were added 2-(2-isopropoxyphenyl)pyrazine (20.0 g, 93.3 mmol, 1.0 eq), HCl (6 mL, 197.4 mmol, 2.1 eq) and EtOH (500 mL) at 25° C. The resulting mixture was stirred for overnight at 25° C. under hydrogen atmosphere (5 atm). The resulting mixture was filtered, the filter cake was washed with EtOH (3×30 mL). The filtrate was concentrated under vacuum. The crude resulting mixture was used in the next step directly without further purification (22.0 g crude). LC-MS (ESI, m/z) M+1: 221.

Synthesis of tert-butyl 3-(2-isopropoxyphenyl)piperazine-1-carboxylate: A solution of 2-(2-isopropoxyphenyl)piperazine hydrochloride (1.2 g, 5.447 mmol, 1.0 eq) in DCM (10 mL) was treated with TEA (1.6 g, 16.3 mmol, 3.0 eq). This was followed by the addition of Boc₂O (1.2 g, 5.4 mmol, 1.0 eq) dropwise at 0° C. The resulting mixture was stirred for 1 hour at 0° C. The reaction was quenched by the addition of water (5 mL) at 0° C. The resulting mixture was extracted with CH₂Cl₂ (3×3 mL). The combined organic layers were washed with brine (10 mL), dried over anhydrous Na₂SO₄. After filtration, the filtrate was concentrated under vacuum. The crude residue was applied onto a silica gel column and eluted with THE/petroleum ether=1:20 to give tert-butyl 3-(2-isopropoxyphenyl)piperazine-1-carboxylate as a brown oil (0.9 g, 48.7%). LC-MS (ESI, m/z) M+1: 321.

Synthesis of (3R)-3-(2-isopropoxyphenyl)piperazine-1-carboxylate (assumed) and (3S)-3-(2-isopropoxyphenyl)piperazine-1-carboxylate (assumed): The crude product (7.0 g, 21.8 mmol, 1.0 eq,) was purified by Prep-CHIRAL-SFC using the following conditions to give tert-butyl (3R)-3-(2-isopropoxyphenyl)piperazine-1-carboxylate (assumed) (2.4 g, 34.3%) and tert-butyl (3S)-3-(2-isopropoxyphenyl)piperazine-1-carboxylate (assumed) (3.1 g, 44.3%) as an off-white oil. A: RT1=3.4 min in CHIRAL ART Cellulose-SZ, 3*25 cm, 5 μm; Mobile Phase A: CO₂, Mobile Phase B: MeOH (0.1% 2M NH₃-MeOH); Flow rate: 80 mL/min; Gradient: isocratic 15% B; Column Temperature: 35° C.; Back Pressure: 100 bar; Wave Length: 220 nm; Sample Solvent: MeOH—HPLC; Injection Volume: 0.8 mL; Number of Runs: 80. B: RT1=4.1 min in CHIRAL ART Cellulose-SZ, 3*25 cm, 5 μm; Mobile Phase A: CO₂, Mobile Phase B: MeOH (0.1% 2M NH₃-MeOH); Flow rate: 80 mL/min; Gradient: isocratic 15% B; Column Temperature: 35° C.; Back Pressure: 100 bar; Wave Length: 220 nm; Sample Solvent: MeOH—H PLC; Injection Volume: 0.8 mL; Number of Runs: 80. LC-MS (ESI, m/z) M+1: 321. A-¹H NMR (300 MHz, DMSO-d₆) δ 7.61-7.37 (m, 1H), 7.21 (ddd, J=8.2, 7.3, 1.8 Hz, 1H), 7.04-6.95 (m, 1H), 6.90 (td, J=7.5, 1.1 Hz, 1H), 4.65 (p, J=6.0 Hz, 1H), 4.10 (q, J=5.2 Hz, 1H), 3.87 (dd, J=10.2, 3.1 Hz, 2H), 2.98 (d, J=10.5 Hz, 1H), 2.72-2.63 (m, 1H), 1.41 (s, 9H), 1.30 (d, J=6.0 Hz, 6H), 1.11-1.01 (m, 1H), 0.95-0.68 (m, 1H). B-¹H NMR (300 MHz, DMSO-d₆) δ 7.55-7.37 (m, 1H), 7.20 (ddd, J=8.8, 7.4, 1.8 Hz, 1H), 7.04-6.95 (m, 1H), 6.90 (td, J=7.5, 1.1 Hz, 1H), 4.64 (q, J=6.0 Hz, 1H), 4.07 (s, 2H), 3.87 (dd, J=10.2, 3.1 Hz, 2H), 3.02-2.87 (m, 1H), 2.80-2.58 (m, 1H), 2.38 (t, J=11.2 Hz, 1H), 1.41 (s, 9H), 1.30 (d, J=6.0 Hz, 6H).

### Example INT_37 (Method A) Preparation of (2R)-2-(2-methylphenyl)piperazine

Synthesis of 2-(o-tolyl)pyrazine: Into a 1 L 3-necked round-bottom flask, were added 2-methylphenylboronic acid (49.6 g, 364.9 mmol, 1.1 eq), dioxane (500 mL), water (50 mL), 2-chloropyrazine (38.0 g, 331.8 mmol, 1.0 eq), K₂CO₃ (137.6 g, 995.4 mmol, 3.0 eq), Pd(dppf)Cl₂·CH₂Cl₂ (27.0 g, 33.2 mmol, 0.0.1 eq). The resulting mixture was stirred for 2 hours at 80° C. under nitrogen atmosphere. The resulting mixture was filtered, the filter cake was diluted with water (200 mL), and extracted with ethyl acetate (3×500 mL). The combined organic layers were dried over anhydrous Na₂SO₄. After filtration, the filtrate was concentrated under vacuum. The crude residue was applied onto a silica gel column and eluted with petroleum ether/ethyl acetate=10:1 to give 2-(o-tolyl)pyrazine as a light yellow oil (40.0 g, 70.8%). LC-MS (ESI, m/z) M+1: 171.

Synthesis of 2-(o-tolyl)piperazine hydrochloride: To a solution of 2-(o-tolyl)pyrazine (40.0 g, 235.0 mmol, 1.0 eq) in THE (350 mL) and acetic acid (350 mL), was added Pd(OH)₂/C (10%, 4.0g) under nitrogen atmosphere in a 2 L pressure tank reactor. The mixture was hydrogenated at 25° C. under 10 atm of hydrogen pressure for overnight. The reaction mixture was filtered through a Celite pad. To the above mixture was added HCl/dioxane (300 mL), and stirred for 4 hours at 25° C. The precipitated solids were collected by filtration and washed with ethyl acetate (3×100 mL). The solid was concentrated under vacuum. Finally, 2-(o-tolyl)piperazine hydrochloride was obtained as a grey solid (40.0 g, 80.0%). LC-MS (ESI, m/z) M+1-HCl: 177.

Synthesis of tert-butyl 3-(2-methylphenyl)piperazine-1-carboxylate: Into a 250 mL 3-necked round-bottom flask, were added 2-(o-tolyl)piperazine hydrochloride (15.0 g, 70.7 mmol, 1.0 eq), DCM (200 mL). To the above mixture was added (Boc)₂O (10.7 g, 49.5 mmol, 0.7 eq) dropwise at 0° C. The resulting mixture was stirred for 1 h at 0° C. The reaction was quenched with water (100 mL) and extracted with dichloromethane (3×100 mL). The combined organic layers were dried over anhydrous Na₂SO₄. After filtration, the filtrate was concentrated under vacuum. The crude residue was applied onto a silica gel column and eluted with petroleum ether/ethyl acetate=1:3 to give tert-butyl 3-(2-methylphenyl)piperazine-1-carboxylate as a light yellow oil (10.0 g, 42.5%). LC-MS (ESI, m/z) M+1: 277.

Synthesis of tert-butyl (3R)-3-(2-methylphenyl)piperazine-1-carboxylate;tert-butyl (3S)-3-(2-methylphenyl)piperazine-1-carboxylate: tert-butyl 3-(2-methylphenyl)piperazine-1-carboxylate (800 mg) was purified by Chiral-SFC using the following conditions: CHIRALPAK IG, 3*25 cm, 5 um; Mobile Phase A: CO₂, Mobile Phase B: MeOH (0.1% 2M NH₃-MeOH); Flow rate: 80 mL/min; Gradient: isocratic 20% B; Wave Length: 220 nm; RT1(min): 3.1; RT₂(min): 3.9; Sample Solvent: MeOH—HPLC; Injection Volume: 4 mL; Number Of Runs: 10. Finally, tert-butyl (3R)-3-(2-methylphenyl)piperazine-1-carboxylate (385 mg, 48.1%) was obtained as a light yellow oil (385 mg, 48.1%) and tert-butyl (3S)-3-(2-methylphenyl)piperazine-1-carboxylate was obtained as a light yellow oil (380 mg, 47.5%). A: RT1=0.396 min in CHIRALPACK IG-3 50*3.0 mm, 3.0 um; Mobile Phase A: CO₂, Mobile Phase B: MeOH (20 m MNH₃); Gradient: 10% to 50%_B1_2_2200; Column Temperature: 35° C.; Back Pressure: 2200 bar; Wave Length: 220 nm; Injection Volume: 1 uL. B: RT2=0.524 min in CHIRALPACK IG-3 50*3.0 mm, 3.0 um; Mobile Phase A: CO₂, Mobile Phase B: MeOH (20 m MNH₃); Gradient: 10% to 50%_B1_2_2200; Column Temperature: 35° C.; Back Pressure: 2200bar; Wave Length: 220 nm; Injection Volume: 1 uL. LC-MS (ESI, m/z) M+1: 321.

Synthesis of (2R)-2-(2-methylphenyl)piperazine: Into an 8 mL vial, were added tert-butyl (3R)-3-(2-methylphenyl)piperazine-1-carboxylate (380 mg, 1.4 mmol, 1.0 eq), HCl/1,4-dioxane (3 mL). The resulting mixture was stirred for 3 hours at 25° C. The mixture was neutralized to pH=7 with saturated NaHCO₃. The resulting mixture was extracted with dichloromethane (3×10 mL). The combined organic layers were dried over anhydrous Na₂SO₄. After filtration, the filtrate was concentrated under vacuum. Finally, (2R)-2-(2-methylphenyl)piperazine was obtained as a light yellow oil (200 mg, 82.5%). LC-MS (ESI, m/z) M+1: 177.

### Example INT_37 (Method B) Preparation of (2R)-2-(2-methylphenyl)piperazine

Synthesis of (2R)-2-(2-methylphenyl)piperazine: Into a 2 L 4-necked round-bottom flask were added 2-(o-tolyl)piperazine hydrochloride (90.0 g, 510.6 mmol, 1.0 eq), ethyl acetate (1350 mL), methanol (135 mL) The resulting mixture was stirred for 20 min at 50° C. To the above mixture was added (S)-mandelic acid (77.7 g, 510.6 mmol, 1.0 eq). The resulting mixture was stirred for 30 min at 70° C. To the above mixture was added methanol (90 mL) at 70° C. The resulting mixture was stirred for additional 2 hours at 70° C. The mixture was allowed to cool down to 25° C. and stirred for overnight at 25° C. The precipitated solids were collected by filtration. The residue was neutralized to pH 8 with saturated Na₂CO₃ (aq.). The resulting mixture was extracted with dichloromethane (3×500 mL) and dried over anhydrous Na₂SO₄. After filtration, the filtrate was concentrated under vacuum. Finally, (2R)-2-(2-methylphenyl)piperazine was obtained as a colorless oil (30.0 g, 33.3%). A: RT1=0.569 min in CHIRALPACK IH-3 50*3.0 mm, 3.0 um; Mobile Phase A: CO₂, Mobile Phase B: MeOH (20 m MNH₃); Gradient: 10% to 50%_B1_2_2200; Column Temperature: 35° C.; Back Pressure: 2200 bar; Wave Length: 220 nm; Injection Volume: 0.5 uL. LC-MS (ESI, m/z) M+1: 177.

### Example INT_38 Preparation of 5-bromo-3,6-difluoro-1-((2-(trimethylsilyl)ethoxy)methyl)-1H-pyrrolo[2,3-b]pyridine

Synthesis of 5-bromo-6-fluoropyridin-2-amine: Into a 10 L 3-necked round-bottom flask were placed 6-fluoropyridin-2-amine (500.0 g, 4460.0 mmol, 1.0 eq) and MeCN (5 L) at 25° C. To the above mixture was added NBS (817.6 g, 4593.8 mmol, 1.0 eq) in portions over 30 min at 15° C. The resulting mixture was stirred for 2 hours at 25° C. The reaction mixture was quenched by the addition of water (500 mL). The resulting mixture was concentrated under vacuum to remove MeCN. The resulting mixture was extracted with EtOAc (2×5 L). The combined organic layers were washed with brine (5 L), dried over anhydrous Na₂SO₄. After filtration, the filtrate was concentrated under vacuum. The residue was purified by trituration with petroleum ether (2 L). The crude residue was applied onto a silica gel column and eluted with ethyl acetate/petroleum ether=1:1 to give 5-bromo-6-fluoropyridin-2-amine as an off-white solid (800.0 g, 93.9%). LC-MS (ESI, m/z) M+1: 191/193. ¹H NMR (300 MHz, DMSO-d₆) δ 7.64 (dd, J=9.6, 8.4 Hz, 1H), 6.56 (bs, 2H), 6.29 (dd, J=8.5, 1.7 Hz, 1H).

Synthesis of ethyl 2-(2-amino-5-bromo-6-fluoropyridin-3-yl)-2,2-difluoroacetate: Into a 5 L 3-necked round-bottom flask were added 5-bromo-6-fluoropyridin-2-amine (200.0 g, 1047.1 mmol, 1.0 eq), DMSO (2 L), ethyl 2-bromo-2,2-difluoroacetate (637.6 g, 3141.3 mmol, 3.0 eq) and ferrocene (19.5 g, 104.7 mmol, 0.1 eq) at 25° C. To the above mixture was added H₂O₂ (30%) (237.4 g, 2094.2 mmol, 2.0 eq) dropwise over 1 hour at 20° C. The resulting mixture was stirred for additional 4 hours at 25° C. The reaction mixture was quenched by the addition of water/ice (2 L). The precipitated solids were collected by filtration. The residue was dissolved in EtOAc (1 L) washed with aqueous Na₂SO₃ (1 L), brine (1 L). The combined organic layers were dried over anhydrous Na₂SO₄. After filtration, the filtrate was concentrated under vacuum. The crude residue was applied onto a silica gel column and eluted with ethyl acetate/petroleum ether=1:1 to give ethyl 2-(2-amino-5-bromo-6-fluoropyridin-3-yl)-2,2-difluoroacetate as an off-white solid (150.0 g, 45.8%). LC-MS (ESI, m/z) M+1: 313/315. ¹H NMR (300 MHz, DMSO-d₆) δ 7.92 (d, J=8.5 Hz, 1H), 6.75 (bs, 2H), 4.35 (q, J=7.1 Hz, 2H), 1.26 (t, J=7.1 Hz, 3H).

Synthesis of 5-bromo-3,3,6-trifluoro-1,3-dihydro-2H-pyrrolo[2,3-b]pyridin-2-one: Into a 10 L 3-necked round-bottom flask were placed ethyl 2-(2-amino-5-bromo-6-fluoropyridin-3-yl)-2,2-difluoroacetate (150.0 g, 479.1 mmol, 1.0 eq), DMSO (4500 mL) and Cs₂CO₃ (312.2 g, 958.2 mmol, 2.0 eq) at 25° C. The resulting mixture was stirred for 2 hours at 50° C. The reaction was quenched by the addition of HCl (0.5 M, 3 L) and extracted with EtOAc (2×3 L). The combined organic layers were washed with brine (5 L), dried over anhydrous Na₂SO₄. After filtration, the filtrate was concentrated under vacuum. The residue was purified by trituration with petroleum ether (1 L). The crude residue was applied onto a silica gel column and eluted with ethyl acetate/petroleum ether=1:1 to give 5-bromo-3,3,6-trifluoro-1,3-dihydro-2H-pyrrolo[2,3-b]pyridin-2-one as a yellow solid (110.0 g, 86.0%). LC-MS (ESI, m/z) M-1: 265/267. ¹H NMR (300 MHz, DMSO-d₆) δ 12.25 (bs, 1H), 8.71 (dt, J=8.3, 1.5 Hz, 1H).

Synthesis of 5-bromo-3,6-difluoro-1H-pyrrolo[2,3-b]pyridine: Into a 2 L 3-necked round-bottom flask were placed 5-bromo-3,3,6-trifluoro-1,3-dihydro-2H-pyrrolo[2,3-b]pyridin-2-one (50.0 g, 187.3 mmol, 1.0 eq). Then BH₃-THF (1 L, 1.0 mol, 5.3 eq) was added dropwise over 1 hour at 0° C. The resulting mixture was stirred for 3 hours at 25° C. The reaction was quenched by the addition of NaOH (aq.) (500 mL, 1 M) at 0° C. The resulting mixture was extracted with CH₂Cl₂ (2×500 mL). The combined organic layers were washed with brine (1 L), dried over anhydrous Na₂SO₄. After filtration, the filtrate was concentrated under vacuum. The crude residue was applied onto a silica gel column and eluted with petroleum ether/ethyl acetate=10:1 to give 5-bromo-3,6-difluoro-1H-pyrrolo[2,3-b]pyridine as a colorless oil (11.0 g, 25.2%). LC-MS (ESI, m/z) M-1: 231/233.

Synthesis of 5-bromo-3,6-difluoro-1-((2-(trimethylsilyl)ethoxy)methyl)-1H-pyrrolo[2,3-b]pyridine: Into a 500 mL 3-necked round-bottom flask were added 5-bromo-3,6-difluoro-1H-pyrrolo[2,3-b]pyridine (10.0 g, 42.9 mmol, 1.0 eq) and THE (200 mL) at 25° C. To the above mixture was added NaH (3.4 g, 85.8 mmol, 2.0 eq, 60%) in portions at 0° C. The resulting mixture was stirred for 30 min at 0° C. To the above mixture was added SEM-Cl (10.7 g, 64.4 mmol, 1.5 eq) dropwise at 0° C. The resulting mixture was stirred for additional 2 hours at 25° C. The reaction was quenched by the addition of sat. NH₄Cl (50 mL) at 0° C. The resulting mixture was extracted with EtOAc (2×200 mL). The combined organic layers were washed with brine (300 mL), dried over anhydrous Na₂SO₄. After filtration, the filtrate was concentrated under vacuum. The crude residue was applied onto a silica gel column and eluted with petroleum ether/ethyl acetate=12:1 to give 5-bromo-3,6-difluoro-1-((2-(trimethylsilyl)ethoxy)methyl)-1H-pyrrolo[2,3-b]pyridine as a colorless oil (10.0 g, 64.1%). LC-MS (ESI, m/z) M+1: 363/365. ¹H NMR (300 MHz, Chloroform-d) δ 8.20 (dd, J=8.4, 0.6 Hz, 1H), 7.09 (d, J=2.5 Hz, 1H), 5.53 (s, 2H), 3.59-3.40 (m, 2H), 1.00-0.79 (m, 2H), −0.03 (s, 9H).

### Example INT_39 Preparation of (S)-5-methoxy-6-(3-methylmorpholino)nicotinaldehyde

Synthesis of (S)-4-(3-methoxypyridin-2-yl)-3-methylmorpholine: Into a 100 mL round-bottom flask were added 2-bromo-3-methoxypyridine (5.0 g, 26.6 mmol, 1.0 eq), (3S)-3-methylmorpholine (5.4 g, 53.2 mmol, 2.0 eq), DMSO (30 mL) and Cs₂CO₃ (13.0 g, 40.0 mmol, 1.5 eq). The resulting mixture was stirred for 24 hours at 140° C. under nitrogen atmosphere. The resulting mixture was then quenched by the addition of water (20 mL) and then extracted with EtOAc (3×50 mL). The combined organic layers were washed with brine (100 mL), dried over anhydrous Na₂SO₄. After filtration, the filtrate was concentrated under vacuum. The crude residue was applied onto a silica gel column and eluted with ethyl acetate/petroleum ether=1:1 to give (S)-4-(3-methoxypyridin-2-yl)-3-methylmorpholine as a colorless oil (1.0 g, 18.1%). ¹H NMR (300 MHz, Chloroform-d) δ 7.93 (dd, J=5.0, 1.5 Hz, 1H), 7.10 (d, J=7.9 Hz, 1H), 6.89 (dd, J=7.9, 5.0 Hz, 1H), 4.22 (s, 1H), 4.03-3.83 (m, 3H), 3.88 (s, 3H), 3.83-3.35 (m, 3H), 1.17 (d, J=6.6 Hz, 3H).

Synthesis of (S)-4-(5-bromo-3-methoxypyridin-2-yl)-3-methylmorpholine: Into a 50 mL round-bottom flask were added (S)-4-(3-methoxypyridin-2-yl)-3-methylmorpholine (900 mg, 4.3 mmol, 1.0 eq) and DMF (20 mL) at 25° C. To the above mixture was added NBS (769 mg, 4.3 mmol, 1.0 eq) in portions at 25° C. The resulting mixture was stirred for 4 hours at 80° C. The reaction was quenched by the addition of water (5 mL) at 25° C. The resulting mixture was extracted with EtOAc (2×30 mL). The combined organic layers were washed with brine (50 mL), dried over anhydrous Na₂SO₄. After filtration, the filtrate was concentrated under vacuum. The crude residue was applied onto a silica gel column and eluted with ethyl acetate/petroleum ether=1:1 to give (S)-4-(5-bromo-3-methoxypyridin-2-yl)-3-methylmorpholine as a white solid (600 mg, 48.4%). LC-MS (ESI, m/z) M+1: 287/289.

Synthesis of (S)-5-methoxy-6-(3-methylmorpholino)nicotinaldehyde: Into a 50 mL 3-necked round-bottom flask were added (S)-4-(5-bromo-3-methoxypyridin-2-yl)-3-methylmorpholine (600 mg, 2.1 mmol, 1.0 eq) and THE (10 mL) at 25° C. To the above mixture was added n-BuLi in hexanes (1 mL, 2.5 mmol, 1.2 eq) dropwise at −78° C. The resulting mixture was stirred for 1 hour at −78° C. To the above mixture was added DMF (916 mg, 12.5 mmol, 6.0 eq) dropwise at −78° C. The resulting mixture was stirred for additional 1 hour at −78˜25° C. The reaction was quenched by the addition of sat. NH₄Cl (1 mL) and then extracted with EtOAc (2×20 mL). The combined organic layers were washed with brine (30 mL), dried over anhydrous Na₂SO₄. After filtration, the filtrate was concentrated under vacuum. The crude residue was applied onto a silica gel column and eluted with ethyl acetate/petroleum ether=1:1 to give (S)-5-methoxy-6-(3-methylmorpholino)nicotinaldehyde as a colorless oil (310 mg, 62.8%). LC-MS (ESI, m/z) M+1: 237.

### Example INT_40 Preparation of 3,3-dimethyl-2,3-dihydro-[1,4]dioxino[2,3-b]pyridine-8-carbaldehyde

Synthesis of ethyl 2-[(2-chloropyridin-3-yl)oxy]acetate: Into a 500-mL round-bottom flask, was placed 2-chloropyridin-3-ol (20 g, 154.4 mmol, 1.0 eq), Cs₂CO₃ (125.8 g, 386.0 mmol, 2.5 eq), MeCN (200 mL), ethyl bromoacetate (25.8 g, 154.4 mmol, 1.0 eq). The reaction mixture was stirred for 2 hours at 30° C. The resulting mixture was concentrated under vacuum. The crude residue was applied onto a silica gel column and eluted with ethyl acetate/petroleum ether=1:3 to give ethyl 2-[(2-chloropyridin-3-yl)oxy]acetate as a white solid (30.0 g, 90.1%). ¹H NMR (300 MHz, Chloroform-d) δ 8.08 (dd, J=4.4, 1.8 Hz, 1H), 7.27-7.13 (m, 2H), 4.75 (s, 2H), 4.29 (q, J=7.2 Hz, 2H), 1.31 (t, J=7.1 Hz, 3H).

Synthesis of 1-[(2-chloropyridin-3-yl)oxy]-2-methylpropan-2-ol: Into a 500 mL 3-necked round-bottom flask purged and maintained with an inert atmosphere of nitrogen, were placed ethyl 2-[(2-chloropyridin-3-yl)oxy]acetate (20.0 g, 92.8 mmol, 1.0 eq), THE (200 mL). This was followed by the addition of MeMgBr (55.3 g, 463.8 mmol, 5.0 eq) in portions at 0° C. The reaction mixture was stirred for 1 hours at 0° C. The reaction was then quenched by the addition of aqueous NH₄Cl (30 mL). The resulting mixture was concentrated in vacuo. The residue was dissolved in dichloromethane (500 mL) and washed with water (3×50 mL). The organic layer was dried over Na₂SO₄. After filtration, the filtrate was concentrated under vacuum to give 1-[(2-chloropyridin-3-yl)oxy]-2-methylpropan-2-ol as a colorless oil (16.0 g, 85.5%). ¹H NMR (300 MHz, Chloroform-d) δ 8.02 (dd, J=3.5, 2.8 Hz, 1H), 7.24-7.13 (m, 2H), 3.88 (s, 2H), 1.41 (s, 6H).

Synthesis of 3,3-dimethyl-2,3-dihydro-[1,4]dioxino[2,3-b]pyridine: Into a 1000 mL round-bottom flask were added 1-[(2-chloropyridin-3-yl)oxy]-2-methylpropan-2-ol (16.0 g, 79.3 mmol, 1.0 eq) and dioxane (300 mL) at 25° C. To the above mixture was added NaH (12.7 g, 317.4 mmol, 4.0 eq, 60%) in portions at 0° C. The resulting mixture was stirred for additional 16 hours at 90° C. The reaction was quenched by the addition of water/ice (50 mL) at 0° C. The resulting mixture was extracted with EtOAc (2×500 mL). The combined organic layers were washed with brine (1 L), dried over anhydrous Na₂SO₄. After filtration, the filtrate was concentrated under vacuum. The crude residue was applied onto a silica gel column and eluted with ethyl acetate/petroleum ether=1:1 to give 3,3-dimethyl-2,3-dihydro-[1,4]dioxino[2,3-b]pyridine as a colorless oil (3.0 g, 22.9%). ¹H NMR (300 MHz, Chloroform-d) δ 7.82 (dd, J=4.8, 1.7 Hz, 1H), 7.18 (dd, J=7.8, 1.6 Hz, 1H), 6.83 (dd, J=7.9, 4.8 Hz, 1H), 3.88 (s, 2H), 1.41 (s, 6H).

Synthesis of 3,3-dimethyl-2,3-dihydro-[1,4]dioxino[2,3-b]pyridine-8-carbaldehyde: Into a 250 mL 3-necked round-bottom flask were added 3,3-dimethyl-2,3-dihydro-[1,4]dioxino[2,3-b]pyridine (2.0 g, 12.1 mmol, 1.0 eq) and THE (60 mL) at 25° C. To the above mixture was added n-BuLi in hexanes (12 mL, 30.3 mmol, 2.5 eq) dropwise at −78° C. The resulting mixture was slowly warmed up to 0° C. and stirred for additional 1 hour at 0° C. To the above mixture was added DMF (4.0 g, 54.5 mmol, 4.5 eq) dropwise at −78° C. The resulting mixture was slowly warmed up to 0° C. and stirred for additional 30 min at 0° C. The reaction was quenched by the addition of sat. NH₄Cl (10 mL) at 0° C. The resulting mixture was extracted with EtOAc (2×50 mL). The combined organic layers were washed with brine (50 mL), dried over anhydrous Na₂SO₄. After filtration, the filtrate was concentrated under vacuum. The crude residue was applied onto a silica gel column and eluted with ethyl acetate/petroleum ether=1:1 to give 3,3-dimethyl-2H-[1,4]dioxino[2,3-b]pyridine-8-carbaldehyde as a white solid (420 mg, 18.0%). ¹H NMR (300 MHz, DMSO-d₆) δ 10.37 (d, J=0.7 Hz, 1H), 7.89 (dd, J=5.0, 0.7 Hz, 1H), 7.18 (d, J=5.0 Hz, 1H), 4.16 (s, 2H), 1.39 (s, 6H).

### Example INT_41 Preparation of 5-morpholinopicolinaldehyde

Synthesis of 5-fluoropicolinaldehyde: Into a 250 mL round-bottom flask, a solution of 2-bromo-5-fluoropyridine (10.0 g, 36.3 mmol, 1.0 eq) in THE (100 mL) was treated with n-BuLi (2.3 g, 40.1 mmol, 1.1 eq) for 1 hour at −78° C. under nitrogen atmosphere followed by the addition of DMF (3.6 g, 21.9 mmol, 1.2 eq) in portions at −78° C. The resulting mixture was stirred for 2 hours at −78° C. under N₂ atmosphere. The reaction was quenched by the addition of NH₄Cl. aq (50 mL) at 0° C. The resulting mixture was extracted with ethyl acetate (3×100 mL). The combined organic layers were washed with water (3×30 mL), and then dried over anhydrous Na₂SO₄. After filtration, the filtrate was concentrated under vacuum. The crude residue was purified onto a silica gel column and eluted with ethyl acetate/petroleum ether=1:1 to give 5-fluoropicolinaldehyde as a yellow solid (5.0 g, 49.2%).

Synthesis of 5-morpholinopicolinaldehyde: Into a 250 mL round-bottom flask were added 5-fluoropicolinaldehyde (5.0 g, 40.8 mmol, 1.0 eq), morpholine (8.6 g, 80.8 mmol, 2.0 eq) and K₂CO₃ (16 g, 120.8 mmol, 3.0 eq) in CH₃CN (50 mL) at 25° C. The resulting mixture was stirred for 16 hours at 80° C. The resulting mixture was washed with water (3×50 mL). The aqueous layer was extracted with ethyl acetate (3×50 mL). The combined organic layers was dried over anhydrous Na₂SO₄. After filtration, the filtrate was concentrated under vacuum. The crude residue was purified onto a silica gel column and eluted with ethyl acetate/petroleum ether=1:1 to give 5-morpholinopicolinaldehyde as a yellow oil (2.0 g, 44.5%). LC-MS (ESI, m/z) M+1: 193.

### Example INT_42 Preparation of 7-methoxy-2-methylbenzofuran-5-carbaldehyde

Synthesis of 4-(allyloxy)-3-methoxybenzaldehyde: Into a 250 mL round-bottom flask, To a stirred solution of 4-hydroxy-3-methoxybenzaldehyde (20.3 g, 144.8 mmol, 1.0 eq) and allyl bromide (35.3 g, 289.0 mmol, 2.0 eq) in acetone (200 mL) was added K₂CO₃ (60.4 g, 434.0 mmol, 3.0 eq) in portions at 25° C. The resulting mixture was stirred for 16 hours at 25° C. The reaction was quenched by the addition of water (200 mL) at 25° C. The resulting mixture was extracted with ethyl acetate (3×200 mL). The combined organic layers were washed with brine (2×100 mL), dried over anhydrous Na₂SO₄. After filtration, the filtrate was concentrated under reduced pressure. The crude residue was applied onto a silica gel column and eluted with ethyl acetate/petroleum ether=1:1 to give 4-(allyloxy)-3-methoxybenzaldehyde as a white solid (16.1 g, 57.9%). LC-MS (ESI, m/z) M+1: 193.

Synthesis of 3-allyl-4-hydroxy-5-methoxybenzaldehyde: Into a 500 mL round-bottom flask were added 4-(allyloxy)-3-methoxybenzaldehyde (15.3 g, 144.8 mmol, 1.0 eq) at 25° C. The final reaction mixture was irradiated with microwave radiation for 16 hours at 190° C. The crude residue was applied onto a silica gel column and eluted with ethyl acetate/petroleum ether=1:1 to give 3-allyl-4-hydroxy-5-methoxybenzaldehyde as a white solid (8.2 g, 53.3%). LC-MS (ESI, m/z) M+1: 193.

Synthesis of 2-(iodomethyl)-7-methoxy-2,3-dihydrobenzofuran-5-carbaldehyde: Into a 250 mL round-bottom flask, to a stirred solution of 3-allyl-4-hydroxy-5-methoxybenzaldehyde (8.2 g, 36.4 mmol, 1.0 eq) in THE (100 mL) was added I₂ (1.8 g, 7.4 mmol, 0.2 eq) and NaHCO₃ (18.8 g, 107.4 mmol, 3.0 eq) in portions at 25° C. The resulting mixture was stirred for 16 hours at 50° C. The reaction was quenched by the addition of water (100 mL) at 25° C. The resulting mixture was extracted with ethyl acetate (3×70 mL). The combined organic layers were washed with brine (2×50 mL), dried over anhydrous Na₂SO₄. After filtration, the filtrate was concentrated under reduced pressure. The crude residue was purified onto a silica gel column and eluted with ethyl acetate/petroleum ether=1:2 to give 2-(iodomethyl)-7-methoxy-2,3-dihydrobenzofuran-5-carbaldehyde as a white solid (5 g, 43.6%). LC-MS (ESI, m/z) M+1: 319.

Synthesis of 7-methoxy-2-methylbenzofuran-5-carbaldehyde: Into a 250 mL round-bottom flask were added 2-(iodomethyl)-7-methoxy-2,3-dihydrobenzofuran-5-carbaldehyde (300 mg, 0.9 mmol, 1.0 eq), Al₂O₃ (30.0 g) and CH₂Cl₂ (30 mL) at 25° C. The resulting mixture was concentrated under reduced pressure. The resulting mixture was stirred for 30 min at 150° C. The mixture was allowed to cool down to 25° C. The resulting mixture was washed with CH₂Cl₂ (3×100 mL). The precipitated solids were collected by filtration and washed with CH₂Cl₂ (3×100 mL). The resulting mixture was concentrated under reduced pressure. The crude residue was purified onto a silica gel column and eluted with ethyl acetate/petroleum ether=1:1to give 7-methoxy-2-methylbenzofuran-5-carbaldehyde as a yellow solid (50 mg, 27.8%). LC-MS (ESI, m/z) M+1: 191.

### Example INT_43 Preparation of 5-methoxy-6-morpholinonicotinaldehyde

Synthesis of 4-(3-methoxypyridin-2-yl)morpholine: Into a 250 mL round-bottom flask were added 2-bromo-3-methoxypyridine (15.0 g, 79.8 mmol, 1.0 eq) and morpholine (100 mL) at 25° C. The resulting mixture was stirred for 48 hours at 125° C. The resulting mixture was washed with water (3×100 mL). The aqueous layer was extracted with ethyl acetate (3×100 mL). The mixture was dried over anhydrous Na₂SO₄. After filtration, the filtrate was concentrated under vacuum. The crude residue was purified onto a silica gel column and eluted with ethyl acetate/petroleum ether=1:1 to give 4-(3-methoxypyridin-2-yl)morpholine as a yellow oil (10 g, 64.5%). LC-MS (ESI, m/z) M+1: 195.

Synthesis of 4-(5-bromo-3-methoxypyridin-2-yl)morpholine: Into a 250 mL round-bottom flask were added 4-(3-methoxypyridin-2-yl)morpholine (10.0 g, 51.5 mmol, 1.0 eq), NBS (11.9 g, 66.9 mmol, 1.3 eq) and DMF (100 mL). The resulting mixture was stirred for 20 hours at 80° C. The resulting mixture was extracted with ethyl acetate (3×100 mL). The combined organic layers were washed with water (3×100 mL), and dried over anhydrous Na₂SO₄. After filtration, the filtrate was concentrated under vacuum. The crude residue was purified onto a silica gel column and eluted with ethyl acetate/petroleum ether=1:1 to give 4-(5-bromo-3-methoxypyridin-2-yl)morpholine as a yellow oil (5.0 g, 35.6%).

Synthesis of 5-methoxy-6-morpholinonicotinaldehyde: Into a 250 mL round-bottom flask, a solution of 4-(5-bromo-3-methoxypyridin-2-yl)morpholine (5.0 g, 18.3 mmol, 1.0 eq) in THE (100 mL) was treated with n-BuLi (1.3 g, 20.1 mmol, 1.1 eq) for 1 hour at −78° C. under nitrogen atmosphere. This was followed by the addition of DMF (1.6 g, 21.9 mmol, 1.2 eq) in portions at −78° C. The resulting mixture was stirred for 2 hours at −78° C. under N₂ atmosphere. The reaction was quenched by the addition of sat. NH₄Cl (30 mL) at 0° C. The resulting mixture was extracted with ethyl acetate (3×50 mL). The combined organic layers were washed with water (3×15 mL), dried over anhydrous Na₂SO₄. After filtration, the filtrate was concentrated under vacuum. The crude residue was purified onto a silica gel column and eluted with ethyl acetate/petroleum ether=1:1 to give 5-methoxy-6-morpholinonicotinaldehyde as a yellow solid (2.0 g, 49.2%). LC-MS (ESI, m/z) M+1: 223.

### Example INT_44 Preparation of 6-cyclopropyl-5-methoxypyridine-3-carbaldehyde

Synthesis of 5-bromo-2-iodo-3-methoxypyridine: Into a 250 mL round-bottom flask, were added 5-bromo-2-iodopyridin-3-ol (10.0 g, 33.3 mmol, 1.0 eq), DMF (100 mL). K₂CO₃ (13.8 g, 100.0 mmol, 3.0 eq), CH₃I (5.7 g, 40.0 mmol, 1.2 eq). The resulting mixture was stirred for 2 hours at 25° C. The reaction was quenched with water (100 mL) and extracted with ethyl acetate (3×100 mL). The combined organic layers were dried over anhydrous Na₂SO₄. After filtration, the filtrate was concentrated under vacuum. The residue was purified by silica gel column chromatography and eluted with petroleum ether/ethyl acetate=1:1 to give 5-bromo-2-iodo-3-methoxypyridine as a colorless oil (9.2 g, 87.8%). LC-MS (ESI, m/z) M+1: 314/316.

Synthesis of 5-bromo-2-cyclopropyl-3-methoxypyridine: Into a 100 mL round-bottom flask, were added 5-bromo-2-iodo-3-methoxypyridine (4.2 g, 13.4 mmol, 1.0 eq), toluene (50 mL), water (5 mL), cyclopropylboronic acid (1.7 g, 20.0 mmol, 1.5 eq), Pd(OAc)₂ (300 mg, 1.3 mmol, 0.1 eq), PCy₃ (750 mg, 2.6 mmol, 0.2 eq). The mixture was stirred for 2 hours at 100° C. under nitrogen atmosphere. The resulting mixture was extracted with ethyl acetate (3×50 mL). The combined organic layers were dried over anhydrous Na₂SO₄. After filtration, the filtrate was concentrated under vacuum. The residue was purified by silica gel column chromatography and eluted with petroleum ether/ethyl acetate=1:1 to give 5-bromo-2-cyclopropyl-3-methoxypyridine as a white solid (1.0 g, 32.7%). LC-MS (ESI, m/z) M+1: 228/230.

Synthesis of 6-cyclopropyl-5-methoxypyridine-3-carbaldehyde: Into a 50 mL 3-necked round-bottom flask, were added 5-bromo-2-cyclopropyl-3-methoxypyridine (600 mg, 2.6 mmol, 1.0 eq), THE (10 mL). To the above mixture was added n-BuLi (1.2 mL, 3.2 mmol, 1.2 eq) dropwise at −78° C. under nitrogen atmosphere. The resulting mixture was stirred for 1 hour at −78° C. To the above mixture was added DMF (576 mg, 7.8 mmol, 3.0 eq) dropwise at −78° C. The reaction was quenched with sat. NH₄Cl at 0° C. The resulting mixture was extracted with ethyl acetate (3×10 mL). The combined organic layers were dried over anhydrous Na₂SO₄. After filtration, the filtrate was concentrated under vacuum. The residue was purified by silica gel column chromatography and eluted with petroleum ether/ethyl acetate=1:1 to give6-cyclopropyl-5-methoxypyridine-3-carbaldehyde as a colorless oil (260 mg, 55.7%). LC-MS (ESI, m/z) M+1: 178.

### Example INT_45 Preparation of 5-methoxy-6-methylpyridine-3-carbaldehyde

Synthesis of 5-bromo-2-iodo-3-methoxypyridine: Into a 250 mL round-bottom flask, were added 5-bromo-2-iodopyridin-3-ol (10.0 g, 33.3 mmol, 1.0 eq), DMF (100 mL). K₂CO₃ (13.8 g, 100.0 mmol, 3.0 eq), CH₃I (5.7 g, 40.0 mmol, 1.2 eq). The resulting mixture was stirred for 2 hours at 25° C. The reaction was quenched with water (100 mL) and extracted with ethyl acetate (3×100 mL). The combined organic layers were dried over anhydrous Na₂SO₄. After filtration, the filtrate was concentrated under vacuum. The residue was purified by silica gel column chromatography and eluted with petroleum ether/ethyl acetate=1:1 to give 5-bromo-2-iodo-3-methoxypyridine as a colorless oil (9.2 g, 87.8%). LC-MS (ESI, m/z) M+1: 314/316.

Synthesis of 5-bromo-3-methoxy-2-methylpyridine: Into a 40 mL vial, were added 5-bromo-2-iodo-3-methoxypyridine (300 mg, 1.0 mmol, 1.0 eq), toluene (5 mL), water (0.5 mL), potassium trimethylboranuide (459 mg, 5.0 mmol, 5.0 eq), K₃PO₄ (608 mg, 3.0 mmol, 3.0 eq), PCy₃ (27 mg, 0.1 mmol, 0.1 eq), Pd(OAc)₂ (43 mg, 0.2 mmol, 0.2 eq). The resulting mixture was stirred for 2 hours at 100° C. under nitrogen atmosphere. The resulting mixture was extracted with ethyl acetate (3×10 mL). The combined organic layers were dried over anhydrous Na₂SO₄. After filtration, the filtrate was concentrated under vacuum. The residue was purified by silica gel column chromatography and eluted with petroleum ether/ethyl acetate=1:1 to give 5-bromo-3-methoxy-2-methylpyridine as a colorless oil (62 mg, 32.1%). LC-MS (ESI, m/z) M+1: 202/204.

Synthesis of 5-methoxy-6-methylpyridine-3-carbaldehyde: Into a 50 mL 3-necked round-bottom flask, were added 5-bromo-3-methoxy-2-methylpyridine (300 mg, 1.5 mmol, 1.0 eq), THE (10 mL). To the above mixture was added n-BuLi (0.9 mL, 2.2 mmol, 1.5 eq) dropwise at −78° C. under nitrogen atmosphere. The resulting mixture was stirred for additional 1 hour at −78° C. This was followed by the addition of DMF (326 mg, 4.5 mmol, 3.0 eq) dropwise at −78° C. The reaction was quenched with sat. NH₄Cl at 0° C. The resulting mixture was extracted with ethyl acetate (3×10 mL). The combined organic layers were dried over anhydrous Na₂SO₄. After filtration, the filtrate was concentrated under vacuum. The residue was purified by Prep-TLC petroleum ether/ethyl acetate=1:1 to give 5-methoxy-6-methylpyridine-3-carbaldehyde as a colorless oil (65 mg, 29.0%). LC-MS (ESI, m/z) M+1: 152.

### Example INT_46 Preparation of 5-cyclopropyl-6-methoxypyridine-3-carbaldehyde

Synthesis of 3-cyclopropyl-2-methoxypyridine: Into a 500-mL round-bottom flask purged and maintained with an inert atmosphere of nitrogen, were placed 3-bromo-2-methoxypyridine (10.0 g, 53.2 mmol, 1.0 eq), cyclopropylboronic acid (5.5 g, 63.8 mmol, 1.2 eq), K₃PO₄ (22.6 g, 106.4 mmol, 2.0 eq), toluene (100 mL), water (10 mL), Pd(OAc)₂ (1.2 g, 5.3 mmol, 0.1 eq), tricyclohexylphosphane (3.0 g, 10.6 mmol, 0.2 eq). The resulting solution was stirred for 3 hours at 100° C. The resulting mixture was then quenched by the addition of water (100 mL), and extracted with ethyl acetate (2×300 mL). The combined organic layers was washed with brine (2×300 mL), dried over anhydrous Na₂SO₄. After filtration, the filtrate was concentrated under vacuum. The crude residue was purified onto a silica gel column and eluted with ethyl acetate/petroleum ether=1:4 to give 3-cyclopropyl-2-methoxypyridine as a light yellow oil (5.0 g, 63.0%). ¹H NMR (400 MHz, DMSO-d₆) δ 7.95 (dd, J=5.0, 1.8 Hz, 1H), 7.23 (dd, J=7.2, 2.0 Hz, 1H), 6.87 (dd, J=7.4, 4.8 Hz, 1H), 3.89 (s, 3H), 2.00 (tt, J=8.4, 5.4 Hz, 1H), 0.98-0.84 (m, 2H), 0.69-0.61 (m, 2H).

Synthesis of 5-bromo-3-cyclopropyl-2-methoxypyridine: Into an 8-mL sealed tube, were placed 3-cyclopropyl-2-methoxypyridine (5.0 g, 33.5 mmol, 1.0 eq), DCM (60 mL), Br₂ (5.9 g, 36.9 mmol, 1.1 eq). The resulting solution was stirred for overnight at 25° C. The resulting mixture was then quenched by the addition of water (100 mL), and extracted with dichloromethane (2×100 mL). The combined organic layers was washed with brine (2×100 mL), dried over anhydrous Na₂SO₄. After filtration, the filtrate was concentrated under vacuum. The crude product was purified by Prep-HPLC using the following conditions: Column, SunFire Prep C18 OBD Column, 50*250 mm 5 μm 10 nm; mobile phase, water (0.05% TFA) and CH₃CN (45% Phase B up to 65% in 7 min); Detector, UV 254/220 nm. Finally, 5-bromo-3-cyclopropyl-2-methoxypyridine was obtained as a light yellow oil (1.3 g, 17.0%). ¹H NMR (300 MHz, DMSO-d₆) δ 8.04 (d, J=2.4 Hz, 1H), 7.40 (d, J=2.4 Hz, 1H), 3.88 (s, 3H), 1.98 (tt, J=8.4, 5.4 Hz, 1H), 0.98-0.81 (m, 2H), 0.79-0.64 (m, 2H).

Synthesis of 5-cyclopropyl-6-methoxypyridine-3-carbaldehyde: Into a 50-mL 3-necked round-bottom flask, were placed 5-bromo-3-cyclopropyl-2-methoxypyridine (1.1 g, 4.8 mmol, 1.0 eq), THE (15 mL). This was followed by the addition of n-BuLi (2.5 M in hexanes, 2.9 mL) at −78° C. Then the mixture was stirred at −78° C. for 30 minutes. Then DMF (490 mg, 6.7 mmol, 1.4 eq) was added dropwise at −78° C. The mixture was stirred at −78° C. for 2 hours. The resulting mixture was then quenched by the addition of NH₄Cl (20 mL). The resulting solution was extracted with ethyl acetate (2×40 mL). The combined organic layers was washed with brine (2×40 mL), dried over anhydrous Na₂SO₄. After filtration, the filtrate was concentrated under vacuum. The crude residue was purified onto a silica gel column and eluted with ethyl acetate/petroleum ether=1:4 to give 5-cyclopropyl-6-methoxypyridine-3-carbaldehyde as a light yellow oil (550 mg, 64.4%). ¹H NMR (300 MHz, DMSO-d₆) δ 9.92 (s, 1H), 8.55 (d, J=2.1 Hz, 1H), 7.61 (d, J=2.1, 1H), 4.02 (s, 3H), 2.04 (ddd, J=13.8, 8.4, 5.4 Hz, 1H), 1.04-0.83 (m, 2H), 0.79-0.68 (m, 2H).

### Example INT_47 Preparation of 5-isopropoxy-6-(morpholin-4-yl)pyridine-3-carbaldehyde

Synthesis of 2-chloro-3-isopropoxypyridine: Into a 500-mL 3-necked round-bottom flask, were placed 2-chloropyridin-3-ol (15.0 g, 115.8 mmol, 1.0 eq), DMF (200 mL), K₂CO₃ (40.0 g, 289.5 mmol, 2.5 eq). This was followed by the addition of 2-iodopropane (23.6 g, 138.9 mmol, 1.2 eq) at 0° C. The mixture was stirred at 25° C. for 2 hours. The resulting mixture was then quenched by the addition of water (500 mL) and extracted with ethyl acetate (2×500 mL). The combined organic layers was washed with brine (2×500 mL), dried over anhydrous Na₂SO₄. After filtration, the filtrate was concentrated under vacuum. The crude residue was purified onto a silica gel column and eluted with ethyl acetate/petroleum ether=1:3 to give 2-chloro-3-isopropoxypyridine as a light yellow oil (16.0 g, 80.5%). ¹H NMR (400 MHz, DMSO-d₆) δ 7.95 (dd, J=4.6, 1.6 Hz, 1H), 7.59 (dd, J=8.4, 1.6 Hz, 1H), 7.36 (dd, J=8.2, 4.6 Hz, 1H), 4.72 (p, J=6.0 Hz, 1H), 1.30 (d, J=6.0 Hz, 6H).

Synthesis of 4-(3-isopropoxypyridin-2-yl)morpholine: Into a 500-mL 3-necked round-bottom flask, were placed 2-chloro-3-isopropoxypyridine (15.0 g, 87.4 mmol, 1.0 eq), morpholine (150 mL). The mixture was stirred at 130° C. for overnight. The resulting mixture was then quenched by the addition of water (300 mL), and extracted with ethyl acetate (2×300 mL). The combined organic layers was washed with brine (2×300 mL), dried over anhydrous Na₂SO₄. After filtration, the filtrate was concentrated under vacuum. The crude residue was purified onto a silica gel column and eluted with ethyl acetate/petroleum ether=1:2 to give 4-(3-isopropoxypyridin-2-yl)morpholine as a light yellow oil (16.0 g, 82.4%). ¹H NMR (400 MHz, DMSO-d₆) δ 7.79 (dd, J=4.8, 1.6 Hz, 1H), 7.23 (dd, J=7.8, 1.6 Hz, 1H), 6.86 (dd, J=8.0, 4.8 Hz, 1H), 4.59 (h, J=6.0 Hz, 1H), 3.74-3.67 (m, 4H), 3.33-3.26 (m, 4H), 1.27 (d, J=6.0 Hz, 6H).

Synthesis of 4-(5-bromo-3-isopropoxypyridin-2-yl)morpholine: Into a 500-mL 3-necked round-bottom flask, were placed 4-(3-isopropoxypyridin-2-yl)morpholine (10.0 g, 45.0 mmol, 1.0 eq), CCl₄ (100 mL), NBS (10.4 g, 58.5 mmol, 1.3 eq), AIBN (4.4 g, 27.0 mmol, 0.6 eq). The mixture was stirred at 80° C. for overnight. The resulting mixture was then quenched by the addition of water (200 mL), and extracted with dichloromethane (2×200 mL). The combined organic layers was washed with brine (2×200 mL), dried over anhydrous Na₂SO₄. After filtration, the filtrate was concentrated under vacuum. The crude residue was purified onto a silica gel column and eluted with ethyl acetate/petroleum ether=1:2 to give 4-(5-bromo-3-isopropoxypyridin-2-yl)morpholine as a light yellow oil (12.0 g, 88.6%). ¹H NMR (300 MHz, DMSO-d₆) δ 7.87 (d, J=2.1 Hz, 1H), 7.45 (d, J=2.1 Hz, 1H), 3.77-3.62 (m, 4H), 3.31-3.27 (m, 4H), 1.27 (d, J=6.0 Hz, 6H).

Synthesis of 5-isopropoxy-6-(morpholin-4-yl)pyridine-3-carbaldehyde: Into a 250 mL 3-necked round-bottom flask under nitrogen atmosphere, were placed 4-(5-bromo-3-isopropoxypyridin-2-yl)morpholine (6.0 g, 19.9 mmol, 1.0 eq), THE (60 mL). This was followed by the addition of n-BuLi (1.9 g, 29.9 mmol, 1.5 eq) at −78° C. The resulting mixture was stirred for 30 minutes at −78° C. To the above mixture was added DMF (2.0 g, 27.9 mmol, 1.4 eq) at −78° C. The resulting mixture was stirred for additional 30 minutes at −78° C. The resulting mixture was then quenched by the addition of water (200 mL), and extracted with ethyl acetate (2×100 mL). The combined organic layers was washed with brine (2×100 mL), dried over anhydrous Na₂SO₄. After filtration, the filtrate was concentrated under vacuum. The crude residue was purified onto a silica gel column and eluted with ethyl acetate/petroleum ether=1:1 to give 5-isopropoxy-6-(morpholin-4-yl)pyridine-3-carbaldehyde as a yellow solid (2.5 g, 50.1%). LC-MS (ESI, m/z) M+1: 251.

### Example INT_48 Preparation of 5-methoxy-6-[(3R)-3-methylmorpholin-4-yl]pyridine-3-carbaldehyde

Synthesis of (3R)-4-(3-methoxypyridin-2-yl)-3-methylmorpholine: Into a 500 mL round-bottom flask were added 2-bromo-3-methoxypyridine (10.0 g, 53.2 mmol, 1.0 eq), (3R)-3-methylmorpholine (8.1 g, 80.0 mmol, 1.5 eq), Cs₂CO₃ (10.1 g, 132.9 mmol, 2.5 eq) and DMSO (120 mL). The resulting mixture was stirred for 36 hours at 140° C. The reaction was quenched by the addition of water (50 mL), and extracted with EtOAc (3×200 mL). The combined organic layers were washed with brine (100 mL), dried over anhydrous Na₂SO₄. After filtration, the filtrate was concentrated under vacuum. The crude residue was applied onto a silica gel column and eluted with ethyl acetate/petroleum ether=1:5 to give (3R)-4-(3-methoxypyridin-2-yl)-3-methylmorpholine as an orange solid (4.1 g, 37.0%). LC-MS (ESI, m/z) M+1: 209.

Synthesis of (3R)-4-(5-bromo-3-methoxypyridin-2-yl)-3-methylmorpholine: Into a 50 mL round-bottom flask were added (3R)-4-(3-methoxypyridin-2-yl)-3-methylmorpholine (4.0 g, 19.2 mmol, 1.0 eq), NBS (6.8 g, 38.4 mmol, 2.0 eq) and DMF (10 mL). The resulting mixture was stirred for 4 hours at 80° C. The reaction was quenched by the addition of water (5 mL), and extracted with EtOAc (3×20 mL). The combined organic layers were washed with brine (20 mL), dried over anhydrous Na₂SO₄. After filtration, the filtrate was concentrated under vacuum. The crude residue was applied onto a silica gel column and eluted with ethyl acetate/petroleum ether=1:5 to give (3R)-4-(5-bromo-3-methoxypyridin-2-yl)-3-methylmorpholine as a red solid (1.8 g, 32.6%). ¹H NMR (300 MHz, Chloroform-d) δ 7.94 (d, J=2.0 Hz, 1H), 7.15 (d, J=2.0 Hz, 1H), 4.15 (qt, J=7.4, 3.6 Hz, 1H), 3.93 (dt, J=10.0, 3.7 Hz, 2H), 3.86 (s, 3H), 3.77 (ddd, J=11.3, 9.9, 3.0 Hz, 1H), 3.64 (dd, J=11.3, 3.3 Hz, 1H), 3.48 (ddd, J=13.2, 9.8, 3.4 Hz, 1H), 3.29 (dt, J=13.0, 3.2 Hz, 1H), 1.15 (d, J=6.6 Hz, 3H).

Synthesis of 5-methoxy-6-[(3R)-3-methylmorpholin-4-yl]pyridine-3-carbaldehyde: A solution of (3R)-4-(5-bromo-3-methoxypyridin-2-yl)-3-methylmorpholine (500 mg, 1.7 mmol, 1.0 eq) in THE (8 mL) was treated with n-BuLi in hexanes (133.9 mg, 2.1 mmol, 1.2 eq) for 1 hour at −78° C. This was followed by the addition of DMF (763 mg, 10.4 mmol, 6.0 eq) dropwise at −78° C. under nitrogen atmosphere. The resulting mixture was allowed to 25° C. The reaction was quenched by the addition of sat. NH₄Cl (10 mL) at 0° C. The resulting mixture was extracted with EtOAc (3×10 mL). The combined organic layers were washed with brine (10 mL), dried over anhydrous Na₂SO₄. After filtration, the filtrate was concentrated under vacuum. The crude residue was applied onto a silica gel column and eluted with ethyl acetate/petroleum ether=1:5 to give 5-methoxy-6-[(3R)-3-methylmorpholin-4-yl]pyridine-3-carbaldehyde as an orange solid (180 mg, 43.8%). ¹H NMR (300 MHz, Chloroform-d) δ 9.85 (s, 1H), 8.24 (d, J=1.8 Hz, 1H), 7.42 (d, J=1.8 Hz, 1H), 4.68 (d, J=7.5 Hz, 1H), 4.12 (d, J=13.4 Hz, 1H), 3.97 (d, J=12.1 Hz, 1H), 3.89 (s, 3H), 3.83 (dd, J=11.5, 3.1 Hz, 1H), 3.79-3.65 (m, 2H), 3.53 (ddd, J=13.4, 11.8, 3.4 Hz, 1H), 1.35 (d, J=6.8 Hz, 3H).

### Example INT_49 Preparation of 2-methyl-7-(morpholin-4-yl)furo[2,3-c]pyridine-4-carbaldehyde

Synthesis of 2-chloro-3-(prop-2-yn-1-yloxy)pyridine: Into a 2000-mL 4-necked round-bottom flask, were placed 2-chloropyridin-3-ol (50.0 g, 385.9 mmol, 1.0 eq), DMF (500 mL), K₂CO₃ (133.4 g, 964.9 mmol, 2.5 eq). This was followed by the addition of propargyl bromide (55.1 g, 463.2 mmol, 1.2 eq) at 0° C. The mixture was stirred at 25° C. for 2 hours. The resulting mixture was then quenched by the addition of water (1000 mL), and extracted with ethyl acetate (2×1500 mL). The combined organic layers was washed with brine (2×1500 mL), dried over anhydrous Na₂SO₄. After filtration, the filtrate was concentrated under vacuum. The crude residue was purified onto a silica gel column and eluted with ethyl acetate/petroleum ether=1:4 to give 2-chloro-3-(prop-2-yn-1-yloxy)pyridine as an off-white solid (60.0 g, 92.7%). ¹H NMR (300 MHz, DMSO-d₆) δ 8.03 (dd, J=4.5, 1.5 Hz, 1H), 7.65 (dd, J=8.1, 1.5 Hz, 1H), 7.44 (dd, J=8.1, 4.5 Hz, 1H), 5.00 (d, J=2.4 Hz, 2H), 3.69 (t, J=2.4 Hz, 1H).

Synthesis of 7-chloro-2-methylfuro[2,3-c]pyridine: Into a 1000-mL round-bottom flask, were placed 2-chloro-3-(prop-2-yn-1-yloxy)pyridine (32.0 g, 190.9 mmol, 1.0 eq), DMF (320 mL). The mixture was stirred at 150° C. for 3 days. The resulting mixture was then quenched by the addition of water (1000 mL), and extracted with ethyl acetate (2×1000 mL). The combined organic layers was washed with brine (2×1000 mL), dried over anhydrous Na₂SO₄. After filtration, the filtrate was concentrated under vacuum to give 7-chloro-2-methylfuro[2,3-c]pyridine as a light yellow solid (29.0 g, crude). ¹H NMR (300 MHz, Methanol-d₄) δ 8.06 (d, J=5.4 Hz, 1H), 7.50 (d, J=5.4 Hz, 1H), 6.65 (d, J=1.2 Hz, 1H), 2.55 (d, J=0.9 Hz, 3H).

Synthesis of 4-{2-methylfuro[2,3-c]pyridin-7-yl}morpholine: Into a 500-mL round-bottom flask, were placed 7-chloro-2-methylfuro[2,3-c]pyridine (12.0 g, 71.6 mmol, 1.0 eq), morpholine (120 mL). The mixture was stirred at 130° C. for overnight. The resulting mixture was then quenched by the addition of water (500 mL), and extracted with ethyl acetate (2×500 mL). The combined organic layers was washed with brine (2×500 mL), dried over anhydrous Na₂SO₄. After filtration, the filtrate was concentrated under vacuum. The crude residue was purified onto a silica gel column and eluted with ethyl acetate/petroleum ether=1:1 to give 4-{2-methylfuro[2,3-c]pyridin-7-yl}morpholine as a light yellow oil (2.0 g, 12.8%). ¹H NMR (400 MHz, DMSO-d₆) δ 7.85 (d, J=5.4 Hz, 1H), 6.97 (d, J=5.4 Hz, 1H), 6.58 (t, J=1.2 Hz, 1H), 3.79-3.64 (m, 8H), 2.47 (d, J=1.2 Hz, 3H).

Synthesis of 4-{4-bromo-2-methylfuro[2,3-c]pyridin-7-yl}morpholine: Into a 50-mL round-bottom flask, were placed 4-{2-methylfuro[2,3-c]pyridin-7-yl}morpholine (1.8 g, 8.2 mmol, 1.0 eq), DMF (20 mL), NBS (1.8 g, 9.9 mmol, 1.2 eq). The mixture was stirred at 80° C. for 4 hours. The resulting mixture was then quenched by the addition of water (50 mL), and extracted with ethyl acetate (2×50 mL). The combined organic layers was washed with brine (2×50 mL), dried over anhydrous Na₂SO₄. After filtration, the filtrate was concentrated under vacuum. The crude residue was purified onto a silica gel column and eluted with ethyl acetate/petroleum ether=1:1 to give 4-{4-bromo-2-methylfuro[2,3-c]pyridin-7-yl}morpholine as a light yellow solid (1.5 g, 61.2%). ¹H NMR (400 MHz, DMSO-d₆) δ 7.96 (s, 1H), 6.60 (q, J=1.0 Hz, 1H), 3.78-3.73 (m, 4H), 3.71-3.67 (m, 4H), 1.44 (s, 3H).

Synthesis of 2-methyl-7-(morpholin-4-yl)furo[2,3-c]pyridine-4-carbaldehyde: Into a 50-mL 3-necked round-bottom flask, were placed 4-{4-bromo-2-methylfuro[2,3-c]pyridin-7-yl}morpholine (1.3 g, 4.4 mmol, 1.0 eq), THE (15 mL). This was followed by the addition of n-BuLi (2.5 M in hexanes, 2.6 mL) at −78° C. Then the mixture was stirred at −78° C. for 30 minutes. After that, DMF (450 mg, 6.1 mmol, 1.4 eq) was added at −78° C. The mixture was stirred at −78° C. for 2 hours. The resulting mixture was then quenched by the addition of NH₄Cl (30 mL). The resulting solution was extracted with ethyl acetate (2×50 mL). The combined organic layers was washed with brine (2×50 mL), dried over anhydrous Na₂SO₄. After filtration, the filtrate was concentrated under vacuum. The crude residue was purified onto a silica gel column and eluted with ethyl acetate/petroleum ether=1:1 to give 2-methyl-7-(morpholin-4-yl)furo[2,3-c]pyridine-4-carbaldehyde as a light yellow solid (700 mg, 65.0%). ¹H NMR (400 MHz, DMSO-d₆) δ 9.88 (s, 1H), 8.39 (s, 1H), 7.06 (d, J=1.2 Hz, 1H), 4.02-3.98 (m, 4H), 3.76 (t, J=4.8 Hz, 4H), 2.52 (d, J=1.2 Hz, 3H).

### Example INT_51 Preparation of 5,6-dimethoxypyridine-3-carbaldehyde

Synthesis of 5-bromo-2,3-dimethoxypyridine: Into a 50-mL round-bottom flask, were placed 2,3-dimethoxypyridine (8.0 g, 57.5 mmol, 1.0 eq), Br₂ (10.1 g, 63.2 mmol, 1.1 eq), dichloromethane (80 mL) was stirred for 2 hours at 0° C. The resulting mixture was then quenched by the addition of water (50 mL), and extracted with ethyl acetate (50 mL). The organic phase was washed with water (50 mL×2) and brine (50 mL×2), and then dried over Na₂SO₄. After filtration, the resulting mixture was concentrated under vacuum. The crude residue was applied onto a silica gel column and eluted with ethyl acetate/petroleum ether=1:2 to give 5-bromo-2,3-dimethoxypyridine as an off-white oil (5.0 g, 40.0%). LC-MS (ESI, m/z) M+1: 218. ¹H NMR (300 MHz, Chloroform-d) δ 7.73 (d, J=2.1 Hz, 1H), 7.09 (d, J=2.1 Hz, 1H), 3.95 (s, 3H), 3.83 (s, 3H).

Synthesis of 5,6-dimethoxypyridine-3-carbaldehyde: Into a 250 mL 3-necked round-bottom flask, were placed 5-bromo-2,3-dimethoxypyridine (4.7 g, 21.6 mmol, 1.0 eq), THE (50 mL). This was followed by the addition of n-BuLi (1.1 eq) at −78° C. The resulting mixture was stirred for 30 minutes at −78° C. To the above mixture was added DMF (2.2 g, 30.2 mmol, 1.4 eq) at −78° C. The resulting mixture was stirred for additional 30 minutes at −78° C. The resulting mixture was then quenched by the addition of water (200 mL), and extracted with ethyl acetate (2×100 mL). The combined organic layers was washed with brine (2×100 mL). The mixture was dried over anhydrous Na₂SO₄. After filtration, the filtrate was concentrated under vacuum. The crude residue was purified onto a silica gel column and eluted with ethyl acetate/petroleum ether=1:1 to give 5,6-dimethoxypyridine-3-carbaldehyde as a light pink solid (2.7 g, 74.9%). LC-MS (ESI, m/z) M+1: 168. ¹H NMR (300 MHz, DMSO-d₆) δ 9.95 (s, 1H), 8.35 (d, J=1.8 Hz, 1H), 7.53 (d, J=1.8 Hz, 1H), 3.99 (s, 3H), 3.87 (s, 3H).

### Example INT_52 Preparation of 2,3-dimethoxyisonicotinaldehyde

Synthesis of 2,3-dimethoxyisonicotinaldehyde: Into a 250 mL round-bottom flask, a solution of 2,3-dimethoxypyridine (40 g, 18.3 mmol, 1.0 eq) in THE (100 mL) was treated with n-BuLi (1.3 g, 20.1 mmol, 1.1 eq) for 1 hour at −70° C. under nitrogen atmosphere. This was followed by the addition of DMF (1.6 g, 21.9 mmol, 1.2 eq) in portions at −70° C. The resulting mixture was stirred for 2 hours at −70° C. under N₂ atmosphere. The reaction was quenched by the addition of sat. NH₄Cl (30 mL) at 0° C. The resulting mixture was extracted with ethyl acetate (3×50 mL). The combined organic layers were washed with water (3×15 mL), dried over anhydrous Na₂SO₄. After filtration, the filtrate was concentrated under vacuum. The crude residue was purified onto a silica gel column and eluted with ethyl acetate/petroleum ether=1:1 to give 2,3-dimethoxyisonicotinaldehyde as a yellow solid (1.5 g, 49.2%). LC-MS (ESI, m/z) M+1: 168.

### Example INT_53 Preparation of 3-methoxy-2-morpholinoisonicotinaldehyde

Synthesis of 4-(3-methoxypyridin-2-yl)morpholine: Into a 250 mL round-bottom flask were added 2-chloro-3-methoxypyridine (10.0 g, 50.8 mmol, 1.0 eq), morpholine (9.6 g, 99.8 mmol, 2.0 eq) and K₂CO₃ (19 g, 150.8 mmol, 3.0 eq) in DMF (50 mL) at 25° C. The resulting mixture was stirred for 16 hours at 125° C. The resulting mixture was washed with water (3×100 mL). The aqueous layer was extracted with ethyl acetate (3×100 mL). The mixture was dried over anhydrous Na₂SO₄. After filtration, the filtrate was concentrated under vacuum. The crude residue was purified onto a silica gel column and eluted with ethyl acetate/petroleum ether=1:1 to give 4-(3-methoxypyridin-2-yl)morpholine as a yellow oil (50 g, 64.5%). LC-MS (ESI, m/z) M+1: 195.

Synthesis of 3-methoxy-2-morpholinoisonicotinaldehyde: Into a 250 mL round-bottom flask, a solution of 4-(3-methoxypyridin-2-yl)morpholine (5.0 g, 18.3 mmol, 1.0 eq) in THE (100 mL) was treated with n-BuLi (1.3 g, 20.1 mmol, 1.1 eq) for 1 hour at −70° C. under nitrogen atmosphere. This was followed by the addition of DMF (1.6 g, 21.9 mmol, 1.2 eq) in portions at −70° C. The resulting mixture was stirred for 4 hours at −70° C. under N₂ atmosphere. The reaction was quenched by the addition of sat. NH₄Cl (30 mL) at 0° C. The resulting mixture was extracted with ethyl acetate (3×50 mL). The combined organic layers were washed with water (3×15 mL), dried over anhydrous Na₂SO₄. After filtration, the filtrate was concentrated under vacuum. The crude residue was purified onto a silica gel column and eluted with ethyl acetate/petroleum ether=1:1 to give 3-methoxy-2-morpholinoisonicotinaldehyde as a yellow solid (2.0 g, 49.2%). LC-MS (ESI, m/z) M+1: 223.

### Example INT_55 Preparation of 3-fluoro-4-[(3R)-3-methylmorpholin-4-yl]benzaldehyde

Synthesis of 3-fluoro-4-[(3R)-3-methylmorpholin-4-yl]benzaldehyde: Into a 50 mL round-bottom flask, were placed 3,4-difluorobenzaldehyde (2.0 g, 14.1 mmol, 1.0 eq), (3R)-3-methylmorpholine (2.9 g, 28.1 mmol, 2.0 eq), K₂CO₃ (3.9 g, 28.1 mmol, 2.0 eq), DMSO (20 mL). The resulting solution was stirred for 12 hours at 130° C. The resulting mixture was then quenched by the addition of water (100 mL), and extracted with ethyl acetate (2×100 mL). The combined organic layers was washed with water (2×100 mL) and brine (2×100 mL), and then dried over anhydrous Na₂SO₄. After filtration, the filtrate was concentrated under vacuum. The crude residue was applied onto a silica gel column and eluted with ethyl acetate/petroleum ether=1:3 to give 3-fluoro-4-[(3R)-3-methylmorpholin-4-yl]benzaldehyde as a yellow oil (800 mg, 25.5%). LC-MS (ESI, m/z) M+1: 224.

### Example INT_56 Preparation of 4-cyclopropyl-3-methoxybenzaldehyde

Synthesis of 4-cyclopropyl-3-methoxybenzaldehyde: A solution mixture of 4-bromo-3-methoxybenzaldehyde (500 mg, 2.3 mmol, 1.0 eq), cyclopropylboronic acid (399 mg, 4.6 mmol, 2.0 eq), tricyclohexylphosphine (143 mg, 0.5 mmol, 0.2 eq), Pd(AcO)₂ (57 mg, 0.2 mmol, 0.1 eq) and K₃PO₄ (1480 mg, 6.9 mmol, 3.0 eq) in toluene (5 mL, 23 mmol, 10.0 eq) was stirred for 4 hours at 90° C. under nitrogen atmosphere. The reaction was quenched with water at 25° C. The resulting mixture was filtered the filter cake was washed with EtOAc (3×5 mL). The filtrate was concentrated under vacuum. The resulting mixture was extracted with EtOAc (3×10 mL). The combined organic layers were washed with brine (10 mL), dried over anhydrous Na₂SO₄. After filtration, the filtrate was concentrated under vacuum. The crude residue was purified by silica gel column chromatography, eluted with ethyl acetate/petroleum ether (1:5) to give 4-cyclopropyl-3-methoxybenzaldehyde as a light yellow solid (218 mg, 53.2%).

### Example INT_57 Preparation of 6-methoxy-5-methylnicotinaldehyde

Synthesis of 6-methoxy-5-methylnicotinaldehyde: A solution of 5-bromo-2-methoxy-3-methylpyridine (5 g, 24.7 mmol 1.0 eq) in THE (70 mL) was treated with n-BuLi (12 mL, 29.7 mmol, 1.2 eq) for 30 min at −78° C. under nitrogen atmosphere. This was followed by the addition of DMF (10 g, 6.0 eq) dropwise at −78° C. The reaction was quenched by the addition of sat. NH₄Cl (20 mL) at 0° C. The resulting mixture was extracted with EtOAc (3×40 mL). The combined organic layers were washed with brine (40 mL), dried over anhydrous Na₂SO₄. After filtration, the filtrate was concentrated under vacuum. The crude residue was purified by silica gel column chromatography, eluted with ethyl acetate/petroleum ether (1:5) to give 6-methoxy-5-methylnicotinaldehyde as a light yellow solid (3.3 g, 88.2%). LC-MS (ESI, m/z) M+1: 152.

### Example INT_58 Preparation of methyl 4-{2-oxo-7-azaspiro[3.5]nonan-7-yl}-2-[(3R,8S)-15-{[2-(trimethylsilyl)ethoxy]methyl}-2,5-dioxa-9,15,17-triazatetracyclo[8.7.0.0\hat{ }{3,8}.0\hat{ }{12,16}]heptadeca-1(10),11,13,16-tetraen-9-yl]benzoate

Synthesis of methyl 4-{2-oxo-7-azaspiro[3.5]nonan-7-yl}-2-[(3R,8S)-15-{[2-(trimethylsilyl)ethoxy]methyl}-2,5-dioxa-9,15,17-triazatetracyclo[8.7.0.0\hat{ }{3,8}.0\hat{ }{12,16}]heptadeca-1(10),11,13,16-tetraen-9-yl]benzoate: Into a 40 mL vial were added methyl 2-bromo-4-{2-oxo-7-azaspiro[3.5]nonan-7-yl}benzoate (1.0 g, 2.8 mmol, 1.0 eq), (3R,8S)-15-{[2-(trimethylsilyl)ethoxy]methyl}-2,5-dioxa-9,15,17-triazatetracyclo[8.7.0.0\hat{ }{3,8}.0\hat{ }{12,16}]heptadeca-1(10),11,13,16-tetraene (1.0 g, 2.8 mmol, 1.0 eq), Pd(dba)₂ (980 mg, 1.7 mmol, 0.6 eq), XantPhos (657 mg, 1.1 mmol, 0.4 eq), Cs₂CO₃ (1.8 g, 5.7 mmol, 2.0 eq) and toluene (10 mL). The resulting mixture was stirred for 6 hours at 80° C. under nitrogen atmosphere. The resulting mixture was filtered, the filter cake was washed with CH₂Cl₂ (3×10 mL). The filtrate was concentrated under vacuum. The resulting mixture was diluted with water (20 mL), and extracted with EtOAc (3×20 mL). The combined organic layers were washed with brine (10 mL), dried over anhydrous Na₂SO₄. After filtration, the filtrate was concentrated under vacuum. The crude residue was applied onto a silica gel column and eluted with ethyl acetate/petroleum ether=1:2 to give methyl 4-{2-oxo-7-azaspiro[3.5]nonan-7-yl}-2-[(3R,8S)-15-{[2-(trimethylsilyl)ethoxy]methyl}-2,5-dioxa-9,15,17-triazatetracyclo[8.7.0.0\hat{ }{3,8}.0\hat{ }{12,16}]heptadeca-1(10),11,13,16-tetraen-9-yl]benzoate as a brown solid (410 mg, 22.8%). LC-MS (ESI, m/z) M+1: 633.

### Example INT_59 (Method A) Preparation of (3S)-5-nitro-3-(oxan-4-yl)-3,4-dihydro-2H-1,4-benzoxazine-7-sulfonamide (assumed) and (3R)-5-nitro-3-(oxan-4-yl)-3,4-dihydro-2H-1,4-benzoxazine-7-sulfonamide (assumed)

Synthesis of 3-bromo-4-chloro-5-nitrobenzenesulfonamide: Into a 50-mL round-bottom flask, was placed 4-chloro-3-nitrobenzenesulfonamide (20.0 g, 84.5 mmol, 1.0 eq), H₂SO₄ (20 mL). This was followed by the addition of NBS (22.6 g, 126.8 mmol, 1.5 eq) in portions at 50° C. The resulting solution was stirred for 2 hours at 60° C. The resulting solution was diluted with 200 mL of cold water. The resulting solution was extracted with ethyl acetate (3×50 mL). The combined organic layers was washed with water (20 mL) and brine (20 mL). The crude residue was applied onto a silica gel column and eluted with ethyl acetate/petroleum ether=1:3 to give 3-bromo-4-chloro-5-nitrobenzenesulfonamide as a white solid (5.0 g, 18.7%). ¹H NMR (300 MHz, DMSO-d₆) δ 8.45 (s, 1H), 8.42 (s, 1H), 7.82 (s, 2H).

Synthesis of 2-amino-2-(oxan-4-yl)ethanol: Into a 100-mL round-bottom flask, was placed amino(oxan-4-yl)acetic acid (4.0 g, 25.1 mmol, 1.0 eq), THE (50 mL), LiAIH₄ (1.9 g, 50.3 mmol, 2.0 eq). The resulting solution was stirred for 3 hours at 60° C. The reaction was then quenched by the addition of Na₂SO₄. 10 H₂O (4.0 g). The resulting mixture was filtered and the filtrate was concentrated under vacuum. This resulted in 2-amino-2-(oxan-4-yl)ethanol as a white solid (6.0 g, crude).

Synthesis of 3-bromo-4-[[2-hydroxy-1-(oxan-4-yl)ethyl]amino]-5-nitrobenzenesulfonamide: Into a 100-mL round-bottom flask, was placed 3-bromo-4-chloro-5-nitrobenzenesulfonamide (5.0 g, 15.8 mmol, 1.0 eq), 2-amino-2-(oxan-4-yl)ethanol (5.0 g crude), CH₃CN (30 mL, 0.7 mmol), DIEA (6.1 g, 47.2 mmol, 3.0 eq). The resulting solution was stirred for 48 hours at 80° C. The resulting mixture was concentrated. The crude residue was applied onto a silica gel column and eluted with dichloromethane/methanol=10:1 to give 3-bromo-4-[[2-hydroxy-1-(oxan-4-yl) ethyl]amino]-5-nitrobenzenesulfonamide as a yellow solid (4.0 g, 59.7%). LC-MS (ESI, m/z) M+1: 424/426.

Synthesis of 5-nitro-3-(oxan-4-yl)-3,4-dihydro-2H-1,4-benzoxazine-7-sulfonamide: Into a 50-mL round-bottom flask purged and maintained with an inert atmosphere of nitrogen, was placed 3-bromo-4-[[2-hydroxy-1-(oxan-4-yl)ethyl]amino]-5-nitrobenzenesulfonamide (1.4 g, 3.3 mmol, 1.0 eq), dioxane (20 mL), Pd₂(dba)₃ (604 mg, 0.6 mmol, 0.2 eq), XantPhos (763 mg, 1.3 mmol, 0.4 eq), Cs₂CO₃ (3.2 g, 9.9 mmol, 3.0 eq). The resulting solution was stirred for 2 hours at 100° C. The resulting mixture was concentrated. The residue was applied onto a silica gel column and eluted with ethyl acetate/petroleum ether (1:1). Finally, 5-nitro-3-(oxan-4-yl)-3,4-dihydro-2H-1,4-benzoxazine-7-sulfonamide was obtained as a yellow solid (230 mg, 20.3%). LC-MS (ESI, m/z) M-1: 342.

Synthesis of (3S)-5-nitro-3-(oxan-4-yl)-3,4-dihydro-2H-1,4-benzoxazine-7-sulfonamide (assumed) and (3R)-5-nitro-3-(oxan-4-yl)-3,4-dihydro-2H-1,4-benzoxazine-7-sulfonamide (assumed): Into a 50-mL round-bottom flask, was placed 5-nitro-3-(oxan-4-yl)-3,4-dihydro-2H-1,4-benzoxazine-7-sulfonamide (650 mg) and DMF (10 m1). The sample was purified by SFC using the following conditions: Column: CHIRAL ART Cellulose-SJ, 3*25 cm, 5 um; Mobile Phase A: CO2, Mobile Phase B: MeOH; Flow rate: 90 mL/min; Gradient: isocratic 45% B; Detector, 220 nm. RT1(min): 4.41; RT₂(min): 5.26; Finally, (3S)-5-nitro-3-(oxan-4-yl)-3,4-dihydro-2H-1,4-benzoxazine-7-sulfonamide (assumed) was obtained as a yellow solid (250 mg). And (3R)-5-nitro-3-(oxan-4-yl)-3,4-dihydro-2H-1,4-benzoxazine-7-sulfonamide (assumed) was obtained as a yellow solid (240 mg). INT_M: RT₁=4.41 min in CHIRAL ART Cellulose-SJ, 3*25 cm, 5 um; Mobile Phase A: CO2, Mobile Phase B: MeOH(20 mM NH₃); Flow rate: 3.0 mL/min; Gradient: isocratic 45% B; Column Temperature: 35° C.; Back Pressure: 15 MPa; Wave Length: 220 nm; Sample Solvent: MeOH—HPLC; Injection Volume: 1 uL; INT_N: RT₁=5.26 min in CHIRAL ART Cellulose-SJ, 3*25 cm, 5 um; Mobile Phase A: CO2, Mobile Phase B: MeOH; Flow rate: 90 mL/min; Gradient: isocratic 45% B; Column Temperature: 35° C.; Back Pressure: 15 MPa; Wave Length: 220 nm; Sample Solvent: MeOH—HPLC; Injection Volume: 1 uL.

### Example INT_59 (Method B) Preparation of (3R)-5-nitro-3-(oxan-4-yl)-3,4-dihydro-2H-1,4-benzoxazine-7-sulfonamide

Synthesis of (4R)-4-benzyl-3-[2-(oxan-4-yl)acetyl]-1,3-oxazolidin-2-one: Into a 3000 mL 4-necked round-bottom flask were added oxan-4-ylacetic acid (100.0 g, 693.6 mmol, 1.0 eq), CH₂Cl₂ (1500 mL), (4R)-4-benzyl-1,3-oxazolidin-2-one (110.6 g, 624.3 mmol, 0.9 eq), EDC.HCl (172.9 g, 901.7 mmol, 1.3 eq), DMAP (169.5 g, 1387.3 mmol, 2.0 eq) at 10° C. The resulting mixture was stirred for 14 hours at 25° C. The residue was washed with HCl (1 M, 3×500 mL) and brine (500 mL), dried over anhydrous Na₂SO₄. After filtration, the filtrate was concentrated under vacuum. The crude residue was applied onto a silica gel column and eluted with ethyl acetate/petroleum ether=1:3 to give (4R)-4-benzyl-3-[2-(oxan-4-yl)acetyl]-1,3-oxazolidin-2-one as a white solid (150.0 g, 67.7%). ¹H NMR (400 MHz, Chloroform-d) δ 7.44-7.27 (m, 3H), 7.27-7.20 (m, 2H), 4.70 (ddt, J=10.4, 7.1, 3.3 Hz, 1H), 4.27-4.16 (m, 2H), 3.98 (ddd, J=11.4, 4.6, 2.3 Hz, 2H), 3.46 (tt, J=11.8, 2.4 Hz, 2H), 3.32 (dd, J=13.3, 3.4 Hz, 1H), 2.97 (dd, J=16.5, 6.6 Hz, 1H), 2.86 (dd, J=16.5, 7.0 Hz, 1H), 2.79 (dd, J=13.4, 9.6 Hz, 1H), 2.18 (ttt, J=10.9, 7.0, 3.8 Hz, 1H), 1.72 (dtt, J=11.1, 4.0, 2.0 Hz, 2H), 1.44 (tdd, J=13.2, 10.1, 6.2 Hz, 2H).

Synthesis of (4R)-3-[(2R)-2-azido-2-(oxan-4-yl)acetyl]-4-benzyl-1,3-oxazolidin-2-one: Into a 3 L 4-necked round-bottom flask were added (4R)-4-benzyl-3-[2-(oxan-4-yl)acetyl]-1,3-oxazolidin-2-one (100.0 g, 329.6 mmol, 1.0 eq) and tetrahydrofuran (1500 mL) at 25° C. under nitrogen atmosphere. To the above mixture was added KHMDS (1 M, 461 mL, 1.4 eq) dropwise over 20 minutes at −78° C. The resulting mixture was stirred for additional 45 minutes at −78° C. To the above mixture was added 2,4,6-triisopropylbenzenesulfonyl azide (122.4 g, 395.6 mmol, 1.2 eq) in tetrahydrofuran (500 mL) dropwise over 30 minutes at −78° C. The resulting mixture was stirred for additional 2 hours at −78° C. The reaction was quenched by the addition of HOAc (65 mL) at −78° C., this was followed by the addition of KOAc (100.0 g) at 25° C. The resulting mixture was stirred for additional 2 hours at 25° C. To the above mixture was added NaHCO₃ (sat. aq 500 mL) and extracted with Et₂O (3×300 mL). The combined organic layers were washed with brine (300 mL), dried over anhydrous Na₂SO₄. After filtration, the filtrate was concentrated under vacuum. The crude residue was applied onto a silica gel column and eluted with ethyl acetate/petroleum ether=1:5 to give (4R)-3-[(2R)-2-azido-2-(oxan-4-yl)acetyl]-4-benzyl-1,3-oxazolidin-2-one as a yellow oil (65.0 g, 37.2%).

Synthesis of (2R)-2-amino-2-(oxan-4-yl)ethanolas: Into a 1 L 3-necked round-bottom flask were added LiAIH₄ (18.6 g, 490.8 mmol, 4.0 eq) and tetrahydrofuran (500 mL) at 0° C. To the above mixture was added (4R)-3-[(2R)-2-azido-2-(oxan-4-yl)acetyl]-4-benzyl-1,3-oxazolidin-2-one (65.0 g, 122.7 mmol, 1.0 eq, 65%) in tetrahydrofuran (200 mL) dropwise over 30 minutes at 0° C. The resulting mixture was stirred for additional 14 hours at 25° C. The reaction was quenched by the addition of water (19 mL), 15% NaOH (19 mL) and water (57 mL) in this sequence at 0° C. The resulting mixture was diluted with tetrahydrofuran (300 mL). The resulting mixture was filtered, the filter cake was washed with tetrahydrofuran (3×100 mL), dried over anhydrous MgSO₄. After filtration, the filtrate was concentrated under vacuum to give (2R)-2-amino-2-(oxan-4-yl)ethanol as a yellow oil (28.0 g, crude).

Synthesis of 3-bromo-4-{[(1R)-2-hydroxy-1-(oxan-4-yl)ethyl]amino}-5-nitrobenzenesulfonamide: Into a 1000 mL round-bottom flask were added (2R)-2-amino-2-(oxan-4-yl)ethanol (28.0 g, crude (30%), 1.0 eq) and 3-bromo-4-chloro-5-nitrobenzenesulfonamide (18.6 g, 57.9 mmol, 1.0 eq), DIEA (24.9 g, 192.8 mmol, 3.3 eq) and N,N-dimethylformamide (300 mL) at 25° C. The resulting mixture was stirred for 14 hours at 80° C. The resulting mixture was diluted with water (1000 mL), and extracted with CH₂Cl₂ (3×300 mL). The combined organic layers were washed with brine (3×100 mL), dried over anhydrous Na₂SO₄. After filtration, the filtrate was concentrated under vacuum. The crude residue was applied onto a silica gel column and eluted with ethyl acetate/petroleum ether=1:1 to give 3-bromo-4-{[(1R)-2-hydroxy-1-(oxan-4-yl)ethyl]amino}-5-nitrobenzenesulfonamide as a yellow solid (12.0 g). ¹H NMR (300 MHz, DMSO-d₆) δ 8.27 (d, J=2.2 Hz, 1H), 8.15 (d, J=2.2 Hz, 1H), 7.49 (s, 2H), 6.73 (d, J=10.3 Hz, 1H), 4.92 (t, J=4.9 Hz, 1H), 3.91-3.81 (m, 2H), 3.73-3.65 (m, 1H), 3.60-3.48 (m, 1H), 3.53-3.41 (m, 1H), 3.33-3.17 (m, 1H), 1.58 (t, J=15.0 Hz, 2H), 1.32 (tq, J=13.2, 7.4, 6.6 Hz, 3H).

Synthesis of (3R)-5-nitro-3-(oxan-4-yl)-3,4-dihydro-2H-1,4-benzoxazine-7-sulfonamide: Into a 500 mL round-bottom flask were added 3-bromo-4-{[(1R)-2-hydroxy-1-(oxan-4-yl)ethyl]amino}-5-nitrobenzenesulfonamide (12.0 g, 24.0 mmol, 1.0 eq, 85%), (1R,2R)—N¹,N²-dimethylcyclohexane-1,2-diamine (2.1 g, 14.4 mmol, 0.6 eq), N,N-dimethylformamide (200 mL), Cul (2.8 g, 14.4 mmol, 0.6 eq), KOAc (7.1 g, 72.1 mmol, 3.0 eq). The resulting mixture was stirred for 14 hours at 100° C. under nitrogen atmosphere. The reaction was quenched by the addition of water (400 mL). The resulting mixture was extracted with CH₂Cl₂ (3×200 mL). The combined organic layers were washed with brine (3×100 mL), dried over anhydrous Na₂SO₄. After filtration, the filtrate was concentrated under vacuum. The crude residue was applied onto a silica gel column and eluted with ethyl acetate/petroleum ether=2:1 to give (3R)-5-nitro-3-(oxan-4-yl)-3,4-dihydro-2H-1,4-benzoxazine-7-sulfonamide as a yellow solid (5.7 g, 51.8%). ¹H NMR (300 MHz, DMSO-d₆) δ 8.80 (d, J=4.4 Hz, 1H), 8.10 (d, J=2.1 Hz, 1H), 7.36-7.29 (m, 3H), 4.40 (dd, J=11.3, 2.7 Hz, 1H), 4.13-3.97 (m, 1H), 3.88 (dd, J=11.7, 4.0 Hz, 2H), 3.54 (dd, J=7.5, 4.1 Hz, 1H), 3.24 (tdd, J=11.8, 4.1, 2.1 Hz, 2H), 1.89-1.72 (m, 1H), 1.63 (t, J=14.2 Hz, 2H), 1.39-1.20 (m, 2H).

### Example INT_60 Preparation of (S)-3-((1r,4S)-4-hydroxy-4-methylcyclohexyl)-5-nitro-3,4-dihydro-2H-benzo[b][1,4]oxazine-7-sulfonamide

Synthesis of (S)-2-((tert-butoxycarbonyl)amino)-2-(4-hydroxyphenyl)acetic acid: Into a 5 L 4-necked round-bottom flask were added (S)-2-amino-2-(4-hydroxyphenyl)acetic acid (200.0 g, 1.2 mol, 1.0 eq) and NaOH(10 M) (400 mL) at 0° C. The resulting mixture was stirred for 0.5 hour at 0° C. under N₂ atmosphere. To the above mixture was added di-tert-butyl dicarbonate (339.4 g, 1.5 mol, 1.3 eq) and THE (2000 mL) dropwise over 0.5 hour at 0° C. The resulting mixture was stirred for additional 16 hours at 25° C. The mixture was acidified to pH=2 with HCl (6 M) under 0° C. The resulting mixture was extracted with Et₂O (3×1000 mL). The combined organic layers were washed with brine (2×500 mL), dried over anhydrous Na₂SO₄. After filtration, the filtrate was concentrated under vacuum. The crude residue was purified onto a silica gel column and eluted with ethyl acetate/petroleum ether=1:5 to give (S)-2-((tert-butoxycarbonyl)amino)-2-(4-hydroxyphenyl)acetic acid as a white solid (300.0 g, 93.8%). LC-MS (ESI, m/z) M+1: 268.

Synthesis of (S)-4-(1-amino-2-hydroxyethyl)phenol: Into a 5 L round-bottom flask, to a stirred solution of (S)-2-((tert-butoxycarbonyl)amino)-2-(4-hydroxyphenyl)acetic acid (250.0 g, 935.3 mmol, 1.0 eq) in THE (2000 mL) was added BH₃-THF (4000 mL) dropwise at 0° C. under N₂ atmosphere. The resulting mixture was stirred for 7 hours at 25° C. The reaction was quenched by the addition of MeOH (1000 mL) at 0° C. The resulting mixture was concentrated under vacuum. The crude residue was purified onto a silica gel column and eluted with ethyl acetate/petroleum ether=1:1 to give (S)-4-(1-amino-2-hydroxyethyl) phenol as a white solid (100.0 g, 69.8%). LC-MS (ESI, m/z) M+1: 154.

Synthesis of tert-butyl (S)-(2-hydroxy-1-(4-hydroxyphenyl)ethyl)carbamate: Into a 5 L round-bottom flask, to a stirred solution of (S)-4-(1-amino-2-hydroxyethyl)phenol (100.0 g, 652.8 mmol, 1.0 eq) in MeOH (1500 mL) was added di-tert-butyl dicarbonate (185.2 g, 848.6 mmol, 1.3 eq) dropwise at 0° C. under N₂ atmosphere. The resulting mixture was stirred for 16 hours at 25° C. The resulting mixture was concentrated under vacuum. The crude residue was purified onto a silica gel column and eluted with ethyl acetate/petroleum ether=1:5 to give tert-butyl tert-butyl (S)-(2-hydroxy-1-(4-hydroxyphenyl)ethyl)carbamate as a white solid (120.0 g, 72.5%). LC-MS (ESI, m/z) M+1: 254.

Synthesis of tert-butyl (S)-4-(4-hydroxyphenyl)-2,2-dimethyloxazolidine-3-carboxylate: Into a 5 L round-bottom flask, a solution of tert-butyl (S)-(2-hydroxy-1-(4-hydroxyphenyl)ethyl)carbamate (120.0 g, 473.7 mmol, 1.0 eq) in CH₂Cl₂ (1500 mL) was treated with 2,2-dimethoxypropane (98.6 g, 947.5 mmol, 2.0 eq) for 10 min at 0° C. under nitrogen atmosphere. This was followed by the addition of TsOH (16.3 g, 94.7 mmol, 0.2 eq) dropwise at 0° C. The resulting mixture was stirred for 3 hours at 25° C. The resulting mixture was washed with H₂O (3×100 mL), dried over Na₂SO₄ and concentrated in vacuo. The crude residue was applied onto a silica gel column and eluted with ethyl acetate/petroleum ether=1:1 to give tert-butyl (S)-4-(4-hydroxyphenyl)-2,2-dimethyloxazolidine-3-carboxylate as a white solid (50.1 g, 35.9%). LC-MS (ESI, m/z) M+1: 294.

Synthesis of tert-butyl (S)-4-(4-hydroxycyclohexyl)-2,2-dimethyloxazolidine-3-carboxylate: Into a 2 L round-bottom flask, to a stirred solution of tert-butyl (S)-4-(4-hydroxyphenyl)-2,2-dimethyloxazolidine-3-carboxylate (50.0 g, 170.4 mmol, 1.0 eq) in i-PrOH (800 mL) was added Adams's catalyst (3.8 g, 17.0 mmol, 0.1 eq) in portions at 25° C. under H₂ atmosphere. The resulting mixture was stirred for 24 hours at 50° C. under H₂ (5 atm) atmosphere. The resulting mixture was filtered, the filter cake was washed with i-PrOH (3×300 mL). The filtrate was concentrated under vacuum. The crude residue was applied onto a silica gel column and eluted with ethyl acetate/petroleum ether=1:1 to give tert-butyl (S)-4-(4-hydroxycyclohexyl)-2,2-dimethyloxazolidine-3-carboxylate as a yellow oil (25.2 g, 48.9%). LC-MS (ESI, m/z) M+1: 300.

Synthesis of tert-butyl (S)-2,2-dimethyl-4-(4-oxocyclohexyl)oxazolidine-3-carboxylate: Into a 2 L round-bottom flask, to a stirred solution of tert-butyl (S)-4-(4-hydroxycyclohexyl)-2,2-dimethyloxazolidine-3-carboxylate (25.0 g, 83.4 mmol, 1.0 eq) in CH₂Cl₂ (500 mL) was added DMP (106.2 g, 250.4 mmol, 3.0 eq) in portions at 0° C. The resulting mixture was stirred for 16 hours at 25° C. The resulting mixture was washed with water (3×100 mL). The aqueous layer was extracted with ethyl acetate (2×100 mL). The combined organic layers were dried over anhydrous Na₂SO₄. After filtration, the filtrate was concentrated under vacuum. The crude residue was applied onto a silica gel column and eluted with ethyl acetate/petroleum ether=1:1 to give tert-butyl tert-butyl (S)-2,2-dimethyl-4-(4-oxocyclohexyl)oxazolidine-3-carboxylate as a yellow solid (12.0 g, 48.3%). LC-MS (ESI, m/z) M+1: 298.

Synthesis of tert-butyl (S)-4-(4-hydroxy-4-methylcyclohexyl)-2,2-dimethyloxazolidine-3-carboxylate: Into a 500 mL round-bottom flask, to a stirred solution of tert-butyl (S)-2,2-dimethyl-4-(4-oxocyclohexyl)oxazolidine-3-carboxylate (12.0 g, 40.3 mmol, 1.0 eq) in tetrahydrofuran (200 mL) was added methyllithium (2.6 g, 121.0 mmol, 3.0 eq) in THE dropwise at −78° C. under N₂ atmosphere. The resulting mixture was stirred for 3 hours at −78° C. under N₂ atmosphere. The reaction was quenched by the addition of sat. NH₄Cl (100 mL) at 0° C. The resulting mixture was extracted with ethyl acetate (3×100 mL). The combined organic layers were washed with brine (3×50 mL), dried over anhydrous Na₂SO₄. After filtration, the filtrate was concentrated under vacuum. The crude residue was applied onto a silica gel column and eluted with ethyl acetate/petroleum ether=1:1 to give tert-butyl (S)-4-(4-hydroxy-4-methylcyclohexyl)-2,2-dimethyloxazolidine-3-carboxylate as a yellow solid (7.0 g, 55.3%). LC-MS (ESI, m/z) M+1: 314.

Synthesis of (S)-4-(1-amino-2-hydroxyethyl)-1-methylcyclohexan-1-ol: Into a 500 mL round-bottom flask, to a stirred solution of tert-butyl (S)-4-(4-hydroxy-4-methylcyclohexyl)-2,2-dimethyloxazolidine-3-carboxylate (6.0 g, 19.1 mmol, 1.0 eq) in ethyl acetate (20 mL) was added HCl/1,4-dioxane (100 mL) dropwise at 0° C. The resulting mixture was stirred for 5 hours at 30° C. The resulting mixture was concentrated under vacuum, to give (S)-4-(1-amino-2-hydroxyethyl)-1-methylcyclohexan-1-ol as a yellow oil (2.3 g, 69.3%). LC-MS (ESI, m/z) M+1: 174.

Synthesis of 3-bromo-4-(((S)-2-hydroxy-1-((1r,4S)-4-hydroxy-4-methylcyclohexyl)ethyl)amino)-5-nitrobenzenesulfonamide and 3-bromo-4-(((S)-2-hydroxy-1-((1s,4R)-4-hydroxy-4-methylcyclohexyl)ethyl)amino)-5-nitrobenzenesulfonamide: Into a 250 mL round-bottom flask, to a stirred solution of (S)-4-(1-amino-2-hydroxyethyl)-1-methylcyclohexan-1-ol (2.6 g, 15.0 mmol, 1.0 eq) and 3-bromo-4-fluoro-5-nitrobenzenesulfonamide (6.7 g, 22.5 mmol, 1.5 eq) in DMF (50 mL) was added K₂CO₃ (8.3 g, 60.0 mmol, 4.0 eq) and DIEA (7.7 g, 60.0 mmol, 4.0 eq) dropwise at 25° C. The resulting mixture was stirred for 12 hours at 30° C. The resulting mixture was diluted with ethyl acetate (100 mL). The resulting mixture was washed with H₂O (3×50 mL). The water was extracted with ethyl acetate (3×50 mL). The combined organic layers were washed with brine (3×50 mL), dried over anhydrous Na₂SO₄. After filtration, the filtrate was concentrated under vacuum. The crude residue was applied onto a silica gel column and eluted with ethyl acetate/petroleum ether=1:5 to give 3-bromo-4-(((S)-2-hydroxy-1-((1r,4S)-4-hydroxy-4-methylcyclohexyl)ethyl)amino)-5-nitrobenzenesulfonamide as a yellow solid (1.0 g, 14.3%) and 3-bromo-4-(((S)-2-hydroxy-1-((1s,4R)-4-hydroxy-4-methylcyclohexyl)ethyl)amino)-5-nitrobenzenesulfonamide as a yellow solid (500 mg, 7.3%). LC-MS (ESI, m/z) M+1: 452. A-¹H NMR (300 MHz, DMSO-d₆) δ 8.26 (d, J=2.2 Hz, 1H), 8.14 (d, J=2.4 Hz, 1H), 7.50 (s, 1H), 6.76 (d, J=10.2 Hz, 2H), 4.90 (s, 1H), 3.93 (s, 1H), 3.69 (s, 1H), 3.55 (d, J=11.4 Hz, 1H), 3.52 (s, 1H), 1.63-1.36 (m, 6H), 1.36-1.11 (m, 3H), 1.07 (s, 3H). B-¹H NMR (300 MHz, DMSO-d₆) δ 8.26 (d, J=2.1 Hz, 1H), 8.14 (d, J=2.1 Hz, 1H), 7.52-7.45 (m, 2H), 6.70 (d, J=10.5 Hz, 1H), 4.88 (t, J=4.8 Hz, 1H), 4.25 (s, 1H), 4.04 (q, J=7.2 Hz, 1H), 3.71 (s, 1H), 3.52 (q, J=5.1 Hz, 2H), 3.33 (s, 1H), 2.90 (s, 1H), 2.74 (s, 1H), 1.99 (s, 1H), 1.62 (d, J=0.6 Hz, 1H), 1.58-1.35 (m, 1H), 1.38-1.22 (m, 1H), 1.18 (t, J=7.2 Hz, 1H), 1.13 (s, 1H), 1.11-0.96 (m, 2H).

Synthesis of (S)-3-((1r,4S)-4-hydroxy-4-methylcyclohexyl)-5-nitro-3,4-dihydro-2H-benzo[b][1,4]oxazine-7-sulfonamide: To a stirred solution of 3-bromo-4-(((S)-2-hydroxy-1-((1r,4S)-4-hydroxy-4-methylcyclohexyl)ethyl)amino)-5-nitrobenzenesulfonamide (1.0 g, 2.2 mmol, 1.0 eq) in dioxane (50 mL) were added Xantphos Pd 4G (0.43 g, 0.4 mmol, 0.2 eq) and Cs₂CO₃ (1.4 g, 4.4 mmol, 2.0 eq) in portions at 25° C. under N₂ atmosphere. The resulting mixture was stirred for 14 hours at 100° C. under N₂ atmosphere. The precipitated solids were collected by filtration and washed with ethyl acetate (3×50 mL). The resulting mixture was concentrated under vacuum. The crude residue was applied onto a silica gel column and eluted with ethyl acetate/petroleum ether=1:5 to give (S)-3-((1r,4S)-4-hydroxy-4-methylcyclohexyl)-5-nitro-3,4-dihydro-2H-benzo[b][1,4]oxazine-7-sulfonamide as a yellow solid (200 mg, 24.3%). LC-MS (ESI, m/z) M+1: 372.

### Example INT_61 Preparation of 4-{2-[(2S)-2-(2-isopropoxyphenyl)-4-{[5-methoxy-6-(morpholin-4-yl)pyridin-3-yl]methyl}piperazin-1-yl]-7-azaspiro[3.5]nonan-7-yl}-2-[(3R,8S)-15-{[2-(trimethylsilyl)ethoxy]methyl}-2,5-dioxa-9,15,17-triazatetracyclo[8.7.0.0\hat{ }{3,8}.0\hat{ }{12,16}]heptadeca-1(10),11,13,16-tetraen-9-yl]benzoic acid (assumed)

Synthesis of 4-(5-{[(3S)-3-(2-isopropoxyphenyl)piperazin-1-yl]methyl}-3-methoxypyridin-2-yl)morpholine (assumed): Into a 100 mL round-bottom flask were added 5-methoxy-6-(morpholin-4-yl)pyridine-3-carbaldehyde (500 mg, 2.2 mmol, 1.0 eq), (2S)-2-(2-isopropoxyphenyl)piperazine (assumed) (496 mg, 2.2 mmol, 1.0 eq), NaBH₃CN (707 mg, 11.2 mmol, 5.0 eq), ZnCl₂ (1533 mg, 11.2 mmol, 5.0 eq) and MeOH (10 mL). The resulting mixture was stirred for 2 hours at 25° C. The reaction was quenched by the addition of water (10 mL) and extracted with CH₂Cl₂ (3×10 mL). The combined organic layers were washed with brine (10 mL), dried over anhydrous Na₂SO₄. After filtration, the filtrate was concentrated under vacuum. The crude residue was applied onto a silica gel column and eluted with ethyl acetate/petroleum ether=1:1 to give 4-(5-{[(3S)-3-(2-isopropoxyphenyl)piperazin-1-yl]methyl}-3-methoxypyridin-2-yl)morpholine (assumed) as a greenish solid (620 mg, 64.6%). LC-MS (ESI, m/z) M+1: 427.

Synthesis of methyl 4-{2-[(2S)-2-(2-isopropoxyphenyl)-4-{[5-methoxy-6-(morpholin-4-yl)pyridin-3-yl]methyl}piperazin-1-yl]-7-azaspiro[3.5]nonan-7-yl}-2-[(3R,8S)-15-{[2-(trimethylsilyl)ethoxy]methyl}-2,5-dioxa-9,15,17-triazatetracyclo[8.7.0.0\hat{ }{3,8}.0\hat{ }{12,16}]heptadeca-1(10),11,13,16-tetraen-9-yl]benzoate (assumed): Into a 50 mL round-bottom flask were added 4-(5-{[(3S)-3-(2-isopropoxyphenyl)piperazin-1-yl]methyl}-3-methoxypyridin-2-yl)morpholine (assumed) (400 mg, 0.9 mmol, 1.0 eq), methyl 4-{2-oxo-7-azaspiro[3.5]nonan-7-yl}-2-[(3R,8S)-15-{[2-(trimethylsilyl)ethoxy]methyl}-2,5-dioxa-9,15,17-triazatetracyclo[8.7.0.0\hat{ }{3,8}.0\hat{ }{12,16}]heptadeca-1(10),11,13,16-tetraen-9-yl]benzoate (593 mg, 0.9 mmol, 1.0 eq), NaBH₃CN (294 mg, 4.7 mmol, 5.0 eq), ZnCl₂ (639 mg, 4.7 mmol, 5.0 eq) and MeOH (8 mL). The resulting mixture was stirred for 4 hours at 50° C. The reaction was quenched by the addition of water (10 mL) and extracted with CH₂Cl₂ (3×10 mL). The combined organic layers were washed with brine (3×10 mL), dried over anhydrous Na₂SO₄. After filtration, the filtrate was concentrated under vacuum. The crude residue was applied onto a silica gel column and eluted with ethyl acetate/petroleum ether=1:1 to give methyl 4-{2-[(2S)-2-(2-isopropoxyphenyl)-4-{[5-methoxy-6-(morpholin-4-yl)pyridin-3-yl]methyl}piperazin-1-yl]-7-azaspiro[3.5]nonan-7-yl}-2-[(3R,8S)-15-{[2-(trimethylsilyl)ethoxy]methyl}-2,5-dioxa-9,15,17-triazatetracyclo[8.7.0.0\hat{ }{3,8}.0\hat{ }{12,16}]heptadeca-1(10),11,13,16-tetraen-9-yl]benzoate (assumed) as a greenish solid (560 mg, 57.2%). LC-MS (ESI, m/z) M+1: 1043.

Synthesis of 4-{2-[(2S)-2-(2-isopropoxyphenyl)-4-{[5-methoxy-6-(morpholin-4-yl)pyridin-3-yl]methyl}piperazin-1-yl]-7-azaspiro[3.5]nonan-7-yl}-2-[(3R,8S)-15-{[2-(trimethylsilyl)ethoxy]methyl}-2,5-dioxa-9,15,17-triazatetracyclo[8.7.0.0\hat{ }{3,8}.0\hat{ }{12,16}]heptadeca-1(10),11,13,16-tetraen-9-yl]benzoic acid (assumed): Into a 50 mL round-bottom flask were added methyl 4-{2-[(2S)-2-(2-isopropoxyphenyl)-4-{[5-methoxy-6-(morpholin-4-yl)pyridin-3-yl]methyl}piperazin-1-yl]-7-azaspiro[3.5]nonan-7-yl}-2-[(3R,8S)-15-{[2-(trimethylsilyl)ethoxy]methyl}-2,5-dioxa-9,15,17-triazatetracyclo[8.7.0.0\hat{ }{3,8}.0\hat{ }{12,16}]heptadeca-1(10),11,13,16-tetraen-9-yl]benzoate (assumed) (500 mg, 0.5 mmol, 1.0 eq), NaOH(4 M, 2 mL) and dioxane/MeOH (5 mL/5 mL). The resulting mixture was stirred for overnight at 50° C. The resulting mixture was concentrated under vacuum. The residue was dissolved in CH₂Cl₂ (10 mL). The resulting mixture was diluted with water (10 mL) and extracted with CH₂Cl₂ (3×10 mL). The combined organic layers were washed with brine (10 mL), dried over anhydrous Na₂SO₄. After filtration, the filtrate was concentrated under vacuum to give 4-{2-[(2S)-2-(2-isopropoxyphenyl)-4-{[5-methoxy-6-(morpholin-4-yl)pyridin-3-yl]methyl}piperazin-1-yl]-7-azaspiro[3.5]nonan-7-yl}-2-[(3R,8S)-15-{[2-(trimethylsilyl)ethoxy]methyl}-2,5-dioxa-9,15,17-triazatetracyclo[8.7.0.0\hat{ }{3,8}.0\hat{ }{12,16}]heptadeca-1(10),11,13,16-tetraen-9-yl]benzoic acid (assumed) as a yellow green solid (410 mg, 83.1%). LC-MS (ESI, m/z) M+1: 1029.

### Example INT_62 Preparation of 4-{1-methyl-2-oxo-7-azaspiro[3.5]nonan-7-yl}-N-[3-nitro-4-({[(1r,4r)-4-hydroxy-4-methylcyclohexyl]methyl}amino)benzenesulfonyl]-2-[(3R,8S)-15-{[2-(trimethylsilyl)ethoxy]methyl}-2,5-dioxa-9,15,17-triazatetracyclo[8.7.0.0\hat{ }{3,8}.0\hat{ }{12,16}]heptadeca-1(10),11,13,16-tetraen-9-yl]benzamide

Synthesis of Synthesis of tert-butyl 4-ethylidenepiperidine-1-carboxylate: Into a 1000 mL round-bottom flask, were placed benzen-2-ylium-1-yl(ethyl)diphenylphosphanium (61.2 g, 210.8 mmol, 1.4 eq), tetrahydrofuran (500 mL). To the above mixture was added butyllithium (14.5 g, 225.8 mmol, 1.5 eq) in portions at −78° C. The reaction mixture was stirred for additional 1 hour at −78° C. To the above mixture was added tert-butyl 4-oxopiperidine-1-carboxylate (30.0 g, 150.6 mmol, 1.0 eq). The reaction mixture was stirred for overnight at 25° C. The resulting mixture was quenched by the addition of aqueous NH₄Cl and extracted with EtOAc (2×500 mL). The combined organic layer was washed with brine (500 mL), dried over anhydrous Na₂SO₄. After filtration, the filtrate was concentrated under vacuum. The crude residue was applied onto a silica gel column and eluted with ethyl acetate/petroleum ether=1:2 to give tert-butyl 4-ethylidenepiperidine-1-carboxylate as a colorless oil (25.0 g, 78.6%). ¹H NMR (300 MHz, Chloroform-d) δ 5.29 (dtd, J=8.1, 6.9, 6.0, 2.1 Hz, 1H), 3.45-3.35 (m, 4H), 2.27-2.12 (m, 4H), 1.61 (d, J=6.6 Hz, 3H), 1.49 (s, 9H).

Synthesis of tert-butyl 1,1-dichloro-3-methyl-2-oxo-7-azaspiro[3.5]nonane-7-carboxylate: Into a 500 mL round-bottom flask purged and maintained with an inert atmosphere of nitrogen, were placed tert-butyl 4-ethylidenepiperidine-1-carboxylate (8.0 g, 37.9 mmol, 1.0 eq) and zinc-copper couple (35.0 g, 275.6 mmol, 7.0 eq), DME (200 mL). After that, trichloroacetyl chloride (22.7 g, 125.0 mmol, 3.3 eq) was added dropwise with stirring at 0° C. The resulting mixture was stirred for overnight at 25° C. The resulting mixture was filtered through a Celite pad and concentrated under vacuum to give tert-butyl 1,1-dichloro-3-methyl-2-oxo-7-azaspiro[3.5]nonane-7-carboxylate as a colorless oil (12.0 g, 98.4%). LC-MS (ESI, m/z) M-t-Bu+CH₃CN: 307.

Synthesis of tert-butyl 1-methyl-2-oxo-7-azaspiro[3.5]nonane-7-carboxylate: Into a 250 mL 3-necked round-bottom flask, were placed tert-butyl 1,1-dichloro-3-methyl-2-oxo-7-azaspiro[3.5]nonane-7-carboxylate (8.0 g, 24.8 mmol, 1.0 eq), methanol (70 mL), saturated aqueous NH₄Cl (70 mL), Zn (9.7 g, 149.0 mmol, 6.0 eq). The reaction mixture was stirred for 16 hours at 25° C. under nitrogen atmosphere, filtered through a Celite pad and concentrated under vacuum. The crude residue was applied onto a silica gel column and eluted with ethyl acetate/petroleum ether=1:4 to give tert-butyl 1-methyl-2-oxo-7-azaspiro[3.5]nonane-7-carboxylate as a brown oil (4.0 g, 63.6%). ¹H NMR (300 MHz, Chloroform-d) δ 3.98 (dt, J=13.2, 4.5 Hz, 2H), 3.06-2.84 (m, 3H), 2.82-2.69 (m, 2H), 1.95-1.79 (m, 1H), 1.77-1.57 (m, 3H), 1.48 (s, 9H), 1.07 (d, J=7.5 Hz, 3H).

Synthesis of 1-methyl-7-azaspiro[3.5]nonan-2-one: Into a 250 mL round-bottom flask, were placed tert-butyl 1-methyl-2-oxo-7-azaspiro[3.5]nonane-7-carboxylate (4.0 g, 15.8 mmol, 1.0 eq), HCl/1,4-dioxane (50 mL). The reaction mixture was stirred for 1 hour at 25° C. The resulting mixture was concentrated under vacuum. The resulting mixture was diluted with saturated aqueous NaHCO₃ ((100 mL) and extracted with CH₂Cl₂ (2×200 mL). The combined organic layer was washed with brine (200 mL), dried over anhydrous Na₂SO₄. After filtration, the filtrate was concentrated under vacuum to give 1-methyl-7-azaspiro[3.5]nonan-2-one as a light yellow oil (2.2 g, 90.9%). ¹H NMR (300 MHz, DMSO-d₆) δ 9.14 (s, 1H), 3.36 (s, 2H), 3.26-3.04 (m, 3H), 2.97 (ddd, J=17.1, 3.1, 1.2 Hz, 1H), 2.81 (dd, J=17.0, 2.0 Hz, 1H), 2.03 (ddd, J=14.0, 11.5, 4.1 Hz, 1H), 1.88-1.75 (m, 2H), 1.57 (d, J=14.2 Hz, 1H), 0.97 (d, J=7.5 Hz, 3H).

Synthesis of methyl 2-bromo-4-{1-methyl-2-oxo-7-azaspiro[3.5]nonan-7-yl}benzoate: Into a 250 mL 3-necked round-bottom flask, were placed 1-methyl-7-azaspiro[3.5]nonan-2-one (2.0 g, 13.0 mmol, 1.0 eq), DMSO (100 mL), methyl 2-bromo-4-fluorobenzoate (4.9 g, 14.4 mmol, 1.1 eq), Na₂CO₃ (3.5 g, 32.6 mmol, 2.5 eq). The resulting solution was stirred overnight at 100° C. The resulting mixture was quenched by the addition of water (200 mL) and extracted with ethyl acetate (3×200 mL). The combined organic layer was washed with brine (200 mL), dried over anhydrous Na₂SO₄. After filtration, the filtrate was concentrated under vacuum. The crude residue was applied onto a silica gel column and eluted with ethyl acetate/petroleum ether=1:10 to give methyl 2-bromo-4-{1-methyl-2-oxo-7-azaspiro[3.5]nonan-7-yl}benzoate as alight yellow oil (3.4 g, 71.1%). ¹H NMR (300 MHz, Chloroform-d) δ 7.84 (d, J=8.9 Hz, 1H), 7.16 (d, J=2.6 Hz, 1H), 6.85 (dd, J=8.9, 2.6 Hz, 1H), 3.89 (s, 3H), 3.81-3.67 (m, 2H), 3.08 (dd, J=5.7, 4.0 Hz, 1H), 3.08-2.96 (m, 2H), 2.90 (ddd, J=16.8, 2.8, 1.3 Hz, 1H), 2.80 (dd, J=16.9, 1.7 Hz, 1H), 2.14-1.99 (m, 1H), 1.91-1.74 (m, 2H), 1.56 (ddd, J=14.1, 8.0, 3.9 Hz, 1H), 1.10 (d, J=7.5 Hz, 3H).

Synthesis of 2-bromo-4-{1-methyl-2-oxo-7-azaspiro[3.5]nonan-7-yl}benzoic acid: Into a 100 mL round-bottom flask, were placed methyl 2-bromo-4-{1-methyl-2-oxo-7-azaspiro[3.5]nonan-7-yl}benzoate (2.5 g, 6.8 mmol, 1.0 eq), methanol (12.5 mL), 1,4-dioxane (12.5 mL), NaOH (1.1 g, 27.3 mmol, 4.0 eq). The reaction mixture was stirred for 16 hours at 45° C. The resulting mixture was then diluted with water (30 mL) and acidified to pH=5 with HCl (1 M). The precipitated solids were collected by filtration and dried under infrared light to give 2-bromo-4-{1-methyl-2-oxo-7-azaspiro[3.5]nonan-7-yl}benzoic acid as a yellow solid (2.0 g, crude). LC-MS (ESI, m/z) M+1: 352.

Synthesis of 2-bromo-4-{1-methyl-2-oxo-7-azaspiro[3.5]nonan-7-yl}-N-[3-nitro-4-({[(1r,4r)-4-hydroxy-4-methylcyclohexyl]methyl}amino)benzenesulfonyl]benzamide: Into a 40 mL vial, were placed 2-bromo-4-{1-methyl-2-oxo-7-azaspiro[3.5]nonan-7-yl}benzoic acid (1.0 g, 2.9 mmol, 1.0 eq), 3-nitro-4-({[(1r,4r)-4-hydroxy-4-methylcyclohexyl]methyl}amino)benzenesulfonamide (970 mg, 2.8 mmol, 1.0 eq), dichloromethane (10 mL), EDCl (1.1 g, 5.7 mmol, 2.0 eq), DMAP (1.4 g, 11.4 mmol, 4.0 eq). The reaction mixture was stirred overnight at 25° C. The resulting mixture was concentrated under vacuum. The crude residue was applied onto a silica gel column and eluted with dichloromethane/ethyl acetate (included 20% methanol)=1:1 to give 2-bromo-4-{1-methyl-2-oxo-7-azaspiro[3.5]nonan-7-yl}-N-[3-nitro-4-({[(1r,4r)-4-hydroxy-4-methylcyclohexyl]methyl}amino)benzenesulfonyl]benzamide as a yellow solid (1.2 g, 62.4%). LC-MS (ESI, m/z) M+1: 677.

Synthesis of 4-{1-methyl-2-oxo-7-azaspiro[3.5]nonan-7-yl}-N-[3-nitro-4-({[(1r,4r)-4-hydroxy-4-methylcyclohexyl]methyl}amino)benzenesulfonyl]-2-[(3R,8S)-15-{[2-(trimethylsilyl)ethoxy]methyl}-2,5-dioxa-9,15,17-triazatetracyclo[8.7.0.0\hat{ }{3,8}.0\hat{ }{12,16}]heptadeca-1(10),11,13,16-tetraen-9-yl]benzamide: Into an 8-mL sealed tube purged and maintained with an inert atmosphere of nitrogen, were placed 2-bromo-4-{1-methyl-2-oxo-7-azaspiro[3.5]nonan-7-yl}-N-[3-nitro-4-({[(1r,4r)-4-hydroxy-4-methylcyclohexyl]methyl}amino)benzenesulfonyl]benzamide (800 mg, 1.2 mmol, 1.0 eq), (3R,8S)-15-{[2-(trimethylsilyl)ethoxy]methyl}-2,5-dioxa-9,15,17-triazatetracyclo[8.7.0.0\hat{ }{3,8}.0\hat{ }{12,16}]heptadeca-1(10),11,13,16-tetraene (427 mg, 1.2 mmol, 1.0 eq), N1,N2-di-o-tolyloxalamide (170 mg, 0.7 mmol, 0.6 eq), K₂CO₃ (490 mg, 3.5 mmol, 3.0 eq), Cul (135 mg, 0.7 mmol, 0.6 eq), DMSO (1 mL). The resulting solution was stirred for 2 hours at 100° C. The resulting mixture was then quenched by the addition of water (30 mL), and extracted with ethyl acetate (2×30 mL). The combined organic layers was washed with brine (2×30 mL) and dried over anhydrous Na₂SO₄. After filtration, the filtrate was concentrated under vacuum. The crude residue was applied onto a silica gel column and eluted with dichloromethane/methanol=10:1 to give 4-{1-methyl-2-oxo-7-azaspiro[3.5]nonan-7-yl}-N-[3-nitro-4-({[(1r,4r)-4-hydroxy-4-methylcyclohexyl]methyl}amino)benzenesulfonyl]-2-[(3R,8S)-15-{[2-(trimethylsilyl)ethoxy]methyl}-2,5-dioxa-9,15,17-triazatetracyclo[8.7.0.0\hat{ }{3,8}.0\hat{ }{12,16}]heptadeca-1(10),11,13,16-tetraen-9-yl]benzamide as a yellow solid (300 mg, 26.5%). LC-MS (ESI, m/z) M+1: 958.

### Example INT_63 Preparation of 4-(2-((R)-4-((5-methoxy-6-morpholinopyridin-3-yl)methyl)-2-(o-tolyl)piperazin-1-yl)-7-azaspiro[3.5]nonan-7-yl)-2-((5aS,9aR)-1-((2-(trimethylsilyl)ethoxy)methyl)-6,7,9,9a-tetrahydro-1H-pyrano[3,4-b]pyrrolo[3′,2′:5,6]pyrido[3,2-e][1,4]oxazin-5(5aH)-yl)benzoic acid

Synthesis of methyl 4-(2-((R)-4-((5-methoxy-6-morpholinopyridin-3-yl)methyl)-2-(o-tolyl)piperazin-1-yl)-7-azaspiro[3.5]nonan-7-yl)-2-((5aS,9aR)-1-((2-(trimethylsilyl)ethoxy)methyl)-6,7,9,9a-tetrahydro-1H-pyrano[3,4-b]pyrrolo[3′,2′:5,6]pyrido[3,2-e][1,4]oxazin-5(5aH)-yl)benzoate: Into a 50 mL round-bottom flask were added (R)-4-(3-methoxy-5-((3-(o-tolyl)piperazin-1-yl)methyl)pyridin-2-yl)morpholine (400 mg, 0.9 mmol, 1.0 eq), methyl 4-{2-oxo-7-azaspiro[3.5]nonan-7-yl}-2-[(3R,8S)-15-{[2-(trimethylsilyl)ethoxy]methyl}-2,5-dioxa-9,15,17-triazatetracyclo[8.7.0.0\hat{ }{3,8}.0\hat{ }{12,16}]heptadeca-1(10),11,13,16-tetraen-9-yl]benzoate (593 mg, 0.9 mmol, 1.0 eq), NaBH₃CN (294 mg, 4.7 mmol, 5.0 eq), ZnCl₂ (639 mg, 4.7 mmol, 5.0 eq) and MeOH (8 mL). The resulting mixture was stirred for 4 hours at 50° C. The reaction was quenched by the addition of water (10 mL). The resulting mixture was extracted with CH₂Cl₂ (3×10 mL). The combined organic layers were washed with brine (3×10 mL), dried over anhydrous Na₂SO₄. After filtration, the filtrate was concentrated under vacuum. The crude residue was applied onto a silica gel column and eluted with ethyl acetate/petroleum ether=1:1 to give methyl 4-(2-((R)-4-((5-methoxy-6-morpholinopyridin-3-yl)methyl)-2-(o-tolyl)piperazin-1-yl)-7-azaspiro[3.5]nonan-7-yl)-2-((5aS,9aR)-1-((2-(trimethylsilyl)ethoxy)methyl)-6,7,9,9a-tetrahydro-1H-pyrano[3,4-b]pyrrolo[3′,2′:5,6]pyrido[3,2-e][1,4]oxazin-5(5aH)-yl)benzoate as a greenish solid (560 mg, 57.2%). LC-MS (ESI, m/z) M+1: 1000.

Synthesis of 4-(2-((R)-4-((5-methoxy-6-morpholinopyridin-3-yl)methyl)-2-(o-tolyl)piperazin-1-yl)-7-azaspiro[3.5]nonan-7-yl)-2-((5aS,9aR)-1-((2-(trimethylsilyl)ethoxy)methyl)-6,7,9,9a-tetrahydro-1H-pyrano[3,4-b]pyrrolo[3′,2′:5,6]pyrido[3,2-e][1,4]oxazin-5(5aH)-yl)benzoic acid: Into a 50 mL round-bottom flask were added methyl 4-(2-((R)-4-((5-methoxy-6-morpholinopyridin-3-yl)methyl)-2-(o-tolyl)piperazin-1-yl)-7-azaspiro[3.5]nonan-7-yl)-2-((5aS,9aR)-1-((2-(trimethylsilyl)ethoxy)methyl)-6,7,9,9a-tetrahydro-1H-pyrano[3,4-b]pyrrolo[3′,2′:5,6]pyrido[3,2-e][1,4]oxazin-5(5aH)-yl)benzoate (500 mg, 0.5 mmol, 1.0 eq), NaOH(4 M, 2 mL) and dioxane/MeOH (5 mL/5 mL). The resulting mixture was stirred for overnight at 50° C. The resulting mixture was concentrated under vacuum. The resulting mixture was diluted with water (10 mL), and extracted with CH₂Cl₂ (3×10 mL). The combined organic layers were washed with brine (10 mL), dried over anhydrous Na₂SO₄. After filtration, the filtrate was concentrated under vacuum to give 4-(2-((R)-4-((5-methoxy-6-morpholinopyridin-3-yl)methyl)-2-(o-tolyl)piperazin-1-yl)-7-azaspiro[3.5]nonan-7-yl)-2-((5aS,9aR)-1-((2-(trimethylsilyl)ethoxy)methyl)-6,7,9,9a-tetrahydro-1H-pyrano[3,4-b]pyrrolo[3′,2′:5,6]pyrido[3,2-e][1,4]oxazin-5(5aH)-yl)benzoic acid as a yellow green solid (410 mg, 83.1%). LC-MS (ESI, m/z) M+1: 986.

### Example INT_64 Preparation of 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-N-[(3R)-5-nitro-3-(oxan-4-yl)-3,4-dihydro-2H-1,4-benzoxazin-7-ylsulfonyl]-4-{2-oxo-7-azaspiro[3.5]nonan-7-yl}benzamide

Synthesis of 2-bromo-N-[(3R)-5-nitro-3-(oxan-4-yl)-3,4-dihydro-2H-1,4-benzoxazin-7-ylsulfonyl]-4-{2-oxo-7-azaspiro[3.5]nonan-7-yl}benzamide: Into a 100 mL round-bottom flask were added 2-bromo-4-{2-oxo-7-azaspiro[3.5]nonan-7-yl}benzoic acid (1.1 g, 3.2 mmol, 1.0 eq), (3R)-5-nitro-3-(oxan-4-yl)-3,4-dihydro-2H-1,4-benzoxazine-7-sulfonamide (1.1 g, 3.2 mmol, 1.0 eq), EDCl (1.2 g, 6.5 mmol, 2.0 eq), DMAP (0.4 g, 3.2 mmol, 1.0 eq) and DCM (10 mL). The resulting mixture was stirred for 2 hours at 25° C. The resulting mixture was diluted with CH₂Cl₂ (20 mL) and washed with water (3×10 mL), dried over anhydrous Na₂SO₄. After filtration, the filtrate was concentrated under vacuum. The crude residue was purified onto a silica gel column and eluted with CH₂Cl₂/MeOH=30:1 to give 2-bromo-N-[(3R)-5-nitro-3-(oxan-4-yl)-3,4-dihydro-2H-1,4-benzoxazin-7-ylsulfonyl]-4-{2-oxo-7-azaspiro[3.5]nonan-7-yl}benzamide as an orange solid (1.0 g, 46.3%). LC-MS (ESI, m/z) M+1: 663/665.

Synthesis of N-[(3R)-5-nitro-3-(oxan-4-yl)-3,4-dihydro-2H-1,4-benzoxazin-7-ylsulfonyl]-4-{2-oxo-7-azaspiro[3.5]nonan-7-yl}-2-[(3R,8S)-15-{[2-(trimethylsilyl)ethoxy]methyl}-2,5-dioxa-9,15,17-triazatetracyclo[8.7.0.0\hat{ }{3,8}.0\hat{ }{12,16}]heptadeca-1(10),11,13,16-tetraen-9-yl]benzamide: Into a 100 mL round-bottom flask were added 2-bromo-N-[(3R)-5-nitro-3-(oxan-4-yl)-3,4-dihydro-2H-1,4-benzoxazin-7-ylsulfonyl]-4-{2-oxo-7-azaspiro[3.5]nonan-7-yl}benzamide (1.0 g, 1.5 mmol, 1.0 eq), (3R,8S)-15-{[2-(trimethylsilyl)ethoxy]methyl}-2,5-dioxa-9,15,17-triazatetracyclo[8.7.0.0\hat{ }{3,8}.0\hat{ }{12,16}]heptadeca-1(10),11,13,16-tetraene (0.5 g, 1.5 mmol, 1.0 eq), ethanediamide, N,N′-diphenyl-(0.2 g, 0.9 mmol, 0.6 eq), Cul (0.1 g, 0.6 mmol, 0.4 eq), K₂CO₃ (0.6 g, 4.5 mmol, 3.0 eq) and DMSO (20 mL). The resulting mixture was stirred for 2 hours at 100° C. under nitrogen atmosphere. The precipitated solids were collected by filtration and washed with CH₂Cl₂ (3×10 mL). The resulting mixture was diluted with water (20 mL). The resulting mixture was extracted with CH₂Cl₂ (3×20 mL). The combined organic layers were washed with brine (20 mL), dried over anhydrous Na₂SO₄. After filtration, the filtrate was concentrated under vacuum. The crude residue was purified by silica gel column chromatography, eluted with CH₂Cl₂/MeOH (15:1) to give N-[(3R)-5-nitro-3-(oxan-4-yl)-3,4-dihydro-2H-1,4-benzoxazin-7-ylsulfonyl]-4-{2-oxo-7-azaspiro[3.5]nonan-7-yl}-2-[(3R,8S)-15-{[2-(trimethylsilyl)ethoxy]methyl}-2,5-dioxa-9,15,17-triazatetracyclo[8.7.0.0\hat{ }{3,8}.0\hat{ }{12,16}]heptadeca-1(10),11,13,16-tetraen-9-yl]benzamide as an orange solid (600 mg, 42.2%). LC-MS (ESI, m/z) M+1: 944.

Synthesis of 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-N-[(3R)-5-nitro-3-(oxan-4-yl)-3,4-dihydro-2H-1,4-benzoxazin-7-ylsulfonyl]-4-{2-oxo-7-azaspiro[3.5]nonan-7-yl}benzamide: Into a 50 mL round-bottom flask were added N-[(3R)-5-nitro-3-(oxan-4-yl)-3,4-dihydro-2H-1,4-benzoxazin-7-ylsulfonyl]-4-{2-oxo-7-azaspiro[3.5]nonan-7-yl}-2-[(3R,8S)-15-{[2-(trimethylsilyl)ethoxy]methyl}-2,5-dioxa-9,15,17-triazatetracyclo[8.7.0.0\hat{ }{3,8}.0\hat{ }{12,16}]heptadeca-1(10),11,13,16-tetraen-9-yl]benzamide (600 mg, 0.6 mmol, 1.0 eq) and TFA (30 mL) at 0° C. The resulting mixture was stirred for 2 hours at 25° C. The resulting mixture was concentrated under vacuum. The residue was dissolved in DCM (10 mL). The resulting mixture was concentrated under vacuum. To the above mixture was added butylamine (45 mL) at 0° C. The resulting mixture was stirred for 15 hours at 25° C. The resulting mixture was concentrated under vacuum. The crude residue was dissolved in DCM (10 mL). The resulting mixture was concentrated under vacuum. The crude residue was purified onto a silica gel column and eluted with CH₂Cl₂/MeOH=13:1 to give 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-N-[(3R)-5-nitro-3-(oxan-4-yl)-3,4-dihydro-2H-1,4-benzoxazin-7-ylsulfonyl]-4-{2-oxo-7-azaspiro[3.5]nonan-7-yl}benzamide as a yellow solid (240 mg, 46.4%). LC-MS (ESI, m/z) M+1: 814.

### Example INT_65 Preparation of 8-chloro-4,4-dimethyl-2H,3H-pyrano[2,3-c]pyridine

Synthesis of 2-chloro-3-[(3-methylbut-3-en-1-yl)oxy]pyridine: Into a 500 mL 3-necked round-bottom flask, were placed 2-chloro-3-pyridinol, (25.0 g, 193.0 mmol, 1.0 eq), 3-methyl-3-buten-1-ol (16.6 g, 193.0 mmol, 1.0 eq), THE (250 mL), PPh₃ (55.7 g, 212.3 mmol, 1.1 eq). This was followed by the addition of DIAD (42.9 g, 212.3 mmol, 1.1 eq) at 0° C. The resulting mixture was stirred for 2 hours at 25° C. The resulting mixture was then quenched by the addition of water (500 mL), and then extracted with ethyl acetate (2×500 mL). The combined organic layers was washed with brine (2×500 mL), and dried over anhydrous Na₂SO₄. After filtration, the filtrate was concentrated under vacuum. The crude residue was purified onto a silica gel column and eluted with ethyl acetate/petroleum ether=1:2 to give 2-chloro-3-[(3-methylbut-3-en-1-yl)oxy]pyridine as a colorless oil (31.0 g, 81.3%). ¹H NMR (300 MHz, DMSO-d₆) δ 7.96 (dd, J=4.8, 1.5 Hz, 1H), 7.60 (dd, J=8.1, 1.5 Hz, 1H), 7.37 (dd, J=8.1, 4.6 Hz, 1H), 4.86-4.77 (m, 2H), 4.20 (t, J=6.6 Hz, 2H), 2.47 (t, J=6.6 Hz, 2H), 1.78 (t, J=1.2 Hz, 3H).

Synthesis of 8-chloro-4,4-dimethyl-2H,3H-pyrano[2,3-c]pyridine: Into a 1 L 3-necked round-bottom flask, were placed 2-chloro-3-[(3-methylbut-3-en-1-yl)oxy]pyridine (30.0 g, 151.8 mmol, 1.0 eq), TFA (34.6 g, 303.6 mmol, 2.0 eq), EtOH (300 mL), Fe(acac)₃ (26.8 g, 75.9 mmol, 0.5 eq), phenylsilane (41.1 g, 379.4 mmol, 2.5 eq), t-butyl-peroxide (66.6 g, 455.3 mmol, 3.0 eq). The resulting mixture was stirred for 16 hours at 60° C. The reaction mixture was quenched by the addition of water (500 mL), extracted with ethyl acetate (2×500 mL). The combined organic layers was washed with brine (2×500 mL), dried over anhydrous Na₂SO₄. After filtration, the filtrate was concentrated under vacuum. The crude residue was purified onto a silica gel column and eluted with ethyl acetate/petroleum ether=1:5 to give 8-chloro-4,4-dimethyl-2H,3H-pyrano[2,3-c]pyridine as a light yellow oil (15.0 g, 50.0%). ¹H NMR (300 MHz, DMSO-d₆) δ 7.85 (d, J=5.1 Hz, 1H), 7.38 (d, J=5.1 Hz, 1H), 4.38-4.23 (m, 2H), 1.83 (dd, J=6.3, 4.5 Hz, 2H), 1.30 (s, 6H).

### Example INT_66 Preparation of (3R)-1-[(1R)-1-(3,4-difluorophenyl)ethyl]-3-(2-isopropoxyphenyl)piperazine (assumed) & (3R)-1-[(1S)-1-(3,4-difluorophenyl)ethyl]-3-(2-isopropoxyphenyl)piperazine (assumed)

Synthesis of 1-(3,4-difluorophenyl)ethanol: Into a 100 mL 3-necked round-bottom, were placed 1-(3,4-difluorophenyl)ethanone (3.0 g, 19.2 mmol, 1.0 eq), MeOH (30 mL) at 0° C. To the above mixture was added NaBH₄ (1.4 g, 38.4 mmol, 2.0 eq) at 0° C. The resulting mixture was stirred for additional 2 hours at 25° C. The resulting mixture was then quenched by the addition of water (50 mL), and then extracted with ethyl acetate (2×50 mL). The combined organic layers was washed with brine (2×50 mL), dried over anhydrous Na₂SO₄. After filtration, the filtrate was concentrated under vacuum. The crude residue was purified onto a silica gel column and eluted with ethyl acetate/petroleum ether=1:3 to give 1-(3,4-difluorophenyl)ethanol as a colorless oil (2.5 g, 82.3%). ¹H NMR (300 MHz, DMSO-d₆) δ 7.43-7.28 (m, 2H), 7.22-7.13 (m, 1H), 5.33 (d, J=4.5 Hz, 1H), 4.72 (dd, J=6.6, 4.5 Hz, 1H), 1.31 (d, J=6.6 Hz, 3H).

Synthesis of 4-(1-chloroethyl)-1,2-difluorobenzene: Into a 40 mL sealed tube, were placed 1-(3,4-difluorophenyl)ethanol (500 mg, 3.2 mmol, 1.0 eq), CHCl₃ (2 mL) at 0° C. To the above mixture was added SOCl₂ (3 mL) at 0° C. The resulting mixture was stirred for additional 4 hours at 25° C. The resulting mixture was then quenched by the addition of water (50 mL), and then extracted with dichloromethane (2×50 mL). The combined organic layers was washed with brine (2×50 mL), dried over anhydrous Na₂SO₄. After filtration, the filtrate was concentrated under vacuum. The crude residue was purified onto a silica gel column and eluted with ethyl acetate/petroleum ether=1:3 to give 4-(1-chloroethyl)-1,2-difluorobenzene as a light yellow oil (300 mg, 53.7%). ¹H NMR (300 MHz, DMSO-d₆) δ 7.60 (ddd, J=11.7, 7.8, 2.1 Hz, 1H), 7.50-7.31 (m, 2H), 5.36 (q, J=6.9 Hz, 1H), 1.78 (d, J=6.9 Hz, 3H).

Synthesis of (3R)-1-[(1R)-1-(3,4-difluorophenyl)ethyl]-3-(2-isopropoxyphenyl)piperazine (assumed) & (3R)-1-[(1S)-1-(3,4-difluorophenyl)ethyl]-3-(2-isopropoxyphenyl)piperazine (assumed): Into an 8-mL sealed tube, were placed 4-(1-chloroethyl)-1,2-difluorobenzene (289 mg, 1.6 mmol, 1.2 eq), (2R)-2-(2-isopropoxyphenyl)piperazine (300 mg, 1.4 mmol, 1.0 eq), K₂CO₃ (565 mg, 4.1 mmol, 3.0 eq), CH₃CN (3 mL). The resulting solution was stirred for overnight at 80° C. The resulting mixture was then quenched by the addition of water (30 mL), and then extracted with dichloromethane (2×30 mL). The combined organic layers was washed with brine (2×30 mL), dried over anhydrous Na₂SO₄. After filtration, the filtrate was concentrated under vacuum. The crude residue was applied onto a silica gel column and eluted with ethyl acetate/petroleum ether=1:0 to give (3R)-1-[(1R)-1-(3,4-difluorophenyl)ethyl]-3-(2-isopropoxyphenyl)piperazine (assumed) as a light yellow oil (80 mg, 16.3%) and (3R)-1-[(1S)-1-(3,4-difluorophenyl)ethyl]-3-(2-isopropoxyphenyl)piperazine (assumed) as a light yellow oil (50 mg, 10.2%). ¹H NMR_A (300 MHz, Chloroform-d) δ 7.47 (dd, J=7.5, 1.8 Hz, 1H), 7.30-7.16 (m, 2H), 7.09 (dd, J=9.3, 6.6 Hz, 2H), 6.98-6.83 (m, 2H), 4.67-4.50 (m, 1H), 4.25 (dd, J=9.9, 2.7 Hz, 1H), 3.45 (q, J=6.6 Hz, 1H), 3.10-3.03 (m, 3H), 2.76-2.63 (m, 1H), 2.28-2.20 (m, 1H), 2.01 (t, J=10.2 Hz, 1H), 1.40-1.28 (m, 9H). ¹H NMR_B (300 MHz, Chloroform-d) δ 7.43 (dd, J=7.5, 1.8 Hz, 1H), 7.27-7.12 (m, 2H), 7.11-6.96 (m, 2H), 6.88 (t, J=7.5 Hz, 1H), 6.80 (d, J=8.1 Hz, 1H), 4.56-4.50 (m, 1H), 4.18 (dd, J=9.9, 2.7 Hz, 1H), 3.32 (q, J=6.6 Hz, 1H), 3.24-3.01 (m, 3H), 3.00-2.86 (m, 1H), 2.28-2.20 (m, 1H), 1.89-1.77 (m, 2H), 1.33 (dd, J=10.5, 6.3 Hz, 6H), 1.14 (d, J=6.0 Hz, 3H).

### Example INT_67 Preparation of 4-chloro-7,8-difluoro-3,4-dihydro-2H-1-benzopyran

Synthesis of 3-(2,3-difluorophenoxy)propanoic acid: Into a 250 mL round-bottom flask were added 2,3-difluorophenol (10.0 g, 76.9 mmol, 1.0 eq), 3-bromo-propanoic acid (13.0 g, 85.3 mmol, 1.1 eq), NaOH (6.2 g, 155.3 mmol, 2.0 eq) and water (100 mL). The resulting mixture was stirred for overnight at 100° C. The mixture was acidified to pH=2 with HCl (3 M). The resulting mixture was diluted with water (30 mL) and extracted with EtOAc (3×50 mL). The combined organic layers were washed with brine (50 mL), dried over anhydrous Na₂SO₄. After filtration, the filtrate was concentrated under vacuum. The residue was applied onto a silica gel column and eluted with ethyl acetate/petroleum ether=1:3 to give 3-(2,3-difluorophenoxy)propanoic acid as a colorless solid (4.1 g, 26.3%). ¹H NMR (300 MHz, Chloroform-d) δ 8.87 (s, 1H), 7.12-6.93 (m, 1H), 6.90-6.69 (m, 2H), 4.36 (t, J=6.3 Hz, 2H), 2.93 (t, J=6.3 Hz, 2H).

Synthesis of 7,8-difluoro-2,3-dihydro-1-benzopyran-4-one: Into a 100 mL round-bottom flask were added 3-(2,3-difluorophenoxy)propanoic acid (3.5 g, 17.3 mmol, 1.0 eq) and trifluoromethanesulfonic acid (35 mL) at 0° C. The resulting mixture was stirred for 18 hours at 25° C. The reaction was quenched by the addition of water/ice (50 mL) at 0° C. The precipitated solids were collected by filtration and washed with water (3×10 mL). Finally, 7,8-difluoro-2,3-dihydro-1-benzopyran-4-one was obtained as an off-white solid (3.0 g, 94.1%). ¹H NMR (300 MHz, Chloroform-d) δ 7.71 (ddd, J=9.0, 5.9, 2.3 Hz, 1H), 6.86 (td, J=9.2, 6.5 Hz, 1H), 4.69 (dd, J=6.8, 6.0 Hz, 2H), 2.88 (dd, J=6.8, 6.0 Hz, 2H).

Synthesis of 7,8-difluoro-3,4-dihydro-2H-1-benzopyran-4-ol: To a solution of 7,8-difluoro-2,3-dihydro-1-benzopyran-4-one (3.0 g, 19.7 mmol, 1.0 eq) in MeOH (10 mL) was added NaBH₄ (1.2 g, 32.6 mmol, 2.0 eq) in portions at 0° C. The resulting mixture was stirred for 2 hours at 25° C. The reaction was quenched by the addition of water (5 mL) at 0° C. and extracted with EtOAc (3×10 mL). The combined organic layers were washed with brine (10 mL), dried over anhydrous Na₂SO₄. After filtration, the filtrate was concentrated under vacuum to give 7,8-difluoro-3,4-dihydro-2H-1-benzopyran-4-ol as a white solid (1.3 g, 85.7%). ¹H NMR (300 MHz, Chloroform-d) δ 7.05 (ddd, J=8.3, 5.6, 2.3 Hz, 1H), 6.74 (ddd, J=9.7, 8.8, 7.0 Hz, 1H), 4.81 (t, J=4.1 Hz, 1H), 4.51-4.30 (m, 2H), 2.30-2.00 (m, 2H), 1.83 (s, 1H).

Synthesis of 4-chloro-7,8-difluoro-3,4-dihydro-2H-1-benzopyran: To a solution of 7,8-difluoro-3,4-dihydro-2H-1-benzopyran-4-ol (750 mg, 4.0 mmol, 1.0 eq) in CHCl₃ (5 mL) was added SOCl₂ (5 mL, 68.9 mmol, 17.1 eq) dropwise at 0° C. The resulting mixture was stirred for 4 hours at 25° C. The resulting mixture was concentrated under vacuum. The resulting mixture was diluted with water (10 mL) and extracted with EtOAc (3×10 mL). The combined organic layers were washed with brine (10 mL), dried over anhydrous Na₂SO₄. After filtration, the filtrate was concentrated under vacuum to give 4-chloro-7,8-difluoro-3,4-dihydro-2H-1-benzopyran as an orange oil (620 mg, 72.2%). ¹H NMR (300 MHz, Chloroform-d) δ 7.04 (dddd, J=8.7, 5.6, 2.4, 0.6 Hz, 1H), 6.74 (ddd, J=9.6, 8.8, 7.0 Hz, 1H), 5.27-5.19 (m, 1H), 4.64-4.43 (m, 2H), 2.64-2.25 (m, 2H).

### Example INT_68 Preparation of 4-chloro-6,8-difluorochromane

Synthesis of 3-(2,4-difluorophenoxy)propanoic acid: Into a 250 mL round-bottom flask were added 2,4-difluorophenol (10.0 g, 76.9 mmol, 1.0 eq), 3-bromo-propanoic acid (13.0 g, 85.3 mmol, 1.1 eq), NaOH (6.2 g, 155.3 mmol, 2.0 eq) and water (100 mL). The resulting mixture was stirred for overnight at 100° C. The mixture was acidified to pH=2 with HCl (3 M). The resulting mixture was diluted with water (30 mL) and extracted with EtOAc (3×50 mL). The combined organic layers were washed with brine (50 mL), dried over anhydrous Na₂SO₄. After filtration, the filtrate was concentrated under vacuum. The residue was applied onto a silica gel column and eluted with ethyl acetate/petroleum ether=1:3 to give 3-(2,4-difluorophenoxy)propanoic acid as a colorless solid (4.1 g, 26.3%). ¹H NMR (400 MHz, DMSO-d₆) δ 12.40 (s, 1H), 7.41-7.15 (m, 2H), 7.01-7.00 (m, 1H), 4.22 (t, J=6.0 Hz, 2H), 2.71 (t, J=6.0 Hz, 2H).

Synthesis of 6,8-difluorochroman-4-one: Into a 100 mL round-bottom flask were added 3-(2,4-difluorophenoxy)propanoic acid (3.5 g, 17.3 mmol, 1.0 eq) and trifluoromethanesulfonic acid (35 mL) at 0° C. The resulting mixture was stirred for 18 hours at 25° C. The reaction was quenched by the addition of water/ice (50 mL) at 0° C. The precipitated solids were collected by filtration and washed with water (3×10 mL). Finally, 6,8-difluorochroman-4-one was obtained as an off-white solid (2.2 g, 84.1%). ¹H NMR (400 MHz, DMSO-d₆) δ 7.69-7.68 (m, 1H), 7.33-7.30 (m, 1H), 4.65 (t, J=6.5 Hz, 2H), 2.89 (t, J=6.5 Hz, 2H).

Synthesis of 6,8-difluorochroman-4-ol: To a solution of 6,8-difluorochroman-4-one (1.0 g, 19.7 mmol, 1.0 eq) in MeOH (10 mL) was added NaBH₄ (1.2 g, 32.6 mmol, 2.0 eq) in portions at 0° C. The resulting mixture was stirred for 2 hours at 25° C. The reaction was quenched by the addition of water (5 mL) at 0° C. and extracted with EtOAc (3×10 mL). The combined organic layers were washed with brine (10 mL), dried over anhydrous Na₂SO₄. After filtration, the filtrate was concentrated under vacuum to give 6,8-difluorochroman-4-ol as a white solid (0.9 g, 85.7%). ¹H NMR (300 MHz, Chloroform-d) δ 6.89-6.77 (m, 2H), 5.17 (t, J=3.7 Hz, 1H), 4.55-4.34 (m, 2H), 2.61-2.46 (m, 1H), 2.35-2.33 (m, 1H).

Synthesis of 4-chloro-6,8-difluorochromane: To a solution of 6,8-difluorochroman-4-ol (700 mg, 4.0 mmol, 1.0 eq) in CHCl₃ (5 mL) was added SOCl₂ (5 mL, 68.9 mmol, 17.1 eq) dropwise at 0° C. The resulting mixture was stirred for 4 hours at 25° C. The resulting mixture was concentrated under vacuum. The resulting mixture was diluted with water (10 mL) and extracted with EtOAc (3×10 mL). The combined organic layers were washed with brine (10 mL), dried over anhydrous Na₂SO₄. After filtration, the filtrate was concentrated under vacuum to give 4-chloro-6,8-difluorochromane as an orange solid (500 mg, 72.2%). ¹H NMR (300 MHz, Chloroform-d) δ 6.86 (d, J=3.0 Hz, 1H), 6.81-6.75 (m, 1H), 5.17 (t, J=3.7 Hz, 1H), 4.49-4.45 (m, 3H), 2.53-2.50 (m, 1H).

### Example INT_69 Preparation of 3-oxaspiro[5.5]undecan-9-one

Synthesis of 3-oxaspiro[5.5]undec-7-en-9-one: Into a 100 mL round-bottom flask, were added oxane-4-carbaldehyde (5.0 g, 43.8 mmol, 1.0 eq), toluene (70 mL), methyl vinyl ketone (3.6 mL, 48.1 mmol, 1.1 eq), H₂SO₄ (0.1 mL, 1.3 mmol, 0.03 equiv) was heating with a water separator at 110° C. After 3 hours another batch of methyl vinyl ketone (3.56 mL) was added and the mixture was refluxed for overnight. The reaction was quenched with water (50 mL) and extracted with ethyl acetate (3×70 mL). The combined organic layers were dried over anhydrous Na₂SO₄. After filtration, the filtrate was concentrated under vacuum. The crude residue was purified onto a silica gel column and eluted with petroleum ether/ethyl acetate=3:1 to give 3-oxaspiro[5.5]undec-7-en-9-one as a brown oil (2.0 g, 27.4%). LC-MS (ES, m/z) M+1: 167.

Synthesis of 3-oxaspiro[5.5]undecan-9-one: To a solution of 3-oxaspiro[5.5]undec-7-en-9-one (500 mg, 3.0 mmol, 1.0 eq) in 20 mL MeOH was added Pd/C (10%, 50 mg) under nitrogen atmosphere in a 100 mL round-bottom flask. The mixture was hydrogenated at 25° C. for 3 hours under hydrogen atmosphere using a hydrogen balloon, after filtered through a Celite pad and concentrated under vacuum. Finally, 3-oxaspiro[5.5]undecan-9-one was obtained as a brown oil (300 mg, 59.2%). LC-MS (ES, m/z) M+1: 169.

### Example INT_70 Preparation of 4-chloro-6,7-difluoro-3,4-dihydro-2H-1-benzopyran

Synthesis of 3-(3,4-difluorophenoxy)propanoic acid: Into a 250 mL round-bottom flask were added 3,4-difluorophenol (10.0 g, 76.9 mmol, 1.0 eq), 3-bromo-propanoic acid (13.0 g, 85.3 mmol, 1.1 eq), NaOH (6.2 g, 155.3 mmol, 2.0 eq) and water (100 mL). The resulting mixture was stirred for overnight at 100° C. The mixture was acidified to pH=2 with HCl (3 M). The resulting mixture was diluted with water (30 mL), and then extracted with EtOAc (3×50 mL). The combined organic layers were washed with brine (50 mL), dried over anhydrous Na₂SO₄. After filtration, the filtrate was concentrated under vacuum. The residue was applied onto a silica gel column and eluted with ethyl acetate/petroleum ether=1:3 to give 3-(3,4-difluorophenoxy)propanoic acid as a colorless solid (4.1 g, 26.3%). ¹H NMR (300 MHz, Chloroform-d) δ 9.46 (s, 1H), 7.08 (dt, J=10.1, 9.1 Hz, 1H), 6.76 (ddd, J=11.9, 6.6, 3.0 Hz, 1H), 6.63 (dtd, J=9.1, 3.2, 1.7 Hz, 1H), 4.22 (t, J=6.2 Hz, 2H), 2.87 (t, J=6.2 Hz, 2H).

Synthesis of 6,7-difluoro-2,3-dihydro-1-benzopyran-4-one: Into a 100 mL round-bottom flask were added 3-(3,4-difluorophenoxy)propanoic acid (3.5 g, 17.3 mmol, 1.0 eq) and trifluoromethanesulfonic acid (30 mL) at 0° C. The resulting mixture was stirred for 18 hours at 25° C. The reaction was quenched by the addition of water/ice (50 mL) at 0° C. The precipitated solids were collected by filtration and washed with water (3×10 mL). Finally, 6,7-difluoro-2,3-dihydro-1-benzopyran-4-one was obtained as an off-white solid (2.2 g, 69.0%). ¹H NMR (300 MHz, Chloroform-d) δ 7.70 (dd, J=10.0, 9.0 Hz, 1H), 6.81 (dd, J=10.8, 6.3 Hz, 1H), 4.65-4.50 (m, 2H), 2.94-2.68 (m, 2H).

Synthesis of 6,7-difluoro-3,4-dihydro-2H-1-benzopyran-4-ol: To a solution of 6,7-difluoro-2,3-dihydro-1-benzopyran-4-one (1.5 g, 8.1 mmol, 1.0 eq) in MeOH (10 mL) was added NaBH₄ (0.9 g, 24.4 mmol, 3.0 eq) in portions at 0° C. The resulting mixture was stirred for 2 hours at 25° C. The reaction was quenched by the addition of water (5 mL) at 0° C., and then extracted with EtOAc (3×10 mL). The combined organic layers were washed with brine (10 mL), dried over anhydrous Na₂SO₄. After filtration, the filtrate was concentrated under vacuum to give 6,7-difluoro-3,4-dihydro-2H-1-benzopyran-4-ol as a white solid (1.3 g, 85.7%). ¹H NMR (300 MHz, Chloroform-d) δ 7.14 (dd, J=10.4, 8.8 Hz, 1H), 6.66 (dd, J=11.5, 6.9 Hz, 1H), 4.75 (t, J=4.4 Hz, 1H), 4.34-4.19 (m, 2H), 2.20-1.99 (m, 2H), 1.81 (s, 1H).

Synthesis of 4-chloro-6,7-difluoro-3,4-dihydro-2H-1-benzopyran: To a solution of 6,7-difluoro-3,4-dihydro-2H-1-benzopyran-4-ol (750 mg, 4.0 mmol, 1.0 eq) in CHCl₃ (5 mL) was added SOCl₂ (5 mL, 68.9 mmol, 17.1 eq) dropwise at 0° C. The resulting mixture was stirred for 4 hours at 25° C. The resulting mixture was concentrated under vacuum. The resulting mixture was diluted with water (10 mL), and extracted with EtOAc (3×10 mL). The combined organic layers were washed with brine (10 mL), dried over anhydrous Na₂SO₄. After filtration, the filtrate was concentrated under vacuum to afford 4-chloro-6,7-difluoro-3,4-dihydro-2H-1-benzopyran as an orange oil (610 mg, 74.0%). ¹H NMR (300 MHz, Chloroform-d) δ 7.11 (dd, J=10.3, 8.6 Hz, 1H), 6.65 (dd, J=11.3, 6.9 Hz, 1H), 5.15 (t, J=3.7 Hz, 1H), 4.53-4.25 (m, 2H), 2.59-2.38 (m, 1H), 2.31 (dq, J=15.0, 3.0 Hz, 1H).

### Example 99 Preparation of 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-4-{2-[(2S)-2-(2-isopropoxyphenyl)-4-[(4-methoxyphenyl)methyl]piperazin-1-yl]-7-azaspiro[3.5]nonan-7-yl}-N-[3-nitro-4-({[(1r,4r)-4-hydroxy-4-methylcyclohexyl]methyl}amino)benzenesulfonyl]benzamide (assumed)

Synthesis of tert-butyl (3S)-3-(2-isopropoxyphenyl)-4-[7-(4-{[3-nitro-4-({[(1r,4r)-4-hydroxy-4-methylcyclohexyl]methyl}amino)benzenesulfonyl]carbamoyl}-3-[(3R,8S)-15-{[2-(trimethylsilyl)ethoxy]methyl}-2,5-dioxa-9,15,17-triazatetracyclo[8.7.0.0\hat{ }{3,8}.0\hat{ }{12,16}]heptadeca-1(10),11,13,16-tetraen-9-yl]phenyl)-7-azaspiro[3.5]nonan-2-yl]piperazine-1-carboxylate (assumed): Into a 40 mL vial were added N-[3-nitro-4-({[(1r,4r)-4-hydroxy-4-methylcyclohexyl]methyl}amino)benzenesulfonyl]-4-{2-oxo-7-azaspiro[3.5]nonan-7-yl}-2-[(3R,8S)-15-{[2-(trimethylsilyl)ethoxy]methyl}-2,5-dioxa-9,15,17-triazatetracyclo[8.7.0.0\hat{ }{3,8}.0\hat{ }{12,16}]heptadeca-1(10),11,13,16-tetraen-9-yl]benzamide (800 mg, 0.8 mmol, 1.0 eq), tert-butyl (3S)-3-(2-isopropoxyphenyl)piperazine-1-carboxylate (272 mg, 0.8 mmol, 1.0 eq), NaBH₃CN (266 mg, 4.2 mmol, 5.0 eq), ZnCl₂ (577 mg, 4.2 mmol, 5.0 eq) and MeOH (10 mL). The resulting mixture was stirred for 4 hours at 70° C. The reaction was quenched by the addition of water (5 mL) and extracted with CH₂Cl₂ (3×20 mL). The combined organic layer was washed with brine (20 mL), dried over anhydrous Na₂SO₄. After filtration, the filtrate was concentrated under vacuum. The crude residue was purified by silica gel column chromatography and eluted with CH₂Cl₂/MeOH=30:1 to give tert-butyl (3S)-3-(2-isopropoxyphenyl)-4-[7-(4-{[3-nitro-4-({[(1r,4r)-4-hydroxy-4-methylcyclohexyl]methyl}amino)benzenesulfonyl]carbamoyl}-3-[(3R,8S)-15-{[2-(trimethylsilyl)ethoxy]methyl}-2,5-dioxa-9,15,17-triazatetracyclo[8.7.0.0\hat{ }{3,8}.0\hat{ }{12,16}]heptadeca-1(10),11,13,16-tetraen-9-yl]phenyl)-7-azaspiro[3.5]nonan-2-yl]piperazine-1-carboxylate (assumed) as a yellow solid (800 mg, 75.6%). LC-MS (ESI, m/z) M+1: 1249.

Synthesis of 2-[(3R,8S)-15-(hydroxymethyl)-2,5-dioxa-9,15,17-triazatetracyclo[8.7.0.0\hat{ }{3,8}.0\hat{ }{12,16}]heptadeca-1(10),11,13,16-tetraen-9-yl]-4-{2-[(2S)-2-(2-isopropoxyphenyl)piperazin-1-yl]-7-azaspiro[3.5]nonan-7-yl}-N-[3-nitro-4-({[(1r,4r)-4-hydroxy-4-methylcyclohexyl]methyl}amino)benzenesulfonyl]benzamide (assumed): Into a 50 mL round-bottom flask, were added tert-butyl (3S)-3-(2-isopropoxyphenyl)-4-[7-(4-{[3-nitro-4-({[(1r,4r)-4-hydroxy-4-methylcyclohexyl]methyl}amino)benzenesulfonyl]carbamoyl}-3-[(3R,8S)-15-{[2-(trimethylsilyl)ethoxy]methyl}-2,5-dioxa-9,15,17-triazatetracyclo[8.7.0.0\hat{ }{3,8}.0\hat{ }{12,16}]heptadeca-1(10),11,13,16-tetraen-9-yl]phenyl)-7-azaspiro[3.5]nonan-2-yl]piperazine-1-carboxylate (600 mg, 0.5 mmol, 1.0 eq) and TFA/DCM(3 mL/3 mL) at 0° C. The resulting mixture was stirred for 2 hours at 0° C. The mixture was basified to pH=8 with sat. NaHCO₃ and then extracted with CH₂Cl₂ (3×10 mL). The combined organic layer was washed with brine (10 mL), dried over anhydrous Na₂SO₄. After filtration, the filtrate was concentrated under vacuum. The crude residue was purified by Prep-TLC (CH₂Cl₂/MeOH=8:1) to give 2-[(3R,8S)-15-(hydroxymethyl)-2,5-dioxa-9,15,17-triazatetracyclo[8.7.0.0\hat{ }{3,8}.0\hat{ }{12,16}]heptadeca-1(10),11,13,16-tetraen-9-yl]-4-{2-[(2S)-2-(2-isopropoxyphenyl)piperazin-1-yl]-7-azaspiro[3.5]nonan-7-yl}-N-[3-nitro-4-({[(1r,4r)-4-hydroxy-4-methylcyclohexyl]methyl}amino)benzenesulfonyl]benzamide (assumed) as a yellow solid (280 mg, 55.6%). LC-MS (ESI, m/z) M+1: 1048.

Synthesis of 2-[(3R,8S)-15-(hydroxymethyl)-2,5-dioxa-9,15,17-triazatetracyclo[8.7.0.0\hat{ }{3,8}.0\hat{ }{12,16}]heptadeca-1(10),11,13,16-tetraen-9-yl]-4-{2-[(2S)-2-(2-isopropoxyphenyl)-4-[(4-methoxyphenyl)methyl]piperazin-1-yl]-7-azaspiro[3.5]nonan-7-yl}-N-[3-nitro-4-({[(1r,4r)-4-hydroxy-4-methylcyclohexyl]methyl}amino)benzenesulfonyl]benzamide (assumed): Into an 8 mL vial were added 2-[(3R,8S)-15-(hydroxymethyl)-2,5-dioxa-9,15,17-triazatetracyclo[8.7.0.0\hat{ }{3,8}.0\hat{ }{12,16}]heptadeca-1(10),11,13,16-tetraen-9-yl]-4-{2-[(2S)-2-(2-isopropoxyphenyl)piperazin-1-yl]-7-azaspiro[3.5]nonan-7-yl}-N-[3-nitro-4-({[(1r,4r)-4-hydroxy-4-methylcyclohexyl]methyl}amino)benzenesulfonyl]benzamide (100 mg, 0.1 mmol, 1.0 eq), anisaldehyde (20 mg, 0.1 mmol, 1.5 eq), NaBH₃CN (30 mg, 0.5 mmol, 5.0 eq), ZnCl₂ (65 mg, 0.5 mmol, 5.0 eq) and MeOH (3 mL). The resulting mixture was stirred for 4 hours at 25° C. The reaction was quenched by the addition of water (2 mL) and then extracted with CH₂Cl₂ (3×5 mL). The combined organic layer was washed with brine (5 mL), dried over anhydrous Na₂SO₄. After filtration, the filtrate was concentrated under vacuum. The crude residue was purified by Prep-TLC (CH₂Cl₂/MeOH=12:1) to give 2-[(3R,8S)-15-(hydroxymethyl)-2,5-dioxa-9,15,17-triazatetracyclo[8.7.0.0\hat{ }{3,8}.0\hat{ }{12,16}]heptadeca-1(10),11,13,16-tetraen-9-yl]-4-{2-[(2S)-2-(2-isopropoxyphenyl)-4-[(4-methoxyphenyl)methyl]piperazin-1-yl]-7-azaspiro[3.5]nonan-7-yl}-N-[3-nitro-4-({[(1r,4r)-4-hydroxy-4-methylcyclohexyl]methyl}amino)benzenesulfonyl]benzamide as a yellow solid (assumed) (70 mg, 62.8%). LC-MS (ESI, m/z) M+1: 1168.

Synthesis of 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-4-{2-[(2S)-2-(2-isopropoxyphenyl)-4-[(4-methoxyphenyl)methyl]piperazin-1-yl]-7-azaspiro[3.5]nonan-7-yl}-N-[3-nitro-4-({[(1r,4r)-4-hydroxy-4-methylcyclohexyl]methyl}amino)benzenesulfonyl]benzamide (assumed): Into an 8 mL vial were added 2-[(3R,8S)-15-(hydroxymethyl)-2,5-dioxa-9,15,17-triazatetracyclo[8.7.0.0\hat{ }{3,8}.0\hat{ }{12,16}]heptadeca-1(10),11,13,16-tetraen-9-yl]-4-{2-[(2S)-2-(2-isopropoxyphenyl)-4-[(4-methoxyphenyl)methyl]piperazin-1-yl]-7-azaspiro[3.5]nonan-7-yl}-N-[3-nitro-4-({[(1r,4r)-4-hydroxy-4-methylcyclohexyl]methyl}amino)benzenesulfonyl]benzamide (65 mg, 0.06 mmol, 1.0 eq), ethylenediamine (100 mg, 1.7 mmol, 30.0 eq) and THE (1 mL). The resulting mixture was stirred for 2 hours at 70° C. The reaction was quenched by the addition of water (3 mL) and then extracted with CH₂Cl₂ (3×3 mL). The combined organic layer was washed with brine (3 mL), dried over anhydrous Na₂SO₄. After filtration, the filtrate was concentrated under vacuum. The residue was purified by Prep-TLC (CH₂Cl₂/MeOH=0:1) to give 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-4-{2-[(2S)-2-(2-isopropoxyphenyl)-4-[(4-methoxyphenyl)methyl]piperazin-1-yl]-7-azaspiro[3.5]nonan-7-yl}-N-[3-nitro-4-({[(1r,4r)-4-hydroxy-4-methylcyclohexyl]methyl}amino)benzenesulfonyl]benzamide (assumed) as a yellow solid (16 mg, 25.3%). LC-MS (ESI, m/z) M+1: 1139. ¹H NMR (300 MHz, Methanol-d₄) δ 8.48 (d, J=2.2 Hz, 1H), 8.11 (s, 1H), 7.88 (d, J=9.1 Hz, 2H), 7.62 (d, J=8.7 Hz, 1H), 7.58-7.36 (m, 2H), 7.27 (d, J=8.5 Hz, 2H), 7.08 (d, J=3.4 Hz, 1H), 7.04-6.83 (m, 3H), 6.69 (d, J=9.4 Hz, 2H), 6.62-6.41 (m, 1H), 6.11-5.89 (m, 1H), 4.62 (d, J=12.8 Hz, 2H), 4.30 (d, J=7.2 Hz, 3H), 3.93 (d, J=13.1 Hz, 1H), 3.79 (d, J=2.1 Hz, 2H), 3.66 (s, 2H), 3.60-3.38 (m, 2H), 3.26 (d, J=6.8 Hz, 3H), 3.14 (d, J=16.1 Hz, 4H), 2.93-2.70 (m, 2H), 2.52 (s, 3H), 2.35-2.15 (m, 1H), 2.13-1.92 (m, 2H), 1.84 (d, J=13.5 Hz, 2H), 1.71 (s, 3H), 1.62-1.42 (m, 5H), 1.42-1.29 (m, 8H), 1.29-1.11 (m, 7H), 0.91 (d, J=7.1 Hz, 2H).

### Example 100 Preparation of 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-4-{2-[(2S)-2-(2-isopropoxyphenyl)-4-{[6-(morpholin-4-yl)pyridin-3-yl]methyl}piperazin-1-yl]-7-azaspiro[3.5]nonan-7-yl}-N-[3-nitro-4-({[(1r,4r)-4-hydroxy-4-methylcyclohexyl]methyl}amino)benzenesulfonyl]benzamide (assumed)

Synthesis of 6-(morpholin-4-yl)pyridine-3-carbaldehyde: Into a 50 mL round-bottom flask, were placed 6-fluoropyridine-3-carbaldehyde (3.0 g, 23.9 mmol, 1.0 eq), morpholine (4.2 g, 47.9 mmol, 2.0 eq), K₂CO₃ (6.6 g, 47.9 mmol, 2.0 eq), DMSO (45 mL). The resulting solution was stirred for 3 hours at 130° C. The reaction was then quenched by the addition of water (50 mL) and extracted with ethyl acetate (2×50 mL). The combined organic layer was washed with water (2×50 mL), brine (2×50 mL) and dried over anhydrous Na₂SO₄. After filtration, the filtrate was concentrated under vacuum. The crude residue was applied onto a silica gel column and eluted with ethyl acetate/petroleum ether=1:1 to give 6-(morpholin-4-yl)pyridine-3-carbaldehyde as a light yellow solid (4.0 g, 86.8%). ¹H NMR (300 MHz, DMSO-d₆) δ 9.76 (s, 1H), 8.61 (d, J=2.4 Hz, 1H), 7.90 (dd, J=9.0, 2.4 Hz, 1H), 6.94 (d, J=9.0 Hz, 1H), 3.69 (s, 8H).

Synthesis of 4-(5-{[(3S)-3-(2-isopropoxyphenyl)piperazin-1-yl]methyl}pyridin-2-yl)morpholine (assumed): Into an 8-mL sealed-tube, were placed (2S)-2-(2-isopropoxyphenyl)piperazine (70 mg, 0.3 mmol, 1.0 eq), 6-(morpholin-4-yl)pyridine-3-carbaldehyde (49 mg, 0.3 mmol, 0.8 eq), DCE (2 mL), NaBH(OAc)₃ (202 mg, 0.9 mmol, 3.0 eq). The resulting solution was stirred for 2 hours at 25° C. The reaction was then quenched by the addition of water (20 mL) and extracted with dichloromethane/methanol=20:1 (2×20 mL). The combined organic layer was washed with brine (2×40 mL) and dried over anhydrous Na₂SO₄. After filtration, the filtrate was concentrated under vacuum. The crude residue was purified onto a silica gel column and eluted with dichloromethane/methanol=10:1 to give 4-(5-{[(3S)-3-(2-isopropoxyphenyl)piperazin-1-yl]methyl}pyridin-2-yl)morpholine (assumed) as an off-white solid (85 mg, 67.5%). LC-MS (ESI, m/z) M+1: 397.

Synthesis of 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-4-{2-[(2S)-2-(2-isopropoxyphenyl)-4-{[6-(morpholin-4-yl)pyridin-3-yl]methyl}piperazin-1-yl]-7-azaspiro[3.5]nonan-7-yl}-N-[3-nitro-4-({[(1r,4r)-4-hydroxy-4-methylcyclohexyl]methyl}amino)benzenesulfonyl]benzamide (assumed): Into an 8-mL sealed-tube, were placed 4-(5-{[(3S)-3-(2-isopropoxyphenyl)piperazin-1-yl]methyl}pyridin-2-yl)morpholine (39 mg, 0.1 mmol, 1.0 eq), 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-N-[3-nitro-4-({[(1r,4r)-4-hydroxy-4-methylcyclohexyl]methyl}amino)benzenesulfonyl]-4-{2-oxo-7-azaspiro[3.5]nonan-7-yl}benzamide (80 mg, 0.1 mmol, 1.0 eq), MeOH (1 mL), ZnCl₂ (40 mg, 0.3 mmol, 3.0 eq), NaBH₃CN (19 mg, 0.3 mmol, 3.0 eq). The resulting solution was stirred for 4 hours at 70° C. The reaction was then quenched by the addition of water (20 mL) and extracted with dichloromethane/methanol=10:1 (2×30 mL). The combined organic layer was washed with brine (2×20 mL) and dried over anhydrous Na₂SO₄. After filtration, the filtrate was concentrated under vacuum. The residue was purified by Prep-TLC (dichloromethane/methanol=15:1) to give 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-4-{2-[(2S)-2-(2-isopropoxyphenyl)-4-{[6-(morpholin-4-yl)pyridin-3-yl]methyl}piperazin-1-yl]-7-azaspiro[3.5]nonan-7-yl}-N-[3-nitro-4-({[(1r,4r)-4-hydroxy-4-methylcyclohexyl]methyl}amino)benzenesulfonyl]benzamide (assumed) as a light yellow solid (25 mg, 21.3%). LC-MS (ESI, m/z) M+1: 1195. ¹H NMR (400 MHz, Methanol-d₄) δ 8.50 (dd, J=9.4, 2.4 Hz, 2H), 8.04 (d, J=2.4 Hz, 1H), 7.87 (d, J=9.2 Hz, 1H), 7.66-7.54 (m, 1H), 7.49 (dd, J=9.2, 2.4 Hz, 1H), 7.46-7.40 (m, 1H), 7.23 (t, J=8.0 Hz, 1H), 7.07 (d, J=3.5 Hz, 1H), 6.96-6.88 (m, 2H), 6.83-6.80 (m, 1H), 6.69-6.65 (m, 2H), 6.49 (s, 1H), 6.02 (d, J=3.4 Hz, 1H), 4.66-4.57 (m, 1H), 4.29 (d, J=7.6 Hz, 1H), 4.12 (br, 1H), 3.91 (d, J=11.3 Hz, 1H), 3.82-3.75 (m, 4H), 3.70 (br, 2H), 3.56-3.44 (m, 9H), 3.29-3.24 (m, 2H), 3.18-3.09, (m, 7H), 3.00-2.97 (m, 1H), 2.72-2.66 (m, 1H), 2.50-2.32 (m, 2H), 1.98-1.82 (m, 2H), 1.75-1.66 (m, 4H), 1.56-1.49 (m, 4H), 1.45-1.36 (m, 6H), 1.32-1.25 (m, 11H).

### Example 101 Preparation of N-((4-((((1r,4r)-4-hydroxy-4-methylcyclohexyl)methyl)amino)-3-nitrophenyl)sulfonyl)-4-(2-((S)-2-(2-isopropoxyphenyl)-4-((5-methoxy-6-morpholinopyridin-3-yl)methyl)piperazin-1-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide (assumed)

Synthesis of 4-(5-{[(3S)-4-benzyl-3-(2-isopropoxyphenyl)piperazin-1-yl]methyl}-3-methoxypyridin-2-yl)morpholine (assumed): Into a 40-mL sealed-tube, were placed (2S)-1-benzyl-2-(2-isopropoxyphenyl)piperazine (assumed) (150 mg, 0.5 mmol, 1.0 eq), 5-methoxy-6-morpholinonicotinaldehyde (118 mg, 0.5 mmol, 1.1 eq), DCE (4 mL), NaBH(OAc)₃ (307 mg, 1.4 mmol, 3.0 eq). The resulting solution was stirred for 2 hours at 25° C. The reaction was then quenched by the addition of water (40 mL) and extracted with ethyl acetate (2×40 mL). The combined organic layer was washed with brine (2×40 mL) and dried over anhydrous Na₂SO₄. After filtration, the filtrate was concentrated under vacuum. The crude residue was purified onto a silica gel column and eluted with ethyl acetate/petroleum ether=1:2 to give 4-(5-{[(3S)-4-benzyl-3-(2-isopropoxyphenyl)piperazin-1-yl]methyl}-3-methoxypyridin-2-yl)morpholine (assumed) as a light yellow solid (110 mg, 44.1%). LC-MS (ESI, m/z) M+1: 517.

Synthesis of 4-(5-{[(3S)-3-(2-isopropoxyphenyl)piperazin-1-yl]methyl}-3-methoxypyridin-2-yl)morpholine (assumed): Into a 50-mL round-bottom flask, were placed 4-(5-{[(3S)-4-benzyl-3-(2-isopropoxyphenyl)piperazin-1-yl]methyl}-3-methoxypyridin-2-yl)morpholine (assumed) (90 mg, 0.2 mmol, 1.0 eq), THE (3 mL), Pd/C (9 mg, 0.1 mmol, 0.5 eq), Pd(OH)₂/C (12 mg, 0.1 mmol, 0.5 eq). The mixture was hydrogenated at 25° C. under H₂ (2 atm) for 8 hours. The resulting mixture was filtered and the filtrate was concentrated under vacuum to give 4-(5-{[(3S)-3-(2-isopropoxyphenyl)piperazin-1-yl]methyl}-3-methoxypyridin-2-yl)morpholine (assumed) as a light yellow oil (70 mg, crude). LC-MS (ESI, m/z) M+1: 427.

Synthesis of N-((4-((((1r,4r)-4-hydroxy-4-methylcyclohexyl)methyl)amino)-3-nitrophenyl)sulfonyl)-4-(2-((S)-2-(2-isopropoxyphenyl)-4-((5-methoxy-6-morpholinopyridin-3-yl)methyl)piperazin-1-yl)-7-azaspiro[3.5]nonan-7-yl)-2-((5aS,9aR)-1-((2-(trimethylsilyl)ethoxy)methyl)-6,7,9,9a-tetrahydro-1H-pyrano[3,4-b]pyrrolo[3′,2′:5,6]pyrido[3,2-e][1,4]oxazin-5(5aH)-yl)benzamide (assumed): Into an 8 mL vial, were added N-[3-nitro-4-({[(1r,4r)-4-hydroxy-4-methylcyclohexyl]methyl}amino)benzenesulfonyl]-4-{2-oxo-7-azaspiro[3.5]nonan-7-yl}-2-[(3R,8S)-15-{[2-(trimethylsilyl)ethoxy]methyl}-2,5-dioxa-9,15,17-triazatetracyclo[8.7.0.0\hat{ }{3,8}.0\hat{ }{12,16}]heptadeca-1(10),11,13,16-tetraen-9-yl]benzamide (87 mg, 0.1 mmol, 0.7 eq), 4-(5-{[(3S)-3-(2-isopropoxyphenyl)piperazin-1-yl]methyl}-3-methoxypyridin-2-yl)morpholine (assumed) (45 mg, 0.1 mmol, 1.0 eq), NaBH₃CN (41 mg, 0.7 mmol, 5.0 eq), ZnCl₂ (90 mg, 0.7 mmol, 5.0 eq) and MeOH (2 mL). The resulting mixture was stirred for 5 hours at 70° C. The reaction was quenched by the addition of water (2 mL) and then extracted with CH₂Cl₂ (3×3 mL). The combined organic layer was washed with brine (3 mL), dried over anhydrous Na₂SO₄. After filtration, the filtrate was concentrated under vacuum. The residue was purified by Prep-TLC (CH₂Cl₂/MeOH=20:1) to give N-((4-((((1r,4r)-4-hydroxy-4-methylcyclohexyl)methyl)amino)-3-nitrophenyl)sulfonyl)-4-(2-((S)-2-(2-isopropoxyphenyl)-4-((5-methoxy-6-morpholinopyridin-3-yl)methyl)piperazin-1-yl)-7-azaspiro[3.5]nonan-7-yl)-2-((5aS,9aR)-1-((2-(trimethylsilyl)ethoxy)methyl)-6,7,9,9a-tetrahydro-1H-pyrano[3,4-b]pyrrolo[3′,2′:5,6]pyrido[3,2-e][1,4]oxazin-5(5aH)-yl)benzamide (assumed) as a yellow solid (35 mg, 20.9%). LC-MS (ESI, m/z) M+1: 1355.

Synthesis of N-((4-((((1r,4r)-4-hydroxy-4-methylcyclohexyl)methyl)amino)-3-nitrophenyl)sulfonyl)-4-(2-((S)-2-(2-isopropoxyphenyl)-4-((5-methoxy-6-morpholinopyridin-3-yl)methyl)piperazin-1-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide (assumed): Into an 8 mL vial were added 4-{2-[(2S)-2-(2-isopropoxyphenyl)-4-{[5-methoxy-6-(morpholin-4-yl)pyridin-3-yl]methyl}piperazin-1-yl]-7-azaspiro[3.5]nonan-7-yl}-N-[3-nitro-4-({[(1r,4r)-4-hydroxy-4-methylcyclohexyl]methyl}amino)benzenesulfonyl]-2-[(3R,8S)-15-{[2-(trimethylsilyl)ethoxy]methyl}-2,5-dioxa-9,15,17-triazatetracyclo[8.7.0.0\hat{ }{3,8}.0\hat{ }{12,16}]heptadeca-1(10),11,13,16-tetraen-9-yl]benzamide (assumed) (33 mg, 0.03 mmol, 1.0 eq), ethylenediamine (31 mg, 0.5 mmol, 20.0 eq) and TBAF in THE (1 M, 1 mL). The resulting mixture was stirred for 6 hours at 80° C. The reaction was quenched by the addition of water (3 mL) and then extracted with CH₂Cl₂ (3×3 mL). The combined organic layer was washed with brine (5×3 mL), dried over anhydrous Na₂SO₄. After filtration, the filtrate was concentrated under vacuum. After filtration, the filtrate was concentrated under vacuum. The residue was purified by Prep-TLC (CH₂Cl₂/MeOH=20:1) to give N-((4-((((1r,4r)-4-hydroxy-4-methylcyclohexyl)methyl)amino)-3-nitrophenyl)sulfonyl)-4-(2-((S)-2-(2-isopropoxyphenyl)-4-((5-methoxy-6-morpholinopyridin-3-yl)methyl)piperazin-1-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide (assumed) as a yellow solid (10.5 mg, 36.3%). LC-MS (ESI, m/z) M+1: 1225. ¹H NMR (300 MHz, Chloroform-d) δ 12.58 (s, 1H), 8.65 (d, J-2.1 Hz, 1H), 8.46 (s, 2H), 8.11 (d, J=9.3 Hz, 1H), 7.81 (d, J=8.4 Hz, 2H), 7.10 (d, J=3.0 Hz, 1H), 6.98 (s, 1H), 6.83 (d, J=9.3 Hz, 3H), 6.71 (d, J=9.3 Hz, 1H), 6.63 (s, 1H), 6.43 (s, 1H), 6.11 (s, 1H), 4.54 (s, 4H), 4.52-4.41 (m, 1H), 3.98 (d, J=10.2 Hz, 2H), 3.86 (s, 9H), 3.58 (s, 2H), 3.50 (t, J=10.2 Hz, 4H), 3.41 (d, J=17.5 Hz, 5H), 3.21 (t, J=6.0 Hz, 2H), 2.76 (s, 1H), 2.36 (s, 3H), 2.06 (s, 1H), 1.89 (s, 2H), 1.56-1.46 (m, 1H), 1.44 (d, J=22.2 Hz, 1H), 1.19 (d, J=12.5 Hz, 12H), 0.88 (s, 3H), 0.09 (s, 12H).

### Example 102 Preparation of 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-4-{2-[(2S)-2-(2-isopropoxyphenyl)-4-[(6-methoxypyridin-3-yl)methyl]piperazin-1-yl]-7-azaspiro[3.5]nonan-7-yl}-N-[3-nitro-4-({[(1r,4r)-4-hydroxy-4-methylcyclohexyl]methyl}amino)benzenesulfonyl]benzamide (assumed)

Synthesis of tert-butyl (3S)-3-(2-isopropoxyphenyl)-4-[7-(4-{[3-nitro-4-({[(1r,4r)-4-hydroxy-4-methylcyclohexyl]methyl}amino)benzenesulfonyl]carbamoyl}-3-[(3R,8S)-15-{[2-(trimethylsilyl)ethoxy]methyl}-2,5-dioxa-9,15,17-triazatetracyclo[8.7.0.0\hat{ }{3,8}.0\hat{ }{12,16}]heptadeca-1(10),11,13,16-tetraen-9-yl]phenyl)-7-azaspiro[3.5]nonan-2-yl]piperazine-1-carboxylate (assumed): Into a 40 mL vial were added N-[3-nitro-4-({[(1r,4r)-4-hydroxy-4-methylcyclohexyl]methyl}amino)benzenesulfonyl]-4-{2-oxo-7-azaspiro[3.5]nonan-7-yl}-2-[(3R,8S)-15-{[2-(trimethylsilyl)ethoxy]methyl}-2,5-dioxa-9,15,17-triazatetracyclo[8.7.0.0\hat{ }{3,8}.0\hat{ }{12,16}]heptadeca-1(10),11,13,16-tetraen-9-yl]benzamide (800 mg, 0.8 mmol, 1.0 eq), tert-butyl (3S)-3-(2-isopropoxyphenyl)piperazine-1-carboxylate (272 mg, 0.8 mmol, 1.0 eq), NaBH₃CN (266 mg, 4.2 mmol, 5.0 eq), ZnCl₂ (577 mg, 4.2 mmol, 5.0 eq) and MeOH (10 mL). The resulting mixture was stirred for 2 hours at 70° C. The reaction was quenched by the addition of water (5 mL) and then extracted with CH₂Cl₂ (3×20 mL). The combined organic layer was washed with brine (20 mL), dried over anhydrous Na₂SO₄. After filtration, the filtrate was concentrated under vacuum. The crude residue was purified by silica gel column chromatography and eluted with CH₂Cl₂/MeOH=30:1 to give tert-butyl (3S)-3-(2-isopropoxyphenyl)-4-[7-(4-{[3-nitro-4-({[(1r,4r)-4-hydroxy-4-methylcyclohexyl]methyl}amino)benzenesulfonyl]carbamoyl}-3-[(3R,8S)-15-{[2-(trimethylsilyl)ethoxy]methyl}-2,5-dioxa-9,15,17-triazatetracyclo[8.7.0.0\hat{ }{3,8}.0\hat{ }{12,16}]heptadeca-1(10),11,13,16-tetraen-9-yl]phenyl)-7-azaspiro[3.5]nonan-2-yl]piperazine-1-carboxylate (assumed) as a yellow solid (800 mg, 75.6%). LC-MS (ESI, m/z) M+1: 1248.

Synthesis of 2-[(3R,8S)-15-(hydroxymethyl)-2,5-dioxa-9,15,17-triazatetracyclo[8.7.0.0\hat{ }{3,8}.0\hat{ }{12,16}]heptadeca-1(10),11,13,16-tetraen-9-yl]-4-{2-[(2S)-2-(2-isopropoxyphenyl)piperazin-1-yl]-7-azaspiro[3.5]nonan-7-yl}-N-[3-nitro-4-({[(1r,4r)-4-hydroxy-4-methylcyclohexyl]methyl}amino)benzenesulfonyl]benzamide (assumed): Into a 50 mL round-bottom flask were added tert-butyl (3S)-3-(2-isopropoxyphenyl)-4-[7-(4-{[3-nitro-4-({[(1r,4r)-4-hydroxy-4-methylcyclohexyl]methyl}amino)benzenesulfonyl]carbamoyl}-3-[(3R,8S)-15-{[2-(trimethylsilyl)ethoxy]methyl}-2,5-dioxa-9,15,17-triazatetracyclo[8.7.0.0\hat{ }{3,8}.0\hat{ }{12,16}]heptadeca-1(10),11,13,16-tetraen-9-yl]phenyl)-7-azaspiro[3.5]nonan-2-yl]piperazine-1-carboxylate (600 mg, 0.5 mmol, 1.0 eq) and TFA/DCM(3 mL/3 mL) at 0° C. The resulting mixture was stirred for 2 hours at 0° C. The mixture was basified to pH=8 with sat. NaHCO₃ and then extracted with CH₂Cl₂ (3×10 mL). The combined organic layer was washed with brine (10 mL), dried over anhydrous Na₂SO₄. After filtration, the filtrate was concentrated under vacuum. The crude residue was purified by Prep-TLC (CH₂Cl₂/MeOH=8:1) to give 2-[(3R,8S)-15-(hydroxymethyl)-2,5-dioxa-9,15,17-triazatetracyclo[8.7.0.0\hat{ }{3,8}.0\hat{ }{12,16}]heptadeca-1(10),11,13,16-tetraen-9-yl]-4-{2-[(2S)-2-(2-isopropoxyphenyl)piperazin-1-yl]-7-azaspiro[3.5]nonan-7-yl}-N-[3-nitro-4-({[(1r,4r)-4-hydroxy-4-methylcyclohexyl]methyl}amino)benzenesulfonyl]benzamide (assumed) as a yellow solid (280 mg, 55.6%). LC-MS (ESI, m/z) M+1: 1048.

Synthesis of 2-[(3R,8S)-15-(hydroxymethyl)-2,5-dioxa-9,15,17-triazatetracyclo[8.7.0.0\hat{ }{3,8}.0\hat{ }{12,16}]heptadeca-1(10),11,13,16-tetraen-9-yl]-4-{2-[(2S)-2-(2-isopropoxyphenyl)-4-[(6-methoxypyridin-3-yl)methyl]piperazin-1-yl]-7-azaspiro[3.5]nonan-7-yl}-N-[3-nitro-4-({[(1r,4r)-4-hydroxy-4-methylcyclohexyl]methyl}amino)benzenesulfonyl]benzamide (assumed): Into an 8 mL vial were added 2-[(3R,8S)-15-(hydroxymethyl)-2,5-dioxa-9,15,17-triazatetracyclo[8.7.0.0\hat{ }{3,8}.0\hat{ }{12,16}]heptadeca-1(10),11,13,16-tetraen-9-yl]-4-{2-[(2S)-2-(2-isopropoxyphenyl)piperazin-1-yl]-7-azaspiro[3.5]nonan-7-yl}-N-[3-nitro-4-({[(1r,4r)-4-hydroxy-4-methylcyclohexyl]methyl}amino)benzenesulfonyl]benzamide (100 mg, 0.1 mmol, 1.0 eq), NaBH₃CN (30 mg, 0.5 mmol, 5.0 eq), ZnCl₂ (65 mg, 0.5 mmol, 5.0 eq) and MeOH (2 mL). The resulting mixture was stirred for 4 hours. The reaction was quenched by the addition of water (3 mL) and then extracted with CH₂Cl₂ (3×5 mL). The combined organic layer was washed with brine (5 mL), dried over anhydrous Na₂SO₄. After filtration, the filtrate was concentrated under vacuum. The crude residue was purified by Prep-TLC (CH₂Cl₂/MeOH=12:1) to give 2-[(3R,8S)-15-(hydroxymethyl)-2,5-dioxa-9,15,17-triazatetracyclo[8.7.0.0\hat{ }{3,8}.0\hat{ }{12,16}]heptadeca-1(10),11,13,16-tetraen-9-yl]-4-{2-[(2S)-2-(2-isopropoxyphenyl)-4-[(6-methoxypyridin-3-yl)methyl]piperazin-1-yl]-7-azaspiro[3.5]nonan-7-yl}-N-[3-nitro-4-({[(1r,4r)-4-hydroxy-4-methylcyclohexyl]methyl}amino)benzenesulfonyl]benzamide (assumed) as a yellow solid (65 mg, 58.3%). LC-MS (ESI, m/z) M+1: 1170.

Synthesis of 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-4-{2-[(2S)-2-(2-isopropoxyphenyl)-4-[(6-methoxypyridin-3-yl)methyl]piperazin-1-yl]-7-azaspiro[3.5]nonan-7-yl}-N-[3-nitro-4-({[(1r,4r)-4-hydroxy-4-methylcyclohexyl]methyl}amino)benzenesulfonyl]benzamide (assumed): Into an 8 mL vial were added 2-[(3R,8S)-15-(hydroxymethyl)-2,5-dioxa-9,15,17-triazatetracyclo[8.7.0.0\hat{ }{3,8}.0\hat{ }{12,16}]heptadeca-1(10),11,13,16-tetraen-9-yl]-4-{2-[(2S)-2-(2-isopropoxyphenyl)-4-[(6-methoxypyridin-3-yl)methyl]piperazin-1-yl]-7-azaspiro[3.5]nonan-7-yl}-N-[3-nitro-4-({[(1r,4r)-4-hydroxy-4-methylcyclohexyl]methyl}amino)benzenesulfonyl]benzamide (60 mg, 0.05 mmol, 1.0 eq) ethylenediamine (92 mg, 1.5 mmol, 30.0 eq) and THE (1 mL). The resulting mixture was stirred for 2 hours at 70° C. The reaction was quenched by the addition of water (3 mL) and then extracted with CH₂Cl₂ (3×3 mL). The combined organic layer was washed with brine (3 mL), dried over anhydrous Na₂SO₄. After filtration, the filtrate was concentrated under vacuum. The residue was purified by Prep-TLC (CH₂Cl₂/MeOH=25:1) to give 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-4-{2-[(2S)-2-(2-isopropoxyphenyl)-4-[(6-methoxypyridin-3-yl)methyl]piperazin-1-yl]-7-azaspiro[3.5]nonan-7-yl}-N-[3-nitro-4-({[(1r,4r)-4-hydroxy-4-methylcyclohexyl]methyl}amino)benzenesulfonyl]benzamide (assumed) as a yellow solid (12 mg, 20.53%). LC-MS (ESI, m/z) M+1: 1140. ¹H NMR (300 MHz, Methanol-d₄) δ 8.49 (d, J=2.3 Hz, 1H), 8.13-8.00 (m, 1H), 7.86 (d, J=9.3 Hz, 1H), 7.80-7.61 (m, 1H), 7.61-7.38 (m, 2H), 7.23 (t, J=7.6 Hz, 2H), 7.07 (d, J=3.4 Hz, 1H), 7.03-6.89 (m, 2H), 6.89-6.77 (m, 1H), 6.77-6.63 (m, 1H), 6.51 (d, J=13.8 Hz, 1H), 5.99 (dd, J=17.5, 3.4 Hz, 2H), 4.60 (d, J=5.6 Hz, 1H), 4.28 (d, J=7.6 Hz, 2H), 4.08 (d, J=24.3 Hz, 1H), 3.90 (d, J=2.0 Hz, 4H), 3.70 (s, 3H), 3.64-3.41 (m, 3H), 3.25 (d, J=4.8 Hz, 7H), 2.92 (d, J=28.4 Hz, 1H), 2.66 (d, J=12.4 Hz, 3H), 2.39 (s, 2H), 2.20 (d, J=16.0 Hz, 1H), 1.96 (s, 1H), 1.84 (d, J=14.0 Hz, 2H), 1.71 (s, 3H), 1.64-1.45 (m, 4H), 1.45-1.35 (m, 2H), 1.32 (d, J=5.6 Hz, 6H), 1.25 (t, J=3.3 Hz, 6H), 1.00-0.85 (m, 2H).

### Example 103 Preparation of 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-4-{2-[(2S)-2-(2-isopropoxyphenyl)-4-[(5-methoxypyridin-2-yl)methyl]piperazin-1-yl]-7-azaspiro[3.5]nonan-7-yl}-N-[3-nitro-4-({[(1r,4r)-4-hydroxy-4-methylcyclohexyl]methyl}amino)benzenesulfonyl]benzamide (assumed)

Synthesis of (2S)-1-benzyl-2-(2-isopropoxyphenyl)-4-[(5-methoxypyridin-2-yl)methyl]piperazine (assumed): Into a 40 mL vial were added (2S)-1-benzyl-2-(2-isopropoxyphenyl)piperazine (150 mg, 0.5 mmol, 1.0 eq), 5-methoxypyridine-2-carbaldehyde (80 mg, 0.6 mmol, 1.2 eq), ZnCl₂ (329 mg, 2.4 mmol, 5.0 eq) and MeOH (5 mL). The resulting mixture was stirred for 30 min at 25° C. To the above mixture was added NaBH₃CN (152 mg, 2.4 mmol, 5.0 eq) at 25° C. The resulting mixture was stirred for additional 2 hours at 25° C. The reaction was quenched by the addition of water (5 mL) and then extracted with EtOAc (3×5 mL). The combined organic layer was washed with brine (5 mL), dried over anhydrous Na₂SO₄. After filtration, the filtrate was concentrated under vacuum. The residue was applied onto a silica gel column and eluted with ethyl acetate/petroleum ether=1:20 to give (2S)-1-benzyl-2-(2-isopropoxyphenyl)-4-[(5-methoxypyridin-2-yl)methyl]piperazine (assumed) as a colorless oil (100 mg, 47.9%). LC-MS (ESI, m/z) M+1: 432.

Synthesis of (3S)-3-(2-isopropoxyphenyl)-1-[(5-methoxypyridin-2-yl)methyl]piperazine: Into a 50 mL round-bottom flask were added (2S)-1-benzyl-2-(2-isopropoxyphenyl)-4-[(5-methoxypyridin-2-yl)methyl]piperazine (80 mg, 0.19 mmol, 1.0 eq), Pd(OH)₂/C (13 mg, 0.1 mmol, 0.5 eq), Pd/C (10 mg, 0.01 mmol, 0.5 eq) and MeOH (2 mL). The resulting mixture was stirred for 3 hours at 0° C. under H₂ (1 atm). The resulting mixture was filtered, the filter cake was washed with MeOH (3×5 mL). The filtrate was concentrated under vacuum to give (3S)-3-(2-isopropoxyphenyl)-1-[(5-methoxypyridin-2-yl)methyl]piperazine as a colorless oil (50 mg, crude), which was used in the next step directly without further purification to give. ¹H NMR (300 MHz, Chloroform-d) δ 8.26 (d, J=3.0 Hz, 1H), 7.71 (s, 1H), 7.16 (dd, J=8.4, 3.1 Hz, 3H), 6.96 (t, J=7.6 Hz, 1H), 6.87 (d, J=8.2 Hz, 1H), 4.56 (p, J=6.1 Hz, 1H), 4.10 (s, 1H), 3.85 (s, 3H), 3.64 (s, 2H), 2.89 (d, J=12.1 Hz, 2H), 2.30-2.52 (m. 2H), 2.05 (s, 2H), 1.37 (d, J=6.1 Hz, 3H), 1.28 (d, J=4.4 Hz, 3H).

Synthesis of 4-{2-[(2S)-2-(2-isopropoxyphenyl)-4-[(5-methoxypyridin-2-yl)methyl]piperazin-1-yl]-7-azaspiro[3.5]nonan-7-yl}-N-[3-nitro-4-({[(1r,4r)-4-hydroxy-4-methylcyclohexyl]methyl}amino)benzenesulfonyl]-2-[(3R,8S)-15-{[2-(trimethylsilyl)ethoxy]methyl}-2,5-dioxa-9,15,17-triazatetracyclo[8.7.0.0\hat{ }{3,8}.0\hat{ }{12,16}]heptadeca-1(10),11,13,16-tetraen-9-yl]benzamide (assumed): Into an 8 mL vial were added N-[3-nitro-4-({[(1r,4r)-4-hydroxy-4-methylcyclohexyl]methyl}amino)benzenesulfonyl]-4-{2-oxo-7-azaspiro[3.5]nonan-7-yl}-2-[(3R,8S)-15-{[2-(trimethylsilyl)ethoxy]methyl}-2,5-dioxa-9,15,17-triazatetracyclo[8.7.0.0\hat{ }{3,8}.0\hat{ }{12,16}]heptadeca-1(10),11,13,16-tetraen-9-yl]benzamide (87 mg, 0.1 mmol, 0.7 eq), (3S)-3-(2-isopropoxyphenyl)-1-[(5-methoxypyridin-2-yl)methyl]piperazine (45 mg, 0.1 mmol, 1.0 eq), NaBH₃CN (41 mg, 0.7 mmol, 5.0 eq), ZnCl₂ (90 mg, 0.7 mmol, 5 eq) and MeOH (2 mL). The resulting mixture was stirred for 2 hours at 70° C. The reaction was quenched by the addition of water (2 mL) and extracted with CH₂Cl₂ (3×3 mL). The combined organic layer was washed with brine (3 mL), and then dried over anhydrous Na₂SO₄. After filtration, the filtrate was concentrated under vacuum. The residue was purified by Prep-TLC (CH₂Cl₂/MeOH=20:1) to give 4-{2-[(2S)-2-(2-isopropoxyphenyl)-4-[(5-methoxypyridin-2-yl)methyl]piperazin-1-yl]-7-azaspiro[3.5]nonan-7-yl}-N-[3-nitro-4-({[(1r,4r)-4-hydroxy-4-methylcyclohexyl]methyl}amino)benzenesulfonyl]-2-[(3R,8S)-15-{[2-(trimethylsilyl)ethoxy]methyl}-2,5-dioxa-9,15,17-triazatetracyclo[8.7.0.0\hat{ }{3,8}.0\hat{ }{12,16}]heptadeca-1(10),11,13,16-tetraen-9-yl]benzamide (assumed) as a yellow solid (35 mg, 20.9%). LC-MS (ESI, m/z) M+1: 1269.

Synthesis of 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-4-{2-[(2S)-2-(2-isopropoxyphenyl)-4-[(5-methoxypyridin-2-yl)methyl]piperazin-1-yl]-7-azaspiro[3.5]nonan-7-yl}-N-[3-nitro-4-({[(1r,4r)-4-hydroxy-4-methylcyclohexyl]methyl}amino)benzenesulfonyl]benzamide (assumed): Into an 8 mL vial were added 4-{2-[(2S)-2-(2-isopropoxyphenyl)-4-[(5-methoxypyridin-2-yl)methyl]piperazin-1-yl]-7-azaspiro[3.5]nonan-7-yl}-N-[3-nitro-4-({[(1r,4r)-4-hydroxy-4-methylcyclohexyl]methyl}amino)benzenesulfonyl]-2-[(3R,8S)-15-{[2-(trimethylsilyl)ethoxy]methyl}-2,5-dioxa-9,15,17-triazatetracyclo[8.7.0.0\hat{ }{3,8}.0\hat{ }{12,16}]heptadeca-1(10),11,13,16-tetraen-9-yl]benzamide (33 mg, 0.03 mmol, 1.0 eq), ethylenediamine (31 mg, 0.5 mmol, 20.0 eq) and TBAF in THF(1 M, 1 mL). The resulting mixture was stirred for 3 hours at 80° C. The reaction was quenched by the addition of water (3 mL) and then extracted with CH₂Cl₂ (3×3 mL). The combined organic layer was washed with brine (5×3 mL), dried over anhydrous Na₂SO₄. After filtration, the filtrate was concentrated under vacuum. The crude residue was purified by Prep-TLC (CH₂Cl₂/MeOH=12:1) to give crude product. The residue was purified by reverse flash chromatography using the following conditions: column, C18 silica gel; mobile phase, MeCN in water (0.1% FA), 10% to 50% gradient in 10 min; detector, UV 254 nm. Finally, 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-4-{2-[(2S)-2-(2-isopropoxyphenyl)-4-[(5-methoxypyridin-2-yl)methyl]piperazin-1-yl]-7-azaspiro[3.5]nonan-7-yl}-N-[3-nitro-4-({[(1r,4r)-4-hydroxy-4-methylcyclohexyl]methyl}amino)benzenesulfonyl]benzamide (assumed) (3.6 mg, 12.2%) was obtained as a yellow solid. LC-MS (ESI, m/z) M+1: 1140. ¹H NMR (300 MHz, Methanol-d₄) δ 8.48 (d, J=2.4 Hz, 2H), 8.09 (d, J=10.0 Hz, 1H), 7.92 (dd, J=8.9, 4.6 Hz, 2H), 7.68 (s, 1H), 7.54 (dd, J=9.2, 2.3 Hz, 1H), 7.43 (t, J=7.5 Hz, 1H), 7.11 (s, 2H), 7.04 (dd, J=14.8, 7.3 Hz, 3H), 6.81-6.69 (m, 2H), 6.53 (s, 1H), 4.56 (d, J=7.1 Hz, 3H), 4.31 (d, J=7.5 Hz, 3H), 4.13 (s, 3H), 4.04 (d, J=1.6 Hz, 4H), 3.66 (s, 3H), 3.50-3.44 (m, 4H), 3.28 (d, J=6.7 Hz, 6H), 3.16 (s, 4H), 2.31 (s, 1H), 1.85 (d, J=13.9 Hz, 3H), 1.72 (s, 4H), 1.69 (s, 3H), 1.67 (s, 3H), 1.53 (s, 3H), 1.48 (d, J=6.0 Hz, 2H), 1.41 (t, J=7.0 Hz, 1H), 1.31 (s, 2H), 1.26 (d, J=4.4 Hz, 3H).

### Example 104 Preparation of 4-{2-[(2S)-4-[(5,6-dimethoxypyridin-3-yl)methyl]-2-(2-isopropoxyphenyl)piperazin-1-yl]-7-azaspiro[3.5]nonan-7-yl}-2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-N-[3-nitro-4-({[(1r,4r)-4-hydroxy-4-methylcyclohexyl]methyl}amino)benzenesulfonyl]benzamide (assumed)

Synthesis of 5-bromo-2,3-dimethoxypyridine: Into a 50-mL round-bottom flask, were placed 2,3-dimethoxypyridine (8.0 g, 57.5 mmol, 1.0 eq), Br₂ (10.1 g, 63.2 mmol, 1.1 eq), dichloromethane (80 mL). The resulting mixture was stirred for 14 hours at 25° C. The reaction was then quenched by the addition of water (50 mL), and then extracted with ethyl acetate (50 mL). The organic layer was washed with water (2×50 mL) and brine (2×50 mL), and dried over Na₂SO₄. After filtration, the resulting mixture was concentrated under vacuum. The crude residue was applied onto a silica gel column and eluted with ethyl acetate/petroleum ether=1:2 to give 5-bromo-2,3-dimethoxypyridine as an off-white oil (5.0 g, 40.0%). LC-MS (ESI, m/z) M+1: 218. ¹H NMR (300 MHz, Chloroform-d) δ 7.73 (d, J=2.1 Hz, 1H), 7.09 (d, J=2.1 Hz, 1H), 3.95 (s, 3H), 3.83 (s, 3H).

Synthesis of 5,6-dimethoxypyridine-3-carbaldehyde: Into a 250 mL 3-necked round-bottom flask, were placed 5-bromo-2,3-dimethoxypyridine (4.7 g, 21.6 mmol, 1.0 eq), THE (50 mL). After that, n-BuLi (1.1 eq) was added dropwised at −78° C. The resulting mixture was stirred for 30 minutes at −78° C. To the above mixture was added DMF (2.2 g, 30.2 mmol, 1.4 eq) at −78° C. The resulting mixture was stirred for additional 30 minutes at −78° C. The reaction was then quenched by the addition of water (200 mL) and extracted with ethyl acetate (2×100 mL). The combined organic layer was washed with brine (2×100 mL). The mixture was dried over anhydrous Na₂SO₄. After filtration, the filtrate was concentrated under vacuum. The crude residue was purified onto a silica gel column and eluted with ethyl acetate/petroleum ether=1:1 to give 5,6-dimethoxypyridine-3-carbaldehyde as a light pink solid (2.7 g, 74.9%). ¹H NMR (300 MHz, DMSO-d₆) δ 9.95 (s, 1H), 8.35 (d, J=1.8 Hz, 1H), 7.53 (d, J=1.8 Hz, 1H), 3.99 (s, 3H), 3.87 (s, 3H).

Synthesis of (2S)-1-benzyl-4-[(5,6-dimethoxypyridin-3-yl)methyl]-2-(2-isopropoxyphenyl)piperazine (assumed): Into a 40-mL sealed-tube, were placed (2S)-1-benzyl-2-(2-isopropoxyphenyl)piperazine (150 mg, 0.5 mmol, 1.0 eq), 5,6-dimethoxypyridine-3-carbaldehyde (89 mg, 0.5 mmol, 1.1 eq), DCE (4 mL), NaBH(OAc)₃ (307 mg, 1.4 mmol, 3.0 eq). The resulting solution was stirred for 2 hours at 25° C. The reaction was then quenched by the addition of water (40 mL) and then extracted with ethyl acetate (2×40 mL). The combined organic layer was washed with brine (2×40 mL) and dried over anhydrous Na₂SO₄. After filtration, the filtrate was concentrated under vacuum. The crude residue was purified onto a silica gel column and eluted with ethyl acetate/petroleum ether=1:2 to give (2S)-1-benzyl-4-[(5,6-dimethoxypyridin-3-yl)methyl]-2-(2-isopropoxyphenyl)piperazine (assumed) as a light yellow oil (140 mg, 62.8%). LC-MS (ESI, m/z) M+1: 462.

Synthesis of (3S)-1-[(5,6-dimethoxypyridin-3-yl)methyl]-3-(2-isopropoxyphenyl)piperazine (assumed): Into a 50-mL round-bottom flask, were placed (2S)-1-benzyl-4-[(5,6-dimethoxypyridin-3-yl)methyl]-2-(2-isopropoxyphenyl)piperazine (120 mg, 0.3 mmol, 1.0 eq), THE (3 mL), Pd/C (14 mg, 0.1 mmol, 0.5 eq), Pd(OH)₂/C (18 mg, 0.1 mmol, 0.5 eq). The mixture was hydrogenated at 25° C. under H₂ (2 atm) for 4 hours. The resulting mixture was filtered. The filtrate was concentrated under vacuum to give (3S)-1-[(5,6-dimethoxypyridin-3-yl)methyl]-3-(2-isopropoxyphenyl)piperazine (assumed) as a light yellow oil (90 mg, crude).

Synthesis of 4-{2-[(2S)-4-[(5,6-dimethoxypyridin-3-yl)methyl]-2-(2-isopropoxyphenyl)piperazin-1-yl]-7-azaspiro[3.5]nonan-7-yl}-N-[3-nitro-4-({[(1r,4r)-4-hydroxy-4-methylcyclohexyl]methyl}amino)benzenesulfonyl]-2-[(3R,8S)-15-{[2-(trimethylsilyl)ethoxy]methyl}-2,5-dioxa-9,15,17-triazatetracyclo[8.7.0.0\hat{ }{3,8}.0\hat{ }{12,16}]heptadeca-1(10),11,13,16-tetraen-9-yl]benzamide (assumed): Into an 8-mL sealed-tube, were placed (3S)-1-[(5,6-dimethoxypyridin-3-yl)methyl]-3-(2-isopropoxyphenyl)piperazine (80 mg, 0.2 mmol, 1.0 eq), N-[3-nitro-4-({[(1r,4r)-4-hydroxy-4-methylcyclohexyl]methyl}amino)benzenesulfonyl]-4-{2-oxo-7-azaspiro[3.5]nonan-7-yl}-2-[(3R,8S)-15-{[2-(trimethylsilyl)ethoxy]methyl}-2,5-dioxa-9,15,17-triazatetracyclo[8.7.0.0\hat{ }{3,8}.0\hat{ }{12,16}]heptadeca-1(10),11,13,16-tetraen-9-yl]benzamide (163 mg, 0.2 mmol, 0.8 eq), MeOH (1 mL), ZnCl₂ (88 mg, 0.6 mmol, 3.0 eq), NaBH₃CN (41 mg, 0.6 mmol, 3.0 eq). The resulting solution was stirred for 3 hours at 70° C. The reaction was then quenched by the addition of water (20 mL) and then extracted with dichloromethane/methanol=10:1 (2×20 mL). The combined organic layer was washed with brine (2×20 mL) and dried over anhydrous Na₂SO₄. After filtration, the filtrate was concentrated under vacuum. The crude residue was purified onto a silica gel column and eluted with dichloromethane/methanol=15:1 to give 4-{2-[(2S)-4-[(5,6-dimethoxypyridin-3-yl)methyl]-2-(2-isopropoxyphenyl)piperazin-1-yl]-7-azaspiro[3.5]nonan-7-yl}-N-[3-nitro-4-({[(1r,4r)-4-hydroxy-4-methylcyclohexyl]methyl}amino)benzenesulfonyl]-2-[(3R,8S)-15-{[2-(trimethylsilyl)ethoxy]methyl}-2,5-dioxa-9,15,17-triazatetracyclo[8.7.0.0\hat{ }{3,8}.0\hat{ }{12,16}]heptadeca-1(10),11,13,16-tetraen-9-yl]benzamide (assumed) as a light yellow solid (55 mg, 19.7%). LC-MS (ESI, m/z) M+1: 1300.

Synthesis of 4-{2-[(2S)-4-[(5,6-dimethoxypyridin-3-yl)methyl]-2-(2-isopropoxyphenyl)piperazin-1-yl]-7-azaspiro[3.5]nonan-7-yl}-2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-N-[3-nitro-4-({[(1r,4r)-4-hydroxy-4-methylcyclohexyl]methyl}amino)benzenesulfonyl]benzamide (assumed): Into an 8 mL sealed-tube, were placed 4-{2-[(2S)-4-[(5,6-dimethoxypyridin-3-yl)methyl]-2-(2-isopropoxyphenyl)piperazin-1-yl]-7-azaspiro[3.5]nonan-7-yl}-N-[3-nitro-4-({[(1r,4r)-4-hydroxy-4-methylcyclohexyl]methyl}amino)benzenesulfonyl]-2-[(3R,8S)-15-{[2-(trimethylsilyl)ethoxy]methyl}-2,5-dioxa-9,15,17-triazatetracyclo[8.7.0.0\hat{ }{3,8}.0\hat{ }{12,16}]heptadeca-1(10),11,13,16-tetraen-9-yl]benzamide (50 mg, 0.04 mmol, 1.0 eq), ethylenediamine (69 mg, 1.1 mmol, 30.0 eq) and TBAF in THE (1 M, 2 mL). The resulting mixture was stirred for 8 hours at 70° C. The reaction was quenched by the addition of water (10 mL) and then extracted with EtOAc (3×30 mL). The combined organic layer was washed with brine (30 mL), dried over anhydrous Na₂SO₄. After filtration, the filtrate was concentrated under vacuum. The residue was purified by Prep-TLC (dichloromethane/methanol=15:1) to give 4-{2-[(2S)-4-[(5,6-dimethoxypyridin-3-yl)methyl]-2-(2-isopropoxyphenyl)piperazin-1-yl]-7-azaspiro[3.5]nonan-7-yl}-2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-N-[3-nitro-4-({[(1r,4r)-4-hydroxy- 4-methylcyclohexyl]methyl}amino)benzenesulfonyl]benzamide (assumed) as a yellow solid (17 mg, 37.8%). LC-MS (ESI, m/z) M+1: 1170. ¹H NMR (400 MHz, Methanol-d₄) δ 8.46 (d, J=2.4 Hz, 1H), 7.93 (d, J=9.2 Hz, 1H), 7.66 (s, 1H), 7.64-7.47 (m, 2H), 7.42 (t, J=8.0 Hz, 1H), 7.31 (s, 1H), 7.16-7.06 (m, 2H), 7.06-6.93 (m, 2H), 6.74-6.71 (m, 2H), 6.47 (s, 1H), 6.02 (d, J=3.6 Hz, 1H), 4.77-4.69 (m, 1H), 4.35-4.29 (m, 1H), 3.94 (s, 3H), 3.92-3.90 (m, 1H), 3.87 (s, 3H), 3.78-3.57 (m, 3H), 3.49 (t, J=11.8 Hz, 2H), 3.27 (d, J=6.8 Hz, 2H), 3.24-3.20 (m, 3H), 3.16-3.10 (m, 1H), 2.26-2.18 (m, 1H), 2.03-1.96 (m, 1H) 1.87-1.81 (m, 2H), 1.76-1.70 (m, 4H), 1.68-1.66 (m, 1H) 1.60-1.42 (m, 10H), 1.40-1.38 (m, 4H), 1.35-1.31 (m, 10H), 1.28-1.25 (m, 4H).

### Example 105 Preparation of 4-{2-[(2S)-4-[(5,6-dimethoxypyridin-2-yl)methyl]-2-(2-isopropoxyphenyl)piperazin-1-yl]-7-azaspiro[3.5]nonan-7-yl}-2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-N-[3-nitro-4-({[(1r,4r)-4-hydroxy-4-methylcyclohexyl]methyl}amino)benzenesulfonyl]benzamide

Synthesis of 6-iodo-2,3-dimethoxypyridine: Into a 50 mL round-bottom flask, were placed 2-bromo-6-iodo-3-methoxypyridine (7.0 g, 22.3 mmol, 1.0 eq), sodium methoxide (1.8 g, 33.4 mmol, 1.5 eq), DMSO (35 mL). The resulting solution was stirred for 16 hours at 100° C. The reaction was then quenched by the addition of water (50 mL) and extracted with ethyl acetate (2×50 mL). The combined organic layer was washed with water (2×50 mL), brine (2×50 mL) and dried over anhydrous Na₂SO₄. After filtration, the filtrate was concentrated under vacuum to give 6-iodo-2,3-dimethoxypyridine as an off-white solid (5.0 g, 98.1%). LC-MS (ESI, m/z) M+1: 266. ¹H NMR (300 MHz, DMSO-d₆) δ 7.33 (d, J=8.1 Hz, 1H), 7.05 (d, J=8.1 Hz, 1H), 3.84 (s, 3H), 3.76 (s, 3H).

Synthesis of 5,6-dimethoxypyridine-2-carbaldehyde: Into a 250 mL 3-necked round-bottom flask, were placed 6-iodo-2,3-dimethoxypyridine (5.0 g, 18.9 mmol, 1.0 eq), THE (50 mL), which was followed by the addition of n-BuLi (1.8 g, 28.3 mmol, 1.5 eq) at −78° C. The resulting mixture was stirred for 30 minutes at −78° C. To the above mixture was added DMF (1.9 g, 26.4 mmol, 1.4 eq) at −78° C. The resulting mixture was stirred for additional 30 minutes at −78° C. The reaction was then quenched by the addition of water (50 mL) and extracted with ethyl acetate (2×50 mL). The combined organic layer was washed with brine (2×50 mL). The mixture was dried over anhydrous Na₂SO₄. After filtration, the filtrate was concentrated under vacuum. The crude residue was purified onto a silica gel column and eluted with ethyl acetate/petroleum ether=1:1 to give 5,6-dimethoxypyridine-2-carbaldehyde as a pink solid (3.0 g, 95.1%). LC-MS (ESI, m/z) M+1: 168. ¹H NMR (300 MHz, DMSO-d₆) δ 9.78 (s, 1H), 7.64 (d, J=8.1 Hz, 1H), 7.46 (d, J=8.1 Hz, 1H), 3.96 (s, 3H), 3.90 (s, 3H).

Synthesis of (2S)-1-benzyl-4-[(5,6-dimethoxypyridin-2-yl)methyl]-2-(2-isopropoxyphenyl)piperazine (assumed): Into a 40-mL sealed-tube, were placed (2S)-1-benzyl-2-(2-isopropoxyphenyl)piperazine (assumed) (150 mg, 0.5 mmol, 1.0 eq), 5,6-dimethoxypyridine-2-carbaldehyde (97 mg, 0.6 mmol, 1.2 eq), MeOH (3 mL), ZnCl₂ (198 mg, 1.4 mmol, 3.0 eq), NaBH₃CN (91 mg, 1.4 mmol, 3.0 eq). The resulting solution was stirred for 2 hours at 25° C. The reaction was then quenched by the addition of water (40 mL) and extracted with ethyl acetate (2×40 mL). The combined organic layer was washed with brine (2×40 mL) and dried over anhydrous Na₂SO₄. After filtration, the filtrate was concentrated under vacuum. The crude residue was purified onto a silica gel column and eluted with ethyl acetate/petroleum ether=1:2 to give (2S)-1-benzyl-4-[(5,6-dimethoxypyridin-2-yl)methyl]-2-(2-isopropoxyphenyl)piperazine (assumed) as a light yellow oil (80 mg, 35.9%). LC-MS (ESI, m/z) M+1: 462.

Synthesis of (3S)-1-[(5,6-dimethoxypyridin-2-yl)methyl]-3-(2-isopropoxyphenyl)piperazine (assumed): Into a 50-mL round-bottom flask, were placed (2S)-1-benzyl-4-[(5,6-dimethoxypyridin-2-yl)methyl]-2-(2-isopropoxyphenyl)piperazine (assumed) (70 mg, 0.2 mmol, 1.0 eq), THE (3 mL), Pd/C (8 mg, 0.1 mmol, 0.5 eq), Pd(OH)₂/C (11 mg, 0.1 mmol, 0.5 eq). The mixture was hydrogenated at 25° C. under H₂ (2 atm) for 16 hours. The resulting mixture was filtered. The filtrate was concentrated under vacuum to give (3S)-1-[(5,6-dimethoxypyridin-2-yl)methyl]-3-(2-isopropoxyphenyl)piperazine (assumed) as a light yellow oil (55 mg crude).

Synthesis of 4-{2-[(2S)-4-[(5,6-dimethoxypyridin-2-yl)methyl]-2-(2-isopropoxyphenyl)piperazin-1-yl]-7-azaspiro[3.5]nonan-7-yl}-N-[3-nitro-4-({[(1r,4r)-4-hydroxy-4-methylcyclohexyl]methyl}amino)benzenesulfonyl]-2-[(3R,8S)-15-{[2-(trimethylsilyl)ethoxy]methyl}-2,5-dioxa-9,15,17-triazatetracyclo[8.7.0.0\hat{ }{3,8}.0\hat{ }{12,16}]heptadeca-1(10),11,13,16-tetraen-9-yl]benzamide (assumed): Into an 8 mL vial were added N-[3-nitro-4-({[(1r,4r)-4-hydroxy-4-methylcyclohexyl]methyl}amino)benzenesulfonyl]-4-{2-oxo-7-azaspiro[3.5]nonan-7-yl}-2-[(3R,8S)-15-{[2-(trimethylsilyl)ethoxy]methyl}-2,5-dioxa-9,15,17-triazatetracyclo[8.7.0.0\hat{ }{3,8}.0\hat{ }{12,16}]heptadeca-1(10),11,13,16-tetraen-9-yl]benzamide (assumed) (87 mg, 0.1 mmol, 0.7 eq), (3S)-1-[(5,6-dimethoxypyridin-2-yl)methyl]-3-(2-isopropoxyphenyl)piperazine (45 mg, 0.1 mmol, 1.0 eq), NaBH₃CN (41 mg, 0.7 mmol, 5.0 eq), ZnCl₂ (90 mg, 0.7 mmol, 5 eq) and MeOH (2 mL). The resulting mixture was stirred for 2 hours at 70° C. The reaction was quenched by the addition of water (2 mL) and extracted with CH₂Cl₂ (3×3 mL). The combined organic layer was washed with brine (3 mL), dried over anhydrous Na₂SO₄. After filtration, the filtrate was concentrated under vacuum. The residue was purified by Prep-TLC (CH₂Cl₂/MeOH=20:1) to give 4-{2-[(2S)-4-[(5,6-dimethoxypyridin-2-yl)methyl]-2-(2-isopropoxyphenyl)piperazin-1-yl]-7-azaspiro[3.5]nonan-7-yl}-N-[3-nitro-4-({[(1r,4r)-4-hydroxy-4-methylcyclohexyl]methyl}amino)benzenesulfonyl]-2-[(3R,8S)-15-{[2-(trimethylsilyl)ethoxy]methyl}-2,5-dioxa-9,15,17-triazatetracyclo[8.7.0.0\hat{ }{3,8}.0\hat{ }{12,16}]heptadeca-1(10),11,13,16-tetraen-9-yl]benzamide (assumed) as a yellow solid (35 mg, 20.9%). LC-MS (ESI, m/z) M+1: 1300.

Synthesis of 4-{2-[(2S)-4-[(5,6-dimethoxypyridin-2-yl)methyl]-2-(2-isopropoxyphenyl)piperazin-1-yl]-7-azaspiro[3.5]nonan-7-yl}-2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-N-[3-nitro-4-({[(1r,4r)-4-hydroxy-4-methylcyclohexyl]methyl}amino)benzenesulfonyl]benzamide (assumed): Into an 8 mL vial were added 4-{2-[(2S)-4-[(5,6-dimethoxypyridin-2-yl)methyl]-2-(2-isopropoxyphenyl)piperazin-1-yl]-7-azaspiro[3.5]nonan-7-yl}-N-[3-nitro-4-({[(1r,4r)-4-hydroxy-4-methylcyclohexyl]methyl}amino)benzenesulfonyl]-2-[(3R,8S)-15-{[2-(trimethylsilyl)ethoxy]methyl}-2,5-dioxa-9,15,17-triazatetracyclo[8.7.0.0\hat{ }{3,8}.0\hat{ }{12,16}]heptadeca-1(10),11,13,16-tetraen-9-yl]benzamide (33 mg, 0.03 mmol, 1.0 eq), ethylenediamine (31 mg, 0.5 mmol, 20.0 eq) and TBAF in THF(1 M, 1 mL). The resulting mixture was stirred for 3 hours at 60° C. The reaction was quenched by the addition of water (3 mL) and extracted with CH₂Cl₂ (3×3 mL). The combined organic layer was washed with brine (5×3 mL), dried over anhydrous Na₂SO₄. After filtration, the filtrate was concentrated under vacuum. After filtration, the filtrate was concentrated under vacuum. The residue was purified by Prep-TLC (CH₂Cl₂/MeOH=20:1) to give 4-{2-[(2S)-4-[(5,6-dimethoxypyridin-2-yl)methyl]-2-(2-isopropoxyphenyl)piperazin-1-yl]-7-azaspiro[3.5]nonan-7-yl}-2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-N-[3-nitro-4-({[(1r,4r)-4-hydroxy- 4-methylcyclohexyl]methyl}amino)benzenesulfonyl]benzamide as a yellow solid (7.2 mg, 24.3%). LC-MS (ESI, m/z) M+1: 1169. ¹H NMR (300 MHz, Chloroform-d) δ 12.56 (s, 1H), 8.62 (d, J=2.1 Hz, 1H), 8.44 (s, 1H), 8.41 (s, 1H), 8.08 (d, J=9.3 Hz, 1H), 7.83-7.74 (m, 1H), 7.43 (s, 1H), 7.15 (s, 1H), 7.12-7.04 (m, 1H), 6.97 (d, J-7.8 Hz, 1H), 6.81 (d, J=9.0 Hz, 3H), 6.68 (d, J=9.3 Hz, 1H), 6.61 (s, 1H), 6.41 (s, 1H), 6.08 (d, J=3.5 Hz, 1H), 4.52 (d, J=5.4 Hz, 2H), 4.51-4.37 (m, 1H), 3.96 (s, 3H), 3.84 (s, 3H), 3.62 (s, 2H), 3.54-3.35 (m, 3H), 3.19 (t, J=6.0 Hz, 2H), 3.03 (s, 1H), 1.84 (d, J=13.5 Hz, 12H), 1.58-1.40 (m, 2H), 1.28 (d, J=13.2 Hz, 14H), 0.91-0.80 (m, 14H).

### Example 106 Preparation of 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-4-{2-[(2S)-2-(2-isopropoxyphenyl)-4-[(6-methoxy-5-methylpyridin-2-yl)methyl]piperazin-1-yl]-7-azaspiro[3.5]nonan-7-yl}-N-[3-nitro-4-({[(1r,4r)-4-hydroxy-4-methylcyclohexyl]methyl}amino)benzenesulfonyl]benzamide (assumed)

Synthesis of 6-methoxy-5-methylpyridine-2-carbaldehyde: A solution of 1-methyl-piperazine (6.0 g, 59.9 mmol, 1.6 eq) in THE was treated with n-BuLi in hexanes (22 mL, 233.5 mmol, 6.4 eq) for 30 min at −78° C. under nitrogen atmosphere, which was followed by the addition of 6-methoxypyridine-2-carbaldehyde (5.0 g, 36.5 mmol, 1.0 eq) dropwise at −78° C. The resulting mixture was stirred for 30 min at −78° C. under nitrogen atmosphere. To the above mixture was added t-BuLi (50 mL) dropwise over 20 min at −78° C. The resulting mixture was stirred for additional 3 hours at −78° C. The resulting mixture was allowed to warm to −40° C. for 15 min under nitrogen atmosphere. CH₃I (35.5 g, 250.1 mmol, 6.9 eq) was added slowly at −78° C., and the resulting mixture was allowed to 25° C. The resulting mixture was poured into a vigorously stirred cold brine (50 mL) and then extracted with ethyl ether (3×50 mL). The combined organic layer was dried over anhydrous Na₂SO₄, filtered and concentrated under vacuum. The crude residue was applied onto a silica gel column and eluted with ethyl acetate/petroleum ether=1:20 to give 6-methoxy-5-methylpyridine-2-carbaldehyde as a white solid (2.2 g, 39.9%). LC-MS (ESI, m/z) M+1: 152.

Synthesis of (2S)-1-benzyl-2-(2-isopropoxyphenyl)-4-[(6-methoxy-5-methylpyridin-2-yl)methyl]piperazine (assumed): A solution of (2S)-1-benzyl-2-(2-isopropoxyphenyl)piperazine (150 mg, 0.5 mmol, 1.0 eq) and 6-methoxy-5-methylpyridine-2-carbaldehyde (88 mg, 0.56 mmol, 1.2 eq) in MeOH (4 mL) was treated with ZnCl₂ (329 mg, 2.4 mmol, 5.0 eq) for 30 min at 25° C., which was followed by the addition of NaBH₃CN (152 mg, 2.4 mmol, 5.0 eq) dropwise at 25° C. The resulting mixture was stirred for 2 hours at 25° C. The reaction was quenched by the addition of water (2 mL) and then extracted with EtOAc (3×10 mL). The combined organic layer was washed with brine (10 mL), dried over anhydrous Na₂SO₄. After filtration, the filtrate was concentrated under vacuum. The crude residue was applied onto a silica gel column and eluted with THF/petroleum ether=1:20 to give (2S)-1-benzyl-2-(2-isopropoxyphenyl)-4-[(6-methoxy-5-methylpyridin-2-yl)methyl]piperazine (assumed) (100 mg, 46.4%) as a colorless oil. LC-MS (ESI, m/z) M+1: 446.

Synthesis of (3S)-3-(2-isopropoxyphenyl)-1-[(6-methoxy-5-methylpyridin-2-yl)methyl]piperazine (assumed): Into a 50 mL round-bottom flask were added (2S)-1-benzyl-2-(2-isopropoxyphenyl)-4-[(6-methoxy-5-methylpyridin-2-yl)methyl]piperazine (90 mg, 0.2 mmol, 1.0 eq), Pd(OH)₂/C (14 mg, 0.1 mmol, 0.5 eq), Pd/C (11 mg, 0.1 mmol, 0.5 eq) and THE (2 mL) at 0° C. The resulting mixture was stirred for 3 hours at 0° C. under H₂ (1 atm). The resulting mixture was filtered, the filter cake was washed with THE (3×5 mL). The filtrate was concentrated under vacuum. The crude product (3S)-3-(2-isopropoxyphenyl)-1-[(6-methoxy-5-methylpyridin-2-yl)methyl]piperazine (assumed) was used directly to the next step without further purification (65 mg, 90.5%).

Synthesis of 4-{2-[(2S)-2-(2-isopropoxyphenyl)-4-[(6-methoxy-5-methylpyridin-2-yl)methyl]piperazin-1-yl]-7-azaspiro[3.5]nonan-7-yl}-N-[3-nitro-4-({[(1r,4r)-4-hydroxy-4-methylcyclohexyl]methyl}amino)benzenesulfonyl]-2-[(3R,8S)-15-{[2-(trimethylsilyl)ethoxy]methyl}-2,5-dioxa-9,15,17-triazatetracyclo[8.7.0.0\hat{ }{3,8}.0\hat{ }{12,16}]heptadeca-1(10),11,13,16-tetraen-9-yl]benzamide (assumed): Into an 8 mL vial were added N-[3-nitro-4-({[(1r,4r)-4-hydroxy-4-methylcyclohexyl]methyl}amino)benzenesulfonyl]-4-{2-oxo-7-azaspiro[3.5]nonan-7-yl}-2-[(3R,8S)-15-{[2-(trimethylsilyl)ethoxy]methyl}-2,5-dioxa-9,15,17-triazatetracyclo[8.7.0.0\hat{ }{3,8}.0\hat{ }{12,16}]heptadeca-1(10),11,13,16-tetraen-9-yl]benzamide (112 mg, 0.1 mmol, 0.7 eq), NaBH₃CN (53 mg, 0.8 mmol, 5.0 eq), ZnCl₂ (115 mg, 0.8 mmol, 5.0 eq) and MeOH (1 mL). The resulting mixture was stirred for 2 hours 25° C. The reaction was quenched by the addition of water (2 mL) and then extracted with CH₂Cl₂ (3×3 mL). The combined organic layer was washed with brine (3 mL), dried over anhydrous Na₂SO₄. After filtration, the filtrate was concentrated under vacuum. The residue was purified by Prep-TLC (CH₂Cl₂/MeOH=20:1) to give 4-{2-[(2S)-2-(2-isopropoxyphenyl)-4-[(6-methoxy-5-methylpyridin-2-yl)methyl]piperazin-1-yl]-7-azaspiro[3.5]nonan-7-yl}-N-[3-nitro-4-({[(1r,4r)-4-hydroxy-4-methylcyclohexyl]methyl}amino)benzenesulfonyl]-2-[(3R,8S)-15-{[2-(trimethylsilyl)ethoxy]methyl}-2,5-dioxa-9,15,17-triazatetracyclo[8.7.0.0\hat{ }{3,8}.0\hat{ }{12,16}]heptadeca-1(10),11,13,16-tetraen-9-yl]benzamide (assumed) as a yellow solid (50 mg, 23.1%). LC-MS (ESI, m/z) M+1: 1284.

Synthesis of 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-4-{2-[(2S)-2-(2-isopropoxyphenyl)-4-[(6-methoxy-5-methylpyridin-2-yl)methyl]piperazin-1-yl]-7-azaspiro[3.5]nonan-7-yl}-N-[3-nitro-4-({[(1r,4r)-4-hydroxy-4-methylcyclohexyl]methyl}amino)benzenesulfonyl]benzamide (assumed): Into an 8 mL vial were added 4-{2-[(2S)-2-(2-isopropoxyphenyl)-4-[(6-methoxy-5-methylpyridin-2-yl)methyl]piperazin-1-yl]-7-azaspiro[3.5]nonan-7-yl}-N-[3-nitro-4-({[(1r,4r)-4-hydroxy-4-methylcyclohexyl]methyl}amino)benzenesulfonyl]-2-[(3R,8S)-15-{[2-(trimethylsilyl)ethoxy]methyl}-2,5-dioxa-9,15,17-triazatetracyclo[8.7.0.0\hat{ }{3,8}.0\hat{ }{12,16}]heptadeca-1(10),11,13,16-tetraen-9-yl]benzamide (48 mg, 0.04 mmol, 1.0 eq), ethylenediamine (45 mg, 0.7 mmol, 20.0 eq) and TBAF in THF(1 M, 2 mL). The resulting mixture was stirred for 3 hours at 70° C. The reaction was quenched by the addition of water (2 mL) and then extracted with CH₂Cl₂ (3×5 mL). The combined organic layer was washed with aq. HCl (1 M, 3×3 mL), and dried over anhydrous Na₂SO₄. After filtration, the filtrate was concentrated under vacuum. The residue was purified by Prep-TLC (CH₂Cl₂/MeOH=25:1) to give 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-4-{2-[(2S)-2-(2-isopropoxyphenyl)-4-[(6-methoxy-5-methylpyridin-2-yl)methyl]piperazin-1-yl]-7-azaspiro[3.5]nonan-7-yl}-N-[3-nitro-4-({[(1r,4r)-4-hydroxy-4-methylcyclohexyl]methyl}amino)benzenesulfonyl]benzamide (assumed) (10 mg, 23.19%) as a yellow solid. LC-MS (ESI, m/z) M+1: 1154. ¹H NMR (300 MHz, Methanol-d₄) δ 8.49 (dd, J=6.2, 2.3 Hz, 1H), 7.86 (d, J=9.3 Hz, 1H), 7.66-7.46 (m, 3H), 7.42 (d, J=7.1 Hz, 1H), 7.24 (t, J=7.5 Hz, 1H), 7.12-6.81 (m, 4H), 6.67 (d, J=9.4 Hz, 2H), 6.51 (d, J=13.8 Hz, 1H), 5.99 (dd, J=17.7, 3.4 Hz, 1H), 4.71-4.54 (m, 1H), 4.36-4.09 (m, 3H), 3.93 (d, J=3.4 Hz, 5H), 3.70 (d, J=14.3 Hz, 4H), 3.54 (d, J=34.3 Hz, 3H), 2.94-2.74 (m, 2H), 2.56 (d, J=12.5 Hz, 3H), 2.37-2.18 (m, 2H), 2.15 (s, 3H), 2.06-1.91 (m, 2H), 1.84 (d, J=13.4 Hz, 5H), 1.73 (d, J=12.2 Hz, 6H), 1.53 (t, J=12.6 Hz, 5H), 1.44-1.36 (m, 3H), 1.33 (d, J=2.1 Hz, 3H), 1.31 (d, J=2.6 Hz, 3H), 1.26 (d, J=6.8 Hz, 2H), 0.91 (d, J=6.9 Hz, 2H).

### Example 108 Preparation of 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-4-{2-[(2S)-2-(2-isopropoxyphenyl)-4-[(1r,4r)-4-hydroxy-4-methylcyclohexyl]piperazin-1-yl]-7-azaspiro[3.5]nonan-7-yl}-N-[3-nitro-4-({[(1r,4r)-4-hydroxy-4-methylcyclohexyl]methyl}amino)benzenesulfonyl]benzamide (assumed)

Synthesis of (1r,4r)-4-[(3S)-3-(2-isopropoxyphenyl)piperazin-1-yl]-1-methylcyclohexan-1-ol (assumed) and (1s,4s)-4-[(3S)-3-(2-isopropoxyphenyl)piperazin-1-yl]-1-methylcyclohexan-1-ol (assumed): Into a 40 mL vial were added 4-hydroxy-4-methylcyclohexan-1-one (200 mg, 1.6 mmol, 1.0 eq), (2S)-2-(2-isopropoxyphenyl)piperazine hydrochloride (412 mg, 1.9 mmol, 1.2 eq), NaBH₃CN (490 mg, 7.8 mmol, 5.0 eq), ZnCl₂ (1.1 g, 7.8 mmol, 5.0 eq) and MeOH (5 mL). The resulting mixture was stirred for 6 hours. The crude residue was purified by reverse flash chromatography using the following conditions: column, C18 silica gel; mobile phase, MeCN in water (0.1% NH₃—H₂O), 10% to 50% gradient in 10 min; detector, UV 254 nm. Finally, (1r,4r)-4-[(3S)-3-(2-isopropoxyphenyl)piperazin-1-yl]-1-methylcyclohexan-1-ol (assumed) (65 mg, 12.5%) and (1s,4s)-4-[(3S)-3-(2-isopropoxyphenyl)piperazin-1-yl]-1-methylcyclohexan-1-ol (assumed) was obtained as an off-white solid (60 mg, 11.6%). LC-MS (ESI, m/z) M+1: 333.

Synthesis of 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-4-{2-[(2S)-2-(2-isopropoxyphenyl)-4-[(1r,4r)-4-hydroxy-4-methylcyclohexyl]piperazin-1-yl]-7-azaspiro[3.5]nonan-7-yl}-N-[3-nitro-4-({[(1r,4r)-4-hydroxy-4-methylcyclohexyl]methyl}amino)benzenesulfonyl]benzamide (assumed): Into an 8 mL vial were added (1r,4r)-4-[(3S)-3-(2-isopropoxyphenyl)piperazin-1-yl]-1-methylcyclohexan-1-ol (40 mg, 0.1 mmol, 1.0 eq), 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-N-[3-nitro-4-({[(1r,4r)-4-hydroxy-4-methylcyclohexyl]methyl}amino)benzenesulfonyl]-4-{2-oxo-7-azaspiro[3.5]nonan-7-yl}benzamide (90 mg, 0.1 mmol, 0.9 eq), NaBH₃CN (38 mg, 0.6 mmol, 5 eq), ZnCl₂ (82 mg, 0.6 mmol, 5.0 eq) and MeOH (2 mL). The resulting mixture was stirred for 2 hours at 70° C. The reaction was quenched by the addition of water (2 mL) and then extracted with CH₂Cl₂ (3×5 mL). The combined organic layer was washed with brine (5 mL), dried over anhydrous Na₂SO₄. After filtration, the filtrate was concentrated under vacuum. The crude residue was purified by Prep-TLC (CH₂Cl₂/MeOH=20:1) to give 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-4-{2-[(2S)-2-(2-isopropoxyphenyl)-4-[(1r,4r)-4-hydroxy-4-methylcyclohexyl]piperazin-1-yl]-7-azaspiro[3.5]nonan-7-yl}-N-[3-nitro-4-({[(1r,4r)-4-hydroxy-4-methylcyclohexyl]methyl}amino)benzenesulfonyl]benzamide (assumed) as a yellow solid (28 mg, 20.6%). LC-MS (ESI, m/z) M+1: 1131. ¹H NMR (300 MHz, Methanol-d₄) δ 8.54 (d, J=2.2 Hz, 1H), 7.81 (d, J=9.0 Hz, 1H), 7.53-7.32 (m, 2H), 7.32-7.19 (m, 1H), 7.06 (d, J=3.5 Hz, 1H), 6.99 (t, J=9.7 Hz, 1H), 6.95-6.85 (m, 2H), 6.71-6.60 (m, 2H), 6.54 (s, 1H), 6.01 (d, J=3.4 Hz, 1H), 4.67 (dq, J=12.0, 5.8 Hz, 1H), 4.34-4.07 (m, 3H), 4.07-3.75 (m, 2H), 3.65 (s, 1H), 3.46 (d, J=11.9 Hz, 1H), 3.30-3.21 (m, 3H), 3.09 (d, J=18.2 Hz, 6H), 2.99-2.85 (m, 1H), 2.82-2.32 (m, 5H), 2.19 (d, J=17.9 Hz, 1H), 2.08-1.79 (m, 6H), 1.79-1.62 (m, 7H), 1.62-1.41 (m, 3H), 1.40-1.35 (m, 4H), 1.35-1.28 (m, 8H), 1.25 (d, J=2.2 Hz, 3H), 1.21 (d, J=2.1 Hz, 4H), 0.91 (dt, J=14.4, 7.9 Hz, 2H).

### Example 109 Preparation of 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-4-{2-[(2S)-2-(2-isopropoxyphenyl)-4-[(1s,4s)-4-hydroxy-4-methylcyclohexyl]piperazin-1-yl]-7-azaspiro[3.5]nonan-7-yl}-N-[3-nitro-4-({[(1r,4r)-4-hydroxy-4-methylcyclohexyl]methyl}amino)benzenesulfonyl]benzamide (assumed)

Synthesis of 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-4-{2-[(2S)-2-(2-isopropoxyphenyl)-4-[(1s,4s)-4-hydroxy-4-methylcyclohexyl]piperazin-1-yl]-7-azaspiro[3.5]nonan-7-yl}-N-[3-nitro-4-({[(1r,4r)-4-hydroxy-4-methylcyclohexyl]methyl}amino)benzenesulfonyl]benzamide (assumed): Into an 8 mL vial were added (1s,4s)-4-[(3S)-3-(2-isopropoxyphenyl)piperazin-1-yl]-1-methylcyclohexan-1-ol (40 mg, 0.1 mmol, 1.0 eq), 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-N-[3-nitro-4-({[(1r,4r)-4-hydroxy-4-methylcyclohexyl]methyl}amino)benzenesulfonyl]-4-{2-oxo-7-azaspiro[3.5]nonan-7-yl}benzamide (90 mg, 0.1 mmol, 0.9 eq), NaBH₃CN (38 mg, 0.6 mmol, 5.0 eq), ZnCl₂ (82 mg, 0.6 mmol, 5.0 eq) and MeOH (2 mL). The resulting mixture was stirred for 2 hours at 70° C. The reaction was quenched by the addition of water (2 mL) and then extracted with CH₂Cl₂ (3×5 mL). The combined organic layer was washed with brine (5 mL), dried over anhydrous Na₂SO₄. After filtration, the filtrate was concentrated under vacuum. The crude residue was purified by Prep-TLC (CH₂Cl₂/MeOH=10:1) to give 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-4-{2-[(2S)-2-(2-isopropoxyphenyl)-4-[(1s,4s)-4-hydroxy-4-methylcyclohexyl]piperazin-1-yl]-7-azaspiro[3.5]nonan-7-yl}-N-[3-nitro-4-({[(1r,4r)-4-hydroxy-4-methylcyclohexyl]methyl}amino)benzenesulfonyl]benzamide (assumed) as a yellow solid (32 mg, 23.5%). LC-MS (ESI, m/z) M+1: 1131. ¹H NMR (300 MHz, Methanol-d₄) δ 8.51 (dd, J=10.3, 2.2 Hz, 1H), 7.84 (d, J=9.0 Hz, 1H), 7.65 (d, J=8.7 Hz, 1H), 7.55-7.40 (m, 2H), 7.27 (t, J=8.3 Hz, 1H), 7.07 (d, J=3.5 Hz, 1H), 7.01 (s, 1H), 6.97 (d, J=6.0 Hz, 1H), 6.96-6.88 (m, 1H), 6.71-6.62 (m, 1H), 6.53 (d, J=15.2 Hz, 1H), 5.99 (dd, J=20.1, 3.5 Hz, 1H), 4.74-4.62 (m, 1H), 4.60 (s, 1H), 4.34-4.09 (m, 5H), 3.91 (d, J=7.5 Hz, 4H), 3.64 (d, J=12.4 Hz, 3H), 3.47 (d, J=6.5 Hz, 5H), 3.26 (d, J=6.8 Hz, 2H), 3.02 (s, 4H), 2.87 (s, 2H), 2.58-2.29 (m, 2H), 1.74 (d, J=12.3 Hz, 9H), 1.61-1.40 (m, 8H), 1.40-1.29 (m, 7H), 1.26 (d, J=3.6 Hz, 3H), 1.20 (d, J=2.6 Hz, 3H), 0.96 (d, J=6.9 Hz, 2H).

### Example 110 Preparation of 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-4-{2-[(2S)-2-(2-isopropoxyphenyl)-4-({8-methoxy-4,4-dimethyl-2H,3H-pyrano[2,3-c]pyridin-6-yl}methyl)piperazin-1-yl]-7-azaspiro[3.5]nonan-7-yl}-N-[3-nitro-4-({[(1r,4r)-4-hydroxy-4-methylcyclohexyl]methyl}amino)benzenesulfonyl]benzamide hydrochloride (assumed)

Synthesis of 2-chloro-6-iodo-3-[(3-methylbut-3-en-1-yl)oxy]pyridine: Into a 500-mL 3-necked round-bottom flask, were placed 2-chloro-6-iodopyridin-3-ol (20.0 g, 78.3 mmol, 1.0 eq), 3-methyl-3-buten-1-ol (6.7 g, 78.3 mmol, 1.0 eq), PPh₃ (22.6 g, 86.1 mmol, 1.1 eq), THE (200 mL). After that, DIAD (17.4 g, 86.1 mmol, 1.1 eq) was added at 0° C. under N₂ atmosphere. The mixture was stirred at 25° C. for 2 hours under N₂ atmosphere. The reaction was then quenched by the addition of water (300 mL) and then extracted with ethyl acetate (2×300 mL). The combined organic layer was washed with brine (2×300 mL) and dried over anhydrous Na₂SO₄. After filtration, the filtrate was concentrated under vacuum. The crude residue was purified onto a silica gel column and eluted with ethyl acetate/petroleum ether=1:2 to give 2-chloro-6-iodo-3-[(3-methylbut-3-en-1-yl)oxy]pyridine as a light yellow oil (17.0 g, 67.1%). ¹H NMR (400 MHz, DMSO-d₆) δ 7.78 (d, J=8.4 Hz, 1H), 7.40 (d, J=8.4 Hz, 1H), 4.82-479 (m, 2H), 4.21 (t, J=6.8 Hz, 2H), 2.50-2.42 (m, 2H), 1.77 (t, J=1.2 Hz, 3H).

Synthesis of 6-iodo-2-methoxy-3-[(3-methylbut-3-en-1-yl)oxy]pyridine: Into a 500-mL round-bottom flask, were placed 2-chloro-6-iodo-3-[(3-methylbut-3-en-1-yl)oxy]pyridine (17.0 g, 52.5 mmol, 1.0 eq), MeONa (4.3 g, 78.8 mmol, 1.5 eq), DMSO (170 mL). The resulting solution was stirred for 2 hours at 100° C. The reaction was then quenched by the addition of water (300 mL) and then extracted with ethyl acetate (2×300 mL). The combined organic layer was washed with brine (3×300 mL) and dried over anhydrous Na₂SO₄. After filtration, the filtrate was concentrated under vacuum. The crude residue was purified onto a silica gel column and eluted with ethyl acetate/petroleum ether=1:8 to give 6-iodo-2-methoxy-3-[(3-methylbut-3-en-1-yl)oxy]pyridine as a light yellow oil (3.8 g, 22.7%). ¹H NMR (400 MHz, DMSO-d₆) δ 7.32 (d, J=8.0 Hz, 1H), 7.11 (d, J=8.2 Hz, 1H), 4.83-4.75 (m, 2H), 4.07 (t, J=7.0 Hz, 2H), 3.83 (s, 3H), 2.43 (t, J=6.8 Hz, 2H), 1.76 (t, J=1.2 Hz, 3H).

Synthesis of 6-iodo-8-methoxy-4,4-dimethyl-2H,3H-pyrano[2,3-c]pyridine: Into a 100-mL round-bottom flask, were placed 6-iodo-2-methoxy-3-[(3-methylbut-3-en-1-yl)oxy]pyridine (3.5 g, 11.0 mmol, 1.0 eq), EtOH (40 mL), TFA (2.5 g, 21.9 mmol, 2.0 eq), Fe(acac)₃ (1.9 g, 5.5 mmol, 0.5 eq), phenylsilane (3.0 g, 27.4 mmol, 2.5 eq), 2-(tert-butylperoxy)-2-methylpropane (4.8 g, 32.9 mmol, 3.0 eq). The resulting solution was stirred for 3 hours at 60° C. The reaction was then quenched by the addition of water (100 mL) and extracted with ethyl acetate (2×150 mL). The combined organic layer was washed with brine (3×100 mL) and dried over anhydrous Na₂SO₄. After filtration, the filtrate was concentrated under vacuum. The crude residue was purified onto a silica gel column and eluted with ethyl acetate/petroleum ether=1:10 to give 6-iodo-8-methoxy-4,4-dimethyl-2H,3H-pyrano[2,3-c]pyridine as a light yellow oil (300 mg, 8.6%). ¹H NMR (400 MHz, DMSO-d₆) δ 7.33 (s, 1H), 4.20-4.13 (m, 2H), 3.80 (s, 3H), 1.86-1.73 (m, 2H), 1.26 (s, 6H).

Synthesis of 8-methoxy-4,4-dimethyl-2H,3H-pyrano[2,3-c]pyridine-6-carbaldehyde: Into a 50-mL 3-necked round-bottom flask, were placed 6-iodo-8-methoxy-4,4-dimethyl-2H,3H-pyrano[2,3-c]pyridine (270 mg, 0.846 mmol, 1.0 eq), THE (4 mL). After that, n-BuLi (2.5 M in hexanes, 0.4 mL) was added at −78° C. Then the mixture was stirred at −78° C. for 30 minutes. After that, DMF (87 mg, 1.2 mmol, 1.4 eq) was added dropwise at −78° C. The mixture was stirred at −78° C. for 2 hours. The reaction was then quenched by the addition of aq. NH₄Cl (10 mL) and extracted with ethyl acetate (2×30 mL). The combined organic layer was washed with brine (2×30 mL). The combined organic phase was dried over anhydrous Na₂SO₄. After filtration, the filtrate was concentrated under vacuum. The crude residue was purified onto a silica gel column and eluted with ethyl acetate/petroleum ether=1:5 to give 8-methoxy-4,4-dimethyl-2H,3H-pyrano[2,3-c]pyridine-6-carbaldehyde as a light yellow oil (110 mg, 58.8%). ¹H NMR (300 MHz, Chloroform-d) δ 9.85 (s, 1H), 7.62 (s, 1H), 4.44-4.35 (m, 2H), 4.11 (s, 3H), 1.94-1.89 (m, 3H), 1.38 (d, J=2.1 Hz, 6H).

Synthesis of (3S)-3-(2-isopropoxyphenyl)-1-({8-methoxy-4,4-dimethyl-2H,3H-pyrano[2,3-c]pyridin-6-yl}methyl)piperazine (assumed): Into a 20-mL sealed-tube, were placed (2S)-2-(2-isopropoxyphenyl)piperazine (assumed) (110 mg, 0.5 mmol, 1.0 eq), 8-methoxy-4,4-dimethyl-2H,3H-pyrano[2,3-c]pyridine-6-carbaldehyde (110 mg, 0.5 mmol, 1.0 eq), DCE (3 mL), NaBH(OAc)₃ (317 mg, 1.5 mmol, 3.0 eq). The resulting solution was stirred for 2 hours at 25° C. The reaction was then quenched by the addition of water (20 mL) and extracted with dichloromethane/methanol=20:1 (2×20 mL). The combined organic layer was washed with brine (2×40 mL) and dried over anhydrous Na₂SO₄. After filtration, the filtrate was concentrated under vacuum. The crude residue was purified onto a silica gel column and eluted with dichloromethane/methanol=15:1 to give (3S)-3-(2-isopropoxyphenyl)-1-({8-methoxy-4,4-dimethyl-2H,3H-pyrano[2,3-c]pyridin-6-yl}methyl)piperazine (assumed) as an off-white solid (90 mg, 42.4%). LC-MS (ESI, m/z) M+1: 426.

Synthesis of 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-4-{2-[(2S)-2-(2-isopropoxyphenyl)-4-({8-methoxy-4,4-dimethyl-2H,3H-pyrano[2,3-c]pyridin-6-yl}methyl)piperazin-1-yl]-7-azaspiro[3.5]nonan-7-yl}-N-[3-nitro-4-({[(1r,4r)-4-hydroxy-4-methylcyclohexyl]methyl}amino)benzenesulfonyl]benzamide hydrochloride (assumed): Into an 8-mL sealed-tube, were placed (3S)-3-(2-isopropoxyphenyl)-1-({8-methoxy-4,4-dimethyl-2H,3H-pyrano[2,3-c]pyridin-6-yl}methyl)piperazine (assumed) (40 mg, 0.1 mmol, 1.0 eq), 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-N-[3-nitro-4-({[(1r,4r)-4-hydroxy- 4-methylcyclohexyl]methyl}amino)benzenesulfonyl]-4-{2-oxo-7-azaspiro[3.5]nonan-7-yl}benzamide (77 mg, 0.1 mmol, 1.0 eq), MeOH (1 mL), ZnCl₂ (384 mg, 2.8 mmol, 3.0 eq), NaBH₃CN (18 mg, 0.3 mmol, 3.0 eq). The resulting solution was stirred for 3 hours at 70° C. The reaction was then quenched by the addition of water (20 mL) and extracted with dichloromethane/methanol=10:1 (2×20 mL). The combined organic layer was washed with brine (2×20 mL) and the organic layers combined. The mixture was dried over anhydrous Na₂SO₄. After filtration, the filtrate was concentrated under vacuum. The crude product was purified by Prep-HPLC using the following conditions (Prep-HPLC-006): Column, YMC-Actus Triart C18 ExRS, 30*150 mm, 5 μm; mobile phase, water (0.05% HCl) and CH₃CN (30% CH₃CN up to 45% in 7 min). Finally, 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-4-{2-[(2S)-2-(2-isopropoxyphenyl)-4-({8-methoxy-4,4-dimethyl-2H,3H-pyrano[2,3-c]pyridin-6-yl}methyl)piperazin-1-yl]-7-azaspiro[3.5]nonan-7-yl}-N-[3-nitro-4-({[(1r,4r)-4-hydroxy-4-methylcyclohexyl]methyl}amino)benzenesulfonyl]benzamide hydrochloride (assumed) was obtained as a light yellow solid (15 mg, 12.7%). LC-MS (ESI, m/z) M+1: 1224. ¹H NMR (400 MHz, Methanol-d₄) δ 8.52 (dd, J=17.4, 2.4 Hz, 1H), 7.84-7.74 (m, 3H), 7.69-7.60 (m, 1H), 7.49 (ddd, J=8.8, 7.6, 1.6 Hz, 1H), 7.20-7.13 (m, 3H), 7.11-7.06 (m, 2H), 6.96-6.78 (m, 2H), 6.70 (s, 1H), 5.47 (br, 1H), 4.84-4.77 (m, 1H), 4.52-4.41 (m, 2H), 4.32-4.17 (m, 4H), 4.12 (t, J=12.8 Hz, 1H), 3.99 (d, J=3.8 Hz, 3H), 3.97-3.82 (m, 6H), 3.76-3.44 (m, 4H), 3.37-3.15 (m, 7H), 2.33-2.22 (m, 2H), 1.91-1.83 (m, 4H), 1.75-1.70 (m, 3H), 1.66-1.60 (m, 2H), 1.56-1.45 (m, 6H), 1.43 (d, J=6.0 Hz, 6H), 1.36-1.35 (m, 7H), 1.28-1.26 (m, 5H).

### Example 111 Preparation of 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-4-{2-[(2S)-2-(2-isopropoxyphenyl)-4-({5-methoxy-6-[(3R)-3-methylmorpholin-4-yl]pyridin-3-yl}methyl)piperazin-1-yl]-7-azaspiro[3.5]nonan-7-yl}-N-[3-nitro-4-({[(1r,4r)-4-hydroxy-4-methylcyclohexyl]methyl}amino)benzenesulfonyl]benzamide (assumed)

Synthesis of (3R)-4-(3-methoxypyridin-2-yl)-3-methylmorpholine: Into a 500 mL round-bottom flask were added 2-bromo-3-methoxypyridine (10.0 g, 53.2 mmol, 1.0 eq), (3R)-3-methylmorpholine (8.1 g, 80.0 mmol, 1.5 eq), Cs₂CO₃ (10.1 g, 132.9 mmol, 2.5 eq) and DMSO (120 mL). The resulting mixture was stirred for 36 hours at 140° C. The reaction was quenched by the addition of water (50 mL) and extracted with EtOAc (3×200 mL). The combined organic layer was washed with brine (100 mL), dried over anhydrous Na₂SO₄. After filtration, the filtrate was concentrated under vacuum. The crude residue was applied onto a silica gel column and eluted with ethyl acetate/petroleum ether=1:5 to give (3R)-4-(3-methoxypyridin-2-yl)-3-methylmorpholine as an orange solid (4.1 g, 37.0%). LC-MS (ESI, m/z) M+1: 209.

Synthesis of (3R)-4-(5-bromo-3-methoxypyridin-2-yl)-3-methylmorpholine: Into a 50 mL round-bottom flask were added (3R)-4-(3-methoxypyridin-2-yl)-3-methylmorpholine (4.0 g, 19.2 mmol, 1.0 eq), NBS (6.8 g, 38.4 mmol, 2.0 eq) and DMF (10 mL). The resulting mixture was stirred for 4 hours at 80° C. The reaction was quenched by the addition of water (5 mL) and then extracted with EtOAc (3×20 mL). The combined organic layer was washed with brine (20 mL), dried over anhydrous Na₂SO₄. After filtration, the filtrate was concentrated under vacuum. The crude residue was applied onto a silica gel column and eluted with ethyl acetate/petroleum ether=1:5 to give (3R)-4-(5-bromo-3-methoxypyridin-2-yl)-3-methylmorpholine as a red solid (1.8 g, 32.6%). ¹H NMR (300 MHz, Chloroform-d) δ 7.94 (d, J=2.0 Hz, 1H), 7.15 (d, J=2.0 Hz, 1H), 4.15 (qt, J=7.4, 3.6 Hz, 1H), 3.93 (dt, J=10.0, 3.7 Hz, 2H), 3.86 (s, 3H), 3.77 (ddd, J=11.3, 9.9, 3.0 Hz, 1H), 3.64 (dd, J=11.3, 3.3 Hz, 1H), 3.48 (ddd, J=13.2, 9.8, 3.4 Hz, 1H), 3.29 (dt, J=13.0, 3.2 Hz, 1H), 1.15 (d, J=6.6 Hz, 3H).

Synthesis of 5-methoxy-6-[(3R)-3-methylmorpholin-4-yl]pyridine-3-carbaldehyde: A solution of (3R)-4-(5-bromo-3-methoxypyridin-2-yl)-3-methylmorpholine (500 mg, 1.7 mmol, 1.0 eq) in THE (8 mL) was treated with n-BuLi in hexanes (133.9 mg, 2.1 mmol, 1.2 eq) for 1 hour at −78° C., which was followed by the addition of DMF (763 mg, 10.4 mmol, 6.0 eq) dropwise at −78° C. under nitrogen atmosphere. The resulting mixture was allowed to 25° C. The reaction was quenched by the addition of sat. NH₄Cl (10 mL) at 0° C., and extracted with EtOAc (3×10 mL). The combined organic layer was washed with brine (10 mL), dried over anhydrous Na₂SO₄. After filtration, the filtrate was concentrated under vacuum. The crude residue was applied onto a silica gel column and eluted with ethyl acetate/petroleum ether=1:5 to give 5-methoxy-6-[(3R)-3-methylmorpholin-4-yl]pyridine-3-carbaldehyde as an orange solid (180 mg, 43.8%). ¹H NMR (300 MHz, Chloroform-d) δ 9.85 (s, 1H), 8.24 (d, J=1.8 Hz, 1H), 7.42 (d, J=1.8 Hz, 1H), 4.68 (d, J=7.5 Hz, 1H), 4.12 (d, J=13.4 Hz, 1H), 3.97 (d, J=12.1 Hz, 1H), 3.89 (s, 3H), 3.83 (dd, J=11.5, 3.1 Hz, 1H), 3.79-3.65 (m, 2H), 3.53 (ddd, J=13.4, 11.8, 3.4 Hz, 1H), 1.35 (d, J=6.8 Hz, 3H).

Synthesis of (3R)-4-(5-{[(3S)-3-(2-isopropoxyphenyl)piperazin-1-yl]methyl}-3-methoxypyridin-2-yl)-3-methylmorpholine (assumed): Into an 8 mL vial were added 5-methoxy-6-[(3R)-3-methylmorpholin-4-yl]pyridine-3-carbaldehyde (60 mg, 0.3 mmol, 1.0 eq), (2S)-2-(2-isopropoxyphenyl)piperazine (56 mg, 0.2 mmol, 1.0 eq), NaBH₃CN (80 mg, 1.3 mmol, 5.0 eq), ZnCl₂ (173 mg, 1.3 mmol, 5.0 eq) and MeOH (2 mL). The resulting mixture was stirred for 4 hours at 25° C. The reaction was quenched by the addition of water (2 mL) and then extracted with CH₂Cl₂ (3×5 mL). The combined organic layer was washed with brine (5 mL), dried over anhydrous Na₂SO₄. After filtration, the filtrate was concentrated under vacuum. The crude residue was purified by Prep-TLC (CH₂Cl₂/MeOH=10:1) to give (3R)-4-(5-{[(3S)-3-(2-isopropoxyphenyl)piperazin-1-yl]methyl}-3-methoxypyridin-2-yl)-3-methylmorpholine (assumed) as an off-white solid (62 mg, 55.4%). ¹H NMR (300 MHz, Chloroform-d) δ 7.77 (d, J=1.8 Hz, 1H), 7.44 (dd, J=7.6, 1.8 Hz, 1H), 7.21 (td, J=7.8, 1.8 Hz, 1H), 7.11 (d, J=1.9 Hz, 1H), 6.99-6.78 (m, 2H), 4.62-4.53 (m, 1H), 4.26 (dd, J=10.1, 2.8 Hz, 1H), 4.12 (qd, J=6.7, 3.4 Hz, 1H), 3.77 (ddd, J=11.3, 9.4, 2.9 Hz, 2H), 3.69-3.53 (m, 2H), 3.56-3.34 (m, 3H), 3.19 (dd, J=11.9, 3.1 Hz, 3H), 3.07-2.77 (m, 5H), 2.44-2.11 (m, 2H), 1.34 (d, J=6.1 Hz, 3H), 1.29-1.22 (m, 3H), 1.12 (d, J=6.6 Hz, 3H).

Synthesis of 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-4-{2-[(2S)-2-(2-isopropoxyphenyl)-4-({5-methoxy-6-[(3R)-3-methylmorpholin-4-yl]pyridin-3-yl}methyl)piperazin-1-yl]-7-azaspiro[3.5]nonan-7-yl}-N-[3-nitro-4-({[(1r,4r)-4-hydroxy-4-methylcyclohexyl]methyl}amino)benzenesulfonyl]benzamide (assumed): Into an 8 mL vial were added (3R)-4-(5-{[(3S)-3-(2-isopropoxyphenyl)piperazin-1-yl]methyl}-3-methoxypyridin-2-yl)-3-methylmorpholine (40 mg, 0.1 mmol, 1.0 eq) and 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-N-[3-nitro-4-({[(1r,4r)-4-hydroxy-4-methylcyclohexyl]methyl}amino)benzenesulfonyl]-4-{2-oxo-7-azaspiro[3.5]nonan-7-yl}benzamide (80 mg, 0.1 mmol, 1.1 eq), NaBH₃CN (29 mg, 0.5 mmol, 5.0 eq), ZnCl₂ (62 mg, 0.5 mmol, 5.0 eq) and MeOH (2 mL). The resulting mixture was stirred for 4 hours at 70° C. The reaction was quenched by the addition of water (2 mL) and then extracted with CH₂Cl₂ (3×5 mL). The combined organic layer was washed with brine (3 mL), dried over anhydrous Na₂SO₄. After filtration, the filtrate was concentrated under vacuum. The residue was purified by Prep-TLC (CH₂Cl₂/MeOH 15:1) to give 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-4-{2-[(2S)-2-(2-isopropoxyphenyl)-4-({5-methoxy-6-[(3R)-3-methylmorpholin-4-yl]pyridin-3-yl}methyl)piperazin-1-yl]-7-azaspiro[3.5]nonan-7-yl}-N-[3-nitro-4-({[(1r,4r)-4-hydroxy-4-methylcyclohexyl]methyl}amino)benzenesulfonyl]benzamide (assumed) (25 mg, 22.2%) as a yellow solid. LC-MS (ESI, m/z) M+1: 1239. ¹H NMR (300 MHz, Chloroform-d) δ 12.58 (s, 1H), 8.65 (s, 1H), 8.57-8.35 (m, 2H), 8.28-7.99 (m, 2H), 7.99-7.65 (m, 2H), 7.44 (d, J=7.5 Hz, 1H), 7.22-7.05 (m, 1H), 7.05-6.78 (m, 2H), 6.78-6.55 (m, 2H), 6.40-6.38 (m, 1H), 6.11 (s, 2H), 4.78-4.35 (m, 4H), 4.13 (s, 2H), 4.05-3.72 (m, 6H), 3.55 (d, J=10.4 Hz, 6H), 3.36-3.15 (m, 3H), 3.15-2.79 (m, 5H), 2.81-2.56 (m, 2H), 2.51-2.05 (m, 4H), 2.03 (s, 1H), 1.96-1.70 (m, 6H), 1.52-1.50 (m, 5H), 1.50-1.27 (m, 11H), 1.27-1.01 (m, 5H), 0.89 (d, J=7.5 Hz, 2H).

### Example 112 Preparation of 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-4-{2-[(2R)-2-(2-methylphenyl)-4-[(1r,4r)-4-hydroxy-4-methylcyclohexyl]piperazin-1-yl]-7-azaspiro[3.5]nonan-7-yl}-N-[3-nitro-4-({[(1r,4r)-4-hydroxy-4-methylcyclohexyl]methyl}amino)benzenesulfonyl]benzamide (assumed)

Synthesis of 1-methyl-4-[(3R)-3-(2-methylphenyl)piperazin-1-yl]cyclohexan-1-ol (assumed): Into an 8 mL vial, were added (2R)-2-(2-methylphenyl)piperazine (180 mg, 1.0 mmol, 1.0 eq), 4-hydroxy-4-methylcyclohexan-1-one (130 mg, 1.0 mmol, 1.0 eq), DCE (6 mL), NaBH(OAc)₃ (649 mg, 3.0 mmol, 3.0 eq). The resulting mixture was stirred for overnight at 25° C. The reaction was quenched with water (10 mL) and then extracted with dichloromethane (3×10 mL). The combined organic layer was dried over anhydrous Na₂SO₄. After filtration, the filtrate was concentrated under vacuum. The residue was purified by Prep-TLC (dichloromethane/methanol=10:1) to give 1-methyl-4-[(3R)-3-(2-methylphenyl)piperazin-1-yl]cyclohexan-1-ol as a colorless oil (190 mg, 64.5%). LC-MS (ESI, m/z) M+1: 289.

Synthesis of (1R,4r)-1-methyl-4-((R)-3-(o-tolyl)piperazin-1-yl)cyclohexan-1-ol (assumed) &(1S,4s)-1-methyl-4-((R)-3-(o-tolyl)piperazin-1-yl)cyclohexan-1-ol (assumed): 1-methyl-4-[(3R)-3-(2-methylphenyl)piperazin-1-yl]cyclohexan-1-ol (800 mg) was purified by Chiral-SFC using the following conditions: CHIRAL ART Cellulose-SB, 3*25 cm, 5 um; Mobile Phase A: CO₂, Mobile Phase B: MeOH (0.1% 2 M NH₃-MeOH); Flow rate: 80 mL/min; Gradient: isocratic 30% B; Wave Length: 220 nm; RT(min): 2.52; RT(min): 3.74; Sample Solvent: MeOH—Preparative; Injection Volume: 2 mL; Number Of Runs: 15. Finally, (1R,4r)-1-methyl-4-((R)-3-(o-tolyl)piperazin-1-yl)cyclohexan-1-ol was obtained as a colorless oil (60 mg, 31.5%) and (1S,4s)-1-methyl-4-((R)-3-(o-tolyl)piperazin-1-yl)cyclohexan-1-ol was obtained as a colorless oil (20 mg, 10.5%).

Synthesis of 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-4-{2-[(2R)-2-(2-methylphenyl)-4-[(1r,4r)-4-hydroxy-4-methylcyclohexyl]piperazin-1-yl]-7-azaspiro[3.5]nonan-7-yl}-N-[3-nitro-4-({[(1r,4r)-4-hydroxy-4-methylcyclohexyl]methyl}amino)benzenesulfonyl]benzamide (assumed): Into an 8 mL vial, were added 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-N-[3-nitro-4-({[(1r,4r)-4-hydroxy-4-methylcyclohexyl]methyl}amino)benzenesulfonyl]-4-{2-oxo-7-azaspiro[3.5]nonan-7-yl}benzamide (70 mg, 0.08 mmol, 1.0 eq), MeOH (5 mL), (1r,4r)-1-methyl-4-[(3R)-3-(2-methylphenyl)piperazin-1-yl]cyclohexan-1-ol (25 mg, 0.08 mmol, 1.0 eq), ZnCl₂ (35.16 mg, 0.24 mmol, 3.0 eq), NaBH₃CN (16 mg, 0.24 mmol, 3.0 eq). The resulting mixture was stirred for 5 hours at 70° C. The reaction was quenched with water (10 mL) and then extracted with dichloromethane (3×10 mL). The combined organic layer was dried over anhydrous Na₂SO₄. After filtration, the filtrate was concentrated under vacuum. The residue was purified by Prep-TLC (dichloromethane/methanol=10:1) to give 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-4-{2-[(2R)-2-(2-methylphenyl)-4-[(1r,4r)-4-hydroxy-4-methylcyclohexyl]piperazin-1-yl]-7-azaspiro[3.5]nonan-7-yl}-N-[3-nitro-4-({[(1r,4r)-4-hydroxy-4-methylcyclohexyl]methyl}amino)benzenesulfonyl]benzamide as a yellow solid (8 mg, 8.5%). LC-MS (ESI, m/z) M+1: 1086. ¹H NMR (400 MHz, Methanol-d₄) δ 8.63-8.47 (m, 1H), 7.81 (d, J=12.5 Hz, 1H), 7.52-7.42 (m, 2H), 7.27-7.10 (m, 3H), 7.08-6.75 (m, 2H), 6.75-6.56 (m, 2H), 6.52 (s, 1H), 6.03 (dd, J=13.4, 3.5 Hz, 1H), δ 4.23 (s, 1H), 4.11-4.02 (m, 1H), 3.99-3.86 (m, 1H), 3.79-3.54 (m, 2H), 3.54-3.40 (m, 4H), 3.25 (d, J=6.6 Hz, 2H), 3.19-2.77 (m, 10H), 2.69-2.26 (m, 7H), 2.19-1.91 (m, 1H), 1.89-1.10 (m, 27H), 1.09-0.87 (m, 1H).

### Example 113 Preparation of 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-4-{2-[(2R)-2-(2-methylphenyl)-4-[(1s,4s)-4-hydroxy-4-methylcyclohexyl]piperazin-1-yl]-7-azaspiro[3.5]nonan-7-yl}-N-[3-nitro-4-({[(1r,4r)-4-hydroxy-4-methylcyclohexyl]methyl}amino)benzenesulfonyl]benzamide hydrochloride (assumed)

Synthesis of 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-4-{2-[(2R)-2-(2-methylphenyl)-4-[(1s,4s)-4-hydroxy-4-methylcyclohexyl]piperazin-1-yl]-7-azaspiro[3.5]nonan-7-yl}-N-[3-nitro-4-({[(1r,4r)-4-hydroxy-4-methylcyclohexyl]methyl}amino)benzenesulfonyl]benzamide hydrochloride (assumed): Into a 5 mL vial, were added 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-N-[3-nitro-4-({[(1r,4r)-4-hydroxy-4-methylcyclohexyl]methyl}amino)benzenesulfonyl]-4-{2-oxo-7-azaspiro[3.5]nonan-7-yl}benzamide ((70 mg, 0.08 mmol, 1.0 eq), (1s,4s)-1-methyl-4-[(3R)-3-(2-methylphenyl)piperazin-1-yl]cyclohexan-1-ol (25 mg, 0.08 mmol, 1.0 eq), MeOH (5 mL), ZnCl₂ (35.16 mg, 0.24 mmol, 3.0 eq), NaBH₃CN (16 mg, 0.24 mmol, 3.0 eq). The resulting mixture was stirred for 5 hours at 70° C. The reaction was quenched with water (10 mL) and then extracted with dichloromethane (3×10 mL). The combined organic layer was dried over anhydrous Na₂SO₄. After filtration, the filtrate was concentrated under vacuum. The residue was purified by Prep-TLC (dichloromethane/methanol=10:1) to give 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-4-{2-[(2R)-2-(2-methylphenyl)-4-[(1s,4s)-4-hydroxy-4-methylcyclohexyl]piperazin-1-yl]-7-azaspiro[3.5]nonan-7-yl}-N-[3-nitro-4-({[(1r,4r)-4-hydroxy-4-methylcyclohexyl]methyl}amino)benzenesulfonyl]benzamide hydrochloride as a yellow solid (15 mg, 15.5%). LC-MS (ESI, m/z) M+1-HCl: 1086. ¹H NMR (400 MHz, Methanol-d₄) δ 8.53 (dd, J=14.7, 2.2 Hz, 1H), 8.06 (t, J=9.3 Hz, 1H), 7.85-7.74 (m, 1H), 7.70 (dd, J=8.8, 3.1 Hz, 1H), 7.38 (dt, J=19.2, 6.6 Hz, 3H), 7.17 (d, J=12.1 Hz, 2H), 7.08-6.74 (m, 3H), 5.38 (d, J=10.7 Hz, 1H), 4.34-4.08 (m, 2H), 4.10-3.62 (m, 9H), 3.55-3.38 (m, 3H), 3.39-3.34 (m, 1H), 3.29-3.13 (m, 5H), 2.57 (d, J=5.3 Hz, 3H), 2.41-2.13 (m, 4H), 1.93-1.37 (m, 21H), 1.27 (dd, J=10.3, 2.2 Hz, 8H), 1.07 (s, 1H).

### Example 114 Preparation of 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-4-{2-[(2R)-2-(2-methylphenyl)-4-(oxan-4-yl)piperazin-1-yl]-7-azaspiro[3.5]nonan-7-yl}-N-[3-nitro-4-({[(1r,4r)-4-hydroxy-4-methylcyclohexyl]methyl}amino)benzenesulfonyl]benzamide (assumed)

Synthesis of (3R)-3-(2-methylphenyl)-1-(oxan-4-yl)piperazine: Into a 40 mL vial, were added (2R)-2-(2-methylphenyl)piperazine (85 mg, 0.5 mmol, 1.0 eq), tetrahydro-4H-pyran-4-one (48 mg, 0.5 mmol, 1.0 eq), DCE (4 mL), NaBH(OAc)₃ (306 mg, 1.5 mmol, 3.0 eq). The resulting mixture was stirred for overnight at 25° C. The reaction was quenched with water (10 mL) and then extracted with dichloromethane (3×10 mL). The combined organic layer was dried over anhydrous Na₂SO₄. After filtration, the filtrate was concentrated under vacuum. The residue was purified by Prep-TLC (dichloromethane/methanol=10:1) to give (3R)-3-(2-methylphenyl)-1-(oxan-4-yl)piperazine as a colorless oil (60 mg, 47.7%). LC-MS (ESI, m/z) M+1: 261.

Synthesis of 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-4-{2-[(2R)-2-(2-methylphenyl)-4-(oxan-4-yl)piperazin-1-yl]-7-azaspiro[3.5]nonan-7-yl}-N-[3-nitro-4-({[(1r,4r)-4-hydroxy-4-methylcyclohexyl]methyl}amino)benzenesulfonyl]benzamide (assumed): Into an 8 mL vial, were added 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-N-[3-nitro-4-({[(1r,4r)-4-hydroxy-4-methylcyclohexyl]methyl}amino)benzenesulfonyl]-4-{2-oxo-7-azaspiro[3.5]nonan-7-yl}benzamide (81 mg, 0.1 mmol, 1.0 eq), (3R)-3-(2-methylphenyl)-1-(oxan-4-yl)piperazine (26 mg, 0.1 mmol, 1.0 eq), MeOH (3 mL), ZnCl₂ (41 mg, 0.3 mmol, 3.0 eq), NaBH₃CN (19 mg, 0.3 mmol, 3.0 eq). The resulting mixture was stirred for 7 hours at 70° C. The reaction was quenched with water (5 mL) and then extracted with dichloromethane (3×10 mL). The combined organic layer was dried over anhydrous Na₂SO₄. After filtration, the filtrate was concentrated under vacuum. The residue was purified by Prep-TLC (dichloromethane/methanol=10:1) to give 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-4-{2-[(2R)-2-(2-methylphenyl)-4-(oxan-4-yl)piperazin-1-yl]-7-azaspiro[3.5]nonan-7-yl}-N-[3-nitro-4-({[(1r,4r)-4-hydroxy-4-methylcyclohexyl]methyl}amino)benzenesulfonyl]benzamide as a yellow solid (16 mg, 15.1%). LC-MS (ESI, m/z) M+1: 1059. ¹H NMR (400 MHz, Methanol-d₄) δ 8.49 (d, J=2.2 Hz, 1H), 7.88 (d, J=9.3 Hz, 1H), 7.49 (dd, J=9.3, 2.2 Hz, 2H), 7.22-7.12 (m, 3H), 7.08 (d, J=3.5 Hz, 1H), 6.96 (d, J=7.9 Hz, 1H), 6.70 (d, J=3.0 Hz, 2H), 6.49 (s, 1H), 6.02 (d, J=3.5 Hz, 1H), 4.29 (d, J=7.5 Hz, 2H), 4.05-3.86 (m, 3H), 3.81-3.6 (m, 2H), 3.58-3.45 (m, 5H), 3.44-3.37 (m, 2H), 3.26 (d, J=7.1 Hz, 2H), 3.21-2.96 (m, 2H), 2.84 (d, J=11.6 Hz, 1H), 2.61-2.45 (m, 3H), 2.39 (s, 3H), 1.94-1.78 (m, 7H), 1.76-1.65 (d, J=10.2 Hz, 4H), 1.62-1.38 (m, 5H), 1.37-1.20 (m, 10H), 1.20-1.08 (m, 1H), 0.99-0.83 (m, 2H).

### Example 115 Preparation of 4-{2-[(2S)-4-[(3,4-difluorophenyl)methyl]-2-(2-isopropoxyphenyl)piperazin-1-yl]-7-azaspiro[3.5]nonan-7-yl}-2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-N-[3-nitro-4-({[(1r,4r)-4-hydroxy-4-methylcyclohexyl]methyl}amino)benzenesulfonyl]benzamide (assumed)

Synthesis of (3S)-1-[(3,4-difluorophenyl)methyl]-3-(2-isopropoxyphenyl)piperazine (assumed): Into an 8-mL sealed-tube, were placed (2S)-2-(2-isopropoxyphenyl)piperazine (assumed) (100 mg, 0.5 mmol, 1.0 eq), 3,4-difluorobenzaldehyde (65 mg, 0.5 mmol, 1.0 eq), MeOH (2 mL), ZnCl₂ (186 mg, 1.4 mmol, 3.0 eq), NaBH₃CN (86 mg, 1.4 mmol, 3.0 eq). The resulting solution was stirred for 4 hours at 25° C. The reaction was then quenched by the addition of water (20 mL) and then extracted with dichloromethane/methanol=10:1 (2×20 mL). The combined organic layer was washed with brine (2×40 mL) and dried over anhydrous Na₂SO₄. After filtration, the filtrate was concentrated under vacuum. The crude residue was purified onto a silica gel column and eluted with dichloromethane/methanol=10:1 to give (3S)-1-[(3,4-difluorophenyl)methyl]-3-(2-isopropoxyphenyl)piperazine (assumed) as an off-white solid (60 mg, 38.2%). LC-MS (ESI, m/z) M+1: 347.

Synthesis of 4-{2-[(2S)-4-[(3,4-difluorophenyl)methyl]-2-(2-isopropoxyphenyl)piperazin-1-yl]-7-azaspiro[3.5]nonan-7-yl}-2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-N-[3-nitro-4-({[(1r,4r)-4-hydroxy-4-methylcyclohexyl]methyl}amino)benzenesulfonyl]benzamide (assumed): Into an 8-mL sealed-tube, were placed (3S)-1-[(3,4-difluorophenyl)methyl]-3-(2-isopropoxyphenyl)piperazine (assumed) (35 mg, 0.1 mmol, 1.0 eq), 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-N-[3-nitro-4-({[(1r,4r)-4-hydroxy-4-methylcyclohexyl]methyl}amino)benzenesulfonyl]-4-{2-oxo-7-azaspiro[3.5]nonan-7-yl}benzamide (82 mg, 0.1 mmol, 1.0 eq), MeOH (1 mL), ZnCl₂ (41 mg, 0.3 mmol, 3.0 eq), NaBH₃CN (19 mg, 0.3 mmol, 3.0 eq). The resulting solution was stirred for 3 hours at 70° C. The reaction was then quenched by the addition of water (20 mL) and then extracted with dichloromethane/methanol=10:1 (2×30 mL). The combined organic layer was washed with brine (2×20 mL) and dried over anhydrous Na₂SO₄. After filtration, the filtrate was concentrated under vacuum. The residue was purified by Prep-TLC (dichloromethane/methanol=15:1) to give 4-{2-[(2S)-4-[(3,4-difluorophenyl)methyl]-2-(2-isopropoxyphenyl)piperazin-1-yl]-7-azaspiro[3.5]nonan-7-yl}-2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-N-[3-nitro-4-({[(1r,4r)-4-hydroxy- 4-methylcyclohexyl]methyl}amino)benzenesulfonyl]benzamide (assumed) as a light yellow solid (25 mg, 21.6%). LC-MS (ESI, m/z) M+1: 1145. ¹H NMR (400 MHz, Methanol-d₄) δ 8.53-8.46 (m, 1H), 7.87 (d, J=9.0 Hz, 1H), 7.55-7.40 (m, 2H), 7.32-7.11 (m, 4H), 7.07 (d, J=3.6 Hz, 1H), 7.03-6.86 (m, 3H), 6.72-6.63 (m, 2H), 6.49 (s, 1H), 6.02 (d, J=3.4 Hz, 1H), 4.61 (p, J=6.7, 6.0 Hz, 1H), 4.31-4.26 (m, 2H), 4.16-4.12 (m, 1H), 3.93-3.89 (m, 1H), 3.73-3.67 (m, 1H), 3.59-3.55 (m, 2H), 3.52-3.45 (m, 2H), 3.27-3.09 (m, 6H), 2.98-2.92 (m, 1H), 2.68-2.63 (m, 1H), 2.48-2.28 (m, 3H), 2.04-1.93 (m, 1H), 1.87-1.81 (m, 2H), 1.76-1.65 (m, 5H), 1.57-1.49 (m, 5H), 1.44-1.40 (m, 2H), 1.39-1.37 (m, 2H), 1.34-1.31 (m, 4H), 1.29-1.22 (m, 9H).

### Example 116 Preparation of 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-4-{2-[(2S)-2-(2-isopropoxyphenyl)-4-({4-[(3R)-3-methylmorpholin-4-yl]phenyl}methyl)piperazin-1-yl]-7-azaspiro[3.5]nonan-7-yl}-N-[3-nitro-4-({[(1r,4r)-4-hydroxy-4-methylcyclohexyl]methyl}amino)benzenesulfonyl]benzamide (assumed)

Synthesis of 4-[(3R)-3-methylmorpholin-4-yl]benzaldehyde: Into a 100 mL round-bottom flask, were placed 4-fluoro-benzaldehyde (5.0 g, 40.3 mmol, 1.0 eq), (3R)-3-methylmorpholine (8.2 g, 80.6 mmol, 2.0 eq), K₂CO₃ (11.1 g, 80.6 mmol, 2.0 eq), DMSO (50 mL). The resulting solution was stirred for 4 hours at 130° C. The reaction was then quenched by the addition of water (50 mL) and extracted with ethyl acetate (2×50 mL). The combined organic layer was washed with water (2×50 mL), brine (2×50 mL) and over anhydrous Na₂SO₄. After filtration, the filtrate was concentrated under vacuum. The crude residue was applied onto a silica gel column and eluted with ethyl acetate/petroleum ether=1:3 to give 4-[(3R)-3-methylmorpholin-4-yl]benzaldehyde as a yellow solid (1.5 g, 18.1%). LC-MS (ESI, m/z) M+1: 206. ¹H NMR (300 MHz, DMSO-d₆) δ 9.72 (s, 1H), 7.86-7.62 (m, 2H), 7.13-6.85 (m, 2H), 4.17-4.06 (m, 1H), 3.99-3.91 (m, 1H), 3.74 (dt, J=11.4, 1.2 Hz, 1H), 3.67 (dd, J=11.4, 3.0 Hz, 1H), 3.56 (d, J=2.7 Hz, 1H), 3.54-3.51 (m, 1H), 3.10 (td, J=13.2, 4.1 Hz, 1H), 1.11 (d, J=6.6 Hz, 3H).

Synthesis of (3R)-4-(4-{[(3S)-3-(2-isopropoxyphenyl)piperazin-1-yl]methyl}phenyl)-3-methylmorpholine (assumed): Into an 8-mL sealed tube purged and maintained with an inert atmosphere of nitrogen, were placed 4-[(3R)-3-methylmorpholin-4-yl]benzaldehyde (75 mg, 0.4 mmol, 1.0 eq), (2S)-2-(2-isopropoxyphenyl)piperazine (assumed) (81 mg, 0.4 mmol, 1.0 eq), NaBH(OAc)₃ (232 mg, 1.1 mmol, 3.0 eq), DCE (1 mL). The resulting solution was stirred for 2 hours at 25° C. The reaction was then quenched by the addition of water (10 mL) and extracted with ethyl acetate (2×10 mL). The combined organic layer was washed with brine (2×10 mL) and dried over anhydrous Na₂SO₄. After filtration, the filtrate was concentrated under vacuum. The crude residue was applied onto a silica gel column and eluted with dichloromethane/methanol=10:1 to give (3R)-4-(4-{[(3S)-3-(2-isopropoxyphenyl)piperazin-1-yl]methyl}phenyl)-3-methylmorpholine (assumed) as a white solid (90 mg, 60.1%). LC-MS (ESI, m/z) M+1: 410. ¹H NMR (300 MHz, Chloroform-d) δ 7.52 (d, J=7.5 Hz, 1H), 7.28-7.19 (m, 4H), 6.86 (dd, J=8.7, 2.1 Hz, 3H), 4.59 (dd, J=12.3, 6.0 Hz, 1H), 4.55-4.49 (m, 1H), 3.99 (dt, J=11.1, 3.3 Hz, 1H), 3.90-3.82 (m, 1H), 3.72 (dt, J=11.1, 4.2 Hz, 4H), 3.57 (d, J=12.9 Hz, 1H), 3.30-3.25 (m, 2H), 3.14 (dd, J=8.4, 3.9 Hz, 2H), 3.12-2.97 (m, 1H), 2.60 (t, J=13.2 Hz, 1H), 2.47 (t, J=11.1 Hz, 1H), 1.38 (d, J=6.0 Hz, 3H), 1.26 (d, J=6.0 Hz, 3H), 1.09 (d, J=6.3 Hz, 3H).

Synthesis of 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-4-{2-[(2S)-2-(2-isopropoxyphenyl)-4-({4-[(3R)-3-methylmorpholin-4-yl]phenyl}methyl)piperazin-1-yl]-7-azaspiro[3.5]nonan-7-yl}-N-[3-nitro-4-({[(1r,4r)-4-hydroxy-4-methylcyclohexyl]methyl}amino)benzenesulfonyl]benzamide (assumed): Into an 8-mL sealed tube, were placed (3R)-4-(4-{[(3S)-3-(2-isopropoxyphenyl)piperazin-1-yl]methyl}phenyl)-3-methylmorpholine (assumed) (80 mg, 0.2 mmol, 1.0 eq), 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-N-[3-nitro-4-({[(1r,4r)-4-hydroxy-4-methylcyclohexyl]methyl}amino)benzenesulfonyl]-4-{2-oxo-7-azaspiro[3.5]nonan-7-yl}benzamide (159 mg, 0.2 mmol, 1.0 eq), ZnCl₂ (80 mg, 0.6 mmol, 3.0 eq), NaBH₃CN (37 mg, 0.6 mmol, 3.0 eq), MeOH (1 mL). The resulting solution was stirred for 6 hours at 70° C. The resulting mixture was filtered, the filtrate was concentrated under vacuum. The crude residue was applied onto a silica gel column and eluted with dichloromethane/methanol=15:1 to give 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-4-{2-[(2S)-2-(2-isopropoxyphenyl)-4-({4-[(3R)-3-methylmorpholin-4-yl]phenyl}methyl)piperazin-1-yl]-7-azaspiro[3.5]nonan-7-yl}-N-[3-nitro-4-({[(1r,4r)-4-hydroxy-4-methylcyclohexyl]methyl}amino)benzenesulfonyl]benzamide (assumed) as a yellow solid (3.4 mg, 1.4%). LC-MS (ESI, m/z) M+1: 1208. ¹H NMR (300 MHz, Chloroform-d) δ 12.59 (s, 1H), 8.65 (d, J=2.1 Hz, 1H), 8.45 (dd, J=12.3, 6.8 Hz, 2H), 8.11 (d, J=9.3 Hz, 1H), 7.85-7.76 (m, 1H), 7.43 (s, 1H), 7.20 (d, J=8.4 Hz, 2H), 7.10 (t, J=3.0 Hz, 1H), 6.83 (d, J=8.7 Hz, 5H), 6.71 (d, J=9.3 Hz, 1H), 6.64 (s, 1H), 6.43 (s, 1H), 6.11 (s, 1H), 4.52 (d, J=9.9 Hz, 3H), 4.45 (dd, J=11.1, 5.0 Hz, 1H), 3.98 (d, J=12.0 Hz, 4H), 3.86 (d, J=11.7 Hz, 2H), 3.72 (s, 4H), 3.48 (td, J=21.9, 21.3, 11.2 Hz, 6H), 3.21 (t, J=6.0 Hz, 2H), 3.08 (d, J=17.1 Hz, 9H), 2.98-2.91 (m, 3H), 2.68 (d, J=12.3 Hz, 1H), 2.29 (br, 1H), 2.16 (s, 1H), 1.87 (d, J=12.0 Hz, 2H), 1.77 (d, J=13.2 Hz, 4H), 1.56-1.43 (m, 2H), 1.43-1.30 (m, 6H), 1.28 (s, 5H), 1.19 (d, J=10.5 Hz, 1H), 1.07 (s, 4H).

### Example 117 Preparation of 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-4-{2-[(2S)-4-{2H,3H-[1,4]dioxino[2,3-b]pyridin-7-ylmethyl}-2-(2-isopropoxyphenyl)piperazin-1-yl]-7-azaspiro[3.5]nonan-7-yl}-N-[3-nitro-4-({[(1r,4r)-4-hydroxy-4-methylcyclohexyl]methyl}amino)benzenesulfonyl]benzamide (assumed)

Synthesis of (3S)-1-{2H,3H-[1,4]dioxino[2,3-b]pyridin-7-ylmethyl}-3-(2-isopropoxyphenyl)piperazine (assumed): Into an 8 mL vial were added 2H,3H-[1,4]dioxino[2,3-b]pyridine-7-carbaldehyde (80 mg, 0.5 mmol, 1.0 eq), (2S)-2-(2-isopropoxyphenyl)piperazine (107 mg, 0.5 mmol, 1.0 eq), NaBH₃CN (152 mg, 2.4 mmol, 5.0 eq), ZnCl₂ (330 mg, 2.4 mmol, 5.0 eq) and MeOH (1 mL). The resulting mixture was stirred for 2 hours at 25° C. The crude residue was purified by Prep-TLC (CH₂Cl₂/MeOH=10:1) to give (3S)-1-{2H,3H-[1,4]dioxino[2,3-b]pyridin-7-ylmethyl}-3-(2-isopropoxyphenyl)piperazine (assumed) as a white solid (63 mg, 35.2%). ¹H NMR (300 MHz, Chloroform-d) δ 7.77 (d, J=16.4 Hz, 1H), 7.37 (d, J=7.6 Hz, 1H), 7.08-6.82 (m, 2H), 4.78-4.66 (m, 1H), 4.65 (s, 2H), 4.49-4.44 (m, 2H), 4.29 (d, J=4.3 Hz, 2H), 3.63-3.67 (m, 3H), 3.30 (s, 1H), 3.02-3.05 (m, 2H), 2.90-2.98 (m, 2H), 2.69 (s, 1H), 1.54-1.30 (m, 6H).

Synthesis of 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-4-{2-[(2S)-4-{2H,3H-[1,4]dioxino[2,3-b]pyridin-7-ylmethyl}-2-(2-isopropoxyphenyl)piperazin-1-yl]-7-azaspiro[3.5]nonan-7-yl}-N-[3-nitro-4-({[(1r,4r)-4-hydroxy-4-methylcyclohexyl]methyl}amino)benzenesulfonyl]benzamide (assumed): Into an 8 mL vial were added (3S)-1-{2H,3H-[1,4]dioxino[2,3-b]pyridin-7-ylmethyl}-3-(2-isopropoxyphenyl)piperazine (assumed) (40 mg, 0.1 mmol, 1.0 eq), 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-N-[3-nitro-4-({[(1r,4r)-4-hydroxy-4-methylcyclohexyl]methyl}amino)benzenesulfonyl]-4-{2-oxo-7-azaspiro[3.5]nonan-7-yl}benzamide (88 mg, 0.1 mmol, 1.0 eq), NaBH₃CN (34 mg, 0.5 mmol, 5.0 eq), ZnCl₂ (74 mg, 0.5 mmol, 5.0 eq) and MeOH (2 mL) at 70° C. The resulting mixture was stirred for 4 hours. The reaction was quenched by the addition of water (2 mL) and extracted with CH₂Cl₂ (3×5 mL). The combined organic layer was washed with brine (5 mL), dried over anhydrous Na₂SO₄. After filtration, the filtrate was concentrated under vacuum. The residue was purified by Prep-TLC (CH₂Cl₂/MeOH=17:1) to give 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-4-{2-[(2S)-4-{2H,3H-[1,4]dioxino[2,3-b]pyridin-7-ylmethyl}-2-(2-isopropoxyphenyl)piperazin-1-yl]-7-azaspiro[3.5]nonan-7-yl}-N-[3-nitro-4-({[(1r,4r)-4-hydroxy-4-methylcyclohexyl]methyl}amino)benzenesulfonyl]benzamide (assumed) as a yellow solid (8 mg, 6.3%). LC-MS (ESI, m/z) M+1: 1168. ¹H NMR (300 MHz, Chloroform-d) δ 12.59 (s, 1H), 8.65 (d, J=2.2 Hz, 1H), 8.47 (s, 2H), 8.11 (d, J=10.6 Hz, 2H), 7.80 (d, J=9.1 Hz, 1H), 7.74 (s, 1H), 7.16 (s, 1H), 7.10 (t, J=2.9 Hz, 1H), 6.83 (d, J=9.2 Hz, 2H), 6.70 (d, J=9.3 Hz, 1H), 6.63 (s, 1H), 6.43 (s, 1H), 6.11 (s, 1H), 4.71 (s, 1H), 4.52 (d, J=11.4 Hz, 3H), 4.48-4.36 (m, 3H), 4.25 (s, 2H), 4.07-3.81 (m, 2H), 3.74-3.37 (m, 7H), 3.21 (t, J=6.1 Hz, 3H), 3.07-3.01 (m, 6H), 1.86 (d, J=13.3 Hz, 3H), 1.77 (d, J=13.4 Hz, 6H), 1.60-1.38 (m, 12H), 1.28 (d, J=2.0 Hz, 9H).

### Example 118 Preparation of 4-{2-[(2R)-4-[(5,6-dimethoxypyridin-3-yl)methyl]-2-(2-methylphenyl)piperazin-1-yl]-7-azaspiro[3.5]nonan-7-yl}-2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-N-[3-nitro-4-({[(1r,4r)-4-hydroxy-4-methylcyclohexyl]methyl}amino)benzenesulfonyl]benzamide (assumed)

Synthesis of 5-bromo-2,3-dimethoxypyridine: Into a 50-mL round-bottom flask, were placed 2,3-dimethoxypyridine (8.0 g, 57.5 mmol, 1.0 eq), Br₂ (10.1 g, 63.2 mmol, 1.1 eq), dichloromethane (80 mL). The resulting mixture was stirred for 2 hours at 0° C. The reaction was then quenched by the addition of water (50 mL) and extracted with ethyl acetate (50 mL). The combined organic layer was washed with water (50 mL×2), brine (50 mL×2) and dried over Na₂SO₄. After filtration, the resulting mixture was concentrated under vacuum. The crude residue was applied onto a silica gel column and eluted with ethyl acetate/petroleum ether=1:2 to give 5-bromo-2,3-dimethoxypyridine as an off-white oil (5.0 g, 40.0%). LC-MS (ESI, m/z) M+1: 218. ¹H NMR (300 MHz, Chloroform-d) δ 7.73 (d, J=2.1 Hz, 1H), 7.09 (d, J=2.1 Hz, 1H), 3.95 (s, 3H), 3.83 (s, 3H).

Synthesis of 5,6-dimethoxypyridine-3-carbaldehyde: Into a 250 mL 3-necked round-bottom flask, were placed 5-bromo-2,3-dimethoxypyridine (4.7 g, 21.6 mmol, 1.0 eq), THE (50 mL). After that, n-BuLi (1.1 eq) was added at −78° C. The resulting mixture was stirred for 30 minutes at −78° C. To the above mixture was added DMF (2.2 g, 30.2 mmol, 1.4 eq) at −78° C. The resulting mixture was stirred for additional 30 minutes at −78° C. The reaction was then quenched by the addition of water (200 mL) and extracted with ethyl acetate (2×100 mL). The combined organic layer was washed with brine (2×100 mL), dried over anhydrous Na₂SO₄. After filtration, the filtrate was concentrated under vacuum. The crude residue was purified onto a silica gel column and eluted with ethyl acetate/petroleum ether=1:1 to give 5,6-dimethoxypyridine-3-carbaldehyde as a light pink solid (2.7 g, 74.9%). LC-MS (ESI, m/z) M+1: 168. ¹H NMR (300 MHz, DMSO-d₆) δ 9.95 (s, 1H), 8.35 (d, J=1.8 Hz, 1H), 7.53 (d, J=1.8 Hz, 1H), 3.99 (s, 3H), 3.87 (s, 3H).

Synthesis of (3R)-1-[(5,6-dimethoxypyridin-3-yl)methyl]-3-(2-methylphenyl)piperazine (assumed): Into a 40 mL vial were added 5,6-dimethoxypyridine-3-carbaldehyde (100 mg, 0.6 mmol, 1.0 eq), (2R)-2-(2-methylphenyl)piperazine (assumed) (105 mg, 0.6 mmol, 1.0 eq), NaBH₃CN (1878 mg, 3.0 mmol, 5.0 eq), ZnCl₂ (196 mg, 3.0 mmol, 5.0 eq) and MeOH (2 mL). The resulting mixture was stirred for 4 hours at 25° C. The reaction was quenched by the addition of water (2 mL) and extracted with EtOAc (3×5 mL). The combined organic layer was washed with brine (5 mL), dried over anhydrous Na₂SO₄. After filtration, the filtrate was concentrated under vacuum. The crude residue was purified by Prep-TLC (CH₂Cl₂/MeOH=10:1) to give (3R)-1-[(5,6-dimethoxypyridin-3-yl)methyl]-3-(2-methylphenyl)piperazine (assumed) as an off-white solid (75 mg, 38.3%). ¹H NMR (300 MHz, Chloroform-d) δ 7.57 (s, 1H), 7.53-7.38 (m, 1H), 7.27-7.18 (m, 2H), 7.15 (d, J=5.2 Hz, 2H), 4.43-4.24 (m, 1H), 3.97 (s, 3H), 3.89 (s, 3H), 3.75-3.66 (m, 1H), 3.65-3.49 (m, 2H), 3.37 (s, 2H), 2.96 (dd, J=19.1, 12.3 Hz, 2H), 2.62 (s, 1H), 2.45 (t, J=11.6 Hz, 1H), 2.32 (s, 3H).

Synthesis of 4-{2-[(2R)-4-[(5,6-dimethoxypyridin-3-yl)methyl]-2-(2-methylphenyl)piperazin-1-yl]-7-azaspiro[3.5]nonan-7-yl}-2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-N-[3-nitro-4-({[(1r,4r)-4-hydroxy-4-methylcyclohexyl]methyl}amino)benzenesulfonyl]benzamide (assumed): Into an 8 mL vial were added (3R)-1-[(5,6-dimethoxypyridin-3-yl)methyl]-3-(2-methylphenyl)piperazine (assumed) (40 mg, 0.1 mmol, 1.0 eq), 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-N-[3-nitro-4-({[(1r,4r)-4-hydroxy-4-methylcyclohexyl]methyl}amino)benzenesulfonyl]-4-{2-oxo-7-azaspiro[3.5]nonan-7-yl}benzamide (99 mg, 0.1 mmol, 1.0 eq), NaBH₃CN (39 mg, 0.6 mmol, 5.0 eq), ZnCl₂ (83 mg, 0.6 mmol, 5.0 eq) and MeOH (2 mL). The resulting mixture was stirred for 4 hours at 70° C. The reaction was quenched by the addition of water (2 mL) and extracted with EtOAc (3×5 mL). The combined organic layer was washed with brine (5 mL), dried over anhydrous Na₂SO₄. After filtration, the filtrate was concentrated under vacuum. The crude residue was purified by Prep-TLC (CH₂Cl₂/MeOH=15:1) to give 4-{2-[(2R)-4-[(5,6-dimethoxypyridin-3-yl)methyl]-2-(2-methylphenyl)piperazin-1-yl]-7-azaspiro[3.5]nonan-7-yl}-2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-N-[3-nitro-4-({[(1r,4r)-4-hydroxy- 4-methylcyclohexyl]methyl}amino)benzenesulfonyl]benzamide (assumed)(16 mg, 11.6%) as a yellow solid. LC-MS (ESI, m/z) M+1: 1126. ¹H NMR (300 MHz, Chloroform-d) δ 12.59 (s, 1H), 8.65 (d, J=2.2 Hz, 1H), 8.46 (d, J=5.4 Hz, 2H), 8.11 (d, J=9.1 Hz, 1H), 7.80 (d, J=9.6 Hz, 1H), 7.59 (s, 1H), 7.43 (s, 1H), 7.10 (t, J=3.0 Hz, 3H), 6.84 (d, J=9.3 Hz, 1H), 6.76-6.65 (m, 1H), 6.63 (s, 1H), 6.44 (d, J=2.4 Hz, 1H), 6.11 (s, 1H), 4.63-4.40 (m, 2H), 4.01 (s, 3H), 3.89 (s, 3H), 3.59-3.35 (m, 6H), 3.21 (t, J=6.1 Hz, 4H), 3.15-2.87 (m, 9H), 2.47-2.24 (m, 6H), 1.86 (d, J=13.7 Hz, 5H), 1.77 (d, J=13.1 Hz, 6H), 1.28 (s, 7H), 1.25-0.99 (m, 3H).

### Example 119 Preparation of N-((4-((((1r,4r)-4-hydroxy-4-methylcyclohexyl)methyl)amino)-3-nitrophenyl)sulfonyl)-4-(2-((S)-2-(2-isopropoxyphenyl)-4-((5-(methoxy-d3)-6-morpholinopyridin-3-yl)methyl)piperazin-1-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide (assumed)

Synthesis of (S)-4-(5-((3-(2-isopropoxyphenyl)piperazin-1-yl)methyl)-3-(methoxy-d3)pyridin-2-yl)morpholine (assumed): Into a 40-mL sealed-tube, were placed (S)-2-(2-isopropoxyphenyl)piperazine (assumed) (150 mg, 0.5 mmol, 1.0 eq), 5-(methoxy-d3)-6-morpholinonicotinaldehyde (118 mg, 0.5 mmol, 1.1 eq), DCE (4 mL), NaBH(OAc)₃ (307 mg, 1.4 mmol, 3.0 eq). The resulting solution was stirred for 2 hours at 25° C. The reaction was then quenched by the addition of water (40 mL) and extracted with ethyl acetate (2×40 mL). The combined organic layer was washed with brine (2×40 mL) and dried over anhydrous Na₂SO₄. After filtration, the filtrate was concentrated under vacuum. The crude residue was purified onto a silica gel column and eluted with ethyl acetate/petroleum ether=1:2 to give (S)-4-(5-((3-(2-isopropoxyphenyl)piperazin-1-yl)methyl)-3-(methoxy-d3)pyridin-2-yl)morpholine (assumed) as a light yellow solid (110 mg, 44.1%). LC-MS (ESI, m/z) M+1: 430.

Synthesis of N-((4-((((1r,4r)-4-hydroxy-4-methylcyclohexyl)methyl)amino)-3-nitrophenyl)sulfonyl)-4-(2-((S)-2-(2-isopropoxyphenyl)-4-((5-(methoxy-d3)-6-morpholinopyridin-3-yl)methyl)piperazin-1-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide (assumed): Into an 8 mL vial, were added N-((4-((((1r,4r)-4-hydroxy-4-methylcyclohexyl)methyl)amino)-3-nitrophenyl)sulfonyl)-4-(2-oxo-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide (87 mg, 0.1 mmol, 0.7 eq), (S)-4-(5-((3-(2-isopropoxyphenyl)piperazin-1-yl)methyl)-3-(methoxy-d3)pyridin-2-yl)morpholine (assumed) (45 mg, 0.1 mmol, 1.0 eq), NaBH₃CN (41 mg, 0.7 mmol, 5.0 eq), ZnCl₂ (90 mg, 0.7 mmol, 5.0 eq) and MeOH (2 mL). The resulting mixture was stirred for 5 hours at 70° C. The reaction was quenched by the addition of water (2 mL) and then extracted with CH₂Cl₂ (3×3 mL). The combined organic layer was washed with brine (3 mL), dried over anhydrous Na₂SO₄. After filtration, the filtrate was concentrated under vacuum. The residue was purified by Prep-TLC (CH₂Cl₂/MeOH=20:1) to give N-((4-((((1r,4r)-4-hydroxy-4-methylcyclohexyl)methyl)amino)-3-nitrophenyl)sulfonyl)-4-(2-((S)-2-(2-isopropoxyphenyl)-4-((5-(methoxy-d3)-6-morpholinopyridin-3-yl)methyl)piperazin-1-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide (assumed) as a yellow solid (15 mg, 15.9%). LC-MS (ESI, m/z) M+1: 1128. ¹H NMR (300 MHz, Chloroform-d) δ 12.57 (s, 1H), 8.65 (s, 2H), 8.46 (s, 2H), 8.40 (s, 1H), 8.11 (d, J=9.6 Hz, 2H), 7.80 (s, 2H), 7.07 (d, J=16.5 Hz, 3H), 6.98 (s, 1H), 6.85 (d, J=8.1 Hz, 4H), 6.71 (d, J=9.3 Hz, 2H), 6.62 (s, 1H), 6.43 (s, 2H), 6.11 (s, 2H), 4.61-4.50 (m, 1H), 4.00 (s, 1H), 3.86 (s, 8H), 3.58 (s, 2H), 3.51 (d, J=9.0 Hz, 4H), 3.38 (s, 8H), 3.21 (t, J=6.3 Hz, 3H), 3.06 (s, 11H), 1.75 (s, 2H), 1.51 (d, J=12.3 Hz, 1H), 1.37 (dd, J=14.7, 9.0 Hz, 9H), 0.89 (d, J=8.1 Hz, 3H).

### Example 120 Preparation of 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-4-[(1S,2R)-2-[(2S)-2-(2-isopropoxyphenyl)-4-{[5-methoxy-6-(morpholin-4-yl)pyridin-3-yl]methyl}piperazin-1-yl]-1-methyl-7-azaspiro[3.5]nonan-7-yl]-N-[3-nitro-4-({[(1r,4r)-4-hydroxy-4-methylcyclohexyl]methyl}amino)benzenesulfonyl]benzamide (assumed)

Synthesis of 2-bromo-4-{1-methyl-2-oxo-7-azaspiro[3.5]nonan-7-yl}benzoic acid: Into a 100 mL round-bottom flask, were placed methyl 2-bromo-4-{1-methyl-2-oxo-7-azaspiro[3.5]nonan-7-yl}benzoate (2.5 g, 6.8 mmol, 1.0 eq), methanol (12.5 mL), 1,4-dioxane (12.5 mL), NaOH (1.1 g, 27.3 mmol, 4.0 eq). The resulting mixture was stirred for 16 hours at 45° C. The resulting mixture was then diluted with water (30 mL) and acidified to pH=5 with HCl (1 M). The precipitated solids were collected by filtration and dried under infrared light to give 2-bromo-4-{1-methyl-2-oxo-7-azaspiro[3.5]nonan-7-yl}benzoic acid as a yellow solid (2.0 g, crude). LC-MS (ESI, m/z) M+1: 352.

Synthesis of 2-bromo-4-{1-methyl-2-oxo-7-azaspiro[3.5]nonan-7-yl}-N-[3-nitro-4-({[(1r,4r)-4-hydroxy-4-methylcyclohexyl]methyl}amino)benzenesulfonyl]benzamide: Into a 40 mL vial, were placed 2-bromo-4-{1-methyl-2-oxo-7-azaspiro[3.5]nonan-7-yl}benzoic acid (1.0 g, 2.9 mmol, 1.0 eq), 3-nitro-4-({[(1r,4r)-4-hydroxy-4-methylcyclohexyl]methyl}amino)benzenesulfonamide (970 mg, 2.8 mmol, 1.0 eq), dichloromethane (10 mL), EDCl (1.1 g, 5.7 mmol, 2.0 eq), DMAP (1.4 g, 11.4 mmol, 4.0 eq). The resulting mixture was stirred overnight at 25° C. The resulting mixture was concentrated under vacuum. The crude residue was applied onto a silica gel column and eluted with dichloromethane/ethyl acetate (included 20% of methanol)=1:1 to give 2-bromo-4-{1-methyl-2-oxo-7-azaspiro[3.5]nonan-7-yl}-N-[3-nitro-4-({[(1r,4r)-4-hydroxy-4-methylcyclohexyl]methyl}amino)benzenesulfonyl]benzamide as a yellow solid (1.2 g, 62.4%). LC-MS (ESI, m/z) M+1: 677.

Synthesis of 4-{1-methyl-2-oxo-7-azaspiro[3.5]nonan-7-yl}-N-[3-nitro-4-({[(1r,4r)-4-hydroxy-4-methylcyclohexyl]methyl}amino)benzenesulfonyl]-2-[(3R,8S)-15-{[2-(trimethylsilyl)ethoxy]methyl}-2,5-dioxa-9,15,17-triazatetracyclo[8.7.0.0\hat{ }{3,8}.0\hat{ }{12,16}]heptadeca-1(10),11,13,16-tetraen-9-yl]benzamide: Into an 8-mL sealed tube purged and maintained with an inert atmosphere of nitrogen, were placed 2-bromo-4-{1-methyl-2-oxo-7-azaspiro[3.5]nonan-7-yl}-N-[3-nitro-4-({[(1r,4r)-4-hydroxy-4-methylcyclohexyl]methyl}amino)benzenesulfonyl]benzamide (800 mg, 1.2 mmol, 1.0 eq), (3R,8S)-15-{[2-(trimethylsilyl)ethoxy]methyl}-2,5-dioxa-9,15,17-triazatetracyclo[8.7.0.0\hat{ }{3,8}.0\hat{ }{12,16}]heptadeca-1(10),11,13,16-tetraene (427 mg, 1.2 mmol, 1.0 eq), N₁,N₂-di-o-tolyloxalamide (170 mg, 0.7 mmol, 0.6 eq), K₂CO₃ (490 mg, 3.5 mmol, 3.0 eq), Cul (135 mg, 0.7 mmol, 0.6 eq), DMSO (1 mL). The resulting solution was stirred for 2 hours at 100° C. The reaction was then quenched by the addition of water (30 mL) and extracted with ethyl acetate (2×30 mL). The combined organic layer was washed with brine (2×30 mL) and dried over anhydrous Na₂SO₄. After filtration, the filtrate was concentrated under vacuum. The crude residue was applied onto a silica gel column and eluted with dichloromethane/methanol=10:1 to give 4-{1-methyl-2-oxo-7-azaspiro[3.5]nonan-7-yl}-N-[3-nitro-4-({[(1r,4r)-4-hydroxy-4-methylcyclohexyl]methyl}amino)benzenesulfonyl]-2-[(3R,8S)-15-{[2-(trimethylsilyl)ethoxy]methyl}-2,5-dioxa-9,15,17-triazatetracyclo[8.7.0.0\hat{ }{3,8}.0\hat{ }{12,16}]heptadeca-1(10),11,13,16-tetraen-9-yl]benzamide as a yellow solid (300 mg, 26.5%). LC-MS (ESI, m/z) M+1: 958.

Synthesis of 4-[(1R,2R)-2-[(2S)-2-(2-isopropoxyphenyl)-4-{[5-methoxy-6-(morpholin-4-yl)pyridin-3-yl]methyl}piperazin-1-yl]-1-methyl-7-azaspiro[3.5]nonan-7-yl]-N-[3-nitro-4-({[(1r,4r)-4-hydroxy-4-methylcyclohexyl]methyl}amino)benzenesulfonyl]-2-[(3R,8S)-15-{[2-(trimethylsilyl)ethoxy]methyl}-2,5-dioxa-9,15,17-triazatetracyclo[8.7.0.0\hat{ }{3,8}.0\hat{ }{12,16}]heptadeca-1(10),11,13,16-tetraen-9-yl]benzamide (assumed) & 4-[(1S,2R)-2-[(2S)-2-(2-isopropoxyphenyl)-4-{[5-methoxy-6-(morpholin-4-yl)pyridin-3-yl]methyl}piperazin-1-yl]-1-methyl-7-azaspiro[3.5]nonan-7-yl]-N-[3-nitro-4-({[(1r,4r)-4-hydroxy-4-methylcyclohexyl]methyl}amino)benzenesulfonyl]-2-[(3R,8S)-15-{[2-(trimethylsilyl)ethoxy]methyl}-2,5-dioxa-9,15,17-triazatetracyclo[8.7.0.0\hat{ }{3,8}.0\hat{ }{12,16}]heptadeca-1(10),11,13,16-tetraen-9-yl]benzamide (assumed): Into an 8-mL sealed tube purged and maintained with an inert atmosphere of nitrogen, were placed 4-{1-methyl-2-oxo-7-azaspiro[3.5]nonan-7-yl}-N-[3-nitro-4-({[(1r,4r)-4-hydroxy-4-methylcyclohexyl]methyl}amino)benzenesulfonyl]-2-[(3R,8S)-15-{[2-(trimethylsilyl)ethoxy]methyl}-2,5-dioxa-9,15,17-triazatetracyclo[8.7.0.0\hat{ }{3,8}.0\hat{ }{12,16}]heptadeca-1(10),11,13,16-tetraen-9-yl]benzamide (200 mg, 0.2 mmol, 1.0 eq), 4-(5-{[(3S)-3-(2-isopropoxyphenyl)piperazin-1-yl]methyl}-3-methoxypyridin-2-yl)morpholine (assumed) (89 mg, 0.2 mmol, 1.0 eq), ZnCl₂ (85 mg, 0.6 mmol, 3.0 eq), NaBH₃CN (39 mg, 0.6 mmol, 3.0 eq), MeOH (2 mL). The resulting solution was stirred for 16 hours at 70° C. The reaction was then quenched by the addition of water (30 mL) and extracted with ethyl acetate (2×30 mL). The combined organic layer was washed with brine (2×30 mL) and dried over anhydrous Na₂SO₄. After filtration, the filtrate was concentrated under vacuum. The crude residue was applied onto a silica gel column and eluted with dichloromethane/methanol=15:1 to give 4-[(1R,2R)-2-[(2S)-2-(2-isopropoxyphenyl)-4-{[5-methoxy-6-(morpholin-4-yl)pyridin-3-yl]methyl}piperazin-1-yl]-1-methyl-7-azaspiro[3.5]nonan-7-yl]-N-[3-nitro-4-({[(1r,4r)-4-hydroxy-4-methylcyclohexyl]methyl}amino)benzenesulfonyl]-2-[(3R,8S)-15-{[2-(trimethylsilyl)ethoxy]methyl}-2,5-dioxa-9,15,17-triazatetracyclo[8.7.0.0\hat{ }{3,8}.0\hat{ }{12,16}]heptadeca-1(10),11,13,16-tetraen-9-yl]benzamide (assumed) as a yellow solid (50 mg, 17.5%) and 4-[(1S,2R)-2-[(2S)-2-(2-isopropoxyphenyl)-4-{[5-methoxy-6-(morpholin-4-yl)pyridin-3-yl]methyl}piperazin-1-yl]-1-methyl-7-azaspiro[3.5]nonan-7-yl]-N-[3-nitro-4-({[(1r,4r)-4-hydroxy-4-methylcyclohexyl]methyl}amino)benzenesulfonyl]-2-[(3R,8S)-15-{[2-(trimethylsilyl)ethoxy]methyl}-2,5-dioxa-9,15,17-triazatetracyclo[8.7.0.0\hat{ }{3,8}.0\hat{ }{12,16}]heptadeca-1(10),11,13,16-tetraen-9-yl]benzamide (assumed) as a yellow solid (50 mg, 17.5%). LC-MS (ESI, m/z) M+1: 1369.

Synthesis of 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-4-[(1S,2R)-2-[(2S)-2-(2-isopropoxyphenyl)-4-{[5-methoxy-6-(morpholin-4-yl)pyridin-3-yl]methyl}piperazin-1-yl]-1-methyl-7-azaspiro[3.5]nonan-7-yl]-N-[3-nitro-4-({[(1r,4r)-4-hydroxy-4-methylcyclohexyl]methyl}amino)benzenesulfonyl]benzamide (assumed): Into an 8 mL vial, were placed 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-4-[(1S,2R)-2-[(2S)-2-(2-isopropoxyphenyl)-4-{[5-methoxy-6-(morpholin-4-yl)pyridin-3-yl]methyl}piperazin-1-yl]-1-methyl-7-azaspiro[3.5]nonan-7-yl]-N-[3-nitro-4-({[(1r,4r)-4-hydroxy-4-methylcyclohexyl]methyl}amino)benzenesulfonyl]benzamide (assumed) (45 mg, 0.04 mmol, 1.0 eq), ethylenediamine (44 mg, 0.7 mmol, 20.0 eq) and TBAF in THE (1 M, 1 mL). The resulting solution was stirred for 6 hours at 80° C. The reaction was then quenched by the addition of water (30 mL) and extracted with ethyl acetate (2×30 mL). The combined organic layer was washed with brine (2×30 mL) and dried over anhydrous Na₂SO₄. After filtration, the filtrate was concentrated under vacuum. The crude residue was applied onto a silica gel column and eluted with dichloromethane/methanol=15:1 to give 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-4-[(1S,2R)-2-[(2S)-2-(2-isopropoxyphenyl)-4-{[5-methoxy-6-(morpholin-4-yl)pyridin-3-yl]methyl}piperazin-1-yl]-1-methyl-7-azaspiro[3.5]nonan-7-yl]-N-[3-nitro-4-({[(1r,4r)-4-hydroxy-4-methylcyclohexyl]methyl}amino)benzenesulfonyl]benzamide (assumed) as a yellow solid (3.6 mg, 8.0%). LC-MS (ESI, m/z) M+1: 1239. ¹H NMR (400 MHz, Methanol-d₄) δ 8.53-8.46 (m, 1H), 7.93-7.86 (m, 1H), 7.71-7.66 (m, 1H), 7.55-7.45 (m, 2H), 7.34-7.28 (m, 1H), 7.25-7.18 (m, 1H), 7.10-7.05 (m, 1H), 7.01-6.88 (m, 3H), 6.74-6.64 (m, 2H), 6.55-6.47 (m, 1H), 6.04-5.99 (m, 1H), 4.65-4.55 (m, 1H), 4.35-4.25 (m, 2H), 3.89-3.84 (m, 1H), 3.83-3.77 (m, 3H), 3.76-3.67 (m, 4H), 3.67-3.54 (m, 4H), 3.53-3.43 (m, 3H), 3.28-3.23 (m, 2H), 3.17-3.07 (m, 1H), 3.03-2.95 (m, 1H), 2.88-2.70 (m, 3H), 2.69-2.60 (m, 1H), 2.56-2.38 (m, 1H), 2.17-1.97 (m, 3H), 1.94-1.66 (m, 10H), 1.65-1.48 (m, 6H), 1.45-1.38 (m, 3H), 1.35-1.27 (m, 12H), 1.26-1.21 (m, 3H).

### Example 121 Preparation of 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-4-[(1R,2R)-2-[(2S)-2-(2-isopropoxyphenyl)-4-{[5-methoxy-6-(morpholin-4-yl)pyridin-3-yl]methyl}piperazin-1-yl]-1-methyl-7-azaspiro[3.5]nonan-7-yl]-N-[3-nitro-4-({[(1r,4r)-4-hydroxy-4-methylcyclohexyl]methyl}amino)benzenesulfonyl]benzamide (assumed)

Synthesis of 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-4-[(1R,2R)-2-[(2S)-2-(2-isopropoxyphenyl)-4-{[5-methoxy-6-(morpholin-4-yl)pyridin-3-yl]methyl}piperazin-1-yl]-1-methyl-7-azaspiro[3.5]nonan-7-yl]-N-[3-nitro-4-({[(1r,4r)-4-hydroxy-4-methylcyclohexyl]methyl}amino)benzenesulfonyl]benzamide (assumed): Into an 8 mL vial, were placed 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-4-[(1R,2R)-2-[(2S)-2-(2-isopropoxyphenyl)-4-{[5-methoxy-6-(morpholin-4-yl)pyridin-3-yl]methyl}piperazin-1-yl]-1-methyl-7-azaspiro[3.5]nonan-7-yl]-N-[3-nitro-4-({[(1r,4r)-4-hydroxy-4-methylcyclohexyl]methyl}amino)benzenesulfonyl]benzamide (assumed) (45 mg, 0.04 mmol, 1.0 eq), ethylenediamine (44 mg, 0.7 mmol, 20.0 eq) and TBAF in THE (1 M, 1 mL). The resulting solution was stirred for 6 hours at 80° C. The reaction was then quenched by the addition of water (30 mL) and extracted with ethyl acetate (2×30 mL). The combined organic layer was washed with brine (2×30 mL) and dried over anhydrous Na₂SO₄. After filtration, the filtrate was concentrated under vacuum. The crude residue was applied onto a silica gel column and eluted with dichloromethane/methanol=15:1 to give 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-4-[(1R,2R)-2-[(2S)-2-(2-isopropoxyphenyl)-4-{[5-methoxy-6-(morpholin-4-yl)pyridin-3-yl]methyl}piperazin-1-yl]-1-methyl-7-azaspiro[3.5]nonan-7-yl]-N-[3-nitro-4-({[(1r,4r)-4-hydroxy-4-methylcyclohexyl]methyl}amino)benzenesulfonyl]benzamide (assumed) as a yellow solid. (2.1 mg, 4.7%). LC-MS (ESI, m/z) M+1: 1239. ¹H NMR (400 MHz, Methanol-d₄) δ 8.53-8.46 (m, 1H), 7.91-7.84 (m, 1H), 7.73-7.67 (m, 1H), 7.51-7.45 (m, 1H), 7.45-7.40 (m, 1H), 7.33-7.29 (m, 1H), 7.25-7.18 (m, 1H), 7.10-7.06 (m, 1H), 6.97-6.86 (m, 3H), 6.72-6.63 (m, 2H), 6.55-6.47 (m, 1H), 6.04-5.94 (m, 1H), 4.65-4.55 (m, 1H), 4.35-4.25 (m, 2H), 3.89-3.84 (m, 1H), 3.83-3.77 (m, 3H), 3.76-3.67 (m, 4H), 3.67-3.54 (m, 4H), 3.53-3.43 (m, 3H), 3.28-3.23 (m, 2H), 3.17-3.07 (m, 1H), 3.03-2.95 (m, 1H), 2.88-2.70 (m, 3H), 2.69-2.60 (m, 1H), 2.56-2.38 (m, 1H), 2.17-1.97 (m, 3H), 1.94-1.66 (m, 10H), 1.65-1.48 (m, 6H), 1.45-1.38 (m, 3H), 1.35-1.27 (m, 12H), 1.26-1.21 (m, 3H).

### Example 122 Preparation of 2-[(3R,8S)-13-fluoro-2,5-dioxa-9,15,17-triazatetracyclo[8.7.0.0\hat{ }{3,8}.0\hat{ }{12,16}]heptadeca-1(10),11,13,16-tetraen-9-yl]-4-{2-[(2S)-2-(2-isopropoxyphenyl)-4-{[5-methoxy-6-(morpholin-4-yl)pyridin-3-yl]methyl}piperazin-1-yl]-7-azaspiro[3.5]nonan-7-yl}-N-[3-nitro-4-({[(1r,4r)-4-hydroxy-4-methylcyclohexyl]methyl}amino)benzenesulfonyl]benzamide (assumed)

Synthesis of 2-[(3R,8S)-13-fluoro-15-{[2-(trimethylsilyl)ethoxy]methyl}-2,5-dioxa-9,15,17-triazatetracyclo[8.7.0.0\hat{ }{3,8}.0\hat{ }{12,16}]heptadeca-1(10),11,13,16-tetraen-9-yl]-4-{2-[(2S)-2-(2-isopropoxyphenyl)-4-{[5-methoxy-6-(morpholin-4-yl)pyridin-3-yl]methyl}piperazin-1-yl]-7-azaspiro[3.5]nonan-7-yl}-N-[3-nitro-4-({[(1r,4r)-4-hydroxy-4-methylcyclohexyl]methyl}amino)benzenesulfonyl]benzamide (assumed): Into an 8-mL sealed tube purged and maintained with an inert atmosphere of nitrogen, were placed 2-[(3R,8S)-13-fluoro-15-{[2-(trimethylsilyl)ethoxy]methyl}-2,5-dioxa-9,15,17-triazatetracyclo[8.7.0.0\hat{ }{3,8}.0\hat{ }{12,16}]heptadeca-1(10),11,13,16-tetraen-9-yl]-N-[3-nitro-4-({[(1r,4r)-4-hydroxy-4-methylcyclohexyl]methyl}amino)benzenesulfonyl]-4-{2-oxo-7-azaspiro[3.5]nonan-7-yl}benzamide (100 mg, 0.1 mmol, 1.0 eq), 4-(5-{[(3S)-3-(2-isopropoxyphenyl)piperazin-1-yl]methyl}-3-methoxypyridin-2-yl)morpholine (assumed) (44 mg, 0.1 mmol, 1.0 eq), ZnCl₂ (42 mg, 0.3 mmol, 3.0 eq), NaBH₃CN (20 mg, 0.3 mmol, 3.0 eq), MeOH (1 mL). The resulting solution was stirred for 5 hours at 70° C. The reaction was then quenched by the addition of water (30 mL) and extracted with ethyl acetate (2×30 mL). The combined organic layer was washed with brine (2×30 mL) and dried over anhydrous Na₂SO₄. After filtration, the filtrate was concentrated under vacuum. The crude residue was applied onto a silica gel column and eluted with dichloromethane/methanol=10:1 to give 2-[(3R,8S)-13-fluoro-15-{[2-(trimethylsilyl)ethoxy]methyl}-2,5-dioxa-9,15,17-triazatetracyclo[8.7.0.0\hat{ }{3,8}.0\hat{ }{12,16}]heptadeca-1(10),11,13,16-tetraen-9-yl]-4-{2-[(2S)-2-(2-isopropoxyphenyl)-4-{[5-methoxy-6-(morpholin-4-yl)pyridin-3-yl]methyl}piperazin-1-yl]-7-azaspiro[3.5]nonan-7-yl}-N-[3-nitro-4-({[(1r,4r)-4-hydroxy-4-methylcyclohexyl]methyl}amino)benzenesulfonyl]benzamide (assumed) as a yellow solid (60 mg, 42.1%). LC-MS (ESI, m/z) M+1: 1373.

Synthesis of 2-[(3R,8S)-13-fluoro-2,5-dioxa-9,15,17-triazatetracyclo[8.7.0.0\hat{ }{3,8}.0\hat{ }{12,16}]heptadeca-1(10),11,13,16-tetraen-9-yl]-4-{2-[(2S)-2-(2-isopropoxyphenyl)-4-{[5-methoxy-6-(morpholin-4-yl)pyridin-3-yl]methyl}piperazin-1-yl]-7-azaspiro[3.5]nonan-7-yl}-N-[3-nitro-4-({[(1r,4r)-4-hydroxy-4-methylcyclohexyl]methyl}amino)benzenesulfonyl]benzamide (assumed): Into an 8-mL sealed tube, were placed 2-[(3R,8S)-13-fluoro-15-{[2-(trimethylsilyl)ethoxy]methyl}-2,5-dioxa-9,15,17-triazatetracyclo[8.7.0.0\hat{ }{3,8}.0\hat{ }{12,16}]heptadeca-1(10),11,13,16-tetraen-9-yl]-4-{2-[(2S)-2-(2-isopropoxyphenyl)-4-{[5-methoxy-6-(morpholin-4-yl)pyridin-3-yl]methyl}piperazin-1-yl]-7-azaspiro[3.5]nonan-7-yl}-N-[3-nitro-4-({[(1r,4r)-4-hydroxy-4-methylcyclohexyl]methyl}amino)benzenesulfonyl]benzamide (assumed) (60 mg, 0.1 mmol, 1.0 eq), ethylenediamine (53 mg, 0.9 mmol, 20.0 eq) and TBAF in THF(1 M, 1 mL). The resulting solution was stirred for 2 hours at 50° C. The reaction was then quenched by the addition of water (30 mL) and extracted with ethyl acetate (2×30 mL). The combined organic layer was washed with brine (2×30 mL) and dried over anhydrous Na₂SO₄. After filtration, the filtrate was concentrated under vacuum. The crude residue was applied onto a silica gel column and eluted with dichloromethane/methanol=15:1 to give 2-[(3R,8S)-13-fluoro-2,5-dioxa-9,15,17-triazatetracyclo[8.7.0.0\hat{ }{3,8}.0\hat{ }{12,16}]heptadeca-1(10),11,13,16-tetraen-9-yl]-4-{2-[(2S)-2-(2-isopropoxyphenyl)-4-{[5-methoxy-6-(morpholin-4-yl)pyridin-3-yl]methyl}piperazin-1-yl]-7-azaspiro[3.5]nonan-7-yl}-N-[3-nitro-4-({[(1r,4r)-4-hydroxy-4-methylcyclohexyl]methyl}amino)benzenesulfonyl]benzamide (assumed) as a yellow solid (3.0 mg, 5.5%). LC-MS (ESI, m/z) M+1: 1243. ¹H NMR (400 MHz, Methanol-d₄) δ 8.48 (d, J=2.3 Hz, 1H), 7.84-7.78 (m, 1H), 7.73-7.68 (m, 1H), 7.71-7.65 (m, 1H), 7.63-7.56 (m, 1H), 7.51-7.42 (m, 2H), 7.00-6.86 (m, 3H), 6.87-6.78 (m, 1H), 6.77-6.62 (m, 2H), 6.40-6.34 (m, 1H), 4.69-4.58 (m, 1H), 4.23 (t, J=9.3 Hz, 3H), 3.89-3.87 (m, 3H), 3.80 (t, J=4.6 Hz, 4H), 3.72-3.65 (m, 1H), 3.58-3.56 (m, 2H), 3.50-3.44 (m, 2H), 3.29-3.26 (m, 2H), 3.19-3.08 (m, 6H), 3.02-2.96 (m, 1H), 2.51-2.39 (m, 1H), 2.30-2.17 (m, 2H), 2.09-1.92 (m, 2H), 1.90-1.82 (m, 2H), 1.81-1.68 (m, 5H), 1.65-1.40 (m, 10H), 1.40-1.23 (m, 15H).

### Example 123 Preparation of N-((4-((((1r,4r)-4-hydroxy-4-methylcyclohexyl)methyl)amino)-3-nitrophenyl)sulfonyl)-4-(2-((R)-4-((5-methoxy-6-morpholinopyridin-3-yl)methyl)-2-(o-tolyl)piperazin-1-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide (assumed)

Synthesis of 4-(3-methoxy-5-{[(3R)-3-(2-methylphenyl)piperazin-1-yl]methyl}pyridin-2-yl)morpholine (assumed): Into an 8 mL vial were added (2R)-2-(2-methylphenyl)piperazine (70 mg, 0.4 mmol, 1.0 eq), 5-methoxy-6-(morpholin-4-yl)pyridine-3-carbaldehyde (88 mg, 0.4 mmol, 1.0 eq), DCE (5 mL), NaBH(OAc)₃ (252 mg, 1.2 mmol, 3.0 eq). The resulting mixture was stirred for 4 hours at 25° C. The reaction was quenched with water (10 mL) and then extracted with dichloromethane (3×10 mL). The combined organic layer was dried over anhydrous Na₂SO₄. After filtration, the filtrate was concentrated under vacuum. The residue was purified by Prep-TLC (dichloromethane/methanol=10:1) to give 4-(3-methoxy-5-{[(3R)-3-(2-methylphenyl)piperazin-1-yl]methyl}pyridin-2-yl)morpholine as a colorless oil (70 mg, 46.0%). LC-MS (ESI, m/z) M+1: 383.

Synthesis of N-((4-((((1r,4r)-4-hydroxy-4-methylcyclohexyl)methyl)amino)-3-nitrophenyl)sulfonyl)-4-(2-((R)-4-((5-methoxy-6-morpholinopyridin-3-yl)methyl)-2-(o-tolyl)piperazin-1-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide (assumed): Into an 8 mL vial, were added 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-N-[3-nitro-4-({[(1r,4r)-4-hydroxy- 4-methylcyclohexyl]methyl}amino)benzenesulfonyl]-4-{2-oxo-7-azaspiro[3.5]nonan-7-yl}benzamide (70 mg, 0.08 mmol, 1.0 eq), 4-(3-methoxy-5-{[(3R)-3-(2-methylphenyl)piperazin-1-yl]methyl}pyridin-2-yl)morpholine (33 mg, 0.08 mmol, 1.0 eq), MeOH (4 mL), ZnCl₂ (35 mg, 0.24 mmol, 3.0 eq), NaBH₃CN (16 mg, 0.24 mmol, 3.0 eq). The resulting mixture was stirred for 7 hours at 70° C. The reaction was quenched with water (5 mL) and extracted with dichloromethane (3×10 mL). The combined organic layer was dried over anhydrous Na₂SO₄. After filtration, the filtrate was concentrated under vacuum. The residue was purified by Prep-TLC (dichloromethane/methanol=16:1) to give N-((4-((((1r,4r)-4-hydroxy-4-methylcyclohexyl)methyl)amino)-3-nitrophenyl)sulfonyl)-4-(2-((R)-4-((5-methoxy-6-morpholinopyridin-3-yl)methyl)-2-(o-tolyl)piperazin-1-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide as a yellow solid (15 mg, 14.7%). LC-MS (ESI, m/z) M+1: 1180. ¹H NMR (400 MHz, Methanol-d₄) δ 8.46 (dd, J=6.6, 2.6 Hz, 2H), 7.86 (d, J=9.3 Hz, 1H), 7.67 (d, J=1.7 Hz, 1H), 7.46 (dd, J=9.3, 2.2 Hz, 1H), 7.26 (d, J=1.7 Hz, 1H), 7.11 (q, J=5.3, 3.9 Hz, 3H), 7.05 (d, J=3.5 Hz, 1H), 6.95-6.87 (m, 1H), 6.66 (d, J=9.7 Hz, 2H), 6.46 (s, 1H), 6.00 (d, J=3.5 Hz, 1H), 4.27 (d, J=7.5 Hz, 2H), 3.85 (s, 1H), 3.78 (t, J=4.8 Hz, 7H), 3.65 (d, J=13.4 Hz, 2H), 3.59-3.40 (m, 4H), 3.24 (t, J=6.2 Hz, 2H), 3.17-2.90 (m, 8H), 2.67-2.64 (m, 3H), 2.33 (s, 6H), 1.82 (d, J=13.8 Hz, 3H), 1.71 (d, J=12.1 Hz, 5H), 1.59-1.08 (m, 13H), 0.99-0.83 (m, 2H).

### Example 124 Preparation of N-((4-((((1r,4r)-4-hydroxy-4-methylcyclohexyl)methyl)amino)-3-nitrophenyl)sulfonyl)-4-(2-((R)-4-((5-(methoxy-d3)-6-morpholinopyridin-3-yl)methyl)-2-(o-tolyl)piperazin-1-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide (assumed)

Synthesis of (R)-4-(3-(methoxy-d3)-5-((3-(o-tolyl)piperazin-1-yl)methyl)pyridin-2-yl)morpholine (assumed): Into a 40-mL sealed-tube, were placed (R)-2-(o-tolyl)piperazine (assumed) (150 mg, 0.5 mmol, 1.0 eq), 5-(methoxy-d3)-6-morpholinonicotinaldehyde (118 mg, 0.5 mmol, 1.1 eq), DCE (4 mL), NaBH(OAc)₃ (307 mg, 1.4 mmol, 3.0 eq). The resulting solution was stirred for 2 hours at 25° C. The reaction was then quenched by the addition of water (40 mL) and extracted with ethyl acetate (2×40 mL). The combined organic layer was washed with brine (2×40 mL) and dried over anhydrous Na₂SO₄. After filtration, the filtrate was concentrated under vacuum. The crude residue was purified onto a silica gel column and eluted with ethyl acetate/petroleum ether=1:2 to give (R)-4-(3-(methoxy-d3)-5-((3-(o-tolyl)piperazin-1-yl)methyl)pyridin-2-yl)morpholine as a light yellow solid (100 mg, 40.1%). LC-MS (ESI, m/z) M+1: 386.

Synthesis of N-((4-((((1r,4r)-4-hydroxy-4-methylcyclohexyl)methyl)amino)-3-nitrophenyl)sulfonyl)-4-(2-((R)-4-((5-(methoxy-d3)-6-morpholinopyridin-3-yl)methyl)-2-(o-tolyl)piperazin-1-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide (assumed): Into an 8 mL vial, were added N-((4-((((1r,4r)-4-hydroxy-4-methylcyclohexyl)methyl)amino)-3-nitrophenyl)sulfonyl)-4-(2-oxo-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide (87 mg, 0.1 mmol, 0.7 eq), (R)-4-(3-(methoxy-d3)-5-((3-(o-tolyl)piperazin-1-yl)methyl)pyridin-2-yl)morpholine (assumed) (45 mg, 0.1 mmol, 1.0 eq), NaBH₃CN (41 mg, 0.7 mmol, 5.0 eq), ZnCl₂ (90 mg, 0.7 mmol, 5.0 eq) and MeOH (2 mL). The resulting mixture was stirred for 5 hours at 70° C. The reaction was quenched by the addition of water (2 mL) and extracted with CH₂Cl₂ (3×3 mL). The combined organic layer was washed with brine (3 mL), and dried over anhydrous Na₂SO₄. After filtration, the filtrate was concentrated under vacuum. The residue was purified by Prep-TLC (CH₂Cl₂/MeOH=20:1) to give N-((4-((((1r,4r)-4-hydroxy-4-methylcyclohexyl)methyl)amino)-3-nitrophenyl)sulfonyl)-4-(2-((R)-4-((5-(methoxy-d3)-6-morpholinopyridin-3-yl)methyl)-2-(o-tolyl)piperazin-1-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide (assumed) as a yellow solid (18 mg, 18.9%). LC-MS (ESI, m/z) M+1: 1184. ¹H NMR (300 MHz, Chloroform-d) δ 12.58 (s, 1H), 8.65 (d, J=2.2 Hz, 1H), 8.45 (d, J=6.0 Hz, 2H), 8.11 (d, J=9.3 Hz, 1H), 7.81 (d, J=10.2 Hz, 1H), 7.17 (s, 2H), 7.10 (s, 1H), 6.84 (d, J=8.7 Hz, 1H), 6.71 (d, J=9.3 Hz, 1H), 6.63 (s, 1H), 6.43 (s, 1H), 6.11 (s, 1H), 4.61-4.39 (m, 1H), 3.98 (d, J=11.7 Hz, 1H), 3.86 (s, 4H), 3.55 (d, J=11.4 Hz, 1H), 3.48 (d, J=10.2 Hz, 4H), 3.39 (s, 4H), 3.21 (t, J=6.3 Hz, 2H), 3.07 (s, 4H), 2.38 (s, 3H), 1.88-1.72 (m, 4H), 1.28 (s, 6H), 1.23 (d, J=12.3 Hz, 12H), 0.89 (d, J=7.5 Hz, 6H), 0.09 (s, 8H).

### Example 125 Preparation of 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-4-{2-[(2R)-4-{2H,3H-[1,4]dioxino[2,3-b]pyridin-7-ylmethyl}-2-(2-methylphenyl)piperazin-1-yl]-7-azaspiro[3.5]nonan-7-yl}-N-[3-nitro-4-({[(1r,4r)-4-hydroxy-4-methylcyclohexyl]methyl}amino)benzenesulfonyl]benzamide (assumed)

Synthesis of (3R)-1-{2H,3H-[1,4]dioxino[2,3-b]pyridin-7-ylmethyl}-3-(2-methylphenyl)piperazine (assumed): Into an 8 mL vial were added 2H,3H-[1,4]dioxino[2,3-b]pyridine-7-carbaldehyde (60 mg, 0.4 mmol, 1.0 eq), (2R)-2-(2-methylphenyl)piperazine (6 mg, 0.4 mmol, 1.0 eq), NaBH₃CN (114 mg, 1.8 mmol, 5.0 eq), ZnCl₂ (248 mg, 1.8 mmol, 5.0 eq) and MeOH (1 mL). The resulting mixture was stirred for 2 hours at 25° C. The crude residue was purified by Prep-TLC (CH₂Cl₂/MeOH=10:1) to give (3R)-1-{2H,3H-[1,4]dioxino[2,3-b]pyridin-7-ylmethyl}-3-(2-methylphenyl)piperazine as a white solid (28 mg, 23.7%). ¹H NMR (300 MHz, Chloroform-d) δ 7.73 (d, J=2.0 Hz, 1H), 7.57-7.49 (m, 1H), 7.26-7.20 (m, 1H), 7.16 (tq, J=6.4, 3.9, 3.1 Hz, 1H), 4.47-4.38 (m, 3H), 4.27 (ddq, J=8.3, 4.1, 2.2 Hz, 3H), 4.19-4.08 (m, 1H), 3.48 (d, J=2.0 Hz, 2H), 3.10 (td, J=10.6, 5.2 Hz, 2H), 2.94-2.83 (m, 2H), 2.35 (s, 3H), 2.27 (td, J=10.8, 4.0 Hz, 1H), 2.13-2.03 (m, 1H).

Synthesis of 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-4-{2-[(2R)-4-{2H,3H-[1,4]dioxino[2,3-b]pyridin-7-ylmethyl}-2-(2-methylphenyl)piperazin-1-yl]-7-azaspiro[3.5]nonan-7-yl}-N-[3-nitro-4-({[(1r,4r)-4-hydroxy-4-methylcyclohexyl]methyl}amino)benzenesulfonyl]benzamide (assumed): Into an 8 mL vial were added (3R)-1-{2H,3H-[1,4]dioxino[2,3-b]pyridin-7-ylmethyl}-3-(2-methylphenyl)piperazine (28 mg, 0.1 mmol, 1.0 eq), 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-N-[3-nitro-4-({[(1r,4r)-4-hydroxy-4-methylcyclohexyl]methyl}amino)benzenesulfonyl]-4-{2-oxo-7-azaspiro[3.5]nonan-7-yl}benzamide (70 mg, 0.1 mmol, 1.0 eq), NaBH₃CN (27 mg, 0.4 mmol, 5.0 eq), ZnCl₂ (59 mg, 0.4 mmol, 5.0 eq) and MeOH (2 mL). The resulting mixture was stirred for 2 hours at 70° C. The reaction was quenched by the addition of water (2 mL) and extracted with CH₂Cl₂ (3×5 mL). The combined organic layer was washed with brine (5 mL), dried over anhydrous Na₂SO₄. After filtration, the filtrate was concentrated under vacuum. The residue was purified by Prep-TLC (CH₂Cl₂/MeOH=17:1) to give 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-4-{2-[(2R)-4-{2H,3H-[1,4]dioxino[2,3-b]pyridin-7-ylmethyl}-2-(2-methylphenyl)piperazin-1-yl]-7-azaspiro[3.5]nonan-7-yl}-N-[3-nitro-4-({[(1r,4r)-4-hydroxy-4-methylcyclohexyl]methyl}amino)benzenesulfonyl]benzamide as a yellow solid (7 mg, 7.2%). LC-MS (ESI, m/z) M+1: 1124. ¹H NMR (300 MHz, Chloroform-d) δ 12.58 (s, 1H), 8.65 (s, 1H), 8.46 (s, 1H), 8.38 (s, 1H), 8.11 (d, J=9.4 Hz, 1H), 7.78 (dd, J=19.6, 8.2 Hz, 2H), 7.21-7.06 (m, 3H), 6.98 (s, 1H), 6.84 (d, J=8.9 Hz, 1H), 6.71 (d, J=9.2 Hz, 1H), 6.64 (s, 1H), 6.43 (s, 1H), 6.11 (s, 1H), 4.71 (s, 1H), 4.60-4.39 (m, 3H), 4.25 (s, 2H), 4.05-3.88 (m, 2H), 3.67 (s, 2H), 3.60-3.37 (m, 4H), 3.21 (t, J=6.0 Hz, 2H), 3.06 (s, 4H), 2.43-2.21 (m, 4H), 1.86 (dd, J=17.1, 11.8 Hz, 8H), 1.44 (d, J=18.5 Hz, 6H), 1.37-1.16 (m, 14H).

### Example 126 Preparation of N-((4-((((1r,4r)-4-hydroxy-4-methylcyclohexyl)methyl)amino)-3-nitrophenyl)sulfonyl)-4-(2-((S)-2-(2-isopropoxyphenyl)-4-((5-methoxy-6-((S)-3-methylmorpholino)pyridin-3-yl)methyl)piperazin-1-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide (assumed)

Synthesis of (S)-4-(5-(((S)-3-(2-isopropoxyphenyl)piperazin-1-yl)methyl)-3-methoxypyridin-2-yl)-3-methylmorpholine (assumed): Into an 8 mL sealed tube were added (S)-5-methoxy-6-(3-methylmorpholino)nicotinaldehyde (129 mg, 0.5 mmol, 0.8 eq), (2S)-2-(2-isopropoxyphenyl)piperazine (assumed) (150 mg, 0.7 mmol, 1.0 eq), NaBH₃CN (128 mg, 2.0 mmol, 3.0 eq), MeOH (3 mL) and ZnCl₂ (278 mg, 2.0 mmol, 3.0 eq) at 25° C. The resulting mixture was stirred for 16 hours at 25° C. The reaction was quenched by the addition of water (2 mL), and extracted with CH₂Cl₂ (2×10 mL). The combined organic layers were washed with brine (20 mL), dried over anhydrous Na₂SO₄. After filtration, the filtrate was concentrated under vacuum. The residue was purified by Prep-TLC (dichloromethane/methanol=12:1) to give (S)-4-(5-(((S)-3-(2-isopropoxyphenyl)piperazin-1-yl)methyl)-3-methoxypyridin-2-yl)-3-methylmorpholine (assumed) as a colorless oil (90 mg, 30.0%). LC-MS (ESI, m/z) M+1: 441.

Synthesis of N-((4-((((1r,4r)-4-hydroxy-4-methylcyclohexyl)methyl)amino)-3-nitrophenyl)sulfonyl)-4-(2-((S)-2-(2-isopropoxyphenyl)-4-((5-methoxy-6-((S)-3-methylmorpholino)pyridin-3-yl)methyl)piperazin-1-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide (assumed): Into an 8-mL sealed-tube, were placed (S)-4-(5-(((S)-3-(2-isopropoxyphenyl)piperazin-1-yl)methyl)-3-methoxypyridin-2-yl)-3-methylmorpholine (assumed) (30 mg, 0.07 mmol, 1.0 eq), N-((4-((((1r,4r)-4-hydroxy-4-methylcyclohexyl)methyl)amino)-3-nitrophenyl)sulfonyl)-4-(2-oxo-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide (55 mg, 0.07 mmol, 1.0 eq), MeOH (3 mL), ZnCl₂ (28 mg, 0.2 mmol, 3.0 eq) and NaBH₃CN (13 mg, 0.2 mmol, 3.0 eq). The resulting solution was stirred for 4 hours at 70° C. The resulting mixture was then quenched by the addition of water (20 mL) and extracted with dichloromethane/methanol (10:1, 2×30 mL). The combined organic layers was washed with brine (2×20 mL), dried over anhydrous Na₂SO₄. After filtration, the filtrate was concentrated under vacuum. The residue was purified by Prep-TLC (dichloromethane/methanol=15:1) to give N-((4-((((1r,4r)-4-hydroxy-4-methylcyclohexyl)methyl)amino)-3-nitrophenyl)sulfonyl)-4-(2-((S)-2-(2-isopropoxyphenyl)-4-((5-methoxy-6-((S)-3-methylmorpholino)pyridin-3-yl)methyl)piperazin-1-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide (assumed) as a light yellow solid (25 mg, 29.6%). LC-MS (ESI, m/z) M+1: 1239. ¹H NMR (400 MHz, DMSO-d₆) δ 12.75 (s, 1H), 11.24 (s, 1H), 8.56 (s, 1H), 8.38 (s, 1H), 7.78 (d, J=9.1 Hz, 1H), 7.65 (s, 1H), 7.39 (m, 2H), 7.13 (m, 3H), 6.98 (s, 1H), 6.90-6.70 (m, 4H), 6.44 (s, 1H), 6.05 (s, 1H), 4.53 (s, 1H), 4.23 (s, 2H), 4.06 (m, 2H), 3.77 (m, 4H), 3.70 (d, J=10.6 Hz, 2H), 3.61-3.46 (m, 3H), 3.13 (m, 4H), 3.03 (m, 4H), 2.89 (s, 3H), 2.83 (s, 1H), 2.18 (s, 3H), 1.91 (s, 2H), 1.68 (m, 4H), 1.55 (m, 4H), 1.35 (m, 3H), 1.31 (m, 9H), 1.27-1.18 (m, 3H), 1.11 (d, J=4.3 Hz, 7H), 0.97 (m, 4H).

### Example 127 Preparation of 4-(2-((S)-4-((3,3-dimethyl-2,3-dihydro-[1,4]dioxino[2,3-b]pyridin-8-yl)methyl)-2-(2-isopropoxyphenyl)piperazin-1-yl)-7-azaspiro[3.5]nonan-7-yl)-N-((4-((((1r,4r)-4-hydroxy-4-methylcyclohexyl)methyl)amino)-3-nitrophenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide (assumed)

Synthesis of (S)-8-((3-(2-isopropoxyphenyl)piperazin-1-yl)methyl)-3,3-dimethyl-2,3-dihydro-[1,4]dioxino[2,3-b]pyridine (assumed): Into an 8 mL sealed tube were added 3,3-dimethyl-2H-[1,4]dioxino[2,3-b]pyridine-8-carbaldehyde (105 mg, 0.5 mmol, 0.8 eq), (2S)-2-(2-isopropoxyphenyl)piperazine (assumed) (150 mg, 0.7 mmol, 1.0 eq), NaBH₃CN (128 mg, 2.0 mmol, 3.0 eq), MeOH (3 mL) and ZnCl₂ (278 mg, 2.0 mmol, 3.0 eq) at 25° C. The resulting mixture was stirred for overnight at 25° C. The reaction was quenched by the addition of water (2 mL), and extracted with CH₂Cl₂ (2×10 mL). The combined organic layers were washed with brine (20 mL), dried over anhydrous Na₂SO₄. After filtration, the filtrate was concentrated under vacuum. The residue was purified by Prep-TLC (dichloromethane/methanol=12:1) to give (S)-8-((3-(2-isopropoxyphenyl)piperazin-1-yl)methyl)-3,3-dimethyl-2,3-dihydro-[1,4]dioxino[2,3-b]pyridine (assumed) as a colorless oil (80 mg, 29.6%). LC-MS (ESI, m/z) M+1: 398.

Synthesis of 4-(2-((S)-4-((3,3-dimethyl-2,3-dihydro-[1,4]dioxino[2,3-b]pyridin-8-yl)methyl)-2-(2-isopropoxyphenyl)piperazin-1-yl)-7-azaspiro[3.5]nonan-7-yl)-N-((4-((((1r,4r)-4-hydroxy-4-methylcyclohexyl)methyl)amino)-3-nitrophenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide (assumed): Into an 8-mL sealed-tube, were placed (S)-8-((3-(2-isopropoxyphenyl)piperazin-1-yl)methyl)-3,3-dimethyl-2,3-dihydro-[1,4]dioxino[2,3-b]pyridine (assumed) (30 mg, 0.08 mmol, 1.0 eq), N-((4-((((1r,4r)-4-hydroxy-4-methylcyclohexyl)methyl)amino)-3-nitrophenyl)sulfonyl)-4-(2-oxo-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide (61 mg, 0.08 mmol, 1.0 eq), MeOH (3 mL), ZnCl₂ (31 mg, 0.2 mmol, 3.0 eq) and NaBH₃CN (14 mg, 0.2 mmol, 3.0 eq). The resulting solution was stirred for 4 hours at 70° C. The resulting mixture was then quenched by the addition of water (20 mL), and extracted with dichloromethane/methanol (10:1, 2×30 mL). The combined organics was washed with brine (2×20 mL), dried over anhydrous Na₂SO₄. After filtration, the filtrate was concentrated under vacuum. The residue was purified by Prep-TLC (dichloromethane/methanol=15:1) to give 4-(2-((S)-4-((3,3-dimethyl-2,3-dihydro-[1,4]dioxino[2,3-b]pyridin-8-yl)methyl)-2-(2-isopropoxyphenyl)piperazin-1-yl)-7-azaspiro[3.5]nonan-7-yl)-N-((4-((((1r,4r)-4-hydroxy-4-methylcyclohexyl)methyl)amino)-3-nitrophenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide (assumed) as a light yellow solid (25 mg, 27.7%). LC-MS (ESI, m/z) M+1: 1196. ¹H NMR (400 MHz, DMSO-d₆) δ 12.75 (s, 1H), 11.24 (s, 1H), 8.56 (s, 1H), 8.37 (s, 1H), 7.78 (d, J=9.7 Hz, 1H), 7.68 (d, J=5.0 Hz, 1H), 7.41 (s, 1H), 7.39-7.29 (m, 1H), 7.13 (m, 3H), 6.97-6.88 (m, 1H), 6.87-6.79 (m, 4H), 6.44 (s, 1H), 6.05 (s, 1H), 4.56 (m, 1H), 4.23 (s, 2H), 4.07 (m, 2H), 3.92 (s, 2H), 3.86-3.80 (m, 1H), 3.75 (m, 1H), 3.49 (d, J=8.0 Hz, 1H), 3.13 (s, 3H), 3.04 (s, 3H), 2.88 (d, J=9.5 Hz, 2H), 2.82 (s, 1H), 2.24-2.18 (m, 3H), 2.02 (s, 1H), 1.68 (d, J=13.7 Hz, 4H), 1.60-1.56 (m, 4H), 1.53 (s, 1H), 1.40-1.35 (m, 6H), 1.32-1.16 (m, 15H), 1.16-1.08 (m, 5H), 0.85 (m, 1H).

### Example 128 Preparation of N-((4-((((1r,4r)-4-hydroxy-4-methylcyclohexyl)methyl)amino)-3-nitrophenyl)sulfonyl)-4-(2-((S)-2-(2-isopropoxyphenyl)-4-((5-morpholinopyridin-2-yl)methyl)piperazin-1-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide (assumed)

Synthesis of (S)-4-(6-((3-(2-isopropoxyphenyl)piperazin-1-yl)methyl)pyridin-3-yl)morpholine (assumed): Into a 40-mL sealed-tube, were placed (S)-2-(2-isopropoxyphenyl)piperazine (assumed) (150 mg, 0.5 mmol, 1.0 eq), 5-morpholinopicolinaldehyde (118 mg, 0.5 mmol, 1.1 eq), DCE (4 mL), NaBH(OAc)₃ (307 mg, 1.4 mmol, 3.0 eq). The resulting solution was stirred for 5 hours at 25° C. The resulting mixture was then quenched by the addition of water (40 mL), and extracted with ethyl acetate (2×40 mL). The combined organic layers was washed with brine (2×40 mL), dried over anhydrous Na₂SO₄. After filtration, the filtrate was concentrated under vacuum. The crude residue was purified onto a silica gel column and eluted with ethyl acetate/petroleum ether=1:2 to give (S)-4-(6-((3-(2-isopropoxyphenyl)piperazin-1-yl)methyl)pyridin-3-yl)morpholine (assumed) as a light yellow solid (110 mg, 44.1%). LC-MS (ESI, m/z) M+1: 397.

Synthesis of N-((4-((((1r,4r)-4-hydroxy-4-methylcyclohexyl)methyl)amino)-3-nitrophenyl)sulfonyl)-4-(2-((S)-2-(2-isopropoxyphenyl)-4-((5-morpholinopyridin-2-yl)methyl)piperazin-1-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide (assumed): Into an 8 mL vial, were added N-((4-((((1r,4r)-4-hydroxy-4-methylcyclohexyl)methyl)amino)-3-nitrophenyl)sulfonyl)-4-(2-oxo-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide (87 mg, 0.1 mmol, 0.7 eq), (S)-4-(6-((3-(2-isopropoxyphenyl)piperazin-1-yl)methyl)pyridin-3-yl)morpholine (assumed) (45 mg, 0.1 mmol, 1.0 eq), NaBH₃CN (41 mg, 0.7 mmol, 5.0 eq), ZnCl₂ (90 mg, 0.7 mmol, 5.0 eq) and MeOH (2 mL). The resulting mixture was stirred for 4 hours at 70° C. The reaction was quenched by the addition of water (2 mL), and extracted with CH₂Cl₂ (3×3 mL). The combined organic layers was washed with brine (3 mL), dried over anhydrous Na₂SO₄. After filtration, the filtrate was concentrated under vacuum. The residue was purified by Prep-TLC (CH₂Cl₂/MeOH=20:1) to give N-((4-((((1r,4r)-4-hydroxy-4-methylcyclohexyl)methyl)amino)-3-nitrophenyl)sulfonyl)-4-(2-((S)-2-(2-isopropoxyphenyl)-4-((5-morpholinopyridin-2-yl)methyl)piperazin-1-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide (assumed) as a yellow solid (15 mg, 15.9%). LC-MS (ESI, m/z) M+1: 1194. ¹H NMR (300 MHz, Chloroform-d) δ 12.57 (s, 2H), 8.65 (s, 2H), 8.46 (s, 2H), 8.40 (s, 1H), 8.11 (d, J=9.6 Hz, 2H), 7.80 (s, 2H), 7.07 (d, J=16.5 Hz, 3H), 6.98 (s, 1H), 6.85 (d, J=8.1 Hz, 3H), 6.71 (d, J=9.3 Hz, 2H), 6.62 (s, 1H), 6.43 (s, 2H), 6.11 (s, 3H), 4.61-4.50 (m, 1H), 4.00 (s, 1H), 3.91-3.84 (m, 10H), 3.58 (s, 2H), 3.51 (d, J=9.0 Hz, 3H), 3.36-3.31 (m, 8H), 3.21 (t, J=6.3 Hz, 3H), 3.06 (s, 9H), 1.75 (s, 3H), 1.51 (d, J=12.3 Hz, 1H), 1.37 (dd, J=14.4, 9.0 Hz, 9H), 0.89 (d, J=8.1 Hz, 3H).

### Example 129 Preparation of N-((4-((((1r,4r)-4-hydroxy-4-methylcyclohexyl)methyl)amino)-3-nitrophenyl)sulfonyl)-4-(2-((S)-2-(2-isopropoxyphenyl)-4-((7-methoxy-2-methylbenzofuran-5-yl)methyl)piperazin-1-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide (assumed)

Synthesis of (S)-3-(2-isopropoxyphenyl)-1-((7-methoxy-2-methylbenzofuran-5-yl)methyl)piperazine (assumed): Into a 40-mL sealed-tube, were placed (S)-2-(2-isopropoxyphenyl)piperazine (assumed) (50 mg, 0.5 mmol, 1.0 eq), 7-methoxy-2-methylbenzofuran-5-carbaldehyde (78 mg, 0.5 mmol, 1.1 eq), MeOH (4 mL), NaBH(OAc)₃ (157 mg, 1.4 mmol, 3.0 eq). The resulting solution was stirred for 2 hours at 25° C. The resulting mixture was then quenched by the addition of water (40 mL), and extracted with ethyl acetate (2×40 mL). The combined organic layers was washed with brine (2×40 mL), dried over anhydrous Na₂SO₄. After filtration, the filtrate was concentrated under vacuum. The crude residue was purified onto a silica gel column and eluted with ethyl acetate/petroleum ether=1:2 to give (S)-3-(2-isopropoxyphenyl)-1-((7-methoxy-2-methylbenzofuran-5-yl)methyl)piperazine as a light yellow solid (60 mg, 44.1%). LC-MS (ESI, m/z) M+1: 395.

Synthesis of N-((4-((((1r,4r)-4-hydroxy-4-methylcyclohexyl)methyl)amino)-3-nitrophenyl)sulfonyl)-4-(2-((S)-2-(2-isopropoxyphenyl)-4-((7-methoxy-2-methylbenzofuran-5-yl)methyl)piperazin-1-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide (assumed): Into an 8 mL vial, were added N-((4-((((1r,4r)-4-hydroxy-4-methylcyclohexyl)methyl)amino)-3-nitrophenyl)sulfonyl)-4-(2-oxo-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide (87 mg, 0.1 mmol, 0.7 eq), (S)-3-(2-isopropoxyphenyl)-1-((7-methoxy-2-methylbenzofuran-5-yl)methyl)piperazine (assumed) (45 mg, 0.1 mmol, 1.0 eq), NaBH₃CN (41 mg, 0.7 mmol, 5.0 eq), ZnCl₂ (90 mg, 0.7 mmol, 5.0 eq) and MeOH (2 mL). The resulting mixture was stirred for 5 hours at 70° C. The reaction was quenched by the addition of water (2 mL), and extracted with CH₂Cl₂ (3×3 mL). The combined organic layers was washed with brine (3 mL), dried over anhydrous Na₂SO₄. After filtration, the filtrate was concentrated under vacuum. The residue was purified by Prep-TLC (CH₂Cl₂/MeOH 20:1) to give N-((4-((((1r,4r)-4-hydroxy-4-methylcyclohexyl)methyl)amino)-3-nitrophenyl)sulfonyl)-4-(2-((S)-2-(2-isopropoxyphenyl)-4-((7-methoxy-2-methylbenzofuran-5-yl)methyl)piperazin-1-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide (assumed) as a yellow solid (15 mg, 15.9%). LC-MS (ESI, m/z) M+1: 1193. ¹H NMR (300 MHz, Chloroform-d) δ 12.77 (s, 1H), 8.75 (s, 1H), 8.46 (s, 2H), 8.35 (s, 1H), 8.11 (d, J=9.6 Hz, 3H), 7.80 (s, 2H), 7.07 (d, J=16.5 Hz, 3H), 6.98 (s, 1H), 6.85-6.81 (m, J=8.1 Hz, 3H), 6.71 (d, J=9.3 Hz, 2H), 6.62 (s, 1H), 6.43 (s, 2H), 6.11 (s, 2H), 4.61-4.50 (m, 1H), 4.00 (s, 1H), 3.86-3.88 (m, 9H), 3.58 (s, 1H), 3.51 (d, J=9.0 Hz, 3H), 3.38 (s, 9H), 3.21 (t, J=6.3 Hz, 3H), 3.05-3.01 (m, 11H), 1.75 (s, 2H), 1.51 (d, J=12.3 Hz, 1H), 1.38 (dd, J=14.7, 9.0 Hz, 9H), 0.89 (d, J=8.1 Hz, 3H).

### Example 130 Preparation of 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-4-{2-[(2S)-2-(2-isopropoxyphenyl)-4-{[5-methoxy-6-(morpholin-4-yl)pyridin-3-yl]methyl}piperazin-1-yl]-7-azaspiro[3.5]nonan-7-yl}-N-[(3S)-5-nitro-3-[(1r,4S)-4-hydroxy-4-methylcyclohexyl]-3,4-dihydro-2H-1,4-benzoxazin-7-ylsulfonyl]benzamide (assumed)

Synthesis of 4-{2-[(2S)-2-(2-isopropoxyphenyl)-4-{[5-methoxy-6-(morpholin-4-yl)pyridin-3-yl]methyl}piperazin-1-yl]-7-azaspiro[3.5]nonan-7-yl}-N-[(3S)-5-nitro-3-[(1r,4S)-4-hydroxy-4-methylcyclohexyl]-3,4-dihydro-2H-1,4-benzoxazin-7-ylsulfonyl]-2-[(3R,8S)-15-{[2-(trimethylsilyl)ethoxy]methyl}-2,5-dioxa-9,15,17-triazatetracyclo[8.7.0.0\hat{ }{3,8}.0\hat{ }{12,16}]heptadeca-1(10),11,13,16-tetraen-9-yl]benzamide (assumed): Into an 8 mL vial were added 4-{2-[(2S)-2-(2-isopropoxyphenyl)-4-{[5-methoxy-6-(morpholin-4-yl)pyridin-3-yl]methyl}piperazin-1-yl]-7-azaspiro[3.5]nonan-7-yl}-2-[(3R,8S)-15-{[2-(trimethylsilyl)ethoxy]methyl}-2,5-dioxa-9,15,17-triazatetracyclo[8.7.0.0\hat{ }{3,8}.0\hat{ }{12,16}]heptadeca-1(10),11,13,16-tetraen-9-yl]benzoic acid (assumed) (90 mg, 0.09 mmol, 1.0 eq), (S)-3-((1r,4S)-4-hydroxy-4-methylcyclohexyl)-5-nitro-3,4-dihydro-2H-benzo[b][1,4]oxazine-7-sulfonamide (32 mg, 0.09 mmol, 1.0 eq), EDCl (34 mg, 0.1 mmol, 2.0 eq), DMAP (43 mg, 0.3 mmol, 4.0 eq) and CH₂Cl₂ (2 mL). The resulting mixture was stirred for 2 hours at 25° C. The resulting mixture was diluted with water (5 mL). The resulting mixture was extracted with CH₂Cl₂ (3×10 mL). The combined organic layers were washed with brine (3×10 mL), dried over anhydrous Na₂SO₄. After filtration, the filtrate was concentrated under vacuum. The residue was purified by Prep-TLC (CH₂Cl₂/MeOH 20:1) to give 4-{2-[(2S)-2-(2-isopropoxyphenyl)-4-{[5-methoxy-6-(morpholin-4-yl)pyridin-3-yl]methyl}piperazin-1-yl]-7-azaspiro[3.5]nonan-7-yl}-N-[(3S)-5-nitro-3-[(1r,4S)-4-hydroxy-4-methylcyclohexyl]-3,4-dihydro-2H-1,4-benzoxazin-7-ylsulfonyl]-2-[(3R,8S)-15-{[2-(trimethylsilyl)ethoxy]methyl}-2,5-dioxa-9,15,17-triazatetracyclo[8.7.0.0\hat{ }{3,8}.0\hat{ }{12,16}]heptadeca-1(10),11,13,16-tetraen-9-yl]benzamide (assumed) as a yellow solid (70 mg, 57.9%). LC-MS (ESI, m/z) M+1: 1383.

Synthesis of 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-4-{2-[(2S)-2-(2-isopropoxyphenyl)-4-{[5-methoxy-6-(morpholin-4-yl)pyridin-3-yl]methyl}piperazin-1-yl]-7-azaspiro[3.5]nonan-7-yl}-N-[(3S)-5-nitro-3-[(1r,4S)-4-hydroxy-4-methylcyclohexyl]-3,4-dihydro-2H-1,4-benzoxazin-7-ylsulfonyl]benzamide (assumed): Into an 8 mL vial were added 4-{2-[(2S)-2-(2-isopropoxyphenyl)-4-{[5-methoxy-6-(morpholin-4-yl)pyridin-3-yl]methyl}piperazin-1-yl]-7-azaspiro[3.5]nonan-7-yl}-N-[(3S)-5-nitro-3-[(1r,4S)-4-hydroxy-4-methylcyclohexyl]-3,4-dihydro-2H-1,4-benzoxazin-7-ylsulfonyl]-2-[(3R,8S)-15-{[2-(trimethylsilyl)ethoxy]methyl}-2,5-dioxa-9,15,17-triazatetracyclo[8.7.0.0\hat{ }{3,8}.0\hat{ }{12,16}]heptadeca-1(10),11,13,16-tetraen-9-yl]benzamide (assumed) (65 mg, 0.05 mmol, 1.0 eq), ethylenediamine (56 mg, 0.9 mmol, 20.0 eq), TBAF in THE (246 mg, 0.9 mmol, 20.0 eq). The resulting mixture was stirred for 4 hours at 70° C. The reaction was quenched by the addition of water (2 mL). The resulting mixture was extracted with CH₂Cl₂ (3×10 mL). The combined organic layers were washed with brine (3×10 mL), dried over anhydrous Na₂SO₄. After filtration, the filtrate was concentrated under vacuum. The residue was purified by Prep-TLC (CH₂Cl₂/MeOH 10:1) to give 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-4-{2-[(2S)-2-(2-isopropoxyphenyl)-4-{[5-methoxy-6-(morpholin-4-yl)pyridin-3-yl]methyl}piperazin-1-yl]-7-azaspiro[3.5]nonan-7-yl}-N-[(3S)-5-nitro-3-[(1r,4S)-4-hydroxy-4-methylcyclohexyl]-3,4-dihydro-2H-1,4-benzoxazin-7-ylsulfonyl]benzamide (assumed) as a yellow solid (7 mg, 11.9%). LC-MS (ESI, m/z) M+1: 1254. ¹H NMR (400 MHz, Chloroform-d) δ 12.70 (s, 1H), 12.60 (d, J=6.2 Hz, 1H), 8.45 (s, 1H), 8.34 (s, 1H), 8.30 (d, J=2.2 Hz, 1H), 8.13 (d, J=9.2 Hz, 1H), 7.81 (s, 1H), 7.44 (s, 1H), 7.09 (s, 1H), 7.05 (d, J=7.5 Hz, 1H), 6.99 (s, 1H), 6.84 (t, J=8.7 Hz, 2H), 6.68 (d, J=8.4 Hz, 1H), 6.43 (s, 1H), 6.14 (s, 1H), 4.75-4.57 (m, 2H), 4.57-4.38 (m, 3H), 4.18 (dd, J=11.3, 3.2 Hz, 1H), 4.11-3.92 (m, 3H), 3.85 (d, J=4.8 Hz, 6H), 3.58 (s, 3H), 3.54-3.33 (m, 8H), 3.08 (d, J=13.7 Hz, 6H), 2.70 (d, J=11.0 Hz, 3H), 2.29-1.98 (m, 4H), 1.98-1.76 (m, 5H), 1.50-1.40 (m, 4H), 1.37 (d, J=6.0 Hz, 6H), 1.30 (d, J=11.1 Hz, 9H).

### Example 131 Preparation of 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-4-{2-[(2S)-2-(2-isopropoxyphenyl)-4-{[5-methoxy-6-(morpholin-4-yl)pyridin-3-yl]methyl}piperazin-1-yl]-7-azaspiro[3.5]nonan-7-yl}-N-{3-nitro-4-[(oxan-4-ylmethyl)amino]benzenesulfonyl}benzamide (assumed)

Synthesis of 4-{2-[(2S)-2-(2-isopropoxyphenyl)-4-{[5-methoxy-6-(morpholin-4-yl)pyridin-3-yl]methyl}piperazin-1-yl]-7-azaspiro[3.5]nonan-7-yl}-N-{3-nitro-4-[(oxan-4-ylmethyl)amino]benzenesulfonyl}-2-[(3R,8S)-15-{[2-(trimethylsilyl)ethoxy]methyl}-2,5-dioxa-9,15,17-triazatetracyclo[8.7.0.0\hat{ }{3,8}.0\hat{ }{12,16}]heptadeca-1(10),11,13,16-tetraen-9-yl]benzamide (assumed): Into an 8 mL vial were added 4-{2-[(2S)-2-(2-isopropoxyphenyl)-4-{[5-methoxy-6-(morpholin-4-yl)pyridin-3-yl]methyl}piperazin-1-yl]-7-azaspiro[3.5]nonan-7-yl}-2-[(3R,8S)-15-{[2-(trimethylsilyl)ethoxy]methyl}-2,5-dioxa-9,15,17-triazatetracyclo[8.7.0.0 {3,8}.0\hat{ }{12,16}]heptadeca-1(10),11,13,16-tetraen-9-yl]benzoic acid (assumed) (90 mg, 0.09 mmol, 1.0 eq), 3-nitro-4-[(oxan-4-ylmethyl)amino]benzenesulfonamide (28 mg, 0.09 mmol, 1.0 eq), EDCl (34 mg, 0.1 mmol, 2.0 eq), DMAP (43 mg, 0.3 mmol, 4.0 eq) and CH₂Cl₂ (2 mL). The resulting mixture was stirred for 2 hours at 25° C. The resulting mixture was diluted with water (5 mL), and extracted with CH₂Cl₂ (3×10 mL). The combined organic layers were washed with brine (3×10 mL), dried over anhydrous Na₂SO₄. After filtration, the filtrate was concentrated under vacuum. The crude residue was purified by Prep-TLC (CH₂Cl₂/MeOH=20:1) to give 4-{2-[(2S)-2-(2-isopropoxyphenyl)-4-{[5-methoxy-6-(morpholin-4-yl)pyridin-3-yl]methyl}piperazin-1-yl]-7-azaspiro[3.5]nonan-7-yl}-N-{3-nitro-4-[(oxan-4-ylmethyl)amino]benzenesulfonyl}-2-[(3R,8S)-15-{[2-(trimethylsilyl)ethoxy]methyl}-2,5-dioxa-9,15,17-triazatetracyclo[8.7.0.0\hat{ }{3,8}.0\hat{ }{12,16}]heptadeca-1(10),11,13,16-tetraen-9-yl]benzamide (assumed) as a yellow solid (60 mg, 51.7%). LC-MS (ESI, m/z) M+1: 1327.

Synthesis of 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-4-{2-[(2S)-2-(2-isopropoxyphenyl)-4-{[5-methoxy-6-(morpholin-4-yl)pyridin-3-yl]methyl}piperazin-1-yl]-7-azaspiro[3.5]nonan-7-yl}-N-{3-nitro-4-[(oxan-4-ylmethyl)amino]benzenesulfonyl}benzamide (assumed): Into an 8 mL vial were added 4-{2-[(2S)-2-(2-isopropoxyphenyl)-4-{[5-methoxy-6-(morpholin-4-yl)pyridin-3-yl]methyl}piperazin-1-yl]-7-azaspiro[3.5]nonan-7-yl}-N-{3-nitro-4-[(oxan-4-ylmethyl)amino]benzenesulfonyl}-2-[(3R,8S)-15-{[2-(trimethylsilyl)ethoxy]methyl}-2,5-dioxa-9,15,17-triazatetracyclo[8.7.0.0\hat{ }{3,8}.0\hat{ }{12,16}]heptadeca-1(10),11,13,16-tetraen-9-yl]benzamide (assumed) (60 mg, 0.04 mmol, 1.0 eq), ethylenediamine (109 mg, 1.8 mmol, 40.0 eq) and TBAF in THE (1 M, 2 mL). The resulting mixture was stirred for 4 hours at 70° C. The reaction was quenched by the addition of water (2 mL), and extracted with CH₂Cl₂ (3×5 mL). The combined organic layers were washed with brine (3 mL), dried over anhydrous Na₂SO₄. After filtration, the filtrate was concentrated under vacuum. The crude residue was dissolved in EtOAc (10 mL), and then washed with water (5×10 mL), dried over anhydrous Na₂SO₄. After filtration, the filtrate was concentrated under vacuum. The residue was purified by Prep-TLC (CH₂Cl₂/MeOH=17:1) to give 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-4-{2-[(2S)-2-(2-isopropoxyphenyl)-4-{[5-methoxy-6-(morpholin-4-yl)pyridin-3-yl]methyl}piperazin-1-yl]-7-azaspiro[3.5]nonan-7-yl}-N-{3-nitro-4-[(oxan-4-ylmethyl)amino]benzenesulfonyl}benzamide (assumed) as a yellow solid (15 mg, 27.7%). LC-MS (ESI, m/z) M+1: 1197. ¹H NMR (300 MHz, Chloroform-d) δ 12.58 (d, J=4.8 Hz, 1H), 8.65 (s, 1H), 8.46 (s, 1H), 8.35 (s, 1H), 8.11 (d, J=9.1 Hz, 1H), 7.82 (d, J=9.4 Hz, 1H), 7.09 (s, 1H), 7.01 (d, J=18.2 Hz, 1H), 6.84 (t, J=7.9 Hz, 1H), 6.71 (d, J=9.2 Hz, 2H), 6.63 (d, J=6.7 Hz, 1H), 6.42 (s, 2H), 6.11 (s, 1H), 4.74-4.37 (m, 4H), 4.02 (t, J=14.5 Hz, 3H), 3.85 (s, 4H), 3.71-3.31 (m, 8H), 3.31-2.96 (m, 6H), 2.92-2.50 (m, 5H), 2.38 (s, 7H), 1.74 (d, J=13.2 Hz, 4H), 1.43 (d, J=8.7 Hz, 10H), 1.41-1.19 (m, 9H), 0.89 (d, J=7.3 Hz, 5H).

### Example 132 Preparation of 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-4-{2-[(2S)-2-(2-isopropoxyphenyl)-4-{[5-methoxy-6-(morpholin-4-yl)pyridin-3-yl]methyl}piperazin-1-yl]-7-azaspiro[3.5]nonan-7-yl}-N-{3-nitro-4-[(oxan-4-ylmethyl)amino]benzenesulfonyl}benzamide (assumed)

Synthesis of 4-{2-[(2S)-2-(2-isopropoxyphenyl)-4-{[5-methoxy-6-(morpholin-4-yl)pyridin-3-yl]methyl}piperazin-1-yl]-7-azaspiro[3.5]nonan-7-yl}-N-[(3S)-5-nitro-3-(oxan-4-yl)-3,4-dihydro-2H-1,4-benzoxazin-7-ylsulfonyl]-2-[(3R,8S)-15-{[2-(trimethylsilyl)ethoxy]methyl}-2,5-dioxa-9,15,17-triazatetracyclo[8.7.0.0\hat{ }{3,8}.0\hat{ }{12,16}]heptadeca-1(10),11,13,16-tetraen-9-yl]benzamide (assumed): Into an 8 mL vial were added 4-{2-[(2S)-2-(2-isopropoxyphenyl)-4-{[5-methoxy-6-(morpholin-4-yl)pyridin-3-yl]methyl}piperazin-1-yl]-7-azaspiro[3.5]nonan-7-yl}-2-[(3R,8S)-15-{[2-(trimethylsilyl)ethoxy]methyl}-2,5-dioxa-9,15,17-triazatetracyclo[8.7.0.0\hat{ }{3,8}.0\hat{ }{12,16}]heptadeca-1(10),11,13,16-tetraen-9-yl]benzoic acid (assumed) (100 mg, 0.1 mmol, 1.0 eq), (3S)-5-nitro-3-(oxan-4-yl)-3,4-dihydro-2H-1,4-benzoxazine-7-sulfonamide (assumed) (33 mg, 0.1 mmol, 1.0 eq), EDCl (74 mg, 0.4 mmol, 4.0 eq), DMAP (24 mg, 0.1 mmol, 2.0 eq) and CH₂Cl₂ (2 mL). The resulting mixture was stirred for 2 hours at 25° C. The resulting mixture was diluted with water (5 mL), and extracted with CH₂Cl₂ (3×10 mL). The combined organic layers were washed with brine (3×10 mL), dried over anhydrous Na₂SO₄. After filtration, the filtrate was concentrated under vacuum. The residue was purified by Prep-TLC (CH₂Cl₂/MeOH=20:1) to give 4-{2-[(2S)-2-(2-isopropoxyphenyl)-4-{[5-methoxy-6-(morpholin-4-yl)pyridin-3-yl]methyl}piperazin-1-yl]-7-azaspiro[3.5]nonan-7-yl}-N-[(3S)-5-nitro-3-(oxan-4-yl)-3,4-dihydro-2H-1,4-benzoxazin-7-ylsulfonyl]-2-[(3R,8S)-15-{[2-(trimethylsilyl)ethoxy]methyl}-2,5-dioxa-9,15,17-triazatetracyclo[8.7.0.0\hat{ }{3,8}.0\hat{ }{12,16}]heptadeca-1(10),11,13,16-tetraen-9-yl]benzamide (assumed) as a yellow solid (68 mg, 51.7%). LC-MS (ESI, m/z) M+1: 1355.

Synthesis of 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-4-{2-[(2S)-2-(2-isopropoxyphenyl)-4-{[5-methoxy-6-(morpholin-4-yl)pyridin-3-yl]methyl}piperazin-1-yl]-7-azaspiro[3.5]nonan-7-yl}-N-{3-nitro-4-[(oxan-4-ylmethyl)amino]benzenesulfonyl}benzamide (assumed): Into an 8 mL vial were added 4-{2-[(2S)-2-(2-isopropoxyphenyl)-4-{[5-methoxy-6-(morpholin-4-yl)pyridin-3-yl]methyl}piperazin-1-yl]-7-azaspiro[3.5]nonan-7-yl}-N-[(3S)-5-nitro-3-(oxan-4-yl)-3,4-dihydro-2H-1,4-benzoxazin-7-ylsulfonyl]-2-[(3R,8S)-15-{[2-(trimethylsilyl)ethoxy]methyl}-2,5-dioxa-9,15,17-triazatetracyclo[8.7.0.0\hat{ }{3,8}.0\hat{ }{12,16}]heptadeca-1(10),11,13,16-tetraen-9-yl]benzamide (assumed) (50 mg, 0.04 mmol, 1.0 eq), ethylenediamine (88 mg, 1.5 mmol, 40.0 eq), and TBAF in THE (3 M, 2 mL). The resulting mixture was stirred for 4 hours at 70° C. The reaction was quenched by the addition of water (2 mL), and extracted with CH₂Cl₂ (3×5 mL). The combined organic layers were washed with brine (3 mL), dried over anhydrous Na₂SO₄. After filtration, the filtrate was concentrated under vacuum. The residue was dissolved in EtOAc (10 mL), and then washed with 5×10 mL of EtOAc, dried over anhydrous Na₂SO₄. After filtration, the filtrate was concentrated under vacuum. The residue was purified by Prep-TLC (CH₂Cl₂/MeOH=17:1) to give 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-4-{2-[(2S)-2-(2-isopropoxyphenyl)-4-{[5-methoxy-6-(morpholin-4-yl)pyridin-3-yl]methyl}piperazin-1-yl]-7-azaspiro[3.5]nonan-7-yl}-N-{3-nitro-4-[(oxan-4-ylmethyl)amino]benzenesulfonyl}benzamide (assumed) as a yellow solid (8 mg, 17.7%). LC-MS (ESI, m/z) M+1: 1225. ¹H NMR (300 MHz, Chloroform-d) δ 12.62 (d, J=4.4 Hz, 1H), 8.43 (d, J=11.1 Hz, 2H), 8.29 (s, 1H), 8.13 (d, J=9.0 Hz, 1H), 7.80 (s, 1H), 7.71 (d, J=18.0 Hz, 1H), 7.46 (d, J=2.2 Hz, 1H), 7.06 (d, J=14.5 Hz, 1H), 7.00 (d, J=9.0 Hz, 1H), 6.85 (d, J=8.7 Hz, 2H), 6.67 (d, J=6.3 Hz, 1H), 6.43 (s, 1H), 6.13 (s, 1H), 4.79-4.42 (m, 4H), 4.33-3.93 (m, 5H), 3.85 (s, 5H), 3.64-3.31 (m, 9H), 3.08 (d, J=17.7 Hz, 7H), 2.82-2.48 (m, 3H), 2.48-1.93 (m, 5H), 1.63 (d, J=95.3 Hz, 9H), 1.45-1.11 (m, 8H), 1.17-0.76 (m, 6H).

### Example 133 Preparation of 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-4-{2-[(2S)-2-(2-isopropoxyphenyl)-4-{[5-methoxy-6-(morpholin-4-yl)pyridin-3-yl]methyl}piperazin-1-yl]-7-azaspiro[3.5]nonan-7-yl}-N-{3-nitro-4-[(oxan-4-ylmethyl)amino]benzenesulfonyl}benzamide (assumed)

Synthesis of 4-{2-[(2S)-2-(2-isopropoxyphenyl)-4-{[5-methoxy-6-(morpholin-4-yl)pyridin-3-yl]methyl}piperazin-1-yl]-7-azaspiro[3.5]nonan-7-yl}-N-[(3R)-5-nitro-3-(oxan-4-yl)-3,4-dihydro-2H-1,4-benzoxazin-7-ylsulfonyl]-2-[(3R,8S)-15-{[2-(trimethylsilyl)ethoxy]methyl}-2,5-dioxa-9,15,17-triazatetracyclo[8.7.0.0\hat{ }{3,8}.0\hat{ }{12,16}]heptadeca-1(10),11,13,16-tetraen-9-yl]benzamide (assumed): Into an 8 mL vial were added 4-{2-[(2S)-2-(2-isopropoxyphenyl)-4-{[5-methoxy-6-(morpholin-4-yl)pyridin-3-yl]methyl}piperazin-1-yl]-7-azaspiro[3.5]nonan-7-yl}-2-[(3R,8S)-15-{[2-(trimethylsilyl)ethoxy]methyl}-2,5-dioxa-9,15,17-triazatetracyclo[8.7.0.0\hat{ }{3,8}.0\hat{ }{12,16}]heptadeca-1(10),11,13,16-tetraen-9-yl]benzoic acid (assumed) (100 mg, 0.1 mmol, 1.0 eq), (3R)-5-nitro-3-(oxan-4-yl)-3,4-dihydro-2H-1,4-benzoxazine-7-sulfonamide (assumed) (33 mg, 0.1 mmol, 1.0 eq), EDCl (37 mg, 0.2 mmol, 2.0 eq), DMAP (47 mg, 0.4 mmol, 4.0 eq) and CH₂Cl₂ (2 mL). The resulting mixture was stirred for 2 hours at 25° C. The resulting mixture was diluted with water (5 mL) and extracted with CH₂Cl₂ (3×10 mL). The combined organic layers were washed with brine (3×10 mL), dried over anhydrous Na₂SO₄. After filtration, the filtrate was concentrated under vacuum. The residue was purified by Prep-TLC (CH₂Cl₂/MeOH=20:1) to give 4-{2-[(2S)-2-(2-isopropoxyphenyl)-4-{[5-methoxy-6-(morpholin-4-yl)pyridin-3-yl]methyl}piperazin-1-yl]-7-azaspiro[3.5]nonan-7-yl}-N-[(3R)-5-nitro-3-(oxan-4-yl)-3,4-dihydro-2H-1,4-benzoxazin-7-ylsulfonyl]-2-[(3R,8S)-15-{[2-(trimethylsilyl)ethoxy]methyl}-2,5-dioxa-9,15,17-triazatetracyclo[8.7.0.0\hat{ }{3,8}.0\hat{ }{12,16}]heptadeca-1(10),11,13,16-tetraen-9-yl]benzamide (assumed) as a yellow solid (60 mg, 45.6%). LC-MS (ESI, m/z) M+1: 1355.

Synthesis of 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-4-{2-[(2S)-2-(2-isopropoxyphenyl)-4-{[5-methoxy-6-(morpholin-4-yl)pyridin-3-yl]methyl}piperazin-1-yl]-7-azaspiro[3.5]nonan-7-yl}-N-{3-nitro-4-[(oxan-4-ylmethyl)amino]benzenesulfonyl}benzamide (assumed): Into an 8 mL vial were added 4-{2-[(2S)-2-(2-isopropoxyphenyl)-4-{[5-methoxy-6-(morpholin-4-yl)pyridin-3-yl]methyl}piperazin-1-yl]-7-azaspiro[3.5]nonan-7-yl}—N-[(3R)-5-nitro-3-(oxan-4-yl)-3,4-dihydro-2H-1,4-benzoxazin-7-ylsulfonyl]-2-[(3R,8S)-15-{[2-(trimethylsilyl)ethoxy]methyl}-2,5-dioxa-9,15,17-triazatetracyclo[8.7.0.0\hat{ }{3,8}.0\hat{ }{12,16}]heptadeca-1(10),11,13,16-tetraen-9-yl]benzamide (assumed) (50 mg, 0.04 mmol, 1.0 eq), ethylenediamine (89 mg, 1.5 mmol, 40.0 eq) and TBAF in THF(5 M, 2 mL). The resulting mixture was stirred for 4 hours at 70° C. The reaction was quenched by the addition of water (2 mL), and extracted with CH₂Cl₂ (3×5 mL). The combined organic layers were washed with brine (3 mL), dried over anhydrous Na₂SO₄. After filtration, the filtrate was concentrated under vacuum. The residue was dissolved in EtOAc (10 mL). The resulting mixture was washed with EtOAc (5×10 mL), dried over anhydrous Na₂SO₄. After filtration, the filtrate was concentrated under vacuum. The residue was purified by Prep-TLC (CH₂Cl₂/MeOH=7:1) to give 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-4-{2-[(2S)-2-(2-isopropoxyphenyl)-4-{[5-methoxy-6-(morpholin-4-yl)pyridin-3-yl]methyl}piperazin-1-yl]-7-azaspiro[3.5]nonan-7-yl}-N-{3-nitro-4-[(oxan-4-ylmethyl)amino]benzenesulfonyl}benzamide (assumed) as a yellow solid (8 mg, 15.5%). LC-MS (ESI, m/z) M+1: 1225. ¹H NMR (300 MHz, Chloroform-d) δ 12.60 (s, 1H), 8.45 (s, 1H), 8.32 (d, J=8.2 Hz, 1H), 8.13 (d, J=9.0 Hz, 1H), 7.80 (s, 1H), 7.46 (s, 1H), 7.07 (d, J=15.5 Hz, 2H), 6.98 (s, 1H), 6.85 (d, J=7.8 Hz, 3H), 6.68 (d, J=5.8 Hz, 1H), 6.43 (s, 1H), 6.13 (s, 1H), 4.54 (d, J=18.4 Hz, 6H), 4.19-3.91 (m, 4H), 3.85 (s, 5H), 3.58 (s, 2H), 3.55-3.29 (m, 11H), 3.05 (s, 5H), 2.31 (d, J=14.4 Hz, 7H), 1.45 (s, 3H), 1.41-1.32 (m, 6H), 1.29 (d, J=8.0 Hz, 7H), 1.04-0.78 (m, 4H).

### Example 134 Preparation of 4-{2-[(2S)-4-[(6-cyclopropyl-5-methoxypyridin-3-yl)methyl]-2-(2-isopropoxyphenyl)piperazin-1-yl]-7-azaspiro[3.5]nonan-7-yl}-2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-N-[3-nitro-4-({[(1r,4r)-4-hydroxy-4-methylcyclohexyl]methyl}amino)benzenesulfonyl]benzamide (assumed)

Synthesis of (3S)-1-[(6-cyclopropyl-5-methoxypyridin-3-yl)methyl]-3-(2-isopropoxyphenyl)piperazine (assumed): Into an 8 mL vial, were added 6-cyclopropyl-5-methoxypyridine-3-carbaldehyde (80 mg, 0.4 mmol, 1.0 eq), DCE (5 mL), (2S)-2-(2-isopropoxyphenyl)piperazine (assumed) (99 mg, 0.4 mmol, 1.0 eq), NaBH(OAc)₃ (287 mg, 1.2 mmol, 3.0 eq). The resulting mixture was stirred for 4 hours at 25° C. The reaction was quenched with water (10 mL) and extracted with dichloromethane (3×10 mL). The combined organic layers were dried over anhydrous Na₂SO₄. After filtration, the filtrate was concentrated under vacuum. The residue was purified by Prep-TLC (dichloromethane/methanol=10:1) to give (3S)-1-[(6-cyclopropyl-5-methoxypyridin-3-yl)methyl]-3-(2-isopropoxyphenyl)piperazine (assumed) as a colorless oil (60 mg, 34.8%). LC-MS (ESI, m/z) M+1: 382.

Synthesis of 4-{2-[(2S)-4-[(6-cyclopropyl-5-methoxypyridin-3-yl)methyl]-2-(2-isopropoxyphenyl)piperazin-1-yl]-7-azaspiro[3.5]nonan-7-yl}-2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-N-[3-nitro-4-({[(1r,4r)-4-hydroxy-4-methylcyclohexyl]methyl}amino)benzenesulfonyl]benzamide (assumed): Into an 8 mL vial, were added (3S)-1-[(6-cyclopropyl-5-methoxypyridin-3-yl)methyl]-3-(2-isopropoxyphenyl)piperazine (assumed) (16 mg, 0.04 mmol, 1.0 eq), MeOH (2 mL), 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-N-[3-nitro-4-({[(1r,4r)-4-hydroxy- 4-methylcyclohexyl]methyl}amino)benzenesulfonyl]-4-{2-oxo-7-azaspiro[3.5]nonan-7-yl}benzamide (35 mg, 0.04 mmol, 1.0 eq), ZnCl₂ (17 mg, 0.12 mmol, 3.0 eq) and NaBH₃CN (8 mg, 0.12 mmol, 3.0 eq). The resulting mixture was stirred for 4 hours at 70° C. The reaction was quenched by the addition of water (10 mL) and extracted with dichloromethane (3×10 mL). The combined organic layers were dried over anhydrous Na₂SO₄. After filtration, the filtrate was concentrated under vacuum. The residue was purified by Prep-TLC (dichloromethane/methanol=10:1) to give 4-{2-[(2S)-4-[(6-cyclopropyl-5-methoxypyridin-3-yl)methyl]-2-(2-isopropoxyphenyl)piperazin-1-yl]-7-azaspiro[3.5]nonan-7-yl}-2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-N-[3-nitro-4-({[(1r,4r)-4-hydroxy-4-methylcyclohexyl]methyl}amino)benzenesulfonyl]benzamide (assumed) as a yellow solid (13 mg, 25.0%). LC-MS (ESI, m/z) M+1: 1180. ¹H NMR (400 MHz, Methanol-d₄) δ 8.48 (d, J=2.2 Hz, 1H), 7.86 (s, 2H), 7.56-7.40 (m, 2H), 7.32 (s, 1H), 7.08 (d, J=3.5 Hz, 1H), 7.27-7.19 (m, 1H), 7.00-6.86 (m, 3H), 6.68 (d, J=9.7 Hz, 2H), 6.49 (s, 1H), 6.02 (d, J=3.5 Hz, 1H), 4.61 (s, 1H), 4.28 (s, 2H), 4.09-4.18 (m, 1H), 3.90 (d, J=2.6 Hz, 5H), 3.66 (s, 1H), 3.48 (s, 1H), 3.28-3.23 (m, 4H), 3.21-3.02 (s, 7H), 3.01-2.88 (m, 1H), 2.73-2.61 (m, 1H), 2.54-2.29 (m, 4H), 1.89-1.80 (m, 2H), 1.77-1.63 (s, 6H), 1.61-1.36 (m, 8H), 1.36-1.21 (m, 8H), 0.94 (d, J=7.5 Hz, 7H).

### Example 135 Preparation of 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-4-{2-[(2S)-2-(2-isopropoxyphenyl)-4-[(5-methoxy-6-methylpyridin-3-yl)methyl]piperazin-1-yl]-7-azaspiro[3.5]nonan-7-yl}-N-[3-nitro-4-({[(1r,4r)-4-hydroxy-4-methylcyclohexyl]methyl}amino)benzenesulfonyl]benzamide (assumed)

Synthesis of (3S)-3-(2-isopropoxyphenyl)-1-[(5-methoxy-6-methylpyridin-3-yl)methyl]piperazine (assumed): Into an 8 mL vial, were added 5-methoxy-6-methylpyridine-3-carbaldehyde (50 mg, 0.3 mmol, 1.0 eq), DCE (3 mL), (2S)-2-(2-isopropoxyphenyl)piperazine (assumed) (73 mg, 0.3 mmol, 1.0 eq), NaBH(OAc)₃ (210 mg, 1.0 mmol, 3.3 eq). The resulting mixture was stirred for 4 hours at 25° C. The reaction was quenched with water (10 mL) and extracted with dichloromethane (3×10 mL). The combined organic layers were dried over anhydrous Na₂SO₄. After filtration, the filtrate was concentrated under vacuum. The residue was purified by Prep-TLC (dichloromethane/methanol=10:1) to give (3S)-3-(2-isopropoxyphenyl)-1-[(5-methoxy-6-methylpyridin-3-yl)methyl]piperazine (assumed) as a colorless oil (50 mg, 42.5%). LC-MS (ESI, m/z) M+1: 356.

Synthesis of 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-4-{2-[(2S)-2-(2-isopropoxyphenyl)-4-[(5-methoxy-6-methylpyridin-3-yl)methyl]piperazin-1-yl]-7-azaspiro[3.5]nonan-7-yl}-N-[3-nitro-4-({[(1r,4r)-4-hydroxy-4-methylcyclohexyl]methyl}amino)benzenesulfonyl]benzamide (assumed): Into an 8 mL vial, were added (3S)-3-(2-isopropoxyphenyl)-1-[(5-methoxy-6-methylpyridin-3-yl)methyl]piperazine (assumed) (15 mg, 0.04 mmol, 1.0 eq), MeOH (2 mL), 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-N-[3-nitro-4-({[(1r,4r)-4-hydroxy-4-methylcyclohexyl]methyl}amino)benzenesulfonyl]-4-{2-oxo-7-azaspiro[3.5]nonan-7-yl}benzamide (35 mg, 0.04 mmol, 1.0 eq), ZnCl₂ (17 mg, 0.12 mmol, 3.0 eq), NaBH₃CN (8 mg, 0.12 mmol, 3.0 eq). The resulting mixture was stirred for 4 hours at 70° C. The reaction was quenched by the addition of water (3 mL) and extracted with dichloromethane (3×10 mL). The combined organic layers were dried over anhydrous Na₂SO₄. After filtration, the filtrate was concentrated under vacuum. The residue was purified by Prep-TLC (dichloromethane/methanol=10:1) to give 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-4-{2-[(2S)-2-(2-isopropoxyphenyl)-4-[(5-methoxy-6-methylpyridin-3-yl)methyl]piperazin-1-yl]-7-azaspiro[3.5]nonan-7-yl}-N-[3-nitro-4-({[(1r,4r)-4-hydroxy-4-methylcyclohexyl]methyl}amino)benzenesulfonyl]benzamide as a yellow solid (16 mg, 28.5%). LC-MS (ESI, m/z) M+1: 1154. ¹H NMR (400 MHz, Methanol-d₄) δ 8.48 (d, J=2.6 Hz, 1H), 7.98-7.83 (m, 2H), 7.52-7.42 (m, 2H), 7.39 (s, 1H), 7.22 (t, J=7.5 Hz, 1H), 7.07 (d, J=3.5 Hz, 1H), 7.00-6.87 (m, 3H), 6.72-6.64 (m, 2H), 6.49 (s, 1H), 6.02 (d, J=3.5 Hz, 1H), 4.62 (d, J=10.3 Hz, 2H), 4.33-4.02 (m, 3H), 3.89 (d, J=3.9 Hz, 4H), 3.70 (s, 1H), 3.59 (s, 2H), 3.55-3.43 (m, 2H), 3.26 (d, J=6.2 Hz, 3H), 3.22-2.90 (m, 8H), 2.66 (d, J=11.6 Hz, 1H), 2.40 (d, J=2.6 Hz, 5H), 1.95 (s, 1H), 1.84 (d, J=13.0 Hz, 2H), 1.73 (d, J=12.1 Hz, 6H), 1.61-1.47 (m, 6H), 1.42 (d, J=6.2 Hz, 2H), 1.34-1.21 (m, 8H), 0.92 (s, 1H).

### Example 136 Preparation of 4-{2-[(2S)-4-[(5-cyclopropyl-6-methoxypyridin-3-yl)methyl]-2-(2-isopropoxyphenyl)piperazin-1-yl]-7-azaspiro[3.5]nonan-7-yl}-2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-N-[3-nitro-4-({[(1r,4r)-4-hydroxy-4-methylcyclohexyl]methyl}amino)benzenesulfonyl]benzamide (assumed)

Synthesis of (3S)-1-[(5-cyclopropyl-6-methoxypyridin-3-yl)methyl]-3-(2-isopropoxyphenyl)piperazine (assumed): Into a 20-mL sealed-tube, were placed 5-cyclopropyl-6-methoxypyridine-3-carbaldehyde (72 mg, 0.4 mmol, 0.9 eq), (2S)-2-(2-isopropoxyphenyl)piperazine (assumed) (100 mg, 0.4 mmol, 1.0 eq), DCE (2 mL), NaBH(OAc)₃ (289 mg, 1.4 mmol, 3.0 eq). The resulting solution was stirred for 2 hours at 25° C. The resulting mixture was then quenched by the addition of water (20 mL), and extracted with dichloromethane/methanol (20:1, 2×40 mL). The combined organic layers was washed with brine (2×40 mL), dried over anhydrous Na₂SO₄. After filtration, the filtrate was concentrated under vacuum. The crude residue was purified onto a silica gel column and eluted with dichloromethane/methanol=10:1 to give (3S)-1-[(5-cyclopropyl-6-methoxypyridin-3-yl)methyl]-3-(2-isopropoxyphenyl)piperazine (assumed) as a light yellow oil (120 mg, 69.3%). LC-MS (ESI, m/z) M+1: 382.

Synthesis of 4-{2-[(2S)-4-[(5-cyclopropyl-6-methoxypyridin-3-yl)methyl]-2-(2-isopropoxyphenyl)piperazin-1-yl]-7-azaspiro[3.5]nonan-7-yl}-2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-N-[3-nitro-4-({[(1r,4r)-4-hydroxy-4-methylcyclohexyl]methyl}amino)benzenesulfonyl]benzamide (assumed): Into an 8-mL sealed-tube, were placed (3S)-1-[(5-cyclopropyl-6-methoxypyridin-3-yl)methyl]-3-(2-isopropoxyphenyl)piperazine (assumed) (23 mg, 0.05 mmol, 1.2 eq), 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-N-[3-nitro-4-({[(1r,4r)-4-hydroxy- 4-methylcyclohexyl]methyl}amino)benzenesulfonyl]-4-{2-oxo-7-azaspiro[3.5]nonan-7-yl}benzamide (40 mg, 0.05 mmol, 1.0 eq), MeOH (1 mL), ZnCl₂ (20 mg, 0.1 mmol, 3.0 eq), NaBH₃CN (9 mg, 0.1 mmol, 3.0 eq). The resulting solution was stirred for 3 hours at 70° C. The resulting mixture was then quenched by the addition of water (20 mL), and extracted with dichloromethane/methanol (10:1, 2×30 mL). The combined organic layers was washed with brine (2×20 mL), dried over anhydrous Na₂SO₄. After filtration, the filtrate was concentrated under vacuum. The residue was purified by Prep-TLC (dichloromethane/methanol=15:1) to give 4-{2-[(2S)-4-[(5-cyclopropyl-6-methoxypyridin-3-yl)methyl]-2-(2-isopropoxyphenyl)piperazin-1-yl]-7-azaspiro[3.5]nonan-7-yl}-2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-N-[3-nitro-4-({[(1r,4r)-4-hydroxy-4-methylcyclohexyl]methyl}amino)benzenesulfonyl]benzamide (assumed) as a light yellow solid (10 mg, 17.3%). LC-MS (ESI, m/z) M+1: 1180. ¹H NMR (400 MHz, Methanol-d₄) δ 8.46 (d, J=2.4 Hz, 1H), 7.87-7.76 (m, 2H), 7.52-7.37 (m, 2H), 7.25-7.17 (m, 2H), 7.05 (d, J=3.4 Hz, 1H), 7.01-6.84 (m, 3H), 6.69-6.61 (m, 2H), 6.47 (s, 1H), 5.99 (d, J=3.6 Hz, 1H), 4.67-4.54 (m, 1H), 4.26 (d, J=7.2 Hz, 1H), 4.12-4.05 (m, 1H), 3.91 (s, 3H), 3.89-3.86 (m, 1H), 3.67 (br, 1H), 3.51-3.42 (m, 4H), 3.25-3.22 (m, 2H), 3.17-3.05 (m, 6H), 2.92 (d, J=9.8 Hz, 1H), 2.63-2.59 (m, 1H), 2.42-2.18 (m, 3H), 2.06-1.99 (m. 2H), 1.91 (br, 1H), 1.82 (d, J=13.2 Hz, 1H), 1.72-1.63 (m, 4H), 1.54-1.46 (m, 5H), 1.41-1.33 (m, 3H), 1.30-1.27 (m, 7H), 1.25-1.22 (m, 7H), 0.94-0.88 (m, 2H), 0.66-0.62 (m, 2H).

### Example 137 Preparation of 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-4-{2-[(2S)-4-{[5-isopropoxy-6-(morpholin-4-yl)pyridin-3-yl]methyl}-2-(2-isopropoxyphenyl)piperazin-1-yl]-7-azaspiro[3.5]nonan-7-yl}-N-[3-nitro-4-({[(1r,4r)-4-hydroxy-4-methylcyclohexyl]methyl}amino)benzenesulfonyl]benzamide (assumed)

Synthesis of 4-(3-isopropoxy-5-{[(3S)-3-(2-isopropoxyphenyl)piperazin-1-yl]methyl}pyridin-2-yl)morpholine (assumed): Into an 8-mL sealed tube purged and maintained with an inert atmosphere of nitrogen, were placed 5-isopropoxy-6-(morpholin-4-yl)pyridine-3-carbaldehyde (114 mg, 0.5 mmol, 1.0 eq), (2S)-2-(2-isopropoxyphenyl)piperazine (assumed) (100 mg, 0.5 mmol, 1.0 eq), NaBH(OAc)₃ (289 mg, 1.4 mmol, 3.0 eq), DCE (1 mL). The resulting solution was stirred for 2 hours at 25° C. The resulting mixture was then quenched by the addition of water (30 mL), and extracted with dichloromethane (2×30 mL). The combined organic layers was washed with brine (2×30 mL), dried over anhydrous Na₂SO₄. After filtration, the filtrate was concentrated under vacuum. The crude residue was applied onto a silica gel column and eluted with dichloromethane/methanol=10:1 to give 4-(3-isopropoxy-5-{[(3S)-3-(2-isopropoxyphenyl)piperazin-1-yl]methyl}pyridin-2-yl)morpholine (assumed) as a light yellow solid (130 mg, 63.0%). LC-MS (ESI, m/z) M+1: 455.

Synthesis of 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-4-{2-[(2S)-4-{[5-isopropoxy-6-(morpholin-4-yl)pyridin-3-yl]methyl}-2-(2-isopropoxyphenyl)piperazin-1-yl]-7-azaspiro[3.5]nonan-7-yl}-N-[3-nitro-4-({[(1r,4r)-4-hydroxy-4-methylcyclohexyl]methyl}amino)benzenesulfonyl]benzamide (assumed): Into an 8-mL sealed tube, were placed 4-(3-isopropoxy-5-{[(3S)-3-(2-isopropoxyphenyl)piperazin-1-yl]methyl}pyridin-2-yl)morpholine (assumed) (22 mg, 0.05 mmol, 1.0 eq), 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-N-[3-nitro-4-({[(1r,4r)-4-hydroxy-4-methylcyclohexyl]methyl}amino)benzenesulfonyl]-4-{2-oxo-7-azaspiro[3.5]nonan-7-yl}benzamide (40 mg, 0.05 mmol, 1.0 eq), ZnCl₂ (20 mg, 0.1 mmol, 3.0 eq), NaBH₃CN (9 mg, 0.1 mmol, 3.0 eq), MeOH (0.5 mL). The resulting solution was stirred for 4 hours at 70° C. The resulting mixture was then quenched by the addition of water (30 mL), and extracted with dichloromethane (2×30 mL). The combined organic layers was washed with brine (2×30 mL), dried over anhydrous Na₂SO₄. After filtration, the filtrate was concentrated under vacuum. The crude residue was applied onto a silica gel column and eluted with dichloromethane/methanol=15:1 to give 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-4-{2-[(2S)-4-{[5-isopropoxy-6-(morpholin-4-yl)pyridin-3-yl]methyl}-2-(2-isopropoxyphenyl)piperazin-1-yl]-7-azaspiro[3.5]nonan-7-yl}-N-[3-nitro-4-({[(1r,4r)-4-hydroxy-4-methylcyclohexyl]methyl}amino)benzenesulfonyl]benzamide (assumed) as a yellow solid (10 mg, 16.3%). LC-MS (ESI, m/z) M+1: 1209. ¹H NMR (400 MHz, Methanol-d₄) δ 8.52-8.45 (m, 1H), 7.92-7.61 (m, 2H), 7.51-7.41 (m, 2H), 7.31-7.19 (m, 2H), 7.10-6.86 (m, 4H), 6.70 (s, 2H), 6.55-6.46 (m, 1H), 6.05-5.94 (m, 1H), 4.70-4.58 (m, 2H), 4.35-4.21 (m, 2H), 3.97-3.88 (m, 1H), 3.83-3.77 (m, 4H), 3.75-3.63 (m, 1H), 3.60-3.45 (m, 2H), 3.28-3.23 (m, 2H), 3.22-3.04 (m, 5H), 3.01-2.92 (m, 1H), 2.71-2.62 (m, 1H), 2.52-2.22 (m, 3H), 1.90-1.79 (m, 2H), 1.78-1.64 (m, 5H), 1.63-1.47 (m, 4H), 1.46-1.38 (m, 4H), 1.37-1.29 (m, 16H), 1.28-1.23 (m, 9H), 0.95-0.85 (m, 2H).

### Example 138 Preparation of N-((4-((((1r,4r)-4-hydroxy-4-methylcyclohexyl)methyl)amino)-3-nitrophenyl)sulfonyl)-4-(2-((R)-4-((5-methoxy-6-((S)-3-methylmorpholino)pyridin-3-yl)methyl)-2-(o-tolyl)piperazin-1-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide

Synthesis of (S)-4-(3-methoxy-5-(((R)-3-(o-tolyl)piperazin-1-yl)methyl)pyridin-2-yl)-3-methylmorpholine: Into an 8 mL sealed tube were added 5-methoxy-6-[(3S)-3-methylmorpholin-4-yl]pyridine-3-carbaldehyde (150 mg, 0.6 mmol, 0.8 eq), (2R)-2-(2-methylphenyl)piperazine (140 mg, 0.8 mmol, 1.0 eq), NaBH₃CN (150 mg, 2.4 mmol, 3.0 eq), MeOH (3 mL) and ZnCl₂ (325 mg, 2.4 mmol, 3.0 eq) at 25° C. The resulting mixture was stirred for 16 hours at 25° C. The reaction was quenched by the addition of water (2 mL), and extracted with CH₂Cl₂ (2×10 mL). The combined organic layers were washed with brine (20 mL), dried over anhydrous Na₂SO₄. After filtration, the filtrate was concentrated under vacuum. The residue was purified by Prep-TLC (dichloromethane/methanol=12:1) to give (S)-4-(3-methoxy-5-(((R)-3-(o-tolyl)piperazin-1-yl)methyl)pyridin-2-yl)-3-methylmorpholine as a colorless oil (80 mg, 25.4%). LC-MS (ESI, m/z) M+1: 397.

Synthesis of N-((4-((((1r,4r)-4-hydroxy-4-methylcyclohexyl)methyl)amino)-3-nitrophenyl)sulfonyl)-4-(2-((R)-4-((5-methoxy-6-((S)-3-methylmorpholino)pyridin-3-yl)methyl)-2-(o-tolyl)piperazin-1-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide: Into an 8-mL sealed-tube, were placed (3S)-4-(3-methoxy-5-{[(3R)-3-(2-methylphenyl)piperazin-1-yl]methyl}pyridin-2-yl)-3-methylmorpholine (30 mg, 0.08 mmol, 1.0 eq), N-((4-((((1r,4r)-4-hydroxy-4-methylcyclohexyl)methyl)amino)-3-nitrophenyl)sulfonyl)-4-(2-oxo-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide (62 mg, 0.08 mmol, 1.0 eq), MeOH (3 mL), ZnCl₂ (31 mg, 0.2 mmol, 3.0 eq) and NaBH₃CN (14 mg, 0.2 mmol, 3.0 eq). The resulting solution was stirred for 4 hours at 70° C. The resulting mixture was then quenched by the addition of water (20 mL), and extracted with dichloromethane/methanol (10:1, 2×30 mL). The combined organic layers was washed with brine (2×20 mL), dried over anhydrous Na₂SO₄. After filtration, the filtrate was concentrated under vacuum. The residue was purified by Prep-TLC (dichloromethane/methanol=15:1) to give N-((4-((((1r,4r)-4-hydroxy-4-methylcyclohexyl)methyl)amino)-3-nitrophenyl)sulfonyl)-4-(2-((R)-4-((5-methoxy-6-((S)-3-methylmorpholino)pyridin-3-yl)methyl)-2-(o-tolyl)piperazin-1-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide as a light yellow solid (25 mg, 27.7%). LC-MS (ESI, m/z) M+1: 1195. ¹H NMR (400 MHz, DMSO-d₆) δ 12.75 (s, 1H), 11.23 (s, 1H), 8.56 (s, 1H), 8.37 (s, 1H), 7.77 (s, 1H), 7.67 (s, 1H), 7.40 (s, 2H), 7.15-7.12 (m, 3H), 7.08 (s, 2H), 6.97 (s, 1H), 6.84-6.80 (m, 2H), 6.43 (s, 1H), 6.04 (s, 1H), 4.23 (s, 1H), 4.08 (s, 2H), 3.80-3.76 (m, 6H), 3.70-3.68 (m, 1H), 3.54-3.52 (m, 1H), 3.10-3.05 (m, 7H), 2.91-2.88 (m, 2H), 2.81 (s, 1H), 2.29 (s, 3H), 2.17 (s, 2H), 1.99 (s, 2H), 1.70-1.65 (m, 4H), 1.62 (s, 2H), 1.57-1.53 (m, 3H), 1.39-1.29 (m, 9H), 1.27-1.23 (m, 2H), 1.13-1.08 (m, 7H), 0.98 (d, J=6.5 Hz, 3H), 0.84 (s, 1H).

### Example 139 Preparation of 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-4-{2-[(2R)-4-({5-methoxy-6- [(3R)-3-methylmorpholin-4-yl]pyridin-3-yl}methyl)-2-(2-methylphenyl)piperazin-1-yl]-7-azaspiro[3.5]nonan-7-yl}-N-[3-nitro-4-({[(1r,4r)-4-hydroxy-4-methylcyclohexyl]methyl}amino)benzenesulfonyl]benzamide

Synthesis of (3R)-4-(3-methoxy-5-{[(3R)-3-(2-methylphenyl)piperazin-1-yl]methyl}pyridin-2-yl)-3-methylmorpholine: Into an 8 mL vial were added (2R)-2-(2-methylphenyl)piperazine (70 mg, 0.4 mmol, 1.0 eq), 5-methoxy-6-[(3R)-3-methylmorpholin-4-yl]pyridine-3-carbaldehyde (93 mg, 0.4 mmol, 1.0 eq), NaBH₃CN (124 mg, 2.0 mmol, 5.0 eq), ZnCl₂ (270 mg, 2.0 mmol, 5.0 eq) and MeOH (2 mL). The resulting mixture was stirred for 4 hours at 25° C. The residue was purified by Prep-TLC (CH₂Cl₂/MeOH=10:1) to give (3R)-4-(3-methoxy-5-{[(3R)-3-(2-methylphenyl)piperazin-1-yl]methyl}pyridin-2-yl)-3-methylmorpholine as a colorless solid (40 mg, 25.4%). LC-MS (ESI, m/z) M+1: 397.

Synthesis of 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-4-{2-[(2R)-4-({5-methoxy-6-[(3R)-3-methylmorpholin-4-yl]pyridin-3-yl}methyl)-2-(2-methylphenyl)piperazin-1-yl]-7-azaspiro[3.5]nonan-7-yl}-N-[3-nitro-4-({[(1r,4r)-4-hydroxy-4-methylcyclohexyl]methyl}amino)benzenesulfonyl]benzamide: Into a mL 8 vial were added (3R)-4-(3-methoxy-5-{[(3R)-3-(2-methylphenyl)piperazin-1-yl]methyl}pyridin-2-yl)-3-methylmorpholine (40 mg, 0.1 mmol, 1.0 eq), 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-N-[3-nitro-4-({[(1r,4r)-4-hydroxy-4-methylcyclohexyl]methyl}amino)benzenesulfonyl]-4-{2-oxo-7-azaspiro[3.5]nonan-7-yl}benzamide (82 mg, 0.1 mmol, 1.0 eq), NaBH₃CN (32 mg, 0.5 mmol, 5.0 eq), ZnCl₂ (69 mg, 0.5 mmol, 5.0 eq) and MeOH (2 mL). The resulting mixture was stirred for 4 hours at 70° C. The reaction was quenched by the addition of water (2 mL), and extracted with CH₂Cl₂ (3×10 mL). The combined organic layers were washed with brine (10 mL), dried over anhydrous Na₂SO₄. After filtration, the filtrate was concentrated under vacuum. The residue was purified by Prep-TLC (CH₂Cl₂/MeOH=15:1) to give 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-4-{2-[(2R)-4-({5-methoxy-6-[(3R)-3-methylmorpholin-4-yl]pyridin-3-yl}methyl)-2-(2-methylphenyl)piperazin-1-yl]-7-azaspiro[3.5]nonan-7-yl}-N-[3-nitro-4-({[(1r,4r)-4-hydroxy-4-methylcyclohexyl]methyl}amino)benzenesulfonyl]benzamide assumed as a yellow solid (16 mg, 13.3%). LC-MS (ESI, m/z) M+1: 1195. ¹H NMR (300 MHz, Chloroform-d) δ 12.97-12.80 (bs, 1H), 12.58 (d, J=4.7 Hz, 1H), 8.65 (s, 1H), 8.46 (s, 1H), 8.38 (s, 1H), 8.33 (d, J=8.3 Hz, 1H), 8.11 (d, J=9.1 Hz, 1H), 7.81 (d, J=8.5 Hz, 1H), 7.66 (s, 1H), 7.18 (s, 2H), 7.09 (s, 1H), 6.95 (s, 1H), 6.84 (d, J=9.2 Hz, 1H), 6.71 (d, J=9.2 Hz, 1H), 6.62 (d, J=3.8 Hz, 1H), 6.43 (s, 1H), 6.11 (s, 1H), 4.64-4.41 (m, 3H), 3.94 (d, J=16.3 Hz, 4H), 3.86 (d, J=12.5 Hz, 3H), 3.78-3.61 (m, 3H), 3.53 (d, J=14.3 Hz, 6H), 3.38-3.17 (m, 5H), 3.06 (s, 6H), 2.87 (s, 2H), 2.73 (s, 3H), 2.41 (d, J=14.2 Hz, 5H), 2.13 (s, 3H), 1.98-1.68 (m, 8H), 1.49-1.33 (m, 5H), 1.33-1.04 (m, 5H).

### Example 140 Preparation of 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-4-[(1S,2R)-2-[(2R)-4-{[5-methoxy-6-(morpholin-4-yl)pyridin-3-yl]methyl}-2-(2-methylphenyl)piperazin-1-yl]-1-methyl-7-azaspiro[3.5]nonan-7-yl]-N-[3-nitro-4-({[(1r,4r)-4-hydroxy-4-methylcyclohexyl]methyl}amino)benzenesulfonyl]benzamide (assumed)

Synthesis of 4-[(1S,2S)-2-[(2R)-4-{[5-methoxy-6-(morpholin-4-yl)pyridin-3-yl]methyl}-2-(2-methylphenyl)piperazin-1-yl]-1-methyl-7-azaspiro[3.5]nonan-7-yl]-N-[3-nitro-4-({[(1r,4r)-4-hydroxy-4-methylcyclohexyl]methyl}amino)benzenesulfonyl]-2-[(3R,8S)-15-{[2-(trimethylsilyl)ethoxy]methyl}-2,5-dioxa-9,15,17-triazatetracyclo[8.7.0.0\hat{ }{3,8}.0\hat{ }{12,16}]heptadeca-1(10),11,13,16-tetraen-9-yl]benzamide (assumed) & 4-[(1S,2R)-2-[(2R)-4-{[5-methoxy-6-(morpholin-4-yl)pyridin-3-yl]methyl}-2-(2-methylphenyl)piperazin-1-yl]-1-methyl-7-azaspiro[3.5]nonan-7-yl]-N-[3-nitro-4-({[(1r,4r)-4-hydroxy-4-methylcyclohexyl]methyl}amino)benzenesulfonyl]-2-[(3R,8S)-15-{[2-(trimethylsilyl)ethoxy]methyl}-2,5-dioxa-9,15,17-triazatetracyclo[8.7.0.0\hat{ }{3,8}.0\hat{ }{12,16}]heptadeca-1(10),11,13,16-tetraen-9-yl]benzamide (assumed): Into an 8-mL sealed tube, were placed 4-{1-methyl-2-oxo-7-azaspiro[3.5]nonan-7-yl}-N-[3-nitro-4-({[(1r,4r)-4-hydroxy-4-methylcyclohexyl]methyl}amino)benzenesulfonyl]-2-[(3R,8S)-15-{[2-(trimethylsilyl)ethoxy]methyl}-2,5-dioxa-9,15,17-triazatetracyclo[8.7.0.0\hat{ }{3,8}.0\hat{ }{12,16}]heptadeca-1(10),11,13,16-tetraen-9-yl]benzamide (263 mg, 0.3 mmol, 1.0 eq), 4-(3-methoxy-5-{[(3R)-3-(2-methylphenyl)piperazin-1-yl]methyl}pyridin-2-yl)morpholine (105 mg, 0.3 mmol, 1.0 eq), NaBH₃CN (52 mg, 0.8 mmol, 3.0 eq), ZnCl₂ (112 mg, 0.8 mmol, 3.0 eq), MeOH (3 mL). The resulting solution was stirred for overnight at 70° C. The resulting mixture was then quenched by the addition of water (30 mL), and extracted with dichloromethane (2×30 mL). The combined organic layers was washed with brine (2×30 mL), dried over anhydrous Na₂SO₄. The resulting mixture was filtered, the filtrate was concentrated under vacuum. The residue was purified by reverse flash chromatography using the following conditions: column, C18 silica gel; mobile phase, CH₃CN in water (0.1% NH₃·H₂O), 10% to 50% gradient in 10 min; detector, UV 254 nm. Finally, 4-[(1S,2S)-2-[(2R)-4-{[5-methoxy-6-(morpholin-4-yl)pyridin-3-yl]methyl}-2-(2-methylphenyl)piperazin-1-yl]-1-methyl-7-azaspiro[3.5]nonan-7-yl]-N-[3-nitro-4-({[(1r,4r)-4-hydroxy-4-methylcyclohexyl]methyl}amino)benzenesulfonyl]-2-[(3R,8S)-15-{[2-(trimethylsilyl)ethoxy]methyl}-2,5-dioxa-9,15,17-triazatetracyclo[8.7.0.0\hat{ }{3,8}.0\hat{ }{12,16}]heptadeca-1(10),11,13,16-tetraen-9-yl]benzamide (assumed) was obtained as a yellow solid (60 mg, 16.5%) and 4-[(1S,2R)-2-[(2R)-4-{[5-methoxy-6-(morpholin-4-yl)pyridin-3-yl]methyl}-2-(2-methylphenyl)piperazin-1-yl]-1-methyl-7-azaspiro[3.5]nonan-7-yl]-N-[3-nitro-4-({[(1r,4r)-4-hydroxy-4-methylcyclohexyl]methyl}amino)benzenesulfonyl]-2-[(3R,8S)-15-{[2-(trimethylsilyl)ethoxy]methyl}-2,5-dioxa-9,15,17-triazatetracyclo[8.7.0.0\hat{ }{3,8}.0\hat{ }{12,16}]heptadeca-1(10),11,13,16-tetraen-9-yl]benzamide (assumed) was obtained as a yellow solid (60 mg, 16.5%). LC-MS (ESI, m/z) M+1: 1325.

Synthesis of 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-4-[(1S,2R)-2-[(2R)-4-{[5-methoxy-6-(morpholin-4-yl)pyridin-3-yl]methyl}-2-(2-methylphenyl)piperazin-1-yl]-1-methyl-7-azaspiro[3.5]nonan-7-yl]-N-[3-nitro-4-({[(1r,4r)-4-hydroxy-4-methylcyclohexyl]methyl}amino)benzenesulfonyl]benzamide (assumed): Into an 8 mL vial, were placed 4-[(1S,2R)-2-[(2R)-4-{[5-methoxy-6-(morpholin-4-yl)pyridin-3-yl]methyl}-2-(2-methylphenyl)piperazin-1-yl]-1-methyl-7-azaspiro[3.5]nonan-7-yl]-N-[3-nitro-4-({[(1r,4r)-4-hydroxy-4-methylcyclohexyl]methyl}amino)benzenesulfonyl]-2-[(3R,8S)-15-{[2-(trimethylsilyl)ethoxy]methyl}-2,5-dioxa-9,15,17-triazatetracyclo[8.7.0.0\hat{ }{3,8}.0\hat{ }{12,16}]heptadeca-1(10),11,13,16-tetraen-9-yl]benzamide (assumed) (55 mg, 0.04 mmol, 1.0 eq), ethylenediamine (50 mg, 0.8 mmol, 20.0 eq), TBAF in THE (1 M, 0.5 mL). The resulting solution was stirred for 5 hours at 80° C. The resulting mixture was then quenched by the addition of water (30 mL), and extracted with ethyl acetate (2×30 mL). The combined organic layers was washed with brine (2×30 mL), dried over anhydrous Na₂SO₄. After filtration, the filtrate was concentrated under vacuum. The crude residue was applied onto a silica gel column and eluted with dichloromethane/methanol=15:1 to give 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-4-[(1S,2R)-2-[(2R)-4-{[5-methoxy-6-(morpholin-4-yl)pyridin-3-yl]methyl}-2-(2-methylphenyl)piperazin-1-yl]-1-methyl-7-azaspiro[3.5]nonan-7-yl]-N-[3-nitro-4-({[(1r,4r)-4-hydroxy-4-methylcyclohexyl]methyl}amino)benzenesulfonyl]benzamide (assumed) as a yellow solid. (2.6 mg, 5.2%). LC-MS (ESI, m/z) M+1: 1195. ¹H NMR (400 MHz, Methanol-d₄) δ 8.52-8.46 (m, 1H), 7.92-7.85 (m, 1H), 7.72-7.67 (m, 1H), 7.53-7.40 (m, 2H), 7.31-7.27 (m, 1H), 7.20-7.11 (m, 3H), 7.09-7.05 (m, 1H), 6.98-6.85 (m, 1H), 6.72-6.62 (m, 2H), 6.48 (s, 1H), 6.05-5.94 (m, 1H), 4.35-4.23 (m, 2H), 3.97-3.90 (m, 1H), 3.87 (s, 3H), 3.85-3.78 (m, 4H), 3.77-3.64 (m, 2H), 3.64-3.44 (m, 6H), 3.28-3.21 (m, 2H), 3.18-3.09 (m, 1H), 3.00-2.93 (m, 1H), 2.74-2.54 (m, 4H), 2.42-2.32 (m, 4H), 2.29-2.17 (m, 1H), 2.09-1.94 (m, 1H), 1.89-1.80 (m, 2H), 1.79-1.64 (m, 4H), 1.63-1.47 (m, 5H), 1.37-1.21 (m, 12H), 1.02-0.84 (m, 5H).

### Example 141 Preparation of 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-4-[(1S,2S)-2-[(2R)-4-{[5-methoxy-6-(morpholin-4-yl)pyridin-3-yl]methyl}-2-(2-methylphenyl)piperazin-1-yl]-1-methyl-7-azaspiro[3.5]nonan-7-yl]-N-[3-nitro-4-({[(1r,4r)-4-hydroxy-4-methylcyclohexyl]methyl}amino)benzenesulfonyl]benzamide (assumed)

Synthesis of 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-4-[(1S,2S)-2-[(2R)-4-{[5-methoxy-6-(morpholin-4-yl)pyridin-3-yl]methyl}-2-(2-methylphenyl)piperazin-1-yl]-1-methyl-7-azaspiro[3.5]nonan-7-yl]-N-[3-nitro-4-({[(1r,4r)-4-hydroxy-4-methylcyclohexyl]methyl}amino)benzenesulfonyl]benzamide (assumed): Into an 8 mL vial, were placed 4-[(1S,2S)-2-[(2R)-4-{[5-methoxy-6-(morpholin-4-yl)pyridin-3-yl]methyl}-2-(2-methylphenyl)piperazin-1-yl]-1-methyl-7-azaspiro[3.5]nonan-7-yl]-N-[3-nitro-4-({[(1r,4r)-4-hydroxy-4-methylcyclohexyl]methyl}amino)benzenesulfonyl]-2-[(3R,8S)-15-{[2-(trimethylsilyl)ethoxy]methyl}-2,5-dioxa-9,15,17-triazatetracyclo[8.7.0.0\hat{ }{3,8}.0\hat{ }{12,16}]heptadeca-1(10),11,13,16-tetraen-9-yl]benzamide (assumed) (55 mg, 0.04 mmol, 1.0 eq), ethylenediamine (50 mg, 0.8 mmol, 20.0 eq), TBAF in THE (1 M, 0.5 mL). The resulting solution was stirred for 5 hours at 80° C. The resulting mixture was then quenched by the addition of water (30 mL), and extracted with ethyl acetate (2×30 mL). The combined organic layers was washed with brine (2×30 mL), dried over anhydrous Na₂SO₄. After filtration, the filtrate was concentrated under vacuum. The crude residue was applied onto a silica gel column and eluted with dichloromethane/methanol=15:1 to give 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-4-[(1S,2S)-2-[(2R)-4-{[5-methoxy-6-(morpholin-4-yl)pyridin-3-yl]methyl}-2-(2-methylphenyl)piperazin-1-yl]-1-methyl-7-azaspiro[3.5]nonan-7-yl]-N-[3-nitro-4-({[(1r,4r)-4-hydroxy-4-methylcyclohexyl]methyl}amino)benzenesulfonyl]benzamide (assumed) as a yellow solid. (2.9 mg, 5.9%). LC-MS (ESI, m/z) M+1: 1195. ¹H NMR (400 MHz, Methanol-d₄) δ 8.52-8.46 (m, 1H), 7.92-7.85 (m, 1H), 7.71-7.65 (m, 1H), 7.55-7.43 (m, 2H), 7.32-7.26 (m, 1H), 7.20-7.10 (m, 3H), 7.09-7.06 (m, 1H), 7.00-6.88 (m, 1H), 6.74-6.65 (m, 2H), 6.49 (s, 1H), 6.05-5.94 (m, 1H), 4.35-4.23 (m, 2H), 3.97-3.90 (m, 1H), 3.87 (s, 3H), 3.85-3.78 (m, 4H), 3.77-3.64 (m, 2H), 3.64-3.44 (m, 6H), 3.28-3.21 (m, 2H), 3.18-3.09 (m, 1H), 3.00-2.93 (m, 1H), 2.74-2.54 (m, 4H), 2.42-2.32 (m, 4H), 2.29-2.17 (m, 1H), 2.09-1.94 (m, 1H), 1.89-1.80 (m, 2H), 1.79-1.64 (m, 4H), 1.63-1.47 (m, 5H), 1.37-1.21 (m, 12H), 1.02-0.84 (m, 5H).

### Example 142 Preparation of 4-(2-((R)-4-((5-methoxy-6-morpholinopyridin-3-yl)methyl)-2-(o-tolyl)piperazin-1-yl)-7-azaspiro[3.5]nonan-7-yl)-N—(((S)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide (assumed)

Synthesis of 4-(2-((R)-4-((5-methoxy-6-morpholinopyridin-3-yl)methyl)-2-(o-tolyl)piperazin-1-yl)-7-azaspiro[3.5]nonan-7-yl)-N—(((S)-5-nitro-3-(tetrahydro-2H-pyran-4-yl)-3,4-dihydro-2H-benzo[b][1,4]oxazin-7-yl)sulfonyl)-2-((5aS,9aR)-1-((2-(trimethylsilyl)ethoxy)methyl)-6,7,9,9a-tetrahydro-1H-pyrano[3,4-b]pyrrolo[3′,2′:5,6]pyrido[3,2-e][1,4]oxazin-5(5aH)-yl)benzamide (assumed): Into an 8 mL vial were added 4-(2-((R)-4-((5-methoxy-6-morpholinopyridin-3-yl)methyl)-2-(o-tolyl)piperazin-1-yl)-7-azaspiro[3.5]nonan-7-yl)-2-((5aS,9aR)-1-((2-(trimethylsilyl)ethoxy)methyl)-6,7,9,9a-tetrahydro-1H-pyrano[3,4-b]pyrrolo[3′,2′:5,6]pyrido[3,2-e][1,4]oxazin-5(5aH)-yl)benzoic acid (33 mg, 0.1 mmol, 1.0 eq), (S)-5-nitro-3-(tetrahydro-2H-pyran-4-yl)-3,4-dihydro-2H-benzo[b][1,4]oxazine-7-sulfonamide (assumed) (33 mg, 0.1 mmol, 1.0 eq), EDCl (37 mg, 0.2 mmol, 2.0 eq), DMAP (47 mg, 0.4 mmol, 4.0 eq) and CH₂Cl₂ (2 mL). The resulting mixture was stirred for 2 hours at 25° C. The resulting mixture was diluted with water (5 mL), and extracted with CH₂Cl₂ (3×10 mL). The combined organic layers were washed with brine (3×10 mL), dried over anhydrous Na₂SO₄. After filtration, the filtrate was concentrated under vacuum. The residue was purified by Prep-TLC (CH₂Cl₂/MeOH=20:1) to give 4-(2-((R)-4-((5-methoxy-6-morpholinopyridin-3-yl)methyl)-2-(o-tolyl)piperazin-1-yl)-7-azaspiro[3.5]nonan-7-yl)-N—(((S)-5-nitro-3-(tetrahydro-2H-pyran-4-yl)-3,4-dihydro-2H-benzo[b][1,4]oxazin-7-yl)sulfonyl)-2-((5aS,9aR)-1-((2-(trimethylsilyl)ethoxy)methyl)-6,7,9,9a-tetrahydro-1H-pyrano[3,4-b]pyrrolo[3′,2′:5,6]pyrido[3,2-e][1,4]oxazin-5(5aH)-yl)benzamide (assumed) as a yellow solid (60 mg, 45.6%). LC-MS (ESI, m/z) M+1: 1311.

Synthesis of 4-(2-((R)-4-((5-methoxy-6-morpholinopyridin-3-yl)methyl)-2-(o-tolyl)piperazin-1-yl)-7-azaspiro[3.5]nonan-7-yl)-N—(((S)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide (assumed): Into an 8 mL vial were added 4-(2-((R)-4-((5-methoxy-6-morpholinopyridin-3-yl)methyl)-2-(o-tolyl)piperazin-1-yl)-7-azaspiro[3.5]nonan-7-yl)-N—(((S)-5-nitro-3-(tetrahydro-2H-pyran-4-yl)-3,4-dihydro-2H-benzo[b][1,4]oxazin-7-yl)sulfonyl)-2-((5aS,9aR)-1-((2-(trimethylsilyl)ethoxy)methyl)-6,7,9,9a-tetrahydro-1H-pyrano[3,4-b]pyrrolo[3′,2′:5,6]pyrido[3,2-e][1,4]oxazin-5(5aH)-yl)benzamide (assumed) (50 mg, 0.04 mmol, 1.0 eq), ethylenediamine (89 mg, 1.5 mmol, 40.0 eq) and TBAF in THE (5 M, 2 mL). The resulting mixture was stirred for 4 hours at 70° C. The reaction was quenched by the addition of water (2 mL), and extracted with CH₂Cl₂ (3×5 mL). The combined organic layers were washed with brine (3 mL), dried over anhydrous Na₂SO₄. After filtration, the filtrate was concentrated under vacuum. The residue was purified by Prep-TLC (CH₂Cl₂/MeOH=17:1) to give 4-(2-((R)-4-((5-methoxy-6-morpholinopyridin-3-yl)methyl)-2-(o-tolyl)piperazin-1-yl)-7-azaspiro[3.5]nonan-7-yl)-N—(((S)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide (assumed) as a yellow solid (8 mg, 15.5%). LC-MS (ESI, m/z) M+1: 1181. ¹H NMR (300 MHz, Chloroform-d) δ 12.60 (s, 1H), 8.55 (s, 1H), 8.32 (d, J=8.2 Hz, 1H), 8.03 (d, J=9.0 Hz, 1H), 7.80 (s, 1H), 7.46 (s, 1H), 7.07 (d, J=15.5 Hz, 3H), 6.98 (s, 1H), 6.75 (d, J=7.8 Hz, 3H), 6.68 (d, J=5.8 Hz, 1H), 6.43 (s, 1H), 6.13 (s, 1H), 4.64 (d, J=18.4 Hz, 3H), 4.19-3.91 (m, 7H), 3.85 (s, 3H), 3.78 (s, 3H), 3.55-3.29 (m, 11H), 3.15 (s, 3H), 2.11-2.06 (m, 8H), 1.45 (s, 3H), 1.41-1.32 (m, 6H), 1.29 (d, J=8.0 Hz, 6H), 1.04-0.78 (m, 5H).

### Example 143 Preparation of 4-(2-((R)-4-((5-methoxy-6-morpholinopyridin-3-yl)methyl)-2-(o-tolyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide (assumed)

Synthesis of 4-(2-((R)-4-((5-methoxy-6-morpholinopyridin-3-yl)methyl)-2-(o-tolyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-1-((2-(trimethylsilyl)ethoxy)methyl)-6,7,9,9a-tetrahydro-1H-pyrano[3,4-b]pyrrolo[3′,2′:5,6]pyrido[3,2-e][1,4]oxazin-5(5aH)-yl)benzamide (assumed): Into an 8 mL vial were added 4-(2-((R)-4-((5-methoxy-6-morpholinopyridin-3-yl)methyl)-2-(o-tolyl)piperazin-1-yl)-7-azaspiro[3.5]nonan-7-yl)-2-((5aS,9aR)-1-((2-(trimethylsilyl)ethoxy)methyl)-6,7,9,9a-tetrahydro-1H-pyrano[3,4-b]pyrrolo[3′,2′:5,6]pyrido[3,2-e][1,4]oxazin-5(5aH)-yl)benzoic acid (100 mg, 0.1 mmol, 1.0 eq), (R)-5-nitro-3-(tetrahydro-2H-pyran-4-yl)-3,4-dihydro-2H-benzo[b][1,4]oxazine-7-sulfonamide (assumed) (33 mg, 0.1 mmol, 1.0 eq), EDCl (74 mg, 0.4 mmol, 4.0 eq), DMAP (24 mg, 0.1 mmol, 2.0 eq) and CH₂Cl₂ (2 mL). The resulting mixture was stirred for 2 hours at 25° C. The resulting mixture was diluted with water (5 mL), and extracted with CH₂Cl₂ (3×10 mL). The combined organic layers were washed with brine (3×10 mL), dried over anhydrous Na₂SO₄. After filtration, the filtrate was concentrated under vacuum. The residue was purified by Prep-TLC (CH₂Cl₂/MeOH=20:1) to give 4-(2-((R)-4-((5-methoxy-6-morpholinopyridin-3-yl)methyl)-2-(o-tolyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-1-((2-(trimethylsilyl)ethoxy)methyl)-6,7,9,9a-tetrahydro-1H-pyrano[3,4-b]pyrrolo[3′,2′:5,6]pyrido[3,2-e][1,4]oxazin-5(5aH)-yl)benzamide (assumed) as a yellow solid (68 mg, 51.7%). LC-MS (ESI, m/z) M+1: 1311.

Synthesis of 4-(2-((R)-4-((5-methoxy-6-morpholinopyridin-3-yl)methyl)-2-(o-tolyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide (assumed): Into an 8 mL vial were added 4-(2-((R)-4-((5-methoxy-6-morpholinopyridin-3-yl)methyl)-2-(o-tolyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-1-((2-(trimethylsilyl)ethoxy)methyl)-6,7,9,9a-tetrahydro-1H-pyrano[3,4-b]pyrrolo[3′,2′:5,6]pyrido[3,2-e][1,4]oxazin-5(5aH)-yl)benzamide (assumed) (50 mg, 0.04 mmol, 1.0 eq), ethylenediamine (88 mg, 1.5 mmol, 40.0 eq), and TBAF in THE (3 M, 2 mL). The resulting mixture was stirred for 4 hours at 70° C. The reaction was quenched by the addition of water (2 mL), and extracted with CH₂Cl₂ (3×5 mL). The combined organic layers were washed with brine (3 mL), dried over anhydrous Na₂SO₄. After filtration, the filtrate was concentrated under vacuum. The residue was purified by Prep-TLC (CH₂Cl₂/MeOH=17:1) to give 4-(2-((R)-4-((5-methoxy-6-morpholinopyridin-3-yl)methyl)-2-(o-tolyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide (assumed) as a yellow solid (8 mg, 17.7%). LC-MS (ESI, m/z) M+1: 1181. ¹H NMR (300 MHz, Chloroform-d) δ 12.62 (d, J=4.4 Hz, 1H), 8.43 (d, J=11.1 Hz, 3H), 8.29 (s, 1H), 8.13 (d, J=9.0 Hz, 1H), 7.80 (s, 1H), 7.71 (d, J=18.0 Hz, 1H), 7.46 (d, J=2.2 Hz, 1H), 7.06 (d, J=14.5 Hz, 1H), 7.00 (d, J=9.0 Hz, 1H), 6.85 (d, J=8.7 Hz, 2H), 6.67 (d, J=6.3 Hz, 1H), 6.43 (s, 1H), 6.13 (s, 1H), 4.79-4.42 (m, 4H), 4.33-3.93 (m, 4H), 3.85 (s, 2H), 3.64-3.31 (m, 9H), 3.08 (d, J=17.7 Hz, 3H), 2.82-2.48 (m, 3H), 2.48-1.93 (m, 9H), 1.63 (d, J=95.3 Hz, 9H), 1.45-1.11 (m, 8H), 1.17-0.76 (m, 6H).

### Example 144 Preparation of 4-(2-((S)-2-(2-isopropoxyphenyl)-4-((5-methoxy-6-morpholinopyridin-3-yl)methyl)piperazin-1-yl)-7-azaspiro[3.5]nonan-7-yl)-N-((3-nitro-4-(((tetrahydro-2H-pyran-4-yl)methyl)amino)phenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide

Synthesis of 4-(2-((R)-4-((5-methoxy-6-morpholinopyridin-3-yl)methyl)-2-(o-tolyl)piperazin-1-yl)-7-azaspiro[3.5]nonan-7-yl)-N-((3-nitro-4-(((tetrahydro-2H-pyran-4-yl)methyl)amino)phenyl)sulfonyl)-2-((5aS,9aR)-1-((2-(trimethylsilyl)ethoxy)methyl)-6,7,9,9a-tetrahydro-1H-pyrano[3,4-b]pyrrolo[3′,2′:5,6]pyrido[3,2-e][1,4]oxazin-5(5aH)-yl)benzamide: Into an 8 mL vial were added 4-(2-((R)-4-((5-methoxy-6-morpholinopyridin-3-yl)methyl)-2-(o-tolyl)piperazin-1-yl)-7-azaspiro[3.5]nonan-7-yl)-2-((5aS,9aR)-1-((2-(trimethylsilyl)ethoxy)methyl)-6,7,9,9a-tetrahydro-1H-pyrano[3,4-b]pyrrolo[3′,2′:5,6]pyrido[3,2-e][1,4]oxazin-5(5aH)-yl)benzoic acid (90 mg, 0.09 mmol, 1.0 eq), 3-nitro-4-(((tetrahydro-2H-pyran-4-yl)methyl)amino)benzenesulfonamide (28 mg, 0.09 mmol, 1.0 eq), DMAP (43 mg, 0.3 mmol, 4.0 eq) and CH₂Cl₂ (2 mL). The resulting mixture was stirred for 2 hours at 25° C. The resulting mixture was diluted with water (5 mL), and extracted with CH₂Cl₂ (3×10 mL). The combined organic layers were washed with brine (3×10 mL), dried over anhydrous Na₂SO₄. After filtration, the filtrate was concentrated under vacuum. The residue was purified by Prep-TLC (CH₂Cl₂/MeOH=20:1) to give 4-(2-((R)-4-((5-methoxy-6-morpholinopyridin-3-yl)methyl)-2-(o-tolyl)piperazin-1-yl)-7-azaspiro[3.5]nonan-7-yl)-N-((3-nitro-4-(((tetrahydro-2H-pyran-4-yl)methyl)amino)phenyl)sulfonyl)-2-((5aS,9aR)-1-((2-(trimethylsilyl)ethoxy)methyl)-6,7,9,9a-tetrahydro-1H-pyrano[3,4-b]pyrrolo[3′,2′:5,6]pyrido[3,2-e][1,4]oxazin-5(5aH)-yl)benzamide as a yellow solid (70 mg, 57.9%). LC-MS (ESI, m/z) M+1: 1283.

Synthesis of 4-(2-((S)-2-(2-isopropoxyphenyl)-4-((5-methoxy-6-morpholinopyridin-3-yl)methyl)piperazin-1-yl)-7-azaspiro[3.5]nonan-7-yl)-N-((3-nitro-4-(((tetrahydro-2H-pyran-4-yl)methyl)amino)phenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide: Into an 8 mL vial were added 4-(2-((S)-2-(2-isopropoxyphenyl)-4-((5-methoxy-6-morpholinopyridin-3-yl)methyl)piperazin-1-yl)-7-azaspiro[3.5]nonan-7-yl)-N-((3-nitro-4-(((tetrahydro-2H-pyran-4-yl)methyl)amino)phenyl)sulfonyl)-2-((5aS,9aR)-1-((2-(trimethylsilyl)ethoxy)methyl)-6,7,9,9a-tetrahydro-1H-pyrano[3,4-b]pyrrolo[3′,2′:5,6]pyrido[3,2-e][1,4]oxazin-5(5aH)-yl)benzamide (65 mg, 0.05 mmol, 1.0 eq), ethylenediamine (56 mg, 0.9 mmol, 20.0 eq), TBAF in THE (246 mg, 0.9 mmol, 20.0 eq). The resulting mixture was stirred for 4 hours at 70° C. The reaction was quenched by the addition of water (2 mL) and extracted with CH₂Cl₂ (3×10 mL). The combined organic layers were washed with brine (3×10 mL), dried over anhydrous Na₂SO₄. After filtration, the filtrate was concentrated under vacuum. The residue was purified by Prep-TLC (CH₂Cl₂/MeOH=10:1) to give 4-(2-((S)-2-(2-isopropoxyphenyl)-4-((5-methoxy-6-morpholinopyridin-3-yl)methyl)piperazin-1-yl)-7-azaspiro[3.5]nonan-7-yl)-N-((3-nitro-4-(((tetrahydro-2H-pyran-4-yl)methyl)amino)phenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide as a yellow solid (7 mg, 11.9%). LC-MS (ESI, m/z) M+1: 1153. ¹H NMR (400 MHz, Chloroform-d) δ 12.60 (s, 1H), 12.50 (d, J=6.2 Hz, 1H), 8.35 (s, 1H), 8.24 (s, 1H), 8.20 (d, J=2.2 Hz, 1H), 8.13 (d, J=9.2 Hz, 1H), 7.91 (s, 1H), 7.54 (s, 1H), 7.09 (s, 1H), 7.05 (d, J=7.5 Hz, 1H), 6.99 (s, 1H), 6.84 (t, J=8.7 Hz, 3H), 6.68 (d, J=8.4 Hz, 1H), 6.43 (s, 1H), 6.14 (s, 1H), 4.75-4.57 (m, 2H), 4.57-4.38 (m, 1H), 4.18 (dd, J=11.3, 3.2 Hz, 1H), 4.11-3.92 (m, 3H), 3.85 (d, J=4.8 Hz, 3H), 3.58 (s, 3H), 3.54-3.25 (m, 6H), 3.18 (d, J=23.7 Hz, 3H), 2.70 (dd, J=60.7, 11.0 Hz, 3H), 2.29-1.98 (m, 6H), 1.98-1.76 (m, 5H), 1.50-1.40 (m, 6H), 1.37 (d, J=6.0 Hz, 3H), 1.30 (d, J=11.1 Hz, 9H).

### Example 145 Preparation of 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-4-{2-[(2R)-4-{[5-isopropoxy-6-(morpholin-4-yl)pyridin-3-yl]methyl}-2-(2-methylphenyl)piperazin-1-yl]-7-azaspiro[3.5]nonan-7-yl}-N-[3-nitro-4-({[(1r,4r)-4-hydroxy-4-methylcyclohexyl]methyl}amino)benzenesulfonyl]benzamide

Synthesis of 4-(3-isopropoxy-5-{[(3R)-3-(2-methylphenyl)piperazin-1-yl]methyl}pyridin-2-yl)morpholine: Into an 8-mL sealed tube purged and maintained with an inert atmosphere of nitrogen, were placed 5-isopropoxy-6-(morpholin-4-yl)pyridine-3-carbaldehyde (140 mg, 0.6 mmol, 1.0 eq), (2R)-2-(2-methylphenyl)piperazine (99 mg, 0.6 mmol, 1.0 eq), NaBH(OAc)₃ (356 mg, 1.7 mmol, 3.0 eq), DCE (1.5 mL). The resulting solution was stirred for 2 hours at 25° C. The resulting mixture was then quenched by the addition of water (30 mL), and extracted with dichloromethane (2×30 mL). The combined organic layers was washed with brine (2×30 mL), dried over anhydrous Na₂SO₄. After filtration, the filtrate was concentrated under vacuum. The crude residue was applied onto a silica gel column and eluted with dichloromethane/methanol=10:1 to give 4-(3-isopropoxy-5-{[(3R)-3-(2-methylphenyl)piperazin-1-yl]methyl}pyridin-2-yl)morpholine as a light yellow solid (130 mg, 56.6%). LC-MS (ESI, m/z) M+1: 411.

Synthesis of 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-4-{2-[(2R)-4-{[5-isopropoxy-6-(morpholin-4-yl)pyridin-3-yl]methyl}-2-(2-methylphenyl)piperazin-1-yl]-7-azaspiro[3.5]nonan-7-yl}-N-[3-nitro-4-({[(1r,4r)-4-hydroxy-4-methylcyclohexyl]methyl}amino)benzenesulfonyl]benzamide: Into an 8-mL sealed tube, were placed 4-(3-isopropoxy-5-{[(3R)-3-(2-methylphenyl)piperazin-1-yl]methyl}pyridin-2-yl)morpholine (20 mg, 0.05 mmol, 1.0 eq), 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-N-[3-nitro-4-({[(1r,4r)-4-hydroxy-4-methylcyclohexyl]methyl}amino)benzenesulfonyl]-4-{2-oxo-7-azaspiro[3.5]nonan-7-yl}benzamide (40 mg, 0.05 mmol, 1.0 eq), ZnCl₂ (20 mg, 0.1 mmol, 3.0 eq), NaBH₃CN (9 mg, 0.1 mmol, 3.0 eq), MeOH (0.5 mL). The resulting solution was stirred for 4 hours at 70° C. The resulting mixture was then quenched by the addition of water (30 mL), and extracted with dichloromethane (2×30 mL). The combined organic layers was washed with brine (2×30 mL), dried over anhydrous Na₂SO₄. After filtration, the filtrate was concentrated under vacuum. The crude residue was applied onto a silica gel column and eluted with dichloromethane/methanol=15:1 to give 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-4-{2-[(2R)-4-{[5-isopropoxy-6-(morpholin-4-yl)pyridin-3-yl]methyl}-2-(2-methylphenyl)piperazin-1-yl]-7-azaspiro[3.5]nonan-7-yl}-N-[3-nitro-4-({[(1r,4r)-4-hydroxy-4-methylcyclohexyl]methyl}amino)benzenesulfonyl]benzamide as a yellow solid (10 mg, 16.8%). LC-MS (ESI, m/z) M+1: 1209. ¹H NMR (400 MHz, Methanol-d₄) δ 8.53-8.46 (m, 1H), 7.89-7.66 (m, 2H), 7.51-7.39 (m, 2H), 7.32-7.21 (m, 2H), 7.19-7.10 (m, 3H), 7.09-7.04 (s, 1H), 6.98-6.89 (m, 1H), 6.71-6.63 (m, 1H), 6.55-6.46 (m, 1H), 6.05-5.95 (m, 1H), 4.72-4.57 (m, 2H), 4.37-4.19 (m, 2H), 3.97-3.87 (m, 1H), 3.85-3.77 (m, 6H), 3.73-3.57 (m, 2H), 3.55-3.43 (m, 4H), 3.29-3.22 (m, 3H), 3.18-3.05 (m, 3H), 3.02-2.91 (m, 2H), 2.70-2.60 (m, 1H), 2.41-2.31 (m, 6H), 2.27-2.14 (m, 2H), 1.93-1.80 (m, 3H), 1.79-1.67 (m, 4H), 1.61-1.40 (m, 9H), 1.38-1.29 (m, 6H), 1.28-1.23 (m, 3H), 1.20-1.11 (m, 1H), 0.96-0.83 (m, 2H).

### Example 146 Preparation of 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-4-{2-[(2R)-4-{[2-methyl-7-(morpholin-4-yl)furo[2,3-c]pyridin-4-yl]methyl}-2-(2-methylphenyl)piperazin-1-yl]-7-azaspiro[3.5]nonan-7-yl}-N-[3-nitro-4-({[(1r,4r)-4-hydroxy-4-methylcyclohexyl]methyl}amino)benzenesulfonyl]benzamide

Synthesis of 4-(2-methyl-4-{[(3R)-3-(2-methylphenyl)piperazin-1-yl]methyl}furo[2,3-c]pyridin-7-yl)morpholine: Into a 20-mL sealed-tube, were placed (2R)-2-(2-methylphenyl)piperazine (100 mg, 0.6 mmol, 1.0 eq), 2-methyl-7-(morpholin-4-yl)furo[2,3-c]pyridine-4-carbaldehyde (126 mg, 0.5 mmol, 0.9 eq), DCE (3 mL), NaBH(OAc)₃ (361 mg, 1.7 mmol, 3.0 eq). The resulting solution was stirred for 6 hours at 25° C. The resulting mixture was then quenched by the addition of water (20 mL), and extracted with dichloromethane/methanol (20:1, 2×20 mL). The combined organic layers was washed with brine (2×40 mL), dried over anhydrous Na₂SO₄. After filtration, the filtrate was concentrated under vacuum. The crude residue was purified onto a silica gel column and eluted with dichloromethane/methanol=10:1 to give 4-(2-methyl-4-{[(3R)-3-(2-methylphenyl)piperazin-1-yl]methyl}furo[2,3-c]pyridin-7-yl)morpholine as an off-white solid (120 mg, 52.0%). LC-MS (ESI, m/z) M+1: 407.

Synthesis of 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-4-{2-[(2R)-4-{[2-methyl-7-(morpholin-4-yl)furo[2,3-c]pyridin-4-yl]methyl}-2-(2-methylphenyl)piperazin-1-yl]-7-azaspiro[3.5]nonan-7-yl}-N-[3-nitro-4-({[(1r,4r)-4-hydroxy-4-methylcyclohexyl]methyl}amino)benzenesulfonyl]benzamide: Into an 8-mL sealed-tube, were placed 4-(2-methyl-4-{[(3R)-3-[(2Z)-penta-2,4-dien-1-yl]piperazin-1-yl]methyl}furo[2,3-c]pyridin-7-yl)morpholine (19 mg, 0.05 mmol, 1.0 eq), 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-N-[3-nitro-4-({[(1r,4r)-4-hydroxy-4-methylcyclohexyl]methyl}amino)benzenesulfonyl]-4-{2-oxo-7-azaspiro[3.5]nonan-7-yl}benzamide (40 mg, 0.05 mmol, 1.0 eq), MeOH (1 mL), ZnCl₂ (20 mg, 0.1 mmol, 3.0 eq), NaBH₃CN (9 mg, 0.1 mmol, 3.0 eq). The resulting solution was stirred for 6 hours at 70° C. The resulting mixture was then quenched by the addition of water (20 mL), and extracted with dichloromethane/methanol (10:1, 2×30 mL). The combined organic layers was washed with brine (2×20 mL), dried over anhydrous Na₂SO₄. After filtration, the filtrate was concentrated under vacuum. The residue was purified by Prep-TLC (dichloromethane/methanol=15:1) to give 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-4-{2-[(2R)-4-{[2-methyl-7-(morpholin-4-yl)furo[2,3-c]pyridin-4-yl]methyl}-2-(2-methylphenyl)piperazin-1-yl]-7-azaspiro[3.5]nonan-7-yl}-N-[3-nitro-4-({[(1r,4r)-4-hydroxy-4-methylcyclohexyl]methyl}amino)benzenesulfonyl]benzamide as a light yellow solid (12 mg, 20.3%). LC-MS (ESI, m/z) M+1: 1205. ¹H NMR (400 MHz, Methanol-d₄) δ 8.49 (dd, J=10.0, 2.2 Hz, 1H), 7.88 (d, J=9.0 Hz, 1H), 7.74 (s, 1H), 7.48 (dd, J=9.2, 2.4 Hz, 1H), 7.43 (br, 1H), 7.16-7.11 (m, 3H), 7.07 (d, J=3.4 Hz, 1H), 6.97-6.87 (m, 1H), 6.73-6.65 (m, 3H), 6.48 (s, 1H), 6.01 (d, J=3.4 Hz, 1H), 4.29 (d, J=6.8 Hz, 2H), 3.92 (d, J=8.8 Hz, 1H), 3.85 (t, J=4.8 Hz, 4H), 3.73 (q, J=4.4 Hz, 8H), 3.55-3.43 (m, 2H), 3.27-3.24 (m, 2H), 3.17-2.97 (m, 8H), 2.71 (d, J=12.0 Hz, 1H), 2.50 (d, J=1.0 Hz, 3H), 2.47-2.31 (m, 6H), 1.84 (d, J=14.2 Hz, 3H), 1.73 (d, J=11.8 Hz, 4H), 1.56-1.37 (m, 6H), 1.33-1.29 (m, 4H), 1.27-1.24 (m, 4H).

### Example 147 Preparation of 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-4-{2-[(2S)-2-(2-isopropoxyphenyl)-4-{[2-methyl-7-(morpholin-4-yl)furo[2,3-c]pyridin-4-yl]methyl}piperazin-1-yl]-7-azaspiro[3.5]nonan-7-yl}-N-[3-nitro-4-({[(1r,4r)-4-hydroxy-4-methylcyclohexyl]methyl}amino)benzenesulfonyl]benzamide (assumed)

Synthesis of 4-(4-{[(3S)-3-(2-isopropoxyphenyl)piperazin-1-yl]methyl}-2-methylfuro[2,3-c]pyridin-7-yl)morpholine (assumed): Into a 20-mL sealed-tube, were placed 2-methyl-7-(morpholin-4-yl)furo[2,3-c]pyridine-4-carbaldehyde (101 mg, 0.4 mmol, 0.9 eq), (2S)-2-(2-isopropoxyphenyl)piperazine (assumed) (100 mg, 0.5 mmol, 1.0 eq), DCE (2 mL), NaBH(OAc)₃ (289 mg, 1.4 mmol, 3.0 eq). The resulting solution was stirred for 4 hours at 25° C. The resulting mixture was then quenched by the addition of water (20 mL), extracted with dichloromethane/methanol (20:1, 2×20 mL). The combined organic layers was washed with brine (2×40 mL), dried over anhydrous Na₂SO₄. After filtration, the filtrate was concentrated under vacuum. The crude residue was purified onto a silica gel column and eluted with dichloromethane/methanol=10:1 to give 4-(4-{[(3S)-3-(2-isopropoxyphenyl)piperazin-1-yl]methyl}-2-methylfuro[2,3-c]pyridin-7-yl)morpholine (assumed) as an off-white solid (130 mg, 63.6%). LC-MS (ESI, m/z) M+1: 451.

Synthesis of 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-4-{2-[(2S)-2-(2-isopropoxyphenyl)-4-{[2-methyl-7-(morpholin-4-yl)furo[2,3-c]pyridin-4-yl]methyl}piperazin-1-yl]-7-azaspiro[3.5]nonan-7-yl}-N-[3-nitro-4-({[(1r,4r)-4-hydroxy-4-methylcyclohexyl]methyl}amino)benzenesulfonyl]benzamide (assumed): Into an 8-mL sealed-tube, were placed 4-(4-{[(3S)-3-(2-isopropoxyphenyl)piperazin-1-yl]methyl}-2-methylfuro[2,3-c]pyridin-7-yl)morpholine (assumed) (22 mg, 0.05 mmol, 1.0 eq), 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-N-[3-nitro-4-({[(1r,4r)-4-hydroxy- 4-methylcyclohexyl]methyl}amino)benzenesulfonyl]-4-{2-oxo-7-azaspiro[3.5]nonan-7-yl}benzamide (40 mg, 0.05 mmol, 1.0 eq), MeOH (1 mL), ZnCl₂ (20 mg, 0.1 mmol, 3.0 eq), NaBH₃CN (9 mg, 0.1 mmol, 3.0 eq). The resulting solution was stirred for 6 hours at 70° C. The resulting mixture was then quenched by the addition of water (20 mL), and extracted with dichloromethane/methanol (10:1, 2×30 mL). The combined organic layers was washed with brine (2×20 mL), dried over anhydrous Na₂SO₄. After filtration, the filtrate was concentrated under vacuum. The residue was purified by Prep-TLC (dichloromethane/methanol=15:1) to give 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-4-{2-[(2S)-2-(2-isopropoxyphenyl)-4-{[2-methyl-7-(morpholin-4-yl)furo[2,3-c]pyridin-4-yl]methyl}piperazin-1-yl]-7-azaspiro[3.5]nonan-7-yl}-N-[3-nitro-4-({[(1r,4r)-4-hydroxy-4-methylcyclohexyl]methyl}amino)benzenesulfonyl]benzamide (assumed) as a light yellow solid (12 mg, 19.6%). LC-MS (ESI, m/z) M+1: 1249. ¹H NMR (400 MHz, Methanol-d₄) δ 8.49 (d, J=2.2 Hz, 1H), 7.85 (d, J=8.8 Hz, 1H), 7.74 (d, J=4.8 Hz, 1H), 7.49 (dd, J=9.2, 2.4 Hz, 1H), 7.42 (d, J=7.8 Hz, 1H), 7.23 (t, J=7.8 Hz, 1H), 7.07 (d, J=3.4 Hz, 1H), 6.96-6.87 (m, 3H), 6.71-6.65 (m, 3H), 6.49 (s, 1H), 6.02 (d, J=3.4 Hz, 1H), 4.62-4.55 (m, 1H), 4.28 (d, J=7.6 Hz, 2H), 3.91 (d, J=13.6 Hz, 1H), 3.88-3.81 (m, 4H), 3.77-3.65 (m, 7H), 3.51-3.44 (m, 2H), 3.27-3.24 (m, 2H), 3.17-3.07 (m, 7H), 2.70-2.65 (m, 1H), 2.53-2.46 (m, 5H), 1.96 (br, 1H), 1.84 (d, J=13.8 Hz, 2H), 1.73 (d, J=12.4 Hz, 6H), 1.60-1.48 (m, 5H), 1.45-1.39 (m, 2H), 1.36-1.33 (m, 2H), 1.31-1.25 (m, 8H), 1.22-1.17 (m, 4H).

### Example 148 Preparation of 4-(2-((R)-4-((3,3-dimethyl-2,3-dihydro-[1,4]dioxino[2,3-b]pyridin-8-yl)methyl)-2-(o-tolyl)piperazin-1-yl)-7-azaspiro[3.5]nonan-7-yl)-N-((4-((((1r,4r)-4-hydroxy-4-methylcyclohexyl)methyl)amino)-3-nitrophenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide (assumed)

Synthesis of (R)-3,3-dimethyl-8-((3-(o-tolyl)piperazin-1-yl)methyl)-2,3-dihydro-[1,4]dioxino[2,3-b]pyridine (assumed): Into an 8 mL sealed tube were added 3,3-dimethyl-2H-[1,4]dioxino[2,3-b]pyridine-8-carbaldehyde (164 mg, 0.9 mmol, 1.0 eq), (2R)-2-(2-methylphenyl)piperazine (150 mg, 0.9 mmol, 1.0 eq), NaBH₃CN (160 mg, 2.6 mmol, 3.0 eq), MeOH (3 mL) and ZnCl₂ (464 mg, 3.4 mmol, 4.0 eq) at 25° C. The resulting mixture was stirred for overnight at 25° C. The reaction was quenched by the addition of water (2 mL), and extracted with CH₂Cl₂ (2×10 mL). The combined organic layers were washed with brine (20 mL), dried over anhydrous Na₂SO₄. After filtration, the filtrate was concentrated under vacuum. The residue was purified by Prep-TLC (dichloromethane/methanol=12:1) to give (R)-3,3-dimethyl-8-((3-(o-tolyl)piperazin-1-yl)methyl)-2,3-dihydro-[1,4]dioxino[2,3-b]pyridine as a colorless oil (80 mg, 26.6%). LC-MS (ESI, m/z) M+1: 354.

Synthesis of 4-(2-((R)-4-((3,3-dimethyl-2,3-dihydro-[1,4]dioxino[2,3-b]pyridin-8-yl)methyl)-2-(o-tolyl)piperazin-1-yl)-7-azaspiro[3.5]nonan-7-yl)-N-((4-((((1r,4r)-4-hydroxy-4-methylcyclohexyl)methyl)amino)-3-nitrophenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide (assumed): Into an 8-mL sealed-tube, were placed (R)-3,3-dimethyl-8-((3-(o-tolyl)piperazin-1-yl)methyl)-2,3-dihydro-[1,4]dioxino[2,3-b]pyridine (30 mg, 0.09 mmol, 1.0 eq), 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-N-[3-nitro-4-({[(1r,4r)-4-hydroxy-4-methylcyclohexyl]methyl}amino)benzenesulfonyl]-4-{2-oxo-7-azaspiro[3.5]nonan-7-yl}benzamide (69 mg, 0.09 mmol, 1.0 eq), MeOH (3 mL), ZnCl₂ (35 mg, 0.3 mmol, 3.0 eq) and NaBH₃CN (16 mg, 0.3 mmol, 3.0 eq). The resulting solution was stirred for 4 hours at 70° C. The resulting mixture was then quenched by the addition of water (20 mL), and extracted with dichloromethane/methanol (10:1, 2×30 mL). The combined organic layers was washed with brine (2×20 mL), dried over anhydrous Na₂SO₄. After filtration, the filtrate was concentrated under vacuum. The residue was purified by Prep-TLC (dichloromethane/methanol=15:1) to give 4-(2-((R)-4-((3,3-dimethyl-2,3-dihydro-[1,4]dioxino[2,3-b]pyridin-8-yl)methyl)-2-(o-tolyl)piperazin-1-yl)-7-azaspiro[3.5]nonan-7-yl)-N-((4-((((1r,4r)-4-hydroxy-4-methylcyclohexyl)methyl)amino)-3-nitrophenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide (assumed) as a light yellow solid (25 mg, 25.6%). LC-MS (ESI, m/z) M+1: 1152. ¹H NMR (400 MHz, DMSO-d₆) δ 12.75 (s, 1H), 11.24 (s, 1H), 8.56 (s, 1H), 8.38 (s, 1H), 7.78 (d, J=9.3 Hz, 1H), 7.70 (d, J=4.9 Hz, 1H), 7.40 (m, 1H), 7.10-7.08 (m, 4H), 6.96 (d, J=5.1 Hz, 1H), 6.88-6.78 (m, 4H), 6.44 (s, 1H), 6.05 (s, 1H), 4.23 (s, 2H), 4.07 (m, 2H), 3.92 (s, 2H), 3.82 (s, 2H), 3.75 (m, 1H), 3.52 (s, 2H), 3.09 (s, 4H), 2.91 (d, J=8.8 Hz, 2H), 2.82 (s, 1H), 2.30 (s, 3H), 2.22 (m, 2H), 2.08 (m, 2H), 1.72-1.61 (m, 5H), 1.55 (m, 3H), 1.37 (s, 2H), 1.29-1.25 (m, 16H), 1.13-1.10 (m, 5H).

### Example 149 Preparation of 4-(2-((S)-4-((5,6-dimethoxypyridin-3-yl)methyl)-2-(o-tolyl)piperazin-1-yl)-7-azaspiro[3.5]nonan-7-yl)-N-((4-((((1r,4r)-4-hydroxy-4-methylcyclohexyl)methyl)amino)-3-nitrophenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide

Synthesis of (S)-1-((5,6-dimethoxypyridin-3-yl)methyl)-3-(o-tolyl)piperazine: Into a 40 mL vial were added 5,6-dimethoxypyridine-3-carbaldehyde (100 mg, 0.6 mmol, 1.0 eq), (S)-2-(o-tolyl)piperazine (105 mg, 0.6 mmol, 1.0 eq), NaBH₃CN (1878 mg, 3.0 mmol, 5.0 eq), ZnCl₂ (196 mg, 3.0 mmol, 5.0 eq) and MeOH (2 mL). The resulting mixture was stirred for 4 hours at 25° C. The reaction was quenched by the addition of water (2 mL) and extracted with EtOAc (3×5 mL). The combined organic layers were washed with brine (5 mL), dried over anhydrous Na₂SO₄. After filtration, the filtrate was concentrated under vacuum. The crude residue was purified by Prep-TLC (CH₂Cl₂/MeOH=10:1) to give (S)-1-((5,6-dimethoxypyridin-3-yl)methyl)-3-(o-tolyl)piperazine as an off-white solid (80 mg, 40.8%).

Synthesis of 4-(2-((S)-4-((5,6-dimethoxypyridin-3-yl)methyl)-2-(o-tolyl)piperazin-1-yl)-7-azaspiro[3.5]nonan-7-yl)-N-((4-((((1r,4r)-4-hydroxy-4-methylcyclohexyl)methyl)amino)-3-nitrophenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide: Into an 8 mL vial were added (S)-1-((5,6-dimethoxypyridin-3-yl)methyl)-3-(o-tolyl)piperazine (40 mg, 0.1 mmol, 1.0 eq), 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-N-[3-nitro-4-({[(1r,4r)-4-hydroxy- 4-methylcyclohexyl]methyl}amino)benzenesulfonyl]-4-{2-oxo-7-azaspiro[3.5]nonan-7-yl}benzamide (99 mg, 0.1 mmol, 1.0 eq), NaBH₃CN (39 mg, 0.6 mmol, 5.0 eq), ZnCl₂ (83 mg, 0.6 mmol, 5.0 eq) and MeOH (2 mL). The resulting mixture was stirred for 4 hours at 70° C. The reaction was quenched by the addition of water (2 mL) and extracted with EtOAc (3×5 mL). The combined organic layers were washed with brine (5 mL), dried over anhydrous Na₂SO₄. After filtration, the filtrate was concentrated under vacuum. The crude residue was purified by Prep-TLC (CH₂Cl₂/MeOH=15:1) to give 4-(2-((S)-4-((5,6-dimethoxypyridin-3-yl)methyl)-2-(o-tolyl)piperazin-1-yl)-7-azaspiro[3.5]nonan-7-yl)-N-((4-((((1r,4r)-4-hydroxy-4-methylcyclohexyl)methyl)amino)-3-nitrophenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide (17 mg, 11.6%) as a yellow solid. LC-MS (ESI, m/z) M+1: 1126. ¹H NMR (400 MHz, Methanol-d₄) δ 8.46-8.45 (m, 1H), 7.56-7.55 (m, 1H), 7.47-7.45 (m, 1H), 7.26-7.24 (m, 2H), 7.11 (s, 1H), 7.14-7.11 (m, 3H), 7.05 (s, 1H), 6.65-6.64 (m, 1H), 6.46-6.42 (m, 1H), 6.37 (s, 1H), 5.98 (s, 1H), 4.32-4.28 (m, 2H), 4.01 (s, 3H), 3.89 (s, 3H), 3.59-3.35 (m, 4H), 3.21 (t, J=6.1 Hz, 4H), 3.15-2.87 (m, 8H), 2.47-2.24 (m, 7H), 1.86-1.84 (m, 6H), 1.77 (d, J=13.1 Hz, 8H), 1.28-1.26 (m, 6H), 1.25-0.99 (m, 3H).

### Example 150 Preparation of 4-(2-((S)-4-((2,3-dimethoxypyridin-4-yl)methyl)-2-(2-isopropoxyphenyl)piperazin-1-yl)-7-azaspiro[3.5]nonan-7-yl)-N-((4-((((1r,4r)-4-hydroxy-4-methylcyclohexyl)methyl)amino)-3-nitrophenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide (assumed)

Synthesis of (S)-1-((2,3-dimethoxypyridin-4-yl)methyl)-3-(2-isopropoxyphenyl)piperazine (assumed): Into a 40-mL sealed-tube, were placed (S)-2-(2-isopropoxyphenyl)piperazine (assumed) (150 mg, 0.5 mmol, 1.0 eq), 5-(methoxy-d3)-6-morpholinonicotinaldehyde (118 mg, 0.5 mmol, 1.1 eq), MeOH(4 mL), NaBH(OAc)₃ (307 mg, 1.4 mmol, 3.0 eq). The resulting solution was stirred for 2 hours at 25° C. The resulting mixture was then quenched by the addition of water (40 mL). The resulting solution was extracted with ethyl acetate (2×40 mL) and washed with brine (2×40 mL) and the organic layers combined. The mixture was dried over anhydrous Na₂SO₄. After filtration, the filtrate was concentrated under vacuum. The crude residue was purified onto a silica gel column and eluted with ethyl acetate/petroleum ether=1:2 to give (S)-1-((2,3-dimethoxypyridin-4-yl)methyl)-3-(2-isopropoxyphenyl)piperazine (assumed) as a light yellow solid (110 mg, 44.1%). LC-MS (ESI, m/z) M+1: 372.

Synthesis of 4-(2-((S)-4-((2,3-dimethoxypyridin-4-yl)methyl)-2-(2-isopropoxyphenyl)piperazin-1-yl)-7-azaspiro[3.5]nonan-7-yl)-N-((4-((((1r,4r)-4-hydroxy-4-methylcyclohexyl)methyl)amino)-3-nitrophenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide (assumed): Into an 8 mL vial, were added N-((4-((((1r,4r)-4-hydroxy-4-methylcyclohexyl)methyl)amino)-3-nitrophenyl)sulfonyl)-4-(2-oxo-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide (87 mg, 0.1 mmol, 0.7 eq), (S)-1-((2,3-dimethoxypyridin-4-yl)methyl)-3-(2-isopropoxyphenyl)piperazine (assumed) (45 mg, 0.1 mmol, 1.0 eq), NaBH₃CN (41 mg, 0.7 mmol, 5.0 eq), ZnCl₂ (90 mg, 0.7 mmol, 5.0 eq) and MeOH (2 mL). The resulting mixture was stirred for 5 hours at 70° C. The reaction was quenched by the addition of water (2 mL). The resulting mixture was extracted with CH₂Cl₂ (3×3 mL) and washed with brine (3 mL), dried over anhydrous Na₂SO₄. After filtration, the filtrate was concentrated under vacuum. The residue was purified by Prep-TLC (CH₂Cl₂/MeOH 20:1) to give 4-(2-((S)-4-((2,3-dimethoxypyridin-4-yl)methyl)-2-(2-isopropoxyphenyl)piperazin-1-yl)-7-azaspiro[3.5]nonan-7-yl)-N-((4-((((1r,4r)-4-hydroxy-4-methylcyclohexyl)methyl)amino)-3-nitrophenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide (assumed) as a yellow solid (15 mg, 15.9%). LC-MS (ESI, m/z) M+1: 1169. ¹H NMR (300 MHz, Chloroform-d) δ 12.37 (s, 1H), 8.75 (s, 3H), 8.46 (s, 1H), 8.40 (s, 1H), 8.11 (d, J=9.6 Hz, 2H), 7.80 (s, 2H), 7.17 (d, J=16.5 Hz, 3H), 6.98 (s, 1H), 6.85 (d, J=8.1 Hz, 3H), 6.71 (d, J=9.3 Hz, 3H), 6.62 (s, 1H), 6.43 (s, 2H), 6.11 (s, 2H), 4.61-4.40 (m, 6H), 4.10 (s, 1H), 3.96 (s, 3H), 3.58 (s, 2H), 3.51 (d, J=9.0 Hz, 3H), 3.38 (s, 3H), 3.21 (t, J=6.3 Hz, 3H), 3.06-3.01 (m, 13H), 1.75 (s, 2H), 1.51 (d, J=12.3 Hz, 3H), 1.37 (dd, J=14.7, 9.0 Hz, 9H), 0.89 (d, J=8.1 Hz, 3H).

### Example 151 Preparation of N-((4-((((1r,4r)-4-hydroxy-4-methylcyclohexyl)methyl)amino)-3-nitrophenyl)sulfonyl)-4-(2-((S)-2-(2-isopropoxyphenyl)-4-((3-methoxy-2-morpholinopyridin-4-yl)methyl)piperazin-1-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide (assumed)

Synthesis of (S)-4-(4-((3-(2-isopropoxyphenyl)piperazin-1-yl)methyl)-3-methoxypyridin-2-yl)morpholine (assumed): Into a 40-mL sealed-tube, were placed (S)-2-(2-isopropoxyphenyl)piperazine (assumed) (150 mg, 0.5 mmol, 1.0 eq), 3-methoxy-2-morpholinoisonicotinaldehyde (118 mg, 0.5 mmol, 1.1 eq), MeOH(4 mL), NaBH(OAc)₃ (307 mg, 1.4 mmol, 3.0 eq). The resulting solution was stirred for 5 hours at 25° C. The resulting mixture was then quenched by the addition of water (40 mL), and extracted with ethyl acetate (2×40 mL). The combined organic layers was washed with brine (2×40 mL), dried over anhydrous Na₂SO₄. After filtration, the filtrate was concentrated under vacuum. The crude residue was purified onto a silica gel column and eluted with ethyl acetate/petroleum ether=1:2 to give (S)-4-(4-((3-(2-isopropoxyphenyl)piperazin-1-yl)methyl)-3-methoxypyridin-2-yl)morpholine (assumed) as a light yellow solid (110 mg, 44.1%). LC-MS (ESI, m/z) M+1: 427.

Synthesis of N-((4-((((1r,4r)-4-hydroxy-4-methylcyclohexyl)methyl)amino)-3-nitrophenyl)sulfonyl)-4-(2-((S)-2-(2-isopropoxyphenyl)-4-((3-methoxy-2-morpholinopyridin-4-yl)methyl)piperazin-1-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide (assumed): Into an 8 mL vial, were added N-((4-((((1r,4r)-4-hydroxy-4-methylcyclohexyl)methyl)amino)-3-nitrophenyl)sulfonyl)-4-(2-oxo-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide (87 mg, 0.1 mmol, 0.7 eq), (S)-4-(4-((3-(2-isopropoxyphenyl)piperazin-1-yl)methyl)-3-methoxypyridin-2-yl)morpholine (45 mg, 0.1 mmol, 1.0 eq), NaBH₃CN (41 mg, 0.7 mmol, 5.0 eq), ZnCl₂ (90 mg, 0.7 mmol, 5.0 eq) and MeOH (2 mL). The resulting mixture was stirred for 5 hours at 70° C. The reaction was quenched by the addition of water (2 mL) and extracted with CH₂Cl₂ (3×3 mL) and washed with brine (3 mL), dried over anhydrous Na₂SO₄. After filtration, the filtrate was concentrated under vacuum. The residue was purified by Prep-TLC (CH₂Cl₂/MeOH=20:1) to give N-((4-((((1r,4r)-4-hydroxy-4-methylcyclohexyl)methyl)amino)-3-nitrophenyl)sulfonyl)-4-(2-((S)-2-(2-isopropoxyphenyl)-4-((3-methoxy-2-morpholinopyridin-4-yl)methyl)piperazin-1-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide (assumed) as a yellow solid (15 mg, 15.9%). LC-MS (ESI, m/z) M+1: 1224. ¹H NMR (300 MHz, Chloroform-d) δ 12.57 (s, 1H), 8.65 (s, 2H), 8.46 (s, 2H), 8.40 (s, 4H), 8.11 (d, J=9.6 Hz, 2H), 7.80 (s, 2H), 7.07 (d, J=16.5 Hz, 3H), 6.98 (s, 1H), 6.85 (d, J=8.1 Hz, 4H), 6.71 (d, J=9.3 Hz, 2H), 6.62 (s, 1H), 6.43 (s, 2H), 6.11 (s, 2H), 4.61-4.50 (m, 1H), 4.00 (s, 1H), 3.87 (s, 9H), 3.58 (s, 2H), 3.51 (d, J=9.0 Hz, 4H), 3.38-3.25 (m, 7H), 3.21 (t, J=6.3 Hz, 3H), 3.06-2.96 (m, 10H), 1.75 (s, 3H), 1.51 (d, J=12.3 Hz, 1H), 1.37 (dd, J=14.7, 9.0 Hz, 9H), 0.89 (d, J=8.1 Hz, 3H).

### Example 152 Preparation of 4-(2-((S)-4-((2,3-dihydrobenzo[b][1,4]dioxin-6-yl)methyl)-2-(2-isopropoxyphenyl)piperazin-1-yl)-7-azaspiro[3.5]nonan-7-yl)-N-((4-((((1r,4r)-4-hydroxy-4-methylcyclohexyl)methyl)amino)-3-nitrophenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide (assumed)

Synthesis of (S)-1-((2,3-dihydrobenzo[b][1,4]dioxin-6-yl)methyl)-3-(2-isopropoxyphenyl)piperazine (assumed): Into an 8 mL sealed tube were added 2,3-dihydrobenzo[b][1,4]dioxine-6-carbaldehyde (112 mg, 0.5 mmol, 0.8 eq), (2S)-2-(2-isopropoxyphenyl)piperazine (assumed) (150 mg, 0.7 mmol, 1.0 eq), NaBH₃CN (128 mg, 2.0 mmol, 3.0 eq), MeOH (3 mL) and ZnCl₂ (278 mg, 2.0 mmol, 3.0 eq) at 25° C. The resulting mixture was stirred for 16 hours at 25° C. The reaction was quenched by the addition of water (2 mL) and extracted with CH₂Cl₂ (2×10 mL). The combined organic layers were washed with brine (20 mL), dried over anhydrous Na₂SO₄. After filtration, the filtrate was concentrated under vacuum. The residue was purified by Prep-TLC (dichloromethane/methanol=12:1) to give (S)-1-((2,3-dihydrobenzo[b][1,4]dioxin-6-yl)methyl)-3-(2-isopropoxyphenyl)piperazine (assumed) as a colorless oil (105 mg, 41.8%). LC-MS (ESI, m/z) M+1: 369.

Synthesis of 4-(2-((S)-4-((2,3-dihydrobenzo[b][1,4]dioxin-6-yl)methyl)-2-(2-isopropoxyphenyl)piperazin-1-yl)-7-azaspiro[3.5]nonan-7-yl)-N-((4-((((1r,4r)-4-hydroxy-4-methylcyclohexyl)methyl)amino)-3-nitrophenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide (assumed): Into an 8-mL sealed-tube, were placed (S)-1-((2,3-dihydrobenzo[b][1,4]dioxin-6-yl)methyl)-3-(2-isopropoxyphenyl)piperazine (assumed) (18 mg, 0.05 mmol, 1.0 eq), N-((4-((((1r,4r)-4-hydroxy-4-methylcyclohexyl)methyl)amino)-3-nitrophenyl)sulfonyl)-4-(2-oxo-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide (40 mg, 0.05 mmol, 1.0 eq), MeOH (3 mL), ZnCl₂ (20 mg, 0.15 mmol, 3.0 eq) and NaBH₃CN (9 mg, 0.15 mmol, 3.0 eq). The resulting solution was stirred for 4 hours at 70° C. The resulting mixture was then quenched by the addition of water (20 mL) and extracted with dichloromethane/methanol (10:1, 2×30 mL). The combined organic layers was washed with brine (2×20 mL) and dried over anhydrous Na₂SO₄. After filtration, the filtrate was concentrated under vacuum. The residue was purified by Prep-TLC (dichloromethane/methanol=15:1) to give 4-(2-((S)-4-((2,3-dihydrobenzo[b][1,4]dioxin-6-yl)methyl)-2-(2-isopropoxyphenyl)piperazin-1-yl)-7-azaspiro[3.5]nonan-7-yl)-N-((4-((((1r,4r)-4-hydroxy-4-methylcyclohexyl)methyl)amino)-3-nitrophenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide (assumed) as a light yellow solid (25 mg, 43.6%). LC-MS (ESI, m/z) M+1: 1166. ¹H NMR (300 MHz, DMSO-d₆) δ 12.76 (s, 1H), 11.24 (s, 1H), 8.57 (s, 1H), 8.38-8.35 (m, 1H), 7.77 (s, 1H), 7.58 (s, 1H), 7.38-7.35 (m, 2H), 7.14-7.10 (m, 2H), 6.92-6.88 (m, 2H), 6.84-6.80 (m, 2H), 6.75-6.70 (m, 2H), 6.43 (s, 1H), 6.04 (s, 1H), 4.55 (s, 1H), 4.25-4.20 (m, 6H), 4.09 (s, 1H), 3.83-3.80 (m, 2H), 3.70 (s, 1H), 3.52-3.48 (m, 1H), 3.28-3.25 (m, 3H), 3.13-3.10 (m, 4H), 3.03-2.98 (m, 2H), 2.89-2.85 (m, 5H), 2.15-2.10 (m, 2H), 1.88 (s, 1H), 1.68-1.65 (m, 4H), 1.57-1.53 (m, 3H), 1.33-1.30 (m, 10H), 1.27-1.13 (m, 6H), 1.10 (s, 3H), 0.85 (d, J=7.2 Hz, 1H).

### Example 153 Preparation of 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-4-{2-[(2S)-4-({3-fluoro-4-[(3R)-3-methylmorpholin-4-yl]phenyl}methyl)-2-(2-isopropoxyphenyl)piperazin-1-yl]-7-azaspiro[3.5]nonan-7-yl}-N-[3-nitro-4-({[(1r,4r)-4-hydroxy-4-methylcyclohexyl]methyl}amino)benzenesulfonyl]benzamide (assumed)

Synthesis of (3R)-4-(2-fluoro-4-{[(3S)-3-(2-isopropoxyphenyl)piperazin-1-yl]methyl}phenyl)-3-methylmorpholine (assumed): Into an 8-mL sealed tube, were placed 3-fluoro-4-[(3R)-3-methylmorpholin-4-yl]benzaldehyde (100 mg, 0.4 mmol, 1.0 eq), (2S)-2-(2-isopropoxyphenyl)piperazine (assumed) (99 mg, 0.4 mmol, 1.0 eq), NaBH(OAc)₃ (285 mg, 1.3 mmol, 3.0 eq), DCE (1 mL). The resulting solution was stirred for 1.5 hours at 25° C. The resulting mixture was then quenched by the addition of water (30 mL) and extracted with dichloromethane (2×30 mL). The combined organic layers was washed with brine (2×30 mL), dried over anhydrous Na₂SO₄. After filtration, the filtrate was concentrated under vacuum. The crude residue was applied onto a silica gel column and eluted with dichloromethane/methanol=10:1 to give (3R)-4-(2-fluoro-4-{[(3S)-3-(2-isopropoxyphenyl)piperazin-1-yl]methyl}phenyl)-3-methylmorpholine (assumed) as a yellow solid (120 mg, 62.7%). LC-MS (ESI, m/z) M+1: 428.

Synthesis of 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-4-{2-[(2S)-4-({3-fluoro-4-[(3R)-3-methylmorpholin-4-yl]phenyl}methyl)-2-(2-isopropoxyphenyl)piperazin-1-yl]-7-azaspiro[3.5]nonan-7-yl}-N-[3-nitro-4-({[(1r,4r)-4-hydroxy-4-methylcyclohexyl]methyl}amino)benzenesulfonyl]benzamide (assumed): Into an 8-mL sealed tube, were placed (3R)-4-(2-fluoro-4-{[(3S)-3-(2-isopropoxyphenyl)piperazin-1-yl]methyl}phenyl)-3-methylmorpholine (assumed) (42 mg, 0.09 mmol, 1.0 eq), 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-N-[3-nitro-4-({[(1r,4r)-4-hydroxy- 4-methylcyclohexyl]methyl}amino)benzenesulfonyl]-4-{2-oxo-7-azaspiro[3.5]nonan-7-yl}benzamide (80 mg, 0.1 mmol, 1.0 eq), ZnCl₂ (40 mg, 0.3 mmol, 3.0 eq), NaBH₃CN (19 mg, 0.3 mmol, 3.0 eq), MeOH (1 mL). The resulting solution was stirred for 4 hours at 70° C. The resulting mixture was then quenched by the addition of water (30 mL), and extracted with dichloromethane (2×30 mL). The combined organic layers was washed with brine (2×30 mL), dried over anhydrous Na₂SO₄. After filtration, the filtrate was concentrated under vacuum. The crude residue was applied onto a silica gel column and eluted with dichloromethane/methanol=17:1 to give 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-4-{2-[(2S)-4-({3-fluoro-4-[(3R)-3-methylmorpholin-4-yl]phenyl}methyl)-2-(2-isopropoxyphenyl)piperazin-1-yl]-7-azaspiro[3.5]nonan-7-yl}-N-[3-nitro-4-({[(1r,4r)-4-hydroxy-4-methylcyclohexyl]methyl}amino)benzenesulfonyl]benzamide (assumed) as a yellow solid (20 mg, 16.6%). LC-MS (ESI, m/z) M+1: 1225. ¹H NMR (400 MHz, Methanol-d₄) δ 8.54-8.45 (m, 1H), 7.90-7.62 (m, 1H), 7.54-7.39 (m, 2H), 7.33-7.18 (m, 1H), 7.14-7.04 (m, 4H), 6.97-6.84 (m, 3H), 6.73-6.56 (m, 2H), 6.55-6.47 (m, 1H), 6.04-5.91 (m, 1H), 4.74-4.54 (m, 1H), 4.36-4.02 (m, 3H), 3.98-3.75 (m, 4H), 3.70 (s, 1H), 3.63-3.39 (m, 6H), 3.29-3.21 (m, 2H), 3.20-3.03 (m, 7H), 3.00-2.94 (m, 1H), 2.90-2.82 (m, 1H), 2.71-2.64 (m, 1H), 2.54-2.20 (m, 3H), 1.95 (s, 1H), 1.88-1.80 (m, 2H), 1.78-1.64 (m, 4H), 1.60-1.47 (m, 4H), 1.45-1.36 (m, 4H), 1.35-1.29 (m, 4H), 1.28-1.23 (m, 9H), 0.93-0.86 (m, 3H).

### Example 154 Preparation of 4-{2-[(2R)-4-[(3,4-difluorophenyl)methyl]-2-(2-methylphenyl)piperazin-1-yl]-7-azaspiro[3.5]nonan-7-yl}-2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-N-[3-nitro-4-({[(1r,4r)-4-hydroxy-4-methylcyclohexyl]methyl}amino)benzenesulfonyl]benzamide

Synthesis of (3R)-1-[(3,4-difluorophenyl)methyl]-3-(2-methylphenyl)piperazine: Into an 8-mL sealed tube, were placed 3,4-difluorobenzaldehyde (200 mg, 1.4 mmol, 1.0 eq), (2R)-2-(2-methylphenyl)piperazine (248 mg, 1.4 mmol, 1.0 eq), NaBH₃CN (265 mg, 4.2 mmol, 3.0 eq), ZnCl₂ (575 mg, 4.2 mmol, 3.0 eq), MeOH (2 mL). The resulting solution was stirred for 2 hours at 25° C. The resulting mixture was then quenched by the addition of water (30 mL), and extracted with dichloromethane (2×30 mL). The combined organic layers was washed with brine (2×30 mL), dried over anhydrous Na₂SO₄. After filtration, the filtrate was concentrated under vacuum. The crude residue was applied onto a silica gel column and eluted with dichloromethane/methanol=10:1 to give (3R)-1-[(3,4-difluorophenyl)methyl]-3-(2-methylphenyl)piperazine as a white solid (260 mg, 61.1%). LC-MS (ESI, m/z) M+1: 303.

Synthesis of 4-{2-[(2R)-4-[(3,4-difluorophenyl)methyl]-2-(2-methylphenyl)piperazin-1-yl]-7-azaspiro[3.5]nonan-7-yl}-2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-N-[3-nitro-4-({[(1r,4r)-4-hydroxy-4-methylcyclohexyl]methyl}amino)benzenesulfonyl]benzamide: Into an 8-mL sealed tube, were placed (3R)-1-[(3,4-difluorophenyl)methyl]-3-(2-methylphenyl)piperazine (30 mg, 0.1 mmol, 1.0 eq), 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-N-[3-nitro-4-({[(1r,4r)-4-hydroxy-4-methylcyclohexyl]methyl}amino)benzenesulfonyl]-4-{2-oxo-7-azaspiro[3.5]nonan-7-yl}benzamide (80 mg, 0.1 mmol, 1.0 eq), ZnCl₂ (40 mg, 0.3 mmol, 3.0 eq), NaBH₃CN (19 mg, 0.3 mmol, 3.0 eq), MeOH (1 mL). The resulting solution was stirred for 4 hours at 70° C. The resulting mixture was then quenched by the addition of water (30 mL), and extracted with dichloromethane (2×30 mL). The combined organic layers was washed with brine (2×30 mL), dried over anhydrous Na₂SO₄. After filtration, the filtrate was concentrated under vacuum. The crude residue was applied onto a silica gel column and eluted with dichloromethane/methanol=15:1 to give 4-{2-[(2R)-4-[(3,4-difluorophenyl)methyl]-2-(2-methylphenyl)piperazin-1-yl]-7-azaspiro[3.5]nonan-7-yl}-2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-N-[3-nitro-4-({[(1r,4r)-4-hydroxy-4-methylcyclohexyl]methyl}amino)benzenesulfonyl]benzamide as a yellow solid (20 mg, 18.5%). LC-MS (ESI, m/z) M+1: 1100. ¹H NMR (400 MHz, Methanol-d₄) δ 8.53-8.46 (m, 1H), 7.89-7.60 (m, 1H), 7.53-7.39 (m, 2H), 7.37-7.20 (m, 3H), 7.19-7.09 (m, 4H), 7.08-7.03 (m, 1H), 6.99-6.86 (m, 1H), 6.72-6.63 (m, 1H), 6.57-6.45 (m, 1H), 6.05-5.93 (m, 1H), 4.44-4.21 (m, 2H), 4.17-3.81 (m, 1H), 3.75-3.42 (m, 6H), 3.29-3.22 (m, 2H), 3.19-2.86 (m, 7H), 2.70-2.59 (m, 1H), 2.40-2.22 (m, 6H), 1.93-1.81 (m, 3H), 1.79-1.63 (m, 4H), 1.62-1.39 (m, 6H), 1.36-1.22 (m, 9H).

### Example 155 Preparation of 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-4-{2-[(2R)-4-({3-fluoro-4-[(3R)-3-methylmorpholin-4-yl]phenyl}methyl)-2-(2-methylphenyl)piperazin-1-yl]-7-azaspiro[3.5]nonan-7-yl}-N-[3-nitro-4-({[(1r,4r)-4-hydroxy-4-methylcyclohexyl]methyl}amino)benzenesulfonyl]benzamide

Synthesis of (3R)-4-(2-fluoro-4-{[(3R)-3-(2-methylphenyl)piperazin-1-yl]methyl}phenyl)-3-methylmorpholine: Into an 8-mL sealed tube, were placed 3-fluoro-4-[(3R)-3-methylmorpholin-4-yl]benzaldehyde (200 mg, 0.9 mmol, 1.0 eq), (2R)-2-(2-methylphenyl)piperazine (158 mg, 0.9 mmol, 1.0 eq), NaBH₃CN (169 mg, 2.7 mmol, 3.0 eq), ZnCl₂ (366 mg, 2.7 mmol, 3.0 eq), MeOH (2 mL). The resulting solution was stirred for 1.5 hours at 25° C. The resulting mixture was then quenched by the addition of water (30 mL), and extracted with dichloromethane (2×30 mL). The combined organic layers was washed with brine (2×30 mL), dried over anhydrous Na₂SO₄. After filtration, the filtrate was concentrated under vacuum. The crude residue was applied onto a silica gel column and eluted with dichloromethane/methanol=10:1 to give (3R)-4-(2-fluoro-4-{[(3R)-3-(2-methylphenyl)piperazin-1-yl]methyl}phenyl)-3-methylmorpholine as a white solid (250 mg, 72.8%). LC-MS (ESI, m/z) M+1: 384.

Synthesis of 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-4-{2-[(2R)-4-({3-fluoro-4-[(3R)-3-methylmorpholin-4-yl]phenyl}methyl)-2-(2-methylphenyl)piperazin-1-yl]-7-azaspiro[3.5]nonan-7-yl}-N-[3-nitro-4-({[(1r,4r)-4-hydroxy-4-methylcyclohexyl]methyl}amino)benzenesulfonyl]benzamide: Into an 8-mL sealed tube, were placed (3R)-4-(2-fluoro-4-{[(3R)-3-(2-methylphenyl)piperazin-1-yl]methyl}phenyl)-3-methylmorpholine (38 mg, 0.1 mmol, 1.0 eq), 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-N-[3-nitro-4-({[(1r,4r)-4-hydroxy-4-methylcyclohexyl]methyl}amino)benzenesulfonyl]-4-{2-oxo-7-azaspiro[3.5]nonan-7-yl}benzamide (80 mg, 0.1 mmol, 1.0 eq), ZnCl₂ (40 mg, 0.3 mmol, 3.0 eq), NaBH₃CN (19 mg, 0.3 mmol, 3.0 eq), MeOH (1 mL). The resulting solution was stirred for 4 hours at 70° C. The resulting mixture was then quenched by the addition of water (30 mL), and then extracted with dichloromethane (2×30 mL). The combined organic layers was washed with brine (2×30 mL) and the organic layers combined. The mixture was dried over anhydrous Na₂SO₄. After filtration, the filtrate was concentrated under vacuum. The crude residue was applied onto a silica gel column and eluted with dichloromethane/methanol=15:1 to give 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-4-{2-[(2R)-4-({3-fluoro-4-[(3R)-3-methylmorpholin-4-yl]phenyl}methyl)-2-(2-methylphenyl)piperazin-1-yl]-7-azaspiro[3.5]nonan-7-yl}-N-[3-nitro-4-({[(1r,4r)-4-hydroxy-4-methylcyclohexyl]methyl}amino)benzenesulfonyl]benzamide as a yellow solid (20 mg, 17.2%). LC-MS (ESI, m/z) M+1: 1182. ¹H NMR (400 MHz, Methanol-d₄) δ 8.49-8.46 (m, 1H), 7.92-7.60 (m, 1H), 7.56-7.39 (m, 2H), 7.18-7.05 (m, 7H), 6.98-6.86 (m, 1H), 6.72-6.57 (m, 2H), 6.53-6.45 (m, 1H), 6.04-5.93 (m, 1H), 4.35-4.09 (m, 2H), 3.95-3.67 (m, 6H), 3.62-3.55 (m, 1H), 3.54-3.43 (m, 4H), 3.30-3.22 (m, 2H), 3.20-3.01 (m, 7H), 2.99-2.93 (m, 1H), 2.92-2.83 (m, 1H), 2.78-2.64 (m, 1H), 2.45-2.26 (m, 6H), 1.94-1.79 (m, 3H), 1.77-1.63 (m, 4H), 1.60-1.21 (m, 15H), 1.18-1.07 (m, 1H), 0.97-0.84 (m, 3H).

### Example 156 Preparation of 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-4-{2-[(2R)-2-(2-methylphenyl)-4-(oxan-4-ylmethyl)piperazin-1-yl]-7-azaspiro[3.5]nonan-7-yl}-N-[3-nitro-4-({[(1r,4r)-4-hydroxy-4-methylcyclohexyl]methyl}amino)benzenesulfonyl]benzamide

Synthesis of (3R)-3-(2-methylphenyl)-1-(oxan-4-ylmethyl)piperazine: Into an 8 mL vial were, added (2R)-2-(2-methylphenyl)piperazine (70 mg, 0.4 mmol, 1.0 eq), DCE (5 mL), oxane-4-carbaldehyde (45 mg, 0.4 mmol, 1.0 eq), NaBH(OAc)₃ (252 mg, 1.2 mmol, 3.0 eq). The resulting mixture was stirred for 4 hours at 25° C. The reaction was quenched with water (5 mL) and extracted with dichloromethane (3×10 mL). The combined organic layers were dried over anhydrous Na₂SO₄. After filtration, the filtrate was concentrated under vacuum. The residue was purified by Prep-TLC dichloromethane/methanol=10:1 to give (3R)-3-(2-methylphenyl)-1-(oxan-4-ylmethyl)piperazine (assumed) as a colorless oil (35 mg, 32.1%). LC-MS (ESI, m/z) M+1: 275.

Synthesis of 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-4-{2-[(2R)-2-(2-methylphenyl)-4-(oxan-4-ylmethyl)piperazin-1-yl]-7-azaspiro[3.5]nonan-7-yl}-N-[3-nitro-4-({[(1r,4r)-4-hydroxy-4-methylcyclohexyl]methyl}amino)benzenesulfonyl]benzamide: Into an 8 mL vial, were added (3R)-3-(2-methylphenyl)-1-(oxan-4-ylmethyl)piperazine (25 mg, 0.1 mmol, 1.0 eq), MeOH (2 mL), 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-N-[3-nitro-4-({[(1r,4r)-4-hydroxy-4-methylcyclohexyl]methyl}amino)benzenesulfonyl]-4-{2-oxo-7-azaspiro[3.5]nonan-7-yl}benzamide (75 mg, 0.1 mmol, 1.0 eq), ZnCl₂ (38 mg, 0.3 mmol, 3.0 eq) and NaBH₃CN (17 mg, 0.3 mmol, 3.0 eq). The resulting mixture was stirred for 4 hours at 70° C. The reaction was quenched by the addition of water (2 mL) and extracted with dichloromethane (3×4 mL). The combined organic layers were dried over anhydrous Na₂SO₄. After filtration, the filtrate was concentrated under vacuum. The crude residue was purified by Prep-TLC (dichloromethane/methanol=15:1) to give 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-4-{2-[(2R)-2-(2-methylphenyl)-4-(oxan-4-ylmethyl)piperazin-1-yl]-7-azaspiro[3.5]nonan-7-yl}-N-[3-nitro-4-({[(1r,4r)-4-hydroxy-4-methylcyclohexyl]methyl}amino)benzenesulfonyl]benzamide as a yellow solid (24 mg, 23.1%). LC-MS (ESI, m/z) M+1: 1072. ¹H NMR (400 MHz, Methanol-d₄) δ 8.48 (d, J=2.2 Hz, 1H), 7.87 (d, J=9.3 Hz, 1H), 7.52-7.42 (m, 2H), 7.23-7.11 (m, 3H), 7.08 (d, J=3.5 Hz, 1H), 7.00-6.88 (m, 1H), 6.71-6.65 (m, 2H), 6.49 (s, 1H), 6.02 (d, J=3.5 Hz, 1H), 4.29 (d, J=7.1 Hz, 2H), 3.92 (d, J=11.6 Hz, 3H), 3.76-3.62 (m, 2H), 3.55-3.34 (m, 4H), 3.26 (d, J=6.6 Hz, 2H), 3.19-2.97 (m, 9H), 2.72 (d, J=11.6 Hz, 1H), 2.47-2.18 (m, 6H), 1.99-1.61 (m, 10H), 1.60-1.22 (m, 13H), 1.15 (s, 2H), 0.91 (d, J=8.8 Hz, 2H).

### Example 157 Preparation of 4-(2-((S)-4-(4-cyclopropyl-3-methoxybenzyl)-2-(2-isopropoxyphenyl)piperazin-1-yl)-7-azaspiro[3.5]nonan-7-yl)-N-((4-((((1r,4r)-4-hydroxy-4-methylcyclohexyl)methyl)amino)-3-nitrophenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide (assumed)

Synthesis of (S)-1-(4-cyclopropyl-3-methoxybenzyl)-3-(2-isopropoxyphenyl)piperazine (assumed): Into a 40 mL vial were added 4-cyclopropyl-3-methoxybenzaldehyde (80 mg, 0.5 mmol, 1.0 eq), (S)-2-(2-isopropoxyphenyl)piperazine (assumed) (100 mg, 0.5 mmol, 1.0 eq), NaBH(OAc)₃ (481 mg, 2.3 mmol, 5.0 eq) and DCE (1 mL, 10.0 eq). The reaction mixture was stirred for 12 hours at 60° C. The residue was dissolved in CH₂Cl₂ (5 mL). The crude residue was purified by Prep-TLC (CH₂Cl₂/MeOH 10:1) to give (S)-1-(4-cyclopropyl-3-methoxybenzyl)-3-(2-isopropoxyphenyl)piperazine (assumed) as a light yellow solid (54 mg, 32.4%). LC-MS (ESI, m/z) M+1: 381.

Synthesis of 4-(2-((S)-4-(4-cyclopropyl-3-methoxybenzyl)-2-(2-isopropoxyphenyl)piperazin-1-yl)-7-azaspiro[3.5]nonan-7-yl)-N-((4-((((1r,4r)-4-hydroxy-4-methylcyclohexyl)methyl)amino)-3-nitrophenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide (assumed): Into an 8 mL vial were added (S)-1-(4-cyclopropyl-3-methoxybenzyl)-3-(2-isopropoxyphenyl)piperazine (assumed) (16 mg, 0.1 mmol, 1.0 eq), 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-N-[3-nitro-4-({[(1r,4r)-4-hydroxy-4-methylcyclohexyl]methyl}amino)benzenesulfonyl]-4-{2-oxo-7-azaspiro[3.5]nonan-7-yl}benzamide (35 mg, 0.1 mmol, 1.0 eq), NaBH₃CN (13 mg, 0.2 mmol, 5.0 eq), ZnCl₂ (29 mg, 0.2 mmol, 5.0 eq) and MeOH (1 mL, 10.0 eq). The resulting mixture was stirred for additional 4 hours at 70° C. The reaction was quenched by the addition of water (10 mL) and extracted with CH₂Cl₂ (3×5 mL). The combined organic layers were washed with brine (10 mL), dried over anhydrous Na₂SO₄. After filtration, the filtrate was concentrated under vacuum. The crude residue was purified by Prep-TLC (CH₂Cl₂/MeOH=18:1) to give 4-(2-((S)-4-(4-cyclopropyl-3-methoxybenzyl)-2-(2-isopropoxyphenyl)piperazin-1-yl)-7-azaspiro[3.5]nonan-7-yl)-N-((4-((((1r,4r)-4-hydroxy-4-methylcyclohexyl)methyl)amino)-3-nitrophenyl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide (assumed) as a yellow solid (10 mg, 19.8%). LC-MS (ESI, m/z) M+1: 1179. ¹H NMR (300 MHz, Chloroform-d) δ 12.58 (s, 1H), 8.64 (s, 1H), 8.47 (s, 1H), 8.37 (s, 1H), 8.12 (d, J=8.1 Hz, 2H), 7.81 (d, J=9.3 Hz, 1H), 7.49 (s, 1H), 7.08 (d, J=15.8 Hz, 2H), 6.83 (d, J=10.4 Hz, 2H), 6.71 (d, J=9.4 Hz, 2H), 6.62 (s, 1H), 6.43 (s, 1H), 6.11 (s, 1H), 4.71 (s, 6H), 4.20 (s, 4H), 3.92-3.90 (m, 6H), 3.72-3.37 (m, 7H), 3.21-3.20 (m, 4H), 3.06 (s, 4H), 2.64 (s, 2H), 2.18 (s, 7H), 1.82-1.80 (m, 7H), 1.42 (s, 3H), 1.37 (s, 5H), 1.28 (s, 6H), 1.21 (s, 1H).

### Example 158 Preparation of N-((4-((((1r,4r)-4-hydroxy-4-methylcyclohexyl)methyl)amino)-3-nitrophenyl)sulfonyl)-4-(2-((S)-2-(2-isopropoxyphenyl)-4-((6-methoxy-5-methylpyridin-3-yl)methyl)piperazin-1-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide (assumed)

Synthesis of (S)-3-(2-isopropoxyphenyl)-1-((6-methoxy-5-methylpyridin-3-yl)methyl)piperazine (assumed): Into a 40 mL sealed tube were added 6-methoxy-5-methylnicotinaldehyde (70 mg, 0.5 mmol, 1.0 eq), (2S)-2-(2-isopropoxyphenyl)piperazine (assumed) (100 mg, 0.5 mmol, 1.0 eq), NaBH₃CN (141 mg, 2.3 mmol, 5.0 eq), ZnCl₂ (306 mg, 2.3 mmol, 5.0 eq) and MeOH (1 mL, 10 eq). The reaction mixture was stirred for 4 hours at 25° C. The crude residue was purified by Prep-TLC (CH₂Cl₂/MeOH=10:1) to give (S)-3-(2-isopropoxyphenyl)-1-((6-methoxy-5-methylpyridin-3-yl)methyl)piperazine (assumed) as a white solid (92 mg, 57.5%). LC-MS (ESI, m/z) M+1: 356.

Synthesis of N-((4-((((1r,4r)-4-hydroxy-4-methylcyclohexyl)methyl)amino)-3-nitrophenyl)sulfonyl)-4-(2-((S)-2-(2-isopropoxyphenyl)-4-((6-methoxy-5-methylpyridin-3-yl)methyl)piperazin-1-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide (assumed): Into an 8 mL vial were added (S)-3-(2-isopropoxyphenyl)-1-((6-methoxy-5-methylpyridin-3-yl)methyl)piperazine (assumed) (16 mg, 0.1 mmol, 1.0 eq), 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-N-[3-nitro-4-({[(1r,4r)-4-hydroxy-4-methylcyclohexyl]methyl}amino)benzenesulfonyl]-4-{2-oxo-7-azaspiro[3.5]nonan-7-yl}benzamide (35 mg, 0.1 mmol, 1.0 eq), NaBH₃CN (14 mg, 0.2 mmol, 5.0 eq), ZnCl₂ (29 mg, 0.2 mmol, 5.0 eq) and MeOH (1 mL, 10.0 eq). The resulting mixture was stirred for additional 4 hours at 70° C. The resulting mixture was diluted with CH₂Cl₂ (3×5 mL), and then washed with brine (3×10 mL). The organic layer was dried over anhydrous Na₂SO₄. After filtration, the filtrate was concentrated under vacuum. The crude residue was purified by Prep-TLC (CH₂Cl₂/MeOH=18:1) to give N-((4-((((1r,4r)-4-hydroxy-4-methylcyclohexyl)methyl)amino)-3-nitrophenyl)sulfonyl)-4-(2-((S)-2-(2-isopropoxyphenyl)-4-((6-methoxy-5-methylpyridin-3-yl)methyl)piperazin-1-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide) (assumed) as a yellow solid (12 mg, 24.3%). LC-MS (ESI, m/z) M+1: 1154. ¹H NMR (300 MHz, Chloroform-d) δ 12.58 (s, 1H), 8.64 (d, J=2.3 Hz, 1H), 8.47 (s, 1H), 8.36 (s, 1H), 8.13 (s, 1H), 7.89 (s, 1H), 7.81 (d, J=10.2 Hz, 1H), 7.63 (s, 1H), 7.35 (s, 1H), 7.14-7.01 (m, 2H), 6.97-6.79 (m, 2H), 6.71 (d, J=9.3 Hz, 1H), 6.62 (s, 1H), 6.42 (s, 1H), 6.11 (s, 1H), 4.71-4.35 (m, 5H), 3.94 (s, 3H), 3.65-3.15 (m, 9H), 3.06 (s, 4H), 2.78 (s, 2H), 2.64 (s, 2H), 2.18 (s, 3H), 1.92-1.77 (m, 3H), 1.75 (s, 2H), 1.57-1.54 (m, 13H), 1.37 (d, J=6.1 Hz, 3H), 1.30-1.29 (m, 5H), 1.21 (s, 1H), 0.90 (s, 1H).

### Example 159 Preparation of 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-4-{2-[(2S)-2-(2-isopropoxyphenyl)-4-(oxan-4-ylmethyl)piperazin-1-yl]-7-azaspiro[3.5]nonan-7-yl}-N-[3-nitro-4-({[(1r,4r)-4-hydroxy-4-methylcyclohexyl]methyl}amino)benzenesulfonyl]benzamide (assumed)

Synthesis of (S)-3-(2-isopropoxyphenyl)-1-((tetrahydro-2H-pyran-4-yl)methyl)piperazine (assumed): Into a 40 mL vial were added (S)-2-(2-isopropoxyphenyl)piperazine (assumed) (100 mg, 0.5 mmol, 1.0 eq), tetrahydro-2H-pyran-4-carbaldehyde (52 mg, 0.4 mmol, 1.0 eq), NaBH₃CN (142 mg, 2.3 mmol, 5.0 eq), ZnCl₂ (309 mg, 2.3 mmol, 5.0 eq) and MeOH (1 mL, 10.0 eq). The reaction mixture was stirred for 3 hours at 25° C. The crude residue was purified by Prep-TLC (CH₂Cl₂/MeOH=15:1) to give (S)-3-(2-isopropoxyphenyl)-1-((tetrahydro-2H-pyran-4-yl)methyl)piperazine (assumed) as a white solid (120 mg, 83.1%). LC-MS (ESI, m/z) M+1: 319.

Synthesis of 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-4-{2-[(2S)-2-(2-isopropoxyphenyl)-4-(oxan-4-ylmethyl)piperazin-1-yl]-7-azaspiro[3.5]nonan-7-yl}-N-[3-nitro-4-({[(1r,4r)-4-hydroxy-4-methylcyclohexyl]methyl}amino)benzenesulfonyl]benzamide (assumed): Into an 8 mL vial were added (3S)-3-(2-isopropoxyphenyl)-1-(oxan-4-ylmethyl)piperazine (assumed) (13.6 mg, 0.043 mmol, 1 eq), 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-N-[3-nitro-4-({[(1r,4r)-4-hydroxy-4-methylcyclohexyl]methyl}amino)benzenesulfonyl]-4-{2-oxo-7-azaspiro[3.5]nonan-7-yl}benzamide (35 mg, 0.1 mmol, 1.0 eq), NaBH₃CN (13 mg, 0.2 mmol, 5.0 eq), ZnCl₂ (29 mg, 0.2 mmol, 5.0 eq) and MeOH (1 mL, 10.0 eq). The resulting mixture was stirred for additional 4 h at 70° C. The resulting mixture was diluted with CH₂Cl₂ (3×6 mL), and then washed with brine (3×2 mL). The organic layer was dried over anhydrous Na₂SO₄. After filtration, the filtrate was concentrated under vacuum. The crude residue was purified by Prep-TLC (CH₂Cl₂/MeOH=20:1) to give 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-4-{2-[(2S)-2-(2-isopropoxyphenyl)-4-(oxan-4-ylmethyl)piperazin-1-yl]-7-azaspiro[3.5]nonan-7-yl}-N-[3-nitro-4-({[(1r,4r)-4-hydroxy-4-methylcyclohexyl]methyl}amino)benzenesulfonyl]benzamide (assumed) as a yellow solid (10 mg, 20.9%). LC-MS (ESI, m/z) M+1: 1116. ¹H NMR (300 MHz, Chloroform-d) δ 12.58 (s, 1H), 8.64 (s, 1H), 8.46 (s, 1H), 8.39 (s, 1H), 8.11 (d, J=9.4 Hz, 2H), 7.81 (d, J=9.1 Hz, 1H), 7.05 (s, 2H), 6.95-6.78 (m, 3H), 6.70 (d, J=9.4 Hz, 1H), 6.63 (d, J=6.5 Hz, 1H), 6.42 (s, 1H), 6.11 (s, 1H), 4.77-4.42 (m, 4H), 3.96-3.94 (m, 3H), 3.66-3.16 (m, 9H), 3.05 (s, 5H), 2.77 (s, 3H), 2.35 (s, 2H), 1.86 (d, J=12.4 Hz, 2H), 1.58-1.55 (m, 13H), 1.40-1.26 (m, 10H), 1.25 (s, 2H), 1.20 (s, 5H).

### Example 160 Preparation of N-((4-((((1r,4r)-4-hydroxy-4-methylcyclohexyl)methyl)amino)-3-nitrophenyl)sulfonyl)-4-(2-((S)-2-(2-isopropoxyphenyl)-4-((1S,4R)-4-((R)-3-methylmorpholino)cyclohexyl)piperazin-1-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide (assumed)

Synthesis of (R)-3-methyl-4-(1,4-dioxaspiro[4.5]decan-8-yl)morpholine: Into a 100 mL round-bottom flask were added (3R)-3-methylmorpholine (1.9 g, 19.1 mmol, 1.0 eq), 1,4-dioxaspiro[4.5]decan-8-one (3.0 g, 19.1 mmol, 1.0 eq), NaBH₃CN (6.0 g, 95.9 mmol, 5.0 eq), ZnCl₂ (13.1 g, 95.9 mmol, 5.0 eq) and MeOH (30 mL, 10.0 eq). The reaction mixture was stirred for 2 hours at 50° C. The crude residue was purified by silica gel column chromatography, eluted with ethyl acetate/petroleum ether (1:5) to give (R)-3-methyl-4-(1,4-dioxaspiro[4.5]decan-8-yl)morpholineas a white oil (3.1 g, 66.9%). LC-MS (ESI, m/z) M+1: 242.

Synthesis of (R)-4-(3-methylmorpholino)cyclohexan-1-one: Into a 100 mL round-bottom flask were added (R)-3-methyl-4-(1,4-dioxaspiro[4.5]decan-8-yl)morpholine (1.0 g, 4.1 mmol, 1.0 eq), HCl (5 mL, 10.0 eq) and MeCN (5 mL, 10.0 eq). The resulting mixture was stirred for additional 6 hours at 25° C. The residue was basified to pH=6 with saturated NaHCO₃, and then extracted with CH₂Cl₂ (3×20 mL). The combined organic layers were washed with brine (10 mL), dried over anhydrous Na₂SO₄. After filtration, the filtrate was concentrated under vacuum. Finally, (R)-4-(3-methylmorpholino)cyclohexan-1-one was obtained as a white oil (300 mg, 36%). LC-MS (ESI, m/z) M+1: 198.

Synthesis of (R)-4-((1S,4S)-4-((R)-3-(2-isopropoxyphenyl)piperazin-1-yl)cyclohexyl)-3-methylmorpholine (assumed) and (R)-4-((1R,4R)-4-((R)-3-(2-isopropoxyphenyl)piperazin-1-yl)cyclohexyl)-3-methylmorpholine (assumed): Into a 40 mL vial were added (R)-4-(3-methylmorpholino)cyclohexan-1-one (300 mg, 1.5 mmol, 1.0 eq), (2R)-2-(2-isopropoxyphenyl)piperazine (assumed) (335 mg, 1.5 mmol, 1.0 eq), NaBH₃CN (477 mg, 7.6 mmol, 5.0 eq), ZnCl₂ (1036 mg, 7.6 mmol, 5.0 eq) and MeOH (4 mL, 10.0 eq). The resulting mixture was stirred for additional 2 hours at 50° C. The reaction was quenched by the addition of water (10 mL), and extracted with CH₂Cl₂ (3×5 mL). The combined organic layers were washed with brine (10 mL), dried over anhydrous Na₂SO₄. After filtration, the filtrate was concentrated under vacuum. The crude product was purified by Prep-HPLC using the following conditions to give (R)-4-((1S,4S)-4-((R)-3-(2-isopropoxyphenyl)piperazin-1-yl)cyclohexyl)-3-methylmorpholine (assumed) as a white oil (100 mg, 16.3%) and (R)-4-((1R,4R)-4-((R)-3-(2-isopropoxyphenyl)piperazin-1-yl)cyclohexyl)-3-methylmorpholine (assumed) a white oil (90 mg, 16%). LC-MS (ESI, m/z) M+1: 402.

Synthesis of N-((4-((((1r,4r)-4-hydroxy-4-methylcyclohexyl)methyl)amino)-3-nitrophenyl)sulfonyl)-4-(2-((S)-2-(2-isopropoxyphenyl)-4-((1S,4R)-4-((R)-3-methylmorpholino)cyclohexyl)piperazin-1-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide (assumed): Into an 8 mL vial were added (3R)-3-methyl-4-[(1s,4s)-4-[(3R)-3-(2-isopropoxyphenyl)piperazin-1-yl]cyclohexyl]morpholine (assumed) (19 mg, 0.1 mmol, 1.0 eq), 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-N-{3-nitro-4-[(oxan-4-ylmethyl)amino]benzenesulfonyl}-4-{2-oxo-7-azaspiro[3.5]nonan-7-yl}benzamide (38 mg, 0.1 mmol, 1.0 eq), NaBH₃CN (15 mg, 0.2 mmol, 5.0 eq), ZnCl₂ (33 mg, 0.2 mmol, 5.0 eq) and MeOH (1 mL, 0.5 mmol, 10.0 eq). The resulting mixture was stirred for additional 4 h at 70° C. The resulting mixture was diluted with CH₂Cl₂ (3×5 mL), and washed with brine (3×10 mL). The organic layer was dried over anhydrous Na₂SO₄. After filtration, the filtrate was concentrated under vacuum. The crude residue was purified by Prep-TLC (CH₂Cl₂/MeOH=18:1) to give N-((4-((((1r,4r)-4-hydroxy-4-methylcyclohexyl)methyl)amino)-3-nitrophenyl)sulfonyl)-4-(2-((S)-2-(2-isopropoxyphenyl)-4-((1S,4R)-4-((R)-3-methylmorpholino)cyclohexyl)piperazin-1-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide (assumed) as a yellow solid (10 mg, 17.0%). LC-MS (ESI, m/z) M+1: 1200. ¹H NMR (400 MHz, Chloroform-d) δ 12.59 (s, 1H), 8.65 (d, J=2.3 Hz, 1H), 8.46 (t, J=5.4 Hz, 1H), 8.39 (s, 1H), 8.15-8.07 (m, 1H), 7.81 (d, J=9.1 Hz, 1H), 7.43 (s, 1H), 7.17 (s, 1H), 7.10 (t, J=2.9 Hz, 1H), 6.90-6.81 (m, 3H), 6.71 (d, J=9.3 Hz, 1H), 6.64 (s, 1H), 6.43 (s, 1H), 6.11 (s, 1H), 4.45 (dd, J=10.9, 5.0 Hz, 1H), 3.99 (d, J=11.6 Hz, 1H), 3.87 (s, 1H), 3.80 (d, J=10.9 Hz, 1H), 3.67 (d, J=10.8 Hz, 1H), 3.60 (s, 1H), 3.51 (d, J=10.5 Hz, 2H), 3.48-3.39 (m, 1H), 3.29-3.17 (m, 4H), 3.11 (s, 2H), 3.06 (s, 2H), 2.99 (d, J=12.7 Hz, 3H), 2.73 (s, 3H), 2.65 (s, 1H), 2.49 (s, 1H), 2.32 (s, 1H), 2.22 (s, 1H), 2.03 (s, 2H), 1.87 (d, J=12.8 Hz, 3H), 1.77-1.75 (m, 4H), 1.60 (s, 12H), 1.53 (d, J=3.7 Hz, 1H), 1.43 (s, 3H), 1.34 (s, 10H), 1.26-1.16 (m, 4H).

### Example 161 Preparation of N-((4-((((1r,4r)-4-hydroxy-4-methylcyclohexyl)methyl)amino)-3-nitrophenyl)sulfonyl)-4-(2-((S)-2-(2-isopropoxyphenyl)-4-((1R,4S)-4-((R)-3-methylmorpholino)cyclohexyl)piperazin-1-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide (assumed)

Synthesis of N-((4-((((1r,4r)-4-hydroxy-4-methylcyclohexyl)methyl)amino)-3-nitrophenyl)sulfonyl)-4-(2-((S)-2-(2-isopropoxyphenyl)-4-((1R,4S)-4-((R)-3-methylmorpholino)cyclohexyl)piperazin-1-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide (assumed): Into an 8 mL vial were added (R)-4-((1R,4R)-4-((R)-3-(2-isopropoxyphenyl)piperazin-1-yl)cyclohexyl)-3-methylmorpholine (assumed) (19 mg, 0.1 mmol, 1.0 eq), 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-N-{3-nitro-4-[(oxan-4-ylmethyl)amino]benzenesulfonyl}-4-{2-oxo-7-azaspiro[3.5]nonan-7-yl}benzamide (43 mg, 0.1 mmol, 1.0 eq), NaBH₃CN (17 mg, 0.3 mmol, 5.0 eq), ZnCl₂ (37 mg, 0.3 mmol, 5.0 eq) and MeOH (1 mL, 0.5 mmol, 10.0 eq). The resulting mixture was stirred for additional 4 hours at 70° C. The resulting mixture was diluted with CH₂Cl₂ (3×5 mL) and washed with brine (3×10 mL). The organic layer was dried over anhydrous Na₂SO₄. After filtration, the filtrate was concentrated under vacuum. The crude residue was purified by Prep-TLC (CH₂Cl₂/MeOH=18:1) to give N-((4-((((1r,4r)-4-hydroxy-4-methylcyclohexyl)methyl)amino)-3-nitrophenyl)sulfonyl)-4-(2-((S)-2-(2-isopropoxyphenyl)-4-((1R,4S)-4-((R)-3-methylmorpholino)cyclohexyl)piperazin-1-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide (assumed) as a yellow solid (10 mg, 15.1%). LC-MS (ESI, m/z) M+1: 1200. ¹H NMR (400 MHz, Chloroform-d) δ 12.59 (s, 1H), 8.65 (s, 1H), 8.47 (t, J=5.3 Hz, 1H), 8.36 (s, 1H), 8.11 (d, J=9.2 Hz, 1H), 7.81 (d, J=9.9 Hz, 1H), 7.48 (d, J=7.5 Hz, 1H), 7.17 (s, 1H), 7.10 (t, J=3.0 Hz, 1H), 6.86 (s, 2H), 6.71 (d, J=9.2 Hz, 1H), 6.65 (s, 1H), 6.44 (s, 1H), 6.12 (s, 1H), 4.55 (d, J=5.5 Hz, 1H), 4.46 (dd, J=10.9, 5.0 Hz, 1H), 3.99 (d, J=11.1 Hz, 1H), 3.86 (d, J=9.9 Hz, 1H), 3.71 (s, 3H), 3.47 (dt, J=23.7, 10.9 Hz, 4H), 3.21 (t, J=6.1 Hz, 2H), 3.13 (s, 2H), 3.06 (s, 3H), 2.98 (s, 2H), 2.82 (d, J=15.8 Hz, 1H), 2.64 (s, 2H), 2.56 (s, 1H), 2.24 (s, 2H), 2.16 (s, 1H), 2.07 (s, 1H), 1.87 (d, J=13.0 Hz, 5H), 1.77-1.75 (m, 5H), 1.59-1.56 (m, 16H), 1.44 (s, 2H), 1.38-1.26 (m, 11H), 1.23 (d, J=11.6 Hz, 2H), 1.00 (d, J=6.3 Hz, 2H).

### Example 162 Preparation of N-((4-((((1r,4r)-4-hydroxy-4-methylcyclohexyl)methyl)amino)-3-nitrophenyl)sulfonyl)-4-(2-((S)-2-(2-isopropoxyphenyl)-4-(1-(tetrahydro-2H-pyran-4-yl)piperidin-4-yl)piperazin-1-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide (assumed)

Synthesis of 8-(oxan-4-yl)-1,4-dioxa-8-azaspiro[4.5]decane: Into a 100 mL round-bottom flask were added 1,4-dioxa-8-azaspiro[4.5]decane (2.8 g, 19.9 mmol, 1.0 eq), 4H-pyran-4-one (2.0 g, 19.9 mmol, 1.0 eq), NaBH₃CN (6.2 g, 99.8 mmol, 5 eq), ZnCl₂ (13.6 g, 99.8 mmol, 5.0 eq) and MeOH (20 mL, 199.7 mmol, 10.0 eq). The resulting mixture was stirred for additional overnight at 50° C. The crude residue was purified by silica gel column chromatography, eluted with ethyl acetate/petroleum ether (1:2) to give 8-(oxan-4-yl)-1,4-dioxa-8-azaspiro[4.5]decane as a white oil (2.6 g, 57.2%). LC-MS (ESI, m/z) M+1: 228.

Synthesis of 1-(oxan-4-yl)piperidin-4-one: Into a 100 mL round-bottom flask were added 8-(oxan-4-yl)-1,4-dioxa-8-azaspiro[4.5]decane (1.0 g, 4.4 mmol, 1.0 eq) and HCl (6 M) (10.0 mL, 43.9 mmol, 10.0 eq). The resulting mixture was stirred for additional overnight at 50° C. The residue was basified to pH=6 with saturated NaHCO₃ and then extracted with CH₂Cl₂ (3×10 mL). The combined organic layers were washed with brine (3×10 mL), dried over anhydrous Na₂SO₄. After filtration, the filtrate was concentrated under vacuum. Finally, 1-(oxan-4-yl)piperidin-4-one was obtained as a white solid (310 mg, 38.4%). LC-MS (ESI, m/z) M+1: 184.

Synthesis of (3S)-3-(2-isopropoxyphenyl)-1-[1-(oxan-4-yl)piperidin-4-yl]piperazine (assumed): Into a 40 mL vial were added 1-(oxan-4-yl)piperidin-4-one (300 mg, 1.6 mmol, 1.0 eq), (2S)-2-(2-isopropoxyphenyl)piperazine (assumed) (360 mg, 1.6 mmol, 1.0 eq), NaBH₃CN (514 mg, 8.2 mmol, 5.0 eq), ZnCl₂ (1115 mg, 8.2 mmol, 5.0 eq) and MeOH (4 mL, 16.4 mmol, 10.0 eq) The resulting mixture was stirred for additional 3 hours at 25° C. The crude residue was purified by Prep-TLC (CH₂Cl₂/MeOH=15:1) to give (3S)-3-(2-isopropoxyphenyl)-1-[1-(oxan-4-yl)piperidin-4-yl]piperazines a white oil (assumed) (163 mg, 25.7%). LC-MS (ESI, m/z) M+1: 388.

Synthesis of N-((4-((((1r,4r)-4-hydroxy-4-methylcyclohexyl)methyl)amino)-3-nitrophenyl)sulfonyl)-4-(2-((S)-2-(2-isopropoxyphenyl)-4-(1-(tetrahydro-2H-pyran-4-yl)piperidin-4-yl)piperazin-1-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide (assumed): Into an 8 mL vial were added (3S)-3-(2-isopropoxyphenyl)-1-[1-(oxan-4-yl)piperidin-4-yl]piperazine (assumed) (38 mg, 0.1 mmol, 2.0 eq), 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-N-[3-nitro-4-({[(1r,4r)-4-hydroxy-4-methylcyclohexyl]methyl}amino)benzenesulfonyl]-4-{2-oxo-7-azaspiro[3.5]nonan-7-yl}benzamide (39 mg, 0.1 mmol, 1.0 eq), NaBH₃CN (15 mg, 0.2 mmol, 5.0 eq), ZnCl₂ (33 mg, 0.2 mmol, 5.0 eq) and MeOH (1 mL, 10.0 eq). The resulting mixture was stirred for 4 hours at 70° C. The resulting mixture was diluted with CH₂Cl₂ (3×5 mL). The combined organic layers were washed with brine (10 mL), dried over anhydrous Na₂SO₄. After filtration, the filtrate was concentrated under vacuum. The crude residue was purified by Prep-TLC (CH₂Cl₂/MeOH=18:1) to give N-((4-((((1r,4r)-4-hydroxy-4-methylcyclohexyl)methyl)amino)-3-nitrophenyl)sulfonyl)-4-(2-((S)-2-(2-isopropoxyphenyl)-4-(1-(tetrahydro-2H-pyran-4-yl)piperidin-4-yl)piperazin-1-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide (assumed) as a yellow solid (10 mg, 17%). LC-MS (ESI, m/z) M+1: 1186. ¹H NMR (300 MHz, Chloroform-d) δ 12.59 (s, 1H), 8.65 (d, J=2.2 Hz, 1H), 8.47 (s, 1H), 8.39 (s, 1H), 8.11 (d, J=9.1 Hz, 1H), 7.81 (d, J=8.2 Hz, 1H), 7.41 (s, 1H), 7.17 (s, 1H), 7.14-7.06 (m, 1H), 6.84 (d, J=9.3 Hz, 2H), 6.71 (d, J=9.3 Hz, 1H), 6.64 (s, 1H), 6.43 (s, 1H), 6.11 (s, 1H), 4.77-4.36 (m, 4H), 4.02 (s, 4H), 3.44-3.42 (m, 5H), 3.23-2.86 (m, 8H), 2.25 (s, 9H), 1.95-1.64 (m, 11H), 1.55-1.42 (m, 5H), 1.34-1.30 (m, 5H), 1.28-1.26 (m, 10H), 0.96-0.78 (m, 5H).

### Example 163 Preparation of N-((4-((((1r,4r)-4-hydroxy-4-methylcyclohexyl)methyl)amino)-3-nitrophenyl)sulfonyl)-4-(2-((S)-2-(2-isopropoxyphenyl)-4-((6-methoxy-5-morpholinopyridin-3-yl)methyl)piperazin-1-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide (assumed)

Synthesis of 4-(5-bromo-2-methoxypyridin-3-yl)morpholine: Into a 250 mL round-bottom flask were added 5-bromo-2-methoxypyridin-3-amine (5.0 g, 24.6 mmol, 1.0 eq), 1-bromo-2-(2-bromoethoxy)ethane (6.8 g, 29.5 mmol, 1.2 eq), DIEA (9.5 g, 73.8 mmol, 3.0 eq) and DMF (50 mL). The resulting mixture was stirred for additional 24 hours at 120° C. The resulting mixture was filtered, the filter cake was washed with EtOAc (3×20 mL). The filtrate was concentrated under reduced pressure. The resulting mixture was extracted with EtOAc (3×40 mL). The combined organic layers were washed with brine (3×20 mL), dried over anhydrous Na₂SO₄. After filtration, the filtrate was concentrated under vacuum. The crude residue was purified by reversed-phase flash chromatography using the following conditions: column, C18 silica gel; mobile phase, MeCN in water (0.1% NH₃·H₂O), 10% to 70% gradient in 10 min; detector, UV 254 nm. Finally, 4-(5-bromo-2-methoxypyridin-3-yl)morpholine was obtained as a light yellow solid (1.7 g, 25.8%). LC-MS (ESI, m/z) M+1: 273/275.

Synthesis of 6-methoxy-5-(morpholin-4-yl)pyridine-3-carbaldehyde: A solution of 4-(5-bromo-2-methoxypyridin-3-yl)morpholine (500 mg, 1.8 mmol, 1.0 eq) in THE (7 mL) was treated with n-BuLi (0.8 mL, 9.3 mmol, 1.2 eq) for 30 min at −78° C. under nitrogen atmosphere. This was followed by the addition of DMF (805 mg, 9.3 mmol, 6.0 eq) dropwise at −78° C. The resulting mixture was stirred for additional 1 hours at −78° C. The reaction was quenched by the addition of sat. NH₄Cl (8 mL) at 0° C., and then extracted with EtOAc (3×10 mL). The combined organic layers were washed with brine (5 mL), dried over anhydrous Na₂SO₄. After filtration, the filtrate was concentrated under vacuum. The crude residue was purified by silica gel column chromatography, eluted with ethyl acetate/petroleum ether=1:5 to give 6-methoxy-5-(morpholin-4-yl)pyridine-3-carbaldehyde as a light yellow solid (120 mg, 29.5%). LC-MS (ESI, m/z) M+1: 223.

Synthesis of 4-(5-{[(3S)-3-(2-isopropoxyphenyl)piperazin-1-yl]methyl}-2-methoxypyridin-3-yl)morpholine (assumed): Into an 8 mL vial were added 6-methoxy-5-(morpholin-4-yl)pyridine-3-carbaldehyde (100 mg, 0.4 mmol, 1.0 eq), (2S)-2-(2-isopropoxyphenyl)piperazine (assumed) (99 mg, 0.4 mmol, 1.0 eq), NaBH(OAc)₃ (476 mg, 2.2 mmol, 5.0 eq) and DCE (1 mL, 4.5 mmol, 10.0 eq). The resulting mixture was stirred for additional 3 hours at 25° C. The reaction was quenched with water (1 mL) and extracted with CH₂Cl₂ (3×5 mL). The combined organic layers were washed with brine (5 mL), dried over anhydrous Na₂SO₄. After filtration, the filtrate was concentrated under vacuum. The crude residue was purified by Prep-TLC (CH₂Cl₂/MeOH=10:1) to give 4-(5-{[(3S)-3-(2-isopropoxyphenyl)piperazin-1-yl]methyl}-2-methoxypyridin-3-yl)morpholine (assumed) as a yellow oil (109 mg, 56.7%). LC-MS (ESI, m/z) M+1: 427.

Synthesis of N-((4-((((1r,4r)-4-hydroxy-4-methylcyclohexyl)methyl)amino)-3-nitrophenyl)sulfonyl)-4-(2-((S)-2-(2-isopropoxyphenyl)-4-((6-methoxy-5-morpholinopyridin-3-yl)methyl)piperazin-1-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide (assumed): Into an 8 mL vial were added 4-(5-{[(3S)-3-(2-isopropoxyphenyl)piperazin-1-yl]methyl}-2-methoxypyridin-3-yl)morpholine (assumed) (21 mg, 0.1 mmol, 1.0 eq), 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-N-[3-nitro-4-({[(1r,4r)-4-hydroxy-4-methylcyclohexyl]methyl}amino)benzenesulfonyl]-4-{2-oxo-7-azaspiro[3.5]nonan-7-yl}benzamide (40 mg, 0.1 mmol, 1.0 eq), NaBH₃CN (15 mg, 0.3 mmol, 5.0 eq), ZnCl₂ (33 mg, 0.3 mmol, 5.0 eq), and MeOH (1 mL, 0.4 mmol, 10.0 eq). The resulting mixture was stirred for additional 4 hours at 70° C. The reaction was quenched with water (5 mL), and extracted with CH₂Cl₂ (3×5 mL). The combined organic layers were washed with brine (10 mL), dried over anhydrous Na₂SO₄. After filtration, the filtrate was concentrated under vacuum. The crude residue was purified by Prep-TLC (CH₂Cl₂/MeOH=18:1) to give N-((4-((((1r,4r)-4-hydroxy-4-methylcyclohexyl)methyl)amino)-3-nitrophenyl)sulfonyl)-4-(2-((S)-2-(2-isopropoxyphenyl)-4-((6-methoxy-5-morpholinopyridin-3-yl)methyl)piperazin-1-yl)-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide (assumed) as a yellow solid (10 mg, 16.5%). LC-MS (ESI, m/z) M+1: 1225. ¹H NMR (300 MHz, Chloroform-d) δ 12.59 (s, 1H), 8.65 (d, J=2.3 Hz, 1H), 8.41 (d, J=30.7 Hz, 2H), 8.10 (d, J=9.2 Hz, 1H), 7.81 (d, J=8.5 Hz, 1H), 7.69-7.66 (m, 1H), 7.43 (d, J=7.5 Hz, 1H), 7.16 (d, J=7.7 Hz, 1H), 7.07-7.02 (m, 2H), 6.83-6.79 (m, 2H), 6.74-6.66 (m, 1H), 6.63 (d, J=6.7 Hz, 1H), 6.43 (s, 1H), 6.11 (s, 1H), 4.65-4.34 (m, 4H), 3.98 (s, 3H), 3.89 (s, 3H), 3.48-3.44 (m, 5H), 3.21 (t, J=6.2 Hz, 3H), 3.08-3.01 (m, 6H), 2.90-2.86 (m, 3H), 2.62-2.58 (m, 3H), 2.36-2.07 (m, 4H), 1.86-1.84 (m, 3H), 1.77-1.72 (m, 4H), 1.58-1.53 (m, 12H), 1.45-1.42 (m, 3H), 1.34-1.32 (m, 3H), 1.21 (s, 2H).

### Example 164 Preparation of 4-{2-[(2R)-4-[(3,4-difluorophenyl)methyl]-2-(2-methylphenyl)piperazin-1-yl]-7-azaspiro[3.5]nonan-7-yl}-2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-N-{3-nitro-4-[(oxan-4-ylmethyl)amino]benzenesulfonyl}benzamide

Synthesis of (3R)-1-[(3,4-difluorophenyl)methyl]-3-(2-methylphenyl)piperazine: Into an 8-mL sealed tube, were placed 3,4-difluorobenzaldehyde (200 mg, 1.4 mmol, 1.0 eq), (2R)-2-(2-methylphenyl)piperazine (248 mg, 1.4 mmol, 1.0 eq), NaBH₃CN (265 mg, 4.2 mmol, 3.0 eq), ZnCl₂ (575 mg, 4.2 mmol, 3.0 eq), MeOH (2 mL). The resulting solution was stirred for 2 hours at 25° C. The resulting mixture was then quenched by the addition of water (30 mL), and extracted with dichloromethane (2×30 mL). The combined organic layers was washed with brine (2×30 mL), dried over anhydrous Na₂SO₄. After filtration, the filtrate was concentrated under vacuum. The crude residue was applied onto a silica gel column and eluted with dichloromethane/methanol=10:1 to give (3R)-1-[(3,4-difluorophenyl)methyl]-3-(2-methylphenyl)piperazine as a white solid (260 mg, 61.1%). LC-MS (ESI, m/z) M+1: 303.

Synthesis of methyl 4-{2-[(2R)-4-[(3,4-difluorophenyl)methyl]-2-(2-methylphenyl)piperazin-1-yl]-7-azaspiro[3.5]nonan-7-yl}-2-[(3R,8S)-15-{[2-(trimethylsilyl)ethoxy]methyl}-2,5-dioxa-9,15,17-triazatetracyclo[8.7.0.0\hat{ }{3,8}.0\hat{ }{12,16}]heptadeca-1(10),11,13,16-tetraen-9-yl]benzoate: Into an 8-mL sealed tube, were placed methyl 4-{2-oxo-7-azaspiro[3.5]nonan-7-yl}-2-[(3R,8S)-15-{[2-(trimethylsilyl)ethoxy]methyl}-2,5-dioxa-9,15,17-triazatetracyclo[8.7.0.0\hat{ }{3,8}.0\hat{ }{12,16}]heptadeca-1(10),11,13,16-tetraen-9-yl]benzoate (300 mg, 0.5 mmol, 1.0 eq), (3R)-1-[(3,4-difluorophenyl)methyl]-3-(2-methylphenyl)piperazine (215 mg, 0.7 mmol, 1.5 eq), ZnCl₂ (194 mg, 1.4 mmol, 3.0 eq), NaBH₃CN (89 mg, 1.4 mmol, 3.0 eq), MeOH:DCE (1:1, 3 mL). The resulting solution was stirred for overnight at 50° C. The resulting mixture was then quenched by the addition of water (30 mL), and extracted with dichloromethane (2×30 mL). The combined organic layers was washed with brine (2×30 mL), dried over anhydrous Na₂SO₄. After filtration, the filtrate was concentrated under vacuum. The crude residue was applied onto a silica gel column and eluted with dichloromethane/methanol=10:1 to give methyl 4-{2-[(2R)-4-[(3,4-difluorophenyl)methyl]-2-(2-methylphenyl)piperazin-1-yl]-7-azaspiro[3.5]nonan-7-yl}-2-[(3R,8S)-15-{[2-(trimethylsilyl)ethoxy]methyl}-2,5-dioxa-9,15,17-triazatetracyclo[8.7.0.0\hat{ }{3,8}.0\hat{ }{12,16}]heptadeca-1(10),11,13,16-tetraen-9-yl]benzoate as a light yellow solid (300 mg, 68.9%). LC-MS (ESI, m/z) M+1: 919.

Synthesis of 4-{2-[(2R)-4-[(3,4-difluorophenyl)methyl]-2-(2-methylphenyl)piperazin-1-yl]-7-azaspiro[3.5]nonan-7-yl}-2-[(3R,8S)-15-{[2-(trimethylsilyl)ethoxy]methyl}-2,5-dioxa-9,15,17-triazatetracyclo[8.7.0.0\hat{ }{3,8}.0\hat{ }{12,16}]heptadeca-1(10),11,13,16-tetraen-9-yl]benzoic acid: Into an 8 mL seal tube, were placed methyl 4-{2-[(2R)-4-[(3,4-difluorophenyl)methyl]-2-(2-methylphenyl)piperazin-1-yl]-7-azaspiro[3.5]nonan-7-yl}-2-[(3R,8S)-15-{[2-(trimethylsilyl)ethoxy]methyl}-2,5-dioxa-9,15,17-triazatetracyclo[8.7.0.0\hat{ }{3,8}.0\hat{ }{12,16}]heptadeca-1(10),11,13,16-tetraen-9-yl]benzoate (300 mg, 0.3 mmol, 1.0 eq), 1,4-dioxane:MeOH=1:1 (3 mL), NaOH (52 mg, 1.3 mmol, 4.0 eq). The reaction mixture was stirred for overnight at 50° C. The resulting mixture was then diluted with water (30 mL) and acidified to pH=5 with HCl (1 M). The precipitated solids were collected by filtration and dried under infrared light to give 4-{2-[(2R)-4-[(3,4-difluorophenyl)methyl]-2-(2-methylphenyl)piperazin-1-yl]-7-azaspiro[3.5]nonan-7-yl}-2-[(3R,8S)-15-{[2-(trimethylsilyl)ethoxy]methyl}-2,5-dioxa-9,15,17-triazatetracyclo[8.7.0.0\hat{ }{3,8}.0\hat{ }{12,16}]heptadeca-1(10),11,13,16-tetraen-9-yl]benzoic acid as a light yellow solid (260 mg, 88.0%). LC-MS (ESI, m/z) M+1: 905.

Synthesis of 4-{2-[(2R)-4-[(3,4-difluorophenyl)methyl]-2-(2-methylphenyl)piperazin-1-yl]-7-azaspiro[3.5]nonan-7-yl}-N-{3-nitro-4-[(oxan-4-ylmethyl)amino]benzenesulfonyl}-2-[(3R,8S)-15-{[2-(trimethylsilyl)ethoxy]methyl}-2,5-dioxa-9,15,17-triazatetracyclo[8.7.0.0\hat{ }{3,8}.0\hat{ }{12,16}]heptadeca-1(10),11,13,16-tetraen-9-yl]benzamide: Into an 8 mL vial, were placed 4-{2-[(2R)-4-[(3,4-difluorophenyl)methyl]-2-(2-methylphenyl)piperazin-1-yl]-7-azaspiro[3.5]nonan-7-yl}-2-[(3R,8S)-15-{[2-(trimethylsilyl)ethoxy]methyl}-2,5-dioxa-9,15,17-triazatetracyclo[8.7.0.0\hat{ }{3,8}.0\hat{ }{12,16}]heptadeca-1(10),11,13,16-tetraen-9-yl]benzoic acid (80 mg, 0.1 mmol, 1.0 eq), 3-nitro-4-[(oxan-4-ylmethyl)amino]benzenesulfonamide (22 mg, 0.1 mmol, 0.8 eq), dichloromethane (1 mL), EDCl (34 mg, 0.2 mmol, 2.0 eq), DMAP (11 mg, 0.1 mmol, 1.0 eq). The reaction mixture was stirred for 4 hours at 25° C. The resulting mixture was then quenched by the addition of water (50 mL), and extracted with dichloromethane (2×50 mL). The combined organic layers was washed with brine (2×50 mL), dried over anhydrous Na₂SO₄. The resulting mixture was concentrated under vacuum. The crude residue was applied onto a silica gel column and eluted with dichloromethane/methanol=20:1 to give 4-{2-[(2R)-4-[(3,4-difluorophenyl)methyl]-2-(2-methylphenyl)piperazin-1-yl]-7-azaspiro[3.5]nonan-7-yl}-N-{3-nitro-4-[(oxan-4-ylmethyl)amino]benzenesulfonyl}-2-[(3R,8S)-15-{[2-(trimethylsilyl)ethoxy]methyl}-2,5-dioxa-9,15,17-triazatetracyclo[8.7.0.0\hat{ }{3,8}.0\hat{ }{12,16}]heptadeca-1(10),11,13,16-tetraen-9-yl]benzamide as a yellow solid (40 mg, 37.6%). LC-MS (ESI, m/z) M+1: 1202.

Synthesis of 4-{2-[(2R)-4-[(3,4-difluorophenyl)methyl]-2-(2-methylphenyl)piperazin-1-yl]-7-azaspiro[3.5]nonan-7-yl}-2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-N-{3-nitro-4-[(oxan-4-ylmethyl)amino]benzenesulfonyl}benzamide: Into an 8-mL sealed tube, were placed 4-{2-[(2R)-4-[(3,4-difluorophenyl)methyl]-2-(2-methylphenyl)piperazin-1-yl]-7-azaspiro[3.5]nonan-7-yl}-N-{3-nitro-4-[(oxan-4-ylmethyl)amino]benzenesulfonyl}-2-[(3R,8S)-15-{[2-(trimethylsilyl)ethoxy]methyl}-2,5-dioxa-9,15,17-triazatetracyclo[8.7.0.0\hat{ }{3,8}.0\hat{ }{12,16}]heptadeca-1(10),11,13,16-tetraen-9-yl]benzamide (40 mg, 0.03 mmol, 1.0 eq), ethylenediamine (40 mg, 0.6 mmol, 20.0 eq), TBAF in THE (1 M, 1 mL). The resulting solution was stirred for 3 hours at 80° C. The resulting mixture was then quenched by the addition of water (200 mL), and extracted with ethyl acetate (2×100 mL). The combined organic layers was washed with brine (2×100 mL) and dried over anhydrous Na₂SO₄. After filtration, the filtrate was concentrated under vacuum. The crude residue was applied onto a silica gel column and eluted with dichloromethane/methanol=20:1 to give 4-{2-[(2R)-4-[(3,4-difluorophenyl)methyl]-2-(2-methylphenyl)piperazin-1-yl]-7-azaspiro[3.5]nonan-7-yl}-2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-N-{3-nitro-4-[(oxan-4-ylmethyl)amino]benzenesulfonyl}benzamide as a yellow solid (10 mg, 28.0%). LC-MS (ESI, m/z) M+1: 1072. ¹H NMR (400 MHz, Methanol-d₄) δ 8.58-8.41 (m, 1H), 7.97-7.84 (m, 1H), 7.64-7.38 (m, 2H), 7.36-7.09 (m, 6H), 7.08-7.06 (m, 1H), 6.99-6.87 (m, 1H), 6.78-6.60 (m, 2H), 6.53-6.44 (m, 1H), 6.05-5.91 (m, 1H), 4.37-4.26 (m, 2H), 4.03-3.88 (m, 3H), 3.78-3.65 (m, 2H), 3.62-3.41 (m, 5H), 3.31-3.25 (m, 2H), 3.20-3.05 (m, 5H), 2.97-2.91 (m, 1H), 2.70-2.61 (m, 1H), 2.41-2.23 (m, 5H), 1.78-1.70 (m, 3H), 1.49-1.29 (m, 12H), 1.18-1.13 (m, 1H), 0.97-0.88 (m, 2H).

### Example 165 Preparation of 4-{2-[(2R)-4-[(3,4-difluorophenyl)methyl]-2-(2-isopropylphenyl)piperazin-1-yl]-7-azaspiro[3.5]nonan-7-yl}-2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-N-{3-nitro-4-[(oxan-4-ylmethyl)amino]benzenesulfonyl}benzamide (assumed)

Synthesis of N-((3-nitro-4-(((tetrahydro-2H-pyran-4-yl)methyl)amino)phenyl)sulfonyl)-4-(2-oxo-7-azaspiro[3.5]nonan-7-yl)-2-((5aS,9aR)-1-((2-(trimethylsilyl)ethoxy)methyl)-6,7,9,9a-tetrahydro-1H-pyrano[3,4-b]pyrrolo[3′,2′:5,6]pyrido[3,2-e][1,4]oxazin-5(5aH)-yl)benzamide: Into a 40 mL vial, were placed (3R,8S)-15-{[2-(trimethylsilyl)ethoxy]methyl}-2,5-dioxa-9,15,17-triazatetracyclo[8.7.0.0\hat{ }{3,8}.0\hat{ }{12,16}]heptadeca-1(10),11,13,16-tetraene (210 mg, 0.6 mmol, 1.0 eq), 2-bromo-N-((3-nitro-4-(((tetrahydro-2H-pyran-4-yl)methyl)amino)phenyl)sulfonyl)-4-(2-oxo-7-azaspiro[3.5]nonan-7-yl)benzamide (500 mg, 0.8 mmol, 1.3 eq), DMSO (10 mL), K₂CO₃ (241 mg, 1.7 mmol, 3.0 eq), Cul (55 mg, 0.3 mmol, 0.5 eq), N1,N2-diphenyloxalamide (70 mg, 0.3 mmol, 0.5 eq). The reaction mixture was stirred for 3 hours at 100° C. under nitrogen atmosphere. The resulting mixture was quenched by the addition of water (200 mL) and extracted with EtOAc (3×100 mL). The combined organic layer was washed with brine (200 mL), dried over anhydrous Na₂SO₄. After filtration, the filtrate was concentrated under vacuum. The crude residue was applied onto a silica gel column and eluted with dichloromethane/methanol=10:1 to give N-((3-nitro-4-(((tetrahydro-2H-pyran-4-yl)methyl)amino)phenyl)sulfonyl)-4-(2-oxo-7-azaspiro[3.5]nonan-7-yl)-2-((5aS,9aR)-1-((2-(trimethylsilyl)ethoxy)methyl)-6,7,9,9a-tetrahydro-1H-pyrano[3,4-b]pyrrolo[3′,2′:5,6]pyrido[3,2-e][1,4]oxazin-5(5aH)-yl)benzamide as a yellow solid (220 mg, 40.1%). LC-MS (ESI, m/z) M+1: 916.

Synthesis of N-((3-nitro-4-(((tetrahydro-2H-pyran-4-yl)methyl)amino)phenyl)sulfonyl)-4-(2-oxo-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide: Into a 40 mL vial, were placed N-((3-nitro-4-(((tetrahydro-2H-pyran-4-yl)methyl)amino)phenyl)sulfonyl)-4-(2-oxo-7-azaspiro[3.5]nonan-7-yl)-2-((5aS,9aR)-1-((2-(trimethylsilyl)ethoxy)methyl)-6,7,9,9a-tetrahydro-1H-pyrano[3,4-b]pyrrolo[3′,2′:5,6]pyrido[3,2-e][1,4]oxazin-5(5aH)-yl)benzamide (1.5 g, 1.6 mmol, 1.0 eq), ethylenediamine (100 mg, 1.6 mmol, 1.0 eq), TBAF in THE (1 M, 20 mL). The resulting mixture was stirred for 4 hours at 70° C. under N₂ atmosphere. The resulting mixture was diluted with EtOAc (500 mL) and washed with water (200 mL). The organic layer was dried over anhydrous Na₂SO₄. After filtration, the filtrate was concentrated under vacuum. The crude residue was applied onto a silica gel column and eluted with dichloromethane/methanol=10:1 to give N-((3-nitro-4-(((tetrahydro-2H-pyran-4-yl)methyl)amino)phenyl)sulfonyl)-4-(2-oxo-7-azaspiro[3.5]nonan-7-yl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide as a yellow solid. (1.0 g, 77.3%). LC-MS (ESI, m/z) M+1: 786.

Synthesis of (3R)-1-[(3,4-difluorophenyl)methyl]-3-(2-isopropylphenyl)piperazine (assumed): Into an 8-mL sealed tube, were placed 3,4-difluorobenzaldehyde (56 mg, 0.4 mmol, 1.0 eq), (2R)-2-(2-isopropylphenyl)piperazine (assumed) (81 mg, 0.4 mmol, 1.0 eq), NaBH(OAc)₃ (251 mg, 1.2 mmol, 3.0 eq), DCE (0.5 mL). The resulting solution was stirred for 2 hours at 25° C. The resulting mixture was then quenched by the addition of water (30 mL), and extracted with dichloromethane (2×30 mL). The combined organic layer was washed with brine (2×30 mL), dried over anhydrous Na₂SO₄. After filtration, the filtrate was concentrated under vacuum. The crude residue was applied onto a silica gel column and eluted with dichloromethane/methanol=10:1 to give (3R)-1-[(3,4-difluorophenyl)methyl]-3-(2-isopropylphenyl)piperazine (assumed) as a light yellow oil (80 mg, 61.4%). LC-MS (ESI, m/z) M+1: 331.

Synthesis of 4-{2-[(2R)-4-[(3,4-difluorophenyl)methyl]-2-(2-isopropylphenyl)piperazin-1-yl]-7-azaspiro[3.5]nonan-7-yl}-2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-N-{3-nitro-4-[(oxan-4-ylmethyl)amino]benzenesulfonyl}benzamide (assumed): Into an 8-mL sealed tube, were placed 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-N-{3-nitro-4-[(oxan-4-ylmethyl)amino]benzenesulfonyl}-4-{2-oxo-7-azaspiro[3.5]nonan-7-yl}benzamide (70 mg, 0.1 mmol, 1.0 eq), (3R)-1-[(3,4-difluorophenyl)methyl]-3-(2-isopropylphenyl)piperazine (assumed) (29 mg, 0.1 mmol, 1.0 eq), ZnCl₂ (36 mg, 0.3 mmol, 3.0 eq), NaBH₃CN (17 mg, 0.3 mmol, 3.0 eq), MeOH (1 mL). The resulting solution was stirred for 4 hours at 70° C. The resulting mixture was then quenched by the addition of water (30 mL), and extracted with dichloromethane (2×30 mL). The combined organic layer was washed with brine (2×30 mL) and dried over anhydrous Na₂SO₄. After filtration, the filtrate was concentrated under vacuum. The crude residue was applied onto a silica gel column and eluted with dichloromethane/methanol=20:1 to give 4-{2-[(2R)-4-[(3,4-difluorophenyl)methyl]-2-(2-isopropylphenyl)piperazin-1-yl]-7-azaspiro[3.5]nonan-7-yl}-2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-N-{3-nitro-4-[(oxan-4-ylmethyl)amino]benzenesulfonyl}benzamide (assumed) as a yellow solid (10 mg, 10.2%). LC-MS (ESI, m/z) M+1: 1100. ¹H NMR (400 MHz, Methanol-d₄) δ 8.52-8.47 (m, 1H), 7.88-7.80 (m, 1H), 7.53-7.04 (m, 9H), 6.97-6.85 (m, 1H), 6.72-6.62 (m, 2H), 6.56-6.47 (m, 1H), 6.04-5.92 (m, 1H), 4.47-4.21 (m, 1H), 4.04-3.88 (m, 3H), 3.87-3.62 (m, 3H), 3.59-3.42 (m, 6H), 3.30-3.23 (m, 2H), 3.17-2.83 (m, 6H), 2.64-2.56 (m, 1H), 2.47-2.16 (m, 4H), 2.07-1.91 (m, 1H), 1.90-1.81 (m, 1H), 1.78-1.63 (m, 4H), 1.60-1.58 (m, 2H), 1.51-1.37 (m, 4H), 1.33-1.25 (m, 6H), 1.18-1.07 (m, 3H), 0.96-0.83 (m, 1H).

### Example 166 Preparation of 4-{2-[(2S)-4-[(3,4-difluorophenyl)methyl]-2-(2-isopropylphenyl)piperazin-1-yl]-7-azaspiro[3.5]nonan-7-yl}-2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-N-{3-nitro-4-[(oxan-4-ylmethyl)amino]benzenesulfonyl}benzamide (assumed)

Synthesis of (3S)-1-[(3,4-difluorophenyl)methyl]-3-(2-isopropylphenyl)piperazine (assumed): Into an 8-mL sealed tube, were placed 3,4-difluorobenzaldehyde (56 mg, 0.4 mmol, 1.0 eq), (2S)-2-(2-isopropylphenyl)piperazine (assumed) (81 mg, 0.4 mmol, 1.0 eq), NaBH(OAc)₃ (251 mg, 1.2 mmol, 3.0 eq), DCE (0.5 mL). The resulting solution was stirred for 2 hours at 25° C. The resulting mixture was then quenched by the addition of water (30 mL), and extracted with dichloromethane (2×30 mL). The combined organic layers was washed with brine (2×30 mL), dried over anhydrous Na₂SO₄. After filtration, the filtrate was concentrated under vacuum. The crude residue was applied onto a silica gel column and eluted with dichloromethane/methanol=10:1 to give (3S)-1-[(3,4-difluorophenyl)methyl]-3-(2-isopropylphenyl)piperazine (assumed) as a light yellow oil (55 mg, 42.2%). LC-MS (ESI, m/z) M+1: 331.

Synthesis of 4-{2-[(2S)-4-[(3,4-difluorophenyl)methyl]-2-(2-isopropylphenyl)piperazin-1-yl]-7-azaspiro[3.5]nonan-7-yl}-2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-N-{3-nitro-4-[(oxan-4-ylmethyl)amino]benzenesulfonyl}benzamide (assumed): Into an 8-mL sealed tube, were placed 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-N-{3-nitro-4-[(oxan-4-ylmethyl)amino]benzenesulfonyl}-4-{2-oxo-7-azaspiro[3.5]nonan-7-yl}benzamide (70 mg, 0.09 mmol, 1.0 eq), (3S)-1-[(3,4-difluorophenyl)methyl]-3-(2-isopropylphenyl)piperazine (assumed) (29 mg, 0.09 mmol, 1.0 eq), ZnCl₂ (36 mg, 0.3 mmol, 3.0 eq), NaBH₃CN (17 mg, 0.3 mmol, 3.0 eq), MeOH (1 mL). The resulting solution was stirred for 4 hours at 70° C. The resulting mixture was then quenched by the addition of water (30 mL), and extracted with dichloromethane (2×30 mL). The combined organic layers was washed with brine (2×30 mL), dried over anhydrous Na₂SO₄. After filtration, the filtrate was concentrated under vacuum. The crude residue was applied onto a silica gel column and eluted with dichloromethane/methanol=20:1 to give 4-{2-[(2S)-4-[(3,4-difluorophenyl)methyl]-2-(2-isopropylphenyl)piperazin-1-yl]-7-azaspiro[3.5]nonan-7-yl}-2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-N-{3-nitro-4-[(oxan-4-ylmethyl)amino]benzenesulfonyl}benzamide (assumed) as a yellow solid (10 mg, 10.2%). LC-MS (ESI, m/z) M+1: 1100. ¹H NMR (400 MHz, Methanol-d₄) δ 8.52-8.47 (m, 1H), 7.88-7.80 (m, 1H), 7.53-7.04 (m, 9H), 6.97-6.85 (m, 1H), 6.72-6.62 (m, 2H), 6.56-6.47 (m, 1H), 6.04-5.92 (m, 1H), 4.47-4.21 (m, 1H), 4.04-3.88 (m, 3H), 3.87-3.62 (m, 3H), 3.59-3.42 (m, 6H), 3.30-3.23 (m, 2H), 3.17-2.83 (m, 6H), 2.64-2.56 (m, 1H), 2.47-2.16 (m, 4H), 2.07-1.91 (m, 1H), 1.90-1.81 (m, 1H), 1.78-1.63 (m, 4H), 1.60-1.58 (m, 2H), 1.51-1.37 (m, 4H), 1.33-1.25 (m, 6H), 1.18-1.07 (m, 3H), 0.96-0.83 (m, 1H).

### Example 167 Preparation of 4-{2-[(2R)-4-[(3,4-difluorophenyl)methyl]-2-(2-methylphenyl)piperazin-1-yl]-7-azaspiro[3.5]nonan-7-yl}-2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-N-[(3S)-5-nitro-3-(oxan-4-yl)-3,4-dihydro-2H-1,4-benzoxazin-7-ylsulfonyl]benzamide (assumed)

Synthesis of (3R)-1-[(3,4-difluorophenyl)methyl]-3-(2-methylphenyl)piperazine: Into an 8-mL sealed tube, were placed 3,4-difluorobenzaldehyde (200 mg, 1.4 mmol, 1.0 eq), (2R)-2-(2-methylphenyl)piperazine (248 mg, 1.4 mmol, 1.0 eq), NaBH₃CN (265 mg, 4.2 mmol, 3.0 eq), ZnCl₂ (575 mg, 4.2 mmol, 3.0 eq), MeOH (2 mL). The resulting solution was stirred for 2 hours at 25° C. The resulting mixture was then quenched by the addition of water (30 mL), and extracted with dichloromethane (2×30 mL). The combined organic layers was washed with brine (2×30 mL). The mixture was dried over anhydrous Na₂SO₄. After filtration, the filtrate was concentrated under vacuum. The crude residue was applied onto a silica gel column and eluted with dichloromethane/methanol=10:1 to give (3R)-1-[(3,4-difluorophenyl)methyl]-3-(2-methylphenyl)piperazine as a white solid (260 mg, 61.1%). LC-MS (ESI, m/z) M+1: 303.

Synthesis of methyl 4-{2-[(2R)-4-[(3,4-difluorophenyl)methyl]-2-(2-methylphenyl)piperazin-1-yl]-7-azaspiro[3.5]nonan-7-yl}-2-[(3R,8S)-15-{[2-(trimethylsilyl)ethoxy]methyl}-2,5-dioxa-9,15,17-triazatetracyclo[8.7.0.0\hat{ }{3,8}.0\hat{ }{12,16}]heptadeca-1(10),11,13,16-tetraen-9-yl]benzoate: Into an 8-mL sealed tube, were placed methyl 4-{2-oxo-7-azaspiro[3.5]nonan-7-yl}-2-[(3R,8S)-15-{[2-(trimethylsilyl)ethoxy]methyl}-2,5-dioxa-9,15,17-triazatetracyclo[8.7.0.0\hat{ }{3,8}.0\hat{ }{12,16}]heptadeca-1(10),11,13,16-tetraen-9-yl]benzoate (300 mg, 0.5 mmol, 1.0 eq), (3R)-1-[(3,4-difluorophenyl)methyl]-3-(2-methylphenyl)piperazine (215 mg, 0.7 mmol, 1.5 eq), ZnCl₂ (194 mg, 1.4 mmol, 3.0 eq), NaBH₃CN (89 mg, 1.4 mmol, 3.0 eq), MeOH:DCE=1:1 (3 mL). The resulting solution was stirred for overnight at 50° C. The resulting mixture was then quenched by the addition of water (30 mL) and extracted with dichloromethane (2×30 mL). The combined organic layer was washed with brine (2×30 mL), dried over anhydrous Na₂SO₄. After filtration, the filtrate was concentrated under vacuum. The crude residue was applied onto a silica gel column and eluted with dichloromethane/methanol=10:1 to give methyl 4-{2-[(2R)-4-[(3,4-difluorophenyl)methyl]-2-(2-methylphenyl)piperazin-1-yl]-7-azaspiro[3.5]nonan-7-yl}-2-[(3R,8S)-15-{[2-(trimethylsilyl)ethoxy]methyl}-2,5-dioxa-9,15,17-triazatetracyclo[8.7.0.0\hat{ }{3,8}.0\hat{ }{12,16}]heptadeca-1(10),11,13,16-tetraen-9-yl]benzoate as a light yellow solid (300 mg, 68.9%). LC-MS (ESI, m/z) M+1: 919.

Synthesis of 4-{2-[(2R)-4-[(3,4-difluorophenyl)methyl]-2-(2-methylphenyl)piperazin-1-yl]-7-azaspiro[3.5]nonan-7-yl}-2-[(3R,8S)-15-{[2-(trimethylsilyl)ethoxy]methyl}-2,5-dioxa-9,15,17-triazatetracyclo[8.7.0.0\hat{ }{3,8}.0\hat{ }{12,16}]heptadeca-1(10),11,13,16-tetraen-9-yl]benzoic acid: Into an 8 mL seal tube, were placed methyl 4-{2-[(2R)-4-[(3,4-difluorophenyl)methyl]-2-(2-methylphenyl)piperazin-1-yl]-7-azaspiro[3.5]nonan-7-yl}-2-[(3R,8S)-15-{[2-(trimethylsilyl)ethoxy]methyl}-2,5-dioxa-9,15,17-triazatetracyclo[8.7.0.0\hat{ }{3,8}.0\hat{ }{12,16}]heptadeca-1(10),11,13,16-tetraen-9-yl]benzoate (300 mg, 0.3 mmol, 1.0 eq), 1,4-dioxane:MeOH (1:1, 3 mL), NaOH (52 mg, 1.3 mmol, 4.0 eq). The reaction mixture was stirred for overnight at 50° C. The resulting mixture was then diluted with water (30 mL) and acidified to pH=5 with HCl (1 M). The precipitated solids were collected by filtration and dried under infrared light to give 4-{2-[(2R)-4-[(3,4-difluorophenyl)methyl]-2-(2-methylphenyl)piperazin-1-yl]-7-azaspiro[3.5]nonan-7-yl}-2-[(3R,8S)-15-{[2-(trimethylsilyl)ethoxy]methyl}-2,5-dioxa-9,15,17-triazatetracyclo[8.7.0.0\hat{ }{3,8}.0\hat{ }{12,16}]heptadeca-1(10),11,13,16-tetraen-9-yl]benzoic acid as a light yellow solid (260 mg, 88.0%). LC-MS (ESI, m/z) M+1: 905.

Synthesis of 4-{2-[(2R)-4-[(3,4-difluorophenyl)methyl]-2-(2-methylphenyl)piperazin-1-yl]-7-azaspiro[3.5]nonan-7-yl}-N-[(3S)-5-nitro-3-(oxan-4-yl)-3,4-dihydro-2H-1,4-benzoxazin-7-ylsulfonyl]-2-[(3R,8S)-15-{[2-(trimethylsilyl)ethoxy]methyl}-2,5-dioxa-9,15,17-triazatetracyclo[8.7.0.0\hat{ }{3,8}.0\hat{ }{12,16}]heptadeca-1(10),11,13,16-tetraen-9-yl]benzamide (assumed): Into an 8 mL vial, were placed 4-{2-[(2R)-4-[(3,4-difluorophenyl)methyl]-2-(2-methylphenyl)piperazin-1-yl]-7-azaspiro[3.5]nonan-7-yl}-2-[(3R,8S)-15-{[2-(trimethylsilyl)ethoxy]methyl}-2,5-dioxa-9,15,17-triazatetracyclo[8.7.0.0\hat{ }{3,8}.0\hat{ }{12,16}]heptadeca-1(10),11,13,16-tetraen-9-yl]benzoic acid (80 mg, 0.1 mmol, 1.0 eq), (3S)-5-nitro-3-(oxan-4-yl)-3,4-dihydro-2H-1,4-benzoxazine-7-sulfonamide (assumed) (24 mg, 0.07 mmol, 0.8 eq), dichloromethane (1 mL), EDCl (34 mg, 0.2 mmol, 2.0 eq), DMAP (11 mg, 0.1 mmol, 1.0 eq). The reaction mixture was stirred for 4 hours at 25° C. The resulting mixture was then quenched by the addition of water (50 mL), and extracted with dichloromethane (2×50 mL). The combined organic layers was washed with brine (2×50 mL), dried over anhydrous Na₂SO₄. The resulting mixture was concentrated under vacuum. The crude residue was applied onto a silica gel column and eluted with dichloromethane/methanol=20:1 to give 4-{2-[(2R)-4-[(3,4-difluorophenyl)methyl]-2-(2-methylphenyl)piperazin-1-yl]-7-azaspiro[3.5]nonan-7-yl}-N-[(3S)-5-nitro-3-(oxan-4-yl)-3,4-dihydro-2H-1,4-benzoxazin-7-ylsulfonyl]-2-[(3R,8S)-15-{[2-(trimethylsilyl)ethoxy]methyl}-2,5-dioxa-9,15,17-triazatetracyclo[8.7.0.0\hat{ }{3,8}.0\hat{ }{12,16}]heptadeca-1(10),11,13,16-tetraen-9-yl]benzamide (assumed) as a yellow solid (40 mg, 36.9%). LC-MS (ESI, m/z) M+1: 1231.

Synthesis of 4-{2-[(2R)-4-[(3,4-difluorophenyl)methyl]-2-(2-methylphenyl)piperazin-1-yl]-7-azaspiro[3.5]nonan-7-yl}-2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-N-[(3S)-5-nitro-3-(oxan-4-yl)-3,4-dihydro-2H-1,4-benzoxazin-7-ylsulfonyl]benzamide (assumed): Into an 8-mL sealed tube, were placed 4-{2-[(2R)-4-[(3,4-difluorophenyl)methyl]-2-(2-methylphenyl)piperazin-1-yl]-7-azaspiro[3.5]nonan-7-yl}-N-[(3S)-5-nitro-3-(oxan-4-yl)-3,4-dihydro-2H-1,4-benzoxazin-7-ylsulfonyl]-2-[(3R,8S)-15-{[2-(trimethylsilyl)ethoxy]methyl}-2,5-dioxa-9,15,17-triazatetracyclo[8.7.0.0\hat{ }{3,8}.0\hat{ }{12,16}]heptadeca-1(10),11,13,16-tetraen-9-yl]benzamide (assumed) (40 mg, 0.03 mmol, 1.0 eq), ethylenediamine (39 mg, 0.7 mmol, 20.0 eq), TBAF in THE (1 M, 1 mL). The resulting solution was stirred for overnight at 80° C. The resulting mixture was then quenched by the addition of water (200 mL) and extracted with ethyl acetate (2×100 mL). The combined organic layers was washed with brine (2×100 mL), dried over anhydrous Na₂SO₄. After filtration, the filtrate was concentrated under vacuum. The crude residue was applied onto a silica gel column and eluted with dichloromethane/methanol=20:1 to give 4-{2-[(2R)-4-[(3,4-difluorophenyl)methyl]-2-(2-methylphenyl)piperazin-1-yl]-7-azaspiro[3.5]nonan-7-yl}-2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-N-[(3S)-5-nitro-3-(oxan-4-yl)-3,4-dihydro-2H-1,4-benzoxazin-7-ylsulfonyl]benzamide (assumed) as a yellow solid (10 mg, 28.0%). LC-MS (ESI, m/z) M+1: 1100. ¹H NMR (400 MHz, Methanol-d₄) δ 8.13-7.82 (m, 2H), 7.62-7.36 (m, 2H), 7.34-7.08 (m, 6H), 7.07-7.04 (m, 1H), 7.00-6.86 (m, 1H), 6.82-6.65 (m, 1H), 6.55-6.43 (m, 1H), 6.08-5.85 (m, 1H), 4.33-4.19 (m, 2H), 4.13-4.06 (m, 1H), 4.04-3.95 (m, 2H), 3.95-3.87 (m, 1H), 3.77-3.63 (m, 2H), 3.62-3.39 (m, 4H), 3.23-3.00 (m, 6H), 2.99-2.88 (m, 1H), 2.73-2.60 (m, 1H), 2.41-2.21 (m, 5H), 1.97-1.83 (m, 2H), 1.82-1.38 (m, 9H), 1.37-1.24 (m, 6H), 1.20-1.11 (m, 1H), 0.96-0.89 (m, 2H).

### Example 168 Preparation of 4-{2-[(2R)-4-[(3,4-difluorophenyl)methyl]-2-(2-methylphenyl)piperazin-1-yl]-7-azaspiro[3.5]nonan-7-yl}-2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-N-[(3R)-5-nitro-3-(oxan-4-yl)-3,4-dihydro-2H-1,4-benzoxazin-7-ylsulfonyl]benzamide

Synthesis of 4-{2-[(2R)-4-[(3,4-difluorophenyl)methyl]-2-(2-methylphenyl)piperazin-1-yl]-7-azaspiro[3.5]nonan-7-yl}-N-[(3R)-5-nitro-3-(oxan-4-yl)-3,4-dihydro-2H-1,4-benzoxazin-7-ylsulfonyl]-2-[(3R,8S)-15-{[2-(trimethylsilyl)ethoxy]methyl}-2,5-dioxa-9,15,17-triazatetracyclo[8.7.0.0\hat{ }{3,8}.0\hat{ }{12,16}]heptadeca-1(10),11,13,16-tetraen-9-yl]benzamide (assumed): Into an 8 mL vial, were placed 4-{2-[(2R)-4-[(3,4-difluorophenyl)methyl]-2-(2-methylphenyl)piperazin-1-yl]-7-azaspiro[3.5]nonan-7-yl}-2-[(3R,8S)-15-{[2-(trimethylsilyl)ethoxy]methyl}-2,5-dioxa-9,15,17-triazatetracyclo[8.7.0.0\hat{ }{3,8}.0\hat{ }{12,16}]heptadeca-1(10),11,13,16-tetraen-9-yl]benzoic acid (80 mg, 0.08 mmol, 1.0 eq), (3R)-5-nitro-3-(oxan-4-yl)-3,4-dihydro-2H-1,4-benzoxazine-7-sulfonamide (assumed) (24 mg, 0.07 mmol, 0.8 eq), dichloromethane (1 mL), EDCl (34 mg, 0.17 mmol, 2.0 eq), DMAP (11 mg, 0.09 mmol, 1.0 eq). The reaction mixture was stirred for 4 hours at 25° C. The resulting mixture was then quenched by the addition of water (50 mL), and extracted with dichloromethane (2×50 mL). The combined organic layers was washed with brine (2×50 mL), dried over anhydrous Na₂SO₄. The resulting mixture was concentrated under vacuum. The crude residue was applied onto a silica gel column and eluted with dichloromethane/methanol=20:1 to give 4-{2-[(2R)-4-[(3,4-difluorophenyl)methyl]-2-(2-methylphenyl)piperazin-1-yl]-7-azaspiro[3.5]nonan-7-yl}-N-[(3R)-5-nitro-3-(oxan-4-yl)-3,4-dihydro-2H-1,4-benzoxazin-7-ylsulfonyl]-2-[(3R,8S)-15-{[2-(trimethylsilyl)ethoxy]methyl}-2,5-dioxa-9,15,17-triazatetracyclo[8.7.0.0\hat{ }{3,8}.0\hat{ }{12,16}]heptadeca-1(10),11,13,16-tetraen-9-yl]benzamide (assumed) as a yellow solid (40 mg, 36.9%). LC-MS (ESI, m/z) M+1: 1231.

Synthesis of 4-{2-[(2R)-4-[(3,4-difluorophenyl)methyl]-2-(2-methylphenyl)piperazin-1-yl]-7-azaspiro[3.5]nonan-7-yl}-2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-N-[(3R)-5-nitro-3-(oxan-4-yl)-3,4-dihydro-2H-1,4-benzoxazin-7-ylsulfonyl]benzamide (assumed): Into an 8-mL sealed tube, were placed 4-{2-[(2R)-4-[(3,4-difluorophenyl)methyl]-2-(2-methylphenyl)piperazin-1-yl]-7-azaspiro[3.5]nonan-7-yl}-N-[(3R)-5-nitro-3-(oxan-4-yl)-3,4-dihydro-2H-1,4-benzoxazin-7-ylsulfonyl]-2-[(3R,8S)-15-{[2-(trimethylsilyl)ethoxy]methyl}-2,5-dioxa-9,15,17-triazatetracyclo[8.7.0.0\hat{ }{3,8}.0\hat{ }{12,16}]heptadeca-1(10),11,13,16-tetraen-9-yl]benzamide (assumed) (40 mg, 0.03 mmol, 1.0 eq), ethylenediamine (39 mg, 0.7 mmol, 20.0 eq), TBAF in THE (1 M, 1 mL). The resulting solution was stirred for overnight at 80° C. The resulting mixture was then quenched by the addition of water (200 mL), and extracted with ethyl acetate (2×100 mL). The combined organic layers was washed with brine (2×100 mL), dried over anhydrous Na₂SO₄. After filtration, the filtrate was concentrated under vacuum. The crude residue was applied onto a silica gel column and eluted with dichloromethane/methanol=20:1 to give 4-{2-[(2R)-4-[(3,4-difluorophenyl)methyl]-2-(2-methylphenyl)piperazin-1-yl]-7-azaspiro[3.5]nonan-7-yl}-2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-N-[(3R)-5-nitro-3-(oxan-4-yl)-3,4-dihydro-2H-1,4-benzoxazin-7-ylsulfonyl]benzamide (assumed) as a yellow solid (10 mg, 28.0%). LC-MS (ESI, m/z) M+1: 1100. ¹H NMR (400 MHz, Methanol-d₄) δ 8.13-7.82 (m, 2H), 7.62-7.36 (m, 2H), 7.34-7.08 (m, 6H), 7.07-7.04 (m, 1H), 7.00-6.86 (m, 1H), 6.82-6.65 (m, 1H), 6.55-6.43 (m, 1H), 6.08-5.85 (m, 1H), 4.33-4.19 (m, 2H), 4.13-4.06 (m, 1H), 4.04-3.95 (m, 2H), 3.95-3.87 (m, 1H), 3.77-3.63 (m, 2H), 3.62-3.39 (m, 4H), 3.23-3.00 (m, 6H), 2.99-2.88 (m, 1H), 2.73-2.60 (m, 1H), 2.41-2.21 (m, 5H), 1.97-1.83 (m, 2H), 1.82-1.38 (m, 9H), 1.37-1.24 (m, 6H), 1.20-1.11 (m, 1H), 0.96-0.89 (m, 2H).

### Example 169 Preparation of 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-4-{2-[(2R)-2-(2-isopropoxyphenyl)-4-({5-methoxy-6-[(3S)-3-methylmorpholin-4-yl]pyridin-3-yl}methyl)piperazin-1-yl]-7-azaspiro[3.5]nonan-7-yl}-N-[(3R)-5-nitro-3-(oxan-4-yl)-3,4-dihydro-2H-1,4-benzoxazin-7-ylsulfonyl]benzamide (assumed)

Synthesis of (3S)-4-(5-{[(3R)-3-(2-isopropoxyphenyl)piperazin-1-yl]methyl}-3-methoxypyridin-2-yl)-3-methylmorpholine (assumed): Into a 40 mL vial were added 5-methoxy-6-[(3S)-3-methylmorpholin-4-yl]pyridine-3-carbaldehyde (120 mg, 0.5 mmol, 1.0 eq), (2R)-2-[2-(methyl-lambda3-oxy)phenyl]piperazine (assumed) (120 mg, 0.5 mmol, 1.0 eq), NaBH₃CN (160 mg, 2.5 mmol, 5.0 eq), ZnCl₂ (346 mg, 2.5 mmol, 5.0 eq) and MeOH (3 mL). The resulting mixture was stirred for 2 hours at 25° C. The reaction was quenched with water (2 mL), and extracted with CH₂Cl₂ (3×5 mL). The combined organic layers were washed with brine (5 mL), dried over anhydrous Na₂SO₄. After filtration, the filtrate was concentrated under vacuum. The residue was purified by Prep-TLC (CH₂Cl₂/MeOH=10:1) to give (3S)-4-(5-{[(3R)-3-(2-isopropoxyphenyl)piperazin-1-yl]methyl}-3-methoxypyridin-2-yl)-3-methylmorpholine (assumed) as an off-white solid (100 mg, 44.7%). LC-MS (ESI, m/z) M+1: 441.

Synthesis of 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-4-{2-[(2R)-2-(2-isopropoxyphenyl)-4-({5-methoxy-6-[(3S)-3-methylmorpholin-4-yl]pyridin-3-yl}methyl)piperazin-1-yl]-7-azaspiro[3.5]nonan-7-yl}-N-[(3R)-5-nitro-3-(oxan-4-yl)-3,4-dihydro-2H-1,4-benzoxazin-7-ylsulfonyl]benzamide (assumed): Into an 8 mL vial were added (3S)-4-(5-{[(3R)-3-(2-isopropoxyphenyl)piperazin-1-yl]methyl}-3-methoxypyridin-2-yl)-3-methylmorpholine (assumed) (30 mg, 0.07 mmol, 1.0 eq), 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-N-[(3R)-5-nitro-3-(oxan-4-yl)-3,4-dihydro-2H-1,4-benzoxazin-7-ylsulfonyl]-4-{2-oxo-7-azaspiro[3.5]nonan-7-yl}benzamide (55 mg, 0.07 mmol, 1.0 eq), NaBH₃CN (21 mg, 0.3 mmol, 5.0 eq), ZnCl₂ (46 mg, 0.3 mmol, 5.0 eq) and MeOH (1 mL). The resulting mixture was stirred for overnight at 70° C. The reaction was quenched by the addition of water (3 mL). The resulting mixture was extracted with CH₂Cl₂ (3×5 mL). The combined organic layers were washed with brine (5 mL), dried over anhydrous Na₂SO₄. After filtration, the filtrate was concentrated under vacuum. The residue was purified by Prep-TLC (CH₂Cl₂/MeOH=15:1) to give 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-4-{2-[(2R)-2-(2-isopropoxyphenyl)-4-({5-methoxy-6-[(3S)-3-methylmorpholin-4-yl]pyridin-3-yl}methyl)piperazin-1-yl]-7-azaspiro[3.5]nonan-7-yl}-N-[(3R)-5-nitro-3-(oxan-4-yl)-3,4-dihydro-2H-1,4-benzoxazin-7-ylsulfonyl]benzamide as a yellow solid (20 mg, 23.7%). LC-MS (ESI, m/z) M+1: 1239. ¹H NMR (400 MHz, Chloroform-d) δ 12.63 (s, 1H), 8.45 (s, 1H), 8.37 (s, 1H), 8.31 (d, J=2.1 Hz, 1H), 8.12 (d, J=9.2 Hz, 1H), 7.89-7.74 (m, 1H), 7.55-7.40 (m, 2H), 7.15 (t, J=8.0 Hz, 1H), 7.09 (t, J=3.0 Hz, 1H), 6.84 (dt, J=15.4, 8.2 Hz, 3H), 6.70 (s, 1H), 6.43 (s, 1H), 6.19-6.09 (m, 1H), 4.59-4.48 (m, 3H), 4.45-4.41 (m, 1H), 4.13-4.12 (m, 2H), 4.11-4.02 (m, 2H), 4.02-3.94 (m, 1H), 3.90-3.92 (m, 2H), 3.85 (s, 3H), 3.87-3.79 (m, 1H), 3.67-3.55 (m, 2H), 3.48-3.45 (m, 4H), 3.42-3.40 (m, 4H), 3.20-3.15 (m, 2H), 3.10-3.08 (m, 4H), 2.93-2.88 (m, 3H), 2.63-2.56 (m, 1H), 2.42-2.10 (m, 3H), 1.90-1.74 (m, 4H), 1.56-1.43 (m, 6H), 1.43-1.35 (m, 3H), 1.32-1.24. (m, 4H), 1.19-1.07 (m, 4H), 1.05-0.89 (m, 2H).

### Example 170 Preparation of 4-{2-[(2R)-4-[(4S)-6,7-difluoro-3,4-dihydro-2H-1-benzopyran-4-yl]-2-(2-methylphenyl)piperazin-1-yl]-7-azaspiro[3.5]nonan-7-yl}-2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-N-[(3R)-5-nitro-3-(oxan-4-yl)-3,4-dihydro-2H-1,4-benzoxazin-7-ylsulfonyl]benzamide (assumed)

Synthesis of 3-(3,4-difluorophenoxy)propanoic acid: Into a 250 mL round-bottom flask were added 3,4-difluorophenol (10.0 g, 76.9 mmol, 1.0 eq), propanoic acid, 3-bromo-(13.0 g, 85.3 mmol, 1.1 eq), NaOH (6.2 g, 155.3 mmol, 2.0 eq) and water (100 mL). The resulting mixture was stirred for overnight at 100° C. The mixture was acidified to pH=2 with HCl (3 M). The resulting mixture was diluted with water (30 mL), and extracted with EtOAc (3×50 mL). The combined organic layers were washed with brine (50 mL), dried over anhydrous Na₂SO₄. After filtration, the filtrate was concentrated under vacuum. The residue was applied onto a silica gel column and eluted with ethyl acetate/petroleum ether=1:3 to give 3-(3,4-difluorophenoxy)propanoic acid as a colorless solid (4.1 g, 26.3%). ¹H NMR (300 MHz, Chloroform-d) δ 9.46 (s, 1H), 7.08 (dt, J=10.1, 9.1 Hz, 1H), 6.76 (ddd, J=11.9, 6.6, 3.0 Hz, 1H), 6.63 (dtd, J=9.1, 3.2, 1.7 Hz, 1H), 4.22 (t, J=6.2 Hz, 2H), 2.87 (t, J=6.2 Hz, 2H).

Synthesis of 6,7-difluoro-2,3-dihydro-1-benzopyran-4-one: Into a 100 mL round-bottom flask were added 3-(3,4-difluorophenoxy)propanoic acid (3.5 g, 17.3 mmol, 1.0 eq) and trifluoromethanesulfonic acid (30 mL) at 0° C. The resulting mixture was stirred for 18 hours at 25° C. The reaction was quenched by the addition of water/ice (50 mL) at 0° C. The precipitated solids were collected by filtration and washed with water (3×10 mL). Finally, 6,7-difluoro-2,3-dihydro-1-benzopyran-4-one was obtained as an off-white solid (2.2 g, 69.0%). ¹H NMR (300 MHz, Chloroform-d) δ 7.70 (dd, J=10.0, 9.0 Hz, 1H), 6.81 (dd, J=10.8, 6.3 Hz, 1H), 4.65-4.50 (m, 2H), 2.94-2.68 (m, 2H).

Synthesis of 6,7-difluoro-3,4-dihydro-2H-1-benzopyran-4-ol: To a solution of 6,7-difluoro-2,3-dihydro-1-benzopyran-4-one (1.5 g, 8.1 mmol, 1.0 eq) in MeOH (10 mL) was added NaBH₄ (0.9 g, 24.4 mmol, 3.0 eq) in portions at 0° C. The resulting mixture was stirred for 2 hours at 25° C. The reaction was quenched by the addition of water (5 mL) and extracted with EtOAc (3×10 mL). The combined organic layers were washed with brine (10 mL), dried over anhydrous Na₂SO₄. After filtration, the filtrate was concentrated under vacuum to give 6,7-difluoro-3,4-dihydro-2H-1-benzopyran-4-ol as a white solid (1.3 g, 85.7%). ¹H NMR (300 MHz, Chloroform-d) δ 7.14 (dd, J=10.4, 8.8 Hz, 1H), 6.66 (dd, J=11.5, 6.9 Hz, 1H), 4.75 (t, J=4.4 Hz, 1H), 4.34-4.19 (m, 2H), 2.20-1.99 (m, 2H), 1.81 (s, 1H).

Synthesis of 4-chloro-6,7-difluoro-3,4-dihydro-2H-1-benzopyran: To a solution of 6,7-difluoro-3,4-dihydro-2H-1-benzopyran-4-ol (750 mg, 4.0 mmol, 1.0 eq) in CHCl₃ (5 mL) was added SOCl₂ (5 mL, 68.9 mmol, 17.1 eq) dropwise at 0° C. The resulting mixture was stirred for 4 hours at 25° C. The resulting mixture was concentrated under vacuum. The resulting mixture was diluted with water (10 mL), and extracted with EtOAc (3×10 mL). The combined organic layers were washed with brine (10 mL), dried over anhydrous Na₂SO₄. After filtration, the filtrate was concentrated under vacuum to afford 4-chloro-6,7-difluoro-3,4-dihydro-2H-1-benzopyran as an orange oil (610 mg, 74.0%). ¹H NMR (300 MHz, Chloroform-d) δ 7.11 (dd, J=10.3, 8.6 Hz, 1H), 6.65 (dd, J=11.3, 6.9 Hz, 1H), 5.15 (t, J=3.7 Hz, 1H), 4.53-4.25 (m, 2H), 2.59-2.38 (m, 1H), 2.31 (dq, J=15.0, 3.0 Hz, 1H).

Synthesis of (3R)-1-[(4S)-6,7-difluoro-3,4-dihydro-2H-1-benzopyran-4-yl]-3-(2-methylphenyl)piperazine (assumed) and (3R)-1-[(4R)-6,7-difluoro-3,4-dihydro-2H-1-benzopyran-4-yl]-3-(2-methylphenyl)piperazine (assumed): Into a 40 mL vial were added 4-chloro-6,7-difluoro-3,4-dihydro-2H-1-benzopyran (600 mg, 2.9 mmol, 1.0 eq), (2R)-2-(2-methylphenyl)piperazine (517 mg, 2.9 mmol, 1.0 eq), K₂CO₃ (1216 mg, 8.8 mmol, 3.0 eq) and MeCN (8 mL). The resulting mixture was stirred for overnight at 80° C. The resulting mixture was diluted with water (10 mL) and extracted with EtOAc (3×10 mL). The combined organic layers were washed with brine (10 mL), dried over anhydrous Na₂SO₄. After filtration, the filtrate was concentrated under vacuum. The residue was purified by Prep-TLC (EA 100%) to give (3R)-1-[(4S)-6,7-difluoro-3,4-dihydro-2H-1-benzopyran-4-yl]-3-(2-methylphenyl)piperazine (assumed) (35 mg, 3.5%) and (3R)-1-[(4R)-6,7-difluoro-3,4-dihydro-2H-1-benzopyran-4-yl]-3-(2-methylphenyl)piperazine (assumed) as a colorless solid (180 mg, 17.8%). LC-MS (ESI, m/z) M+1: 345.

Synthesis of 4-{2-[(2R)-4-[(4S)-6,7-difluoro-3,4-dihydro-2H-1-benzopyran-4-yl]-2-(2-methylphenyl)piperazin-1-yl]-7-azaspiro[3.5]nonan-7-yl}-2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-N-[(3R)-5-nitro-3-(oxan-4-yl)-3,4-dihydro-2H-1,4-benzoxazin-7-ylsulfonyl]benzamide (assumed): Into an 8 mL vial were added (3R)-1-[(4S)-6,7-difluoro-3,4-dihydro-2H-1-benzopyran-4-yl]-3-(2-methylphenyl)piperazine (assumed) (20 mg, 0.06 mmol, 1.0 eq), 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-N-[(3R)-5-nitro-3-(oxan-4-yl)-3,4-dihydro-2H-1,4-benzoxazin-7-ylsulfonyl]-4-{2-oxo-7-azaspiro[3.5]nonan-7-yl}benzamide (47 mg, 0.06 mmol, 1.0 eq), NaBH₃CN (18 mg, 0.3 mmol, 5.0 eq), ZnCl₂ (40 mg, 0.3 mmol, 5.0 eq) and MeOH (3 mL). The resulting mixture was stirred for overnight at 70° C. The reaction was quenched by the addition of water (3 mL), and extracted with CH₂Cl₂ (3×10 mL). The combined organic layers were washed with brine (10 mL), dried over anhydrous Na₂SO₄. After filtration, the filtrate was concentrated under vacuum. The residue was purified by Prep-TLC (CH₂Cl₂/MeOH=15:1) to give 4-{2-[(2R)-4-[(4S)-6,7-difluoro-3,4-dihydro-2H-1-benzopyran-4-yl]-2-(2-methylphenyl)piperazin-1-yl]-7-azaspiro[3.5]nonan-7-yl}-2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-N-[(3R)-5-nitro-3-(oxan-4-yl)-3,4-dihydro-2H-1,4-benzoxazin-7-ylsulfonyl]benzamide (assumed) as a yellow solid (7 mg, 10.6%). LC-MS (ESI, m/z) M+1: 1143. ¹H NMR (400 MHz, Chloroform-d) δ 12.62 (s, 1H), 8.46-8.28 (m, 3H), 8.13 (d, J=9.1 Hz, 1H), 7.46 (s, 2H), 7.39 (s, 1H), 7.23-7.05 (m, 3H), 6.86 (s, 1H), 6.65 (d, J=30.4 Hz, 3H), 6.44 (s, 1H), 6.14 (s, 1H), 4.58-4.41 (m, 2H), 4.44-4.32 (m, 2H), 4.13-4.08 (m, 5H), 4.03-3.76 (m, 3H), 3.96-3.58 (m, 2H), 3.45-3.40 (m, 6H), 3.10-3.03 (m, 6H), 2.90-2.56 (m, 5H), 2.56-2.36 (m, 4H), 2.29-1.92 (m, 6H), 1.89-1.71 (m, 5H).

### Example 171 Preparation of 4-{2-[(2R)-4-[(4R)-6,7-difluoro-3,4-dihydro-2H-1-benzopyran-4-yl]-2-(2-methylphenyl)piperazin-1-yl]-7-azaspiro[3.5]nonan-7-yl}-2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-N-[(3R)-5-nitro-3-(oxan-4-yl)-3,4-dihydro-2H-1,4-benzoxazin-7-ylsulfonyl]benzamide (assumed)

Synthesis of 4-{2-[(2R)-4-[(4R)-6,7-difluoro-3,4-dihydro-2H-1-benzopyran-4-yl]-2-(2-methylphenyl)piperazin-1-yl]-7-azaspiro[3.5]nonan-7-yl}-2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-N-[(3R)-5-nitro-3-(oxan-4-yl)-3,4-dihydro-2H-1,4-benzoxazin-7-ylsulfonyl]benzamide (assumed): Into an 8 mL vial were added (3R)-1-[(4R)-6,7-difluoro-3,4-dihydro-2H-1-benzopyran-4-yl]-3-(2-methylphenyl)piperazine (assumed) (20 mg, 0.06 mmol, 1.0 eq), 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-N-[(3R)-5-nitro-3-(oxan-4-yl)-3,4-dihydro-2H-1,4-benzoxazin-7-ylsulfonyl]-4-{2-oxo-7-azaspiro[3.5]nonan-7-yl}benzamide (47 mg, 0.06 mmol, 1.0 eq), NaBH₃CN (18 mg, 0.3 mmol, 5.0 eq), ZnCl₂ (40 mg, 0.3 mmol, 5.0 eq) and MeOH (3 mL). The resulting mixture was stirred for overnight at 70° C. The reaction was quenched by the addition of water (5 mL) and extracted with CH₂Cl₂ (3×10 mL). The combined organic layers were washed with brine (10 mL), dried over anhydrous Na₂SO₄. After filtration, the filtrate was concentrated under vacuum. The residue was purified by Prep-TLC (CH₂Cl₂/MeOH=10:1) to give 4-{2-[(2R)-4-[(4R)-6,7-difluoro-3,4-dihydro-2H-1-benzopyran-4-yl]-2-(2-methylphenyl)piperazin-1-yl]-7-azaspiro[3.5]nonan-7-yl}-2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-N-[(3R)-5-nitro-3-(oxan-4-yl)-3,4-dihydro-2H-1,4-benzoxazin-7-ylsulfonyl]benzamide (assumed) as a yellow solid (7 mg, 10.6%). LC-MS (ESI, m/z) M+1: 1143. ¹H NMR (300 MHz, Chloroform-d) δ 12.61 (s, 1H), 8.42 (d, J=15.5 Hz, 2H), 8.31 (d, J=2.2 Hz, 1H), 8.14 (d, J=9.1 Hz, 1H), 7.54-7.35 (m, 3H), 7.08 (d, J=6.9 Hz, 3H), 6.86 (d, J=9.2 Hz, 1H), 6.70 (s, 1H), 6.54 (dd, J=11.4, 7.0 Hz, 1H), 6.45 (s, 1H), 6.15 (s, 1H), 4.61-4.40 (m, 2H), 4.25-4.15-(m, 1H), 4.13-4.09 (m, 2H), 4.07-4.02 (m, 3H), 3.99-3.88 (m, 1H), 3.86-3.83 (m, 1H), 3.49-3.42 (m, 6H), 3.08-3.04 (m, 4H), 3.03-2.81 (m, 3H), 2.44-2.18 (m, 6H), 2.01-1.94 (m, 3H), 1.88-1.68 (m, 5H), 1.38 (s, 7H), 1.26-1.22 (m, 3H).

### Example 172 Preparation of 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-4-{2-[(2R)-2-(2-isopropylphenyl)-4-({5-methoxy-6-[(3S)-3-methylmorpholin-4-yl]pyridin-3-yl}methyl)piperazin-1-yl]-7-azaspiro[3.5]nonan-7-yl}-N-[(3R)-5-nitro-3-(oxan-4-yl)-3,4-dihydro-2H-1,4-benzoxazin-7-ylsulfonyl]benzamide (assumed)

Synthesis of (3S)-4-(5-{[(3R)-3-(2-isopropylphenyl)piperazin-1-yl]methyl}-3-methoxypyridin-2-yl)-3-methylmorpholine: Into an 8 mL vial were added 5-methoxy-6-[(3S)-3-methylmorpholin-4-yl]pyridine-3-carbaldehyde (100 mg, 0.4 mmol, 1.0 eq), (2R)-2-(2-isopropylphenyl)piperazine (assumed) (86 mg, 0.4 mmol, 1.0 eq), NaBH₃CN (133 mg, 2.1 mmol, 5.0 eq), ZnCl₂ (288 mg, 2.1 mmol, 5.0 eq) and MeOH (3 mL). The resulting mixture was stirred for 2 hours at 25° C. The crude residue was purified by Prep-TLC (CH₂Cl₂/MeOH=10:1) to give (3S)-4-(5-{[(3R)-3-(2-isopropylphenyl)piperazin-1-yl]methyl}-3-methoxypyridin-2-yl)-3-methylmorpholine as a colorless oil (78 mg, 43.4%). LC-MS (ESI, m/z) M+1: 425.

Synthesis of 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-4-{2-[(2R)-2-(2-isopropylphenyl)-4-({5-methoxy-6-[(3S)-3-methylmorpholin-4-yl]pyridin-3-yl}methyl)piperazin-1-yl]-7-azaspiro[3.5]nonan-7-yl}-N-[(3R)-5-nitro-3-(oxan-4-yl)-3,4-dihydro-2H-1,4-benzoxazin-7-ylsulfonyl]benzamide (assumed): Into an 8 mL vial were added (3S)-4-(5-{[(3R)-3-(2-isopropylphenyl)piperazin-1-yl]methyl}-3-methoxypyridin-2-yl)-3-methylmorpholine (assumed) (30 mg, 0.07 mmol, 1.0 eq), 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-N-[(3R)-5-nitro-3-(oxan-4-yl)-3,4-dihydro-2H-1,4-benzoxazin-7-ylsulfonyl]-4-{2-oxo-7-azaspiro[3.5]nonan-7-yl}benzamide (57 mg, 0.07 mmol, 1.0 eq), NaBH₃CN (22 mg, 0.3 mmol, 5.0 eq), ZnCl₂ (48 mg, 0.3 mmol, 5.0 eq) and MeOH (1 mL). The resulting mixture was stirred for overnight at 70° C. The reaction was quenched by the addition of water (2 mL) and extracted with CH₂Cl₂ (3×5 mL). The combined organic layers were washed with brine (5 mL), dried over anhydrous Na₂SO₄. After filtration, the filtrate was concentrated under vacuum. The residue was purified by Prep-TLC (CH₂Cl₂/MeOH=15:1) to give 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-4-{2-[(2R)-2-(2-isopropylphenyl)-4-({5-methoxy-6-[(3S)-3-methylmorpholin-4-yl]pyridin-3-yl}methyl)piperazin-1-yl]-7-azaspiro[3.5]nonan-7-yl}-N-[(3R)-5-nitro-3-(oxan-4-yl)-3,4-dihydro-2H-1,4-benzoxazin-7-ylsulfonyl]benzamide as a yellow solid (16 mg, 18.5%). LC-MS (ESI, m/z) M+1: 1223. ¹H NMR (400 MHz, Chloroform-d) δ 12.63 (s, 1H), 8.52-8.39 (m, 2H), 8.31 (d, J=2.1 Hz, 1H), 8.12 (d, J=9.2 Hz, 1H), 7.76 (s, 1H), 7.46 (d, J=2.1 Hz, 2H), 7.21 (d, J=7.6 Hz, 2H), 7.17-7.03 (m, 2H), 6.85 (dd, J=9.3, 2.5 Hz, 1H), 6.69 (s, 1H), 6.43 (d, J=2.4 Hz, 1H), 6.14 (dd, J=3.5, 2.0 Hz, 1H), 4.64-4.41 (m, 2H), 4.22-4.03 (m, 4H), 4.03-3.90 (m, 2H), 3.85 (s, 3H), 3.79-3.76 (m, 1H), 3.72-3.62 (m, 1H), 3.57-3.44 (m, 4H), 3.40-3.35 (m, 4H), 3.193.16 (m, 1H), 3.15-2.96 (m, 4H), 2.91-2.88 (m, 2H), 2.65-2.54 (m, 1H), 2.43-2.14 (m, 3H), 2.02-1.94 (m, 1H), 1.90-1.70 (m, 5H), 1.54-1.38 (m, 6H), 1.38-1.19 (m, 6H), 1.12-1.05 (m, 8H), 0.97-0.74 (m, 2H).

### Example 173 Preparation of 4-{2-[(2R)-4-[(5,6-dimethoxypyridin-3-yl)methyl]-2-(2-isopropylphenyl)piperazin-1-yl]-7-azaspiro[3.5]nonan-7-yl}-2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-N-[(3R)-5-nitro-3-(oxan-4-yl)-3,4-dihydro-2H-1,4-benzoxazin-7-ylsulfonyl]benzamide (assumed)

Synthesis of (R)-1-((5,6-dimethoxypyridin-3-yl)methyl)-3-(2-isopropylphenyl)piperazine: Into a 40 mL vial were added 5,6-dimethoxypyridine-3-carbaldehyde (123 mg, 0.7 mmol, 1.0 eq), (2R)-2-(2-isopropylphenyl)piperazine (150 mg, 0.7 mmol, 1.0 eq), NaBH(OAc)₃(779 mg, 3.6 mmol, 5.0 eq) and DCE (3 mL). The resulting mixture was stirred for additional 3 hours at 25° C. The reaction was quenched with water, extracted with CH₂Cl₂ (10 mL). The combined organic layers were washed with brine (3×5 mL), dried over anhydrous Na₂SO₄. After filtration, the filtrate was concentrated under vacuum. The crude residue was purified by Prep-TLC (CH₂Cl₂/MeOH=10:1) to give (R)-1-((5,6-dimethoxypyridin-3-yl)methyl)-3-(2-isopropylphenyl)piperazine- as a yellow oil (83 mg, 31.7%). LC-MS (ESI, m/z) M+1: 356.

Synthesis of 4-{2-[(2R)-4-[(5,6-dimethoxypyridin-3-yl)methyl]-2-(2-isopropylphenyl)piperazin-1-yl]-7-azaspiro[3.5]nonan-7-yl}-2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-N-[(3R)-5-nitro-3-(oxan-4-yl)-3,4-dihydro-2H-1,4-benzoxazin-7-ylsulfonyl]benzamide (assumed): Into an 8 mL vial were added (3R)-1-[(5,6-dimethoxypyridin-3-yl)methyl]-3-(2-isopropylphenyl)piperazine (30 mg, 0.08 mmol, 1.0 eq), 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-N-[(3R)-5-nitro-3-(oxan-4-yl)-3,4-dihydro-2H-1,4-benzoxazin-7-ylsulfonyl]-4-{2-oxo-7-azaspiro[3.5]nonan-7-yl}benzamide (69 mg, 0.08 mmol, 1.0 eq), NaBH₃CN (26.52 mg, 0.420 mmol, 5.0 eq), ZnCl₂ (27.59 mg, 0.420 mmol, 5.0 eq) and MeOH (2 mL). The reaction was quenched by the addition of water (5 mL), and extracted with CH₂Cl₂ (3×10 mL). The combined organic layers were washed with brine (5 mL), dried over anhydrous Na₂SO₄. After filtration, the filtrate was concentrated under reduced pressure. The residue was purified by Prep-TLC (CH₂Cl₂/MeOH=15:1) to give 4-{2-[(2R)-4-[(5,6-dimethoxypyridin-3-yl)methyl]-2-(2-isopropylphenyl)piperazin-1-yl]-7-azaspiro[3.5]nonan-7-yl}-2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-N-[(3R)-5-nitro-3-(oxan-4-yl)-3,4-dihydro-2H-1,4-benzoxazin-7-ylsulfonyl]benzamide (assumed) as a yellow solid (13 mg, 13.4%). LC-MS (ESI, m/z) M+1: 1239.5. ¹H NMR (400 MHz, Chloroform-d) δ 12.63 (s, 1H), 8.45 (s, 1H), 8.37 (s, 1H), 8.31 (d, J=2.1 Hz, 1H), 8.12 (d, J=9.2 Hz, 1H), 7.89-7.74 (m, 1H), 7.55-7.40 (m, 2H), 7.15 (t, J=8.0 Hz, 1H), 7.09 (t, J=3.0 Hz, 1H), 6.84 (dt, J=15.4, 8.2 Hz, 3H), 6.70 (s, 1H), 6.43 (s, 1H), 6.19-6.09 (m, 1H), 4.66-4.64 (m, 1H), 4.59-4.48 (m, 2H), 4.45-4.42 (m, 1H), 4.13-4.12 (m, 2H), 4.11-4.02 (m, 2H), 4.02-3.94 (m, 1H), 3.90-3.87 (m, 2H), 3.85 (s, 3H), 3.79-3.69 (m, 1H), 3.67-3.55 (m, 2H), 3.49-3.46 (m, 4H), 3.40-3.36 (m, 4H), 3.20-3.12 (m, 2H), 3.08-3.01 (m, 4H), 2.93-2.87 (m, 3H), 2.63-2.55 (m, 1H), 2.42-2.10 (m, 3H), 1.90-1.74 (m, 4H), 1.56-1.43 (m, 6H), 1.43-1.35 (m, 3H), 1.32-1.26 (m, 4H).

### Example 174 Preparation of 4-(2-((R)-2-(2-isopropoxyphenyl)-4-((R)-tetrahydro-2H-pyran-3-yl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide (assumed)

Synthesis of (R)-3-(2-isopropoxyphenyl)-1-((R)-tetrahydro-2H-pyran-3-yl)piperazine and (R)-3-(2-isopropoxyphenyl)-1-((S)-tetrahydro-2H-pyran-3-yl)piperazine: Into a 40 mL vial were added oxan-3-one (272 mg, 2.7 mmol, 1.0 eq), (2R)-2-(2-isopropoxyphenyl)piperazine (598 mg, 2.7 mmol, 1.0 eq), NaBH₃CN (853 mg, 13.5 mmol, 5.0 eq), ZnCl₂ (1851 mg, 13.5 mmol, 5.0 eq) and MeOH (6 mL). The resulting mixture was stirred for additional 4 hours at 25° C. The reaction was quenched with water, and extracted with CH₂Cl₂ (5 mL). The combined organic layers were washed with brine (3×5 mL), dried over anhydrous Na₂SO₄. After filtration, the filtrate was concentrated under vacuum. The crude residue was purified by Prep-TLC (CH₂Cl₂/MeOH=10:1) to give (R)-3-(2-isopropoxyphenyl)-1-((R)-tetrahydro-2H-pyran-3-yl)piperazine as a yellow oil (90 mg, 10.8%) and (R)-3-(2-isopropoxyphenyl)-1-((S)-tetrahydro-2H-pyran-3-yl)piperazine as a yellow oil (84 mg, 10.1%). LC-MS (ESI, m/z) M+1: 305.

Synthesis of 4-(2-((R)-2-(2-isopropoxyphenyl)-4-((R)-tetrahydro-2H-pyran-3-yl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide (assumed): Into an 8 mL vial were added (3R)-3-(2-isopropoxyphenyl)-1-[(3R)-oxan-3-yl]piperazine (assumed) (7 mg, 0.1 mmol, 1.0 eq), 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-N-[(3R)-5-nitro-3-(oxan-4-yl)-3,4-dihydro-2H-1,4-benzoxazin-7-ylsulfonyl]-4-{2-oxo-7-azaspiro[3.5]nonan-7-yl}benzamide (18 mg, 0.1 mmol, 1.0 eq), NaBH₃CN (7 mg, 0.1 mmol, 5.0 eq), ZnCl₂ (15 mg, 0.1 mmol, 5.0 eq) and MeOH (1 mL). The resulting mixture was stirred for additional 4 hours at 70° C. The reaction was quenched with water, and extracted with CH₂Cl₂ (5 mL). The combined organic layers were washed with brine (3×5 mL), dried over anhydrous Na₂SO₄. After filtration, the filtrate was concentrated under vacuum. The crude residue was purified by Prep-TLC (CH₂Cl₂/MeOH=16:1) to give 4-(2-((R)-2-(2-isopropoxyphenyl)-4-((R)-tetrahydro-2H-pyran-3-yl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide (assumed) as a yellow solid (10 mg, 39.4%). LC-MS (ESI, m/z) M+1: 1103. ¹H NMR (300 MHz, Chloroform-d) δ 12.58 (s, 1H), 8.41 (s, 1H), 8.36 (s, 1H), 8.29 (d, J=2.2 Hz, 1H), 8.10 (t, J=4.6 Hz, 2H), 7.44 (d, J=2.2 Hz, 2H), 7.15 (s, 1H), 6.82-6.80 (m, 1H), 6.67 (s, 1H), 6.41 (s, 1H), 6.11 (d, J=3.8 Hz, 1H), 4.69 (s, 1H), 4.63-4.32 (m, 4H), 4.19-4.02 (m, 5H), 3.95-3.91 (m, 2H), 3.82-3.80 (m, 2H), 3.56-3.23 (m, 10H), 2.99-2.85 (m, 7H), 2.01-1.95 (s, 4H), 1.44-1.40 (m, 6H), 1.32-1.28 (m, 9H), 1.18-0.74 (m, 8H).

### Example 175 Preparation of 4-(2-((R)-2-(2-isopropoxyphenyl)-4-((S)-tetrahydro-2H-pyran-3-yl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide (assumed)

Synthesis of 4-(2-((R)-2-(2-isopropoxyphenyl)-4-((S)-tetrahydro-2H-pyran-3-yl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide (assumed): Into an 8 mL vial were added (3R)-3-(2-isopropoxyphenyl)-1-[(3S)-oxan-3-yl]piperazine (assumed) (6 mg, 0.1 mmol, 1.0 eq), 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-N-[(3R)-5-nitro-3-(oxan-4-yl)-3,4-dihydro-2H-1,4-benzoxazin-7-ylsulfonyl]-4-{2-oxo-7-azaspiro[3.5]nonan-7-yl}benzamide (17 mg, 0.1 mmol, 1.0 eq), NaBH₃CN (6 mg, 0.1 mmol, 5.0 eq), ZnCl₂ (14 mg, 0.1 mmol, 5.0 eq) and MeOH (1 mL). The resulting mixture was stirred for additional 4 hours at 70° C. The reaction was quenched with water, and extracted with CH₂Cl₂ (5 mL). The combined organic layers were washed with brine (3×5 mL), dried over anhydrous Na₂SO₄. After filtration, the filtrate was concentrated under vacuum. The crude residue was purified by Prep-TLC (CH₂Cl₂/MeOH=15:1) to give 4-(2-((R)-2-(2-isopropoxyphenyl)-4-((S)-tetrahydro-2H-pyran-3-yl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide (assumed) as a yellow solid (10 mg, 42.4%). LC-MS (ESI, m/z) M+1: 1103. ¹H NMR (300 MHz, Chloroform-d) δ 12.62 (s, 1H), 8.42-8.40 (m, 2H), 8.31 (d, J=2.1 Hz, 1H), 8.18-8.08 (m, 1H), 7.46 (d, J=2.1 Hz, 2H), 7.18 (s, 1H), 7.09 (d, J=3.3 Hz, 1H), 6.84 (d, J=10.3 Hz, 3H), 6.69 (s, 1H), 6.43 (s, 1H), 6.14 (s, 1H), 4.78-4.38 (m, 4H), 4.36-4.02 (m, 4H), 3.91-3.85 (m, 3H), 3.76-3.21 (m, 10H), 3.06-3.01 (m, 5H), 2.81-2.31 (m, 5H), 2.06-2.00 (m, 3H), 1.80-1.76 (m, 3H), 1.47-1.42 (m, 4H), 1.35-1.33 (m, 1H), 1.19-0.73 (m, 4H).

### Example 176 Preparation of 4-(2-((R)-2-(2-isopropoxyphenyl)-4-((tetrahydro-2H-pyran-4-yl)methyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide (assumed)

Synthesis of 4-(2-((R)-2-(2-isopropoxyphenyl)-4-((tetrahydro-2H-pyran-4-yl)methyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide (assumed): Into an 8 mL vial were added (R)-3-(2-isopropoxyphenyl)-1-((tetrahydro-2H-pyran-4-yl)methyl)piperazine (assumed) (7 mg, 0.1 mmol, 1.0 eq), 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-N-[(3R)-5-nitro-3-(oxan-4-yl)-3,4-dihydro-2H-1,4-benzoxazin-7-ylsulfonyl]-4-{2-oxo-7-azaspiro[3.5]nonan-7-yl}benzamide (17 mg, 0.1 mmol, 1.0 eq), NaBH₃CN (7 mg, 0.1 mmol, 5.0 eq), ZnCl₂ (14 mg, 0.1 mmol, 5.0 eq) and MeOH (1 mL). The resulting mixture was stirred for additional 4 hours at 70° C. The reaction was quenched with water, and extracted with CH₂Cl₂ (5 mL). The combined organic layers were washed with brine (3×5 mL), dried over anhydrous Na₂SO₄. After filtration, the filtrate was concentrated under vacuum. The crude residue was purified by Prep-TLC (CH₂Cl₂/MeOH 15:1) to give 4-(2-((R)-2-(2-isopropoxyphenyl)-4-((tetrahydro-2H-pyran-4-yl)methyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide (assumed) as a yellow solid (10 mg, 40.7%). LC-MS (ESI, m/z) M+1: 1117. ¹H NMR (300 MHz, Chloroform-d) δ 12.62 (s, 1H), 8.45 (s, 1H), 8.38 (s, 1H), 8.31 (d, J=2.2 Hz, 1H), 8.20-8.09 (m, 1H), 7.46 (d, J=2.2 Hz, 2H), 7.22-7.07 (m, 2H), 6.84-6.80 (m, 2H), 6.69 (s, 1H), 6.43 (s, 1H), 6.14 (s, 1H), 4.77-4.37 (m, 4H), 4.24-4.03 (m, 4H), 3.90-3.85 (m, 4H), 3.42-3.40 (m, 8H), 3.07 (s, 3H), 2.90-2.85 (m, 3H), 2.63 (s, 2H), 2.45-2.01 (m, 6H), 1.78-1.74 (m, 8H), 1.55-1.42 (m, 5H), 1.34-1.33 (m, 6H), 1.09 (s, 3H), 0.99-0.83 (m, 3H).

### Example 177 Preparation of 4-(2-((R)-4-((5,6-dimethoxypyridin-3-yl)methyl)-2-(2-isopropoxyphenyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide (assumed)

Synthesis of (R)-1-((5,6-dimethoxypyridin-3-yl)methyl)-3-(2-isopropoxyphenyl)piperazine (assumed): Into a 40 mL vial were added 5,6-dimethoxypyridine-3-carbaldehyde (175 mg, 1.1 mmol, 1.0 eq), (R)-2-(2-isopropoxyphenyl)piperazine (assumed)(299 mg, 1.3 mmol, 1.3 eq), NaBH(OAc)₃ (1109 mg, 5.2 mmol, 5.0 eq) and DCE (3 mL). The resulting mixture was stirred for additional 2 hours at 25° C. The reaction was quenched by the addition of water (2 mL) and extracted with CH₂Cl₂ (3×10 mL). The combined organic layers were washed with brine (5 mL), dried over anhydrous Na₂SO₄. After filtration, the filtrate was concentrated under vacuum. The crude residue was purified by Prep-TLC (CH₂Cl₂/MeOH=10:1) to give (R)-1-((5,6-dimethoxypyridin-3-yl)methyl)-3-(2-isopropoxyphenyl)piperazine (assumed) as a light yellow oil (143 mg, 36.7%). LC-MS (ESI, m/z) M+1: 372.

Synthesis of 4-(2-((R)-4-((5,6-dimethoxypyridin-3-yl)methyl)-2-(2-isopropoxyphenyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide (assumed): Into an 8 mL vial were added (3R)-1-[(5,6-dimethoxypyridin-3-yl)methyl]-3-(2-isopropoxyphenyl)piperazine (assumed) (12 mg, 0.1 mmol, 1.0 eq), 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-N-[(3R)-5-nitro-3-(oxan-4-yl)-3,4-dihydro-2H-1,4-benzoxazin-7-ylsulfonyl]-4-{2-oxo-7-azaspiro[3.5]nonan-7-yl}benzamide (27 mg, 0.1 mmol, 1.0 eq), NaBH₃CN (10 mg, 0.2 mmol, 5.0 eq), ZnCl₂ (23 mg, 0.1 mmol, 5.0 eq) and MeOH (1 mL). The resulting mixture was stirred for additional 4 hours at 70° C. The reaction was quenched by the addition of water (5 mL), and extracted with CH₂Cl₂ (3×5 mL). The combined organic layers were washed with brine (5 mL), dried over anhydrous Na₂SO₄. After filtration, the filtrate was concentrated under vacuum. The crude residue was purified by Prep-TLC (CH₂Cl₂/MeOH 15:1) to give 4-{2-[(2R)-4-[(5,6-dimethoxypyridin-3-yl)methyl]-2-(2-isopropoxyphenyl)piperazin-1-yl]-7-azaspiro[3.5]nonan-7-yl}-2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-N-[(3R)-5-nitro-3-(oxan-4-yl)-3,4-dihydro-2H-1,4-benzoxazin-7-ylsulfonyl]benzamide (assumed) as a yellow solid (10 mg, 25.1%). LC-MS (ESI, m/z) M+1: 1170. ¹H NMR (300 MHz, Chloroform-d) δ 12.40 (s, 1H), 8.45 (s, 1H), 8.32 (d, J=2.2 Hz, 1H), 8.13-8.12 (m, 1H), 7.78-7.63 (m, 2H), 7.51-7.38 (m, 2H), 7.13 (s, 1H), 7.05 (d, J=7.5 Hz, 1H), 6.98-6.85 (m, 2H), 6.77 (s, 1H), 6.42 (s, 1H), 6.16 (s, 1H), 5.51-5.48 (m, 1H), 4.64-4.63 (m, 1H), 4.58-4.43 (m, 2H), 4.25-4.22 (m, 2H), 4.15-4.05 (m, 3H), 4.02-4.00 (m, 3H), 3.92-3.89 (m, 4H), 3.66-3.62 (m, 2H), 3.59-3.48 (m, 2H), 3.40-3.36 (m, 4H), 3.32 (s, 1H), 3.08-3.02 (m, 4H), 2.25-2.20 (m, 4H), 2.05-2.00 (m, 2H), 1.84-1.63 (m, 7H), 1.50-1.45 (m, 6H), 1.39 (s, 2H), 1.35-1.26 (m, 6H), 1.09-1.05 (m, 2H), 0.89-0.85 (m, 1H).

### Example 178 Preparation of N—(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)-4-(2-((R)-4-((R)-tetrahydro-2H-pyran-3-yl)-2-(o-tolyl)piperazin-1-yl)-7-azaspiro[3.5]nonan-7-yl)benzamide (assumed)

Synthesis of (R)-1-((R)-tetrahydro-2H-pyran-3-yl)-3-(o-tolyl)piperazine (assumed) and (R)-1-((S)-tetrahydro-2H-pyran-3-yl)-3-(o-tolyl)piperazine (assumed): Into a 40 mL vial were added oxan-3-one (284 mg, 2.8 mmol, 1.0 eq), (2R)-2-(2-methylphenyl)piperazine (500 mg, 2.8 mmol, 1.0 eq), NaBH₃CN (891.29 mg, 14.185 mmol, 5.0 eq), ZnCl₂ (1932 mg, 14.1 mmol, 5.0 eq) and MeOH (5 mL). The resulting mixture was stirred for additional 4 hours at 25° C. The reaction was quenched by the addition of water (5 mL), and extracted with EtOAc (3×5 mL). The combined organic layers were washed with brine (5 mL), dried over anhydrous Na₂SO₄. After filtration, the filtrate was concentrated under vacuum. The crude residue was purified by Prep-TLC (ethyl acetate/petroleum ether=1:5) to give (R)-1-((R)-tetrahydro-2H-pyran-3-yl)-3-(o-tolyl)piperazine (assumed) as a yellow solid (90 mg, 12.1%) and (R)-1-((S)-tetrahydro-2H-pyran-3-yl)-3-(o-tolyl)piperazine (assumed) as a yellow solid (88 mg, 11.9%). LC-MS (ESI, m/z) M+1: 261.

Synthesis of N—(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)-4-(2-((R)-4-((R)-tetrahydro-2H-pyran-3-yl)-2-(o-tolyl)piperazin-1-yl)-7-azaspiro[3.5]nonan-7-yl)benzamide (assumed): Into an 8 mL vial were added (R)-1-((R)-tetrahydro-2H-pyran-3-yl)-3-(o-tolyl)piperazine (assumed) (9 mg, 0.1 mmol, 1.0 eq), 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-N-[(3R)-5-nitro-3-(oxan-4-yl)-3,4-dihydro-2H-1,4-benzoxazin-7-ylsulfonyl]-4-{2-oxo-7-azaspiro[3.5]nonan-7-yl}benzamide (27 mg, 0.1 mmol, 1.0 equiv), NaBH₃CN (10 mg, 0.2 mmol, 5.0 eq), ZnCl₂ (23 mg, 0.2 mmol, 5.0 eq) and MeOH (1 mL). The resulting mixture was stirred for additional 4 hours at 70° C. The reaction was quenched by the addition of water (5 mL) and extracted with CH₂Cl₂ (3×5 mL). The combined organic layers were washed with brine (3 mL), dried over anhydrous Na₂SO₄. After filtration, the filtrate was concentrated under vacuum. The crude residue was purified by Prep-TLC (CH₂Cl₂/MeOH=15:1) to give N—(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)-4-(2-((R)-4-((R)-tetrahydro-2H-pyran-3-yl)-2-(o-tolyl)piperazin-1-yl)-7-azaspiro[3.5]nonan-7-yl)benzamide (assumed) as a yellow solid (10 mg, 27.6%). LC-MS (ESI, m/z) M+1: 1059. ¹H NMR (300 MHz, Chloroform-d) δ 12.58 (s, 1H), 8.41 (s, 1H), 8.36 (s, 1H), 8.29 (d, J=2.2 Hz, 1H), 8.10 (t, J=4.6 Hz, 2H), 7.44 (d, J=2.2 Hz, 2H), 7.15 (s, 1H), 7.10-7.03 (m, 1H), 6.82 (d, J=10.1 Hz, 3H), 6.67 (s, 1H), 6.41 (s, 1H), 6.11 (d, J=3.8 Hz, 1H), 4.69 (s, 1H), 4.63-4.32 (m, 4H), 4.19-4.02 (m, 5H), 3.95-3.91 (m, 2H), 3.82-3.81 (m, 2H), 3.56-3.23 (m, 10H), 2.99-2.95 (m, 7H), 2.01-1.98 (m, 3H), 1.44-1.40 (m, 6H), 1.32-1.29 (m, 9H), 1.18-1.14 (m, 2H).

### Example 179 Preparation of N—(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)-4-(2-((R)-4-((S)-tetrahydro-2H-pyran-3-yl)-2-(o-tolyl)piperazin-1-yl)-7-azaspiro[3.5]nonan-7-yl)benzamide (assumed)

Synthesis of N—(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)-4-(2-((R)-4-((S)-tetrahydro-2H-pyran-3-yl)-2-(o-tolyl)piperazin-1-yl)-7-azaspiro[3.5]nonan-7-yl)benzamide (assumed): Into an 8 mL vial were added (R)-1-((S)-tetrahydro-2H-pyran-3-yl)-3-(o-tolyl)piperazine (assumed) (8 mg, 0.1 mmol, 1.0 eq), 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-N-[(3R)-5-nitro-3-(oxan-4-yl)-3,4-dihydro-2H-1,4-benzoxazin-7-ylsulfonyl]-4-{2-oxo-7-azaspiro[3.5]nonan-7-yl}benzamide (27 mg, 0.1 mmol, 1.0 eq), NaBH₃CN (11 mg, 0.2 mmol, 5.0 eq), ZnCl₂ (23 mg, 0.2 mmol, 5.0 eq) and MeOH (1 mL). The resulting mixture was stirred for additional 4 hours at 70° C. The reaction was quenched with water, and extracted with CH₂Cl₂ (3×5 mL). The combined organic layers were washed with brine (5 mL), dried over anhydrous Na₂SO₄. After filtration, the filtrate was concentrated under vacuum. The crude residue was purified by Prep-TLC (CH₂Cl₂/MeOH=15:1) to give N—(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)-4-(2-((R)-4-((S)-tetrahydro-2H-pyran-3-yl)-2-(o-tolyl)piperazin-1-yl)-7-azaspiro[3.5]nonan-7-yl)benzamide (assumed) as a yellow solid (10 mg, 27.5%). LC-MS (ESI, m/z) M+1: 1059. ¹H NMR (300 MHz, Chloroform-d) δ 12.62 (s, 1H), 8.42 (d, J=14.4 Hz, 2H), 8.31 (d, J=2.1 Hz, 1H), 8.18-8.08 (m, 1H), 7.46 (d, J=2.1 Hz, 2H), 7.18 (s, 1H), 7.09 (d, J=3.3 Hz, 1H), 6.84 (d, J=10.3 Hz, 3H), 6.69 (s, 1H), 6.43 (s, 1H), 6.14 (s, 1H), 4.77-4.38 (m, 4H), 4.36-4.02 (m, 4H), 3.91-3.85 (m, 5H), 3.56-3.18 (m, 8H), 3.06-2.95 (m, 5H), 2.81-2.31 (m, 5H), 2.06-2.00 (m, 5H), 1.47-1.44 (m, 5H), 1.35-1.30 (m, 6H), 1.19-0.73 (m, 5H).

### Example 180 Preparation of 4-{2-[(2R)-4-[(3,4-difluorophenyl)methyl]-2-(2-isopropylphenyl)piperazin-1-yl]-7-azaspiro[3.5]nonan-7-yl}-2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-N-[(3R)-5-nitro-3-(oxan-4-yl)-3,4-dihydro-2H-1,4-benzoxazin-7-ylsulfonyl]benzamide

Synthesis of 4-{2-[(2R)-4-[(3,4-difluorophenyl)methyl]-2-(2-isopropylphenyl)piperazin-1-yl]-7-azaspiro[3.5]nonan-7-yl}-2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-N-[(3R)-5-nitro-3-(oxan-4-yl)-3,4-dihydro-2H-1,4-benzoxazin-7-ylsulfonyl]benzamide: Into an 8-mL sealed tube, were placed 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-N-[(3R)-5-nitro-3-(oxan-4-yl)-3,4-dihydro-2H-1,4-benzoxazin-7-ylsulfonyl]-4-{2-oxo-7-azaspiro[3.5]nonan-7-yl}benzamide (30 mg, 0.04 mmol, 1.0 eq), (3R)-1-[(3,4-difluorophenyl)methyl]-3-(2-isopropylphenyl)piperazine (18 mg, 0.06 mmol, 1.5 eq), ZnCl₂ (15 mg, 0.1 mmol, 3.0 eq), NaBH₃CN (7 mg, 0.1 mmol, 3.0 eq), MeOH (0.3 mL). The resulting solution was stirred for 3.5 hours at 70° C. The resulting mixture was then quenched by the addition of water (30 mL), and extracted with dichloromethane (2×30 mL). The combined organic layers was washed with brine (2×30 mL), dried over anhydrous Na₂SO₄. After filtration, the filtrate was concentrated under vacuum. The crude residue was applied onto a silica gel column and eluted with dichloromethane/methanol=15:1 to give 4-{2-[(2R)-4-[(3,4-difluorophenyl)methyl]-2-(2-isopropylphenyl)piperazin-1-yl]-7-azaspiro[3.5]nonan-7-yl}-2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-N-[(3R)-5-nitro-3-(oxan-4-yl)-3,4-dihydro-2H-1,4-benzoxazin-7-ylsulfonyl]benzamide as a yellow solid (2.8 mg, 6.7%). LC-MS (ESI, m/z) M+1: 1129. ¹H NMR (400 MHz, Methanol-d₄) δ 8.16-7.98 (m, 1H), 7.89-7.81 (m, 1H), 7.64-7.41 (m, 1H), 7.38-7.05 (m, 7H), 7.05-6.98 (m, 1H), 6.97-6.89 (m, 1H), 6.73-6.64 (m, 1H), 6.60-6.46 (m, 1H), 6.04-5.92 (m, 1H), 4.34-4.22 (m, 4H), 4.18-3.94 (m, 4H), 3.94-3.62 (m, 1H), 3.61-3.38 (m, 7H), 3.22-2.90 (m, 6H), 2.67-2.57 (m, 1H), 2.52-2.13 (m, 3H), 2.11-2.01 (m, 1H), 1.93-1.81 (m, 2H), 1.80-1.64 (m, 3H), 1.63-1.37 (m, 9H), 1.32-1.25 (m, 6H), 1.18-1.05 (m, 1H), 0.98-0.80 (m, 1H).

### Example 181 Preparation of 4-(2-((R)-4-(3,4-difluorobenzyl)-2-(2-isopropoxyphenyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide (assumed)

Synthesis of 4-(2-((R)-4-(3,4-difluorobenzyl)-2-(2-isopropoxyphenyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide (assumed): Into an 8-mL sealed-tube, were placed (3R)-1-[(3,4-difluorophenyl)methyl]-3-(2-isopropoxyphenyl)piperazine (15 mg, 0.04 mmol, 1.0 eq), 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-N-[(3R)-5-nitro-3-(oxan-4-yl)-3,4-dihydro-2H-1,4-benzoxazin-7-ylsulfonyl]-4-{2-oxo-7-azaspiro[3.5]nonan-7-yl}benzamide (35 mg, 0.04 mmol, 1.0 eq), NaBH₃CN (8 mg, 0.12 mmol, 3.0 eq), ZnCl₂ (18 mg, 0.12 mmol, 3.0 eq) and MeOH (1 mL). The resulting solution was stirred for 16 hours at 70° C. The resulting mixture was then quenched by the addition of water (5 mL), and extracted with dichloromethane/methanol (10:1, 2×10 mL). The combined organic layers was washed with brine (2×10 mL), dried over anhydrous Na₂SO₄. After filtration, the filtrate was concentrated under vacuum. The residue was purified by Prep-TLC (dichloromethane/methanol=15:1) to afford 4-(2-((R)-4-(3,4-difluorobenzyl)-2-(2-isopropoxyphenyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide (assumed) as a light yellow solid (15 mg, 30.5%). LC-MS (ESI, m/z) M+1: 1145. ¹H NMR (300 MHz, DMSO-d₆) δ 12.75 (s, 1H), 11.14 (s, 1H), 8.85 (s, 1H), 7.96 (d, J=2.2 Hz, 1H), 7.75-7.73 (m, 1H), 7.65 (s, 1H), 7.37-7.34 (m, 4H), 7.14-7.12 (m, 2H), 7.07-7.05 (m, 2H), 6.97-6.93 (m, 2H), 6.79 (s, 1H), 6.43 (s, 1H), 6.01 (s, 1H), 5.34-5.31 (m, 1H), 4.67-4.65 (m, 1H), 4.56-4.54 (m, 1H), 4.30-4.28 (m, 1H), 4.22 (s, 1H), 4.05-4.01 (m, 3H), 3.87-3.85 (m, 3H), 3.78-3.75 (m, 3H), 3.62-3.40 (m, 6H), 3.08-3.05 (m, 7H), 2.18-2.16 (m, 1H), 1.99-1.97 (m, 2H), 1.75-1.73 (m, 2H), 1.60-1.58 (m, 4H), 1.24-1.17 (m, 10H), 0.84-0.82 (m, 1H).

### Example 182 Preparation of 4-(2-((R)-2-(2-isopropoxyphenyl)-4-(tetrahydro-2H-pyran-4-yl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide (assumed)

Synthesis of (R)-3-(2-isopropoxyphenyl)-1-(tetrahydro-2H-pyran-4-yl)piperazine (assumed): Into a 40 mL vial were added tetrahydro-4H-pyran-4-one (136 mg, 1.3 mmol, 1.0 eq), (2R)-2-(2-isopropoxyphenyl)piperazine (assumed) (300 mg, 1.3 mmol, 1.0 eq), NaBH₃CN (426 mg, 6.8 mmol, 5.0 eq), ZnCl₂ (925 mg, 6.8 mmol, 5.0 eq) and MeOH (3 mL). The resulting mixture was stirred for 4 hours at 25° C. The reaction was quenched by the addition of water (10 mL), and extracted with CH₂Cl₂ (2×20 mL). The combined organic layers were washed with brine (20 mL), dried over anhydrous Na₂SO₄. After filtration, the filtrate was concentrated under vacuum. The residue was purified by Prep-TLC (dichloromethane/methanol=12:1) to give (R)-3-(2-isopropoxyphenyl)-1-(tetrahydro-2H-pyran-4-yl)piperazine (assumed) as a yellow oil (133 mg, 32.1%). LC-MS (ESI, m/z) M+1: 305.

Synthesis of 4-(2-((R)-2-(2-isopropoxyphenyl)-4-(tetrahydro-2H-pyran-4-yl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide (assumed): Into a 2-mL sealed-tube, were placed (3R)-3-(2-isopropoxyphenyl)-1-(oxan-4-yl)piperazine (7 mg, 0.03 mmol, 1.0 eq), 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-N-[(3R)-5-nitro-3-(oxan-4-yl)-3,4-dihydro-2H-1,4-benzoxazin-7-ylsulfonyl]-4-{2-oxo-7-azaspiro[3.5]nonan-7-yl}benzamide (20 mg, 0.03 mmol, 1.0 eq), NaBH₃CN (5 mg, 0.08 mmol, 3.0 eq), ZnCl₂ (10 mg, 0.08 mmol, 3.0 eq) and MeOH (0.5 mL). The resulting solution was stirred for 16 hours at 70° C. The resulting mixture was then quenched by the addition of water (5 mL), and extracted with dichloromethane/methanol (10:1, 2×10 mL). The combined organic layers was washed with brine (2×10 mL), dried over anhydrous Na₂SO₄. After filtration, the filtrate was concentrated under vacuum. The residue was purified by Prep-TLC (dichloromethane/methanol=15:1) to afford 4-(2-((R)-2-(2-isopropoxyphenyl)-4-(tetrahydro-2H-pyran-4-yl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide (assumed) as a light yellow solid (13 mg, 48.0%). LC-MS (ESI, m/z) M+1: 1103. ¹H NMR (400 MHz, DMSO-d₆) δ 12.73 (s, 1H), 11.10 (s, 1H), 8.00-7.95 (m, 1H), 7.83-7.54 (m, 1H), 7.39 (s, 1H), 7.20-7.15 (m, 2H), 7.08-7.05 (m, 2H), 7.01-6.68 (m, 4H), 6.46-6.42 (m, 1H), 5.99-5.93 (m, 1H), 4.62-4.58 (m, 2H), 4.29-4.20 (m, 3H), 4.03-3.95 (m, 3H), 3.83-3.60 (m, 9H), 3.55-3.40 (m, 5H), 3.12-2.76 (m, 8H), 2.01-1.90 (m, 2H), 1.76-1.70 (m, 4H), 1.61-1.55 (m, 6H), 1.31-1.26 (m, 11H), 0.85-0.80 (m, 2H).

### Example 183 Preparation of N—(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)-4-(2-((R)-4-(tetrahydro-2H-pyran-4-yl)-2-(o-tolyl)piperazin-1-yl)-7-azaspiro[3.5]nonan-7-yl)benzamide

Synthesis of (3R)-3-(2-methylphenyl)-1-(oxan-4-yl)piperazine: Into a 20-mL sealed-tube, were placed (2R)-2-(2-methylphenyl)piperazine (150 mg, 0.8 mmol, 1.0 eq), 4H-pyran-4-one (77 mg, 0.8 mmol, 0.9 eq), MeOH (3 mL), ZnCl₂ (348 mg, 2.5 mmol, 3.0 eq), NaBH₃CN (160 mg, 2.5 mmol, 3.0 eq). The resulting solution was stirred for 16 hours at 70° C. The resulting mixture was then quenched by the addition of water (30 mL), and extracted with dichloromethane/methanol (20:1, 2×40 mL). The combined organic layers was washed with brine (2×40 mL), dried over anhydrous Na₂SO₄. After filtration, the filtrate was concentrated under vacuum. The crude residue was purified onto a silica gel column and eluted with dichloromethane/methanol=10:1 to give (3R)-3-(2-methylphenyl)-1-(oxan-4-yl)piperazine as a light yellow oil (127 mg, 57.3%). LC-MS (ESI, m/z) M+1: 261.

Synthesis of N—(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)-4-(2-((R)-4-(tetrahydro-2H-pyran-4-yl)-2-(o-tolyl)piperazin-1-yl)-7-azaspiro[3.5]nonan-7-yl)benzamide: Into an 8-mL sealed-tube, were placed 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-N-[(3R)-5-nitro-3-(oxan-4-yl)-3,4-dihydro-2H-1,4-benzoxazin-7-ylsulfonyl]-4-{2-oxo-7-azaspiro[3.5]nonan-7-yl}benzamide (30 mg, 0.04 mmol, 1.0 eq), (3R)-3-(2-methylphenyl)-1-(oxan-4-yl)piperazine (14 mg, 0.05 mmol, 1.5 eq), MeOH (1 mL), NaBH₃CN (7 mg, 0.1 mmol, 3.0 eq), ZnCl₂ (15 mg, 0.1 mmol, 3.0 eq). The resulting solution was stirred for 3 hours at 70° C. The resulting mixture was then quenched by the addition of water (20 mL), and extracted with dichloromethane/methanol (10:1, 2×30 mL). The combined organic layers was washed with brine (2×20 mL), dried over anhydrous Na₂SO₄. After filtration, the filtrate was concentrated under vacuum. The residue was purified by Prep-TLC (dichloromethane/methanol=20:1) to give N—(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)-4-(2-((R)-4-(tetrahydro-2H-pyran-4-yl)-2-(o-tolyl)piperazin-1-yl)-7-azaspiro[3.5]nonan-7-yl)benzamide as a yellow solid (10 mg, 25.6%). LC-MS (ESI, m/z) M+1: 1059. ¹H NMR (400 MHz, Methanol-d₄) δ 8.10-8.02 (m, 1H), 7.85 (d, J=8.8 Hz, 1H), 7.47-7.40 (m, 1H), 7.23-7.14 (m, 4H), 7.04-6.94 (m, 2H), 6.76-6.69 (m, 1H), 6.50 (s, 1H), 6.08-6.99 (m, 1H), 4.32-4.21 (m, 2H), 4.14-4.10 (m, 1H), 4.02-3.90 (m, 6H), 3.76-3.64 (m, 1H), 3.52-3.38 (m, 6H), 3.17-3.10 (m, 4H), 3.04-2.99 (m, 1H), 2.93-2.82 (m, 2H), 2.62-2.49 (m, 3H), 2.40-2.33 (m, 3H), 1.90-1.82 (m, 4H), 1.79-1.66 (m, 6H), 1.60-1.39 (m, 6H), 1.36-1.25 (m, 4H), 1.20-1.11 (m, 3H).

### Example 184 Preparation of 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-4-{2-[(2R)-2-(2-methylphenyl)-4-(oxan-4-ylmethyl)piperazin-1-yl]-7-azaspiro[3.5]nonan-7-yl}-N-[(3R)-5-nitro-3-(oxan-4-yl)-3,4-dihydro-2H-1,4-benzoxazin-7-ylsulfonyl]benzamide

Synthesis of (3R)-3-(2-methylphenyl)-1-(oxan-4-ylmethyl)piperazine: Into a 20-mL sealed-tube, were placed oxane-4-carbaldehyde (87 mg, 0.8 mmol, 0.9 eq), (2R)-2-(2-methylphenyl)piperazine (150 mg, 0.9 mmol, 1.0 eq), MeOH (2 mL), ZnCl₂ (348 mg, 2.5 mmol, 3.0 eq), NaBH₃CN (160 mg, 2.5 mmol, 3.0 eq). The resulting solution was stirred for 16 hours at 25° C. The resulting mixture was then quenched by the addition of water (30 mL), and extracted with dichloromethane/methanol (20:1, 2×40 mL). The combined organic layers was washed with brine (2×40 mL), dried over anhydrous Na₂SO₄. After filtration, the filtrate was concentrated under vacuum. The crude residue was purified onto a silica gel column and eluted with dichloromethane/methanol=10:1 to give (3R)-3-(2-methylphenyl)-1-(oxan-4-ylmethyl)piperazine as a light yellow oil (125 mg, 53.5%). LC-MS (ESI, m/z) M+1: 275.

Synthesis of 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-4-{2-[(2R)-2-(2-methylphenyl)-4-(oxan-4-ylmethyl)piperazin-1-yl]-7-azaspiro[3.5]nonan-7-yl}-N-[(3R)-5-nitro-3-(oxan-4-yl)-3,4-dihydro-2H-1,4-benzoxazin-7-ylsulfonyl]benzamide: Into an 8-mL sealed-tube, were placed 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-N-[(3R)-5-nitro-3-(oxan-4-yl)-3,4-dihydro-2H-1,4-benzoxazin-7-ylsulfonyl]-4-{2-oxo-7-azaspiro[3.5]nonan-7-yl}benzamide (20 mg, 0.03 mmol, 1.0 eq), (3R)-3-(2-methylphenyl)-1-(oxan-4-ylmethyl)piperazine (7 mg, 0.03 mmol, 1.0 eq), MeOH (1 mL), ZnCl₂ (10 mg, 0.07 mmol, 3.0 eq), NaBH₃CN (2 mg, 0.07 mmol, 3.0 eq). The resulting solution was stirred for 3 hours at 70° C. The resulting mixture was then quenched by the addition of water (20 mL), and extracted with dichloromethane/methanol (10:1, 2×30 mL). The combined organic layers was washed with brine (2×20 mL), dried over anhydrous Na₂SO₄. After filtration, the filtrate was concentrated under vacuum. The residue was purified by Prep-TLC (dichloromethane/methanol=20:1) to give 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-4-{2-[(2R)-2-(2-methylphenyl)-4-(oxan-4-ylmethyl)piperazin-1-yl]-7-azaspiro[3.5]nonan-7-yl}-N-[(3R)-5-nitro-3-(oxan-4-yl)-3,4-dihydro-2H-1,4-benzoxazin-7-ylsulfonyl]benzamide as a yellow solid (8 mg, 30.4%). LC-MS (ESI, m/z) M+1: 1073. ¹H NMR (400 MHz, Methanol-d₄) δ 8.02 (d, J=2.2 Hz, 1H), 7.84 (d, J=9.0 Hz, 1H), 7.45-7.40 (m, 1H), 7.23-7.15 (m, 4H), 7.05-6.91 (m, 2H), 6.72-6.63 (m, 1H), 6.50 (s, 1H), 6.03-5.99 (m, 1H), 4.31-4.22 (m, 3H), 4.15-4.11 (m, 1H), 4.02-3.90 (m, 5H), 3.82-3.65 (m, 2H), 3.52-3.37 (m, 7H), 3.174-3.04 (m, 7H), 2.77-2.73 (m, 1H), 2.53-2.25 (m, 9H), 1.92-1.82 (m, 3H), 1.77-1.59 (m, 5H), 1.57-1.40 (m, 5H), 1.36-1.11 (m, 6H).

### Example 185 Preparation of 4-{2-[(2S)-4-[(3,4-difluorophenyl)methyl]-2-(2-isopropoxyphenyl)piperazin-1-yl]-7-azaspiro[3.5]nonan-7-yl}-2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-N-{3-nitro-4-[(oxan-4-ylmethyl)amino]benzenesulfonyl}benzamide (assumed)

Synthesis of 4-{2-[(2S)-4-[(3,4-difluorophenyl)methyl]-2-(2-isopropoxyphenyl)piperazin-1-yl]-7-azaspiro[3.5]nonan-7-yl}-2-[(3R,8S)-15-{[2-(trimethylsilyl)ethoxy]methyl}-2,5-dioxa-9,15,17-triazatetracyclo[8.7.0.0\hat{ }{3,8}.0\hat{ }{12,16}]heptadeca-1(10),11,13,16-tetraen-9-yl]benzoate (assumed): Into an 8-mL sealed-tube, were placed (3S)-1-[(3,4-difluorophenyl)methyl]-3-(2-isopropoxyphenyl)piperazine (assumed) (60 mg, 0.2 mmol, 1.0 eq), methyl 4-{2-oxo-7-azaspiro[3.5]nonan-7-yl}-2-[(3R,8S)-15-{[2-(trimethylsilyl)ethoxy]methyl}-2,5-dioxa-9,15,17-triazatetracyclo[8.7.0.0\hat{ }{3,8}.0\hat{ }{12,16}]heptadeca-1(10),11,13,16-tetraen-9-yl]benzoate (110 mg, 0.2 mmol, 1.0 eq), MeOH (2 mL), ZnCl₂ (71 mg, 0.5 mmol, 3.0 eq) and NaBH₃CN (33 mg, 0.5 mmol, 3.0 eq). The resulting solution was stirred for 4 hours at 70° C. The resulting mixture was then quenched by the addition of water (10 mL), and extracted with dichloromethane/methanol (10:1, 2×20 mL). The combined organic layers was washed with brine (2×20 mL), dried over anhydrous Na₂SO₄. After filtration, the filtrate was concentrated under vacuum. The residue was purified by Prep-TLC (dichloromethane/methanol=15:1) to give methyl 4-{2-[(2S)-4-[(3,4-difluorophenyl)methyl]-2-(2-isopropoxyphenyl)piperazin-1-yl]-7-azaspiro[3.5]nonan-7-yl}-2-[(3R,8S)-15-{[2-(trimethylsilyl)ethoxy]methyl}-2,5-dioxa-9,15,17-triazatetracyclo[8.7.0.0\hat{ }{3,8}.0\hat{ }{12,16}]heptadeca-1(10),11,13,16-tetraen-9-yl]benzoate (assumed) as a light yellow solid (100 mg, 59.9%). LC-MS (ESI, m/z) M+1: 964.

Synthesis of 4-{2-[(2S)-4-[(3,4-difluorophenyl)methyl]-2-(2-isopropoxyphenyl)piperazin-1-yl]-7-azaspiro[3.5]nonan-7-yl}-2-[(3R,8S)-15-{[2-(trimethylsilyl)ethoxy]methyl}-2,5-dioxa-9,15,17-triazatetracyclo[8.7.0.0\hat{ }{3,8}.0\hat{ }{12,16}]heptadeca-1(10),11,13,16-tetraen-9-yl]benzoic acid (assumed): Into an 8 mL sealed tube were added methyl 4-{2-[(2S)-4-[(3,4-difluorophenyl)methyl]-2-(2-isopropoxyphenyl)piperazin-1-yl]-7-azaspiro[3.5]nonan-7-yl}-2-[(3R,8S)-15-{[2-(trimethylsilyl)ethoxy]methyl}-2,5-dioxa-9,15,17-triazatetracyclo[8.7.0.0\hat{ }{3,8}.0\hat{ }{12,16}]heptadeca-1(10),11,13,16-tetraen-9-yl]benzoate (assumed) (100 mg, 0.1 mmol, 1.0 eq), dioxane (1 mL), MeOH (1 mL) and NaOH (200 uL, 4 M) at 25° C. The resulting mixture was stirred for 16 hours at 50° C. The mixture was acidified to pH=5 with HCl (2 M). The resulting mixture was extracted with CH₂Cl₂ (2×20 mL). The combined organic layers were washed with brine (20 mL), dried over anhydrous Na₂SO₄. After filtration, the filtrate was concentrated under vacuum. The residue was purified by Prep-TLC (dichloromethane/methanol=10:1) to afford 4-{2-[(2S)-4-[(3,4-difluorophenyl)methyl]-2-(2-isopropoxyphenyl)piperazin-1-yl]-7-azaspiro[3.5]nonan-7-yl}-2-[(3R,8S)-15-{[2-(trimethylsilyl)ethoxy]methyl}-2,5-dioxa-9,15,17-triazatetracyclo[8.7.0.0\hat{ }{3,8}.0\hat{ }{12,16}]heptadeca-1(10),11,13,16-tetraen-9-yl]benzoic acid (assumed) as a colorless oil (85 mg, 86.3%). LC-MS (ESI, m/z) M+1: 949.

Synthesis of 4-{2-[(2S)-4-[(3,4-difluorophenyl)methyl]-2-(2-isopropoxyphenyl)piperazin-1-yl]-7-azaspiro[3.5]nonan-7-yl}-N-{3-nitro-4-[(oxan-4-ylmethyl)amino]benzenesulfonyl}-2-[(3R,8S)-15-{[2-(trimethylsilyl)ethoxy]methyl}-2,5-dioxa-9,15,17-triazatetracyclo[8.7.0.0\hat{ }{3,8}.0\hat{ }{12,16}]heptadeca-1(10),11,13,16-tetraen-9-yl]benzamide (assumed): Into an 8 mL sealed tube were added 4-{2-[(2S)-4-[(3,4-difluorophenyl)methyl]-2-(2-isopropoxyphenyl)piperazin-1-yl]-7-azaspiro[3.5]nonan-7-yl}-2-[(3R,8S)-15-{[2-(trimethylsilyl)ethoxy]methyl}-2,5-dioxa-9,15,17-triazatetracyclo[8.7.0.0\hat{ }{3,8}.0\hat{ }{12,16}]heptadeca-1(10),11,13,16-tetraen-9-yl]benzoic acid (assumed) (80 mg, 0.1 mmol, 1.0 eq), 3-nitro-4-[(oxan-4-ylmethyl)amino]benzenesulfonamide (27 mg, 0.1 mmol, 1.0 eq), EDCl (32 mg, 0.2 mmol, 2.0 eq), DMAP (41 mg, 0.3 mmol, 4.0 eq) and DCM (2 mL) at 25° C. The resulting mixture was stirred for 4 hours at 40° C. The reaction was quenched by the addition of water (2 mL), and extracted with CH₂Cl₂ (2×10 mL). The combined organic layers were washed with brine (20 mL), dried over anhydrous Na₂SO₄. After filtration, the filtrate was concentrated under reduced pressure. The residue was purified by Prep-TLC (dichloromethane/methanol=10:1) to afford 4-{2-[(2S)-4-[(3,4-difluorophenyl)methyl]-2-(2-isopropoxyphenyl)piperazin-1-yl]-7-azaspiro[3.5]nonan-7-yl}-N-{3-nitro-4-[(oxan-4-ylmethyl)amino]benzenesulfonyl}-2-[(3R,8S)-15-{[2-(trimethylsilyl)ethoxy]methyl}-2,5-dioxa-9,15,17-triazatetracyclo[8.7.0.0\hat{ }{3,8}.0\hat{ }{12,16}]heptadeca-1(10),11,13,16-tetraen-9-yl]benzamide (assumed) as a yellow solid (60 mg, 57.1%). LC-MS (ESI, m/z) M+1: 1246.

Synthesis of 4-{2-[(2S)-4-[(3,4-difluorophenyl)methyl]-2-(2-isopropoxyphenyl)piperazin-1-yl]-7-azaspiro[3.5]nonan-7-yl}-2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-N-{3-nitro-4-[(oxan-4-ylmethyl)amino]benzenesulfonyl}benzamide (assumed): Into a 40 mL vial, were placed 4-{2-[(2S)-4-[(3,4-difluorophenyl)methyl]-2-(2-isopropoxyphenyl)piperazin-1-yl]-7-azaspiro[3.5]nonan-7-yl}-N-{3-nitro-4-[(oxan-4-ylmethyl)amino]benzenesulfonyl}-2-[(3R,8S)-15-{[2-(trimethylsilyl)ethoxy]methyl}-2,5-dioxa-9,15,17-triazatetracyclo[8.7.0.0\hat{ }{3,8}.0\hat{ }{12,16}]heptadeca-1(10),11,13,16-tetraen-9-yl]benzamide (assumed) (50 mg, 0.04 mmol, 1.0 eq), ethylenediamine (48 mg, 0.8 mmol, 20 eq), TBAF in THE (1 M, 1 mL). The resulting mixture was stirred for 6 hours at 60° C. The resulting mixture was diluted with EtOAc (10 mL), and washed with water (10×20 mL). The organic layer was dried over anhydrous Na₂SO₄. After filtration, the filtrate was concentrated under vacuum. The residue was purified by Prep-TLC (dichloromethane/methanol=10:1) to afford 4-{2-[(2S)-4-[(3,4-difluorophenyl)methyl]-2-(2-isopropoxyphenyl)piperazin-1-yl]-7-azaspiro[3.5]nonan-7-yl}-2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-N-{3-nitro-4-[(oxan-4-ylmethyl)amino]benzenesulfonyl}benzamide (assumed) as a yellow solid (24 mg, 53.6%). LC-MS (ESI, m/z) M+1: 1116. ¹H NMR (300 MHz, DMSO-d₆) δ 12.74 (s, 1H), 11.22 (s, 1H), 8.60 (s, 1H), 8.37 (s, 1H), 7.38-7.33 (m, 4H), 7.20-7.12 (m, 3H), 6.92 (s, 1H), 6.88-6.82 (m, 4H), 6.43 (s, 1H), 6.05 (s, 1H), 4.54 (s, 1H), 4.21 (s, 1H), 4.05 (t, J=7.6 Hz, 1H), 3.92-3.89 (m, 2H), 3.88-3.80 (m, 3H), 3.73 (s, 1H), 3.50-3.42 (m, 3H), 3.15-3.08 (m, 13H), 2.92-2.87 (m, 2H), 2.81 (s, 1H), 2.20-2.17 (m, 2H), 1.92-1.89 (m, 3H), 1.72 (s, 1H), 1.65-1.60 (m, 2H), 1.49 (s, 1H), 1.35-1.30 (m, 8H), 1.27-1.23 (m, 2H), 1.16-1.10 (m, 2H).

### Example 186 Preparation of 4-{2-[(2R)-4-[(3,4-difluorophenyl)methyl]-2-(2-isopropoxyphenyl)piperazin-1-yl]-7-azaspiro[3.5]nonan-7-yl}-2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-N-{3-nitro-4-[(oxan-4-ylmethyl)amino]benzenesulfonyl}benzamide (assumed)

Synthesis of 4-{2-[(2R)-4-[(3,4-difluorophenyl)methyl]-2-(2-isopropoxyphenyl)piperazin-1-yl]-7-azaspiro[3.5]nonan-7-yl}-2-[(3R,8S)-15-{[2-(trimethylsilyl)ethoxy]methyl}-2,5-dioxa-9,15,17-triazatetracyclo[8.7.0.0\hat{ }{3,8}.0\hat{ }{12,16}]heptadeca-1(10),11,13,16-tetraen-9-yl]benzoate (assumed): Into an 8-mL sealed-tube, were placed (3R)-1-[(3,4-difluorophenyl)methyl]-3-(2-isopropoxyphenyl)piperazine (assumed) (60 mg, 0.2 mmol, 1.0 eq), methyl 4-{2-oxo-7-azaspiro[3.5]nonan-7-yl}-2-[(3R,8S)-15-{[2-(trimethylsilyl)ethoxy]methyl}-2,5-dioxa-9,15,17-triazatetracyclo[8.7.0.0\hat{ }{3,8}.0\hat{ }{12,16}]heptadeca-1(10),11,13,16-tetraen-9-yl]benzoate (110 mg, 0.2 mmol, 1.0 eq), MeOH (2 mL), ZnCl₂ (71 mg, 0.5 mmol, 3.0 eq) and NaBH₃CN (33 mg, 0.5 mmol, 3.0 eq). The resulting solution was stirred for 4 hours at 70° C. The resulting mixture was then quenched by the addition of water (10 mL), and extracted with dichloromethane/methanol (10:1, 2×20 mL). The combined organic layers was washed with brine (2×20 mL) dried over anhydrous Na₂SO₄. After filtration, the filtrate was concentrated under vacuum. The residue was purified by Prep-TLC (dichloromethane/methanol=15:1) to give methyl 4-{2-[(2R)-4-[(3,4-difluorophenyl)methyl]-2-(2-isopropoxyphenyl)piperazin-1-yl]-7-azaspiro[3.5]nonan-7-yl}-2-[(3R,8S)-15-{[2-(trimethylsilyl)ethoxy]methyl}-2,5-dioxa-9,15,17-triazatetracyclo[8.7.0.0\hat{ }{3,8}.0\hat{ }{12,16}]heptadeca-1(10),11,13,16-tetraen-9-yl]benzoate (assumed) as a light yellow solid (100 mg, 59.9%). LC-MS (ESI, m/z) M+1: 964.

Synthesis of 4-{2-[(2R)-4-[(3,4-difluorophenyl)methyl]-2-(2-isopropoxyphenyl)piperazin-1-yl]-7-azaspiro[3.5]nonan-7-yl}-2-[(3R,8S)-15-{[2-(trimethylsilyl)ethoxy]methyl}-2,5-dioxa-9,15,17-triazatetracyclo[8.7.0.0\hat{ }{3,8}.0\hat{ }{12,16}]heptadeca-1(10),11,13,16-tetraen-9-yl]benzoic acid (assumed): Into an 8 mL sealed tube were added methyl 4-{2-[(2R)-4-[(3,4-difluorophenyl)methyl]-2-(2-isopropoxyphenyl)piperazin-1-yl]-7-azaspiro[3.5]nonan-7-yl}-2-[(3R,8S)-15-{[2-(trimethylsilyl)ethoxy]methyl}-2,5-dioxa-9,15,17-triazatetracyclo[8.7.0.0\hat{ }{3,8}.0\hat{ }{12,16}]heptadeca-1(10),11,13,16-tetraen-9-yl]benzoate (assumed) (100 mg, 0.1 mmol, 1.0 eq), dioxane (1 mL), MeOH (1 mL) and NaOH (200 uL, 4 M) at 25° C. The resulting mixture was stirred for 16 hours at 50° C. The mixture was acidified to pH=5 with HCl (2 M). The resulting mixture was extracted with CH₂Cl₂ (2×20 mL). The combined organic layers were washed with brine (20 mL), dried over anhydrous Na₂SO₄. After filtration, the filtrate was concentrated under vacuum. The residue was purified by Prep-TLC (dichloromethane/methanol=10:1) to afford 4-{2-[(2R)-4-[(3,4-difluorophenyl)methyl]-2-(2-isopropoxyphenyl)piperazin-1-yl]-7-azaspiro[3.5]nonan-7-yl}-2-[(3R,8S)-15-{[2-(trimethylsilyl)ethoxy]methyl}-2,5-dioxa-9,15,17-triazatetracyclo[8.7.0.0\hat{ }{3,8}.0\hat{ }{12,16}]heptadeca-1(10),11,13,16-tetraen-9-yl]benzoic acid (assumed) as a colorless oil (85 mg, 86.3%). LC-MS (ESI, m/z) M+1: 949.

Synthesis of 4-{2-[(2R)-4-[(3,4-difluorophenyl)methyl]-2-(2-isopropoxyphenyl)piperazin-1-yl]-7-azaspiro[3.5]nonan-7-yl}-N-{3-nitro-4-[(oxan-4-ylmethyl)amino]benzenesulfonyl}-2-[(3R,8S)-15-{[2-(trimethylsilyl)ethoxy]methyl}-2,5-dioxa-9,15,17-triazatetracyclo[8.7.0.0\hat{ }{3,8}.0\hat{ }{12,16}]heptadeca-1(10),11,13,16-tetraen-9-yl]benzamide (assumed): Into an 8 mL sealed tube were added 4-{2-[(2R)-4-[(3,4-difluorophenyl)methyl]-2-(2-isopropoxyphenyl)piperazin-1-yl]-7-azaspiro[3.5]nonan-7-yl}-2-[(3R,8S)-15-{[2-(trimethylsilyl)ethoxy]methyl}-2,5-dioxa-9,15,17-triazatetracyclo[8.7.0.0\hat{ }{3,8}.0\hat{ }{12,16}]heptadeca-1(10),11,13,16-tetraen-9-yl]benzoic acid (assumed) (80 mg, 0.1 mmol, 1.0 eq), 3-nitro-4-[(oxan-4-ylmethyl)amino]benzenesulfonamide (27 mg, 0.1 mmol, 1.0 eq), EDCl (32 mg, 0.2 mmol, 2.0 eq), DMAP (41 mg, 0.3 mmol, 4.0 eq) and DCM (2 mL) at 25° C. The resulting mixture was stirred for 4 hours at 40° C. The reaction was quenched by the addition of water (2 mL), and extracted with CH₂Cl₂ (2×10 mL). The combined organic layers were washed with brine (20 mL), dried over anhydrous Na₂SO₄. After filtration, the filtrate was concentrated under reduced pressure. The residue was purified by Prep-TLC (dichloromethane/methanol=10:1) to afford 4-{2-[(2R)-4-[(3,4-difluorophenyl)methyl]-2-(2-isopropoxyphenyl)piperazin-1-yl]-7-azaspiro[3.5]nonan-7-yl}-N-{3-nitro-4-[(oxan-4-ylmethyl)amino]benzenesulfonyl}-2-[(3R,8S)-15-{[2-(trimethylsilyl)ethoxy]methyl}-2,5-dioxa-9,15,17-triazatetracyclo[8.7.0.0\hat{ }{3,8}.0\hat{ }{12,16}]heptadeca-1(10),11,13,16-tetraen-9-yl]benzamide (assumed) as a yellow solid (60 mg, 57.1%). LC-MS (ESI, m/z) M+1: 1246.

Synthesis of 4-{2-[(2R)-4-[(3,4-difluorophenyl)methyl]-2-(2-isopropoxyphenyl)piperazin-1-yl]-7-azaspiro[3.5]nonan-7-yl}-2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-N-{3-nitro-4-[(oxan-4-ylmethyl)amino]benzenesulfonyl}benzamide (assumed): Into a 40 mL vial, were placed 4-{2-[(2R)-4-[(3,4-difluorophenyl)methyl]-2-(2-isopropoxyphenyl)piperazin-1-yl]-7-azaspiro[3.5]nonan-7-yl}-N-{3-nitro-4-[(oxan-4-ylmethyl)amino]benzenesulfonyl}-2-[(3R,8S)-15-{[2-(trimethylsilyl)ethoxy]methyl}-2,5-dioxa-9,15,17-triazatetracyclo[8.7.0.0\hat{ }{3,8}.0\hat{ }{12,16}]heptadeca-1(10),11,13,16-tetraen-9-yl]benzamide (assumed) (50 mg, 0.04 mmol, 1.0 eq), ethylenediamine (48 mg, 0.8 mmol, 20 eq), TBAF in THE (1 M, 1 mL). The resulting mixture was stirred for 6 hours at 60° C. The resulting mixture was diluted with EtOAc (10 mL), and washed with water (10×20 mL), dried over anhydrous Na₂SO₄. After filtration, the filtrate was concentrated under vacuum. The residue was purified by Prep-TLC (dichloromethane/methanol=10:1) to afford 4-{2-[(2R)-4-[(3,4-difluorophenyl)methyl]-2-(2-isopropoxyphenyl)piperazin-1-yl]-7-azaspiro[3.5]nonan-7-yl}-2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-N-{3-nitro-4-[(oxan-4-ylmethyl)amino]benzenesulfonyl}benzamide (assumed) as a yellow solid (24 mg, 53.6%). LC-MS (ESI, m/z) M+1: 1116. ¹H NMR (300 MHz, DMSO-d₆) δ 12.75 (s, 1H), 11.23 (s, 1H), 8.61 (s, 1H), 8.37 (s, 1H), 7.38-7.33 (m, 4H), 7.20-7.12 (m, 3H), 6.9-6.78 (m, 5H), 6.43 (s, 1H), 6.05 (s, 1H), 4.55 (s, 1H), 4.22 (s, 1H), 4.06 (s, 1H), 3.92-3.80 (m, 5H), 3.74-3.71 (m, 2H), 3.50-3.42 (m, 3H), 3.17-3.06 (m, 12H), 2.92-2.87 (m, 2H), 2.81 (s, 1H), 2.20-2.17 (m, 2H), 1.92-1.89 (m, 3H), 1.72 (s, 1H), 1.65-1.60 (m, 2H), 1.49 (s, 1H), 1.37-1.32 (m, 8H), 1.28-1.23 (m, 2H), 1.17-1.10 (m, 2H).

### Example 187 Preparation of 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-4-{2-[(2R)-4-({5-methoxy-6- [(3S)-3-methylmorpholin-4-yl]pyridin-3-yl}methyl)-2-(2-methylphenyl)piperazin-1-yl]-7-azaspiro[3.5]nonan-7-yl}-N-[(3R)-5-nitro-3-(oxan-4-yl)-3,4-dihydro-2H-1,4-benzoxazin-7-ylsulfonyl]benzamide

Synthesis of (S)-4-(3-methoxy-5-(((R)-3-(o-tolyl)piperazin-1-yl)methyl)pyridin-2-yl)-3-methylmorpholine: Into an 8 mL sealed tube were added 5-methoxy-6-[(3S)-3-methylmorpholin-4-yl]pyridine-3-carbaldehyde (150 mg, 0.6 mmol, 0.8 eq), (2R)-2-(2-methylphenyl)piperazine (140 mg, 0.8 mmol, 1.0 eq), NaBH₃CN (150 mg, 2.4 mmol, 3.0 eq), MeOH (3 mL) and ZnCl₂ (325 mg, 2.4 mmol, 3.0 eq) at 25° C. The resulting mixture was stirred for 16 hours at 25° C. The reaction was quenched by the addition of water (2 mL), and extracted with CH₂Cl₂ (2×10 mL). The combined organic layers were washed with brine (20 mL), dried over anhydrous Na₂SO₄. After filtration, the filtrate was concentrated under vacuum. The residue was purified by Prep-TLC (dichloromethane/methanol=12:1) to give (S)-4-(3-methoxy-5-(((R)-3-(o-tolyl)piperazin-1-yl)methyl)pyridin-2-yl)-3-methylmorpholine (assumed) as a colorless oil (80 mg, 25.4%). LC-MS (ES, m/z) M+1: 397.

Synthesis of 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-4-{2-[(2R)-4-({5-methoxy-6-[(3S)-3-methylmorpholin-4-yl]pyridin-3-yl}methyl)-2-(2-methylphenyl)piperazin-1-yl]-7-azaspiro[3.5]nonan-7-yl}-N-[(3R)-5-nitro-3-(oxan-4-yl)-3,4-dihydro-2H-1,4-benzoxazin-7-ylsulfonyl]benzamide: Into an 8 mL vial were added (3S)-4-(3-methoxy-5-{[(3R)-3-(2-methylphenyl)piperazin-1-yl]methyl}pyridin-2-yl)-3-methylmorpholine (20 mg, 0.05 mmol, 1.0 eq), 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-N-[(3R)-5-nitro-3-(oxan-4-yl)-3,4-dihydro-2H-1,4-benzoxazin-7-ylsulfonyl]-4-{2-oxo-7-azaspiro[3.5]nonan-7-yl}benzamide (41 mg, 0.05 mmol, 1.0 eq), NaBH₃CN (21 mg, 0.3 mmol, 5.0 eq), ZnCl₂ (46 mg, 0.3 mmol, 5.0 eq) and MeOH (1 mL). The resulting mixture was stirred for overnight at 70° C. The reaction was quenched by the addition of water (3 mL) and extracted with CH₂Cl₂ (3×5 mL). The combined organic layers were washed with brine (5 mL), dried over anhydrous Na₂SO₄. After filtration, the filtrate was concentrated under vacuum. The residue was purified by Prep-TLC (CH₂Cl₂/MeOH=15:1) to give 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-4-{2-[(2R)-4-({5-methoxy-6- [(3S)-3-methylmorpholin-4-yl]pyridin-3-yl}methyl)-2-(2-methylphenyl)piperazin-1-yl]-7-azaspiro[3.5]nonan-7-yl}-N-[(3R)-5-nitro-3-(oxan-4-yl)-3,4-dihydro-2H-1,4-benzoxazin-7-ylsulfonyl]benzamide as a yellow solid (12 mg, 19.9%). LC-MS (ES, m/z) M+1: 1195. ¹H NMR (300 MHz, Chloroform-d) δ 12.62 (s, 1H), 8.43 (d, J=12.2 Hz, 2H), 8.31 (d, J=2.2 Hz, 1H), 8.12 (d, J=9.1 Hz, 1H), 7.75 (s, 1H), 7.46 (d, J=2.2 Hz, 1H), 7.09-7.06 (m, 4H), 6.86 (s, 1H), 6.68 (s, 1H), 6.44 (s, 1H), 6.13 (s, 1H), 4.65-4.31 (m, 3H), 4.31-4.00 (m, 5H), 4.00-3.65 (m, 6H), 3.44-3.41 (m, 8H), 3.34-2.85 (m, 7H), 2.37-2.36 (m, 5H), 1.78-1.62 (m, 15H), 1.51-0.98 (m, 10H).

### Example 188 Preparation of 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-4-{2-[(2R)-4-({5-methoxy-6- [(3R)-3-methylmorpholin-4-yl]pyridin-3-yl}methyl)-2-(2-methylphenyl)piperazin-1-yl]-7-azaspiro[3.5]nonan-7-yl}-N-[(3R)-5-nitro-3-(oxan-4-yl)-3,4-dihydro-2H-1,4-benzoxazin-7-ylsulfonyl]benzamide

Synthesis of (3R)-4-(3-methoxy-5-{[(3R)-3-(2-methylphenyl)piperazin-1-yl]methyl}pyridin-2-yl)-3-methylmorpholine: Into an 8 mL vial were added (2R)-2-(2-methylphenyl)piperazine (70 mg, 0.4 mmol, 1.0 eq,), 5-methoxy-6-[(3R)-3-methylmorpholin-4-yl]pyridine-3-carbaldehyde (93 mg, 0.4 mmol, 1.0 eq), NaBH₃CN (124 mg, 2.0 mmol, 5.0 eq), ZnCl₂ (270 mg, 2.0 mmol, 5.0 eq) and MeOH (2 mL). The resulting mixture was stirred for 4 hours at 25° C. The residue was purified by Prep-TLC (CH₂Cl₂/MeOH=10:1) to give (3R)-4-(3-methoxy-5-{[(3R)-3-(2-methylphenyl)piperazin-1-yl]methyl}pyridin-2-yl)-3-methylmorpholine as a colorless solid (40 mg, 25.4%). LC-MS (ES, m/z) M+1: 397.

Synthesis of 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-4-{2-[(2R)-4-({5-methoxy-6-[(3R)-3-methylmorpholin-4-yl]pyridin-3-yl}methyl)-2-(2-methylphenyl)piperazin-1-yl]-7-azaspiro[3.5]nonan-7-yl}-N-[(3R)-5-nitro-3-(oxan-4-yl)-3,4-dihydro-2H-1,4-benzoxazin-7-ylsulfonyl]benzamide: Into an 8 mL vial were added (3R)-4-(3-methoxy-5-{[(3R)-3-(2-methylphenyl)piperazin-1-yl]methyl}pyridin-2-yl)-3-methylmorpholine (20 mg, 0.05 mmol, 1.0 eq), 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-N-[(3R)-5-nitro-3-(oxan-4-yl)-3,4-dihydro-2H-1,4-benzoxazin-7-ylsulfonyl]-4-{2-oxo-7-azaspiro[3.5]nonan-7-yl}benzamide (41 mg, 0.05 mmol, 1.0 eq), NaBH₃CN (21 mg, 0.3 mmol, 5.0 eq), ZnCl₂ (46 mg, 0.3 mmol, 5.0 eq) and MeOH (1 mL). The resulting mixture was stirred for overnight at 70° C. The reaction was quenched by the addition of water (3 mL) and extracted with CH₂Cl₂ (3×5 mL). The combined organic layers were washed with brine (5 mL), dried over anhydrous Na₂SO₄. After filtration, the filtrate was concentrated under vacuum. The residue was purified by Prep-TLC (CH₂Cl₂/MeOH=15:1) to give 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-4-{2-[(2R)-4-({5-methoxy-6- [(3R)-3-methylmorpholin-4-yl]pyridin-3-yl}methyl)-2-(2-methylphenyl)piperazin-1-yl]-7-azaspiro[3.5]nonan-7-yl}-N-[(3R)-5-nitro-3-(oxan-4-yl)-3,4-dihydro-2H-1,4-benzoxazin-7-ylsulfonyl]benzamide as a yellow solid (8 mg, 13.2%). LC-MS (ES, m/z) M+1: 1195. ¹H NMR (300 MHz, Chloroform-d) δ 12.63 (s, 1H), 8.42 (d, J=19.9 Hz, 2H), 8.31 (d, J=2.2 Hz, 1H), 8.13 (d, J=9.1 Hz, 1H), 7.76 (s, 1H), 7.46 (d, J=2.2 Hz, 2H), 7.23-7.04 (m, 4H), 6.85 (d, J=10.0 Hz, 1H), 6.69 (s, 1H), 6.44 (s, 1H), 6.14 (dd, J=3.5, 1.9 Hz, 1H), 4.63-4.37 (m, 2H), 4.26-4.03 (m, 5H), 3.88-3.81 (m, 7H), 3.72-3.60 (m, 2H), 3.60-3.33 (m, 8H), 3.25-3.21 (m, 2H), 3.16-2.81 (m, 7H), 2.67-2.56 (m, 2H), 2.31-2.19 (m, 6H), 1.78-1.72 (m, 5H), 1.43-1.36 (m, 6H), 1.31-1.27 (m, 5H).

### Example 189 Preparation of 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-4-{2-[(2R)-4-({5-fluoro-6-[(3S)-3-methylmorpholin-4-yl]pyridin-3-yl}methyl)-2-(2-methylphenyl)piperazin-1-yl]-7-azaspiro[3.5]nonan-7-yl}-N-[(3R)-5-nitro-3-(oxan-4-yl)-3,4-dihydro-2H-1,4-benzoxazin-7-ylsulfonyl]benzamide

Synthesis of (3S)-4-(5-bromo-3-fluoropyridin-2-yl)-3-methylmorpholine: Into a 100 mL round-bottom flask, were placed 5-bromo-2,3-difluoropyridine (5.0 g, 25.8 mmol, 1.0 eq), (3S)-3-methylmorpholine (2.6 g, 25.8 mmol, 1.0 eq), K₂CO₃ (10.7 g, 77.3 mmol, 3.0 eq), DMSO (50 mL). The resulting solution was stirred for overnight at 110° C. in an oil bath. The resulting mixture was then quenched by the addition of water (100 mL), and then extracted with ethyl acetate (2×100 mL). The combined organic layers was washed with water (2×100 mL) and brine (2×100 mL), dried over anhydrous Na₂SO₄. After filtration, the filtrate was concentrated under vacuum. The crude residue was applied onto a silica gel column and eluted with ethyl acetate/petroleum ether=1:3 to give (3S)-4-(5-bromo-3-fluoropyridin-2-yl)-3-methylmorpholine as a light yellow oil (2.3 g, 32.4%). LC-MS (ES, m/z) M+1: 275/277.

Synthesis of 5-fluoro-6-[(3S)-3-methylmorpholin-4-yl]pyridine-3-carbaldehyde: Into a 100 mL 3-necked round-bottom flask under nitrogen atmosphere, were placed (3S)-4-(5-bromo-3-fluoropyridin-2-yl)-3-methylmorpholine (1.0 g, 3.6 mmol, 1.0 eq), THE (10 mL). This was followed by the addition of n-BuLi (350 mg, 5.5 mmol, 1.5 eq) at −78° C. The resulting mixture was stirred for 1 hour at −78° C. To the above mixture was added DMF (370 mg, 5.0 mmol, 1.4 eq) at −78° C. The resulting mixture was stirred for additional 1 hour at −78° C. The resulting mixture was then quenched by the addition of water (50 mL), and then extracted with ethyl acetate (2×50 mL). The combined organic layers was washed with brine (2×50 mL), dried over anhydrous Na₂SO₄. After filtration, the filtrate was concentrated under vacuum. The crude residue was purified onto a silica gel column and eluted with ethyl acetate/petroleum ether=1:3 to give 5-fluoro-6-[(3S)-3-methylmorpholin-4-yl]pyridine-3-carbaldehyde as a light yellow oil (150 mg, 18.4%). LC-MS (ES, m/z) M+1: 225.

Synthesis of (3S)-4-(3-fluoro-5-{[(3R)-3-(2-methylphenyl)piperazin-1-yl]methyl}pyridin-2-yl)-3-methylmorpholine: Into an 8-mL sealed tube, were placed 5-fluoro-6-[(3S)-3-methylmorpholin-4-yl]pyridine-3-carbaldehyde (70 mg, 0.3 mmol, 1.0 eq), (2R)-2-(2-methylphenyl)piperazine (55 mg, 0.3 mmol, 1.0 eq), NaBH(OAc)₃ (198 mg, 0.9 mmol, 3.0 eq), DCE (1 mL). The resulting solution was stirred for 2 hours at 25° C. The resulting mixture was then quenched by the addition of water (30 mL), and then extracted with dichloromethane (2×30 mL). The combined organic layers was washed with brine (2×30 mL), dried over anhydrous Na₂SO₄. After filtration, the filtrate was concentrated under vacuum. The crude residue was applied onto a silica gel column and eluted with dichloromethane/methanol=10:1 to give (3S)-4-(3-fluoro-5-{[(3R)-3-(2-methylphenyl)piperazin-1-yl]methyl}pyridin-2-yl)-3-methylmorpholine as a light yellow solid (60 mg, 50.0%). LC-MS (ES, m/z) M+1: 385.

Synthesis of 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-4-{2-[(2R)-4-({5-fluoro-6-[(3S)-3-methylmorpholin-4-yl]pyridin-3-yl}methyl)-2-(2-methylphenyl)piperazin-1-yl]-7-azaspiro[3.5]nonan-7-yl}-N-[(3R)-5-nitro-3-(oxan-4-yl)-3,4-dihydro-2H-1,4-benzoxazin-7-ylsulfonyl]benzamide: Into an 8-mL sealed tube, were placed 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-N-[(3R)-5-nitro-3-(oxan-4-yl)-3,4-dihydro-2H-1,4-benzoxazin-7-ylsulfonyl]-4-{2-oxo-7-azaspiro[3.5]nonan-7-yl}benzamide (20 mg, 0.03 mmol, 1.0 eq), (3S)-4-(3-fluoro-5-{[(3R)-3-(2-methylphenyl)piperazin-1-yl]methyl}pyridin-2-yl)-3-methylmorpholine (11 mg, 0.03 mmol, 1.2 eq), ZnCl₂ (10 mg, 0.08 mmol, 3.0 eq), NaBH₃CN (5 mg, 0.07 mmol, 3.0 eq), MeOH (0.5 mL). The resulting solution was stirred for 4 hours at 70° C. in an oil bath. The resulting mixture was then quenched by the addition of water (30 mL), and then extracted with dichloromethane (2×30 mL). The combined organic layers was washed with brine (2×30 mL), dried over anhydrous Na₂SO₄. After filtration, the filtrate was concentrated under vacuum. The crude residue was applied onto a silica gel column and eluted with dichloromethane/methanol=15:1 to give 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-4-{2-[(2R)-4-({5-fluoro-6-[(3S)-3-methylmorpholin-4-yl]pyridin-3-yl}methyl)-2-(2-methylphenyl)piperazin-1-yl]-7-azaspiro[3.5]nonan-7-yl}-N-[(3R)-5-nitro-3-(oxan-4-yl)-3,4-dihydro-2H-1,4-benzoxazin-7-ylsulfonyl]benzamide as a yellow solid (10 mg, 34.4%). LC-MS (ES, m/z) M+1: 1183. ¹H NMR (400 MHz, Methanol-d₄) δ 8.05-8.00 (m, 1H), 7.94-7.91 (m, 1H), 7.89-7.84 (m, 1H), 7.49-7.37 (m, 2H), 7.24-7.19 (m, 1H), 7.14-7.12 (m, 3H), 7.06-7.01 (m, 1H), 6.99-6.89 (m, 1H), 6.72-6.67 (m, 1H), 6.51-6.47 (m, 1H), 6.02-5.95 (d, J=3.4 Hz, 1H), 4.34-4.27 (m, 2H), 4.27-4.20 (m, 1H), 4.16-4.09 (m, 2H), 4.04-3.97 (m, 2H), 3.95-3.88 (m, 2H), 3.87-3.79 (m, 1H), 3.73-3.63 (m, 1H), 3.61-3.47 (m, 9H), 3.20-3.09 (m, 6H), 2.99-2.95 (m, 1H), 2.74-2.66 (m, 1H), 2.41-2.34 (m, 4H), 1.92-1.87 (m, 2H), 1.79-1.65 (m, 4H), 1.63-1.44 (m, 3H), 1.43-1.33 (m, 6H), 1.22-1.16 (m, 6H), 1.02-0.87 (m, 2H).

### Example 190 Preparation of 4-(2-((R)-4-((5-isopropoxy-6-((S)-3-methylmorpholino)pyridin-3-yl)methyl)-2-(o-tolyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide (assumed)

Synthesis of (S)-4-(3-isopropoxypyridin-2-yl)-3-methylmorpholine: A solution of 2-chloro-3-isopropoxypyridine (5.0 g, 29.1 mmol, 1.0 equiv), (3S)-3-methylmorpholine (4.4 g, 43.7 mmol, 1.5 eq), t-BuOK (9.1 g, 87.4 mmol, 3.0 eq) and CPHOS PD G3 (0.2 g, 0.2 mmol, 0.1 eq) in 1,4-dioxane was stirred for 16 hours at 110° C. under nitrogen atmosphere. The reaction was quenched with water at 25° C. The resulting mixture was filtered, the filter cake was washed with EtOAc (3×10 mL). The filtrate was concentrated under vacuum. The resulting mixture was diluted with EtOAc (10 mL), and then washed with brine (3×10 mL), dried over anhydrous Na₂SO₄. After filtration, the filtrate was concentrated under vacuum. The crude residue was purified by silica gel column chromatography, eluted with ethyl acetate/petroleum ether=1:5 to give (S)-4-(3-isopropoxypyridin-2-yl)-3-methylmorpholine as a light yellow oil (3.1 g, 45.0%). LC-MS (ES, m/z) M+1: 237.

Synthesis of (S)-4-(3-isopropoxypyridin-2-yl)-3-methylmorpholine: Into a 250 mL round-bottom flask were added (S)-4-(3-isopropoxypyridin-2-yl)-3-methylmorpholine (3.0 g, 12.6 mmol, 1.0 eq), NBS (4.5 g, 25.3 mmol, 2 eq) and DMF (30 mL). The resulting mixture was stirred for additional 2 hours at 80° C. The reaction was quenched with water at 25° C. The resulting mixture was extracted with EtOAc (10 mL). The organic layer was washed with brine (3×10 mL), dried over anhydrous Na₂SO₄. After filtration, the filtrate was concentrated under vacuum. The crude residue was purified by silica gel column chromatography, eluted with ethyl acetate/petroleum ether=1:5 to give (S)-4-(5-bromo-3-isopropoxypyridin-2-yl)-3-methylmorpholine as a dark red oil (1.8 g, 44.8%). LC-MS (ES, m/z) M+1: 315/317. ¹H NMR (300 MHz, Chloroform-d) δ 7.92 (d, J=2.0 Hz, 1H), 7.15 (d, J=2.1 Hz, 1H), 4.67-4.45 (m, 1H), 4.22 (dt, J=6.6, 3.2 Hz, 1H), 3.98-3.60 (m, 4H), 3.51-3.30 (m, 2H), 1.37-1.35 (m, 6H), 1.18-1.15 (m, 3H).

Synthesis of (S)-5-isopropoxy-6-(3-methylmorpholino)nicotinaldehyde: A solution of (3S)-4-(5-bromo-3-isopropoxypyridin-2-yl)-3-methylmorpholine (1.2 g, 3.8 mmol, 1.0 eq) in THE (10 mL) was treated with n-BuLi (1 mL, 19.3 mmol, 5.0 eq) for 1 hour at −78° C. under nitrogen atmosphere, which was followed by the addition of DMF (1669 mg, 22.8 mmol, 6.0 eq) dropwise at −78° C. The resulting mixture was stirred for additional 1 hours at −78° C. The reaction was quenched by the addition of sat. NH₄Cl (aq.) (15 mL) at 0° C., and then extracted with EtOAc (3×10 mL). The combined organic layers were washed with brine (5 mL), dried over anhydrous Na₂SO₄. After filtration, the filtrate was concentrated under vacuum. The crude residue was purified by silica gel column chromatography, eluted with ethyl acetate/petroleum ether=1:5 to give (S)-5-isopropoxy-6-(3-methylmorpholino)nicotinaldehyde as a light yellow solid (586 mg, 58.3%). LC-MS (ES, m/z) M+1: 265. ¹H NMR (300 MHz, Chloroform-d) δ 9.83 (s, 1H), 8.22 (d, J=1.9 Hz, 1H), 7.28 (s, 1H), 4.75-4.57 (m, 1H), 4.22-4.15 (m, 1H), 3.96-3.91 (m, 1H), 3.74-3.72 (m, 3H), 3.50-3.46 (m, 1H), 1.42-1.38 (m, 7H), 1.37 (d, J=3.8 Hz, 3H).

Synthesis of (S)-4-(3-isopropoxy-5-(((R)-3-(o-tolyl)piperazin-1-yl)methyl)pyridin-2-yl)-3-methylmorpholine (assumed): Into a 40 mL vial were added 5-isopropoxy-6-[(3S)-3-methylmorpholin-4-yl]pyridine-3-carbaldehyde (120 mg, 0.4 mmol, 1.0 eq), (2R)-2-(2-methylphenyl)piperazine (80 mg, 0.4 mmol, 1.0 eq), NaBH(OAc)₃ (481.09 mg, 2.270 mmol, 5.0 eq) and DCE (2 mL). The resulting mixture was stirred for additional 3 hours at 25° C. The reaction was quenched with water, and then extracted with CH₂Cl₂ (5 mL). The combined organic layers were washed with brine (3×5 mL), dried over anhydrous Na₂SO₄. After filtration, the filtrate was concentrated under vacuum. The crude residue was purified by Prep-TLC (CH₂Cl₂/MeOH=10:1) to give (S)-4-(3-isopropoxy-5-(((R)-3-(o-tolyl)piperazin-1-yl)methyl)pyridin-2-yl)-3-methylmorpholine (assumed) as a yellow oil (100 mg, 51.8%). LC-MS (ES, m/z) M+1: 425.

Synthesis of 4-(2-((R)-4-((5-isopropoxy-6-((S)-3-methylmorpholino)pyridin-3-yl)methyl)-2-(o-tolyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide (assumed): Into an 8 mL vial were added (3S)-4-(3-isopropoxy-5-{[(3R)-3-(2-methylphenyl)piperazin-1-yl]methyl}pyridin-2-yl)-3-methylmorpholine (assumed) (10 mg, 0.1 mmol, 1.0 eq), 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-N-[(3R)-5-nitro-3-(oxan-4-yl)-3,4-dihydro-2H-1,4-benzoxazin-7-ylsulfonyl]-4-{2-oxo-7-azaspiro[3.5]nonan-7-yl}benzamide (19 mg, 0.1 mmol, 1.0 eq), NaBH₃CN (7 mg, 0.1 mmol, 5.0 eq), ZnCl₂ (16 mg, 0.1 mmol, 5.0 eq) and MeOH (2 mL). The resulting mixture was stirred for additional 4 hours at 70° C. The reaction was quenched with water, and then extracted with CH₂Cl₂ (5 mL). The combined organic layers were washed with brine (3×5 mL), dried over anhydrous Na₂SO₄. After filtration, the filtrate was concentrated under vacuum. The crude residue was purified by Prep-TLC (CH₂Cl₂/MeOH=15:1) to give 4-(2-((R)-4-((5-isopropoxy-6-((S)-3-methylmorpholino)pyridin-3-yl)methyl)-2-(o-tolyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide (assumed) as a yellow solid (10 mg, 34.3%). LC-MS (ES, m/z) M+1:1224. ¹H NMR (300 MHz, Chloroform-d) δ 12.32 (s, 1H), 10.11 (s, 1H), 8.45 (s, 1H), 8.32 (d, J=2.0 Hz, 1H), 8.13 (d, J=8.4 Hz, 1H), 7.97 (s, 1H), 7.83 (s, 1H), 7.44 (s, 1H), 7.33 (s, 2H), 7.15 (s, 1H), 6.87 (d, J=8.9 Hz, 1H), 6.80 (s, 1H), 6.43 (s, 1H), 6.17 (s, 1H), 5.13 (s, 1H), 4.61-4.53 (m, 12H), 4.44-4.40 (m, 8H), 4.08-4.00 (m, 3H), 3.72-3.68 (m, 11H), 3.40-3.36 (m, 3H), 3.06 (s, 2H), 2.41 (s, 3H), 2.20 (s, 2H), 2.08 (s, 1H), 1.78-1.72 (m, 2H), 1.67-1.60 (m, 3H), 1.41-1.38 (m, 9H), 1.01-0.93 (m, 4H).

### Example 191 Preparation of 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-4-{2-[(2R)-4-({2-methyl-7-[(3S)-3-methylmorpholin-4-yl]furo[2,3-c]pyridin-4-yl}methyl)-2-(2-methylphenyl)piperazin-1-yl]-7-azaspiro[3.5]nonan-7-yl}-N-[(3R)-5-nitro-3-(oxan-4-yl)-3,4-dihydro-2H-1,4-benzoxazin-7-ylsulfonyl]benzamide

Synthesis of (3S)-3-methyl-4-{2-methylfuro[2,3-c]pyridin-7-yl}morpholine: Into a 40 mL seal tube were added 7-chloro-2-methylfuro[2,3-c]pyridine (2.0 g, 11.9 mmol, 1.0 eq), (3S)-3-methylmorpholine (1.2 g, 11.9 mmol, 1.0 eq), t-BuOK (4.0 g, 35.8 mmol, 3.0 eq), dioxane (20 mL), CPhos Pd G3 (200 mg, 10%). The resulting mixture was stirred for overnight at 110° C. under nitrogen atmosphere. The precipitated solids were collected by filtration and washed with CH₂Cl₂ (3×20 mL). The resulting mixture was diluted with water (20 mL), and then extracted with CH₂Cl₂ (3×20 mL). The combined organic layers were washed with brine (3×20 mL), dried over anhydrous Na₂SO₄. After filtration, the filtrate was concentrated under vacuum. The crude residue was purified by silica gel column chromatography, eluted with dichloromethane/methanol (15:1) to give (3S)-3-methyl-4-{2-methylfuro[2,3-c]pyridin-7-yl}morpholine as a yellow oil (2.4 g, 86.6%). ¹H NMR (300 MHz, DMSO-d₆) δ 7.84 (d, J=5.3 Hz, 1H), 6.91 (d, J=5.3 Hz, 1H), 6.55 (q, J=1.1 Hz, 1H), 4.71-4.57 (m, 1H), 4.16-3.88 (m, 2H), 3.72 (t, J=2.1 Hz, 2H), 3.57 (td, J=11.6, 2.8 Hz, 1H), 3.40-3.23 (m, 1H), 2.45 (d, J=1.1 Hz, 3H), 1.24-1.11 (m, 3H).

Synthesis of (3S)-4-{4-bromo-2-methylfuro[2,3-c]pyridin-7-yl}-3-methylmorpholine: Into a 100 mL 3-necked round-bottom flask, were placed (3S)-3-methyl-4-{2-methylfuro[2,3-c]pyridin-7-yl}morpholine (2.0 g, 8.6 mmol, 1.0 eq), NBS (3.1 g, 17.2 mmol, 2.0 eq), DMF (20 mL). The resulting mixture was stirred for 12 hours at 80° C. in an oil bath. The resulting mixture was then quenched by the addition of water (200 mL), and then extracted with dichloromethane (2×200 mL). The combined organic layers was washed with brine (2×200 mL), dried over anhydrous Na₂SO₄. After filtration, the filtrate was concentrated under vacuum. The crude residue was purified onto a silica gel column and eluted with ethyl acetate/petroleum ether=1:2 to give (3S)-4-{4-bromo-2-methylfuro[2,3-c]pyridin-7-yl}-3-methylmorpholine as a brown oil (1.1 g, 41.1%). ¹H NMR (300 MHz, DMSO-d₆) δ 7.95 (s, 1H), 6.58 (q, J=1.0 Hz, 1H), 4.66-4.55 (m, 1H), 4.12-4.03 (m, 1H), 3.94 (ddd, J=11.3, 3.7, 1.4 Hz, 1H), 3.72 (d, J=2.3 Hz, 2H), 3.56 (td, J=11.6, 2.8 Hz, 1H), 3.33 (s, 4H), 1.19 (d, J=6.8 Hz, 3H).

Synthesis of 2-methyl-7-[(3S)-3-methylmorpholin-4-yl]furo[2,3-c]pyridine-4-carbaldehyde: Into a 100 mL 3-necked round-bottom flask under nitrogen atmosphere, were placed (3S)-4-{4-bromo-2-methylfuro[2,3-c]pyridin-7-yl}-3-methylmorpholine (1.0 g, 3.2 mmol, 1.0 eq), THE (10 mL). This was followed by the addition of n-BuLi (309 mg, 4.8 mmol, 1.5 eq) at −78° C. The resulting mixture was stirred for 30 minutes at −78° C. To the above mixture was added DMF (329 mg, 4.5 mmol, 1.4 eq) at −78° C. The resulting mixture was stirred for additional 30 minutes at −78° C. The resulting mixture was then quenched by the addition of water (200 mL), and then extracted with ethyl acetate (2×100 mL). The combined organic layers was washed with brine (2×100 mL). The mixture was dried over anhydrous Na₂SO₄. After filtration, the filtrate was concentrated under vacuum. The crude residue was purified onto a silica gel column and eluted with ethyl acetate/petroleum ether=1:1 to give 2-methyl-7-[(3S)-3-methylmorpholin-4-yl]furo[2,3-c]pyridine-4-carbaldehyde as a yellow solid (440 mg, 52.6%). ¹H NMR (400 MHz, DMSO-d₆) δ 9.89 (s, 1H), 8.41 (s, 1H), 7.08 (d, J=1.2 Hz, 1H), 4.99-4.90 (m, 1H), 4.56 (d, J=12.9 Hz, 1H), 4.04-3.94 (m, 1H), 3.81-3.67 (m, 2H), 3.62-3.42 (m, 2H), 2.52 (d, J=1.0 Hz, 3H), 1.32 (d, J=6.8 Hz, 3H).

Synthesis of (3S)-3-methyl-4-(2-methyl-4-{[(3R)-3-(2-methylphenyl)piperazin-1-yl]methyl}furo[2,3-c]pyridin-7-yl)morpholine: Into an 8-mL sealed tube purged and maintained with an inert atmosphere of nitrogen, were placed 2-methyl-7-[(3S)-3-methylmorpholin-4-yl]furo[2,3-c]pyridine-4-carbaldehyde (120 mg, 0.5 mmol, 1.0 eq), (2R)-2-(2-methylphenyl)piperazine (122 mg, 0.7 mmol, 1.5 eq), ZnCl₂ (188 mg, 1.4 mmol, 3.0 eq), NaBH₃CN (87 mg, 1.4 mmol, 3.0 eq), MeOH (1 mL). The resulting solution was stirred for 4 hours at 25° C. The resulting mixture was then quenched by the addition of water (30 mL). The resulting solution was extracted with dichloromethane (2×30 mL) and washed with brine (2×30 mL) and the organic layers combined. The mixture was dried over anhydrous Na₂SO₄. After filtration, the filtrate was concentrated under vacuum. The crude residue was applied onto a silica gel column and eluted with dichloromethane/methanol=10:1 to give (3S)-3-methyl-4-(2-methyl-4-{[(3R)-3-(2-methylphenyl)piperazin-1-yl]methyl}furo[2,3-c]pyridin-7-yl)morpholine as a yellow oil (130 mg, 67.1%). LC-MS (ES, m/z) M+1: 421.

Synthesis of 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-4-{2-[(2R)-4-({2-methyl-7-[(3S)-3-methylmorpholin-4-yl]furo[2,3-c]pyridin-4-yl}methyl)-2-(2-methylphenyl)piperazin-1-yl]-7-azaspiro[3.5]nonan-7-yl}-N-[(3R)-5-nitro-3-(oxan-4-yl)-3,4-dihydro-2H-1,4-benzoxazin-7-ylsulfonyl]benzamide: Into an 8-mL sealed tube, were placed 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-N-[(3R)-5-nitro-3-(oxan-4-yl)-3,4-dihydro-2H-1,4-benzoxazin-7-ylsulfonyl]-4-{2-oxo-7-azaspiro[3.5]nonan-7-yl}benzamide (20 mg, 0.03 mmol, 1.0 eq), (3S)-3-methyl-4-(2-methyl-4-{[(3R)-3-(2-methylphenyl)piperazin-1-yl]methyl}furo[2,3-c]pyridin-7-yl)morpholine (12 mg, 0.03 mmol, 1.2 eq), ZnCl₂ (10 mg, 0.08 mmol, 3.0 eq), NaBH₃CN (5 mg, 0.08 mmol, 3.0 eq), MeOH (0.5 mL). The resulting solution was stirred for 4 hours at 70° C. in an oil bath. The resulting mixture was then quenched by the addition of water (30 mL). The resulting solution was extracted with dichloromethane (2×30 mL) and washed with brine (2×30 mL) and the organic layers combined. The mixture was dried over anhydrous Na₂SO₄. After filtration, the filtrate was concentrated under vacuum. The crude residue was applied onto a silica gel column and eluted with dichloromethane/methanol=15:1 to give 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-4-{2-[(2R)-4-({2-methyl-7-[(3S)-3-methylmorpholin-4-yl]furo[2,3-c]pyridin-4-yl}methyl)-2-(2-methylphenyl)piperazin-1-yl]-7-azaspiro[3.5]nonan-7-yl}-N-[(3R)-5-nitro-3-(oxan-4-yl)-3,4-dihydro-2H-1,4-benzoxazin-7-ylsulfonyl]benzamide as a yellow solid (8 mg, 26.7%). LC-MS (ES, m/z) M+1: 1219. ¹H NMR (400 MHz, Methanol-d₄) δ 8.04-7.98 (m, 1H), 7.90-7.84 (m, 1H), 7.77-7.72 (m, 1H), 7.47-7.41 (m, 1H), 7.24-7.13 (m, 4H), 7.07-7.01 (m, 1H), 6.98-6.92 (m, 1H), 6.72-6.66 (m, 2H), 6.52-6.45 (m, 1H), 6.02-5.94 (m, 1H), 4.75-4.68 (m, 1H), 4.34-4.20 (m, 2H), 4.17-4.09 (m, 1H), 4.06-3.96 (m 4H), 3.95-3.81 (m, 1H), 3.80-3.61 (m, 2H), 3.52-3.39 (m, 4H), 3.21-2.98 (m, 6H), 2.82-2.73 (m, 1H), 2.52-2.42 (m, 4H), 2.38-2.33 (m, 2H), 1.98-1.85 (m, 3H), 1.82-1.65 (m, 4H), 1.63-1.37 (m, 6H), 1.35-1.28 (m, 8H), 1.27-1.23 (m, 6H), 1.19-1.03 (m, 1H), 0.93-0.83 (m, 2H).

### Example 192 Preparation of 4-{2-[(2R)-4-{[4,4-dimethyl-8-(morpholin-4-yl)-2H,3H-pyrano[2,3-c]pyridin-5-yl]methyl}-2-(2-methylphenyl)piperazin-1-yl]-7-azaspiro[3.5]nonan-7-yl}-2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-N-[(3R)-5-nitro-3-(oxan-4-yl)-3,4-dihydro-2H-1,4-benzoxazin-7-ylsulfonyl]benzamide

Synthesis of 4-{4,4-dimethyl-2H,3H-pyrano[2,3-c]pyridin-8-yl}morpholine: Into a 250 mL round-bottom flask, were placed 8-chloro-4,4-dimethyl-2H,3H-pyrano[2,3-c]pyridine (15.0 g, 75.9 mmol, 1.0 eq), morpholine (150 mL). The resulting solution was stirred for overnight at 130° C. The resulting mixture was then quenched by the addition of water (200 mL). The resulting solution was extracted with ethyl acetate (2×200 mL) and washed with brine (2×200 mL) and the organic layers combined. The mixture was dried over anhydrous Na₂SO₄. After filtration, the filtrate was concentrated under vacuum. The resulting mixture was concentrated under vacuum. The crude residue was applied onto a silica gel column and eluted with petroleum ether/ethyl acetate=3:1 to give 4-{4,4-dimethyl-2H,3H-pyrano[2,3-c]pyridin-8-yl}morpholine as a red oil (1.2 g, 6.4%). ¹H NMR (300 MHz, DMSO-d₆) δ 7.68 (d, J=5.2 Hz, 1H), 6.90 (d, J=5.2 Hz, 1H), 4.22-4.14 (m, 2H), 3.73-3.65 (m, 4H), 3.25-3.18 (m, 4H), 1.82-1.77 (m, 2H), 1.27 (s, 6H).

Synthesis of 4-{5-bromo-4,4-dimethyl-2H,3H-pyrano[2,3-c]pyridin-8-yl}morpholine: Into a 40-mL sealed tube, were placed 4-{4,4-dimethyl-2H,3H-pyrano[2,3-c]pyridin-8-yl}morpholine (1.3 g, 5.2 mmol, 1.0 eq), NBS (1.9 g, 10.5 mmol, 2.0 eq), DMF (15 mL). The resulting mixture was stirred for 3 hours at 80° C. The reaction mixture was quenched by the addition of water (500 mL). The mixture was extracted with ethyl acetate (2×500 mL). and washed with brine (2×500 mL). The mixture was dried over anhydrous Na₂SO₄. After filtration, the filtrate was concentrated under vacuum. The crude residue was purified onto a silica gel column and eluted with ethyl acetate/petroleum ether=1:5 to give 4-{5-bromo-4,4-dimethyl-2H,3H-pyrano[2,3-c]pyridin-8-yl}morpholine as a yellow oil (1.1 g, 64.2%).

¹H NMR (300 MHz, DMSO-d₆) δ 7.84 (s, 1H), 4.23-4.11 (m, 2H), 3.75-3.63 (m, 4H), 3.21 (t, J=4.5 Hz, 4H), 1.87 (t, J=5.1 Hz, 2H), 1.51 (s, 6H).

Synthesis of 4-{5-ethenyl-4,4-dimethyl-2H,3H-pyrano[2,3-c]pyridin-8-yl}morpholine: Into a 40 mL sealed tube, were added 4-{5-bromo-4,4-dimethyl-2H,3H-pyrano[2,3-c]pyridin-8-yl}morpholine (800 mg, 2.4 mmol, 1.0 eq), dioxane:H₂O=20:1 (8 mL), 2-ethenyl-4,4,5,5-tetramethyl-1,3,2-dioxaborolane (377 mg, 2.4 mmol, 1.0 eq), K₂CO₃ (1.1 g, 7.3 mmol, 3.0 eq), Pd-PEPPSI-IPent (80 mg, 10%). The resulting mixture was stirred for 3 hours at 80° C. under nitrogen atmosphere. The resulting mixture was filtered, the filter cake was washed with ethyl acetate (3×100 mL). The resulting mixture was extracted with ethyl acetate (3×100 mL). The combined organic layers were dried over anhydrous Na₂SO₄. After filtration, the filtrate was concentrated under vacuum. The crude residue was applied onto a silica gel column and eluted with petroleum ether/ethyl acetate=3:1 to give 4-{5-ethenyl-4,4-dimethyl-2H,3H-pyrano[2,3-c]pyridin-8-yl}morpholine as a light yellow oil (260 mg, 38.8%). ¹H NMR (400 MHz, DMSO-d₆) δ 7.70 (s, 1H), 7.12 (dd, J=17.2, 10.8 Hz, 1H), 5.43 (d, J=17.2 Hz, 1H), 5.27-5.17 (m, 1H), 4.15 (t, J=5.4 Hz, 2H), 3.69 (t, J=4.5 Hz, 4H), 3.21 (t, J=4.8 Hz, 4H), 1.80 (t, J=5.4 Hz, 2H), 1.37 (s, 6H).

Synthesis of 4,4-dimethyl-8-(morpholin-4-yl)-2H,3H-pyrano[2,3-c]pyridine-5-carbaldehyde: Into an 8 mL sealed tube, were added 4-{5-ethenyl-4,4-dimethyl-2H,3H-pyrano[2,3-c]pyridin-8-yl}morpholine (290 mg, 1.1 mmol, 1.0 eq), t-BuOH:THF:H₂O=6:6:1 (3 mL), NalO₄ (678 mg, 3.2 mmol, 3.0 eq), K₂OsO₄.2H₂O (273 mg, 0.7 mmol, 0.7 eq). The resulting mixture was stirred for 3 hours at 25° C. under nitrogen atmosphere. The resulting mixture was filtered, the filter cake was washed with ethyl acetate (3×100 mL). The resulting mixture was extracted with ethyl acetate (3×100 mL). The combined organic layers were dried over anhydrous Na₂SO₄. After filtration, the filtrate was concentrated under vacuum. The crude residue was applied onto a silica gel column and eluted with petroleum ether/ethyl acetate=3:1 to give 4,4-dimethyl-8-(morpholin-4-yl)-2H,3H-pyrano[2,3-c]pyridine-5-carbaldehyde as a light yellow oil (170 mg, 58.2%). ¹H NMR (400 MHz, DMSO-d₆) δ 10.10 (s, 1H), 8.21 (s, 1H), 4.22-4.17 (m, 2H), 3.72-3.66 (m, 4H), 3.55 (t, J=4.6 Hz, 4H), 1.87-1.79 (m, 2H), 1.45 (s, 6H).

Synthesis of 4-(4,4-dimethyl-5-{[(3R)-3-(2-methylphenyl)piperazin-1-yl]methyl}-2H,3H-pyrano[2,3-c]pyridin-8-yl)morpholine: Into an 8-mL sealed tube, were placed 4,4-dimethyl-8-(morpholin-4-yl)-2H,3H-pyrano[2,3-c]pyridine-5-carbaldehyde (50 mg, 0.2 mmol, 1.0 eq), (2R)-2-(2-methylphenyl)piperazine (32 mg, 0.2 mmol, 1.0 eq), NaBH₃CN (34 mg, 0.5 mmol, 3.0 eq), ZnCl₂ (74 mg, 0.5 mmol, 3.0 eq), MeOH (0.5 mL). The resulting solution was stirred for 1.5 hours at 25° C. The resulting mixture was then quenched by the addition of water (30 mL). The resulting solution was extracted with dichloromethane (2×30 mL) and washed with brine (2×30 mL) and the organic layers combined. The mixture was dried over anhydrous Na₂SO₄. After filtration, the filtrate was concentrated under vacuum. The crude residue was applied onto a silica gel column and eluted with dichloromethane/methanol=10:1 to give 4-(4,4-dimethyl-5-{[(3R)-3-(2-methylphenyl)piperazin-1-yl]methyl}-2H,3H-pyrano[2,3-c]pyridin-8-yl)morpholine as a light yellow solid (50 mg, 63.3%). LC-MS (ES, m/z) M+1: 437.

Synthesis of 4-{2-[(2R)-4-{[4,4-dimethyl-8-(morpholin-4-yl)-2H,3H-pyrano[2,3-c]pyridin-5-yl]methyl}-2-(2-methylphenyl)piperazin-1-yl]-7-azaspiro[3.5]nonan-7-yl}-2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-N-[(3R)-5-nitro-3-(oxan-4-yl)-3,4-dihydro-2H-1,4-benzoxazin-7-ylsulfonyl]benzamide: Into an 8-mL sealed-tube, were placed 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-N-[(3R)-5-nitro-3-(oxan-4-yl)-3,4-dihydro-2H-1,4-benzoxazin-7-ylsulfonyl]-4-{2-oxo-7-azaspiro[3.5]nonan-7-yl}benzamide (20 mg, 0.03 mmol, 1.0 eq), 4-(4,4-dimethyl-5-{[(3R)-3-(2-methylphenyl)piperazin-1-yl]methyl}-2H,3H-pyrano[2,3-c]pyridin-8-yl)morpholine (14 mg, 0.03 mmol, 1.3 eq), MeOH (1 mL), ZnCl₂ (10 mg, 0.08 mmol, 3.0 eq), NaBH₃CN (5 mg, 0.08 mmol, 3.0 eq). The resulting solution was stirred for 4 hours at 70° C. The resulting mixture was then quenched by the addition of water (20 mL). The resulting solution was extracted with dichloromethane/methanol=10:1 (2×30 mL) and washed with brine (2×20 mL) and the organic layers combined. The mixture was dried over anhydrous Na₂SO₄. After filtration, the filtrate was concentrated under vacuum. The residue was purified by Prep-TLC (dichloromethane/methanol=15:1) to give 4-{2-[(2R)-4-{[4,4-dimethyl-8-(morpholin-4-yl)-2H,3H-pyrano[2,3-c]pyridin-5-yl]methyl}-2-(2-methylphenyl)piperazin-1-yl]-7-azaspiro[3.5]nonan-7-yl}-2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-N-[(3R)-5-nitro-3-(oxan-4-yl)-3,4-dihydro-2H-1,4-benzoxazin-7-ylsulfonyl]benzamide as a yellow solid (8 mg, 26.4%). LC-MS (ES, m/z) M+1: 1235. ¹H NMR (400 MHz, Methanol-d₄) δ 8.02 (d, J=2.0 Hz, 1H), 7.87 (d, J=9.2 Hz, 1H), 7.79 (s, 1H), 7.49-7.45 (m, 1H), 7.22 (d, J=2.2 Hz, 1H), 7.16-7.13 (m, 3H), 7.03 (d, J=3.6 Hz, 1H), 6.99-6.89 (m, 1H), 6.70 (d, J=2.4 Hz, 1H), 6.49 (s, 1H), 5.99 (d, J=3.4 Hz, 1H), 4.33-4.29 (m, 2H), 4.26-4.22 (m, 3H), 4.15-4.10 (m, 1H), 4.03-3.98 (m, 2H), 3.96-3.90 (m, 1H), 3.80-3.78 (m, 4H), 3.74-3.61 (m, 4H), 3.53-3.38 (m, 5H), 3.25-3.21 (m, 4H), 3.18-3.10 (m, 6H), 3.02-2.96 (m, 1H), 2.69-2.63 (m, 1H), 2.45-2.29 (m, 7H), 1.91-1.84 (m, 3H), 1.78-1.67 (m, 4H), 1.55-1.48 (m, 6H), 1.46-1.34 (m, 5H), 1.33-1.28 (m, 4H).

### Example 193 Preparation of 4-(2-((R)-4-((S)-1-(5-methoxy-6-((S)-3-methylmorpholino)pyridin-3-yl)ethyl)-2-(o-tolyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide (assumed)

Synthesis of (S)-4-(3-methoxy-5-((S)-1-((R)-3-(o-tolyl)piperazin-1-yl)ethyl)pyridin-2-yl)-3-methylmorpholine (assumed): Into a 20 mL sealed tube were added (R)-1-((S)-1-(6-chloro-5-methoxypyridin-3-yl)ethyl)-3-(o-tolyl)piperazine (assumed) (230 mg, 0.7 mmol, 1.0 eq), t-BuOK (149 mg, 1.3 mmol, 2.0 eq), Pd-PEPPSI-IPentCl (28 mg, 0.03 mmol, 0.05 eq), dioxane (4 mL) and (3S)-3-methylmorpholine (404 mg, 4.0 mmol, 6.0 eq) at 25° C. The resulting mixture was stirred for 16 hours at 110° C. under nitrogen atmosphere. The reaction was quenched by the addition of water (2 mL), and then extracted with EtOAc (2×10 mL). The combined organic layers were washed with brine (20 mL), dried over anhydrous Na₂SO₄. After filtration, the filtrate was concentrated under reduced pressure. The residue was purified by Prep-TLC (dichloromethane/methanol=10:1) to afford (S)-4-(3-methoxy-5-((S)-1-((R)-3-(o-tolyl)piperazin-1-yl)ethyl)pyridin-2-yl)-3-methylmorpholine (assumed) as a colorless oil (200 mg, 73.3%). LC-MS (ESI, m/z) M+1: 411.

Synthesis of 4-(2-((R)-4-((S)-1-(5-methoxy-6-((S)-3-methylmorpholino)pyridin-3-yl)ethyl)-2-(o-tolyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide (assumed): Into a 2-mL sealed-tube, were placed (S)-4-(3-methoxy-5-((S)-1-((R)-3-(o-tolyl)piperazin-1-yl)ethyl)pyridin-2-yl)-3-methylmorpholine (assumed) (10 mg, 0.03 mmol, 1.0 eq), 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-N-[(3R)-5-nitro-3-(oxan-4-yl)-3,4-dihydro-2H-1,4-benzoxazin-7-ylsulfonyl]-4-{2-oxo-7-azaspiro[3.5]nonan-7-yl}benzamide (20 mg, 0.025 mmol, 1.0 eq), NaBH₃CN (8 mg, 0.1 mmol, 5.0 eq), ZnCl₂ (17 mg, 0.1 mmol, 5.0 eq) and MeOH (0.5 mL). The resulting solution was stirred for 5 hours at 70° C. The resulting mixture was then quenched by the addition of water (5 mL), and then extracted with dichloromethane/methanol=10:1 (2×10 mL). The combined organic layers was washed with brine (2×10 mL), dried over anhydrous Na₂SO₄. After filtration, the filtrate was concentrated under vacuum. The crude product was purified by Prep-HPLC using the following conditions (Column: XBridge Prep OBD C18 Column, 30×150 mm, 5 μm; Mobile Phase A: water (0.05% NH3·H2O), Mobile Phase B: MeCN; Flow rate: 60 mL/min; Gradient: 25% B to 55% B in 8 min, 55% B; Wave Length: 254/220 nm;) to afford 4-(2-((R)-4-((S)-1-(5-methoxy-6-((S)-3-methylmorpholino)pyridin-3-yl)ethyl)-2-(o-tolyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide (assumed) as a light yellow solid (3 mg, 10.1%). LC-MS (ESI, m/z) M+1:1209. ¹H NMR (400 MHz, DMSO-d₆) δ 12.75 (s, 1H), 11.14 (s, 1H), 8.85 (s, 1H), 7.97-7.95 (m, 1H), 7.76-7.74 (m, 1H), 7.68-7.66 (m, 1H), 7.35-7.30 (m, 2H), 7.14-7.12 (m, 1H), 7.09-7.03 (m, 4H), 6.98-6.95 (m, 1H), 6.79-6.75 (m, 1H), 6.43-6.40 (m, 1H), 5.99-5.95 (m, 1H), 4.29-4.25 (m, 1H), 4.21-4.18 (m, 1H), 4.05-4.02 (m, 3H), 3.87-3.48 (m, 11H), 3.16-2.64 (m, 11H), 2.45-2.22 (m, 9H), 2.09-2.05 (m, 3H), 1.76-1.73 (m, 1H), 1.63-1.60 (m, 4H), 1.26-1.23 (m, 10H), 1.11-1.08 (m, 2H), 1.00-0.95 (m, 3H), 0.86-0.83 (m, 1H).

### Example 194 Preparation of 4-(2-((R)-4-((R)-1-(5-methoxy-6-((S)-3-methylmorpholino)pyridin-3-yl)ethyl)-2-(o-tolyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide (assumed)

Synthesis of (S)-4-(3-methoxy-5-((R)-1-((R)-3-(o-tolyl)piperazin-1-yl)ethyl)pyridin-2-yl)-3-methylmorpholine (assumed): Into a 20 mL sealed tube were added (R)-1-((R)-1-(6-chloro-5-methoxypyridin-3-yl)ethyl)-3-(o-tolyl)piperazine (230 mg, 0.7 mmol, 1.0 eq), t-BuOK (149 mg, 1.3 mmol, 2.0 eq), Pd-PEPPSI-IPentCl (28 mg, 0.03 mmol, 0.05 eq), dioxane (4 mL) and (3S)-3-methylmorpholine (404 mg, 4.0 mmol, 6.0 eq) at 25° C. The resulting mixture was stirred for 16 hours at 110° C. under nitrogen atmosphere. The reaction was quenched by the addition of water (2 mL), and then extracted with EtOAc (2×10 mL). The combined organic layers were washed with brine (20 mL), dried over anhydrous Na₂SO₄. After filtration, the filtrate was concentrated under reduced pressure. The residue was purified by Prep-TLC (dichloromethane/methanol=10:1) to afford (S)-4-(3-methoxy-5-((R)-1-((R)-3-(o-tolyl)piperazin-1-yl)ethyl)pyridin-2-yl)-3-methylmorpholine (assumed) as a colorless oil (200 mg, 73.3%). LC-MS (ESI, m/z) M+1: 411.

Synthesis of 4-(2-((R)-4-((R)-1-(5-methoxy-6-((S)-3-methylmorpholino)pyridin-3-yl)ethyl)-2-(o-tolyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide (assumed): Into a 2-mL sealed-tube, were placed (S)-4-(3-methoxy-5-((R)-1-((R)-3-(o-tolyl)piperazin-1-yl)ethyl)pyridin-2-yl)-3-methylmorpholine (assumed) (10 mg, 0.03 mmol, 1.0 eq), 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-N-[(3R)-5-nitro-3-(oxan-4-yl)-3,4-dihydro-2H-1,4-benzoxazin-7-ylsulfonyl]-4-{2-oxo-7-azaspiro[3.5]nonan-7-yl}benzamide (20 mg, 0.025 mmol, 1.0 eq), NaBH₃CN (8 mg, 0.1 mmol, 5.0 eq), ZnCl₂ (17 mg, 0.1 mmol, 5.0 eq) and MeOH (0.5 mL). The resulting solution was stirred for 5 hours at 70° C. The resulting mixture was then quenched by the addition of water (5 mL), and then extracted with dichloromethane/methanol=10:1 (2×10 mL). The combined organic layers was washed with brine (2×10 mL), dried over anhydrous Na₂SO₄. After filtration, the filtrate was concentrated under vacuum. The crude product was purified by Prep-HPLC using the following conditions (Column: XBridge Prep OBD C18 Column, 30×150 mm, 5 μm; Mobile Phase A: water (0.05% NH3·H2O), Mobile Phase B: MeCN; Flow rate: 60 mL/min; Gradient: 25% B to 55% B in 8 min, 55% B; Wave Length: 254/220 nm;) to afford 4-(2-((R)-4-((R)-1-(5-methoxy-6-((S)-3-methylmorpholino)pyridin-3-yl)ethyl)-2-(o-tolyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide (assumed) as a light yellow solid (3 mg, 10.1%). LC-MS (ESI, m/z) M+1: 1209. ¹H NMR (400 MHz, DMSO-d₆) δ 12.76 (s, 1H), 11.06 (s, 1H), 8.75 (s, 1H), 8.01-7.95 (m, 1H), 7.76-7.74 (m, 1H), 7.68-7.64 (m, 1H), 7.33-7.28 (m, 2H), 7.12-7.10 (m, 1H), 7.09-7.03 (m, 4H), 6.98-6.95 (m, 1H), 6.79-6.75 (m, 1H), 6.43-6.40 (m, 1H), 5.99-5.95 (m, 1H), 4.29-4.25 (m, 1H), 4.21-4.18 (m, 1H), 4.05-4.02 (m, 3H), 3.87-3.48 (m, 11H), 3.16-2.64 (m, 11H), 2.45-2.22 (m, 9H), 2.09-2.05 (m, 3H), 1.76-1.73 (m, 1H), 1.63-1.60 (m, 4H), 1.26-1.23 (m, 10H), 1.11-1.08 (m, 2H), 1.09-1.00 (m, 3H), 0.86-0.83 (m, 1H).

### Example 195 Preparation of 4-{2-[(2R)-4-[(4R)-7,8-difluoro-3,4-dihydro-2H-1-benzopyran-4-yl]-2-(2-methylphenyl)piperazin-1-yl]-7-azaspiro[3.5]nonan-7-yl}-2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-N-[(3R)-5-nitro-3-(oxan-4-yl)-3,4-dihydro-2H-1,4-benzoxazin-7-ylsulfonyl]benzamide (assumed)

Synthesis of (3R)-1-[(4S)-7,8-difluoro-3,4-dihydro-2H-1-benzopyran-4-yl]-3-(2-methylphenyl)piperazine (assumed) and (3R)-1-[(4R)-7,8-difluoro-3,4-dihydro-2H-1-benzopyran-4-yl]-3-(2-methylphenyl)piperazine (assumed): Into a 40 mL vial were added 4-chloro-7,8-difluoro-3,4-dihydro-2H-1-benzopyran (600 mg, 2.9 mmol, 1.0 eq), (2R)-2-(2-methylphenyl)piperazine (517 mg, 2.9 mmol, 1.0 eq), K₂CO₃ (1216 mg, 8.8 mmol, 3.0 eq) and MeCN (8 mL). The resulting mixture was stirred for overnight at 80° C. The resulting mixture was diluted with water (10 mL) and extracted with EtOAc (3×10 mL). The combined organic layers were washed with brine (10 mL), dried over anhydrous Na₂SO₄. After filtration, the filtrate was concentrated under vacuum. The residue was purified by Prep-TLC (EA 100%) to give (3R)-1-[(4S)-7,8-difluoro-3,4-dihydro-2H-1-benzopyran-4-yl]-3-(2-methylphenyl)piperazine (assumed) (55 mg, 5.5%) and (3R)-1-[(4R)-7,8-difluoro-3,4-dihydro-2H-1-benzopyran-4-yl]-3-(2-methylphenyl)piperazine (assumed) as a colorless solid (210 mg, 20.8%). LC-MS (ES, m/z) M+1: 345.

Synthesis of 4-{2-[(2R)-4-[(4R)-7,8-difluoro-3,4-dihydro-2H-1-benzopyran-4-yl]-2-(2-methylphenyl)piperazin-1-yl]-7-azaspiro[3.5]nonan-7-yl}-2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-N-[(3R)-5-nitro-3-(oxan-4-yl)-3,4-dihydro-2H-1,4-benzoxazin-7-ylsulfonyl]benzamide (assumed): Into an 8 mL vial were added (3R)-1-[(4R)-7,8-difluoro-3,4-dihydro-2H-1-benzopyran-4-yl]-3-(2-methylphenyl)piperazine (assumed) (20 mg, 0.06 mmol, 1.0 eq), 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-N-[(3R)-5-nitro-3-(oxan-4-yl)-3,4-dihydro-2H-1,4-benzoxazin-7-ylsulfonyl]-4-{2-oxo-7-azaspiro[3.5]nonan-7-yl}benzamide (47 mg, 0.06 mmol, 1.0 eq), NaBH₃CN (18 mg, 0.3 mmol, 5.0 eq), ZnCl₂ (40 mg, 0.3 mmol, 5.0 eq) and MeOH (3 mL). The resulting mixture was stirred for overnight at 70° C. The reaction was quenched by the addition of water (3 mL) and extracted with CH₂Cl₂ (3×10 mL). The combined organic layers were washed with brine (10 mL), dried over anhydrous Na₂SO₄. After filtration, the filtrate was concentrated under vacuum. The residue was purified by Prep-TLC (CH₂Cl₂/MeOH=15:1) to give 4-{2-[(2R)-4-[(4R)-7,8-difluoro-3,4-dihydro-2H-1-benzopyran-4-yl]-2-(2-methylphenyl)piperazin-1-yl]-7-azaspiro[3.5]nonan-7-yl}-2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-N-[(3R)-5-nitro-3-(oxan-4-yl)-3,4-dihydro-2H-1,4-benzoxazin-7-ylsulfonyl]benzamide (assumed) as a yellow solid (8 mg, 12.1%). LC-MS (ES, m/z) M+1: 1144. ¹H NMR (300 MHz, Chloroform-d) δ 12.47 (s, 1H), 9.31 (s, 1H), 8.46 (s, 1H), 8.31 (d, J=2.1 Hz, 1H), 8.13 (d, J=9.1 Hz, 1H), 7.93 (s, 1H), 7.45 (s, 1H), 7.19 (d, J=33.9 Hz, 3H), 6.86 (d, J=9.3 Hz, 1H), 6.72 (d, J=12.9 Hz, 2H), 6.43 (s, 1H), 6.15 (s, 1H), 4.47-4.41 (m, 4H), 4.34-3.94 (m, 7H), 3.81-3.57 (m, 3H), 3.57-3.18 (m, 8H), 3.01-2.86 (m, 7H), 2.44 (s, 3H), 2.28-2.16 (m, 3H), 2.05-2.02 (m, 8H), 1.89-1.39 (m, 4H).

### Example 196 Preparation of 4-{2-[(2R)-4-[(4S)-7,8-difluoro-3,4-dihydro-2H-1-benzopyran-4-yl]-2-(2-methylphenyl)piperazin-1-yl]-7-azaspiro[3.5]nonan-7-yl}-2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-N-[(3R)-5-nitro-3-(oxan-4-yl)-3,4-dihydro-2H-1,4-benzoxazin-7-ylsulfonyl]benzamide (assumed)

Synthesis of 4-{2-[(2R)-4-[(4S)-7,8-difluoro-3,4-dihydro-2H-1-benzopyran-4-yl]-2-(2-methylphenyl)piperazin-1-yl]-7-azaspiro[3.5]nonan-7-yl}-2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-N-[(3R)-5-nitro-3-(oxan-4-yl)-3,4-dihydro-2H-1,4-benzoxazin-7-ylsulfonyl]benzamide (assumed): Into an 8 mL vial were added (3R)-1-[(4S)-7,8-difluoro-3,4-dihydro-2H-1-benzopyran-4-yl]-3-(2-methylphenyl)piperazine (assumed) (20 mg, 0.06 mmol, 1.0 eq), 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-N-[(3R)-5-nitro-3-(oxan-4-yl)-3,4-dihydro-2H-1,4-benzoxazin-7-ylsulfonyl]-4-{2-oxo-7-azaspiro[3.5]nonan-7-yl}benzamide (47 mg, 0.06 mmol, 1.0 eq), NaBH₃CN (18 mg, 0.3 mmol, 5.0 eq), ZnCl₂ (40 mg, 0.3 mmol, 5.0 eq) and MeOH (3 mL). The resulting mixture was stirred for overnight at 70° C. The reaction was quenched by the addition of water (5 mL) and extracted with CH₂Cl₂ (3×10 mL). The combined organic layers were washed with brine (10 mL), dried over anhydrous Na₂SO₄. After filtration, the filtrate was concentrated under vacuum. The residue was purified by Prep-TLC (CH₂Cl₂/MeOH=10:1) to give 4-{2-[(2R)-4-[(4S)-7,8-difluoro-3,4-dihydro-2H-1-benzopyran-4-yl]-2-(2-methylphenyl)piperazin-1-yl]-7-azaspiro[3.5]nonan-7-yl}-2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-N-[(3R)-5-nitro-3-(oxan-4-yl)-3,4-dihydro-2H-1,4-benzoxazin-7-ylsulfonyl]benzamide (assumed) as a yellow solid (7 mg, 10.6%). LC-MS (ES, m/z) M+1: 1143. ¹H NMR (300 MHz, Chloroform-d) δ 12.63 (s, 1H), 8.45 (s, 1H), 8.37-8.26 (m, 2H), 8.13 (d, J=9.2 Hz, 1H), 7.52-7.39 (m, 2H), 7.09 (dd, J=7.0, 4.8 Hz, 3H), 6.86 (d, J=9.1 Hz, 1H), 6.68 (d, J=11.2 Hz, 2H), 6.45 (s, 1H), 6.14 (s, 1H), 4.60-4.38 (m, 3H), 4.26-3.91 (m, 6H), 3.84-3.72 (m, 2H), 3.45-3.35 (m, 6H), 3.09 (s, 5H), 2.98-2.79 (m, 3H), 2.42-2.38 (m, 1H), 2.31-2.16 (m, 4H), 2.02-1.91 (m, 3H), 1.79-1.75 (m, 4H), 1.70-1.66 (m, 4H), 1.44-1.29 (m, 6H).

### Example 197 Preparation of 4-(2-((R)-4-((S)-6,8-difluorochroman-4-yl)-2-(o-tolyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide (assumed)

Synthesis of (R)-1-((S)-6,8-difluorochroman-4-yl)-3-(o-tolyl)piperazine (assumed) and (R)-1-((R)-6,8-difluorochroman-4-yl)-3-(o-tolyl)piperazine (assumed): Into a 40 mL vial were added 4-chloro-6,8-difluorochromane (500 mg, 2.4 mmol, 1.0 eq), (2R)-2-(2-methylphenyl)piperazine (assumed) (430 mg, 2.4 mmol, 1.0 eq), K₂CO₃ (1013 mg, 7.3 mmol, 3.0 eq) and MeCN (6 mL). The resulting mixture was stirred for additional 4 hours at 80° C. The reaction was quenched with water, and then extracted with CH₂Cl₂ (5 mL). The combined organic layers were washed with brine (3×5 mL), dried over anhydrous Na₂SO₄. After filtration, the filtrate was concentrated under vacuum. The crude residue was purified by Prep-TLC (EA) to give (R)-1-((S)-6,8-difluorochroman-4-yl)-3-(o-tolyl)piperazine (assumed) as a light yellow solid (45 mg, 5.3%) and (R)-1-((R)-6,8-difluorochroman-4-yl)-3-(o-tolyl)piperazine (assumed) as a light yellow solid (150 mg, 17.8%). LC-MS (ES, m/z) M+1: 345.

Synthesis of 4-(2-((R)-4-((S)-6,8-difluorochroman-4-yl)-2-(o-tolyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide (assumed): Into an 8 mL vial were added (3R)-1-[(4S)-6,8-difluoro-3,4-dihydro-2H-1-benzopyran-4-yl]-3-(2-methylphenyl)piperazine (assumed) (7 mg, 0.1 mmol, 1.0 eq), 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-N-[(3R)-5-nitro-3-(oxan-4-yl)-3,4-dihydro-2H-1,4-benzoxazin-7-ylsulfonyl]-4-{2-oxo-7-azaspiro[3.5]nonan-7-yl}benzamide (16 mg, 0.1 mmol, 1.0 eq), NaBH₃CN (6 mg, 0.1 mmol, 5.0 eq), ZnCl₂ (13 mg, 0.1 mmol, 5.0 eq) and MeOH (1 mL). The resulting mixture was stirred for additional 4 hours at 70° C. The reaction was quenched with water, and then extracted with CH₂Cl₂ (5 mL). The combined organic layers were washed with brine (3×5 mL), dried over anhydrous Na₂SO₄. After filtration, the filtrate was concentrated under vacuum. The crude residue was purified by Prep-TLC (CH₂Cl₂/MeOH=15:1) to give 4-(2-((R)-4-((S)-6,8-difluorochroman-4-yl)-2-(o-tolyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide (assumed) as a yellow solid (10 mg, 43.7%). LC-MS (ESI, m/z) M+1: 1144. ¹H NMR (300 MHz, Chloroform-d) δ 12.64 (s, 1H), 8.46 (s, 2H), 8.31 (d, J=2.1 Hz, 1H), 8.17-8.08 (m, 1H), 7.46 (d, J=2.2 Hz, 1H), 7.19-7.06 (m, 5H), 6.86-6.83 (m, 1H), 6.80-6.68 (m, 1H), 6.69 (s, 1H), 6.45 (d, J=2.4 Hz, 1H), 6.14-6.12 (m, 1H), 5.37 (s, 1H), 4.60-4.39 (m, 3H), 4.18-4.02 (m, 5H), 4.02-3.85 (m, 2H), 3.59-3.57 (m, 1H), 3.50-3.48 (m, 1H), 3.45-3.42 (m, 4H), 3.39-3.36 (m, 2H), 3.09-3.05 (m, 4H), 2.97-2.96 (m, 2H), 2.78-2.75 (m, 1H), 2.67-2.54 (m, 2H), 2.49 (s, 1H), 2.42-2.40 (m 3H), 2.30-2.09 (m, 2H), 2.05 (s, 1H), 2.00 (s, 1H), 1.80 (s, 3H), 1.78-1.67 (m, 2H), 1.63 (s, 2H), 1.35-1.33 (m, 1H), 1.28 (s, 3H), 0.89-0.86 (m, 2H).

### Example 198 Preparation of 4-(2-((R)-4-((R)-6,8-difluorochroman-4-yl)-2-(o-tolyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide (assumed)

Synthesis of 4-(2-((R)-4-((R)-6,8-difluorochroman-4-yl)-2-(o-tolyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide (assumed): Into an 8 mL vial were added (3R)-1-[(4R)-6,8-difluoro-3,4-dihydro-2H-1-benzopyran-4-yl]-3-(2-methylphenyl)piperazine (assumed) (7 mg, 0.1 mmol, 1.0 eq), 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-N-[(3R)-5-nitro-3-(oxan-4-yl)-3,4-dihydro-2H-1,4-benzoxazin-7-ylsulfonyl]-4-{2-oxo-7-azaspiro[3.5]nonan-7-yl}benzamide (16 mg, 0.1 mmol, 1.0 eq), NaBH₃CN (6 mg, 0.1 mmol, 5.0 eq), ZnCl₂ (13 mg, 0.1 mmol, 5.0 eq) and MeOH (1 mL). The resulting mixture was stirred for additional 4 hours at 70° C. The reaction was quenched with water, and then extracted with CH₂Cl₂ (5 mL). The organic layer was washed with brine (3×5 mL), dried over anhydrous Na₂SO₄. After filtration, the filtrate was concentrated under vacuum. The crude residue was purified by Prep-TLC (CH₂Cl₂/MeOH=15:1) to give 4-(2-((R)-4-((R)-6,8-difluorochroman-4-yl)-2-(o-tolyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide (assumed) as a yellow solid (10 mg, 43.7%). LC-MS (ESI, m/z) M+1: 1143. ¹H NMR (400 MHz, Chloroform-d) δ 12.63 (s, 1H), 8.45 (s, 2H), 8.31 (d, J=2.1 Hz, 1H), 8.17-8.10 (m, 1H), 7.46 (d, J=2.2 Hz, 1H), 7.41 (s, 1H), 7.16-7.15 (m, 1H), 7.11-7.10 (m, 4H), 7.06 (s, 1H), 6.86-6.84 (m, 1H), 6.71-6.70 (m 2H), 6.45-6.43 (m 1H), 6.17-6.11 (m, 1H), 5.37 (s, 1H), 4.59-4.45 (m, 1H), 4.48-4.38 (m, 2H), 4.21-3.90 (m, 4H), 3.45-3.41 (m, 7H), 3.30-3.18 (m, 1H), 3.14-3.05 (m, 1H), 2.98-2.83 (m, 2H), 2.36-2.33 (m, 2H), 2.28-2.26 (m, 5H), 2.14-2.01 (m, 1H), 2.05 (s, 2H), 1.81-1.74 (m, 6H), 1.66-1.63 (m, 5H), 1.43-1.40 (m, 1H), 1.28 (s, 3H), 1.24 (s, 1H), 0.94-0.87 (m, 1H), 0.86 (s, 1H).

### Example 199 Preparation of 4-(2-((R)-4-((1r,4R)-4-hydroxy-4-methylcyclohexyl)-2-(o-tolyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide (assumed)

Synthesis of (1R,4r)-1-methyl-4-((R)-3-(o-tolyl)piperazin-1-yl)cyclohexan-1-ol (assumed) and (1S,4s)-1-methyl-4-((R)-3-(o-tolyl)piperazin-1-yl)cyclohexan-1-ol (assumed): Into a 40 mL vial were added (2R)-2-(2-methylphenyl)piperazine (300 mg, 1.7 mmol, 1.0 eq), 4-hydroxy-4-methylcyclohexan-1-one (218 mg, 1.7 mmol, 1.0 eq), ZnCl₂ (1159 mg, 8.5 mmol, 5.0 eq), NaBH₃CN (534 mg, 8.5 mmol, 5.0 eq) and MeOH (3 mL). The resulting mixture was stirred for additional 16 hours at 70° C. The reaction was quenched with water, and then extracted with CH₂Cl₂ (5 mL). The combined organic layers were washed with brine (3×5 mL), dried over anhydrous Na₂SO₄. After filtration, the filtrate was concentrated under vacuum. The crude product (300 mg) was purified by Prep-HPLC using the following conditions (CH₂Cl₂/MeOH=13:1) to give (1R,4r)-1-methyl-4-((R)-3-(o-tolyl)piperazin-1-yl)cyclohexan-1-ol (assumed) (35 mg, 7.1%) and (1S,4s)-1-methyl-4-((R)-3-(o-tolyl)piperazin-1-yl)cyclohexan-1-ol as a light yellow solid (assumed) (90 mg, 18.3%). LC-MS (ES, m/z) M+1: 289.

Synthesis of 4-(2-((R)-4-((1r,4R)-4-hydroxy-4-methylcyclohexyl)-2-(o-tolyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide (assumed): Into an 8 mL vial were added (1R,4r)-1-methyl-4-((R)-3-(o-tolyl)piperazin-1-yl)cyclohexan-1-ol (assumed) (7 mg, 0.1 mmol, 1.0 eq), 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-N-[(3R)-5-nitro-3-(oxan-4-yl)-3,4-dihydro-2H-1,4-benzoxazin-7-ylsulfonyl]-4-{2-oxo-7-azaspiro[3.5]nonan-7-yl}benzamide (19 mg, 0.1 mmol, 1.0 eq), NaBH₃CN (7 mg, 0.1 mmol, 5.0 eq), ZnCl₂ (16 mg, 0.1 mmol, 5.0. eq) and MeOH (2 mL). The resulting mixture was stirred for additional 4 hours at 25° C. The reaction was quenched with water at 25° C. The resulting mixture was extracted with CH₂Cl₂ (5 mL). The combined organic layers were washed with brine (3×5 mL), dried over anhydrous Na₂SO₄. After filtration, the filtrate was concentrated under vacuum. The crude residue was purified by Prep-TLC (CH₂Cl₂/MeOH=15:1) to give 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-4-{2-[(2R)-2-(2-methylphenyl)-4-[(1r,4r)-4-hydroxy-4-methylcyclohexyl]piperazin-1-yl]-7-azaspiro[3.5]nonan-7-yl}-N-[(3R)-5-nitro-3-(oxan-4-yl)-3,4-dihydro-2H-1,4-benzoxazin-7-ylsulfonyl]benzamide (assumed) as a yellow solid (10 mg, 37.9%). LC-MS (ESI, m/z) M+1: 1087. ¹H NMR (400 MHz, Chloroform-d) δ 12.57 (s, 1H), 10.06 (s, 1H), 8.34 (s, 1H), 8.13-8.07 (m, 1H), 7.90-7.85 (m, 1H), 7.46 (s, 1H), 7.33-7.24 (m, 3H), 7.12 (s, 1H), 7.03-7.00 (m, 3H), 6.93-6.88 (m, 1H), 6.68-6.63 (m, 1H), 6.44 (d, J=3.8 Hz, 1H), 6.31 (s, 1H), 5.89 (s, 1H), 4.38 (s, 1H), 4.30 (s, 1H), 4.23 (d, J=10.4 Hz, 1H), 3.94 (s, 1H), 3.86-3.84 (m, 3H), 3.77 (s, 1H), 3.44-3.41 (m, 8H), 3.24-3.20 (m, 5H), 3.03 (s, 1H), 2.94-2.91 (m, 3H), 2.88-2.86 (m, 3H), 2.46-2.39 (m, 6H), 2.25-2.23 (m, 3H), 1.92-1.90 (m, 3H), 1.78 (s, 1H), 1.32-1.30 (m, 7H), 1.11-1.04 (m, 4H), 0.93 (s, 1H).

### Example 200 Preparation of 4-(2-((R)-4-((1s,4S)-4-hydroxy-4-methylcyclohexyl)-2-(o-tolyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide (assumed)

Synthesis of 4-(2-((R)-4-((1s,4S)-4-hydroxy-4-methylcyclohexyl)-2-(o-tolyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide (assumed): Into an 8 mL vial were added (1S,4s)-1-methyl-4-((R)-3-(o-tolyl)piperazin-1-yl)cyclohexan-1-ol (assumed) (7 mg, 0.1 mmol, 1.0 eq), 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-N-[(3R)-5-nitro-3-(oxan-4-yl)-3,4-dihydro-2H-1,4-benzoxazin-7-ylsulfonyl]-4-{2-oxo-7-azaspiro[3.5]nonan-7-yl}benzamide (19 mg, 0.1 mmol, 1.0 eq), NaBH₃CN (7 mg, 0.1 mmol, 5.0 eq), ZnCl₂ (16 mg, 0.1 mmol, 5.0 eq) and MeOH (1 mL). The resulting mixture was stirred for additional 4 hours at 70° C. The reaction was quenched with water, and then extracted with CH₂Cl₂ (5 mL). The combined organic layers were washed with brine (3×5 mL), dried over anhydrous Na₂SO₄. After filtration, the filtrate was concentrated under vacuum. The crude residue was purified by Prep-TLC (CH₂Cl₂/MeOH=15:1) to give 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-4-{2-[(2R)-2-(2-methylphenyl)-4-[(1s,4s)-4-hydroxy-4-methylcyclohexyl]piperazin-1-yl]-7-azaspiro[3.5]nonan-7-yl}-N-[(3R)-5-nitro-3-(oxan-4-yl)-3,4-dihydro-2H-1,4-benzoxazin-7-ylsulfonyl]benzamide (assumed) as a yellow solid (10 mg, 37.9%). LC-MS (ESI, m/z) M+1: 1088. ¹H NMR (300 MHz, Chloroform-d) δ 12.56 (s, 1H), 10.07 (s, 1H), 8.32 (s, 1H), 8.09 (d, J=2.8 Hz, 1H), 7.89 (d, J=8.9 Hz, 1H), 7.54 (s, 3H), 7.32-7.25 (m, 4H), 7.05 (s, 4H), 6.89 (s, 1H), 6.67 (d, J=9.1 Hz, 1H), 6.42-6.40 (m, 1H), 6.31 (s, 1H), 5.87 (s, 1H), 4.26-4.22 (m, 1H), 4.20 (s, 3H), 3.88-3.86 (m, 9H), 3.74 (s, 1H), 3.35 (s, 1H), 3.27-3.17 (m, 13H), 3.08 (s, 1H), 2.90-2.85 (m, 4H), 2.41-2.38 (m, 4H), 2.26-2.24 (m, 3H), 1.78-1.75 (m, 6H), 1.46-1.44 (m, 2H), 1.02-1.00 (m, 1H), 0.91 (s, 1H).

### Example 201 Preparation of 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-4-{2-[(2R)-2-(2-methylphenyl)-4-[1-(oxan-4-yl)piperidin-4-yl]piperazin-1-yl]-7-azaspiro[3.5]nonan-7-yl}-N-[(3R)-5-nitro-3-(oxan-4-yl)-3,4-dihydro-2H-1,4-benzoxazin-7-ylsulfonyl]benzamide

Synthesis of 8-(oxan-4-yl)-1,4-dioxa-8-azaspiro[4.5]decane: Into a 100 mL round-bottom flask were added 1,4-dioxa-8-azaspiro[4.5]decane (10.0 g, 19.9 mmol, 1.0 eq), tetrahydro-4H-pyran-4-one (6.9 g, 19.9 mmol, 1.0 eq), NaBH₃CN (20.0 g, 99.8 mmol, 5.0 eq), ZnCl₂ (35.0 g, 99.8 mmol, 5.0 eq) and MeOH (20 mL). The resulting mixture was stirred for overnight at 50° C. The reaction was quenched by the addition of water (2 mL) and extracted with CH₂Cl₂ (3×5 mL). The combined organic layers were washed with brine (5 mL), dried over anhydrous Na₂SO₄. After filtration, the filtrate was concentrated under vacuum. The residue was purified by Prep-TLC (CH₂Cl₂/MeOH 10:1) to give 8-(oxan-4-yl)-1,4-dioxa-8-azaspiro[4.5]decane as a white oil (6.6 g, 57.2%). LC-MS (ES, m/z) M+1: 228.

Synthesis of 1-(oxan-4-yl)piperidin-4-one: Into a 100 mL round-bottom flask were added 8-(oxan-4-yl)-1,4-dioxa-8-azaspiro[4.5]decane (5.0 g, 4.4 mmol, 1.0 eq) and HCl (6 M) (50.0 mL, 43.9 mmol, 10.0 eq). The resulting mixture was stirred for additional overnight at 70° C. The residue was basified to pH=6 with saturated NaHCO₃ (aq.). The resulting mixture was extracted with CH₂Cl₂ (3×10 mL). The combined organic layers were washed with brine (3×10 mL), dried over anhydrous Na₂SO₄. After filtration, the filtrate was concentrated under vacuum. Finally, 1-(oxan-4-yl)piperidin-4-one was obtained as a white solid (1.3 g, 33.4%). LC-MS (ES, m/z) M+1: 184.

Synthesis of (3R)-3-(2-methylphenyl)-1-[1-(oxan-4-yl)piperidin-4-yl]piperazine: Into an 8 mL vial were added 1-(oxan-4-yl)piperidin-4-one (200 mg, 1.1 mmol, 1.0 eq), (2R)-2-(2-methylphenyl)piperazine (192 mg, 1.1 mmol, 1.0 equiv), NaBH₃CN (343 mg, 5.4 mmol, 5.0 eq), ZnCl₂ (743 mg, 5.4 mmol, 5.0 eq) and MeOH (3 mL). The resulting mixture was stirred for 3 hours at 25° C. The reaction was quenched by the addition of water (2 mL) and extracted with CH₂Cl₂ (3×5 mL). The combined organic layers were washed with brine (5 mL), dried over anhydrous Na₂SO₄. After filtration, the filtrate was concentrated under vacuum. The residue was purified by Prep-TLC (CH₂Cl₂/MeOH=8:1) to give (3R)-3-(2-methylphenyl)-1-[1-(oxan-4-yl)piperidin-4-yl]piperazine as a colorless oil (50 mg, 13.3%). LC-MS (ES, m/z) M+1: 344.

Synthesis of 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-4-{2-[(2R)-2-(2-methylphenyl)-4-[1-(oxan-4-yl)piperidin-4-yl]piperazin-1-yl]-7-azaspiro[3.5]nonan-7-yl}-N-[(3R)-5-nitro-3-(oxan-4-yl)-3,4-dihydro-2H-1,4-benzoxazin-7-ylsulfonyl]benzamide: Into an 8 mL vial were added (3R)-3-(2-methylphenyl)-1-[1-(oxan-4-yl)piperidin-4-yl]piperazine (50 mg, 0.15 mmol, 1.0 eq), 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-N-[(3R)-5-nitro-3-(oxan-4-yl)-3,4-dihydro-2H-1,4-benzoxazin-7-ylsulfonyl]-4-{2-oxo-7-azaspiro[3.5]nonan-7-yl}benzamide (118 mg, 0.15 mmol, 1.0 eq), NaBH₃CN (46 mg, 0.7 mmol, 5.0 eq), ZnCl₂ (99 mg, 0.7 mmol, 5.0 eq) and MeOH (2 mL). The resulting mixture was stirred for 8 hours at 70° C. The reaction was quenched by the addition of water (2 mL) and extracted with CH₂Cl₂ (3×5 mL). The combined organic layers were washed with brine (5 mL), dried over anhydrous Na₂SO₄. After filtration, the filtrate was concentrated under vacuum. The residue was purified by reversed-phase flash chromatography using the following conditions: column, C18 silica gel; mobile phase, MeCN in water (0.1% NH₃·H₂O), 10% to 50% gradient in 10 min; detector, UV 254 nm. Finally, 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-4-{2-[(2R)-2-(2-methylphenyl)-4-[1-(oxan-4-yl)piperidin-4-yl]piperazin-1-yl]-7-azaspiro[3.5]nonan-7-yl}-N-[(3R)-5-nitro-3-(oxan-4-yl)-3,4-dihydro-2H-1,4-benzoxazin-7-ylsulfonyl]benzamide was obtained as a yellow solid (5 mg, 3.0%). LC-MS (ES, m/z) M+1: 1141. ¹H NMR (300 MHz, Chloroform-d) δ 12.63 (s, 1H), 8.43 (d, J=10.0 Hz, 2H), 8.31 (s, 1H), 8.13 (d, J=9.1 Hz, 1H), 7.46 (s, 2H), 7.12 (d, J=12.1 Hz, 3H), 6.86 (d, J=9.5 Hz, 1H), 6.69 (s, 1H), 6.44 (s, 1H), 6.14 (s, 1H), 4.50-4.43 (m, 2H), 4.12-4.02 (m, 9H), 3.56-3.40 (m, 9H), 3.09-2.88 (m, 10H), 2.39-2.17 (m, 10H), 1.38-1.26 (m, 16H), 0.89 (m, 4H).

### Example 202 Preparation of 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-4-{2-[(2R)-2-(2-methylphenyl)-4-{3-oxaspiro[5.5]undecan-9-yl}piperazin-1-yl]-7-azaspiro[3.5]nonan-7-yl}-N-[(3R)-5-nitro-3-(oxan-4-yl)-3,4-dihydro-2H-1,4-benzoxazin-7-ylsulfonyl]benzamide

Synthesis of (3R)-3-(2-methylphenyl)-1-{3-oxaspiro[5.5]undecan-9-yl}piperazine: Into an 8 mL vial were added 3-oxaspiro[5.5]undecan-9-one (100 mg, 0.6 mmol, 1.0 eq), (2R)-2-(2-methylphenyl)piperazine (105 mg, 0.6 mmol, 1.0 eq), NaBH(OAc)₃ (630 mg, 3.0 mmol, 5.0 eq) and DCE (3 mL). The resulting mixture was stirred for 4 hours at 25° C. The residue was purified by Prep-TLC (CH₂Cl₂/MeOH=10:1) to give (3R)-3-(2-methylphenyl)-1-{3-oxaspiro[5.5]undecan-9-yl}piperazine (50 mg, 25.6%) as a colorless oil. LC-MS (ES, m/z) M+1: 329.

Synthesis of 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-4-{2-[(2R)-2-(2-methylphenyl)-4-{3-oxaspiro[5.5]undecan-9-yl}piperazin-1-yl]-7-azaspiro[3.5]nonan-7-yl}-N-[(3R)-5-nitro-3-(oxan-4-yl)-3,4-dihydro-2H-1,4-benzoxazin-7-ylsulfonyl]benzamide: Into an 8 mL vial were added (3R)-3-(2-methylphenyl)-1-{3-oxaspiro[5.5]undecan-9-yl}piperazine (20 mg, 0.06 mmol, 1.0 eq), 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-N-[(3R)-5-nitro-3-(oxan-4-yl)-3,4-dihydro-2H-1,4-benzoxazin-7-ylsulfonyl]-4-{2-oxo-7-azaspiro[3.5]nonan-7-yl}benzamide (50 mg, 0.06 mmol, 1.0 eq), NaBH₃CN (19 mg, 0.3 mmol, 5.0 eq), ZnCl₂ (41 mg, 0.3 mmol, 5.0 eq) and MeOH (3 mL). The resulting mixture was stirred for overnight at 70° C. The reaction was quenched by the addition of water (3 mL) and extracted with CH₂Cl₂ (3×10 mL). The combined organic layers were washed with brine (10 mL), dried over anhydrous Na₂SO₄. After filtration, the filtrate was concentrated under reduced pressure. The residue was purified by Prep-TLC (CH₂Cl₂/MeOH=15:1) to give 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-4-{2-[(2R)-2-(2-methylphenyl)-4-{3-oxaspiro[5.5]undecan-9-yl}piperazin-1-yl]-7-azaspiro[3.5]nonan-7-yl}-N-[(3R)-5-nitro-3-(oxan-4-yl)-3,4-dihydro-2H-1,4-benzoxazin-7-ylsulfonyl]benzamide as a yellow solid (6 mg, 8.8%). LC-MS (ES, m/z) M+1: 1127. ¹H NMR (400 MHz, Chloroform-d) δ 12.61 (s, 1H), 8.44 (s, 1H), 8.33 (s, 1H), 8.13 (d, J=9.1 Hz, 1H), 7.47 (d, J=2.1 Hz, 2H), 7.23-7.05 (m, 4H), 6.93-6.80 (m, 1H), 6.69 (s, 1H), 6.44 (d, J=2.4 Hz, 1H), 6.14 (d, J=3.0 Hz, 1H), 4.67-4.40 (m, 3H), 4.24-3.97 (m, 5H), 3.62-3.60 (m, 4H), 3.58-3.38 (m, 6H), 3.03-2.89 (m, 7H), 2.75 (s, 3H), 2.42-2.36 (m, 4H), 2.27-2.02 (m, 5H), 1.76-1.61 (m, 8H), 1.51-1.40 (m, 6H), 1.36-1.26 (m, 4H), 1.16-1.12 (m, 4H).

### Example 203 Preparation of 4-(2-((R)-4-((1S,4R)-4-((S)-3-methylmorpholino)cyclohexyl)-2-(o-tolyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide (assumed)

Synthesis of (S)-3-methyl-4-(1,4-dioxaspiro[4.5]decan-8-yl)morpholine: Into a 250 mL round-bottom flask were added (3S)-3-methylmorpholine (1.3 g, 12.7 mmol, 1.0 eq), 1,4-dioxaspiro[4.5]decan-8-one (1.9 g, 12.7 mmol, 1.0 eq), NaBH₃CN (4.1 g, 63.7 mmol, 5.0 eq), ZnCl₂ (8.6 g, 63.7 mmol, 5.0 eq) and MeOH (20 mL). The resulting mixture was stirred for additional 3 hours at 50° C. The reaction was quenched with water, and then extracted with CH₂Cl₂ (10 mL). The combined organic layers were washed with brine (3×5 mL), dried over anhydrous Na₂SO₄. After filtration, the filtrate was concentrated under vacuum. The crude residue was purified by silica gel column chromatography, eluted with ethyl acetate/petroleum ether=1:1 to give (S)-3-methyl-4-(1,4-dioxaspiro[4.5]decan-8-yl)morpholine as a light yellow oil (1.9 g, 61.3%). LC-MS (ES, m/z) M+1: 242.

Synthesis of (S)-4-(3-methylmorpholino)cyclohexan-1-one: Into a 100 mL round-bottom flask were added (S)-3-methyl-4-(1,4-dioxaspiro[4.5]decan-8-yl)morpholine (1.0 g, 4.1 mmol, 1 eq), HCl (5 mL) and MeCN (5 mL). The resulting mixture was stirred for additional 12 hours at 25° C. The reaction was quenched with water, and then neutralized to pH=7 with saturated NaHCO₃ (aq.). The resulting mixture was filtered, the filter cake was washed with CH₂Cl₂ (3×5 mL). The filtrate was concentrated under vacuum. The crude product mixture was used in the next step directly without further purification. LC-MS (ES, m/z) M+1: 198.

Synthesis of (S)-3-methyl-4-((1R,4S)-4-((R)-3-(o-tolyl)piperazin-1-yl)cyclohexyl)morpholine (assumed) and (S)-3-methyl-4-((1S,4R)-4-((R)-3-(o-tolyl)piperazin-1-yl)cyclohexyl)morpholine (assumed): Into a 40 mL vial were added 4-[(3S)-3-methylmorpholin-4-yl]cyclohexan-1-one (300 mg, 1.5 mmol, 1.0 eq), (2R)-2-(2-methylphenyl)piperazine (268 mg, 1.5 mmol, 1.0 eq), NaBH₃CN (477 mg, 7.6 mmol, 5.0 eq), ZnCl₂ (1036 mg, 7.6 mmol, 5.0 eq) and MeOH (3 mL). The resulting mixture was stirred for additional 4 hours at 50° C. The reaction was quenched with water at 25° C. The resulting mixture was extracted with CH₂Cl₂ (5 mL). The combined organic layers were washed with brine (3×5 mL), dried over anhydrous Na₂SO₄. After filtration, the filtrate was concentrated under vacuum. The crude product (260 mg) was purified by Prep-HPLC using the following conditions (CH₂Cl₂/MeOH=10:1) to afford (S)-3-methyl-4-((1R,4S)-4-((R)-3-(o-tolyl)piperazin-1-yl)cyclohexyl)morpholine (assumed) as a light yellow oil (30 mg, 5.5%) and (S)-3-methyl-4-((1S,4R)-4-((R)-3-(o-tolyl)piperazin-1-yl)cyclohexyl)morpholine (assumed) as alight yellow oil (50 mg, 9.2%). LC-MS (ES, m/z) M+1: 358/359.

Synthesis of 4-(2-((R)-4-((1S,4R)-4-((S)-3-methylmorpholino)cyclohexyl)-2-(o-tolyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide (assumed): Into an 8 mL vial were added (S)-3-methyl-4-((1R,4S)-4-((R)-3-(o-tolyl)piperazin-1-yl)cyclohexyl)morpholine (assumed) (8 mg, 0.1 mmol, 1.0 eq), 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-N-[(3R)-5-nitro-3-(oxan-4-yl)-3,4-dihydro-2H-1,4-benzoxazin-7-ylsulfonyl]-4-{2-oxo-7-azaspiro[3.5]nonan-7-yl}benzamide (18 mg, 0.1 mmol, 1.0 eq), NaBH₃CN (7 mg, 0.1 mmol, 5.0 eq), ZnCl₂ (15 mg, 0.1 mmol, 5.0 eq) and MeOH (2 mL). The resulting mixture was stirred for additional 4 hours at 70° C. The reaction was quenched with water, and then extracted with CH₂Cl₂ (5 mL). The combined organic layers were washed with brine (3×5 mL), dried over anhydrous Na₂SO₄. After filtration, the filtrate was concentrated under reduced pressure. The crude residue was purified by Prep-TLC (CH₂Cl₂/MeOH=15:1) to give 4-(2-((R)-4-((1S,4R)-4-((S)-3-methylmorpholino)cyclohexyl)-2-(o-tolyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide (assumed) as a yellow solid (10 mg, 38.6%). LC-MS (ESI, m/z) M+1: 1156. ¹H NMR (300 MHz, Chloroform-d) δ 12.61 (s, 1H), 8.41-8.39 (m, 2H), 8.31 (d, J=2.2 Hz, 1H), 8.13-8.11 (m, 1H), 7.46 (d, J=2.1 Hz, 1H), 7.13-7.11 (m, 4H), 7.09 (d, J=3.5 Hz, 1H), 6.85-6.81 (m, 1H), 6.69 (s, 1H), 6.44 (d, J=2.2 Hz, 1H), 6.13 (s, 1H), 4.55-4.40 (m, 1H), 4.16-4.02 (m, 4H), 3.98-3.93 (m, 1H), 3.66 (s, 2H), 3.50-3.46 (m, 1H), 3.44-3.41 (m, 5H), 3.38-3.32 (m, 2H), 3.09-3.08 (m, 1H), 3.05-3.00 (m, 5H), 2.70-2.68 (m, 2H), 2.38-2.35 (m, 3H), 2.04-2.00 (m, 1H), 1.78-1.76 (m, 5H), 1.71-1.69 (m, 1H), 1.47-1.40 (m, 16H), 1.28 (s, 2H), 1.20-1.15 (m, 5H), 0.95-0.90 (m, 4H), 0.89-0.85 (m, 1H).

### Example 204 Preparation of 4-(2-((R)-4-((1R,4S)-4-((S)-3-methylmorpholino)cyclohexyl)-2-(o-tolyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide (assumed)

Synthesis of 4-(2-((R)-4-((1R,4S)-4-((S)-3-methylmorpholino)cyclohexyl)-2-(o-tolyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide (assumed): Into an 8 mL vial were added (S)-3-methyl-4-((1S,4R)-4-((R)-3-(o-tolyl)piperazin-1-yl)cyclohexyl)morpholine (assumed) (8 mg, 0.1 mmol, 1.0 eq), 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-N-[(3R)-5-nitro-3-(oxan-4-yl)-3,4-dihydro-2H-1,4-benzoxazin-7-ylsulfonyl]-4-{2-oxo-7-azaspiro[3.5]nonan-7-yl}benzamide (18 mg, 0.1 mmol, 1.0 eq), NaBH₃CN (7 mg, 0.1 mmol, 5.0 eq), ZnCl₂ (15 mg, 0.1 mmol, 5.0 eq) and MeOH (1 mL). The resulting mixture was stirred for additional 4 hours at 70° C. The reaction was quenched with water, and then extracted with CH₂Cl₂ (5 mL). The combined organic layers were washed with brine (3×5 mL), dried over anhydrous Na₂SO₄. After filtration, the filtrate was concentrated under vacuum. The crude residue was purified by Prep-TLC (CH₂Cl₂/MeOH=17:1) to give 4-(2-((R)-4-((1R,4S)-4-((S)-3-methylmorpholino)cyclohexyl)-2-(o-tolyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide (assumed) as a yellow solid (10 mg, 38.7%). LC-MS (ESI, m/z) M+1: 1157. ¹H NMR (300 MHz, Chloroform-d) δ 12.61 (s, 1H), 8.41-8.37 (m, 2H), 8.31 (d, J=2.1 Hz, 1H), 8.13-8.11 (m, 1H), 7.46 (d, J=1.9 Hz, 1H), 7.13-7.12 (m, 4H), 7.10 (d, J=3.2 Hz, 1H), 6.85-6.81 (m, 1H), 6.69 (s, 1H), 6.44 (s, 1H), 6.14 (s, 1H), 4.57-4.39 (m, 2H), 4.16-3.93 (m, 5H), 3.72-3.70 (m, 4H), 3.50-3.45 (m, 1H), 3.44-3.42 (m, 7H), 3.38-3.32 (m, 2H), 3.08-3.00 (m, 6H), 2.89-2.85 (m, 5H), 2.60-2.55 (m, 3H), 2.39-2.36 (m, 3H), 2.21-2.20 (m, 2H), 1.83-1.70 (m, 2H), 1.45-1.40 (m, 4H), 1.34-1.30 (m, 1H), 1.28-1.26 (m, 2H), 1.18-1.08 (m, 8H), 1.02-1.00 (m, 3H), 0.89-0.84 m, 1H), 0.79-0.76 (m, 2H).

### Example 205 Preparation of 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-4-{2-[(2R)-4-({5-methyl-6-[(3S)-3-methylmorpholin-4-yl]pyridin-3-yl}methyl)-2-(2-methylphenyl)piperazin-1-yl]-7-azaspiro[3.5]nonan-7-yl}-N-[(3R)-5-nitro-3-(oxan-4-yl)-3,4-dihydro-2H-1,4-benzoxazin-7-ylsulfonyl]benzamide

Synthesis of 5-methyl-6-[(3S)-3-methylmorpholin-4-yl]pyridine-3-carbaldehyde: Into a 100 mL round-bottom flask, were placed 6-fluoro-5-methylpyridine-3-carbaldehyde (1.0 g, 7.2 mmol, 1.0 eq), (3S)-3-methylmorpholine (730 mg, 7.2 mmol, 1.0 eq), DIEA (2.8 g, 21.6 mmol, 3.0 eq), DMSO (10 mL). The resulting solution was stirred for 16 hours at 130° C. in an oil bath. The resulting mixture was then quenched by the addition of water (100 mL), and then extracted with ethyl acetate (2×100 mL). The combined organic layer was washed with water (2×100 mL) and brine (2×100 mL), dried over anhydrous Na₂SO₄. After filtration, the filtrate was concentrated under vacuum. The crude residue was applied onto a silica gel column and eluted with ethyl acetate/petroleum ether=1:3 to give 5-methyl-6-[(3S)-3-methylmorpholin-4-yl]pyridine-3-carbaldehyde as a yellow solid (1.0 g, 63.2%). LC-MS (ES, m/z) M+1: 221.

Synthesis of (3S)-3-methyl-4-(3-methyl-5-{[(3R)-3-(2-methylphenyl)piperazin-1-yl]methyl}pyridin-2-yl)morpholine: Into an 8-mL sealed tube, were placed 5-methyl-6-[(3S)-3-methylmorpholin-4-yl]pyridine-3-carbaldehyde (120 mg, 0.5 mmol, 1.0 eq), (2R)-2-(2-methylphenyl)piperazine (115 mg, 0.7 mmol, 1.2 eq), ZnCl₂ (223 mg, 1.6 mmol, 3.0 eq), NaBH₃CN (103 mg, 1.6 mmol, 3.0 eq), MeOH (1 mL). The resulting solution was stirred for 1.5 hours at 25° C. The resulting mixture was then quenched by the addition of water (30 mL), and then extracted with dichloromethane (2×30 mL). The combined organic layers was washed with brine (2×30 mL), and dried over anhydrous Na₂SO₄. After filtration, the filtrate was concentrated under vacuum. The crude residue was applied onto a silica gel column and eluted with dichloromethane/methanol=10:1 to give (3S)-3-methyl-4-(3-methyl-5-{[(3R)-3-(2-methylphenyl)piperazin-1-yl]methyl}pyridin-2-yl)morpholine as a light yellow oil (100 mg, 48.2%). LC-MS (ES, m/z) M+1: 381.

Synthesis of 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-4-{2-[(2R)-4-({5-methyl-6-[(3S)-3-methylmorpholin-4-yl]pyridin-3-yl}methyl)-2-(2-methylphenyl)piperazin-1-yl]-7-azaspiro[3.5]nonan-7-yl}-N-[(3R)-5-nitro-3-(oxan-4-yl)-3,4-dihydro-2H-1,4-benzoxazin-7-ylsulfonyl]benzamide: Into an 8-mL sealed tube, were placed 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-N-[(3R)-5-nitro-3-(oxan-4-yl)-3,4-dihydro-2H-1,4-benzoxazin-7-ylsulfonyl]-4-{2-oxo-7-azaspiro[3.5]nonan-7-yl}benzamide (20 mg, 0.02 mmol, 1.0 eq), (3S)-3-methyl-4-(3-methyl-5-{[(3R)-3-(2-methylphenyl)piperazin-1-yl]methyl}pyridin-2-yl)morpholine (11 mg, 0.03 mmol, 1.2 eq), ZnCl₂ (10 mg, 0.08 mmol, 3.0 eq), NaBH₃CN (5 mg, 0.08 mmol, 3.0 eq), MeOH (0.5 mL). The resulting solution was stirred for 4 hours at 70° C. in an oil bath. The resulting mixture was then quenched by the addition of water (30 mL), and then extracted with dichloromethane (2×30 mL). The combined organic layers was washed with brine (2×30 mL), dried over anhydrous Na₂SO₄. After filtration, the filtrate was concentrated under vacuum. The crude residue was applied onto a silica gel column and eluted with dichloromethane/methanol=15:1 to give 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-4-{2-[(2R)-4-({5-methyl-6-[(3S)-3-methylmorpholin-4-yl]pyridin-3-yl}methyl)-2-(2-methylphenyl)piperazin-1-yl]-7-azaspiro[3.5]nonan-7-yl}-N-[(3R)-5-nitro-3-(oxan-4-yl)-3,4-dihydro-2H-1,4-benzoxazin-7-ylsulfonyl]benzamide as a yellow solid (8 mg, 27.6%). LC-MS (ES, m/z) M+1: 1179. ¹H NMR (400 MHz, Methanol-d₄) δ 8.19-8.09 (m, 2H), 8.05-7.99 (m, 1H), 7.91-7.84 (m, 1H), 7.64-7.57 (m, 1H), 7.48-7.42 (m, 1H), 7.24-7.13 (m, 3H), 7.06-7.00 (m, 1H), 6.98-6.89 (m, 1H), 6.72-6.66 (m, 1H), 6.51-6.45 (m, 1H), 6.03-5.95 (m, 1H), 4.61-4.56 (m, 1H), 4.34-4.21 (m, 2H), 4.15-4.08 (m, 1H), 4.03-3.96 (m, 2H), 3.95-3.78 (m, 2H), 3.68-3.38 (m, 6H), 3.21-3.04 (m, 6H), 3.01-2.68 (m, 2H), 2.44-2.24 (m, 12H), 1.92-1.83 (m, 2H), 1.82-1.65 (m, 4H), 1.63-1.37 (m, 2H), 1.33-1.29 (m, 6H), 1.19-1.12 (m, 1H), 0.95-0.81 (m, 9H).

### Example 206 Preparation of 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-4-{2-[(2R)-4-({6-[(3S)-3-methylmorpholin-4-yl]-5-(trifluoromethyl)pyridin-3-yl}methyl)-2-(2-methylphenyl)piperazin-1-yl]-7-azaspiro[3.5]nonan-7-yl}-N-[(3R)-5-nitro-3-(oxan-4-yl)-3,4-dihydro-2H-1,4-benzoxazin-7-ylsulfonyl]benzamide

Synthesis of (3S)-4-[5-bromo-3-(trifluoromethyl)pyridin-2-yl]-3-methylmorpholine: Into a 100-mL round-bottom flask, were placed 5-bromo-2-chloro-3-(trifluoromethyl)pyridine (6.0 g, 23.0 mmol, 1.0 eq), (3S)-3-methylmorpholine (3.5 g, 34.6 mmol, 1.5 eq), DIEA (8.9 g, 69.1 mmol, 3.0 eq), DMSO (18 mL). The resulting solution was stirred for 16 hours at 130° C. The resulting mixture was then quenched by the addition of water (300 mL), and then extracted with ethyl acetate (2×300 mL). The combined organic layers was washed with brine (3×300 mL), dried over anhydrous Na₂SO₄. After filtration, the filtrate was concentrated under vacuum. The crude residue was purified onto a silica gel column and eluted with ethyl acetate/petroleum ether=1:1 to give (3S)-4-[5-bromo-3-(trifluoromethyl)pyridin-2-yl]-3-methylmorpholine as a light yellow oil (2.7 g, 36.1%). ¹H NMR (300 MHz, DMSO-d₆) δ 8.80 (d, J=2.4 Hz, 1H), 8.35 (d, J=2.4 Hz, 1H), 3.81-3.69 (m, 2H), 3.68-3.47 (m, 2H), 3.34-3.24 (m, 1H), 3.03-2.94 (m, 1H), 2.91-2.85 (m, 1H), 0.76 (d, J=6.3 Hz, 3H).

Synthesis of 6-[(3S)-3-methylmorpholin-4-yl]-5-(trifluoromethyl)pyridine-3-carbaldehyde: Into a 50-mL 3-necked round-bottom flask, were placed (3S)-4-[5-bromo-3-(trifluoromethyl)pyridin-2-yl]-3-methylmorpholine (1.0 g, 3.1 mmol, 1.0 eq), THE (10 mL). This was followed by the addition of n-BuLi (2.5 M in hexanes, 2 mL) at −78° C. Then the mixture was stirred at −78° C. for 30 minutes. After that, DMF (300 mg, 4.3 mmol, 1.4 eq) was added at −78° C. The mixture was stirred at −78° C. for an additional 2 hours, which was then quenched by the addition of NH₄Cl (aq.) (100 mL), and then extracted with ethyl acetate (2×100 mL). The combined organic layers was washed with brine (2×100 mL), dried over anhydrous Na₂SO₄. After filtration, the filtrate was concentrated under vacuum. The crude residue was purified onto a silica gel column and eluted with ethyl acetate/petroleum ether=1:3 to give 6-[(3S)-3-methylmorpholin-4-yl]-5-(trifluoromethyl)pyridine-3-carbaldehyde as a light yellow oil (350 mg, 41.5%). ¹H NMR (300 MHz, DMSO-d₆) δ 9.96 (s, 1H), 8.92 (d, J=2.1 Hz, 1H), 8.40 (d, J=2.1 Hz, 1H), 4.29-4.22 (m, 1H), 3.87-3.82 (m, 1H), 3.72-3.62 (m, 2H), 3.61-3.48 (m, 1H), 3.46-3.38 (m, 2H), 1.18 (d, J=6.6 Hz, 3H).

Synthesis of (3S)-3-methyl-4-(5-{[(3R)-3-(2-methylphenyl)piperazin-1-yl]methyl}-3-(trifluoromethyl)pyridin-2-yl)morpholine: Into a 20-mL sealed-tube, were placed (2R)-2-(2-methylphenyl)piperazine (100 mg, 0.6 mmol, 1.0 eq), 6-[(3S)-3-methylmorpholin-4-yl]-5-(trifluoromethyl)pyridine-3-carbaldehyde (140 mg, 0.5 mmol, 0.9 eq), MeOH (2 mL), ZnCl₂ (232 mg, 1.7 mmol, 3.0 eq), NaBH₃CN (107 mg, 1.7 mmol, 3.0 eq). The resulting solution was stirred for 4 hours at 25° C. The resulting mixture was then quenched by the addition of water (50 mL), and then extracted with dichloromethane/methanol=20:1 (2×50 mL). The combined organic layers was washed with brine (2×50 mL) and dried over anhydrous Na₂SO₄. After filtration, the filtrate was concentrated under vacuum. The crude residue was purified onto a silica gel column and eluted with dichloromethane/methanol=15:1 to give (3S)-3-methyl-4-(5-{[(3R)-3-(2-methylphenyl)piperazin-1-yl]methyl}-3-(trifluoromethyl)pyridin-2-yl)morpholine as a light yellow oil (90 mg, 36.5%). LC-MS (ES, m/z) M+1: 435.

Synthesis of 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-4-{2-[(2R)-4-({6-[(3S)-3-methylmorpholin-4-yl]-5-(trifluoromethyl)pyridin-3-yl}methyl)-2-(2-methylphenyl)piperazin-1-yl]-7-azaspiro[3.5]nonan-7-yl}-N-[(3R)-5-nitro-3-(oxan-4-yl)-3,4-dihydro-2H-1,4-benzoxazin-7-ylsulfonyl]benzamide: Into an 8-mL sealed-tube, were placed 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-N-[(3R)-5-nitro-3-(oxan-4-yl)-3,4-dihydro-2H-1,4-benzoxazin-7-ylsulfonyl]-4-{2-oxo-7-azaspiro[3.5]nonan-7-yl}benzamide (20 mg, 0.03 mmol, 1.0 eq), (3S)-3-methyl-4-(5-{[(3R)-3-(2-methylphenyl)piperazin-1-yl]methyl}-3-(trifluoromethyl)pyridin-2-yl)morpholine (14 mg, 0.03 mmol, 1.3 eq), MeOH (1 mL), ZnCl₂ (10 mg, 0.08 mmol, 3.0 eq), NaBH₃CN (5 mg, 0.08 mmol, 3.0 eq). The resulting solution was stirred for 4 hours at 70° C. The resulting mixture was then quenched by the addition of water (20 mL), and then extracted with dichloromethane/methanol=10:1 (2×30 mL). The combined organic layers was washed with brine (2×20 mL) and dried over anhydrous Na₂SO₄. After filtration, the filtrate was concentrated under vacuum. The residue was purified by Prep-TLC (dichloromethane/methanol=15:1) to give 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-4-{2-[(2R)-4-({6-[(3S)-3-methylmorpholin-4-yl]-5-(trifluoromethyl)pyridin-3-yl}methyl)-2-(2-methylphenyl)piperazin-1-yl]-7-azaspiro[3.5]nonan-7-yl}-N-[(3R)-5-nitro-3-(oxan-4-yl)-3,4-dihydro-2H-1,4-benzoxazin-7-ylsulfonyl]benzamide as a yellow solid (8 mg, 26.4%). LC-MS (ES, m/z) M+1: 1233. ¹H NMR (400 MHz, Methanol-d₄) δ 8.56 (d, J=2.0 Hz, 1H), 8.17-8.06 (m, 2H), 8.02 (d, J=2.0 Hz, 1H), 7.87 (d, J=9.0 Hz, 1H), 7.48-7.44 (m, 1H), 7.21 (d, J=2.2 Hz, 1H), 7.15-7.13 (m, 2H), 7.03 (d, J=3.6 Hz, 1H), 6.99-6.89 (m, 1H), 6.70-6.69 (m, 1H), 6.49 (s, 1H), 5.99 (d, J=3.4 Hz, 1H), 4.33-4.27 (m, 2H), 4.26-4.22 (m, 1H), 4.14-4.10 (m, 1H), 4.03-3.90 (m, 3H), 3.86-3.80 (m, 2H), 3.78-3.60 (m, 6H), 3.53-3.44 (m, 4H), 3.43-3.38 (m, 3H), 3.18-3.06 (m, 6H), 2.99-2.91 (m, 3H), 2.71-2.66 (m, 1H), 2.42-2.33 (m, 5H), 1.91-1.83 (m, 2H), 1.77-1.66 (m, 4H), 1.60-1.40 (m, 6H), 1.36-1.29 (m, 6H).

### Example 207 Preparation of 4-(2-((R)-4-((1r,4R)-4-hydroxy-4-methylcyclohexyl)-2-(2-isopropoxyphenyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide (assumed)

Synthesis of (1R,4r)-4-((R)-3-(2-isopropoxyphenyl)piperazin-1-yl)-1-methylcyclohexan-1-ol (assumed) and (1S,4s)-4-((R)-3-(2-isopropoxyphenyl)piperazin-1-yl)-1-methylcyclohexan-1-ol (assumed): Into a 40 mL vial were added (2R)-2-(2-isopropoxyphenyl)piperazine (500 mg, 2.2 mmol, 1.0 eq), 4-hydroxy-4-methylcyclohexan-1-one (290 mg, 2.2 mmol, 1.0 eq), NaBH₃CN (713 mg, 11.3 mmol, 5.0 eq), ZnCl₂(1546 mg, 11.3 mmol, 5.0 eq) and MeOH (6 mL). The resulting mixture was stirred for additional 4 hours at 70° C. The reaction was quenched with water, and then extracted with CH₂Cl₂ (5 mL). The combined organic layers were washed with brine (3×5 mL), dried over anhydrous Na₂SO₄. After filtration, the filtrate was concentrated under vacuum. The crude residue was purified by Prep-TLC (CH₂Cl₂/MeOH=8:1) to give (1r,4r)-4-[(3R)-3-(2-isopropoxyphenyl)piperazin-1-yl]-1-methylcyclohexan-1-ol (assumed) as a light yellow solid (50 mg, 6.6%) and (1s,4s)-4-[(3R)-3-(2-isopropoxyphenyl)piperazin-1-yl]-1-methylcyclohexan-1-ol (assumed) as a light yellow solid (46 mg, 6.0%). LC-MS (ES, m/z) M+1: 333.

Synthesis of 4-(2-((R)-4-((1r,4R)-4-hydroxy-4-methylcyclohexyl)-2-(2-isopropoxyphenyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide (assumed): Into an 8 mL vial were added (1r,4r)-4-[(3R)-3-(2-isopropoxyphenyl)piperazin-1-yl]-1-methylcyclohexan-1-ol (assumed) (8 mg, 0.1 mmol, 1.0 eq), 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-N-[(3R)-5-nitro-3-(oxan-4-yl)-3,4-dihydro-2H-1,4-benzoxazin-7-ylsulfonyl]-4-{2-oxo-7-azaspiro[3.5]nonan-7-yl}benzamide (19 mg, 0.1 mmol, 1.0 eq), NaBH₃CN (7 mg, 0.1 mmol, 5.0 eq), ZnCl₂ (16 mg, 0.1 mmol, 5.0 eq) and MeOH (1 mL). The resulting mixture was stirred for additional 4 hours at 70° C. The reaction was quenched with water, and then extracted with CH₂Cl₂ (5 mL). The combined organic layers were washed with brine (3×5 mL), dried over anhydrous Na₂SO₄. After filtration, the filtrate was concentrated under vacuum. The crude residue was purified by Prep-TLC (CH₂Cl₂/MeOH=17:1) to give 4-(2-((R)-4-((1r,4R)-4-hydroxy-4-methylcyclohexyl)-2-(2-isopropoxyphenyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide (assumed) as a yellow solid (10 mg, 36.7%). LC-MS (ESI, m/z) M+1: 1131. ¹H NMR (300 MHz, Chloroform-d) δ 12.63 (s, 1H), 8.45 (s, 1H), 8.34-8.32 (m, 2H), 8.13-8.11 (m, 1H), 7.46 (s, 1H), 7.09 (s, 1H), 6.86 (s, 2H), 6.69 (s, 1H), 6.43 (s, 1H), 6.13 (s, 1H), 4.63-4.58 (m, 3H), 4.53 (s, 1H), 4.45-4.40 (m, 1H), 4.13 (s, 1H), 4.12 (s, 1H), 4.02-3.96 (m, 3H), 3.61 (s, 1H), 3.44-3.31 (m, 9H), 3.07-2.93 (m, 8H), 2.43 (s, 1H), 2.24 (s, 2H), 1.85-1.80 (m, 8H), 1.47-1.43 (m, 7H), 1.36-1.30 (m, 8H), 1.26-1.24 (m, 5H), 0.89-0.85 (m, 2H), 0.78-0.75 (m, 2H).

### Example 208 Preparation of 4-(2-((R)-2-(2-isopropoxyphenyl)-4-((S)-tetrahydro-2H-pyran-3-yl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide (assumed)

Synthesis of 4-(2-((R)-2-(2-isopropoxyphenyl)-4-((S)-tetrahydro-2H-pyran-3-yl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide (assumed): Into an 8 mL vial were added (1s,4s)-4-[(3R)-3-(2-isopropoxyphenyl)piperazin-1-yl]-1-methylcyclohexan-1-ol (assumed) (8 mg, 0.1 mmol, 1.0 eq), 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-N-[(3R)-5-nitro-3-(oxan-4-yl)-3,4-dihydro-2H-1,4-benzoxazin-7-ylsulfonyl]-4-{2-oxo-7-azaspiro[3.5]nonan-7-yl}benzamide (19 mg, 0.1 mmol, 1.0 eq), NaBH₃CN (7 mg, 0.1 mmol, 5.0 eq), ZnCl₂ (16 mg, 0.1 mmol, 5.0 eq) and MeOH (1 mL). The resulting mixture was stirred for additional 4 hours at 70° C. The reaction was quenched with water, and then extracted with CH₂Cl₂ (5 mL). The combined organic layers were washed with brine (3×5 mL), dried over anhydrous Na₂SO₄. After filtration, the filtrate was concentrated under vacuum. The crude residue was purified by Prep-TLC (CH₂Cl₂/MeOH=17:1) to give 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-4-{2-[(2R)-2-(2-isopropoxyphenyl)-4-[(1s,4s)-4-hydroxy-4-methylcyclohexyl]piperazin-1-yl]-7-azaspiro[3.5]nonan-7-yl}-N-[(3R)-5-nitro-3-(oxan-4-yl)-3,4-dihydro-2H-1,4-benzoxazin-7-ylsulfonyl]benzamide (assumed) as a yellow solid (10 mg, 36.7%). LC-MS (ESI, m/z) M+1: 1132. ¹H NMR (300 MHz, Chloroform-d) δ 12.64 (s, 1H), 8.45 (s, 2H), 8.31-8.27 (m, 1H), 8.12-8.10 (m, 1H), 7.46 (d, J=2.1 Hz, 1H), 7.17 (s, 1H), 7.13-7.05 (m, 1H), 6.84-6.81 (m, 4H), 6.69 (s, 1H), 6.44 (s, 1H), 6.13-6.09 (m, 1H), 4.57-4.39 (m, 1H), 4.12-4.11 (m, 2H), 4.10-4.02 (m, 2H), 3.97-3.93 (m, 1H), 3.44-3.39 (m, 8H), 3.08-3.00 (m, 1H), 2.78 (s, 1H), 2.52 (s, 1H), 2.27-2.19 (m, 3H), 1.85-1.79 (m, 8H), 1.47-1.39 (m, 15H), 1.34-1.32 (m, 7H), 1.22-1.17 (m, 4H), 0.88-0.82 (m, 2H), 0.76-0.73 (m, 2H).

### Example 209 Preparation of 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-4-{2-[(2R)-2-(2-isopropoxyphenyl)-4-{3-oxaspiro[5.5]undecan-9-yl}piperazin-1-yl]-7-azaspiro[3.5]nonan-7-yl}-N-[(3R)-5-nitro-3-(oxan-4-yl)-3,4-dihydro-2H-1,4-benzoxazin-7-ylsulfonyl]benzamide

Synthesis of (3R)-3-(2-isopropoxyphenyl)-1-{3-oxaspiro[5.5]undecan-9-yl}piperazine: Into an 8-mL sealed tube, were placed 3-oxaspiro[5.5]undecan-9-one (100 mg, 0.6 mmol, 1.0 eq), (2R)-2-(2-isopropoxyphenyl)piperazine (157 mg, 0.7 mmol, 1.2 eq), ZnCl₂ (243 mg, 1.8 mmol, 3.0 eq), NaBH₃CN (112 mg, 1.8 mmol, 3.0 eq), MeOH (1 mL). The resulting solution was stirred for 4 hours at 25° C. The resulting mixture was then quenched by the addition of water (30 mL), and then extracted with dichloromethane (2×30 mL). The combined organic layers was washed with brine (2×30 mL) and dried over anhydrous Na₂SO₄. After filtration, the filtrate was concentrated under vacuum. The crude residue was applied onto a silica gel column and eluted with dichloromethane/methanol=10:1 to give (3R)-3-(2-isopropoxyphenyl)-1-{3-oxaspiro[5.5]undecan-9-yl}piperazine as a light yellow oil (100 mg, 45.2%). LC-MS (ESI, m/z) M+1: 373.

Synthesis of 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-4-{2-[(2R)-2-(2-isopropoxyphenyl)-4-{3-oxaspiro[5.5]undecan-9-yl}piperazin-1-yl]-7-azaspiro[3.5]nonan-7-yl}-N-[(3R)-5-nitro-3-(oxan-4-yl)-3,4-dihydro-2H-1,4-benzoxazin-7-ylsulfonyl]benzamide: Into an 8-mL sealed tube, were placed 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-N-[(3R)-5-nitro-3-(oxan-4-yl)-3,4-dihydro-2H-1,4-benzoxazin-7-ylsulfonyl]-4-{2-oxo-7-azaspiro[3.5]nonan-7-yl}benzamide (20 mg, 0.03 mmol, 1.0 eq), (3R)-3-(2-isopropoxyphenyl)-1-{3-oxaspiro[5.5]undecan-9-yl}piperazine (11 mg, 0.03 mmol, 1.2 eq), ZnCl₂ (10 mg, 0.08 mmol, 3.0 eq), NaBH₃CN (5 mg, 0.08 mmol, 3.0 eq), MeOH (0.5 mL). The resulting solution was stirred for 4 hours at 70° C. in an oil bath. The crude product was purified by Flash-Prep-HPLC using the following conditions: Column: XBridge Shield RP18 OBD Column, 30*150 mm, 5 μm; Mobile Phase A: water (0.05% NH₃·H₂O), Mobile Phase B: CH₃CN; Flow rate: 60 mL/min; Gradient: 20% B to 50% B in 8 min, 50% B; Wave Length: 254/220 nm; RT1(min): 6.83; Number Of Runs: 2. Finally, 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-4-{2-[(2R)-2-(2-isopropoxyphenyl)-4-{3-oxaspiro[5.5]undecan-9-yl}piperazin-1-yl]-7-azaspiro[3.5]nonan-7-yl}-N-[(3R)-5-nitro-3-(oxan-4-yl)-3,4-dihydro-2H-1,4-benzoxazin-7-ylsulfonyl]benzamide was obtained as a yellow solid (6 mg, 20.9%). LC-MS (ESI, m/z) M+1: 1171. ¹H NMR (400 MHz, Methanol-d₄) δ 8.27-8.02 (m, 1H), 7.84-7.72 (m, 1H), 7.45-7.20 (m, 3H), 7.02-6.87 (m, 4H), 6.77-6.50 (m, 2H), 6.07-5.95 (m, 1H), 4.27-3.95 (m, 6H), 3.93-3.76 (m, 1H), 3.70-3.59 (m, 4H), 3.51-3.39 (m, 4H), 3.23-3.04 (m, 8H), 2.93-2.85 (m, 1H), 2.67-2.21 (m, 4H), 1.94-1.58 (m, 12H), 1.57-1.40 (m, 10H), 1.38-1.29 (m, 9H), 1.24-1.11 (m, 4H), 0.95-0.87 (m, 1H).

### Example 210 Preparation of 4-{2-[(2R)-4-[(4S)-6,7-difluoro-3,4-dihydro-2H-1-benzopyran-4-yl]-2-(2-isopropoxyphenyl)piperazin-1-yl]-7-azaspiro[3.5]nonan-7-yl}-2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-N-[(3R)-5-nitro-3-(oxan-4-yl)-3,4-dihydro-2H-1,4-benzoxazin-7-ylsulfonyl]benzamide (assumed)

Synthesis of (3R)-1-[(4S)-6,7-difluoro-3,4-dihydro-2H-1-benzopyran-4-yl]-3-(2-isopropoxyphenyl)piperazine (assumed) and (3R)-1-[(4R)-6,7-difluoro-3,4-dihydro-2H-1-benzopyran-4-yl]-3-(2-isopropoxyphenyl)piperazine (assumed): Into a 40 mL vial were added 4-chloro-6,7-difluoro-3,4-dihydro-2H-1-benzopyran (400 mg, 2.0 mmol, 1.0 eq), (2R)-2-(2-isopropoxyphenyl)piperazine (assumed) (431 mg, 2.0 mmol, 1.0 eq), K₂CO₃ (811 mg, 5.9 mmol, 3.0 eq) and CH₃CN (5 mL). The resulting mixture was stirred for overnight at 80° C. The reaction was quenched by the addition of water (5 mL) and extracted with EtOAc (3×10 mL). The combined organic layers were washed with brine (10 mL), dried over anhydrous Na₂SO₄. After filtration, the filtrate was concentrated under vacuum. The residue was purified by Prep-TLC (EtOAc) to give (3R)-1-[(4S)-6,7-difluoro-3,4-dihydro-2H-1-benzopyran-4-yl]-3-(2-isopropoxyphenyl)piperazine (assumed) (80 mg, 10.5%) and (3R)-1-[(4R)-6,7-difluoro-3,4-dihydro-2H-1-benzopyran-4-yl]-3-(2-isopropoxyphenyl)piperazine (assumed) as a colorless solid (110 mg, 14.5%). ¹H NMR_A (300 MHz, Chloroform-d) δ 7.58-7.36 (m, 2H), 7.23 (td, J=7.8, 1.8 Hz, 1H), 7.05-6.84 (m, 2H), 6.60 (dd, J=11.5, 6.9 Hz, 1H), 4.61 (hept, J=6.1 Hz, 1H), 4.43-4.28 (m, 2H), 4.23-4.03 (m, 1H), 3.82 (dd, J=9.0, 6.1 Hz, 1H), 3.09 (dt, J=11.3, 2.8 Hz, 1H), 3.04-2.90 (m, 2H), 2.70-2.53 (m, 2H), 2.35 (td, J=10.9, 3.1 Hz, 2H), 2.19-2.04 (m, 1H), 1.93 (dtd, J=13.5, 5.4, 2.5 Hz, 1H), 1.38 (dd, J=9.2, 6.0 Hz, 6H). ¹H NMR_B (300 MHz, Chloroform-d) δ 7.61-7.45 (m, 2H), 7.18 (ddd, J=8.2, 7.4, 1.8 Hz, 1H), 6.90 (td, J=7.5, 1.1 Hz, 1H), 6.88-6.78 (m, 1H), 6.57 (dd, J=11.5, 6.9 Hz, 1H), 4.52 (hept, J=6.1 Hz, 1H), 4.31 (dt, J=11.1, 4.2 Hz, 1H), 4.19 (dd, J=9.5, 2.6 Hz, 1H), 4.09 (ddd, J=11.0, 10.0, 3.0 Hz, 1H), 3.89 (t, J=7.8 Hz, 1H), 3.28-3.09 (m, 2H), 2.94-2.70 (m, 3H), 2.19-1.89 (m, 4H), 1.35 (d, J=6.0 Hz, 3H), 1.17 (d, J=6.1 Hz, 3H).

Synthesis of 4-{2-[(2R)-4-[(4S)-6,7-difluoro-3,4-dihydro-2H-1-benzopyran-4-yl]-2-(2-isopropoxyphenyl)piperazin-1-yl]-7-azaspiro[3.5]nonan-7-yl}-2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-N-[(3R)-5-nitro-3-(oxan-4-yl)-3,4-dihydro-2H-1,4-benzoxazin-7-ylsulfonyl]benzamide (assumed): Into an 8 mL vial were added (3R)-1-[(4S)-6,7-difluoro-3,4-dihydro-2H-1-benzopyran-4-yl]-3-(2-isopropoxyphenyl)piperazine (assumed) (20 mg, 0.05 mmol, 1.0 eq), 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-N-[(3R)-5-nitro-3-(oxan-4-yl)-3,4-dihydro-2H-1,4-benzoxazin-7-ylsulfonyl]-4-{2-oxo-7-azaspiro[3.5]nonan-7-yl}benzamide (42 mg, 0.05 mmol, 1.0 eq), NaBH₃CN (16 mg, 0.3 mmol, 5.0 eq), ZnCl₂ (35 mg, 0.23 mmol, 5 eq) and MeOH (3 mL). The resulting mixture was stirred for overnight at 70° C. The reaction was quenched by the addition of water (2 mL) and extracted with CH₂Cl₂ (3×5 mL). The combined organic layers were washed with brine (3 mL), dried over anhydrous Na₂SO₄. After filtration, the filtrate was concentrated under vacuum. The residue was purified by reversed-phase flash chromatography using the following conditions: column, C18 silica gel; mobile phase, MeCN in water (0.1% FA), 10% to 50% gradient in 10 min; detector, UV 254 nm. Finally, 4-{2-[(2R)-4-[(4S)-6,7-difluoro-3,4-dihydro-2H-1-benzopyran-4-yl]-2-(2-isopropoxyphenyl)piperazin-1-yl]-7-azaspiro[3.5]nonan-7-yl}-2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-N-[(3R)-5-nitro-3-(oxan-4-yl)-3,4-dihydro-2H-1,4-benzoxazin-7-ylsulfonyl]benzamide was obtained (assumed) as a yellow solid (12 mg, 19.6%). LC-MS (ES, m/z) M+1: 1187. ¹H NMR (300 MHz, Chloroform-d) δ 12.63 (s, 1H), 8.45 (s, 2H), 8.31 (d, J=2.1 Hz, 1H), 8.13 (d, J=9.2 Hz, 1H), 7.46 (d, J=2.3 Hz, 1H), 7.10 (d, J=2.9 Hz, 2H), 6.85 (d, J=7.8 Hz, 3H), 6.69 (s, 2H), 6.43 (s, 1H), 6.13 (d, J=3.6 Hz, 1H), 4.45-4.42 (m, 3H), 4.17-3.91 (m, 6H), 3.62-3.32 (m, 7H), 3.07-2.96 (m, 6H), 2.06-2.01 (m, 6H), 1.71-1.62 (m, 12H), 1.42-1.36 (m, 12H).

### Example 211 Preparation of 4-{2-[(2R)-4-[(4R)-6,7-difluoro-3,4-dihydro-2H-1-benzopyran-4-yl]-2-(2-isopropoxyphenyl)piperazin-1-yl]-7-azaspiro[3.5]nonan-7-yl}-2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-N-[(3R)-5-nitro-3-(oxan-4-yl)-3,4-dihydro-2H-1,4-benzoxazin-7-ylsulfonyl]benzamide (assumed)

Synthesis of 4-{2-[(2R)-4-[(4R)-6,7-difluoro-3,4-dihydro-2H-1-benzopyran-4-yl]-2-(2-isopropoxyphenyl)piperazin-1-yl]-7-azaspiro[3.5]nonan-7-yl}-2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-N-[(3R)-5-nitro-3-(oxan-4-yl)-3,4-dihydro-2H-1,4-benzoxazin-7-ylsulfonyl]benzamide (assumed): Into an 8 mL vial were added (3R)-1-[(4R)-6,7-difluoro-3,4-dihydro-2H-1-benzopyran-4-yl]-3-(2-isopropoxyphenyl)piperazine (assumed) (20 mg, 0.05 mmol, 1.0 eq), 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-N-[(3R)-5-nitro-3-(oxan-4-yl)-3,4-dihydro-2H-1,4-benzoxazin-7-ylsulfonyl]-4-{2-oxo-7-azaspiro[3.5]nonan-7-yl}benzamide (42 mg, 0.05 mmol, 1.0 eq), NaBH₃CN (16 mg, 0.3 mmol, 5.0 eq), ZnCl₂ (35 mg, 0.3 mmol, 5.0 eq) and MeOH (3 mL). The resulting mixture was stirred for overnight at 70° C. The reaction was quenched by the addition of water (2 mL) and extracted with CH₂Cl₂ (3×5 mL). The combined organic layers were washed with brine (5 mL), dried over anhydrous Na₂SO₄. After filtration, the filtrate was concentrated under vacuum. The residue was purified by reversed-phase flash chromatography using the following conditions: column, C18 silica gel; mobile phase, MeCN in water (0.1% FA), 10% to 50% gradient in 10 min; detector, UV 254 nm. Finally, 4-{2-[(2R)-4-[(4R)-6,7-difluoro-3,4-dihydro-2H-1-benzopyran-4-yl]-2-(2-isopropoxyphenyl)piperazin-1-yl]-7-azaspiro[3.5]nonan-7-yl}-2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-N-[(3R)-5-nitro-3-(oxan-4-yl)-3,4-dihydro-2H-1,4-benzoxazin-7-ylsulfonyl]benzamide (assumed) was obtained as a yellow solid (8 mg, 8.0%). LC-MS (ES, m/z) M+1: 1187. ¹H NMR (300 MHz, Chloroform-d) δ 12.64 (s, 1H), 8.46 (s, 1H), 8.43 (s, 1H), 8.31 (d, J=2.1 Hz, 2H), 8.13 (d, J=9.1 Hz, 1H), 7.46 (d, J=2.1 Hz, 2H), 7.10 (t, J=3.0 Hz, 1H), 6.86 (d, J=9.5 Hz, 3H), 6.69 (s, 1H), 6.55 (s, 1H), 6.44 (s, 1H), 6.14 (dd, J=3.5, 2.0 Hz, 1H), 4.71-4.40 (m, 4H), 4.40-4.20 (m, 2H), 4.20-3.76 (m, 8H), 3.45-3.24 (m, 7H), 2.99-2.84 (m, 8H), 2.38-2.21 (m, 4H), 2.06-1.95 (m, 4H), 1.91-1.34 (m, 8H), 1.34-1.02 (m, 6H).

### Example 212 Preparation of 4-{2-[(2R)-4-[(4R)-7,8-difluoro-3,4-dihydro-2H-1-benzopyran-4-yl]-2-(2-isopropoxyphenyl)piperazin-1-yl]-7-azaspiro[3.5]nonan-7-yl}-2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-N-[(3R)-5-nitro-3-(oxan-4-yl)-3,4-dihydro-2H-1,4-benzoxazin-7-ylsulfonyl]benzamide (assumed)

Synthesis of (3R)-1-[(4S)-7,8-difluoro-3,4-dihydro-2H-1-benzopyran-4-yl]-3-(2-isopropoxyphenyl)piperazine (assumed) and (3R)-1-[(4R)-7,8-difluoro-3,4-dihydro-2H-1-benzopyran-4-yl]-3-(2-isopropoxyphenyl)piperazine (assumed): Into a 40 mL vial were added 4-chloro-7,8-difluoro-3,4-dihydro-2H-1-benzopyran (500 mg, 2.4 mmol, 1 eq), K₂CO₃ (1013 mg, 7.3 mmol, 3.0 eq) and CH₃CN (5 mL). The resulting mixture was stirred for overnight at 80° C. The reaction was quenched by the addition of water (5 mL) and extracted with EtOAc (3×10 mL). The combined organic layers were washed with brine (3×10 mL), dried over anhydrous Na₂SO₄. After filtration, the filtrate was concentrated under vacuum. The residue was purified by Prep-TLC (EtOAc) to give (3R)-1-[(4S)-7,8-difluoro-3,4-dihydro-2H-1-benzopyran-4-yl]-3-(2-isopropoxyphenyl)piperazine (assumed) (110 mg, 11.6%) and (3R)-1-[(4R)-7,8-difluoro-3,4-dihydro-2H-1-benzopyran-4-yl]-3-(2-isopropoxyphenyl)piperazine (assumed) as a colorless solid (160 mg, 16.9%). ¹H NMR_A (300 MHz, Chloroform-d) δ 7.48 (dd, J=7.6, 1.8 Hz, 1H), 7.27-7.17 (m, 2H), 6.94 (td, J=7.5, 1.2 Hz, 1H), 6.91-6.84 (m, 1H), 6.69 (ddd, J=9.9, 8.9, 7.1 Hz, 1H), 4.66-4.55 (m, 1H), 4.52 (ddd, J=9.6, 4.9, 2.4 Hz, 1H), 4.33-4.19 (m, 2H), 3.88-3.74 (m, 1H), 3.09 (dt, J=11.3, 2.8 Hz, 1H), 3.03-2.86 (m, 2H), 2.64 (dq, J=10.8, 2.4 Hz, 1H), 2.52 (t, J=10.1 Hz, 1H), 2.34 (td, J=10.9, 3.1 Hz, 1H), 2.17 (dtd, J=13.8, 8.9, 3.5 Hz, 2H), 1.96 (dtd, J=13.9, 5.7, 2.8 Hz, 1H), 1.35 (dd, J=16.6, 6.0 Hz, 6H). ¹H NMR_B (300 MHz, Chloroform-d) δ 7.55-7.46 (m, 1H), 7.35 (ddt, J=8.9, 3.6, 1.8 Hz, 1H), 7.18 (ddd, J=8.2, 7.4, 1.8 Hz, 1H), 6.90 (td, J=7.5, 1.1 Hz, 1H), 6.82 (d, J=8.1 Hz, 1H), 6.71 (ddd, J=9.7, 8.9, 7.1 Hz, 1H), 4.62-4.42 (m, 2H), 4.31-4.08 (m, 2H), 3.94-3.82 (m, 2H), 3.25-3.07 (m, 2H), 2.80 (dq, J=7.0, 3.2 Hz, 3H), 2.23-1.94 (m, 3H), 1.35 (d, J=6.0 Hz, 3H), 1.19 (d, J=6.0 Hz, 3H).

Synthesis of 4-{2-[(2R)-4-[(4R)-7,8-difluoro-3,4-dihydro-2H-1-benzopyran-4-yl]-2-(2-isopropoxyphenyl)piperazin-1-yl]-7-azaspiro[3.5]nonan-7-yl}-2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-N-[(3R)-5-nitro-3-(oxan-4-yl)-3,4-dihydro-2H-1,4-benzoxazin-7-ylsulfonyl]benzamide (assumed): Into an 8 mL vial were added (3R)-1-[(4R)-7,8-difluoro-3,4-dihydro-2H-1-benzopyran-4-yl]-3-(2-isopropoxyphenyl)piperazine (assumed) (20 mg, 0.05 mmol, 1.0 eq), 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-N-[(3R)-5-nitro-3-(oxan-4-yl)-3,4-dihydro-2H-1,4-benzoxazin-7-ylsulfonyl]-4-{2-oxo-7-azaspiro[3.5]nonan-7-yl}benzamide (42 mg, 0.05 mmol, 1.0 eq), NaBH₃CN (16 mg, 0.3 mmol, 5.0 eq), ZnCl₂ (35 mg, 0.3 mmol, 5.0 eq) and MeOH (3 mL). The resulting mixture was stirred for overnight at 70° C. The reaction was quenched by the addition of water (5 mL) and extracted with CH₂Cl₂ (3×5 mL). The combined organic layers were washed with brine (5 mL), dried over anhydrous Na₂SO₄. After filtration, the filtrate was concentrated under vacuum. The residue was purified by reversed-phase flash chromatography using the following conditions: column, C18 silica gel; mobile phase, MeCN in water (0.1% FA), 10% to 50% gradient in 10 min; detector, UV 254 nm. Finally, 4-{2-[(2R)-4-[(4R)-7,8-difluoro-3,4-dihydro-2H-1-benzopyran-4-yl]-2-(2-isopropoxyphenyl)piperazin-1-yl]-7-azaspiro[3.5]nonan-7-yl}-2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-N-[(3R)-5-nitro-3-(oxan-4-yl)-3,4-dihydro-2H-1,4-benzoxazin-7-ylsulfonyl]benzamide (assumed) was obtained as a yellow solid (11 mg, 18.0%). LC-MS (ES, m/z) M+1:1188. ¹H NMR (300 MHz, Chloroform-d) δ 12.64 (s, 1H), 8.44 (d, J=9.1 Hz, 2H), 8.31 (d, J=2.1 Hz, 1H), 8.13 (d, J=9.1 Hz, 1H), 7.53 (s, 1H), 7.46 (d, J=2.1 Hz, 1H), 7.21 (s, 2H), 7.14-7.07 (m, 1H), 6.85 (d, J=9.1 Hz, 2H), 6.68 (d, J=10.3 Hz, 2H), 6.44 (d, J=2.4 Hz, 1H), 6.14 (dd, J=3.5, 2.0 Hz, 1H), 4.50-4.44 (m, 4H), 4.27-3.93 (m, 6H), 3.86-3.81 (m, 1H), 3.45-3.26 (m, 6H), 3.08-2.96 (m, 5H), 2.66-2.58 (m, 2H), 2.09-2.01 (m, 4H), 1.96-1.60 (m, 13H), 1.53-1.47 (m, 4H), 1.37-1.34 (m, 7H).

### Example 213 Preparation of 4-{2-[(2R)-4-[(4S)-7,8-difluoro-3,4-dihydro-2H-1-benzopyran-4-yl]-2-(2-isopropoxyphenyl)piperazin-1-yl]-7-azaspiro[3.5]nonan-7-yl}-2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-N-[(3R)-5-nitro-3-(oxan-4-yl)-3,4-dihydro-2H-1,4-benzoxazin-7-ylsulfonyl]benzamide (assumed)

Synthesis of 4-{2-[(2R)-4-[(4S)-7,8-difluoro-3,4-dihydro-2H-1-benzopyran-4-yl]-2-(2-isopropoxyphenyl)piperazin-1-yl]-7-azaspiro[3.5]nonan-7-yl}-2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-N-[(3R)-5-nitro-3-(oxan-4-yl)-3,4-dihydro-2H-1,4-benzoxazin-7-ylsulfonyl]benzamide (assumed): Into an 8 mL vial were added (3R)-1-[(4S)-7,8-difluoro-3,4-dihydro-2H-1-benzopyran-4-yl]-3-(2-isopropoxyphenyl)piperazine (assumed) (20 mg, 0.05 mmol, 1.0 eq), 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-N-[(3R)-5-nitro-3-(oxan-4-yl)-3,4-dihydro-2H-1,4-benzoxazin-7-ylsulfonyl]-4-{2-oxo-7-azaspiro[3.5]nonan-7-yl}benzamide (42 mg, 0.05 mmol, 1.0 eq), NaBH₃CN (16 mg, 0.3 mmol, 5.0 eq), ZnCl₂ (35 mg, 0.3 mmol, 5.0 eq) and MeOH (3 mL). The resulting mixture was stirred for overnight at 70° C. The reaction was quenched by the addition of water (5 mL) and extracted with CH₂Cl₂ (3×10 mL). The combined organic layers were washed with brine (10 mL), dried over anhydrous Na₂SO₄. After filtration, the filtrate was concentrated under vacuum. The residue was purified by reversed-phase flash chromatography using the following conditions: column, C18 silica gel; mobile phase, MeCN in water (0.1% FA), 10% to 50% gradient in 10 min; detector, UV 254 nm. Finally, 4-{2-[(2R)-4-[(4S)-7,8-difluoro-3,4-dihydro-2H-1-benzopyran-4-yl]-2-(2-isopropoxyphenyl)piperazin-1-yl]-7-azaspiro[3.5]nonan-7-yl}-2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-N-[(3R)-5-nitro-3-(oxan-4-yl)-3,4-dihydro-2H-1,4-benzoxazin-7-ylsulfonyl]benzamide (assumed) was obtained as a yellow solid (12 mg, 19.7%). LC-MS (ES, m/z) M+1: 1187. ¹H NMR (300 MHz, Chloroform-d) δ 12.64 (s, 1H), 8.45 (s, 2H), 8.31 (d, J=2.1 Hz, 1H), 8.13 (d, J=9.1 Hz, 1H), 7.46 (d, J=2.2 Hz, 2H), 7.21 (s, 2H), 7.14-7.06 (m, 1H), 6.84 (t, J=10.9 Hz, 1H), 6.70 (s, 1H), 6.67-6.57 (m, 1H), 6.44 (d, J=2.4 Hz, 1H), 6.14 (dd, J=3.5, 2.0 Hz, 1H), 4.53-4.49 (m, 1H), 4.48-4.45 (m, 4H), 4.27-4.02 (m, 6H), 3.98-3.88 (m, 1H), 3.86-3.68 (m, 2H), 3.45-3.35 (m, 8H), 3.10-3.05 (m, 8H), 2.94-2.87 (m, 2H), 2.44-2.02 (m, 7H) 1.67 (d, J=15.4 Hz, 3H), 1.36 (d, J=5.9 Hz, 4H), 1.31-1.19 (m, 5H).

### Example 214 Preparation of 4-(2-((R)-4-((S)-6,8-difluorochroman-4-yl)-2-(2-isopropoxyphenyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide (assumed)

Synthesis of (R)-1-((S)-6,8-difluorochroman-4-yl)-3-(2-isopropoxyphenyl)piperazine (assumed) and (R)-1-((R)-6,8-difluorochroman-4-yl)-3-(2-isopropoxyphenyl)piperazine (assumed): Into a 40 mL vial were added 4-chloro-6,8-difluorochromane (400 mg, 2.9 mmol, 1.0 eq), (2R)-2-(2-isopropoxyphenyl)piperazine (646 mg, 2.9 mmol, 1.0 eq), K₂CO₃ (1215 mg, 8.7 mmol, 3.0 eq) and MeCN (6 mL). The resulting mixture was stirred for additional 4 hours at 80° C. The reaction was quenched with water, and then extracted with CH₂Cl₂ (5 mL). The combined organic layers were washed with brine (3×5 mL), dried over anhydrous Na₂SO₄. After filtration, the filtrate was concentrated under vacuum. The crude residue was purified by Prep-TLC (EA) to give (R)-1-((S)-6,8-difluorochroman-4-yl)-3-(2-isopropoxyphenyl)piperazine (assumed) as a light yellow solid (88 mg, 8.1%) and (R)-1-((R)-6,8-difluorochroman-4-yl)-3-(2-isopropoxyphenyl)piperazine (assumed) as a light yellow solid (140 mg, 12.9%). LC-MS (ES, m/z) M+1: 389.

Synthesis of 4-(2-((R)-4-((S)-6,8-difluorochroman-4-yl)-2-(2-isopropoxyphenyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide (assumed): Into an 8 mL vial were added (R)-1-((S)-6,8-difluorochroman-4-yl)-3-(2-isopropoxyphenyl)piperazine (assumed) (9 mg, 0.1 mmol, 1.0 eq), 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-N-[(3R)-5-nitro-3-(oxan-4-yl)-3,4-dihydro-2H-1,4-benzoxazin-7-ylsulfonyl]-4-{2-oxo-7-azaspiro[3.5]nonan-7-yl}benzamide (18 mg, 0.1 mmol, 1.0 eq), NaBH₃CN (7 mg, 0.1 mmol, 5.0 eq), ZnCl₂ (15 mg, 0.1 mmol, 5.0 eq), and MeOH (1 mL). The resulting mixture was stirred for additional 4 hours at 70° C. The reaction was quenched with water, and then extracted with CH₂Cl₂ (5 mL). The combined organic layers were washed with brine (3×5 mL), dried over anhydrous Na₂SO₄. After filtration, the filtrate was concentrated under vacuum. The crude residue was purified by Prep-TLC (CH₂Cl₂/MeOH 18:1) to give 4-(2-((R)-4-((S)-6,8-difluorochroman-4-yl)-2-(2-isopropoxyphenyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide (assumed) as a yellow solid (10 mg, 36.8%). LC-MS (ESI, m/z) M+1: 1188. ¹H NMR (300 MHz, Chloroform-d) δ 12.64 (s, 1H), 8.46 (s, 1H), 8.39 (s, 1H), 8.31 (d, J=2.2 Hz, 1H), 8.13-8.10 (m, 1H), 7.46 (d, J=2.2 Hz, 2H), 7.17 (s, 2H), 7.10 (t, J=2.9 Hz, 1H), 6.86-6.83 (m, 3H), 6.72-6.68 (m, 2H), 6.44 (s, 1H), 6.14-6.12 (m, 1H), 4.61-4.46 (m, 2H), 4.45-4.41 (m, 2H), 4.07-3.94 (m, 8H), 3.45-3.38 (m, 7H), 3.09-3.00 (m, 5H), 2.95 (s, 1H), 2.63-2.59 (m, 2H), 2.45 (s, 1H), 2.17 (s, 2H), 2.03 (s, 1H), 1.79-1.69 (m, 6H), 1.66-1.60 (m, 6H), 1.49-1.43 (m, 1H), 1.42-1.25 (m, 7H), 1.22-1.17 (m, 1H).

### Example 215 Preparation of 4-(2-((R)-4-((R)-6,8-difluorochroman-4-yl)-2-(2-isopropoxyphenyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide (assumed)

Synthesis of 4-(2-((R)-4-((R)-6,8-difluorochroman-4-yl)-2-(2-isopropoxyphenyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide (assumed): Into an 8 mL vial were added (R)-1-((R)-6,8-difluorochroman-4-yl)-3-(2-isopropoxyphenyl)piperazine (assumed) (9 mg, 0.1 mmol, 1.0 eq), 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-N-[(3R)-5-nitro-3-(oxan-4-yl)-3,4-dihydro-2H-1,4-benzoxazin-7-ylsulfonyl]-4-{2-oxo-7-azaspiro[3.5]nonan-7-yl}benzamide (18 mg, 0.1 mmol, 1.0 eq), NaBH₃CN (7 mg, 0.1 mmol, 5.0 eq), ZnCl₂ (15 mg, 0.1 mmol, 5.0 eq) and MeOH (1 mL). The resulting mixture was stirred for additional 4 hours at 70° C. The reaction was quenched with water, and then extracted with CH₂Cl₂ (5 mL). The combined organic layers were washed with brine (3×5 mL), dried over anhydrous Na₂SO₄. After filtration, the filtrate was concentrated under vacuum. The crude residue was purified by Prep-TLC (CH₂Cl₂/MeOH=19:1) to give 4-(2-((R)-4-((R)-6,8-difluorochroman-4-yl)-2-(2-isopropoxyphenyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide as a yellow solid (10 mg, 36.3%). LC-MS (ESI, m/z) M+1: 1187. ¹H NMR (300 MHz, Chloroform-d) δ 12.64 (s, 1H), 8.45 (s, 2H), 8.31 (d, J=2.1 Hz, 1H), 8.13-8.10 (m, 1H), 7.46 (d, J=2.2 Hz, 1H), 7.38 (s, 1H), 7.12-7.10 (m, 3H), 6.83-6.79 (m, 2H), 6.70 (s, 2H), 6.45 (s, 1H), 6.14 (s, 1H), 4.42 (s, 2H), 4.25-3.93 (m, 7H), 3.78 (s, 1H), 3.51-3.46 (m, 1H), 3.45 (s, 4H), 3.39-3.34 (m, 2H), 3.09-3.01 (m, 6H), 2.89 (s, 2H), 2.37 (s, 1H), 1.77-1.69 (m, 8H), 1.63-1.53 (m, 9H), 1.38-1.25 (m, 2H), 1.20-1.14 (m, 3H), 0.91-0.86 (m, 4H) 0.79-0.75 (m, 1H).

### Example 216 Preparation of 4-(2-((R)-4-((R)-6,7-difluoroisochroman-4-yl)-2-(2-isopropoxyphenyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide (assumed)

Synthesis of 4,5-difluoro-2-iodobenzoic acid: Into a 1000 mL 3-necked round-bottom flask were added 2-amino-4,5-difluorobenzoic acid (20.0 g, 115.5 mmol, 1.0 eq), H₂O (140 mL) and H₂SO₄ (150 mL) at 25° C. To the above mixture was added NaNO₂ (9.6 g, 138.6 mmol, 1.2 eq) (dissolved in H₂O (40 mL)) dropwise over 15 min at 0° C. The resulting mixture was stirred for 1.5 hours at 0° C. To the above mixture was added KI (95.9 g, 577.6 mmol, 5.0 eq) (dissolved in 80 mL H₂O) dropwise over 15 min at 0° C. The resulting mixture was stirred for additional 90 min at 90° C. The mixture was allowed to cool down to 25° C. The reaction was quenched by the addition of sat. Na₂S₂O₃ (aq.) (200 mL) at 25° C. The precipitated solids were collected by filtration and washed with water (2×100 mL). The residue was dissolved in NaOH (4 M, 100 mL) and filtered, the filter cake was washed with NaOH (4 M, 2×50 mL). The filtrate was acidified to pH=3 with HCl (4 M). The precipitated solids were collected by filtration. Finally, 4,5-difluoro-2-iodobenzoic acid as a white solid (27 g, 82.3%). ¹H NMR (300 MHz, DMSO-d₆) δ 13.62 (s, 1H), 8.10 (dd, J=10.1, 7.8 Hz, 1H), 7.83 (dd, J=11.2, 8.3 Hz, 1H).

Synthesis of (4,5-difluoro-2-iodophenyl)methanol: Into a 500 mL 3-necked round-bottom flask were added 4,5-difluoro-2-iodobenzoic acid (20.0 g, 70.4 mmol, 1.0 eq) and THE (120 mL). To the above mixture was added BH₃-Me₂S (88 mL, 176.1 mmol, 2 M in THF, 2.5 eq) dropwise over 20 min at 0° C. The resulting mixture was stirred for 2 hours at 70° C. The reaction was quenched by the addition of HCl (1 M, 50 mL) and then extracted with EtOAc (2×200 mL). The combined organic layers were washed with brine (200 mL), dried over anhydrous Na₂SO₄. After filtration, the filtrate was concentrated under vacuum. Finally, (4,5-difluoro-2-iodophenyl)methanol was obtained as a colorless oil (17.0 g, 89.4%). ¹H NMR (300 MHz, DMSO-d₆) δ 7.92 (dd, J=10.0, 7.9 Hz, 1H), 7.42 (dd, J=12.1, 8.5 Hz, 1H), 5.64 (bs, 1H), 4.34 (s, 2H).

Synthesis of 1,2-difluoro-4-iodo-5-[(prop-2-en-1-yloxy)methyl]benzene: Into a 500 mL 3-necked round-bottom flask were added (4,5-difluoro-2-iodophenyl)methanol (10.0 g, 37.0 mmol, 1.0 eq) and THE (200 mL). To the above mixture was added NaH (1.8 g, 74.1 mmol, 60%, 2.0 eq) in portions at 0° C. The resulting mixture was stirred for 30 min at 0° C. To the above mixture was added allyl bromide (5.4 g, 44.4 mmol, 1.2 eq) dropwise at 0° C. The resulting mixture was stirred for additional 1 hour at 25° C. The reaction was quenched by the addition of water (50 mL) and then extracted with EtOAc (2×400 mL). The combined organic layers were washed with brine (500 mL), dried over anhydrous Na₂SO₄. After filtration, the filtrate was concentrated under vacuum. Finally, 1,2-difluoro-4-iodo-5-[(prop-2-en-1-yloxy)methyl]benzene was obtained as a colorless oil (10.0 g, 87.1%). ¹H NMR (300 MHz, DMSO-d₆) δ 7.98 (t, J=9.0 Hz, 1H), 7.45 (dd, J=10.5, 9.6 Hz, 1H), 5.96 (ddt, J=17.3, 10.6, 5.4 Hz, 1H), 5.33 (dq, J=17.3, 1.8 Hz, 1H), 5.21 (dq, J=10.5, 1.5 Hz, 1H), 4.38 (s, 2H), 4.09 (dt, J=5.4, 1.5 Hz, 2H).

Synthesis of 6,7-difluoro-4-methylidene-1,3-dihydro-2-benzopyran: Into a 250 mL round-bottom flask were placed 1,2-difluoro-4-iodo-5-[(prop-2-en-1-yloxy)methyl]benzene (10.0 g, 32.3 mmol, 1.0 eq), DMF (100 mL), NaOAc (5.3 g, 64.5 mmol, 2.0 eq), PPh₃ (1.7 g, 6.5 mmol, 0.2 eq) and Pd(OAc)₂ (0.7 g, 3.2 mmol, 0.1 eq). The resulting mixture was stirred for 3 hours at 100° C. under nitrogen atmosphere. The reaction was quenched by the addition of water (20 mL) and then extracted with EtOAc (2×300 mL). The combined organic layers were washed with brine (500 mL), dried over anhydrous Na₂SO₄. After filtration, the filtrate was concentrated under vacuum. The residue was applied onto a silica gel column and eluted with ethyl acetate/petroleum ether=1:2 to give 6,7-difluoro-4-methylidene-1,3-dihydro-2-benzopyran as a colorless oil (1.7 g, 28.9%). ¹H NMR (300 MHz, Chloroform-d) δ 7.47 (dd, J=11.5, 7.8 Hz, 1H), 6.91-6.80 (m, 1H), 5.52 (s, 1H), 5.06 (d, J=1.4 Hz, 1H), 4.75 (d, J=1.5 Hz, 2H), 4.43 (t, J=1.3 Hz, 2H).

Synthesis of 6,7-difluoro-1,3-dihydro-2-benzopyran-4-one: Into a 100 mL round-bottom flask were added 6,7-difluoro-4-methylidene-1,3-dihydro-2-benzopyran (1.9 g, 10.4 mmol, 1.0 eq), t-BuOH (12 mL), THE (12 mL) and H₂O (2 mL). To the above mixture was added K₂OsO₄.2H₂O (2.7 g, 7.3 mmol, 0.7 eq) and NalO₄ (6.7 g, 31.3 mmol, 3.0 eq) in portions at 25° C. The resulting mixture was stirred for 16 hours at 25° C. The reaction was quenched by the addition of sat. NaHSO₃ (aq.) (20 mL) at 0° C. The resulting mixture was extracted with EtOAc (2×100 mL). The combined organic layers were washed with brine (100 mL), dried over anhydrous Na₂SO₄. After filtration, the filtrate was concentrated under vacuum. The residue was applied onto a silica gel column and eluted with ethyl acetate/petroleum ether=1:1 to give 6,7-difluoro-1,3-dihydro-2-benzopyran-4-one as a colorless oil (900 mg, 46.9%). ¹H NMR (300 MHz, Chloroform-d) δ 7.88 (dd, J=10.0, 7.9 Hz, 1H), 7.07 (dd, J=9.8, 6.9 Hz, 1H), 4.86 (s, 2H), 4.38 (s, 2H).

Synthesis of 6,7-difluoro-3,4-dihydro-1H-2-benzopyran-4-ol: Into a 100 mL round-bottom flask were added 6,7-difluoro-1,3-dihydro-2-benzopyran-4-one (900 mg, 4.9 mmol, 1.0 eq) and MeOH (20 mL). To the above mixture was added NaBH₄ (555 mg, 14.7 mmol, 3.0 eq) in portions at 0° C. The resulting mixture was stirred for 2 hours at 25° C. The reaction was quenched by the addition of water (20 mL), and then neutralized to pH=7 with HCl (1 M), and then extracted with CH₂Cl₂ (2×100 mL). The combined organic layers were washed with brine (100 mL), dried over anhydrous Na₂SO₄. After filtration, the filtrate was concentrated under vacuum. Finally, 6,7-difluoro-3,4-dihydro-1H-2-benzopyran-4-ol was obtained as a colorless oil (800 mg, crude).

Synthesis of 4-chloro-6,7-difluoro-3,4-dihydro-1H-2-benzopyran: Into a 50 mL round-bottom flask were added 6,7-difluoro-3,4-dihydro-1H-2-benzopyran-4-ol (800 mg, 4.3 mmol, 1.0 eq) and CHCl₃ (3 mL) at 25° C. To the above mixture was added SOCl₂ (15 mL, 206.8 mmol, 48.1 eq) dropwise at 0° C. The resulting mixture was stirred for 2 hours at 25° C. The reaction was quenched by the addition of water (20 mL), and then extracted with CH₂Cl₂ (2×50 mL). The combined organic layers were washed with saturated NaHCO₃ (aq.) (100 mL) and brine (100 mL), dried over anhydrous Na₂SO₄. After filtration, the filtrate was concentrated under vacuum. Finally, 4-chloro-6,7-difluoro-3,4-dihydro-1H-2-benzopyran as a colorless oil (650 mg, 73.9%). ¹H NMR (400 MHz, DMSO-d₆) δ 7.51 (dd, J=11.2, 8.0 Hz, 1H), 7.26 (dd, J=11.2, 8.0 Hz, 1H), 5.40-5.30 (m, 1H), 4.82 (d, J=15.5 Hz, 1H), 4.65 (d, J=15.5 Hz, 1H), 4.08 (d, J=2.9 Hz, 2H)

Synthesis of (3R)-1-[(4R)-6,7-difluoro-3,4-dihydro-1H-2-benzopyran-4-yl]-3-(2-isopropoxyphenyl)piperazine (assumed) and (3R)-1-[(4S)-6,7-difluoro-3,4-dihydro-1H-2-benzopyran-4-yl]-3-(2-isopropoxyphenyl)piperazine (assumed): Into a 50 mL round-bottom flask were added 4-chloro-6,7-difluoro-3,4-dihydro-1H-2-benzopyran (500 mg, 2.4 mmol, 1.0 eq), MeCN (10 mL), K₂CO₃ (1013 mg, 7.3 mmol, 3.0 eq) and (2R)-2-(2-isopropoxyphenyl)piperazine (assumed) (538.4 mg, 2.4 mmol, 1.0 eq) at 25° C. The resulting mixture was stirred for 16 hours at 80° C. The reaction was quenched by the addition of water (5 mL), and then extracted with EtOAc (2×20 mL). The combined organic layers were washed with brine (20 mL), dried over anhydrous Na₂SO₄. After filtration, the filtrate was concentrated under vacuum. The residue was purified by Prep-TLC (100% EA) to afford (3R)-1-[(4R)-6,7-difluoro-3,4-dihydro-1H-2-benzopyran-4-yl]-3-(2-isopropoxyphenyl)piperazine (assumed) as a colorless oil (150 mg, 15.8%) and (3R)-1-[(4S)-6,7-difluoro-3,4-dihydro-1H-2-benzopyran-4-yl]-3-(2-isopropoxyphenyl)piperazine (assumed) as a colorless oil (150 mg, 15.8%).

Synthesis of 4-(2-((R)-4-((R)-6,7-difluoroisochroman-4-yl)-2-(2-isopropoxyphenyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide (assumed): Into a 2 mL sealed-tube, were placed (3R)-1-[(4R)-6,7-difluoro-3,4-dihydro-1H-2-benzopyran-4-yl]-3-(2-isopropoxyphenyl)piperazine (10 mg, 0.03 mmol, 1.0 eq), 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-N-[(3R)-5-nitro-3-(oxan-4-yl)-3,4-dihydro-2H-1,4-benzoxazin-7-ylsulfonyl]-4-{2-oxo-7-azaspiro[3.5]nonan-7-yl}benzamide (20 mg, 0.03 mmol, 1.0 eq), NaBH₃CN (8 mg, 0.1 mmol, 5.0 eq), ZnCl₂ (17 mg, 0.1 mmol, 5.0 eq) and MeOH (0.5 mL). The resulting mixture was stirred for 5 hours at 70° C. The resulting solution was diluted with dichloromethane/methanol=10:1 (2×10 mL) and then washed with brine (2×10 mL). The combined organic layers was dried over anhydrous Na₂SO₄. After filtration, the filtrate was concentrated under vacuum. The crude product was purified by Prep-HPLC using the following conditions (Column: XBridge Prep OBD C18 Column, 30×150 mm, 5 μm; Mobile Phase A: water (0.05% NH₃·H₂O), Mobile Phase B: MeCN; Flow rate: 60 mL/min; Gradient: 25% B to 55% B in 8 min, 55% B; Wave Length: 254/220 nm; RT1(min): 6.7) to afford 4-(2-((R)-4-((R)-6,7-difluoroisochroman-4-yl)-2-(2-isopropoxyphenyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide (assumed) as a light yellow solid (8 mg, 27.4%). LC-MS (ES, m/z) M+1: 1188. ¹H NMR (400 MHz, DMSO-d₆) δ 12.75 (s, 1H), 11.14 (s, 1H), 8.85 (s, 1H), 7.96-7.94 (m, 1H), 7.58-7.55 (m, 1H), 7.41-7.39 (m, 2H), 7.16-7.14 (m, 2H), 7.07-7.05 (m, 1H), 6.97-6.94 (m, 2H), 6.85-6.83 (m, 1H), 6.79-6.75 (m, 1H), 6.43-6.40 (m, 1H), 5.99-5.96 (m, 1H), 4.64-4.62 (m, 2H), 4.48-4.45 (m, 1H), 4.29-4.25 (m, 1H), 4.23-4.19 (m, 1H), 4.12-4.10 (m, 2H), 4.05-3.97 (m, 3H), 3.88-3.85 (m, 2H), 3.79-3.75 (m, 5H), 3.70-3.60 (m, 2H), 3.56-3.45 (m, 3H), 3.25-2.80 (m, 8H), 2.62-2.58 (m, 1H), 1.95-1.90 (m, 1H), 1.83-1.57 (m, 5H), 1.30-1.20 (m, 14H), 1.10-1.00 (m, 1H).

### Example 217 Preparation of 4-(2-((R)-4-((S)-6,7-difluoroisochroman-4-yl)-2-(2-isopropoxyphenyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide (assumed)

Synthesis of 4-(2-((R)-4-((S)-6,7-difluoroisochroman-4-yl)-2-(2-isopropoxyphenyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide (assumed): Into a 2 mL sealed-tube, were placed (3R)-1-[(4S)-6,7-difluoro-3,4-dihydro-1H-2-benzopyran-4-yl]-3-(2-isopropoxyphenyl)piperazine (10 mg, 0.03 mmol, 1.0 eq), 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-N-[(3R)-5-nitro-3-(oxan-4-yl)-3,4-dihydro-2H-1,4-benzoxazin-7-ylsulfonyl]-4-{2-oxo-7-azaspiro[3.5]nonan-7-yl}benzamide (20 mg, 0.03 mmol, 1.0 eq), NaBH₃CN (8 mg, 0.1 mmol, 5.0 eq), ZnCl₂ (17 mg, 0.1 mmol, 5.0 eq) and MeOH (0.5 mL). The resulting mixture was stirred for 5 hours at 70° C. The resulting solution was diluted with dichloromethane/methanol=10:1 (2×10 mL) and washed with brine (2×10 mL). The combined organic layers was dried over anhydrous Na₂SO₄. After filtration, the filtrate was concentrated under vacuum. The crude product was purified by Prep-HPLC using the following conditions (Column: XBridge Prep OBD C18 Column, 30×150 mm, 5 μm; Mobile Phase A: water (0.05% NH₃·H₂O), Mobile Phase B: MeCN; Flow rate: 60 mL/min; Gradient: 25% B to 55% B in 8 min, 55% B; Wave Length: 254/220 nm; RT1(min): 6.7) to afford 4-(2-((R)-4-((S)-6,7-difluoroisochroman-4-yl)-2-(2-isopropoxyphenyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide (assumed) as a light yellow solid (8 mg, 27.4%). LC-MS (ES, m/z) M+1: 1188. ¹H NMR (400 MHz, DMSO-d₆) δ 12.75 (s, 1H), 11.14 (s, 1H), 8.85 (s, 1H), 7.96-7.94 (m, 1H), 7.58-7.55 (m, 1H), 7.41-7.39 (m, 2H), 7.16-7.14 (m, 2H), 7.07-7.05 (m, 1H), 6.97-6.94 (m, 2H), 6.85-6.83 (m, 1H), 6.79-6.75 (m, 1H), 6.43-6.40 (m, 1H), 5.99-5.96 (m, 1H), 4.64-4.62 (m, 2H), 4.48-4.45 (m, 1H), 4.29-4.25 (m, 1H), 4.23-4.19 (m, 1H), 4.12-4.10 (m, 2H), 4.05-3.97 (m, 3H), 3.88-3.85 (m, 2H), 3.79-3.75 (m, 5H), 3.70-3.60 (m, 2H), 3.56-3.45 (m, 3H), 3.25-2.80 (m, 8H), 2.62-2.58 (m, 1H), 1.95-1.90 (m, 1H), 1.83-1.57 (m, 5H), 1.30-1.20 (m, 14H), 1.10-1.00 (m, 1H).

### Example 218 Preparation of 4-(2-((R)-4-((S)-6-fluoro-7-((S)-3-methylmorpholino)-3,4-dihydro-2H-pyrano[2,3-b]pyridin-4-yl)-2-(2-isopropoxyphenyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide (assumed)

Synthesis of (2,6-dichloro-5-fluoropyridin-3-yl)methanol: Into a 1000 mL 3-necked round-bottom flask were placed 2,6-dichloro-5-fluoropyridine-3-carboxylic acid (25.0 g, 119.1 mmol, 1.0 eq) and THE (100 mL) at 25° C. To the above mixture was added BH₃-THF (357 mL, 1 N, 3.0 eq) dropwise at 0° C. The resulting mixture was stirred for 16 hours at 25° C. The reaction was quenched by the addition of HCl (1 M, 50 mL), and then extracted with EtOAc (2×500 mL). The combined organic layers were washed with brine (500 mL), dried over anhydrous Na₂SO₄. After filtration, the filtrate was concentrated under vacuum. This resulted in (2,6-dichloro-5-fluoropyridin-3-yl)methanol as a colorless oil (25.0 g, crude). LC-MS (ES, m/z) M+1: 196/198.

Synthesis of 2,6-dichloro-5-fluoropyridine-3-carbaldehyde: Into a 1000 mL 3-necked round-bottom flask were added (2,6-dichloro-5-fluoropyridin-3-yl)methanol (25.0 g, 127.6 mmol, 1.0 eq) and CH₂Cl₂ (500 mL) at 25° C. To the above mixture was added Dess-Martin Periodinane (81.2 g, 191.3 mmol, 1.5 eq) in portions over 10 min at 0° C. The resulting mixture was stirred for 2 hours at 25° C. The reaction was quenched by the addition of saturated NaHSO₃ (aq.) (100 mL), and then extracted with CH₂Cl₂ (2×800 mL). The combined organic layers were washed with saturated NaHCO₃ (aq.) (800 mL) and brine (800 mL), dried over anhydrous Na₂SO₄. After filtration, the filtrate was concentrated under vacuum. Finally, 2,6-dichloro-5-fluoropyridine-3-carbaldehyde was obtained as a colorless oil (18.0 g, crude).

Synthesis of ethyl 3-(2,6-dichloro-5-fluoropyridin-3-yl)acrylate: Into a 1000 mL 3-necked round-bottom flask were added triethyl phosphonoacetate (25.0 g, 111.3 mmol, 1.2 eq) and THE (300 mL) at 25° C. To the above mixture was added NaH (4.5 g, 111.3 mmol, 1.2 eq 60%) in portions at 0° C. The resulting mixture was stirred for 30 min at 0° C. To the above mixture was added 2,6-dichloro-5-fluoropyridine-3-carbaldehyde (18.0 g, 92.8 mmol, 1.0 eq) in portions at 0° C. The resulting mixture was stirred for additional 16 hours at 25° C. The reaction was quenched by the addition of water/ice (50 mL), and then extracted with EtOAc (2×500 mL). The combined organic layers were washed with brine (500 mL), dried over anhydrous Na₂SO₄. After filtration, the filtrate was concentrated under vacuum. Finally, ethyl 3-(2,6-dichloro-5-fluoropyridin-3-yl)acrylate as a colorless oil (30.0 g, crude).

Synthesis of ethyl 3-(2,6-dichloro-5-fluoropyridin-3-yl)propanoate: Into a 1000 mL round-bottom flask were added ethyl 3-(2,6-dichloro-5-fluoropyridin-3-yl)acrylate (30.0 g, 113.6 mmol, 1.0 eq), MeOH (300 mL) and Raney Ni (1.0 g, 11.4 mmol, 0.1 eq) at 25° C. The resulting mixture was stirred for 16 hours at 25° C. under hydrogen atmosphere. The resulting mixture was filtered, the filter cake was washed with MeOH (2×50 mL). The filtrate was concentrated under vacuum. The crude residue was applied onto a silica gel column and eluted with ethyl acetate/petroleum ether=1:1 to give ethyl 3-(2,6-dichloro-5-fluoropyridin-3-yl)propanoate as a colorless oil (15.0 g, 49.6%). ¹H NMR (400 MHz, DMSO-d₆) δ 8.08 (d, J=8.8 Hz, 1H), 4.07 (q, J=7.1 Hz, 2H), 2.95 (t, J=7.6 Hz, 2H), 2.70 (t, J=7.6 Hz, 2H), 1.17 (t, J=7.1 Hz, 3H).

Synthesis of 3-(2,6-dichloro-5-fluoropyridin-3-yl)propan-1-ol: Into a 500 mL 3-necked round-bottom flask were added ethyl 3-(2,6-dichloro-5-fluoropyridin-3-yl)propanoate (14.0 g, 52.6 mmol, 1.0 eq) and THE (150 mL) at 25° C. To the above mixture was added LiBH₄ (53 mL, 105.2 mmol, 2 M in THF, 2.0 eq) dropwise at 0° C. The resulting mixture was stirred for 6 hours at 25° C. The reaction was quenched by the addition of water (50 mL) at 0° C. The mixture was neutralized to pH=7 with HCl (1 M), and then extracted with EtOAc (2×500 mL). The combined organic layers were washed with brine (500 mL), dried over anhydrous Na₂SO₄. After filtration, the filtrate was concentrated under vacuum. Finally, 3-(2,6-dichloro-5-fluoropyridin-3-yl)propan-1-ol was obtained as a colorless oil (10.0 g, 84.8%). ¹H NMR (400 MHz, DMSO-d₆) δ 8.04 (d, J=8.7 Hz, 1H), 4.59 (t, J=5.1 Hz, 1H), 3.45 (td, J=6.2, 5.0 Hz, 2H), 2.77-2.68 (m, 2H), 1.80-1.68 (m, 2H).

Synthesis of 7-chloro-6-fluoro-2H,3H,4H-pyrano[2,3-b]pyridine: Into a 500 mL 3-necked round-bottom flask were placed 3-(2,6-dichloro-5-fluoropyridin-3-yl)propan-1-ol (9.0 g, 40.2 mmol, 1.0 eq) and THE (150 mL). To the above mixture was added NaH (4.8 g, 120.5 mmol, 60%, 3.0 eq) in portions at 0° C. The resulting mixture was stirred for additional 16 hours at 80° C. The reaction was quenched by the addition of water/ice (50 mL), and then extracted with EtOAc (2×200 mL). The combined organic layers were washed with HCl (0.05 M, 100 mL), saturated NaHCO₃ (aq.) (100 mL) and brine (100 mL), dried over anhydrous Na₂SO₄. After filtration, the filtrate was concentrated under vacuum. The crude residue was applied onto a silica gel column and eluted with ethyl acetate/petroleum ether=1:1 to give 7-chloro-6-fluoro-2H,3H,4H-pyrano[2,3-b]pyridine as a colorless oil (2.0 g, 26.5%). ¹H NMR (300 MHz, DMSO-d₆) δ 7.75 (dt, J=8.3, 1.1 Hz, 1H), 4.29 (dd, J=10.7, 4.9 Hz, 2H), 2.79 (t, J=6.4 Hz, 2H), 1.97-1.83 (m, 2H).

Synthesis of 4-bromo-7-chloro-6-fluoro-2H,3H,4H-pyrano[2,3-b]pyridine: Into a 50 mL round-bottom flask were added 7-chloro-6-fluoro-2H,3H,4H-pyrano[2,3-b]pyridine (1.2 g, 6.4 mmol, 1.0 eq), NBS (1.1 g, 6.4 mmol, 1.0 eq), BPO (330 mg, 1.3 mmol, 0.2 eq) and CCl₄ (20 mL) at 25° C. The resulting mixture was stirred for 2 hours at 80° C. The reaction was quenched by the addition of water (10 mL), and then extracted with EtOAc (2×50 mL). The combined organic layers were washed with brine (50 mL), dried over anhydrous Na₂SO₄. After filtration, the filtrate was concentrated under vacuum. The crude residue was applied onto a silica gel column and eluted with ethyl acetate/petroleum ether=1:1 to give 4-bromo-7-chloro-6-fluoro-2H,3H,4H-pyrano[2,3-b]pyridine as a white solid (1.0 g, 58.6%). ¹H NMR (400 MHz, DMSO-d₆) δ 8.03 (d, J=8.0 Hz, 1H), 5.77-5.70 (m, 1H), 4.63-4.48 (m, 2H), 2.55-2.43 (m, 1H), 2.32-2.30 (m, 1H).

Synthesis of (3R)-1-[(4S)-7-chloro-6-fluoro-2H,3H,4H-pyrano[2,3-b]pyridin-4-yl]-3-(2-isopropoxyphenyl)piperazine (assumed) and (3R)-1-[(4R)-7-chloro-6-fluoro-2H,3H,4H-pyrano[2,3-b]pyridin-4-yl]-3-(2-isopropoxyphenyl)piperazine (assumed): Into a 50 mL round-bottom flask were placed 4-bromo-7-chloro-6-fluoro-2H,3H,4H-pyrano[2,3-b]pyridine (600 mg, 2.3 mmol, 1.0 eq), DMF (10 mL), K₂CO₃ (934 mg, 6.8 mmol, 3.0 eq) and (2R)-2-(2-isopropoxyphenyl)piperazine (496 mg, 2.3 mmol, 1.0 eq) at 25° C. The resulting mixture was stirred for 3 hours at 25° C. The reaction was quenched by the addition of water (10 mL), and then extracted with EtOAc (2×20 mL). The combined organic layers were washed with brine (50 mL), dried over anhydrous Na₂SO₄. After filtration, the filtrate was concentrated under vacuum. The residue was purified by Prep-TLC (ethyl acetate/petroleum ether=1:0) to afford (3R)-1-[(4S)-7-chloro-6-fluoro-2H,3H,4H-pyrano[2,3-b]pyridin-4-yl]-3-(2-isopropoxyphenyl)piperazine (assumed) as a colorless oil (160 mg, 17.5%) and (3R)-1-[(4R)-7-chloro-6-fluoro-2H,3H,4H-pyrano[2,3-b]pyridin-4-yl]-3-(2-isopropoxyphenyl)piperazine (assumed) as a colorless oil (170 mg, 18.6%).

Synthesis of (3S)-4-[(4S)-6-fluoro-4-[(3R)-3-(2-isopropoxyphenyl)piperazin-1-yl]-2H,3H,4H-pyrano[2,3-b]pyridin-7-yl]-3-methylmorpholine (assumed): Into an 8 mL sealed tube were placed (3R)-1-[(4S)-7-chloro-6-fluoro-2H,3H,4H-pyrano[2,3-b]pyridin-4-yl]-3-(2-isopropoxyphenyl)piperazine (assumed) (100 mg, 0.2 mmol, 1.0 eq), t-BuOK (55 mg, 0.5 mmol, 2.0 eq), Pd-PEPPSI-IPentCl (21 mg, 0.03 mmol, 0.1 eq), dioxane (2 mL) and (3S)-3-methylmorpholine (150 mg, 1.5 mmol, 6.0 eq) at 25° C. The resulting mixture was stirred for 16 hours at 110° C. under nitrogen atmosphere. The reaction was quenched by the addition of water (2 mL) at 25° C. The resulting mixture was extracted with EtOAc (2×10 mL). The combined organic layers were washed with brine (20 mL), dried over anhydrous Na₂SO₄. After filtration, the filtrate was concentrated under vacuum. The residue was purified by Prep-TLC (CH₂Cl₂/MeOH=10:1) to afford (3S)-4-[(4S)-6-fluoro-4-[(3R)-3-(2-isopropoxyphenyl)piperazin-1-yl]-2H,3H,4H-pyrano[2,3-b]pyridin-7-yl]-3-methylmorpholine (assumed) as a colorless oil (60 mg, 51.8%).

Synthesis of 4-(2-((R)-4-((S)-6-fluoro-7-((S)-3-methylmorpholino)-3,4-dihydro-2H-pyrano[2,3-b]pyridin-4-yl)-2-(2-isopropoxyphenyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide (assumed): Into a 2 mL sealed-tube, were added (3S)-4-[(4S)-6-fluoro-4-[(3R)-3-(2-isopropoxyphenyl)piperazin-1-yl]-2H,3H,4H-pyrano[2,3-b]pyridin-7-yl]-3-methylmorpholine (assumed) (12 mg, 0.03 mmol, 1.0 eq), 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-N-[(3R)-5-nitro-3-(oxan-4-yl)-3,4-dihydro-2H-1,4-benzoxazin-7-ylsulfonyl]-4-{2-oxo-7-azaspiro[3.5]nonan-7-yl}benzamide (20 mg, 0.03 mmol, 1.0 eq), NaBH₃CN (8 mg, 0.1 mmol, 5.0 eq), ZnCl₂ (17 mg, 0.1 mmol, 5.0 eq) and MeOH (0.5 mL). The resulting mixture was stirred for 3 hours at 50° C. The resulting solution was extracted with dichloromethane/methanol=10:1 (2×10 mL) and washed with brine (2×10 mL) and the organic layers combined. The mixture was dried over anhydrous Na₂SO₄. After filtration, the filtrate was concentrated under vacuum. The crude product was purified by Prep-HPLC using the following conditions (Column: XBridge Prep OBD C18 Column, 30×150 mm, 5 μm; Mobile Phase A: water (0.05% NH3·H2O), Mobile Phase B: MeCN; Flow rate: 60 mL/min; Gradient: 25% B to 55% B in 8 min, 55% B; Wave Length: 254/220 nm;) to afford 4-(2-((R)-4-((S)-6-fluoro-7-((S)-3-methylmorpholino)-3,4-dihydro-2H-pyrano[2,3-b]pyridin-4-yl)-2-(2-isopropoxyphenyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide (assumed) as a light yellow solid (6 mg, 19.3%). LC-MS (ES, m/z) M+1: 1270. ¹H NMR (300 MHz, DMSO-d₆) δ 12.73 (s, 1H), 11.10 (s, 1H), 8.00-7.95 (m, 1H), 7.46-7.44 (m, 2H), 7.20-7.15 (m, 1H), 7.08-7.05 (m, 1H), 7.00-6.78 (m, 7H), 6.46-6.42 (m, 1H), 6.03-5.98 (m, 1H), 4.60-4.58 (m, 2H), 4.36-4.26 (m, 3H), 4.14-3.95 (m, 6H), 3.93-3.71 (m, 9H), 3.62-3.42 (m, 5H), 3.23-3.14 (m, 3H), 3.12-2.84 (m, 8H), 2.34-2.22 (m, 2H), 2.11-2.03 (m, 2H), 1.95-1.70 (m, 2H), 1.65-1.53 (m, 4H), 1.47-1.18 (m, 12H), 0.90-0.76 (m, 1H).

### Example 219 Preparation of 4-(2-((R)-4-((R)-6-fluoro-7-((S)-3-methylmorpholino)-3,4-dihydro-2H-pyrano[2,3-b]pyridin-4-yl)-2-(2-isopropoxyphenyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide (assumed)

Synthesis of (3S)-4-[(4R)-6-fluoro-4-[(3R)-3-(2-isopropoxyphenyl)piperazin-1-yl]-2H,3H,4H-pyrano[2,3-b]pyridin-7-yl]-3-methylmorpholine (assumed): Into an 8 mL sealed tube were placed (3R)-1-[(4R)-7-chloro-6-fluoro-2H,3H,4H-pyrano[2,3-b]pyridin-4-yl]-3-(2-isopropoxyphenyl)piperazine (assumed) (100 mg, 0.2 mmol, 1.0 eq), t-BuOK (55 mg, 0.5 mmol, 2.0 eq), Pd-PEPPSI-IPentCl (21 mg, 0.03 mmol, 0.1 eq), dioxane (2 mL) and (3S)-3-methylmorpholine (150 mg, 1.5 mmol, 6.0 eq) at 25° C. The resulting mixture was stirred for 16 hours at 110° C. under nitrogen atmosphere. The reaction was quenched by the addition of water (2 mL), and then extracted with EtOAc (2×10 mL). The combined organic layers were washed with brine (20 mL), dried over anhydrous Na₂SO₄. After filtration, the filtrate was concentrated under vacuum. The residue was purified by Prep-TLC (CH₂Cl₂/MeOH=10:1) to afford (3S)-4-[(4R)-6-fluoro-4-[(3R)-3-(2-isopropoxyphenyl)piperazin-1-yl]-2H,3H,4H-pyrano[2,3-b]pyridin-7-yl]-3-methylmorpholine (assumed) as a colorless oil (60 mg, 51.8%).

Synthesis of 4-(2-((R)-4-((R)-6-fluoro-7-((S)-3-methylmorpholino)-3,4-dihydro-2H-pyrano[2,3-b]pyridin-4-yl)-2-(2-isopropoxyphenyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide (assumed): Into a 2 mL sealed-tube, were added (3S)-4-[(4R)-6-fluoro-4-[(3R)-3-(2-isopropoxyphenyl)piperazin-1-yl]-2H,3H,4H-pyrano[2,3-b]pyridin-7-yl]-3-methylmorpholine (assumed) (12 mg, 0.03 mmol, 1.0 eq), 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-N-[(3R)-5-nitro-3-(oxan-4-yl)-3,4-dihydro-2H-1,4-benzoxazin-7-ylsulfonyl]-4-{2-oxo-7-azaspiro[3.5]nonan-7-yl}benzamide (20 mg, 0.03 mmol, 1.0 eq), NaBH₃CN (8 mg, 0.1 mmol, 5.0 eq), ZnCl₂ (17 mg, 0.1 mmol, 5.0 eq) and MeOH (0.5 mL). The resulting mixture was stirred for 3 hours at 50° C. The resulting solution was diluted with dichloromethane/methanol=10:1 (2×10 mL), and then washed with brine (2×10 mL). The combined organic layers was dried over anhydrous Na₂SO₄. After filtration, the filtrate was concentrated under vacuum. The crude product was purified by Prep-H PLC using the following conditions (Column: XBridge Prep OBD C18 Column, 30×150 mm, 5 μm; Mobile Phase A: water (0.05% NH3·H2O), Mobile Phase B: MeCN; Flow rate: 60 mL/min; Gradient: 25% B to 55% B in 8 min, 55% B; Wave Length: 254/220 nm;) to afford 4-(2-((R)-4-((R)-6-fluoro-7-((S)-3-methylmorpholino)-3,4-dihydro-2H-pyrano[2,3-b]pyridin-4-yl)-2-(2-isopropoxyphenyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide (assumed) as a light yellow solid (6 mg, 19.3%). LC-MS (ES, m/z) M+1: 1270. ¹H NMR (300 MHz, DMSO-d₆) δ 12.75 (s, 1H), 11.13 (s, 1H), 8.83 (s, 1H), 7.97-7.95 (m, 1H), 7.77-7.75 (m, 1H), 7.46-7.43 (m, 1H), 7.37-7.33 (m, 1H), 7.07-7.05 (m, 2H), 6.99-6.96 (m, 1H), 6.91-6.89 (m, 2H), 6.82-6.80 (m, 2H), 6.46-6.44 (m, 1H), 6.02-6.00 (m, 1H), 4.60-4.58 (m, 2H), 4.36-4.26 (m, 3H), 4.14-3.95 (m, 6H), 3.93-3.71 (m, 9H), 3.62-3.42 (m, 5H), 3.23-3.14 (m, 3H), 3.12-2.84 (m, 8H), 2.34-2.22 (m, 2H), 2.11-2.03 (m, 2H), 1.95-1.70 (m, 2H), 1.65-1.53 (m, 4H), 1.47-1.18 (m, 12H), 0.90-0.76 (m, 1H).

### Example 220 Preparation of 4-(2-((R)-4-((S)-6,8-difluorochroman-3-yl)-2-(2-isopropoxyphenyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide (assumed)

Synthesis of 3,5-difluoro-2-hydroxybenzaldehyde: Into a 500 mL round-bottom flask were placed 2,4-difluorophenol (20.0 g, 153.7 mmol, 1.0 eq), TFA (150 mL) and 1,3,5,7-tetraazatricyclo[3.3.1.1\hat{ }{3,7}]decane (43.1 g, 307.5 mmol, 2.0 eq) at 25° C. The resulting mixture was stirred for 16 hours at 110° C. The mixture was allowed to cool down to 25° C. To the above mixture was added H₂SO₄ (225 mL, 50%) and H₂O (110 mL) at 25° C. The resulting mixture was stirred for additional 2 hours at 25° C. The resulting mixture was extracted with EtOAc (2×500 mL). The combined organic layers were washed with HCl (1 N) (2×300 mL) and brine (500 mL), dried over anhydrous Na₂SO₄. After filtration, the filtrate was concentrated under vacuum. Finally, 3,5-difluoro-2-hydroxybenzaldehyde (12 g, 49.4%) was obtained as a white solid. ¹H NMR (400 MHz, DMSO-d₆) δ 10.87 (s, 1H), 10.27 (d, J=2.8 Hz, 1H), 7.63-7.60 (m, 1H), 7.25 (ddt, J=8.5, 3.1, 1.6 Hz, 1H).

Synthesis of 6,8-difluoro-3-nitro-2H-chromene: Into a 1000 mL 3-necked round-bottom flask were placed 3,5-difluoro-2-hydroxybenzaldehyde (10.0 g, 63.3 mmol, 1.0 eq), n-BuNH₂ (2.3 g, 31.6 mmol, 0.5 eq), phthalic anhydride (18.7 g, 126.5 mmol, 2.0 eq), 2-nitroethanol (5.8 g, 63.3 mmol, 1.0 eq) and toluene (500 mL) at 25° C. The resulting mixture was stirred for 24 hours at 110° C. The mixture was allowed to cool down to 25° C. To the above mixture was added 2-nitroethanol (5.8 g, 63.3 mmol, 1.0 eq) at 25° C. The resulting mixture was stirred for additional 24 hours at 110° C. The resulting mixture was concentrated under vacuum. The residue was applied onto a silica gel column and eluted with ethyl acetate/petroleum ether=1:1 to give 5.0 g crude product. The residue was dissolved in ethyl acetate (100 mL). The resulting mixture was washed with NaOH (2%, 2×100 mL) and water (2×100 mL). The combined organic layers were dried over anhydrous Na₂SO₄. After filtration, the filtrate was concentrated under vacuum. Finally, 6,8-difluoro-3-nitro-2H-chromene was obtained as a yellow solid (3.5 g, 26.0%). ¹H NMR (400 MHz, DMSO-d₆) δ 8.06 (d, J=1.7 Hz, 1H), 7.47 (ddd, J=11.4, 8.9, 3.0 Hz, 1H), 7.33 (ddd, J=8.3, 3.0, 1.7 Hz, 1H), 5.32 (d, J=1.3 Hz, 2H).

Synthesis of 6,8-difluoro-2,4-dihydro-1-benzopyran-3-one: Into a 250 mL round-bottom flask were added Fe (8.7 g, 154.8 mmol, 10.0 eq) and AcOH (50 mL). To the above mixture was added 6,8-difluoro-3-nitro-2H-chromene (3.3 g, 15.5 mmol, 1.0 eq) in portions over 10 min at 60° C. The resulting mixture was stirred for 2 hours at 60° C. The mixture was allowed to cool down to 25° C. and stirred for additional 16 hours at this temperature. The reaction was quenched by the addition of water/ice (50 mL) at 0° C. The resulting mixture was filtered, the filter cake was washed with CH₂Cl₂ (2×100 mL). The filtrate was washed with water (200 mL) and brine (200 mL), dried over anhydrous Na₂SO₄. After filtration, the filtrate was concentrated under vacuum. Finally, 6,8-difluoro-2,4-dihydro-1-benzopyran-3-one was obtained as a colorless oil (1.8 g, crude).

Synthesis of (3R)-1-(6,8-difluoro-3,4-dihydro-2H-1-benzopyran-3-yl)-3-(2-isopropoxyphenyl)piperazine (assumed): Into a 100 mL round-bottom flask were added 6,8-difluoro-2,4-dihydro-1-benzopyran-3-one (1.7 g, 4.6 mmol, 1.0 eq, 50%), (2R)-2-(2-isopropoxyphenyl)piperazine (assumed) (1.0 g, 4.6 mmol, 1.0 eq), NaBH₃CN (1.5 g, 23.1 mmol, 5.0 eq), ZnCl₂ (3.2 g, 23.1 mmol, 5.0 eq) and MeOH (30 mL). The resulting solution was stirred for 16 hours at 70° C. The resulting mixture was then quenched by the addition of water (20 mL), and then extracted with dichloromethane/methanol=10:1 (2×100 mL). The combined organic layers was washed with brine (2×100 mL), dried over anhydrous Na₂SO₄. After filtration, the filtrate was concentrated under vacuum. The residue was purified by Prep-TLC (dichloromethane/methanol=10:1) to afford (3R)-1-(6,8-difluoro-3,4-dihydro-2H-1-benzopyran-3-yl)-3-(2-isopropoxyphenyl)piperazine (145 mg, 8.1%) as a colorless oil. LC-MS (ES, m/z) M+1: 389.

Synthesis of (3R)-1-[(3S)-6,8-difluoro-3,4-dihydro-2H-1-benzopyran-3-yl]-3-(2-isopropoxyphenyl)piperazine (assumed) and (3R)-1-[(3R)-6,8-difluoro-3,4-dihydro-2H-1-benzopyran-3-yl]-3-(2-isopropoxyphenyl)piperazine (assumed): The crude product (3R)-1-(6,8-difluoro-3,4-dihydro-2H-1-benzopyran-3-yl)-3-(2-isopropoxyphenyl)piperazine (145 mg, 0.373 mmol, 1 eq) was purified by Prep-CHIRAL-SFC using the following conditions to give (3R)-1-[(3S)-6,8-difluoro-3,4-dihydro-2H-1-benzopyran-3-yl]-3-(2-isopropoxyphenyl)piperazine (assumed) (50 mg, 34.5%) and (3R)-1-[(3R)-6,8-difluoro-3,4-dihydro-2H-1-benzopyran-3-yl]-3-(2-isopropoxyphenyl)piperazine (50 mg, 34.5%) (assumed) a colorless oil. (3R)-1-[(3S)-6,8-difluoro-3,4-dihydro-2H-1-benzopyran-3-yl]-3-(2-isopropoxyphenyl)piperazine (assumed): RT1=5.7 min in Lux 5 um Cellulose-4, 3×25 cm, 5 μm; Mobile Phase A: CO₂, Mobile Phase B: MeOH (0.1% 2 M NH₃-MeOH); Flow rate: 100 mL/min; Gradient: isocratic 40% B; wave length: 220 nm; Sample Solvent: MeOH: MeCN=2:1; Injection Volume: 1.5 mL; Number of Runs: 25. LC-MS (ES, m/z) M+1: 389. (3R)-1-[(3R)-6,8-difluoro-3,4-dihydro-2H-1-benzopyran-3-yl]-3-(2-isopropoxyphenyl)piperazine (assumed): RT2=7.2 min in Lux 5 um Cellulose-4, 3×25 cm, 5 μm; Mobile Phase A: CO₂, Mobile Phase B: MeOH (0.1% 2 M NH₃-MeOH); Flow rate: 100 mL/min; Gradient: isocratic 40% B; wave length: 220 nm; Sample Solvent: MeOH: MeCN=2:1; Injection Volume: 1.5 mL; Number of Runs: 25. LC-MS (ES, m/z) M+1: 389.

Synthesis of 4-(2-((R)-4-((S)-6,8-difluorochroman-3-yl)-2-(2-isopropoxyphenyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide (assumed): Into a 2 mL sealed-tube, were added (3R)-1-[(3S)-6,8-difluoro-3,4-dihydro-2H-1-benzopyran-3-yl]-3-(2-isopropoxyphenyl)piperazine (assumed) (10 mg, 0.03 mmol, 1.0 eq), 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-N-[(3R)-5-nitro-3-(oxan-4-yl)-3,4-dihydro-2H-1,4-benzoxazin-7-ylsulfonyl]-4-{2-oxo-7-azaspiro[3.5]nonan-7-yl}benzamide (20 mg, 0.03 mmol, 1.0 eq), NaBH₃CN (8 mg, 0.1 mmol, 5.0 eq), ZnCl₂ (17 mg, 0.1 mmol, 5.0 eq) and MeOH (0.5 mL). The resulting solution was stirred for 16 hours at 70° C. The resulting mixture was then quenched by the addition of water (10 mL), and then extracted with dichloromethane/methanol=10:1 (2×10 mL). The combined organic layers was washed with brine (2×10 mL) and dried over anhydrous Na₂SO₄. After filtration, the filtrate was concentrated under vacuum. The crude product was purified by Prep-HPLC using the following conditions (Column: XBridge Prep OBD C18 Column, 30×150 mm, 5 μm; Mobile Phase A: water (0.05% NH₃·H₂O), Mobile Phase B: MeCN; Flow rate: 60 mL/min; Gradient: 25% B to 55% B in 8 min, 55% B; Wave Length: 254/220 nm; RT1(min): 6.7) to afford 4-(2-((R)-4-((S)-6,8-difluorochroman-3-yl)-2-(2-isopropoxyphenyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide (assumed) as a light yellow solid (9 mg, 30.9%). LC-MS (ES, m/z) M+1: 1186. ¹H NMR (300 MHz, DMSO-d₆) δ 12.71 (s, 1H), 11.09 (s, 1H), 8.79-8.76 (m, 1H), 7.96-7.92 (m, 1H), 7.73-7.68 (m, 1H), 7.41-7.38 (m, 1H), 7.19-7.15 (m, 1H), 7.07-7.04 (m, 2H), 7.00-6.91 (m, 3H), 6.86-6.80 (m, 2H), 6.75-6.70 (m, 1H), 6.44-6.40 (m, 1H), 5.99-5.93 (m, 1H), 4.61-4.55 (m, 2H), 4.28-4.20 (m, 3H), 4.01-3.95 (m, 4H), 3.90-3.46 (m, 9H), 3.25-2.55 (m, 12H), 2.35-2.27 (m, 1H), 1.77-1.75 (m, 3H), 1.61-1.57 (m, 3H), 1.47-1.14 (m, 13H), 1.12-1.08 (m, 1H), 0.89-0.85 (m, 1H).

### Example 221 Preparation of 4-(2-((R)-4-((R)-6,8-difluorochroman-3-yl)-2-(2-isopropoxyphenyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide (assumed)

Synthesis of 4-(2-((R)-4-((R)-6,8-difluorochroman-3-yl)-2-(2-isopropoxyphenyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide (assumed): Into a 2 mL sealed-tube, were added (3R)-1-[(3R)-6,8-difluoro-3,4-dihydro-2H-1-benzopyran-3-yl]-3-(2-isopropoxyphenyl)piperazine (assumed) (10 mg, 0.03 mmol, 1.0 eq), 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-N-[(3R)-5-nitro-3-(oxan-4-yl)-3,4-dihydro-2H-1,4-benzoxazin-7-ylsulfonyl]-4-{2-oxo-7-azaspiro[3.5]nonan-7-yl}benzamide (20 mg, 0.03 mmol, 1.0 eq), NaBH₃CN (8 mg, 0.1 mmol, 5.0 eq), ZnCl₂ (17 mg, 0.1 mmol, 5.0 eq) and MeOH (0.5 mL). The resulting solution was stirred for 16 hours at 70° C. The resulting mixture was then quenched by the addition of water (1 mL). The resulting mixture was extracted with dichloromethane/methanol=10:1 (2×10 mL) and washed with brine (2×10 mL) and the organic layers combined. The mixture was dried over anhydrous Na₂SO₄. After filtration, the filtrate was concentrated under vacuum. The crude product was purified by Prep-HPLC using the following conditions (Column: XBridge Prep OBD C18 Column, 30×150 mm, 5 μm; Mobile Phase A: water (0.05% NH₃·H₂O), Mobile Phase B: MeCN; Flow rate: 60 mL/min; Gradient: 25% B to 55% B in 8 min, 55% B; Wave Length: 254/220 nm; RT1(min): 6.7) to afford 4-(2-((R)-4-((R)-6,8-difluorochroman-3-yl)-2-(2-isopropoxyphenyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide (assumed) as a light yellow solid (9 mg, 30.9%). LC-MS (ES, m/z) M+1: 1186. ¹H NMR (300 MHz, DMSO-d₆) δ 12.70 (s, 1H), 11.08 (s, 1H), 8.78-8.75 (m, 1H), 7.95-7.93 (m, 1H), 7.70-7.65 (m, 1H), 7.40-7.38 (m, 1H), 7.17-7.13 (m, 1H), 7.08-7.02 (m, 2H), 7.03-6.90 (m, 3H), 6.86-6.77 (m, 2H), 6.75-6.70 (m, 1H), 6.45-6.38 (m, 1H), 5.96-5.90 (m, 1H), 4.60-4.55 (m, 2H), 4.29-4.20 (m, 3H), 4.05-3.95 (m, 4H), 3.92-3.42 (m, 9H), 3.28-2.55 (m, 12H), 2.38-2.27 (m, 1H), 1.78-1.76 (m, 3H), 1.62-1.58 (m, 3H), 1.48-1.13 (m, 13H), 1.13-1.07 (m, 1H), 0.88-0.85 (m, 1H).

### Example 222 Preparation of 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-4-{2-[(2R)-2-(2-isopropoxyphenyl)-4-phenylpiperazin-1-yl]-7-azaspiro[3.5]nonan-7-yl}-N-[(3R)-5-nitro-3-(oxan-4-yl)-3,4-dihydro-2H-1,4-benzoxazin-7-ylsulfonyl]benzamide

Synthesis of (3R)-3-(2-isopropoxyphenyl)-1-phenylpiperazine: Into an 8 mL sealed tube, were placed bromobenzene (200 mg, 1.3 mmol, 1.0 eq), (2R)-2-(2-isopropoxyphenyl)piperazine (281 mg, 1.3 mmol, 1.0 eq), t-BuOK (429 mg, 3.8 mmol, 3.0 eq), Pd₂(dba)₃ (117 mg, 0.1 mmol, 0.1 eq), A-Phos (20 mg, 10%), toluene (2 mL). The resulting solution was stirred for overnight at 90° C. under nitrogen atmosphere. The resulting mixture was then quenched by the addition of water (30 mL). The resulting solution was extracted with dichloromethane (2×30 mL) and washed with brine (2×30 mL) and the organic layers combined. The mixture was dried over anhydrous Na₂SO₄. After filtration, the filtrate was concentrated under vacuum. The crude residue was applied onto a silica gel column and eluted with dichloromethane/methanol=10:1 to give (3R)-3-(2-isopropoxyphenyl)-1-phenylpiperazine as a light yellow solid (100 mg, 26.5%). LC-MS (ESI, m/z) M+1: 297.

Synthesis of 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-4-{2-[(2R)-2-(2-isopropoxyphenyl)-4-phenylpiperazin-1-yl]-7-azaspiro[3.5]nonan-7-yl}-N-[(3R)-5-nitro-3-(oxan-4-yl)-3,4-dihydro-2H-1,4-benzoxazin-7-ylsulfonyl]benzamide: Into an 8-mL sealed tube, were placed 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-N-[(3R)-5-nitro-3-(oxan-4-yl)-3,4-dihydro-2H-1,4-benzoxazin-7-ylsulfonyl]-4-{2-oxo-7-azaspiro[3.5]nonan-7-yl}benzamide (20 mg, 0.02 mmol, 1.0 eq), (3R)-3-(2-isopropoxyphenyl)-1-phenylpiperazine (9 mg, 0.03 mmol, 1.2 eq), ZnCl₂ (10 mg, 0.1 mmol, 3.0 eq), NaBH₃CN (5 mg, 0.08 mmol, 3.0 eq), MeOH (0.5 mL). The resulting solution was stirred for 4 hours at 70° C. in an oil bath. The crude product was purified by Flash-Prep-HPLC using the following conditions: Column: Xselect CSH C18 OBD Column 30*150 mm 5 μm, n; Mobile Phase A: water (0.1% FA), Mobile Phase B: CH₃CN; Flow rate: 60 mL/min; Gradient: 35% B to 55% B in 8 min, 55% B; Wave Length: 220/254 nm; RT1(min): 6.52; Number Of Runs: 3. Finally, 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-4-{2-[(2R)-2-(2-isopropoxyphenyl)-4-phenylpiperazin-1-yl]-7-azaspiro[3.5]nonan-7-yl}-N-[(3R)-5-nitro-3-(oxan-4-yl)-3,4-dihydro-2H-1,4-benzoxazin-7-ylsulfonyl]benzamide was obtains as a yellow solid (5 mg, 18.6%). LC-MS (ESI, m/z) M+1: 1094. ¹H NMR (400 MHz, Methanol-d₄) δ 8.04-8.00 (m, 1H), 7.90-7.83 (m, 1H), 7.55-7.48 (m, 1H), 7.32-7.20 (m, 4H), 7.06-6.90 (m, 6H), 6.90-6.81 (m, 1H), 6.74-6.65 (m, 1H), 6.53-6.48 (m, 1H), 6.03-5.96 (m, 1H), 4.74-4.67 (m, 1H), 4.33-4.21 (m, 4H), 4.17-4.09 (m, 1H), 4.04-3.89 (m, 2H), 3.82-3.64 (m, 1H), 3.51-3.39 (m, 6H), 3.30-3.07 (m, 6H), 3.06-2.95 (m, 2H), 2.64-2.54 (m, 1H), 2.06-1.97 (m, 1H), 1.90-1.64 (m, 6H), 1.62-1.42 (m, 9H), 1.40-1.35 (m, 6H), 1.33-1.22 (m, 1H).

### Example 223 Preparation of 4-(2-((R)-2-(2-isopropoxyphenyl)-4-(pyridin-4-yl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide (assumed)

Synthesis of (3R)-3-(2-isopropoxyphenyl)-1-(pyridin-4-yl)piperazine (assumed): Into a 40 mL sealed tube were added 4-bromopyridine (200 mg, 1.3 mmol, 1.0 eq), (2R)-2-(2-isopropoxyphenyl)piperazine (279 mg, 1.3 mmol, 1.0 eq), Pd₂(dba)₃ (116 mg, 0.1 mmol, 0.1 eq), 4-(di-tert-butylphosphanyl)-N,N-dimethylaniline (180 mg, 0.3 mmol, 0.2 eq), t-BuOK (426 mg, 3.8 mmol, 3.0 eq) and toluene (4 mL) at 25° C. The resulting mixture was stirred for 5 hours at 90° C. under nitrogen atmosphere. The reaction was quenched by the addition of water (5 mL) and then extracted with EtOAc (2×20 mL). The combined organic layers were washed with brine (20 mL), dried over anhydrous Na₂SO₄. After filtration, the filtrate was concentrated under reduced pressure. The residue was purified by Prep-TLC (CH₂Cl₂/MeOH=10:1) to afford (3R)-3-(2-isopropoxyphenyl)-1-(pyridin-4-yl)piperazine (assumed) as a colorless oil (160 mg, 42.5%). LC-MS (ES, m/z) M+1: 298.

Synthesis of 4-(2-((R)-2-(2-isopropoxyphenyl)-4-(pyridin-4-yl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide (assumed): Into a 2-mL sealed-tube, were added (3R)-3-(2-isopropoxyphenyl)-1-(pyridin-4-yl)piperazine (assumed) (7 mg, 0.03 mmol, 1.0 eq), 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-N-[(3R)-5-nitro-3-(oxan-4-yl)-3,4-dihydro-2H-1,4-benzoxazin-7-ylsulfonyl]-4-{2-oxo-7-azaspiro[3.5]nonan-7-yl}benzamide (20 mg, 0.03 mmol, 1.0 eq), NaBH₃CN (8 mg, 0.1 mmol, 5.0 eq), ZnCl₂ (17 mg, 0.1 mmol, 5.0 eq) and MeOH (0.5 mL). The resulting mixture was stirred for 16 hours at 70° C. The resulting solution was diluted with dichloromethane/methanol=10:1 (2×10 mL). The combined organic layer was washed with brine (2×10 mL) and dried over anhydrous Na₂SO₄. After filtration, the filtrate was concentrated under vacuum. The crude product was purified by Prep-HPLC using the following conditions (Column: XBridge Prep OBD C18 Column, 30×150 mm, 5 μm; Mobile Phase A: water (0.05% NH₃·H₂O), Mobile Phase B: MeCN; Flow rate: 60 mL/min; Gradient: 25% B to 55% B in 8 min, 55% B; wave Length: 254/220 nm) to afford 4-(2-((R)-2-(2-isopropoxyphenyl)-4-(pyridin-4-yl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide (assumed) as a light yellow solid (8 mg, 29.7%). LC-MS (ES, m/z) M+1: 1096. ¹H NMR (400 MHz, DMSO-d₆) δ 11.03 (s, 1H), 8.72-8.70 (m, 1H), 8.23-8.12 (m, 2H), 8.09-8.00 (m, 1H), 7.94-7.90 (m, 1H), 7.62-7.58 (m, 1H), 7.45-7.40 (m, 1H), 7.22-7.18 (m, 2H), 7.11-6.85 (m, 4H), 6.79-6.75 (m, 1H), 6.71-6.68 (m, 1H), 6.42-6.38 (m, 1H), 6.02-5.95 (m, 1H), 4.71-4.60 (m, 2H), 4.37-3.59 (m, 13H), 3.21-2.87 (m, 11H), 2.10-1.96 (m, 2H), 1.82-1.72 (m, 2H), 1.61-1.58 (m, 4H), 1.47-1.05 (m, 12H), 0.86-0.80 (m, 1H).

### Example 224 Preparation of 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-4-{2-[(2R)-2-(2-isopropoxyphenyl)-4-(pyridin-3-yl)piperazin-1-yl]-7-azaspiro[3.5]nonan-7-yl}-N-[(3R)-5-nitro-3-(oxan-4-yl)-3,4-dihydro-2H-1,4-benzoxazin-7-ylsulfonyl]benzamide

Synthesis of (3R)-3-(2-isopropoxyphenyl)-1-(pyridin-3-yl)piperazine: Into a 40 mL sealed tube were added 3-iodopyridine (200 mg, 1.0 mmol, 1.0 eq), (2R)-2-(2-isopropoxyphenyl)piperazine (215 mg, 1.0 mmol, 1.0 eq), Pd₂(dba)₃ (89 mg, 0.1 mmol, 0.1 eq), A-Phos (20 mg, 10%), t-BuOK (328 mg, 2.9 mmol, 3.0 eq) and toluene (4 mL) at 25° C. The resulting mixture was stirred for 5 hours at 90° C. under nitrogen atmosphere. The resulting mixture was then quenched by the addition of water (30 mL). The resulting solution was extracted with dichloromethane (2×30 mL) and washed with brine (2×30 mL) and the organic layers combined. The mixture was dried over anhydrous Na₂SO₄. After filtration, the filtrate was concentrated under vacuum. The crude residue was applied onto a silica gel column and eluted with dichloromethane/methanol=10:1 to give (3R)-3-(2-isopropoxyphenyl)-1-(pyridin-3-yl)piperazine as a colorless oil (160 mg, 55.1%). LC-MS (ES, m/z) M+1: 298.

Synthesis of 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-4-{2-[(2R)-2-(2-isopropoxyphenyl)-4-(pyridin-3-yl)piperazin-1-yl]-7-azaspiro[3.5]nonan-7-yl}-N-[(3R)-5-nitro-3-(oxan-4-yl)-3,4-dihydro-2H-1,4-benzoxazin-7-ylsulfonyl]benzamide: Into an 8-mL sealed tube, were placed 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-N-[(3R)-5-nitro-3-(oxan-4-yl)-3,4-dihydro-2H-1,4-benzoxazin-7-ylsulfonyl]-4-{2-oxo-7-azaspiro[3.5]nonan-7-yl}benzamide (20 mg, 0.03 mmol, 1.0 eq), (3R)-3-(2-isopropoxyphenyl)-1-(pyridin-3-yl)piperazine (9 mg, 0.03 mmol, 1.2 eq), ZnCl₂ (10 mg, 0.08 mmol, 3.0 eq), NaBH₃CN (5 mg, 0.08 mmol, 3.0 eq), MeOH (0.5 mL). The resulting solution was stirred for 4 hours at 70° C. in an oil bath. The crude product was purified by Flash-Prep-HPLC using the following conditions: Column: Xselect CSH C18 OBD Column 30*150 mm 5 μm, n; Mobile Phase A: water (0.1% FA), Mobile Phase B: CH₃CN; Flow rate: 60 mL/min; Gradient: 20% B to 40% B in 8 min, 40% B; Wave Length: 220/254 nm; RT1(min): 7.32; Number Of Runs: 5. Finally, 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-4-{2-[(2R)-2-(2-isopropoxyphenyl)-4-(pyridin-3-yl)piperazin-1-yl]-7-azaspiro[3.5]nonan-7-yl}-N-[(3R)-5-nitro-3-(oxan-4-yl)-3,4-dihydro-2H-1,4-benzoxazin-7-ylsulfonyl]benzamide was obtains as a yellow solid (5 mg, 18.6%). LC-MS (ESI, m/z) M+1: 1095. ¹H NMR (400 MHz, Chloroform-d) δ 8.88-8.59 (m, 1H), 8.58-8.40 (m, 2H), 8.35-8.29 (m, 2H), 8.17-8.06 (m, 1H), 7.52-7.43 (m, 2H), 7.27-7.07 (m, 2H), 6.95-6.82 (m, 2H), 6.73-6.60 (m, 3H), 6.47-6.09 (m, 3H), 4.66-4.40 (m, 2H), 4.15-3.93 (m, 4H), 3.75-3.68 (m, 2H), 3.54-3.31 (m, 9H), 3.17-2.94 (m, 9H), 2.93-2.85 (m, 2H), 2.42-2.16 (m, 4H), 2.07-2.00 (m, 2H), 1.92-1.58 (m, 4H), 1.56-1.42 (m, 4H), 1.39-1.32 (m, 4H), 1.30-1.17 (m, 1H).

### Example 225 Preparation of 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-4-{2-[(2R)-2-(2-isopropoxyphenyl)-4-(pyridin-2-yl)piperazin-1-yl]-7-azaspiro[3.5]nonan-7-yl}-N-[(3R)-5-nitro-3-(oxan-4-yl)-3,4-dihydro-2H-1,4-benzoxazin-7-ylsulfonyl]benzamide

Synthesis of (3R)-3-(2-isopropoxyphenyl)-1-(pyridin-2-yl)piperazine: Into an 8-mL sealed tube, were placed 2-bromopyridine (200 mg, 1.3 mmol, 1.0 eq), (2R)-2-(2-isopropoxyphenyl)piperazine (279 mg, 1.3 mmol, 1.0 eq), t-BuOK (426 mg, 3.8 mmol, 3.0 eq), Pd₂(dba)₃ (116 mg, 0.1 mmol, 0.1 eq), A-Phos (20 mg, 10%), toluene (2 mL). The resulting solution was stirred for 2.5 hours at 90° C. under nitrogen atmosphere. The resulting mixture was then quenched by the addition of water (30 mL) and extracted with dichloromethane (2×30 mL). The combined organic layers was washed with brine (2×30 mL) and dried over anhydrous Na₂SO₄. After filtration, the filtrate was concentrated under vacuum. The crude residue was applied onto a silica gel column and eluted with dichloromethane/methanol=10:1 to give (3R)-3-(2-isopropoxyphenyl)-1-(pyridin-2-yl)piperazine as a white solid (130 mg, 34.5%). LC-MS (ESI, m/z) M+1: 298.

Synthesis of 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-4-{2-[(2R)-2-(2-isopropoxyphenyl)-4-(pyridin-2-yl)piperazin-1-yl]-7-azaspiro[3.5]nonan-7-yl}-N-[(3R)-5-nitro-3-(oxan-4-yl)-3,4-dihydro-2H-1,4-benzoxazin-7-ylsulfonyl]benzamide: Into an 8-mL sealed tube, were placed 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-N-[(3R)-5-nitro-3-(oxan-4-yl)-3,4-dihydro-2H-1,4-benzoxazin-7-ylsulfonyl]-4-{2-oxo-7-azaspiro[3.5]nonan-7-yl}benzamide (20 mg, 0.03 mmol, 1.0 eq), (3R)-3-(2-isopropoxyphenyl)-1-(pyridin-2-yl)piperazine (9 mg, 0.03 mmol, 1.2 eq), ZnCl₂ (10 mg, 0.08 mmol, 3.0 eq), NaBH₃CN (5 mg, 0.08 mmol, 3.0 eq), MeOH (0.5 mL). The resulting solution was stirred for 4 hours at 70° C. in an oil bath. The crude product was purified by Flash-Prep-HPLC using the following conditions: Column: Xselect CSH C18 OBD Column 30*150 mm 5 μm, n; Mobile Phase A: water (0.1% FA), Mobile Phase B: CH₃CN; Flow rate: 60 mL/min; Gradient: 30% B to 50% B in 8 min, 50% B; Wave Length: 220/254 nm; RT1(min): 6.80; Number Of Runs: 3. Finally, 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-4-{2-[(2R)-2-(2-isopropoxyphenyl)-4-(pyridin-2-yl)piperazin-1-yl]-7-azaspiro[3.5]nonan-7-yl}-N-[(3R)-5-nitro-3-(oxan-4-yl)-3,4-dihydro-2H-1,4-benzoxazin-7-ylsulfonyl]benzamide was obtains as a yellow solid (5 mg, 18.6%). LC-MS (ESI, m/z) M+1: 1095. ¹H NMR (400 MHz, Methanol-d₄) δ 8.16-7.99 (m, 2H), 7.88-7.83 (m, 1H), 7.62-7.48 (m, 2H), 7.32-7.20 (m, 2H), 7.06-6.90 (m, 4H), 6.86-6.80 (m, 1H), 6.75-6.66 (m, 2H), 6.56-6.47 (m, 1H), 6.02-5.95 (m, 1H), 4.72-4.65 (m, 1H), 4.34-4.20 (m, 4H), 4.17-4.06 (m, 2H), 4.04-3.88 (m, 4H), 3.73-3.63 (m, 1H), 3.56-3.38 (m, 4H), 3.25-3.10 (m, 8H), 2.46-2.41 (m, 1H), 2.02-1.83 (m, 2H), 1.80-1.65 (m, 4H), 1.61-1.38 (m, 8H), 1.37-1.33 (m, 6H), 1.31-1.23 (m, 1H).

### Example 226 Preparation of 4-{2-[(2R)-4-(4,4-dimethylcyclohexyl)-2-(2-isopropoxyphenyl)piperazin-1-yl]-7-azaspiro[3.5]nonan-7-yl}-2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-N-[(3R)-5-nitro-3-(oxan-4-yl)-3,4-dihydro-2H-1,4-benzoxazin-7-ylsulfonyl]benzamide

Synthesis of (3R)-1-(4,4-dimethylcyclohexyl)-3-(2-isopropoxyphenyl)piperazine: Into an 8-mL sealed tube, were placed 4,4-dimethylcyclohexan-1-one (200 mg, 1.6 mmol, 1.0 eq), (2R)-2-(2-isopropoxyphenyl)piperazine (419 mg, 1.9 mmol, 1.2 eq), ZnCl₂ (648 mg, 4.8 mmol, 3.0 eq), NaBH₃CN (299 mg, 4.8 mmol, 3.0 eq), MeOH (2 mL). The resulting solution was stirred for 2 hours at 25° C. The resulting mixture was then quenched by the addition of water (30 mL), and then extracted with dichloromethane (2×30 mL). The combined organic layers was washed with brine (2×30 mL), dried over anhydrous Na₂SO₄. After filtration, the filtrate was concentrated under vacuum. The crude residue was applied onto a silica gel column and eluted with dichloromethane/methanol=10:1 to give (3R)-1-(4,4-dimethylcyclohexyl)-3-(2-isopropoxyphenyl)piperazine as a light yellow solid (100 mg, 19.1%). LC-MS (ES, m/z) M+1: 331.

Synthesis of 4-{2-[(2R)-4-(4,4-dimethylcyclohexyl)-2-(2-isopropoxyphenyl)piperazin-1-yl]-7-azaspiro[3.5]nonan-7-yl}-2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-N-[(3R)-5-nitro-3-(oxan-4-yl)-3,4-dihydro-2H-1,4-benzoxazin-7-ylsulfonyl]benzamide: Into an 8-mL sealed tube, were placed 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-N-[(3R)-5-nitro-3-(oxan-4-yl)-3,4-dihydro-2H-1,4-benzoxazin-7-ylsulfonyl]-4-{2-oxo-7-azaspiro[3.5]nonan-7-yl}benzamide (20 mg, 0.02 mmol, 1.0 eq), (3R)-1-(4,4-dimethylcyclohexyl)-3-(2-isopropoxyphenyl)piperazine (10 mg, 0.03 mmol, 1.2 eq), ZnCl₂ (10 mg, 0.08 mmol, 3.0 eq), NaBH₃CN (5 mg, 0.08 mmol, 3.0 eq), MeOH (0.5 mL). The resulting solution was stirred for 4 hours at 70° C. in an oil bath. The crude product was purified by Flash-Prep-HPLC using the following conditions: Column: XBridge Shield RP18 OBD Column, 30*150 mm, 5 μm; Mobile Phase A: water (0.05% NH₃·H₂O), Mobile Phase B: CH₃CN; Flow rate: 60 mL/min; Gradient: 25% B to 45% B in 8 min, 45% B; Wave Length: 254/220 nm; RT1(min): 6.80; Number Of Runs: 2. Finally, 4-{2-[(2R)-4-(4,4-dimethylcyclohexyl)-2-(2-isopropoxyphenyl)piperazin-1-yl]-7-azaspiro[3.5]nonan-7-yl}-2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-N-[(3R)-5-nitro-3-(oxan-4-yl)-3,4-dihydro-2H-1,4-benzoxazin-7-ylsulfonyl]benzamide was obtained as a yellow solid (6 mg, 21.6%). LC-MS (ES, m/z) M+1:1129. ¹H NMR (400 MHz, Methanol-d₄) δ 8.05-8.02 (m, 1H), 7.78-7.73 (m, 1H), 7.44-7.37 (m, 1H), 7.36-7.20 (m, 2H), 7.02-6.89 (m, 4H), 6.69-6.52 (m, 2H), 6.05-5.95 (m, 1H), 4.71-4.64 (m, 1H), 4.33-4.05 (m, 4H), 4.04-3.76 (m, 4H), 3.64-3.53 (m, 1H), 3.52-3.38 (m, 4H), 3.25-2.85 (m, 10H), 2.75-2.58 (m, 2H), 2.43-2.30 (m, 1H), 1.95-1.56 (m, 10H), 1.55-1.39 (m, 10H), 1.37-1.33 (m, 6H), 1.30-1.16 (m, 2H), 0.97-0.89 (m, 6H), 0.87-0.82 (m, 1H).

### Example 227 Preparation of 4-{2-[(2R)-4-[(3,4-difluorophenyl)methyl]-2-(2-isopropoxyphenyl)piperazin-1-yl]-7-azaspiro[3.5]nonan-7-yl}-2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-5-fluoro-N-[(3R)-5-nitro-3-(oxan-4-yl)-3,4-dihydro-2H-1,4-benzoxazin-7-ylsulfonyl]benzamide

Synthesis of methyl 2-bromo-5-fluoro-4-(2-oxo-7-azaspiro[3.5]nonan-7-yl)benzoate: Into a 500 mL 3-necked round-bottom flask, were placed 7-azaspiro[3.5]nonan-2-one hydrochloride (7.3 g, 41.6 mmol, 1.0 eq), N,N-dimethylformamide (100 mL), Cs₂CO₃ (33.8 g, 103.9 mmol, 2.5 eq), methyl 2-bromo-4,5-difluorobenzoate (10.5 g, 45.1 mmol, 1.1 eq). The reaction mixture was stirred for 8 hours at 80° C. The resulting mixture was quenched by the addition of water (200 mL) and extracted with EtOAc (3×200 mL). The combined organic layer was washed with brine (300 mL), and dried over anhydrous Na₂SO₄. After filtration, the filtrate was concentrated under vacuum. The crude residue was applied onto a silica gel column and eluted with ethyl acetate/petroleum ether=1:2 to give methyl 2-bromo-5-fluoro-4-(2-oxo-7-azaspiro[3.5]nonan-7-yl)benzoate as a light yellow solid (6.9 g, 46.1%). LC-MS (ES, m/z) M+1: 370/372.

Synthesis of 2-bromo-5-fluoro-4-(2-oxo-7-azaspiro[3.5]nonan-7-yl)benzoic acid: Into a 100 mL round-bottom flask, were placed methyl 2-bromo-5-fluoro-4-(2-oxo-7-azaspiro[3.5]nonan-7-yl)benzoate (2.0 g, 5.7 mmol, 1.0 eq), methanol (5 mL), water (5 mL), NaOH (911 mg, 22.8 mmol, 4.0 eq). The reaction mixture was stirred for 24 hours at 50° C. The resulting mixture was then diluted with water (30 mL) and acidified to pH=5 with HCl (1 M). The precipitated solids were collected by filtration and dried under infrared light to give 2-bromo-5-fluoro-4-(2-oxo-7-azaspiro[3.5]nonan-7-yl)benzoic acid as a yellow solid (1.9 g, 98.9%). LC-MS (ES, m/z) M+1: 356/358.

Synthesis of 2-bromo-5-fluoro-N-[(3R)-5-nitro-3-(oxan-4-yl)-3,4-dihydro-2H-1,4-benzoxazin-7-ylsulfonyl]-4-{2-oxo-7-azaspiro[3.5]nonan-7-yl}benzamide: Into a 50 mL round-bottom flask, were placed 2-bromo-5-fluoro-4-{2-oxo-7-azaspiro[3.5]nonan-7-yl}benzoic acid (330 mg, 0.9 mmol, 1.0 eq), EDCl (231 mg, 1.2 mmol, 1.3 eq), DMAP (125 mg, 1.0 mmol, 1.1 eq), DCM (6 mL). The mixture was stirred at 25° C. for 10 minute and a solution of (3R)-5-nitro-3-(oxan-4-yl)-3,4-dihydro-2H-1,4-benzoxazine-7-sulfonamide (350 mg, 1.0 mmol, 1.1 eq), TEA (234 mg, 2.3 mmol, 2.5 eq) in DCM (3.5 mL) was added dropwise at 25° C. Then the mixture was stirred for 16 hours at 25° C. The resulting mixture was then quenched by the addition of water (100 mL) and then extracted with dichloromethane (2×100 mL). The combined organic layer was washed with brine (100 mL), the mixture was dried over anhydrous Na₂SO₄. After filtration, the filtrate was concentrated under vacuum. The crude residue was purified onto a silica gel column and eluted with dichloromethane/methanol=15:1 to give 2-bromo-5-fluoro-N-[(3R)-5-nitro-3-(oxan-4-yl)-3,4-dihydro-2H-1,4-benzoxazin-7-ylsulfonyl]-4-{2-oxo-7-azaspiro[3.5]nonan-7-yl}benzamide as a yellow solid (450 mg, 71.3%). LC-MS (ES, m/z) M+1: 681/683.

Synthesis of 5-fluoro-N-[(3R)-5-nitro-3-(oxan-4-yl)-3,4-dihydro-2H-1,4-benzoxazin-7-ylsulfonyl]-4-{2-oxo-7-azaspiro[3.5]nonan-7-yl}-2-[(3R,8S)-15-{[2-(trimethylsilyl)ethoxy]methyl}-2,5-dioxa-9,15,17-triazatetracyclo[8.7.0.0\hat{ }{3,8}.0\hat{ }{12,16}]heptadeca-1(10),11,13,16-tetraen-9-yl]benzamide: Into a 40 mL sealed-tube, were placed 2-bromo-5-fluoro-N-[(3R)-5-nitro-3-(oxan-4-yl)-3,4-dihydro-2H-1,4-benzoxazin-7-ylsulfonyl]-4-{2-oxo-7-azaspiro[3.5]nonan-7-yl}benzamide (400 mg, 0.6 mmol, 1.0 eq), (3R,8S)-15-{[2-(trimethylsilyl)ethoxy]methyl}-2,5-dioxa-9,15,17-triazatetracyclo[8.7.0.0\hat{ }{3,8}.0\hat{ }{12,16}]heptadeca-1(10),11,13,16-tetraene (212 mg, 0.6 mmol, 1.0 eq), DMSO (4 mL), Cul (67 mg, 0.4 mmol, 0.6 eq), N,N′-diphenyl-ethanediamide (85 mg, 0.4 mmol, 0.6 eq). The resulting mixture was stirred for 2 hours at 100° C. under nitrogen atmosphere. The resulting solution was extracted with dichloromethane/methanol=20:1 (2×100 mL) and washed with brine (2×100 mL) and the organic layers combined. The mixture was dried over anhydrous Na₂SO₄. After filtration, the filtrate was concentrated under vacuum. The crude residue was purified onto a silica gel column and eluted with dichloromethane/methanol=20:1 to give 5-fluoro-N-[(3R)-5-nitro-3-(oxan-4-yl)-3,4-dihydro-2H-1,4-benzoxazin-7-ylsulfonyl]-4-{2-oxo-7-azaspiro[3.5]nonan-7-yl}-2-[(3R,8S)-15-{[2-(trimethylsilyl)ethoxy]methyl}-2,5-dioxa-9,15,17-triazatetracyclo[8.7.0.0\hat{ }{3,8}.0\hat{ }{12,16}]heptadeca-1(10),11,13,16-tetraen-9-yl]benzamide as a yellow solid (250 mg, 44.3%). LC-MS (ES, m/z) M+1: 962.

Synthesis of 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-5-fluoro-N-[(3R)-5-nitro-3-(oxan-4-yl)-3,4-dihydro-2H-1,4-benzoxazin-7-ylsulfonyl]-4-{2-oxo-7-azaspiro[3.5]nonan-7-yl}benzamide: Into a 20 mL sealed-tube, were placed 5-fluoro-N-[(3R)-5-nitro-3-(oxan-4-yl)-3,4-dihydro-2H-1,4-benzoxazin-7-ylsulfonyl]-4-{2-oxo-7-azaspiro[3.5]nonan-7-yl}-2-[(3R,8S)-15-{[2-(trimethylsilyl)ethoxy]methyl}-2,5-dioxa-9,15,17-triazatetracyclo[8.7.0.0\hat{ }{3,8}.0\hat{ }{12,16}]heptadeca-1(10),11,13,16-tetraen-9-yl]benzamide (200 mg, 0.2 mmol, 1.0 eq), ethylenediamine (250 mg, 4.2 mmol, 20.0 eq), TBAF in THE (4 mL). The resulting mixture was stirred for 16 hours at 80° C. The reaction was quenched by the addition of water (100 mL), and then extracted with DCM (2×100 mL). The combined organic layers were washed with brine (100 mL), dried over anhydrous Na₂SO₄. After filtration, the filtrate was concentrated under vacuum. The residue was purified by Prep-TLC (dichloromethane/methanol=15:1) to give 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-5-fluoro-N-[(3R)-5-nitro-3-(oxan-4-yl)-3,4-dihydro-2H-1,4-benzoxazin-7-ylsulfonyl]-4-{2-oxo-7-azaspiro[3.5]nonan-7-yl}benzamide as a yellow solid (80 mg, 46.3%). LC-MS (ES, m/z) M+1: 832.

Synthesis of 4-{2-[(2R)-4-[(3,4-difluorophenyl)methyl]-2-(2-isopropoxyphenyl)piperazin-1-yl]-7-azaspiro[3.5]nonan-7-yl}-2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-5-fluoro-N-[(3R)-5-nitro-3-(oxan-4-yl)-3,4-dihydro-2H-1,4-benzoxazin-7-ylsulfonyl]benzamide: Into an 8-mL sealed-tube, were placed 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-5-fluoro-N-[(3R)-5-nitro-3-(oxan-4-yl)-3,4-dihydro-2H-1,4-benzoxazin-7-ylsulfonyl]-4-{2-oxo-7-azaspiro[3.5]nonan-7-yl}benzamide (25 mg, 0.03 mmol, 1.0 eq), (3R)-1-[(3,4-difluorophenyl)methyl]-3-(2-isopropoxyphenyl)piperazine (14 mg, 0.04 mmol, 1.3 eq), MeOH (1 mL), ZnCl₂ (12 mg, 0.09 mmol, 3.0 eq), NaBH₃CN (6 mg, 0.09 mmol, 3.0 eq). The resulting solution was stirred for 4 hours at 70° C. The resulting mixture was then quenched by the addition of water (20 mL), and then extracted with dichloromethane/methanol=10:1 (2×30 mL). The combined organic layers was washed with brine (2×20 mL) and dried over anhydrous Na₂SO₄. After filtration, the filtrate was concentrated under vacuum. The crude product was purified by Prep-HPLC using the following conditions: Column, SunFire Prep C18 OBD Column, 50*250 mm 5 μm 10 nm; mobile phase, water (0.05% FA) and CH₃CN (40% Phase B up to 50% in 7 min); Detector, UV 254/220 nm. Finally, 4-{2-[(2R)-4-[(3,4-difluorophenyl)methyl]-2-(2-isopropoxyphenyl)piperazin-1-yl]-7-azaspiro[3.5]nonan-7-yl}-2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-5-fluoro-N-[(3R)-5-nitro-3-(oxan-4-yl)-3,4-dihydro-2H-1,4-benzoxazin-7-ylsulfonyl]benzamide was obtained as a yellow solid (10 mg, 28.6%). LC-MS (ES, m/z) M+1: 1162. ¹H NMR (400 MHz, Methanol-d₄) δ 8.66-8.35 (m, 1H), 8.15-8.02 (m, 1H), 7.46-7.36 (m, 2H), 7.34-7.27 (m, 3H), 7.24-7.18 (m, 1H), 7.16-7.11 (m, 1H), 7.052-6.92 (m, 3H), 6.72-6.67 (m, 1H), 6.53 (s, 1H), 6.01-5.98 (m, 1H), 4.71-4.64 (m, 1H), 4.52-4.37 (m, 1H), 4.21-4.17 (m, 3H), 4.11-3.99 (m, 4H), 3.89-3.81 (m, 1H), 3.63-3.59 (m, 2H), 3.49-3.39 (m, 6H), 3.05-2.99 (m, 1H), 2.91-2.74 (m, 6H), 2.54-2.45 (m, 2H), 2.14-2.03 (m, 1H), 1.90-1.70 (m, 5H), 1.62-1.46 (m, 8H), 1.40-1.36 (m, 3H), 1.32-1.29 (m, 4H).

### Example 228 Preparation of 6-{2-[(2R)-4-[(3,4-difluorophenyl)methyl]-2-(2-isopropoxyphenyl)piperazin-1-yl]-7-azaspiro[3.5]nonan-7-yl}-4-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-5-fluoro-N-[(3R)-5-nitro-3-(oxan-4-yl)-3,4-dihydro-2H-1,4-benzoxazin-7-ylsulfonyl]pyridine-3-carboxamide (Rotamer1)

Synthesis of 6-chloro-5-fluoropyridine-3-carboxylic acid: Into a 1000 mL round-bottom flask, were added 2-chloro-3-fluoro-5-methylpyridine (25.0 g, 171.8 mmol, 1.0 eq), Pyridine (100 mL), H₂O (100 mL) and KMnO₄ (162.9 g, 1030.5 mmol, 6.0 eq) at 25° C. The resulting mixture was stirred for 8 hours at 100° C. The resulting mixture was filtered, the filter cake was washed with EtOAc (2×50 mL). The filtrate was concentrated under vacuum. The crude residue was purified onto a silica gel column and eluted with dichloromethane/methanol=10:1 to give 6-chloro-5-fluoropyridine-3-carboxylic acid as a white solid (14.0 g, 46.4%). ¹H NMR (400 MHz, DMSO-d₆) δ 13.89 (s, 1H), 8.75 (d, J=1.9 Hz, 1H), 8.28 (dd, J=8.7, 1.9 Hz, 1H).

Synthesis of 4-bromo-6-chloro-5-fluoropyridine-3-carboxylic acid: Into a 500 mL 3-necked round-bottom flask under nitrogen atmosphere, were placed 6-chloro-5-fluoropyridine-3-carboxylic acid (13.0 g, 74.1 mmol, 1.0 eq), THE (150 mL). This was followed by the addition of LDA (207.4 mmol, 2.8 eq) at −78° C. The resulting mixture was stirred for 3 hours at −78° C. To the above mixture was added DBTCE (50.0 g, 148.2 mmol, 2.0 eq) at −78° C. The resulting mixture was stirred for additional 1 hour at −78° C. The resulting mixture was then quenched by the addition of NH₄Cl (300 mL) and then extracted with ethyl acetate (2×200 mL). The combined organic layers was washed with brine (2×200 mL). The mixture was dried over anhydrous Na₂SO₄. After filtration, the filtrate was concentrated under vacuum. The crude residue was purified onto a silica gel column and eluted with dichloromethane/methanol=10:1 to give 4-bromo-6-chloro-5-fluoropyridine-3-carboxylic acid as a light yellow oil (16.5 g, 87.6%). ¹H NMR (300 MHz, DMSO-d₆) δ 14.18 (s, 1H), 8.62 (d, J=0.8 Hz, 1H).

Synthesis of methyl 4-bromo-6-chloro-5-fluoropyridine-3-carboxylate: Into a 500 mL round-bottom flask, were placed 4-bromo-6-chloro-5-fluoropyridine-3-carboxylic acid (16.5 g, 64.8 mmol, 1.0 eq), H₂SO₄ (1.9 g, 19.5 mmol, 0.3 eq), MeOH (170 mL). The resulting solution was stirred for overnight at 70° C. in an oil bath. The residue was neutralized to pH=9 with saturated Na₂CO₃ (aq.), and then extracted with ethyl acetate (2×200 mL). The combined organic layers was washed with water (2×100 mL) and brine (2×100 mL), and dried over anhydrous Na₂SO₄. After filtration, the filtrate was concentrated under vacuum. The crude residue was applied onto a silica gel column and eluted with ethyl acetate/petroleum ether=1:5 to give methyl 4-bromo-6-chloro-5-fluoropyridine-3-carboxylate as a yellow oil (12.5 g, 71.8%). ¹H NMR (300 MHz, DMSO-d₆) δ 8.61 (d, J=0.8 Hz, 1H), 3.92 (s, 3H).

Synthesis of methyl 4-bromo-5-fluoro-6-{2-oxo-7-azaspiro[3.5]nonan-7-yl}pyridine-3-carboxylate: Into a 500 mL round-bottom flask, were placed methyl 4-bromo-6-chloro-5-fluoropyridine-3-carboxylate (10.0 g, 37.2 mmol, 1.0 eq), 7-azaspiro[3.5]nonan-2-one (5.2 g, 37.2 mmol, 1.0 eq), TEA (11.3 g, 111.7 mmol, 3.0 eq), DMSO (100 mL). The resulting solution was stirred for overnight at 100° C. in an oil bath. The resulting mixture was then quenched by the addition of water (100 mL), and then extracted with ethyl acetate (2×100 mL). The combined organic layers was washed with water (2×100 mL) and brine (2×100 mL), dried over anhydrous Na₂SO₄. After filtration, the filtrate was concentrated under vacuum. The crude residue was applied onto a silica gel column and eluted with ethyl acetate/petroleum ether=1:3 to give methyl 4-bromo-5-fluoro-6-{2-oxo-7-azaspiro[3.5]nonan-7-yl}pyridine-3-carboxylate as a colorless oil (4.4 g, 31.8%). ¹H NMR (400 MHz, DMSO-d₆) δ 8.43 (s, 1H), 3.83 (s, 3H), 3.69-3.60 (m, 4H), 2.87 (s, 4H), 1.82-1.75 (m, 4H).

Synthesis of 4-bromo-5-fluoro-6-{2-oxo-7-azaspiro[3.5]nonan-7-yl}pyridine-3-carboxylic acid: Into a 100 mL round-bottom flask, were placed methyl 4-bromo-5-fluoro-6-{2-oxo-7-azaspiro[3.5]nonan-7-yl}pyridine-3-carboxylate (4.3 g, 11.6 mmol, 1.0 eq), NaOH (4.5 mL, 46.3 mmol, 4.0 eq), dioxane:MeOH=1:1 (45 mL). The reaction mixture was stirred for 16 hours at 30° C. The resulting mixture was then diluted with water (30 mL) and acidified to pH=5 with HCl (1 M). The precipitated solids were collected by filtration and dried under infrared light to give 4-bromo-5-fluoro-6-{2-oxo-7-azaspiro[3.5]nonan-7-yl}pyridine-3-carboxylic acid as a white solid (4.0 g, 96.7%). ¹H NMR (300 MHz, DMSO-d₆) δ 13.11 (s, 1H), 8.44 (s, 1H), 3.67-3.54 (m, 4H), 2.87 (s, 4H), 1.84-1.71 (m, 4H).

Synthesis of 4-bromo-5-fluoro-N-[(3R)-5-nitro-3-(oxan-4-yl)-3,4-dihydro-2H-1,4-benzoxazin-7-ylsulfonyl]-6-{2-oxo-7-azaspiro[3.5]nonan-7-yl}pyridine-3-carboxamide: Into a 50-mL round-bottom flask, were added 4-bromo-5-fluoro-6-{2-oxo-7-azaspiro[3.5]nonan-7-yl}pyridine-3-carboxylic acid (250 mg, 0.7 mmol, 1.0 eq), (3R)-5-nitro-3-(oxan-4-yl)-3,4-dihydro-2H-1,4-benzoxazine-7-sulfonamide (240 mg, 0.7 mmol, 1.0 eq), DMAP (102 mg, 0.8 mmol, 1.2 eq), TEA (177 mg, 1.8 mmol, 2.5 eq), CH₂Cl₂ (10 mL) and EDCl (201 mg, 1.1 mmol, 1.5 eq) at 25° C. The resulting mixture was stirred for 6 hours at 25° C. The resulting mixture was diluted with CH₂Cl₂ (20 mL) and washed with water (3×10 mL), dried over anhydrous Na₂SO₄. After filtration, the filtrate was concentrated under vacuum. The crude residue was purified onto a silica gel column and eluted with CH₂Cl₂/MeOH=30:1 to give 4-bromo-5-fluoro-N-[(3R)-5-nitro-3-(oxan-4-yl)-3,4-dihydro-2H-1,4-benzoxazin-7-ylsulfonyl]-6-{2-oxo-7-azaspiro[3.5]nonan-7-yl}pyridine-3-carboxamide as an yellow solid (450 mg, 94.2%). LC-MS (ES, m/z) M+1: 682/684.

Synthesis of 5-fluoro-N-[(3R)-5-nitro-3-(oxan-4-yl)-3,4-dihydro-2H-1,4-benzoxazin-7-ylsulfonyl]-6-{2-oxo-7-azaspiro[3.5]nonan-7-yl}-4-[(3R,8S)-15-{[2-(trimethylsilyl)ethoxy]methyl}-2,5-dioxa-9,15,17-triazatetracyclo[8.7.0.0\hat{ }{3,8}.0\hat{ }{12,16}]heptadeca-1(10),11,13,16-tetraen-9-yl]pyridine-3-carboxamide: Into a 50 mL round-bottom flask, were added 4-bromo-5-fluoro-N-[(3R)-5-nitro-3-(oxan-4-yl)-3,4-dihydro-2H-1,4-benzoxazin-7-ylsulfonyl]-6-{2-oxo-7-azaspiro[3.5]nonan-7-yl}pyridine-3-carboxamide (450 mg, 0.7 mmol, 1.0 eq), (3R,8S)-15-{[2-(trimethylsilyl)ethoxy]methyl}-2,5-dioxa-9,15,17-triazatetracyclo[8.7.0.0\hat{ }{3,8}.0\hat{ }{12,16}]heptadeca-1(10),11,13,16-tetraene (238 mg, 0.7 mmol, 1.0 eq), Cul (25 mg, 0.14 mmol, 0.2 eq), 1,10-phenanthroline (48 mg, 0.28 mmol, 0.4 eq), K₂CO₃ (273 mg, 2.0 mmol, 3.0 eq) and DMSO (10 mL). The resulting mixture was stirred for 14 hours at 100° C. under nitrogen atmosphere. The precipitated solids were collected by filtration and washed with CH₂Cl₂ (3×10 mL). The resulting mixture was diluted with water (20 mL), and extracted with CH₂Cl₂ (3×20 mL). The combined organic layers were washed with brine (20 mL), dried over anhydrous Na₂SO₄. After filtration, the filtrate was concentrated under vacuum. The crude residue was purified by silica gel column chromatography, eluted with CH₂Cl₂/MeOH=15:1 to give 5-fluoro-N-[(3R)-5-nitro-3-(oxan-4-yl)-3,4-dihydro-2H-1,4-benzoxazin-7-ylsulfonyl]-6-{2-oxo-7-azaspiro[3.5]nonan-7-yl}-4-[(3R,8S)-15-{[2-(trimethylsilyl)ethoxy]methyl}-2,5-dioxa-9,15,17-triazatetracyclo[8.7.0.0\hat{ }{3,8}.0\hat{ }{12,16}]heptadeca-1(10),11,13,16-tetraen-9-yl]pyridine-3-carboxamide as an orange solid (110 mg, 17.3%). LC-MS (ES, m/z) M+1: 963.

Synthesis of 4-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-5-fluoro-N-[(3R)-5-nitro-3-(oxan-4-yl)-3,4-dihydro-2H-1,4-benzoxazin-7-ylsulfonyl]-6-{2-oxo-7-azaspiro[3.5]nonan-7-yl}pyridine-3-carboxamide: Into an 8 mL vial, were added 5-fluoro-N-[(3R)-5-nitro-3-(oxan-4-yl)-3,4-dihydro-2H-1,4-benzoxazin-7-ylsulfonyl]-6-{2-oxo-7-azaspiro[3.5]nonan-7-yl}-4-[(3R,8S)-15-{[2-(trimethylsilyl)ethoxy]methyl}-2,5-dioxa-9,15,17-triazatetracyclo[8.7.0.0\hat{ }{3,8}.0\hat{ }{12,16}]heptadeca-1(10),11,13,16-tetraen-9-yl]pyridine-3-carboxamide (100 mg, 0.1 mmol, 1.0 eq), TBAF (543 mg, 2.0 mmol, 20.0 eq), ethane-1,2-diamine (125 mg, 2.0 mmol, 20.0 eq), THE (2 mL). The resulting mixture was stirred for 14 hours at 70° C. The resulting mixture was diluted with water (20 mL), and then extracted with CH₂Cl₂ (3×20 mL). The combined organic layers were washed with brine (20 mL), dried over anhydrous Na₂SO₄. After filtration, the filtrate was concentrated under vacuum. The crude residue was purified by Prep-TLC (CH₂Cl₂:MeOH=15:1) to give 4-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-5-fluoro-N-[(3R)-5-nitro-3-(oxan-4-yl)-3,4-dihydro-2H-1,4-benzoxazin-7-ylsulfonyl]-6-{2-oxo-7-azaspiro[3.5]nonan-7-yl}pyridine-3-carboxamide as a yellow solid (35 mg, 40.5%). LC-MS (ES, m/z) M+1: 833.

Synthesis of 6-{2-[(2R)-4-[(3,4-difluorophenyl)methyl]-2-(2-isopropoxyphenyl)piperazin-1-yl]-7-azaspiro[3.5]nonan-7-yl}-4-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-5-fluoro-N-[(3R)-5-nitro-3-(oxan-4-yl)-3,4-dihydro-2H-1,4-benzoxazin-7-ylsulfonyl]pyridine-3-carboxamide (Rotamer1): Into an 8 mL vial, were added 4-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-5-fluoro-N-[(3R)-5-nitro-3-(oxan-4-yl)-3,4-dihydro-2H-1,4-benzoxazin-7-ylsulfonyl]-6-{2-oxo-7-azaspiro[3.5]nonan-7-yl}pyridine-3-carboxamide (25 mg, 0.03 mmol, 1.0 eq) (3R)-1-[(3,4-difluorophenyl)methyl]-3-(2-isopropoxyphenyl)piperazine (10 mg, 0.03 mmol, 1.0 eq), ZnCl₂ (20 mg, 0.15 mmol, 5.0 eq), NaBH₃CN (9 mg, 0.15 mmol, 5.0 eq), MeOH (2 mL). The resulting mixture was stirred for 14 hours at 70° C. The reaction was quenched by the addition of water (5 mL), and extracted with CH₂Cl₂ (3×10 mL). The combined organic layers were washed with brine (10 mL), dried over anhydrous Na₂SO₄. After filtration, the filtrate was concentrated under vacuum. The crude product was purified by Flash-Prep-HPLC using the following conditions: Column: Xselect CSH C18 OBD Column 30*150 mm 5 μm, n; Mobile Phase A: water (0.1% NH₃—H₂O), Mobile Phase B: CH₃CN; Flow rate: 60 mL/min; Gradient: 15% B to 30% B in 9 min, 30% B; Wave Length: 220/254 nm; Finally, 6-{2-[(2R)-4-[(3,4-difluorophenyl)methyl]-2-(2-isopropoxyphenyl)piperazin-1-yl]-7-azaspiro[3.5]nonan-7-yl}-4-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-5-fluoro-N-[(3R)-5-nitro-3-(oxan-4-yl)-3,4-dihydro-2H-1,4-benzoxazin-7-ylsulfonyl]pyridine-3-carboxamide (Rotamer1) was obtained as a yellow solid (6.5 mg, 18.6%). LC-MS (ES, m/z) M+1: 1163. ¹H NMR (300 MHz, Chloroform-d) δ 8.85-8.61 (m, 1H), 8.56-8.21 (m, 3H), 7.60-7.40 (m, 2H), 7.28-7.14 (m, 2H), 7.14-6.95 (m, 3H), 6.94-6.79 (m, 2H), 6.75 (s, 1H), 6.16 (s, 1H), 4.77-3.81 (m, 10H), 3.79-2.98 (m, 13H), 2.97-2.54 (m, 4H), 2.48-2.10 (m, 2H), 1.62-1.20 (m, 20H).

### Example 229 Preparation of 6-{2-[(2R)-4-[(3,4-difluorophenyl)methyl]-2-(2-isopropoxyphenyl)piperazin-1-yl]-7-azaspiro[3.5]nonan-7-yl}-4-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-5-fluoro-N-[(3R)-5-nitro-3-(oxan-4-yl)-3,4-dihydro-2H-1,4-benzoxazin-7-ylsulfonyl]pyridine-3-carboxamide (rotamer 2)

Synthesis of 6-{2-[(2R)-4-[(3,4-difluorophenyl)methyl]-2-(2-isopropoxyphenyl)piperazin-1-yl]-7-azaspiro[3.5]nonan-7-yl}-4-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-5-fluoro-N-[(3R)-5-nitro-3-(oxan-4-yl)-3,4-dihydro-2H-1,4-benzoxazin-7-ylsulfonyl]pyridine-3-carboxamide (Rotamer2): Into an 8 mL vial, were added 4-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-5-fluoro-N-[(3R)-5-nitro-3-(oxan-4-yl)-3,4-dihydro-2H-1,4-benzoxazin-7-ylsulfonyl]-6-{2-oxo-7-azaspiro[3.5]nonan-7-yl}pyridine-3-carboxamide (25 mg, 0.03 mmol, 1.0 eq) (3R)-1-[(3,4-difluorophenyl)methyl]-3-(2-isopropoxyphenyl)piperazine (10 mg, 0.03 mmol, 1.0 eq), ZnCl₂ (20 mg, 0.15 mmol, 5.0 eq), NaBH₃CN (9 mg, 0.15 mmol, 5.0 eq), MeOH (2 mL). The resulting mixture was stirred for 14 hours at 70° C. The reaction was quenched by the addition of water (5 mL), and then extracted with CH₂Cl₂ (3×10 mL). The combined organic layers were washed with brine (10 mL), dried over anhydrous Na₂SO₄. After filtration, the filtrate was concentrated under vacuum. The crude product was purified by Flash-Prep-HPLC using the following conditions: Column: Xselect CSH C18 OBD Column 30*150 mm 5 μm, n; Mobile Phase A: water (0.1% NH₃—H₂O), Mobile Phase B: CH₃CN; Flow rate: 60 mL/min; Gradient: 15% B to 30% B in 9 min, 30% B; Wave Length: 220/254 nm; Finally, 6-{2-[(2R)-4-[(3,4-difluorophenyl)methyl]-2-(2-isopropoxyphenyl)piperazin-1-yl]-7-azaspiro[3.5]nonan-7-yl}-4-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-5-fluoro-N-[(3R)-5-nitro-3-(oxan-4-yl)-3,4-dihydro-2H-1,4-benzoxazin-7-ylsulfonyl]pyridine-3-carboxamide (Rotamer2) was obtained as a yellow solid (1.8 mg, 5.15%). LC-MS (ES, m/z) M+1: 1163. ¹H NMR (300 MHz, Chloroform-d) δ 8.85-8.61 (m, 1H), 8.56-8.21 (m, 3H), 7.60-7.40 (m, 2H), 7.28-6.79 (m, 10H), 6.30-6.10 (s, 1H), 4.70-4.30 (m, 3H), 4.20-3.30 (m, 20H), 3.29-2.40 (m, 4H), 1.62-1.20 (m, 21H).

### Example 230 Preparation of 4-(2-((R)-4-((R)-6,8-difluoroisochroman-4-yl)-2-(2-isopropoxyphenyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide (assumed)

Synthesis of (2-bromo-4,6-difluorophenyl)methanol: To a stirred solution of 2-bromo-4,6-difluorobenzoic acid (20.0 g, 67.5 mmol, 1.0 eq) in THF (160 mL) were added BH₃-Me₂S (51.2 g, 675.1 mmol, 10.0 eq) dropwise at 0° C. under N₂ atmosphere. The resulting mixture was stirred for 16 hours at 25° C. under N₂ atmosphere. The reaction was quenched by the addition of MeOH (200 mL) at 0° C. The resulting mixture was stirred for 1 hour at 80° C. The resulting mixture was concentrated under vacuum. The crude residue was applied onto a silica gel column and eluted with ethyl acetate/petroleum ether=1:1 to give (2-bromo-4,6-difluorophenyl)methanol as a white solid (16.0 g, 73.0%). ¹H NMR (300 MHz, Chloroform-d) δ 7.19 (ddd, J=7.9, 2.5, 1.7 Hz, 1H), 6.86 (ddd, J=9.6, 8.6, 2.5 Hz, 1H), 4.82 (d, J=2.2 Hz, 2H).

Synthesis of 2-((allyloxy)methyl)-1-bromo-3,5-difluorobenzene: Into a 500 mL 3-necked round-bottom flask, a solution of (2-bromo-4,6-difluorophenyl)methanol (16.0 g, 63.3 mmol, 1.0 eq) in THE (200 mL) was treated with NaH (3.0 g, 126.7 mmol, 2.0 eq) for 30 min at 0° C. under nitrogen atmosphere followed by the addition of allyl bromide (11.5 g, 95.0 mmol, 1.5 eq) dropwise at 0° C. The resulting mixture was stirred for 16 hours at 25° C. under N₂ atmosphere. The mixture was allowed to cool down to 0° C. The reaction was quenched by the addition of NH₄Cl (aq.) (100 mL), extracted with ethyl acetate (3×50 mL). The combined organic layers were washed with brine (100 mL), dried over anhydrous Na₂SO₄. After filtration, the filtrate was concentrated under vacuum. The crude residue was applied onto a silica gel column and eluted with ethyl acetate/petroleum ether=1:2 to give 2-((allyloxy)methyl)-1-bromo-3,5-difluorobenzene as a white solid (14.0 g, 75.9%). ¹H NMR (300 MHz, Chloroform-d) δ 7.20 (ddd, J=8.0, 2.5, 1.7 Hz, 1H), 6.84 (ddd, J=9.5, 8.6, 2.5 Hz, 1H), 5.97 (ddt, J=17.2, 10.4, 5.8 Hz, 1H), 5.42-5.18 (m, 2H), 4.63 (d, J=2.4 Hz, 2H), 4.08 (dt, J=5.8, 1.4 Hz, 2H).

Synthesis of 6,8-difluoro-4-methyleneisochromane: Into a 500 mL 3-necked round-bottom flask were added 2-((allyloxy)methyl)-1-bromo-3,5-difluorobenzene (14.0 g, 51.2 mmol, 1.0 eq), Pd(OAc)₂ (2.3 g, 10.2 mmol, 0.2 eq), PPh₃ (2.7 g, 10.2 mmol, 0.2 eq), DIEA (9.9 g, 76.9 mmol, 1.5 eq) and MeCN (150 mL) at 25° C. The resulting mixture was stirred for 16 hours at 80° C. under N₂ atmosphere. The resulting mixture was filtered, the filter cake was washed with ethyl acetate (3×50 mL). After filtration, the filtrate was concentrated under vacuum. The crude residue was applied onto a silica gel column and eluted with ethyl acetate/petroleum ether=1:3 to give 6,8-difluoro-4-methyleneisochromane as a white solid (1.4 g, 13.9%). ¹H NMR (400 MHz, Chloroform-d) δ 7.26-7.17 (m, 1H), 6.74 (td, J=9.1, 2.4 Hz, 1H), 5.64 (s, 1H), 5.16 (s, 1H), 4.83 (s, 2H), 4.42 (d, J=1.3 Hz, 2H).

Synthesis of 26,8-difluoroisochroman-4-one: Into a 100 mL bottom flask were added 6,8-difluoro-4-methyleneisochromane (1.4 g, 6.6 mmol, 1.0 eq), NalO₄ (4.2 g, 19.8 mmol, 3.0 eq), K₂OsO₄.2H₂O (1.7 g, 4.6 mmol, 0.7 eq), t-BuOH (6 mL), THE (6 mL), and H₂O (1 mL) at 0° C. The resulting mixture was stirred for 16 hours at 25° C. under N₂ atmosphere. The reaction was quenched by the addition of Na₂SO₃ (aq.) (100 mL) at 0° C. The resulting mixture was extracted with ethyl acetate (3×50 mL). The combined organic layers were washed with brine (50 mL), dried over anhydrous Na₂SO₄. After filtration, the filtrate was concentrated under vacuum. The crude residue was applied onto a silica gel column and eluted with ethyl acetate/petroleum ether=1:3 to give 6,8-difluoroisochroman-4-one as a yellow solid (800 mg, 52.5%). ¹H NMR (300 MHz, Chloroform-d) δ 7.58 (ddd, J=8.2, 2.6, 1.2 Hz, 1H), 7.09 (ddd, J=9.2, 8.3, 2.5 Hz, 1H), 4.95 (s, 2H), 4.42-4.36 (m, 2H).

Synthesis of 6,8-difluoroisochroman-4-ol: Into a 40 mL bottom flask were added 6,8-difluoroisochroman-4-one (800 mg, 3.2 mmol, 1.0 eq) and MeOH (10 mL) at 0° C. The resulting mixture was stirred for 16 hours at 25° C. The resulting mixture was concentrated under vacuum and quenched by the addition of H₂O (10 mL) and then extracted with ethyl acetate (3×15 mL). The combined organic layers were washed with brine (10 mL), dried over anhydrous Na₂SO₄. After filtration, the filtrate was concentrated under vacuum. The crude residue was applied onto a silica gel column and eluted with ethyl acetate/petroleum ether=1:1 to give 6,8-difluoroisochroman-4-ol as a yellow solid (600 mg, 84.9%). ¹H NMR (300 MHz, Chloroform-d) δ 7.02 (dt, J=8.7, 1.9 Hz, 1H), 6.76 (td, J=9.2, 2.5 Hz, 1H), 4.88-4.79 (m, 1H), 4.64-4.45 (m, 2H), 3.95 (ddd, J=45.9, 12.0, 3.2 Hz, 2H).

Synthesis of 4-chloro-6,8-difluoroisochromane: Into a 40 mL bottom flask, were placed 6,8-difluoroisochroman-4-ol (600 mg, 2.7 mmol, 1.0 eq) in CHCl₃ (1 mL) were added SOCl₂ (662 mg, 5.5 mmol, 2.0 eq) dropwise at 0° C. under N₂ atmosphere. The resulting mixture was stirred for 16 hours at 25° C. under N₂ atmosphere. The reaction was quenched by the addition of H₂O (10 mL), and then extracted with ethyl acetate (3×5 mL). The combined organic layers were washed with brine (5 mL), dried over anhydrous Na₂SO₄. After filtration, the filtrate was concentrated under vacuum. The crude residue was applied onto a silica gel column and eluted with ethyl acetate/petroleum ether=1:3 to give 4-chloro-6,8-difluoroisochromane as a yellow solid (500 mg, 76.7%). ¹H NMR (300 MHz, Chloroform-d) δ 7.60-7.53 (m, 1H), 7.09 (td, J=8.8, 2.5 Hz, 1H), 4.95 (s, 2H), 4.39 (s, 2H), 4.33 (t, J=6.7 Hz, 1H).

Synthesis of (S)-2-chloro-5-((R)-3-(2-isopropoxyphenyl)piperazin-1-yl)-3-methoxy-5,8-dihydro-6H-pyrano[3,4-b]pyridine (assumed) and (R)-2-chloro-5-((R)-3-(2-isopropoxyphenyl)piperazin-1-yl)-3-methoxy-5,8-dihydro-6H-pyrano[3,4-b]pyridine (assumed): Into an 8 mL bottom flask were added 4-chloro-6,8-difluoroisochromane (500 mg, 1.2 mmol, 1.0 eq), (R)-2-(2-isopropoxyphenyl)piperazine (280 mg, 1.2 mmol, 1.0 eq), DIEA (496 mg, 3.8 mmol, 3.0 eq) and MeCN (6 mL) at 25° C. The resulting mixture was stirred for 16 hours at 80° C. The resulting mixture was diluted with ethyl acetate (30 mL) and then washed with H₂O (5 mL). The combined organic layers were washed with brine (5 mL), dried over anhydrous Na₂SO₄. After filtration, the filtrate was concentrated under vacuum. The crude residue was applied onto a silica gel column and eluted with ethyl acetate/petroleum ether=10:1 to give (R)-1-((R)-6,8-difluoroisochroman-4-yl)-3-(2-isopropoxyphenyl)piperazine (assumed) as a yellow oil (80 mg, 14.9%) and (R)-1-((S)-6,8-difluoroisochroman-4-yl)-3-(2-isopropoxyphenyl)piperazine (assumed) as a yellow oil (70 mg, 11.8%). 6A-¹H NMR (400 MHz, Chloroform-d) δ 7.52 (dd, J=7.6, 1.7 Hz, 1H), 7.28-7.16 (m, 2H), 6.97-6.86 (m, 2H), 6.72 (td, J=9.1, 2.5 Hz, 1H), 4.80 (d, J=15.2 Hz, 1H), 4.68-4.57 (m, 2H), 4.42-4.35 (m, 1H), 4.18 (dd, J=12.0, 4.7 Hz, 1H), 3.85 (dd, J=12.0, 4.5 Hz, 1H), 3.79-3.67 (m, 2H), 3.11 (t, J=11.4 Hz, 2H), 2.94 (td, J=10.9, 3.6 Hz, 1H), 2.76 (t, J=10.6 Hz, 1H), 2.69 (d, J=15.4 Hz, 2H), 1.41 (t, J=5.8 Hz, 6H). 6B-¹H NMR (400 MHz, Chloroform-d) δ 7.49 (dd, J=7.6, 1.7 Hz, 1H), 7.29 (d, J=10.0 Hz, 1H), 7.18 (ddd, J=9.1, 7.4, 1.7 Hz, 1H), 6.88 (td, J=7.5, 1.1 Hz, 1H), 6.80 (d, J=8.3 Hz, 1H), 6.68 (td, J=9.2, 2.5 Hz, 1H), 4.71 (d, J=15.3 Hz, 1H), 4.60 (d, J=15.2 Hz, 1H), 4.49 (p, J=6.0 Hz, 1H), 4.19-4.07 (m, 2H), 3.90 (dd, J=11.8, 4.7 Hz, 1H), 3.82-3.71 (m, 1H), 3.30-3.23 (m, 1H), 3.18 (td, J=11.2, 10.7, 4.0 Hz, 1H), 3.02-2.88 (m, 3H), 2.78 (dt, J=10.6, 2.2 Hz, 1H), 2.34 (t, J=10.4 Hz, 1H), 1.36-1.23 (m, 4H), 1.10 (d, J=6.0 Hz, 2H).

Synthesis of 4-(2-((R)-4-((R)-6,8-difluoroisochroman-4-yl)-2-(2-isopropoxyphenyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide (assumed): Into an 8 mL vial were added (R)-1-((R)-6,8-difluoroisochroman-4-yl)-3-(2-isopropoxyphenyl)piperazine (assumed) (20 mg, 0.1 mmol, 1.0 eq), 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-N—[(3R)-5-nitro-3-(oxan-4-yl)-3,4-dihydro-2H-1,4-benzoxazin-7-ylsulfonyl]-4-{2-oxo-7-azaspiro[3.5]nonan-7-yl}benzamide (27 mg, 0.1 mmol, 1.0 eq), NaBH₃CN (10 mg, 0.2 mmol, 5.0 eq), ZnCl₂ (23 mg, 0.1 mmol, 5.0 eq) and MeOH (1 mL). The resulting mixture was stirred for 16 hours at 50° C. The reaction was quenched by the addition of water (5 mL), and then extracted with CH₂Cl₂ (3×5 mL). The combined organic layers were washed with brine (5 mL), dried over anhydrous Na₂SO₄. After filtration, the filtrate was concentrated under vacuum. The crude residue was purified by Prep-TLC (CH₂Cl₂/MeOH=15:1) to give 4-(2-((R)-4-((R)-6,8-difluoroisochroman-4-yl)-2-(2-isopropoxyphenyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide (assumed) as a yellow solid (13 mg, 31.1%). LC-MS (ES, m/z) M+1: 1187. ¹H NMR (300 MHz, Chloroform-d) δ 12.64 (s, 1H), 8.46 (s, 1H), 8.38 (s, 1H), 8.31 (d, J=2.2 Hz, 2H), 8.21 (s, 1H), 8.13 (d, J=9.2 Hz, 2H), 7.35 (s, 2H), 7.19 (s, 1H), 6.69 (s, 3H), 6.43 (s, 2H), 4.78-4.75 (m, 4H), 4.69-4.64 (m, 2H), 4.66-4.46 (m, 5H), 4.44-4.42 (m, 1H), 4.21-4.19 (m, 2H), 4.16-4.02 (m, 7H), 3.95-3.91 (m, 3H), 3.87-3.85 (m, 2H), 3.76-3.74 (m, 1H), 3.70-3.68 (m, 2H), 3.51-3.49 (m, 2H), 3.39-3.36 (m, 4H), 2.89-2.87 (m, 2H), 2.74-2.72 (m, 2H), 2.63-2.60 (m, 4H), 2.13-2.11 (m, 2H), 2.05-2.01 (m, 1H), 1.81-1.79 (m, 3H), 1.28-1.26 (m, 1H), 1.20-1.17 (m, 2H), 1.07-1.05 (m, 1H).

### Example 231 Preparation of 4-(2-((R)-4-((S)-6,8-difluoroisochroman-4-yl)-2-(2-isopropoxyphenyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide (assumed)

Synthesis of 4-(2-((R)-4-((S)-6,8-difluoroisochroman-4-yl)-2-(2-isopropoxyphenyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide (assumed): Into an 8 mL vial were added (R)-1-((S)-6,8-difluoroisochroman-4-yl)-3-(2-isopropoxyphenyl)piperazine (assumed) (20 mg, 0.1 mmol, 1.0 eq), 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-N-[(3R)-5-nitro-3-(oxan-4-yl)-3,4-dihydro-2H-1,4-benzoxazin-7-ylsulfonyl]-4-{2-oxo-7-azaspiro[3.5]nonan-7-yl}benzamide (27 mg, 0.1 mmol, 1.0 eq), NaBH₃CN (10 mg, 0.2 mmol, 5.0 eq), ZnCl₂ (23 mg, 0.1 mmol, 5.0 eq) and MeOH (1 mL). The resulting mixture was stirred for 16 hours at 50° C. The reaction was quenched by the addition of water (5 mL), and then extracted with CH₂Cl₂ (3×5 mL). The combined organic layers were washed with brine (5 mL), dried over anhydrous Na₂SO₄. After filtration, the filtrate was concentrated under vacuum. The crude residue was purified by Prep-TLC (CH₂Cl₂/MeOH=15:1) to give 4-(2-((R)-4-((S)-6,8-difluoroisochroman-4-yl)-2-(2-isopropoxyphenyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide (assumed) as a yellow solid (11 mg, 27.1%). LC-MS (ES, m/z) M+1: 1187. ¹H NMR (300 MHz, Chloroform-d) δ 12.65 (s, 1H), 8.48 (d, J=13.4 Hz, 1H), 8.40-8.28 (m, 2H), 8.13 (d, J=9.2 Hz, 1H), 7.46 (d, J=2.1 Hz, 1H), 7.38 (d, J=7.8 Hz, 3H), 7.27-7.07 (m, 2H), 6.82-6.80 (m, 2H), 6.67 (d, J=17.1 Hz, 1H), 6.44 (s, 1H), 6.14 (s, 1H), 4.73-4.55 (m, 2H), 4.53-4.51 (m, 1H), 4.44-4.43 (m, 1H), 4.16-3.82 (m, 6H), 3.81-3.68 (m, 6H), 3.51-3.49 (m, 1H), 3.44-3.43 (m, 3H), 3.39-3.36 (m, 2H), 3.08-3.05 (m, 4H), 2.99-2.96 (m, 6H), 2.64-2.62 (m, 8H), 2.41-2.40 (m, 3H), 1.79-1.76 (m, 3H), 1.70-1.69 (m, 1H), 1.34-1.31 (m, 3H), 1.15-1.13 (m, 3H).

### Example 232 Preparation of 4-(2-((R)-2-(2-isopropoxyphenyl)-4-((S)-3-methoxy-2-((S)-3-methylmorpholino)-5,8-dihydro-6H-pyrano[3,4-b]pyridin-5-yl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide (assumed)

Synthesis of 5-bromo-2-chloro-6-(hydroxymethyl)pyridin-3-ol: A solution of 5-bromo-2-chloropyridin-3-ol (20.0 g, 95.9 mmol, 1.0 eq) in H₂O (100 mL) was treated with NaHCO₃ (24.1 g, 287.8 mmol, 3.0 eq) for 30 min at 25° C. under nitrogen atmosphere, which was followed by the addition of 30% formalin (100 mL) dropwise at 90° C. The resulting mixture was stirred for 16 hours at 90° C. under N₂ atmosphere. The mixture was allowed to cool down to 0° C. The mixture was acidified to pH=1 with 1 M HCl. The precipitated solids were collected by filtration and washed with H₂O (3×50 mL). The filtrate extracted with ethyl acetate (3×100 mL). The combined organic layers were washed with brine (100 mL), dried over anhydrous Na₂SO₄. After filtration, the filtrate was concentrated under vacuum. The combined filtration and filtrate, to give 5-bromo-2-chloro-6-(hydroxymethyl)pyridin-3-ol as a red solid (18.0 g, crude).

Synthesis of (3-bromo-6-chloro-5-methoxypyridin-2-yl)methanol: Into a 500 MI 3-necked round-bottom flask, were added 5-bromo-2-chloro-6-(hydroxymethyl)pyridin-3-ol (18.0 g, 75.4 mmol, 1.0 eq), methyl iodide (21.4 g, 150.9 mmol, 2.0 eq), K₂CO₃ (31.3 g, 226.4 mmol, 3.0 eq) and DMF (200 mL) at 25° C. The resulting mixture was stirred for 16 hours at 90° C. The resulting mixture was filtered, the filter cake was washed with ethyl acetate (3×150 mL). The filtrate was washed with H₂O (3×100 mL). The combined organic layers were washed with brine (100 mL), dried over anhydrous Na₂SO₄. After filtration, the filtrate was concentrated under vacuum. The crude residue was applied onto a silica gel column and eluted with ethyl acetate/petroleum ether=1:2 to give (3-bromo-6-chloro-5-methoxypyridin-2-yl)methanol as a colorless oil (16.0 g, 83.9%). ¹H NMR (300 MHz, DMSO-d₆) δ 7.84 (s, 1H), 5.31 (t, J=6.0 Hz, 1H), 4.52 (d, J=6.0 Hz, 2H), 3.93 (s, 3H).

Synthesis of 2-((allyloxy)methyl)-3-bromo-6-chloro-5-methoxypyridine: Into a 500 mL 3-necked round-bottom flask, a solution of (3-bromo-6-chloro-5-methoxypyridin-2-yl)methanol (16.0 g, 63.3 mmol, 1.0 eq) in THE (200 mL) was treated with NaH (3.0 g, 126.7 mmol, 2.0 eq) for 30 min at 0° C. under nitrogen atmosphere followed by the addition of allyl bromide (11.5 g, 95.0 mmol, 1.5 eq) dropwise at 0° C. The resulting mixture was stirred for 16 hours at 25° C. under N₂ atmosphere. The mixture was allowed to cool down to 0° C. The reaction was quenched by the addition of NH₄Cl (aq) (100 mL) and then extracted with ethyl acetate (3×50 mL). The combined organic layers were washed with brine (100 mL), dried over anhydrous Na₂SO₄. After filtration, the filtrate was concentrated under vacuum. The crude residue was applied onto a silica gel column and eluted with ethyl acetate/petroleum ether=1:2 to give 2-((allyloxy)methyl)-3-bromo-6-chloro-5-methoxypyridine as a white solid (15.0 g, 80.9%). ¹H NMR (300 MHz, DMSO-d₆) δ 7.88 (s, 1H), 5.91 (ddt, J=17.3, 10.6, 5.4 Hz, 1H), 5.28 (dq, J=17.3, 1.7 Hz, 1H), 5.22-5.11 (m, 1H), 4.53 (s, 2H), 4.03 (dt, J=5.4, 1.5 Hz, 2H), 3.94 (s, 3H).

Synthesis of 2-chloro-3-methoxy-5-methylene-5,8-dihydro-6H-pyrano[3,4-b]pyridine: Into a 500 mL 3-necked round-bottom flask were added 2-((allyloxy)methyl)-3-bromo-6-chloro-5-methoxypyridine (15.0 g, 51.2 mmol, 1.0 eq), Pd(OAc)₂ (2.3 g, 10.2 mmol, 0.2 eq), PPh₃ (2.7 g, 10.2 mmol, 0.2 eq), DIEA (9.9 g, 76.9 mmol, 1.5 eq) and MeCN (150 mL) at 25° C. The resulting mixture was stirred for 16 hours at 80° C. under N₂ atmosphere. The resulting mixture was filtered, the filter cake was washed with ethyl acetate (3×50 mL). The filtrate was concentrated under vacuum. The crude residue was applied onto a silica gel column and eluted with ethyl acetate/petroleum ether=1:3 to give 2-chloro-3-methoxy-5-methylene-5,8-dihydro-6H-pyrano[3,4-b]pyridine as a white solid (1.4 g, 12.9%). ¹H NMR (300 MHz, Chloroform-d) δ 7.41 (s, 1H), 5.63 (d, J=1.1 Hz, 1H), 5.21 (t, J=1.4 Hz, 1H), 4.80 (s, 2H), 4.44 (t, J=1.2 Hz, 2H), 3.98 (s, 3H).

Synthesis of 2-chloro-3-methoxy-6H-pyrano[3,4-b]pyridin-5(8H)-one: Into a 100 mL bottom flask were added 2-chloro-3-methoxy-5-methylene-5,8-dihydro-6H-pyrano[3,4-b]pyridine (1.4 g, 6.6 mmol, 1.0 eq), NalO₄ (4.2 g, 19.8 mmol, 3.0 eq), K₂OsO₄.2H₂O (1.7 g, 4.6 mmol, 0.7 eq), t-BuOH (6 mL), THE (6 mL), and H₂O (1 mL) at 0° C. The resulting mixture was stirred for 16 hours at 25° C. under N₂ atmosphere. The reaction was quenched by the addition of Na₂SO₃ (aq.) (100 mL), and then extracted with ethyl acetate (3×50 mL). The combined organic layers were washed with brine (50 mL), dried over anhydrous Na₂SO₄. After filtration, the filtrate was concentrated under vacuum. The crude residue was applied onto a silica gel column and eluted with ethyl acetate/petroleum ether=1:3 to give 2-chloro-3-methoxy-6H-pyrano[3,4-b]pyridin-5(8H)-one as a yellow solid (700 mg, 49.5%). ¹H NMR (300 MHz, Chloroform-d) δ 7.73 (d, J=5.3 Hz, 1H), 4.91 (s, 2H), 4.39 (d, J=0.7 Hz, 2H), 4.01 (s, 3H).

Synthesis of 2-chloro-3-methoxy-5,8-dihydro-6H-pyrano[3,4-b]pyridin-5-ol: Into a 40 mL bottom flask were added 2-chloro-3-methoxy-6H-pyrano[3,4-b]pyridin-5(8H)-one (700 mg, 3.2 mmol, 1.0 eq) and MeOH (10 mL) at 0° C. The resulting mixture was stirred for 16 hours at 25° C. The resulting mixture was concentrated under vacuum and quenched by the addition of H₂O (10 mL) and then extracted with ethyl acetate (3×15 mL). The combined organic layers were washed with brine (10 mL), dried over anhydrous Na₂SO₄. After filtration, the filtrate was concentrated under vacuum. The crude residue was applied onto a silica gel column and eluted with ethyl acetate/petroleum ether=1:1 to give 2-chloro-3-methoxy-5,8-dihydro-6H-pyrano[3,4-b]pyridin-5-ol as a yellow solid (600 mg, 84.9%). ¹H NMR (300 MHz, Chloroform-d) δ 7.33 (s, 1H), 4.80 (d, J=15.9 Hz, 1H), 4.66 (d, J=9.9 Hz, 1H), 4.06 (dd, J=12.1, 3.4 Hz, 1H), 3.96 (s, 3H), 3.92 (dd, J=12.1, 3.0 Hz, 1H).

Synthesis of 2,5-dichloro-3-methoxy-5,8-dihydro-6H-pyrano[3,4-b]pyridine: Into a 40 mL bottom flask, to a stirred solution of 2-chloro-3-methoxy-5,8-dihydro-6H-pyrano[3,4-b]pyridin-5-ol (600 mg, 2.7 mmol, 1.0 eq) in CHCl₃ (1 mL) were added SOCl₂ (662 mg, 5.5 mmol, 2.0 eq) dropwise at 0° C. under N₂ atmosphere. The resulting mixture was stirred for 16 hours at 25° C. under N₂ atmosphere. The reaction was quenched by the addition of H₂O (10 mL) and extracted with ethyl acetate (3×5 mL). The combined organic layers were washed with brine (5 mL), dried over anhydrous Na₂SO₄. After filtration, the filtrate was concentrated under vacuum. The crude residue was applied onto a silica gel column and eluted with ethyl acetate/petroleum ether=1:3 to give 2,5-dichloro-3-methoxy-5,8-dihydro-6H-pyrano[3,4-b]pyridine as a yellow solid. (500 mg, 76.7%). ¹H NMR (300 MHz, Chloroform-d) δ 7.32 (s, 1H), 5.11 (t, J=5.2 Hz, 1H), 4.84 (d, J=15.9 Hz, 1H), 4.73 (d, J=15.9 Hz, 1H), 4.24 (dd, J=12.2, 4.4 Hz, 1H), 4.06-3.98 (m, 1H), 3.97 (s, 3H).

Synthesis of (S)-2-chloro-5-((R)-3-(2-isopropoxyphenyl)piperazin-1-yl)-3-methoxy-5,8-dihydro-6H-pyrano[3,4-b]pyridine (assumed) and (R)-2-chloro-5-((R)-3-(2-isopropoxyphenyl)piperazin-1-yl)-3-methoxy-5,8-dihydro-6H-pyrano[3,4-b]pyridine (assumed): Into an 8 mL bottom flask were added 2,5-dichloro-3-methoxy-5,8-dihydro-6H-pyrano[3,4-b]pyridine [3,4-b]pyridine (150 mg, 0.6 mmol, 1.0 eq), (R)-2-(2-isopropoxyphenyl)piperazine (141 mg, 0.6 mmol, 1.0 eq), DIEA (248 mg, 1.9 mmol, 3.0 eq) and MeCN (3 mL) at 25° C. The resulting mixture was stirred for 16 hours at 80° C. The resulting mixture was diluted with ethyl acetate (30 mL). The resulting mixture was washed H₂O (5 mL). The combined organic layers were washed with brine (5 mL), dried over anhydrous Na₂SO₄. After filtration, the filtrate was concentrated under vacuum. The crude residue was applied onto a silica gel column and eluted with ethyl acetate/petroleum ether=10:1 to give (S)-2-chloro-5-((R)-3-(2-isopropoxyphenyl)piperazin-1-yl)-3-methoxy-5,8-dihydro-6H-pyrano[3,4-b]pyridine (assumed) as a yellow oil (40 mg, 14.9%) and (R)-2-chloro-5-((R)-3-(2-isopropoxyphenyl)piperazin-1-yl)-3-methoxy-5,8-dihydro-6H-pyrano[3,4-b]pyridine (assumed) as a yellow oil (35 mg, 11.8%).

Synthesis of (S)-5-((R)-3-(2-isopropoxyphenyl)piperazin-1-yl)-3-methoxy-2-((S)-3-methylmorpholino)-5,8-dihydro-6H-pyrano[3,4-b]pyridine (assumed): Into an 8 mL bottom flask were added (3R)-1-{2-chloro-3-methoxy-5H,6H,8H-pyrano[3,4-b]pyridin-5-yl}-3-(2-isopropoxyphenyl)piperazine (30 mg, 0.1 mmol, 1.0 eq), (S)-3-methylmorpholine (14 mg, 0.2 mmol, 2.0 eq), Pd(PPh₃)₂Cl₂ (5 mg, 0.01 mmol, 0.1 eq), t-BuOK (16 mg, 0.2 mmol, 2.0 eq) and 1,4-dioxane (1 mL) at 25° C. The resulting mixture was stirred for 16 hours at 100° C. under N₂ atmosphere. The resulting mixture was filtered, the filter cake was washed with EA (3×5 mL). After filtration, the filtrate was concentrated under vacuum. The crude residue was applied onto a silica gel column and eluted with ethyl acetate/petroleum ether=10:1 to give (S)-5-((R)-3-(2-isopropoxyphenyl)piperazin-1-yl)-3-methoxy-2-((S)-3-methylmorpholino)-5,8-dihydro-6H-pyrano[3,4-b]pyridine (assumed) as a yellow oil (20 mg, 57.7%). LC-MS (ES, m/z) M+1: 483.

Synthesis of 4-(2-((R)-2-(2-isopropoxyphenyl)-4-((S)-3-methoxy-2-((S)-3-methylmorpholino)-5,8-dihydro-6H-pyrano[3,4-b]pyridin-5-yl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide (assumed): Into an 8 mL vial were added (S)-5-((R)-3-(2-isopropoxyphenyl)piperazin-1-yl)-3-methoxy-2-((S)-3-methylmorpholino)-5,8-dihydro-6H-pyrano[3,4-b]pyridine (assumed) (12 mg, 0.1 mmol, 1.0 eq), 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-N-[(3R)-5-nitro-3-(oxan-4-yl)-3,4-dihydro-2H-1,4-benzoxazin-7-ylsulfonyl]-4-{2-oxo-7-azaspiro[3.5]nonan-7-yl}benzamide (27 mg, 0.1 mmol, 1.0 eq), NaBH₃CN (10 mg, 0.2 mmol, 5.0 eq), ZnCl₂ (23 mg, 0.1 mmol, 5.0 eq) and MeOH (1 mL). The resulting mixture was stirred for overnight at 50° C. The reaction was quenched by the addition of water (5 mL) and extracted with CH₂Cl₂ (3×5 mL). The combined organic layers were washed with brine (5 mL), dried over anhydrous Na₂SO₄. After filtration, the filtrate was concentrated under vacuum. The crude residue was purified by Prep-TLC (CH₂Cl₂/MeOH=15:1) to give 4-(2-((R)-2-(2-isopropoxyphenyl)-4-((S)-3-methoxy-2-((S)-3-methylmorpholino)-5,8-dihydro-6H-pyrano[3,4-b]pyridin-5-yl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide (assumed) as a yellow solid (5 mg, 12.1%). LC-MS (ES, m/z) M+1: 1281. ¹H NMR (300 MHz, Methanol-d₄) δ 8.03 (s, 1H), 7.47 (d, J=7.3 Hz, 3H), 7.30 (s, 3H), 6.99 (s, 3H), 6.66 (s, 1H), 6.56 (d, J=16.4 Hz, 1H), 5.99 (d, J=3.4 Hz, 1H), 4.61-4.59 (m, 1H), 4.42-4.38 (m, 2H), 4.19-4.17 (m, 6H), 4.01-3.98 (m, 6H), 3.88-3.86 (m, 3H), 3.70-3.66 (m, 3H), 3.60-3.56 (m, 6H), 3.45-3.41 (m, 6H), 2.83-2.81 (m, 3H), 1.73-1.70 (m, 4H), 1.63-1.61 (m, 6H), 1.49-1.47 (m, 8H), 1.38-1.35 (m, 6H), 1.31-1.30 (m, 2H), 1.09-1.06 (m, 3H).

### Example 233 Preparation of 4-(2-((R)-2-(2-isopropoxyphenyl)-4-((R)-3-methoxy-2-((S)-3-methylmorpholino)-5,8-dihydro-6H-pyrano[3,4-b]pyridin-5-yl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide (assumed)

Synthesis of (R)-5-((R)-3-(2-isopropoxyphenyl)piperazin-1-yl)-3-methoxy-2-((S)-3-methylmorpholino)-5,8-dihydro-6H-pyrano[3,4-b]pyridine (assumed): Into an 8 mL vial were added (R)-2-chloro-5-((R)-3-(2-isopropoxyphenyl)piperazin-1-yl)-3-methoxy-5,8-dihydro-6H-pyrano[3,4-b]pyridine (assumed) (30 mg, 0.1 mmol, 1.0 eq), (S)-3-methylmorpholine (14 mg, 0.2 mmol, 2.0 eq), Pd(PPh₃)₂Cl₂ (5 mg, 0.01 mmol, 0.1 eq), t-BuOK (24 mg, 0.2 mmol, 2.0 eq) and 1,4-dioxane (3 mL) at 25° C. The resulting mixture was stirred for 16 hours at 100° C. under N₂ atmosphere. The resulting mixture was filtered, the filter cake was washed with EA (3×5 mL). The filtrate was concentrated under vacuum. After filtration, the filtrate was concentrated under vacuum. The crude residue was applied onto a silica gel column and eluted with ethyl acetate/petroleum ether=10:1 to give (R)-5-((R)-3-(2-isopropoxyphenyl)piperazin-1-yl)-3-methoxy-2-((S)-3-methylmorpholino)-5,8-dihydro-6H-pyrano[3,4-b]pyridine (assumed) as a yellow oil (15 mg, 43.3%). LC-MS (ES, m/z) M+1: 483.

Synthesis of 4-(2-((R)-2-(2-isopropoxyphenyl)-4-((R)-3-methoxy-2-((S)-3-methylmorpholino)-5,8-dihydro-6H-pyrano[3,4-b]pyridin-5-yl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide (assumed): Into an 8 mL vial were added (R)-5-((R)-3-(2-isopropoxyphenyl)piperazin-1-yl)-3-methoxy-2-((S)-3-methylmorpholino)-5,8-dihydro-6H-pyrano[3,4-b]pyridine (assumed) (12 mg, 0.1 mmol, 1.0 eq), 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-N-[(3R)-5-nitro-3-(oxan-4-yl)-3,4-dihydro-2H-1,4-benzoxazin-7-ylsulfonyl]-4-{2-oxo-7-azaspiro[3.5]nonan-7-yl}benzamide (27 mg, 0.1 mmol, 1.0 eq), NaBH₃CN (10 mg, 0.2 mmol, 5.0 eq), ZnCl₂ (23 mg, 0.1 mmol, 5.0 eq) and MeOH (1 mL). The resulting mixture was stirred for 16 hours at 50° C. The reaction was quenched by the addition of water (5 mL) and then extracted with CH₂Cl₂ (3×5 mL). The combined organic layers were washed with brine (5 mL), dried over anhydrous Na₂SO₄. After filtration, the filtrate was concentrated under vacuum. The crude residue was purified by Prep-TLC (CH₂Cl₂/MeOH=15:1) to give 4-(2-((R)-2-(2-isopropoxyphenyl)-4-((R)-3-methoxy-2-((S)-3-methylmorpholino)-5,8-dihydro-6H-pyrano[3,4-b]pyridin-5-yl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide (assumed) as a yellow solid (3 mg, 9.1%). LC-MS (ES, m/z) M+1: 1281. ¹H NMR (300 MHz, Methanol-d₄) δ 7.37 (s, 1H), 7.30-7.25 (m, 2H), 6.96-6.84 (m, 2H), 6.81-6.74 (m, 4H), 6.67-6.52 (m, 4H), 4.50-4.40 (m, 4H), 4.47-4.32 (m, 6H), 4.18-4.15 (m, 6H), 4.01-3.94 (m, 3H), 3.86-3.84 (m, 7H), 3.69-3.66 (m, 5H), 3.44-3.40 (m, 4H), 2.45-2.41 (m, 6H), 1.74-1.71 (m, 3H), 1.68-1.63 (m, 3H) 1.48-1.45 (m, 3H), 1.31-1.29 (m, 5H), 1.27-1.25 (m, 7H), 1.08 (m, 3H).

### Example 234 Preparation of 6-{2-[(2R)-4-[(3,4-difluorophenyl)methyl]-2-(2-isopropoxyphenyl)piperazin-1-yl]-7-azaspiro[3.5]nonan-7-yl}-2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-5-fluoro-N-[(3R)-5-nitro-3-(oxan-4-yl)-3,4-dihydro-2H-1,4-benzoxazin-7-ylsulfonyl]pyridine-3-carboxamide (assumed)

Synthesis of 6-bromo-2-iodo-5-methylpyridin-3-amine: Into a 500 mL round-bottom flask were added 6-bromo-5-methylpyridin-3-amine (20.0 g, 106.9 mmol, 1.0 eq), NIS (36.1 g, 160.4 mmol, 1.5 eq) and DMF (150 mL). The resulting mixture was stirred for overnight at 25° C. The resulting mixture was diluted with EtOAc (200 mL). The resulting mixture was washed with 50 mL of anhydrous sodium sulfite and extracted with EtOAc (3×50 mL). The combined organic layers were washed with brine (50 mL), dried over anhydrous Na₂SO₄. After filtration, the filtrate was concentrated under vacuum. The crude residue was applied onto a silica gel column and eluted with ethyl acetate/petroleum ether=1:1 to give 6-bromo-2-iodo-5-methylpyridin-3-amine as a white solid (30.0 g, 89.7%). ¹H NMR (400 MHz, Chloroform-d) δ 6.82 (s, 1H), 3.97 (s, 2H), 2.27 (s, 3H).

Synthesis of 2-bromo-5-fluoro-6-iodo-3-methylpyridine: A solution of 6-bromo-2-iodo-5-methylpyridin-3-amine (25.0 g, 80.0. mmol, 1.0 eq) was treated with HF Pyridine (200 mL) at 0° C., which was followed by the addition of NaNO₂ (8.3 g, 119.8 mmol, 1.5 eq) in portions at 0° C. The resulting mixture was stirred for 1 hour at 0° C. The above mixture was slowly warmed to 25° C. The resulting mixture was stirred for overnight at 80° C. The reaction was quenched by the addition of water/ice (100 mL) at 0° C. and extracted with CH₂Cl₂ (3×50 mL). The combined organic layers were washed with brine (50 mL), dried over anhydrous Na₂SO₄. After filtration, the filtrate was concentrated under vacuum. The crude residue was applied onto a silica gel column and eluted with ethyl acetate/petroleum ether=1:5 to give 2-bromo-5-fluoro-6-iodo-3-methylpyridine as a colorless solid (21.0 g, 83.2%). ¹⁹FNMR: −106 ppm. ¹H NMR (400 MHz, Chloroform-d) δ 7.20 (d, J=7.1 Hz, 1H), 2.39 (s, 3H).

Synthesis of 2-bromo-5-fluoro-6-iodopyridine-3-carboxylic acid: Into a 1000 mL round-bottom flask were added 2-bromo-5-fluoro-6-iodo-3-methylpyridine (22.0 g, 69.6 mmol, 1.0 eq), Pyridine (200 mL) and H₂O (200 mL). The above mixture was added tetraoxo(potassio)manganese (110.1 g, 696.41 mmol, 10.0 eq) in portions. The resulting mixture was stirred for 12 hours at 100° C. The resulting mixture was filtered, the filter cake was washed with EtOAc (3×50 mL). The filtrate was concentrated under vacuum. The mixture was acidified to pH=1 with conc. HCl. The resulting mixture was extracted with EtOAc (3×50 mL). The combined organic layers were washed with brine (50 mL), dried over anhydrous Na₂SO₄. After filtration, the filtrate was concentrated under vacuum. Finally, 2-bromo-5-fluoro-6-iodopyridine-3-carboxylic acid was obtained as a colorless solid (8.0 g, 33.2%). ¹H NMR (300 MHz, DMSO-d₆) δ 14.12 (s, 1H), 8.05 (d, J=7.2 Hz, 1H).

Synthesis of methyl 2-bromo-5-fluoro-6-iodopyridine-3-carboxylate: Into a 250 mL round-bottom flask were added 2-bromo-5-fluoro-6-iodopyridine-3-carboxylic acid (8.2 g, 23.8 mmol, 1.0 eq), MeOH (40 mL), dioxane (40 mL) and TMSCHN₂ (10.8 g, 94.8 mmol, 4.0 eq) at 0° C. The resulting mixture was stirred for overnight at 25° C. The resulting mixture was concentrated under vacuum. The resulting mixture was diluted with water (30 mL) and extracted with EtOAc (3×30 mL). The combined organic layers were washed with brine (30 mL), dried over anhydrous Na₂SO₄. After filtration, the filtrate was concentrated under vacuum. The crude residue was applied onto a silica gel column and eluted with ethyl acetate/petroleum ether=1:3 to give methyl 2-bromo-5-fluoro-6-iodopyridine-3-carboxylate as an off-white solid (7.4 g, 86.7%). ¹H NMR (300 MHz, Chloroform-d) δ 7.72 (d, J=6.6 Hz, 1H), 3.99 (s, 3H).

Synthesis of methyl 2-bromo-5-fluoro-6-{2-oxo-7-azaspiro[3.5]nonan-7-yl}pyridine-3-carboxylate: Into a 250 mL round-bottom flask were added methyl 2-bromo-5-fluoro-6-iodopyridine-3-carboxylate (7.4 g, 20.5 mmol, 1.0 eq), 7-azaspiro[3.5]nonan-2-one (2.3 g, 16.4 mmol, 0.8 eq), Pd(PPh₃)₂Cl₂ (1.4 g, 2.1 mmol, 0.1 eq), Cs₂CO₃ (4.7 g, 61.7 mmol, 3.0 eq), RuPhos (1.9 g, 4.1 mmol, 0.2 eq) and dioxane (80 mL). The resulting mixture was stirred for 6 hours at 100° C. under nitrogen atmosphere. The resulting mixture was filtered, the filter cake was washed with EtOAc (3×30 mL). The filtrate was concentrated under vacuum and diluted with water (30 mL) and extracted with EtOAc (3×30 mL). The combined organic layers were washed with brine (30 mL), dried over anhydrous Na₂SO₄. After filtration, the filtrate was concentrated under vacuum. The crude residue was purified by reversed-phase flash chromatography using the following conditions: column, C18 silica gel; mobile phase, CH₃CN in water (0.1% TFA), 10% to 50% gradient in 10 min; detector, UV 254 nm. Finally, methyl 2-bromo-5-fluoro-6-{2-oxo-7-azaspiro[3.5]nonan-7-yl}pyridine-3-carboxylate was obtained as light yellow solid (2.3 g, 30.1%). LC-MS (ES, m/z) M+1: 371/373.

Synthesis of 2-bromo-5-fluoro-6-{2-oxo-7-azaspiro[3.5]nonan-7-yl}pyridine-3-carboxylic acid: Into a 40 mL vial were added methyl 2-bromo-5-fluoro-6-{2-oxo-7-azaspiro[3.5]nonan-7-yl}pyridine-3-carboxylate (600 mg, 1.6 mmol, 1.0 eq), dioxane (5 mL), MeOH (5 mL) and NaOH (4 M, 10 mL). The resulting mixture was stirred for 4 hours at 25° C. The resulting mixture was concentrated under vacuum. The resulting mixture was diluted with water (5 mL). The mixture was acidified to pH=3 with conc. HCl. The precipitated solids were collected by filtration and washed with water (3×3 mL). Finally, 2-bromo-5-fluoro-6-{2-oxo-7-azaspiro[3.5]nonan-7-yl}pyridine-3-carboxylic acid was obtained as an off-white solid (500 mg, 86.6%). LC-MS (ES, m/z) M+1: 357/359. ¹H NMR (400 MHz, DMSO-d₆) δ 13.10 (s, 1H), 7.83 (d, J=14.0 Hz, 1H), 3.71-3.60 (m, 4H), 2.91-2.88 (m, 4H), 1.90-1.70 (m, 4H).

Synthesis of 2-bromo-5-fluoro-N-[(3R)-5-nitro-3-(oxan-4-yl)-3,4-dihydro-2H-1,4-benzoxazin-7-ylsulfonyl]-6-{2-oxo-7-azaspiro[3.5]nonan-7-yl}pyridine-3-carboxamide: A solution of (3R)-5-nitro-3-(oxan-4-yl)-3,4-dihydro-2H-1,4-benzoxazine-7-sulfonamide (288 mg, 0.8 mmol, 1.0 eq) in DCM (6 mL) was treated with EDCl (209 mg, 1.1 mmol, 1.3 eq) and DMAP (113 mg, 0.9 mmol, 1.1 eq) for 10 min at 0° C., which was followed by the addition of 2-bromo-5-fluoro-6-{2-oxo-7-azaspiro[3.5]nonan-7-yl}pyridine-3-carboxylic acid (300 mg, 0.8 mmol, 1.0 eq) and TEA (212 mg, 2.1 mmol, 2.5 eq) in DCM (3 mL) dropwise at 25° C. The resulting mixture was stirred for 2 hours at 25° C. The resulting mixture was diluted with water (5 mL) and extracted with CH₂Cl₂ (3×10 mL). The combined organic layers were washed with brine (10 mL), dried over anhydrous Na₂SO₄. After filtration, the filtrate was concentrated under vacuum. The crude residue was purified by silica gel column chromatography, eluted with CH₂Cl₂/MeOH=30:1 to give 2-bromo-5-fluoro-N-[(3R)-5-nitro-3-(oxan-4-yl)-3,4-dihydro-2H-1,4-benzoxazin-7-ylsulfonyl]-6-{2-oxo-7-azaspiro[3.5]nonan-7-yl}pyridine-3-carboxamide as a yellow solid (450 mg, 78.5%). LC-MS (ES, m/z) M+1: 682/684.

Synthesis of 5-fluoro-N-[(3R)-5-nitro-3-(oxan-4-yl)-3,4-dihydro-2H-1,4-benzoxazin-7-ylsulfonyl]-6-{2-oxo-7-azaspiro[3.5]nonan-7-yl}-2-[(3R,8S)-15-{[2-(trimethylsilyl)ethoxy]methyl}-2,5-dioxa-9,15,17-triazatetracyclo[8.7.0.0\hat{ }{3,8}.0\hat{ }{12,16}]heptadeca-1(10),11,13,16-tetraen-9-yl]pyridine-3-carboxamide: Into a 40 mL vial were added 2-bromo-5-fluoro-N-[(3R)-5-nitro-3-(oxan-4-yl)-3,4-dihydro-2H-1,4-benzoxazin-7-ylsulfonyl]-6-{2-oxo-7-azaspiro[3.5]nonan-7-yl}pyridine-3-carboxamide (400 mg, 0.6 mmol, 1.0 eq), (3R,8S)-15-{[2-(trimethylsilyl)ethoxy]methyl}-2,5-dioxa-9,15,17-triazatetracyclo[8.7.0.0\hat{ }{3,8}.0\hat{ }{12,16}]heptadeca-1(10),11,13,16-tetraene (212 mg, 0.6 mmol, 1.0 eq), 1.10-phenanthroline (63 mg, 0.4 mmol, 0.6 eq), Cul (45 mg, 0.2 mmol, 0.4 eq), K₂CO₃ (243 mg, 1.8 mmol, 3.0 eq) and DMSO (8 mL). The resulting mixture was stirred for 2 hours at 100° C. under nitrogen atmosphere. The reaction was quenched by the addition of water (10 mL) and extracted with CH₂Cl₂ (3×10 mL). The combined organic layers were washed with brine (10 mL), dried over anhydrous Na₂SO₄. After filtration, the filtrate was concentrated under vacuum. The crude residue was purified by Prep-TLC (CH₂Cl₂/MeOH=15:1) to give 5-fluoro-N-[(3R)-5-nitro-3-(oxan-4-yl)-3,4-dihydro-2H-1,4-benzoxazin-7-ylsulfonyl]-6-{2-oxo-7-azaspiro[3.5]nonan-7-yl}-2-[(3R,8S)-15-{[2-(trimethylsilyl)ethoxy]methyl}-2,5-dioxa-9,15,17-triazatetracyclo[8.7.0.0\hat{ }{3,8}.0\hat{ }{12,16}]heptadeca-1(10),11,13,16-tetraen-9-yl]pyridine-3-carboxamide as a yellow solid (220 mg, 39.0%). LC-MS (ES, m/z) M+1: 963.

Synthesis of 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-5-fluoro-N-[(3R)-5-nitro-3-(oxan-4-yl)-3,4-dihydro-2H-1,4-benzoxazin-7-ylsulfonyl]-6-{2-oxo-7-azaspiro[3.5]nonan-7-yl}pyridine-3-carboxamide: Into an 8 mL vial were added 5-fluoro-N-[(3R)-5-nitro-3-(oxan-4-yl)-3,4-dihydro-2H-1,4-benzoxazin-7-ylsulfonyl]-6-{2-oxo-7-azaspiro[3.5]nonan-7-yl}-2-[(3R,8S)-15-{[2-(trimethylsilyl)ethoxy]methyl}-2,5-dioxa-9,15,17-triazatetracyclo[8.7.0.0\hat{ }{3,8}.0\hat{ }{12,16}]heptadeca-1(10),11,13,16-tetraen-9-yl]pyridine-3-carboxamide (200 mg, 0.2 mmol, 1.0 eq), ethylenediamine (250 mg, 4.2 mmol, 20.0 eq), TBAF (1086 mg, 4.2 mmol, 20.0 eq) and THE (3 mL). The resulting mixture was stirred for 4 hours at 80° C. The reaction was quenched by the addition of water (5 mL) and extracted with CH₂Cl₂ (3×5 mL). The combined organic layers were washed with brine (5 mL), dried over anhydrous Na₂SO₄. After filtration, the filtrate was concentrated under vacuum. The crude residue was purified by Prep-TLC (CH₂Cl₂/MeOH=13:1) to give 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-5-fluoro-N-[(3R)-5-nitro-3-(oxan-4-yl)-3,4-dihydro-2H-1,4-benzoxazin-7-ylsulfonyl]-6-{2-oxo-7-azaspiro[3.5]nonan-7-yl}pyridine-3-carboxamide as a yellow solid (70 mg, 40.5%). LC-MS (ES, m/z) M+1: 833.

Synthesis of 6-{2-[(2R)-4-[(3,4-difluorophenyl)methyl]-2-(2-isopropoxyphenyl)piperazin-1-yl]-7-azaspiro[3.5]nonan-7-yl}-2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-5-fluoro-N-[(3R)-5-nitro-3-(oxan-4-yl)-3,4-dihydro-2H-1,4-benzoxazin-7-ylsulfonyl]pyridine-3-carboxamide (assumed): Into an 8 mL vial were added (3R)-1-[(3,4-difluorophenyl)methyl]-3-(2-isopropoxyphenyl)piperazine (12.5 mg, 0.04 mmol, 1.0 eq), 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-5-fluoro-N-[(3R)-5-nitro-3-(oxan-4-yl)-3,4-dihydro-2H-1,4-benzoxazin-7-ylsulfonyl]-6-{2-oxo-7-azaspiro[3.5]nonan-7-yl}pyridine-3-carboxamide (30 mg, 0.04 mmol, 1.0 eq), NaBH₃CN (11.3 mg, 0.2 mmol, 5.0 eq), ZnCl₂ (24.5 mg, 0.2 mmol, 5.0 eq) and MeOH (5 mL). The resulting mixture was stirred for 4 hours at 70° C. The reaction was quenched by the addition of water (10 mL) and extracted with CH₂Cl₂ (3×10 mL). The combined organic layers were washed with brine (10 mL), dried over anhydrous Na₂SO₄. After filtration, the filtrate was concentrated under vacuum. The residue was purified by reversed-phase flash chromatography using the following conditions: column, C18 silica gel; mobile phase, MeCN in water (0.1% NH₃·H₂O), 10% to 50% gradient in 10 min; detector, UV 254 nm. Finally, 6-{2-[(2R)-4-[(3,4-difluorophenyl)methyl]-2-(2-isopropoxyphenyl)piperazin-1-yl]-7-azaspiro[3.5]nonan-7-yl}-2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-5-fluoro-N-[(3R)-5-nitro-3-(oxan-4-yl)-3,4-dihydro-2H-1,4-benzoxazin-7-ylsulfonyl]pyridine-3-carboxamide was obtained as a yellow solid (assumed) (13 mg, 31.0%). LC-MS (ES, m/z) M+1: 1164. ¹H NMR (400 MHz, Chloroform-d) δ 12.64 (s, 1H), 8.43 (d, J=22.7 Hz, 2H), 8.22 (d, J=2.2 Hz, 1H), 7.93 (d, J=14.0 Hz, 1H), 7.47-7.36 (m, 2H), 7.18 (dt, J=18.4, 8.6 Hz, 2H), 7.04 (d, J=23.1 Hz, 3H), 6.95-6.77 (m, 2H), 6.57 (s, 1H), 6.07 (s, 1H), 4.50-4.48 (m 3H), 4.18-3.96 (m, 4H), 3.96-3.73 (m, 2H), 3.66-3.32 (m, 10H), 3.05-2.82 (m, 3H), 2.62-2.53 (m, 1H), 2.32-2.29 (m, 2H), 2.24-2.03 (m, 2H), 1.97-1.75 (m, 3H), 1.75-1.52 (m, 12H), 1.52-1.38 (m, 5H)

### Example 235 Preparation of 6-{2-[(2R)-4-[(3,4-difluorophenyl)methyl]-2-(2-isopropoxyphenyl)piperazin-1-yl]-7-azaspiro[3.5]nonan-7-yl}-2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-5-fluoro-N-[(3R)-5-nitro-3-(oxan-4-yl)-3,4-dihydro-2H-1,4-benzoxazin-7-ylsulfonyl]pyridine-3-carboxamide was obtained as a yellow solid (assumed) (Rotamer 2)

Synthesis of 6-{2-[(2R)-4-[(3,4-difluorophenyl)methyl]-2-(2-isopropoxyphenyl)piperazin-1-yl]-7-azaspiro[3.5]nonan-7-yl}-2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-5-fluoro-N-[(3R)-5-nitro-3-(oxan-4-yl)-3,4-dihydro-2H-1,4-benzoxazin-7-ylsulfonyl]pyridine-3-carboxamide (assumed) (Rotamer 2): Into an 8 mL vial were added (3R)-1-[(3,4-difluorophenyl)methyl]-3-(2-isopropoxyphenyl)piperazine (12.5 mg, 0.04 mmol, 1.0 eq), 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-5-fluoro-N-[(3R)-5-nitro-3-(oxan-4-yl)-3,4-dihydro-2H-1,4-benzoxazin-7-ylsulfonyl]-6-{2-oxo-7-azaspiro[3.5]nonan-7-yl}pyridine-3-carboxamide (30 mg, 0.04 mmol, 1.0 eq), NaBH₃CN (11.3 mg, 0.2 mmol, 5.0 eq), ZnCl₂ (24.5 mg, 0.2 mmol, 5.0 eq) and MeOH (5 mL). The resulting mixture was stirred for 4 hours at 70° C. The reaction was quenched by the addition of water (10 mL) and extracted with CH₂Cl₂ (3×10 mL). The combined organic layers were washed with brine (10 mL), dried over anhydrous Na₂SO₄. After filtration, the filtrate was concentrated under vacuum. The residue was purified by reversed-phase flash chromatography using the following conditions: column, C18 silica gel; mobile phase, MeCN in water (0.1% NH₃·H₂O), 10% to 50% gradient in 10 min; detector, UV 254 nm. Finally, 6-{2-[(2R)-4-[(3,4-difluorophenyl)methyl]-2-(2-isopropoxyphenyl)piperazin-1-yl]-7-azaspiro[3.5]nonan-7-yl}-2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-5-fluoro-N-[(3R)-5-nitro-3-(oxan-4-yl)-3,4-dihydro-2H-1,4-benzoxazin-7-ylsulfonyl]pyridine-3-carboxamide was obtained as a yellow solid (assumed) (Rotamer 2) (5 mg, 11.9%). LC-MS (ES, m/z) M+1: 1164. ¹H NMR (400 MHz, Chloroform-d) δ 12.61 (s, 1H), 8.44 (s, 1H), 8.38-8.27 (m, 2H), 8.13 (d, J=9.1 Hz, 1H), 7.47 (d, J=2.1 Hz, 2H), 7.22-6.98 (m, 3H), 6.91-6.78 (m, 3H), 6.69 (s, 1H), 6.44 (d, J=2.4 Hz, 1H), 6.14 (d, J=3.4 Hz, 1H), 4.62-4.41 (m, 2H), 4.19-3.93 (m, 4H), 3.62-3.58 (m, 4H), 3.45-3.35 (m, 6H), 3.03-2.91 (m, 6H), 2.81-2.33 (m, 5H), 2.27 (s, 4H), 1.76-1.71 (m, 7H), 1.67-1.25 (m, 6H), 1.16-1.05 (m, 3H).

### Example 236 Preparation of 4-{2-[(2R)-4-[(3,4-difluorophenyl)methyl]-2-(2-isopropoxyphenyl)piperazin-1-yl]-7-azaspiro[3.5]nonan-7-yl}-2-[(3R,8S)-13-fluoro-2,5-dioxa-9,15,17-triazatetracyclo[8.7.0.0\hat{ }{3,8}.0\hat{ }{12,16}]heptadeca-1(10),11,13,16-tetraen-9-yl]-N-[(3R)-5-nitro-3-(oxan-4-yl)-3,4-dihydro-2H-1,4-benzoxazin-7-ylsulfonyl]benzamide

Synthesis of 2-bromo-4-{2-[(2R)-4-[(3,4-difluorophenyl)methyl]-2-(2-isopropoxyphenyl)piperazin-1-yl]-7-azaspiro[3.5]nonan-7-yl}-N-[(3R)-5-nitro-3-(oxan-4-yl)-3,4-dihydro-2H-1,4-benzoxazin-7-ylsulfonyl]benzamide: Into a 40-mL sealed tube, were placed (3R)-1-[(3,4-difluorophenyl)methyl]-3-(2-isopropoxyphenyl)piperazine (351 mg, 1.0 mmol, 1.2 eq), 2-bromo-N-[(3R)-5-nitro-3-(oxan-4-yl)-3,4-dihydro-2H-1,4-benzoxazin-7-ylsulfonyl]-4-{2-oxo-7-azaspiro[3.5]nonan-7-yl}benzamide (560 mg, 0.8 mmol, 1.0 eq), ZnCl₂ (345 mg, 2.5 mmol, 3.0 eq), NaBH₃CN (159 mg, 2.5 mmol, 3.0 eq), MeOH (6 mL). The resulting solution was stirred for overnight at 70° C. in an oil bath. The resulting mixture was then quenched by the addition of water (50 mL). The resulting solution was extracted with dichloromethane (2×50 mL) and washed with brine (2×50 mL) and the organic layers combined. The mixture was dried over anhydrous Na₂SO₄. After filtration, the filtrate was concentrated under vacuum. The crude residue was applied onto a silica gel column and eluted with dichloromethane/methanol=10:1 to give 2-bromo-4-{2-[(2R)-4-[(3,4-difluorophenyl)methyl]-2-(2-isopropoxyphenyl)piperazin-1-yl]-7-azaspiro[3.5]nonan-7-yl}-N-[(3R)-5-nitro-3-(oxan-4-yl)-3,4-dihydro-2H-1,4-benzoxazin-7-ylsulfonyl]benzamide as a yellow solid (600 mg, 71.5%). LC-MS (ESI, m/z) M+1: 993.

Synthesis of 4-{2-[(2R)-4-[(3,4-difluorophenyl)methyl]-2-(2-isopropoxyphenyl)piperazin-1-yl]-7-azaspiro[3.5]nonan-7-yl}-2-[(3R,8S)-13-fluoro-15-{[2-(trimethylsilyl)ethoxy]methyl}-2,5-dioxa-9,15,17-triazatetracyclo[8.7.0.0\hat{ }{3,8}.0\hat{ }{12,16}]heptadeca-1(10),11,13,16-tetraen-9-yl]-N-[(3R)-5-nitro-3-(oxan-4-yl)-3,4-dihydro-2H-1,4-benzoxazin-7-ylsulfonyl]benzamide: Into an 8-mL sealed tube purged and maintained with an inert atmosphere of nitrogen, were placed 2-bromo-4-{2-[(2R)-4-[(3,4-difluorophenyl)methyl]-2-(2-isopropoxyphenyl)piperazin-1-yl]-7-azaspiro[3.5]nonan-7-yl}-N-[(3R)-5-nitro-3-(oxan-4-yl)-3,4-dihydro-2H-1,4-benzoxazin-7-ylsulfonyl]benzamide (150 mg, 0.2 mmol, 1.0 eq), (3R,8S)-13-fluoro-15-{[2-(trimethylsilyl)ethoxy]methyl}-2,5-dioxa-9,15,17-triazatetracyclo[8.7.0.0\hat{ }{3,8}.0\hat{ }{12,16}]heptadeca-1(10),11,13,16-tetraene (46 mg, 0.1 mmol, 0.8 eq), Cul (17 mg, 0.1 mmol, 0.6 eq), t-BuONa (44 mg, 0.5 mmol, 3.0 eq), N,N′-bis(4-hydroxy-2,6-dimethylphenyl)ethanediamide (30 mg, 0.1 mmol, 0.6 eq), DMSO (1.5 mL). The resulting solution was stirred for 2 hours at 120° C. in an oil bath. The resulting mixture was then quenched by the addition of water (30 mL) and then extracted with ethyl acetate (2×30 mL) and washed with brine (2×30 mL) and the organic layers combined. The mixture was dried over anhydrous Na₂SO₄. After filtration, the filtrate was concentrated under vacuum. The crude residue was applied onto a silica gel column and eluted with dichloromethane/methanol=20:1 to give 4-{2-[(2R)-4-[(3,4-difluorophenyl)methyl]-2-(2-isopropoxyphenyl)piperazin-1-yl]-7-azaspiro[3.5]nonan-7-yl}-2-[(3R,8S)-13-fluoro-15-{[2-(trimethylsilyl)ethoxy]methyl}-2,5-dioxa-9,15,17-triazatetracyclo[8.7.0.0\hat{ }{3,8}.0\hat{ }{12,16}]heptadeca-1(10),11,13,16-tetraen-9-yl]-N-[(3R)-5-nitro-3-(oxan-4-yl)-3,4-dihydro-2H-1,4-benzoxazin-7-ylsulfonyl]benzamide as a yellow solid (35 mg, 17.9%). LC-MS (ESI, m/z) M+1: 1293.

Synthesis of 4-{2-[(2R)-4-[(3,4-difluorophenyl)methyl]-2-(2-isopropoxyphenyl)piperazin-1-yl]-7-azaspiro[3.5]nonan-7-yl}-2-[(3R,8S)-13-fluoro-2,5-dioxa-9,15,17-triazatetracyclo[8.7.0.0\hat{ }{3,8}.0\hat{ }{12,16}]heptadeca-1(10),11,13,16-tetraen-9-yl]-N-[(3R)-5-nitro-3-(oxan-4-yl)-3,4-dihydro-2H-1,4-benzoxazin-7-ylsulfonyl]benzamide: Into an 8-mL sealed tube, were placed 4-{2-[(2R)-4-[(3,4-difluorophenyl)methyl]-2-(2-isopropoxyphenyl)piperazin-1-yl]-7-azaspiro[3.5]nonan-7-yl}-2-[(3R,8S)-13-fluoro-15-{[2-(trimethylsilyl)ethoxy]methyl}-2,5-dioxa-9,15,17-triazatetracyclo[8.7.0.0\hat{ }{3,8}.0\hat{ }{12,16}]heptadeca-1(10),11,13,16-tetraen-9-yl]-N-[(3R)-5-nitro-3-(oxan-4-yl)-3,4-dihydro-2H-1,4-benzoxazin-7-ylsulfonyl]benzamide (30 mg, 0.02 mmol, 1.0 eq), ethylenediamine (28 mg, 0.5 mmol, 20.0 eq), TBAF in THF(1.0 M) (1 mL). The resulting solution was stirred for 6 hours at 50° C. in an oil bath. The resulting mixture was then quenched by the addition of water (50 mL). The resulting solution was extracted with ethyl acetate (2×50 mL) and washed with brine (2×50 mL) and the organic layers combined. The mixture was dried over anhydrous Na₂SO₄. After filtration, the filtrate was concentrated under vacuum. The crude product was purified by Flash-Prep-HPLC using the following conditions: Column: Xselect CSH C18 OBD Column 30*150 mm 5 μm, n; Mobile Phase A: water (0.1% FA), Mobile Phase B: CH₃CN; Flow rate: 60 mL/min; Gradient: 30% B to 50% B in 8 min, 50% B; Wave Length: 220/254 nm; RT1(min): 6.80; Number Of Runs: 3. Finally, 4-{2-[(2R)-4-[(3,4-difluorophenyl)methyl]-2-(2-isopropoxyphenyl)piperazin-1-yl]-7-azaspiro[3.5]nonan-7-yl}-2-[(3R,8S)-13-fluoro-2,5-dioxa-9,15,17-triazatetracyclo[8.7.0.0\hat{ }{3,8}.0\hat{ }{12,16}]heptadeca-1(10),11,13,16-tetraen-9-yl]-N-[(3R)-5-nitro-3-(oxan-4-yl)-3,4-dihydro-2H-1,4-benzoxazin-7-ylsulfonyl]benzamide was obtained as a yellow solid (5 mg, 18.5%). LC-MS (ESI, m/z) M+1: 1162. ¹H NMR (400 MHz, Methanol-d₄) δ 8.02-7.97 (m, 1H), 7.83-7.76 (m, 1H), 7.49-7.41 (m, 1H), 7.35-7.23 (m, 3H), 7.22-7.11 (m, 2H), 7.04-6.88 (m, 3H), 6.82-6.77 (m, 1H), 6.73-6.65 (m, 1H), 6.46-6.35 (m, 1H), 4.30-4.11 (m, 5H), 4.05-3.86 (m, 2H), 3.72-3.55 (m, 4H), 3.53-3.38 (m, 4H), 3.23-3.06 (m, 6H), 3.05-2.96 (m, 1H), 2.76-2.56 (m, 2H), 2.51-2.37 (m, 2H), 2.13-1.82 (m, 4H), 1.81-1.68 (m, 3H), 1.66-1.42 (m, 6H), 1.41-1.34 (m, 5H), 1.32-1.24 (m, 5H).

### Example 239 Preparation of 4-{2-[(2R)-4-[(3,4-difluorophenyl)methyl]-2-(2-isopropoxyphenyl)piperazin-1-yl]-7-azaspiro[3.5]nonan-7-yl}-2-[(3S,8R)-2,5-dioxa-9,15,17-triazatetracyclo[8.7.0.0\hat{ }{3,8}.0\hat{ }{12,16}]heptadeca-1(10),11,13,16-tetraen-9-yl]-N-[(3R)-5-nitro-3-(oxan-4-yl)-3,4-dihydro-2H-1,4-benzoxazin-7-ylsulfonyl]benzamide

Synthesis of 4-{2-[(2R)-4-[(3,4-difluorophenyl)methyl]-2-(2-isopropoxyphenyl)piperazin-1-yl]-7-azaspiro[3.5]nonan-7-yl}-N-[(3R)-5-nitro-3-(oxan-4-yl)-3,4-dihydro-2H-1,4-benzoxazin-7-ylsulfonyl]-2-[(3S,8R)-15-{[2-(trimethylsilyl)ethoxy]methyl}-2,5-dioxa-9,15,17-triazatetracyclo[8.7.0.0\hat{ }{3,8}.0\hat{ }{12,16}]heptadeca-1(10),11,13,16-tetraen-9-yl]benzamide: Into an 8 mL vial were added 2-bromo-4-{2-[(2R)-4-[(3,4-difluorophenyl)methyl]-2-(2-isopropoxyphenyl)piperazin-1-yl]-7-azaspiro[3.5]nonan-7-yl}-N-[(3R)-5-nitro-3-(oxan-4-yl)-3,4-dihydro-2H-1,4-benzoxazin-7-ylsulfonyl]benzamide (120 mg, 0.1 mmol, 1.0 eq), (3S,8R)-15-{[2-(trimethylsilyl)ethoxy]methyl}-2,5-dioxa-9,15,17-triazatetracyclo[8.7.0.0\hat{ }{3,8}.0\hat{ }{12,16}]heptadeca-1(10),11,13,16-tetraene (44 mg, 0.1 mmol, 1.0 eq), ethanediamide, N,N′-diphenyl- (17 mg, 0.07 mmol, 0.6 eq), Cul (9 mg, 0.05 mmol, 0.4 eq), K₂CO₃ (50 mg, 0.4 mmol, 3.0 eq) and DMSO (3 mL). The resulting mixture was stirred for 2 hours at 120° C. under nitrogen atmosphere. The reaction was quenched by the addition of water (3 mL) and extracted with CH₂Cl₂ (3×5 mL). The combined organic layers were washed with brine (5 mL), dried over anhydrous Na₂SO₄. After filtration, the filtrate was concentrated under vacuum. The residue was purified by Prep-TLC (CH₂Cl₂/MeOH 15:1) to give 4-{2-[(2R)-4-[(3,4-difluorophenyl)methyl]-2-(2-isopropoxyphenyl)piperazin-1-yl]-7-azaspiro[3.5]nonan-7-yl}-N-[(3R)-5-nitro-3-(oxan-4-yl)-3,4-dihydro-2H-1,4-benzoxazin-7-ylsulfonyl]-2-[(3S,8R)-15-{[2-(trimethylsilyl)ethoxy]methyl}-2,5-dioxa-9,15,17-triazatetracyclo[8.7.0.0\hat{ }{3,8}.0\hat{ }{12,16}]heptadeca-1(10),11,13,16-tetraen-9-yl]benzamide as an orange solid (60 mg, 39.0%). LC-MS (ESI, m/z) M+1: 1275.

Synthesis of 4-{2-[(2R)-4-[(3,4-difluorophenyl)methyl]-2-(2-isopropoxyphenyl)piperazin-1-yl]-7-azaspiro[3.5]nonan-7-yl}-2-[(3S,8R)-2,5-dioxa-9,15,17-triazatetracyclo[8.7.0.0\hat{ }{3,8}.0\hat{ }{12,16}]heptadeca-1(10),11,13,16-tetraen-9-yl]-N-[(3R)-5-nitro-3-(oxan-4-yl)-3,4-dihydro-2H-1,4-benzoxazin-7-ylsulfonyl]benzamide: Into an 8 mL vial were added 4-{2-[(2R)-4-[(3,4-difluorophenyl)methyl]-2-(2-isopropoxyphenyl)piperazin-1-yl]-7-azaspiro[3.5]nonan-7-yl}-N-[(3R)-5-nitro-3-(oxan-4-yl)-3,4-dihydro-2H-1,4-benzoxazin-7-ylsulfonyl]-2-[(3S,8R)-15-{[2-(trimethylsilyl)ethoxy]methyl}-2,5-dioxa-9,15,17-triazatetracyclo[8.7.0.0\hat{ }{3,8}.0\hat{ }{12,16}]heptadeca-1(10),11,13,16-tetraen-9-yl]benzamide (55 mg, 0.04 mmol, 1.0 eq) and TBAF (226 mg, 0.9 mmol, 20.0 eq). The resulting mixture was stirred for 4 hours at 80° C. The reaction was quenched by the addition of water (3 mL) and extracted with CH₂Cl₂ (3×5 mL). The combined organic layers were washed with brine (5 mL), dried over anhydrous Na₂SO₄. After filtration, the filtrate was concentrated under vacuum. The residue was dissolved in EtOAc (20 mL) and washed with 3×10 mL of water, dried over anhydrous Na₂SO₄. After filtration, the filtrate was concentrated under vacuum. The residue was purified by reversed-phase flash chromatography using the following conditions: column, C18 silica gel; mobile phase, MeCN in water (0.1% NH₃·H₂O), 10% to 50% gradient in 10 min; detector, UV 254 nm. Finally, 4-{2-[(2R)-4-[(3,4-difluorophenyl)methyl]-2-(2-isopropoxyphenyl)piperazin-1-yl]-7-azaspiro[3.5]nonan-7-yl}-2-[(3S,8R)-2,5-dioxa-9,15,17-triazatetracyclo[8.7.0.0\hat{ }{3,8}.0\hat{ }{12,16}]heptadeca-1(10),11,13,16-tetraen-9-yl]-N-[(3R)-5-nitro-3-(oxan-4-yl)-3,4-dihydro-2H-1,4-benzoxazin-7-ylsulfonyl]benzamide was obtained as yellow solid (13 mg, 26.3%). LC-MS (ESI, m/z) M+1: 1144. ¹H NMR (400 MHz, Chloroform-d) δ 12.62 (s, 1H), 8.44 (s, 1H), 8.38 (s, 1H), 8.30 (d, J=2.3 Hz, 1H), 8.13 (d, J=9.2 Hz, 1H), 7.49-7.42 (m, 2H), 7.18 (dt, J=16.6, 8.7 Hz, 2H), 7.12-6.97 (m, 3H), 6.90-6.78 (m, 3H), 6.68 (d, J=9.1 Hz, 1H), 6.44 (s, 1H), 6.14 (s, 1H), 4.48-4.45 (m, 3H), 4.22-4.03 (m, 4H), 4.03-3.83 (m, 2H), 3.60-3.57 (m, 1H), 3.45-3.36 (m, 7H), 3.09-3.14 (m, 4H), 3.03-2.82 (m, 3H), 2.68-2.52 (m, 2H), 2.32-2.28 (m, 2H), 2.22-2.01 (m, 2H), 1.80-1.72 (m, 4H), 1.63-1.56 (m, 3H), 1.54-1.37 (m, 5H), 1.33-1.27 (m, 6H).

### Example 240 Preparation of 4-{2-[(2R)-4-[(3,4-difluorophenyl)methyl]-2-(2-isopropoxyphenyl)piperazin-1-yl]-7-azaspiro[3.5]nonan-7-yl}-2-[(3S,8S)-5,9-dioxa-2,11,13-triazatetracyclo[8.7.0.0\hat{ }{3,8}.0\hat{ }{12,16}]heptadeca-1(10),11,14,16-tetraen-2-yl]-N-[(3R)-5-nitro-3-(oxan-4-yl)-3,4-dihydro-2H-1,4-benzoxazin-7-ylsulfonyl]benzamide

Synthesis of 4-{2-[(2R)-4-[(3,4-difluorophenyl)methyl]-2-(2-isopropoxyphenyl)piperazin-1-yl]-7-azaspiro[3.5]nonan-7-yl}-N-[(3R)-5-nitro-3-(oxan-4-yl)-3,4-dihydro-2H-1,4-benzoxazin-7-ylsulfonyl]-2-[(3S,8S)-13-{[2-(trimethylsilyl)ethoxy]methyl}-5,9-dioxa-2,11,13-triazatetracyclo[8.7.0.0\hat{ }{3,8}.0\hat{ }{12,16}]heptadeca-1(10),11,14,16-tetraen-2-yl]benzamide: Into an 8-mL sealed tube purged and maintained with an inert atmosphere of nitrogen, were placed 2-bromo-4-{2-[(2R)-4-[(3,4-difluorophenyl)methyl]-2-(2-isopropoxyphenyl)piperazin-1-yl]-7-azaspiro[3.5]nonan-7-yl}-N-[(3R)-5-nitro-3-(oxan-4-yl)-3,4-dihydro-2H-1,4-benzoxazin-7-ylsulfonyl]benzamide (140 mg, 0.1 mmol, 1.0 eq), (3S,8S)-13-{[2-(trimethylsilyl)ethoxy]methyl}-5,9-dioxa-2,11,13-triazatetracyclo[8.7.0.0\hat{ }{3,8}.0\hat{ }{12,16}]heptadeca-1(10),11,14,16-tetraene (41 mg, 0.1 mmol, 0.8 eq), Cul (16 mg, 0.1 mmol, 0.6 eq), t-BuONa (41 mg, 0.4 mmol, 3.0 eq), N,N′-bis(thiophen-2-ylmethyl)ethanediamide (24 mg, 0.1 mmol, 0.6 eq), DMSO (1.5 mL). The resulting solution was stirred for 2 hours at 100° C. in an oil bath. The resulting mixture was then quenched by the addition of water (30 mL). The resulting solution was extracted with ethyl acetate (2×30 mL) and washed with brine (2×30 mL) and the organic layers combined. The mixture was dried over anhydrous Na₂SO₄. After filtration, the filtrate was concentrated under vacuum. The crude residue was applied onto a silica gel column and eluted with dichloromethane/methanol=20:1 to give 4-{2-[(2R)-4-[(3,4-difluorophenyl)methyl]-2-(2-isopropoxyphenyl)piperazin-1-yl]-7-azaspiro[3.5]nonan-7-yl}-N-[(3R)-5-nitro-3-(oxan-4-yl)-3,4-dihydro-2H-1,4-benzoxazin-7-ylsulfonyl]-2-[(3S,8S)-13-{[2-(trimethylsilyl)ethoxy]methyl}-5,9-dioxa-2,11,13-triazatetracyclo[8.7.0.0\hat{ }{3,8}.0\hat{ }{12,16}]heptadeca-1(10),11,14,16-tetraen-2-yl]benzamide as a yellow solid (60 mg, 33.4%). LC-MS (ESI, m/z) M+1: 1275.

Synthesis of 4-{2-[(2R)-4-[(3,4-difluorophenyl)methyl]-2-(2-isopropoxyphenyl)piperazin-1-yl]-7-azaspiro[3.5]nonan-7-yl}-2-[(3S,8S)-5,9-dioxa-2,11,13-triazatetracyclo[8.7.0.0\hat{ }{3,8}.0\hat{ }{12,16}]heptadeca-1(10),11,14,16-tetraen-2-yl]-N-[(3R)-5-nitro-3-(oxan-4-yl)-3,4-dihydro-2H-1,4-benzoxazin-7-ylsulfonyl]benzamide: Into an 8-mL sealed tube, were placed 4-{2-[(2R)-4-[(3,4-difluorophenyl)methyl]-2-(2-isopropoxyphenyl)piperazin-1-yl]-7-azaspiro[3.5]nonan-7-yl}-N-[(3R)-5-nitro-3-(oxan-4-yl)-3,4-dihydro-2H-1,4-benzoxazin-7-ylsulfonyl]-2-[(3S,8S)-13-{[2-(trimethylsilyl)ethoxy]methyl}-5,9-dioxa-2,11,13-triazatetracyclo[8.7.0.0\hat{ }{3,8}.0\hat{ }{12,16}]heptadeca-1(10),11,14,16-tetraen-2-yl]benzamide (60 mg, 0.05 mmol, 1.0 eq), ethylenediamine (57 mg, 0.9 mmol, 20.0 eq), TBAF in THF(1.0 M) (1 mL). The resulting solution was stirred for 6 hours at 80° C. in an oil bath. The resulting mixture was then quenched by the addition of water (50 mL), and then extracted with ethyl acetate (2×50 mL). The combined organic layers was washed with brine (2×50 mL), dried over anhydrous Na₂SO₄. After filtration, the filtrate was concentrated under vacuum. The crude product was purified by Flash-Prep-HPLC using the following conditions: Column: XBridge Shield RP18 OBD Column, 30*150 mm, 5 μm; Mobile Phase A: water (0.05% NH₃·H₂O), Mobile Phase B: CH₃CN; Flow rate: 60 mL/min; Gradient: 25% B to 45% B in 8 min, Wave Length: 254/220 nm. Finally, 4-{2-[(2R)-4-[(3,4-difluorophenyl)methyl]-2-(2-isopropoxyphenyl)piperazin-1-yl]-7-azaspiro[3.5]nonan-7-yl}-2-[(3S,8S)-5,9-dioxa-2,11,13-triazatetracyclo[8.7.0.0\hat{ }{3,8}.0\hat{ }{12,16}]heptadeca-1(10),11,14,16-tetraen-2-yl]-N-[(3R)-5-nitro-3-(oxan-4-yl)-3,4-dihydro-2H-1,4-benzoxazin-7-ylsulfonyl]benzamide was obtained as a yellow solid (10 mg, 18.6%). LC-MS (ESI, m/z) M+1: 1144. ¹H NMR (400 MHz, Methanol-d₄) δ 8.13-8.00 (m 1H), 7.76-7.59 (m, 1H), 7.51-7.40 (m, 1H), 7.39-7.11 (m, 5H), 7.01-6.86 (m, 4H), 6.74-6.69 (m, 1H), 6.53-6.47 (m, 1H), 5.99-5.93 (m, 1H), 4.68-4.56 (m, 1H), 4.40-4.33 (m, 1H), 4.23-3.97 (m, 6H), 3.77-3.36 (m, 9H), 3.20-3.01 (m, 5H), 3.00-2.91 (m, 1H), 2.73-2.61 (m, 1H), 2.45-2.24 (m, 4H), 2.02-1.67 (m, 6H), 1.55-1.39 (m, 6H), 1.38-1.21 (m, 9H).

### Example 241 Preparation of 4-{2-[(2R)-4-[(3,4-difluorophenyl)methyl]-2-(2-isopropoxyphenyl)piperazin-1-yl]-7-azaspiro[3.5]nonan-7-yl}-2-[(3R,8R)-5,9-dioxa-2,11,13-triazatetracyclo[8.7.0.0\hat{ }{3,8}.0\hat{ }{12,16}]heptadeca-1(10),11,14,16-tetraen-2-yl]-N-[(3R)-5-nitro-3-(oxan-4-yl)-3,4-dihydro-2H-1,4-benzoxazin-7-ylsulfonyl]benzamide

Synthesis of 4-{2-[(2R)-4-[(3,4-difluorophenyl)methyl]-2-(2-isopropoxyphenyl)piperazin-1-yl]-7-azaspiro[3.5]nonan-7-yl}-N-[(3R)-5-nitro-3-(oxan-4-yl)-3,4-dihydro-2H-1,4-benzoxazin-7-ylsulfonyl]-2-[(3R,8R)-13-{[2-(trimethylsilyl)ethoxy]methyl}-5,9-dioxa-2,11,13-triazatetracyclo[8.7.0.0\hat{ }{3,8}.0\hat{ }{12,16}]heptadeca-1(10),11,14,16-tetraen-2-yl]benzamide: Into an 8-mL sealed tube purged and maintained with an inert atmosphere of nitrogen, were placed 2-bromo-4-{2-[(2R)-4-[(3,4-difluorophenyl)methyl]-2-(2-isopropoxyphenyl)piperazin-1-yl]-7-azaspiro[3.5]nonan-7-yl}-N-[(3R)-5-nitro-3-(oxan-4-yl)-3,4-dihydro-2H-1,4-benzoxazin-7-ylsulfonyl]benzamide (110 mg, 0.1 mmol, 1.0 eq), (3R,8R)-13-{[2-(trimethylsilyl)ethoxy]methyl}-5,9-dioxa-2,11,13-triazatetracyclo[8.7.0.0\hat{ }{3,8}.0\hat{ }{12,16}]heptadeca-1(10),11,14,16-tetraene (32 mg, 0.09 mmol, 0.8 eq), Cul (13 mg, 0.07 mmol, 0.6 eq), t-BuONa (32 mg, 0.3 mmol, 3.0 eq), N,N′-bis(thiophen-2-ylmethyl)ethanediamide (19 mg, 0.07 mmol, 0.6 eq), DMSO (1.2 mL). The resulting solution was stirred for 2 hours at 100° C. in an oil bath. The resulting mixture was then quenched by the addition of water (30 mL), and then extracted with ethyl acetate (2×30 mL). The combined organic layers was washed with brine (2×30 mL) and the organic layers combined. The mixture was dried over anhydrous Na₂SO₄. After filtration, the filtrate was concentrated under vacuum. The crude residue was applied onto a silica gel column and eluted with dichloromethane/methanol=20:1 to give 4-{2-[(2R)-4-[(3,4-difluorophenyl)methyl]-2-(2-isopropoxyphenyl)piperazin-1-yl]-7-azaspiro[3.5]nonan-7-yl}-N-[(3R)-5-nitro-3-(oxan-4-yl)-3,4-dihydro-2H-1,4-benzoxazin-7-ylsulfonyl]-2-[(3R,8R)-13-{[2-(trimethylsilyl)ethoxy]methyl}-5,9-dioxa-2,11,13-triazatetracyclo[8.7.0.0\hat{ }{3,8}.0\hat{ }{12,16}]heptadeca-1(10),11,14,16-tetraen-2-yl]benzamide as a yellow solid (35 mg, 24.8%). LC-MS (ESI, m/z) M+1: 1275.

Synthesis of 4-{2-[(2R)-4-[(3,4-difluorophenyl)methyl]-2-(2-isopropoxyphenyl)piperazin-1-yl]-7-azaspiro[3.5]nonan-7-yl}-2-[(3R,8R)-5,9-dioxa-2,11,13-triazatetracyclo[8.7.0.0\hat{ }{3,8}.0\hat{ }{12,16}]heptadeca-1(10),11,14,16-tetraen-2-yl]-N-[(3R)-5-nitro-3-(oxan-4-yl)-3,4-dihydro-2H-1,4-benzoxazin-7-ylsulfonyl]benzamide: Into an 8-mL sealed tube, were placed 4-{2-[(2R)-4-[(3,4-difluorophenyl)methyl]-2-(2-isopropoxyphenyl)piperazin-1-yl]-7-azaspiro[3.5]nonan-7-yl}-N-[(3R)-5-nitro-3-(oxan-4-yl)-3,4-dihydro-2H-1,4-benzoxazin-7-ylsulfonyl]-2-[(3R,8R)-13-{[2-(trimethylsilyl)ethoxy]methyl}-5,9-dioxa-2,11,13-triazatetracyclo[8.7.0.0\hat{ }{3,8}.0\hat{ }{12,16}]heptadeca-1(10),11,14,16-tetraen-2-yl]benzamide (35 mg, 0.03 mmol, 1.0 eq), ethylenediamine (33 mg, 0.5 mmol, 20.0 eq), TBAF in THF(1.0 M, 1 mL). The resulting solution was stirred for 4 hours at 80° C. in an oil bath. The resulting mixture was then quenched by the addition of water (50 mL), and then extracted with ethyl acetate (2×50 mL). The combined organic layers was washed with brine (2×50 mL), dried over anhydrous Na₂SO₄. After filtration, the filtrate was concentrated under vacuum. The crude product was purified by Flash-Prep-HPLC using the following conditions: Column: XBridge Shield RP18 OBD Column, 30*150 mm, 5 μm; Mobile Phase A: water (0.05% NH₃·H₂O), Mobile Phase B: CH₃CN; Flow rate: 60 mL/min; Gradient: 25% B to 45% B in 8 min, Wave Length: 254/220 nm; Finally, 4-{2-[(2R)-4-[(3,4-difluorophenyl)methyl]-2-(2-isopropoxyphenyl)piperazin-1-yl]-7-azaspiro[3.5]nonan-7-yl}-2-[(3R,8R)-5,9-dioxa-2,11,13-triazatetracyclo[8.7.0.0\hat{ }{3,8}.0\hat{ }{12,16}]heptadeca-1(10),11,14,16-tetraen-2-yl]-N-[(3R)-5-nitro-3-(oxan-4-yl)-3,4-dihydro-2H-1,4-benzoxazin-7-ylsulfonyl]benzamide was obtained as a yellow solid (8 mg, 25.5%). LC-MS (ESI, m/z) M+1: 1144. ¹H NMR (400 MHz, Methanol-d₄) δ 8.13-8.00 (m 1H), 7.76-7.59 (m, 1H), 7.51-7.40 (m, 1H), 7.39-7.11 (m, 5H), 7.01-6.86 (m, 4H), 6.74-6.69 (m, 1H), 6.53-6.47 (m, 1H), 5.99-5.93 (m, 1H), 4.69-4.57 (m, 1H), 4.40-4.15 (m, 3H), 4.14-3.95 (m, 4H), 3.74-3.53 (m, 5H), 3.48-3.25 (m, 4H), 3.18-3.05 (m, 4H), 3.01-2.95 (m, 1H), 2.75-2.65 (m, 3H), 2.49-2.24 (m, 3H), 2.08-1.67 (m, 6H), 1.57-1.37 (m, 7H), 1.35-1.24 (m, 8H).

### Example 242 Preparation of 4-{2-[(2R)-4-[(1R)-1-(3,4-difluorophenyl)ethyl]-2-(2-isopropoxyphenyl)piperazin-1-yl]-7-azaspiro[3.5]nonan-7-yl}-2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-N-[(3R)-5-nitro-3-(oxan-4-yl)-3,4-dihydro-2H-1,4-benzoxazin-7-ylsulfonyl]benzamide (assumed)

Synthesis of 1-(3,4-difluorophenyl)ethanol: Into a 100 mL 3-necked round-bottom, were placed 1-(3,4-difluorophenyl)ethanone (3.0 g, 19.2 mmol, 1.0 eq), MeOH (30 mL) at 0° C. To the above mixture was added NaBH₄ (1.4 g, 38.4 mmol, 2.0 eq) at 0° C. The resulting mixture was stirred for additional 2 hours at 25° C. The resulting mixture was then quenched by the addition of water (50 mL), and then extracted with ethyl acetate (2×50 mL). The combined organic layer was washed with brine (2×50 mL), dried over anhydrous Na₂SO₄. After filtration, the filtrate was concentrated under vacuum. The crude residue was purified onto a silica gel column and eluted with ethyl acetate/petroleum ether=1:3 to give 1-(3,4-difluorophenyl)ethanol as a colorless oil (2.5 g, 82.3%). ¹H NMR (300 MHz, DMSO-d₆) δ 7.43-7.28 (m, 2H), 7.22-7.13 (m, 1H), 5.33 (d, J=4.5 Hz, 1H), 4.72 (dd, J=6.6, 4.5 Hz, 1H), 1.31 (d, J=6.6 Hz, 3H).

Synthesis of 4-(1-chloroethyl)-1,2-difluorobenzene: Into a 40 mL sealed tube, were placed 1-(3,4-difluorophenyl)ethanol (500 mg, 3.2 mmol, 1.0 eq), CHCl₃ (2 mL) at 0° C. To the above mixture was added SOCl₂ (3 mL) at 0° C. The resulting mixture was stirred for additional 4 hours at 25° C. The resulting mixture was then quenched by the addition of water (50 mL). The mixture was extracted with dichloromethane (2×50 mL) and washed with brine (2×50 mL). The mixture was dried over anhydrous Na₂SO₄. After filtration, the filtrate was concentrated under vacuum. The crude residue was purified onto a silica gel column and eluted with ethyl acetate/petroleum ether=1:3 to give 4-(1-chloroethyl)-1,2-difluorobenzene as a light yellow oil (300 mg, 53.7%). ¹H NMR (300 MHz, DMSO-d₆) δ 7.60 (ddd, J=11.7, 7.8, 2.1 Hz, 1H), 7.50-7.31 (m, 2H), 5.36 (q, J=6.9 Hz, 1H), 1.78 (d, J=6.9 Hz, 3H).

Synthesis of (3R)-1-[(1R)-1-(3,4-difluorophenyl)ethyl]-3-(2-isopropoxyphenyl)piperazine (assumed) & (3R)-1-[(1S)-1-(3,4-difluorophenyl)ethyl]-3-(2-isopropoxyphenyl)piperazine (assumed): Into an 8-mL sealed tube, were placed 4-(1-chloroethyl)-1,2-difluorobenzene (289 mg, 1.6 mmol, 1.2 eq), (2R)-2-(2-isopropoxyphenyl)piperazine (300 mg, 1.4 mmol, 1.0 eq), K₂CO₃ (565 mg, 4.1 mmol, 3.0 eq), CH₃CN (3 mL). The resulting solution was stirred for overnight at 80° C. The resulting mixture was then quenched by the addition of water (30 mL). The resulting solution was extracted with dichloromethane (2×30 mL) and washed with brine (2×30 mL) and the organic layers combined. The mixture was dried over anhydrous Na₂SO₄. After filtration, the filtrate was concentrated under vacuum. The crude residue was applied onto a silica gel column and eluted with ethyl acetate/petroleum ether=1:0 to give (3R)-1-[(1R)-1-(3,4-difluorophenyl)ethyl]-3-(2-isopropoxyphenyl)piperazine (assumed) as a light yellow oil (80 mg, 16.3%) and (3R)-1-[(1S)-1-(3,4-difluorophenyl)ethyl]-3-(2-isopropoxyphenyl)piperazine (assumed) as a light yellow oil (50 mg, 10.2%). ¹H NMR_A (300 MHz, Chloroform-d) δ 7.47 (dd, J=7.5, 1.8 Hz, 1H), 7.30-7.16 (m, 2H), 7.09 (dd, J=9.3, 6.6 Hz, 2H), 6.98-6.83 (m, 2H), 4.67-4.50 (m, 1H), 4.25 (dd, J=9.9, 2.7 Hz, 1H), 3.45 (q, J=6.6 Hz, 1H), 3.10-3.03 (m, 3H), 2.76-2.63 (m, 1H), 2.28-2.20 (m, 1H), 2.01 (t, J=10.2 Hz, 1H), 1.40-1.28 (m, 9H). ¹H NMR_B (300 MHz, Chloroform-d) δ 7.43 (dd, J=7.5, 1.8 Hz, 1H), 7.27-7.12 (m, 2H), 7.11-6.96 (m, 2H), 6.88 (t, J=7.5 Hz, 1H), 6.80 (d, J=8.1 Hz, 1H), 4.56-4.50 (m, 1H), 4.18 (dd, J=9.9, 2.7 Hz, 1H), 3.32 (q, J=6.6 Hz, 1H), 3.24-3.01 (m, 3H), 3.00-2.86 (m, 1H), 2.28-2.20 (m, 1H), 1.89-1.77 (m, 2H), 1.33 (dd, J=10.5, 6.3 Hz, 6H), 1.14 (d, J=6.0 Hz, 3H).

Synthesis of 4-{2-[(2R)-4-[(1R)-1-(3,4-difluorophenyl)ethyl]-2-(2-isopropoxyphenyl)piperazin-1-yl]-7-azaspiro[3.5]nonan-7-yl}-2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-N-[(3R)-5-nitro-3-(oxan-4-yl)-3,4-dihydro-2H-1,4-benzoxazin-7-ylsulfonyl]benzamide (assumed): Into an 8-mL sealed tube, were placed 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-N-[(3R)-5-nitro-3-(oxan-4-yl)-3,4-dihydro-2H-1,4-benzoxazin-7-ylsulfonyl]-4-{2-oxo-7-azaspiro[3.5]nonan-7-yl}benzamide (20 mg, 0.02 mmol, 1.0 eq), (3R)-1-[(1R)-1-(3,4-difluorophenyl)ethyl]-3-(2-isopropoxyphenyl)piperazine (assumed) (11 mg, 0.03 mmol, 1.2 eq), ZnCl₂ (10 mg, 0.08 mmol, 3.0 eq), NaBH₃CN (5 mg, 0.08 mmol, 3.0 eq), MeOH (0.5 mL). The resulting solution was stirred for overnight at 70° C. in an oil bath. The crude product was purified by Flash-Prep-HPLC using the following conditions: Column: XBridge Shield RP18 OBD Column, 30*150 mm, 5 μm; Mobile Phase A: water (0.05% NH₃·H₂O), Mobile Phase B: CH₃CN; Flow rate: 60 mL/min; Gradient: 20% B to 50% B in 8 min, Wave Length: 254/220 nm. Finally, 4-{2-[(2R)-4-[(1R)-1-(3,4-difluorophenyl)ethyl]-2-(2-isopropoxyphenyl)piperazin-1-yl]-7-azaspiro[3.5]nonan-7-yl}-2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-N-[(3R)-5-nitro-3-(oxan-4-yl)-3,4-dihydro-2H-1,4-benzoxazin-7-ylsulfonyl]benzamide (assumed) was obtained as a yellow solid (6 mg, 21.1%). LC-MS (ESI, m/z) M+1: 1158. ¹H NMR (400 MHz, Methanol-d₄) δ 8.05-8.01 (m, 1H), 7.81-7.75 (m, 1H), 7.44-7.38 (m, 1H), 7.34-7.17 (m, 4H), 7.16-7.13 (m, 1H), 7.05-6.84 (m, 4H), 6.69-6.62 (m, 1H), 6.53-6.49 (m, 1H), 6.01-5.94 (m, 1H), 4.71-4.63 (m, 1H), 4.31-3.85 (m, 9H), 3.51-3.40 (m, 6H), 3.16-2.99 (m, 6H), 2.92-2.78 (m, 2H), 2.42-2.23 (m, 2H), 1.99-1.79 (m, 3H), 1.78-1.17 (m, 22H).

### Example 243 Preparation of 4-{2-[(2R)-4-[(1S)-1-(3,4-difluorophenyl)ethyl]-2-(2-isopropoxyphenyl)piperazin-1-yl]-7-azaspiro[3.5]nonan-7-yl}-2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-N-[(3R)-5-nitro-3-(oxan-4-yl)-3,4-dihydro-2H-1,4-benzoxazin-7-ylsulfonyl]benzamide (assumed)

Synthesis of 4-{2-[(2R)-4-[(1S)-1-(3,4-difluorophenyl)ethyl]-2-(2-isopropoxyphenyl)piperazin-1-yl]-7-azaspiro[3.5]nonan-7-yl}-2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-N-[(3R)-5-nitro-3-(oxan-4-yl)-3,4-dihydro-2H-1,4-benzoxazin-7-ylsulfonyl]benzamide (assumed): Into an 8-mL sealed tube, were placed 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-N-[(3R)-5-nitro-3-(oxan-4-yl)-3,4-dihydro-2H-1,4-benzoxazin-7-ylsulfonyl]-4-{2-oxo-7-azaspiro[3.5]nonan-7-yl}benzamide (20 mg, 0.03 mmol, 1.0 eq), (3R)-1-[(1S)-1-(3,4-difluorophenyl)ethyl]-3-(2-isopropoxyphenyl)piperazine (assumed) (11 mg, 0.03 mmol, 1.2 eq), ZnCl₂ (10 mg, 0.08 mmol, 3.0 eq), NaBH₃CN (5 mg, 0.08 mmol, 3.0 eq), MeOH (0.5 mL). The resulting solution was stirred for overnight at 70° C. in an oil bath. The crude product was purified by Flash-Prep-HPLC using the following conditions: Column: XBridge Shield RP18 OBD Column, 30*150 mm, 5 μm; Mobile Phase A: water (0.05% NH₃·H₂O), Mobile Phase B: CH₃CN; Flow rate: 60 mL/min; Gradient: 20% B to 50% B in 8 min, Wave Length: 254/220 nm. Finally, 4-{2-[(2R)-4-[(1S)-1-(3,4-difluorophenyl)ethyl]-2-(2-isopropoxyphenyl)piperazin-1-yl]-7-azaspiro[3.5]nonan-7-yl}-2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-N-[(3R)-5-nitro-3-(oxan-4-yl)-3,4-dihydro-2H-1,4-benzoxazin-7-ylsulfonyl]benzamide (assumed) was obtained as a yellow solid (6 mg, 21.1%). LC-MS (ESI, m/z) M+1: 1158. ¹H NMR (400 MHz, Methanol-d₄) δ 8.22-8.03 (m, 1H), 7.66-7.53 (m, 1H), 7.38-7.23 (m, 3H), 7.21-7.06 (m, 3H), 6.98-6.80 (m, 4H), 6.66-6.57 (m, 2H), 6.02-5.95 (m, 1H), 4.61-4.49 (m, 1H), 4.27-4.15 (m, 2H), 4.14-3.94 (m, 4H), 3.92-3.80 (m, 2H), 3.53-3.37 (m, 7H), 3.27-2.97 (m, 8H), 2.47-2.28 (m, 3H), 2.07-1.82 (m, 4H), 1.80-1.40 (m, 7H), 1.39-1.29 (m, 9H), 1.25-1.12 (m, 4H).

### Example 244 Preparation of 4-{2-[(2R)-4-(3,4-difluorobenzoyl)-2-(2-isopropoxyphenyl)piperazin-1-yl]-7-azaspiro[3.5]nonan-7-yl}-2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-N-[(3R)-5-nitro-3-(oxan-4-yl)-3,4-dihydro-2H-1,4-benzoxazin-7-ylsulfonyl]benzamide

Synthesis of (3R)-1-(3,4-difluorobenzoyl)-3-(2-isopropoxyphenyl)piperazine: Into an 8 mL vial were added 3,4-fifluorobenzoic acid (100 mg, 0.6 mmol, 1.0 eq), (2R)-2-(2-isopropoxyphenyl)piperazine (139 mg, 0.6 mmol, 1.0 eq), EDCl (133 mg, 0.7 mmol, 1.1 eq) and DCM (2 mL) at −20° C. The resulting mixture was stirred for 2 hours at −20° C. The reaction was quenched by the addition of water (3 mL) at −20° C. and extracted with CH₂Cl₂ (3×5 mL). The combined organic layers were washed with brine (5 mL), dried over anhydrous Na₂SO₄. After filtration, the filtrate was concentrated under vacuum. The residue was purified by reversed-phase flash chromatography using the following conditions: column, C18 silica gel; mobile phase, MeCN in water (0.1% NH₃·H₂O), 10% to 50% gradient in 10 min; detector, UV 254 nm. Finally, (3R)-1-(3,4-difluorobenzoyl)-3-(2-isopropoxyphenyl)piperazine was obtained as colorless oil (30 mg, 13.1%). LC-MS (ES, m/z) M+1: 361. ¹H NMR (300 MHz, Chloroform-d) δ 7.50 (d, J=7.3 Hz, 1H), 7.40-7.27 (m, 1H), 7.21 (d, J=5.7 Hz, 3H), 6.94 (d, J=7.3 Hz, 1H), 6.82 (s, 1H), 4.60 (d, J=52.2 Hz, 2H), 4.13 (s, 1H), 3.85 (s, 1H), 3.26 (s, 1H), 3.17-2.85 (m, 3H), 2.47 (s, 1H), 1.27 (s, 4H), 1.08 (s, 2H).

Synthesis of 4-{2-[(2R)-4-(3,4-difluorobenzoyl)-2-(2-isopropoxyphenyl)piperazin-1-yl]-7-azaspiro[3.5]nonan-7-yl}-2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-N-[(3R)-5-nitro-3-(oxan-4-yl)-3,4-dihydro-2H-1,4-benzoxazin-7-ylsulfonyl]benzamide: Into an 8 mL vial were added (3R)-1-(3,4-difluorobenzoyl)-3-(2-isopropoxyphenyl)piperazine (15 mg, 0.04 mmol, 1.0 eq), 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-N-[(3R)-5-nitro-3-(oxan-4-yl)-3,4-dihydro-2H-1,4-benzoxazin-7-ylsulfonyl]-4-{2-oxo-7-azaspiro[3.5]nonan-7-yl}benzamide (34 mg, 0.04 mmol, 1.0 eq), NaBH₃CN (13 mg, 0.2 mmol, 5.0 eq), ZnCl₂ (28 mg, 0.2 mmol, 5.0 eq) and MeOH (1 mL). The resulting mixture was stirred for overnight at 70° C. The reaction was quenched by the addition of water (5 mL) and extracted with CH₂Cl₂ (3×5 mL). The combined organic layers were washed with brine (5 mL), dried over anhydrous Na₂SO₄. After filtration, the filtrate was concentrated under vacuum. The residue was purified by reversed-phase flash chromatography using the following conditions: column, C18 silica gel; mobile phase, MeCN in water (0.1% NH₃·H₂O), 10% to 50% gradient in 10 min; detector, UV 254 nm. Finally 4-{2-[(2R)-4-(3,4-difluorobenzoyl)-2-(2-isopropoxyphenyl)piperazin-1-yl]-7-azaspiro[3.5]nonan-7-yl}-2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-N-[(3R)-5-nitro-3-(oxan-4-yl)-3,4-dihydro-2H-1,4-benzoxazin-7-ylsulfonyl]benzamide was obtained as a yellow solid (15 mg, 31.1%). LC-MS (ES, m/z) M+1: .1159. ¹H NMR (400 MHz, Chloroform-d) δ 12.62 (s, 1H), 8.45 (s, 1H), 8.38 (s, 1H), 8.32 (d, J=2.1 Hz, 1H), 8.13 (d, J=9.1 Hz, 1H), 7.47 (d, J=2.1 Hz, 1H), 7.44-7.32 (m, 2H), 7.24-7.14 (m, 3H), 7.13-7.07 (m, 1H), 6.86 (d, J=9.9 Hz, 2H), 6.70 (s, 1H), 6.45 (s, 1H), 6.14 (dd, J=3.7, 2.0 Hz, 1H), 4.71-4.35 (m, 5H), 4.29-3.91 (m, 6H), 3.44-3.31 (m, 7H), 3.26-2.85 (m, 8H), 1.76-1.68 (m, 7H), 1.63-1.55 (m, 6H), 1.56-1.38 (m, 5H), 1.25-1.18 (m, 4H).

### Example 245 Preparation of 4-(2-((R)-4-((3,4-difluorophenyl)sulfonyl)-2-(2-isopropoxyphenyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide

Synthesis of (R)-1-((3,4-difluorophenyl)sulfonyl)-3-(2-isopropoxyphenyl)piperazine: Into a 40 mL vial were added 3,4-difluorobenzenesulfonyl chloride (100 mg, 0.4 mmol, 0.8 eq), (2R)-2-(2-isopropoxyphenyl)piperazine (assumed) (130 mg, 0.5 mmol, 1.0 eq) and DCM (2 mL) at −10° C. To the above mixture was added DIEA (190 mg, 1.4 mmol, 2.5 eq) in portions over 1 minute at −10° C. The resulting mixture was stirred for 2 hours at −10° C. The reaction was quenched with water at −10° C. The resulting mixture was extracted with CH₂Cl₂ (1 mL). The combined organic layers were washed with brine (3×5 mL), dried over anhydrous Na₂SO₄. After filtration, the filtrate was concentrated under vacuum. The crude residue was purified by Prep-TLC (CH₂Cl₂/MeOH=10:1) to give (R)-1-((3,4-difluorophenyl)sulfonyl)-3-(2-isopropoxyphenyl)piperazine as a light yellow oil (148 mg, 63.9%). LC-MS (ES, m/z) M+1: 397. ¹H NMR (300 MHz, Chloroform-d) δ 7.68-7.50 (m, 2H), 7.35-7.31 (m, 2H), 7.24-7.23 (m, 1H), 6.91 (t, J=7.9 Hz, 2H), 5.32 (s, 1H), 4.63-4.62 (m, 1H), 4.26-4.24 (m, 1H), 3.91-3.88 (m, 1H), 3.73-3.71 (m, 1H), 3.24-3.01 (m, 2H), 2.52-2.50 (m, 1H), 2.24-2.22 (m, 1H), 1.39-1.36 (m, 6H).

Synthesis of 4-(2-((R)-4-((3,4-difluorophenyl)sulfonyl)-2-(2-isopropoxyphenyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide: Into an 8 mL vial were added (R)-1-((3,4-difluorophenyl)sulfonyl)-3-(2-isopropoxyphenyl)piperazine (8 mg, 0.1 mmol, 1.0 eq), 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-N-[(3R)-5-nitro-3-(oxan-4-yl)-3,4-dihydro-2H-1,4-benzoxazin-7-ylsulfonyl]-4-{2-oxo-7-azaspiro[3.5]nonan-7-yl}benzamide (17 mg, 0.1 mmol, 1.0 eq), NaBH₃CN (6 mg, 0.1 mmol, 5.0 eq), ZnCl₂ (14 mg, 0.1 mmol, 5.0 eq) and MeOH (1 mL). The resulting mixture was stirred for 4 hours at 70° C. The reaction was quenched with water and then extracted with CH₂Cl₂ (2 mL). The combined organic layers were washed with brine (3×5 mL), dried over anhydrous Na₂SO₄. After filtration, the filtrate was concentrated under vacuum. The crude residue was purified by Prep-TLC (CH₂Cl₂/MeOH=18:1) to give 4-(2-((R)-4-((3,4-difluorophenyl)sulfonyl)-2-(2-isopropoxyphenyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide as a yellow solid (14 mg, 53.2%). LC-MS (ES, m/z) M+1: 1195. ¹H NMR (300 MHz, Chloroform-d) δ 12.62 (s, 1H), 8.43-8.41 (m, 2H), 8.31 (d, J=2.1 Hz, 1H), 8.12-8.10 (m, 1H), 7.57-7.51 (m, 2H), 7.46 (d, J=2.1 Hz, 1H), 7.41-7.29 (m, 1H), 7.21-7.19 (m, 2H), 7.10-7.06 (m, 1H), 6.85-6.80 (m, 3H), 6.69 (s, 1H), 6.43 (d, J=2.4 Hz, 1H), 6.14-6.11 (m, 1H), 4.50-4.43 (m, 3H), 4.10-4.05 (m, 4H), 4.02-4.00 (m, 1H), 3.92-3.90 (m, 2H), 3.76-3.69 (m, 7H), 3.46-3.41 (m, 6H), 3.37-3.34 (m, 3H), 3.10-3.07 (m, 3H), 3.03-3.00 (m, 3H), 2.92-2.89 (m, 2H), 2.67-2.54 (m, 1H), 2.43-2.40 (m, 1H), 2.35-2.22 (m, 1H), 1.85-1.73 (m, 1H), 1.50-1.19 (m, 8H), 1.15-1.11 (m, 1H).

### Example 246 Preparation of 4-(2-((R)-4-((R)-1-(3,4-difluorophenyl)propyl)-2-(2-isopropoxyphenyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide (assumed)

Synthesis of 1-(3,4-difluorophenyl)propan-1-ol: A solution of 3,4-difluorobenzaldehyde (1.0 g, 7.1 mmol, 1.0 eq) in THE (10 mL) for 15 minutes at −20° C. under nitrogen atmosphere followed by the addition of EtMgBr (4.0 mL, 31.8 mmol, 4.5 eq) in portions at −20° C. The resulting mixture was stirred for 1 hour at −20° C. The reaction was quenched with sat. NH₄Cl (aq.) at 0° C. The resulting mixture was extracted with EtOAc (10 mL). The combined organic layers were washed with brine (3×5 mL), dried over anhydrous Na₂SO₄. After filtration, the filtrate was concentrated under vacuum. The crude residue was purified by silica gel column chromatography, eluted with ethyl acetate/petroleum ether=1:5 to give 1-(3,4-difluorophenyl)propan-1-ol as a colorless oil (700 mg, 57.7%). ¹H NMR (300 MHz, Chloroform-d) δ 7.24-7.00 (m, 3H), 4.59 (t, J=6.5 Hz, 1H), 1.86-1.68 (m, 2H), 0.92 (t, J=7.4 Hz, 3H).

Synthesis of 4-(1-chloropropyl)-1,2-difluorobenzene: Into a 40 mL vial were added 1-(3,4-difluorophenyl)propan-1-ol (700 mg, 4.1 mmol, 1.0 eq) and CHCl₃ (3 mL, 2.1 mmol, 0.5 eq) at 0° C. To the above mixture was added SOCl₂ (6.0 mL, 75.8 mmol, 18.6 eq) in portions over 5 minutes at 0° C. The resulting mixture was stirred for 4 hours at 25° C. The reaction was quenched with sat. NH₄Cl (aq.) at 25° C. The filtrate was concentrated under vacuum. The resulting mixture was diluted with CH₂Cl₂ (2 mL), was washed with brine (3×5 mL), dried over anhydrous Na₂SO₄. After filtration, the filtrate was concentrated under vacuum. To give 4-(1-chloropropyl)-1,2-difluorobenzene as a light yellow oil (389 mg, 50.1%). ¹H NMR (300 MHz, Chloroform-d) δ 7.25-7.00 (m, 3H), 4.74 (t, J=7.2 Hz, 1H), 2.18-1.96 (m, 2H), 1.01 (t, J=7.3 Hz, 3H).

Synthesis of (R)-1-((R)-1-(3,4-difluorophenyl)propyl)-3-(2-isopropoxyphenyl)piperazine (assumed) and (R)-1-((S)-1-(3,4-difluorophenyl)propyl)-3-(2-isopropoxyphenyl)piperazine (assumed): Into an 8 mL vial were added 4-(1-chloropropyl)-1,2-difluorobenzene (86 mg, 0.4 mmol, 1.0 eq), (2R)-2-(2-isopropoxyphenyl)piperazine (assumed) (99 mg, 0.4 mmol, 1.0 eq), K₂CO₃ (187 mg, 1.3 mmol, 3.0 eq) and MeCN (2 mL). The resulting mixture was stirred for 4 hours at 80° C. The reaction was quenched with water at 25° C. The resulting mixture was extracted with CH₂Cl₂ (3 mL). The combined organic layers were washed with brine (5 mL), dried over anhydrous Na₂SO₄. After filtration, the filtrate was concentrated under vacuum. The crude residue was purified by Prep-TLC (ethyl acetate) to give (R)-1-((R)-1-(3,4-difluorophenyl)propyl)-3-(2-isopropoxyphenyl)piperazine (assumed) as a light yellow oil (30 mg, 17.7%) and (R)-1-((S)-1-(3,4-difluorophenyl)propyl)-3-(2-isopropoxyphenyl)piperazine (assumed) as a light yellow solid (20 mg, 11.8%).

Synthesis of 4-(2-((R)-4-((R)-1-(3,4-difluorophenyl)propyl)-2-(2-isopropoxyphenyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide (assumed): Into an 8 mL vial were added (R)-1-((R)-1-(3,4-difluorophenyl)propyl)-3-(2-isopropoxyphenyl)piperazine (assumed) (8 mg, 0.1 mmol, 1.0 eq), 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-N-[(3R)-5-nitro-3-(oxan-4-yl)-3,4-dihydro-2H-1,4-benzoxazin-7-ylsulfonyl]-4-{2-oxo-7-azaspiro[3.5]nonan-7-yl}benzamide (18 mg, 0.1 mmol, 1.0 eq), NaBH₃CN (6 mg, 0.1 mmol, 5.0 eq), ZnCl₂ (15 mg, 0.1 mmol, 5.0 eq) and MeOH (1 mL). The resulting mixture was stirred for 4 hours at 70° C. The reaction was quenched with water, and extracted with CH₂Cl₂ (3 mL). The combined organic layers were washed with brine (5 mL), dried over anhydrous Na₂SO₄. After filtration, the filtrate was concentrated under vacuum. The crude residue was purified by Prep-TLC (CH₂Cl₂/MeOH=18:1) to give 4-(2-((R)-4-((R)-1-(3,4-difluorophenyl)propyl)-2-(2-isopropoxyphenyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide (assumed) as a yellow solid (14 mg, 53.8%). LC-MS (ES, m/z) M+1: 1173. ¹H NMR (300 MHz, Chloroform-d) δ 12.64 (s, 1H), 8.46 (s, 2H), 8.31 (d, J=2.1 Hz, 1H), 8.12-8.11 (m, 1H), 7.46 (d, J=2.1 Hz, 1H), 7.39 (d, J=7.6 Hz, 1H), 7.23-7.08 (m, 4H), 6.96 (s, 1H), 6.86-6.83 (m, 3H), 6.69 (s, 1H), 6.43 (d, J=2.5 Hz, 1H), 6.13-3.11 (m, 1H), 4.51-4.46 (m, 4H), 4.16-3.98 (m, 5H), 3.98-3.84 (m, 2H), 3.50-3.47 (m, 2H), 3.41-3.36 (m, 5H), 3.11-3.09 (m, 3H), 3.04-3.00 (m, 2H), 2.93-2.90 (m, 1H), 2.84-2.80 (m, 2H), 2.72-2.68 (m, 1H), 2.20-2.00 (m, 4H), 1.79-1.75 (m, 4H), 1.65-1.63 (m, 7H), 1.36-1.33 (m, 9H), 1.26-1.22 (m, 1H), 1.19-1.16 (m, 1H).

### Example 247 Preparation of 4-(2-((R)-4-((S)-1-(3,4-difluorophenyl)propyl)-2-(2-isopropoxyphenyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide (assumed)

Synthesis of 4-(2-((R)-4-((S)-1-(3,4-difluorophenyl)propyl)-2-(2-isopropoxyphenyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide (assumed): Into an 8 mL vial were added (R)-1-((S)-1-(3,4-difluorophenyl)propyl)-3-(2-isopropoxyphenyl)piperazine (assumed) (20 mg, 0.3 mmol, 2.0 eq), 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-N-[(3R)-5-nitro-3-(oxan-4-yl)-3,4-dihydro-2H-1,4-benzoxazin-7-ylsulfonyl]-4-{2-oxo-7-azaspiro[3.5]nonan-7-yl}benzamide (21 mg, 0.1 mmol, 1.0 eq), NaBH₃CN (8 mg, 0.1 mmol, 5.0 eq), ZnCl₂ (18 mg, 0.1 mmol, 5.0 eq) and MeOH (1 mL). The resulting mixture was stirred for 4 hours at 70° C. The reaction was quenched with water and then extracted with CH₂Cl₂ (3 mL). The combined organic layers were washed with brine (5 mL), dried over anhydrous Na₂SO₄. After filtration, the filtrate was concentrated under vacuum. The crude residue was purified by Prep-TLC (CH₂Cl₂/MeOH=18:1) to give 4-(2-((R)-4-((S)-1-(3,4-difluorophenyl)propyl)-2-(2-isopropoxyphenyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide (assumed) as a yellow solid (10 mg, 31.9%). LC-MS (ES, m/z) M+1: 1173. ¹H NMR (300 MHz, Chloroform-d) δ 12.64 (s, 1H), 8.45 (s, 1H), 8.31 (d, J=2.2 Hz, 2H), 8.12-8.11 (m, 2H), 7.47 (d, J=2.1 Hz, 1H), 7.34-7.30 (m, 2H), 7.13 (s, 1H), 7.09 (s, 2H), 7.02 (s, 1H), 6.91 (s, 1H), 6.82-6.78 (m, 3H), 6.69-6.63 (m, 1H), 6.43 (s, 1H), 6.14 (s, 1H), 4.60-4.41 (m, 4H), 4.16-3.93 (m, 6H), 3.77-3.73 (m, 1H), 3.55-3.34 (m, 7H), 3.29-3.26 (m, 1H), 3.14-3.05 (m, 4H), 2.96-2.93 (m, 2H), 2.50-2.48 (m, 1H), 2.29-2.28 (m, 2H), 2.10-2.06 (m, 1H), 1.93-1.88 (m, 3H), 1.81-1.80 (m, 2H), 1.79-1.66 (m, 5H), 1.43-1.36 (m, 3H), 1.32-1.30 (m, 4H), 1.19-1.16 (m, 3H), 0.77-0.62 (m, 2H).

### Example 248 Preparation of 4-{2-[(2R)-4-[(4S)-7,8-difluoro-3,4-dihydro-1H-2-benzopyran-4-yl]-2-(2-isopropoxyphenyl)piperazin-1-yl]-7-azaspiro[3.5]nonan-7-yl}-2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-N-[(3R)-5-nitro-3-(oxan-4-yl)-3,4-dihydro-2H-1,4-benzoxazin-7-ylsulfonyl]benzamide (assumed)

Synthesis of tert-butyl 2-[(6-bromo-2,3-difluorophenyl)methoxy]acetate: To a stirred solution of (6-bromo-2,3-difluorophenyl)methanol (4.3 g, 19.3 mmol, 1.0 eq) and NaH (0.9 g, 38.6 mmol, 2.0 eq) in THF(50 mL) for 1 hour under 0° C. was added tert-butyl 2-bromoacetate (5.6 g, 28.9 mmol, 1.5 eq) dropwise at 0° C. under nitrogen atmosphere. The resulting mixture was stirred for 4 hours at 25° C. under nitrogen atmosphere. The reaction was quenched by the addition of water (20 mL) and extracted with EtOAc (3×50 mL). The combined organic layers were washed with brine (50 mL), dried over anhydrous Na₂SO₄. After filtration, the filtrate was concentrated under vacuum. The residue was applied onto a silica gel column and eluted with ethyl acetate/petroleum ether=1:3 to give tert-butyl 2-[(6-bromo-2,3-difluorophenyl)methoxy]acetate as an off-white solid (6.5 g, 99.9%). ¹H NMR (300 MHz, Chloroform-d) δ 7.36 (dq, J=6.8, 2.2 Hz, 1H), 7.08 (ddd, J=9.6, 8.9, 8.1 Hz, 1H), 4.84 (d, J=2.7 Hz, 2H), 4.05 (s, 2H), 1.50 (s, 9H).

Synthesis of [(6-bromo-2,3-difluorophenyl)methoxy]acetic acid: Into a 100 mL round-bottom flask were added tert-butyl 2-[(6-bromo-2,3-difluorophenyl)methoxy]acetate (6.5 g, 19.3 mmol, 1.0 eq), TFA (10 mL, 134.6 mmol, 7.0 eq) and DCM (30 mL). The resulting mixture was stirred for 4 hours at 25° C. The resulting mixture was concentrated under vacuum to give [(6-bromo-2,3-difluorophenyl)methoxy]acetic acid (4.1 g, 75.7%) as a colorless solid without further purification. ¹H NMR (300 MHz, DMSO-d₆) δ 12.78 (bs, 1H), 7.72-7.35 (m, 2H), 4.71 (s, 2H), 4.08 (s, 2H).

Synthesis of 2-[(6-bromo-2,3-difluorophenyl)methoxy]-N-methoxy-N-methylacetamide: Into a 100 mL round-bottom flask were added [(6-bromo-2,3-difluorophenyl)methoxy]acetic acid (4.1 g, 14.6 mmol, 1.0 eq), N,O-dimethylhydroxylamine (0.9 g, 14.6 mmol, 1.0 eq), HATU (11.1 g, 29.2 mmol, 2.0 eq), DIEA (5.7 g, 43.8 mmol, 3.0 eq) and THE (40 mL). The resulting mixture was stirred for 2 hours at 25° C. The reaction was quenched with water (20 mL), and then extracted with EtOAc (3×30 mL). The combined organic layers were washed with brine (30 mL), dried over anhydrous Na₂SO₄. After filtration, the filtrate was concentrated under vacuum. The residue was applied onto a silica gel column and eluted with ethyl acetate/petroleum ether=1:3 to give 2-[(6-bromo-2,3-difluorophenyl)methoxy]-N-methoxy-N-methylacetamide as a white solid (3.6 g, 76.1%). ¹H NMR (400 MHz, Chloroform-d) δ 7.37 (ddd, J=8.9, 4.4, 2.1 Hz, 1H), 7.08 (td, J=9.3, 8.1 Hz, 1H), 4.87 (d, J=2.7 Hz, 2H), 4.36 (s, 2H), 3.70 (s, 3H), 3.23 (s, 3H).

Synthesis of 7,8-difluoro-1,3-dihydro-2-benzopyran-4-one: A solution of 2-[(6-bromo-2,3-difluorophenyl)methoxy]-N-methoxy-N-methylacetamide (3.6 g, 11.1 mmol, 1.0 eq) in THE (40 mL) was treated with n-BuLi (1.1 g, 16.7 mmol, 1.5 eq) dropwise for 20 min at −78° C. under nitrogen atmosphere. The resulting mixture was stirred for 3 hours at −78° C. under nitrogen atmosphere. The reaction was quenched by the addition of sat. NH₄Cl (aq.) (20 mL) at −78° C., and then extracted with EtOAc (3×50 mL). The combined organic layers were washed with brine (50 mL), dried over anhydrous Na₂SO₄. After filtration, the filtrate was concentrated under vacuum. The residue was applied onto a silica gel column and eluted with ethyl acetate/petroleum ether=1:5 7,8-difluoro-1,3-dihydro-2-benzopyran-4-one as a white solid (1.9 g, 90.5%). ¹H NMR (400 MHz, Chloroform-d) δ 7.89 (ddd, J=8.7, 4.8, 1.7 Hz, 1H), 7.30-7.18 (m, 1H), 5.01 (s, 2H), 4.37 (s, 2H).

Synthesis of 7,8-difluoro-3,4-dihydro-1H-2-benzopyran-4-ol: A solution of 7,8-difluoro-1,3-dihydro-2-benzopyran-4-one (700 mg, 3.8 mmol, 1.0 eq) in MeOH (8 mL) was treated with NaBH₄ (288 mg, 7.6 mmol, 2.0 eq) for 10 min at 0° C. The resulting mixture was stirred for 1 hour at 25° C. The reaction was quenched by the addition of water (5 mL) at 0° C., and then extracted with CH₂Cl₂ (3×10 mL). The combined organic layers were washed with brine (10 mL), dried over anhydrous Na₂SO₄. After filtration, the filtrate was concentrated under vacuum to afford 7,8-difluoro-3,4-dihydro-1H-2-benzopyran-4-ol as an off-white solid (650 mg, 91.8%). ¹H NMR (300 MHz, Chloroform-d) δ 7.22 (ddd, J=8.8, 4.6, 1.5 Hz, 1H), 7.18-7.04 (m, 1H), 4.92 (d, J=15.9 Hz, 1H), 4.65 (d, J=15.9 Hz, 1H), 4.53 (dt, J=9.4, 2.9 Hz, 1H), 4.08 (dd, J=12.0, 2.9 Hz, 1H), 3.83 (dd, J=12.1, 2.7 Hz, 1H), 2.53 (d, J=9.3 Hz, 1H).

Synthesis of 4-chloro-7,8-difluoro-3,4-dihydro-1H-2-benzopyran: Into a 50 mL round-bottom flask were added 7,8-difluoro-3,4-dihydro-1H-2-benzopyran-4-ol (650 mg, 3.5 mmol, 1.0 eq), CHCl₃ (2 mL) and thionyl chloride (8 mL) at 0° C. The resulting mixture was stirred for 4 hours at 25° C. The resulting mixture was concentrated under vacuum. The resulting mixture was diluted with water (10 mL). The resulting mixture was extracted with CH₂Cl₂ (3×10 mL). The combined organic layers were washed with brine (10 mL), dried over anhydrous Na₂SO₄. After filtration, the filtrate was concentrated under vacuum to give 4-chloro-7,8-difluoro-3,4-dihydro-1H-2-benzopyran as an off-white solid (680 mg, 95.2%). ¹H NMR (300 MHz, Chloroform-d) δ 7.33-7.22 (m, 1H), 7.22-7.05 (m, 1H), 5.11-4.95 (m, 2H), 4.81 (d, J=16.0 Hz, 1H), 4.22-4.03 (m, 2H).

Synthesis of (3R)-1-[(4S)-7,8-difluoro-3,4-dihydro-1H-2-benzopyran-4-yl]-3-(2-isopropoxyphenyl)piperazine (assumed) and (3R)-1-[(4R)-7,8-difluoro-3,4-dihydro-1H-2-benzopyran-4-yl]-3-(2-isopropoxyphenyl)piperazine (assumed): Into a 40 mL vial were added 4-chloro-7,8-difluoro-3,4-dihydro-1H-2-benzopyran (300 mg, 1.5 mmol, 1.0 eq), (2R)-2-(2-isopropoxyphenyl)piperazine (355 mg, 1.6 mmol, 1.1 eq), K₂CO₃ (608 mg, 4.4 mmol, 3.0 eq) and CH₃CN (6 mL). The resulting mixture was stirred for 5 hours at 80° C. The reaction was quenched by the addition of water (10 mL), and extracted with CH₂Cl₂ (3×10 mL). The combined organic layers were washed with brine (10 mL), dried over anhydrous Na₂SO₄. After filtration, the filtrate was concentrated under vacuum. The residue was purified by Prep-TLC (EA 100%) to give (3R)-1-[(4S)-7,8-difluoro-3,4-dihydro-1H-2-benzopyran-4-yl]-3-(2-isopropoxyphenyl)piperazine (assumed) (42 mg, 7.4%) and (3R)-1-[(4R)-7,8-difluoro-3,4-dihydro-1H-2-benzopyran-4-yl]-3-(2-isopropoxyphenyl)piperazine (assumed) as a colorless solid (36 mg, 6.3%). ¹H NMR_A (300 MHz, Chloroform-d) δ 7.52 (d, J=7.3 Hz, 1H), 7.35 (t, J=6.8 Hz, 1H), 7.24 (t, J=7.8 Hz, 1H), 7.10 (q, J=8.9 Hz, 1H), 6.97-6.85 (m, 2H), 4.89 (d, J=15.7 Hz, 1H), 4.63 (td, J=12.2, 11.2, 5.1 Hz, 2H), 4.47-4.35 (m, 1H), 4.24 (dd, J=12.1, 3.8 Hz, 1H), 3.78 (dd, J=12.0, 4.3 Hz, 1H), 3.68 (s, 1H), 3.11 (t, J=9.5 Hz, 2H), 3.01-2.84 (m, 1H), 2.84-2.58 (m, 3H), 1.40 (dd, J=6.0, 5.0 Hz, 6H). ¹H NMR_B (300 MHz, Chloroform-d) δ 7.45 (dd, J=7.7, 1.8 Hz, 2H), 7.16 (ddd, J=9.8, 5.9, 2.1 Hz, 1H), 7.13-7.02 (m, 1H), 6.89 (dd, J=8.0, 6.9 Hz, 1H), 6.79 (d, J=8.2 Hz, 1H), 4.82 (d, J=15.7 Hz, 1H), 4.63 (d, J=15.7 Hz, 1H), 4.49 (hept, J=6.3 Hz, 1H), 4.20 (dd, J=11.9, 4.3 Hz, 1H), 4.06 (dd, J=9.5, 2.7 Hz, 1H), 3.88-3.71 (m, 2H), 3.28-3.06 (m, 2H), 3.00-2.72 (m, 3H), 2.40-2.20 (m, 1H), 1.30-1.27 (m, 3H), 1.09 (d, J=6.0 Hz, 3H).

Synthesis of 4-{2-[(2R)-4-[(4S)-7,8-difluoro-3,4-dihydro-1H-2-benzopyran-4-yl]-2-(2-isopropoxyphenyl)piperazin-1-yl]-7-azaspiro[3.5]nonan-7-yl}-2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-N-[(3R)-5-nitro-3-(oxan-4-yl)-3,4-dihydro-2H-1,4-benzoxazin-7-ylsulfonyl]benzamide (assumed): Into an 8 mL vial were added (3R)-1-[(4S)-7,8-difluoro-3,4-dihydro-1H-2-benzopyran-4-yl]-3-(2-isopropoxyphenyl)piperazine (20 mg, 0.05 mmol, 1.0 eq), 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-N-[(3R)-5-nitro-3-(oxan-4-yl)-3,4-dihydro-2H-1,4-benzoxazin-7-ylsulfonyl]-4-{2-oxo-7-azaspiro[3.5]nonan-7-yl}benzamide (42 mg, 0.05 mmol, 1.0 eq), NaBH₃CN (16 mg, 0.2 mmol, 5.0 eq), ZnCl₂ (35 mg, 0.2 mmol, 5.0 eq) and MeOH (1 mL). The resulting mixture was stirred for 4 hours at 70° C. The reaction was quenched by the addition of water (3 mL). The resulting mixture was extracted with CH₂Cl₂ (3×5 mL). The combined organic layers were washed with brine (5 mL), dried over anhydrous Na₂SO₄. After filtration, the filtrate was concentrated under vacuum. The residue was purified by reversed-phase flash chromatography using the following conditions: column, C18 silica gel; mobile phase, MeCN in water (0.1% FA), 10% to 50% gradient in 10 min; detector, UV 254 nm. Finally 4-{2-[(2R)-4-[(4S)-7,8-difluoro-3,4-dihydro-1H-2-benzopyran-4-yl]-2-(2-isopropoxyphenyl)piperazin-1-yl]-7-azaspiro[3.5]nonan-7-yl}-2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-N-[(3R)-5-nitro-3-(oxan-4-yl)-3,4-dihydro-2H-1,4-benzoxazin-7-ylsulfonyl]benzamide (assumed) was obtained as a yellow solid (15 mg, 24.5%). LC-MS (ES, m/z) M+1: 1187. ¹H NMR (400 MHz, Chloroform-d) δ 12.62 (s, 1H), 8.43 (d, J=12.7 Hz, 2H), 8.31 (d, J=2.1 Hz, 1H), 8.13 (d, J=9.2 Hz, 1H), 7.46 (d, J=2.2 Hz, 2H), 7.09 (s, 2H), 6.97 (s, 2H), 6.86 (t, J=10.9 Hz, 1H), 6.69 (s, 1H), 6.43 (s, 1H), 6.13 (t, J=2.8 Hz, 1H), 4.89-4.85 (m, 1H), 4.63-4.58 (m, 2H), 4.58-4.48 (m, 4H), 4.45-4.41 (m, 3H), 4.26-4.21 (m, 2H), 4.15-4.03 (m, 4H), 3.98-3.95 (m, 1H), 3.80-3.73 (m, 8H), 3.68-3.65 (m, 1H), 3.45-3.38 (m, 6H), 3.07-2.98 (m, 4H), 2.78 (s, 3H), 1.85-1.78 (m, 3H), 1.56-1.43 (m, 4H), 1.40 (d, J=6.0 Hz, 6H), 1.12-1-0.90 (m, 1H).

### Example 249 Preparation of 4-{2-[(2R)-4-[(4R)-7,8-difluoro-3,4-dihydro-1H-2-benzopyran-4-yl]-2-(2-isopropoxyphenyl)piperazin-1-yl]-7-azaspiro[3.5]nonan-7-yl}-2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-N-[(3R)-5-nitro-3-(oxan-4-yl)-3,4-dihydro-2H-1,4-benzoxazin-7-ylsulfonyl]benzamide (assumed)

Synthesis of 4-{2-[(2R)-4-[(4R)-7,8-difluoro-3,4-dihydro-1H-2-benzopyran-4-yl]-2-(2-isopropoxyphenyl)piperazin-1-yl]-7-azaspiro[3.5]nonan-7-yl}-2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-N-[(3R)-5-nitro-3-(oxan-4-yl)-3,4-dihydro-2H-1,4-benzoxazin-7-ylsulfonyl]benzamide (assumed): Into an 8 mL vial were added (3R)-1-[(4R)-7,8-difluoro-3,4-dihydro-1H-2-benzopyran-4-yl]-3-(2-isopropoxyphenyl)piperazine (20 mg, 0.05 mmol, 1.0 eq), 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-N-[(3R)-5-nitro-3-(oxan-4-yl)-3,4-dihydro-2H-1,4-benzoxazin-7-ylsulfonyl]-4-{2-oxo-7-azaspiro[3.5]nonan-7-yl}benzamide (42 mg, 0.05 mmol, 1.0 eq), NaBH₃CN (16 mg, 0.2 mmol, 5.0 eq), ZnCl₂ (35 mg, 0.2 mmol, 5.0 eq) and MeOH (0.5 mL). The resulting mixture was stirred for 4 hours at 70° C. The reaction was quenched by the addition of water (3 mL). The resulting mixture was extracted with CH₂Cl₂ (3×5 mL). The combined organic layers were washed with brine (5 mL), dried over anhydrous Na₂SO₄. After filtration, the filtrate was concentrated under vacuum. The residue was purified by reversed-phase flash chromatography using the following conditions: column, C18 silica gel; mobile phase, MeCN in water (0.1% FA), 10% to 50% gradient in 10 min; detector, UV 254 nm. Finally, 4-{2-[(2R)-4-[(4R)-7,8-difluoro-3,4-dihydro-1H-2-benzopyran-4-yl]-2-(2-isopropoxyphenyl)piperazin-1-yl]-7-azaspiro[3.5]nonan-7-yl}-2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-N-[(3R)-5-nitro-3-(oxan-4-yl)-3,4-dihydro-2H-1,4-benzoxazin-7-ylsulfonyl]benzamide was obtained as a yellow solid (16 mg, 26.2%). LC-MS (ES, m/z) M+1: 1187. ¹H NMR (400 MHz, Chloroform-d) δ 12.62 (s, 1H), 8.45 (d, J=3.0 Hz, 1H), 8.40 (s, 1H), 8.31 (d, J=2.1 Hz, 1H), 8.13 (d, J=9.1 Hz, 1H), 7.46 (d, J=2.2 Hz, 1H), 7.18 (s, 1H), 7.13-7.07 (m, 1H), 7.03 (d, J=17.0 Hz, 1H), 6.88-6.81 (m, 1H), 6.82 (s, 2H), 6.69 (s, 1H), 6.44 (d, J=2.5 Hz, 1H), 6.14 (dd, J=3.5, 2.0 Hz, 1H), 4.81-4.77 (m, 1H), 4.65-4.41 (m, 4H), 4.21-4.18 (m, 2H), 4.13-4.11 (m, 2H), 4.10-4.03 (m, 2H), 3.98-3.85 (m, 1H), 3.79-3.71 (m, 4H), 3.45-3.36 (m, 7H), 3.13-3.04 (m, 5H), 3.00 (s, 3H), 2.47 (s, 3H), 1.88-1.78 (m, 2H), 1.52-1.48 (m, 6H), 1.33-1.28 (m, 7H), 1.15 (d, J=6.0 Hz, 4H).

### Example 250 Preparation of 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-4-{2-[(2R)-2-(2-isopropoxyphenyl)-4-[(4S)-8-methoxy-7-(morpholin-4-yl)-1H,3H,4H-pyrano[4,3-c]pyridin-4-yl]piperazin-1-yl]-7-azaspiro[3.5]nonan-7-yl}-N-[(3R)-5-nitro-3-(oxan-4-yl)-3,4-dihydro-2H-1,4-benzoxazin-7-ylsulfonyl]benzamide (assumed)

Synthesis of 5-bromo-2-fluoro-3-(methoxymethoxy)pyridine: Into a 1000 mL 3-necked round-bottom flask, were placed 5-bromo-2-fluoropyridin-3-ol (30.0 g, 156.3 mmol, 1.0 eq), THE (300 mL), DBU (71.4 g, 468.8 mmol, 3.0 eq). This was followed by the addition of bromo(methoxy)methane (48.8 g, 390.7 mmol, 2.5 eq) at 0° C. The resulting mixture was stirred for 1 hour at 25° C. The resulting mixture was then quenched by the addition of water (200 mL), and then extracted with ethyl acetate (2×200 mL). The combined organic layers was washed with brine (2×200 mL). The mixture was dried over anhydrous Na₂SO₄. After filtration, the filtrate was concentrated under vacuum. The crude residue was purified onto a silica gel column and eluted with ethyl acetate/petroleum ether=3:1 to give 5-bromo-2-fluoro-3-(methoxymethoxy)pyridine as an off-white solid (33.0 g, 89.5%). ¹H NMR (300 MHz, DMSO-d₆) δ 7.96 (dq, J=4.5, 2.1 Hz, 2H), 5.37 (s, 2H), 3.43 (s, 3H).

Synthesis of 5-bromo-2-fluoro-3-(methoxymethoxy)pyridine-4-carbaldehyde: Into a 1000 mL 3-necked round-bottom flask under nitrogen atmosphere, were placed LDA (18.0 g, 167.8 mmol, 1.2 eq), which was followed by the addition of 5-bromo-2-fluoro-3-(methoxymethoxy)pyridine (33.0 g, 139.8 mmol, 1.0 eq) in THE (330 mL) at −78° C. The resulting mixture was stirred for 3 hours at −78° C. To the above mixture was added methyl formate (21.0 g, 349.5 mmol, 2.5 eq) at −78° C. The resulting mixture was stirred for additional 3 hours at −78° C. The resulting mixture was then quenched by the addition of NaHCO₃(aq.) (100 mL). The mixture was extracted with ethyl acetate (2×100 mL) and washed with brine (2×100 mL). The mixture was dried over anhydrous Na₂SO₄. After filtration, the filtrate was concentrated under vacuum. The crude residue was purified onto a silica gel column and eluted with ethyl acetate/petroleum ether=1:5 to give 5-bromo-2-fluoro-3-(methoxymethoxy)pyridine-4-carbaldehyde as a light yellow oil (20.0 g, 54.2%). ¹H NMR (300 MHz, DMSO-d₆) δ 10.21 (d, J=0.9 Hz, 1H), 8.31 (d, J=1.8 Hz, 1H), 5.26 (d, J=1.2 Hz, 2H), 3.49 (s, 3H).

Synthesis of [5-bromo-2-fluoro-3-(methoxymethoxy)pyridin-4-yl]methanol: Into a 500 mL 3-necked round-bottom flask, were placed 5-bromo-2-fluoro-3-(methoxymethoxy)pyridine-4-carbaldehyde (20.0 g, 75.7 mmol, 1.0 eq), MeOH (200 mL). This was followed by the addition of NaBH₄ (4.3 g, 113.6 mmol, 1.5 eq) at 0° C. The resulting mixture was stirred for 2 hours at 25° C. The resulting mixture was then quenched by the addition of water (100 mL). The mixture was extracted with ethyl acetate (2×100 mL) and washed with brine (2×100 mL). The mixture was dried over anhydrous Na₂SO₄. After filtration, the filtrate was concentrated under vacuum. The crude residue was purified onto a silica gel column and eluted with ethyl acetate/petroleum ether=1:3 to give [5-bromo-2-fluoro-3-(methoxymethoxy)pyridin-4-yl]methanol as a light yellow oil (18.0 g, 89.3%). ¹H NMR (300 MHz, DMSO-d₆) δ 8.18 (d, J=1.5 Hz, 1H), 5.41 (t, J=5.4 Hz, 1H), 5.21 (d, J=0.9 Hz, 2H), 4.61 (d, J=5.4 Hz, 2H), 3.52 (s, 3H).

Synthesis of 5-bromo-2-fluoro-4-(hydroxymethyl)pyridin-3-ol: Into a 500 mL 3-necked round-bottom flask, were placed [5-bromo-2-fluoro-3-(methoxymethoxy)pyridin-4-yl]methanol (18.0 g, 67.7 mmol, 1.0 eq), DCM (90 mL), HCl (gas) in 1,4-dioxane (90 mL). The reaction mixture was stirred for 1 hour at 25° C. The resulting mixture was concentrated under vacuum to give 5-bromo-2-fluoro-4-(hydroxymethyl)pyridin-3-ol as an off-white solid (13.0 g, 86.6%). ¹H NMR (300 MHz, DMSO-d₆) δ 11.0-10.62 (m, 1H), 7.88-7.77 (m, 1H), 4.82-4.60 (m, 1H), 3.57 (s, 2H).

Synthesis of (5-bromo-2-fluoro-3-methoxypyridin-4-yl)methanol: Into a 500 mL 3-necked round-bottom flask, were placed 5-bromo-2-fluoro-4-(hydroxymethyl)pyridin-3-ol (13.0 g, 58.6 mmol, 1.0 eq), DMF (150 mL), K₂CO₃ (24.3 g, 175.7 mmol, 3.0 eq). This was followed by the addition of CH₃I (10.0 g, 70.3 mmol, 1.2 eq) at 0° C. The resulting mixture was stirred for 2 hours at 25° C. The resulting mixture was then quenched by the addition of water (100 mL). The mixture was extracted with ethyl acetate (2×100 mL) and washed with brine (2×100 mL). The mixture was dried over anhydrous Na₂SO₄. After filtration, the filtrate was concentrated under vacuum. The crude residue was purified onto a silica gel column and eluted with ethyl acetate/petroleum ether=1:3 to give (5-bromo-2-fluoro-3-methoxypyridin-4-yl)methanol as a light yellow oil (7.5 g, 54.3%). ¹H NMR (300 MHz, DMSO-d₆) δ 8.14 (d, J=1.8 Hz, 1H), 5.39 (t, J=5.4 Hz, 1H), 4.58 (d, J=5.4 Hz, 2H), 3.92 (d, J=1.8 Hz, 3H).

Synthesis of ethyl 2-[(5-bromo-2-fluoro-3-methoxypyridin-4-yl)methoxy]acetate: Into a 250 mL 3-necked round-bottom flask, were placed (5-bromo-2-fluoro-3-methoxypyridin-4-yl)methanol (7.5 g, 31.8 mmol, 1.0 eq), DCM (80 mL), 1,1,1-tris(acetyloxy)dirhodium-1-yl acetate (1.4 g, 3.2 mmol, 0.1 eq). This was followed by the addition of N-(formylimino)propanamide (3.6 g, 31.8 mmol, 1.0 eq) at 0° C. The resulting mixture was stirred for 4 hours at 25° C. The resulting mixture was then quenched by the addition of water (80 mL). The mixture was extracted with dichloromethane (2×80 mL) and washed with brine (2×80 mL). The mixture was dried over anhydrous Na₂SO₄. After filtration, the filtrate was concentrated under vacuum. The crude residue was purified onto a silica gel column and eluted with ethyl acetate/petroleum ether=1:5 to give ethyl 2-[(5-bromo-2-fluoro-3-methoxypyridin-4-yl)methoxy]acetate as a white solid (2.3 g, 22.5%). ¹H NMR (300 MHz, DMSO-d₆) δ 8.18 (d, J=1.8 Hz, 1H), 4.70 (s, 2H), 4.19 (s, 2H), 4.16-4.09 (m, 2H), 3.93 (d, J=2.1 Hz, 3H), 1.21 (t, J=7.2 Hz, 3H).

Synthesis of [(5-bromo-2-fluoro-3-methoxypyridin-4-yl)methoxy]acetic acid: Into a 100 mL round-bottom flask, were placed ethyl 2-[(5-bromo-2-fluoro-3-methoxypyridin-4-yl)methoxy]acetate (2.3 g, 7.1 mmol, 1.0 eq), LiOH (680 mg, 28.6 mmol, 4.0 eq), THE (25 mL), H₂O (5 mL). The reaction mixture was stirred for 1 hour at 25° C. The resulting mixture was then diluted with water (30 mL) and acidified to pH=3 with HCl (1 M). The precipitated solids were collected by filtration and dried under infrared light to give [(5-bromo-2-fluoro-3-methoxypyridin-4-yl)methoxy]acetic acid as a white solid (1.8 g, 85.7%). ¹H NMR (400 MHz, DMSO-d₆) δ 12.73 (s, 1H), 8.19 (d, J=1.5 Hz, 1H), 4.69 (s, 2H), 4.10 (s, 2H), 3.94 (d, J=2.0 Hz, 3H).

Synthesis of 2-[(5-bromo-2-fluoro-3-methoxypyridin-4-yl)methoxy]-N-methoxy-N-methylacetamide: Into a 250 mL 3-necked round-bottom flask, were placed [(5-bromo-2-fluoro-3-methoxypyridin-4-yl)methoxy]acetic acid (1.8 g, 6.1 mmol, 1.0 eq), DCM (20 mL). This was followed by the addition of CDI (1.4 g, 8.6 mmol, 1.4 eq) at 0° C. The resulting mixture was stirred for 30 minutes at 0° C. To the above mixture was added N,O-dimethylhydroxylamine (490 mg, 8.0 mmol, 1.3 eq), THE (40 mL) at 0° C. The resulting mixture was stirred for additional 6 hours at 25° C. The resulting mixture was then quenched by the addition of water (50 mL), and extracted with ethyl acetate (2×50 mL). The combined organic layers was washed with brine (2×50 mL), dried over anhydrous Na₂SO₄. After filtration, the filtrate was concentrated under vacuum. The crude residue was applied onto a silica gel column and eluted with ethyl acetate/petroleum ether=1:3 to give 2-[(5-bromo-2-fluoro-3-methoxypyridin-4-yl)methoxy]-N-methoxy-N-methylacetamide as a white solid (1.1 g, 53.3%). ¹H NMR (300 MHz, DMSO-d₆) δ 8.19 (d, J=1.8 Hz, 1H), 4.71 (s, 2H), 4.34 (s, 2H), 3.94 (d, J=2.1 Hz, 3H), 3.66 (s, 3H), 3.11 (s, 3H).

Synthesis of 7-fluoro-8-methoxy-1H,3H-pyrano[4,3-c]pyridin-4-one: Into a 100 mL 3-necked round-bottom flask under nitrogen atmosphere, were placed 2-[(5-bromo-2-fluoro-3-methoxypyridin-4-yl)methoxy]-N-methoxy-N-methylacetamide (1.1 g, 3.3 mmol, 1.0 eq), THE (11 mL). This was followed by the addition of n-BuLi (2.0 mL, 2.5 eq, 2.5 M) at −78° C. The resulting mixture was stirred for 4 hours at −78° C. The resulting mixture was then quenched by the addition of water (20 mL), and extracted with ethyl acetate (2×20 mL) and washed with brine (2×20 mL). The mixture was dried over anhydrous Na₂SO₄. After filtration, the filtrate was concentrated under vacuum. The crude residue was purified onto a silica gel column and eluted with ethyl acetate/petroleum ether=1:3 to give 7-fluoro-8-methoxy-1H,3H-pyrano[4,3-c]pyridin-4-one as a white solid (370 mg, 57.5%). ¹H NMR (300 MHz, DMSO-d₆) δ 8.46 (d, J=1.5 Hz, 1H), 4.99 (s, 2H), 4.38 (s, 2H), 3.96 (d, J=2.6 Hz, 3H).

Synthesis of 7-fluoro-8-methoxy-1H,3H,4H-pyrano[4,3-c]pyridin-4-ol: Into a 50 mL 3-necked round-bottom flask, were placed 7-fluoro-8-methoxy-1H,3H-pyrano[4,3-c]pyridin-4-one (370 mg, 1.9 mmol, 1.0 eq), MeOH (4 mL). This was followed by the addition of NaBH₄ (106 mg, 2.8 mmol, 1.5 eq) at 0° C. The resulting mixture was stirred for 2 hours at 25° C. The resulting mixture was then quenched by the addition of water (30 mL), and then extracted with dichloromethane (2×30 mL). The combined organic layers was washed with brine (2×30 mL). The mixture was dried over anhydrous Na₂SO₄. After filtration, the filtrate was concentrated under vacuum. The crude residue was purified onto a silica gel column and eluted with ethyl acetate/petroleum ether=1:3 to give 7-fluoro-8-methoxy-1H,3H,4H-pyrano[4,3-c]pyridin-4-ol as a white solid (360 mg, 96.3%). ¹H NMR (400 MHz, DMSO-d₆) δ 7.97 (d, J=1.7 Hz, 1H), 5.63 (d, J=6.0 Hz, 1H), 4.83-4.73 (m, 1H), 4.70-4.57 (m, 2H), 3.88 (d, J=2.4 Hz, 3H), 3.82 (dd, J=11.4, 4.0 Hz, 1H), 3.65 (dd, J=11.4, 5.5 Hz, 1H).

Synthesis of 4-chloro-7-fluoro-8-methoxy-1H,3H,4H-pyrano[4,3-c]pyridine: Into a 40 mL sealed tube, were placed 7-fluoro-8-methoxy-1H,3H,4H-pyrano[4,3-c]pyridin-4-ol (360 mg, 1.8 mmol, 1.0 eq), CHCl₃ (1 mL) at 0° C. To the above mixture was added SOCl₂ (5 mL) at 0° C. The resulting mixture was stirred for 4 hours at 25° C. The resulting mixture was then quenched by the addition of water (30 mL), and then extracted with dichloromethane (2×30 mL). The combined organic layers was washed with brine (2×30 mL), dried over anhydrous Na₂SO₄. After filtration, the filtrate was concentrated under vacuum. The crude residue was purified onto a silica gel column and eluted with ethyl acetate/petroleum ether=1:3 to give 4-chloro-7-fluoro-8-methoxy-1H,3H,4H-pyrano[4,3-c]pyridine as a white solid (320 mg, 81.4%). ¹H NMR (300 MHz, DMSO-d₆) δ 8.04 (d, J=1.5 Hz, 1H), 5.48 (d, J=2.7 Hz, 1H), 5.02-4.92 (m, 1H), 4.78-4.65 (m, 1H), 4.14-3.98 (m, 2H), 3.91 (d, J=2.7 Hz, 3H).

Synthesis of (3R)-1-[(4S)-7-fluoro-8-methoxy-1H,3H,4H-pyrano[4,3-c]pyridin-4-yl]-3-(2-isopropoxyphenyl)piperazine (assumed) & (3R)-1-[(4R)-7-fluoro-8-methoxy-1H,3H,4H-pyrano[4,3-c]pyridin-4-yl]-3-(2-isopropoxyphenyl)piperazine (assumed): Into an 8-mL sealed tube, were placed 4-chloro-7-fluoro-8-methoxy-1H,3H,4H-pyrano[4,3-c]pyridine (320 mg, 1.5 mmol, 1.0 eq), (2R)-2-(2-isopropoxyphenyl)piperazine (389 mg, 1.8 mmol, 1.2 eq), K₂CO₃ (610 mg, 4.4 mmol, 3.0 eq), CH₃CN (4 mL). The resulting solution was stirred for 2 days at 60° C. The resulting mixture was then quenched by the addition of water (30 mL), and then extracted with dichloromethane (2×30 mL). The combined organic layers was washed with brine (2×30 mL) and dried over anhydrous Na₂SO₄. After filtration, the filtrate was concentrated under vacuum. The crude residue was applied onto a silica gel column and eluted with dichloromethane/methanol=20:1 to give (3R)-1-[(4S)-7-fluoro-8-methoxy-1H,3H,4H-pyrano[4,3-c]pyridin-4-yl]-3-(2-isopropoxyphenyl)piperazine (assumed) as an off-white solid (60 mg, 10.2%) and (3R)-1-[(4R)-7-fluoro-8-methoxy-1H,3H,4H-pyrano[4,3-c]pyridin-4-yl]-3-(2-isopropoxyphenyl)piperazine (assumed) as an off-white solid (70 mg, 11.9%). LC-MS (ES, m/z) M+1: 402.

Synthesis of 4-[(4S)-4-[(3R)-3-(2-isopropoxyphenyl)piperazin-1-yl]-8-methoxy-1H,3H,4H-pyrano[4,3-c]pyridin-7-yl]morpholine (assumed): Into an 8-mL sealed tube, were placed (3R)-1-[(4S)-7-fluoro-8-methoxy-1H,3H,4H-pyrano[4,3-c]pyridin-4-yl]-3-(2-isopropoxyphenyl)piperazine (assumed) (60 mg, 0.1 mmol, 1.0 eq), morpholine (0.3 mL), DMSO (0.3 mL), K₂CO₃ (62 mg, 0.4 mmol, 3.0 eq). The resulting solution was stirred for 6 hours at 120° C. The resulting mixture was then quenched by the addition of water (20 mL), and extracted with dichloromethane (2×20 mL). The combined organic layers was washed with brine (2×20 mL) and dried over anhydrous Na₂SO₄. After filtration, the filtrate was concentrated under vacuum. The crude residue was applied onto a silica gel column and eluted with dichloromethane/methanol=8:1 to give 4-[(4S)-4-[(3R)-3-(2-isopropoxyphenyl)piperazin-1-yl]-8-methoxy-1H,3H,4H-pyrano[4,3-c]pyridin-7-yl]morpholine (assumed) as a white solid (20 mg, 28.6%). LC-MS (ES, m/z) M+1: 469.

Synthesis of 4-[(4R)-4-[(3R)-3-(2-isopropoxyphenyl)piperazin-1-yl]-8-methoxy-1H,3H,4H-pyrano[4,3-c]pyridin-7-yl]morpholine (assumed): Into an 8-mL sealed tube, were placed (3R)-1-[(4R)-7-fluoro-8-methoxy-1H,3H,4H-pyrano[4,3-c]pyridin-4-yl]-3-(2-isopropoxyphenyl)piperazine (assumed) (70 mg, 0.2 mmol, 1.0 eq), morpholine (0.3 mL), DMSO (0.3 mL), K₂CO₃ (72 mg, 0.5 mmol, 3.0 eq). The resulting solution was stirred for 6 hours at 120° C. The resulting mixture was then quenched by the addition of water (20 mL), and extracted with dichloromethane (2×20 mL). The combined organic layer was washed with brine (2×20 mL) and dried over anhydrous Na₂SO₄. After filtration, the filtrate was concentrated under vacuum. The crude residue was applied onto a silica gel column and eluted with dichloromethane/methanol=8:1 to give 4-[(4R)-4-[(3R)-3-(2-isopropoxyphenyl)piperazin-1-yl]-8-methoxy-1H,3H,4H-pyrano[4,3-c]pyridin-7-yl]morpholine (assumed) as a white solid (20 mg, 24.5%). LC-MS (ES, m/z) M+1: 469.

Synthesis of 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-4-{2-[(2R)-2-(2-isopropoxyphenyl)-4-[(4S)-8-methoxy-7-(morpholin-4-yl)-1H,3H,4H-pyrano[4,3-c]pyridin-4-yl]piperazin-1-yl]-7-azaspiro[3.5]nonan-7-yl}-N-[(3R)-5-nitro-3-(oxan-4-yl)-3,4-dihydro-2H-1,4-benzoxazin-7-ylsulfonyl]benzamide (assumed): Into an 8-mL sealed tube, were placed 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-N-[(3R)-5-nitro-3-(oxan-4-yl)-3,4-dihydro-2H-1,4-benzoxazin-7-ylsulfonyl]-4-{2-oxo-7-azaspiro[3.5]nonan-7-yl}benzamide (20 mg, 0.03 mmol, 1.0 eq), 4-[(4S)-4-[(3R)-3-(2-isopropoxyphenyl)piperazin-1-yl]-8-methoxy-1H,3H,4H-pyrano[4,3-c]pyridin-7-yl]morpholine (assumed) (14 mg, 0.03 mmol, 1.2 eq), ZnCl₂ (10 mg, 0.08 mmol, 3.0 eq), NaBH₃CN (5 mg, 0.08 mmol, 3.0 eq), MeOH (0.5 mL). The resulting solution was stirred for overnight at 70° C. in an oil bath. The crude product was purified by Flash-Prep-HPLC using the following conditions: Column: XBridge Shield RP18 OBD Column, 30*150 mm, 5 μm; Mobile Phase A: water (0.05% NH₃·H₂O), Mobile Phase B: CH₃CN; Flow rate: 60 mL/min; Gradient: 20% B to 50% B in 8 min, Wave Length: 254/220 nm. Finally, 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-4-{2-[(2R)-2-(2-isopropoxyphenyl)-4-[(4S)-8-methoxy-7-(morpholin-4-yl)-1H,3H,4H-pyrano[4,3-c]pyridin-4-yl]piperazin-1-yl]-7-azaspiro[3.5]nonan-7-yl}-N-[(3R)-5-nitro-3-(oxan-4-yl)-3,4-dihydro-2H-1,4-benzoxazin-7-ylsulfonyl]benzamide (assumed) was obtained as a yellow solid (2.7 mg, 8.7%). LC-MS (ES, m/z) M+1: 1267. ¹H NMR (400 MHz, Methanol-d₄) δ 8.11-8.01 (m, 2H), 7.74-7.52 (m, 1H), 7.48-7.39 (m, 1H), 7.38-7.26 (m, 1H), 7.25-7.16 (m, 1H), 6.98-6.84 (m, 4H), 6.65-6.64 (m, 2H), 6.01-5.94 (m, 1H), 4.91-4.86 (m, 1H), 4.69-4.59 (m, 1H), 4.58-4.52 (m, 1H), 4.41-4.33 (m, 1H), 4.26-3.94 (m, 7H), 3.88-3.75 (m, 8H), 3.68-3.61 (m, 2H), 3.50-3.28 (m, 9H), 3.11-2.92 (m, 6H), 2.85-2.62 (m, 4H), 2.23-2.12 (m, 1H), 1.97-1.39 (m, 12H), 1.38-1.32 (m, 9H), 1.23-1.14 (m, 1H).

### Example 251 Preparation of 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-4-{2-[(2R)-2-(2-isopropoxyphenyl)-4-[(4R)-8-methoxy-7-[(3S)-3-methylmorpholin-4-yl]-1H,3H,4H-pyrano[4,3-c]pyridin-4-yl]piperazin-1-yl]-7-azaspiro[3.5]nonan-7-yl}-N-[(3R)-5-nitro-3-(oxan-4-yl)-3,4-dihydro-2H-1,4-benzoxazin-7-ylsulfonyl]benzamide (assumed)

Synthesis of 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-4-{2-[(2R)-2-(2-isopropoxyphenyl)-4-[(4R)-8-methoxy-7-[(3S)-3-methylmorpholin-4-yl]-1H,3H,4H-pyrano[4,3-c]pyridin-4-yl]piperazin-1-yl]-7-azaspiro[3.5]nonan-7-yl}-N-[(3R)-5-nitro-3-(oxan-4-yl)-3,4-dihydro-2H-1,4-benzoxazin-7-ylsulfonyl]benzamide (assumed): Into an 8-mL sealed tube, were placed 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-N-[(3R)-5-nitro-3-(oxan-4-yl)-3,4-dihydro-2H-1,4-benzoxazin-7-ylsulfonyl]-4-{2-oxo-7-azaspiro[3.5]nonan-7-yl}benzamide (20 mg, 0.03 mmol, 1.0 eq), (3S)-4-[(4R)-4-[(3R)-3-(2-isopropoxyphenyl)piperazin-1-yl]-8-methoxy-1H,3H,4H-pyrano[4,3-c]pyridin-7-yl]-3-methylmorpholine (assumed) (14 mg, 0.03 mmol, 1.2 eq), ZnCl₂ (10 mg, 0.08 mmol, 3.0 eq), NaBH₃CN (5 mg, 0.08 mmol, 3.0 eq), MeOH (0.5 mL). The resulting solution was stirred for overnight at 70° C. in an oil bath. The crude product was purified by Flash-Prep-HPLC using the following conditions: Column: XBridge Shield RP18 OBD Column, 30*150 mm, 5 μm; Mobile Phase A: water (0.05% NH₃·H₂O), Mobile Phase B: CH₃CN; Flow rate: 60 mL/min; Gradient: 20% B to 50% B in 8 min, Wave Length: 254/220 nm. Finally, 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-4-{2-[(2R)-2-(2-isopropoxyphenyl)-4-[(4R)-8-methoxy-7-[(3S)-3-methylmorpholin-4-yl]-1H,3H,4H-pyrano[4,3-c]pyridin-4-yl]piperazin-1-yl]-7-azaspiro[3.5]nonan-7-yl}-N-[(3R)-5-nitro-3-(oxan-4-yl)-3,4-dihydro-2H-1,4-benzoxazin-7-ylsulfonyl]benzamide (assumed) was obtained as a yellow solid (2.0 mg, 6.4%). LC-MS (ES, m/z) M+1: 1267. ¹H NMR (400 MHz, Methanol-d₄) δ 8.20-8.00 (m, 2H), 7.70-7.54 (m, 1H), 7.44-7.25 (m, 2H), 7.22-7.12 (m, 1H), 6.97-6.82 (m, 4H), 6.65-6.64 (m, 2H), 6.01-5.95 (m, 1H), 4.82-4.78 (m, 1H), 4.56-4.45 (m, 2H), 4.39-4.30 (m, 1H), 4.25-3.93 (m, 6H), 3.90-3.77 (m, 6H), 3.74-3.59 (m, 6H), 3.52-3.39 (m, 4H), 3.16-2.94 (m, 6H), 2.81-2.69 (m, 1H), 2.61-2.33 (m, 3H), 1.98-1.34 (m, 18H), 1.33-1.16 (m, 5H), 1.11-0.84 (m, 4H).

### Example 252 Preparation of 4-(2-((R)-4-((4,4-dimethyl-8-((S)-3-methylmorpholino)-3,4-dihydro-2H-pyrano[2,3-c]pyridin-5-yl)methyl)-2-(o-tolyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide

Synthesis of (3S)-4-{4,4-dimethyl-2H,3H-pyrano[2,3-c]pyridin-8-yl}-3-methylmorpholine: Into a 200-mL round flask and maintained with an inert atmosphere of nitrogen, were placed 8-chloro-4,4-dimethyl-2H,3H-pyrano[2,3-c]pyridine (5.0 g, 25.3 mmol, 1.0 eq), (3S)-3-methylmorpholine (2.6 g, 25.3 mmol, 1.0 eq), t-BuOK (8.5 g, 75.9 mmol, 3.0 eq), Pd-PEPPSI-IPentC (250 mg), dioxane (50 mL). The resulting solution was stirred for 16 hours at 110° C. in an oil bath. The resulting mixture was then quenched by the addition of water (100 mL), and then extracted with ethyl acetate (2×100 mL). The combined organic layers was washed with brine (2×100 mL) and dried over anhydrous Na₂SO₄. After filtration, the filtrate was concentrated under vacuum. The crude residue was purified onto a silica gel column and eluted with ethyl acetate/petroleum ether=1:1 to give (3S)-4-{4,4-dimethyl-2H,3H-pyrano[2,3-c]pyridin-8-yl}-3-methylmorpholine as a light yellow solid (3.4 g, 51.2%). ¹H NMR (300 MHz, DMSO-d₆) δ 7.69 (d, J=5.1 Hz, 1H), 6.89 (d, J=5.1 Hz, 1H), 4.23-4.12 (m, 2H), 4.06-3.94 (m, 1H), 3.83-3.65 (m, 2H), 3.62-3.54 (m, 1H), 3.51-3.46 (m, 1H), 3.31-3.26 (m, 1H), 3.15-3.08 (m, 1H), 1.81-1.77 (m, 2H), 1.27 (s, 6H), 1.01 (d, J=6.6 Hz, 3H).

Synthesis of (S)-5-bromo-4,4-dimethyl-8-(3-methylmorpholino)-3,4-dihydro-2H-pyrano[2,3-c]pyridine: Into a 100 round-bottom flask, were placed (3S)-4-{4,4-dimethyl-2H,3H-pyrano[2,3-c]pyridin-8-yl}-3-methylmorpholine (3.2 g, 12.2 mmol, 1.0 eq), DMF (40 mL), NBS (4.3 g, 24.4 mmol, 2.0 eq). The resulting solution was stirred for 16 hours at 80° C. The resulting mixture was then quenched by the addition of water (120 mL), and then extracted with ethyl acetate (2×100 mL). The combined organic layer was washed with brine (2×100 mL) and dried over anhydrous Na₂SO₄. After filtration, the filtrate was concentrated under vacuum. The crude residue was purified onto a silica gel column and eluted with ethyl acetate/petroleum ether=1:2 to give (S)-5-bromo-4,4-dimethyl-8-(3-methylmorpholino)-3,4-dihydro-2H-pyrano[2,3-c]pyridine as a light pink oil (1.6 g, 38.4%). ¹H NMR (300 MHz, DMSO-d₆) δ 7.84 (s, 1H), 4.20-4.11 (m, 2H), 4.00-3.95 (m, 1H), 3.79-3.66 (m, 2H), 3.60-3.47 (m, 2H), 3.29-3.24 (m, 1H), 3.17-3.09 (m, 1H), 1.88-1.85 (m, 2H), 1.51 (s, 6H), 1.03 (d, J=6.7 Hz, 3H).

Synthesis of (S)-4,4-dimethyl-8-(3-methylmorpholino)-5-vinyl-3,4-dihydro-2H-pyrano[2,3-c]pyridine: Into a 40-mL sealed tube, were added (S)-5-bromo-4,4-dimethyl-8-(3-methylmorpholino)-3,4-dihydro-2H-pyrano[2,3-c]pyridine (1.5 g, 4.3 mmol, 1.0 eq), K₂CO₃ (1.8 g, 13.2 mmol, 3.0 eq), dioxane (15 mL), H₂O (15 mL), 4,4,5,5-tetramethyl-2-vinyl-1,3,2-dioxaborolane(1.1 g, 8.6 mmol, 2.0 eq) and tBuXPhos Pd G3 (0.2 g, 0.2 mmol, 0.04 eq) at 25° C. The resulting mixture was stirred for 16 hours at 80° C. under N₂ atmosphere. The reaction was quenched by the addition of H₂O (30 mL), and then extracted with ethyl acetate (3×30 mL). The combined organic layers were washed with brine (30 mL), dried over anhydrous Na₂SO₄. After filtration, the filtrate was concentrated under vacuum. The crude residue was purified onto a silica gel column and eluted with ethyl acetate/petroleum ether=1:2 to give (S)-4,4-dimethyl-8-(3-methylmorpholino)-5-vinyl-3,4-dihydro-2H-pyrano[2,3-c]pyridine as a yellow solid (1.0 g, 38.4%). ¹H NMR (300 MHz, Chloroform-d) δ 7.84 (d, J=0.8 Hz, 1H), 7.09 (dd, J=17.2, 10.8 Hz, 1H), 5.48 (d, J=17.1 Hz, 1H), 5.37-5.20 (m, 1H), 4.29-4.17 (m, 2H), 4.01-3.84 (m, 2H), 3.90-3.74 (m, 1H), 3.64 (dd, J=11.3, 3.9 Hz, 1H), 3.56 (s, 1H), 3.21 (s, 1H), 1.87 (dd, J=6.3, 4.0 Hz, 2H), 1.46 (d, J=1.7 Hz, 6H), 1.37-1.22 (m, 1H), 1.18 (s, 3H).

Synthesis of (S)-4,4-dimethyl-8-(3-methylmorpholino)-3,4-dihydro-2H-pyrano[2,3-c]pyridine-5-carbaldehyde: Into a 100 mL bottom flask were added (S)-4,4-dimethyl-8-(3-methylmorpholino)-5-vinyl-3,4-dihydro-2H-pyrano[2,3-c]pyridine (1.0 g, 3.3 mmol, 1.0 eq), NalO₄ (2.4 g, 9.9 mmol, 3.0 eq), K₂OsO₄.2H₂O (1.2 g, 2.4 mmol, 0.7 eq), t-BuOH (6 mL), THE (6 mL), and H₂O (1 mL) at 0° C. The resulting mixture was stirred for 16 hours at 25° C. under N₂ atmosphere. The reaction was quenched by the addition of Na₂SO₃ (aq.) (100 mL), and then extracted with ethyl acetate (3×50 mL). The combined organic layers were washed with brine (50 mL), dried over anhydrous Na₂SO₄. After filtration, the filtrate was concentrated under vacuum. The crude residue was applied onto a silica gel column and eluted with ethyl acetate/petroleum ether=1:3 to give (S)-4,4-dimethyl-8-(3-methylmorpholino)-3,4-dihydro-2H-pyrano[2,3-c]pyridine-5-carbaldehyde as a yellow solid (700 mg, 59.5%). ¹H NMR (400 MHz, Chloroform-d) δ 10.12 (s, 1H), 8.27 (s, 1H), 4.21 (dd, J=6.2, 4.2 Hz, 2H), 3.94 (dd, J=12.6, 9.8 Hz, 2H), 3.83 (dd, J=11.3, 3.1 Hz, 1H), 3.72 (ddd, J=13.5, 11.4, 2.3 Hz, 2H), 3.53 (ddd, J=13.4, 11.5, 3.6 Hz, 1H), 1.89 (dd, J=6.3, 4.2 Hz, 2H), 1.55 (d, J=3.2 Hz, 7H), 1.34 (d, J=6.8 Hz, 3H).

Synthesis of 4,4-dimethyl-8-((S)-3-methylmorpholino)-5-(((R)-3-(o-tolyl)piperazin-1-yl)methyl)-3,4-dihydro-2H-pyrano[2,3-c]pyridine: Into a 40-mL sealed-tube, were placed (R)-2-(o-tolyl)piperazine (150 mg, 0.5 mmol, 1.0 eq), (S)-4,4-dimethyl-8-(3-methylmorpholino)-3,4-dihydro-2H-pyrano[2,3-c]pyridine-5-carbaldehyde (118 mg, 0.5 mmol, 1.1 eq), MeOH(4 mL), NaBH₃CN (152 mg, 1.4 mmol, 3.0 eq), ZnCl₂(307 mg, 1.4 mmol, 3.0 eq). The resulting solution was stirred for 16 hours at 70° C. The resulting mixture was then quenched by the addition of water (40 mL), and then extracted with ethyl acetate (2×40 mL). The combined organic layer was washed with brine (2×40 mL) and the organic layers combined. The mixture was dried over anhydrous Na₂SO₄. After filtration, the filtrate was concentrated under vacuum. The crude residue was purified onto a silica gel column and eluted with ethyl acetate/petroleum ether=1:2 to give 4,4-dimethyl-8-((S)-3-methylmorpholino)-5-(((R)-3-(o-tolyl)piperazin-1-yl)methyl)-3,4-dihydro-2H-pyrano[2,3-c]pyridine as a light yellow solid (100 mg, 40.1%). LC-MS (ES, m/z) M+1: 451.

Synthesis of 4-(2-((R)-4-((4,4-dimethyl-8-((S)-3-methylmorpholino)-3,4-dihydro-2H-pyrano[2,3-c]pyridin-5-yl)methyl)-2-(o-tolyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide: Into an 8-mL sealed tube, were placed 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-N-[(3R)-5-nitro-3-(oxan-4-yl)-3,4-dihydro-2H-1,4-benzoxazin-7-ylsulfonyl]-4-{2-oxo-7-azaspiro[3.5]nonan-7-yl}benzamide (40 mg, 0.04 mmol, 1.0 eq), 4,4-dimethyl-8-((S)-3-methylmorpholino)-5-(((R)-3-(o-tolyl)piperazin-1-yl)methyl)-3,4-dihydro-2H-pyrano[2,3-c]pyridine (22 mg, 0.06 mmol, 1.2 eq), ZnCl₂ (20 mg, 0.16 mmol, 3.0 eq), NaBH₃CN (10 mg, 0.16 mmol, 3.0 eq), MeOH (1 mL). The resulting solution was stirred for 16 hours at 50° C. in an oil bath. The resulting mixture was then quenched by the addition of water (30 mL) and then extracted with dichloromethane (2×30 mL). The combined organic layer was washed with brine (2×30 mL) and dried over anhydrous Na₂SO₄. After filtration, the filtrate was concentrated under vacuum. The crude residue was applied onto a silica gel column and eluted with dichloromethane/methanol=15:1 to give 4-(2-((R)-4-((4,4-dimethyl-8-((S)-3-methylmorpholino)-3,4-dihydro-2H-pyrano[2,3-c]pyridin-5-yl)methyl)-2-(o-tolyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide as a yellow solid (16 mg, 27.6%). LC-MS (ES, m/z) M+1: 1248. ¹H NMR (400 MHz, Chloroform-d) δ 12.92 (s, 1H), 12.62 (s, 1H), 8.45 (s, 1H), 8.40-8.27 (m, 4H), 8.14-8.13 (m, 1H), 8.06-8.05 (m, 2H), 7.46 (d, J=2.2 Hz, 1H), 7.36-7.33 (m, 1H), 7.19 (s, 1H), 7.13-7.06 (m, 1H), 6.89-6.80 (m, 1H), 6.68 (s, 1H), 6.44-6.41 (m, 1H), 6.13-6.11 (m, 1H), 4.59-4.40 (m, 2H), 4.22 (s, 1H), 4.16-4.02 (m, 5H), 3.79-3.70 (m, 7H), 3.55-3.45 (m, 3H), 3.43-3.41 (m, 1H), 3.39-3.36 (m, 4H), 3.07-3.05 (m, 9H), 2.85-2.81 (m, 2H), 2.74-2.71 (m, 2H), 2.39-2.36 (m, 4H), 2.08-2.05 (m, 3H), 1.86-1.84 (m, 4H), 1.78-1.76 (m, 1H), 1.68-1.67 (m, 1H), 1.43-1.41 (m, 8H), 1.27-1.25 (m, 1H), 1.15-1.13 (m, 4H), 0.84-0.82 (m, 1H).

### Example 253 Preparation of 4-(2-((R)-2-(2-isopropoxyphenyl)-4-(pyridin-3-ylmethyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide

Synthesis of (R)-3-(2-isopropoxyphenyl)-1-(pyridin-3-ylmethyl)piperazine: Into an 8 mL vial were added 3-pyridinecarboxaldehyde (48 mg, 0.4 mmol, 1.0 eq), (2R)-2-(2-isopropoxyphenyl)piperazine (98 mg, 0.4 mmol, 1.0 eq), NaBH₃CN (140 mg, 2.2 mmol, 5.0 eq), ZnCl₂ (305 mg, 2.2 mmol, 5.0 eq) and MeOH (1 mL). The resulting mixture was stirred for 6 hours at 25° C. The reaction was quenched with water (5 mL) and then extracted with CH₂Cl₂ (3×10 mL). The combined organic layers were washed with brine (10 mL), dried over anhydrous Na₂SO₄. After filtration, the filtrate was concentrated under vacuum. The crude residue was purified by Prep-TLC (CH₂Cl₂/MeOH=10:1) to give (R)-3-(2-isopropoxyphenyl)-1-(pyridin-3-ylmethyl)piperazine as a light yellow oil (55 mg, 39.4%). LC-MS (ES, m/z) M+1: 312. ¹H NMR (300 MHz, Chloroform-d) δ 8.58 (d, J=2.2 Hz, 1H), 8.52 (dd, J=4.7, 1.6 Hz, 1H), 7.73 (d, J=7.8 Hz, 1H), 7.45-7.40 (m, 1H), 7.30-7.14 (m, 2H), 6.91 (t, J=7.5 Hz, 1H), 6.83 (d, J=8.2 Hz, 1H), 4.54-4.50 (m, 1H), 4.25-4.21 (m, 1H), 3.76-3.61 (m, 1H), 3.48-3.44 (m, 1H), 3.18-3.05 (m, 2H), 2.97-2.94 (m, 1H), 2.88-2.83 (m, 1H), 2.32-2.30 (m, 1H), 2.02-2.00 (m, 2H), 1.33-1.30 m, 3H), 1.20-1.15 (m, 3H).

Synthesis of 4-(2-((R)-2-(2-isopropoxyphenyl)-4-(pyridin-3-ylmethyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide: Into an 8 mL vial were added (R)-3-(2-isopropoxyphenyl)-1-(pyridin-3-ylmethyl)piperazine (7 mg, 0.1 mmol, 1.0 eq), 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-N-[(3R)-5-nitro-3-(oxan-4-yl)-3,4-dihydro-2H-1,4-benzoxazin-7-ylsulfonyl]-4-{2-oxo-7-azaspiro[3.5]nonan-7-yl}benzamide (18 mg, 0.1 mmol, 1.0 eq), NaBH₃CN (7 mg, 0.1 mmol, 5.0 eq), ZnCl₂ (15 mg, 0.1 mmol, 5.0 eq) and MeOH (1 mL). The resulting mixture was stirred for 4 hours at 70° C. The reaction was quenched with water (5 mL) and extracted with CH₂Cl₂ (3×10 mL). The combined organic layers were washed with brine (5 mL), dried over anhydrous Na₂SO₄. After filtration, the filtrate was concentrated under vacuum. The crude residue was purified by Prep-TLC (CH₂Cl₂/MeOH=19:1) to give 4-(2-((R)-2-(2-isopropoxyphenyl)-4-(pyridin-3-ylmethyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide as a yellow solid (13 mg, 52.1%). LC-MS (ES, m/z) M+1: 1110. ¹H NMR (300 MHz, Chloroform-d) δ 12.63 (s, 1H), 8.56 (s, 1H), 8.47-8.40 (m, 3H), 8.31 (d, J=2.2 Hz, 1H), 8.12-8.11 (m, 1H), 7.66-7.60 (m, 1H), 7.46 (d, J=2.2 Hz, 1H), 7.20-7.19 (m, 4H), 7.16 (s, 1H), 7.09 (t, J=3.0 Hz, 1H), 6.89-6.77 (m, 3H), 6.69 (s, 1H), 6.44 (d, J=2.4 Hz, 1H), 6.14-6.10 (m, 1H), 4.57-4.39 (m, 3H), 4.10-4.05 (m, 3H), 4.02-4.00 (m, 1H), 3.96-3.94 (m, 1H), 3.88-3.86 (m, 1H), 3.53-3.51 (m, 3H), 3.50-3.34 (m, 5H), 3.14-3.03 (m, 4H), 2.96-2.90 (m, 3H), 2.65-2.63 (m, 1H), 2.32-2.30 (m, 2H), 2.20-2.19 (m, 1H), 1.79-1.70 (m, 6H), 1.57-1.47 (m, 1H), 1.48-1.45 (m, 6H), 1.33-1.30 (m, 3H), 1.27-1.21 (m, 3H).

### Example 254 Preparation of 4-(2-((R)-2-(2-isopropoxyphenyl)-4-((6-methylpyridin-3-yl)methyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide

Synthesis of (R)-3-(2-isopropoxyphenyl)-1-((6-methylpyridin-3-yl)methyl)piperazine: Into a 40 mL vial were added 6-methylpyridine-3-carbaldehyde (80 mg, 0.6 mmol, 1.0 eq), (2R)-2-(2-isopropoxyphenyl)piperazine (145 mg, 0.6 mmol, 1.0 eq), NaBH₃CN (207 mg, 3.3 mmol, 5.0 eq), ZnCl₂ (450 mg, 3.3 mmol, 5.0 eq) and MeOH (2 mL). The resulting mixture was stirred for 5 hours at 25° C. The reaction was quenched with water (10 mL) and extracted with CH₂Cl₂ (3×10 mL). The combined organic layers were washed with brine (5 mL), dried over anhydrous Na₂SO₄. After filtration, the filtrate was concentrated under vacuum. The crude residue was purified by Prep-TLC (CH₂Cl₂/MeOH=10:1) to give (R)-3-(2-isopropoxyphenyl)-1-((6-methylpyridin-3-yl)methyl)piperazine as a light yellow oil (50 mg, 23.2%). LC-MS (ES, m/z) M+1: 326. ¹H NMR (300 MHz, Chloroform-d) δ 8.43 (d, J=2.3 Hz, 1H), 7.61-7.60 (m, 1H), 7.44-7.40 (m, 1H), 7.19-7.15 (m, 1H), 7.12-7.11 (m, 1H), 6.96-6.86 (m, 1H), 6.83 (d, J=8.3 Hz, 1H), 4.52-4.48 (m, 1H), 4.22-4.20 (m, 1H), 3.61-3.60 (m, 1H), 3.44-3.40 (m, 1H), 3.16-3.03 (m, 2H), 2.96-2.93 (m, 1H), 2.91-2.81 (m, 1H), 2.55-2.50 (m, 3H), 2.34-2.20 (m, 1H), 1.98-1.96 (m, 1H), 1.36-1.21 (m, 5H), 1.20-1.18 (m, 2H).

Synthesis of 4-(2-((R)-2-(2-isopropoxyphenyl)-4-((6-methylpyridin-3-yl)methyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide: Into an 8 mL vial were added (3R)-3-(2-isopropoxyphenyl)-1-[(6-methylpyridin-3-yl)methyl]piperazine (7 mg, 0.1 mmol, 1.0 eq), 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-N-[(3R)-5-nitro-3-(oxan-4-yl)-3,4-dihydro-2H-1,4-benzoxazin-7-ylsulfonyl]-4-{2-oxo-7-azaspiro[3.5]nonan-7-yl}benzamide (18 mg, 0.1 mmol, 1.0 eq), NaBH₃CN (6 mg, 0.1 mmol, 5.0 eq), ZnCl₂ (15 mg, 0.1 mmol, 5.0 eq) and MeOH (1 mL). The resulting mixture was stirred for 4 hours at 70° C. The reaction was quenched with water (10 mL) and then extracted with CH₂Cl₂ (3×10 mL). The combined organic layers were washed with brine (5 mL), dried over anhydrous Na₂SO₄. After filtration, the filtrate was concentrated under vacuum. The crude residue was purified by Prep-TLC (CH₂Cl₂/MeOH=18:1) to give 4-(2-((R)-2-(2-isopropoxyphenyl)-4-((6-methylpyridin-3-yl)methyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide as a yellow solid (15 mg, 60.3%). LC-MS (ES, m/z) M+1: 1125. ¹H NMR (300 MHz, Chloroform-d) δ 12.63 (s, 1H), 8.43-8.40 (m, 3H), 8.31 (d, J=2.2 Hz, 1H), 8.12-8.10 (m, 1H), 7.55-7.51 (m, 1H), 7.50-7.39 (m, 2H), 7.21-7.05 (m, 3H), 6.89-6.76 (m, 3H), 6.69 (s, 1H), 6.43 (d, J=2.4 Hz, 1H), 6.14-6.10 (m, 1H), 4.47-4.40 (m, 3H), 4.16-3.93 (m, 5H), 3.88-3.84 (m, 1H), 3.55-3.34 (m, 7H), 3.08-3.00 (m, 4H), 2.96-2.90 (m, 2H), 2.92-2.77 (m, 1H), 2.64-2.60 (m, 1H), 2.54-2.50 (m, 3H), 2.28-2.24 (m, 2H), 1.79-1.70 (m, 1H), 1.67-1.60 (m, 11H), 1.42-1.40 (m, 1H), 1.27-1.25 (m, 10H).

### Example 255 Preparation of 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-4-{2-[(2R)-2-(2-isopropoxyphenyl)-4-[(6-isopropylpyridin-3-yl)methyl]piperazin-1-yl]-7-azaspiro[3.5]nonan-7-yl}-N-[(3R)-5-nitro-3-(oxan-4-yl)-3,4-dihydro-2H-1,4-benzoxazin-7-ylsulfonyl]benzamide

Synthesis of 6-isopropylpyridine-3-carbaldehyde: Into a 100 mL 3-necked round-bottom flask under nitrogen atmosphere, were placed 5-bromo-2-isopropylpyridine (600 mg, 3.0 mmol, 1.0 eq), THE (6 mL). This was followed by the addition of n-BuLi (288 mg, 4.5 mmol, 1.5 eq) at −78° C. The resulting mixture was stirred for 30 minutes at −78° C. To the above mixture was added DMF (307 mg, 4.2 mmol, 1.4 eq) at −78° C. The resulting mixture was stirred for additional 30 minutes at −78° C. The resulting mixture was then quenched by the addition of water (20 mL), and then extracted with ethyl acetate (2×20 mL). The combined organic layer was washed with brine (2×20 mL). The mixture was dried over anhydrous Na₂SO₄. After filtration, the filtrate was concentrated under vacuum. The crude residue was purified onto a silica gel column and eluted with ethyl acetate/petroleum ether=1:1 to give 6-isopropylpyridine-3-carbaldehyde as a colorless oil (200 mg, 44.7%). ¹H NMR (400 MHz, DMSO-d₆) δ 10.07 (s, 1H), 9.02-8.97 (m, 1H), 8.16 (dd, J=8.0, 2.2 Hz, 1H), 7.50 (d, J=8.0 Hz, 1H), 3.12 (p, J=6.8 Hz, 1H), 1.26 (d, J=6.8 Hz, 6H).

Synthesis of (3R)-3-(2-isopropoxyphenyl)-1-[(6-isopropylpyridin-3-yl)methyl]piperazine: Into an 8-mL sealed tube, were placed 6-isopropylpyridine-3-carbaldehyde (200 mg, 1.3 mmol, 1.0 eq), (2R)-2-(2-isopropoxyphenyl)piperazine (354 mg, 1.6 mmol, 1.2 eq), ZnCl₂ (548 mg, 4.0 mmol, 3.0 eq), NaBH₃CN (253 mg, 4.0 mmol, 3.0 eq), MeOH (2 mL). The resulting solution was stirred for 2 hours at 25° C. The resulting mixture was then quenched by the addition of water (20 mL), and then extracted with dichloromethane (2×20 mL). The combined organic layers was washed with brine (2×20 mL) and dried over anhydrous Na₂SO₄. After filtration, the filtrate was concentrated under vacuum. The crude residue was applied onto a silica gel column and eluted with dichloromethane/methanol=20:1 to give (3R)-3-(2-isopropoxyphenyl)-1-[(6-isopropylpyridin-3-yl)methyl]piperazine as a light yellow oil (150 mg, 31.7%). LC-MS (ESI, m/z) M+1: 354.

Synthesis of 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-4-{2-[(2R)-2-(2-isopropoxyphenyl)-4-[(6-isopropylpyridin-3-yl)methyl]piperazin-1-yl]-7-azaspiro[3.5]nonan-7-yl}-N-[(3R)-5-nitro-3-(oxan-4-yl)-3,4-dihydro-2H-1,4-benzoxazin-7-ylsulfonyl]benzamide: Into an 8-mL sealed tube, were placed 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-N-[(3R)-5-nitro-3-(oxan-4-yl)-3,4-dihydro-2H-1,4-benzoxazin-7-ylsulfonyl]-4-{2-oxo-7-azaspiro[3.5]nonan-7-yl}benzamide (20 mg, 0.03 mmol, 1.0 eq), (3R)-3-(2-isopropoxyphenyl)-1-[(6-isopropylpyridin-3-yl)methyl]piperazine (10 mg, 0.03 mmol, 1.2 eq), ZnCl₂ (10 mg, 0.08 mmol, 3.0 eq), NaBH₃CN (5 mg, 0.08 mmol, 3.0 eq), MeOH (0.5 mL). The resulting solution was stirred for overnight at 70° C. in an oil bath. The crude product was purified by Flash-Prep-HPLC using the following conditions: Column: XBridge Shield RP18 OBD Column, 30*150 mm, 5 μm; Mobile Phase A: water (0.05% NH₃·H₂O), Mobile Phase B: CH₃CN; Flow rate: 60 mL/min; Gradient: 20% B to 50% B in 8 min, Wave Length: 254/220 nm. Finally, 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-4-{2-[(2R)-2-(2-isopropoxyphenyl)-4-[(6-isopropylpyridin-3-yl)methyl]piperazin-1-yl]-7-azaspiro[3.5]nonan-7-yl}-N-[(3R)-5-nitro-3-(oxan-4-yl)-3,4-dihydro-2H-1,4-benzoxazin-7-ylsulfonyl]benzamide was obtained as a yellow solid (6 mg, 21.2%). LC-MS (ESI, m/z) M+1: 1152. ¹H NMR (300 MHz, Methanol-d₄) δ 8.40 (s, 1H), 8.10-8.00 (m, 1H), 7.82-7.57 (m, 2H), 7.51-7.37 (m, 1H), 7.35-7.14 (m, 3H), 7.04-6.83 (m, 4H), 6.72-6.49 (m, 2H), 6.03-5.95 (m, 1H), 4.66-4.56 (m, 1H), 4.31-3.85 (m, 9H), 3.69-3.39 (m, 8H), 3.18-2.89 (m, 9H), 2.68-2.23 (m, 5H), 1.99-1.39 (m, 12H), 1.36-1.20 (m, 12H).

### Example 256 Preparation of 4-(2-((R)-4-((6-(tert-butyl)pyridin-3-yl)methyl)-2-(2-isopropoxyphenyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide

Synthesis of 6-(tert-butyl)nicotinaldehyde: A solution of 5-bromo-2-tert-butylpyridine (500 mg, 2.3 mmol, 1.0 eq) in THE (8 mL) was treated within-BuLi (1 mL, 11.8 mmol, 5.0 eq) for 30 minutes at −78° C. under nitrogen atmosphere followed by the addition of DMF (1024 mg, 14.1 mmol, 6.0 eq) dropwise at −78° C. The reaction was quenched by the addition of sat. NH₄Cl (aq.) (5 mL), and then extracted with EtOAc (3×10 mL). The combined organic layers were washed with brine (5 mL), dried over anhydrous Na₂SO₄. After filtration, the filtrate was concentrated under vacuum. The crude residue was purified by silica gel column chromatography, eluted with ethyl acetate/petroleum ether=1:5 to give 6-(tert-butyl)nicotinaldehyde as a light yellow oil (300 mg, 78.7%). ¹H NMR (300 MHz, Chloroform-d) δ 10.09 (s, 1H), 9.02 (dd, J=2.2, 1.0 Hz, 1H), 8.11 (dd, J=8.3, 2.2 Hz, 1H), 7.53 (d, J=8.3 Hz, 1H), 1.41 (s, 9H).

Synthesis of (R)-1-((6-(tert-butyl)pyridin-3-yl)methyl)-3-(2-isopropoxyphenyl)piperazine: Into an 8 mL vial were added 6-(tert-butyl)nicotinaldehyde (100 mg, 0.6 mmol, 1.0 eq), (2R)-2-(2-isopropoxyphenyl)piperazine (134 mg, 0.6 mmol, 1.0 eq), NaBH₃CN (192 mg, 3.1 mmol, 5.0 eq), ZnCl₂ (417 mg, 3.1 mmol, 5.0 eq) and MeOH (2 mL). The resulting mixture was stirred for 6 hours at 25° C. The reaction was quenched with water (10 mL) and then extracted with CH₂Cl₂ (3×10 mL). The combined organic layers were washed with brine (5 mL), dried over anhydrous Na₂SO₄. After filtration, the filtrate was concentrated under vacuum. The crude residue was purified by Prep-TLC (CH₂Cl₂/MeOH=10:1) to give (R)-1-((6-(tert-butyl)pyridin-3-yl)methyl)-3-(2-isopropoxyphenyl)piperazine as a light yellow oil (30 mg, 13.2%). LC-MS (ES, m/z) M+1: 369. ¹H NMR (300 MHz, Chloroform-d) δ 8.49 (d, J=2.3 Hz, 1H), 7.64-7.61 (m, 1H), 7.46 (d, J=7.5 Hz, 1H), 7.31-7.30 (m, 1H), 7.25-7.13 (m, 1H), 6.90-6.85 (m, 1H), 6.82-6.80 (m, 1H), 4.52-4.50 (m, 1H), 4.24-4.20 (m, 1H), 3.65-3.64 (m, 1H), 3.43-3.40 (m, 1H), 3.15-3.14 (m, 2H), 3.12-3.10 (m, 1H), 2.98-2.96 (m, 1H), 2.90-2.88 (m, 1H), 2.34-2.30 (m, 1H), 2.01-2.00 (m, 1H), 1.40-1.25 (m, 12H), 1.17-1.14 (m, 3H).

Synthesis of 4-(2-((R)-4-((6-(tert-butyl)pyridin-3-yl)methyl)-2-(2-isopropoxyphenyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide: Into an 8 mL vial were added (R)-1-((6-(tert-butyl)pyridin-3-yl)methyl)-3-(2-isopropoxyphenyl)piperazine (8 mg, 0.1 mmol, 1.0 eq), 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-N-[(3R)-5-nitro-3-(oxan-4-yl)-3,4-dihydro-2H-1,4-benzoxazin-7-ylsulfonyl]-4-{2-oxo-7-azaspiro[3.5]nonan-7-yl}benzamide (17 mg, 0.1 mmol, 1.0 eq), NaBH₃CN (6 mg, 0.1 mmol, 5.0 eq), ZnCl₂ (14 mg, 0.1 mmol, 5.0 eq) and MeOH (1 mL). The resulting mixture was stirred for 4 hours at 70° C. The reaction was quenched with water (10 mL) and extracted with CH₂Cl₂ (3×10 mL). The combined organic layers were washed with brine (5 mL), dried over anhydrous Na₂SO₄. After filtration, the filtrate was concentrated under vacuum. The crude residue was purified by Prep-TLC (CH₂Cl₂/MeOH=18:1) to give 4-(2-((R)-4-((6-(tert-butyl)pyridin-3-yl)methyl)-2-(2-isopropoxyphenyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide as a yellow solid (14 mg, 55.9%). LC-MS (ES, m/z) M+1: 1166. ¹H NMR (300 MHz, Chloroform-d) δ 12.64 (s, 1H), 8.46-8.40 (m, 3H), 8.39 (s, 1H), 8.31 (d, J=2.1 Hz, 1H), 8.12-8.11 (m, 1H), 7.58-7.55 (m, 1H), 7.50-7.38 (m, 2H), 7.28-7.24 (m, 1H), 7.15-7.14 (m, 1H), 7.13-7.05 (m, 1H), 6.92-6.76 (m, 3H), 6.70 (s, 1H), 6.44-6.40 (m, 1H), 6.14-6.11 (m, 1H), 4.50-4.45 (m, 3H), 4.16-4.02 (m, 5H), 3.98-3.94 (m, 1H), 3.88-3.83 (m, 1H), 3.61-3.39 (m, 8H), 3.37-3.36 (m, 1H), 3.14-3.03 (m, 4H), 3.03-2.84 (m, 4H), 2.65-2.61 (m, 1H), 2.25-2.20 (m, 3H), 1.81-1.72 (m, 5H), 1.49-1.46 (m, 4H), 1.42-1.40 (m, 1H), 1.39-1.26 (m, 13H), 1.25-1.19 (m, 3H).

### Example 257 Preparation of 4-(2-((R)-4-((6-cyclopropylpyridin-3-yl)methyl)-2-(2-isopropoxyphenyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide

Synthesis of 6-cyclopropylnicotinaldehyde: A solution of 5-bromo-2-cyclopropylpyridine (1.0 g, 5.1 mmol, 1.0 eq) in THE (10 mL) was treated with n-BuLi (2 mL (2.5 M), 24.4 mmol, 4.8 eq) for 30 minutes at −78° C. under nitrogen atmosphere, which was followed by the addition of DMF (2.2 g, 30.2 mmol, 6.0 eq) dropwise at −78° C. The reaction was quenched by the addition of sat. NH₄Cl (aq.) (8 mL) and then extracted with EtOAc (3×10 mL). The combined organic layers were washed with brine (5 mL), dried over anhydrous Na₂SO₄. After filtration, the filtrate was concentrated under vacuum. The crude residue was purified by silica gel column chromatography, eluted with ethyl acetate/petroleum ether=1:5 to give 6-cyclopropylnicotinaldehyde as a light yellow oil (300 mg, 40.7%). ¹H NMR (300 MHz, Chloroform-d) δ 10.04 (s, 1H), 8.88 (d, J=2.2 Hz, 1H), 8.03 (dd, J=8.1, 2.1 Hz, 1H), 7.31 (d, J=8.1 Hz, 1H), 2.22-2.03 (m, 1H), 1.26-1.08 (m, 3H), 1.01-0.82 (m, 1H).

Synthesis of (R)-1-((6-cyclopropylpyridin-3-yl)methyl)-3-(2-isopropoxyphenyl)piperazine: Into an 8 mL vial were added 6-cyclopropylnicotinaldehyde (90 mg, 0.6 mmol, 1.0 eq), (2R)-2-(2-isopropoxyphenyl)piperazine (134 mg, 0.6 mmol, 1.0 eq), NaBH₃CN (192 mg, 3.1 mmol, 5.0 eq), ZnCl₂ (416 mg, 3.0 mmol, 5.0 eq) and MeOH (2 mL). The resulting mixture was stirred for 6 hours at 25° C. The reaction was quenched with water (10 mL) and extracted with CH₂Cl₂ (3×10 mL). The combined organic layers were washed with brine (5 mL), dried over anhydrous Na₂SO₄. After filtration, the filtrate was concentrated under vacuum. The crude residue was purified by Prep-TLC (CH₂Cl₂/MeOH=10:1) to give (R)-1-((6-cyclopropylpyridin-3-yl)methyl)-3-(2-isopropoxyphenyl)piperazine as a light yellow oil (30 mg, 13.6%). LC-MS (ES, m/z) M+1: 352. ¹H NMR (300 MHz, Chloroform-d) δ 8.45-8.33 (m, 1H), 7.63-7.51 (m, 1H), 7.43 (d, J=7.6 Hz, 1H), 7.14-7.10 (m, 2H), 6.95-6.78 (m, 2H), 4.67-4.62 (m, 1H), 4.53-4.50 (m, 1H), 4.21-4.19 (m, 1H), 3.60-3.58 (m, 1H), 3.41-3.40 (m, 1H), 3.12 (s, 1H), 2.96-2.81 (m, 1H), 2.86-2.84 (m, 1H), 2.28-2.26 (m, 2H), 1.41-1.25 (m, 3H), 1.19-1.17 (m, 3H), 0.99-0.88 (m, 6H).

Synthesis of 4-(2-((R)-4-((6-cyclopropylpyridin-3-yl)methyl)-2-(2-isopropoxyphenyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide: Into an 8 mL vial were added (R)-1-((6-cyclopropylpyridin-3-yl)methyl)-3-(2-isopropoxyphenyl)piperazine (8 mg, 0.1 mmol, 1.0 eq), 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-N-[(3R)-5-nitro-3-(oxan-4-yl)-3,4-dihydro-2H-1,4-benzoxazin-7-ylsulfonyl]-4-{2-oxo-7-azaspiro[3.5]nonan-7-yl}benzamide (18 mg, 0.1 mmol, 1.0 eq), NaBH₃CN (7 mg, 0.5 mmol, 5.0 eq), ZnCl₂ (15 mg, 0.1 mmol, 5.0 eq) and MeOH (1 mL). The resulting mixture was stirred for 4 hours at 70° C. The reaction was quenched with water (10 mL) and then extracted with CH₂Cl₂ (3×10 mL). The combined organic layers were washed with brine (5 mL), dried over anhydrous Na₂SO₄. After filtration, the filtrate was concentrated under vacuum. The crude residue was purified by Prep-TLC (CH₂Cl₂/MeOH=19:1) to give 4-(2-((R)-4-((6-cyclopropylpyridin-3-yl)methyl)-2-(2-isopropoxyphenyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide as a yellow solid (13 mg, 49.9%). LC-MS (ES, m/z) M+1: 1150. ¹H NMR (300 MHz, Chloroform-d) δ 12.61 (s, 1H), 8.44-8.40 (m, 3H), 8.35-8.25 (m, 2H), 8.10-8.08 (m, 1H), 7.44-7.41 (m, 3H), 7.14-7.13 (m, 1H), 7.11-7.00 (m, 2H), 6.80-6.76 (m, 4H), 6.67 (s, 1H), 6.41 (d, J=2.4 Hz, 1H), 6.11-6.08 (m, 1H), 4.50-4.49 (m, 2H), 4.40-4.37 (m, 1H), 4.14-3.91 (m, 5H), 3.84-3.81 (m, 1H), 3.48-3.44 (m, 2H), 3.43-3.37 (m, 8H), 3.36-3.31 (m, 2H), 3.05-3.00 (m, 6H), 2.92-2.86 (m, 5H), 2.59-2.54 (m, 1H), 2.27-2.20 (m, 2H), 2.07-1.92 (m, 1H), 1.84-1.71 (m, 1H), 1.48-1.44 (m, 2H), 1.28-1.14 (m, 8H), 0.96-0.90 (m, 5H).

### Example 258 Preparation of 4-(2-((R)-2-(2-isopropoxyphenyl)-4-((6-methoxypyridin-3-yl)methyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide

Synthesis of (R)-3-(2-isopropoxyphenyl)-1-((6-methoxypyridin-3-yl)methyl)piperazine: Into a 40 mL vial were added 6-methoxypyridine-3-carbaldehyde (60 mg, 0.4 mmol, 1.0 eq), (2R)-2-(2-isopropoxyphenyl)piperazine (96 mg, 0.4 mmol, 1.0 eq), NaBH₃CN (137 mg, 2.1 mmol, 5.0 eq), ZnCl₂ (298 mg, 2.1 mmol, 5.0 eq) and MeOH (3 mL). The resulting mixture was stirred for 6 hours at 25° C. The reaction was quenched with water (10 mL) and then extracted with CH₂Cl₂ (3×10 mL). The combined organic layers were washed with brine (5 mL), dried over anhydrous Na₂SO₄. After filtration, the filtrate was concentrated under vacuum. The crude residue was purified by Prep-TLC (CH₂Cl₂/MeOH=15:1) to give (R)-3-(2-isopropoxyphenyl)-1-((6-methoxypyridin-3-yl)methyl)piperazine as a light yellow oil (33 mg, 22.0%). LC-MS (ES, m/z) M+1: 342. ¹H NMR (300 MHz, Chloroform-d) δ 8.06 (d, J=2.4 Hz, 1H), 7.64 (d, J=8.0 Hz, 1H), 7.46 (d, J=7.4 Hz, 1H), 7.20 (t, J=7.5 Hz, 1H), 6.96-6.81 (m, 2H), 6.75-6.70 (m, 1H), 4.55-4.50 (m, 1H), 4.26-4.21 (m, 1H), 3.95-3.90 (m, 3H), 3.59-3.56 (m, 2H), 3.42-3.40 (m, 1H), 3.14-3.10 (m, 1H), 2.98-2.93 (m, 1H), 2.88-2.86 (m, 1H), 2.39-2.27 (m, 2H), 2.06-2.00 (m, 1H), 1.28-1.19 (m, 6H).

Synthesis of 4-(2-((R)-2-(2-isopropoxyphenyl)-4-((6-methoxypyridin-3-yl)methyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide: Into an 8 mL vial were added (R)-3-(2-isopropoxyphenyl)-1-((6-methoxypyridin-3-yl)methyl)piperazine (8 mg, 0.1 mmol, 1.0 eq), 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-N-[(3R)-5-nitro-3-(oxan-4-yl)-3,4-dihydro-2H-1,4-benzoxazin-7-ylsulfonyl]-4-{2-oxo-7-azaspiro[3.5]nonan-7-yl}benzamide (19 mg, 0.1 mmol, 1.0 eq), NaBH₃CN (7 mg, 0.1 mmol, 5.0 eq), ZnCl₂ (15 mg, 0.1 mmol, 5.0 eq) and MeOH (1 mL). The resulting mixture was stirred for 4 hours at 70° C. The reaction was quenched with water (10 mL) and then extracted with CH₂Cl₂ (3×10 mL). The combined organic layers were washed with brine (5 mL), dried over anhydrous Na₂SO₄. After filtration, the filtrate was concentrated under vacuum. The crude residue was purified by Prep-TLC (CH₂Cl₂/MeOH=17:1) to give 4-(2-((R)-2-(2-isopropoxyphenyl)-4-((6-methoxypyridin-3-yl)methyl)piperazin-1-yl)-7-azaspiro[3.5]nonan-7-yl)-N-(((R)-5-nitro-3-(tetrahydro-2H-pyran-4-yl)-3,4-dihydro-2H-benzo[b][1,4]oxazin-7-yl)sulfonyl)-2-((5aS,9aR)-6,7,9,9a-tetrahydro-1H-pyrano[3,4-b]pyrrolo[3′,2′:5,6]pyrido[3,2-e][1,4]oxazin-5(5aH)-yl)benzamide as a yellow solid (10 mg, 37.4%). LC-MS (ES, m/z) M+1: 1140. ¹H NMR (300 MHz, Chloroform-d) δ 12.63 (s, 1H), 8.45 (s, 1H), 8.40-8.28 (m, 3H), 8.13-8.10 (m, 1H), 8.03 (s, 1H), 7.56 (s, 1H), 7.50-7.38 (m, 2H), 7.12-7.10 (m, 2H), 6.89-6.76 (m, 4H), 6.71-6.58 (m, 3H), 6.43-6.40 (m, 1H), 6.14 (s, 1H), 4.53-4.52 (m, 3H), 4.51-4.41 (m, 1H), 4.16-4.06 (m, 4H), 4.06-4.03 (m, 2H), 4.01-3.94 (m, 2H), 3.92-3.90 (m, 4H), 3.88-3.86 (m, 1H), 3.55-3.53 (m, 2H), 3.50-3.34 (m, 10H), 3.06-2.99 (m, 6H), 2.95-2.90 (m, 2H), 2.62-2.60 (m, 1H), 2.28-2.26 (m, 1H), 2.16-2.14 (m, 1H), 1.79-1.75 (m, 2H), 1.48-1.44 (m, 2H), 1.40-1.38 (m, 2H), 1.30-1.26 (m, 3H).

### Example 259 Preparation of 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-4-{2-[(2R)-2-(2-isopropoxyphenyl)-4-{[6-(methylamino)pyridin-3-yl]methyl}piperazin-1-yl]-7-azaspiro[3.5]nonan-7-yl}-N-[(3R)-5-nitro-3-(oxan-4-yl)-3,4-dihydro-2H-1,4-benzoxazin-7-ylsulfonyl]benzamide

Synthesis of 6-(methylamino)pyridine-3-carbaldehyde: Into a 40 mL sealed tube, were placed 6-fluoropyridine-3-carbaldehyde (1.2 g, 9.6 mmol, 1.0 eq), methylamine (300 mg, 9.6 mmol, 1.0 eq), K₂CO₃ (4.0 g, 28.8 mmol, 3.0 eq), DMSO (12 mL). The resulting solution was stirred for overnight at 100° C. The resulting mixture was then quenched by the addition of water (20 mL), and extracted with dichloromethane (2×20 mL). The combined organic layers was washed with brine (2×20 mL) and dried over anhydrous Na₂SO₄. After filtration, the filtrate was concentrated under vacuum. The crude residue was applied onto a silica gel column and eluted with ethyl acetate/petroleum ether=1:3 to give 6-(methylamino)pyridine-3-carbaldehyde as a yellow solid (450 mg, 34.5%). ¹H NMR (300 MHz, DMSO-d₆) δ 9.67 (s, 1H), 8.51 (d, J=2.4 Hz, 1H), 7.74 (d, J=9.0 Hz, 1H), 7.67 (s, 1H), 6.54 (d, J=9.0 Hz, 1H), 2.87 (d, J=4.8 Hz, 3H).

Synthesis of 5-{[(3R)-3-(2-isopropoxyphenyl)piperazin-1-yl]methyl}-N-methylpyridin-2-amine: Into an 8 mL sealed tube, were placed 6-(methylamino)pyridine-3-carbaldehyde (200 mg, 1.5 mmol, 1.0 eq), (2R)-2-(2-isopropoxyphenyl)piperazine (324 mg, 1.5 mmol, 1.0 eq), ZnCl₂ (601 mg, 4.4 mmol, 3.0 eq), NaBH₃CN (277 mg, 4.4 mmol, 3.0 eq), MeOH (2 mL). The resulting solution was stirred for 2 hours at 25° C. The resulting mixture was quenched by the addition of water (30 mL), and then extracted with dichloromethane (2×30 mL). The combined organic layers was washed with brine (2×30 mL) and dried over anhydrous Na₂SO₄. After filtration, the filtrate was concentrated under vacuum. The crude residue was applied onto a silica gel column and eluted with dichloromethane/methanol=20:1 to give 5-{[(3R)-3-(2-isopropoxyphenyl)piperazin-1-yl]methyl}-N-methylpyridin-2-amine as a light-yellow oil (150 mg, 30.0%). LC-MS (ESI, m/z) M+1: 341.

Synthesis of 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-4-{2-[(2R)-2-(2-isopropoxyphenyl)-4-{[6-(methylamino)pyridin-3-yl]methyl}piperazin-1-yl]-7-azaspiro[3.5]nonan-7-yl}-N-[(3R)-5-nitro-3-(oxan-4-yl)-3,4-dihydro-2H-1,4-benzoxazin-7-ylsulfonyl]benzamide: Into an 8-mL sealed tube, were placed 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-N-[(3R)-5-nitro-3-(oxan-4-yl)-3,4-dihydro-2H-1,4-benzoxazin-7-ylsulfonyl]-4-{2-oxo-7-azaspiro[3.5]nonan-7-yl}benzamide (20 mg, 0.03 mmol, 1.0 eq), 5-{[(3R)-3-(2-isopropoxyphenyl)piperazin-1-yl]methyl}-N-methylpyridin-2-amine (10 mg, 0.03 mmol, 1.2 eq), ZnCl₂ (10 mg, 0.08 mmol, 3.0 eq), NaBH₃CN (5 mg, 0.08 mmol, 3.0 eq), MeOH (0.5 mL). The resulting solution was stirred for overnight at 70° C. in an oil bath. The crude product was purified by Flash-Prep-HPLC using the following conditions: Column: XBridge Shield RP18 OBD Column, 30*150 mm, 5 μm; Mobile Phase A: water (0.05% NH₃·H₂O), Mobile Phase B: CH₃CN; Flow rate: 60 mL/min; Gradient: 20% B to 50% B in 8 min, Wave Length: 254/220 nm. Finally, 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-4-{2-[(2R)-2-(2-isopropoxyphenyl)-4-{[6-(methylamino)pyridin-3-yl]methyl}piperazin-1-yl]-7-azaspiro[3.5]nonan-7-yl}-N-[(3R)-5-nitro-3-(oxan-4-yl)-3,4-dihydro-2H-1,4-benzoxazin-7-ylsulfonyl]benzamide was obtained as a yellow solid (6 mg, 21.5%). LC-MS (ESI, m/z) M+1: 1139. ¹H NMR (400 MHz, Methanol-d₄) δ 8.05-8.00 (m, 1H), 7.88-7.78 (m, 2H), 7.49-7.40 (m, 2H), 7.27-7.19 (m, 2H), 7.05-6.99 (m, 1H), 6.97-6.86 (m, 3H), 6.71-6.63 (m, 1H), 6.55-6.48 (m, 2H), 6.02-5.94 (m, 1H), 4.67-4.60 (m, 1H), 4.31-4.19 (m, 3H), 4.16-4.07 (m, 2H), 4.05-3.87 (m, 3H), 3.70-3.37 (m, 8H), 3.17-2.96 (m, 7H), 2.75-2.66 (m, 1H), 2.49-2.33 (m, 2H), 1.98-1.79 (m, 3H), 1.78-1.61 (m, 4H), 1.54-1.24 (m, 18H).

### Example 260 Preparation of 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-4-{2-[(2R)-2-(2-isopropoxyphenyl)-4-({6-[(3S)-3-methylmorpholin-4-yl]pyridin-3-yl}methyl)piperazin-1-yl]-7-azaspiro[3.5]nonan-7-yl}-N-[(3R)-5-nitro-3-(oxan-4-yl)-3,4-dihydro-2H-1,4-benzoxazin-7-ylsulfonyl]benzamide

Synthesis of 6-[(3S)-3-methylmorpholin-4-yl]pyridine-3-carbaldehyde: Into a 40-mL sealed tube, were placed 6-fluoropyridine-3-carbaldehyde (1.0 g, 8.0 mmol, 1.0 eq), (3S)-3-methylmorpholine (810 mg, 8.0 mmol, 1.0 eq), DIEA (3.1 g, 23.9 mmol, 3.0 eq), DMSO (10 mL). The resulting solution was stirred for overnight at 100° C. The resulting mixture was quenched by the addition of water (15 mL), and then extracted with dichloromethane (2×15 mL). The combined organics were washed with brine (2×15 mL) and dried over anhydrous Na₂SO₄. After filtration, the filtrate was concentrated under vacuum. The crude residue was applied onto a silica gel column and eluted with ethyl acetate/petroleum ether=1:3 to give 6-[(3S)-3-methylmorpholin-4-yl]pyridine-3-carbaldehyde as a yellow oil (1.6 g, 97.1%). LC-MS (ESI, m/z) M+1: 207.

Synthesis of (3S)-4-(5-{[(3R)-3-(2-isopropoxyphenyl)piperazin-1-yl]methyl}pyridin-2-yl)-3-methylmorpholine: Into an 8-mL sealed tube, were placed 6-[(3S)-3-methylmorpholin-4-yl]pyridine-3-carbaldehyde (200 mg, 1.0 mmol, 1.0 eq), (2R)-2-(2-isopropoxyphenyl)piperazine (214 mg, 1.0 mmol, 1.0 eq), ZnCl₂ (396 mg, 2.9 mmol, 3.0 eq), NaBH₃CN (183 mg, 2.9 mmol, 3.0 eq), MeOH (2 mL). The resulting solution was stirred for 2 hours at 25° C. The resulting mixture was then quenched by the addition of water (20 mL), and then extracted with dichloromethane (2×20 mL). The combined organic layers was washed with brine (2×20 mL) and dried over anhydrous Na₂SO₄. After filtration, the filtrate was concentrated under vacuum. The crude residue was applied onto a silica gel column and eluted with dichloromethane/methanol=20:1 to give (3S)-4-(5-{[(3R)-3-(2-isopropoxyphenyl)piperazin-1-yl]methyl}pyridin-2-yl)-3-methylmorpholine as a light-yellow solid (150 mg, 37.7%). LC-MS (ESI, m/z) M+1: 411.

Synthesis of 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-4-{2-[(2R)-2-(2-isopropoxyphenyl)-4-({6-[(3S)-3-methylmorpholin-4-yl]pyridin-3-yl}methyl)piperazin-1-yl]-7-azaspiro[3.5]nonan-7-yl}-N-[(3R)-5-nitro-3-(oxan-4-yl)-3,4-dihydro-2H-1,4-benzoxazin-7-ylsulfonyl]benzamide: Into an 8-mL sealed tube, were placed 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-N-[(3R)-5-nitro-3-(oxan-4-yl)-3,4-dihydro-2H-1,4-benzoxazin-7-ylsulfonyl]-4-{2-oxo-7-azaspiro[3.5]nonan-7-yl}benzamide (20 mg, 0.03 mmol, 1.0 eq), (3S)-4-(5-{[(3R)-3-(2-isopropoxyphenyl)piperazin-1-yl]methyl}pyridin-2-yl)-3-methylmorpholine (12 mg, 0.03 mmol, 1.2 eq), ZnCl₂ (10 mg, 0.08 mmol, 3.0 eq), NaBH₃CN (5 mg, 0.08 mmol, 3.0 eq), MeOH (0.5 mL). The resulting solution was stirred for overnight at 70° C. in an oil bath. The crude product was purified by Flash-Prep-HPLC using the following conditions: Column: XBridge Shield RP18 OBD Column, 30*150 mm, 5 μm; Mobile Phase A: water (0.05% NH₃·H₂O), Mobile Phase B: CH₃CN; Flow rate: 60 mL/min; Gradient: 20% B to 50% B in 8 min, Wave Length: 254/220 nm. Finally, 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-4-{2-[(2R)-2-(2-isopropoxyphenyl)-4-({6-[(3S)-3-methylmorpholin-4-yl]pyridin-3-yl}methyl)piperazin-1-yl]-7-azaspiro[3.5]nonan-7-yl}-N-[(3R)-5-nitro-3-(oxan-4-yl)-3,4-dihydro-2H-1,4-benzoxazin-7-ylsulfonyl]benzamide was obtained as a yellow solid (6 mg, 20.2%). LC-MS (ESI, m/z) M+1: 1209. ¹H NMR (300 MHz, Methanol-d₄) δ 8.07-7.98 (m, 2H), 7.84-7.74 (m, 1H), 7.62-7.50 (m, 1H), 7.47-7.39 (m, 1H), 7.34-7.17 (m, 2H), 7.04-6.84 (m, 4H), 6.79-6.62 (m, 2H), 6.55-6.48 (m, 1H), 6.02-5.94 (m, 1H), 4.68-4.58 (m, 1H), 4.33-3.94 (m, 10H), 3.82-3.71 (m, 4H), 3.69-3.39 (m, 8H), 3.19-3.01 (m, 9H), 2.73-2.32 (m, 5H), 1.77-1.60 (m, 4H), 1.56-1.39 (m, 6H), 1.35-1.15 (m, 12H).

### Example 261 Preparation of 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-4-{2-[(2R)-2-(2-isopropoxyphenyl)-4-{[6-(trifluoromethyl)pyridin-3-yl]methyl}piperazin-1-yl]-7-azaspiro[3.5]nonan-7-yl}-N-[(3R)-5-nitro-3-(oxan-4-yl)-3,4-dihydro-2H-1,4-benzoxazin-7-ylsulfonyl]benzamide

Synthesis of (3R)-3-(2-isopropoxyphenyl)-1-{[6-(trifluoromethyl)pyridin-3-yl]methyl}piperazine: Into an 8 mL sealed tube, were placed 6-(trifluoromethyl)pyridine-3-carbaldehyde (120 mg, 0.7 mmol, 1.0 eq), (2R)-2-(2-isopropoxyphenyl)piperazine (181 mg, 0.8 mmol, 1.2 eq), ZnCl₂ (280 mg, 2.1 mmol, 3.0 eq), NaBH₃CN (129 mg, 2.1 mmol, 3.0 eq), MeOH (2 mL). The resulting solution was stirred for 2 hours at 25° C. The resulting mixture was then quenched by the addition of water (30 mL), and then extracted with dichloromethane (2×30 mL). The combined organic layers was washed with brine (2×30 mL) and dried over anhydrous Na₂SO₄. After filtration, the filtrate was concentrated under vacuum. The crude residue was applied onto a silica gel column and eluted with dichloromethane/methanol=20:1 to give (3R)-3-(2-isopropoxyphenyl)-1-{[6-(trifluoromethyl)pyridin-3-yl]methyl}piperazine as a colorless oil (70 mg, 26.9%). LC-MS (ESI, m/z) M+1: 380.

Synthesis of 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-4-{2-[(2R)-2-(2-isopropoxyphenyl)-4-{[6-(trifluoromethyl)pyridin-3-yl]methyl}piperazin-1-yl]-7-azaspiro[3.5]nonan-7-yl}-N-[(3R)-5-nitro-3-(oxan-4-yl)-3,4-dihydro-2H-1,4-benzoxazin-7-ylsulfonyl]benzamide: Into an 8-mL sealed tube, were placed 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-N-[(3R)-5-nitro-3-(oxan-4-yl)-3,4-dihydro-2H-1,4-benzoxazin-7-ylsulfonyl]-4-{2-oxo-7-azaspiro[3.5]nonan-7-yl}benzamide (20 mg, 0.03 mmol, 1.0 eq), (3R)-3-(2-isopropoxyphenyl)-1-{[6-(trifluoromethyl)pyridin-3-yl]methyl}piperazine (11 mg, 0.03 mmol, 1.2 eq), ZnCl₂ (10 mg, 0.08 mmol, 3.0 eq), NaBH₃CN (5 mg, 0.08 mmol, 3.0 eq), MeOH (0.5 mL). The resulting solution was stirred for overnight at 70° C. in an oil bath. The crude product was purified by Flash-Prep-HPLC using the following conditions: Column: XBridge Shield RP18 OBD Column, 30*150 mm, 5 μm; Mobile Phase A: water (0.05% NH₃·H₂O), Mobile Phase B: CH₃CN; Flow rate: 60 mL/min; Gradient: 20% B to 50% B in 8 min, Wave Length: 254/220 nm. Finally, 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-4-{2-[(2R)-2-(2-isopropoxyphenyl)-4-{[6-(trifluoromethyl)pyridin-3-yl]methyl}piperazin-1-yl]-7-azaspiro[3.5]nonan-7-yl}-N-[(3R)-5-nitro-3-(oxan-4-yl)-3,4-dihydro-2H-1,4-benzoxazin-7-ylsulfonyl]benzamide was obtained as a yellow solid (6 mg, 20.7%). LC-MS (ESI, m/z) M+1: 1177. ¹H NMR (400 MHz, Methanol-d₄) δ 8.74-8.67 (m, 1H), 8.20-8.00 (m, 2H), 7.83-7.58 (m, 2H), 7.50-7.40 (m, 1H), 7.33-7.14 (m, 2H), 7.05-6.85 (m, 4H), 6.71-6.49 (m, 2H), 6.03-5.94 (m, 1H), 4.66-4.54 (m, 1H), 4.32-4.10 (m, 4H), 4.07-3.95 (m, 3H), 3.93-3.86 (m, 1H), 3.75-3.63 (m, 3H), 3.52-3.38 (m, 4H), 3.20-2.90 (m, 7H), 2.66-2.58 (m, 1H), 2.49-2.31 (m, 3H), 1.98-1.80 (m, 2H), 1.79-1.63 (m, 3H), 1.55-1.39 (m, 8H), 1.38-1.29 (m, 5H), 1.27-1.20 (m, 4H).

### Example 262 Preparation of 4-{2-[(2R)-4-[(6-cyanopyridin-3-yl)methyl]-2-(2-isopropoxyphenyl)piperazin-1-yl]-7-azaspiro[3.5]nonan-7-yl}-2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-N-[(3R)-5-nitro-3-(oxan-4-yl)-3,4-dihydro-2H-1,4-benzoxazin-7-ylsulfonyl]benzamide

Synthesis of 5-{[(3R)-3-(2-isopropoxyphenyl)piperazin-1-yl]methyl}pyridine-2-carbonitrile: Into an 8 mL sealed tube, were placed 5-formylpyridine-2-carbonitrile (150 mg, 1.1 mmol, 1.0 eq), (2R)-2-(2-isopropoxyphenyl)piperazine (300 mg, 1.4 mmol, 1.2 eq), ZnCl₂ (464 mg, 3.4 mmol, 3.0 eq), NaBH₃CN (214 mg, 3.4 mmol, 3.0 eq), MeOH (2 mL). The resulting solution was stirred for 2 hours at 25° C. The resulting mixture was quenched by the addition of water (30 mL), and then extracted with dichloromethane (2×30 mL). The combined organic layers was washed with brine (2×30 mL) and dried over anhydrous Na₂SO₄. After filtration, the filtrate was concentrated under vacuum. The crude residue was applied onto a silica gel column and eluted with dichloromethane/methanol=20:1 to give 5-{[(3R)-3-(2-isopropoxyphenyl)piperazin-1-yl]methyl}pyridine-2-carbonitrile as a white solid (100 mg, 26.2%). LC-MS (ESI, m/z) M+1: 337.

Synthesis of 4-{2-[(2R)-4-[(6-cyanopyridin-3-yl)methyl]-2-(2-isopropoxyphenyl)piperazin-1-yl]-7-azaspiro[3.5]nonan-7-yl}-2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-N-[(3R)-5-nitro-3-(oxan-4-yl)-3,4-dihydro-2H-1,4-benzoxazin-7-ylsulfonyl]benzamide: Into an 8-mL sealed tube, were placed 2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-N-[(3R)-5-nitro-3-(oxan-4-yl)-3,4-dihydro-2H-1,4-benzoxazin-7-ylsulfonyl]-4-{2-oxo-7-azaspiro[3.5]nonan-7-yl}benzamide (30 mg, 0.04 mmol, 1.0 eq), 5-{[(3R)-3-(2-isopropoxyphenyl)piperazin-1-yl]methyl}pyridine-2-carbonitrile (15 mg, 0.04 mmol, 1.2 eq), ZnCl₂ (15 mg, 0.1 mmol, 3.0 eq), NaBH₃CN (7 mg, 0.1 mmol, 3.0 eq), THE (0.5 mL). The resulting solution was stirred for overnight at 70° C. in an oil bath. The crude product was purified by Flash-Prep-HPLC using the following conditions: Column: XBridge Shield RP18 OBD Column, 30*150 mm, 5 μm; Mobile Phase A: water (0.05% NH₃·H₂O), Mobile Phase B: CH₃CN; Flow rate: 60 mL/min; Gradient: 20% B to 50% B in 8 min, Wave Length: 254/220 nm. Finally, 4-{2-[(2R)-4-[(6-cyanopyridin-3-yl)methyl]-2-(2-isopropoxyphenyl)piperazin-1-yl]-7-azaspiro[3.5]nonan-7-yl}-2-[(3R,8S)-2,5-dioxa-9,15,17-triazatetracyclo[8.7.0.0\hat{ }{3,8}.0\hat{ }{12,16}]heptadeca-1(10),11,13,16-tetraen-9-yl]-N-[(3R)-5-nitro-3-(oxan-4-yl)-3,4-dihydro-2H-1,4-benzoxazin-7-ylsulfonyl]benzamide was obtained as a yellow solid (6 mg, 14.4%). LC-MS (ESI, m/z) M+1: 1134. ¹H NMR (400 MHz, Methanol-d₄) δ 8.74-8.67 (m, 1H), 8.20-8.00 (m, 2H), 7.83-7.58 (m, 2H), 7.50-7.40 (m, 1H), 7.33-7.14 (m, 2H), 7.05-6.85 (m, 4H), 6.71-6.49 (m, 2H), 6.03-5.94 (m, 1H), 4.66-4.54 (m, 1H), 4.36-3.80 (m, 9H), 3.74-3.55 (m, 2H), 3.54-3.39 (m, 4H), 3.21-2.84 (m, 8H), 2.70-2.27 (m, 4H), 2.03-1.81 (m, 2H), 1.79-1.38 (m, 10H), 1.35-1.20 (m, 9H).

### Biological Example 1: Bcl-2 Competition Binding Assay

The fluorescence-labeled 23 amino acid peptide BH3 was purchased from CalBiochem (NLWAAQRYGRELRRMSDKFVD). An unbound Fluorescein labeled BH3 peptide emits random light with respect to the plane of polarization plane of excited light, resulting in a lower polarization degree (mP) value. When the peptide is bound to Bcl-2, the complex tumble slower and the emitted light can have a higher level of polarization, resulting in a higher mP value. This binding assay was performed in 96-well plate and with each assay contained 15 and 30 nM of labeled peptide and purified Bcl-2 protein (purchased from R&D Systems, Inc). The assay buffer contained 20 mM Hepes (pH 7.0), 50 mM KCl, 5 mM MgCl₂, 20 mM Na₂MoO₄, 0.1 mg/ml Bovine Gamma Globulin and 0.01% NP40. Compounds were diluted in DMSO and added to the final assay with concentration range from 20 μM to 2 nM. The polarization degree (mP) value was determined by BioTek Synergy II with background subtraction after 3 hours of incubation at room temperature. IC₅₀ was calculated using Prism software with sigmoidal dose-response curve fitting. ABT-737 was used as reference compound. Such assays, carried out with a range of doses of test compounds, allowed the determination of an approximate IC₅₀ value. Although the inhibitory properties of the compounds of the present invention vary with structural change as expected, the activity generally exhibited by these agents was in the range of IC₅₀=0.1-1000 nM.

### Biological Example 2: In Vitro Anti-Proliferation Assay in BCL-2-Dependent Acute Lymphoblastic Leukemia (ALL) Cell Line RS4; 11 with G101V Mutation

Cell antiproliferation was assayed by PerkinElmer ATPlite™ Luminescence Assay System. Briefly, the various test cancer cell lines were plated at a density of about 1×10⁴ cells per well in Costar 96-well plates, and were incubated with different concentrations of compounds for about 72 hours in medium supplemented with 10% FBS. One lyophilized substrate solution vial was then reconstituted by adding 5 mL of substrate buffer solution, and was agitated gently until the solution was homogeneous. About 50 μL of mammalian cell lysis solution was added to 100 μL of cell suspension per well of a microplate, and the plate was shaken for about five minutes in an orbital shaker at ˜700 rpm. This procedure was used to lyse the cells and to stabilize the ATP. Next, 50 μL substrate solution was added to the wells and microplate was shaken for five minutes in an orbital shaker at ˜700 rpm. Finally, the luminescence was measured by a PerkinElmer TopCount® Microplate Scintillation Counter. Such assays, carried out with a range of doses of test compounds, allowed the determination of the cellular anti-antiproliferative IC₅₀ of the compounds of the present invention.

The following table lists the IC₅₀ values of another study for certain compounds of the invention.

| Example | RS411-G101V |
|---|---|
| ABT-199(reference) | 783 |
| 99 | 38.5 |
| 100 | 16.1 |
| 101 | 12.9 |
| 102 | 18.0 |
| 103 | 35.1 |
| 104 | 6.9 |
| 105 | 19.3 |
| 106 | 32.9 |
| 108 | 7.2 |
| 109 | 17.2 |
| 110 | 23.8 |
| 111 | 10.7 |
| 112 | 10.9 |
| 113 | 8.2 |
| 114 | 11.8 |
| 115 | 27.3 |
| 116 | 28.7 |
| 117 | 13.2 |
| 118 | 4.4 |
| 119 | 14.6 |
| 120 | 147.0 |
| 121 | 47.9 |
| 122 | 10.0 |
| 123 | 2.9 |
| 124 | 2.0 |
| 125 | 13.9 |
| 126 | 11.7 |
| 127 | 8.1 |
| 128 | 22.2 |
| 129 | 33.9 |
| 130 | 13.1 |
| 131 | 34.6 |
| 132 | 32.2 |
| 133 | 32.4 |
| 134 | 18.8 |
| 135 | 11.2 |
| 136 | 35.6 |
| 137 | 41.5 |
| 138 | 1.7 |
| 139 | 3.5 |
| 140 | 6.4 |
| 141 | 35.9 |
| 142 | 8.0 |
| 143 | 7.0 |
| 144 | 7.3 |
| 145 | 6.1 |
| 146 | 8.2 |
| 147 | 31.9 |
| 148 | 6.3 |
| 149 | 28.3 |
| 150 | 14.1 |
| 151 | 19.8 |
| 152 | 28.3 |
| 153 | 47.1 |
| 154 | 27.6 |
| 155 | 91.8 |
| 156 | 12.2 |
| 157 | 31.8 |
| 158 | 21.2 |
| 159 | 7.5 |
| 160 | 22.8 |
| 161 | 18.1 |
| 162 | 49.9 |
| 163 | 27.9 |
| 164 | 48.5 |
| 165 | 10.9 |
| 166 | 585.3 |
| 167 | 75.8 |
| 168 | 64.3 |
| 169 | 3.82 |
| 170 | 205.1 |
| 171 | 220.0 |
| 172 | 1.4 |
| 173 | 1.0 |
| 174 | 46.5 |
| 175 | 67.2 |
| 176 | 22.2 |
| 177 | 4.0 |
| 178 | 40.9 |
| 179 | 31.2 |
| 180 | 7.5 |
| 181 | 9.6 |
| 182 | 58.1 |
| 183 | 73.3 |
| 184 | 40.6 |
| 185 | 111.7 |
| 186 | 10.6 |
| 187 | 2.04 |
| 188 | 11.11 |
| 189 | 36.77 |
| 190 | 11.11 |
| 191 | 14.16 |
| 192 | 2.41 |
| 193 | 73.26 |
| 194 | 2.53 |
| 195 | 94.48 |
| 196 | 92.31 |
| 197 | 328.5 |
| 198 | 144.90 |
| 199 | 50.36 |
| 200 | 82.60 |
| 201 | 231.80 |
| 202 | 15.14 |
| 203 | 68.58 |
| 204 | 27.51 |
| 205 | 21.29 |
| 206 | 229.30 |
| 207 | 26.93 |
| 208 | 72.67 |
| 209 | 16.34 |
| 210 | 37.82 |
| 211 | 87.22 |
| 212 | 33.47 |
| 213 | 23.76 |
| 214 | 384.60 |
| 215 | 48.04 |
| 216 | 30.60 |
| 217 | 94.94 |
| 218 | 121.00 |
| 219 | 56.30 |
| 220 | 261.30 |
| 221 | 130.00 |
| 222 | 240.00 |
| 223 | 223.40 |
| 224 | 136.20 |
| 225 | 179.80 |
| 226 | 62.61 |
| 227 | 119.10 |
| 228 | 202.20 |
| 230 | 104.30 |
| 231 | 94.66 |
| 232 | 84.25 |
| 233 | 4.64 |
| 234 | 180.70 |
| 235 | 2273 |
| 236 | 5.52 |
| 239 | 7.10 |
| 240 | 6.94 |
| 241 | 4.45 |
| 242 | 97.83 |
| 243 | 22.18 |
| 245 | 289.50 |
| 246 | 209.10 |
| 247 | 45.68 |
| 248 | 9.68 |
| 249 | 12.00 |
| 250 | 10.05 |
| 251 | 26.87 |
| 252 | 1.88 |
| 253 | 10.19 |
| 254 | 3.19 |
| 255 | 6.39 |
| 256 | 11.81 |
| 257 | 3.90 |
| 258 | 11.65 |
| 259 | 9.72 |
| 260 | 8.65 |
| 261 | 3.87 |
| 262 | 6.43 |

### Biological Example 3: Mice PK Study

The pharmacokinetics of compounds were evaluated in CD-1 mouse via Intravenous and Oral Administration. The IV dose was administered as a slow bolus in the Jugular vein, and oral doses were administered by gavage. The formulation for IV dosing was 5% DMSO in 20% HPBCD in water, and the PO formulation was 2.5% DMSO, 10% EtOH, 20% Cremphor EL, 67.5% DSW. The PK time point for the IV arm was 5, 15, 30 min, 1, 2, 4, 6, 8, 12, 24 hours post dose, and for PO arm was 15, 30 min, 1, 2, 4, 6, 8, 12, 24 hours post dose. Approximately 0.03 mL blood was collected at each time point. Blood of each sample was transferred into plastic micro centrifuge tubes containing EDTA-K2 and collect plasma within 15 min by centrifugation at 4000 g for 5 minutes in a 4° C. centrifuge. Plasma samples were stored in polypropylene tubes. The samples were stored in a freezer at −75±15° C. prior to analysis. Concentrations of compounds in the plasma samples were analyzed using a LC-MS/MS method. WinNonlin (Phoenix™, version 6.1) or other similar software was used for pharmacokinetic calculations. The following pharmacokinetic parameters were calculated, whenever possible from the plasma concentration versus time data: IV administration: C₀, CL, V_{d}, T_{1/2}, AUC_{inf}, AUCₗₐₛₜ, MRT, Number of Points for Regression; PO administration: Cₘₐₓ, Tₘₐₓ, T_{1/2}, AUC_{inf}, AUCₗₐₛₜ, F %, Number of Points for Regression. The pharmacokinetic data was described using descriptive statistics such as mean, standard deviation. Additional pharmacokinetic or statistical analysis was performed at the discretion of the contributing scientist, and was documented in the data summary.

### Biological Example 4: In Vivo Xenograft Studies

Compound of Example 3 is selected for in vivo studies in the BCL-2-dependent acute lymphoblastic leukemia (ALL) RS4; 11 xenograft model. The CB.17 SCID mice are obtained at age 6-8 weeks from vendors and acclimated for a minimum 7-day period. The cancer cells are then implanted into the nude mice. Depending on the specific tumor type, tumors are typically detectable about two weeks following implantation. When tumor sizes reach ˜100-200 mm³, the animals with appreciable tumor size and shape are randomly assigned into groups of 8 mice each, including one vehicle control group and treatment groups. Dosing varies depending on the purpose and length of each study, which typically proceeds for about 3-4 weeks. Tumor sizes and body weight are typically measured three times per week. In addition to the determination of tumor size changes, the last tumor measurement is used to generate the tumor size change ratio (T/C value), a standard metric developed by the National Cancer Institute for xenograft tumor evaluation. In most cases, % T/C values are calculated using the following formula: % T/C=100×ΔT/ΔC if ΔT>0. When tumor regression occurred (ΔT<0), however, the following formula is used: % T/T0=100×ΔT/T0. Values of <42% are considered significant.

What is claimed is:

1. A compound of Formula (1), or an N-oxide thereof, or a pharmaceutically acceptable salt, solvate, polymorph, tautomer, stereoisomer, an isotopic form, or a prodrug of said compound of Formula (1) or N-oxide thereof: 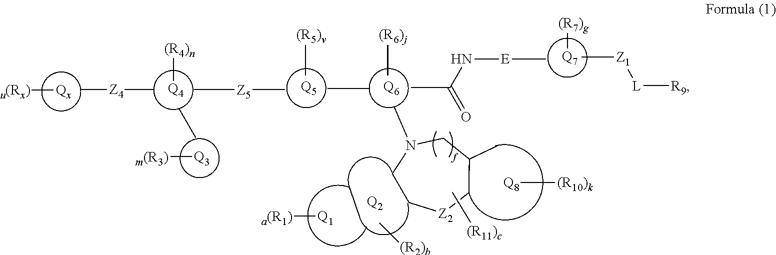
wherein
G is C(O), S(O₂), P(O)(Rₐ), or S(O)(NRₐ);
E is S(O₂), P(O)(Rₐ), S(O)(NRₐ) or C(O);
Q₁ is a cycloalkyl, cycloalkenyl, spirocycloalkyl, fused-carbocyclic, bridged-carbocyclic, heterocycloalkyl, heterocycloalkenyl, spiro-heterocyclic, fused-heterocyclic, bridged-heterocyclic, aryl, or heteroaryl, each of the aforementioned is optionally substituted with one or more independently selected R_{d} substituents;
Q₂ is a cycloalkyl, cycloalkenyl, spirocycloalkyl, fused-carbocyclic, bridged-carbocyclic, heterocycloalkyl, heterocycloalkenyl, spiro-heterocyclic, fused-heterocyclic, bridged-heterocyclic, aryl, or heteroaryl, each of the aforementioned is optionally substituted with one or more independently selected R_{d} substituents;
Q₃ is a cycloalkyl, cycloalkenyl, spirocycloalkyl, fused-carbocyclic, bridged-carbocyclic, heterocycloalkyl, heterocycloalkenyl, spiro-heterocyclic, fused-heterocyclic, bridged-heterocyclic, aryl, or heteroaryl, each of the aforementioned is optionally substituted with one or more independently selected R_{d} substituents;
Q₄ is a cycloalkyl, cycloalkenyl, spirocycloalkyl, fused-carbocyclic, bridged-carbocyclic, heterocycloalkyl, heterocycloalkenyl, spiro-heterocyclic, fused-heterocyclic, bridged-heterocyclic, aryl, or heteroaryl, each of the aforementioned is optionally substituted with one or more independently selected R_{d} substituents;
Q₅ is a cycloalkyl, cycloalkenyl, spirocycloalkyl, fused-carbocyclic, bridged-carbocyclic, heterocycloalkyl, heterocycloalkenyl, spiro-heterocyclic, fused-heterocyclic, bridged-heterocyclic, aryl, or heteroaryl, each of the aforementioned is optionally substituted with one or more independently selected R_{d} substituents;
Q₆ is a cycloalkyl, cycloalkenyl, spirocycloalkyl, fused-carbocyclic, bridged-carbocyclic, heterocycloalkyl, heterocycloalkenyl, spiro-heterocyclic, fused-heterocyclic, bridged-heterocyclic, aryl, or heteroaryl, each of the aforementioned is optionally substituted with one or more independently selected R_{d} substituents;
Q₇ is a cycloalkyl, cycloalkenyl, spirocycloalkyl, fused-carbocyclic, bridged-carbocyclic, heterocycloalkyl, heterocycloalkenyl, spiro-heterocyclic, fused-heterocyclic, bridged-heterocyclic, aryl, or heteroaryl, each of the aforementioned is optionally substituted with one or more independently selected R_{d} substituents;
Q₈ is a cycloalkyl, cycloalkenyl, spirocycloalkyl, fused-carbocyclic, bridged-carbocyclic, heterocycloalkyl, heterocycloalkenyl, spiro-heterocyclic, fused-heterocyclic, bridged-heterocyclic, aryl, or heteroaryl, each of the aforementioned is optionally substituted with one or more independently selected R_{d} substituents;
one or more (e.g., both) border ring atom(s) between ring Q₈ and the ring with Z₂ can be carbon or heteroatom(s);
Qₓ is a cycloalkyl, cycloalkenyl, spirocycloalkyl, fused-carbocyclic, bridged-carbocyclic, heterocycloalkyl, heterocycloalkenyl, spiro-heterocyclic, fused-heterocyclic, bridged-heterocyclic, aryl, or heteroaryl, each of the aforementioned is optionally substituted with one or more independently selected R_{d} substituents;
each R₁ is independently H, D, halo, cyano, nitro, alkyl, alkylene-Rₐ, alkylene-P(O)R_{b}R_{c}, alkenyl, alkynyl, —C(O)Rₐ, —C(O)NR_{b}R_{c}, —C(O)ORₐ, —NH(CH₂)ₚRₐ, —NR_{b}R_{c}, —NR_{b}C(O)R_{c}, =NR_{b}, —NR_{b}S(O)₂R_{c}, —N═S(O)R_{b}R_{c}, —ORₐ, —OC(O)Rₐ, ═O, —P(O)R_{b}R_{c}, —SRₐ, —S(O)Rₐ, —S(O)(NR_{b})R_{c}, —S(O)₂Rₐ, —S(O)₂NR_{b}R_{c}, cycloalkyl, cycloalkenyl, spirocycloalkyl, fused-carbocyclic, bridged-carbocyclic, heterocycloalkyl, heterocycloalkenyl, spiro-heterocyclic, fused-heterocyclic, bridged-heterocyclic, aryl, or heteroaryl, each of the aforementioned is optionally substituted with one or more independently selected R_{d} substituents;
each R₂ is independently H, D, halo, cyano, nitro, -alkyl, -alkylene-Rₐ, alkylene-P(O)R_{b}R_{c}, alkenyl, alkynyl, —C(O)Rₐ, —C(O)NR_{b}R_{c}, —C(O)ORₐ, —NH(CH₂)ₚRₐ, —NR_{b}R_{c}, —NR_{b}C(O)R_{c}, =NR_{b}, —NR_{b}S(O)₂R_{c}, —N═S(O)R_{b}R_{c}, —ORₐ, —OC(O)Rₐ, ═O, —P(O)R_{b}R_{c}, —SRₐ, —S(O)Rₐ, —S(O)(NR_{b})R_{c}, —S(O)₂Rₐ, —S(O)₂NR_{b}R_{c}, cycloalkyl, cycloalkenyl, spirocycloalkyl, fused-carbocyclic, bridged-carbocyclic, heterocycloalkyl, heterocycloalkenyl, spiro-heterocyclic, fused-heterocyclic, bridged-heterocyclic, aryl, or heteroaryl, each of the aforementioned is optionally substituted with one or more independently selected R_{d} substituents;
each R₃ is independently H, D, halo, cyano, nitro, alkyl, alkylene-Rₐ, alkylene-P(O)R_{b}R_{c}, alkenyl, alkynyl, —C(O)Rₐ, —C(O)NR_{b}R_{c}, —C(O)ORₐ, —NH(CH₂)ₚRₐ, —NR_{b}R_{c}, —NR_{b}C(O)R_{c}, =NR_{b}, —NR_{b}S(O)₂R_{c}, —N═S(O)R_{b}R_{c}, —ORₐ, —OC(O)Rₐ, ═O, —P(O)R_{b}R_{c}, —SRₐ, —S(O)Rₐ, —S(O)(NR_{b})R_{c}, —S(O)₂Rₐ, —S(O)₂NR_{b}R_{c}, cycloalkyl, cycloalkenyl, spirocycloalkyl, fused-carbocyclic, bridged-carbocyclic, heterocycloalkyl, heterocycloalkenyl, spiro-heterocyclic, fused-heterocyclic, bridged-heterocyclic, aryl, or heteroaryl, each of the aforementioned is optionally substituted with one or more independently selected R_{d} substituents; or
two R₃, taken together with the atom(s) to which they are attached, independently form a cycloalkyl or heterocycloalkyl, wherein each cycloalkyl or heterocycloalkyl is optionally and independently substituted with one or more independently selected R_{d} substituents;
each R₄ is independently H, D, halo, cyano, nitro, alkyl, alkylene-Rₐ, alkylene-P(O)R_{b}R_{c}, alkenyl, alkynyl, —C(O)Rₐ, —C(O)NR_{b}R_{c}, —C(O)ORₐ, —NH(CH₂)ₚRₐ, —NR_{b}R_{c}, —NR_{b}C(O)R_{c}, =NR_{b}, —NR_{b}S(O)₂R_{c}, —N═S(O)R_{b}R_{c}, —ORₐ, —OC(O)Rₐ, ═O, —P(O)R_{b}R_{c}, —SRₐ, —S(O)Rₐ, —S(O)(NR_{b})R_{c}, —S(O)₂Rₐ, —S(O)₂NR_{b}R_{c}, cycloalkyl, cycloalkenyl, spirocycloalkyl, fused-carbocyclic, bridged-carbocyclic, heterocycloalkyl, heterocycloalkenyl, spiro-heterocyclic, fused-heterocyclic, bridged-heterocyclic, aryl, or heteroaryl, each of the aforementioned is optionally substituted with one or more independently selected R_{d} substituents; or
two R₄, taken together with the atom(s) to which they are attached, independently form a cycloalkyl or heterocycloalkyl, wherein each cycloalkyl or heterocycloalkyl is optionally and independently substituted with one or more independently selected R_{d} substituents;
R₃ and R₄, taken together with the atom(s) to which they are attached, independently form a cycloalkyl or heterocycloalkyl, wherein each cycloalkyl or heterocycloalkyl is optionally and independently substituted with one or more independently selected R_{d} substituents;
each R₅ is independently H, D, halo, cyano, nitro, alkyl, alkylene-Rₐ, alkylene-P(O)R_{b}R_{c}, alkenyl, alkynyl, —C(O)Rₐ, —C(O)NR_{b}R_{c}, —C(O)ORₐ, —NH(CH₂)ₚRₐ, —NR_{b}R_{c}, —NR_{b}C(O)R_{c}, =NR_{b}, —NR_{b}S(O)₂R_{c}, —N═S(O)R_{b}R_{c}, —ORₐ, —OC(O)Rₐ, ═O, —P(O)R_{b}R_{c}, —SRₐ, —S(O)Rₐ, —S(O)(NR_{b})R_{c}, —S(O)₂Rₐ, —S(O)₂NR_{b}R_{c}, cycloalkyl, cycloalkenyl, spirocycloalkyl, fused-carbocyclic, bridged-carbocyclic, heterocycloalkyl, heterocycloalkenyl, spiro-heterocyclic, fused-heterocyclic, bridged-heterocyclic, aryl, or heteroaryl, each of the aforementioned is optionally substituted with one or more independently selected R_{d} substituents; or
R₄ and R₅, taken together with the atom(s) to which they are attached, independently form a cycloalkyl or heterocycloalkyl, wherein each cycloalkyl or heterocycloalkyl is optionally and independently substituted with one or more independently selected R_{d} substituents;
each R₆ is independently H, D, halo, cyano, nitro, alkyl, alkylene-Rₐ, alkylene-P(O)R_{b}R_{c}, alkenyl, alkynyl, —C(O)Rₐ, —C(O)NR_{b}R_{c}, —C(O)ORₐ, —NH(CH₂)ₚRₐ, —NR_{b}R_{c}, —NR_{b}C(O)R_{c}, =NR_{b}, —NR_{b}S(O)₂R_{c}, —N═S(O)R_{b}R_{c}, —ORₐ, —OC(O)Rₐ, ═O, —P(O)R_{b}R_{c}, —SRₐ, —S(O)Rₐ, —S(O)(NR_{b})R_{c}, —S(O)₂Rₐ, —S(O)₂NR_{b}R_{c}, cycloalkyl, cycloalkenyl, spirocycloalkyl, fused-carbocyclic, bridged-carbocyclic, heterocycloalkyl, heterocycloalkenyl, spiro-heterocyclic, fused-heterocyclic, bridged-heterocyclic, aryl, or heteroaryl, each of the aforementioned is optionally substituted with one or more independently selected R_{d} substituents; or
R₅ and R₆, taken together with the atom(s) to which they are attached, independently form a cycloalkyl or heterocycloalkyl, wherein each cycloalkyl or heterocycloalkyl is optionally and independently substituted with one or more independently selected R_{d} substituents;
each R₇ is independently H, D, halo, cyano, nitro, alkyl, alkylene-Rₐ, alkylene-P(O)R_{b}R_{c}, alkenyl, alkynyl, —C(O)Rₐ, —C(O)NR_{b}R_{c}, —C(O)ORₐ, —NH(CH₂)ₚRₐ, —NR_{b}R_{c}, —NR_{b}C(O)R_{c}, =NR_{b}, —NR_{b}S(O)₂R_{c}, —N═S(O)R_{b}R_{c}, —ORₐ, —OC(O)Rₐ, ═O, —P(O)R_{b}R_{c}, —SRₐ, —S(O)Rₐ, —S(O)(NR_{b})R_{c}, —S(O)₂Rₐ, —S(O)₂NR_{b}R_{c}, cycloalkyl, cycloalkenyl, spirocycloalkyl, fused-carbocyclic, bridged-carbocyclic, heterocycloalkyl, heterocycloalkenyl, spiro-heterocyclic, fused-heterocyclic, bridged-heterocyclic, aryl, or heteroaryl, each of the aforementioned is optionally substituted with one or more independently selected R_{d} substituents; or
R₇ and —Z₁-L-R₉, taken together with the atom(s) to which they are attached, independently form a cycloalkyl or heterocycloalkyl, wherein each cycloalkyl or heterocycloalkyl is optionally and independently substituted with one or more independently selected R_{d} substituents;
R₉ is H, D, halo, cyano, nitro, alkyl, alkylene-Rₐ, alkylene-P(O)R_{b}R_{c}, alkenyl, alkynyl, —C(O)Rₐ, —C(O)NR_{b}R_{c}, —C(O)ORₐ, —NH(CH₂)ₚRₐ, —NR_{b}R_{c}, —NR_{b}C(O)R_{c}, =NR_{b}, —NR_{b}S(O)₂R_{c}, —N═S(O)R_{b}R_{c}, —ORₐ, —OC(O)Rₐ, ═O, —P(O)R_{b}R_{c}, —SRₐ, —S(O)Rₐ, —S(O)(NR_{b})R_{c}, —S(O)₂Rₐ, —S(O)₂NR_{b}R_{c}, cycloalkyl, cycloalkenyl, spirocycloalkyl, fused-carbocyclic, bridged-carbocyclic, heterocycloalkyl, heterocycloalkenyl, spiro-heterocyclic, fused-heterocyclic, bridged-heterocyclic, aryl, or heteroaryl, each of the aforementioned is optionally substituted with one or more independently selected R_{d} substituents;
each R₁₀ is independently H, D, halo, cyano, nitro, alkyl, alkylene-Rₐ, alkylene-P(O)R_{b}R_{c}, alkenyl, alkynyl, —C(O)Rₐ, —C(O)NR_{b}R_{c}, —C(O)ORₐ, —NH(CH₂)ₚRₐ, —NR_{b}R_{c}, —NR_{b}C(O)R_{c}, =NR_{b}, —NR_{b}S(O)₂R_{c}, —N═S(O)R_{b}R_{c}, —ORₐ, —OC(O)Rₐ, ═O, —P(O)R_{b}R_{c}, —SRₐ, —S(O)Rₐ, —S(O)(NR_{b})R_{c}, —S(O)₂Rₐ, —S(O)₂NR_{b}R_{c}, cycloalkyl, cycloalkenyl, spirocycloalkyl, fused-carbocyclic, bridged-carbocyclic, heterocycloalkyl, heterocycloalkenyl, spiro-heterocyclic, fused-heterocyclic, bridged-heterocyclic, aryl, or heteroaryl, each of the aforementioned is optionally substituted with one or more independently selected R_{d} substituents; or
two R₁₀, taken together with the atom(s) to which they are attached, independently form a cycloalkyl or heterocycloalkyl, wherein each cycloalkyl or heterocycloalkyl is optionally and independently substituted with one or more independently selected R_{d} substituents;
R₁₁ is H, D, halo, cyano, nitro, alkyl, alkylene-Rₐ, alkylene-P(O)R_{b}R_{c}, alkenyl, alkynyl, —C(O)Rₐ, —C(O)NR_{b}R_{c}, —C(O)ORₐ, —NH(CH₂)ₚRₐ, —NR_{b}R_{c}, —NR_{b}C(O)R_{c}, =NR_{b}, —NR_{b}S(O)₂Rₐ, —N═S(O)R_{b}R_{c}, —ORₐ, —OC(O)Rₐ, ═O, —P(O)R_{b}R_{c}, —SRₐ, —S(O)Rₐ, —S(O)(NR_{b})R_{c}, —S(O)₂Rₐ, —S(O)₂NR_{b}R_{c}, cycloalkyl, cycloalkenyl, spirocycloalkyl, fused-carbocyclic, bridged-carbocyclic, heterocycloalkyl, heterocycloalkenyl, spiro-heterocyclic, fused-heterocyclic, bridged-heterocyclic, aryl, or heteroaryl, each of the aforementioned is optionally substituted with one or more independently selected R_{d} substituents; or
R₁₀ and R₁₁, taken together with the atom(s) to which they are attached, independently form a cycloalkyl or heterocycloalkyl, wherein each cycloalkyl or heterocycloalkyl is optionally and independently substituted with one or more independently selected R_{d} substituents;
each Rₓ is independently H, D, halo, cyano, nitro, alkyl, alkylene-Rₐ, alkylene-P(O)R_{b}R_{c}, alkenyl, alkynyl, —C(O)Rₐ, —C(O)NR_{b}R_{c}, —C(O)ORₐ, —NH(CH₂)ₚRₐ, —NR_{b}R_{c}, —NR_{b}C(O)R_{c}, =NR_{b}, —NR_{b}S(O)₂R_{c}, —N═S(O)R_{b}R_{c}, —ORₐ, —OC(O)Rₐ, ═O, —P(O)R_{b}R_{c}, —SRₐ, —S(O)Rₐ, —S(O)(NR_{b})R_{c}, —S(O)₂Rₐ, —S(O)₂NR_{b}R_{c}, cycloalkyl, cycloalkenyl, spirocycloalkyl, fused-carbocyclic, bridged-carbocyclic, heterocycloalkyl, heterocycloalkenyl, spiro-heterocyclic, fused-heterocyclic, bridged-heterocyclic, aryl, or heteroaryl, each of the aforementioned is optionally substituted with one or more independently selected R_{d} substituents; or
two Rₓ, taken together with the atom(s) to which they are attached, independently form a cycloalkyl or heterocycloalkyl, wherein each cycloalkyl or heterocycloalkyl is optionally and independently substituted with one or more independently selected R_{d} substituents;
R₄ and Rₓ, taken together with the atom(s) to which they are attached, independently form a cycloalkyl or heterocycloalkyl, wherein each cycloalkyl or heterocycloalkyl is optionally and independently substituted with one or more independently selected R_{d} substituents;
Z₁ is a bond, (CRₐR_{b})ₚ, a bivalent alkenyl group, or a bivalent alkynyl group, N(Rₐ), O, S, C(O), S(O₂), —O(CRₐR_{b})ₚ—, —N(Rₐ)(CRₐR_{b})ₚ—, OC(O), C(O)O, OSO₂, S(O₂)O, C(O)S, SC(O), C(O)C(O), C(O)N(Rₐ), N(Rₐ)C(O), S(O₂)N(Rₐ), N(Rₐ)S(O₂), OC(O)O, OC(O)S, OC(O)N(Rₐ), N(Rₐ)C(O)O, N(Rₐ)C(O)S, N(Rₐ)C(O)N(Rₐ), (CRₐR_{b})ₚN(Rₐ)(CRₐR_{b})_{q}, (CRₐR_{b})ₚN(Rₐ)C(O)(CRₐR_{b})_{q}, OC(O)N(R_{b})(CRₐR_{b})ₚ₊₁N(R_{b})(CRₐR_{b})_{q}, or (CRₐR_{b})ₚC(O)N(Rₐ)(CRₐR_{b})_{q};
Z₂ is a bond, (CRₐR_{b})ₚ, a bivalent alkenyl group, or a bivalent alkynyl group, N(Rₐ), O, S, C(O), S(O₂), —O(CRₐR_{b})ₚ—, —N(Rₐ)(CRₐR_{b})ₚ—, OC(O), C(O)O, OSO₂, S(O₂)O, C(O)S, SC(O), C(O)C(O), C(O)N(Rₐ), N(Rₐ)C(O), S(O₂)N(Rₐ), N(Rₐ)S(O₂), OC(O)O, OC(O)S, OC(O)N(Rₐ), N(Rₐ)C(O)O, N(Rₐ)C(O)S, N(Rₐ)C(O)N(Rₐ), (CRₐR_{b})ₚN(Rₐ)(CRₐR_{b})_{q}, (CRₐR_{b})ₚN(Rₐ)C(O)(CRₐR_{b})_{q}, OC(O)N(R_{b})(CRₐR_{b})ₚ₊₁N(R_{b})(CRₐR_{b})_{q}, or (CRₐR_{b})ₚC(O)N(Rₐ)(CRₐR_{b})_{q};
Z₄ is a bond, (CRₐR_{b})ₚ, a bivalent alkenyl group, or a bivalent alkynyl group, N(Rₐ), O, S, C(O), S(O₂), —O(CRₐR_{b})ₚ—, —N(Rₐ)(CRₐR_{b})ₚ—, OC(O), C(O)O, OSO₂, S(O₂)O, C(O)S, SC(O), C(O)C(O), C(O)N(Rₐ), N(Rₐ)C(O), S(O₂)N(Rₐ), N(Rₐ)S(O₂), OC(O)O, OC(O)S, OC(O)N(Rₐ), N(Rₐ)C(O)O, N(Rₐ)C(O)S, N(Rₐ)C(O)N(Rₐ), (CRₐR_{b})ₚN(Rₐ)(CRₐR_{b})_{q}, (CRₐR_{b})ₚN(Rₐ)C(O)(CRₐR_{b})_{q}, OC(O)N(R_{b})(CRₐR_{b})ₚ₊₁N(R_{b})(CRₐR_{b})_{q}, or (CRₐR_{b})ₚC(O)N(Rₐ)(CRₐR_{b})_{q};
Z₅ is a bond, (CRₐR_{b})ₚ, a bivalent alkenyl group, or a bivalent alkynyl group, N(Rₐ), O, S, C(O), S(O₂), —O(CRₐR_{b})ₚ—, —N(Rₐ)(CRₐR_{b})ₚ—, OC(O), C(O)O, OSO₂, S(O₂)O, C(O)S, SC(O), C(O)C(O), C(O)N(Rₐ), N(Rₐ)C(O), S(O₂)N(Rₐ), N(Rₐ)S(O₂), OC(O)O, OC(O)S, OC(O)N(Rₐ), N(Rₐ)C(O)O, N(Rₐ)C(O)S, N(Rₐ)C(O)N(Rₐ), (CRₐR_{b})ₚN(Rₐ)(CRₐR_{b})_{q}, (CRₐR_{b})ₚN(Rₐ)C(O)(CRₐR_{b})_{q}, OC(O)N(R_{b})(CRₐR_{b})ₚ₊₁N(R_{b})(CRₐR_{b})_{q}, or (CRₐR_{b})ₚC(O)N(Rₐ)(CRₐR_{b})_{q};
L is absent, a bond, (CRₐR_{b})ₚ, N(R_{c}), O, S, C(O), S(O₂), —O(CRₐR_{b})ₚ—, —N(R_{c})(CRₐR_{b})ₚ—, OC(O), C(O)O, OSO₂, S(O₂)O, C(O)S, SC(O), C(O)C(O), C(O)N(R_{c}), N(R_{c})C(O), S(O₂)N(R_{c}), N(R_{c})S(O₂), OC(O)O, OC(O)S, OC(O)N(R_{c}), N(R_{c})C(O)O, N(R_{c})C(O)S, N(R_{c})C(O)N(R_{c}), (CRₐR_{b})ₚN(R_{c})(CRₐR_{b})_{q}, (CRₐR_{b})ₚN(R_{c})C(O)(CRₐR_{b})_{q}, OC(O)N(R_{c})(CRₐR_{b})ₚ₊₁N(R_{c})(CRₐR_{b})_{q}, (CRₐR_{b})ₚC(O)N(R_{c})(CRₐR_{b})_{q}, bivalent alkenyl, bivalent alkynyl, bivalent cycloalkyl, bivalent cycloalkenyl, bivalent spirocycloalkyl, bivalent fused-carbocyclic, bivalent bridged-carbocyclic, bivalent heterocycloalkyl, bivalent heterocycloalkenyl, bivalent spiro-heterocyclic, bivalent fused-heterocyclic, bivalent bridged-heterocyclic, bivalent aryl, or bivalent heteroaryl, each of the aforementioned is independently optionally substituted with one or more R_{d};
each Rₐ is independently H, D, halo, cyano, nitro, alkyl, alkylene-P(O)RₑR_{f}, alkenyl, alkynyl, —C(O)alkyl, —C(O)NHOH, —C(O)NH₂, —C(O)OH, —C(O)O(alkyl), —NH₂, —NH(alkyl), —NH(haloalkyl), —NRₑR_{f}, —NHC(O)alkyl, =NRₑ, —N═S(O)RₑR_{f}, —OH, —O(alkyl), ═O, —P(O)RₑR_{f}, —S(O)(NRₑ)R_{f}, cycloalkyl, cycloalkenyl, spirocycloalkyl, fused-carbocyclic, bridged-carbocyclic, heterocycloalkyl, heterocycloalkenyl, spiro-heterocyclic, fused-heterocyclic, bridged-heterocyclic, aryl, or heteroaryl, each of the aforementioned is optionally and independently substituted with one or more independently selected Rₑ substituents;
each R_{b} is independently H, D, halo, cyano, nitro, alkyl, alkylene-P(O)RₑR_{f}, alkenyl, alkynyl, —C(O)alkyl, —C(O)NHOH, —C(O)NH₂, —C(O)OH, —C(O)O(alkyl), —NH₂, —NH(alkyl), —NH(haloalkyl), —NRₑR_{f}, —NHC(O)alkyl, =NRₑ, —N═S(O)RₑR_{f}, —OH, —O(alkyl), ═O, —P(O)RₑR_{f}, —S(O)(NRₑ)R_{f}, cycloalkyl, cycloalkenyl, spirocycloalkyl, fused-carbocyclic, bridged-carbocyclic, heterocycloalkyl, heterocycloalkenyl, spiro-heterocyclic, fused-heterocyclic, bridged-heterocyclic, aryl, or heteroaryl, each of the aforementioned is optionally and independently substituted with one or more independently selected Rₑ substituents;
each R_{c} is independently H, D, halo, cyano, nitro, alkyl, alkylene-P(O)RₑR_{f}, alkenyl, alkynyl, —C(O)alkyl, —C(O)NHOH, —C(O)NH₂, —C(O)OH, —C(O)O(alkyl), —NH₂, —NH(alkyl), —NH(haloalkyl), —NRₑR_{f}, —NHC(O)alkyl, =NRₑ, —N═S(O)RₑR_{f}, —OH, —O(alkyl), ═O, —P(O)RₑR_{f}, —S(O)(NRₑ)R_{f}, cycloalkyl, cycloalkenyl, spirocycloalkyl, fused-carbocyclic, bridged-carbocyclic, heterocycloalkyl, heterocycloalkenyl, spiro-heterocyclic, fused-heterocyclic, bridged-heterocyclic, aryl, or heteroaryl, each of the aforementioned is optionally and independently substituted with one or more independently selected Rₑ substituents;
R_{b} and R_{c}, taken together with the atom(s) to which they are attached, independently form a cycloalkyl or heterocycloalkyl, wherein each cycloalkyl or heterocycloalkyl is optionally and independently substituted with one or more independently selected Rₑ substituents;
each R_{d} is independently H, D, halo, cyano, nitro, alkyl, alkylene-P(O)RₑR_{f}, alkenyl, alkynyl, —C(O)alkyl, —C(O)NHOH, —C(O)NH₂, —C(O)OH, —C(O)O(alkyl), —NH₂, —NH(alkyl), —NH(haloalkyl), —NRₑR_{f}, —NHC(O)alkyl, =NRₑ, —N═S(O)RₑR_{f}, —OH, —O(alkyl), ═O, —P(O)RₑR_{f}, —S(O)(NRₑ)R_{f}, cycloalkyl, cycloalkenyl, spirocycloalkyl, fused-carbocyclic, bridged-carbocyclic, heterocycloalkyl, heterocycloalkenyl, spiro-heterocyclic, fused-heterocyclic, bridged-heterocyclic, aryl, or heteroaryl, each of the aforementioned is optionally and independently substituted with one or more independently selected Rₑ substituents; or
two R_{d}, taken together with the atom(s) to which they are attached, independently form a cycloalkyl or heterocycloalkyl, wherein each cycloalkyl or heterocycloalkyl is optionally and independently substituted with one or more independently selected Rₑ substituents;
each Rₑ is independently H, D, halo, cyano, nitro, alkyl, alkylene, alkenyl, alkynyl, —C(O)alkyl, —C(O)NHOH, —C(O)NH₂, —C(O)OH, —C(O)O(alkyl), —NH₂, —NH(alkyl), —N(alkyl)(alkyl), —NH(haloalkyl), —NHC(O)alkyl, =N(alkyl), —N═S(O) (alkyl)(alkyl), —OH, —O(alkyl), =O, cycloalkyl, cycloalkenyl, spirocycloalkyl, fused-carbocyclic, bridged-carbocyclic, heterocycloalkyl, heterocycloalkenyl, spiro-heterocyclic, fused-heterocyclic, bridged-heterocyclic, aryl, or heteroaryl, each of the aforementioned is optionally and independently substituted with one or more independently selected R_{f} substituents; or
two Rₑ, taken together with the atom(s) to which they are attached, independently form a cycloalkyl or heterocycloalkyl, wherein each cycloalkyl or heterocycloalkyl is optionally and independently substituted with one or more independently selected R_{f} substituents;
each R_{f} is independently H, D, halo, cyano, nitro, alkyl, alkylene, alkenyl, alkynyl, —C(O)alkyl, —C(O)NHOH, —C(O)NH₂, —C(O)OH, —C(O)O(alkyl), —NH₂, —NH(alkyl), —N(alkyl)(alkyl), —NH(haloalkyl), —NHC(O)alkyl, =N(alkyl), —N═S(O) (alkyl)(alkyl), —OH, —O(alkyl), =O, cycloalkyl, cycloalkenyl, spirocycloalkyl, fused-carbocyclic, bridged-carbocyclic, heterocycloalkyl, heterocycloalkenyl, spiro-heterocyclic, fused-heterocyclic, bridged-heterocyclic, aryl, or heteroaryl, each of the aforementioned is optionally and independently substituted with one or more independently selected R_{g} substituents;
Rₑ and R_{f}, taken together with the atom(s) to which they are attached, independently form a cycloalkyl or heterocycloalkyl, wherein each cycloalkyl or heterocycloalkyl is optionally and independently substituted with one or more independently selected R_{g} substituents;
each R_{g} is independently H, D, halo, cyano, nitro, alkyl, alkylene, alkenyl, alkynyl, —C(O)alkyl, —C(O)NHOH, —C(O)NH₂, —C(O)OH, —C(O)O(alkyl), —NH₂, —NH(alkyl), —N(alkyl)(alkyl), —NH(haloalkyl), NHC(O)alkyl, =N(alkyl), —N═S(O) (alkyl)(alkyl), —OH, —O(alkyl), =O, cycloalkyl, cycloalkenyl, spirocycloalkyl, fused-carbocyclic, bridged-carbocyclic, heterocycloalkyl, heterocycloalkenyl, spiro-heterocyclic, fused-heterocyclic, bridged-heterocyclic, aryl, or heteroaryl;
a is 0, 1,2,3,4,5,6,7, or 8;
b is 0, 1,2,3,4,5,6,7, or 8;
c is 0, 1,2,3,4,5,6, 7, or 8;
g is 0, 1,2,3,4,5,6,7, or 8;
j is 0, 1, 2, 3, 4, 5, 6, 7, or 8;
k is 0, 1,2,3,4,5,6, 7, or 8;
m is 0, 1, 2, 3, 4, 5, 6, 7, or 8;
n is 0, 1, 2, 3, 4, 5, 6, 7, or 8;
v is 0, 1,2,3,4,5,6, 7, or 8;
u is 0, 1, 2, 3, 4, or 5;
f is 0, 1, or 2;
p is 0, 1, 2, 3, 4, or 5; and
q is 0, 1, 2, 3, 4, or 5.

2. The compound according to claim 1 or an N-oxide thereof, or a pharmaceutically acceptable salt, solvate, polymorph, tautomer, stereoisomer, an isotopic form, or a prodrug thereof, wherein the compound is represented by Formula (2): 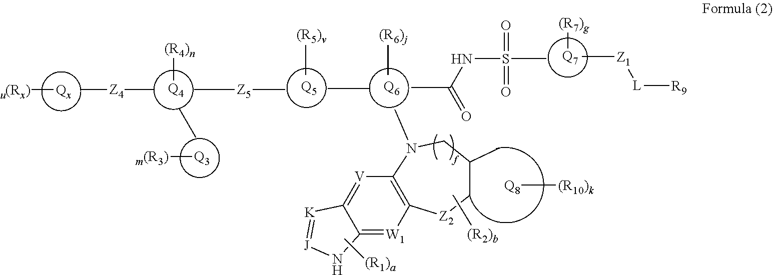
wherein
each of W₁, V, K, J, independently, is C(Rₐ) or N.

3. The compound according to claim 2 or an N-oxide thereof, or a pharmaceutically acceptable salt, solvate, polymorph, tautomer, stereoisomer, an isotopic form, or a prodrug thereof, wherein the compound is represented by Formula (3): 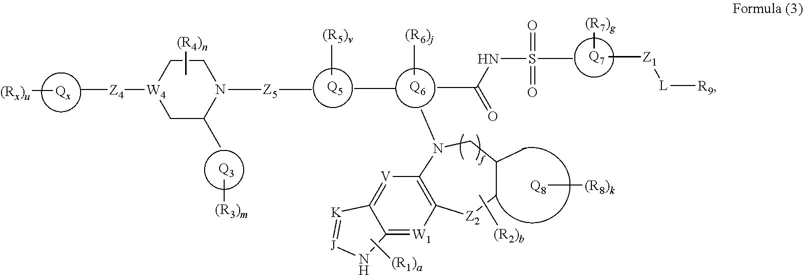
wherein
W₄ is C(Rₐ) or N.

4. The compound according to claim 3 or an N-oxide thereof, or a pharmaceutically acceptable salt, solvate, polymorph, tautomer, stereoisomer, an isotopic form, or a prodrug thereof, wherein the compound is represented by Formula (4): 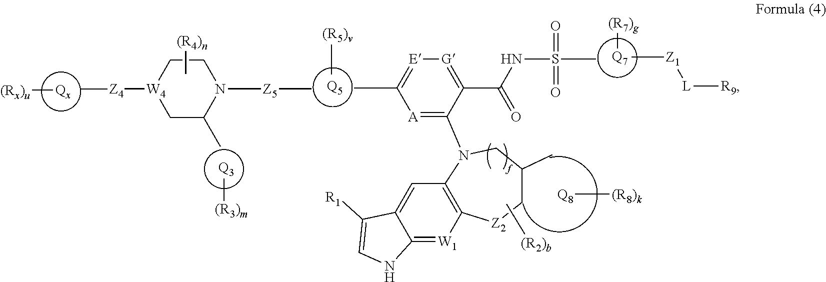
wherein
each of A, E′, and G′, independently is C(Rₐ) or N.

5. The compound according to claim 4 or an N-oxide thereof, or a pharmaceutically acceptable salt, solvate, polymorph, tautomer, stereoisomer, an isotopic form, or a prodrug thereof, wherein the compound is represented by Formula (5): 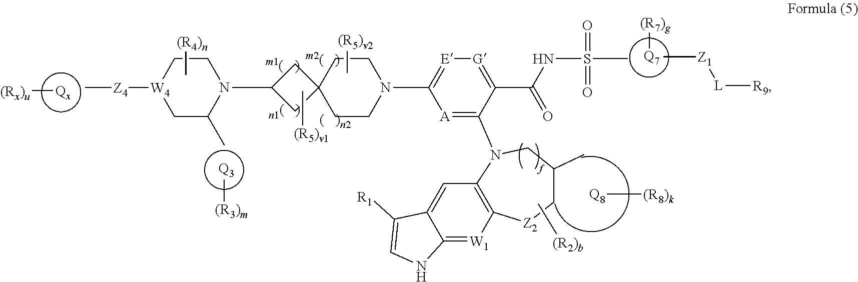
wherein
each of m1, m2, n1, and n2, independently, is 0, 1, or 2; and
v1 and v2 are both integers including 0, and v1+v2=v.

6. The compound according to claim 5 or an N-oxide thereof, or a pharmaceutically acceptable salt, solvate, polymorph, tautomer, stereoisomer, an isotopic form, or a prodrug thereof, wherein the compound is represented by Formula (6):

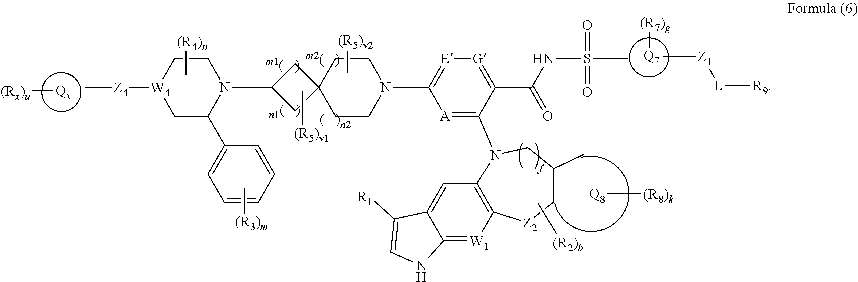

7. The compound according to claim 6 or an N-oxide thereof, or a pharmaceutically acceptable salt, solvate, polymorph, tautomer, stereoisomer, an isotopic form, or a prodrug thereof, wherein the compound is represented by Formula (7): 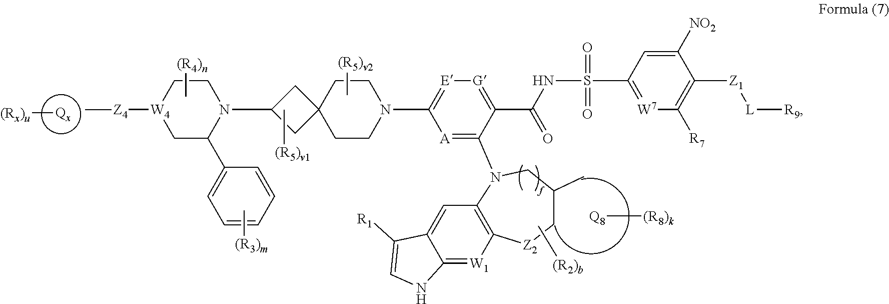
wherein
W⁷ is N, or C(Rₐ).

8. The compound according to claim 6 or an N-oxide thereof, or a pharmaceutically acceptable salt, solvate, polymorph, tautomer, stereoisomer, an isotopic form, or a prodrug thereof, wherein the compound is represented by Formula (8): 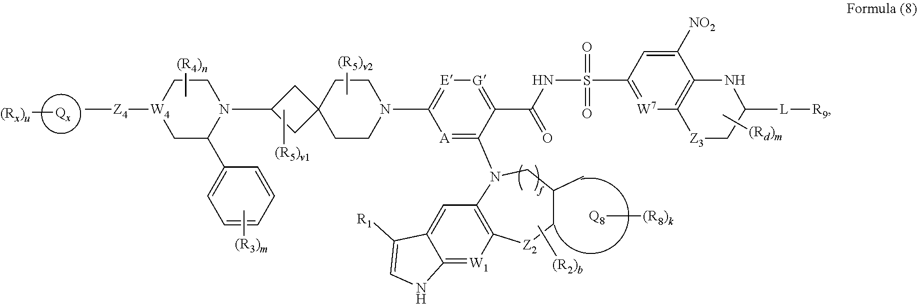
wherein
W⁷ is N, or C(Rₐ); and
Z₃ is a bond, (CH₂)ₚ, N(H), O, S, C(O), S(O₂), OC(O), C(O)O, OSO₂, S(O₂)O, C(O)S, SC(O), C(O)C(O), C(O)N(H), N(H)C(O), S(O₂)N(H), N(H)S(O₂), OC(O)O, OC(O)S, OC(O)N(H), N(H)C(O)O, N(H)C(O)S, N(H)C(O)N(H), (CH₂)ₚN(H)(CH₂)_{q}, (CH₂)ₚN(H)C(O)(CH₂)_{q}, (CH₂)ₚC(O)N(H)(CH₂)_{q}, or OC(O)N(H)(CH₂)ₚ₊₁N(H)(CH₂)_{q}.

9. A pharmaceutical composition comprising a compound of any one of Formulae (1), or an N-oxide thereof as defined in any one of claims 1-8, or a pharmaceutically acceptable salt, solvate, polymorph, tautomer, stereoisomer, an isotopic form, or a prodrug of said compound of any one of Formulae (1)-(8) or N-oxide thereof, and a pharmaceutically acceptable diluent or carrier.

10. A method of treating a neoplastic disease, an autoimmune disease, or a neurodegenerative disease, comprising administering to a subject in need thereof an effective amount of a compound of any one of Formulas (1)-(8), or an N-oxide thereof as defined in any one of claims 1-9, or a pharmaceutically acceptable salt, solvate, polymorph, tautomer, stereoisomer, an isotopic form, or a prodrug of said compound of any one of Formulae (1)-(8) or N-oxide thereof.
